(19) United States
(12) Patent Application Publication
(10) Pub. No.: US 2003/0208039 A1
(43) Pub. Date: Nov. 6, 2003

(54) Novel antibodies that bind to antigenic polypeptides, nucleic acids encoding the antigens, and methods of use

(76) Inventors: Muralidhara Padigaru, Branford, CT (US); Suresh G. Shenoy, Branford, CT (US); Remesh Kekuda, Norwalk, CT (US); Vladimir Gusev, Madison, CT (US); Pascale F-J Pochart, Madison, CT (US); Mei Zhong, Branford, CT (US); Luca Rastelli, Guilford, CT (US); Peter D. Mezes, Old Lyme, CT (US); Glennda Smithson, Guilford, CT (US); Xiaojia Sasha Guo, Branford, CT (US); Valerie Gerlach, Branford, CT (US); Stacie J. Casman, North Haven, CT (US); Ferenc L. Boldog, North Haven, CT (US); Li Li, Branford, CT (US); Bryan D. Zerhusen, Branford, CT (US); Velizar T. Tchernev, Branford, CT (US); Esha A. Gangolli, Madison, CT (US); Corine A.M. Vernet, Branford, CT (US); Carol E. A. Pena, New Haven, CT (US); Catherine E. Burgess, Wethersfield, CT (US); Xiaohong Liu, Branford, CT (US); Kimberly A. Spytek, New Haven, CT (US); Linda Gorman, Branford, CT (US); Steven K. Spaderna, Berlin, CT (US); Edward Z. Voss, Wallingford, CT (US); Uriel M. Malyankar, Branford, CT (US); David W. Anderson, Branford, CT (US); Meera Patturajan, Branford, CT (US); Charles E. Miller, Guilford, CT (US); Raymond J. Taupier JR., East Haven, CT (US)

Correspondence Address: Ivor R. Elrifi, Esq., MINTZ, LEVIN, COHN, FERRIS,, GLOVSKY and POPEO, P.C., One Financial Center, Boston, MA, 02111, US

(21) Appl. No.: 10/093,463

(22) Filed: Mar. 8, 2002

Related U.S. Application Data
(60) Provisional application No. 60/274,322, filed on Mar. 8, 2001.
(60) Provisional application No. 60/283,675, filed on Apr. 13, 2001.
(60) Provisional application No. 60/338,092, filed on Dec. 3, 2001.
(60) Provisional application No. 60/274,281, filed on Mar. 8, 2001.
(60) Provisional application No. 60/274,101, filed on Mar. 7, 2001.
(60) Provisional application No. 60/325,681, filed on Sep. 27, 2001.
(60) Provisional application No. 60/304,354, filed on Jul. 10, 2001.
(60) Provisional application No. 60/279,995, filed on Mar. 30, 2001.
(60) Provisional application No. 60/294,899, filed on May 31, 2001.
(60) Provisional application No. 60/287,424, filed on Apr. 30, 2001.
(60) Provisional application No. 60/299,027, filed on Jun. 18, 2001.
(60) Provisional application No. 60/309,198, filed on Jul. 31, 2001.
(60) Provisional application No. 60/281,194, filed on Apr. 3, 2001.
(60) Provisional application No. 60/274,194, filed on Mar. 8, 2001.
(60) Provisional application No. 60/274,849, filed on Mar. 9, 2001.
(60) Provisional application No. 60/330,380, filed on Oct. 18, 2001.
(60) Provisional application No. 60/275,235, filed on Mar. 12, 2001.
(60) Provisional application No. 60/288,342, filed on May 3, 2001.
(60) Provisional application No. 60/275,578, filed on Mar. 13, 2001.
(60) Provisional application No. 60/291,240, filed on May 16, 2001.
(60) Provisional application No. 60/294,485, filed on May 30, 2001.
(60) Provisional application No. 60/299,310, filed on Jun. 19, 2001.
(60) Provisional application No. 60/275,579, filed on Mar. 13, 2001.
(60) Provisional application No. 60/275,601, filed on Mar. 13, 2001.
(60) Provisional application No. 60/276,000, filed on Mar. 14, 2001.
(60) Provisional application No. 60/280,900, filed on Apr. 2, 2001.
(60) Provisional application No. 60/276,776, filed on Mar. 16, 2001.
(60) Provisional application No. 60/294,889, filed on May 31, 2001.
(60) Provisional application No. 60/318,770, filed on Sep. 12, 2001.
(60) Provisional application No. 60/276,994, filed on Mar. 19, 2001.
(60) Provisional application No. 60/277,338, filed on Mar. 20, 2001.
(60) Provisional application No. 60/325,430, filed on Sep. 27, 2001.
(60) Provisional application No. 60/332,094, filed on Nov. 21, 2001.
(60) Provisional application No. 60/299,303, filed on Jun. 19, 2001.
(60) Provisional application No. 60/288,066, filed on May 2, 2001.
(60) Provisional application No. 60/277,321, filed on Mar. 20, 2001.
(60) Provisional application No. 60/280,822, filed on Apr. 2, 2001.
(60) Provisional application No. 60/277,239, filed on Mar. 20, 2001.
(60) Provisional application No. 60/277,327, filed on Mar. 20, 2001.
(60) Provisional application No. 60/277,791, filed on Mar. 21, 2001.
(60) Provisional application No. 60/333,184, filed on Nov. 14, 2001.
(60) Provisional application No. 60/277,833, filed on Mar. 22, 2001.
(60) Provisional application No. 60/318,462, filed on Sep. 10, 2001.
(60) Provisional application No. 60/288,528, filed on May 3, 2001.
(60) Provisional application No. 60/278,152, filed on Mar. 23, 2001.
(60) Provisional application No. 60/332,272, filed on Nov. 14, 2001.
(60) Provisional application No. 60/278,894, filed on Mar. 26, 2001.
(60) Provisional application No. 60/312,903, filed on Aug. 16, 2001.
(60) Provisional application No. 60/333,272, filed on Nov. 14, 2001.
(60) Provisional application No. 60/279,036, filed on Mar. 27, 2001.
(60) Provisional application No. 60/332,172, filed on Nov. 14, 2001.
(60) Provisional application No. 60/337,426, filed on Dec. 3, 2001.
(60) Provisional application No. 60/278,999, filed on Mar. 27, 2001.
(60) Provisional application No. 60/279,344, filed on Mar. 28, 2001.
(60) Provisional application No. 60/332,271, filed on Nov. 14, 2001.
(60) Provisional application No. 60/291,099, filed on May 16, 2001.
(60) Provisional application No. 60/291,190, filed on May 15, 2001.
(60) Provisional application No. 60/280,233, filed on Mar. 30, 2001.
(60) Provisional application No. 60/280,802, filed on Apr. 2, 2001.
(60) Provisional application No. 60/335,301, filed on Oct. 31, 2001.
(60) Provisional application No. 60/337,185, filed on Dec. 4, 2001.
(60) Provisional application No. 60/345,705, filed on Jan. 3, 2002.

(51) Int. Cl. C07K 14/435; C07H 21/04; C12P 21/02; C12N 5/06

(52) U.S. Cl. 530/350; 435/69.1; 536/23.5; 435/320.1; 435/325

(57) ABSTRACT

Disclosed herein are nucleic acid sequences that encode polypeptides. Also disclosed are antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids, polypeptides, or antibodies, or fragments thereof.

### RELATED APPLICATIONS

This application claims priority to U.S. Ser. No. 60/274,322 filed on Mar. 8, 2001 (Cura 590); U.S. Ser. No. 60/283,675 filed on Apr. 13, 2001 (Cura 590D1); U.S. Ser. No. 60/338,092 filed on Dec. 3, 2001 (Cura 590D2); U.S. Ser. No. 60/274,281 filed on Mar. 8, 2001 (Cura 591); U.S. Ser. No. 60/274,101 filed on Mar. 8, 2001 (Cura 592); U.S. Ser. No. 60/325,681 filed on Sep. 27, 2001 (Cura 592J1); U.S. Ser. No. 60/304,354 filed on Jul. 10, 2001 (Cura 592I1); U.S. Ser. No. 60/279,995 filed on Mar. 30, 2001 (Cura 592H1); U.S. Ser. No. 60/294,899 filed on May 31, 2001 (Cura 592E1); U.S. Ser. No. 60/287,424 filed on Apr. 30,2001 (Cura 592D1); U.S. Ser. No. 60/299,027 filed on Jun. 18, 2001 (Cura 592D2); U.S. Ser. No. 60/309,198 filed on Jul. 31, 2001 (Cura 592C1); U.S. Ser. No. 60/281,194 filed on Apr. 4, 2001 (Cura 592A1); U.S. Ser. No. 60/274,194 filed on Mar. 8, 2001 (Cura 593); U.S. Ser. No. 60/274,849 filed on Mar. 9, 2001 (Cura 594); U.S. Ser. No. 60/330,380 filed on Oct. 18, 2001 (Cura 594C1); U.S. Ser. No. 60/275,235 filed on Mar. 12, 2001 (Cura 595); U.S. Ser. No. 60/288,342 filed on May 3, 2001 (Cura 595J1); U.S. Ser. No. 60/275,578 filed on Mar. 13, 2001 (Cura 596); U.S. Ser. No. 60/291,240 filed on May 16, 2001 (Cura 596I1); U.S. Ser. No. 60/294,485 filed on May 30,2001 (Cura 596B1); U.S. Ser. No. 60/299,310 filed on Jun. 19, 2001 (Cura 596A1); U.S. Ser. No. 60/275,579 filed on Mar. 13, 2001 (Cura 597); U.S. Ser. No. 60/275,601 filed on Mar. 13, 2001 (Cura 598); U.S. Ser. No. 60/276,000 filed on Mar. 14, 2001 (Cura 599); U.S. Ser. No. 60/280,900 filed on Apr. 2, 2001 (Cura 599E1); U.S. Ser. No. 60/276,776 filed on Mar. 16, 2001 (Cura 600); U.S. Ser. No. 60/294,889 filed on May 31, 2001 (Cura 600G1); U.S. Ser. No. 60/318,770 filed on Sep. 12, 2001 (Cura 600E1); U.S. Ser. No. 60/276,994 filed on Mar. 19, 2001 (Cura 604); U.S. Ser. No. 60/277,338 filed on Mar. 20, 2001 (Cura 607); U.S. Ser. No. 60/325,430 filed on Sep. 27, 2001 (Cura 607J1); U.S. Ser. No. 60/332,094 filed on Nov. 21, 2001 (Cura 607C1); U.S. Ser. No. 60/299,303 filed on Jun. 19, 2001 (Cura 607B1); U.S. Ser. No. 60/288,066 filed on May 2, 2001 (Cura 607A1); U.S. Ser. No. 60/277,321 filed on Mar. 20, 2001 (Cura 608); U.S. Ser. No. 60/280,822 filed on Apr. 2, 2001 (Cura 608A); U.S. Ser. No. 60/277,239 filed on Mar. 20, 2001 (Cura 609); U.S. Ser. No. 60/277,327 filed on Mar. 20, 2001 (Cura 610); U.S. Ser. No. 60/277,791 filed on Mar. 21, 2001 (Cura 611); U.S. Ser. No. 60/333,184 filed on Nov. 14, 2001 (Cura 611H1); U.S. Ser. No. 60/277,833 filed on Mar. 22, 2001 (Cura 612); U.S. Ser. No. 60/318,462 filed on Sep. 10, 2001 (Cura 612J1); U.S. Ser. No. 60/288,528 filed on May 3, 2001 (Cura 612A1); U.S. Ser. No. 60/278,152 filed on Mar. 23, 2001 (Cura 613); U.S. Ser. No. 60/332,272 filed on Nov. 14, 2001 (Cura 613D1); U.S. Ser. No. 60/278,894 filed on Mar. 26, 2001 (Cura 614); U.S. Ser. No. 60/312,903 filed on Aug. 16, 2001 (Cura 614C1); U.S. Ser. No. 60/333,272 filed on Nov. 14, 2001 (Cura 614C2); U.S. Ser. No. 60/279,036 filed on Mar. 27, 2001 (Cura 615); U.S. Ser. No. 60/332,172 filed on Nov. 14, 2001 (Cura 615I1); U.S. Ser. No. 60/337,426 filed on Dec. 3, 2001 (Cura 615I2); U.S. Ser. No. 60/278,999 filed on Mar. 27, 2001 (Cura 616); U.S. Ser. No. 60/279,344 filed on Mar. 28, 2001 (Cura 617); U.S. Ser. No. 60/332,271 filed on Nov. 14, 2001 (Cura 617J1); U.S. Ser. No. 60/291,099 filed on May 16, 2001 (Cura 617H1); U.S. Ser. No. 60/291,190 filed on May 15, 2001 (Cura 617E1); U.S. Ser. No. 60/280,233 filed on Mar. 30, 2001 (Cura 618); U.S. Ser. No. 60/280,802 filed on Apr. 2, 2001 (Cura 621); U.S. Ser. No. 60/335,301 filed on Oct. 31, 2001 (Cura 621F1); U.S. Ser. No. 60/337,185 filed on Dec. 4, 2001 (Cura 621D1); and U.S. Ser. No. 60/345,705 filed on Jan. 3, 2002 (Cura 621B1), each of which is incorporated by reference in its entirety.

### FIELD OF THE INVENTION

The present invention relates to novel antibodies that bind immunospecifically to antigenic polypeptides, wherein the polypeptides have characteristic properties related to biochemical or physiological responses in a cell, a tissue, an organ or an organism. The novel polypeptides are gene products of novel genes, or are specified biologically active fragments or derivatives thereof. Methods of use of the antibodies encompass procedures for diagnostic and prognostic assay of the polypeptides, as well as methods of treating diverse pathological conditions.

### BACKGROUND OF THE INVENTION

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways are constituted of extracellular signaling proteins, cellular receptors that bind the signaling proteins and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including by way of nonlimiting example induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as elevated or excessive synthesis and secretion of protein effectors. In a clinical setting a subject may be suspected of suffering from a condition brought on by elevated or excessive levels of a protein effector of interest.

Antibodies are multichain proteins that bind specifically to a given antigen, and poorly or not at all to substances deemed not to be a cognate antigen. Antibodies are comprised of two short chains termed light chains and two long chains termed heavy chains. These chains are constituted of immunoglobulin domains, of which generally there are two classes: one variable domain per chain and one constant domain in light chains and three or more constant domains in heavy chains. The antigen-specific portion of the immunoglobulin molecules resides in the variable domains; the variable domains of one light chain and one heavy chain associate with each other to generate the antigen-binding moiety. Antibodies that bind immunospecifically to a cognate or target antigen bind with high affinities. Accordingly, they are useful in assaying specifically for the presence of the antigen in a sample. In addition, they have the potential of inactivating the activity of the antigen.

Therefore there is a need to assay for the level of the protein effector of interest in a biological sample from such a subject, and to compare the level with that characteristic of a nonpathological condition. In particular, there is a need for such an assay based on the use of an antibody that binds immunospecifically to the antigen. There further is a need to inhibit the activity of the protein effector in cases where a pathological condition arises from elevated or excessive levels of the effector based on the use of an antibody that binds immunospecifically to the effector. Thus there is a need for the antibody as a product of manufacture. There further is a need for a method of treatment of a pathological condition brought on by an elevated or excessive level of the protein effector of interest based on administering the antibody to the subject.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, etc. nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated polypeptide comprising a mature form of a NOVX amino acid. The polypeptide can be, for example, a NOVX amino acid sequence or a variant of a NOVX amino acid sequence, wherein any amino acid specified in the chosen sequence is changed to a different amino acid, provided that no more than 15% of the amino acid residues in the sequence are so changed. The invention also includes fragments of any of NOVX polypeptides. In another aspect, the invention also includes an isolated nucleic acid that encodes a NOVX polypeptide, or a fragment, homolog, analog or derivative thereof.

Also included in the invention is a NOVX polypeptide that is a naturally occurring variant of a NOVX sequence. In one embodiment, the variant includes an amino acid sequence that is the translation of a nucleic acid sequence differing by a single nucleotide from a NOVX nucleic acid sequence. In another embodiment, the NOVX polypeptide is a variant polypeptide described therein, wherein any amino acid specified in the chosen sequence is changed to provide a conservative substitution.

In another aspect, invention provides a method for determining the presence or amount of the NOVX polypeptide in a sample by providing a sample; introducing the sample to an antibody that binds immunospecifically to the polypeptide; and determining the presence or amount of antibody bound to the NOVX polypeptide, thereby determining the presence or amount of the NOVX polypeptide in the sample.

In yet another aspect, the invention includes a method for determining the presence of or predisposition to a disease associated with altered levels of a NOVX polypeptide in a mammalian subject by measuring the level of expression of the polypeptide in a sample from the first mammalian subject; and comparing the amount of the polypeptide in the sample of the first step to the amount of the polypeptide present in a control sample from a second mammalian subject known not to have, or not to be predisposed to, the disease. An alteration in the expression level of the polypeptide in the first subject as compared to the control sample indicates the presence of or predisposition to the disease.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In still another aspect, the invention provides the use of a therapeutic in the manufacture of a medicament for treating a syndrome associated with a human disease that is associated with a NOVX polypeptide.

In a further aspect, the invention provides a method for modulating the activity of a NOVX polypeptide by contacting a cell sample expressing the NOVX polypeptide with antibody that binds the NOVX polypeptide in an amount sufficient to modulate the activity of the polypeptide.

The invention also includes an isolated nucleic acid that encodes a NOVX polypeptide, or a fragment, homolog, analog or derivative thereof. In a preferred embodiment, the nucleic acid molecule comprises the nucleotide sequence of a naturally occurring allelic nucleic acid variant. In another embodiment, the nucleic acid encodes a variant polypeptide, wherein the variant polypeptide has the polypeptide sequence of a naturally occurring polypeptide variant. In another embodiment, the nucleic acid molecule differs by a single nucleotide from a NOVX nucleic acid sequence. In one embodiment, the NOVX nucleic acid molecule hybridizes under stringent conditions to the nucleotide sequence selected from the group consisting of SEQ ID NO: 2n−1, wherein n is an integer between 1 and 101, or a complement of the nucleotide sequence. In one embodiment, the invention provides a nucleic acid molecule wherein the nucleic acid includes the nucleotide sequence of a naturally occurring allelic nucleic acid variant.

Also included in the invention is a vector containing one or more of the nucleic acids described herein, and a cell containing the vectors or nucleic acids described herein. The invention is also directed to host cells transformed with a vector comprising any of the nucleic acid molecules described above.

In yet another aspect, the invention provides for a method for determining the presence or amount of a nucleic acid molecule in a sample by contacting a sample with a probe that binds a NOVX nucleic acid and determining the amount of the probe that is bound to the NOVX nucleic acid. For example the NOVX nucleic may be a marker for cell or tissue type such as a cell or tissue type that is cancerous.

In yet a further aspect, the invention provides a method for determining the presence of or predisposition to a disease associated with altered levels of a nucleic acid molecule in a first mammalian subject, wherein an alteration in the level of the nucleic acid in the first subject as compared to the control sample indicates the presence of or predisposition to the disease.

The invention further provides an antibody that binds immunospecifically to a NOVX polypeptide. The NOVX antibody may be monoclonal, humanized, or a fully human antibody. Preferably, the antibody has a dissociation constant for the binding of the NOVX polypeptide to the antibody less than 1×10⁻⁹ M. More preferably, the NOVX antibody neutralizes the activity of the NOVX polypeptide.

In a further aspect, the invention provides for the use of a therapeutic in the manufacture of a medicament for treating a syndrome associated with a human disease, associated with a NOVX polypeptide. Preferably the therapeutic is a NOVX antibody.

In yet a further aspect, the invention provides a method of treating or preventing a NOVX-associated disorder, a method of treating a pathological state in a mammal, and a method of treating or preventing a pathology associated with a polypeptide by administering a NOVX antibody to a subject in an amount sufficient to treat or prevent the disorder.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table 1 provides a summary of the NOVX nucleic acids and their encoded polypeptides.

TABLE 1
NOVX Polynucleotide and Polypeptide Sequences and Corresponding
SEQ ID Numbers

| | | SEQ ID | SEQ ID | |
|---|---|---|---|---|
| NOVX | | NO | NO | |
| Assign- | Internal | (nucleic | (poly- | |
| ment | Identification | acid) | peptide) | Homology |
| 1a | CG58546-01 | 1 | 2 | Adlican |
| 1b | 174307918 | 3 | 4 | Adlican |
| 1c | 174307924 | 5 | 6 | Adlican |
| 1d | 169679197 | 7 | 8 | Adlican |
| 1e | 169679219 | 9 | 10 | Adlican |
| 1f | 207704655 | 11 | 12 | Adlican |
| 2a | CG58598-01 | 13 | 14 | Brain Specific Trans- |
| | | | | membrane-like |
| 2b | CG58598-02 | 15 | 16 | Brain Specific Trans- |
| | | | | membrane-like |
| 2c | 209770459 | 17 | 18 | Brain Specific Trans- |
| | | | | membrane-like |
| 3a | CG57833-01 | 19 | 20 | Amino Acid Transporter-like |
| 4a | CG57853-01 | 21 | 22 | Heal Na(+)/Bile Cotrans- |
| | | | | porter-like |
| 4b | CG57853-02 | 23 | 24 | Heal Na(+)/Bile Cotrans- |
| | | | | porter-like |
| 4c | CG57853-03 | 25 | 26 | Heal Na(+)/Bile Cotrans- |
| | | | | porter-like |
| 5a | CG57829-01 | 27 | 28 | ADAM-TS 1 Precursor-like |
| 5b | CG57829-05 | 29 | 30 | ADAM-TS 1 Precursor-like |
| 5c | 175070495 | 31 | 32 | ADAM-TS 1 Precursor-like |
| 5d | 175070504 | 33 | 34 | ADAM-TS 1 Prccursor-like |
| 5e | 175070512 | 35 | 36 | ADAM-TS 1 Precursor-like |
| 5f | 175070519 | 37 | 38 | ADAM-TS 1 Precursor-like |
| 6a | CG59197-01 | 39 | 40 | TULIP 2-like (Tuberin) |
| 6b | 188822075 | 41 | 42 | TULIP 2-like (Tuberin) |
| 7a | CG58524-01 | 43 | 44 | T cell receptor beta chain |
| | | | | precursor V region-like |
| 8a | CG56512-01 | 45 | 46 | Oncofetal Antigen |
| | | | | Precursor -like |
| 9a | CG58180-01 | 47 | 48 | Prohibitin-like |
| 10a | CG59199-01 | 49 | 50 | Natriuretic Peptide Receptor |
| 11a | CG59249-01 | 51 | 52 | Metalloproteinase Disintegrin |
| | | | | beta (ADAM) |
| 11b | CG59249-02 | 53 | 54 | Metalloproteinase Disintegrin |
| | | | | beta (ADAM) |
| 12a | CG58577-01 | 55 | 56 | CASPR4 |
| 12b | 174307971 | 57 | 58 | CASPR4 |
| 12c | 174307975 | 59 | 60 | CASPR4 |
| 12d | 174307979 | 61 | 62 | CASPR4 |
| 12e | 174307983 | 63 | 64 | CASPR4 |
| 12f | 174307987 | 65 | 66 | CASPR4 |
| 12g | 174307996 | 67 | 68 | CASPR4 |
| 12h | 169894929 | 69 | 70 | CASPR4 |
| 13a | CG59237-01 | 71 | 72 | 1 g, ring finger and |
| | | | | fibronectin domains |
| 13b | CG59237-02 | 73 | 74 | 1 g, ring finger and |
| | | | | fibronectin domains |
| 14a | CG58575-01 | 75 | 76 | phosphatidylserine synthase |
| | | | | 2-like |
| 15a | CG59256-01 | 77 | 78 | MHC class 1-like |
| 16a | CG59239-01 | 79 | 80 | MHC class 1-like |
| 17a | CG59295-01 | 81 | 82 | Otogelin-like |
| 18a | CG59293-01 | 83 | 84 | renal organic anion transport |
| | | | | protein 1-like |
| 19a | CG59284-01 | 85 | 86 | solute carrier family 22-like |
| 20a | CG59278-01 | 87 | 88 | GPCR P2-like |
| 21a | CG59274-01 | 89 | 90 | lipoma HMGIC fusion |
| | | | | partner-like |
| 21b | CG59274-02 | 91 | 92 | lipoma HMGIC fusion |
| | | | | partner-like |
| 22a | 172885510 | 93 | 94 | lipoma HMGIC fusion |
| | | | | partner-like |
| 23a | CG57734-01 | 95 | 96 | lipid associated protein-like |
| 23b | CG57734-02 | 97 | 98 | lipid associated protein-like |
| 23c | 198363601 | 99 | 100 | lipid associated protein-like |
| 24a | CG59389-01 | 101 | 102 | galactose binding lectin-like |
| 24b | CG59389-02 | 103 | 104 | galactose binding lectin-like |
| 24c | CG59389-04 | 105 | 106 | galactose binding lectin-like |
| 24d | 174308481 | 107 | 108 | galactose binding lectin-like |
| 24e | 174308497 | 109 | 110 | galactose binding lectin-like |
| 24f | 174308507 | 111 | 112 | galactose binding lectin-like |
| 24g | 174308517 | 113 | 114 | galactose binding lectin-like |
| 24h | 174308525 | 115 | 116 | galactose binding lectin-like |
| 25a | CG59885-01 | 117 | 118 | HGFR |
| 26a | CG93443-01 | 119 | 120 | LIV-1 |
| 27a | CG50838-01 | 121 | 122 | Leucine-rich repeat trans- |
| | | | | membrane protein FLRT3 |
| 28a | CG58567-01 | 123 | 124 | Protocadherin |
| 28b | CG58567-05 | 125 | 126 | Protocadherin |
| 28c | CG58567-06 | 127 | 128 | Protocadherin |
| 29a | CG59243-01 | 129 | 130 | Mitochondrial carrier-like |
| 29b | 188822080 | 131 | 132 | Mitochondrial carrier-like |
| 29c | CG59243-02 | 133 | 134 | Mitochondrial carrier like |
| 30a | CG59534-01 | 135 | 136 | membrane glycoprotein-like |
| 31a | CG59289-01 | 137 | 138 | Crumbs-like |
| 31b | CG59289-02 | 139 | 140 | Crumbs-like |
| 32a | CG57111-01 | 141 | 142 | Protocadherin 13-like |
| 33a | CG59363-01 | 143 | 144 | BAB26184-like |
| 33b | CG59363-02 | 145 | 146 | BAB26184-like |
| 33c | CG59363-03 | 147 | 148 | BAB26184-like |
| 34a | CG59301-01 | 149 | 150 | androgen receptor like |
| 35a | CG59525-01 | 151 | 152 | carcinoembryonic antigen |
| | | | | cgml-like |
| 36a | CG59484-01 | 153 | 154 | wd-repeat protein-like |
| 37a | CG57245-02 | 155 | 156 | CD40L Receptor Precursor- |
| | | | | like |
| 37b | CG57245-04 | 157 | 158 | CD40L Receptor Precursor- |
| | | | | like |
| 37c | 174308232 | 159 | 160 | CD40L Receptor Precursor- |
| | | | | like |
| 38a | CG59454-01 | 161 | 162 | Butyrophilin-like |
| 38b | CG59454-03 | 163 | 164 | Butyrophilin-like |
| 38c | CG59454-04 | 165 | 166 | Butyrophilin-like |
| 39a | CG59307-01 | 167 | 168 | DNA-binding protein-like |
| 40a | CG59713-01 | 169 | 170 | Van Gogh-like |
| 40b | 170645777 | 171 | 172 | Van Gogh-like |
| 41a | CG59570-01 | 173 | 174 | Aquaporin-like |
| 42a | CG56162-02 | 175 | 176 | Lysophospholipase-like |
| 42b | 174228465 | 177 | 178 | Lysophospholipase-like |
| 43a | CG59681-01 | 179 | 180 | immunoglobulin domain |
| | | | | containing protein |
| 43b | 174308213 | 181 | 182 | immunoglobulin domain |
| | | | | containing protein |
| 43c | 174308218 | 183 | 184 | immunoglobulin domain |
| | | | | containing protein |
| 43d | 174308224 | 185 | 186 | immunoglobulin domain |
| | | | | containing protein |
| 44a | CG59869-01 | 187 | 188 | Leucine rich repeat membrane |
| | | | | protein-like |
| 44b | CG59869-02 | 189 | 190 | Leucine rich repeat membrane |
| | | | | protein-like |
| 44c | CG59869-03 | 191 | 192 | Leucine rich repeat membrane |
| | | | | protein-like |
| 45a | CG59859-01 | 193 | 194 | Testis expressed protein 261 |
| | | | | (TEG-261)-like |
| 45b | CG59859-02 | 195 | 196 | Testis expressed protein 261 |
| | | | | (TEG-261)-like |
| 46a | CG59913-01 | 197 | 198 | ATP-binding cassette trans- |
| | | | | porter (ABC transporter)-like |
| 47a | CG59909-01 | 199 | 200 | ATP-binding cassette trans- |
| | | | | porter (ABC transporter)-like |
| 48a | CG59945-01 | 201 | 202 | Steroid Hormone Receptor- |
| | | | | like |

Table 1 indicates homology of NOVX nucleic acids to known protein families. Thus, the nucleic acids and polypeptides, antibodies and related compounds according to the invention corresponding to a NOVX as identified in column 1 of Table 1 will be useful in therapeutic and diagnostic applications implicated in, for example, pathologies and disorders associated with the known protein families identified in column 5 of Table 1.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in Column 5 of Table 1, the NOVX polypeptides of the present invention show homology to, and contain domains that are characteristic of other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table 1.

The NOVX nucleic acids and polypeptides are also useful for detecting specific cell types. Details of the expression analysis for each NOVX are presented in Example B. Accordingly, the NOVX nucleic acids, polypeptides, antibodies and related compounds according to the invention will have diagnostic and therapeutic applications in the detection of a variety of diseases with differential expression in normal vs. diseased tissues, e.g. a variety of cancers.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 101; (b) a variant of a mature form of the amino acid sequence selected from the group consisting of SEQ ID NO: 2n, wherein n is an integer between 1 and 101, wherein any amino acid in the mature form is changed to a different amino acid, provided that no more than 15% of the amino acid residues in the sequence of the mature form are so changed; (c) an amino acid sequence selected from the group consisting of SEQ ID NO: 2n, wherein n is an integer between 1 and 101; (d) a variant of the amino acid sequence selected from the group consisting of SEQ ID NO:2n, wherein n is an integer between 1 and 101 wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO: 2n, wherein n is an integer between 1 and 101; (b) a variant of a mature form of the amino acid sequence selected from the group consisting of SEQ ID NO: 2n, wherein n is an integer between 1 and 101 wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO: 2n, wherein n is an integer between 1 and 101; (d) a variant of the amino acid sequence selected from the group consisting of SEQ ID NO: 2n, wherein n is an integer between 1 and 101, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 101 or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n−1, wherein n is an integer between 1 and 101; (b) a nucleotide sequence wherein one or more nucleotides in the nucleotide sequence selected from the group consisting of SEQ ID NO: 2n−1, wherein n is an integer between 1 and 101 is changed from that selected from the group consisting of the chosen sequence to a different nucleotide provided that no more than 15% of the nucleotides are so changed; (c) a nucleic acid fragment of the sequence selected from the group consisting of SEQ ID NO: 2n−1, wherein n is an integer between 1 and 101; and (d) a nucleic acid fragment wherein one or more nucleotides in the nucleotide sequence selected from the group consisting of SEQ ID NO: 2n−1, wherein i is an integer between 1 and 101 is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNA's) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NO:2n−1, wherein n is an integer between 1-101, or a complement of this aforementioned nucleotide sequence, can be isolated using standard molecular biology techniques and the sequence information provided herein. Using all or a portion of the nucleic acid sequence of SEQ ID NO:2n−1, wherein n is an integer between 1-101, as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO:2n−1, wherein n is an integer between 1-101, or a complement thereof. Oligonucleotides may be chemically synthesized and may also be used as probes.

In another embodiment, an isolated nucleic acid molecule of the invention comprises a nucleic acid molecule that is a complement of the nucleotide sequence SEQ ID NO:2n−1, wherein n is an integer between 1-101, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NO:2n−1, wherein n is an integer between 1-101, is one that is sufficiently complementary to the nucleotide sequence of SEQ ID NO:2n−1, wherein n is an integer between 1-101, that it can hydrogen bond with little or no mismatches to the nucleotide sequence of SEQ ID NO:2n−1, wherein n is an integer between 1-101, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to all aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO:2n−1, wherein n is an integer between 1-101, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:2n−1, wherein n is an integer between 1-101; or an anti-sense strand nucleotide sequence of SEQ ID NO:2n−1, wherein n is an integer between 1-101; or of a naturally occurring mutant of SEQ ID NO:2n−1, wherein n is an integer between 1-101.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO:2n−1, wherein n is an integer between 1-101, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences of SEQ ID NO:2n−1, wherein n is an integer between 1-101, due to degeneracy of the genetic code and thus encode the same NOVX proteins as that encoded by the nucleotide sequences of SEQ ID NO:2n−1, wherein n is an integer between 1-101. In another embodiment, an isolated nucleic acid molecule of the invention has a nucleotide sequence encoding a protein having an amino acid sequence of SEQ ID NO:2n, wherein n is an integer between 1-101.

In addition to the human NOVX nucleotide sequences of SEQ ID NO:2n−1, wherein n is an integer between 1-101, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from any one of the human SEQ ID NO:2n−1, wherein n is an integer between 1-101, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:2n−1, wherein n is an integer between 1-101. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, New York (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6× SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2× SSC, 0.01% BSA at 50° C. An isolated nucleic acid molecule of the invention that hybridizes under stringent conditions to any one of the sequences of SEQ ID NO:2n−1, wherein n is an integer between 1-101, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:2n−1, wherein n is an integer between 1-101, or fragments, analogs or derivatives thereof, under conditions of moderate stringency is provided. A non-limiting example of moderate stringency hybridization conditions are hybridization in 6× SSC, 5× Reinhardt's solution, 0.5% SDS and 100 mg/ml denatured salmon sperm DNA at 55° C., followed by one or more washes in 1× SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, New York, and Krieger, 1990; GENE TRANSFER AND EXPRESSION, A LABORATORY MANUAL, Stockton Press, New York.

In a third embodiment, a nucleic acid that is hybridizable to the nucleic acid molecule comprising the nucleotide sequences of SEQ ID NO:2n−1, wherein n is an integer between 1-101, or fragments, analogs or derivatives thereof, under conditions of low stringency, is provided. A non-limiting example of low stringency hybridization conditions are hybridization in 35% formamide, 5× SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/volt) dextran sulfate at 40° C., followed by one or more washes in 2× SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, New York, and Kriegler, 1990, GENE TRANSFER AND EXPRESSION, A LABORATORY MANUAL, Stockton Press, New York; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NO:2n−1, wherein n is an integer between 1-101, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence of SEQ ID NO:2n, wherein n is an integer between 1-101. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from any one of SEQ ID NO:2n−1, wherein n is an integer between 1-101, yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NO:2n, wherein n is an integer between 1-101. Preferably, the protein encoded by the nucleic acid molecule is at least about 60% homologous to SEQ ID NO:2n, wherein n is an integer between 1-101; more preferably at least about 70% homologous to SEQ ID NO:2n, wherein n is an integer between 1-101; still more preferably at least about 80% homologous to SEQ ID NO:2n, wherein n is an integer between 1-101; even more preferably at least about 90% homologous to SEQ ID NO:2n, wherein n is an integer between 1-101; and most preferably at least about 95% homologous to SEQ ID NO:2n, wherein n is an integer between 1-101.

An isolated nucleic acid molecule encoding a NOVX protein homologous to the protein of SEQ ID NO:2n, wherein n is an integer between 1-101, can be created by introducing one or more nucleotide substitutions, additions or deletions into the nucleotide sequence of SEQ ID NO:2n−1, wherein n is an integer between 1-101, such that one or more amino acid substitutions, additions or deletions are introduced into the encoded protein.

Mutations can be introduced into any of SEQ ID NO:2n−1, wherein n is an integer between 1-101, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of any one of SEQ ID NO:2n−1, wherein n is an integer between 1-101, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NO:2n−1, wherein n is an integer between 1-101, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO:2n, wherein n is an integer between 1-101, or antisense nucleic acids complementary to a NOVX nucleic acid sequence of SEQ ID NO:2n−1, wherein n is an integer between 1-101, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyl adenine, 1-methylguanine, 1-methylinosine 2,2-dimethylguanine, 2-methyladenine, 2-methylguanine, 3-methylcytosine, 5-methylcytosine, N6-adenine, 7-methylguanine, 5-methylaminoethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, el al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See. e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., any one of SEQ ID NO:2n−1, wherein n is an integer between 1-101). For example, a derivative of a Tetrahymena L-19 IVS RNA can be constructed in which the nucleotide any of whose residues may be changed from the corresponding residues shown in any one of SEQ ID NO:2n, wherein n is an integer between 1-101, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NO:2n, wherein n is an integer between 1-101. The invention also includes a mutant or variant protein about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO:2n, wherein n is an integer between 1-101) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO:2n, wherein n is an integer between 1-101. In other embodiments, the NOVX protein is substantially homologous to SEQ ID NO:2n, wherein n is an integer between 1-1 01, and retains the functional activity of the protein of SEQ ID NO:2n, wherein n is an integer between 1-101, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO:2n, wherein n is an integer between 1-101, and retains the functional activity of the NOVX proteins of SEQ ID NO:2n, wherein n is an integer between 1-101.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO:2n−1, wherein n is an integer between 1-101.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO:2n, wherein n is an integer between 1-101, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences thereini. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

NOVX Antibodies

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO:2n, wherein n is an integer between 1-101, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is ≦1 μM, preferably ≦100 nM, more preferably ≦10 nM, and most preferably ≦100 pM to about 1 pM, as measured by assays such as radioligand binding assays or similar assays known to those skilled in the art.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, New York, incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polygonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells arc cultured can then be assayed for the presence of monoclonal antibodies directed against the antigen. Preferably, the binding specificity of monoclonal antibodies produced by the hybridoma cells is determined by immunoprecipitation or by an in vitro binding assay, Such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Techology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies arc also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxinis can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Designs 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas acruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as gluitareldehlyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepenitaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77:4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al ., J. Biol. Chem., 257: 286-288 (1982) via a disulfide-interchange reaction. A chemotherapeutic agent (such as Doxorubicin) is optionally contained within the liposome. See Gabizon ct al., J. National Cancer Inst., 81(19): 1484 (1989).

Diagnostic Applications of Antibodies Directed Against the Proteins of the Invention

Antibodies directed against a protein of the invention may be used in methods known within the art relating to the localization and/or quantitation of the protein (e.g., for use in measuring levels of the protein within appropriate physiological samples, for use in diagnostic methods, for use in imaging the protein, and the like). In a given embodiment, antibodies against the proteins, or derivatives, fragments, analogs or homologs thereof, that contain the antigen binding domain, are utilized as pharmacologically-active compounds (see below).

An antibody specific for a protein of the invention can be used to isolate the protein by standard techniques, such as immunoaffinity chromatography or immunoprecipitation. Such an antibody can facilitate the purification of the natural protein antigen from cells and of recombinantly produced antigen expressed in host cells. Moreover, such an antibody can be used to detect the antigenic protein (e.g., in a cellular lysate or cell supernatant) in order to evaluate the abundance and pattern of expression of the antigenic protein. Antibodies directed against the protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thius the receptor mediates a signal transduction pathway for which ligand is responsible.

Alteratively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polygonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labelinig of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Thory of Enzyme Immunloassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylationl signals). Such regulatory sequences are described, for example, in Coeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathionc S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF89 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO. J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO. J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genies see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human, animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences, i.e., any one of SEQ ID NO:2n−1, wherein n is an integer between 1-101, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191 ; and Hogan, 1986. In: MANIPULATING THE MOUSE EMBRYO, Cold Spring Harbor Laboratory Press, Cold Spring Harbor, N.Y. Similar methods arc used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NO:2n−1, wherein n is an integer between 1-101), but more preferably, is a non-human homologue of a human NOVX gene. For example, a mouse homologue of human NOVX gene of SEQ ID NO:2n−1, wherein n is an integer between 1-101, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage PI. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compound(s can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabeins; anitioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses antimicrobial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomlimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. USA. 90: 6909; Erb, et al., 1994. Proc. Natl. Acad. Sci. USA. 91: 11422; Zuckermann, et al., 1994.,J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with i test compound and the ability of the test compound to bind to a NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. A NOVX target molecule can be a non-NOVX molecule or a NOVX protein or polypeptide of the invention. In one embodiment, a NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1 1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NFIS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchii, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences of SEQ ID NO:2n−1, wherein n is an integer between 1-101, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, el al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected inindividuals, then the mutation is likely to be the causative agent of the particular disease. Comparison of affected and unaffected individuals generally involves first looking for structural alterations in the chromosomes, such as deletions or translocations that are visible from chromosome spreads or delectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NO:2n−1, wherein n is an integer between 1-101, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO:2n−1, wherein n is an integer between 1-101, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genome DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g, Landegran, et al., 1988. Science 241:1077-1080; and Nakazawa, et al., 1994. Proc. Natl. Acad Sci. USA 91: 360-364), the latter of which can be particularly useful for detecting point mutations in the NOVX-gene (see, Abravaya, et al., 1995. Nucl. Acids Res. 23:675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87:1874-1878), transcriptional amplification system (see, Kwoh, et al., 1989. Proc. Natl. Acad. Sci. USA 86:1173-1177); Qβ Replicase (see, Lizardi, et al, 1988. BioTechnology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. .Sci. USA 74: 560 or Sanger, 1977. Proc. Natl. Acad. Sci. USA 74: 5463. It is also contemplate(d that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation. polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7:5.

In yet another embodiment, the movement of mutant or wild-type fragments in polyacrylanide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6:1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochhrome PREGNANCY ZONE PROTEIN PRECURSOR enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard closes. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) oil the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markets of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministrational sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Osteodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244:1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity has a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

### EXAMPLES

### Example A

Polynucleotide and Polypeptide Sequences, and Homology Data

### Example 1

The NOV1 clone was analyzed, and the nucleotide and polypeptide sequences are shown in Table 1A.

TABLE 1A
NOV1 Sequence Analysis

| | |
|---|---|
| SEQ ID NO: 1 | 8554bp |

| | |
|---|---|
| NOV1a | GCACCCCGACAAGATGCCCAAGCGCGCGCACTGGGGGGCCCTCTCTGTGGTGCTGATC |
| CG58546-01 DNA | |
| Sequence | CTGCTTTGGGGTCTCCGCGAGTGGCGCTGGCCTGCCCTCATCCTTGTGCCTGCTACG |
| | TCCCCAGCGAGGTCCACTGCACGTTCCGATCCCTGGCTTCTGTGCCCGCTGGCATTGC |
| | TAAACACTGTGGAAAGAATCAATTTGGGGTTTGGAAATAGCATACAGGCCCTGTCAGAA |
| | ACCTCATTTGCAGGACTGACCAAGTTGGAGCTACTTATGATTCACGGCAATGAGATCC |
| | CAAGCATCCCCGATGGAGCTTTAAGAGACCTCAGCTCTCTTCAGGTTTTCAAGTTCAG |
| | CTACAACAAGCTGACAGTGATCACAGGACAGACCCTCCAGCGTCTCTCTAACTTAATG |
| | AGGCTGCACATTGACCACAACAAGATCGAGTTTATCCACCCTCAAGCTTTCAACGGCT |
| | TAACCTCTCTGAGGCTACTCCATTTGGAAGGAAATCTCCTCCACCAGCTGCACCCCAG |
| | CACCTTCTCCACGTTCACATTTTTGGATTATTTCAGACTCTCCACCATAAGGCACCTC |
| | TACTTAGCAGAGAACATGGTTAGAACTCTTCCTGCCAGCATGCTTCGGAACATGCCGC |
| | TTCTGGAGAATCTTTACTTGCAGGGAAATCCGTGGACCTGCGATTGTGAGATGAGATG |
| | GTTTTTGGAATGGGATGCAAAATCCAGAGGAATTCTGAAGTGTAAAAGGACAAAGCT |
| | TATGAAGGCGGTCAGTTGTGTGCAATGTGCTTCAGTCCAAAGAAGTTGTACAAACATG |
| | AGATTCACAAGCTGAAGGACCTGACTTGTCTGAAGCCTTCCATAGAGTCTCCTCTGAG |
| | ACAGAACAGGAGCACGAGTATTGAGGAGGAGCAACAAGAAGAGAATGGTGACAGC |
| | CAGCTCATCCTGGAGAAAATCCAACTTCCCCAGTGGAGCATCTCTTTGATATGACTG |
| | ATGAGCACGGGAACCTGGTGAACTTGGTGTGTGACATCAAGAAACCAATGGATGTGTA |
| | CAAATTCACTTGAACCAAACAGATCCTCCAGATATTGACATAAATGCAATGGTTGCC |
| | TTGGACTTTGAGTATCCAATGACCCAGGAAAACTATGAAAATCTATGGAAATTGATAG |
| | CATACTACAGTGAAGTTCCCATGAAGCTACACAGAGAGCTCATGCTCAGCAAACACCC |
| | CAGAGTCAGCTACCAGTACAGGCAAGATGCCGATCAAGAAGCTCTTTACTACACAGGT |
| | GTGAGAGCCCAGATTCTTGCAGAACCAGAATGGATCATGCAGCCATCCATAGATATCC |
| | AGCTGAACCGACCTCAGAGTACCCCCAGAJGGTGCTACTTTCCTACTACAACCAGTA |
| | TTCTCAAACAATAGCCACCAAGATACAGGCAGGCTCGGGGCAGAAGCTGGGTAATG |
| | ATTGAGCCTAGTAGAGCTGTGCAAAAAGATCAGACTGTCCTGGAAGGGGGTCGATGCC |
| | AGTTGAGCTGCAATGTGAAACCTTCTGAGAGTCCATCTATCTTCTGGGTGCTTCCAGA |
| | TGGCTCCATCCTGAAAGTGCCTGTCGATGACCCAGACAGCAAGTTCTCCATTCTCAGC |
| | AGTGGCTGCCTGAGGATCAAGTCCATGCAAGCCATCTGACTCGGGCTTCTACCAGTGCA |
| | TTGCTCAAGTGAGGGATGAAATGGACCGCATGGTATATAGGGTACTTGTGCAGTCTCC |
| | CTCCACTCAGCCAGCCGAGAAGACACAGTGACAAATTGGCAAGAACCCAGGGGAGCCA |
| | GTGATGTTGCCTTGCAATGCTTTAGCTATACCCGAAGCCCACCTTAGCTGGATTCTTC |
| | CAACAGAAGGATAATTAATGATTTGGCTAACACATCACATGTATACATGCTGCCAAA |
| | TGGAACTCTTTCCATCCCAAAGGTCCAAGTCAGTGACAGTGGTTACCACAGATGTGTG |
| | GCTGTCAACCAGCATGGGGCAGACCATATCACGGTGGGAATCACAGTCACCAAGAAAG |
| | GTTCTGGCTCGCCATCCAAAAGAGGCAGATGGCCAGGTCCAAAGGCTCTTTCCAGATC |
| | CAAAGGCTCTTTCCAGATGAGAGAAGACATCGTGGACCATGAAGGGGTCTCAGCCACG |
| | CGAGATGAAGAGAACACTTCAAGGAGACTTCTACATCCAAAGCACCAAGAGGCGTTCC |
| | TCAAAACAAAGGATGATGCCATCAATGGAGATAAGAGCCAAGAAAGGGAGAAGAAA |
| | GCTCAAACTCTGGAAGCATTCACAAAAGAACCAGAGACCACTGTTCCAGAAGATCTC |
| | ACTGGGCTGATATTTT AGAGTGTTTGAATCAAGACGAAGGATAAACGTGGCAAACAAACAGATTAATCCGGAGC |
| | ATCCCCAATTATTAAAAGCCAAAGTCTTTGGGAAAAATCTCCCTACAGGCACAGAAGT |
| | TTGCCTGCTGTTTCTCACCACAAGTTCTCCATTCTTGAGCCTAGTAGTCACACCACCT |
| | AATCCTCAGCAGATGTCCCCCTTGGCATCTCCAATACAGACAGCAAACAAGTGCTGAAC |
| | CTCAGCCAGCATGGGAACCTCTACTCAGCGAAGGAAAGCACATTTTGAGTACCATTTC |
| | GAACAAGCACACCTCTCTAGAACACCACAACAITGGAGTTATTCTTGTTGAACCTGAAX |
| | TCACTTCCACTGAAGGCGAAGAAGTTGTTGATGAGTATTCCAAGAAGACTGAGGAGA |
| | TTATGAACAATCTCCTCGACCTGAAGGGCACTGCAGCCTCTACACTTATATCTGAGCC |
| | GAAGAGACGGAAACAGACTCTACACACCTTAGACACAGTCTATGAAGAGCCCACCCAT |
| | CATCCAGTGAGTATGAAGGGTTGGTCTGCAGCAGATGTTGGATCCTCACCAGATCCCA |
| | GCAATACTTTGACCCAGCTTCCATTGGTTGTTGTCTCCTTCCCTGAGTCTAGCCTGT |
| | GAATACAGTTTTGCACGATTTGGAGACTAATTCACAACCACATGAGGATAACATAAAA |
| | CATCACTGTCATTTGAACCTTACTCCAACCGCCATCATCTGGTTTAATGACTCTAGTA |
| | TCTACAAGGACCGACAGGATTCTACTGTAGGGGAACAAGGTGTCCCAGGCAAATCACA |
| | ACCTTATTGATTAAAAGAGAACATCCAGCTTGTGAAAAGTAGTTTTAGCACTCAAGAC |
| | TGCTAGAGGGAGACCCAACGTATGAAAGAGATGTCTCAGACACTACAGGGACGATA |
| | ATCCATCACTTTACCTTACACACTCCAGAAGTTCTGAGATGAGGGCCAAGAGAGCAA |
| | AAGCCTGCGGAAACCAGACTCCACACTGGGTATAACCAGCAGTACGTCTCCAGTTAAG |
| | CTCCAAGGCAAAAAGTCAGTTGTCACCTGCTACACAAAGACACCACAACAGAAACAA |
| | ACCCAATGGGAGAAAAGGCTTCATCATCCACCATGAGCACTCACCCTTCTCGAAGAAA |
| | CCCACAACTTTTGCTCTTACACCCTCACAAATTCCACCACCCGACAAGCAAACCCCA |
| | TTAAGATTTCAAATCACATTAGAGACTTTTTCTACTCACCACTCAACCAACTGACA |
| | AGTTAATACCCCCAAAAATGGAGAGTTCTCTGGTTCCTACATCTTGGGACATTAACAC |
| | ACTCCACGGAGAAAACCAGCTGGMATGGAGAAGAATGTACAGCTCATATCAAAAGCAA |
| | TGAGTTCAAGAGCATCACGGGAAGAGGCCAAACAAACATCGATATACCCCTTCTACAG |
| | CATTGTTACTCCCAGTTGCATCCAAGCCCAGCCCTTCTCCAGATAACATAGAA |
| | GAGGGCGTTTATGATTTCAGAAACTACACTTTTGCCTAGAAATGTTTCTCTGAAAACT |
| | ACCATAAAGTCCAAGACCTTAGATTACACGACAACCACCAGAAAAATACATTCATCTC |
| | AATTCAGGATGATGTTCACACTTCCAGTCATGTATAAACCCACATCAGATGGAAAAGA |
| | GGTGAGTCAATTACAAGCCACAAATGTTGACAAACATAAAAGTGACATTTTAGTCCCT |
| | AATTTAAGGAAGAATCATGTCACACkACTTCTCGCTCCTTGGTCTCCACTATGGGAG |
| | AAGGAAAGCTCAGCCTCTCTCCTGTGGGCTTTCCAGGAATTCCAACCTGGAATCCCTC |
| | ACCCCTACAGACCCTCGGGAGGCTACAGACAGACATACATGTTACCACTTCTGGGGAA |
| | TTTTGTCCTCTGTGACCCCTTGTTAACGAGCTTGAGGATGTGGATTTTACTTCTGAGT |
| | AATTCTCTCAAGCATAAGTCTCCACACCATTTCACCAGGAAGAAGCTGGTTTTTCCAC |
| | GGTCAAGATCATCATGAAAGTGGAGATGGCTTCAAGTCACGTAGAAACTACCACCCTT |
| | ATCACATCCTTACTGCAAACCACTGTGGCTATTCTCCACTCTGAAACTAGACCACAGA |
| | TCTCCTGTCTTTGGGATGCCTGGATGAAGGAGCCAGCATCTTTGTCCCCTCCCATGAT |
| | TCTCAAATATGTAAAGCAAACCACCACCACTAAGCCAGAACTTCTCAGTCCAAGAACA |
| | AAACAGAAGCAACCCCATTCCAAGGAAAATGTTTTCTTGAATTACATGGGGAAACCAG |
| | ATTATCAACACCATCTAGTGAAAATGAAGGAACACAGCGTATGTCAGGGCCAAATGA |
| | GAAAAGCAAGTATTTGTCTGACCACGATGCATTTAACTTGTCTACAAAGCTAGAATTG |
| | ATGGAAGAGTTCATGCATAGTAGGAGTCTAACACGTGGCCCACATAGCCACCACCAGG |
| | AACAGGAACAGTGAGGTTCTCATCAACTAACCAGAATCCCTGCCAAACCCATCCTACC |
| | ACTTTCCAGCCACCTCCTGCCTGAAATGTCCACACAAAGCACTTCCAGATACTTTGTA |
| | GGGCTTTGCCAGAGAGATCACGGGACCAACAAACCAGAAATAACTACATATCCTTCTA |
| | CCTATCACACATGTCCCAACAGTTTACAACTCCAAGAGTAGCAAGTACACTCCTCT |
| | CAATCCAATGGCTCCTAACCCAGCATTTCTAGTAAGTTTCCTGACCTAAGAACTGAC |
| | CAGTTGGAAAGCCTCTACAAAGTGTTTGGAAATAGCAACATCCCTGAGGCAAGAAACT |
| | CTTTACCAACAGGACTCAGTCCAAGAATTTATCATTATTCCAATGGAAGACTCCCTTT |
| | CCCTCTTCTCCTGTCCCTTTCTTTTTCACAGTTGGGAGTCACCCGGAGACCCCAGATA |
| | GGATATATTCCCATAGCAGTAATGAGAGAGAGAAAAGTTAATCCAGGTTCCTACAATA |
| | GCACACTCCATGGACCCACCTTCCATCTGGACTTTGGCCTTCCAGCACCTCCACTGTT |
| | TCATCCACCCAGAGTTATGGTATCACCCCCAACTAACTTACAGAATATCCCTATGGTC |
| | TCCACCAAAGCGGCTCCTGTCTCCTTTATAACATCTTCTGTCCAGTCCTCAGGAAGCA |
| | TCTTGGGCAAAAGCCCAAAGTTCTTTGCAGGAGGACCGCCTGCATCCAAATTCTGGCC |
| | CAAATCCTCACCAAGTCCCCACAGACTGTGTCTGTCACTGCT |
| | GAAACGGACGCTGTGTTCCCGTGTGAGGCAATAGGAACCAAAAGCCTTTCGTTACTT |
| | GGACAAAAGTTTCCACATCTCCAGGAGTTCTTATGACTCCGAATACCAGGATACAACG |
| | GTTTGAGGTTCTCAAGAACCGTACCTTAGTGATAAGGAAGTTTCAAGTGCAAGATCGA |
| | GGCCAGTATATGTGCACCGCCAGCAACCTGTACGGCCTGGACAGGATGGTGGTCTTTC |
| | TCTGGGTCACCGTGCAGCAACCTCAAATCCTAGCCTCCCACTACCAGGACGTCACCGT |
| | CTACCTGGGAGACACCATTACAATGGAGTGTCTGGCGAAAGGGACCCCAGCCCCCCAAA |
| | ATTTCCTGGATCTTCCGTGACAGGAGGGTGTGGCAAACTCTGTCCTCCGTGGAGGGCC |
| | GGATCACCCTGCACCAAAACCGGACCCTTTCCATCAAGGAGGCGTCCTTCTCAGACAG |
| | AGGCGTCTATAAGTGCGTGGCCAGCAACGCAACCCGGGCGGACAGCGTGTCCATCCGC |
| | CTACACGTGGCGGCACTGCCCCCCATTATCCACCAGGAGAAGCTGGAGAACATCTCGC |
| | TGCCCCCGGGGCTCAGCATTCACATTCACTGCACTGCCAAAGCTGCGCCCCTGCCCAG |
| | CGTGCTCTGGGTGCTCGGGGATGCATACCCAAATCCGCCCCTCGCATTTCCTCCACCGG |
| | AACTTGTTTGTTTTCCCCAACGGGACGCTCTACATCTGCAACCTCGCGCCCAAGAAAA |
| | GCGGGCGCTATGAGTGCGTGGCCGCCAACCTGATCGGCTCCGCGCGCAGTACGGTGCA |
| | GCTGAACGTGCAGCGCGCAGCAGCGAACGTGCAGCGCGCAGCAGCGAACGTGCAGCGC |
| | GCCAACGCGCGCATCACGGGCACCTCCTCGCAGAGGACGGACGTCAGCTACGGAGGGA |
| | CCCTCAAGCTGGACTGCAGCGCCTCGGGGGATCCCTGGCCGCGCATCCTCTGGAGGCT |
| | GCCGTCCAAGAGGACGATCGACGCGCTTTTCAGTTTTGATAGTAGAATCAAGGTGTTT |
| | GCCAACAGGACCCTGGTGGTGAAATCAATGACAGACAAAGACGCCGGAGATTACCTGT |
| | GTGTAGCTCGAATAAGCTTCGTGATGACTGCGTGGTGCTCAGGTGGATGTGATGAT |
| | GAAACCGGCCAAGATTGAACACAAGGAGGAGAACGACCACAPAAGTCTTCTACAGGGGT |
| | GACCTCAAAGTGGACTGTGTGGCCACTGCACTTCCCAITCCCGAGATCTCCTGGAGCC |
| | TCCTGGATGCGAGTCTGGTGAACTCCTTCATCCAGTCAGATGACAGTGGTGGACCCAC |
| | CAAGCACTATGTGGTCTTCAACAATGGGACACTCTACTTCAGTGAAGTGGGGATGAGG |
| | GAGGAAGGAGACTACACCTGCTTTGCTGAAAATCAGGTTGGGAAGGATGAGATGAGAG |
| | TCAGAGTCAAGATGGTGACACCTGCCACCATCTGGAACAAGACTTACTTGGCAGTTCA |
| | GGTACCCTATGGAGATCTGGTCACTGTAACCTGTGAGGCCAAACGAGAACCCATGCCC |
| | AAGGTGACTTGGTTGTCCCCAGCCAACAGGGTGATCCCCACCTCCTCTGAGAAAGTATC |
| | AGATATACCAATATGGCACTCTCCTTATTCAGAAAGCCCAGTGCTCTGACAGCGGCPA |
| | CTACACCTGCCTGGTCAGGAACAGTGCCGGAGAGGATACGAAGACAGTGTGGATTCAC |
| | GTCAACCTCCAGCCACCCAAGATCAATGGTAACCCCAACCCCATCACCACCGTGTGGG |
| | AGATAGCAGCCGCGGCCAGTCGGAAACTGATTGACTGCAAAGCTGAAGGCATCCCCAC |
| | CCCGAGGGTGTTATGGGCTTTTCCCCAGGGTGTGGTTCTGCCAGATCCATACTATGGA |
| | AACCGGATCACTGTCCATGGCAACGGTTCCCTGCACATCAGGAGTTTGAGGAAGAGCG |
| | ACTCCGTCCAGCTGGTATGCATGGCACGCAACGAGGGAGCGGAGGCGAGGTTGATCGT |
| | GCAGCTCACTGTCCTGGAGCCCATGGAGAAACCCATCTTCCACCACCCGATCAGCGAG |
| | AAGATCACGGCCATGGCGGGCCACACCATCACCCTCAACTGCTCTGCCGCGGGGACCC |
| | TGACACCCAGCCTGGTGTGGGTCCTTCCCAATGGCACCGATCTGCAGAGTGGACAGCA |
| | GCTGCAGCGCTTCTACCACAAGGCTGACGGCATGCTACACATTAGCGGTCTCTCCTCG |
| | GTGGACGCCGGGGCCTACCGCTGCGTGGCCCGCAATGCCGCGGCCCACACGGAGAGGC |
| | TGGTCTCCCTGAAGGTGGGACTGAAGCCAGAAGCAAACAAGCAGTATCATAACCTGGT |
| | CAGCATCATCAATGGTGAGACCCTGAAGCTCCCCTGCACCCCTCCTGCAGCTGGGCAG |
| | GGACATTTCTCCTGGACACTCCCCAATGGCATGCATCTOGAGGGCCCCCAAACCCTGG |
| | GACGCGTTTCTCTTCTCGACAATGGCACCCTCACGGTTCGTGAGGCCTCGGTGTTTGA |
| | CAGCGGTACCTATGTATGCAGCATGGAGACGGCGTACGGCCCTTCCGTCACCAGCATC |
| | CCCGTGATTGTCATCGCCTATCCTCCCCGGATCACCAGCGAGCCTACCCCAGTCATCT |
| | ACACCCGTCCCCGGAACACCGTGAACTGAACTGCATGGCTATGGGGATTCCCAAAGG |
| | TGACATCACGTGGGAGTTACCGGATAAGTTGCATCTGAACGCAGGGGTTCAGGCTCGT |
| | CTGTATGGAAACAGATTTCTTCACCCCCAGGGATCACTGACCATCCAGCAGGCCAGAC |
| | CGAGACACGCTGGCTTCTACAAGTGCACGGCAAAAAACATTCTCAGCAGTGACTCCAA |
| | AACAACTTATATCCATGTCTTCTGAAAT |

| | |
|---|---|
| ORE Start: ATG at 14 | ORE Stop: TGA at 8549 |

| | | |
|---|---|---|
| SEQ ID NO: 2 | 2845 aa | MW at 31 5664.5kD |

| | |
|---|---|
| NOV 1a, | MPKRAHWGALSVVLILLWGHPRVALACPHPCACYVPSEVHCTFRSLASVPAGIAKHVE |
| CG58546-01 Protein | |
| Sequence | RINLGFGNSIQALSETSFAGLTKLELLMIHGNEIPSIPDGALRDLSSLQVFKSYNKL |
| | RVITGQTLQGLSNLMRLHIDHNKIEFIHPQAFNGLTSLRLLHLEGNLLHQLHPSTFST |
| | FTFLDYFRLSTIRHLYLAENMVRTLPASMLRNMPLLENLYLQGNPWTCDCEMRWFLEW |
| | DAKSRGILKCKKDKAYEGGQLCAMCFSPKKLYKHETHKLKDLTCLKPSIESPLRQNRS |
| | RSIEEEQKQEENGDSQLILEKIQLPQWSISLNMTDEHGNLVNLVCDIKKPMDVYKIHL |
| | NQTDPPDIDINAMVALDFEYPMTQENYENLWKLIAYYSEVPMKLHRELMLSKIPRVSY |
| | QYRQDADEEALYYTGVRAQILAEPEWIMQPSIDIQLNRPQSTAKKVLLSYYNQYSQTI |
| | ATKDTRQARGRSWVMIEPSRAVQKDTVLEGGRCQLSCVKASESPSIFWVLPDGSIL |
| | KVPVDDPDSKFSILSSGWLRIKSMEPSDGLYQCIAQVRDEMDRVYRVLVQSPSTQP |
| | AEKDTVTIGKNPGEPVMLPCNALAIPEAHLWILPNRRIINDLANTSHVYMLPNGTLS |
| | IPKVQVSDSYHRCVAVNQHGADHITVGITVTKKGSGSPSKRGRWPGPKALSRSKGSF |
| | QMREDIVEDEGVSGTGDEENTSRRLLHPHQEAFLKTKDDAINGDKKAKKGRRKLKLW |
| | KHSEKEPETSVAEDLRVFESRRRINVANKQINPEHWADILAKVFGKNLPTGTEVSPII |
| | KTTSSPFLSLVVTPPLPAVSPPLASPIQTATSAEESSADVPLLSEGKHILSTISSASM |
| | GLEHHNNGVILVEPEVTSTPLEEVVDEYSKKTEEMTSTEGDLKGTAASTLISEPYEQS |
| | PTLHTLDTVYEEPTHEETETEGWSAADVGSSPDPTSSEYELPLVVVSLAESKPVQYFD |
| | PDLETNSQPHEDNIKEYSFAHLTPTAIIWFNDSSTSLSFEDSTVGEQGVPGKSHLQGP |
| | TENIQLVKSSFSTQDTLLIKKGMKEMSQTLQGGNMLEGDPTHSRSSENEGQESKSITL |
| | PDSTLGITSSTSPVKKPAETTVVTLLHKDTTTETTPRQKVASSSTMSTHPSRRRPNGR |
| | KLHPHKFHHRHKQTPPTTFAPLETFSTQPTQATDIKISNQMESSLVPTWEINTVNTP |
| | KQLEMEKNVELISKGTPPPKHGKRPNKHRYTPSTVSSRASASKPSPSPENKHRNIVTP |
| | SSETTLLPRNVSLKTEGVYDSLDYTTTTRKIHSSHHKVQDTLPVMYKPTSDGKEIQDD |
| | VATNVDKHKSDILVPGESITNVTQTSRSLVSTMGEFKEESSPVGFGIPTWNPSRKAQ |
| | PGRLQTDIHVTTSGETPTDPPLVNELEDVDFTSEFLSSVTVSTPFHQEEAGFSTILSS |
| | IKVEMASSQVETTTLGQDHHETTVAILHSETRPQNHILTAAWMKEPASLSPPMILLSL |
| | GQTTTTKPELLSRTSQICKDSKENVFLNYMGNPETEATPVKEGTQRMSGPNELSTP |
| HVF | |

| | |
|---|---|
| SEQ ID NO: 3 | 762 bp |

| | |
|---|---|
| NOV1b, | CGGCCGTGCCCTCATCCTTGTGCCTGCTACGTCCCCAGCOAGGTCCACTGCACGTTCC |
| 174307918 DNA | |
| Sequence | GATCCCTGGCTTCCGTGCCCGCTGGCATTGCTAACACGTGGAAAAGAATCAATTTGGG |
| | GTTTAATAGCATACAGGCCCTGTCAGAAACCTCATTTGCAGGACTGACCAAGTTGCAG |
| | CTACTTATGATTCACGGCAATGAGATCCCAAGCATCCCCGATGGAGCTTTAAGAGACC |
| | TCAGCTCTCTTCAGGTTTTCAAGTTCAGCTACAACAAGCTGAGAGTGATCACAGGACA |
| GACCCTCCAGGGTCTCTCTAACTTAATGAGGCTGCACATTGACCACAACAAGATCGAG | |
| | TTTATCCACCCTCAAGCTTTCACGGCTTAACGTCTCTGAGGCTACTCCATTTGGAAG |
| | GAAATCTCCTCCACCAGCTGCACCCCAGCACCTTCTCCACGTTCACATTTTTGGATTA |
| | TTTCAGACTCTCCACCATAAGGCACCTCTACTTCGCAGAGAAACATGGTTAGAACTCTT |
| | CCTGCCAGCATGCTTCGGAACATGCCGCTTCTGGAGAATCTTTACTTGCAGGGAAATC |
| | CGTGGACCTGCGATTGTGAGATGAGATGGTTTTTGGAATGGGATGCAAAATCCAGAGG |
| | AATTCTGAAGTGTAAAAAGGACAAAGCTTATGAAGGCGGTCAGTTGTGTGCAATGTGC |
| | TTCAGTCCAAAGAAGTTGTACAAACATGAGATTCACAAGCTGAAGGACCTGACTTGTC |
| | TGCTCGAG |

| | |
|---|---|
| ORF Start: CGG at 1 | ORF Stop: it at 763 |

| | | |
|---|---|---|
| SEQ ID NO: 4 | 1254 aa | MW at 29088.6kD |

| | |
|---|---|
| NOV 1b, | RPCPHPCACYVPSEVIICTFRSLASVPAGIAKHVERINLGFNSIQALSETSFAGLTKLE |
| 174307918 Protein | |
| Sequence LLMTHGNEIPSIPDGALRDLSSLQVFKFSYNKLRVITGQTLQGLSNLMRLHIDHNKIE | |
| | FIHPQAFNGLTSLRLLHLEGNLLHQLHPSTFSTFTFLDYFRLSTIRHLYFAENMVRTL |
| | PASMLRNMPLLENLYLQGNPWTCDCEMRWFLEWDAKSRIGILKCKKDKAYEGGQLCAMC |
| | FSPKKLYKHEIHKLKDLTCLLE |

| | |
|---|---|
| SEQ ID NO: 5 | 762 bp |

| | |
|---|---|
| NOV 1c, | CGGCCGTGCCCTCATCCTTGTGCCTGCTACGTCCCCAGCGAGGTCCACTGCACGTTCC |
| 174307924 DNA | |
| Sequence | GATCCCTGCCTTCCGTGCCCGCTGGCATTGCTAAACACGTGGAAAGAATCAATTTGGG |
| | GTTTAATAGCATACAGGCCCTGTCAGAAACCTCATTTGCAGGACTGACCAAGTTGGAG |
| | CTACTTATGATTCACGGCAATGAGATCCCAAGCATCCCCGATGGACCTTTAAGAGACC |
| | TCAGCTCTCTTCAGGTTTTCAAGTTCAGCTACAACAAGCTGAGAGTGATCACAGGACA |
| | GACCCTCCAGGGTCTCTCTAACTTAATGAGGCTGCACATTGACCACAACAAGATCAAA |
| | TTTATCCACCCTCAAGCTTTCAACGGCTTAACGTCTCTGAGGCTACTCCATTTGGAAG |
| | GAAATCTCCTCCACCAGCTGCACCCCAGCACCTTCTCCACGTTCACATTTTTGGATTA |
| | TTTCAGACTCTCCACCATAAGGCACCTCTACTTAGCAGAGAACATGGTTAGAACTCTT |
| | CCTGCCAGCATGCTTCCGAACATGCCGCTTCTGGAGAATCTTTACTTGCAGGGAAATC |
| | CGTGGACCTGCGATTGTGAGATGAGATGGTTTTTGGAATGGGATGCAAAGTCCAGAGG |
| | AATTCTGAAGTGTAAAAAGGGCAAAGCTTATGAAGGCGGTCAGTTGTGTGCATGTGC |
| | TTCAGTCCAAAGAAGTTGTACAAACATGAGATTCACAAGCTGAAGGACCTGACTTGTC |
| | TGCTCGAG |

| | |
|---|---|
| ORF Start: CCC at 1 | AORF Stop: it at 763 |

| | | |
|---|---|---|
| SEQ ID NO:6 | 254 aa | MW at 28996.5kD |

| | |
|---|---|
| NOV 1c, | RPCPHPCACYVPSEVHCTFRSLASVPAGIAKHVERINLGFNSIQALSETSFAGLTKLE |
| 174307924 Protein | |
| Sequence | LLMIHGNEIPSIPDGALRDLSSLQVFKFSYNKLRVITGQTLQGLSNLMRLHIDHNKIE |
| | FIHPQAFNGLTSLRLLHLEGNLLHQLHPSTFSTFTFLDYFRLSTIRHLYLAENMVRTL |
| | PASMLRNNPLLENLYLQGNPWTCDCEMRWFLEWDAKSRGILKCKKGAYEGGQLCAMC |
| | FSPKKLYKHEIHKLKDLTCLLE |

| | |
|---|---|
| SEQ ID NO:7 | 762bp |

| | |
|---|---|
| NOV 1d, | CGGCCGTGCCCTCATCCTTGTGCCTGCTACGTCCCCAGCGAGGTCCACTGCACGTTCC |
| 169679197 DNA | |
| Sequence CTACTTATGATTCACGGCAATGAGATCCCAAGCATCCCCGATCGAGCTTTAAGAGACC | |
| | GATCCCTCGCTTCCGTGCCCGCTGGCATTGCTAAACACGTGGAAAGAATCATTTGGG |
| | GTTTAATAGCATACAGGCCCTGTCAGAAACCTCATTTGCAGGACTGACCAAGTTGGAG |
| | TCAGCTCTCTTCAGGTTTTCAAGTTCAGCTACAACAAGCTGAGAGTGATCACAGGACA |
| | GACCCTCCAGGGTCTCTCTAACTTAATGAGGCTGCACATTGACCACAACAAGATCGAG |
| | TTTATCCACCCTCAGCTTTCAACGGCTTAACGTCTCTGAGGCTACTCCATTTGGAG |
| | GAAATCTCCTCCACCAGCTGCACCCCAGCACCTTCTCCACCTTCACATTTTTGGATTA |
| | TTTCAGACTCTCCACCATAAGGCACCTCTACTTAGCAGAGAACATGGTTAGAACTCTT |
| | CCTGCCAGCATGCTTCGGAACATGCCGCTTCTGGAGAATCTTTACTTGCAGGGAAATC |
| | CGTGGACCTGCGATTGTGAGATGAGATGGTTTTTGGAATGGGATGCAAAATCCAGAGG |
| | AATTCTGAAGTGTAAAAAGGACAAAGCTTATGAAGGCGGTCACTTGTGTACATGTGC |
| | TTCAGTCCAAAGAAGTTGTACAACATGAGATTCACAAAGCTGAGGACCTGACTTGTC |
| | TTGCTCGAG |

| | |
|---|---|
| ORF Start: CGG at 1 | ORF Stop: it at 763 |

| | | |
|---|---|---|
| SEQ ID NO: 8 | 254 aa | MW at 29084.6kD |

| | |
|---|---|
| NOV 1d, | RPCPHPCACYVPSEVHCTFRSLASVPAGIAKHVERINLGFNSIQALSETSFAGLTKLE |
| 169679197 Protein | |
| Sequence | LLMIHGNEIPSIPDGALRDLSSLQVFKFSYNKLRVITGQTLQGLSNLMRLHIDHNKIE |
| | FIHPQAFNGLTSLRLLHLEGNLLHQLHPSTFSTFTFLDYFRLSTIRHLYLAENMAARTL |
| | PASMLRNNPLLENLYLQGNPWTCDCEMRWFLEWDAKSRGILKCKKDKAYEGGQLCTMC |
| | FSPKKLYKHEIHKLKDLTCLLE |

| | |
|---|---|
| SEQ ID NO: 9 | 762 bp |

| | |
|---|---|
| NOV 1c, | CGGCCGTGCCCTCATCCTTCTGCCTGCTACGTCCCCAGCGAGGTCCACTGCACGTTCC |
| 169679219 DNA | |
| Sequence | GATCCCTGGCTTCCGTGCCCGCTGGCATTGCTAACACGTGGAAAGAATCAATTTGGG |
| | GTTTAATAGCATACAGGCCCTGTCAGAAACCTCATTTGCAGGACTGACCAAGTTGGAG |
| | CTACTTATGATTCACGGCAATGAGATCCCAAGCATCCCCGATGGAGCTTTAAGAGACC |
| | TCAGCTCTCTTCAGGTTTTCAAGTTCAGCTACAACAAGCTGAGAGTGATCACAGGACA |
| | GACCCTCCAGGGTCTCTCTAACTTAATGAGGCTGCACATTGACCACMA.CIAGATCGAG |
| | TTTATCCACCCTCAAGCTTTCAACGGCTTAACGTCTCTGAGGCTACTCCATTTGGAAG |
| | GAAATCTCCTCCACCAGCTGCACCCCAGCACCTTCTCCACGTTCACATTTTTGGATTA |
| | TTTCAGACTCTCCACCATAAGGCACCTCTACTTAGCAGAGAACATGGTTAGAACTCTT |
| | CCTGCCAGCATGCTTCGGAACATGCCGCTTCTGGAGAATCTTTACTTGCAGGGAAATC |
| | CGTGGACCTGCGATTGTGAGATGAGATGGTTTTTGGAATGGGATGCAAAATCCAGAGG |
| | AATTCTGAAGTGTAAAAAGGACAAAAGCTTATGAAGGCGGTCAGTTGTGTGCPATGTGC |
| | TTCAGTCCAAAGAAGTTGTACAACATGAGATTCACAIGCTGAAGGACCTGACTTGTC |
| | TGCTCGAG |

| | |
|---|---|
| ORE Start: COG at 1 | ORF Stop: it at 763 |

| | | |
|---|---|---|
| SEQ ID NO: 10 | 254aa | MW at 29054.6kD |

| | |
|---|---|
| NOV 1e, | RPCPHPCACYVPSEVHICTFRSLASVPAGIAKIVERINLGFNSIQALSETSFAGLTKLE |
| 169679219 Protein | |
| Sequence | LLMIHCNEIPSIPDGALRDLSSLQVFKFSYNKLRVITGQTLQGLSNLMRLHIDHNKIE |
| | FIHPQAFNGLTSLRLLHLEGNLLHQLHPSTFSTFTFLDYFRLSTIRHLYLAENNVRTL |
| | PASMLRNNPLLENLYLQGNPWTCDCEMRWFLEWDAKSRG ILKCKKDKAYEGGQLCAMC |
| | FSPKKLYKHEIHKLKDLTCLLE |

| | |
|---|---|
| SEQ ID NO: 11 | 762 bp |

| | |
|---|---|
| NOV 1f | GGATCCTGCCCTCATCCTTGTGCCTGCTACGTCCCCAGCGAGGTCCACTGCACGTTCC |
| 207704655 DNA | |
| Sequence | GATCCCTGGCTTCCGTGCCCGCTGGCATTGCTAAACACGTGGAAAGAATCAATTTGGG |
| | GTTTAATAGCATACAGGCCCTGTCAGAAACCTCATTTGCAGGACTGACCAAGTTGGAG |
| | CTACTTATGATTCACGGCATGAGATCCCAAGCATCCCCGATCGAGCTTTAAGAGACC |
| | TCAGCTCTCTTCAGGTTTTCAAGTTCAGCTACAACAAGCTGAGAGTGATCACAGGACA |
| | GACCCTCCAGGGTCTCTCTAACTTAATGAGGCTGCACATTGACCACAACAAGATCGAG |
| | TTTATCCACCCTCAGCTTTCIVAACGGCTTIAACGTCTCTGAGGCTACTCCATTTGGAAG |
| | GAAATCTCCTCCACCAGCTGCACCCCAGCACCTTCTCCACGTTCACATTTTTGGATTA |
| | TTTCAGACTCTCCACCATAAGGCACCTCTACTTAGCAGAGAACATGGTTAGAACTCTT |
| | CCTGCCAGCATGCTTCGGAACATGCCGCTTCTGGAGAATCTTTACTTGCAGGGAAIATC |
| | CGTGGACCTGCCATTGTGAGATGAGATGGTTTTTGGAATGGGATGCAAAATCCAGAGG |
| | AATTCTGAAGTGTAAAAAGGACAAAGCTTATGAAGGCGGTCAGTTGTGTACAATGTGC |
| | TTCAGTCCAAGAAGTTGTACAAACATGAGATTCACAAGCTGAAGGACCTGACTTGTC |
| | TGCTCGAG |

| | |
|---|---|
| ORF Start: OGA at 1 | ORE Stop: it at 763 |

| | | |
|---|---|---|
| SEQ ID NO: 12 | 254 aa | AMW at 28975.4kD |

| | |
|---|---|
| NOV 1f, | GSCPHPCACYTPSEVHCTFRSLASVPAGIAKHVERINLGFNSIQALSETSFAGLTKLE |
| 207704655 Protein | |
| Sequence | LLMIHGNEIPSIPDGALRDLSSLQVFKFSYNKLRVITGQTLQGLSNLMRLHIDHNKIE |
| | FIHPQAFNGLTSLRLLNLEGNLLHQLHPSTFSTFTFLDYFRLSTIRHLYLAENMVRTL |
| | PASMLRNMPLLENLYLQGNPWTCDCEMRWFLEWDAKSRGILKCKKDKAYEGGQLCTMC |
| | FSPKKLYKHEIEHKLKDLTCLLE |

Sequence comparison of the above protein sequences yields the following sequence relationships shown in Table 1B.

TABLE 1B
Comparison of NOV1a against NOV1b through NOV1f.

| Protein | NOV1a Residues/ | Identities/ |
|---|---|---|
| Sequence | Match Residues | Similarities for the Matched Region |
| NOV1b | 27 . . . 277 | 234/251 (93%) |
| | 3 . . . 252 | 234/251 (93%) |
| NOV1c | 27 . . . 277 | 234/251 (93%) |
| | 3 . . . 252 | 234/251 (93%) |
| NOV1d | 27 . . . 277 | 234/251 (93%) |
| | 3 . . . 252 | 234/251 (93%) |
| NOV1e | 27 . . . 277 | 235/251 (93%) |
| | 3 . . . 252 | 235/251 (93%) |
| NOV1f | 26 . . . 277 | 234/252 (92%) |
| | 2 . . . 252 | 235/252 (92%) |

Further analysis of the NOV1a protein yielded the following properties in Table 1C.

TABLE 1C
Protein Sequence Properties NOV1a

| | |
|---|---|
| PSort | 0.4371 probability located in outside; 0.1900 probability |
| analysis: | located in lysosome (lumen); 0.1800 probability located in |
| | nucleus; 0.1000 probability located in endoplasmic reticulum |
| | (membrane) |
| SignalP | Cleavage site between residues 27 and 28 |
| analysis: | |

A search of the NOV1 a protein against the Geneseq database, a proprietary database that contains sequences published in patents and patent publication, yielded several homologous proteins shown in Table 1D.

TABLE 1D
Geneseq Results for NOV1a

| | | NOV1a | Identities/ | |
|---|---|---|---|---|
| | Protein/Organism/ | Residues/ | Similarities for | |
| Geneseq | Length [Patent #, | Match | the Matched | Expect |
| Identifier | Date] | Residues | Region | Value |
| AAM41498 | Human polypeptide SEQ ID NO 6429 - | 2211 . . . 2845 | 605/636 (95%) | 0.0 |
| | Homo sapiens, 666 aa. | 31 . . . 666 | 612/636 (96%) | |
| | [WO200153312-A1, 26 JUL. 2001] | | | |
| AAM39712 | Human polypeptide SEQ ID NO 2857 - | 2258 . . . 2845 | 561/589 (95%) | 0.0 |
| | Homo sapiens, 589 aa. | 1 . . . 589 | 567/589 (96%) | |
| | [WO200153312-A1, 26 JUL. 2001] | | | |
| AAB42539 | Human ORFX ORF2303 polypeptide | 2263 . . . 2845 | 556/584 (95%) | 0.0 |
| | sequence SEQ ID NO: 4606 - Homo | 1 . . . 584 | 562/584 (96%) | |
| | sapiens, 584 aa. [WO200058473-A2, | | | |
| | 05 OCT. 2000] | | | |
| ABB19814 | Protein #1813 encoded by probe for | 841 . . . 1349 | 509/509 (100%) | 0.0 |
| | measuring heart cell gene expression - | 1 . . . 509 | 509/509 (100%) | |
| | Homo sapiens, 509 aa. | | | |
| | [WO200157274-A2, 09 AUG. 2001] | | | |
| AAM67586 | Human bone marrow expressed probe | 841 . . . 1349 | 509/509 (100%) | 0.0 |
| | encoded protein SEQ ID NO: 27892 - | 1 . . . 509 | 509/509 (100%) | |
| | Homo sapiens, 509 aa. | | | |
| | [WO200157276-A2, 09 AUG. 2001] | | | |

In a BLAST search of public sequence databases, the NOV1a protein was found to have homology to the proteins shown in the BLASTP data in Table 1E.

TABLE 1E
Public BLASTP Results for NOV1a

| | | NOV1a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NR99 | ADLICAN - Homo sapiens (Human), | 1 . . . 2845 | 2568/2849 (90%) | 0.0 |
| | 2828 aa. | 1 . . . 2828 | 2641/2849 (92%) | |
| Q9Y3Y8 | HYPOTHETICAL 63.9 KDA | 2263 . . . 2845 | 554/584 (94%) | 0.0 |
| | PROTEIN - Homo sapiens (Human), | 1 . . . 584 | 562/584 (95%) | |
| | 584 aa (fragment). | | | |
| Q96SC3 | FIBULIN-6 - Homo sapiens | 1787 . . . 2844 | 284/1075 (26%) | 8e−81 |
| | (Human), 2673 aa (fragment). | 28 . . . 1022 | 443/1075 (40%) | |
| Q96RW7 | HEMICENTIN - Homo sapiens | 1861 . . . 2844 | 268/991 (27%) | 1e−80 |
| | (Human), 5636 aa. | 3064 . . . 3985 | 416/991 (41%) | |
| Q96DN3 | CDNA FLJ31995 FIS, CLONE | 1854 . . . 2831 | 263/988 (26%) | 4e−73 |
| | NT2RP7009236, WEAKLY | 147 . . . 1066 | 416/988 (41%) | |
| | SIMILAR TO BASEMENT | | | |
| | MEMBRANE-PROTEOGLYCAN | | | |
| | CORE PROTEIN PRECURSOR - | | | |
| | Homo sapiens (Human), | | | |
| | 1252 aa (fragment). | | | |

PFam analysis indicated that the NOV1 a protein contains the domains shown in the Table 1F.

TABLE 1F
Domain Analysis of NOV1a

| | | Identities/ | |
|---|---|---|---|
| | | Similarities | |
| | | for the | |
| | NOV1a | Matched | Expected |
| Pfam Domain | Match Region | Region | Value |
| LRRNT: domain 1 of 1 | 26 . . . 54 | 11/31 (35%) | 0.078 |
| | | 22/31 (71%) | |
| LRR: domain 1 of 6 | 56 . . . 80 | 6/26 (23%) | 2.4e+02 |
| | | 20/26 (77%) | |
| LRR: domain 2 of 6 | 81 . . . 104 | 7/25 (28%) | 0.36 |
| | | 20/25 (80%) | |
| LRR: domain 3 of 6 | 105 . . . 128 | 5/25 (20%) | 4.2 |
| | | 18/25 (72%) | |
| LRR: domain 4 of 6 | 129 . . . 152 | 7/25 (28%) | 0.015 |
| | | 21/25 (84%) | |
| LRR: domain 5 of 6 | 153 . . . 176 | 8/25 (32%) | 0.84 |
| | | 18/25 (72%) | |
| LRR: domain 6 of 6 | 185 . . . 208 | 7/25 (28%) | 3.3 |
| | | 17/25 (68%) | |
| LRRCT: domain 1 of 1 | 218 . . . 277 | 18/64 (28%) | 4.1e−05 |
| | | 40/64 (62%) | |
| ig: domain 1 of 13 | 495 . . . 558 | 15/67 (22%) | 3.7e−06 |
| | | 46/67 (69%) | |
| ig: domain 2 of 13 | 593 . . . 654 | 15/65 (23%) | 1.8e−06 |
| | | 44/65 (68%) | |
| ig: domain 3 of 13 | 1011 . . . 1295 | 5/286 (2%) | 5.9e+04 |
| | | 221/286 | |
| | | (77%) | |
| ig: domain 4 of 13 | 1872 . . . 1936 | 13/68 (19%) | 2.5e−06 |
| | | 45/68 (66%) | |
| ig: domains 5 of 13 | 1971 . . . 2033 | 14/66 (21%) | 1.1e−06 |
| | | 43/66 (65%) | |
| IF3: domain 1 of 1 | 2110 . . . 2122 | | 3.5 |
| | | 11/13 (85%) | |
| ig: domain 6 of 13 | 2073 . . . 2130 | 14/61 (23%) | 0.00026 |
| | | 38/61 (62%) | |
| ig: domain 7 of 13 | 2179 . . . 2241 | 18/66 (27%) | 1.8e−08 |
| | | 44/66 (67%) | |
| ig: domain 8 of 13 | 2276 . . . 2344 | 12/72 (17%) | 0.00089 |
| | | 43/72 (60%) | |
| ig: domain 9 of 13 | 2378 . . . 2437 | 21/63 (33%) | 2.4e−11 |
| | | 45/63 (71%) | |
| ig: domain 10 of 13 | 2476 . . . 2537 | 14/65 (22%) | 0.11 |
| | | 39/65 (60%) | |
| ig: domain 11 of 13 | 2574 . . . 2635 | 14/65 (22%) | 2.1e−06 |
| | | 45/65 (69%) | |
| ig: domain 12 of 13 | 2669 . . . 2730 | 12/65 (18%) | 7.3e−05 |
| | | 44/65 (68%) | |
| ig: domain 13 of 13 | 2765 . . . 2829 | 17/68 (25%) | 8.9e−09 |
| | | 47/68 (69%) | |

### Example 2

The NOV2 clone was analyzed, and the nucleotide and polypeptide sequences are shown in Table 2A.

TABLE 2A
NOV2 Sequence Analysis

| | |
|---|---|
| SEQ ID NO: 13 | 1055 bp |

| | |
|---|---|
| NOV2a, | CCAAGAAACAGAATCAAGGCTCGATGCCTGTCCTGCACGTCCATGGTTCTGAAGGGC |
| | ATCTGGGGACTCTTGATCATCTTGTCAGTATCATCATCTTGGTCTATTATTCTGGTCA |
| | TCTAGCCACTGCTCAAGAAAAGCAATCTCCAATGAAAAAATTCAGGGAATGCAGTCGG |
| | ATTTTTGGTGAAGATGGTCTGACGCTGAAACTCTTTCTTAAAAGAACTGCTCCCTTTT |
| | CTATTCTATGGACTTTGACTAATTACCTTTATTTACTGGCTTTAAAGAAGCTGACGGC |
| | CACGGATGTCTCCGCTCTGTTCTGTTGTAACAAAGCCTTTGTCTTCTTGCTGTCATGG |
| | ATTGTGCTGAAAGACAGGTTCATGGGAGTGAGGATAGTTGCTGCAATAATGGCAATTA |
| | CCGGCATTGTCATGATGGCATATGCAGATAATTTCCACGCTGATTCCATCATAGGAGT |
| | GGCATTTGCGGTGGGCTCAGCCTCTACATCTGCATTATATAAGGTATTGTTTAAAATG |
| | TTTCTTGGAAGTGCCAACTTTGGGGAAGCTGCACACTTTGTCTCCACCTTGGGTTTCT |
| | TCAATTTGATCTTCATCTCCTTCACCCCAGTCATCTTGTATTTCACCAAGGTGGAGCA |
| | CTGGTCCTCTTTTGCTGCTCTGCCATGGGGCTGTCTCTGTGGGATGGCAGGGCTGTGG |
| | CTGGCCTTCAACATCCTGGTGAATGTTGGGGTGGTGCTGACATACCCAATCCTAATCT |
| | CCATTGGGACAGTGCTCAGCGTTCCTGGAAATGCAGCTGTGGATCTCCTAAAGCAGGA |
| | GGTGATATTCAATGTTGTCCGCCTGGCTGCTACCATCATCATCTGCATTGGGTTTCTG |
| | CTGATGCTGTTGCCTGAGGAATGGGATGAAATCACCCTGAGGTTCATCAACAGCCTGA |
| | AGGAAAAGAAGAGTGAGGAGCATGTGGATGATGTGACTGATCCCAGCATACACCTGCG |
| | GGGCAGAGGCAGAGCCAATGGGACAGTGTCTATACCACTGGCTTAGAGAGGGACATAT |
| | TTTGAATGCAC |

| | |
|---|---|
| ORF Start: ATG at 25 | ORF Stop: TAG at 1030 |

| | | |
|---|---|---|
| SEQ ID NO: 14 | 335 aa | MW at 36928.2kD |

| | |
|---|---|
| NOV 2a | MPVLHVHGSEGHLGTLDHLVSIIILVYYSGHLATAQEKQSPMKKFRECSRIFGEDGLT |
| CG58598-01 Protein | |
| Sequence | LKLFLKRTAPFSILWTLTNYLYLLALKKLTATDVSALFCCNKAFVFLLSWIVLKDRFM |
| | GVRIVAAIMAIGIVMMAYADNFHADSIIGVAFAVGSASTSALYKVLFKMFLGSANFG |
| | EAAHFVSTLGFFNLIFISFTPVILYFTKVEHWSSFAALPWGCLCGMAGLWLAFNILVN |
| | VGVVLTYPILISIGTVLSVPGNAAVDLLKQEVIFNVVRLAATIIICIGFLLMLLPEEW |
| | DEITLRFINSLKEKKSEEHVDDVTDPSIHLRGRGRANGTVSIPLA |

| | |
|---|---|
| SEQ ID NO: 15 | 1154 bp |

| | |
|---|---|
| NOV2b, | CCAAGAAAACAGAATCAAGGCTCGCTGCCATGGTTCTGAAGGGC |
| CG58598-02 DNA | |
| | ATCTGGGACCCTTGATCATCTTGTCAGTATCATCATCTTGGGTTGGAACTACACAGA |
| | TTGTAAAAAATTACTTATAAGAACTTCTATTGCCCATTTTTCATGACTTGGTTTTCAAC |
| | AAACTGAACATTATGTTTTTCCCAGTCTATTATTCTGGTCATCTAGCCACTGCTCAA |
| | GAAAAGCAATCTCCAATGAAAAAATTCAGGGAATGCAGTCGGATTTTTGGTGAAGATG |
| | GTCTGACGCTGAAACTTTCTTAAAAGAACTGCTCCCTTTTCTATTCTATGGACTTT |
| | GACTAATTACCTTTATTTACTCCCTTTAAACAACCTGACGGCCACGGATGTCTCCGCT |
| | CTGTTCTGTTGTAACAAGCCTTTGTCTTCTTGCTGTCATGGATTGTCCTGAAAGACA |
| | GGTTCATGGGAGTGAGGATAGTTGCTGCAATAATGGCAATTACCCCCAAGACAAAAA |
| | GGCATATGCAGATAATTTCCACGCTGATTCCATCATAGGACTGGCATTTGCGGTGGGC |
| | TCAGCCTCTACATCTGCATTATATAAGGTCTTGTTTAAAATGTTTCTTGGAGTGCCA |
| | ACTTTGGGGAAGCTGCACACTTTGTCTCCACCTTGGGTTTCTTCAATTTGATCTTCAT |
| | CTCCTTCACCCCAGTCATCTTGTATTTCACCAAGGTGGAGCACTGGTCCTCTTTTGCT |
| | GCTCTGCCATGCGGCTGTCTCTGTGGGATGGCAGGGCTGTGGCTGGCCTTCACATCC |
| | TGGTGAATGTTGGGGTGGTGCTGACATACCCAATCCTAAACTCCATTGGGACAATAAA |
| | CAGCGTTCCTGGAAATGCAGCTGTGGATCTCCTAAAGCAGGAGGTGATATTCAATGTT |
| | GTCCGCCTGGCTGCTACCATCATCATCTGCATTGGGTTTCTGCTGATGCTGTTGCCTG |
| | AGGAATGGGATGAAATCACCCTGAGGTTCATCAACAGCCTGAAGGAAACAAAGTAA |
| | GGAGCATGTGGATGATGTGACTGATCCCAGCATACACCTGCGGGGCAGAGCCAGAGCC |
| | AATGGGACAGTGTCTATACCACTGGCTTAGAGAGGGACATATTTTGAATGCA |

| | |
|---|---|
| ORF Start: ATG at 44 | ORF Stop: TAG at 1130 |

| | | |
|---|---|---|
| SEQ ID NO: 16 | 362 aa | MW at 40382.4kD |

| | |
|---|---|
| NOV2b, | MVLKGIWGPLIILSVSSSWVGTTQIVKITYKNFYCPFFMTWFSTNWNIMFFPVYYSGH |
| CG58598-02 Protein | |
| Sequence | LATAQEKQSPMKKFRECSRIFGEDGLTLKLFLKRTAPFSILWTLTNYLYLLALKKLTA |
| | TDVSALFCCNKAFVFLLSWIVLKDRFMGVRIVAIMAITGIVMMAYADNFAASIIGV |
| | AFAVGSASTSALYKVLFKNFLGSANFGEAAAAFVSTLGFFNLIFISFTPVILYFTKVEH |
| | WSSFAALPWGCLCGMAGLWLAFNILVNVGVVLTYPILISIGTVLSVPGNAAVDLLKQE |
| | VIFNVVRLAATIIICIGFLLMLLPEEWDEITLRFINSLKEKKSEEHVATDDVTDPS IHLR |
| | GRGRANGTVSIPLA |

| | |
|---|---|
| SEQ ID NO: 17 | 324 bp |

| | |
|---|---|
| NOV2c, | GGATCCTGGGTTGGAACTACACAGATTGTMAAAATTACTTATAAGAAIACTTCTATTGCC |
| 209770459 DNA | |
| Sequence | CATTTTTCATGACTTGGTTTTCAACIAAACTGGAACATTATGTTTTTCCCAGTCTATTA |
| | TTCTGGTCATCTAGCCACTGCTCAAGAAAAGCAATCTCCAATGAAAAAATTCAGGGAA |
| | TGCAGTCGGATTTTTGGTGAAGATGGTCTGACGCTGAAACTCTTTCTTAAAAGAACTG |
| | CTCCCTTTTCTATTCTATGGACTTTGACTAATTACCTTATTTACTGGCTTTAAAGAA |
| | GCTGACGGCCACGGATGTCTCCGCTCTGCTCGAG |

| | |
|---|---|
| ORF Start: GGA at 1 | ORF Stop: 32 at 325 |

| | | |
|---|---|---|
| SEQ ID NO: 18 | 108 aa | MW at 12629.7kD |

| | |
|---|---|
| NOV2c, | GSWVGTTQIVKITYKNFYCPFFMTWFSTNWNIMFFPVYYSGHLATAQEKQSPMKKFRE |
| 209770459 Protein | |
| Sequence | CSRIFGEDGLTLKLFLKRTAPFSILWTLTNYLYLLALKKLTATDVSALLE |

Sequence comparison of the above protein sequences yields the following sequence relationships shown in Table 2B.

TABLE 2B
Comparison of NOV2a against NOV2b through NOV2c.

| Protein | NOV2a Residues/ | Identities/ |
|---|---|---|
| Sequence | Match Residues | Similarities for the Matched Region |
| NOV2b | 26 . . . 335 | 291/310 (93%) |
| | 53 . . . 362 | 291/310 (93%) |
| NOV2c | 26 . . . 95 | 51/70 (72%) |
| | 37 . . . 106 | 51/70 (72%) |

Further analysis of the NOV2a protein yielded the following properties shown in Table 2C.

TABLE 2C
Protein Sequence Properties NOV2a

| | |
|---|---|
| PSort | 0.6850 probability located in endoplasmic reticulum |
| analysis: | (membrane); 0.6400 probability located in plasma membrane; |
| | 0.4600 probability located in Golgi body; 0.1000 probability |
| | located in endoplasmic reticulum (lumen) |
| SignalP | Cleavage site between residues 36 and 37 |
| analysis: | |

A search of the NOV2a protein against the Geneseq database, a proprietary database that contains sequences published in patents and patent publication, yielded several homologous proteins shown in Table 2D.

TABLE 2D
Geneseq Results for NOV2a

| | | NOV2a | Identities/ | |
|---|---|---|---|---|
| | Protein/Organism/ | Residues/ | Similarities for | |
| Geneseq | Length [Patent #, | Match | the Matched | Expect |
| Identifier | Date] | Residues | Region | Value |
| AAG28842 | Arabidopsis thaliana protein fragment | 73 . . . 289 | 55/231 (23%) | 4e−09 |
| | SEQ ID NO: 34212 - Arabidopsis | 14 . . . 240 | 104/231 (44%) | |
| | thaliana, 278 aa. [EP1033405-A2, | | | |
| | 06 SEP. 2000] | | | |
| AAG28841 | Arabidopsis thaliana protein fragment | 73 . . . 289 | 55/231 (23%) | 4e−09 |
| | SEQ ID NO: 34211 - Arabidopsis | 94 . . . 320 | 104/231 (44%) | |
| | thaliana, 358 aa. [EP1033405-A2, | | | |
| | 06 SEP. 2000] | | | |
| AAG28840 | Arabidopsis thaliana protein fragment | 73 . . . 289 | 55/231 (23%) | 4e−09 |
| | SEQ ID NO: 34210 - Arabidopsis | 174 . . . 400 | 104/231 (44%) | |
| | thaliana, 438 aa. [EP1033405-A2, | | | |
| | 06 SEP. 2000] | | | |
| AAG38623 | Arabidopsis thaliana protein fragment | 73 . . . 289 | 55/231 (23%) | 6e−09 |
| | SEQ ID NO: 47675 - Arabidopsis | 14 . . . 240 | 103/231 (43%) | |
| | thaliana, 305 aa. [EP1033405-A2, | | | |
| | 06 SEP. 2000] | | | |
| AAG38622 | Arabidopsis thaliana protein fragment | 73 . . . 289 | 55/231 (23%) | 6e−09 |
| | SEQ ID NO: 47674 - Arabidopsis | 94 . . . 320 | 103/231 (43%) | |
| | thaliana, 385 aa. [EP1033405-A2, | | | |
| | 06 SEP. 2000] | | | |

In a BLAST search of public sequence databases, the NOV2a protein was found to have homology to the proteins shown in the BLASTP data in Table 2E.

TABLE 2E
Public BLASTP Results for NOV2a

| | | NOV2a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9JJG8 | BRAIN CDNA, CLONE MNCB- | 1 . . . 335 | 323/335 (96%) | 0.0 |
| | 0335 - Mus musculus (Mouse), 335 | 1 . . . 335 | 330/335 (98%) | |
| | aa. | | | |
| AAL39312 | GH20388P - Drosophila | 26 . . . 301 | 97/278 (34%) | 5e−41 |
| | melanogaster (Fruit fly), 578 aa. | 265 . . . 539 | 157/278 (55%) | |
| Q95XC7 | HYPOTHETICAL 37.3 KDA | 47 . . . 287 | 84/242 (34%) | 4e−34 |
| | PROTEIN - Caenorhabditis elegans, | 87 . . . 326 | 138/242 (56%) | |
| | 339 aa. | | | |
| Q9VDJ2 | CG15688 PROTEIN - Drosophila | 26 . . . 127 | 45/103 (43%) | 7e−13 |
| | melanogaster (Fruit fly), 365 aa. | 265 . . . 365 | 62/103 (59%) | |
| Q9VDJ0 | CG15689 PROTEIN - Drosophila | 143 . . . 298 | 42/169 (24%) | 5e−12 |
| | melanogaster (Fruit fly), 195 aa. | 9 . . . 176 | 82/169 (47%) | |

PFam analysis indicates that the NOV2a protein contains the domains shown in the Table 2F.

TABLE 2F
Domain Analysis of NOV2a

| | NOV2a | Identities/ | |
|---|---|---|---|
| | Match | Similarities | Expect |
| Pfam Domain | Region | for the Matched Region | Value |
| DUF6: domain 1 of 1 | 25 . . . 135 | 18/127 (14%) | 0.89 |
| | | 78/127 (61%) | |

### Example 3

The NOV3 clone was analyzed, and the nucleotide and polypeptide sequences are shown in Table 3A.

TABLE 3A
NOV3 Sequence Analysis

| | |
|---|---|
| SEQ ID NO: 19 | 1681 bp |

| | |
|---|---|
| NOV3a, | AAAGCGCTGACAGCTCAAATGGATCCCATGGAACTGAGAAATGTCAACATCGAACCAG |
| CG57833-01 DNA | |
| Sequence | ATGATGAGAGCAGCAGTGGACAAAGTGCTCCAGATAGCTACATCGGGATAGGAAATTC |
| | AGAAAGGCAGCAATGAGTCAATTTGCTPATGAAGACACTGIAAAGTCAGAATTCCTG |
| | ACAAATGGATTTTTGGGGAAAAAGAAGCTGGCAGATTATGCTGATGAACACCATCCCG |
| | GAACCACTTCCTTTGGAATGTCTTCATTTAACCTGAGTAATGCCATCATCCCCACTCC |
| | GATCCTGGGCTTGTCCTATGCCATGGCCAACACAGGGATCATACTTTTTATGATCATG |
| | CTGCTTGCTGTGGCAATATTATCACTGTATTCAGTTCACCTTTTATTAAAAACAGCCA |
| | AGGAAGGAGGTTCTTTGATTTATGAAAAATTAGGAGAAAAGGCATTTGGATGGCCCGG |
| | AAAAATTGGAGCTTTTGTTTCCATTACAATGCAGAACATTGGAGCAATGTCAAGCTAC |
| | CTCTTTATCATTAAATATGAACTACCTGAAGTAATCAGAGCATTCATGGGACTTGAAG |
| | AAAATACTGGGGAATGGTACCTCAATGGCAACTACCTCATCATATTTGTGTCTGTTCG |
| | AATTATTCTTCCACTTTCGCTCCTTAAAAATTTAGGTTATCTTGGCTATACCAGTGGA |
| | TTTTCTCTTACCTGCATGGTGTTTTTTGTTAGTGTGGTAATTTACAAGATTCCAAAAA |
| | TACCCTGCCCTCTACCTGTTTTGGATCACAGTGTTGGAATCTGTCATTCAACAACAC |
| | GCTTCCAATGCATGTGGTAATGTTACCCAACAACTCTGACAGTTCTGATGTGAACTTC |
| | ATGATGGATTACACCCACCGCAATCCTGCAGGGCTGGATGAGAACCAGGCCAGGGCT |
| | CTCTTCATGACAGTGGAGTAGAATATGAAGCTCATAGTGATGACAAGTGTGAACCCAA |
| | ATACTTTGTATTCAACTCCCGGACGGCCTATGCAATTCCTATCCTAGTATTTGCTTTT |
| | GTATGCCACCCTGAGCTCCTTCCCATCTACAGTGAACTTAAAAGAGCGGTCCCGGAGAA |
| | AAATGCAAACGGTGTCAAAATATTTCCATCACGGGGATGCTTGTCATGTACCTGCTTGC |
| | CGCCCTCTTTGGTTACCTACCTTCTATGGTGAGTTGIAGATGAATTACTTCATGCC |
| | TACAGCAAAGTGTATACATTAGACATCCCCCTTCTCATGGTTCGCCTGGCACTCCTTG |
| | TGGCAGTAACACTAACTCTCCCCATTGTCCTCTTCCCAATTCGTACATCAGTGATCAC |
| | ACTGTTATTTCCCAAACGACCCTTCAGCTGGATACGACATTTCCTGATTGCAGCTGTG |
| | CTTATTGCACTTAATAATGTTCTGGTCATCCTTGTGCCAACTATAAAATACATCTTCG |
| | GATTCATAGGTGCTTCTTCTGCCACTATGCTGATTTTTATTCTTCCAGCAGTTTTTTA |
| | TCTTAAACTTGTCAAGAAAGAAACTTTTAGGTCACCCCAAAAGGTCGGGGCTTTAATT |
| | TTCCTTGTGGTTGGAIATATTCTTCATGATTGGAAGCATGGCACTCATTATAAAAATTGACT |
| | GGATTTATGATCCTCCAAATTCCAACCATCACTAACACAAGGAAAATACTTTCTTT |

| | |
|---|---|
| ORF Start: ATG at 19 | ORF Stop: TAA at 1657 |

| | | |
|---|---|---|
| SEQ ID NO: 20 | 546 aa | MW at 60708.4kD |

| | |
|---|---|
| NOV3a, | MDPMELRNVNIEPDDESSSGESAPDSYIGIGNSEKAAMSQFANEDTESQKFLTNGFLG |
| CG57833-01 Protein | |
| Sequence | KKKLADYADEHHPGTTSFCMSSFNLSNAIMGSGILGLSYAIVIANTGIILFMIMLLAVAI |
| | LSLYSVHLLLKTAKEGGSLIYEKLGEKAFGWPGKIGAFVSITMQNIGAMSSYLFIIKY |
| | ELPEVIRAFMGLEENTGEWYLNGNYLIIFVSVGIILPLSLLKNLGYLGYTSCFSLTCM |
| | VFFVSVVIYKKFQIPCPLPVLDHSVGNLSFNNTLPMHVATMLPNSESSDVNFAAVIDYTH |
| | RNPAGLDENQAKGSLHDSGVEYEAHSDDKCEPKYFVFNSRTAYAIPILVFAFVCHPEV |
| | LPIYSELKERSRRKMQTVSNISITGMLVMYLLALFGYLTFYCEVEDELLHAYSKVYT |
| | LDIPLLMRLAVLVAVTLTVPIVLFPIRTSVITLLFPKRPFSWIRHFLIAAVLIALNN |
| | VLVILVPTIKYIFGFIGASSATMLIFILPAVFYLKLVKKETFRSPQKGALIFLVVGI |
| | FFMIGSMALIIIDWIYDPPNSKHH |

Further analysis of the NOV3a protein yielded the following properties shown in Table 3B.

TABLE 3B
Protein Sequence Properties NOV3a

| | |
|---|---|
| PSort | 0.6000 probability located in plasma membrane; 0.4000 |
| analysis: | probability located in Golgi body; 0.3000 probability |
| | located in endoplasmic reticulum (membrane); 0.0300 |
| | probability located in mitochondrial inner membrane |
| SignalP | No Known Signal Sequence Indicated |
| analysis: | |

A search of the NOV3a protein against the Geneseq database, a proprietary database that contains sequences published in patents and patent publication, yielded several homologous proteins shown in Table 3C.

TABLE 3C
Geneseq Results for NOV3a

| | | NOV3a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAY79188 | Haematopoietic stem cell specific | 1 . . . 546 | 474/547 (86%) | 0.0 |
| | protein - Mus musculus, 547 aa. | 1 . . . 547 | 509/547 (92%) | |
| | [WO200011168-A2, 02-MAR-2000] | | | |
| AAB93237 | Human protein sequence SEQ ID | 108 . . . 545 | 255/438 (58%) | e−140 |
| | NO: 12239 - Homo sapiens, 406 aa. | 5 . . . 406 | 310/438 (70%) | |
| | [EP1074617-A2, 07-FEB-2001] | | | |
| AAB81002 | Rat neuronal glutamine transporter | 5 . . . 546 | 258/542 (47%) | e−134 |
| | (NGT) amino acid sequence - Rattus | 12 . . . 485 | 347/542 (63%) | |
| | norvegicus, 485 aa. [CN1272545-A, | | | |
| | 08-NOV-2000] | | | |
| AAB92592 | Human protein sequence SEQ ID | 1 . . . 243 | 241/244 (98%) | e−133 |
| | NO: 10833 - Homo sapiens, 259 aa. | 1 . . . 244 | 242/244 (98%) | |
| | [EP1074617-A2, 07-FEB-2001] | | | |
| AAM93430 | Human polypeptide, SEQ ID NO: | 5 . . . 546 | 256/542 (47%) | e−133 |
| | 3060 - Homo sapiens, 487 aa. | 12 . . . 487 | 346/542 (63%) | |
| | [EP1130094-A2, 05-SEP-2001] | | | |

In a BLAST search of public sequence databases, the NOV3a protein was found to have homology to the proteins shown in the BLASTP data in Table 3D.

TABLE 3D
Public BLASTP Results for NOV3a

| | | Residues/ | Similarities for | |
|---|---|---|---|---|
| Accession | | Match | the Matched | |
| Number | Protein/Organism/Length | Residues | Portion | Value |
| Q96916 | AMINO ACID TRANSPORTER | 1 . . . 546 | 544/547 (99%) | 0.0 |
| | HNAT3 (AMINO ACID | 1 . . . 547 | 546/547 (99%) | |
| | TRANSPORTER SYSTEM A3) - | | | |
| | Homo sapiens (Human), 547 aa. | | | |
| Q9EQ25 | AMINO ACID TRANSPORT | 1 . . . 546 | 481/547 (87%) | 0.0 |
| | SYSTEM A3 - Rattus norvegicus (Rat), | 1 . . . 547 | 511/547 (92%) | |
| | 547 aa. | | | |
| BAB84091 | SYSTEM A AMINO ACID | 1 . . . 546 | 473/547 (86%) | 0.0 |
| | TRANSPORTER 3 - Mus musculus | 1 . . . 547 | 508/547 (92%) | |
| | (Mouse), 547 aa. | | | |
| Q9HAV3 | AMINO ACID TRANSPORTER | 1 . . . 545 | 313/545 (57%) | e−170 |
| | SYSTEM A (AMINO ACID | 1 . . . 506 | 379/545 (69%) | |
| | TRANSPORTER SYSTEM A2) - | | | |
| | Homo sapiens (Human), 506 aa. | | | |
| Q96QD8 | PUTATIVE 40-9-1 PROTEIN - Homo | 1 . . . 545 | 312/545 (57%) | e−170 |
| | sapiens (Human), 506 aa. | 1 . . . 506 | 379/545 (69%) | |

PFam analysis indicates that the NOV3a protein contains the domains shown in the Table 3E.

TABLE 3E
Domain Analysis of NOV3a

| | NOV3a | Identities/Similarities | |
|---|---|---|---|
| | Match | for | Expect |
| Pfam Domain | Region | the Matched Region | Value |
| sec Y: domain 1 of 1 | 112 . . . 475 | 61/479 (13%) | 7.5 |
| | | 224/479 (47%) | |
| Aa_trans: domain 1 of 1 | 98 . . . 528 | 107/510 (21%) | 2.3e−51 |
| | | 318/510 (62%) | |

### Example 4

The NOV4 clone was analyzed, and the nucleotide and polypeptide sequences are shown in Table 4A.

TABLE 4A
NOV4 Sequence Analysis

| | |
|---|---|
| SEQ ID NO: 21 | 1152 bp |

| | |
|---|---|
| NOV4a, | ATCCTAAATACTACAATGGTGAATGTGGCCAAGAAGATCTCATCAGATGCTACA |
| CG57853-01 DNA | |
| Sequence | CTTTACCATAATCTGGTGACTGATGAAGAAGGAGACATGTGACTATTCACTC |
| | TGGGATTCTGAAGGTAGGCAAGAAAGACTCATTGAAGAAATCAAGAATGTGAAAGTCA |
| | AAGTGCTCAAACAAAAAGACAGTCTACTCCAGGCACCAATGCATATTGATAGACAT |
| | CCTAATGCTTATTTTACCACTAATACTATTGATAAGTGTGCATTTGGTTGTA.AGATT |
| | GAATTACAGCTGTTTCAAZACAGTATGGAAGAGACCTTTGCCAGTIAAATTCTTGGGGCAG |
| | TTACACAGTTTTTTCTGATGCCATTTTGCGGGTTTCTTTTGTCTCAGATTOTCCCATT |
| | GCCTGAGGCGCAAGCTTTTGGAGTTGTAATGACCTGCACGTGCCCAGGAGGGGGTGGG |
| | GGCTATCTCTTTGCTCTGCTTCTAGATGGAGATTTCACATTGGCCATTTTGATGACTT |
| | GCACATCAACATTATTGGCTCTGATCATGATGCCTGTCAATTCTTATATATACAGTAG |
| | GATATTAGGGTTGTCAGGTACATTCCATATTCCTGTTTCTAAATTGTGTCAACACTC |
| | CTTTTCATACTTGTGCCAGTATCAATTGGAATAGTCATCAAGCATAGAATACCTGAAA |
| | AAGCAAGCTTCTTAGAGAGAATAATTAGACCTCTGAGTTTTATTTTAAATGTTCGTAGG |
| | AATTTATTTGACTTTCACAGTGGGATTAGTGTTCTTAAAAACAGATAATCTAGAGGTG |
| | ATTCTGTTGGGTCTCTTAGTTCCTGCTTTGGGTTTGCTGTTTGGGTACTCCTTTGCTA |
| | AAGTTTGTACGCTGCCTCTTCCTGTTTGTAAAACTGTTGCTATTGAAAGTGGGATGTT |
| | AATAGTTTCTTAGCTCTTGCCGTTATTCAGCTGTCTTTTCCACAGTCCAAGGCCAAT |
| | TTAGCTTCTGTGGCTCCTTTTACAGTAGCCATGTGTTCTGGATGTGAAATGTTACTGA |
| | TCATTCTAGTTTACAAGGCTAAGAAAAGATGTATCTTTTTCTTACAAGATAAAAGGAA |
| | AAGAATTTCCTAATCTAACAATTAAAGCATTACTGAATTCCTACTCTGG |

| | |
|---|---|
| ORF Start: ATG at 18 | ORF Stop: TAA at 1119 |

| | | |
|---|---|---|
| SEQ ID NO: 22 | 367 aa | MW at 40588.5kD |

| | |
|---|---|
| NOV4a, | MVNVAKKISSDATNFTINLVTDEEGETNVTIQLWDSEGRQERLIEEIKNVKVKVLKQK |
| CG57853-01 Protein | |
| Sequence | DSLLQAPMHIDRNILMLILPLILLNKCAFGCKIELQLFQTVWKRPLPVILGAVTQFFL |
| | MPFCGFLLSQIVALPEAQAFGVVNTCTCPGGGGGYLFALLLDGDFTLAILMTCTSTLL |
| | ALIMNPVNSYIYSRILGLSGTFHIPVSKIVSTLLFILVPVSIGIVIKHRIPEAFLA |
| | RIIRPLSFILMFVGIYLTFTVGLVFLKTDILEVILLGLLVPALGLLFGYSFAKVCTLP |
| | LPVCKTVAIESGMLNSFLALAVIQLSFPQSKANLASVAFTVANCGCEMLLIILVYK |
| | AKKRCIFFLQDKRKRNFLI |

| | |
|---|---|
| SEQ ID NO:23 | 11355 bp |

| | |
|---|---|
| NOV4b, | TTTCAAATTTCAAAATGATTAGAAAACTTTTTATTGTTCTACTTTTGTTGCTTGTGACTATAGAA |
| CG57853-02 DNA | |
| Sequence | GAAGCAAGGATGTCATCGCTCAGTTTTCTGAATATAGAGAAGACTGATACTATTTT |
| | TCACAAAGACTGAAGAAACCATCCTTGTAAGTTCAAGCTACGAAATAAAACGGCCTAA |
| | GCCAAGAAGATCTCATCAGATGCTACAAACTTTACCATA3AATCTGGTGACTGATGAAC |
| | AAGCAGAACAAATGTGACTATTCAACTCTGGGATTCTGAAGGTAGGCAAGAAAAGACT |
| | CATTGAAGAAATCAAGAATGTGAAAGTCAAAGTGCTCAAACAAAAAACACAGTCTACTC |
| | CAGGCACCAATGCATATTGATAGAACATCCTAATGCTTATTTTACCACTATACTAT |
| | TGAATAAGTGTGCATTTGGTTGTAGATTGATTACAGCTGTTTCAACAGTATGGA |
| | GAGACCTTTGCCAGTAATTCTTGGGGCAGTTACACAGTTTTTTCTGATGCCATTTTGC |
| | GGGTTTCTTTTGTCTCAGATTGTGGCATTGCCTGAGGCGCAAGCTTTTGGAGTTGTAA |
| | TGACCTGCACGTGCCCAGGAGGCGGTGGOGGCTATCTCTTTGCTCTGCTTCTAGATCG |
| | AGATTTCACATTGGCCATTTTGATGACTTGCACATCAACATTATTGGCTCTGATCATG |
| | ATGCCTGTCAATTCTTATATATACAGTAGGATATTAGGGTTGTCACGTACATTCCATA |
| | TTCCTGTTTCTAAAATTGTGTCAACACTCCTTTTCATACTTCTGCCAGTATCATTGG |
| | AATAGTCATCAAGCATAGAATACCTGAGCAAAGCTTCTTAGAGAGAAATTAGA |
| | CCTCTGAGTTTTATTTTAATGTTCGTAGGAATTTATTTGACTTTCACAGTGGGATTAC |
| | TGTTCTTAAAAACAGATAATCTAGAGGTGATTCTGTTCGGTCTCTTAGTTCCTGCTTT |
| | GCGTTTGCTGTTTGGGTACTCCTTTGCTAAAGTTTGTACGCTGCCTCTTCCTGTTTGT |
| | AAAACTGTTGCTATTGAAAGTGGGATGTTAAATAGTTTCTTAGCTCTTGCCGTTATTC |
| | AGCTGTCTTTTCCACAGTCCAACGCCAATTTAGCTTCTGTGGCTCCTTTTACAGTAGC |
| | CATGTGTTCTGGATGTGAAATCTTACTGATCATTCTAGTTTACICGCTAGAAAAGA |
| | TGTATCTTTTTCTTACAAGATAAAGGAAAAGAAATTTCCTAATCTAACAATTAAAGC |
| | ATTACTGAATTCCTACTCTGG |

| | |
|---|---|
| ORF Start: ATG at 8 | ORF Stop: TAA at 1322 |

| | | |
|---|---|---|
| SEQ ID NO: 24 | 438 aa | MW at 48870.2kD |

| | |
|---|---|
| NOV4b, | MIRKLFIVLLLLLVTIEEARMSSLSFLNIEKTEILFFTKTEETILVSSSYENKRPNSS |
| CG57853-02 Protein | |
| Sequence | HLFVKIEDPKILQMVNVAKKISSDATNFTINLVTDEEGETNVTIQLWDSEGRQERLIE |
| | EIKNVKVKVLKQKDSLLQAPMHIDRNILMLILPLILLNKCAFGCKIELQLFQTVWKRP |
| | LPVILGAVTQFFLMPFCGFLLSQIVALPEAQAFGWAMTCTCPGGGGGYLFALLLDGDF |
| | TLAILMTCTSTLLALIMMPVNSYIYSRILGLSGTFHIPVSKIVSTLLFILVPVSIGIV |
| | IKHRIPEKASFLERIIRPLSFILMFVGIYLTFTVGLVFLKTDNLEVILLGLLVPALGL |
| | LFGYSFAKVCTLPLPVCKTVAIESGMLNSFLALAVIQLSFPQSKANLASVAPFTVAMC |
| | SGCEMLLIILVYKAKKRCIFFLQDKRKRNFLI |

| | |
|---|---|
| SEQ ID NO: 25 | 1152 bp |

| | |
|---|---|
| NOV4e, | ATCCTAAAATACTACAAATGGTGAATGTGGCCAAGAAGATCTCATCAGATGCTACAAAA |
| CG57853-03 | |
| Sequence | DNACTTTACCATAAATCTGGTGACTGATGAAGAAGGAGAAACAAIAGTGACTATTCAACTC |
| | TGGGATTCTGAAGGTAGGCAGAGACTCATTGAAGATCAAGAATGTGAAAGTCA |
| | AGTGCTCAAACAAAAAGACAGTCTACTCCAGGCACCAATGCATATTGATAGAAACAT |
| | CCTAATGCTTATTTTACCACTAATACTATTGAATAAGTGTGCATTTGGTTGTAGATT |
| | GAATTACAGCTGTTTCAAACAGTATGGAAGAGACCTTTGCCAGTAATTCTTGGGGCAG |
| | TTACACAGTTTTTTCTGATGCCATTTTGCGGGTTTCTTTTGTCTCAGATTGTGGCATT |
| | GCCTGAGGCGCAAGCTTTTGGAGTTGTAATGACCTGCACGTGCCCAGGAGGGGGTGGG |
| | GGCTATCTCTTTGCTCTGCTTCTAGATGGAGATTTCACATTGGCCATTTTGATGACTT |
| | GCACATCAACATTATTGGCTCTGATCATGATGCCTGTCAATTCTTATATATACAGTAG |
| | GATATTAGGGTTGTCAGGTACATTCCATATTCCTGTTTCTAAAITTGTGTCAACACTC |
| | CTTTTCATACTTGTGCCAGTATCAATTGGAATAGTCATCATGCATAGAATACCTGAAA |
| | AAGCAAGCTTCTTAGAGAGAATAATTAGACCTCTGAGTTTTATTTTAATGTTCGTACG |
| | AATTTATTTGACTTTCACAGTGGGATTAGTGTTCTTAIAAACAGATAATCTAGAGGTG |
| | ATTCTGTTGGGTCTCTTAGTTCCTGCTTTGGGTTTGCTGTTTGGGTACCCTTGCTA |
| | AAGTTTGTACGCTGCCTCTTCCTGTTTGTAAAACTGTTGCTATTGAAAGTGGGATGTT |
| | AATAGTTTCTTAGCTCTTGCCGTTATTCAGCTGTCTTTTCCACAGTCCAAGACCAT |
| | TTAGCTTCTGTGGCTCCTTTTACAGTAGCCATGTGTTCTGGATGTGAAATGTTACTGA |
| | TCATTCTAGTTTACAAGGCTAAGAAAAGATGTATCTTTTTCTTACAAGATAAAAGGAA |
| | AAGAAATTTCCTAATCTACAATTAAAGCATTACTGAATTCCTACTCTGG |

| | |
|---|---|
| ORF Start: ATG at 18 | ORF Stop: TAA at 1119 |

| | | |
|---|---|---|
| SEQ ID NO: 26 | 367 aa | MW at 40588.5kD |

| | |
|---|---|
| NOV4c, | MVNVAKKISSDATNFTINLVTDEEGETNVTIQLWDSEGRQERLIEEIKNVKVKVLKQK |
| CG5 7853-03 Protein | |
| Sequence | DSLLQAPMHIDRNILMLILPLILLNKCAFGCKIELQLFQTVWKRPLPVILGAVTQFFL |
| | MPFCGFLLSQIVALPEAQAFGVVMTCTCPGGGGGYLFALLLDGDFTLAILMTCTSTL |
| | ALIMMPVNSYIYSRILGLSGTFHIPVSKIVSTLLFILVPVSIGIVIKIRIPEKASFLE |
| | RIIRPLSFILMFVGIYLTFTVGLVFLKTDNLEVILLGLLVPALGLLFGYSFAKVCTLP |
| | LPVCKTVAIESGMLNSFLALAVIQLSFPQSKANLASVAPFTVAiAICsGCEMLLIILVYK |
| | AKKRCIFFLQDKRKRNFLI |

Sequence comparison of the above protein sequences yields the following sequence relationships shown in Table 4B.

TABLE 4B
Comparison of NOV4a against NOV4b through NOV4c.

| | NOV4a Residues/ | Identities/Similarities |
|---|---|---|
| Protein Sequence | Match Residues | for the Matched Region |
| NOV4b | 1 . . . 367 | 324/367 (88%) |
| | 72 . . . 438 | 324/367 (88%) |
| NOV4c | 1 . . . 367 | 324/367 (88%) |
| | 1 . . . 367 | 324/367 (88%) |

Further analysis of the NOV4a protein yielded the following properties shown in Table 4C.

TABLE 4C
Protein Sequence Properties NOV4a

| | |
|---|---|
| PSort | 0.6000 probability located in plasma membrane; 0.4318 |
| analysis: | probability located in mitochondrial inner membrane; 0.4000 |
| | probability located in Golgi body; 0.3000 |
| | probability located in endoplasmic reticulum (membrane) |
| SignalP | No Known Signal Sequence Indicated |
| analysis: | |

A search of the NOV4a protein against the Geneseq database, a proprietary database that contains sequences published in patents and patent publication, yielded several homologous proteins shown in Table 4D.

TABLE 4D
Geneseq Results for NOV4a

| | | NOV4a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAR77224 | Hamster ileal/renal bile acid | 72 . . . 353 | 90/284 (31%) | 2e−37 |
| | cotransporter - Cricetulus griseus, 348 | 37 . . . 314 | 155/284 (53%) | |
| | aa. [WO9517905-A1, 06-JUL-1995] | | | |
| AAR77225 | Human ileal/renal bile acid | 72 . . . 353 | 93/285 (32%) | 2e−36 |
| | cotransporter - Homo sapiens, 348 aa. | 37 . . . 314 | 155/285 (53%) | |
| | [WO9517905-A1, 06-JUL-1995] | | | |
| AAG91138 | C glutamicum protein fragment SEQ ID | 71 . . . 309 | 79/249 (31%) | 1e−19 |
| | NO: 4892 - Corynebacterium | 44 . . . 278 | 127/249 (50%) | |
| | glutamicum, 335 aa. [EP1108790-A2, | | | |
| | 20-JUN-2001] | | | |
| AAG42824 | Arabidopsis thaliana protein fragment | 80 . . . 317 | 63/246 (25%) | 5e−19 |
| | SEQ ID NO: 53452 - Arabidopsis | 18 . . . 251 | 114/246 (45%) | |
| | thaliana, 288 aa. [EP1033405-A2, | | | |
| | 06-SEP-2000] | | | |
| AAG42823 | Arabidopsis thaliana protein fragment | 80 . . . 317 | 63/246 (25%) | 5e−19 |
| | SEQ ID NO: 53451 - Arabidopsis | 131 . . . 364 | 114/246 (45%) | |
| | thaliana, 401 aa. [EP1033405-A2, | | | |
| | 06-SEP-2000] | | | |

In a BLAST search of public sequence databases, the NOV4a protein was found to have homology to the proteins shown in the BLASTP data in Table 4E.

TABLE 4E
Public BLASTP Results for NOV4a

| | | NOV4a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P09131 | P3 protein - Homo sapiens (Human), | 15 . . . 345 | 142/335 (42%) | 3e−74 |
| | 477 aa. | 131 . . . 465 | 225/335 (66%) | |
| Q9BSL2 | SIMILAR TO PROTEIN P3 - Homo | 20 . . . 345 | 141/330 (42%) | 4e−73 |
| | sapiens (Human), 448 aa. | 107 . . . 436 | 220/330 (65%) | |
| Q60414 | Ileal sodium/bile acid cotransporter (Ileal | 72 . . . 353 | 90/284 (31%) | 7e−37 |
| | Na(+)/bile acid cotransporter) (Na+ | 37 . . . 314 | 155/284 (53%) | |
| | dependent ileal bile acid transporter) (Ileal | | | |
| | sodium-dependent bile acid transporter) | | | |
| | (ISBT) (Sodium/taurocholate | | | |
| | cotransporting polypeptide, ileal) - | | | |
| | Cricetulus griseus (Chinese hamster), | | | |
| | 348 aa. | | | |
| Q62633 | Ileal sodium/bile acid cotransporter (Ileal | 72 . . . 353 | 93/285 (32%) | 2e−36 |
| | Na(+)/bile acid cotransporter) (Na+ | 37 . . . 314 | 154/285 (53%) | |
| | dependent ileal bile acid transporter) (Ileal | | | |
| | sodium-dependent bile acid transporter) | | | |
| | (ISBT) (Sodium/taurocholate | | | |
| | cotransporting polypeptide, ileal) - Rattus | | | |
| | norvegicus (Rat), 348 aa. | | | |
| CAC39447 | BA11L8.1 (SOLUTE CARRIER FAMILY | 72 . . . 353 | 93/285 (32%) | 8e−36 |
| | 10 (SODIUM/BILE ACID | 37 . . . 314 | 155/285 (53%) | |
| | COTRANSPORTER FAMILY), | | | |
| | MEMBER 2) - Homo sapiens (Human), | | | |
| | 348 aa. | | | |

PFam analysis indicates that the NOV4a protein contains the domains shown in the Table 4F.

TABLE 4F
Domain Analysis of NOV4a

| | NOV4a | Identities/ | |
|---|---|---|---|
| | Match | Similarities | Expect |
| Pfam Domain | Region | for the Matched Region | Value |
| SBF: domain 1 of 1 | 77 . . . 261 | 48/208 (23%) | 1.5e−37 |
| | | 145/208 (70%) | |
| ABC-3: domain 1 of 1 | 126 . . . 323 | 32/283 (11%) | 9.2 |
| | | 125/283 (44%) | |

### Example 5

The NOV5 clone was analyzed, and the nucleotide and polypeptide sequences are shown in Table 5A.

TABLE 5A
NOV5 Sequence Analysis

| | |
|---|---|
| SEQ ID NO: 27 | 2804 bp |

| | |
|---|---|
| NOV5a, | CGCGGCGGTGCGCTGCCCGGCGCCATGCTTCTGCTGGGCATCCTAACCCTGGCTTTCG |
| CG57829-01 DNA | |
| Sequence | CCGGGCGAACCGCTGGAGGCTCTGAGCCAGAGCGGGAGGTAGTCGTTCCCATCCGACT |
| | GGACCCGGACATTAACGGCCGCCGCTACTACTGGCGGGGTCCCGACGACTCCGGGGAT |
| | CAGGGACTCATTTTTCAGATCACAGCATTTCAGGAGGACTTTTACCTACACCTGACGC |
| | CGGATGCTCAGTTCTTGGCTCCCGCCTTCTCCACTGAGCATCTGGGCGTCCCCCTCCA |
| | GGGCTCACCGGGGGCTCTTCAGACCTGCGACGCTGCTTCTATTCTGGGGACGTGAAC |
| | GCCGAGCCGGACTCGTTCGCTGCTCTGAGCCTGTGCGGGGGGCTCCGCGGACCCTTTG |
| | GCTACCGAGGCGCCGAGTATGTCATTAGCCCGCTGCCCAATGCTAGCCCGCCCGCGGC |
| | GCACCGCAACAGCCAGGGCGCACACCTTCTCCAGCGCCGGGGTGTTCCGGGCGGGCCT |
| | TCCGGAGACCCCACCTCTCGCTCCGCGGTGGCCTCGGGCTGGAACCCCGCCATCCTAC |
| | GGGCCCTGGACCCTTACAAGCCGCGGCGGGCGGGCTTCGGGGAGAGTCGTACCCGGCG |
| | CAGGTCTGGGCGCGCCAAGCGTTTCGTGTCTATCCCGCGGTACGTGGAGACGCTGGTG |
| | GTCGCGGACGAGTCAATGGTCAAGTTCCACGGCGCGGACCTGGAACATTATCTGCTGA |
| | CGCTGCTGGCAACGGCGGCGCGACTCTACCGCCATCCCAGCATCCTCAACCCCATCAA |
| | CATCGTTGTGGTCAAGGTGCTGCTTCTTAGAGATCGTGACTCCGGGCCCAAGGTCACC |
| | GGCAATGCGGCCCTGACGCTGCGCAACTTCTGTGCCTGGCAGAAGAAGCTGAACAAAG |
| | TGAGTGACAAGCACCCCGAGTACTGGGACACTGCCATCCTCTTCACCAGGCAGGACCT |
| | GTGTGGACCCACCACCTGTGACACCCTGGGCATGGCTGATGTGGGTACCATGTGTGAC |
| | CCCAAGAGAAGCTGCTCTGTCATTGAGGACGATGGGCTTCCATCAGCCTTCACCACTG |
| | CCCACGAGCTGGGTCACGTGTTCAACATGCCCCATGACAATGTGAAAGTCTGTGAGGA |
| | GGTGTTTGGGAAGCTCCGAGCCAACCACATGATGTCCCCGACCCTCATCCAGATCGAC |
| | CGTGCCAACCCCTGGTCAGCCTGCAGTGCTGCCATCATCACCGACTTCCTGGACAGCG |
| | GGCACGGTGACTGCCTCCTGGACCAACCCAGCAAGCCCATCTCCCTGCCCGAGGATCT |
| | GCCGGCCGCCAGCTACACCCTGAGCCAGCAGTGCGAGCTGGCTTTTGGCGTGGGCTCC |
| | AAGCCCTGTCCTTACATGCACTACTGCACCAAGCTGTGGTGCACCGGGAAGGCCAAGG |
| | GACAGATGGTGTGCCAGACCCGCCACTTCCCCTGGGCCGATCGCACCAGCTGTCGCGA |
| | GGGCAAGCTCTGCCTCAAAGGGGCCTGCGTGGAGAGACACAACCTCAACAAGCACTCT |
| | TCCTCACAGGTGGATGGTTCCTGGGCCAAATGGGATCCCTATGGCCCCTGCTCGCGCA |
| | CATGTGGTCGGGGCGTGCAGCTGGCCAGGAGGCAGTGCACCAACCCCACCCCTGCCAA |
| | CGGGGGCAAGTACTGCGAGGGAGTGAGGGTGAAATACCGATCCTGCAATCTGGAGCCC |
| | TGCCCCAGCTCCGGAAAGAGCTTCCGGGAGGAGCAGTGTGAGGCTTTCAACGGCTACA |
| | ACCACAGCACCAACCGGCTCACTCTCGCCGTGGCATGGGTGCCCAAGTACTCCGGCGT |
| | GTCTCCCCGGGACAAGTGCAAGCTCATCTGCCGAGCCAATGGCACTGGCTACTTCTAT |
| | GTGCTGGCACCCAAGGTGGTGGACGCCACGCTGTGCTCTCCTGACTCCACCTCCGTCT |
| | GTGTCCAAGGCAAGTGCATCAAGGCTGGCTGTGATGGGAACCTGGGCTCCAAGAAGAG |
| | ATTCGACAAGTGTGGGGTGTGTGGGGGAGACAATAAGAGCTGCAAGAAGGTGACTGGA |
| | CTCCTTTCCCCCGCCAGGCATGGCTACAATTTCGTGGTGGCCATCCCCGCACGCGCCT |
| | CAAGCATCGACATCCGCCAGCGCGGTTACAAAGGGCTGATCGGGCATGACAACTACCT |
| | GGCTCTGAAGAACAGCCAAGGCAAGTACCTGCTCAACGGGCATTTCGTGGTGTCGGCG |
| | GTCGAGCGGCACCTGGTGGTGAAGGGCAGTCTGCTGCGGTACAGCGGCACGGGCACAG |
| | CGGTGGAGAGCCTGCAGGCTTCCCCGCCCATCCTCGAGCCGCTGACCGTGGAGGTCCT |
| | CTCCGTGGGGAAGATGACACCGCCCCCGGTCCGCTACTCCTTCTATCTGCCCAAAGAG |
| | CCTCGGGAGGACAAGTCCTCTCATCCCCCGGCACGCTGGGTGGCTGGCAGCTGGGGGC |
| | CGTGCTCCGCGAGCTGCGGCAGTGGCCTGCAGAAGCGGGCGGTGGACTGGCGGCGCTC |
| | CGCCGGGCAGCGCACGGTCCCTGCCTGTGATGCAGCCCATCGGCCCGTGGAGACACAA |
| | GCCTGCGGGGAGCCCTGCCCCACCTGGGAGCTCAGCGCCTGGTCACCCTGCTCCAAGA |
| | GCTGCGGCCGGGGATTTCAGAGGCGCTCACTCAAGTGTGTGGGCCACGGAGGCCCCCT |
| | GCTGGCCCGGGACCAGTGCAACTTGCACCGCAAGCCCCAGGAGCTGGACTTCTCCGTC |
| | CTGAGGCCGTGCTGAGTGGG |

| | |
|---|---|
| ORF Start: ATG at 25 | ORF Stop: TGA at 2797 |

| | | |
|---|---|---|
| SEQ ID NO: 28 | 924aa | MW at 100396.3kD |

| | |
|---|---|
| NOV5a, | MLLLGILTLAFAGRTAGGSEPEREVVVPIRLDPDINGRRYYWRGPEDSGDQGLIFQIT |
| CG57829-01 Protein | |
| Sequence | AFQEDFYLHLTPDAQFLAPAFSTEHLGVPLQGLTGGSSDLRRCFYSGDVNAEPDSFAA |
| | VSLCGGLRGAFGYRGAEYVISPLPNASAPAAQRNSQGAHLLQRRGVPGGPSGDPTSRC |
| | GVASGWNPAILRALDPYKPRRAGFGESRSRRRSGRAKRFVSIPRYVETLVVADESMVK |
| | FHGADLEIIYLLTLLATAARLYRHPSILNPINIVVVKVLLLRDRDSGPKVTGNAALTLR |
| | NFCAWQKKLNKVSDKAIPEYWDTAILFTRQDLCCATTCDTLGMADVGTMCDPKRSCSVI |
| | EDDCLPSAFTTAHELGHVFNMPHDNVKVCEEVFGKLRANHMMSPTLIQIDRANPWSAC |
| | SAAIITDFLDSGHGDCLLDQPSKPISLPEDLPGASYTLSQQCELAFGVGSKPCPYMQY |
| | CTKLWCTGKAKGQMVCQTRHFPWADGTSCGEGKLCLKGACVERHNLNKHSSSQVDGSW |
| | AKWDPYGPCSRTCGGGVQLARRQCTNPTPANGGKYCEGVRVKYRSCNLEPCPSSGKSF |
| | REEQCEAFNGYNHSTNRLTLAVAWVPKYSGVSPRDKCKLICRANGTGYFYVLAPKVVD |
| | GTLCSPDSTSVCVQGKCIKAGCDGNLGSKKRFDKCGVCGGDNKSCKKVTGLLSPARHO |
| | YNFVVAIPAGASS IDIRQRGYKGLIGDDNYLALKNSQGKYLLNGHFVVSAVERDLVVK |
| | GSLLRYSGTGTAVESLQASRPILEPLTVEVLSVGKMTPPRVRYSFYLPKEPREDKSSH |
| | PPARWVACSWCPCSASCGSGLQKRAVDWRGSAGQRTVPACDAAHRPVETQACGEPCPT |
| | WELSAWSPCSKSCGRGFQRRSLKCVGHGGRLLARDQCNLHRKPQELDFCVLRPC |

| | |
|---|---|
| SEQ ID NO: 29 | 2297bp |

| | |
|---|---|
| NOV5b, | CGCGGCGGTGCGCTGCCCGGCGCCATGCTTCTGCTGGGCATCCTAACCCTGGCTTTCG |
| CG57829-05 DNA | |
| Sequence | CCGGGCGAACCGCTGGAGGCTCTGAGCCAGAGCGGGAGGTAGTCGTTCCCATCCGACT |
| | GGACCCGGACATTAACGGCCGCCGCTACTACTGGCGGGGTCCCGAGGACTCCGGGGAT |
| | CAGGGACTCATTTTTCAGATCACAGCATTTCAGGAGGACTTTTACCTACACCTGACGC |
| | CGGATGCTCAGTTCTTGGCTCCCGCCTTCTCCACTGAGCATCTGCGCGTCCCCCTCCA |
| | GGGGCTCACCGGCGGCTCTTCAGACCTGCGACGCTGCTTCTATTCTGGGGACGTGAAC |
| | GCCGAGCCGGACTCGTTCGCTGCTGTGAGCCTGTGCGGGGGGCTCCGCGGAGCCTTTG |
| | GCTACCGAGGCGCCGAGTATGTCATTAGCCCGCTGCCCAATGCTAGCGCGCCGGCGGC |
| | GCAGCGCAACAGCCAGCGCGCACACCTTCTCCAGCGCCGGGGTGTTCCGGGCGGCCCT |
| | TCCGGAGACCCCACCTCTCGCTGCGGGGTGGCCTCGGGCTGGAACCCCGCCATCCTAC |
| | GGGCCCTGGACCCTTACAAGCCGCGGCGGGCGGGCTTCGGGGAGAGTCGTAGCCGGCG |
| | CAGGTCTGGGCGCGCCAAGCGTTTCGTGTCTATCCCGCGGTACGTGGAGACGCTGGTG |
| | GTCGCGGACGAGTCAATGGTCAAGTTCCACGGCGCGGACCTGGAACATTATCTGCTGA |
| | CGCTGCTGGCAACGGCGGCGCGACTCTACCGCCATCCCAGCATCCTCAACCCCATCAA |
| | CATCGTTGTGGTCAAGGTGCTGCTTCTTAGAGATCGTGACTCCGGGCCCAAGGTCACC |
| | GGCAATGCGGCCCTGACGCTGCGCAACTTCTGTGCCTGGCAGAAGAAGCTGAACAAAG |
| | TGAGTGACAAGCACCCCGAGTACTGGGACACTGCCATCCTCTTCACCAGGCAGGTGGA |
| | TGGTTCCTGGGCCAAATGGGATCCCTATGGCCCCTGCTCGCGCACATGTGGTGGGGGC |
| | GTGCAGCTGGCCAGGAGGCAGTGCACCAACCCCACCCCTGCCAACGGGGGCAAGTACT |
| | GCGAGGGAGTGAGGGTGAAATACCGATCCTGCAATCTGGAGCCCTGCCCCAGCTCAGC |
| | CTCCGGAAAGAGCTTCCGGGAGGAGCAGTGTGAGGCTTTCAACGGCTACAACCACAGC |
| | ACCAACCGGCTCACTCTCGCCGTGGCATGGGTGCCCAAGTACTCCGGCGTGTCTCCCC |
| | GGGACAAGTCCAAGCTCATCTGCCCAGCCAATGGCACTGGCTACTTCTATGTGCTGGC |
| | ACCCAAGGTGGTGGACGCCACGCTGTGCTCTCCTGACTCCACCTCCGTCTGTCTCCAA |
| | GGCAAGTGCATCAAGGCTGGCTGTGATGGGAACCTGGGCTCCAAGAAGAGATTCGACA |
| | AGTGTGGGGTGTGTGGGGGAGACAATAAGAGCTGCAAGAAGGTGACTGGACTCTTCAC |
| | CAAGCCCATGCATGGCTACAATTTCGTGGTGGCCATCCCCGCAGGCGCCTCAAGCATC |
| | GACATCCGCCAGCGCGGTTACAAAGGGCTGATCGGGGATGACAACTACCTGGCTCTGA |
| | AGAACAGCCAAGGCAAGTACCTGCTCAACGGGCATTTCCTGGTGTCGGCGGTGGAGCG |
| | GGACCTGGTGGTGAAGGGCAGTCTGCTGCGGTACAGCGGCACGGGCACAGCGGTGGAG |
| | AGCCTCCAGGCTTCCCGGCCCATCCTGGAGCCGCTGACCGTGGAGGTCCTCTCCGTGG |
| | GGAAGATGACACCGCCCCGGGTCCGCTACTCCTTCTATCTGCCCAAAGAGCCTCGGGA |
| | GGACAAGTCCTCTCATCCCAAGGACCCCCGGGGACCCTCTGTCTTGCACAACACCGTC |
| | CTCAGCCTCTCCAACCAGGTGGAGCAGCCGGACGACAGGCCCCCTGCACGCTGGGTGG |
| | CTGGCAGCTGGGGGCCGTGCTCCGCGAGCTGCGGCAGTGGCCTGCAGAAGCGGGCGGT |
| | GGACTGCCGGGGCTCCGCCGGGCAGCGCACGGTCCCTGCCTGTGATCCAGCCCATCCG |
| | CCCGTGGAGACACAAGCCTGCGGGGAGCCCTGCCCCACCTGGGAGCTCAGCGCCTGCT |
| | CACCCTGCTCCAAGAGCTGCGGCCGGGGATTTCAGAGGCGCTCACTCAAGTGTGTGGG |
| | CCACGGAGGCCGGCTGCTGGCCCGGGACCAGTGCAACTTGCACCGCAAGCCCCAGGAG |
| | CTGGACTTCTGCGTCCTCAGCCCGTGCTGAGTGGG |

| | |
|---|---|
| ORF Start: ATG at 25 | ORF Stop: TGA at 2290 |

| | | |
|---|---|---|
| SEQ ID NO: 30 | 755 aa | MW at 82147.6kD |

| | |
|---|---|
| NOV5b, | MLLLGILTLAFAGRTAGGSEPEREVVVPIRLDPDINGRRYYWRGPEDSGDQGLIFQIT |
| CG57829-05 Protein | |
| Sequence | AFQEDFYLHLTPDAQFLAPAFSTEHLGVPLQGLTGGSSDLRRCFYSGDVNAEPDSFAA |
| | VSLCGGLRGAFGYRGAEYVISPLPNASAPAAQRNSQGAHLLQRRGVPGGPSGDPTSRC |
| | GVASGWNPATLRALDPYKPRRAGFGESRSRRRSGRAKRFVSIPRYVETLVVADESMVK |
| | FHGADLEHYLLTLLATAARLYRHPSILNPINIVVVKVLLLRDRDSGPKVTGNAALTLR |
| | NFCAWQKKLNKVSDKHPEYWDTAILFTRQVDGSWAKWDPYGPCSRTCGGGVQLARRQC |
| | TNPTPANGGKYCECVRVKYRSCNLEPCPSSASGKSFREEQCEAFNGYNHSTNRLTLAV |
| | AWVPKYSGVSPRDKCKLICRANGTGYFYVLAPKVVDGTLCSPDSTSVCVQGKCIKAGC |
| | DGNLGSKKRFDKCGVCGGDNKSCKKVTGLFTKPMHGYNFVVAIPAGASSIDIRQRGYK |
| | GLIGDDNYLALKNSQGKYLLNGHFVVSAVERDLVVKGSLLRYSGTGTAVESLQASRPI |
| | LEPLTVEVLSVGKNTPPRVRYSFYLPKEPREDKSSHPKDPRGPSVLHNSVLSLSNQVE |
| | QPDDRPPARWVAGSWGPCSASCGSGLQKRAVDCRGSAGQRTVPACDAAHRPVETQACG |
| | EPCPTWELSAWSPCSKSCCRGFQRRSLKCVGHGGRLLARDQCNLHRKPQELDFCvLRp |
| | C |

| | |
|---|---|
| SEQ ID NO: 31 | 555 bp |

| | |
|---|---|
| NOV5c, | AGATCTCGGTACGTGGAGACGCTGGTGGTCGCGGACGAGTCAATGGTCAGTTCCACG |
| 175070495 DNA | |
| Sequence | GCGCGGACCTGGAACATTATCTGCTGACGCTGCTGGCAACGGCGGCGCGACTCTACCG |
| | CCATCCCAGCATCCTCAACCCCATCAACATCGTTGTGGTCAAGGTGCTGCTTCTTAGA |
| | GATCGTGACTCCGGGCCCAAGGTCACCGGCAATACGGCCCTGACGCrGCGCAACTTCT |
| | GTGCCTGGCAGAAGAAGCTGAACAAAGTGAGTGACAAGCACCCCGAGTACTGGGACAC |
| | TGCCATCCTCTTCACCAGGCAGGACCTGTGTGGAGCCACCACCTGTGACACCCTGGGC |
| | ATGGCTGATGTCGGTACCATGTGTGACCCCAAGAGAAGCTGCTCTGTCATTGAGGACC |
| | ATGGGCTTCCATCAGCCTTCACCACTGCCCACGAGCTGGGCCACGTGTTCPACATGCC |
| | CCATGACAATGTGAAAGTCTGTGAGGAGGTGTTTGGGAAGCTCCGAGCCAACCACATG |
| | ATGTCCCCGACCCTCATCCAGATCGACCTCGAG |

| | |
|---|---|
| ORF Start: AGA at 1 | ORF Stop: ig at 556 |

| | | |
|---|---|---|
| SEQ ID NO: 32 | 185 aa | MW at 20725.7kD |

| | |
|---|---|
| NOV5c, | RSRYVETLVVADESMVKFHGADLEHYLLTLLATAARLYRHPS ELNPINIVVVKVLLLR |
| 175070495 Protein | |
| Sequence | DRDSGPKATTGNTALTLRNFCAWQKKLNKVSDKHPEYWDTAILFTRQDLCGATTCDTLG |
| | MADVGTMCDPKRSCSVIEDDGLPSAFTTA1IELGHVFNMPHDNVKVCEEVFGKLRANHM |
| | MSPTLIQIDLE |

| | |
|---|---|
| SEQ ID NO: 33 | 555 bp |

| | |
|---|---|
| NOV5d, | AGATCTCGGTACGTGGAGACGCTGGTGGTCGCGGACGAGTCAATGGTCAAGTTCCACG |
| 175070504 | |
| Sequence | DNAGCGCGGACCTGGAACATTATCTGCTGACGCTGCCGGCAACGGCGGCGCGACTCTACCG |
| | CCATCCCAGCATCCTCAACCCCATCAACATCGTTGTGGTCAAGGTGCTGCTTCTTAGA |
| | GATCGTGACTCCGGGCCCAACGTCACCGGCAATGCGGCCCTGACGCTGCGCAACTTCT |
| | GTGCCTGGCAGAAGAAGCTGAACAAAGTGAGTCACAAGCACCCCGAGTACTGGGACAC |
| | TGCCATCCTCTTCACCAGGCAGGACCTGTGTGGAGCCACCACCTGTGACACCCTGGGC |
| | ATGGCTGATGTGGGTACCATGTGTGACCCCAAGAGAAGCTGCTCTGTCATTGAGGACG |
| | ATGGGCTTCCATCAGCCTTCACCACTGCCCACGAGCTGGGCCACGTGTTCAACATGCC |
| | CATGACAATGTGAAAGTCTGTGAGGAGGTGTTTGGGAAGCTCCGAGCCAACCACATG |
| | ATGTCCCCGACCCTCATCCAGATCGACCTCGAG |

| | |
|---|---|
| ORF Start: AGA at 1 | ORF Slop: ig at 556 |

| | | |
|---|---|---|
| SEQ ID NO: 34 | 185 aa | MW at 20679.6kD |

| | |
|---|---|
| NOV5d, | RSRYVETLVVADESMVKFHGADLEHYLLTLPATAARLYRHPSILNPINIVVVKVLLLR |
| 175070504 Protein | |
| Sequence | DRDSGPKVTGNAALTLRNFCAWQKKLNKVSDKHPEYWDTAILFTRQDLCGATTCDTLG |
| | MADVGTMCDPKRSCSVIEDDGLPSAFTTAHELGHVFNMPHDNVKVCEEVFGKLRAIIHM |
| | MSPTLIQIDLE |

| | |
|---|---|
| SEQ ID NO:35 | 556 bp |

| | |
|---|---|
| NOV5e, | AGATCTCGGTACGTGGAGACGCTGGTGGTCGCGGACGAGTCAATGGTCAAGTTCCACG |
| 175070512 DNA | |
| Sequence | GCGCGGACCTGGAACATTATCTGCTGACGCTGCTGGCAACGGCGGCGCGACTCTACCG |
| | CCATCCCAGCATCCTCAACCCCATCAACATCGTTGTGGTCAAGGTGCTGCTTCTTACA |
| | GATCGTGACTCCGGGCCCAAGGTCACCGGCAATGCGGCCCTGACGCTGCGCAACTTCT |
| | GTGCCTGGCAGAAGAAGCTGAACAAAGTGAGTGACAAGCACCCCGAGTACTGGGACAC |
| | TGTUCCTGGCAGAAGAAACTGAACAAAGTGAGTGACAAGCACCCCGAGTACTGGGACAC |
| | ATGGCTGATGTGGGTACCATGTGTGACCCCAAGAGAAGCTGCTCTGTCATTGAGGACG |
| | ATGGGCTTCCATCAGCCTTCACCACTGCCCACGAGCTGGGCCACGTGTTCAACATGCC |
| | CCATGACAATGTGAAAGTCTGTGAGGAGGTGTTTGGGAAGCTCCGAGCCAACCACATG |
| | ATGTCCCCGACCCTCATCCAGATCGACCTCGAG |

| | |
|---|---|
| ORF Start: AGA at 1 | ORF Stop: ig at 556 |

| | | |
|---|---|---|
| SEQ ID NO: 36 | 185 aa | MW at 20695.6kD |

| | |
|---|---|
| NOV5e, | RSRYVETLVVADESMVKFHGADLEHYLLTLLATAARLYRHPSILNPINIVVVKVLLLR |
| 175070512 Protein | |
| Sequence | DRDSGPKVTGNAALTLRNFCAWQKKLNKVSDKHPEYWDTAILFTRQDLCGATTCDTLG |
| | MADVGTMCDPKRSCSVIEDDGLPSAFTTAHELGHVFNMPHDNVKVCEEVFGKLRANIM |
| | MSPTLIQIDLE |

| | |
|---|---|
| SEQ ID NO: 37 | 555 bp |

| | |
|---|---|
| NOV5f, | AGATCTCGGTACGTGGAGACGCTGGTGGTCGCGGACCAGTCAATGGTCAAGCTCCACG |
| 175070519 DNA | |
| Sequence | GCGCGGACCTGGAACATTATCTGCTGACGCTGCTGGCAACGGCGGCGCGACTCTACCG |
| | CCATCCCAGCATCCTCAACCCCATCAACATCGTTGTGGTCAAGGTGCTGCTTCTTAGA |
| | CATCGTGACTCCGGGCCCAAGGTCACCGGCAATGCGGCCCTGACGCTGCGCAACTTCT |
| | CTGCCTGGCAGAAAGAAGCTGAACAAAGTGAGTGACAAGCACCCCGAGTACTGGGACAC |
| | TGCCATCCTCTTCACCAGGCAGGACCTGTGTGGAGCCACCACCTGTGACACCCTGGGC |
| | ATGGCTGATGTGGGTACCATGTGTGACCCCAAGAGAAGCTGCTCTGTCATTGAGGACG |
| | ATGGGCTTCCATCAGCCTTCACCACTGCCCACGAGCTGGGCCACGTGTTCAACATGCC |
| | CCATGACAATGTGAAAGTCTGTCACGAGGTGTTTGGGAAGCTCCGAGCCAACCACATG |
| | ATGTCCCCGACCCTCATCCAGATCGACCTCGAG |

| | |
|---|---|
| ORF Start: AGA at 1 | ORF Stop: ig at 556 |

| | | |
|---|---|---|
| SEQ ID NO: 38 | 185aa | MW at 20661.6kD |

| | |
|---|---|
| NOV5f, | RSRYVETLVVADESMVKLHGADLEHYLLTLLATAARLYRHPSILNPINIVVVKVLLLR |
| 175070519 Protein | |
| Sequence | DRDSGPKVTGNAALTLRNFCAWQKKLNKVSDKHPEYWDTAILFTRQDLCGATTCDTLG |
| | MADVGTMCDPKRSCSVIEDDGLPSAFTTAHELGHVFNMPHDNVKVCEEVFGKLRANHM |
| | MSPTLIQIDLE |

Sequence comparison of the above protein sequences yields the following sequence relationships shown in Table 5B.

TABLE 5B
Comparison of NOV5a against NOV5b through NOV5f.

| | NOV5a Residues/ | Identities/Similarities |
|---|---|---|
| Protein Sequence | Match Residues | for the Matched Region |
| NOV5b | 494 . . . 924 | 410/473 (86%) |
| | 283 . . . 755 | 413/473 (86%) |
| NOV5c | 218 . . . 398 | 158/181 (87%) |
| | 3 . . . 183 | 158/181 (87%) |
| NOV5d | 218 . . . 398 | 172/181 (95%) |
| | 3 . . . 183 | 172/181 (95%) |
| NOV5e | 218 . . . 398 | 159/181 (87%) |
| | 3 . . . 183 | 159/181 (87%) |
| NOV5f | 218 . . . 398 | 158/181 (87%) |
| | 3 . . . 183 | 158/181 (87%) |

Further analysis of the NOV5a protein yielded the following properties shown in Table 5C.

TABLE 5C
Protein Sequence Properties NOV5a

| | |
|---|---|
| PSort | 0.5469 probability located in outside; 0.1900 |
| analysis: | probability located in lysosome (lumen); 0.1000 probability |
| | located in endoplasmic reticulum (membrane); 0.1000 |
| | probability located in endoplasmic reticulum (lumen) |
| SignalP | Cleavage site between residues 18 and 19 |
| analysis: | |

A search of the NOV5a protein against the Geneseq database, a proprietary database that contains sequences published in patents and patent publication, yielded several homologous proteins shown in Table 5D.

TABLE 5D
Geneseq Results for NOV5a

| | | NOV5a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAG62299 | Human metalloprotease MDTS6 | 1 . . . 924 | 913/953 (95%) | 0.0 |
| | protein - Homo sapiens, 950 aa. | 1 . . . 950 | 915/953 (95%) | |
| | [WO200134785-A1, 17-MAY-2001] | | | |
| AAB21257 | Rat metalloproteinase ADAMTS-5 - | 89 . . . 581 | 459/497 (92%) | 0.0 |
| | Rattus norvegicus, 505 aa. | 13 . . . 505 | 468/497 (93%) | |
| | [WO200053774-A2, 14-SEP-2000] | | | |
| AAB73549 | Human ADAM-type metalloprotease | 1 . . . 924 | 477/955 (49%) | 0.0 |
| | MDTS4, SEQ ID NO: 4 - Homo | 19 . . . 949 | 631/955 (65%) | |
| | sapiens, 950 aa. [JP2001017183-A, | | | |
| | 23-JAN-2001] | | | |
| AAB50011 | Protein; SEQ ID 125 - Homo sapiens, | 1 . . . 924 | 477/955 (49%) | 0.0 |
| | 968 aa. [WO200071577-A1, | 37 . . . 967 | 631/955 (65%) | |
| | 30-NOV-2000] | | | |
| AAB50002 | Human METH1 - Homo sapiens, 950 | 1 . . . 924 | 477/955 (49%) | 0.0 |
| | aa. [WO200071577-A1, 30-NOV-2000] | 19 . . . 949 | 631/955 (65%) | |

In a BLAST search of public sequence databases, the NOV5a protein was found to have homology to the proteins shown in the BLASTP data in Table 5E.

TABLE 5E
Public BLASTP Results for NOV5a

| | | NOV5a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9UH18 | ADAMTS-1 precursor (EC 3.4.24.-) (A | 1 . . . 924 | 477/955 (49%) | 0.0 |
| | disintegrin and metalloproteinase with | 36 . . . 966 | 631/955 (65%) | |
| | thrombospondin motifs 1) | | | |
| | (ADAM-TS 1) | | | |
| | (ADAM-TS1) (METH-1) - | | | |
| | Homo sapiens (Human), 967 aa. | | | |
| T00017 | gene ADAMTS-1 protein - mouse, | 1 . . . 924 | 478/953 (50%) | 0.0 |
| | 951 aa. | 20 . . . 950 | 636/953 (66%) | |
| P97857 | ADAM-TS 1 precursor (EC 3.4.24.-) (A | 1 . . . 924 | 478/953 (50%) | 0.0 |
| | disintegrin and metalloproteinase with | 37 . . . 967 | 636/953 (66%) | |
| | thrombospondin motifs 1) | | | |
| | (ADAMTS-1) | | | |
| | (ADAM-TS1) - Mus musculus (Mouse), | | | |
| | 968 aa. | | | |
| Q9WUQ1 | ADAMTS-1 precursor (EC 3.4.24.-) (A | 1 . . . 924 | 472/952 (49%) | 0.0 |
| | disintegrin and metalloproteinase with | 37 . . . 966 | 638/952 (66%) | |
| | thrombospondin motifs 1) | | | |
| | (ADAM-TS 1) | | | |
| | (ADAM-TS 1) - Rattus | | | |
| | norvegicus (Rat), 967 aa. | | | |
| Q9UP79 | ADAMTS-8 precursor (EC 3.4.24.-) (A | 1 . . . 869 | 423/902 (46%) | 0.0 |
| | disintegrin and metalloproteinase with | 16 . . . 889 | 569/902 (62%) | |
| | thrombospondin motifs 8) | | | |
| | (ADAM-TS 8) | | | |
| | (ADAM-TS8) (METH-2) (METH-8) — | | | |
| | Homo sapiens (Human), 890 aa. | | | |

PFam analysis indicates that the NOV5a protein contains the domains shown in the Table 5F.

TABLE 5F
Domain Analysis of NOV5a

| | | Identities/ | |
|---|---|---|---|
| | | Similarities | |
| | NOV5a Match | for the Matched | Expect |
| Pfam Domain | Region | Region | Value |
| Pep_M12B_propep: | 67 . . . 181 | 30/120 (25%) | 2.3e−06 |
| domain 1 of 1 | | 69/120 (58%) | |
| Reprolysin: domain 1 of 1 | 218 . . . 427 | 69/226 (31%) | 7.4e−09 |
| | | 135/226 (60%) | |
| tsp_1: domain 1 of 3 | 523 . . . 573 | 19/54 (35%) | 1.5e−07 |
| | | 32/54 (59%) | |
| tsp_1: domain 2 of 3 | 817 . . . 868 | 16/55 (29%) | 0.042 |
| | | 29/55 (53%) | |
| tsp_1: domain 3 of 3 | 871 . . . 924 | 16/61 (26%) | 0.0017 |
| | | 36/61 (59%) | |

### Example 6

The NOV6 clone was analyzed, and the nucleotide and polypeptide sequences are shown in Table 6A.

TABLE 6A
NOV6 Sequence Analysis

| | |
|---|---|
| SEQ ID NO: 39 | j2633 bp |

| | |
|---|---|
| NOV6a, | ATTTTCTAACACATTTCTACAATATAATGCATTGTGGATTACTTCATATTGACCAGGA |
| CG59197-01 DNA | |
| Sequence | TATTGTCAATACAATCATCAAACACTGCTCACCTCAATTTTTTTCACTTGGTTTGCCT |
| | GGTGCCACAATGCTTATTATGGATTTTATTGTAGCAGCTGGTAGAGTGGCTTCTTCAG |
| | CTTTTCTCAATGCACCAAGAGTAGAAGCACAAGTTCTTCTOGGATCTTTGGTTTGCTT |
| | TCCCAACTTATATTGTGAACTGCCTTCTCTTCATCCCAACATTCCTGATGTTGCTGTG |
| | TCTCAGTTTACAGATGTTAAGGAACTTATAATCAAAACTGTATTAAGCTCGGCAAGAG |
| | ATGAGCCCTCTGGTCCTCCACGGTGTGTAGCACTTTGTAGTTTAGGTATTTGGATTTG |
| | TGAAGAACTAGTCCATGAGTCTCATCATCCTCAAATTAAGGAAGCTCTGAATGTGATT |
| | TGTGTTTCCTTAAAGTTTACTAATAAAAACAGTAGCCCACGTAGCTTGTAACATGCTTC |
| | ACATGCTGGTTCATTATGTACCTAGACTTCAGATTTACCAGCCTGATTCTCCCTTCAA |
| | AATTATTCAAATCCTAATAGCTACCATCACCCATCTTTTACCAAGTACAGAGGCTTCA |
| | TCTTATGAAATGGACAAGAGGTTGCTACTATCTTTACTTCTCTGCCTTCTGGACTGGA |
| | TCATGGCCTTACCTCTAAGACACTGCTCCAACCATTTCATGCTACGGGAGCAGAAAG |
| | CGATAAAACAGAAAAATCTGTTCTCAATTGCATTTATAAGGTATTACATGGGTGTGTT |
| | TATGGAGCTCAGTGTTTTAGCAATCCAAGGTATTTTCCCATCAGCCTCTCTGATTTGG |
| | CATCTCTACATTATGATCCTTTTATGCATTTGGAAAGTCTGAAAAGAGCCTGAGCCTCT |
| | GCACTCTCCTGACTCAGAACGATCTTCTAACTCCAGCCAGTAACAGAAGTGATAAACT |
| | CAAATGCAGCATGGATTAATCTCTATAGCAGCCCGCACTGTTATTACACATCTGGTA |
| | ATCACCTGGGCCATTATCCAATGAGCGGTGGTCCTGCTATGCTAACAAGTCAGGTGTG |
| | TGAAAATCACGACAATCATTACAGTGAGTACTCAACTTTCTCCTGAACTCTTTGAG |
| | AGTCCAAATATCCAGTTCTTTGTGTTAAATAATACAACCTTAGTGTCCTGTATCCAGA |
| | TCAGATCAGAAGAGAATATGCCTGGAGGAGGTTTATCTCCTGGCCTTGCATCAGCCAA |
| | TTCAAATGTCAGAATCATAGTACGTGATCTCTCTGGAAAATATTCATGGGATTCTGCT |
| | ATACTCTATGGCCCACCTCCTGTAAGTGGCTTGTCAGAACCTACATCTTTCATGCTTT |
| | CATTGTCTCACCAAGAGAAGCCAGAACAGCCTCCGACATCTAATGAATGCTTAGAAGA |
| | TATAACCGTAAAAGATGCACTTTCTCTCCAGTTTAAAAGATTTAGAGAAACTGTACCA |
| | ACTTGGGATACAATAAGAGATGAAGAAGATGTTCTTGATGAGCTCTTGCAGTATTTGG |
| | GTGTTACTACTCCTGAATGCTTACAGAGAACTGGAATCTCACTTAATATTCCTGCTCC |
| | ACAACCTGTGTGCATTTCTGAAAAACAAGAAAATGATGTTATTAATGCTATCCTTAAG |
| | CAACATACAGAAGAAAAAGAATTTGTTGAGAAGCACTTTAATGACTTAAACATGAAAG |
| | CTGTGGAACAAGATGAACCAATACCTCAAAAACCTCAGTCAGCATTTTATTATTGCAG |
| | ATTGCTTCTTAGTATATTGGGAATGAATTCCTGGGACAAACGGAGGAGCTTTCATCTC |
| | CTGAAGAAAAATGAAAAGCTACTTAGAGAACTTAGGAACTTGGATTCAAGGCAGTGGC |
| | GAGAGACACACAAGATTGCAGTATTTTATGTTGCTGAAGGACAAGAAGACAAACACTC |
| | CATTCTCACCAATACAGGAGGAAGTCAAGCATATGAAGATTTTCTAGCTGGTCTTGGT |
| | TGGGAGGTAAATCTTACAAACCATTGTGGTTTTATGGCACCACTACAAAAAAACAAAA |
| | GCACTGGATTGACCACTCCATATTTTGCTACCTCTACAGTAGAGGTAATATTTCACGT |
| | GTCAACAAGAATGCCTTCTGATTCTGATGATTCTTTGACCAAAAAATTGAGACATTTG |
| | GGAAATGATGAAGTGCACATTGTTTGGTCAGAGCATACTAGAGACTACAGGAGAGGAA |
| | TTATTCCCACAGAATTTGGTGATGTCCTTATTCTAATATATCCAATGAAAAATCACAT |
| | GTTCAGTATTCAGATAATGAAAAAACCAGAGGTACCCTTCTTTGGTCCCCTTTTTGAT |
| | GGTGCTATTGTGAATGCAAAGGTTCTACCCATTATGGTTAGAGCAACAGCTATAAATG |
| | CAAGCCGTGCTCTGAAATCTCTGATTCCATTGTATCAAAACTTGTATGAGGAGAGAGC |
| | ACGATACCTGCAAACAATTGTCCAGCACCACTTAGAACCACAACATTTGAAGATTTT |
| | GCAGCACAGGTTTTTTCTCCAGCTCCCTACCACCATTTACCATCTGATGCCGCTAAGA |
| | TTAAAAGCGAGTATTAGTTACTT |

| | |
|---|---|
| ORF Start: ATG at 27 | ORF Stop: TAG at 2625 |

| | | |
|---|---|---|
| SEQ ID NO: 40 | 1866 aa | MW at 97197.4kD |

| | |
|---|---|
| NOV6a, | MHCGLLHIDQDIVNTIIKHCSPQFFSLGLPGATMLIMDFIVAAGRVASSAFLNAPRVE |
| CG59197-01 Protein | |
| Sequence | AQVLLGSLVCFPNLYCELPSLHPNIPDVAVSQFTDVKELIIKTVLSSARDEPSGPARC |
| | VALCSLGIWICEELVHESHHPQIKEALNVICVSLKFTNKTVAHVACNMLHMLVHYVPR |
| | LQIYQPDSPLKIIQILIATITHLLPSTEASSYEMDKRLVVSLLLCLLDWIMALPLKTL |
| | LQPFHATGAESDKTEKSVLNCIYKVLHGCVYGAQCFSNPRYFPMSLSDLASVDYDPFM |
| | HLESLKEPEPLHSPDSERSSKLQPVTEVKTQMQHGLISIAARTVITHLVNHLGHYPMS |
| | GGPAMLTSQVCENHDNHYSESTELSPELFESPNIQFFVLNNTTLVSCIQIRSEENMPG |
| | GGLSAGLASANSNVRIIVRDLSCKYSWDSAILYGPPPVSGLSEPTSFMLSLSHQEKPE |
| | EPPTSNECLEDITVKDGLSLQFKRFRETVPTWDTIRDEEDVLDELLQYLGVTSPECLQ |
| | RTGISLNIPAPQPVCISEKQENDVINAILKQHTEEKEFVEKHFNDLNMKAVEQDEPIP |
| | QKPQSAFYYCRLLLSILGMNSWDKRRSPHLLKKNEKLLRELRNLDSRQWRETHKIAVF |
| | YVAEGQEDKRSILTNTGGSQAYEDFVAGLGWEVNLTNHCGFMGGLQKNKSTGLTTPYF |
| | ATSTVEVIFHVSTRMPSDSDDSLTKKLRHLGADEVHIVWSEHTRDYRRGIIPTEFGDV |
| | LIVIYPMKNHMFSIQIMKKPEVPFFGPLFDGAIVNGKVLPIMVRATAINASRALKSLI |
| | PLYQNLYEERARYLQTIVQHHLEPTTFEDFAAQVFSPAPYHHLPSDAGKIKSEY |

| | |
|---|---|
| SEQ ID NO: 41 | 1923 bp |

| | |
|---|---|
| NOV6b, | GGATCCAAGACACTGCTCCAACCATTTCATGCTACGGGAGCAGAAAGCGATAAACAG |
| 188822075 DNA | |
| Sequence | AAAAATCTGTTCTCAATTGCATTTATAAGGTTTTACATGGGTGTGTTTATGGAGCTCA |
| | GTGTTTTAGCAATCCAAGGTATTTTCCCATGAGCCTCTCTGATTTGGCATCTGTAGAT |
| | TATGATCCTTTTATGCATTTGGAAAGTCTGAAAGAGCCTGAGCCTCTGCACTCTCCTG |
| | ACTCAGAACGATCTTCTAAACTCCAGCCAGTAACAGAAGTGAAAACTCAAATGCAGCA |
| | TGGATTAATCTCTATAGCAGCCCGCACTGTTATTACACATCTGGTAAATCACCTGGGC |
| | CATTATCCAATGAGCGGTGGTCCTGCTATGCTAACAAGTCAGGTGTGTGAAAATCACG |
| | ACAATCATTACAGTGAAAGTACTGAACTTTCTCCTGAACTCTTTGAGAGTCCAAATAT |
| | CCAGTTCTTTGTGTTAAATAATACAACCTTAGTGTCCTGTATCCAGATCAGATCAGA |
| | GAGAATATGCCTGGAGGAGGTTTATCTGCTGGCCTTGCATCAGCCAATTCAAATGTCA |
| | GAATCATAGTACGTGATCTCTCTGGAAAATATTCATGGGATTCTGCTATACTGTATGG |
| | CCCACCTCCTGTAAGTGGCTTGTCACAACCTACATCTTTCATGCTTTCATTGTCTCAC |
| | CAAGAGAAGCCAGAAGAGCCTCCGACATCTAATGAATGCTTAGAAGATATACCGTAA |
| | AAGATGGACTTTCTCTCCAGTTTAAAAGATTTAGAGAAACTGTACCAACTTGGGATAC |
| | AATAAGAGATGAAGAAGATGTTCTTGATGAGCTCTTGCAGTATTTGGGTGTTACTAGT |
| | CCTGATGCTTACAGAGAACTGGAATCTCACTTAATATTCCTGCTCCACAACCTGTGT |
| | GCATTTCTGAAAAACAAGAAAATGATGTTATTAATGCTATCCTTAAGCAACATACAGA |
| | AGAAAAGAATTTGTTGAGAAGCACTTTAATGACTTAACATGAAAGCTGTCCAACAA |
| | GATGAACCAATACCTCAAAAACCTCAGTCAGCATTTTATTATTGCAGATTGCTTCTTA |
| | GTATATTGGGAATGAATTCCTGGGACAAACGGAGGAGCTTTCATCTCCTGAAGAAAAAA |
| | TGAAAAGCTACTTAGAGAACTTAGGAACTTGGATTCAAGGCAGTGCCGAGAGACACAC |
| | AGATTGCAGTATTTTATGTTGCTGAGGACAACAAGACAAACACTCCATTCTCACCA |
| | ATACAGGAGGAAGTCAAGCATATGAAGATTTTGTAGCTGCTCTTCGTTGGGACCTA |
| | TCTTACAAACCATTGTGGTTTTATGGGAGGACTACAAAAAAACAAAAGCACTGGATTG |
| | ACCACTCCATATTTTGCTACCTCTACAGTAGAGGTAATATTTCACGTGTCAACAGAA |
| | TGCCTTCTGATTCTGATGATTCTTTGACCAAAAAATTGAGACATTTGGGAAATGATGA |
| | AGTGCACATTGTTTGGTCAGAGCATACTAGAGACTACAGGAGAGGAATTATTCCCACA |
| | GAATTTGGTGATGTCCTTATTGTAATATATCCAATGAAAAATCACATGTTCAGTATTC |
| | AGATAATGAIAAAAACCAGAGGTTCCCTTCTTTGGTCCCCTTTTTGATGGTGCTATTT |
| | GAATGGAAGGTTCTACCCATTATGGTTAGAGCAACAGCTATAAATGCAAGCCGTGCT |
| | CTGAAATCTCTGATTCCATTCATATCAAACTTCTATGAGGAGAGAGCCGATACCTGC |
| | AAACAATTGTCCAGCACCACTTAGAACCAACAACATTTGAAGATTTTGCAGCACAGGT |
| | TTTTTCTCCAGCTCCCTACCACCATTTACCATCTGATGCCGGTAAGATTAAAAGCGAG |
| | TATCTCGAG |

| | |
|---|---|
| ORF Start: OGA at 1 | ORF Stop: 47 at 1924 |

| | | |
|---|---|---|
| SEQ ID NO: 42 | 641 aa | MW at 72273.2kD |

| | |
|---|---|
| NOV6b, | GSKTLLQPFHATGAESDKTEKSVLNCIYKVLHGCVYGAQCFSNPRYFPMSLSDLASVD |
| 188822075 Protein | |
| Sequence | YDPFMHLESLKEPEPLHSPDSERSSKLQPVTEVKTQMQHGLISIARTVITHLAAAHLG |
| | HYPMSGGPANLTSQCENDNHYSESTELSPELFESPNIQFFVLNNTTLVSCIQIRSE |
| | ENMPGGGLSAGLASANSNVRHVRDLSGKYSWDSAILYGPPPVSGLSEPTSFMLSLSH |
| | QEKPEEPPTSNECLEDITVKDGLSLQFKRFRETVPTWDTIRDEEDVLDELLQYLGVTS |
| | PECLQRTGISLNIPAPQPVCISEKQENDVINAILKQHTEEKEFVEKHFNDLNNKAVEQ |
| | DEPIPQKPQSAFYYCRLLLSILGMNSWDKRRSFHLLKKNEKLLRELRNLDSRQCRETH |
| | KIAVFYVAEGQEDKHSILTNTGGSQAYEDFVAGLGWEVNLTNHCGFMGOLQKNKSTGL |
| | TTPYFATSTVEVIFHVSTRMPSDSDDSLTKKLRHLGNDEVHIVWSEHTRDYRRGIIPT |
| | EFGDVLIVIYPMKNHMFSIQIMKKPEVPFPGPLFDGAIVNCKVLPIMVAATAINASRA |
| | LKSLIPLYQNFYEERARYLQTIVQHHLEPTTFEDFAAQVFSPAPYHHLPSDAGKIKSE |
| | YLE |

Sequence comparison of the above protein sequences yields the following sequence relationships shown in Table 6B.

TABLE 6B
Comparison of NOV6a against NOV6b and NOV6c.

| | NOV6a Residues/ | Identities/Similarities |
|---|---|---|
| Protein Sequence | Match Residues | for the Matched Region |
| NOV6b | 230 . . . 866 | 606/637 (95%) |
| | 3 . . . 639 | 606/637 (95%) |

Further analysis of the NOV6a protein yielded the following properties shown in Table 6C.

TABLE 6C
Protein Sequence Properties NOV6a

| | |
|---|---|
| PSort | 0.7900 probability located in plasma membrane; 0.3000 |
| analysis: | probability located in microbody (peroxisome); 0.3000 |
| | probability located in Golgi body; 0.2000 |
| | probability located in endoplasmic reticulum (membrane) |
| SignalP | No Known Signal Sequence Indicated |
| analysis: | |

A search of the NOV6protein against the Geneseq database, a proprietary database that contains sequences published in patents and patent publication, yielded several homologous proteins shown in Table 6D.

TABLE 6D
Geneseq Results for NOV6a

| | | NOV6a | Identities/ | |
|---|---|---|---|---|
| | | Residues/ | Similarities for | |
| Geneseq | Protein/Organism/Length | Match | the Matched | Expect |
| Identifier | [Patent#, Date] | Residues | Region | Value |
| AAB41763 | Human ORFX ORF1527 polypeptide | 445 . . . 859 | 410/415 (98%) | 0.0 |
| | sequence SEQ ID NO: 3054 - Homo | 1 . . . 415 | 410/415 (98%) | |
| | sapiens, 417 aa. [WO200058473-A2, | | | |
| | 05-OCT-2000] | | | |
| AAB93704 | Human protein sequence SEQ ID | 177 . . . 864 | 389/693 (56%) | 0.0 |
| | NO: 13287 - Homo sapiens, 704 aa. | 1 . . . 658 | 491/693 (70%) | |
| | [EP 1074617-A2, 07-FEB-2001] | | | |
| AAB95195 | Human protein sequence SEQ ID | 599 . . . 819 | 186/221 (84%) | e−107 |
| | NO: 17282 - Homo sapiens, 227 aa. | 1 . . . 221 | 203/221 (91%) | |
| | [EP1074617-A2, 07-FEB-2001] | | | |
| AAR77223 | Tuberous sclerosis 2 TSC2 gene | 610 . . . 857 | 70/260 (26%) | 3e−20 |
| | product - Homo sapiens, 1784 aa. | 1497 . . . 1755 | 128/260 (48%) | |
| | [WO9518226-A, 06-JUL-1995] | | | |
| AAW95629 | Homo sapiens secreted protein gene | 616 . . . 781 | 50/172 (29%) | 1e−14 |
| | clone gm196_4 - Homo sapiens, | 17 . . . 188 | 89/172 (51%) | |
| | 322 aa. [WO9856805-A1, | | | |
| | 17-DEC-1998] | | | |

In a BLAST search of public sequence databases, the NOV6a protein was found to have homology to the proteins shown in the BLASTP data in Table 6E.

TABLE 6E
Public BLASTP Results for NOV6a

| Protein | | NOV6a | Identities/ | |
|---|---|---|---|---|
| Accession | | Residues/ | Similarities for | Expect |
| Number | Protein/Organism/Length | Residues | Portion | Value |
| O55008 | TULIP 2 - Rattus norvegicus (Rat), | 1 . . . 865 | 815/865 (94%) | 0.0 |
| | 866 aa. | 1 . . . 865 | 843/865 (97%) | |
| O55007 | TULIP 1 - Rattus norvegicus (Rat), | 1 . . . 723 | 680/723 (94%) | 0.0 |
| | 747 aa. | 1 . . . 723 | 705/723 (97%) | |
| Q9BQT6 | BA287B20.1.1 (KIAA1272 SIMILAR | 11 . . . 855 | 483/852 (56%) | 0.0 |
| | TO RAT TULIP PROTEINS 1 AND 2, | 1 . . . 817 | 613/852 (71%) | |
| | ISOFORM 1) - Homo sapiens (Human), | | | |
| | 820 aa (fragment). | | | |
| Q9ULE8 | KIAA1272 PROTEIN - Homo sapiens | 1 . . . 817 | 464/824 (56%) | 0.0 |
| | (Human), 1023 aa (fragment). | 231 . . . 1019 | 590/824 (71%) | |
| Q9JMC4 | TULIP 1 PROTEIN - Mus musculus | 131 . . . 570 | 407/440 (92%) | 0.0 |
| | (Mouse), 446 aa. | 7 . . . 446 | 423/440 (95%) | |

PFam analysis indicates that the NOV6a protein contains the domains shown in the Table 6F.

TABLE 6F
Domain Analysis of NOV6a

| | NOV6a | Identities/ | |
|---|---|---|---|
| | Match | Similarities for | Expect |
| Pfam Domain | Region | the Matched Region | Value |
| Rap_GAP: domain 1 of 1 | 650 . . . 829 | 52/192 (27%) | 2.5e−18 |
| | | 98/192 (51%) | |

### Example 7

The NOV7 clone was analyzed, and the nucleotide and polypeptide sequences are shown in Table 7A.

TABLE 7A
NOV7 Sequence Analysis

| | |
|---|---|
| SEQ ID NO: 43 | 570 bp |

| | |
|---|---|
| NOV7a, | CTCATCCCTTTGCGACGTCAATGCGACCACGGGCACCAGGCTTCTCGGCTGGGTAGCT |
| CG58524-11 DNA | |
| Sequence | CTTTGTCTCCTGCCAGCAGGTGAGCTCTGAGCACAGAATAAAAACCCTTGTCCTGGGC |
| | TGGCCAACCCCCAGATCCAAGCTTTGCTGTTGTTGTTIAAGGTCTTTCCTGGACTCTGT |
| | CTACAGCATCTGTTTCTTTCTCTTACAGGCTGTTTTCATGCCAAAGTCACACAGACTC |
| | CAGGATATTTGGTCAAAGGAAAAGCAAAGGAACAAAGATGTATTGTACCCCCAApAAA |
| | CGGACATACTTTTGTTTGTTGGTATCAGCAGAATCAGAATAAAGAGTTTATGTTTTTG |
| | ATTTCCTTTCAGAATGAACAAGTTCTTCAAGAAATCGAGATGCACAAGAGCGATTCT |
| | CATCTCAATGCCCCAAGAACCCACCCTGCAGCCTGGCAAATCCTGTCCTCGGAACCGGG |
| | AGACACCGCACTGTATCTCTGTGCCACCAGTCCGTCCACAGCACTGAAAATGTCAGTTC |
| | CTGTTAGCACACAAACTTGCCACAGACCCAGCTCAGGAGCAGGTGAT |

| | |
|---|---|
| ORF Start: AAA at 100 | ORF Stop: DF at 571 |

| | | |
|---|---|---|
| SEQ ID NO: 44 | 157aa | MW at 17470.1kD |

| | |
|---|---|
| NOV7a, | KPLSWAGQPPDPSFAVVVKVFPGLCLQHLFLSLTGCFHAKVTQTPGYLVKGKGRKTKM |
| CG58524-01 Protein | |
| Sequence | YCTPKNGHTFVCWYQQNQNKEFMFLISFQNEQVLQEMEMHKKRFSSQCPKIAPPCSLAI |
| | LSSEPGDTALYLCATSPSTALKCQFLLAHKLATDPAQEAGD |

Further analysis of the NOV7a protein yielded the following properties shown in Table 7B.

TABLE 7B
Protein Sequence Properties NOV7a

| | |
|---|---|
| PSort | 0.5500 probability located in endoplasmic reticulum |
| analysis: | (membrane); 0.1900 probability located in lysosome (lumen); |
| | 0.1421 probability located in microbody (peroxisome); |
| | 0.1000 probability located in endoplasmic reticulum (lumen) |
| SignalP | Cleavage site between residues 40 and 41 |
| analysis: | |

A search of the NOV7a protein against the Geneseq database, a proprietary database that contains sequences published in patents and patent publication, yielded several homologous proteins shown in Table 7C.

TABLE 7C
Geneseq Results for NOV7a

| | | NOV7a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| ABB25859 | Protein #7858 encoded by probe for | 36 . . . 155 | 120/120 (100%) | 5e−69 |
| | measuring heart cell gene expression - | 1 . . . 120 | 120/120 (100%) | |
| | Homo sapiens, 120 aa. | | | |
| | [WO200157274-A2, 09-AUG-2001] | | | |
| AAM76093 | Human bone marrow expressed probe | 36 . . . 155 | 120/120 (100%) | 5e−69 |
| | encoded protein SEQ ID NO: 36399 - | 1 . . . 120 | 120/120 (100%) | |
| | Homo sapiens, 120 aa. | | | |
| | [WO200157276-A2, 09-AUG-2001] | | | |
| AAM63281 | Human brain expressed single exon | 36 . . . 155 | 120/120 (100%) | 5e−69 |
| | probe encoded protein SEQ ID NO: | 1 . . . 120 | 120/120 (100%) | |
| | 35386 - Homo sapiens, 120 aa. | | | |
| | [WO200157275-A2, 09-AUG-2001] | | | |
| AAM36201 | Peptide #10238 encoded by probe for | 36 . . . 155 | 120/120 (100%) | 5e−69 |
| | measuring placental gene expression - | 1 . . . 120 | 120/120 (100%) | |
| | Homo sapiens, 120 aa. | | | |
| | [WO200157272-A2, 09-AUG-2001] | | | |
| AAY80643 | Canine TCR V-beta 54 protein, SEQ ID | 24 . . . 135 | 60/112 (53%) | 1e−27 |
| | [WO200006732-A2, 10-FEB-2000] | 6 . . . 117 | 75/112 (66%) | |

In a BLAST search of public sequence databases, the NOV7a protein was found to have homology to the proteins shown in the BLASTP data in Table 7D.

TABLE 7D
Public BLASTP Results for NOV7a

| | | NOV7a | Identities/ | |
|---|---|---|---|---|
| Protein | | Residues/ | Similarities for | |
| Accession | | Match | the Matched | Expect |
| Number | Protein/Organism/Length | Residues | Portion | Value |
| C32578 | T-cell receptor beta chain precursor V | 14 . . . 132 | 102/119 (85%) | 2e−55 |
| | region (HBVT72) - human, 120 aa. | 2 . . . 120 | 108/119 (90%) | |
| AAD15201 | T-CELL RECEPTOR BETA | 14 . . . 132 | 102/119 (85%) | 2e−55 |
| | PRECURSOR - Homo sapiens | 2 . . . 120 | 108/119 (90%) | |
| | (Human), 143 aa (fragment). | | | |
| CAB99345 | BA255A11.11 (PUTATIVE NOVEL T | 36 . . . 131 | 96/96 (100%) | 3e−54 |
| | CELL RECEPTOR BETA CHAIN V | 1 . . . 96 | 96/96 (100%) | |
| | REGION PROTEIN) - Homo sapiens | | | |
| | (Human), 96 aa (fragment). | | | |
| AAA36721 | T-CELL RECEPTOR BETA CHAIN - | 26 . . . 114 | 88/89 (98%) | 5e−49 |
| | Homo sapiens (Human), 89 aa | 1 . . . 89 | 88/89 (98%) | |
| | (fragment). | | | |
| CAB35951 | TCRBV19S1P PROTEIN - Homo | 29 . . . 134 | 89/106 (83%) | 1e−46 |
| | sapiens (Human), 116 aa (fragment). | 11 . . . 116 | 93/106 (86%) | |

PFam analysis indicates that the NOV7a protein contains the domains shown in the Table 7E.

TABLE 7E
Domain Analysis of NOV7a

| | | Identities/ | |
|---|---|---|---|
| | | Similarities | |
| | NOV7a | for the | |
| Pfam Domain | Match Region | Matched Region | Expect Value |
| ig: domain 1 of 1 | 53 . . . 131 | 12/83 (14%) | 0.00096 |
| | | 53/83 (64%) | |

### Example 8

The NOV8 clone was analyzed, and the nucleotide and polypeptide sequences are shown in Table 8A.

TABLE 8A
NOV8 Sequence Analysis

| | |
|---|---|
| SEQ ID NO:45 | 1156 bp |

| | |
|---|---|
| NOV8a, | CTAGCGCTCGTACTCCTGGGCTGGGTCTCCTCGTCTTCTCCCACCTCCTCGGCATCCT |
| CG56512-02 DNA | |
| Sequence | CCTTCTCCTCCTCGGCGCCGTTCCTGGCTTCCGCCGTGTCCGCCCAGCCCCCGCTGCC |
| | GGACCAGTGCCCCGCGCTGTGCGAGTGCTCCGAGGCAGCGCGCACAGTCAAGTGCGTT |
| | AACCGCAATCTGACCGAGGTGCCCACGGACCTGCCCGCCTACGTGCGCAACCTCTTCC |
| | TTACCGGCAACCAGCTGGCCGCGCTCAACCTCAGCGGCAGCCGCCTGGACGAGGTGCG |
| | CGCGGGCGCCTTCGAGCATCTGCCCAGCCTGCGCCAGCTCGACCTCAGCCACAACCCA |
| | CTGGCCGACCTCAGTCCCTTCGCTTTCTCGGGCAGCAATGCCAGCGTCTCGGCCCCCA |
| | GTCCCCTTGTGGAACTGATCCTGAACCACATCGTGCCCCCTGAAGATGAGCGGCAGA |
| | CCGGAGCTTCGAGGGCATGGTGGTGGCGGCCCTGCTGGCGGGCCGTGCACTGCAGGGG |
| | CTCCGCCGCTTGGAGCTGGCCAGCAACCACTTCCTTTACCTGCCGCGGGATGTGCTGG |
| | CCCAACTGCCCAGCCTCAGGCACCTGGACTTAAGTAATAATTCGCTGGTGAGCCTGAC |
| | CTACGTGTCCTTCCCCAACCTGACACATCTAGAAAGCCTCCACCTGGAGGACAATGCC |
| | CTCAAGGTCCTTCACAATGGCACCCTGGCTGAGTTGCAAGGTCTACCCCACATTAGGC |
| | TTTTCCTGGACAACAATCCCTGGGTCTGCGACTGCCACATGGCAGACATGGTGACCTG |
| | GCTCAAGGAAACAGAGGTAGTGCAGGGCAAAGACCGGCTCACCTGTGCATATCCGGAAA |
| | AAAATGAGGAATCGGGTCCTCTTGGAACTCAACAGTGCTGACCTGGACTGTGACCCGA |
| | TTCTTCCCCCATCCCTCACAAACCTCTTATGTCTTCCTGGGTATTGTTTTAGCCCTGAT |
| | AGGCGCTATTTTCCTCCTGGTTTTGTATTTGAACCGCAAGGGGATAAAAAAGTGGATG |
| | CATAACATCAGAGATGCCTGCAGGGATCACATGGAACCGTATCATTACAGATATGAAAA |
| | TCAATGCGGACCCCAGATTAACGAACCTCAGTTCTAACTCGGATGTCTGAGAAA |

| | |
|---|---|
| ORF Start: CTA at 1 | IORF Stop: TGA at 1150 |

| | | |
|---|---|---|
| SEQ ID NO: 46 | 383 aa | MW at 42319.8kD |

| | |
|---|---|
| NOV8a, | LALVLLGNVSSSSPTSSASSFSSSAPFLASAVSAQPPLPDQCPALCECSEAARTVKCV |
| CG56512-02 Protein | |
| Sequence | NRNLTEVPTDLPAYVRNLFLTGNQLAALNLSGSRLDEVRAGAFEHLPSLRQLDLSHNP |
| | LADLSPFAFSGSNASVSAPSPLVELILNHIVPPEDERQNRSFEGMVVAALLAGRALQG |
| | LRRLELASNIFLYLPRDVLAQLPSLRHLDLSNNSLVSLTYVSFRNLTHLESLHLEDNA |
| | LKVLHNGTLAELQGLPHIRVFLDNNPWVCDCHMADMVTWLKETEVVQGKDRLTCAYPE |
| | KNRNRVLLELNSADLDCDPILPPSLQTSYVFLGIVLALIGAIFLLVLYLNRKGIKKWM |
| | HNIRDACRDHMEGYHYRYEINADPRLTNLSSNSDV |

Further analysis of the NOV8a protein yielded the following properties shown in Table 8B.

TABLE 8B
Protein Sequence Properties NOV8a

| | |
|---|---|
| PSort | 0.4600 probability located in plasma membrane; 0.1000 prob- |
| analysis: | ability located in endoplasmic reticulum (membrane); 0.1000 |
| | probability located in endoplasmic reticulum (lumen); 0.1000 |
| | probability located in outside |
| SignalP | Cleavage site between residues 19 and 20 |
| analysis: | |

A search of the NOV8a protein against the Geneseq database, a proprietary database that contains sequences published in patents and patent publication, yielded several homologous proteins shown in Table 8C.

TABLE 8C
Geneseq Results for NOV8a

| | | Nov8a | Identities/ | |
|---|---|---|---|---|
| Geneseq | Protein/Organism/Length [Patent #, | Match | the Matched | Expect |
| Identifier | Date] | Residues | Region | Value |
| AAM93334 | Human polypeptide, SEQ ID NO: 2867 - | 1 . . . 383 | 383/400 (95%) | 0.0 |
| | Homo sapiens, 420 aa. [EP1130094- | 21 . . . 420 | 383/400 (95%) | |
| | A2, 05-SEP-2001] | | | |
| AAM93333 | Human polypeptide, SEQ ID NO: 2865 - | 1 . . . 346 | 346/363 (95%) | 0.0 |
| | Homo sapiens, 383 aa. [EP1130094- | 21 . . . 383 | 346/363 (95%) | |
| | A2, 05-SEP-2001] | | | |
| AAB83839 | Amino acid sequence of canine 5T4 | 1 . . . 383 | 333/400 (83%) | 0.0 |
| | protein - Canis sp, 420 aa. | 21 . . . 420 | 345/400 (86%) | |
| | [WO200136486-A2, 25-MAY-2001] | | | |
| AAY94351 | Canine 5T4 tumour-associated antigen - | 146 . . . 383 | 215/238 (90%) | e−123 |
| | Canis sp, 238 aa. [WO200029428-A2, | 1 . . . 238 | 221/238 (92%) | |
| | 25-MAY-2000] | | | |
| AAE13006 | Human leucine-rich repeat (LRR) | 28 . . . 307 | 94/321 (29%) | 2e−20 |
| | family member protein - Homo sapiens, | 43 . . . 361 | 130/321 (40%) | |
| | 713 aa. [WO200175105-A2, 11-OCT- | | | |
| | 2001] | | | |

In a BLAST search of public sequence databases, the NOV8a protein was found to have homology to the proteins shown in the BLASTP data in Table 8D.

TABLE 8D
Public BLASTP Results for NOV8a

| | | NOV8a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q13641 | 5T4 ONCOFETAL ANTIGEN | 1 . . . 383 | 383/400 (95%) | 0.0 |
| | PRECURSOR - Homo sapiens | 21 . . . 420 | 383/400 (95%) | |
| | (Human), 420 aa. | | | |
| Q9Z0L0 | 5T4 ONCOFETAL TROPHOBLAST | 1 . . . 383 | 322/407 (79%) | e−179 |
| | GLYCOPROTEIN PRECURSOR - | 21 . . . 426 | 345/407 (84%) | |
| | Mus musculus (Mouse), 426 aa. | | | |
| Q9QYD9 | 5T4 ONCOFETAL ANTIGEN | 1 . . . 383 | 318/407 (78%) | e−176 |
| | HOMOLOG - Rattus norvegicus | 21 . . . 426 | 340/407 (83%) | |
| | (Rat), 426 aa. | | | |
| CAD10322 | SEQUENCE 1 FROM PATENT | 28 . . . 307 | 94/321 (29%) | 6e−20 |
| | WO0175105 - Homo sapiens | 43 . . . 361 | 130/321 (40%) | |
| | (Human), 713 aa. | | | |
| Q9VK54 | KEK1 PROTEIN - Drosophila | 9 . . . 313 | 83/309 (26%) | 1e−19 |
| | melanogaster (Fruit fly), 880 aa. | 55 . . . 333 | 135/309 (42%) | |

PFam analysis indicates that the NOV8a protein contains the domains shown in the Table 8E.

TABLE 8E
Domain Analysis of NOV8a

| | | Identities/ | |
|---|---|---|---|
| | | Similarities | |
| Pfam Domain | NOV8a Match Region | for the Matched Region | Expect Value |
| LRRNT: domain 1 of 1 | 41 . . . 70 | 16/31 (52%) | 3.5e−08 |
| | | 25/31 (81%) | |
| LRR: domain 1 of 5 | 82 . . . 105 | 8/25 (32%) | 7.1 |
| | | 19/25 (76%) | |
| LRR: domain 2 of 5 | 106 . . . 129 | 10/25 (40%) | 0.04 |
| | | 18/25 (72%) | |
| LRR: domain 3 of 5 | 174 . . . 197 | 8/25 (32%) | 1.1 |
| | | 18/25 (72%) | |
| LRR: domain 4 of 5 | 198 . . . 221 | 14/25 (56%) | 0.0016 |
| | | 20/25 (80%) | |
| LRR: domain 5 of 5 | 222 . . . 245 | 8/25 (32%) | 0.36 |
| | | 20/25 (80%) | |
| LRRCT: domain 1 of 1 | 257 . . . 308 | 20/54 (37%) | 2.8e−18 |
| | | 48/54 (89%) | |

### Example 9

The NOV9 clone was analyzed, and the nucleotide and polypeptide sequences are shown in Table 9A.

TABLE 9A
NOV9 Sequence Analysis

| | SEQ ID NO:47 | 958 bp |
|---|---|---|
| NOV9a, | AAAACATGGCAGCCAAAGTGTTTGAGTCCACGGGTAAGTTTGGCTTGGCCTTACCTGT | |
| CG58180-01 DNA | TGCACGACTTGGCCTTAGCTGTTGCAGGAGACCTGTGAACTCTGCCTTATATAATGTG | |
| Sequence | GATGTTGGGCACAGAGCTGTCATCTTTGACAGATTCCAGGACAAACAGGACATTGTGG | |
| | TAGGCGACTCACTTTCTCATCCCATGGGTACAGAAACCAATTATCTTTGCCTTTCTCC | |
| | ACCACGTAATGTACCAATCATCACTGGTACCAAACATTTACAGAATGTCAATATCACA | |
| | CTGCGCATCATCTTCCAGCCTGTTGCTAGCCAGCTTCCTCGCATCTTCACCAGCATCG | |
| | GAGAGGACTATGATGAGCCTGTGCTGACGTACATCACGACCGAGATCCTCAAGTCAGT | |
| | GGTGGCTCGCTTTGATGCTGGAGAAGTTATCACTCAGAGAGAGCTGGTCTCCAGGCAG | |
| | CTGAGCAACGACCTTACGGAGCAAGCAGCCACATTTGGGCTCATCCTGGACGACGTGT | |
| | CCTTGACATATCTGACCTTTGGAAAGGAGTTCACAGAAGCAGTGGAAGCCAAACAGGT | |
| | GGCTCAGCAGGAAGCAGACAGGGCCAGATTTGTGAAGGAAAAGGCTGAGCAGCAGAAA | |
| | AAGGCTGAGCAGCAGAAAAAGGTTGAGCAGCAGAAAAAGGCAGCCGTGATCTCTGCTG | |
| | AGGGCGACTCCAAGGCAACCGAGCTGATTGCCAACTCACTGGCCACCGCGGGGGACGG | |
| | CCTGATGGAGCTGTGCAAGTTGCAAGCCGCGGAGTCTCGGAACATGACCTACCTGCCG | |
| | GCGGGGCAGTCCGCTCCTCCGGCTGCCCCATGAGGGCCCACCCTGCCTGCACCTCCGC | |
| | AGGCTGACTGGGCCACAGCCCCAATGATTCTTAACACTGCCTTACCCCCCTACCCCAG | |
| | AAATCACTGAAATTTCATAATTGGCTTAAA | |
| | ORF Start: ATG at 6 | ORF Stop: TGA at 843 |
| | SEQ ID NO:48 | 279 aa MW at 30368.2 kD |

| | |
|---|---|
| NOV9a, | MAAKVFESTGKFGLALAVAGLGLSCCRRPVNSALYNVDVGHRAVIFDRFQDKQDIVVG |
| CG58180-01 Protein | DSLSHPMGTETNYLCLSPPRNVPIITGSKDLQNVNITLRIIFQPVASQLPRIFTSIGE |
| Sequence | DYDEPVLTYITTEILKSVVARFDAGEVITQRELVSRQVSNDLTEQAATFGLILDDVSL |
| | TYLTFGKEFTEAVEAKQVAQQEAERARFVKEKAEQQKKAEQQKKVEQQKKAAVISAEG |
| | DSKATELIANSLATAGDGLMELCKLEAAESRNNTYLPAGQSAPPAAP |

Further analysis of the NOV9a protein yielded the following properties shown in Table 9B.

TABLE 9B
Protein Sequence Properties NOV9a

| | |
|---|---|
| PSort | 0.3700 probability located in outside; 0.1900 probability |
| analysis: | located in lysosome (lumen); 0.1000 probability located in |
| | endoplasmic reticulum (membrane); 0.1000 probability |
| | located in endoplasmic reticulum (lumen) |
| SignalP | Cleavage site between residues 33 and 34 |
| analysis: | |

A search of the NOV9a protein against the Geneseq database, a proprietary database that contains sequences published in patents and patent publication, yielded several homologous proteins shown in Table 9C.

TABLE 9C
Geneseq Results for NOV9a

| | | NOV9a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAG73845 | Human colon cancer antigen protein | 1 . . . 273 | 216/284 (76%) | e−104 |
| | SEQ ID NO:4609 - Homo sapiens, 279 | 8 . . . 272 | 230/284 (80%) | |
| | aa. [WO200122920-A2, 05-APR-2001] | | | |
| AAB43874 | Human cancer associated protein | 1 . . . 273 | 216/284 (76%) | e−104 |
| | sequence SEQ ID NO:1319 - Homo | 8 . . . 272 | 230/284 (80%) | |
| | sapiens, 279 aa. [WO200055350-A1, | | | |
| | 21-SEP-2000] | | | |
| AAW54352 | Heat shock 27 kD protein and | 1 . . . 273 | 216/284 (76%) | e−104 |
| | prohibitin (admixture) - Homo sapiens, | 200 . . . 464 | 230/284 (80%) | |
| | 1998] | | | |
| AAR42215 | Human prohibitin - Homo sapiens, 272 | 1 . . . 273 | 216/284 (76%) | e−104 |
| | aa. [JP05271294-A, 19-OCT-1993] | 1 . . . 265 | 230/284 (80%) | |
| AAR13466 | Prohibitin - Rattus rattus, 272 aa. | 1 . . . 273 | 215/284 (75%) | e−104 |
| | [US7612674-A, 16-JUL-1991] | 1 . . . 265 | 230/284 (80%) | |

In a BLAST search of public sequence databases, the NOV9a protein was found to have homology to the proteins shown in the BLASTP data in Table 9D.

TABLE 9D
Public BLASTP Results for NOV9a

| | | NOV9a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96CH8 | HYPOTHETICAL 22.1 KDA | 65 . . . 262 | 196/198 (98%) | e−104 |
| | PROTEIN - Homo sapiens (Human), | 1 . . . 198 | 197/198 (98%) | |
| | 201 aa. | | | |
| P35232 | Prohibitin - Homo sapiens (Human), | 1 . . . 273 | 216/284 (76%) | e−104 |
| | 272 aa. | 1 . . . 265 | 230/284 (80%) | |
| P24142 | Prohibitin (B-cell receptor associated | 1 . . . 273 | 215/284 (75%) | e−103 |
| | protein 32) (BAP 32) - Mus musculus | 1 . . . 265 | 230/284 (80%) | |
| | (Mouse), and, 272 aa. | | | |
| Q9VIZ4 | LETHAL (2) 37CC PROTEIN - | 1 . . . 273 | 159/284 (55%) | 7e−75 |
| | Drosophila melanogaster (Fruit fly), | 1 . . . 265 | 200/284 (69%) | |
| | 276 aa. | | | |
| Q9BKU4 | HYPOTHETICAL 30.0 KDA | 1 . . . 272 | 145/283 (51%) | 3e−64 |
| | PROTEIN - Caenorhabditis elegans, | 1 . . . 267 | 188/283 (66%) | |
| | 275 aa. | | | |

PFam analysis indicates that the NOV9a protein contains the domains shown in the Table 9E.

TABLE 9E
Domain Analysis of NOV9a

| | | Identities/ | |
|---|---|---|---|
| | NOV9a | Similarities for | Expect |
| Pfam Domain | Match Region | the Matched Region | Value |
| Band_7: domain 1 of 1 | 12 . . . 211 | 46/213 (22%) | 1.3e−42 |
| | | 162/213 (76%) | |

### Example 10

The NOV10 clone was analyzed, and the nucleotide and polypeptide sequences are shown in Table 10A.

TABLE 10A
NOV10 Sequence Analysis

| | SEQ ID NO:49 | 2482 bp |
|---|---|---|
| NOV 10a, | CGGCCACATAAOATATTATCTACACAAAAGCTTATTTAGGAATGACCAATGAGCTCAA | |
| CG59 199-01 DNA | CTTAAATTTATTAGAAATCTGATTAGCATTATAAATCAACTGACTTTTAGGAGCATAG | |
| Sequence | GCTTTGGCGCTACAGGGATCTGGCTTGAACCTCATTGAGGCCACTGATTGGTGAGTGA | |
| | CCTTAAGCAAGTCTCTTTACCTTGTTAGGGCACAATTTCCTCTCGGAATTGTGAAATG | |
| | GGTTTAATTACACCAGCTTACATCCCAACCATCAGCAAGTCCTCTTGTATGTAACCAT | |
| | CTCCAACACAGAAAGGGGATCTGCCCATGTTCTACCATCCTCACCTTTCTCCAAGCCA | |
| | CTACAACCTCTCACAACAGCCTTCCAATTGGTCTCCCTAAACTCTTATCCCCCCTACA | |
| | CCCCGTTCTCAGTCCTCAGTCCTTGCCCTAGGCTGGTAGCCCACTCCTTGCCCGCCCC | |
| | CCGCCTTCCTCCCATCTCCCCCTCCTCTCCCCGGCCCCCAGCACCTTCTGCATCCCAG | |
| | CCTACCTAGCCTACTCCTCCTCTTCCTGGCCCTCTTCCCCAGGCTCCAGGCTGGGGGG | |
| | TGCTCGCGTCTCCCCTGTAGGCCAGAGCAGCCCCAAGTTCTGGGGGCGGTCGGCCTGC | |
| | TGCTTTATCCCCATGGCCCTGCCATCACTTCTGCTGTTGGTGGCAGCCCTGGCAGGTG | |
| | GGGTGCGTCCTCCCGGGGCGCGGAACCTGACGCTGGCGGTGGTGCTGCCAGAACACAA | |
| | CCTGAGCTATGCCTGGCCCTGCCCACCGGTGGGACCCGCTGTGGCACTAGCTGTGGAG | |
| | GCTCTGGGCCGGGCACTGCCCGTGGACCTGCGGTTTGTCAGCTCCGAACTGGAAGGCG | |
| | CCTGCTCTGAGTACCTGGCACCGCTGAGCGCTGTGGACCTCAAGCTGTACCATGACCC | |
| | CGACCTGCTGTTAGGTCCCGGTTGCGTGTACCCTGCTGCCTCTCTCGCCCGCTTTGCC | |
| | TCCCACTGGCGCCTTCCCCTGCTGACTGCGGGTGCTGTGGCCTCTGGTTTTTCGGCTA | |
| | AGAATGACCATTATCGTACCCTGGTTCGCACTGGCCCCTCTGCTCCCAAGCTGGGTGA | |
| | GTTTGTGGTGACACTACACGGGCACTTCAATTGGACTGCCCGTGCTGCCTTGCTCTAC | |
| | CTGGATGCTCGCACAGATGACCGGCCTCACTACTTCACCATCGAGGGCGTCTTTGAGG | |
| | CCCTGCAGGCCAGCAACCTCAGTGTGCAGCACCAGGTGTATGCCCGAGAGCCAGGGGG | |
| | CCCCGAGCAGGCCACCCACTTCATCCGCGCCAACGGGCGCATTGTGTATATCTGCGGC | |
| | CCTCTGGAGATGCTGCATGAGATCCTGCTTCAGGCCCAGAGGGAGAATCTGACCAATG | |
| | GGGATTATGTCTTCTTTTACCTGGATGTCTTTGGGGAGAGTCTCCGTGCAGGCCCCAC | |
| | AAGTGATACAGGCCGGCCCTGGCAGGACAATCGCACCCGGGAACAGGCCCAGGCCCTC | |
| | AGAGAGGCCTTTCAGACTGTATTGGTGATCACGTACCGAGAACCCCCAAATCCTGAGT | |
| | ATCAGGAATTCCAGAATCGTCTGCTGATAAGAGCCCGGGAAGACTTTGGTGTGGAGCT | |
| | GGCCCCTTCCCTGATGAACCTCATCGCTGGCTGCTTCTATGATGGGATCCTGCTATAT | |
| | GCTGAAGTCCTGAATGAGACAATACAGGAAGGAGGCACCCGGGAGGATGGACTTCGAA | |
| | TTGTGGAAAAGATGCAGGGACGAAGATATCACGGAGTAACTGGGCTGGTTGTCATGGA | |
| | CAAGAACAATGACCGAAATGGTCAACGCCATGCACCAGAAATTGCTCGTATGGCCCTA | |
| | GCATTACTAGATGCAGTTTCTTCCTTTCGCATCCGCCACCGACCCCATGACCAGCTGA | |
| | GGCTACGCATAGGGGTCCATACTGGGCCAGTCTGTGCTCGGGTTGTTCGCCTGAAGAT | |
| | GCCCCGTTATTGTCTTTTTGGAGACACAGTGAACACTGCTTCTCGAATGGAGTCTAAT | |
| | GGTCAAGCGCTGAAGATCCATGTCTCCTCTACCACCAAGGATGCCCTAGATGAGCTAG | |
| | GATGCTTCCAGCTAGAGCTTCGGGGGGATGTGGAAATGAAGGGAAAAGGAAAGATGCG | |
| | AACATACTGGCTCTTAGCAGAGCCGAAAGGACCTCCTGGACTCCTGTAAACCCCCATT | |
| | CTTTCCAAGTCAGATACTCTTCTGCTGCTGGTACCTGCCTGGGCAATCGCCACCATGT | |
| | CTGCACACACCAGAAATGGACATTTTCATATGCAATCGAAAACAGCCACAAAAAAACC | |
| | TACCTTATATGGAAGTTGTAGCCCTCTGCAGCTCAGCCCTGTACATATATCTGTCCCT | |
| | CTCTGGCTTGGTCCCCTTCCTCCCTACTTTCTGTAAATATCTGTATCTAAACCAGAAT | |
| | ATTTTGGTCAAATATAAAACAATAATAAAAAAAGTTCTGATGTCAT | |
| | ORF Start: ATG at 651 | ORF Stop: TAA at 2193 |
| | SEQ ID NO:50 | 514 aa MW at 56836.4 kD |

| | |
|---|---|
| NOV 10a, | MALPSLLLLVAALAGGVRPPGARNLTLAVVLPEHNLSYAWAWPRVGPAVALAVEALGR |
| CG59199-01 Protein | ALPVDLRFVSSELEGACSEYLAPLSAVDLKLYHDPDLLLGPGCVYPAASVARFASHWR |
| Sequence | LPLLTAGAVASGFSAKNDHYRTLVRTGPSAPKLGEFVVTLHGHFNWTARAALLYLDAR |
| | TDDRPHYFTIEGVFEALQGSNLSVQHQVYAREPGGPEQATHFIRANCRIVYICGPLEM |
| | LHEILLQAQRENLTNGDYVFFYLDVFCESLRAGPTSDTGRPWQDNRTREQAQALREAF |
| | QTVLVITYREPPNPEYQEFQNRLLIRAREDFGVELGPSLMNLIAGCFYDGILLYAEVL |
| | NETIQEGGTREDGLRIVEKMQGRRYHGVTGLVVNDKNNDRNGQRHAPEIARMALALLD |
| | AVSSFRIRHRPHDQLRLRIGVHTGPVCAGVVGLKMPRYCLFGDTVNTASRMESNGQAL |
| | KIHVSSTTKDALDELGCFQLELRGDVEMKCKGKMRTYWLLGERKGPPGLL |

Further analysis of the NOV10a protein yielded the following properties shown in Table 10B.

TABLE 10B
Protein Sequence Properties NOV10a

| | |
|---|---|
| PSort | 0.8650 probability located in lysosome (lumen); 0.5517 prob- |
| analysis: | ability located in outside; 0.1000 probability located in |
| | endoplasmic reticulum (membrane); 0.1000 probability |
| | located in endoplasmic reticulum (lumen) |
| SignalP | Cleavage site between residues 17 and 18 |
| analysis: | |

A search of the NOV10a protein against the Geneseq database, a proprietary database that contains sequences published in patents and patent publication, yielded several homologous proteins shown in Table 10C.

TABLE 10C
Geneseq Results for NOV10a

| | | NOV10a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAR10867 | NPRB (Pro655, Glu656, Leu663, | 1 . . . 388 | 386/388 (99%) | 0.0 |
| | Phe664, Ala682) - Homo sapiens, 1047 | 1 . . . 388 | 386/388 (99%) | |
| | aa. [WO9100292-A, 10-JAN-1991] | | | |
| AAR10399 | Human Natriuretic Peptide Receptor B - | 1 . . . 388 | 386/388 (99%) | 0.0 |
| | Homo sapiens, 1047 aa. [WO9100292- | 1 . . . 388 | 386/388 (99%) | |
| | A, 10-JAN-1991] | | | |
| AAR38863 | GC-B - Rattus rattus, 1025 aa. | 23 . . . 388 | 352/366 (96%) | 0.0 |
| | [US5237051-A, 17-AUG-1993] | 1 . . . 366 | 357/366 (97%) | |
| AAR38862 | GC-A - Rattus rattus, 1029 aa. | 24 . . . 389 | 169/377 (44%) | 7e−83 |
| | [US5237051-A, 17-AUG-1993] | 2 . . . 373 | 227/377 (59%) | |
| ABB11783 | Human ANP-A receptor homologue, | 6 . . . 388 | 171/397 (43%) | 2e−79 |
| | SEQ ID NO:2153 - Homo sapiens, | 30 . . . 418 | 227/397 (57%) | |
| | 1075 aa. [WO200157188-A2, 09-AUG- | | | |
| | 2001] | | | |

In a BLAST search of public sequence databases, the NOV10a protein was found to have homology to the proteins shown in the BLASTP data in Table 10D.

TABLE 10D
Public BLASTP Results for NOV10a

| | | NOV10a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P20594 | Atrial natriuretic peptide receptor B | 1 . . . 388 | 386/388 (99%) | 0.0 |
| | precursor (ANP-B) (ANPRB) (GC-B) | 1 . . . 388 | 386/388 (99%) | |
| | (Guanylate cyclase) (EC 4.6.1.2) (NPR-B) | | | |
| | (Atrial natriuretic peptide B-type receptor) - | | | |
| | Homo sapiens (Human), 1047 aa. | | | |
| P16067 | Atrial natriuretic peptide receptor B | 1 . . . 388 | 376/388 (96%) | 0.0 |
| | precursor (ANP-B) (ANPRB) (GC-B) | 1 . . . 388 | 382/388 (97%) | |
| | (Guanylate cyclase) (EC 4.6.1.2) (NPR-B) | | | |
| | (Atrial natriuretic peptide B-type receptor) - | | | |
| | Rattus norvegicus (Rat), 1047 aa. | | | |
| P46197 | Atrial natriuretic peptide receptor B | 1 . . . 388 | 376/388 (96%) | 0.0 |
| | precursor (ANP-B) (ANPRB) (GC-B) | 1 . . . 388 | 381/388 (97%) | |
| | (Guanylate cyclase) (EC 4.6.1.2) (NPR-B) | | | |
| | (Atrial natriuretic peptide B-type receptor) - | | | |
| | Bos taurus (Bovine), 1047 aa. | | | |
| Q9PWG9 | GUANYLYL CYCLASE-2 - Xenopus | 25 . . . 389 | 233/372 (62%) | e−133 |
| | laevis (African clawed frog), 1082 aa. | 59 . . . 425 | 291/372 (77%) | |
| Q9Y117 | C-TYPE NATRIURETIC PEPTIDE | 6 . . . 389 | 197/395 (49%) | e−105 |
| | RECEPTOR PRECURSOR - Squalus | 8 . . . 398 | 258/395 (64%) | |
| | acanthias (Spiny dogfish), 1056 aa. | | | |

PFam analysis indicates that the NOV10a protein contains the domains shown in the Table 10E.

TABLE 10E
Domain Analysis of NOV10a

| | | Identities/ | |
|---|---|---|---|
| | | Similarities | |
| | NOV10a | for the | |
| | Match | Matched | Expect |
| Pfam Domain | Region | Region | Value |
| ANF_receptor: domain 1 of 1 | 21 . . . 391 | 121/427 (28%) | 5.9e−123 |
| | | 322/427 (75%) | |
| guanylate_cyc: domain 1 of 1 | 376 . . . 505 | 65/157 (41%) | 6.8e−54 |
| | | 110/157 (70%) | |

### Example 11

The NOV11 clone was analyzed, and the nucleotide and polypeptide sequences are shown in Table 11A.

TABLE 11A
NOV11 Sequence Analysis

| | SEQ ID NO:51 | 2372 bp |
|---|---|---|
| NOV11a, | TCCCATTTGACTCCATTTTCTTTGGTCAAAGGCTTCCTTATTCATGGGACCTGCCTGG | |
| CG59249-01 DNA | GTCCAGGACCCCTTGACAGGTGCTCTCTGGCTGCCTGTCCTCTGGGCACTCTTGTCCC | |
| Sequence | AGGTCTATTGTTTTCATGACCCACCAGGATGGCGCTTCACTTCCTCAGAAATTGTGAT | |
| | CCCCAGGAAAGTGCCCCACAAGAGGGGTGGAGTTGAGATGCCAGACCAGCTCTcTTAC | |
| | AGCATGCGTTTCCGGGGCCAAAGACACGTGATTCACATGAAGCTCAAGAAGAACATGA | |
| | TGCCCAGACATTTACCTGTTTTTACTGATAATGACCAAGGGGCCATGCAGGAGAACTA | |
| | CCCTTTTGTCCCACGAGACTGTTACTATGACTGCTACCTGGAAGGGGTTCCTGGGTCT | |
| | AGCATGCGTTTCCGGGGCCAAAGACACGTGATTCACATGAAGCTCAAGAAGAACATGA | |
| | TGACTTACGAAATCAAACACCTGGAGGCTTCTTCCAAATTTGAGCATGTAGTATCTCT | |
| | GCTTGTGTCAGAAGAAAGACCAGGAGAGGCTAGTAGATGTAAGACTGAAGGGCAAGAG | |
| | ATAGATCAAGAATCTGAAAAGGTAAAACTGGCTGAAACTCCCAGAGCAGGCCACGTTT | |
| | ATTTGTGGACGCATCATAGAAAAAACTTGAAAATTCACTACACGGTTACTCGTGGATT | |
| | ATTCATGCGGAACCCTAATGTGTCACATATAATAGAGAATGTAGTGATTATTAACAGC | |
| | ATCATACATACCATTTTCAAACCAGTTTATTTAAATGTCTATATATGTGTTTTGTGCA | |
| | TATGGAATCAAAAGGATGCACTACTATTTTCTGCTAGCAGGCCAGGCCACGTTGCTGT | |
| | AGAACTGTTTGGTGTGTCGAAATATCACAATTTGTATTCACAAATTTCACATGATACC | |
| | TCAGTTGTTTTTACATCAAATCGACTTGGAAACAGTGAGTGTTATGCCAGCTTTGATG | |
| | GAATATGCACCCCCAACTGGGGAGCAATGTTTGTGTATATAATGAGGTATCACCTATT | |
| | TAGGGGGGCATGTGTTACAGCACATGCACTAGGTCATAACATGGGCTTGAGACATGAT | |
| | TCTGTTGGTTGTTATTGTTTTCGACGAACCAACTGTCTCATGAGCAATTGTTCTTATG | |
| | AGATAATTCAACGCAAGTTTAATCAATGGGATCCTTGTTTGAGTGCTCCAAATGTTCC | |
| | ATACACTAATTTTCCATACGTAGCTCCTCGTTGTGGAGACAAGATCAAAAATCAGAGG | |
| | GAAGAATGTGACTGTGGCTCCCTTAAAGATTGTGCCAGTGATAGATGTTGTGAGACCT | |
| | CTTGTACCCTTTCTCTTGGCAGTGTTTGCAATACAGGACTTTGCTGCCATAAGTGTAA | |
| | ATATGCTGCCCCTGGAGTGGTTTGCAGAGACTTGGGTGGTATATGTGATCTACCGGAA | |
| | TACTGTGATGGGAAAAAGGAAGAGTGTCCAAATGACATCTACATCCAGGATGGAACCC | |
| | CATGTTCAGCAGTATCTGTTTGTATAACAGGAAACTGCAGTGACCGTGATATGCAGTG | |
| | TCAAGCCCTTTTTGGCTACCAAGTGAAACACCGTTCCCCAGCGTGCTATCGAAAATTG | |
| | AATAGGATTGGTAACCGATTTGGAAACTGTGGGGTTATTCTACGGCGACCGGGAAGTA | |
| | CACCTTTTCCATGTGAAGAAGATGATGTTTTTTGTGGAATGTTGCACTGTAGCGGTGT | |
| | CAGCCACATTCCCGGTGGAGGTGAGCACACTACATTTTGTAATATATTAGTACACGAC | |
| | ATAAAAGAAGAAAAATGCTTTGGCTATGAAGCACACCAGGGGACAGACTTGCCAGAAA | |
| | TGGGGCTGGTAGTGGATGGTGCAACCTGTGGCCCAGGGAGCTACTGTCTTAAACGCAA | |
| | TTGTACTTTTTATCAAGACCTGCATTTTGAGTGTGATCTTAAAACATGCAATTACAAA | |
| | GGAGTATGTAACAACAAAAAACATTGTCATTGTCTGCATGAGTGGCAACCACCAACAT | |
| | GTGAACTGAGAGGAAAAGGAGGTAGTATAGATAGTGGCCCTCTACCTGACAAACAATA | |
| | TCGTATTGCAGGCAGCATACTTGTAAATACAAACCGAGCACTAGTTTTAATATGTATT | |
| | CGTTACATCCTTTTTGTGGTTTCGCTTCTCTTTGGTGGCTTTTCACAAGCAATACAAT | |
| | GTTAGGGAAGAGAAAGCAAAAGAGCCCACACAATGGAGTAAATTACATTGACACTTAC | |
| | TGGCAGATATAATCAATAGTCACTCTGACAATTACATCATCTTTTAGCAATTCTCATG | |
| | TCATCTTGAAATAAAATCACTTGGCAATTTAAAAAGGTCTGTGTGTTTAAAT | |
| | ORF Start: ATG at 44 | ORF Stop: TAG at 2207 |
| | SEQ ID NO:52 | 721 aa MW at 81098.2 kD |
| NOV11a, | MGPAWVQDPLTGALWLPVLWALLSQVYCFHDPPGWRFTSSEIVIPRKVPHKRGGVEMP | |
| CG59249-01 Protein | DQLSYSMRFRGQRHVIHMKLKKNMMPRHLPVFTDNDQGANQENYPFVPRDCYYDCYLE | |
| Sequence | GVPGSAATLDTCRGGLHGMLQVDDLTYEIKHLEASSKFEHVVSLLVSEERPGEASRCK | |
| | TEGEEIDQESEKVKLAETPRAGHVYLWRHHRKNLKIHYTVTRGLFMRNPNVSHIIENV | |
| | ISHDTSVVFTSNRLGNSECYASFDGICTPNWGANFVYIMRYHLFRGACVTAHALGHNM | |
| | GLRHDSVGCYCFRRTNCLMSNCSYEIIQRKFNQWDPCLSAPNVPYTNFPYVAPRCGDK | |
| | IKNQREECDCGSLKDCASDRCCETSCTLSLGSVCNTGLCCHKCKYAAPGVVCRDLGGI | |
| | CDLPEYCDGKKEECPMDTYIQDGTPCSAVSVCIRCNCSDRDMQCQALFGYQVKDGSPA | |
| | CYRKLNRIGNRFGNCGVILRRGGSRPFPCEEDDVFCGMLHCSGVSHIPGGGEHTTFCN | |
| | ILVHDIKEEKCFGYEAHQGTDLPEMGLVVDGATCGPGSYCLKRNCTFYQDLHFECDLK | |
| | TCNYKGVCNNKKHCHCLHEWQPPTCELRGKGGSIDSGPLPDKQYRIAGSILVNTNRAL | |
| | VLICIRYILFVVSLLFGGFSQAIQC | |
| | SEQ ID NO:53 | 2429 bp |
| NOV11b, | TCCCATTTGACTGCATTTTCTTTGGTCAAAGGCTTCCTTATTCATGGGACCTGCCTGG | |
| CG59249-02 DNA | GTCCAGGACCCCTTGACAGGTGCTCTCTGGCTGCCTGTCCTCTGGGCACTCTTGTCCC | |
| Sequence | AGGTCTATTGTTTTCATGACCCACCAGGATGGCGCTTCACTTCCTCAGAAATTGTGAT | |
| | CCCCAGGAAAGTGCCCCACAAGAGGGGTGGAGTTGAGATGCCAGACCAGCTCTCTTAC | |
| | AGCATGCGTTTCCGGGGCCAAAGACACGTGATTCACATGAAGCTCAAGAAGAACATGA | |
| | TGCCCAGACATTTACCTGTTTTTACTGATAATGACCAAGGGGCCATGCAGGAGAACTA | |
| | CCCTTTTGTCCCACCACACTCTTACTATGACTGCTACCTGGAAGGGGTTCCTGGGTCT | |
| | GCGGCCACATTGGACACCTGCCGTGGAGGTCTGCATGGCATGCTGCAGCTGGATGACT | |
| | TGACTTACGAAATCAAACCCCTGGAGGCTTCTTCCAAATTTGAGCATGTAGTATCTCT | |
| | GCTTGTGTCAGAAGAAACACCAGGAGAGGCTAGTGGATGTATGACTGAAGGGGAAGAG | |
| | ATAGATCAAGAATCTGAAAAGGTAAAACTGGCTGAAACTCCCAGAGCAGGCCACGTTT | |
| | ATTTGTGGAGGCATCATAGAAAAAACTTGAAAATTCACTACACGGTTACTCGTGGATT | |
| | ATTCATGCGGAACCCTAATGTGTCACATATAATAGAGAATGTAGTGATTATTAACAGC | |
| | ATCATACATACCATTTTCAAACCAGTTTATTTAAATGTCTATATATGTGTTTTGTGCA | |
| | TATGGAATCAAAAGGATGCAGTACTATTTTCTGCTAGCAGGCCGGCCCACGTTGCTGT | |
| | AGAACTGTTTGGTGTGTGGAAATATCACAATTTGTATTCAGAGATTTCACATGATGCC | |
| | TCAGTTGTTTTTACATCAAATCGACTTGGAAACAGTGAGTGTTATGCCAGCTTTGATG | |
| | GAATATGCACCCCCAACTGGGGAGCAATGTTTGTGTATATAATGAGGTATCACCTATT | |
| | TAGGGGGGCATGTCTTACAGCACATGCACTAGGTCATAACATGGGCTTGAGACATGAT | |
| | TCTGTTGGTTGTTATTGTTTTCGACGAACCAACTATCTCATGGCTCCTGTTCCTGATC | |
| | TTAATGATATGATGAGCAATTGTTCTTATGAGATAATTCAACGCAAGTTTAATCAATG | |
| | GGATCCTTGTTTGAGTGCTCCAAATGTTCCATACACTAATTTTCCATACGTAGCTCCT | |
| | CGTTGTGGAGACAAGATCAAAAATCAGAGGGAAGAATGTGACTGTGGCTCCCTTAAAG | |
| | ATTGTGCCAGTGATAGATGTTGTGAGACCTCTTGTACCCTTTCTCTTGGCAGTGTTTG | |
| | CAATACAGGACTTTGCTGCCATAAGTGTAAATATGCTGCCCCTGGAGTGGTTTGCAGA | |
| | GACTTGGGTGGTATATGTGATCTACCGGAATACTGTGATGGGAAAAAGGAAGAGTGTC | |
| | CAAATGACATCTACATCCAGGATGGAACCCCATGTTCAGCAGTATCTGTTTGTATAAG | |
| | AGGAAACTGCAGTGACCGTGATATGCAGTGTCAAGCCCTTTTTGGCTACCAAGTGAAA | |
| | GACGGTTCCCCAGCGTGCTATCGAAAATTGAATAGGATTGGTAACCGATTTGGAAACT | |
| | GTGGGGTTATTCTACGGCGAGGGGGAAGTAGACCTTTTCCATGTGAAGAAGATGATGT | |
| | TTTTTGTGGAATGTTGCACTGTAGCGGTGTCAGCCACATTCCCGGTGGAGGTGAGCAC | |
| | ACTACATTTTGTAATATATTAGTACACGACATAAAAGAAGAAAAATCCTTTGGCTATG | |
| | AAGCACACCAGGGGACAGACTTGCCAGAAATGGGGCTGGTAGTGGATGGTGCAACCTG | |
| | TGGCCCAGGGAGCTACTGTCTTAAACGCAATTGTACTTTTTATCAAGACCTGCATTTT | |
| | GAGTGTGATCTTAAAACATGCAATTACAAAGGAGTATGTAGCAACAAAAAACATTGTC | |
| | ATTGTCTGCATGAGTGGCAACCACCAACATGTGAACTGAGAGGAAAAGGAGGTAGTAT | |
| | AGATAGTCGCCCTCTACCTGACAAACAATATCGTATTGCAGGCAGCATACTTGTAAAT | |
| | ACAAACCGAGCACTAGTTTTAATATGTATTCGTTACATCCTTTTTGTGGTTTCGCTTC | |
| | TCTTTGGTGGCTTTTCACAAGCAATACAATGTTAGGGAAGAGAAAGGAAAAGAGCCCA | |
| | CACAATGGAGTAAATTACATTGACACTTACTGGGAGATATAATCAATAGTCACTCTGA | |
| | CAATTACATCATCTTTTAGCAATTCTGATGTCATCTTGAAATAAAATCACTTGGCAAT | |
| | TTAAAAAAAAAAAAAAAAAAAAAAAAAAAAAAAAAAAAAAAAAAAAAAAGG | |
| | ORF Start: ATG at 44 | ORF Stop: TAG at 2237 |
| | SEQ ID NO:54 | 731 aa MW at 82049.3 kD |

| | |
|---|---|
| NOV11b, | MGPAWVQDPLTGALWLPVLWALLSQVYCFHDPPGWRFTSSEIVIPRKVPHKRGGVEMP |
| CG59249-02 Protein | DQLSYSMRFRGQRHVIHMKLKKNMMPRHLPVFTDNDQGAMQENYPFVPRDCYYDCYLE |
| Sequence | GVPGSAATLDTCRGGLHGMLQVDDLTYEIKPLEASSKFEHVVSLLVSEERPGEASGCM |
| | TEGEEIDQESEKVKLAETPRAGHVYLWRHHRKNLKIHYTVTRGLFMRNPNVSHIIENV |
| | VIINSIIHTIFKPVYLNVYICVLCIWNQKDAVLFSASRPGHVAVELFGVWKYHNLYSE |
| | ISHDASVVFTSNRLGNSECYASFDGICTPNWGAMFVYIMRYHLFRGACVTAHALGHNM |
| | GLRHDSVGCYCFRRTNYLMAPVPDLNDMNSNCSYEIIQRKFNQWDPCLSAPNVPYTNF |
| | PYVAPRCGDKIKNQREECDCGSLKDCASDRCCETSCTLSLGSVCNTGLCCHKCKYAAP |
| | GVVCRDLGGICDLPEYCDGKKEECPNDIYIQDGTPCSAVSVCIRGNCSDRDMQCQALF |
| | GYQVKDGSPACYRKLNRIGNRFGNCGVILRRGGSRPFPCEEDDVFCGMLHCSGVSHIP |
| | GGGEHTTFCNILVHDIKEEKCFGYEAHQGTDLPEMGLVVDGATCGPGSYCLKRNCTFY |
| | QDLHFECDLKTCNYKGVCSNKKHCHCLHEWQPPTCELRGKGGSIDSGPLPDKQYRIAG |
| | SILVNTNRALVLICIRYILFVVSLLFGGFSQAIQC |

Sequence comparison of the above protein sequences yields the following sequence relationships shown in Table 11B.

TABLE 11B
Comparison of NOV11a against NOV11b and NOV11c.

| Protein | NOV11a Residues/ | Identities/ |
|---|---|---|
| Sequence | Match Residues | Similarities for the Matched Region |
| NOV11b | 1 . . . 721 | 698/731 (95%) |
| | 1 . . . 731 | 699/731 (95%) |

Further analysis of the NOV11a protein yielded the following properties shown in Table 11C.

TABLE 11C
Protein Sequence Properties NOV11a

| | |
|---|---|
| PSort: | 0.8056 probability located in plasma membrane; 0.2800 prob- |
| analysis: | ability located in endoplasmic reticulum (membrane); 0.2000 |
| | probability located in lysosome (membrane); 0.1000 |
| | probability located in endoplasmic reticulum (lumen) |
| SignalP | Cleavage site between residues 29 and 30 |
| analysis: | |

A search of the NOV11a protein against the Geneseq database, a proprietary database that contains sequences published in patents and patent publication, yielded several homologous proteins shown in Table 11D.

TABLE 11D
Geneseq Results for NOV11a

| | | NOV11a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAY17413 | sapiens, 726 aa. [WO9923228-A1, 14- | 33 . . . 714 | 378/704 (53%) | e−135 |
| | MAY-1999] | | | |
| AAY03223 | Amino acid sequence of the novel | 33 . . . 712 | 250/704 (35%) | e−134 |
| | metalloprotease ADAM 16a - Homo | 33 . . . 714 | 377/704 (53%) | |
| | sapiens, 726 aa. [WO9907856-A1, 18- | | | |
| | FEB-1999] | | | |
| AAB07741 | A snake venom protease (SVPH-1) | 29 . . . 681 | 250/673 (37%) | e−132 |
| | polypeptide variant SVPH-1c - Homo | 27 . . . 671 | 360/673 (53%) | |
| | sapiens, 820 aa. [WO200043525-A2, | | | |
| | 27-JUL-2000] | | | |
| AAB07740 | A snake venom protease (SVPH-1) | 29 . . . 681 | 250/673 (37%) | e−132 |
| | polypeptide variant SVPH-1b - Homo | 27 . . . 671 | 360/673 (53%) | |
| | sapiens, 787 aa. [WO200043525-A2, | | | |
| | 27-JUL-2000] | | | |
| AAB07739 | A snake venom protease (SVPH-1) | 29 . . . 681 | 250/673 (37%) | e−132 |
| | polypeptide variant SVPH-1a - Homo | 27 . . . 671 | 360/673 (53%) | |
| | sapiens, 766 aa. [WO200043525-A2, | | | |
| | 27-JUL-2000] | | | |

In a BLAST search of public sequence databases, the NOV11a protein was found to have homology to the proteins shown in the BLASTP data in Table 11E.

TABLE 11E
Public BLASTP Results for NOV11a

| | | NOV11a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q95LW7 | HYPOTHETICAL 52.8 KDA PROTEIN - | 271 . . . 719 | 390/459 (84%) | 0.0 |
| | Macaca fascicularis (Crab eating | 2 . . . 460 | 415/459 (89%) | |
| | macaque) (Cynomolgus monkey), 474 aa. | | | |
| Q60815 | ADAM 4 PROTEIN - Mus musculus | 279 . . . 719 | 241/454 (53%) | e−151 |
| | (Mouse), 473 aa (fragment). | 1 . . . 449 | 309/454 (67%) | |
| Q28484 | TESTICULAR METALLOPROTEASE- | 19 . . . 713 | 268/711 (37%) | e−137 |
| | LIKE, DISINTEGRIN-LIKE, | 19 . . . 710 | 378/711 (52%) | |
| | CYSTEINE-RICH PROTEIN IVA - | | | |
| | Macaca fascicularis (Crab eating | | | |
| | macaque) (Cynomolgus monkey), 732 aa. | | | |
| O43506 | ADAM 20 precursor (EC 3.4.24.-) (A | 33 . . . 712 | 250/704 (35%) | e−134 |
| | disintegrin and metalloproteinase domain | 33 . . . 714 | 377/704 (53%) | |
| | 20) - Homo sapiens (Human), 726 aa. | | | |
| Q9UKF5 | ADAM 29 precursor (A disintegrin and | 29 . . . 681 | 250/673 (37%) | e−131 |
| | metalloproteinase domain 29) - Homo | 27 . . . 671 | 360/673 (53%) | |
| | sapiens (Human), 820 aa. | | | |

PFam analysis indicates that the NOV11a protein contains the domains shown in the Table 11F.

TABLE 11F
Domain Analysis of NOV11a

| | | Identities/ | |
|---|---|---|---|
| | NOV11a | Similarities | |
| | Match | for the Matched | Expect |
| Pfam Domain | Region | Region | Value |
| Pep_M12B_propep: | 75 . . . 191 | 34/119 (29%) | 2.2e−29 |
| domain 1 of 1 | | 91/119 (76%) | |
| Reprolysin: domain 1 of 1 | 206 . . . 392 | 51/210 (24%) | 1.7e−06 |
| | | 115/210 (55%) | |
| disintegrin: domain 1 of 1 | 409 . . . 484 | 30/77 (39%) | 3.3e−15 |
| | | 50/77 (65%) | |
| EGF: domain 1 of 1 | 635 . . . 663 | 8/47 (17%) | 2.7 |
| | | 20/47 (43%) | |

### Example 12

The NOV12 clone was analyzed, and the nucleotide and polypeptide sequences are shown in Table 12A.

TABLE 12A
NOV12 Sequence Analysis

| | SEQ ID NO:55 | 3874 bp |
|---|---|---|
| NOV12a, | ACAACTGTGATGATCCACTAGCATCCCTGCTCTCTCCAATGGCTTTTTCCAGTTCCTC | |
| CG58577-01 DNA | AGACCTCACTGGCACTCACAGCCCAGCTCAACTCAACTGGAGAGTTGGAACTGGCGGT | |
| Sequence | TGGTCCCCACCAGATTCCAATGCTCAACAGTGGCTCCAGATGGACCTGGGAAACAGAG | |
| | TAGAGATTACAGCAGTGGCCACGCAGGGAAGATACGGAAGCTCTGACTGGGTGACGAG | |
| | TTACAGCCTGATGTTCAGTGACACAGGACGCAACTGGAAACAGTACAAACAAGAAGAC | |
| | AGCATCTGGACCTTTGCAGGAAACATGAATGCTGACAGCGTGGTGCACCACAAGCTAT | |
| | TGCACTCAGTGAGAGCCCGATTTGTTCGCTTTGTGCCCCTGGAATGGAATCCCAGTGG | |
| | GAAGATTGGCATCAGACTCGAGGTCTACGGATGTTCCTATAATGTTGCTGACTTTGAT | |
| | GGCCGAAGCTCACTTCTGTACAGGTTCAATCAGAAGTTGATGAGTACTCTCAAAGATG | |
| | TGATCTCCCTGAAGTTCAAGAGCATGCAAGGAGATGGGGTCCTGTTCCATGGAGAAGG | |
| | TCAGCGTGGAGACCACATCACCTTGGAACTCCAGAAGGGGAGGCTCGCCCTACACCTC | |
| | AATTTGGGTGACAGCAAAGCGCGGCTCAGCAGCAGCTTGCCCTCTGCCACCCTGCGCA | |
| | GCCTCCTGGATGACCACCACTGGCACTCGGTCCTCATTGAGCGGGTGGGCAAGCAGGT | |
| | GAACTTCACGGTGGACAAGCACACACAGCACTTCCGCACCAAGGGCGAGACGGATGCC | |
| | TTAGACATTGACTATGAOCTTAGTTTTGGAGGAATTCCACTACCAGGAAAACcTGGGA | |
| | CCTTTTTAAAGAAAAACTTCCATGGATGCATCGAAAACCTTTACTACAATGGAGTAAA | |
| | CATAATTGACCTGGCTAAGAGACGAAAGCATCAGATCTATACTGTGGGCAATGTCACT | |
| | TTTTCCTGCTCCGAACCACAGATTGTGCCCATCACATTTGTCAACTCCAGCGGCAGCT | |
| | ATTTGCTGCTGCCCGGCACCCCCCAAATTGATGGGCTCTCAGTGAGTTTCCAGTTTCG | |
| | AACATGGAACAAGGATGGTCTCCTTCTGTCCACAGAGCTGTCTGAGGGCTCGGGAACC | |
| | CTGCTGCTGAGCCTGGAGGGTGGAATCCTGAGACTCGTGATTCAGAAAATGACAGAAC | |
| | GCGTAGCTGAAATCCTCACAGGCAGCAACTTGAATGATCGCCTGTGGCACTCGGTTAG | |
| | CATCAACGCCAGGAGGAACCGCATCACGCTCACTCTGGATGATGAAGCAGCACCCCCG | |
| | GCTCCAGACAGCACTTGGGTGCAGATTTATTCTGGAAATAGCTACTATTTTGGAGGTT | |
| | GCCCCGACAATCTCACCGATTCCCAATGTTTAAATCCCATTAAGGCTTTCCAAGGCTG | |
| | CATGAGGCTCATCTTTATTGATAACCAGCCCAAGGACCTCATTTCAGTTCAGCAAGGT | |
| | TCCCTGGGCAATTTTAGTGATTTACACATTGATCTGTGTAGCATCAAAGACAGGTGTT | |
| | TGCCAAACTACTGTGAACATGGAAGGAAGTTGTTCCCAGTCCTGGACTACCTTTTCTA | |
| | TTGTAACTGCAGTGACACAAGTTACACTGGTGCCACCTGCCACAACTCCATCTACGAG | |
| | CAATCCTGCGAGGTGTACAGGCACCAGGGGAATACAGCCGGCTTCTTCTACATCGACT | |
| | CAGATGGCAGCGGCCCACTGGGACCTCTCCAGGTGTACTGCAATATCACTCAGGACAA | |
| | CATCTGGACATCAGTGCAGCACAACAATACAGAGCTGACCCGAGTGCGGGGCGCTAAC | |
| | CCTGAGAAGCCCTATGCCATGGCCTTGGACTACGGGGGCAGCATGGAACAGCTGGAGG | |
| | CCGTGATCGACGGCTCTGAGCACTGTGAGCAGGAGGTGGCCTACCACTGCAGGAGGTC | |
| | CCGCCTGCTCAACACGCCGGATGGAACACCATTTACCTGGTGGATTGGGCGGTCCAAT | |
| | GAAAGGCACCCTTACTGGGGAGGTTCCCCTCCTGGGGTCCAGCAGTGTGAGTGTGGCC | |
| | TAGACGAGAGCTGCCTGGACATTCAGCACTTTTGCAATTGCGACGCTGACAAGGAAAA | |
| | TGATACTGGCTTTCTTTCCTTCAAAGACCACTTGCCTGTCACTCAGATAGTTATCACT | |
| | GATACCGACAGATCAAACTCAGAAGCCGCTTGGAGAATTGGTCCCTTGCGTTGCTATG | |
| | GTGACCCACCCTTCTGGAACGCCGTCTCATTTTATACAGAAGCCTCTTACCTCCACTT | |
| | TCCTACCTTCCATGCGGAATTCAGTGCCGATATTTCCTTCTTTTTTAAAACCACAGCA | |
| | TTATCCGGAGTTTTCCTAGAAAATCTTGGCATTAAAGACTTCATTCGACTCGAAATAA | |
| | GCTCTCCTTCAGAGATCACCTTTGCCATCGATGTTGGGAATGGTCCTGTGGAGCTTGT | |
| | AGTCCAGTCTCCTTCTCTTCTGAATGACAACCAATGGCACTATGTCCGGGCTGAGAGG | |
| | AACCTCAAGGAGACCTCCCTGCAGGTGGACAACCTTCCAAGGAGCACCAGGGAGACGT | |
| | CGGAGGAGGGCCATTTTCGACTGCAGCTGAACAGCCAGTTGTTTGTAGGCGGAACGTC | |
| | ATCCAGACAGAAAGGCTTCCTAGGATGCATTCGCTCCTTACACTTGAATGGACAGAAA | |
| | ATGGACCTGGAAGAGAGGGCAAAGGTCACATCTGGAGTCAGGCCAGGCTGCCCCGGCC | |
| | ACTGCAGCAGCTACGGCAGCATCTGCCACAACGGGGGCAAGTGTGTGGAGAAGCACAA | |
| | TGGCTACCTGTGTGATTGCACCAATTCACCTTATGAAGGGCCCTTTTGCAAAAAAGAG | |
| | GTTTCTCCTGTTTTTGAGGCTGGCACGTCGGTTACTTACATGTTTCAAGAACCCTATC | |
| | CTGTGACCAAGAATATAAGCCTCTCATCCTCAGCTATTTACACAGATTCAGCTCCATC | |
| | CAAGGAAAACATTGCACTTAGCTTTGTGACAACCCAGGCACCCAGTCTTTTGCTCTTT | |
| | ATCAATTCTTCTTCTCAGGACTTCGTGGTTGTTCTGCTCTGCAAGAATGGAAGCTTAC | |
| | AGGTTCCCTATCACCTAAACAAGGAAGAAACCCATGTATTCACCATTGATGCAGATAA | |
| | CTTTGCTAACAGAAGGATGCACCACTTGAAGATTAACCGAGAGGGAAGAGAGCTTACC | |
| | ATTCAGGTACCTTCCTTACTTTCTCCTGCTTCAGCCAATATGGACCAGCAACTTCGAC | |
| | TCAGTTATAACTTCTCTCCGGAAGTAGAGTTCAGGGTTATAAGGTCACTCACCTTGGG | |
| | CAAAGTCACAGAGAATCTTGGTTTGGATTCTGAAGTTGCTAAAGCAAATGCCATGGGT | |
| | TTTGCTGGATGCATGTCTTCCGTCCAGTACAACCACATAGCACCACTGAAGGCTGCCC | |
| | TGCGCCATGCCACTGTCGCGCCTCTCACTGTCCATGGGACCTTGACGGAATCCAGCTG | |
| | TGGCTTCATGGTGGACTCAGATGTGAATGCAGTGACCACGGTGCATTCTTCATCAGAT | |
| | CCTTTTGGGAAGACAGATGAGCGGGAACCACTCACAAATGCTGTTCGAAGTGATTCGG | |
| | CAGTCATCCGAGGGGTGATAGCAGTGGTGATATTCATCATCTTCTGTATCATCGGCAT | |
| | CATGACCCGGTTCCTCTACCAGCACAAGCAGTCACATCGTACGAGCCAGATGAAGGAG | |
| | AAGGAATATCCAGAAAATTTGGACAGTTCCTTCAGAAATGAAATTGACTTGCAAAACA | |
| | CAGTGAGCGAGTGTAAACGGGAATATTTCATCTGAGAAACTGCAGG | |
| | ORF Start: AAC at 3 | ORF Stop: TGA at 3861 |
| | SEQ ID NO:56 | 1286 aa MW at 143343.7 kD |
| NOV12a, | NCDDPLASLLSPMAFSSSSDLTGTHSPAQLNWRVGTGGWSPADSNAQQWLQMDLGNRV | |
| CG58577-01 Protein | EITAVATQGRYGSSDWVTSYSLMFSDTGRNWKQYKQEDSIWTFAGNMNADSVVHHKLL | |
| Sequence | HSVRARFVRFVPLEWNPSGKIGMRVEVYGCSYNVADFDGRSSLLYRFNQKLMSTLKDV | |
| | ISLKFKSMQGDGVLFHGEGQRGDHITLELQKGRLALHLNLGDSKARLSSSLPSATLGS | |
| | LLDDQHWHSVLIERVGKQVNFTVDKHTQHFRTKGETDALDIDYELSFGGIPVPGKPGT | |
| | FLKKNFHGCIENLYYNCVNIIDLAKRRKHQIYTVGNVTFSCSEPQIVPITFVNSSGSY | |
| | LLLPGTPQIDGLSVSFQFRTWNKDGLLLSTELSEGSGTLLLSLEGGILRLVIQKNTER | |
| | VAEILTGSNLNDGLWHSVSINARRNRITLTLDDEAAPPAPDSTWVQIYSCNSYYFGGC | |
| | PDNLTDSQCLNPIKAFQGCMRLIFIDNQPKDLISVQQGSLGNFSDLHIDLCSIKDRCL | |
| | PNYCEHGRKLFPVLDYLFYCNCSDTSYTGATCHNSIYEQSCEVYRHQGNTAGFFYIDS | |
| | DGSGPLGPLQVYCNITEDKIWTSVQHNNTELTRVRGANPEKPYAMALDYGGSMEQLEA | |
| | VIDGSEHCEQEVAYHCRRSRLLNTPDGTPFTWWIGRSNERHPYWGGSPPGVQQCECGL | |
| | DESCLDIQHFCNCDADKENDTGFLSFKDHLPVTQIVITDTDRSNSEAAWRIGPLRCYG | |
| | DRRFWNAVSFYTEASYLHFPTFHAEFSADISFFFKTTALSGVFLENLGIKDFIRLEIS | |
| | SPSEITFAIDVGNGPVELVVQSPSLLNDNQWHYVRAERNLKETSLQVDNLPRSTRETS | |
| | EEGHFRLQLNSQLFVGGTSSRQKGFLGCIRSLHLNGQKMDLEERAKVTSGVRPGCPGH | |
| | CSSYGSICHNGGKCVEKHNGYLCDCTNSPYEGPFCKKEVSAVFEAGTSVTYMFQEPYP | |
| | VTKNISLSSSAIYTDSAPSKENIALSFVTTQAPSLLLFINSSSQDFVVVLLCKNGSLQ | |
| | VRYHLNKEETHVFTIDADNFANRRMHHLKINREGRELTIQVPSLLSPASANMDQQLRL | |
| | SYNFSPEVEFRVIRSLTLGKVTENLGLDSEVAKANANGFAGCMSSVQYNHIAPLKAAL | |
| | RHATVAPVTVHGTLTESSCGFMVDSDVNAVTTVHSSSDPFGKTDEREPLTNAVRSDSA | |
| | VIGGVIAVVIFIIFCIIGIMTRFLYQHKQSHRTSQMKEKEYPENLDSSFRNEIDLQNT | |
| | VSECKREYFI | |
| | SEQ ID NO:57 | 429 bp |
| NOV12b, | GGATCCCCACTAGCATCCCTGCTCTCTCCAATGGCTTTTTCCAGTTCCTCAGACCTCA | |
| 174307971 DNA | CTGGCACTCACAGCCCAGCTCAACTCAACTGGAGAGTTGGAACTGGCGGTTGGTCCCC | |
| Sequence | ACCAGATTCCAATGCTCAACAGTGGCTCCAGATGGACCTGGGAAACAGAGTAGAGATT | |
| | ACAGCAGTGGCCACGCAGGGAAGATACGGAAGCTCTGACTGGGTGACGAGTTACAGCC | |
| | TGATGTTCAGTGACACAGGACGCAACTGGAAACAGTACAAACAAGAAGACAGCATCTG | |
| | GACCTTTGCAGGAAACATGAATGCTGACAGCGTGGTGCACCACAAGCTATTGCACTCA | |
| | GTGAGAGCCCGATTTGTTCGCTTTGTGCCCCTGGAATCGAATCCCAGTGGGAAGATTG | |
| | GCATGAGAGTCGAGGTCCTCGAG | |
| | ORF Start: GGA at 1 | ORF Stop: al at 430 |
| | SEQ ID NO:58 | 143 aa MW at 15889.6 kD |
| NOV12b, | GSPLASLLSPMAFSSSSDLTGTHSPAQLNWRVGTGGWSPADSNAQQWLQMDLGNRVEI | |
| 174307971 Protein | TAVATQGRYGSSDWVTSYSLMFSDTGRNWKQYKQEDSIWTFAGNMNADSVVHHKLLHS | |
| Sequence | VRARFVRFVPLEWNPSGKIGMRVEVLE | |
| | SEQ ID NO:59 | 429 bp |
| NOV12c, | GGATCCCCACTAGCATCCCTGCTCTCTCCAATCGCTTTTTCCAGTTCCTCAGACCTCA | |
| 174307975 DNA | CTGGCACTCACAGCCCAGCTCAACTCAACTGGAGAGTTGGAACTGGCGGTTGGTCCCC | |
| Sequence | AGCAGATTCCAATGCTCAACAGTGGCTCCAGATGGACCTGGGAAACAGAGTAGAGATT | |
| | ACAGCAGTGGCCACGCAGGGAAGGTACGGAAGCTCTGACTGGGTGACGAGTTACAGCC | |
| | TGATGTTCAGTGACACAGGACGCAACTGGAAACAGTACAAACAAGAAGACAGCATCTG | |
| | GACCTTTGCAGGAAACATGAATGCTGACAGCGTCGTGCACCACAAGCTATTGCACTCA | |
| | GTGAGAGCCCGATTTGTTCGCTTTGTGCCCCTGGAATGGAATCCCAGTGGGAAGATTG | |
| | GCATOAGAGTCGAGGTCCTCGAG | |
| | ORF Start: GGA at 1 | ORF Stop: al at 430 |
| | SEQ ID NO:60 | 143 aa MW at 15889.6 kD |
| NOV12c, | GSPLASLLSPMAFSSSSDLTGTHSPAQLNWRVGTGGWSPADSNAQQWLQMDLGNRVEI | |
| 174307971 Protein | TAVATQGRYGSSDWVTSYSLMFSDTGRNWKQYKQEDSIWTFAGNMNADSVVHHKLLHS | |
| Sequence | VRARFVRFVPLEWNPSGKIGMRVEVLE | |
| | SEQ ID NO:61 | 429 bp |
| NOV12d, | GGATCCCCACTAGCATCCCTGCTCTCTCCAATCGCTTTTTCCAGTTCCTCAGACCTCA | |
| 174307979 DNA | CTGGCACTCACAGCCCAGCTCAACTCAACTGGAGAGTTGGAACTGGCGGTTGGTCCCC | |
| Sequence | AGCAGATTCCAATGCTCAACAGTGGCTCCAGATGGACCTGGGAAACAGAGTAGAGATT | |
| | ACAGCAGTGGCCACGCAGGGAAGATACGGAAGCTCTGACTGGGTGACGAGTTACAGCC | |
| | TGATGTTCAGTGACACAGGACGCAACTGGAAACAGTACAAACAAGAAGACAGCATCTG | |
| | GACCTTTGCAGGAAACATGAATGCTGACAGCGTGGTGCACCACAAGCTATTGCGCTCA | |
| | GTGAGAGCCCGATTTGTTCGCTTTGTGCCCCTGGAATGGAATCCCACTGGGAAGATTG | |
| | GCATGAGAGTCGAGGTCCTCGAG | |
| | ORF Start: GGA at 1 | ORF Stop: al at 430 |
| | SEQ ID NO:62 | 143 aa MW at 15908.7 kD |
| NOV12d, | GSPLASLLSPMAFSSSSDLTGTHSPAQLNWRVGTGGWSPADSNAQQWLQMDLGNRVEI | |
| 174307979 Protein | TAVATQGRYGSSDWVTSYSLMFSDTGRNWKQYKQEDSIWTFAGNMNADSVVHHKLLRS | |
| Sequence | VRARFVRFVPLEWNPSGKIGMRVEVLE | |
| | SEQ ID NO:63 | 429 bp |
| NOV12e, | GGATCCCCACTAGCATCCCTGCTCTCTCCAATGGCTTTTTCCAGTTCCTCAGACCTCA | |
| 174307983 DNA | CTGGCACTCACAGCCCAGCTCAACTCAACTGGAGAGTTGGAACTGGCGGTTGGTCCCC | |
| Sequence | AGCAGATTCCAATGCTCAACAGTGGCTCCAGATGGACCTCGGAAACAGAGTAGAGATT | |
| | ACAGCAGTGGCCACGCAGGGAAGATACGGAAGCTCTGACTGGGTGACGAGTTACAGCC | |
| | TGATGTTCAGTGACACAGGACGCAACTGGAAACAGTACAAACAGGAAGACAGCATCTG | |
| | GACCTTTGCAGGAAACATGAATGCTGACAGCGTGGTGCACCACAAGCTATTGCACTCA | |
| | GTGAGAGCCCGATTTGTTCGCTTTGTGCCCCTGGAATGGAATCCCAGTGGGAAGATTG | |
| | GCATGAGAGTCCAGGTCCTCCAG | |
| | ORF Start: GGA at 1 | ORF Stop: al at 430 |
| | SEQ ID NO:64 | 143 aa MW at 15889.6 kD |
| NOV12e, | GSPLASLLSPMAFSSSSDLTGTHSPAQLNWRVGTGGWSPADSNAQQWLQMDLGNRVEI | |
| 174307983 Protein | TAVATQGRYGSSDWVTSYSLMFSDTGRNWKQYKQEDSIWTFAGNMNADSVVHHKLLHS | |
| Sequence | VRARFVRFVPLEWNPSGKIGMRVEVLE | |
| | SEQ ID NO:65 | 1429 bp |
| NOV12f, | GGATCCCCACTAGCATCCCTGCTCTCTCCAATGGCTTTTTCCAGTTCCTCAGACCTCA | |
| 174307987 DNA | CTGGCACTCACAGCCCAGCTCAACTCAACTGGAGAGTTGGAACTGGCGGTTGGTCCCC | |
| Sequence | AGCAGATTCCAATGCTCAACAGTGGCTCCAGATGGACCTGGGAAACAGAGTAGAGATT | |
| | ACAGCAGTGGCCACGCGGGGAAGATACGGAAGCTCTGACTGGGTGACGAGTTACAGCC | |
| | TGATGTTCAGTGACACAGGACGCAACTGGAAACAGTACAAACAAGAAGACAGCATCTG | |
| | GACCTTTGCAGGAAACATGAATGCTGACACCGTGGTGCACCACGAGCTATTGCACTCA | |
| | GTGAGAGCCCGATTTGTTCGCTTTGTGCCCCTGGAATGGAATCCCAGTGGGAAGATTG | |
| | GCATGAGAGTCGAGGTCCTCGAG | |
| | ORF Start: GGA at 1 | ORF Stop: al at 430 |
| | SEQ ID NO:66 | 143 aa MW at 15918.6 kD |
| NOV12f, | GSPLASLLSPMAFSSSSDLTGTHSPAQLNWRVGTGGWSPADSNAQQWLQMDLGNRVEI | |
| 174307987 Protein | TAVATRGRYGSSDWVTSYSLMFSDTGRNWKQYKQEDSIWTFAGNMNADSVVHHELLHS | |
| Sequence | VRARFVRFVPLEWNPSGKIGMRVEVLE | |
| | SEQ ID NO:67 | 429 bp |
| NOV12g, | GGATCCCCACTAGCATCCCTGCTCTCTCCAATGGCTTTTTCCAGTTCCTCAGACCTCA | |
| 174307996 DNA | CTGGCACTCACAGCCCAGCTCAACTCAACTGGAGAGTTGGAACTGGCGGTTGGTCCCC | |
| Sequence | AGCAGATTCCAATGCTCAACAGTGGCTCCAGATGGACCTGGGAAACAGAGTAGAGATT | |
| | ACAGCAGTGGCCACGCAGGGAAGATACGGAAGCTCTGACTGGGTGACGAGTTACAGCC | |
| | TGATGTTCAGTGACACAGGACGTAACTGGAAACAGTACAAACAAGAAGACAGCATCTG | |
| | GACCTTTGCAGGAAACATGAATGCTGACAGCGTGGTGCACCACAAGCTATTGCACTCA | |
| | GTGAGAGCCCGATTTGTTCGCTTTGTGCCCCTGGAATGGAATCCCAGTGGGAAGATTG | |
| | GCATGAGAGTCGAGGTCCTCGAG | |
| | ORF Start: GGA at 1 | ORF Stop: al at 430 |
| | SEQ ID NO:68 | 143 aa MW at 15889.6 kD |
| NOV12g, | GSPLASLLSPMAFSSSSDLTGTHSPAQLNWRVGTGGWSPADSNAQQWLQMDLGNRVEI | |
| 174307996 Protein | TAVATQGRYGSSDWVTSYSLMFSDTGRNWKQYKQEDSIWTFAGNNNADSVVHHKLLHS | |
| Sequence | VRARFVRFVPLEWNPSGKIGMRVEVLE | |
| | SEQ ID NO:69 | 429 bp |
| NOV12h, | GGATCCCCACTAGCATCCCTGCTCTCTCCAATGGCTTTTTCCAGTTCCTCAGACCTCA | |
| 169894929 DNA | CTGGCACTCACAGCCCAGCTCAACTCAACTGGAGAGTTGGAACTGGCGGTTGGTCCCC | |
| Sequence | AGCACATTCCAATGCTCAACAGTGGCTCCAGATGGACCTGGGAAGCAGAGTAGAGATT | |
| | ACAGCAGTGGCCACGCAGGGAAGATACGGAAGCTCTGACTGGGTGACGAGTTACAGCC | |
| | TGATGTTCAGTGACACAGGACGCAACTGGAAACAGTACAAACAAGAAGACAGCATCTG | |
| | GACCTTTGCAGGAAACATCAACGCTGACAGCGTGGTGCACCACAAGCTATTGCACTCA | |
| | GTGAGAGCCCCGATTTGTTCGCTTTGTGCCCCTGGAATGGAATCCAGTGGGAAGATTG | |
| | GCATGAGAGTCGAGGTCCTCGAG | |
| | ORF Start: GGA at 1 | ORF Stop: al at 430 |
| | SEQ ID NO:70 | 143 aa MW at 15862.6 kD |

| | |
|---|---|
| NOV12h, | GSPLASLLSPMAFSSSSDLTGTHSPAQLNWRVGTCGWSPADSNAQQWLQMDLGSRVEI |
| 169894929 Protein | TAVATQGRYGSSDWVTSYSLMFSDTGRNWKQYKQEDSIWTFAGNNNADSVVHHKLLHS |
| Sequence | VRARFVRFVPLEWNPSGKIGMRVEVLE |

Sequence comparison of the above protein sequences yields the following sequence relationships shown in Table 12B.

TABLE 12B
Comparison of NOV12a against NOV12b through NOV12h.

| | NOV12a Residues/ | Identities/Similarities |
|---|---|---|
| Protein Sequence | Match Residues | for the Matched Region |
| NOV12b | 22 . . . 143 | 122/122 (100%) |
| | 20 . . . 141 | 122/122 (100%) |
| NOV12c | 22 . . . 143 | 122/122 (100%) |
| | 20 . . . 141 | 122/122 (100%) |
| NOV12d | 22 . . . 143 | 121/122 (99%) |
| | 20 . . . 141 | 121/122 (99%) |
| NOV12e | 22 . . . 143 | 122/122 (100%) |
| | 20 . . . 141 | 122/122 (100%) |
| NOV12f | 22 . . . 143 | 120/122 (98%) |
| | 20 . . . 141 | 122/122 (99%) |
| NOV12g | 20 . . . 141 | 122/122 (100%) |
| NOV12h | 22 . . . 143 | 121/122 (99%) |
| | 20 . . . 141 | 122/122 (99%) |

Further analysis of the NOV12a protein yielded the following properties shown in Table 12C.

TABLE 12C
Protein Sequence Properties NOV12a

| | |
|---|---|
| PSort | 0.7000 probability located in plasma membrane; 0.4467 prob- |
| analysis: | ability located in microbody (peroxisome); 0.3000 probability |
| | located in nucleus; 0.2000 probability located in |
| | endoplasmic reticulum (membrane) |
| SignalP | No Known Signal Sequence Indicated |
| analysis: | |

A search of the NOV12a protein against the Geneseq database, a proprietary database that contains sequences published in patents and patent publication, yielded several homologous proteins shown in Table 12D.

TABLE 12D
Geneseq Results for NOV12a

| | | NOV12a | | |
|---|---|---|---|---|
| | | Residues/ | Identities/ | |
| Geneseq | Protein/Organism/Length [Patent | Match | Similarities for the | Expect |
| Identifier | #, Date] | Residues | Matched Region | Value |
| AAE07293 | Human neurexin-like protein #12 - | 1 . . . 1286 | 1263/1291 (97%) | 0.0 |
| | Homo sapiens, 1298 aa. | 20 . . . 1298 | 1265/1291 (97%) | |
| | [WO200158938-A2, 16-AUG-2001] | | | |
| AAE07282 | Human neurexin-like protein #1 - | 1 . . . 1286 | 1261/1291 (97%) | 0.0 |
| | Homo sapiens, 1307 aa. | 29 . . . 1307 | 1263/1291 (97%) | |
| | [WO200158938-A2, 16-AUG-2001] | | | |
| AAE07283 | Human neurexin-like protein #2 - | 1 . . . 1286 | 1214/1291 (94%) | 0.0 |
| | Homo sapiens, 1259 aa. | 29 . . . 1259 | 1216/1291 (94%) | |
| | [WO200158938-A2, 16-AUG-2001] | | | |
| AAE07294 | Human neurexin-like protein #13 - | 105 . . . 1286 | 1159/1187 (97%) | 0.0 |
| | Homo sapiens, 1175 aa. | 1 . . . 1175 | 1161/1187 (97%) | |
| | [WO200158938-A2, 16-AUG-2001] | | | |
| AAE07291 | Human neurexin-like protein #10 - | 1 . . . 797 | 749/802 (93%) | 0.0 |
| | Homo sapiens, 839 aa. | 29 . . . 829 | 755/802 (93%) | |
| | [WO200158938-A2, 16-AUG-2001] | | | |

In a BLAST search of public sequence databases, the NOV12a protein was found to have homology to the proteins shown in the BLASTP data in Table 12E.

TABLE 12E
Public BLASTP Results for NOV12a

| | | NOV12a | Identities/ | |
|---|---|---|---|---|
| Protein | | Residues/ | Similarities for | |
| Accession | | Match | the Matched | Expect |
| Number | Protein/Organism/Length | Residues | Portion | Value |
| BAB83897 | CASPR5 - Homo sapiens (Human), | 1 . . . 1286 | 1262/1291 (97%) | 0.0 |
| | 1306 aa. | 29 . . . 1306 | 1264/1291 (97%) | |
| Q96MS7 | CDNA FLJ31966 FIS, CLONE | 1 . . . 925 | 912/930 (98%) | 0.0 |
| | NT2RP7007925, WEAKLY SIMILAR | 29 . . . 957 | 914/930 (98%) | |
| | TO HOMO SAPIENS CONTACTIN | | | |
| | ASSOCIATED PROTEIN (CASPR) | | | |
| | MRNA - Homo sapiens (Human), 963 | | | |
| | aa. | | | |
| AAL68839 | CELL RECOGNITION PROTEIN | 1 . . . 1285 | 747/1293 (57%) | 0.0 |
| | CASPR4 - Homo sapiens (Human), | 33 . . . 1310 | 961/1293 (73%) | |
| | 1311 aa. | | | |
| Q9C0A0 | Contactin associated protein-like 4 | 1 . . . 1285 | 747/1293 (57%) | 0.0 |
| | precursor (Cell recognition molecule | 30 . . . 1307 | 961/1293 (73%) | |
| | Caspr4) - Homo sapiens (Human), 1308 | | | |
| | aa. | | | |
| Q99P47 | Contactin associated protein-like 4 | 1 . . . 1285 | 725/1293 (56%) | 0.0 |
| | precursor (Cell recognition molecule | 32 . . . 1309 | 955/1293 (73%) | |
| | Caspr4) - Mus musculus (Mouse), 1310 | | | |
| | aa. | | | |

PFam analysis indicates that the NOV12a protein contains the domains shown in the Table 12F.

TABLE 12F
Domain Analysis of NOV12a

| | | Identities/ | |
|---|---|---|---|
| | NOV12a | Similarities | |
| | Match | for the Matched | Expect |
| Pfam Domain | Region | Region | Value |
| F5_F8_type_C: | 5 . . . 143 | 48/167 (29%) | 1e−41 |
| domain 1 of 1 | | 115/167 (69%) | |
| laminin_G: domain 1 of 4 | 179 . . . 311 | 40/168 (24%) | 3.6e−11 |
| | | 93/168 (55%) | |
| laminin_G: domain 2 of 4 | 366 . . . 495 | 40/161 (25%) | 3.5e−12 |
| | | 88/161 (55%) | |
| EGF: domain 1 of 2 | 521 . . . 554 | 22/47 (47%) | 3.5 |
| TSPN: domain 1 of 1 | 729 . . . 908 | 35/225 (16%) | 8.5 |
| | | 115/225 (51%) | |
| laminin_G: domain 3 of 4 | 788 . . . 910 | 44/164 (27%) | 4.5e−15 |
| | | 92/164 (56%) | |
| EGF: domain 2 of 2 | 929 . . . 963 | 14/47 (30%) | 0.0044 |
| | | 27/47 (57%) | |
| laminin_G: domain 4 of 4 | 1014 . . . 1085 | 20/87 (23%) | 0.0019 |
| | | 51/87 (59%) | |

### Example 13

The NOV13 clone was analyzed, and the nucleotide and polypeptide sequences are shown in Table 13A.

TABLE 13A
NOV13 Sequence Analysis

| | SEQ ID NO:71 | 14109 bp |
|---|---|---|
| NOV13a, | TGTCGCTCAACGGGATGCCCCTGTACAACGACAGCTTCCATGAGATCTCACACAAGGG | |
| CG59237-01 DNA | CCGCCGCCACACGCTGGTACTCAAGACCATCCAGCGGGCTCATGCCGGCATAGTACGC | |
| Sequence | GCCTCCTCCCTGAAGGTGTCGACCTCTGCCCGCCTGGAGGTCCGAGTGAAGCCCGTGG | |
| | TGTTCCTGAAGGCGCTGGATGACCTGTCCGCAGAGGAGCGCGGCACCCTGGCCCTGCA | |
| | GTGTGAAGTCTCTGACCCCGAGGCCCATGTGGTGTGGCGCAAAGATGGCGTGCAGCTG | |
| | GGCCCCAGTGACAAGTATGACTTCCTGCACACGGCGGGCACGCGGGGGCTCGTGGTGC | |
| | ATGACGTGAGCCCTGAAGACGCCGGCCTGTACACCTGCCACGTGGGCTCCGAGGAGAC | |
| | CCGGGCCCGGGTCCGCGTGCACGATCTGCACGTGGGCATCACCAACAGGCTGAAGACA | |
| | ATGGAGGTGCTGGAAGGGGAAAGCTGCAGCTTTGAGTGCGTCCTGTCCCACGAGAGTG | |
| | CCAGCGACCCGGCCATGTGGACAGTCGGTGGGAAGACAGTGGCCAGCTCCAGCCGCTT | |
| | CCAGGCCACACGTCAGGGCCGAAAATACATCCTGGTGGTCCGGGAGGCTGCACCAAGT | |
| | GATGCCGGGGAGGTGGTCTTCTCTGTGCGGGGCCTCACCTCCAAGGCCTCACTCATTG | |
| | TCAGAGAGAGGCCGGCCGCCATCATCAAGCCCCTGGAAGACCAGTGGGTGGCGCCAGG | |
| | GGAGGACGTGGAGCTGCGCTGTGAGCTGTCACGGGCCGGAACGCCCCTGCACTGGCTG | |
| | AAGGACAGGAAGGCCATCCGCAAGAGCCAGAAGTATGATGTGGTCTGCGACGGCACGA | |
| | TGGCCATGCTGGTCATCCGCGGGGCCTCGCTCAAGGACGCGGGCGAGTACACGTGTGA | |
| | GGTGCAGGCTTCCAAGAGCACAGCCAGCCTCCATGTGGAAGAAAAAGCAAACTGCTTC | |
| | ACAGAGGAGCTGACCAATCTGCACGTGCAGGACAAAGGCACAGCTGTGTTCACGTGCA | |
| | AGACGGAGCACCCCGCGGCCACAGTGACCTGGCGCAAGGGCCTCTTGGAGCTACGGGC | |
| | CTCAGGGAAGCACCAGCCCAGCCAGGAGGGCCTGACCCTGCGCCTCACCATCAGTGCC | |
| | CTGGAGAAGGCAGACAGCGACACCTATACCTGCGACATTGGCCAGGCCCAGTCCCGGG | |
| | CCCAGCTCCTAGTGCAAGGCCGGAGAGTGCACATCATCCAGGACCTGGAGGATGTGGA | |
| | TGTGCACGAGGGCTCCTCGGCCACCTTCCGTTCCCGGATCTCCCCGGCCAACTACGAG | |
| | CCTGTGCACTGGTTCCTGGACAAGACACCCCTGCATGCCAACGAGCTCAATGAGATCG | |
| | ATGCCCAGCCCGGGCGCTACCACGTGCTGACCCTGCGGCAGCTGGCGCTCAAGGACTC | |
| | GGGCACCATCTACTTTGAGGCGGGTGACCAGCGGGCCTCGGCCGCCCTGCGGGTCACT | |
| | GAGAAGCCAAGCGTCTTCTCCCGGGAGCTCACAGATGCCACCATCACAGAGGGTGAGG | |
| | ACTTGACCCTGGTGTGCGAGACCAGCACCTGCGACATTCCTGTGTGCTGGACCAAGGA | |
| | TGGGAAGACCCTGCGGGGGTCTGCCCGGTGCCAGCTGAGCCATGAGGGCCACCGGGCC | |
| | CAGCTGCTCATCACTGGGGCCACCCTGCAGGACAGTGGACGCTACAAGTGTGAGGCTG | |
| | GGGGCGCCTGCAGCAGCTCCATTGTCAGGGTCCATGCGCGGCCAGTGCGGTTCCAGGA | |
| | GGCCCTGAAGGACCTGGAGGTGCTGGAGGGTGGTGCTGCCACACTGCGCTGTGTGCTG | |
| | TCATCTGTGGCTCCCCCCGTGAAGTGGTGCTATGGAAACAACGTCCTGAGGCCAGGTG | |
| | ACAAATACAGCCTACGCCAGGAGGGTGCCATGCTGGAGCTGGTCGTCCGGAACCTCCG | |
| | GCCGCAGGACAGCGGGCGGTACTCATGCTCCTTCGGCGACCAGACTACTTCTGCCACC | |
| | CTCACAGTGACTGCCCTCCCTGCCCAGTTCATCGGGAAACTGAGPAACAAGGAGGCCA | |
| | CAGAAGGGGCCACGGCCACGCTGCGGTGTGAGCTGAGCAAGGCAGCCCCTGTGGAGTG | |
| | GAGAAAGGGGTCCGAGACCCTCAGAGATGGGGACAGATACTGTCTGAGGCAGGACGGG | |
| | GCCATGTGTGAGCTGCAGATCCGTCGCCTGGCCATGGTGGATGCCGCGGAGTACTCGT | |
| | GTGTGTGTGGAGAGGAGAGGACCTCACCCTCACTCACCATCAGGCCCATGCCTGCCCA | |
| | CTTCATAGGAAGACTGAGACACCAAGAGAGCATAGAAGGGGCCACAGCCACGCTGCGG | |
| | TGTGAGCTGAGCAAGGCGGCCCCCGTGGAGTGGAGGAAGGGGCGTGAGAGCCTCAGAG | |
| | ATGGGGACAGACATAGCCTGAGGCAGGACGGGGCTGTGTGCGAGCTGCAGATCTGTGG | |
| | CCTGGCTGTGGCAGATGCTGGGGAGTACTCCTGTGTGTGTGGGGACGAGAGGACCTCT | |
| | GCCACTCTCACCGTGAAGGCCCTGCCAGCCAAGTTCACAGAGGGTCTGAGGAATGAAG | |
| | AGGCCGTGGAAGGGGCCACAGCCATGTTGTGGTGTGAACTGAGCAAGGTGGCCCCTGT | |
| | GGAGTGGAGGAAGGGGCCCGAGAACCTCAGAGATGGGGACAGATACATCCTGAGGCAG | |
| | GAGGGGACCAGGTGTGAGCTGCAGATCTGTGGCCTGGCCATGGCGGACGCCGGGGAGT | |
| | ACTTGTGTGTGTGCGGGCAGGAGAGGACCTCAGCCACGCTCACCATCAGGCCTCTGCC | |
| | TGCCAGGTTCATAGAAGATGTGAAAAACCAGGAGGCCAGAGAAGGGGCCACGGCTGTG | |
| | CTGCAGTGTGAGCTGAACAGTGCAGCCCCTGTGGAGTGGAGAAAGGGGTCTGAGACCC | |
| | TTAGACATGGGCACAGATACACCCTGAGGCAGGACGGGACTAAATGTGAGCTGCAGAT | |
| | TCGTGGCCTGGCCATGCCAGACACTGGGGAGTACTCGTGCGTGTGCGGGCAGGAGAGG | |
| | ACCTCGGCTATCCTCACCGTCAGGGCTCTACCCATCAAGTTCACAGAGGGTCTGAGGA | |
| | ACGAAGAGGCCACAGAAGGGGCAACAGCCGTGCTGCGGTGTGAGCTGAGCAAGATGCC | |
| | CCCCGTGGAGTGGTGGAAGGGGCATGAGACCCTCAGAGATGGAGACAGACACAGCCTG | |
| | AGGCAGGACGGGGCCAGGTGTGAGCTGCAGATCCGCGGCCTCGTGGCAGAGGACGCTG | |
| | GGGAGTACCTGTGCATGTGCCGGAAGGAGAGGACCTCAGCCATGCTCACCGTCAGGGC | |
| | CATGCCTTCCAAGTTCATAGAGGGTCTGAGGAATGAAGAGGCCACAGAAGGGGACACG | |
| | GCCACGCTGTGGTGTGAGCTGAGCAAGGCCGCACCGGTGGAGTGGAGGAAGGGGCATG | |
| | AGACCCTCAGAGATGGGGACAGACACAGCCTGAGGCAGGATGGGTCCAGGTGTGAGCT | |
| | GCAGATCCGTGGCCTGGCTGTGGTGGATGCCGGGGAGTACTCGTGTGTGTGCGGGCAG | |
| | CAGAGGACCTCAGCCACACTCACTGTCAGGGCCCTGCCTGCCAGATTCATAGAAGATG | |
| | TGAAAAACCAGGAGGCCAGAGAAGGGGCCACGGCCGTGCTGCAATGTGAGCTGAGCAA | |
| | GGCGGCCCCCGTGGAGTGGAGGAAGGGGTCTGAGACCCTCAGAGGTGGGCACAGATAC | |
| | AGCCTGACGCAGGATGGGACCAGATGTGAGCTGCAGATTCATGGCCTGTCTGTGGCAG | |
| | ACACTGGCGAGTACTCGTGTGTGTGCGGGCAGGAGAGGACCTCGGCCACACTCACCGT | |
| | CAGGGCCCTGCCTGCACGATTCACTCAAGATCTGAAGACCAAGGAGGCCTCAGAAGGG | |
| | GCCACAGCTACACTGCAGTGTGAGCTGAGCAAGGTGGCCCCTGTGGAATGGAAGAAGG | |
| | GTCCTGAGACCCTCAGAGATGGGGGCAGATACAGCCTGAAGCAGGATGGGACGAGGTG | |
| | TGAGCTGCAGATCCATGACCTGTCTGTGGCGGATGCTGGGGAATACTCATGCATGTGT | |
| | GGACAAGAGAGGACCTCGGCCACGCTCACTGTCAGGGCCCTGCCTGCCAGGTTCACAG | |
| | AGGGTCTGAGGAATGAACAGGCCATGGAAGGGGCCACAGCCACACTGCAATGTGAGCT | |
| | GAGCAAGGCAGCCCCTGTGGAGTGGAGGAAAGGCCTTGAGGCTCTCAGAGATGGGGAC | |
| | AAATACAGCCTGAGACAAGACGGGGCTGTGTGTGAGCTGCAGATTCATGGCCTGGCTA | |
| | TGGCAGATAACGGGGTGTACTCATGTGTGTGTGGGCACGAGAGCACCTCAGCTACACT | |
| | CACTGTCAGGGCCCTGCCTGCCAGATTCATAGAGGATATGAGAAACCAGAAGGCCACA | |
| | GAAGGGCCTACAGTCACATTGCAATGTAAGCTGAGAAAGGCGGCCCCCGTGGAGTCGA | |
| | GAAAGGGGCCCAACACCCTCAAAGATGGGGACAGGTACAGCCTGAAGCAGGATGGGAC | |
| | CAGTTGTGAGCTGCAGATTCGTGGCCTGGTCATAGCAGATGCTGGAGAATACTCGTGC | |
| | ATATGTGAGCAGGAGAGGACCTCGGCCACGCTCACTGTCAGGGCCCTGCCGGCCAGAT | |
| | TCATAGAAGATGTGAGAAATCACGAGGCCACAGAAGGGGCCACAGCTGTGCTGCAGTG | |
| | TGAGCTGAGCAAGGCGGCCCCCGTGGACTGGCGGAAGGGGTCTCAGACCCTCAGAGAT | |
| | GGGCACAGATATAGCCTGAGGCAGGACGCGACCAGGTGTGAGCTGCAGATTCGTGGCC | |
| | TGGCTGTGGAGGACACTGCAGAGTATTTGTGTGTGTGCGGGCAGGAGAGAACCTCAGC | |
| | TACACTCACTGTCAGGGCCCTGCCAGCCAGATTCATAGACAACATGACAAACCAGGAA | |
| | GCCAGAGAAGGCGCCACGGCCACACTGCACTGTGAACTGAGCAAGGTCGCCCCTGTGG | |
| | AGTGGAGGAAGGGACCTGAAACCCTCCGAGATGGGGACAGACACAGCCTGAGGCAGGA | |
| | TGGGTCCAGGTGTGAGCTGCAGATCCGTGGCCTGGCTGTGGTGGATGCCGGGGAGTAC | |
| | TCGTGTGTGTGCGGGCAGGAGAGGACCTCAGCCACACTCACTGTCAGGGCCCTGCCTG | |
| | CCAGATTCATAGAAGATGTGAAAAACCAGGAGGCCAGAGAAGGGGCCACGGCCGTGCT | |
| | GCAATGTGAGCTGAGCAAGGCGGCCCCCGTGGAGTGGAGGAAGGGGTCTGAGACCCTC | |
| | AGAGGTGGGGACAGATACAGCCTGAGGCAGGATGGGACCAGATGTGAGCTGCAGATTC | |
| | ATGGCCTGTCTGTGGCAGACACTGGGGAGTACTCGTGTGTGTGCGGGCAGGAGAGGAC | |
| | CTCGGCCACACTCACCGTCAGGGCCCTGCCTGCACGATTCACTCAAGATCTGAAGACC | |
| | AAGGAGGCCTCAGAAGGGGCCACAGCTACACTGCAGTGTGAGCTGAGCAAGGTGGCCC | |
| | CTGTGGAATGGAAGAAGGGTCCTGAGACCCTCAGAGATGGGGGCAGATACAGCCTGAA | |
| | GCAGGATGGGACGAGGTGTGAGCTGCAGATCCATGACCTGTCTGTGGCGGATGCTGGG | |
| | GAATACTCATGCATGTGTGGACAAGAGAGGACCTCGGCCACGCTCACTGTCAGGGACT | |
| | GCCACACTCTTCACGTCATGCCACACTATCCCTTCCAGCTTCCTGGGCTGCTGAAGGA | |
| | ACCAGAAGAAACTCTCATCTACATCCAGATTCCCTCTCCTGTGATACTGTTCACAGAG | |
| | GGTCTGAGGAATGAAGAGGCCATGGAAGGGGCCACAGCCACACTGCAATGTGAGCTGA | |
| | GCAAGGCAGCCCCTGTGGAGTGGAGGAAACCCCTTGAGGCTCTCAGAGATGGGGACAA | |
| | ATACAGCCTGAGACAAGACGGGGCTGTGTGTGAGCTGCAGATTCATGGCCTGGCTATG | |
| | GCAGATAACGGGGTGTACTCATCCCTGCCTGCCAGATTCATAGAGGATATGAGAAACC | |
| | AGAAGGCCACAGAAGGGGCTACAGTCACATTGCAATGTAAGCTGAGAAAGGCGGCCCC | |
| | CGTGCAGTCCACAAAGGGGCCCAACACCCTCAAAGATGGGGACAGGTACAGCCTGAAG | |
| | AATACTCGTGCATATGTGAGCAGGAGAGGACCTCGGCCACGCTCACTGTCAGGGCCCT | |
| | GCCGGCCAGATTCATAGAAGATGTGAGAAATCACGAGGCCACAGAAGGGGCCACAGCT | |
| | GTGCTGCAGTGTOACCTGAGCAAGGCGGCCCCCGTGGAGTGGCGGAAGGGGTCTGAGA | |
| | CCCTCAGAGATGGGGACAGATATAGCCTCAGGCAGGACGGGACGAGGTGTGAGCTGCA | |
| | GATTCGTGGCCTGGCTGTGGAGGACACTGGAGAGTATTTGTGTGTGTGCGGGCAGGAG | |
| | AGAACCTCAGCTACACTCACTGTCAGGGCCCTGCCAGCCAGATTCATAGACAACATGA | |
| | CAAACCAGGAAGCCACAGAACGGGCCACGCCCACACTGCACTGTGAACTGAGCAAGGT | |
| | GGCCCCTGTGGAGTGGAGGAAGGGACCTGAAACCCTCCGAGATGGGGACAGACACAGC | |
| | CTGAGGCAGGATGGGACCAGGTGTGAGCTGCAGATTCGTGGCCTGTCTGTGGCAGATG | |
| | CCGGGGAGTACTCGTGCGTGTGTGGGCAGGAGAGGACCTCAGCCACACTCACGATCAG | |
| | GGCCCTGCCCGCCAAGTTCACAAAGGGTCTGAGGAATGAAGAGGCCACAGAAGGGGCC | |
| | ACGGCTATGTTGCAGTGTCAGCTGAGCAAGGTGGCCCCTGTTGAGTGGAGGAAGGGAC | |
| | CTGAAACCCTCAGAGATGGGGACAGATACAACCTGAGGCAGGATGGGACCAGATGTGA | |
| | GCTGCAGATTCATGGCCTGTCCGTGGCAGACACTGGGGAGTACTCATGTGTATGTGGT | |
| | CAGGAGAAGACGTCGGCCACTCTCACTGTCAAGGCCCCACAGCCACTGTTCCGGGAGC | |
| | CGCTGCAGAGTCTGCAGGCGGAGGAGGGCTCCACGGCCACCCTGCAGTGTGACCTGTC | |
| | TGAGCCCACTGCTACAGTGGTCTGGAGCAAGGGTGGCCTGCAGCTGCAGGCCAATGGG | |
| | CGCCGGGAGCCACGGCTTCAGGGCTGCACCGCGGAGCTGGTGTTACAGGACCTACAAC | |
| | GTGAAGACACTGGCGAATACACTTGCACCTGTGGCTCCCAGGCCACCAGTGCCACCCT | |
| | CACTGTCACAGCTGCGCCTGTGCGGTTCCTCCGAGAGCTGCAGCACCAGGAGGTGGAT | |
| | GAGGGAGGCACCGCACACTTATGCTGCGAGCTGAGCCGGGCGGGTGCGAGCGTGGAGT | |
| | GGCCCAAGGGCTCCCTACAGCTCTTCCCTTGTGCCAAGTACCAGATGGTGCAGGATGG | |
| | TGCAGCTGCAGAGCTCCTGGTACGCGGAGTGGAGCAGGAGGATGCGGGTGACTACACG | |
| | TGTGACACGGGCCACACGCAGAGCATGGCCAGCCTCTCTGTCCGTGGAGGGCGTGGAG | |
| | CTGCATGCGGGCCCCAAGTACGAGATGCGGCGCAGGGGGCCACGCGGGAGCTGCTGAT | |
| | CCACCAACTGGAGGCCAAGGACACGGGCGAGTATGCCTGTGTGACAGGCGGCCAGAAA | |
| | ACCGCTGCCTCCCTCAGGGTCACAGAGCCTGAGGTGACCATTGTACGGGGGCTGGTTG | |
| | ATGCGCAGGTGACGGCCGATGAGCATGTTGAGTTCAGCTCTGAGGTGTCCAGGGCTGG | |
| | AGCCACAGGCGTGCAGTGGTGCCTACAGGGCCTGCCACTGCAAAGCAATGAGGTGACA | |
| | GAGGTGGCTGTGCGGGATGGCCGCATCCACACCCTGCCGCTGAAGGGCGTGACGCCCG | |
| | AGGACGCTGGCACTGTCTCCTTCCATTTGGGAAACCATGCTTCCTCTGCCCAGCTCAC | |
| | CGTCAGAGCTCCTGAGGTGACCATCCTGGAGCCCCTGCAGGACGTGCAGCTCAGAGGG | |
| | GTGCCCCTGCAGGCCAACGAGATGAATGACATCACTGTGGAGCAGGGCACACTCCACC | |
| | TGCTCACCCTGCACAAGGTGACCCTTGAGGATCCTGGAACTGTCAGTTTCCACGTGGG | |
| | CACGTGTAGCTCTGAGGCCCAGCTGAAAGTCACAGAGGCAGTGCCGTGCCTGGTACGT | |
| | GGCTTGCAGAATGTGGATGTCTTCGCGGGGGAGGTGGCCACGTTCTCCTGTGAGGATG | |
| | GCCCCCAGAGCGCCATCGCTGTGCGAGATGGGATCTTTCACTCCCTCATGCTCTCGGG | |
| | CCTGGGGGTGGCCGACTCCGCCACTGTCATCTTCCGCGCAGGGCCCCTGGTCTCCACG | |
| | GCCAAGTTGTTGATCAAAGATCCCGTCGTGGAGGTGGTCAGTGCCATGCAGGACTTGG | |
| | CCGTGGAGGAGGGTGGCTCGGCTGAGCTCCTCTGCCAGTATTCACGGCCCGTGCAGGC | |
| | CACGTGGAAGATGGACGAGCGGGAGGTGCACACGGATGGGCACCGTGTCATCATAGAG | |
| | CAGGACTGGAACGTGGCCAGGCTGACCTTCAGGCCGGCCTTGCCCTGTGACAGTGGCA | |
| | TCTATTCTTGTGAGGCTGCGGGCACCCGCGTAGTGGCCCTGCTGCAAGTGCAAGCCAA | |
| | GAACACGGTGGTGCGAGGGCTGGAGAATGTGGAGGCGCTGGAGGGCGGCGAGGCGCTG | |
| | TTCGAGTGCCAGCTGTCCCAGCCCGAGGTGGCCGCCCACACCTGGCTGCTGGACGACG | |
| | AACCCGTGCGCACCTCGGAGAACGCCGAGGTGGTCTTCTTCGAGAACGGCCTGCGCCA | |
| | CCTGCTGCTGCTCAAAAACTTGCGGCCACAAGACAGCTGCCGGGTGACCTTCCTGGCT | |
| | GGGGATATGGTGACGTCCGCATTCCTCACGGTCCGAGGTGACTGCGCTGTGCTGGTGC | |
| | AGGGCTGGCGCCTGGAGATCCTGGAGCCTCTGAAAAACGCGGCGGTCCGGGCCGGCGC | |
| | ACAGGCACGCTTCACCTGCACGCTCAGCGAGGCGGTGCCCGTGGGAGAGGCGTCCTGG | |
| | TACATCAATGGCGCGGCAGTGCAGCCGGATGACAGCGACTGGACTGTCACCGCCGACG | |
| | GCAGTCACCACGCCCTACTGCTGCGCAGCGCCCAGCCCCACCACGCCGGGGAGGTCAC | |
| | CTTCGCTTGCCGCGACGCCGTGGCCTCTGCGCGGCTCACCGTGCTGGGCCTCCCTGAT | |
| | CCCCCAGAGGATGCTGAGGTGGTGGCTCGCAGCAGCCACACTGTGACACTGTCTTGGG | |
| | CAGCTCCCATGAGTGATGGAGGCGGTGGTCTCTGTGGCTACCGCGTGGAGGTGAAGGA | |
| | GGGGGCCACAGGCCAGTGGCGGCTGTGCCACGAGCTGGTGCCTGGACCCGAGTGTGTG | |
| | GTGGATGGCCTGGCCCCCGGGGAGACCTACCGCTTCCGTGTGGCAGCTGTGGGCCCTG | |
| | TGGGTGCTGGGGAACCGGTTCACCTGCCCCAGACAGTGCGGCTTGAGCCACCGAAGCC | |
| | TGTGCCTCCCCAGCCCTCAGCCCCTGAGAGCCGGCAGGTGGCAGCTGGTGAAGATGTC | |
| | TGTCTGCAGCTTGAGGTCGTGGCTGAGGCTGGCGAGGTCATCTGGCACAAGGGAATGG | |
| | AGCGCATCCAGCCCGGTGGGCGGTTCGAGGTGGTCTCCCAGGGTCGGCAACAGATGCT | |
| | GGTGATCAAGGGCTTCACGGCAGAAGACCAGGGCGAGTACCACTGTGGCCTGGCTCAG | |
| | GGCTCCATCTGCCCTGCGGCTGCCACCTTCCAGGTGGCACTCAGCCCAGCCTCTGTGG | |
| | ATGAGGCCCCTCAGCCCAGCTTGCCCCCCGAGGCAGCCCAGGAGGGTGACCTGCACCT | |
| | ACTGTGGGAGGCCCTGGCTCGGAAACGTCGCATGAGCCGTGAGCCCACGCTGGACTCC | |
| | ATTAGCGAGCTGCCAGAGGAGGACGGCCGCTCGCAGCGCCTGCCACAGGAGGCAGAGG | |
| | AGGTGGCACCTGATCTCTCTGAAGGCTACTCCACGGCCGATGAGCTGGCCCGCACTGG | |
| | AGATGCTGACCTCTCACACACCAGCTCTGATGATGAGTCCCGGGCAGGCACCCCTTCC | |
| | CTGGTCACCTACCTCAAGAAGGCTGGGAGGCCAGGCACCTCACCACTGGCCAGCAAGG | |
| | TGAGCCCCCCCAACTTGGCCTGCAAGGAGAGGTTCCCCACGCCCCGGGCCGGCCGCAG | |
| | CCTCCTGGGCTTCGTGGGGGCAGACCCAGCCTTTCCCGGCAGCGAGCGCTCGGCCAGG | |
| | TGCACTAGGCGCTGTGCGGCCCCCCCTCCCCGCGAGTCCCTCAAGCGGGAACCTGCCT | |
| | CGTGTCTCCCAGGAGCCATGGAGGCTGTGGAACTCGCCAGAAAACTGCAGGAGGAAGC | |
| | TACGTGCTCCATCTGTCTGGATTACTTCACAGACCCTGTGATGACCACCTGTGGCCAC | |
| | AACTTCTGCCGAGCGTGCATCCAGCTGAGCTGGGAAAAGGCGAGGGGCAAGAAGGGGA | |
| | GGCGGAAGCGGAAGGGCTCCTTCCCCTGCCCCGAGTGCAGAGACATGTCCCCCCACAG | |
| | GAACCTGCTGCCCAACCGGCTGCTGACCAAGGTGGCCGAGATGGCGCAGCAGCATCCT | |
| | CGTCTGCAGAAGCAAGACCTGTGCCAGGAGCACCACGAGCCCCTCAAGCTTTTCTGCC | |
| | AGAAGGACCAGAGCCCCATCTGTGTGGTGTGCAGGGAGTCCCGGGAGCACCGGCTGCA | |
| | CAGGGTGCTGCCCGCCGAGGAGGCAGTGCAGGGGTACAAGTTGAACCTGGAGGAGGAC | |
| | ATGGAGTACCTTCGGGAGCAGATCACCAGGACAGGGAATCTGCAGGCCAGGGAGGAGC | |
| | AGAGCTTAGCCGAGTGGCAGGGCAAGGTGAAGGAGCGGAGAGAACGCATTGTGCTGGA | |
| | GTTTGAGAAGATGAACCTCTACCTGGTGGAAGAAGAGCAGAGGCTCCTCCAGGCTCTG | |
| | GAGACCGAAGAAGAGGAGACTCCCACCAGGCTCCGGGAGAGCGTGGCCTGCCTGGACC | |
| | GGCAGGGTCACTCTCTGGAGCTGCTGCTGCTGCAGCTGCAGGAGCCGAGCACACAGGG | |
| | GCCCCTCCAGATGCTGCAGGACATGAAGCAACCCCTGACCAGGGCGGCGTTACTGGTG | |
| | GTTCTAATTCATGGGATGAATCTTGTTGAGTTCCCAGTGGTCTCTCTGCCCAGCCCCC | |
| | TGTACCTTATTGCCACCAAGGCCCACACACAATTGGGCCCGGGGACTCCCACCTTTGA | |
| | CCCTGAATGCCCCACACCTCTCCCCATCTCTCCACCACCACGCCCATCTACAGAGGAT | |
| | GTGGTGCCTGATGCCACCTCCGCGTACCCCTACCTCCTCCTGTATGAGAGCCGCCAGA | |
| | GGCGCTACCTCGGCTCTTCGCCGGAGGGCAGTGGGTTCTGCAGCAAGGACCGATTTGT | |
| | GGCTTACCCCTGTGCTGTGCGCCAGACGGCCTTCTCCTCTGGGAGGCACTACTGGGAG | |
| | GTGGGCATGAACATCACCGGGGACGCGTTGTGGGCCCTGGGTGTGTGCAGGGACAACG | |
| | TGAGCCGGAAAGACAGGGTCCCCAAGTGCCCCGAAAACGGCTTCTGGGTGGTGCAGCT | |
| | GTCCAAGGGGACCAAGTACTTATCCACCTTCTCTGCCCTAACCCCGGTCATGCTGATG | |
| | GAGCCTCCCAGCCACATGGGCATCTTCCTGGACTTCGAAGCCCGGGAAGTGTCCTTCT | |
| | ACAGTGTAAGCGATGGGTCCCACCTGCACACCTACTCCCAGGCCACCTTCCCAGGCCC | |
| | CCTGCAGCCTTTCTTCTGCCTGGGGGCTCCGAAGTCTGGTCAGATGGTCATCTCCACA | |
| | GTGACCATGGCAGGGGTAAAAGACCTGGCCACAAGAACCGGAGCGGTGGTGACGCCAG | |
| | CGCTCGGAGCCTACGCGCCCAGCGCTACCGAAACCCAGAGTCCTGCGCCCTGGAGTCC | |
| | CCGCGCCCCGGAGCCCGAGCACCCGGGAGTCCCGAGCCTCGCGCCCCGGAGTGCCCGA | |
| | GCCTGCGCCGCCGCACCCGGATACCCCGGGTCCCCGCGAGCTGCCGAGGCCGCCCGCC | |
| | GCCGCCCCGCGGACAGTACCGCCTTCCTCCCCTCTGTCCGCGCCATGGCCGCCCCCGA | |
| | CCTGTCCACCAACCTCCAGGAGCAGGCCACCTGCGCCATCTGCCTCGACTACTTCACG | |
| | GATCCGGTGATGACCGACTGCGGCCACAACTTCTGCCGCGAGTGCATCCGGCGCTGCT | |
| | GGGGCCAGCCCGAGGCCCGTACGCGTGCCCCGAGTGCCGCGAGCTGTCCCCGCAGAGG | |
| | AACCTGCGGCCCAACCGCCCGCTTGCTAAGATGGCCGAGATGGCGCGGCGCCTGCACC | |
| | CGCCGTCGCCGGTCCCGCAGGCGTGTGCCCGCGCACCGCGAGCCACTGGCCGCCTTCT | |
| | GTGGCGACGAGCTGCGCCTCCTGTGTGCGGCCTGCGAGCGCTCTGGGGAGCACTGGGC | |
| | GCACCGCGTTGGCCGCTGCAGGACGCGGCCGAAGACCTCAAGGCCCCTTGAGGCTGGG | |
| | ACCATGGCCGCCAATGAGACCCTGCTCTCGGGGGCGAAGCTGGAGAAGTCACTGGAGC | |
| | ATCTCCGGAAGCAGATGCAGGATGCGTTGCTGTTCCAAGCCCAGGCGGATGAGACCTG | |
| | CGTCTTGTGGCAGGCAGAAGATGGTGGAGAGCAGCGGCACAACCTGCTGCCTGAGTTC | |
| | GAGCGTCTTCGCCGTTTGCTGGCAGAGGGAGGGACAGCAGCTGCTGCAGAGGCTGGAG | |
| | AGGAGGAGCTGAAGCAGAGCGCCCACCTAGCTGAGCTCATCGCCGAGCTCGAGAGGCC | |
| | GCTGCCAGCTGCCTGCGCTGGGGCTGCTGCAGGAGAGTCTTTTCCCATGTGTGGGCTC | |
| | CACTCCCTGAGCCGGCCCCCTGGCGTGGGCTTTCCTTGGTGCACCCCCAAACCAGAAC | |
| | CAGTGGACGCCCTGGCCTGTGCGTGGCGGCAGGGCTGCCAGACCCAGGTGGAGCCCAC | |
| | AATGCTGCAGATGTGGCTGGGCGGCTTTGCACAGGGGGTGACACTGCTGCCGGCCTCT | |
| | GGAGCCCAGCAGAACATCAGTCCAGGCACCGGCTCCTGGTTTCGATTGTCATTTCTAT | |
| | TATTTAAGGGGTACAAGTGCAGTCAGAGTGTAGCCATCACCCGAATGGTGCACACTGT | |
| | ACCCAAGACCAAACCCCCTTGTCGAGGCCAAGGTTCTCCTCTACCCCCAAGCCCTTCT | |
| | CCTGCCGCCCCTGCACCCGGCCTTGTGACAGCCACCACCTGTTTCCAAATGACACCAG | |
| | GGGTGGGCCGCCCACCCCAGGACATCAAGGACGCCCTGCGCAGGGTCCAGGATGTGAA | |
| | GCTGCAGCCCCCAGAAGTTGTGCCTATGGAGCTGAGGACCGTGTGCAGGGTCCCGGGA | |
| | CTGGTAGAGACACTGCGCAGGTTTCGAGGGGACGTGACCTTGGACCCGGACACCGCCA | |
| | ACCCTGACCTGATCCTGTCTGAAGACAGGCGGAGCGTGCAGCGGGGGGACCTACGGCA | |
| | GGCCCTGCCGGACAGCCCAGAGCGCTTTGACCCCGGCCCCTGCGTGCTGGCCCAGGAG | |
| | CGCTTCACCTCAGGCCGCCACTACTGGGAGGTGGAGGTTGGGGACCGCACCAGCTGGG | |
| | CCCTGGGGGTGTGCAGGGAGAACGTGAACAGGAAGGAGAAGGGCGAGCTGTCCGCGGG | |
| | CAACGGCTTCTGGATCCTGGTCTTCCTGGGGAGCTATTACAATTCCTCGGAACGGGCC | |
| | TTGGCTCCACTCCGGGACCCACCCAGGCGCGTGGGGATCTTTCTGGACTACGAGGCTG | |
| | GACATCTCTCTTTCTACAGTGCCACCGATGGGTCACTGCTATTCATCTTTCCCGAGAT | |
| | CCCCTTCTCGGGGACCCTGCGGCCCCTCTTCTCACCCCTGTCCAGCAGCCCGACCCCG | |
| | ATGACTATCTGCCGGCCGAAAGGTGGGTCCGOGGACACCCTGGCTCCCCAGTGACTCG | |
| | GGCCCTCCTGGAGGA | |
| | ORF Start: ATG at 15 | ORF Stop: TGA at 14088 |
| | SEQ ID NO:72 | 4691 aa MW at 512894.2 kD |
| NOV13a, | MPLYNDSFHEISHKGRRHTLVLKSIQRADAGIVRASSLKVSTSARLEVRVKPVVFLKA | |
| CG59237-01 Protein | LDDLSAEERGTLALQCEVSDPEAHVVWRKDGVQLGPSDKYDFLHTAGTRGLVVHDVSP | |
| Sequence | EDAGLYTCHVGSEETRARVRVHDLHVGITKRLKTMEVLEGESCSFECVLSHESASDPA | |
| | MWTVGGKTVGSSSRFQATRQGRKYILVVREAAPSDAGEVVFSVRGLTSKASLIVRERP | |
| | AAIIKPLEDQWVAPGEDVELRCELSRAGTPVHWLKDRKAIRKSQKYDVVCEGTMAMLV | |
| | IRGASLKDAGEYTCEVEASKSTASLHVEEKANCFTEELTNLQVEEKGTAVFTCKTEHP | |
| | AATVTWRKGLLELRASGKHQPSQEGLTLRLTISALEKADSDTYTCDIGQAQSRAQLLV | |
| | QGRRVHIIEDLEDVDVQEGSSATFRCRISPANYEPVHWFLDKTPLHANELNEIDAQPG | |
| | GYHVLTLRQLALKDSGTIYFEAGDQRASAALRVTEKPSVFSRELTDATITEGEDLTLV | |
| | CETSTCDIPVCWTKDGKTLRGSARCQLSHEGHRAQLLITGATLQDSGRYKCEAGGACS | |
| | SSIVRVHARPVRFQEALKDLEVLEGGAATLRCVLSSVAAPVKWCYGNNVLRPGDKYSL | |
| | RQEGANLELVVRNLRPQDSCRYSCSFGDQTTSATLTVTALPAQFIGKLRNKEATEGAT | |
| | ATLRCELSKAAPVEWRKGSETLRDGDRYCLRQDGAMCELQIRGLAMVDAAEYSCVCCE | |
| | ERTSASLTIRPMPAHFIGRLRHQESIEGATATLRCELSKAAPVEWRKGRESLRDGDRH | |
| | SLRQDGAVCELQICGLAVADAGEYSCVCGEERTSATLTVKALPAKFTEGLRNEEAVEG | |
| | ATANLWCELSKVAPVEWRKGPENLRDGDRYILRQEGTRCELQICGLAMADAGEYLCVC | |
| | GQERTSATLTIRALPARFIEDVKNQEAREGATAVLQCELNSAAPVEWRKGSETLRDGD | |
| | RYSLRQDGTKCELQIRGLAMADTGEYSCVCGQERTSANLTVRALPIKFTEGLRNEEAT | |
| | EGATAVLRCELSKMAPVEWWKGHETLRDGDRHSLRQDGARCELQIRGLVAEDAGEYLC | |
| | MCGKERTSAMLTVRAMPSKFIEGLRNEEATEGDTATLWCELSKAAPVEWRKGHETLRD | |
| | GDRHSLRQDGSRCELQIRGLAVVDAGEYSCVCGQERTSATLTVRALPARFIEDVKNQE | |
| | AREGATAVLQCELSKAAPVEWRKGSETLRGGDRYSLRQDGTRCELQIHGLSVADTGEY | |
| | SCVCGQERTSATLTVRALPARFTQDLKTKEASEGATATLQCELSKVAPVEWKKGPETL | |
| | RDGGRYSLKQDGTRCELQIHDLSVADAGEYSCMCGQERTSATLTVRALPARFTEGLRM | |
| | EEANEGATATLQCELSKAAPVEWRKGLEALRDGDKYSLRQDGAVCELQIHGLAMADNG | |
| | VYSCVCGQERTSATLTVRALPARFIEDMRNQKATEGATVTLQCKLRKAAPVEWRKGPN | |
| | TLKDGDRYSLKQDGTSCELQIRGLVIADAGEYSCICEQERTSATLTVRALPARFIEDV | |
| | RNHEATEGATAVLQCELSKAAPVEWRKGSETLRDCDRYSLRQDGTRCELQIRGLAVED | |
| | TGEYLCVCGQERTSATLTVRALPARFIDNMTNQEAREGATATLHCELSKVAPVEWRKG | |
| | PETLRDGDRHSLRQDGSRCELQIRGLAVVDAGEYSCVCGQERTSATLTVRALPARFIE | |
| | DVKNQEAREGATAVLQCELSKAAPVEWRKGSETLRGGDRYSLRQDGTRCELQIHGLSV | |
| | ADTGEYSCVCGQERTSATLTVRALPARFTQDLKTKEASEGATATLQCELSKVAPVEWK | |
| | KGPETLRDGGRYSLKQDGTRCELQIHDLSVADAGEYSCMCGQERTSATLTVRDCHTLH | |
| | VMPHYPFQLPGLLKEPEETLIYIQIPSPVILFTEGLRNEEANEGATATLQCELSKAAP | |
| | VEWRKGLEALRDGDKYSLRQDGAVCELQIHGLAMADNGVYSSLPARFIEDMRNQKATE | |
| | GATVTLQCKLRKAAPVEWRKGPNTLKDGDRYSLKQDGTSCELQIRGLVIADAGEYSCI | |
| | CEQERTSATLTVRALPARFIEDVRNHEATEGATAVLQCELSKAAPVEWRKGSETLRDG | |
| | DRYSLRQDGTRCELQIRGLAVEDTGEYLCVCGQERTSATLTVRALPARFIDNNTNQEA | |
| | REGATATLHCELSKVAPVEWRKGPETLRDGDRHSLRQDGTRCELQIRGLSVADAGEYS | |
| | CVCGQERTSATLTIRALPAKFTKGLRNEEATEGATAMLQCELSKVAPVEWRKGPETLR | |
| | DGDRYNLRQDGTRCELQIHGLSVADTGEYSCVCGQEKTSATLTVKAPQPVFREPLQSL | |
| | QAEEGSTATLQCELSEPTATVVWSKGGLQLQANGRREPRLQGCTAELVLQDLQREDTG | |
| | EYTCTCGSQATSATLTVTAAPVRFLRELQHQEVDEGGTARLCCELSRAGASVEWRKGS | |
| | LQLFPCAKYQMVQDGAAAELLVRGVEQEDAGDYTCDTGHTQSMASLSVRGGRGARCGP | |
| | QVRDAAQGATRELLIHQLEAKDTGEYACVTGGQKTAASLRVTEPEVTIVRGLVDAEVT | |
| | ADEDVEFSCEVSRAGATGVQWCLQGLPLQSNEVTEVAVRDGRIHTLRLKGVTPEDAGT | |
| | VSFHLGNHASSAQLTVRAPEVTILEPLQDVQLRGVPLQANEMNDITVEQGTLHLLTLH | |
| | KVTLEDAGTVSFHVGTCSSEAQLKVTEAVPCLVRGLQNVDVFAGEVATFSCEDGPQSA | |
| | IAVRDGIFHSLMLSGLGVADSGTVIFRAGPLVSTAKLLIKDPVVEVVSAMQDLAVEEG | |
| | GSAELLCQYSRPVQATWKMDEREVHTDGHRVIIEQDWNVARLTFRPALPCDSGIYSCE | |
| | AAGTRVVALLQVQAKNTVVRGLENVEALEGGEALFECQLSQPEVAAHTWLLDDEPVRT | |
| | SENAEVVFFENGLRHLLLLKNLRPQDSCRVTFLAGDMVTSAFLTVRGDCAVLVQGWRL | |
| | EILEPLKNAAVRAGAQARFTCTLSEAVPVGEASWYINGAIWQPDDSDWTVTADGSHHA | |
| | LLLRSAQPHHAGEVTFACRDAVASARLTVLGLPDPPEDARVVARSSHTVTLSWAAPMS | |
| | DGGCGLCGYRVEVKEGATGQWRLCHELVPGPECVVDGLAPGETYRFRVAAVGPVGAGE | |
| | PVHLPQTVRLEPPKPVPPQPSAPESRQVAAGEDVCLELEVVAEAGEVIWHKGMERIQP | |
| | GGRFEVVSQGRQQMLVIKGFTAEDQGEYHCGLAQGSICPAAATFQVALSPASVDEAPQ | |
| | PSLPPEAAQEGDLHLLWEALARKRRMSREPTLDSISELPEEDGRSQRLPQEAEEVAPD | |
| | LSEGYSTADELARTGDADLSHTSSDDESRAGTPSLVTYLKKAGRPGTSPLASKVSPPN | |
| | LACKERFPTPRAGRSLLGFVCADPAFPGSERSARCTRRCAAPPPRESLKREPASCLPG | |
| | AMEAVELARKLQEEATCSICLDYFTDPVMTTCGHNFCRACIQLSWEKARGKKGRRKRK | |
| | GSFPCPECREMSPQRNLLPNRLLTKVAEMAQQHPGLQKQDLCQEHHEPLKLFCQKDQS | |
| | PICVVCRESREHRLHRVLPAEEAVQGYKLKLEEDMEYLREQITRTGNLQAREEQSLAE | |
| | WQGKVKERRERIVLEFEKMNLYLVEEEQRLLQALETEEEETASRLRESVACLDRQGHS | |
| | LELLLLQLEERSTQGPLQMLQDMKEPLSPAALLVVLIHGMNLVEFPVVSLPSPLYLIA | |
| | TKAHTQLGPGTPTFDPECPTPLPISPPPRPSTEDVVPDATSAYPYLLLYESRQRRYLG | |
| | SSPEGSGFCSKDRFVAYPCAVGQTAFSSGRHYWEVGMNITGDALWALGVCRDNVSRKD | |
| | RVPKCPENGFWVVQLSKGTKYLSTFSALTPVMLMEPPSHMGIFLDFEAGEVSFYSVSD | |
| | GSHLHTYSQATFPGPLQPFFCLGAPKSGQMVISTVTMAGVKDLATRTGAVVTPALGAY | |
| | APSATETQSPAPWSPRAPEPEHPGVPSLAPRSARACAAAPGYPGSPRAAEAARRRPAD | |
| | STAFLPSVRAMAAPDLSTNLQEEATCAICLDYFTDPVMTDCGHNFCRECIRRCWGQPE | |
| | ARTRAPSAASCPRRGTCGPTARLLRWPRWRGACTRRRRSRRRVPAHREPLAAFCGDEL | |
| | RLLCAACERSGEHWAHRVGRCRTRPKTSRPLEAGTMAANETLLSGAKLEKSLEHLRKQ | |
| | MQDALLFQAQADETCVLWQAEDGGEQRQNVLREFERLRRLLAEGGTAAAAEAGEEELK | |
| | QSAHLAELIAELERPLPAACAGAAAGESFPMCGLHSLSRPPGVGFPWCTPKPEPVDAL | |
| | ACAWRQGCQTQVEPTMLQMWLGGFAQGVTLLPASGAQQNISPGTGSWFRLSFLLFKGY | |
| | KCSQSVAITRMVHTVPKTKPPCRGQGSPLPPSPSPAAPAPGLVTATTCFQMTPGVGRP | |
| | PQDIKDALRRVQDVKLQPPEVVPMELRTVCRVPGLVETLRRFRGDVTLDPDTANPELI | |
| | LSEDRRSVQRGDLRQALPDSPERFDPGPCVLGQERFTSCRHYWEVEVGDRTSWALGVC | |
| | RENVNRKEKGELSAGNGFWILVFLGSYYNSSERALAPLRDPPRRVGIFLDYEAGHLSF | |
| | YSATDGSLLFIFPEIPFSGTLRPLFSPLSSSPTPMTICRPKGGSGDTLAPQ | |
| | SEQ ID NO:73 | 14061 bp |
| NOV13b, | TGTCGCTCAACGGGATGCCCCTGTACAACGACAGCTTCCATGAGATCTCACACAAGGG | |
| CG59237-02 DNA | CCGGCGCCACACGCTGGTACTGAAGAGCATCCAGCGGGCTGATGCGCGCATAGTACGC | |
| Sequence | GCCTCCTCCCTGAAGGTGTCGACCTCTGCCCGCCTGGAGGTCCGAGTGAAGCCGGTGG | |
| | TGTTCCTGAAGGCGCTGGATGACCTGTCCGCAGAGGAGCGCGGCACCCTGGCCCTGCA | |
| | GTGTGAAGTCTCTGACCCCGAGGCCCATGTGGTGTGGCGCAAAGATGGCGTGCAGCTG | |
| | GGCCCCAGTGACAAGTATGACTTCCTGCACACGGCGGGCACGCGGGGGCTCGTGGTGC | |
| | ATGACGTGAGCCCTGAAGACGCCGGCCTGTACACCTGCCACGTGGGCTCCGAGGAGAC | |
| | CCGGGCCCGCGTCCGCCTCCACGATCTGCACGTGGGCATCACCAAGAGGCTGAAGACA | |
| | ATGGAGGTGCTGGAAGGGGAAAGCTGCAGCTTTGAGTGCGTCCTGTCCCACGAGAGTG | |
| | CCAGCGACCCGGCCATGTGGACAGTCGGTGGGAAGACAGTGGGCAGCTCCAGCCGCTT | |
| | CCAGGCCACACGTCAGGGCCGAAAATACATCCTGGTGGTCCGGGAGGCTGCACCAAGT | |
| | GATGCCGGGGAGGTGGTCTTCTCTGTGCGGGGCCTCACCTCCAAGGCCTCACTCATTG | |
| | TCAGAGAGAGGCCGGCCGCCATCATCAAGCCCCTGGAAGACCAGTGGGTGGCGCCAGG | |
| | GGAGGACGTGGAGCTGCCCTGTGAGCTGTCACGGGCGGGAACGCCCGTGCACTGGCTG | |
| | AAGGACAGGAAGGCCATCCGCAAGAGCCAGAAGTATGATGTGGTCTGCGAGGGCACGA | |
| | TGGCCATGCTGGTCATCCGCGGGGCCTCGCTCAAGGACGCGGGCGAGTACACGTGTGA | |
| | GGTGGAGGCTTCCAAGAGCACAGCCAGCCTCCATGTGGAAGAAAAAGCAAACTGCTTC | |
| | ACAGAGGAGCTGACCAATCTGCAGGTGGAGGAGAAAGGCACAGCTGTGTTCACGTGCA | |
| | AGACGGAGCACCCCGCGGCCACAGTGACCTGGCGCAAGGGCCTCTTGGAGCTACGGGC | |
| | CTCAGGGAAGCACCAGCCCAGCCAGGAGGGCCTGACCCTGCGCCTCACCATCAGTGCC | |
| | CTGGAGAAGGCAGACAGCGACACCTATACCTGCGACATTGGCCAGGCCCAGTCCCGGG | |
| | CCCAGCTCCTAGTGCAAGGCCGGAGAGTGCACATCATCGAGGACCTGGACGATGTGGA | |
| | TGTGCAGGAGGGCTCCTCGGCCACCTTCCGTTGCCGGATCTCCCCGGCCAACTACGAG | |
| | CCTGTGCACTGGTTCCTGGACAAGACACCCCTGCATGCCAACGAGCTCAATGAGATCG | |
| | ATGCCCAGCCCGGGGGCTACCACGTGCTGACCCTGCGCCAGCTGGCGCTCAAGGACTC | |
| | GGGCACCATCTACTTTGAGGCGGGTGACCAGCGGGCCTCGGCCGCCCTGCGGGTCACT | |
| | CAGAAGCCAAGCGTCTTCTCCCGGGAGCTCACAGATGCCACCATCACAGAGGGTGAGG | |
| | ACTTGACCCTGGTGTGCGAGACCAGCACCTGCGACATTCCTGTGTGCTGGACCAAGGA | |
| | TGGCAAGACCCTGCGGGGGTCTGCCCGGTGCCAGCTGAGCCATGAGGGCCACCGGGCC | |
| | CAGCTGCTCATCACTGGGGCCACCCTGCAGGACAGTGGACGCTACAAGTGTGAGGCTG | |
| | GGGGCGCCTGCAGCACCTCCATTGTCAGCGTGCATGCGCGGCCAGTGCGGTTCCAGGA | |
| | GGCCCTGAAGGACCTGGAGGTGCTGGAGGCTGGTGCTGCCACACTCCGCTGTGTGCTG | |
| | TCATCTGTGGCTGCGCCCGTGAAGTGGTGCTATGGAAACAACGTCCTGAGGCCAGGTG | |
| | ACAAATACAGCCTACGCCAGGAGGGTGCCATGCTGGAGCTGGTGGTCCGGAACCTCCG | |
| | GCCGCAGGACAGCGGGCGGTACTCATGCTCCTTCGGGGACCAGACTACTTCTGCCACC | |
| | CTCACAGTGACTGCCCTGCCTGCCCAGTTCATCGGGAAACTGAGAAACAAGGAGGCCA | |
| | CAGAAGGGGCCACGGCCACGCTGCGGTGTGAGCTGAGCAAGGCAGCCCCTGTGGAGTG | |
| | GAGAAAGGGGTCCGAGACCCTCAGAGATGGGGACAGATACTGTCTGAGGCATTACGGG | |
| | GCCATGTGTGAGCTGCAGATCCGTGGCCTGGCCATGGTGGATGCCGCGGAGTACTCGT | |
| | GTGTGTGTGGAGAGGAGAGGACCTCAGCCTCACTCACCATCAGGCCCATGCCTGCCCA | |
| | CTTCATAGGAAGACTGAGACACCAAGAGAGCATAGAACGGGCCACAGCCACGCTGCGG | |
| | TGTGAGCTGAGCAAGGCGGCCCCCGTGGAGTGGAGGAAGGGGCGTGAGAGCCTCAGAG | |
| | ATGGGCACAGACATAGCCTGAGGCAGGACGGGGCTGTGTGCGAGCTGCAGATCTGTGG | |
| | CCTGGCTGTGGCAGATGCTGGGGAGTACTCCTCTGTGTGTGGGGAGGAGAGGACCTCT | |
| | GCCACTCTCACCGTGAAGGCCCTGCCAGCCAAGTTCACAGAGCCTCTGAGGAATGAAG | |
| | ACGCCGTGGAAGGGGCCACAGCCATGTTGTGGTGTGAACTGAGCAAGGTGGCCCCTGT | |
| | GGAGTGGAGGAAGGGGCCCGAGAACCTCAGAGATGGGGACAGATACATCCTGAGGCAG | |
| | GAGGGGACCAGGTGTGAGCTGCAGATCTGTGGCCTGGCCATGGCGGACGCCGGGOAGT | |
| | ACTTGTGTGTGTGCGGGCAGGAGAGGACCTCAGCCACGCTCACCATCAGGGCTCTGCC | |
| | TGCCAGGTTCATAGAAGATGTGAAAAACCAGGAGGCCAGAGAAGGGGCCACGGCTGTG | |
| | CTGCAGTGTGAGCTGAACAGTGCAGCCCCTGTGGAGTGGAGAAAGGGGTCTGAGACCC | |
| | TTAGAGATGCGGACAGATACAGCCTGAGGCAGGACGGGACTAAATGTGAGCTGCAGAT | |
| | TCGTGGCCTGGCCATCGCAGACACTGGGGAGTACTCGTGCGTGTGCGGGCAGGAGAGG | |
| | ACCTCGGCTATGCTCACCGTCAGGGCTCTACCCATCAAGTTCACAGAGGGTCTGAGGA | |
| | ACGAAGAGGCCACAGAAGGGGCAACAGCCGTGCTGCGGTGTGACCTGAGCAAGATGGC | |
| | CCCCGTGGAGTGGTGGAAGGGGCATGAGACCCTCAGAGATGGAGACACACACAGCCTC | |
| | AGGCAGGACGGGGCCAGGTGTGAGCTGCAGATCCGCGGCCTCGTGGCAGAGGACGCTG | |
| | GGGAGTACCTGTGCATGTGCGGGAAGGAGAGGACCTCAGCCATGCTCACCGTCAGGGC | |
| | CATGCCTTCCAAGTTCATAGAGGGTCTGAGGAATGAAGAGGCCACAGAAGGGGACACG | |
| | GCCACGCTGTGGTGTGAGCTGAGCAAGGCGGCACCGGTGGAGTGGAGGAAGGGGCATG | |
| | AGACCCTCAGAGATGGGGACAGACACAGCCTGAGGCAGGATGGGTCCAGGTGTGAGCT | |
| | GCAGATCCGTGGCCTGGCTGTGGTGGATGCCGGGGAGTACTCGTGTGTGTGCGCCCAG | |
| | GAGAGGACCTCAGCCACACTCACTGTCAGGGCCCTGCCTGCCAGATTCATAGAAGATG | |
| | TGAAAAACCAGGAGGCCAGAGAAGGGGCCACGGCCGTGCTGCAATGTGAGCTGAGCAA | |
| | GGCGGCCCCCGTGGAGTGGAGGAAGGGGTCTGAGACCCTCAGAGGTGGGGACAGATAC | |
| | AGCCTGAGGCAGGATGGGACCAGATGTGAGCTGCAGATTCATGGCCTGTCTGTGGCAG | |
| | ACACTGGGGAGTACTCGTGTGTGTGCGGGCAGGAGAGGACCTCGGCCACACTCACCGT | |
| | CAGGGCCCTGCCTGCACGATTCACTCAAGATCTGAAGACCAAGGAGGCCTCAGAAGGG | |
| | GCCACAGCTACACTGCAGTGTGAGCTGAGCAAGGTGGCCCCTGTGGAATGGAAGAAGG | |
| | GTCCTGAGACCCTCAGAGATGGGGGCAGATACAGCCTGAAGCAGGATGGGACGAGGTG | |
| | TGAGCTGCAGATCCATGACCTGTCTGTGGCGGATGCTGGGGAATACTCATGCATGTGT | |
| | GGACAAGAGAGGACCTCGGCCACGCTCACTGTCAGGGCCCTGCCTGCCAGGTTCACAG | |
| | AGGGTCTGAGGAATGAAGAGGCCATGGAAGGGGCCACAGCCACACTGCAATGTGAGCT | |
| | GAGCAAGGCAGCCCCTGTGGAGTGGAGGAAAGGCCTTGAGGCTCTCAGAGATGCGGAC | |
| | AAATACAGCCTGAGACAAGACGGGGCTGTGTGTGAGCTGCAGATTCATGGCCTGGCTA | |
| | TGGCAGATAACGGGGTGTACTCATGTGTGTGTGGGCAGGAGAGGACCTCAGCTACACT | |
| | CACTGTCAGGGCCCTGCCTGCCAGATTCATAGAGGATATGAGAAACCAGAAGGCCACA | |
| | GAAGGGGCTACAGTCACATTGCAATGTAAGCTGAGAAAGGCGGCCCCCGTGGAGTGGA | |
| | GAAAGGGGCCCAACACCCTCAAAGATGGGGACAGGTACAGCCTGAACCAGGATGGGAC | |
| | CAGTTGTGAGCTGCAGATTCGTGGCCTGGTCATAGCAGATGCTGGAGAATACTCGTGC | |
| | ATATGTGAGCAGGAGAGCACCTCGGCCACGCTCACIGTCAGGGCCCTGCCGGCCAGAT | |
| | TCATAGAAGATGTGAGAAATCACGAGGCCACAGAAGGGGCCACAGCTGTGCTCCAGTG | |
| | TGAGCTGAGCAAGGCGGCCCCCGTGGAGTGGCGGAAGGGGTCTGAGACCCTCAGAGAT | |
| | GGGGACAGATATAGCCTGAGGCAGGACGGGACGAGGTGTGAGCTGCACATTCGTGGCC | |
| | TGGCTGTGGAGGACACTGGAGAGTATTTGTGTGTGTGCGGGCAGGAGAGAACCTCAGC | |
| | TACACTCACTGTCAGGGCCCTGCCAGCCAGATTCATAGACAACATGACAAACCACGPA | |
| | GCCAGAGAAGGGGCCACGGCCACACTGCACTGTGAACTGAGCAAGGTGGCCCCTGTGC | |
| | AGTGGAGGAAGGGACCTGAAACCCTCCGAGATGGGGACAGACACAGCCTGAGGCAGCA | |
| | TGGGTCCAGGTGTGAGCTGCAGATCCGTGGCCTGGCTGTGGTGGATGCCGGGCACTAC | |
| | TCGTGTGTGTGCGGGCAGGAGAGGACCTCAGCCACACTCACTGTCAGGGCCCTGCCTG | |
| | CCAGATTCATAGAAGATGTGAAAAACCAGGAGGCCAGAGAACGGGCCACGGCCGTGCT | |
| | GCAATGTGAGCTGAGCAAGGCGGCCCCCGTGGAGTGGAGGAAGGGGTCTGAGACCCTC | |
| | AGAGGTGGGGACAGATACAGCCTGAGGCAGGATGGGACCAGATGTGAGCTGCAGATTC | |
| | ATGGCCTGTCTGTGGCAGACACTGGGGAGTACTCGTGTGTGTGCGGGCAGCAGAGGAC | |
| | CTCGGCCACACTCACCGTCAGGGCCCTGCCTCCACGATTCACTCAAGATCTGAAGACC | |
| | AAGCAGGCCTCAGAAGGGGCCACAGCTACACTGCAGTGTGAGCTGAGCAAGGTGGCCC | |
| | CTGTGGAATGGAAGAAGGGTCCTGAGACCCTCAGAGATGGGGGCAGATACAGCCTGAA | |
| | GCAGGATGGGACGAGGTGTGAGCTGCAGATCCATGACCTGTCTGTGGCGGATGCTGGG | |
| | GAATACTCATGCATGTGTGGACAAGAGAGGACCTCGGCCACGCTCACTGTCAGGGACT | |
| | GCCACACTCTTCACGTCATGCCACACTATCCCTTCCAGCTTCCTGGGCTGCTGAACGA | |
| | ACCAGAAGAAACTCTCATCTACATCCAGATTCCCTCTCCTGTGATACTGTTCACAGAG | |
| | GGTCTGAGGAATGAAGAGGCCATGGAAGGGGCCACAGCCACACTCCAATGTGAGCTGA | |
| | GCAAGGCAGCCCCTGTGGAGTGGAGGAAAGGCCTTGAGGCTCTCAGAGATGGGGACAA | |
| | ATACAGCCTGAGACAAGACGGGGCTGTGTGTGAGCTGCAGATTCATGGCCTGGCTATG | |
| | CCAGATAACGGGGTGTACTCATCCCTGCCTGCCAGATTCATAGAGGATATGAGAAACC | |
| | AGAAGGCCACAGAAGGGGCTACAGTCACATTGCAATGTAACCTGAGAAAGGCGGCCCC | |
| | CGTGGAGTGGAGAAAGGGGCCCAACACCCTCAAAGATGGGGACAGGTACAGCCTGAAG | |
| | CAGGATGGGACCAGTTGTGAGCTGCAGATTCGTGGCCTGGTCATAGCAGATGCTGGAG | |
| | AATACTCGTGCATATGTGAGCAGGAGAGGACCTCGGCCACGCTCACTGTCAGGGCCCT | |
| | GCCCGCCAGATTCATAGAAGATGTGAGAAATCACGAGGCCACAGAAGGGGCCACAGCT | |
| | GTGCTGCAGTGTOAGCTGAGCAAGGCGGCCCCCGTGGAGTGGCGGAAGGGGTCTGAGA | |
| | CCCTCAGAGATGGGGACAGATATAGCCTGACGCAGGACGGGACGAGGTGTGAGCTGCA | |
| | CAAACCAGGAAGCCAGAGAAGGGGCCACGGCCACACTGCACTGTGAACTGAGCAAGGT | |
| | GGCCCCTGTGGAGTGGAGGAAGGGACCTGAAACCCTCCGAGATGGGGACAGACACAGC | |
| | CTGAGGCAGGATGGGACCAGGTGTGAGCTGCAGATTCGTGGCCTGTCTGTGGCAGATC | |
| | CCGGCGAGTACTCGTGCGTGTGTGGGCAGGAGAGGACCTCAGCCACACTCACGATCAG | |
| | GGCCCTGCCCGCCAAGTTCACAAAGCGTCTGAGGAATGAAGAGGCCACAGAAGGGGCC | |
| | ACGGCTATGTTGCAGTGTGAGCTGAGCAAGGTGGCCCCTGTTGAGTGGAGGAAGGGAC | |
| | CTGAAACCCTCAGAGATGGGGACAGATACAACCTGAGGCAGGATGGGACCAGATGTGA | |
| | GCTGCAGATTCATGOCCTGTCCGTGGCAGACACTGCGGAGTACTCATGTGTATGTGGT | |
| | CAGGAGAAGACGTCGGCCACTCTCACTGTCAAGGCCCCACAGCCAGTGTTCCGGGAGC | |
| | CGCTGCAGAGTCTGCAGGCGGAGGAGGGCTCCACGGCCACCCTGCAGTGTGAGCTGTC | |
| | TGAGCCCACTGCTACAGTGGTCTGGAGCAAGGGTGGCCTGCAGCTGCAGCCCAATGGG | |
| | CGCCGGGAGCCACGGCTTCAGGGCTGCACCGCGGAGCTGGTGTTACAGGACCTACAAC | |
| | GTGAAGACACTGGCGAATACACTTGCACCTGTGGCTCCCAGGCCACCAGTGCCACCCT | |
| | CACTGTCACAGCTGCGCCTGTGCGGTTCCTCCGAGAGCTGCAGCACCAGGAGGTGGAT | |
| | GAGGGAGGCACCGCACACTTATGCTGCGAGCTGAGCCGGGCGGGTGCGAGCGTGGAGT | |
| | GGCGCAAGGGCTCCCTACAGCTCTTCCCTTGTGCCAAGTACCAGATGGTGCAGGATGG | |
| | TGCAGCTGCAGAGCTGCTGGTACGCGGAGTGGACCAGGAGGATGCGGGTGACTACACG | |
| | TGTGACACGGGCCACACGCAGAGCATGGCCAGCCTCTCTGTCCGTGGAGGGCGTGGAG | |
| | CTGCATGCGGGCCCCAAGTACGAGATGCGGCGCAGGGGGCCACGCGGGAGCTGCTGAT | |
| | CCACCAACTGGAGGCCAAGGACACGGGCGAGTATGCCTGTGTGACAGGCGGCCAGAAA | |
| | ACCGCTGCCTCCCTCAGGGTCACAGAGCCTGAGGTGACCATTGTACGGGGGCTGGTTG | |
| | ATGCGGAGGTGACGGCCGATGAGGATGTTGAGTTCAGCTGTGAGGTGTCCAGGGCTGG | |
| | AGCCACAGGCGTGCAGTGGTGCCTACAGGGCCTGCCACTGCAAAGCAATGAGGTGACA | |
| | GAGCTGGCTGTGCGGGATGGCCGCATCCACACCCTGCGGCTGAAGGGCGTGACGCCCG | |
| | AGGACGCTGGCACTGTCTCCTTCCATTTGGGAAACCATGCTTCCTCTGCCCAGCTCAC | |
| | CGTCAGAGCTCCTGAGGTGACCATCCTGGAGCCCCTGCAGGACGTGCAGCTCAGAGGG | |
| | GTGCCCCTGCAGGCCAACGAGATGAATGACATCACTGTGGAGCAGGGCACACTCCACC | |
| | TGCTCACCCTGCACAAGGTGACCCTTGAGGATGCTGGAACTGTCAGTTTCCACGTGGG | |
| | CACGTGTAGCTCTGAGGCCCAGCTGAAAGTCACAGAGGCAGTGCCGTGCCTGGTACGT | |
| | GGCTTGCAGAATGTGGATGTCTTCGCGGGGGAGGTGGCCACGTTCTCCTGTGAGGATG | |
| | GCCCCCAGAGCGCCATCGCTGTCCGAGATGGGATCTTTCACTCCCTCATGCTCTCGGG | |
| | CCTGGGGGTGGCCGACTCCGGCACTGTCATCTTCCGCGCAGGGCCCCTGGTCTCCACG | |
| | GCCAAGTTGTTGATCAAAGATCCCGTGGTGGAGGTGGTCAGTGCCATGCAGGACTTGG | |
| | CCGTGGAGGAGGGTGGCTCGGCTGAGCTCCTCTGCCAGTATTCACGGCCCGTGCAGGC | |
| | CACGTGGAAGATGGACGAGCGGGAGGTGCACACGGATGGGCACCGTGTCATCATAGAG | |
| | CAGGACTGGAACGTGGCCAGGCTGACCTTCAGGCCGGCCTTGCCCTGTGACAGTGGCA | |
| | TCTATTCTTGTGAGGCTGCGGGCACCCGCGTAGTGGCCCTGCTGCAAGTCCAAGCCAA | |
| | GAACACGGTGGTGCGAGGGCTGGAGGATGTGGAGGCGCTGGAGGGCGGCGAGGCGCTG | |
| | TTCGAGTGCCAGCTGTCCCAGCCCGAGGTGGCCGCCCACACCTGGCTGCTGGACGACG | |
| | AACCCGTGCGCACCTCGGAGAACGCCGAGGTGGTCTTCTTCGAGAACGGCCTGCGCCA | |
| | CCTGCTGCTGCTCAAAAACTTGCGGCCACAAGACAGCTGCCGGGTGACCTTCCTGGCT | |
| | GGGGATATGGTGACGTCCGCATTCCTCACGGTCCGAGGTGACTGCGCTGTGCTGGTGC | |
| | AGGGCTGGCGCCTGGAGATCCTGGAGCCTCTGAAAAACGCGGCGGTCCGGGCCGGCGC | |
| | ACAGGCACGCTTCACCTGCACGCTCAOCGAGGCGGTGCCCGTGGGAGAGGCGTCCTGG | |
| | TACATCAATGGCGCGGCAGTGCAGCCGGATGACAGCGACTGGACTGTCACCGCCGACG | |
| | GCAGTCACCACGCCCTACTGCTGCGCAGCGCCCAGCCCCACCACGCCGGGGAGGTCAC | |
| | CTTCGCTTGCCGCGACGCCGTGGCCTCTGCGCGGCTCACCGTGCTGGGCCTCCCTGAT | |
| | CCCCCAGAGGATGCTGAGGTGGTGGCTCGCAGCAGCCACACTGTGACACTGTCTTGGG | |
| | CAGCTCCCATGAGTGATGGAGGCGGTGGTCTCTGTGCCTACCGCGTGGAGGTGAAGGA | |
| | GGGGGCCACAGGCCAGTGGCGGCTGTGCCACGAGCTCGTGCCTGGACCCGAGTGTGTG | |
| | GTGGATGGCCTGGCCCCCGGGGAGACCTACCGCTTCCGTGTGGCAGCTGTGGGCCCTG | |
| | TGGGTGCTGGGGAACCGGTTCACCTGCCCCAGACAGTGCGGCTTGAGCCACCGAAGCC | |
| | TGTGCCTCCCCAGCCCTCAGCCCCTGAGAGCCGGCAGGTGGCAGCTGGTGAAGATGTC | |
| | TGTCTGGAGCTTGAGGTGGTGGCTGAGGCTGGCGAGGTCATCTGGCACAAGGGAATGG | |
| | AGCGCATCCAGCCCGGTGGGCGGTTCGAGGTGGTCTCCCAGGGTCGGCAACAGATGCT | |
| | GGCTCCATCTGCCCTGCGGCTGCCACCTTCCAGGTGGCACTGAGCCCAGCCTCTGTGG | |
| | ATGAGGCCCCTCAGCCCAGCTTGCCCCCCGAGGCAGCCCAGGAGGGTGACCTGCACCT | |
| | ACTGTGGGAGGCCCTGGCTCGGAAACGTCGCATGAGCCGTGAGCCCACGCTGGACTCC | |
| | ATTAGCGAGCTGCCAGAGGAGGACGGCCGCTCGCAGCGCCTGCCACAGGAGGCAGAGG | |
| | AGGTGGCACCTGATCTCTCTGAAGGCTACTCCACGGCCGATGAGCTGGCCCGCACTGG | |
| | AGATGCTCACCTCTCACACACCAGCTCTGATGATGAGTCCCGGGCAGGCACCCCTTCC | |
| | CTGGTCACCTACCTCAAGAAGGCTGGGAGGCCAGGCACCTCACCACTGGCCAGCAAGG | |
| | TGAGCCCCCCCAACTTGGCCTGCAAGGAGAGGTTCCCCACGCCCCGGGCCGGCCCCAG | |
| | CCTCCTGGGCTTCGTGGGGGCAGACCCAGCCTTTCCCGGCAGCGAGCGCTCGGCCAGG | |
| | TGCACTAGGCGCTGTGCGGCCCCCCCTCCCCGCGAGTCCCTCAAGCGGGAACCTGCCT | |
| | CGTGTCTCCCAGGAGCCATGGAGGCTGTGGAACTCGCCAGAAAACTGCAGGAGGAAGC | |
| | TACGTGCTCCATCTGTCTGGATTACTTCACAGACCCTGTGATGACCACCTGTGGCCAC | |
| | AACTTCTGCCGAGCGTGCATCCAGCTGAGCTGGGAAAAGGCGAGGGGCAAGAAGGGGA | |
| | GGCGGAAGCGGAAGGGCTCCTTCCCCTGCCCCGAGTGCAGAGAGATGTCCCCGCAGAG | |
| | GAACCTGCTGCCCAACCGGCTGCTGACCAAGGTGGCCGAGATGGCGCAGCAGCATCCT | |
| | GGTCTGCAGAAGCAAGACCTGTGCCAGGAGCACCACGAGCCCCTCAAGCTTTTCTGCC | |
| | AGAAGGACCAGAGCCCCATCTGTGTGGTGTGCAGGGAGTCCCGGGAGCACCGGCTGCA | |
| | CAGGGTGCTGCCCGCCGAGCAGGCAGTGCAGGGGTACAAGTTGAAGCTGGAGGAGGAC | |
| | ATGGAGTACCTTCGGGAGCAGATCACCAGGACAGGGAATCTGCAGGCCAGGGAGGAGC | |
| | AGAGCTTAGCCGAGTGGCAGGGCAAGGTGAAGGAGCGGAGAGAACGCATTGTGCTGGA | |
| | GTTTGAGAAGATGAACCTCTACCTGGTGGAAGAAGAGCAGAGGCTCCTCCACGCTCTC | |
| | GAGACGGAAGAAGAGGAGACTGCCAGCAGGCTCCGGGAGAGCGTGGCCTGCCTGGACC | |
| | GGCAGGGTCACTCTCTGGAGCTGCTGCTGCTGCAGCTGGAGGAGCGGAGCACACAGGG | |
| | GCCCCTCCAGATGCTGCAGGACATGAAGGAACCCCTGAGCAGGGCGGCGTTACTGGTG | |
| | GTTCTAATTCATGGGATGAATCTTGTTGAGTTCCCAGTGGTCTCTCTGCCCAGCCCCC | |
| | TGTACCTTATTGCCACCAACCCCCACACACAATTGGGCCCGGGGACTCCCACCTTTGA | |
| | CCCTGAATGCCCCACACCTCTCCCCATCTCTCCACCACCACGCCCATCTACAGAGGAT | |
| | GTGGTGCCTGATGCCACCTCCGCGTACCCCTACCTCCTCCTGTATGAGAGCCGCCAGA | |
| | CGCGCTACCTCGGCTCTTCGCCGGAGGGCAGTGGGTTCTGCAGCAAGGACCGATTTGT | |
| | GCCTTACCCCTGTGCTGTGGGCCAGACGGCCTTCTCCTCTGGGAGGCACTACTGGGAG | |
| | GTGGGCATGAACATCACCGGGGACGCGTTGTGGGCCCTGGGTGTGTCCAGGGACAACG | |
| | TGAGCCGGAAAGACAGGGTCCCCAAGTGCCCCGAAAACGGCTTCTGGGTGGTGCAGCT | |
| | GTCCAAGGGGACCAAGTACTTATCCACCTTCTCTGCCCTAACCCCGGTCATGCTGATG | |
| | GAGCCTCCCAGCCACATGGGCATCTTCCTGGACTTCGAAGCCGGGGAAGTGTCCTTCT | |
| | ACAGTGTAAGCGATGGGTCCCACCTGCACACCTACTCCCAGGCCACCTTCCCAGGCCC | |
| | CCTGCAGCCTTTCTTCTGCCTGGGGGCTCCCAAGTCTGGTCAGATGGTCATCTCCACA | |
| | GTGACCATGGCAGGGGTAAAAGACCTGGCCACAAGAACCGGAGCGGTCGTGACGCCAG | |
| | CGCTCGGAGCCTACGCGCCCAGCGCTACCGAAACCCAGAGTCCTGCGCCCTGGAGTCC | |
| | CCGCGCCCCGGAGCCCGAGCACCCGGCAGTCCCGACCCTCGCCCCCCGGAGTGCCCGA | |
| | GCCTGCGCCGCCGCACCCGGATACCCCGGGTCCCCGCGAGCTCCCCAGGCCGCCCGCC | |
| | GCCGCCCCGCGGACAGTACCGCCTTCCTCCCCTCTGTCCGCGCCATGGCCGCCCCCGA | |
| | CCTGTCCACCAACCTCCAGGAGGAGGCCACCTGCGCCATCTGCCTCGACTACTTCACG | |
| | GATCCGGTGATGACCGACTGCGGCCACAACTTCTGCCGCGAGTGCATCCGGCGCTGCT | |
| | GGGGCCAGCCCGAGGGCCCGTACGCGTGCCCCGAGTGCCGCGAGCTGTCCCCGCAGAG | |
| | GAACCTGCGGCCCAACCGCCCGCTTGCTAAGATGGCCGAGATGGCGCGGCGCCTGCAC | |
| | CCGCCGTCGCCGGTCCCGCAGGGCGTGTGCCCCGCGCACCGCGAGCCACTGGCCGCCT | |
| | TCTGTGGCGACGAGCTGCGCCTCCTGTGTGCGGCCTGCGAGCGCTCTGGGGAGCACTG | |
| | GGCGCACCGCGTGCGGCCGCTGCAGGACGCGGCCGAAGACCTCAAGGCGAAGCTGGAG | |
| | AAGTCACTGGAGCATCTCCGGAAGCAGATGCAGGATGCGTTGCTGTTCCAAGCCCAGG | |
| | CGCATGAGACCTGCGTCTTGTGGCAGAAGATGGTGGAGAGCCAGCGGCAGAACGTGCT | |
| | GGGTGAGTTCGAGCGTCTTCGCCGTTTGCTGGCAGAGGGAGGGACAGCAGCTGCTGCA | |
| | GAGGCTGGAGAGGAGGAGCTGAAGCAGAGCGCCCACCTAGCTGAGCTCATCGCCGAGC | |
| | TCGAGAGGCCGCTGCCAGCTGCCTGCGCTGGGGCTGCTGCAGCAGAGTCTTTTCCCAT | |
| | GTGTGGGCTCCACTCCCTGAGCCGGCCCCCTGGCGTGGGCTTTCCTTGGTGCACCCCC | |
| | TGGAGCCCACAATGCTGCAGATGTGGCTGGGCGGCTTTGCACAGGGGGTGACACTGCT | |
| | GCCGGCCTCTGGAGCCCACCAGAACATCAGTCCAGGCACCGGCTCCTGGTTTCGATTG | |
| | TCATTTCTATTATTTAAGGGGTACAAGTGCAGTCAGAGTGTAGCCATCACCCCAATGG | |
| | TCCACACTGTACCCAAGACCAAACCCCCTTGTCGAGGCCAAGGTTCTCCTCTACCCCC | |
| | AAGCCCTTCTCCTGCCGCCCCTGCACCCGGCCTTGTGACAGCCACCACCTGTTTCCAA | |
| | ATGACACCAGGGGTGGGCCGCCCACCCCAGGACATCAAGGACGCCCTGCGCAGGGTCC | |
| | AGGATGTGAAGCTGCAGCCCCCAGAAGTTGTGCCTATGGAGCTGAGGACCGTGTGCAG | |
| | GGTCCCGGGACTGGTAGAGACACTGCGGAGGTTTCGAGGGGACGTGACCTTGGACCCG | |
| | GACACCGCCAACCCTGAGCTGATCCTGTCTGAAGACAGGCGGAGCGTGCAGCGGGGGG | |
| | ACCTACGGCAGGCCCTGCCGGACAGCCCAGAGCGCTTTGACCCCGGCCCCTGCGTGCT | |
| | GGGCCAGGAGCGCTTCACCTCAGGCCGCCACTACTGGGAGGTGGAGGTTGGGGACCGC | |
| | ACCAGCTGGGCCCTGGGGGTGTGCAGGGAGAACGTGAACAGGAAGGAGAAGGGCGAGC | |
| | TGTCCGCGGGCAACGGCTTCTGGATCCTGGTCTTCCTGGGGAGCTATTACAATTCCTC | |
| | GGAACGGGCCTTGGCTCCACTCCGGGACCCACCCAGGCGCGTGGGGATCTTTCTGGAC | |
| | TACOAGGCTGGACATCTCTCTTTCTACAGTGCCACCGATCGGTCACTGCTATTCATCT | |
| | TTCCCGAGATCCCCTTCTCGGGGACGCTGCGGCCCCTCTTCTCACCCCTGTCCAGCAG | |
| | CCCGACCCCGATGACTATCTGCCGGCCGAAAGGTGGGTCCGGGGACACCCTGGCTCCC | |
| | CAGTGACTCGGGCCCTCCTGGAGGA | |
| | ORF Start: ATG at 15 | ORF Stop: TGA at 14040 |
| | SEQ ID NO:74 | 4675 aa MW at 511097.3 kD |

| | |
|---|---|
| NOV13b, | MPLYNDSFHEISHKGRRHTLVLKSIQRADAGIVRASSLKVSTSARLEVRVKPVVFLKA |
| CG59237-02 Protein | LDDLSAEERGTLALQCEVSDPEAHVVWRKDGVQLGPSDKYDFLHTAGTRGLVVHDVSP |
| Sequence | EDAGLYTCHVGSEETRARVRVHDLHVGITKRLKTMEVLEGESCSFECVLSHESASDPA |
| | MWTVGGKTVGSSSRFQATRQGRKYILVVREAAPSDAGEVVFSVRGLTSKASLIVRERP |
| | AAIIKPLEDQWVAPCEDVELRCELSRAGTPVHWLKDRKAIRKSQKYDVVCEGTMAMLV |
| | IRGASLKDAGEYTCEVEASKSTASLHVEEKANCFTEELTNLQVEEKGTAVFTCKTEHP |
| | AATVTWRKGLLELPASGKHQPSQEGLTLRLTISALEKADSDTYTCDIGQAQSRAQLLV |
| | QGRRVHIIEDLEDVDVQEGSSATFRCRISPANYEPVHWFLDKTPLHANELNEIDAQPG |
| | GYHVLTLRQLALKDSGTIYFEAGDQRASAALRVTEKPSVFSRELTDATITEGEDLTLV |
| | CETSTCDIPVCWTKDGKTLRGSARCQLSHEGHRAQLLITGATLQDSGRYKCEAGGACS |
| | SSIVRVHARPVRFQEALKDLEVLEGGAATLRCVLSSVAAPVKWCYGNNVLRPGDKYSL |
| | RQEGAMLELVVRNLRPQDSGRYSCSFGDQTTSATLTVTALPAQFIGKLRNKEATEGAT |
| | ATLRCELSKAAPVEWRKGSETLRDGDRYCLRQDGAMCELQIRGLAMVDAAEYSCVCGE |
| | ERTSASLTIRPMPAHFIGRLRHQESIEGATATLRCELSKAAPVEWRKGRESLRDGDRH |
| | SLRQDGAVCELQICGLAVADAGEYSCVCGEERTSATLTVKALPAKFTEGLRNEEAVEG |
| | ATANLWCELSKVAPVEWRKGPENLRDGDRYILRQEGTRCELQICGLAHADAGEYLCVC |
| | GQERTSATLTIRALPARFIEDVKNQEAREGATAVLQCELNSAAPVEWRKGSETLRDGD |
| | RYSLRQDGTKCELQIRGLAMADTGEYSCVCGQERTSAMLTVRALPIKFTEGLRNEEAT |
| | EGATAVLRCELSKMAPVEWWKGHETLRDGDRHSLRQDGARCELQIRGLVAEDAGEYLC |
| | MCGKERTSAMLTVRAMPSKEIEGLRNEEATEGDTATLWCELSKAAPVEWRKGHETLRD |
| | GDRHSLRQDGSRCELQIRGLAVVDAGEYSCVCGQERTSATLTVRALPARFIEDVKNQE |
| | AREGATAVLQCELSKAAPVEWRKGSETLRGGDRYSLRQDGTRCELQIHGLSVADTGEY |
| | SCVCGQERTSATLTVRALPARFTQDLKTKEASEGATATLQCELSKVAPVEWKKGPETL |
| | RDGGRYSLKQDGTRCELQIHDLSVADAGEYSCMCGQERTSATLTVRALPARFTEGLRN |
| | EEAMEGATATLQCELSKAAPVEWRKGLEALRDGDKYSLRQDGAVCELQIHGLAMADNG |
| | VYSCVCGQERTSATLTVRALPARFIEDMRNQKATEGATVTLQCKLRKAAPVEWRKGPN |
| | TLKDGDRYSLKQDGTSCELQIRGLVIADAGEYSCICEQERTSATLTVRALPARFIEDV |
| | RNHEATEGATAVLQCELSKAAPVEWRKGSETLRDGDRYSLRQDGTRCELQIRGLAVED |
| | TGEYLCVCGQERTSATLTVRALPARFIDNNTNQEAREGATATLHCELSKVAPVEWRKG |
| | PETLRDGDRHSLRQDGSRCELQIRGLAVVDAGEYSCVCGQERTSATLTVRALPARFIE |
| | DVKNQEAREGATAVLQCELSKAAPVEWRKGSETLRGGDRYSLRQDGTRCELQIHGLSV |
| | ADTGEYSCVCGQERTSATLTVRALPARFTQDLKTKEASEGATATLQCELSKVAPVEWK |
| | KGPETLRDGGRYSLKQDGTRCELQIHDLSVADAGEYSCMCGQERTSATLTVRDCHTLH |
| | VMPHYPFQLPGLLKEPEETLIYIQIPSPVILFTEGLRNEEANEGATATLQCELSKAAP |
| | VEWRKGLEALRDGDKYSLRQDGAVCELQIHGLAMADNGVYSSLPARFIEDMRNQKATE |
| | GATVTLQCKLRKAAPVEWRKGPNTLKDGDRYSLKQDGTSCELQIRGLVIADAGEYSCI |
| | CEQERTSATLTVRALPARFIEDVRNEEATEGATAVLQCELSKAAPVEWRKGSETLRDG |
| | DRYSLRQDGTRCELQIRGLAVEDTGEYLCVCGQERTSATLTVRALPARFIDNNTNQEA |
| | REGATATLHCELSKVAPVEWRKGPETLRDGDRHSLRQDGTRCELQIRGLSVADAGEYS |
| | CVCGQERTSATLTIRALPAKFTKGLRNEEATEGATANLQCELSKVAPVEWRKGPETLR |
| | DGDRYNLRQDGTRCELQIHGLSVADTGEYSCVCGQEKTSATLTVKAPQPVFREPLQSL |
| | QAEEGSTATLQCELSEPTATVVWSKGGLQLQANGRREPRLQGCTAELVLQDLQREDTG |
| | EYTCTCGSQATSATLTVTAAPVRFLRELQHQEVDEGGTAHLCCELSRAGASVEWRKGS |
| | LQLFPCAKYQMVQDGAAAELLVRGVEQEDAGDYTCDTGHTQSMASLSVRGGRGAACGP |
| | QVRDAAQGATRELLIHQLEAKDTGEYACVTGGQKTAASLRVTEPEVTIVRGLVDAEVT |
| | ADEDVEFSCEVSRAGATGVQWCLQGLPLQSNEVTEVAVRDGRIHTLRLKGVTPEDAGT |
| | VSFHLGNUASSAQLTVRAPEVTILEPLQDVQLRCVPLQANEMNDITVEQGTLHLLTLH |
| | KVTLEDAGTVSFHVGTCSSEAQLKVTEAVPCLVRGLQNVDVFAGEVATFSCEDGPQSA |
| | IAVRDGIFHSLMLSGLGVADSGTVIFRAGPLVSTAKLLIKDPVVEVVSAMQDLAVEEG |
| | GSAELLCQYSRPVQATWKMDEREVHTDGHRVIIEQDWNVARLTFRPALPCDSGIYSCE |
| | AAGTRVVALLQVQAKNTVVRGLENVEALEGGEALFECQLSQPEVAAHTWLLDDEPVRT |
| | SENAEVVFFENGLRHLLLLKNLRPQDSCRVTFLAGDMVTSAFLTVRGDCAVLVQGWRL |
| | EILEPLKNAAVRAGAQARFTCTLSEAVPVGEASWYINCAAVQPDDSDWTVTADGSHHA |
| | LLLRSAQPHHAGEVTFACRDAVASARLTVLGLPDPPEDAEVVARSSHTVTLSWAAPMS |
| | DGGGGLCGYRVEVKEGATGQWRLCHELVPGPECVVDGLAPGETYRFRVAAVGPVGAGE |
| | PVHLPQTVRLEPPKPVPPQPSAPESRQVAAGEDVCLELEVVAEAGEVIWHKGMERIQP |
| | GGRFEVVSQGRQQMLVIKGFTAEDQGEYHCGLAQGSICPAAATFQVALSPASVDEAPQ |
| | PSLPPEAAQEGDLHLLWEALARKRRMSREPTLDSISELPEEDGRSQRLPQEAEEVAPD |
| | LSEGYSTADELARTGDADLSHTSSDDESRAGTPSLVTYLKKAGRPGTSPLASKVSPPN |
| | LACKERFPTPRAGRSLLGFVGADPAFPGSERSARCTRRCAAPPPRESLKREPASCLPG |
| | AMEAVELARKLQEEATCSICLDYFTDPVNTTCGHNFCRACIQLSWEKARGKKGRRKRK |
| | CSFPCPECREMSPQRNLLPNRLLTKVAEMAQQHPGLQKQDLCQEHHEPLKLFCQKDQS |
| | PICVVCRESREHRLHRVLPAEEAVQGYKLKLEEDMEYLREQITRTGNLQAREEQSLAE |
| | WQGKVKERRERIVLEFEKMNLYLVEEEQRLLQALETEEEETASRLRESVACLDRQGHS |
| | LELLLLQLEERSTQGPLQMLQDMKEPLSRAALLVVLIHGMNLVEFPVVSLPSPLYLIA |
| | TKAHTQLGPGTPTFDPECPTPLPISPPPRPSTEDVVPDATSAYPYLLLYESRQRRYLG |
| | SSPEGSGFCSKDRFVAYPCAVCQTAFSSGRHYWEVGMNITGDALWALGVCRDNVSRKD |
| | RVPKCPENGFWVVQLSKGTKYLSTFSALTPVMLMEPPSHMGIFLDFEAGEVSFYSVSD |
| | GSHLHTYSQATFPGPLQPFFCLGAPKSGQMVISTVTMAGVKDLATRTGAVVTPALGAY |
| | APSATETQSPAPWSPRAPEPEHPGVPSLAPRSARACAAAPGYPGSPRAAEAARRRPAD |
| | STAFLPSVRANAAPDLSTNLQEEATCAICLDYFTDPVMTDCGHNFCRECIRRCWGQPE |
| | GPYACPECRELSPQRNLRPNRPLAKMAEMARRLHPPSPVPQGVCPAHREPLAAFCGDE |
| | LRLLCAACERSGEHWAHRVRPLQDAAEDLKAKLEKSLEHLRKQMQDALLFQAQADETC |
| | VLWQKNVESQRQNVLGEFERLRRLLAEGGTAAAAEAGEEELKQSAHLAELIAELERPL |
| | PAACAGAAAGESFPMCGLHSLSRPPGVGFPWCTPKPEPVDALACAWRQGCQTQVEPTM |
| | LQMWLGGFAQGVTLLPASGAQQNISPGTGSWERLSFLLFKGYKCSQSVAITRMVHTVP |
| | KTKPPCRGQGSPLPPSPSPAAPAPGLVTATTCFQMTPGVCRPPQDIKDALRRVQDVKL |
| | QPPEVVPMELRTVCRVPGLVETLRRFRGDVTLDPDTANPELILSEDRRSVQRGDLRQA |
| | LPDSPERFDPGPCVLGQERFTSGRHYWEVEVGDRTSWALGVCRENVNRKEKGELSAGN |
| | GFWILVFLGSYYNSSERALAPLRDPPRRVGIFLDYEACHLSFYSATDGSLLFIFPEIP |
| | FSGTLRPLFSPLSSSPTPMTICRPKGGSGDTLAPQ |

Sequence comparison of the above protein sequences yields the following sequence relationships shown in Table 13B.

TABLE 13B
Comparison of NOV13a against NOV13b and NOV13c.

| | NOV13a Residues/ | Identities/Similarities |
|---|---|---|
| Protein Sequence | Match Residues | for the Matched Region |
| NOV13b | 1 . . . 4691 | 4399/4696 (93%) |
| | 1 . . . 4675 | 4403/4696 (93%) |

Further analysis of the NOV13a protein yielded the following properties shown in Table 13C.

TABLE 13C
Protein Sequence Properties NOV13a

| | |
|---|---|
| PSort | 0.8500 probability located in endoplasmic reticulum |
| analysis: | (membrane); 0.4400 probability located in plasma membrane; |
| | 0.3500 probability located in nucleus; 0.3000 |
| | probability located in microbody (peroxisome) |
| SignalP | No Known Signal Sequence Indicated |
| analysis: | |

A search of the NOV13a protein against the Geneseq database, a proprietary database that contains sequences published in patents and patent publication, yielded several homologous proteins shown in Table 13D.

TABLE 13D
Geneseq Results for NOV13a

| | | NOV13a | Identities/ | |
|---|---|---|---|---|
| | | Residues/ | Similarities for | |
| Geneseq | Protein/Organism/Length/[Patent#, | Match | the Matched | Expect |
| Identifier | Date] | Residues | Region | Value |
| AAB42919 | Human ORFX ORF2683 polypeptide | 3482 . . . 3981 | 448/500 (89%) | 0.0 |
| | sequence SEQ ID NO:5366 - Homo | 1 . . . 473 | 452/500 (89%) | |
| | sapiens, 477 aa. [WO200058473-A2, | | | |
| | 05-OCT-2000] | | | |
| AAY53666 | from an alignment with protein 608 - | 264 . . . 3443 | 1270/3235 (38%) | 0.0 |
| | Unidentified, 4412 aa. [WO9960164- | | | |
| | A1, 25-NOV-1999] | | | |
| AAU05396 | Human titin (connectin) protein | 9 . . . 1892 | 526/1918 (27%) | 0.0 |
| | sequence - Homo sapiens, 26926 aa. | 4667 . . . 6570 | 859/1918 (44%) | |
| | [WO200151666-A1, 19-JUL-2001] | | | |
| AAB43498 | Human cancer associated protein | 3480 . . . 3992 | 193/536 (36%) | 5e−84 |
| | sequence SEQ ID NO:943 - Homo | 66 . . . 570 | 285/536 (53%) | |
| | sapiens, 580 aa. [WO200055350-A1, | | | |
| | 21-SEP-2000] | | | |
| AAY53667 | Sequence gi/3328186 from an | 70 . . . 2452 | 586/2595 (22%) | 3e−80 |
| | alignment with protein 608 - | 446 . . . 2900 | 977/2595 (37%) | |
| | Unidentified, 3117 aa. [WO9960164- | | | |
| | A1, 25-Nov-1999] | | | |

In a BLAST search of public sequence databases, the NOV13a protein was found to have homology to the proteins shown in the BLASTP data in Table 13E.

TABLE 13E
Public BLASTP Results for NOV13a

| | | NOV13a | | |
|---|---|---|---|---|
| Protein | | Residues/ | Identities/ | |
| Accession | | Match | Similarities for the | Expect |
| Number | Protein/Organisation/Length | Residues | Matched Portion | Value |
| Q9HCL6 | KIAA1556 PROTEIN - Homo | 132 . . . 1697 | 1565/1566 (99%) | 0.0 |
| | sapiens (Human), 1596 aa | 1 . . . 1566 | 1566/1566 (99%) | |
| | (fragment). | | | |
| Q96AA2 | OBSCURIN - Homo sapiens | 1 . . . 2368 | 1605/2430 (66%) | 0.0 |
| | (Human), 6620 aa. | 2595 . . . 4992 | 1764/2430 (72%) | |
| Q9Y577 | RING FINGER PROTEIN TERF - | 3482 . . . 3981 | 449/500 (89%) | 0.0 |
| | Homo sapiens (Human), 477 aa. | 1 . . . 473 | 453/500 (89%) | |
| CAD12456 | TITIN - Homo sapiens (Human), | 41 . . . 3323 | 811/3501 (23%) | 0.0 |
| | 34350 aa. | 5588 . . . 8921 | 1361/3501 (38%) | |
| Q9WV59 | RING FINGER PROTEIN TERF - | 3482 . . . 3981 | 341/500 (68%) | 0.0 |
| | Rattus norvegicus (Rat), 477 aa. | 1 . . . 473 | 400/500 (79%) | |

PFam analysis indicates that the NOV13a protein contains the domains shown in the Table 13F.

TABLE 13F
Domain Analysis of NOV13a

| | | Similarities | |
|---|---|---|---|
| | | for the Matched | |
| Pfam Domain | Region | Region | Value |
| ig: domain 1 of 34 | 67 . . . 26 | 18/64 (28%) | 1.3e−09 |
| | | 47/64 (73%) | |
| ig: domain 2 of 34 | 156 . . . 212 | 12/59 (20%) | 30 |
| | | 40/59 (68%) | |
| PEP-utilizers: domain 1 of | 178 . . . 260 | 22/105 (21%) | 2.4 |
| 1 | | 50/105 (48%) | |
| ig: domain 3 of 34 | 247 . . . 306 | 18/64 (28%) | 6.5e−10 |
| | | 43/64 (67%) | |
| MAM: domain 1 of 1 | 159 . . . 306 | 35/191 (18%) | 4.1 |
| | | 89/191 (47%) | |
| ig: domain 4 of 34 | 336 . . . 395 | 16/64 (25%) | 5.7e−07 |
| | | 43/64 (67%) | |
| ig: domain 5 of 34 | 425 . . . 481 | 13/60 (22%) | 0.0025 |
| | | 39/60 (65%) | |
| ig: domain 6 of 34 | 516 . . . 575 | 18/64 (28%) | 1.5e−08 |
| | | 47/64 (73%) | |
| ig: domain 7 of 34 | 605 . . . 664 | 15/64 (23%) | 1.1e−07 |
| | | 41/64 (64%) | |
| ig: domain 8 of 34 | 694 . . . 752 | 13/64 (20%) | 6.2e−05 |
| | | 42/64 (66%) | |
| ig: domain 9 of 34 | 782 . . . 840 | 14/64 (22%) | 1e−05 |
| | | 42/64 (66%) | |
| ig: domain 10 of 34 | 870 . . . 928 | 13/64 (20%) | 0.0012 |
| | | 40/64 (62%) | |
| ig: domain 11 of 34 | 958 . . . 1016 | 12/64 (19%) | 0.00011 |
| | | 44/64 (69%) | |
| ig: domain 12 of 34 | 1046 . . . 1104 | 13/64 (20%) | 8.5e−06 |
| | | 44/64 (69%) | |
| ig: domain 13 of 34 | 1134 . . . 1192 | 14/64 (22%) | 3.3e−06 |
| | | 46/64 (72%) | |
| ig: domain 14 of 34 | 1222 . . . 1280 | 13/64 (20%) | 4.5e−06 |
| | | 45/64 (70%) | |
| ig: domain 15 of 34 | 1310 . . . 1368 | 13/64 (20%) | 8.5e−07 |
| | | 46/64 (72%) | |
| ig: domain 16 of 34 | 1398 . . . 1456 | 14/64 (22%) | 3e−05 |
| | | 41/64 (64%) | |
| ig: domain 17 of 34 | 1486 . . . 1544 | 13/64 (20%) | 0.00085 |
| | | 41/64 (64%) | |
| ig: domain 18 of 34 | 1574 . . . 1632 | 14/64 (22%) | 8.6e−06 |
| | | 44/64 (69%) | |
| ig: domain 19 of 34 | 1662 . . . 1720 | 14/64 (22%) | 3.6e−05 |
| | | 44/64 (69%) | |
| ig: domain 20 of 34 | 1750 . . . 1808 | 13/64 (20%) | 4.5e−06 |
| | | 45/64 (70%) | |
| ig: domain 21 of 34 | 1838 . . . 1896 | 13/64 (20%) | 8.5e−07 |
| | | 46/64 (72%) | |
| ig: domain 22 of 34 | 1958 . . . 2013 | 12/61 (20%) | 0.014 |
| | | 39/61 (64%) | |
| ig: domain 23 of 34 | 2031 . . . 2089 | 13/64 (20%) | 0.00085 |
| | | 41/64 (64%) | |
| ig: domain 24 of 34 | 2119 . . . 2177 | 14/64 (22%) | 8.6e−06 |
| | | 44/64 (69%) | |
| ig: domain 25 of 34 | 2207 . . . 2265 | 14/64 (22%) | 5.3e−06 |
| | | 45/64 (70%) | |
| ig: domain 26 of 34 | 2295 . . . 2353 | 13/64 (20%) | 1.4e−05 |
| | | 45/64 (70%) | |
| ig: domain 27 of 34 | 2383 . . . 2442 | 16/64 (25%) | 1e−09 |
| | | 48/64 (75%) | |
| ig: domain 28 of 34 | 2472 . . . 2531 | 14/64 (22%) | 0.0014 |
| | | 39/64 (61%) | |
| ig: domain 29 of 34 | 2565 . . . 2582 | 7/19 (37%) | 0.92 |
| | | 13/19 (68%) | |
| ig: domain 30 of 34 | 2612 . . . 2668 | 16/60 (27%) | 5.5e−05 |
| | | 42/60 (70%) | |
| ig: domain 31 of 34 | 2842 . . . 2901 | 16/65 (25%) | 0.00035 |
| | | 46/65 (71%) | |
| ig: domain 32 of 34 | 2930 . . . 2988 | 13/66 (20%) | 0.18 |
| | | 38/66 (58%) | |
| ig: domain 33 of 34 | 3030 . . . 3089 | 13/62 (21%) | 0.22 |
| | | 42/62 (68%) | |
| fn3: domain 1 of 1 | 3107 . . . 3192 | 29/89 (33%) | 1.2e−14 |
| | | 63/89 (71%) | |
| ig: domain 34 of 34 | 3237 . . . 3280 | 10/47 (21%) | 4.6 |
| | | 29/47 (62%) | |
| zf-C3HC4: domain 1 of 2 | 3497 . . . 3546 | 20/61 (33%) | 2.1e−14 |
| | | 48/61 (79%) | |
| PHD: domain 1 of 1 | 3496 . . . 3549 | 13/59 (22%) | 1.9 |
| | | 37/59 (63%) | |
| zf-B_box: domain 1 of 2 | 3575 . . . 3616 | 22/48 (46%) | 3.1e−15 |
| | | 38/48 (79%) | |
| zf-UBR1: domain 1 of 1 | 3578 . . . 3629 | 13/80 (16%) | 9.8 |
| | | 34/80 (42%) | |
| SPRY: domain 1 of 2 | 3856 . . . 3980 | 41/157 (26%) | 1.9e−32 |
| | | 95/157 (61%) | |
| zf-C3HC4: domain 2 of 2 | 4086 . . . 4116 | 16/41 (39%) | 3.8e−12 |
| | | 28/41 (68%) | |
| Flu_M1: domain 1 of 1 | 4178 . . . 4189 | 6/12 (50%) | 4.7 |
| | | 9/12 (75%) | |
| zf-B_box: domain 2 of 2 | 4156 . . . 4197 | 18/48 (38%) | 0.031 |
| | | 26/48 (54%) | |
| Idh_C: domain 1 of 1 | 4222 . . . 4242 | 8/21 (38%) | 8.9 |
| | | 17/21 (81%) | |
| SPRY: domain 2 of 2 | 4562 . . . 4681 | 36/157 (23%) | 5.6e−29 |
| | | 90/157 (57%) | |

### Example 14

The NOV14 clone was analyzed, and the nucleotide and polypeptide sequences are shown in Table 14A.

TABLE 14A
NOV14 Sequence Analysis

| | SEQ ID NO:75 | 1617 bp |
|---|---|---|
| NOV14a, | CTCTCTCGCCGGTGACCCGGTGTGCGTGGGGTCGAGGCGCCGGGCGGAGTGGCTCCGG | |
| CG58575-01 DNA | GCCGAAACGCCATGCGGAGGGGCGAGCGCAGGGACGCCGGAGGTCCGCGGCCCGAGTC | |
| Sequence | CCCGGTGCCCGCGGGCAGGGCCTCCCTGGAGGAGCCGCCTGACCGCCCGTCTGCCGGC | |
| | CAAGCCACCGGGCCGGGGCGAGGCCGCAGCACCGAGTCCGAGGTCTACGACGACGGCA | |
| | CCAACACCTTCTTCTGGCGAGCCCACACCTTAACCGTGCTCTTCATCCTCACCTGTAC | |
| | GCTTGGCTATGTGACGCTGCTGGAGGAAACACCTCAGGACACGGCCTACAACACCAAG | |
| | AGAGGTATTGTGGCCAGTATTTTGGTTTTCTTATGTTTTGGAGTCACACAAGCTAAAG | |
| | ACGGGCCATTTTCCAGACCTCATCCAGCTTACTGGAGGTTTTGGCTCTGCGTGAGTGT | |
| | GGTCTACGAGCTGTTTCTCATCTTTATACTCTTCCAGACTGTCCACGACGGCCGGCAG | |
| | TTTCTAAAGTATGTTGACCCCAAGCTGGGAGTCCCACTGCCAGAGAGAGACTACGGGG | |
| | GAAACTGCCTCATCTACGACCCAGACAATGAGACTGACCCCTTTCACAACATCTGGGA | |
| | CAAGTTGGATGGCTTTGTTCCCGCGCACTTTCTTGGCTGGTACCTGAAGACCCTGATG | |
| | ATCCGAGACTGGTGGATGTGCATGATCATCAGCGTGATGTTCGAGTTCCTGGAGTACA | |
| | GCCTGGAGCACCAGCTGCCCAACTTCAGCGAGTGCTGGTGGGATCACTGGATCATGGA | |
| | CGTGCTCGTCTGCAACGGGCTGGGCATCTACTGCGGCATGAAGACCCTTGAGTGGCTG | |
| | TCCCTGAAGACGTACAAGGGCAAGATGAAGAGGATCGCCTTCCAGTTCACGCCGTACA | |
| | GCTGGGTTCGCTTCGAGTGGAAGCCGGCCTCCAGCCTGCGTCGCTGGCTGGCCGTGTG | |
| | CGGCATCATCCTGGTGTTCCTGTTGGCAGAACTGAACACGTTCTACCTGAAGTTTGTG | |
| | CTGTGGATGCCCCCGGAGCACTACCTGGTCCTCCTGCGGCTCGTCTTCTTCGTGAACG | |
| | TGGGTGGCGTGGCCATGCGTGAGATCTACGACTTCATGGATGACCCGAAGCCCCACAA | |
| | GAAGCTGGGCCCGCAGGCCTGGCTGGTGGCGGCCATCACGGCCACGGAGCTGCTCATC | |
| | GTGGTGAAGTACGACCCCCACACGCTCACCCTGTCCCTGCCCTTCTACATCTCCCAGT | |
| | GCTGGACCCTCGGCTCCGTCCTGGCGCTCACCTGGACCGTCTGGCGCTTCTTCCTGCG | |
| | GGACATCACATTGAGGTACAAGGAGACCCGGTGGCAGAAGTGGCAGAACAAGGATGAC | |
| | CAGGGCAGCACCGTCGGCAACGGGGACCAGCACCCACTGGGGCTGGACGAAGACCTGC | |
| | TGGGGCCTGGGGTGGCCGAGGGCGAGGGAGCACCAACTCCAAACTGACCTGGGCCGTG | |
| | GCTGCCTCGTGAGCCTCCCAGAGCCCAGGCCTCCGTGGCCTCCTCCTGTCTCGTGTGG | |
| | GCGCTCGTCCACAAACACTCCGTGGCTGAGAGGCAGCGGATCCAGGCAGCG | |
| | ORF Start: ATG at 70 | ORF Stop: TCA at 1495 |
| | SEQ ID NO: 76 | 475 aa MW at 54735.7 kD |

| | |
|---|---|
| NOV14a, | MRRCERRDAGGPRPESPVPACRASLEEPPDCPSAGQATGPGRCRSTESEVYDDGTNTF |
| C058575-01 Protein | FWRAHTLTVLFILTCTLGYVTLLEETPQDTAYNTKRGIVASILVFLCFGVTQAKDGPF |
| Sequence | SRPHPAYWRFWLCVSVVYELFLIFILFQTVQDGRQFLKYVDPKLGVPLPERDYGGNCL |
| | IYDPDNETDPFHNIWDKLDGFVPAHFLGWYLKTLMIRDWWMCMIISVMFEFLEYSLEH |
| | QLPNFSECWWDHWIMDVLVCNGLGIYCGMKTLEWLSLKTYKGKMKRIAFQFTPYSWVR |
| | FEWKPASSLRRWLAVCGIILVFLLAELNTFYLKFVLWMPPEHYLVLLRLVFFVNVGGV |
| | AMREIYDFMDDPKPHKKLGPQAWLVAAITATELLIVVKYDPHTLTLSLPFYISQCWTL |
| | GSVLALTWTVWRFFLRDITLRYKETRWQKWQNKDDQGSTVGNGDQHPLGLDEDLLGPG |
| | VAEGEGAPTPN |

Further analysis of the NOV14a protein yielded the following properties shown in Table 14B.

TABLE 14B
Protein Sequence Properties NOV14a

| | |
|---|---|
| PSort | 0.6000 probability located in plasma membrane; 0.4000 |
| analysis: | probability located in Golgi body; 0.3000 probability located |
| | in endoplasmic reticulum (membrane); 0.0300 |
| | probability located in mitochondrial inner membrane |
| SignalP | Cleavage site between residues 8 and 9 |
| analysis: | |

A search of the NOV14a protein against the Geneseq database, a proprietary database that contains sequences published in patents and patent publication, yielded several homologous proteins shown in Table 14C.

TABLE 14C
Geneseq Results for NOV14a

| | | NOV14a | Identities/ | |
|---|---|---|---|---|
| Geneseq | Protein/Organism/Length [Patent #, | Match | the Matched | Expect |
| Identifier | Date] | Residues | Region | Value |
| AAM79907 | Human protein SEQ ID NO 3553 - | 1 . . . 475 | 474/487 (97%) | 0.0 |
| | Homo sapiens, 529 aa. | 19 . . . 505 | 474/487 (97%) | |
| | [WO200157190-A2, 09-AUG-2001] | | | |
| AAM78923 | Human protein SEQ ID NO 1585 - | 1 . . . 475 | 474/487 (97%) | 0.0 |
| | Homo sapiens, 487 aa. | 1 . . . 487 | 474/487 (97%) | |
| | [WO200157190-A2, 09-AUG-2001] | | | |
| AAB73515 | Human transferase HTFS-22, SEQ ID | 1 . . . 475 | 473/487 (97%) | 0.0 |
| | NO: 22 - Homo sapiens, 487 aa. | 1 . . . 487 | 473/487 (97%) | |
| | [WO200132888-A2, 10-MAY-2001] | | | |
| AAG30975 | Arabidopsis thaliana protein fragment | 41 . . . 387 | 141/363 (38%) | 2e−73 |
| | SEQ ID NO: 37124 - Arabidopsis | 3 . . . 363 | 209/363 (56%) | |
| | thaliana, 436 aa. [EP1033405-A2, | | | |
| | 06-SEP-2000] | | | |
| AAG30974 | Arabidopsis thaliana protein fragment | 41 . . . 387 | 141/363 (38%) | 2e−73 |
| | SEQ ID NO: 37123 - Arabidopsis | 21 . . . 381 | 209/363 (56%) | |
| | thaliana, 454 aa. [EP1033405-A2, | | | |
| | 06-SEP-2000] | | | |

In a BLAST search of public sequence databases, the NOV14a protein was found to have homology to the proteins shown in the BLASTP data in Table 14D.

TABLE 14D
Public BLASTP Results for NOV14a

| | | NOV14a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9BVG9 | SIMILAR TO | 1 . . . 475 | 474/487 (97%) | 0.0 |
| | PHOSPHATIDYLSERINE SYNTHASE | 1 . . . 487 | 474/487 (97%) | |
| | 2 - Homo sapiens (Human), 487 aa. | | | |
| O08888 | PHOSPHATIDYLSERINE SYNTHASE | 1 . . . 471 | 408/482 (84%) | 0.0 |
| | II - Cricetulus griseus (Chinese hamster), | 1 . . . 458 | 422/482 (86%) | |
| | 474 aa. | | | |
| Q9Z1X2 | PHOSPHATIDYLSERINE SYNTHASE- | 1 . . . 470 | 409/481 (85%) | 0.0 |
| | 2 - Mus musculus (Mouse), 473 aa. | 1 . . . 457 | 419/481 (87%) | |
| Q922A1 | SIMILAR TO | 95 . . . 470 | 345/387 (89%) | 0.0 |
| | PHOSPHATIDYLSERINE SYNTHASE | 1 . . . 384 | 354/387 (91%) | |
| | 2 - Mus musculus (Mouse), 400 aa | | | |
| | (fragment). | | | |
| Q9CY68 | 7 DAYS EMBRYO CDNA, RIKEN | 1 . . . 273 | 239/273 (87%) | e−144 |
| | FULL-LENGTH ENRICHED | 1 . . . 252 | 243/273 (88%) | |
| | LIBRARY, CLONE: C430041K09, | | | |
| | FULL INSERT SEQUENCE - Mus | | | |
| | musculus (Mouse), 300 aa. | | | |

PFam analysis that the NOV14a protein contains the domains shown in the Table 14E.

TABLE 14E
Domain Analysis of NOV14a

| | | Identities/ | |
|---|---|---|---|
| | | Similarities | |
| | NOV14a Match | for the Matched | Expect |
| Pfam Domain | Region | Region | Value |
| Peptidase_C20: domain | 302 . . . 327 | 8/28 (29%) | 2 |
| 1 of 1 | | 19/28 (68%) | |
| PSS: domain 1 of 1 | 119 . . . 390 | 151/310 (49%) | 3e−201 |
| | | 269/310 (87%) | |

### Example 15

The NOV15 clone was analyzed, and the nucleotide and polypeptide sequences are shown in Table 15A.

TABLE 15A
NOV15 Sequence Analysis

| | SEQ ID NO:77 | 1210 bp |
|---|---|---|
| NOV15a, | TCGCTCACCCACCCGGACTCATTCTCCCCAGACGCCAAGGATGGTGGTCATGGCACCC | |
| CG59256-01 DNA | CGAACCCTCTTCCTGCTACTCTCGGGGGCCCTGACCCTGACCGAGACCTGCGCGGGCT | |
| Sequence | CCCACTCCATGAGGTATTTCAGCGCCGCCGTGTCCCGGCCCGGCCGCGGGGAGCCCCG | |
| | CTTCATCGCCATGGGCTACGTGGACGACACGCAGTTCGTGCGGTTCGACAGCGACTCG | |
| | GCGTGTCCGAGGATGGAGCCGCGGGCGCCGTGGGTGGAGCAGGAGGGGCCAGAGTATT | |
| | GGGAAGAGGAGACACGGAACACCAAGGCCCACGCACAGACTGACAGAATGAACCTGCA | |
| | GACCCTGCGCGGCTACTACAACCAGAGCGAGGGGGTGGGGCCAGGTTCTCATACCCTC | |
| | CAGTGGATGATTCGCTGCGACCTGGGGTCCGACGGACGCCTCCTCCGCGGGTATGAAC | |
| | AGTATGCCTACGATGGCAACGATTACCTCCCCCTGAACGAGGACCTGCGCTCCTGGAC | |
| | CGCAGCGGACACTGCGGCTCAGATCTCCAAGCCCAAGTGTGAGGCGGCCAATGTGGCT | |
| | GAACAAAGGAGAGCCTACCTGGAGGGCACGTGCGTGGAGTGGCTCCACAGATACCTCG | |
| | AGAACGGGAAGGAGATGCTGCAGCGCGCGGACCCCCCCAAGACACACGTGACCCACCA | |
| | CCCTGTCTTTGACTATCAGGCCACCCTGAGGTGCTGGGCCCTGGGCTTCTACCCTGCG | |
| | GAGATCATACTGACCTGGCAGCGGGATGGGGAGGACCAGACCCAGGACGTGGAGCTCG | |
| | TGGAGACCAGGCCTGCAGGGGATGGAACCTTCCAGAAGTGGGCAGCTGTGGTGGTGCC | |
| | TTCTGGAGAGGAGCAGAGATACACGTGCCATGTGCAGCATGACGGGCTGCCGGAGCCC | |
| | CTCATGCTGAGATGGGAGCAGTCTTCCCTGCCCACCATCCCCATCATGGGTATCGTTG | |
| | CTCGTCTGGTTGTCCTTGCAGCTGTAGTCACTGGAGCTGCGGTCGCTGCTGTGCTGTG | |
| | GAGGAAGAAGAGCTCAGGTAAGAAAGGAGGGAGCTACTCTCAGGCTGCAAGTAGTGAC | |
| | AGTGCCCAGGGCTCTAATGTGTCTCTCACGGCTTGTAAATGTGACACCCCGGGGGGCC | |
| | TGATGTGTGTGGGTTGTTGAGGGAAACAGTGGACATAGCTGTGCTATGAG | |
| | ORF Start: ATG at 41 | ORF Stop: TGA at 1178 |
| | SEQ ID NO:78 | 379 aa MW at 42027.9 kD |

| | |
|---|---|
| NOV15a, | MVVMAPRTLFLLLSCALTLTETWAGSHSMRYFSAAVSRPGRGEPRFIAMGYVDDTQFV |
| CG59256-01 Protein | RFDSDSACPRMEPRAPWVEQEGPEYWEEETRNTKAHAQTDRMNLQTLRGYYNQSEGVG |
| Sequence | PGSHTLQWMIGCDLGSDGRLLRGYEQYAYDGKDYLALNEDLRSWTAADTAAQISKRKC |
| | EAANVAEQRRAYLEGTCVEWLHRYLENGKEMLQRADPPKTHVTHHPVFDYEATLRCWA |
| | LGFYPAEIILTWQRDGEDQTQDVELVETRPAGDGTFQKWAAVVVPSGEEQRYTCHVQH |
| | EGLPEPLMLRWEQSSLPTIPIMGIVAGLVVLAAVVTGAAVAAVLWRKKSSGKKGGSYS |
| | QAASSDSAQGSNVSLTACKCDTPGGLMCVGC |

Further analysis of the NOV15a protein yielded the following properties shown in Table 15B.

TABLE 15B
Protein Sequence Properties NOV15a

| | |
|---|---|
| PSort | 0.4600 probability located in plasma membrane; 0.1000 |
| analysis: | probability located in endoplasmic reticulum (membrane); |
| | 0.1000 probability located in endoplasmic reticulum (lumen); |
| | 0.1000 probability located in outside |
| SignalP | Cleavage site between residues 25 and 26 |
| analysis: | |

A search of the NOV15a protein against the Geneseq database, a proprietary database that contains sequences published in patents and patent publication, yielded several homologous proteins shown in Table 15C.

TABLE 15C
Geneseq Results for NOV15a

| | | NOV15a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAB36874 | MHC class I protein - Unidentified, 365 | 1 . . . 367 | 299/367 (81%) | e−179 |
| | aa. [US6140305-A, 31-OCT-2000] | 1 . . . 364 | 324/367 (87%) | |
| AAB58683 | HLA-A2/A28 protein #4 - Unidentified, | 1 . . . 367 | 298/367 (81%) | e−179 |
| | 365 aa. [US6153408-A, 28-NOV-2000] | 1 . . . 364 | 324/367 (88%) | |
| AAY52922 | HLA-A2/A28 family peptide A2 (Lee) | 1 . . . 367 | 298/367 (81%) | e−179 |
| | SEQ ID NO: 100 - Mammalia, 365 aa. | 1 . . . 364 | 324/367 (88%) | |
| | [US5976551-A, 02-NOV-1999] | | | |
| AAY68268 | Human leukocyte antigen A2/A28 | 1 . . . 367 | 298/367 (81%) | e−179 |
| | family protein SEQ ID NO: 100 - Homo | 1 . . . 364 | 324/367 (88%) | |
| | sapiens, 365 aa. [US6011146-A, | | | |
| | 04-JAN-2000] | | | |
| AAB58681 | HLA-A2/A28 protein #2 - Unidentified, | 1 . . . 367 | 297/367 (80%) | e−178 |
| | 365 aa. [US6153408-A, 28-NOV-2000] | 1 . . . 364 | 323/367 (87%) | |

In a BLAST search of public sequence databases, the NOV15a protein was found to have homology to the proteins shown in the BLASTP data in Table 15D.

TABLE 15D
Public BLASTP Results for NOV15a

| | | NOV15a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P17693 | HLA class I histocompatibility antigen, | 1 . . . 340 | 334/340 (98%) | 0.0 |
| | alpha chain G precursor (HLA G | 1 . . . 337 | 335/340 (98%) | |
| | antigen) - Homo sapiens (Human), 338 | | | |
| | aa. | | | |
| Q9MYA2 | MHC CLASS I ANTIGEN - Homo | 1 . . . 340 | 332/340 (97%) | 0.0 |
| | sapiens (Human), 338 aa. | 1 . . . 337 | 335/340 (97%) | |
| Q30182 | LYMPHOCYTE ANTIGEN - Homo | 1 . . . 340 | 334/341 (97%) | 0.0 |
| | sapiens (Human), 339 aa. | 1 . . . 338 | 335/341 (97%) | |
| AAH21708 | HYPOTHETICAL 38.3 KDA | 1 . . . 340 | 331/340 (97%) | 0.0 |
| | PROTEIN - Homo sapiens (Human), | 1 . . . 337 | 334/340 (97%) | |
| | 338 aa. | | | |
| Q9TP69 | DJ377H14.1 (MAJOR | 4 . . . 340 | 331/337 (98%) | 0.0 |
| | HISTOCOMPATIBILITY | 1 . . . 334 | 332/337 (98%) | |
| | COMPLEX, CLASS I, G (HLA 6.0)) - | | | |
| | Homo sapiens (Human), 335 aa. | | | |

PFam analysis indicates that the NOV15a protein contains the domains shown in the Table 15E.

TABLE 15E
Domain Analysis of NOV15a

| | | Identities/ | |
|---|---|---|---|
| | NOV15a | Similarities for the | |
| Pfam Domain | Match Region | Matched Region | Expect Value |
| MHC_1: domain | 25 . . . 206 | 145/183 (79%) | 2e−139 |
| 1 of 1 | | 179/183 (98%) | |
| ig: domain 1 of 1 | 223 . . . 288 | 15/67 (22%) | 4.4e−08 |
| | | 45/67 (67%) | |

### Example 16

The NOV16 clone was analyzed, and the nucleotide and polypeptide sequences are shown in Table 16A.

TABLE 16A
NOV16 Sequence Analysis

| | SEQ ID NO:79 | 1175 bp |
|---|---|---|
| NOV16a, | ATTCTCCCCAAACGCCAAGGATGGGGGTCATGGCTCCCCGAACCCTCCTCCTGCTGCT | |
| CG59239-01 DNA | CTTGGGGGCCCTGGCCCTGACCGAGACCTGGGCCGGCTCCCACTCCTTGAGGTATTTC | |
| Sequence | AGCACCGCAGTGTCCCAGCCCGGCCGCGGGGAGCCCCGGTTCATCGCCGTGGGCTACG | |
| | TGGACGACACAGAGTTCGTGCGGTTCGACAGCGACTCCGTGAGTCCGAGCATCCACCG | |
| | GCGGGCGCCGTGGGTGGAGCAGGAGCGGCTGGAGTATTGGGACCAGGAGACACGGAAC | |
| | GCCAAGGGCCACGCGCAGATTTACCGAGTGAACCTGCGGACCCTGCTCCGCTATTACA | |
| | ACCAGAGCGAGCATGGTTCTCACACCATCCAGAGGAAGCATGGCTGCGACGTGGGCCC | |
| | GGACAGGCGCCTCCTCCGCAGGTATGAACAGTTCGCCTACGATGGCAAGGATTACATC | |
| | GCCCTGAACGACGACCTGCACTCCTGGACCCCCGCGAACACAGCGGCTCAGATCTCCC | |
| | AGCACAAGTGGGAAGCGGACAAATACTCAGAGCAGGTCAGGGCCTACCTGCAGCGCAA | |
| | GTGCATGGAGTGGCTCCGCAGACACCTGGAGAACGGGAAGGAGACGCTGCAGCACGCG | |
| | GATCCCCCAAAGGCACATGTGACCCAGCACCCCATCTCTGACCATGAGGCCACCCTGA | |
| | GGTGCTGGGCCCTGGGCCTCTACCCTGCGGAGATCACACTGACCTGGCAGCAGGATGG | |
| | GGAGGACCAGACCCACGACACGGAGCTTGTGGAGACCAGGCCTGCAGGGGACGGAACC | |
| | GCCCACCATCCCCATCGTGGGCATCGTTGCTGGCCTGTTTCTCCTTGGAGCTGTGGTC | |
| | ACTGCAGCTGTGGTTGCTGCTGCOATOTGGAGCAAGAAAAGCTCAGGTAGGGAAGGCG | |
| | TGAGAGGTTCTACCCCAGGCAGCAATTGTGCTCAGTACTCTGATGCATCTCATGATAC | |
| | TTGTAAAGCTTGAGACAACTGCCTTGAGTGGGACTGAGAGATACAAAATTTCTTCAGG | |
| | TCCTTCCTCTGACAC | |
| | ORF Start: ATG at 21 | ORF Stop: TGA at 1113 |
| | SEQ ID NO:80 | 364 aa MW at 40983.6 kD |

| | |
|---|---|
| NOV16a, | MGVMAPRTLLLLLLGALALTETWAGSHSLRYFSTAVSQPGRGEPRFIAVGYVDDTEFV |
| CG59239-01 Protein | RFDSDSVSPRMERRAPWVEQEGLEYWDQETRNAKGHAQIYRVNLRTLLRYYNQSEHGS |
| Sequence | HTIQRKHGCDVGPDRRLLRRYEQFAYDGKDYIALNEDLHSWTAANTAAQISQHKWEAD |
| | KYSEQVRAYLEGKCMEWLRRHLENGKETLQHADPPKAHVTQHPISDHEATLRCWALGL |
| | YPAEITLTWQQDGEDQTQDTELVETRPAGDGTFQKWVAVVVPSGEEQRYMCHVQHEGL |
| | PEPLTLRWGPSSQPTIPIVGIVAGLFLLGAVVTGAVVAAAMWRKKSSGREGVRGSTPG |
| | SNCAQYSDASHDTCKA |

Further analysis of the NOV16a protein yielded the following properties shown in Table 16B.

TABLE 16B
Protein Sequence Properties NOV16a

| | |
|---|---|
| PSort | 0.4600 probability located in plasma membrane; 0.1357 |
| analysis: | probability located in microbody (peroxisome); 0.1000 |
| | probability located in endoplasmic reticulum (membrane); |
| | 0.1000 probability located in endoplasmic reticulum (lumen) |
| SignalP | Cleavage site between residues 25 and 26 |
| analysis: | |

A search of the NOV16a protein against the Geneseq database, a proprietary database that contains sequences published in patents and patent publication, yielded several homologous proteins shown in Table 16C.

TABLE 16C
Geneseq Results for NOV16a

| | | NOV16a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAM24017 | Human EST encoded protein SEQ ID | 4 . . . 363 | 276/367 (75%) | e−165 |
| | NO: 1542 - Homo sapiens, 368 aa. | 1 . . . 367 | 307/367 (83%) | |
| | [WO200154477-A2, 02-AUG-2001] | | | |
| AAB58681 | HLA-A2/A28 protein #2 - | 1 . . . 363 | 274/364 (75%) | e−164 |
| | Unidentified, 365 aa. [US6153408-A, | 1 . . . 364 | 305/364 (83%) | |
| | 28-NOV-2000] | | | |
| AAB58680 | HLA-A2/A28 protein #1 - | 1 . . . 363 | 274/364 (75%) | e−164 |
| | Unidentified, 365 aa. [US6153408-A, | 1 . . . 364 | 305/364 (83%) | |
| | 28-NOV-2000] | | | |
| AAY52920 | HLA-A2/A28 family peptide A2.4a | 1 . . . 363 | 274/364 (75%) | e−164 |
| | SEQ ID NO: 98 - Mammalia, 365 aa. | 1 . . . 364 | 305/364 (83%) | |
| | [US5976551-A, 02-NOV-1999] | | | |
| AAY52919 | HLA-A2/A28 family peptide HLA- | 1 . . . 363 | 274/364 (75%) | e−164 |
| | A2.1 SEQ ID NO: 97 - Mammalia, | 1 . . . 364 | 305/364 (83%) | |
| | 365 aa. [US5976551-A, | | | |
| | 02-NOV-1999] | | | |

In a BLAST search of public sequence databases, the NOV16a protein was found to have homology to the proteins shown in the BLASTP data in Table 16D.

TABLE 16D
Public BLASTP Results for NOV16a

| | | NOV16a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9TQP7 | MHC CLASS I ANTIGEN - Homo | 1 . . . 363 | 286/370 (77%) | e−168 |
| | sapiens (Human), 371 aa. | 1 . . . 370 | 312/370 (84%) | |
| Q30718 | MHC CLASS I ANTIGEN | 1 . . . 363 | 282/364 (77%) | e−167 |
| | MAMUA*07 - Macaca mulatta | 1 . . . 364 | 312/364 (85%) | |
| | (Rhesus macaque), 365 aa. | | | |
| Q95J07 | MHC CLASS I ANTIGEN HEAVY | 1 . . . 363 | 286/366 (78%) | e−167 |
| | CHAIN - Homo sapiens (Human), | 1 . . . 364 | 310/366 (84%) | |
| | 365 aa. | | | |
| Q9MYI5 | HLA CLASS I ANTIGEN - Homo | 1 . . . 363 | 286/366 (78%) | e−167 |
| | sapiens (Human), 365 aa. | 1 . . . 364 | 311/366 (84%) | |
| Q9TQP6 | sapiens (Human), 365 aa. | 1 . . . 364 | 311/366 (84%) | e−167 |

PFam analysis indicates that the NOV16a protein contains the domains shown in the Table 16E.

TABLE 16E
Domain Analysis of NOV16a

| | NOV16a | Identities/ | |
|---|---|---|---|
| | Match | Similarities for | Expect |
| Pfam Domain | Region | the Matched Region | Value |
| MHC_I: domain 1 of 1 | 25 . . . 203 | 139/180 (77%) | 5.6e−131 |
| | | 165/180 (92%) | |
| ig: domain 1 of 1 | 220 . . . 285 | 12/67 (18%) | 9.9e−08 |
| | | 47/67 (70%) | |

### Example 17

The NOV17 clone was analyzed, and the nucleotide and polypeptide sequences are shown in Table 17A.

TABLE 17A
NOV17 Sequence Analysis

| | SEQ ID NO:81 | 8838 bp |
|---|---|---|
| NOV17a, | AGCAGAGCCCTGGGCCAGGCTCTCTGATTGCCTGGCACCCTGATGGGAGCCCGCATGC | |
| CG59295-01 DNA | CCAGACGATGCTTGCTTCTTCTCTCTTGCTTTTGTCTACTTAGGGTTGAGTCCACTGC | |
| Sequence | AGAGGTGCAGCACCAGGCCTCAGCACTCACATGGAAAATCAGTGCAGAGCTGCAACAG | |
| | GAGCCTGCTCCTGAGCCCAGCCACACATACCAGGAGATGTCCCTTGCAGTGGAGGATG | |
| | TCACAACAGTTATGGAAGGCAAGCAGGCTGAAGCCCCAGACTCCGTGGCCATGTCTTC | |
| | CTGGGAAAGGCGGCTCCATCGGGCCAAGTGTGCACCATCCTACTTGTTCTCCTGCTTC | |
| | AATCGAGGCGAGTGTGTGCACCCAGCCTTCTGTCACTGCAGACGCTTCAATCCCACTG | |
| | GACCGCGCTGCCAGATGGTGTACAATGCCGGCCCTGAGAGGGACAGCATTTGCCGGGC | |
| | GTGGGGGCAGCACCACGTGGAGACATTTGATGGGCTCTACTACTACCTCTCCGGAAAG | |
| | GGCAGCTACACCCTGGTGGGTCGCCATGAGCCCGAGGGACAGAGCTTCTCCATCCAGG | |
| | TACACAATGACCCGCAGTGTGGCTCTTCACCCTACACCTGCTCCAGGGCTGTCAGCCT | |
| | CTTCTTTGTGGGTGAGCAGGAGATCCATCTGGCCAAGGAGGTCACCCATGGAGGCATG | |
| | AGGGTCCAACTGCCACATGTCATGGGCAGCGCGCGTCTGCAGCAGCTTGCCGGCTATG | |
| | TCATCGTGCGGCATCAGTCAGCCTTCACACTGGCCTGGGATGGTGCCTCGGCTGTCTA | |
| | CATCAAGATGAGTCCAGAGCTTCTGGGCTGGACCCATGGGCTGTGTGGGAACAACAAT | |
| | GCTGACCCCAAGGATGATCTGGTGACCAGCTCTGGTGAGGGGAAGCTGACTGACGACG | |
| | TGGTTGAGTTTGTGCACAGCTGGCAGGAGCAGGCCCCTAACCAGCCTCCAGGGCCCAC | |
| | AACTTCCTCCCTGCCTCGCCCACCGTGCCTACAGCAGAACCCAGGAACCATGCAGGGC | |
| | GTGTACGAGCACTGTGACGCTCTACTGCGGCCCCCCTTTGACCCCTGCCACGCCTACG | |
| | TCAGCCCTCTGCCCTTCACAGCCAGTTGTACCAGTGATCTCTGCCAATCAATGGGTGA | |
| | TGTAGCCACCTGGTGCCGGGCACTGGCGGAGTATGCCCGGGCGTGTGCCCAGGCAGGG | |
| | CGGCCCTTGCAAGGCTGGAGGACCCAGCTCCGGCAATGCACTGTGCACTGCAAGGAGA | |
| | AGGCCTTTACCTACAATGAGTGCATCGCCTGCTGCCCTGCCTCCTGCCATCCCCGGGC | |
| | ATCCTGTGTGGACAGTGAGATCGCCTGTGTGGACGGCTGCTATTGCCCCAATGGGCTC | |
| | ATCTTCGAGGATGGGGGCTGCGTGGCACCAGCTGAGTGTCCCTGTGAGTTTCACGGGA | |
| | CTCTGTACCCACCTGGCTCTGTGGTGAAGGAAGACTGCAATACTTGCACATGCACCTC | |
| | AGGCAAGTGGGACTGCAGCACACCTGTCTGCCCAGCTGAGTGCTCAGTGACTGGTGAC | |
| | ATTCACTTCACAACCTTTGATGGCCGCCGGTACACGTTCCCCGCCACATGTCAGTACA | |
| | TCCTGGCCAAGAGCCGCTCTTCGGCCACCTTCACCGTGACATTGCAGAATGCCCCATG | |
| | TGGCCTGAACCAAGATGGAGCCTGTGTCCAGTCAGTGTCAGTGATTCTGCACCAGGAC | |
| | CCTCGGAGGCAGGTGACCCTGACCCAGGCAGGGGATGTCCTTCTGTTTCACCAGTACA | |
| | AGATCATCCCGCCATACACAGATGATGCCTTTGAGATCCGTAGGCTGTCCTCCGTGTT | |
| | CCTGCGGGTGAGGACGAACGTGGGCGTGCGGGTGCTCTACGACCGTGAAGGGCTCCGA | |
| | CTGTACCTGCAAGTGGACCAGCGATGGGTGGAGGATACCGTGGGCCTCTGCCGCACCT | |
| | TCAATGGCAACACGCAGGATGACTTCCTGTCTCCAGTGGGTGTACCTGAGAGCACCCC | |
| | ACAACTTTTTGGCAATTCCTGGAAAACACTTTCTGCTTGCTCCCCGCTGGTCTCTGGC | |
| | TCCCCTCTGGACCCCTGCGATCTGCACCTGCAAGCCGCCTCCTACTCAGTGCAGGCCT | |
| | GCAGCGTGCTCACGGGGGAGATGTTTGCGCCCTGCTCTGCGTTCCTGAGCCCCGTGCC | |
| | CTACTTTCAGCAGTGCCGCAGCGATGCCTGCCGCTGCGGGCAGCCCTGCCTGTGCGCC | |
| | ACACTGGCCCACTACGCCCACCTGTGCCGGCGCCATGGGCTCCCCGTTGATTTCCGCG | |
| | CCCGCCTGCCAGCCTGTGCACTGTCCTGTGAGGCCTCCAAGGAGTATAGCCCCTGCCT | |
| | GGCCCCGTGTGGACGTACCTGCCAGGACCTGGCCAGCCCTGAGGCCTGTGGGGTTGAT | |
| | GGTGGCGATGACCTGAGCAGAGACGAGTGTGTGGAGGGCTGTGCCTGCCCACCGGACA | |
| | CCTATCTGGACACCCAGGCTGACCTCTGTGTCCCCCGGAACCAGTGCTCCTGCCACTT | |
| | CCAGGGAGTGGACTATCCCCCCGGAGACAGTGACATCCCATCCCTGGGCCACTGCCAC | |
| | GCCAGGTCTTCGTGAACTGCAGCGACCTGCACACGGACCTGGAGCTGAGCAGGGAGAG | |
| | GACGTGTGAGCAGCAACTGCTGAACCTGAGCGTGTCAGCCCGTGGCCCCTGCCTCTCG | |
| | GGCTGCGCCTGTCCCCAGGGTCTGCTCAGACACGGGGATCCATGTTTCCTGCCAGAGG | |
| | AGTGCCCCTGCACTTGGAAGGGGAAGGAGTATTTCCCTGGGGACCAGGTGATGTCTCC | |
| | TTGCCATACCTGTGTGTGCCAGCGGGGCTCATTCCAGTGCACCCTGCACCCTTGCGCC | |
| | TCCACCTGCACTGCCTATGGGGACCGGCATTACCGCACGTTTGATGGGCTCCCGTTTG | |
| | ACTTCGTGGGGGCATGCAAAGTGCACCTGGTCAAGAGCACATCAGATGTCAGCTTCTC | |
| | TGTGATTGTAGAGAATGTGAACTGCTACAGCTCTGGCATGATCTGCAGGAAATTTATT | |
| | TCCATCAACGTTGGGAACTCACTCATTGTCTTTGATGATGACTCCGCAAATCCTAGTC | |
| | CAGAGAGCTTCCTGGATGACAAGCAGGAGGTCCACACATGGCGAGTGGGATTTTTCAC | |
| | ACTGGTGCATTTCCCACAGGAGCACATCACCCTCTTGTGGGACCAGAGAACCACAGTG | |
| | CACGTCCAGGCTGGGCCTCAGTGGCAGGGCCACCTGGCCGGCCTCTGTCGGAACTTTG | |
| | ACTTAAAAACCATCAATGAGATGAGGACCCCGGAGAACCTAGAGCTAACTAACCCCCA | |
| | GGAGTTTGGCAGCAGTTGGGCTGCAGTTGAGTGCCCAGACACCCTCGATCCTCGGGAT | |
| | ATGTGTGTCCTGAATCCTCTCCCACAACCATTTGCCAAGAAGGAGTGCAGCATCCTGC | |
| | TCAGTGAGGTGTTTGAGATCTGCCACCCTGTGGTTGATGTCACTTGGTTTTACTCAAA | |
| | CTGCCTGACAGACACATGTGGCTGCAGCCAGGGTGGTGACTGTGAGTGCTTCTGTGCC | |
| | AGCGTCTCCGCTTATGCCCACCAGTGTTGCCAGCATGGGGTGGCTGTTGACTGGCGAA | |
| | CCCCCCGCCTCTGCCCGTATGACTGTGACTTCTTTAACAAAGTGCTAGGTAAGGGCCC | |
| | CTATCAGCTATCCAGCTTGGCAGCCGGTGGTGCTCTGGTGGGCATGAAGGCGGTGGGC | |
| | GATCACATAGTCCTAGTGAGGACAGAGGATGTGGCGCCAGCAGACATTGTGAGCTTCC | |
| | TCCTGACAGCTGCTCTGTACAAGGCCAAGGCCCATGACCCAGATGTGGTGTCCCTGCA | |
| | GGCAGCAGACAGACCCAACTTCTTCCTTCACGTCACAGCCAACGGGTCTCTGGAGCTG | |
| | GCTAAGTGGCAGGGCCGTGACACCTTCCAACAGCATGCCTCCTTCTTGCTGCACCGGG | |
| | GGACACCGCAGGCAGGCCTCGTGGCCCTGGAGTCCCTGGCCAAGCCCAGCTCCTTCCT | |
| | CTATGTGTCGGGCGCCGTGCTGGCCCTGCGGCTGTACGAACACACAGAGGTGTTCCGC | |
| | CGGGGCACACTCTTCCGCCTTCTGGATGCCAAGCCCTCGGGGGCTGCCTACCCCATCT | |
| | GCGAGTGGCGCTACGATGCCTGTGCCAGCCCCTGCTTCCAAACCTGCCGGGACCCACG | |
| | GGCAGCCAGCTGCCGGGACGTACCCAGGGTAGAAGGCTGTGTCCCTGTGTGCCCCACC | |
| | CCCCAGGTCCTGGATGAAGTCACACAGAGATGTGTCTACTTGGAGGACTGTGTGGAGC | |
| | CAGCACTTTCGGTTCCCACAGAGGCCCTTGGCAATGAGACCCTCCCTCCCAGTCAAGG | |
| | GTTGCCCACTCCCAGTGATGAGGAGCCACAGCTGTCACAGGAAAGCCCCAGGACCCCC | |
| | ACCCACAGGCCAGCCCTCACCCCAGCTGCCCCACTCACCACAGCCCTGAACCCACCAG | |
| | TGACAGCCACTGAGGAGCCAGTGGTGTCTCCAGGCCCCACCCAGACCACCCTGCAGCA | |
| | GCCACTGGAGCTCACTGCATCTCAACTCCCCGCCGGCCCCACGGAGTCCCCAGCCAGC | |
| | AAGGGAGTGACTGCCAGCCTCCTGGCCATCCCCCATACACCAGAGTCCTCATCCCTCC | |
| | CTGTTGCACTGCAGACACCCACACCTGGCATGGTGTCAGGTGCCATGGAGACAACAAG | |
| | GGTGACTGTGATCTTTGCAGGAAGCCCTAACATCACAGTCTCCTCCCGGTCGCCCCCT | |
| | GCCCCTCGCTTCCCGCTCATGACCAAGGCTGTGACAGTCCGAGGCCATGGCTCCTTGC | |
| | CTGTTAGGACGACACCCCCACAGCCCTCCTTGACAGCAAGTCCCTCCTCCAGACCTGT | |
| | GGCTTCCCCTGGAGCCATCTCCAGGTCCCCCACCTCCTCGGGATCCCACAAGGCTGTG | |
| | CTGACACCTGCAGTAACTAAGGTCATAAGCAGGACAGGGGTCCCCCAGCCCACCCAGG | |
| | CCCAGAGTGCTTCAAGTCCCAGCACCCCTCTAACTGTGGCTGGAACAGCAGCAGAACA | |
| | GGTTCCTGTCAGTCCCCTTGCAACCAGGAGCTTGGAGATAGTGCTATCCACAGAGAAG | |
| | GGCGAAGCCGGGCACAGCCAGCCCATGGGCTCGCCTGCCTCCCCACAGCCACACCCAC | |
| | TCCCCTCTGCACCACCCCCCCCAGCCCAGCATACCACCATGGCCACCAGGTCTCCAGC | |
| | TCTGCCCCCACACACCCCAGCTGCCGCCAGCCTGTCAACAGCCACTGATGGGCTGGCA | |
| | GCCACACCCTTCATGTCCCTTGAGTCAACTCGTCCCTCCCAGCTCCTCTCTGGCCTGC | |
| | CTCCCGACACCAGCCTGCCCCTGGCCAAGGTGGGCACATCTGCCCCAGTGGCCACACC | |
| | CGGCCCCAAAGCCTCTGTCATCACCACTCCACTCCACCCACAGGCCACCACTCTGCCT | |
| | GCTCAGACACTTAGCCCAGTACTGCCTTTCACTCCAGCAGCAATGACCCAGGCGCACC | |
| | CACCCACTCACATAGCACCCCCAGCAGCAGGCACAGCTCCAGGCCTGCTGCTGGGAGC | |
| | CACATTGCCAACCTCTGGAGTCCTGCCTGTGGCTGAGGGCACGGCCTCCATGGTATCT | |
| | GTTGTCCCACGAAAGAGCACCACAGGGAAGGTGGCCATCCTATCCAAGCAAGTGTCTC | |
| | TGCCCACTTCCATGTATGGTTCTGCAGAGGGTGGGCCCACAGAGCTCACCCCTGCTAC | |
| | GAGCCACCCTCTCACGCCCTTGGTGGCTGAGCCCGAGGGAGCCCAGGCAGGCACAGCT | |
| | CTGCCAGTGCCCACATCCTATGCCCTGAGCCGTGTCTCAGCCAGGACGGCCCCCCAAG | |
| | ACAGCATGCTGGTTCTGTTGCCTCAGCTGGCTGAGGCCCATGGAACCTCGGCAGGGCC | |
| | TCACCTGGCAGCAGACCCGGTGGACGAGGCCACCACAGAACCATCTGGGCGCTCAGCC | |
| | CCAGCCCTGAGCATCGTAGAGGGTTTGGCGGAGGCTTTGGCAACTACCACTGAGGCCA | |
| | ATACATCCACCACCTGTGTTCCAATCGCCGAGCAGGACTGCGTCCGCCACATCTGCCT | |
| | GGAGGGCCAGCTGATTCGCGTGAATCAGTCCCAGCACTGTCCCCAGGGTGCTGCTCCC | |
| | CCTCGCTGTGGGATCCTGGGCCTCGCCCTGCGGGTGGGTGGGGACCGCTGCTGCCCAC | |
| | TCTGGGAGTGTGCCTGCCGGTGCTCAATCTTCCCTGACCTGAGCTTCGTGACCTTCGA | |
| | TGGGAGCCACGTAGCTCTGTTCAAGGAGGCCATCTACATCCTCAGCCAGAGCCCAGAT | |
| | GAAATGCTCACCGTCCATGTACTGGACTGCAAAAGTGCCAACCTCGGGCACCTGAACT | |
| | GGCCCCCGTTCTGTCTGGTGATGTTGAACATGACTCACTTGGCCCATCAGGTCACTAT | |
| | TGATCGCTTCAACCGAAAGGTGACTGTGGACTTGCAGCCTGTGTGGCCACCGGTGAGC | |
| | AGGTATGCATTCAGAATTGAGGACACAGGCCACATGTACATGATCCTGACTCCCTCAG | |
| | ACATCCAGATCCAGTGGCTCCACAGCTCAGGACTCATGATCGTGGAGGCCAGCAAAAC | |
| | CAGCAAGGCCCAGGGCCATGGCCTGTGCGGTATCTGTGATGGAGATGCAGCCAATGAC | |
| | CTTACCCTGAAGGATGGCTCAGTGGTGCGTGCGGCTGAGGACCCTGCTCCCTTTCTGG | |
| | ACAGCTGGCAGGTGCCCAGCTCCCTGACCTCAGTGGGCCAGACCCGCTTCCGCCCAGA | |
| | CAGCTGCGCCACAACTGACTGCTCGCCCTGCCTTCGCATGGTGTCCAACCGCACCTTC | |
| | AGTGCCTGCCACCGCTTTGTGCCTCCGGAGTCATTCTGTGAGCTGTGGATCCGGGACA | |
| | CCAAGTACGTGCAGCAGCCCTGCGTGGCCCTGACTGTGTACGTGGCCATGTGCCACAA | |
| | ATTTCATGTGTGCATCGAGTGGCGGCGCTCTGACTACTGCCCCTTCCTGTGCTCCAGC | |
| | GACTCCACATACCAGGCATGTGTGACAGCCTGTGAGCCACCCAAGACATGCCAGGATG | |
| | GGATACTAGGGCCTCTGGACCCAGAGCACTGCCAGGTGCTGGGCGAGGGCTGCGTCTG | |
| | CTCCGAGGGCACCATCTTACACCGGCGCCACTCTGCACTCTGCATCCCGGAGGCCAAG | |
| | TGCGCCTGCACTGACAGCATGGGGGTGCCGAGGGCCCTGGGGGAGACCTGGAACAGCT | |
| | CCCTCAGCGGCTGCTGCCAGCACCAGTGCCAAGCCCCAGACACCATTGTCCCGGTGGA | |
| | TCTGGGCTGCCCCAGTCCCCGCCCTGAGAGCTGCCTGCGATTCGGCGAGGTGGCCTTG | |
| | CTCCTACCCACCAAGGACCCCTGCTGCCTGGGGACTGTCTGTGTGTGTAACCAGACTC | |
| | TGTGTGAGGGTCTCGCCCCCACATGCCGCCCAGGCCACCGCCTCCTCACCCACTTCCA | |
| | GGAGGACTCCTGCTGCCCCAGCTACAGCTGTGAGTGTGACCCAGATCTCTGTGAGGCA | |
| | GAGCTGGTCCCCAGCTGCCGACAGGACCAGATCCTGATCACGGGCCGCCTGGGGGACT | |
| | CCTGCTGCACCTCCTACTTCTGCGCCTGTGGTGACTGTCCAGACTCCATCCCCGAATG | |
| | TCAAGAAGGGGAGGCGCTCACTGTGCACAGGAATACCACGGAACTCTGCTCCCCTCTG | |
| | TACCAGTGTGTGTGTGAGAACTTCCGCTGTCCCCAAGTGCAGTGTGCCCTGGGCACTG | |
| | CCCTGGTCGAGGTGTGGAGCCCCGACCGCTGCTGCCCCTACAAATCCTGTGAATGTGA | |
| | CTGTGACACAATCCCGGTGCCCCGGTGCCATCTGTGGGAGAAATCCCAGCTGGATGAG | |
| | GAGTTCATGCACAGCGTGGAGAATGTGTGTGGCTGCGCCAAGTACGAGTGTGTGAAGG | |
| | CCCCGGTGTGTCTGAGCCGCGAGCTGGGTGTGATGCAGCCCGGCCAGACAGTGGTGCA | |
| | GCTCTCAGCAGATGGCCTGTGCCACACCTCCCGCTGCACCACCGTGCTCGACCCTCTC | |
| | ACCAACTTCTACCAGATCAACACCACCTCCGTGCTCTGTGACATCCACTGTGAGGCGA | |
| | ACCAGGAGTACGAGCACCCGCGGGACCTCGCTGCCTGCTGCGGCTCCTGCAGGAACGT | |
| | GTCCTGTCTCTTCACCTTCCCCAATGGCACCACCTCCCTGTTCTTGCCCGGGCCATCC | |
| | TGGATCGCAGACTGCGCCCGCCACCACTGCAGCAGCACGCCCCTGGGTGCCGTGCTGG | |
| | TCCGCTCTCCCATAAGCTGCCCACCGCTCAATGAGACTGAGTGTGCCAAGGTTGGGGG | |
| | TTCCGTGGTACCTTCCTTGGAAGGATGCTGCAGGACCTGTAAGGAGGATGGGCGCTCC | |
| | TGCAAGAAGGTGACCATCCGCATGACCATCCGCAAGAATGAATGCAGGAGCAGCACCC | |
| | CTGTGAACCTAGTGTCCTGCGATGGGAGGTGCCCATCCGCCAGCATCTACAACTACAA | |
| | CATCAACACCTATGCCCGATTCTGCAAGTGCTGCCGTGAGGTGGGCCTGCAGCGGCGC | |
| | TCTGTGCAGCTCTTCTGTGCCACCAATGCCACCTGGGTGCCCTATACAGTGCACGAGC | |
| | CCACCGACTGTGCCTGCCAGTGGTCCTGAGGCCTGGGGCCCCCGGCTAGCTGGACCAC | |
| | CTCTGCCAGCCCCACTTTCTGT | |
| | ORF Start: ATG at 43 | ORF Stop: TGA at 8785 |
| | SEQ ID NO:82 | 2914 aa MW at 314011.9 kD |

| | |
|---|---|
| NOV17a, | MGARMPRRCLLLLSCFCLLRVESTAEVQHQASALTWKTSAELQQEPAPEPSHTYQEMS |
| CG59295-01 Protein | LAVEDVTTVMEGKQAEAPDSVAMSSWERRLHRAKCAPSYLFSCFNGGECVHPAFCDCR |
| Sequence | RFNATGPRCQMVYNAGPERDSICRAWGQHHVETFDGLYYYLSGKGSYTLVGRHEPEGQ |
| | SFSIQVHNDPQCGSSPYTCSRAVSLFFVGEQEIHLAKEVTHGGMRVQLPHVNGSARLQ |
| | QLAGYVIVRHQSAFTLAWDGASAVYIKMSPELLGWTHGLCGNNNADPKDDLVTSSGEG |
| | KLTDDVVEFVHSWQEQAPNQPPGPTTSSLPRPPCLQQNPGTMQGVYEQCEALLRPPFD |
| | ACHAYVSPLPFTASCTSDLCQSMGDVATWCRALAEYARACAQAGRPLQGWRTQLRQCT |
| | VHCKEKAFTYNECIACCPASCHPRASCVDSEIACVDGCYCPNGLIFEDGGCVAPAECP |
| | CEFHGTLYPPGSVVKEDCNTCTCTSGKWECSTAVCPAECSVTGDIHFTTFDGRRYTFP |
| | ATCQYILAKSRSSGTFTVTLQNAPCGLNQDGACVQSVSVILHQDPRRQVTLTQAGDVL |
| | LFDQYKIIPPYTDDAFEIRRLSSVFLRVRTNVGVRVLYDREGLRLYLQVDQRWVEDTV |
| | GLCCTFNGNTQDDFLSPVGVPESTPQLFGNSWKTLSACSPLVSGSPLDPCDVHLQAAS |
| | YSVQACSVLTGEMFAPCSAFLSPVPYFEQCRRDACRCGQPCLCATLAHYAHLCRRHGL |
| | PVDFRARLPACALSCEASKEYSPCVAPCGRTCQDLASPEACGVDGGDDLSRDECVEGC |
| | ACPPDTYLDTQADLCVPRNQCSCHFQGVDYPPGDSDIPSLCHCHCKDGVMSCDSRAPA |
| | AACPAGQVFVNCSDLHTDLELSRERTCEQQLLNLSVSARGPCLSGCACPQGLLRHCDA |
| | CFLPEECPCTWKGKEYFPGDQVMSPCHTCVCQRGSFQCTLHPCASTCTAYGDRHYRTF |
| | DGLPFDFVGACKVHLVKSTSDVSFSVIVENVNCYSSGMICRKFISINVGNSLIVFDDD |
| | SGNPSPESFLDDKQEVHTWRVGFFTLVHFPQEHITLLWDQRTTVHVQAGPQWQGQLAG |
| | LCGNFDLKTINEMRTPENLELTNPQEFGSSWAAVECPDTLDPRDMCVLNPLREPFAKK |
| | ECSILLSEVFEICHPVVDVTWFYSNCLTDTCGCSQGGDCECFCASVSAYAHQCCQHGV |
| | AVDWRTPRLCPYDCDFFNKVLGKGPYQLSSLIAAGGALVGMKAVGDDIVLVRTEDVAPA |
| | DIVSFLLTAALYKAKAHDPDVVSLEAADRPNFFLHVTANIGSLELAKWQGRDTFQQHAS |
| | FLLHRGTRQAGLVALESLAKPSSFLYVSCAVLALRLYEHTEVFRRGTLFRLLDAKPSG |
| | AAYPICEWRYDACASPCFQTCRDPRAASCRDVPRVEGCVPVCPTPQVLDEVTQRCVYL |
| | EDCVEPAVWVPTEALGNETLPPSQGLPTPSDEEPQLSQESPRTPTHRPALTPAAPLTT |
| | ALNPPVTATEEPVVSPGPTQTTLQQPLELTASQLPAGPTESPASKGVTASLLAIPHTP |
| | ESSSLPVALQTPTPGMVSGAMETTRVTVIFAGSPNITVSSRSPPAPRFPLMTKAVTVR |
| | GHGSLPVRTTPPQPSLTASPSSRPVASPGAISRSPTSSGSHKAVLTPAVTKVISRTGV |
| | PQPTQAQSASSPSTPLTVAGTAAEQVPVSPLATRSLEIVLSTEKGEAGHSQPMGSPAS |
| | PQPHPLPSAPPRPAQHTTMATRSPALPPETPAAASLSTATDGLAATPFMSLESTRPSQ |
| | LLSGLPPDTSLPLAKVGTSAPVATPGPKASVITTPLQPQATTLPAQTLSPVLPFTPAA |
| | MTQAHPPTHIAPPAAGTAPGLLLGATLPTSGVLPVAEGTASMVSVVPRKSTTGKVAIL |
| | SKQVSLPTSMYGSAEGGPTELTPATSHPLTPLVAEPEGAQAGTALPVPTSYALSRVSA |
| | RTAPQDSMLVLLPQLAEAHGTSAGPHLAAEPVDEATTEPSGRSAPALSIVEGLAEALA |
| | TTTEANTSTTCVPIAEQDCVRHICLEGQLIRVNQSQHCPQGAAPPRCGILGLAVRVGG |
| | DRCCPLWECACRCSIFPDLSFVTFDGSHVALFKEAIYILSQSPDEMLTVHVLDCKSAN |
| | LGHLNWPPFCLVMLNNTHLAHQVTIDRFNRKVTVDLQPVWPPVSRYGFRIEDTGHMYM |
| | ILTPSDIQIQWLHSSGLMIVEASKTSKAQGHGLCGICDGDAANDLTLKDGSVVGGAED |
| | PAPFLDSWQVPSSLTSVGQTRFRPDSCATTDCSPCLRMVSNRTFSACHRFVPPESFCE |
| | LWIRDTKYVQQPCVALTVYVANCHKFHVCIEWRRSDYCPFLCSSDSTYQACVTACEPP |
| | KTCQDGILGPLDPEHCQVLGEGCVCSEGTILHRRHSALCIPEAKCACTDSMGVPRALG |
| | ETWNSSLSGCCQHQCQAPDTIVPVDLGCPSPRPESCLRFGEVALLLPTKDPCCLGTVC |
| | VCNQTLCEGLAPTCRPGHRLLTHFQEDSCCPSYSCECDPDLCEAELVPSCRQDQILIT |
| | GRLGDSCCTSYFCACGDCPDSIPECQEGEALTVHRNTTELCCPLYQCVCENFRCPQVQ |
| | CGLGTALVEVWSPDRCCPYKSCECDCDTIPVPRCHLWEKSQLDEEFMHSVENVCGCAK |
| | YECVKAPVCLSRELGVMQPGQTVVELSADGVCHTSRCTTVLDPLTNFYQINTTSVLCD |
| | IHCEANQEYEHPRDLAACCGSCRNVSCLFTFPNGTTSLFLPGASWIADCARHHCSSTP |
| | LGAVLVRSPISCPPLNETECAKVCGSVVPSLEGCCRTCKEDGRSCKKVTIRMTIRKNE |
| | CRSSTPVNLVSCDGRCPSASIYNYNINTYARFCKCCREVGLQRRSVQLFCATNATWVP |
| | YTVQEPTDCACQWS |

Further analysis of the NOV17a protein yielded the following properties shown in Table 17B.

TABLE 17B
Protein Sequence Properties NOV17a

| | |
|---|---|
| PSort | 0.4610 probability located in outside; 0.1900 |
| analysis: | probability located in lysosome (lumen); 0.1000 |
| | probability located in endoplasmic reticulum (membrane); |
| | 0.1000 probability located in endoplasmic reticulum |
| | (lumen) |
| SignalP | Cleavage site between residues 24 and 25 |
| analysis: | |

A search of the NOV17a protein against the Geneseq database, a proprietary database that contains sequences published in patents and patent publication, yielded several homologous proteins shown in Table 17C.

TABLE 17C
Geneseq Results for NOV17a

| | | | | |
|---|---|---|---|---|
| | | NOV17a | Identities/ | |
| | | Residues/ | Similarities for | |
| Geneseq | Protein/Organism/Length | Match | the Matched | Expect |
| Identifier | [Patent #, Date] | Residues | Region | Value |

| | | | | |
|---|---|---|---|---|
| AAU29530 | Novel human secreted protein #21 - | 139 . . . 1233 | 392/1136 (34%) | 0.0 |
| | Homo sapiens, 2814 aa. | 35 . . . 1131 | 561/1136 (48%) | |
| | [WO200179449-A2, 25-OCT-2001] | | | |
| AAP60053 | Sequence of von Willebrand factor | 139 . . . 1233 | 392/1136 (34%) | 0.0 |
| | (vWF) - Homo sapiens, 2813 aa. | 35 . . . 1131 | 561/1136 (48%) | |
| | [EP197592-A, 15-OCT-1986] | | | |
| AAP60462 | Sequence of human von Willebrand | 139 . . . 1233 | 393/1137 (34%) | 0.0 |
| | Factor (VWF) precursor - Homo | 35 . . . 1131 | 559/1137 (48%) | |
| | sapiens, 2813 aa. [WO8606096-A, | | | |
| | 23-OCT-1986] | | | |
| AAY70557 | Canine von Willebrand factor - Canis | 139 . . . 1233 | 386/1137 (33%) | e−179 |
| | familiaris, 2813 aa. [WO200009533-A1, | 35 . . . 1131 | 550/1137 (47%) | |
| | 24-FEB-2000] | | | |
| AAW54347 | Canine von Willebrand Factor (vWF) | 139 . . . 1233 | 386/1137 (33%) | e−179 |
| | sequence - Canis sp, 2813 aa. | 35 . . . 1131 | 550/1137 (47%) | |
| | [WO9803683-A1, 29-JAN-1998] | | | |

In a BLAST search of public sequence databases, the NOV17a protein was found to have homology to the proteins shown in the BLASTP data in Table 17D.

TABLE 17D
Public BLASTP Results for NOV17a

| | | NOV17a | Identities/ | |
|---|---|---|---|---|
| Protein | | Residues/ | Similarities | |
| Accession | | Match | for the | Expect |
| Number | Protein/Organism/Length | Residues | Matched Portion | Value |
| O55225 | OTOGELIN - Mus musculus | 1 . . . 2914 | 2421/2923 (82%) | 0.0 |
| | (Mouse), 2910 aa. | 1 . . . 2910 | 2553/2923 (86%) | |
| CAA00831 | VON WILLEBRAND FACTOR - | 139 . . . 1233 | 392/1136 (34%) | 0.0 |
| | synthetic construct, 2324 aa | 35 . . . 1131 | 561/1136 (48%) | |
| | (fragment). | | | |
| P04275 | Von Willebrand factor precursor | 139 . . . 1233 | 394/1137 (34%) | 0.0 |
| | (vWF) - Homo sapiens (Human), | 35 . . . 1131 | 561/1137 (48%) | |
| | 2813 aa. | | | |
| Q28295 | Von Willebrand factor precursor | 139 . . . 1233 | 390/1138 (34%) | 0.0 |
| | (vWF) - Canis familiaris (Dog), | 35 . . . 1131 | 554/1138 (48%) | |
| | 2813 aa. | | | |
| A43932 | mucin 2 precursor, intestinal - | 137 . . . 1236 | 365/1126 (32%) | e−175 |
| | human, 3020 aa (fragment). | 35 . . . 1125 | 554/1126 (48%) | |

PFam analysis indicates that the NOV17a protein contains the domains shown in the Table 17E.

TABLE 17E
Domain Analysis of NOV17a

| | | Identities/ | | |
|---|---|---|---|---|
| | NOV17a | Similarities | | |
| | Match | for the Matched | | |
| Pfam Domain | Region | Region | | Expect Value |
| Arthro_defensin: | 87 . . . 116 | 10/36 | (28%) | 7.9 |
| domain 1 of 1 | | 17/36 | (47%) | |
| vwd: | 139 . . . 289 | 58/165 | (35%) | 1.9e−38 |
| domain 1 of 4 | | 117/165 | (71%) | |
| IBR: | 357 . . . 419 | 6/78 | (8%) | 7 |
| domain 1 of 1 | | 41/78 | (53%) | |
| EB: | 409 . . . 457 | 15/56 | (27%) | 9.7 |
| domain 1 of 1 | | 28/56 | (50%) | |
| TIL: | 409 . . . 463 | 19/70 | (27%) | 0.096 |
| domain 1 of 5 | | 37/70 | (53%) | |
| TILa: | 462 . . . 514 | 15/57 | (26%) | 2.8 |
| domain 1 of 1 | | 30/57 | (53%) | |
| vwc: | 465 . . . 525 | 18/91 | (20%) | 0.0087 |
| domain 1 of 1 | | 45/91 | (49%) | |
| vwd: | 503 . . . 658 | 62/168 | (37%) | 6.1e−45 |
| domain 2 of 4 | | 130/168 | (77%) | |
| TIL: | 769 . . . 833 | 19/76 | (25%) | 2.7e−06 |
| domain 2 of 5 | | 40/76 | (67%) | |
| TIL: | 873 . . . 935 | 18/74 | (24%) | 0.011 |
| domain 3 of 5 | | 42/74 | (57%) | |
| vwd: | 975 . . . 1121 | 45/166 | (27%) | 1.1e−32 |
| domain 3 of 4 | | 111/166 | (67%) | |
| TIL: | 1398 . . . 1453 | 16/69 | (23%) | 0.05 |
| domain 4 of 5 | | 40/69 | (58%) | |
| vwd: | 2101 . . . 2255 | 38/166 | (23%) | 3.9e−21 |
| domain 4 of 4 | | 111/166 | (67%) | |
| Chitin_bind_2: | 2333 . . . 2381 | 10/61 | (16%) | 7 |
| domain 1 of 1 | | 27/61 | (44%) | |
| TIL: | 2362 . . . 2423 | 20/71 | (28%) | 0.0095 |
| domain 5 of 5 | | 48/71 | (68%) | |
| Cys_knot: | 2822 . . . 2880 | 12/63 | (19%) | 0.06 |
| domain 1 of 1 | | 44/63 | (70%) | |

### Example 18

The NOV18 clone was analyzed, and the nucleotide and polypeptide sequences are shown in Table 18A.

TABLE 18A
NOV18 Sequence Analysis

| | SEQ ID NO:83 | 1882 bp |
|---|---|---|
| NOV18a, | CCCTCCCACGGCCCCTCCCGGCCTCTCTACATAAAGCCGGGGGTACTGGGCCTCAGGT | |
| C059293-01 DNA | CAGGCCTATGGCCATGGCCTTCACACACCTGCTCGATGCTCTGGGCAGCATGGGCCGC | |
| Sequence | TTCCAGCTCAACCACACAGCCCTGCTGCTGCTGCCCTGCGGCCTGCTGGCCTCCCACA | |
| | ACTTCCTGCAGAACTTCACCCCCGCTGTCCCCCCCCACCACTGCCGGGCCCCTGCCAA | |
| | CCACACTGAGGCCTCCACCAACGACTCCGGCGCCTGGCTGAGGGCCACCATACCCCTG | |
| | GACCAGCTTGGGGCCCCTGAGCCCTGCCGGCGCTTCACCAAGCCTCAGTGGGCCCTGC | |
| | TGAGCCCCAACTCCTCCATCCCGGGCGCGGCCACGGAGGGCTGCAAGGACGGCTGGGT | |
| | CTATAACCGCAGTGTTTTCCCGTCCACCATCGTGATGGAGTGGGATCTGGTGTGTGAG | |
| | GCCCGCACTCTCCGAGACCTGGCGCAGTCCGTCTACATTGCCGGGGTGCTGGTGGGGG | |
| | CTGCCGTGTTTGGCAGCTTGGCAGACAGGCTGGGCTGCAAGGGCCCCCTGGTCTGGTC | |
| | CTACCTGCAGCTGGCAGCTTCGGGGGCCGCCACAGCGTATTTCAGCTCCTTCAGTGCC | |
| | TATTGCGTCTTCCGGTTCCTGATGGGCATGACCTTCTCTGGCATCATCCTCAACTCCG | |
| | TCTCCCTGGTGATTGTGGAGTGGATGCCCACACGGGGCCGGACTGTGGCGGGTATTTT | |
| | GCTGGGGTATTCCTTCACCCTGGGCCAGCTCATCCTGGCTGGGGTAGCCTACCTGATT | |
| | CGCCCCTGGCGGTGCCTGCAGTTTGCCATCTCTGCTCCTTTCCTGATCTTTTTCCTCT | |
| | ATTCTTGGTGGCTTCCAGAGTCATCCCGCTGGCTCCTCCTGCATGGCAAGTCCCAGTT | |
| | AGCTGTACAGAATCTGCAGAAGGTGGCTGCAATGAACGGGAGGAAGCAGGAAGGGGAA | |
| | AGGCTGACCAAGGAGGTGATGAGCTCCTACATCCAAAGCGAGTTTGCAAGTGTCTGCA | |
| | CCTCCAACTCAATCTTGGACCTCTTCCGAACCCCGGCCATCCGCAAGGTCACATGCTG | |
| | TCTCATGGTGATTTGGTTCTCCAACTCTGTGGCTTACTATGGCCTGGCCATGGACCTG | |
| | CAGAAGTTTGGGCTCAGCCTATACCTGGTGCAGGCCCTGTTTGGAATCATCAACATCC | |
| | CGGCCATGCTGGTGGCCACCGCCACCATGATTTACGTGGGCCGCCGTGCCACGGTGGC | |
| | CTCCTTCCTCATCCTGGCCGGGCTCATGGTGATCCCCAACATGTTTGTGCCAGAAGGC | |
| | TCCACCCCCTCTCCACTACACCCTGGGCCTCCCTCTCCTCCCCTCCTCCTCAGCTGCA | |
| | CCCTACTCCCCTGTCTAGGCACGCAGATCCTGTGCACAGCCCAGGCAGCGCTGGGCAA | |
| | AGGCTGCCTGGCCAGCTCCTTCATCTGTGTGTACCTGTTTACCGGCGAGCTGTACCCC | |
| | ACGGAGATCAGGCAGATGGGGATGGGCTTTGCCTCTGTCCACGCCCGCCTCGGGGGCC | |
| | TGACGGCGCCCCTGGTTACCACACTTGGGGAATACAGCACCATCCTGCCACCCGTGAG | |
| | CTTTCGGGCCACCGCAATCCTGGCTGGGCTGGCCGTCTGCTTCCTGACTGAGACCCGC | |
| | AACATGCCCCTGGTCGAGACCATCGCAGCCATGGAGAGGAGGGTCAAAGAAGGCTCTT | |
| | CCAAGAAACATGTAGAAGAGAAGAGTGAAGAAATTTCTCTTCAGCAGCTGAGAGCATC | |
| | TCCCCTCAAAGAGACCATCTAAGCTGCCTGGAACCTGGTGCTTGCTAGCAGCACCTGA | |
| | GCCGATGTCCAGACGGCCCCCTGGCG | |
| | ORF Start: ATG at 66 | ORF Stop: TAA at 1818 |
| | SEQ ID NO:84 | 584 aa MW at 63391.5 kD |

| | |
|---|---|
| NOV18a, | MAMAFTDLLDALGSMGRFQLNHTALLLLPCGLLACHNFLQNFTAAVPPHHCRGPANHT |
| CG59293-01 Protein | EASTNDSGAWLRATIPLDQLGAPEPCRRFTKPQWALLSPNSSIPGAATEGCKDGWVYN |
| Sequence | RSVFPSTIVMEWDLVCEARTLRDLAQSVYIAGVLVGAAVFGSLADRLGCKGPLVWSYL |
| | QLAASGAATAYFSSFSAYCVFRFLMGMTFSGIILNSVSLVIVEWMPTRGRTVAGILLG |
| | YSFTLGQLILAGVAYLIRPWRCLQFAISAPFLIFFLYSWWLPESSRWLLLHGKSQLAV |
| | QNLQKVAAMNGRKQEGERLTKEVMSSYIQSEFASVCTSNSILDLFRTPAIRKVTCCLM |
| | VIWFSNSVAYYGLAMDLQKFGLSLYLVQALFGIINIPANLVATATMIYVGRRATVASF |
| | LILAGLMVIANMFVPEGSTPSPLHPGPPSPPLLLSCTLLPCLGTQILCTAQAALGKGC |
| | LASSFICVYLFTGELYPTEIRQMGMGFASVHARLGGLTAPLVTTLGEYSTILPPVSFG |
| | ATAILAGLAVCFLTETRNMPLVETIAAMERRVKEGSSKKHVEEKSEEISLQQLRASPL |
| | KETI |

Further analysis of the NOV18a protein yielded the following properties shown in Table 18B.

TABLE 18B
Protein Sequence Properties NOV18a

| | |
|---|---|
| PSort | 0.6000 probability located in plasma membrane; 0.4000 |
| analysis: | probability located in Golgi body; 0.3142 probability |
| | located in mitochondrial inner membrane; 0.3000 |
| | probability located in endoplasmic reticulum (membrane) |
| SignalP | Cleavage site between residues 35 and 36 |
| analysis: | |

A search of the NOV18a protein against the Geneseq database, a proprietary database that contains sequences published in patents and patent publication, yielded several homologous proteins shown in Table 18C.

TABLE 18C
Geneseq Results for NOV18a

| | | NOV18a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAW88488 | Rat organic anion transporter | 3 . . . 573 | 249/573 (43%) | e−137 |
| | OAT-1 - Rattus sp, 551 aa. | 1 . . . 542 | 346/573 (59%) | |
| | [WO9853064-A1, 26-NOV-1998] | | | |
| AAB47271 | hOAT1 - Homo sapiens, 550 aa. | 3 . . . 573 | 254/573 (44%) | e−137 |
| | [WO200104283-A2, 18-JAN-2001] | 1 . . . 541 | 345/573 (59%) | |
| AAY44278 | Human organic anion transporter - | 3 . . . 573 | 254/573 (44%) | e−137 |
| | Homo sapiens, 550 aa. [WO9964459- | 1 . . . 541 | 345/573 (59%) | |
| | A2, 16-DEC-1999] | | | |
| AAW88489 | Human organic anion transporter | 3 . . . 553 | 250/553 (45%) | e−137 |
| | OAT-1 - Homo sapiens, 563 aa. | 1 . . . 522 | 336/553 (60%) | |
| | [WO9853064-A1, 26-NOV-1998] | | | |
| AAY92903 | Rat cerebral organic anion transporter | 3 . . . 573 | 256/577 (44%) | e−135 |
| | OAT3 protein - Rattus sp, 536 aa. | 1 . . . 533 | 352/577 (60%) | |
| | [WO200017237-A1, 30-MAR-2000] | | | |

In a BLAST search of public sequence databases, the NOV18a protein was found to have homology to the proteins shown in the BLASTP data in Table 18D.

TABLE 18D
Public BLASTP Results for NOV18a

| | | NOV18a | Identities/ | |
|---|---|---|---|---|
| Protein | | Residues/ | Similarities for | |
| Accession | | Match | the Matched | Expect |
| Number | Protein/Organism/Length | Residues | Portion | Value |
| O57379 | RENAL ORGANIC ANION | 3 . . . 583 | 262/592 (44%) | e−139 |
| | TRANSPORTER - Pseudopleuronecta | 1 . . . 561 | 362/592 (60%) | |
| | americanus (Winter flounder), 562 aa. | | | |
| O35956 | RENAL ORGANIC ANION | 3 . . . 573 | 250/573 (43%) | e−137 |
| | TRANSPORT PROTEIN 1 - Rattus | 1 . . . 542 | 348/573 (60%) | |
| | norvegicus (Rat), 551 aa. | | | |
| Q9TSY7 | RENAL ORGANIC ANION | 3 . . . 573 | 253/573 (44%) | e−136 |
| | Oryctolagus cuniculus (Rabbit), 551 | 1 . . . 542 | 344/573 (59%) | |
| | aa. | | | |
| O95742 | RENAL ORGANIC ANION | 3 . . . 553 | 250/553 (45%) | e−136 |
| | TRANSPORT PROTEIN 1 - Homo | 1 . . . 522 | 336/553 (60%) | |
| | sapiens (Human), 563 aa. | | | |
| Q9R1U7 | ORGANIC ANION TRANSPORTER | 3 . . . 573 | 256/577 (44%) | e−134 |
| | 3 - Rattus norvegicus (Rat), 536 aa. | 1 . . . 533 | 352/577 (60%) | |

PFam analysis indicates that the NOV18a protein contains the domains shown in the Table 18E.

TABLE 18E
Domain Analysis of NOV18a

| | | Identities/ | | |
|---|---|---|---|---|
| | NOV18a | Similarities | | |
| | Match | for the Matched | | Expect |
| Pfam Domain | Region | Region | | Value |
| Chal_stil_syntC: domain | 200 . . . 212 | 5/13 | (38%) | 9.8 |
| 1 of 1 | | 11/13 | (85%) | |
| sugar_tr: domain 1 of 1 | 100 . . . 548 | 102/528 | (19%) | 5.9e−07 |
| | | 307/528 | (58%) | |

### Example 19

The NOV19 was analyzed, and the nucleotide and polypeptide sequences are shown in Table 19A.

TABLE 19A
NOV19 Sequence Analysis

| | SEQ ID NO:85 | 1802 bp |
|---|---|---|
| NOV19a, | TGGGGGAAACAGGCCCGTTGCCCTGGCCTCTTTCCCCTGGGCCAGCCTTTGTGAAGTG | |
| C059284-01 DNA | GGCCCCTCTTCTGGGCCCCTTGAGTAGGTTCCATGGCATTTTCTGAACTCCTGGACCT | |
| Sequence | CGTGGGTGGCCTGGGCAGGTTCCAGGTTCTCCAGACGATGGCTCTGATGGTCTCCATC | |
| | ATGTGGCTGTGTACCCAGAGCATGCTGGAGAACTTCTCGGCCGCCGTGCCCAGCCACC | |
| | GCTGCTGGGCACCCCTCCTGGACAACAGCACGGCTCAGGCCAGCATCCTAGGGAGCTT | |
| | GAGTCCTGAGGCCCTCCTGCCTATTTCCATCCCGCCGGGCCCCAACCAGAGGCCCCAC | |
| | CAGTGCCGCCGCTTCCGCCAGCCACAGTGGCAGCTCTTGGACCCCAATGCCACGGCCA | |
| | CCAGCTGGAGCGAGGCCGACACGGAGCCGTGTGTGGATGGCTGGGTCTATGACCGCAG | |
| | CATCTTCACCTCCACAATCGTGGCCAAGTGGAACCTCGTGTGTGACTCTCATGCTCTG | |
| | AAGCCCATGGCCCAGTCCATCTACCTGGCTGGCATTCTGGTGGGAGCTGCTGCGTGCG | |
| | GCCCTGCCTCAGACAGGTTTGGGCGCAGGCTGGTGCTAACCTGCAGCTACCTTCAGAT | |
| | GGCTGTGATGGGTACGGCAGCTGCCTTCGCCCCTGCCTTCCCCGTGTACTGCCTGTTC | |
| | CCCTTCCTGTTGGCCTTTGCCGTGGCAGGCGTCATGATGAACACGGGCACTCTCCTGA | |
| | TGGAGTGCACGCCGGCACGGGCCCGACCCTTGGTGATGACCTTOAACTCTCTGGGCTT | |
| | CAGCTTCGGCCATGGCCTGACAGCTGCAGTGGCCTACGGTGTGCGGGACTCCACACTG | |
| | CTGCAGCTGGTGGTCTCGGTCCCCTTCTTCCTCTGCTTTTTGTACTCCTCGTGGCTCG | |
| | CAGAGTCGGCACGATGGCTCCTCACCACAGGCAGGCTGGATTGGGGCCTGCAGGAGCT | |
| | GTGGAGGGTCGCTGCCATCAACGGAAAGGGGCCAGTGCAGGACACCCTGACCCCTGAG | |
| | GTCTTGCTTTCAGCCATGCGGGAGGAGCTGAGCATGGGCCAGCCTCCTGCCAGCCTGG | |
| | GCACCCTGCTCCGCATGCCCGGACTGCGCTTCCGGACCTGTATCTCCACGTTGTGCTG | |
| | GTTCGCCTTTGGCTTCACCTTCTTCGGCCTGGCCCTGGACCTGCAGGCCCTGGGCAGC | |
| | AACATCTTCCTGCTCCAAATGTTCATTGGTGTCGTGGACATCCCAGCCAAGATGGGCG | |
| | CCCTGCTGCTGCTGACCCACCTGGGCCGCCGCCCCACGCTGGCCGCATCCCTGTTGCT | |
| | GGCGGGGCTCTGCATTCTGGCCAACACGCTGGTGCCCCACGAAATGGGGGCTCTGCGC | |
| | TCAGCCTTGGCCGTGCTGGGGCTGGGCGGGGTGGGGGCTGCCTTCACCTGCATCACCA | |
| | TCTACAGCAGCGAGCTCTTCCCCACTGTGCTCAGGATGACGGCAGTGGGCTTGGGCCA | |
| | GATGGCAGCCCGTCGAGGAGCCATCCTGGGGCCTCTGGTCCGGCTGCTGGGTGTCCAT | |
| | GGCCCCTGGCTGCCCTTGCTGGTGTATGGGACGGTGCCAGTGCTGAGTGGCCTGGCCG | |
| | CACTGCTTCTGCCCGAGACCCAGAGCTTGCCGCTGCCCGACACCATCCAAGATGTGCA | |
| | GAACCAGGCAGTAAAGAAGGCAACACATGGCACGCTGGGGAACTCTGTCCTAAAATCC | |
| | ACACAGTTTTAGCCTCCTCGGGAACCTGCGATGGGACGGTCAGAGGAAGAGACTTCTT | |
| | CTGT | |
| | ORF Start: ATG at 91 | ORF Stop: TAG at 1750 |
| | SEQ ID NO:86 | 553 aa MW at 59629.4 kD |

| | |
|---|---|
| NOV19a, | MAFSELLDLVGGLGRFQVLQTMALMVSIMWLCTQSMLENFSAAVPSHRCWAPLLDNST |
| CG59284-01 Protein | AQASILGSLSPEALLAISIPPGPNQRPHQCRRFRQPQWQLLDPNATATSWSEADTEPC |
| Sequence | VDGWVYDRSIFTSTIVAKWNLVCDSHALKPMAQSIYLAGILVGAAACGPASDRFGRRL |
| | VLTWSYLQMAVMGTAAAFAPAFPVYCLFRFLLAFAVAGVMMNTGTLLMEWTAARARPL |
| | VNTLNSLGFSFGHGLTAAVAYGVRDWTLLQLVVSVPFFLCFLYSWWLAESARWLLTTG |
| | RLDWGLQELWRVAAINGKGAVQDTLTPEVLLSAMREELSMGQPPASLGTLLRMPGLRF |
| | RTCISTLCWFAFGFTFFGLALDLQALGSNIFLLQMFIGVVDIPAKMGALLLLSHLGRR |
| | PTLAASLLLAGLCILANTLVPHEMGALRSALAVLGLGGVGAAFTCITIYSSELFPTVL |
| | RMTAVGLGQMAARGGAILGPLVRLLGVHGPWLPLLVYGTVPVLSGLAALLLPETQSLP |
| | LPDTIQDVQNQAVKKATHGTLGNSVLKSTQF |

Further analysis of the NOV19a protein yielded the following properties shown in Table 19B.

TABLE 19B
Protein Sequence Properties NOV19a

| | |
|---|---|
| PSort | 0.6000 probability located in plasma membrane; 0.4000 |
| analysis: | probability located in Golgi body; 0.3000 probability |
| | located in endoplasmic reticulum (membrane); |
| | 0.3000 probability located in microbody (peroxisome) |
| SignalP | Cleavage site between residues 44 and 45 |
| analysis: | |

A search of the NOV19a protein against the Geneseq database, a proprietary database that contains sequences published in patents and patent publication, yielded several homologous proteins shown in Table 19C.

TABLE 19C
Geneseq Results for NOV19a

| | | NOV19a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE10332 | (TRICH-9) protein - Homo sapiens, | 1 . . . 553 | 553/553 (100%) | 0.0 |
| | 553 aa. [WO200162923-A2, | | | |
| | 30-AUG-2001] | | | |
| AAE06571 | Human protein having hydrophobic | 1 . . . 553 | 552/578 (95%) | 0.0 |
| | domain, HP03613 - Homo sapiens, | 1 . . . 578 | 553/578 (95%) | |
| | 578 aa. [WO200149728-A2, | | | |
| | 12-JUL-2001] | | | |
| AAE06612 | Human protein having hydrophobic | 1 . . . 533 | 284/534 (53%) | e−161 |
| | domain, HP03882 - Homo sapiens, | 1 . . . 530 | 372/534 (69%) | |
| | 550 aa. [WO200149728-A2, | | | |
| | 12-JUL-2001] | | | |
| AAB69091 | Human organic anion transporter OAT4 | 1 . . . 533 | 284/534 (53%) | e−161 |
| | protein sequence SEQ ID NO:2 - Homo | 1 . . . 530 | 372/534 (69%) | |
| | sapiens, 550 aa. [WO200102562-A1, | | | |
| | 11-JAN-2001] | | | |
| AAE10336 | Human transporter and ion channel-13 | 1 . . . 533 | 282/549 (51%) | e−157 |
| | (TRICH-13) protein - Homo sapiens, | 1 . . . 546 | 370/549 (67%) | |
| | 566 aa. [WO200162923-A2, | | | |
| | 30-AUG-2001] | | | |

In a BLAST search of public sequence databases, the NOV19a protein was found to have homology to the proteins shown in the BLASTP data in Table 19D.

TABLE 19D
Public BLASTP Results for NOV19a

| | | NOV19a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96S37 | RST - Homo sapiens (Human), | 1 . . . 553 | 553/553 (100%) | 0.0 |
| | 553 aa. | 1 . . . 553 | 553/553 (100%) | |
| CAC51145 | SEQUENCE 21 FROM PATENT | 1 . . . 553 | 552/578 (95%) | 0.0 |
| | WO0149728 - Homo sapiens | 1 . . . 578 | 553/578 (95%) | |
| | (Human), 578 aa. | | | |
| Q96DT2 | ORGANIC ANION TRANSPORTER | 1 . . . 553 | 507/561 (90%) | 0.0 |
| | 4 LIKE PROTEIN - Homo sapiens | 1 . . . 552 | 513/561 (91%) | |
| | (Human), 552 aa. | | | |
| O54778 | RST - Mus musculus (Mouse), | 1 . . . 552 | 409/552 (74%) | 0.0 |
| | 553 aa. | 1 . . . 552 | 462/552 (83%) | |
| Q9NSA0 | ORGANIC ANION | 1 . . . 533 | 284/534 (53%) | e−160 |
| | TRANSPORTER 4 (OAT4) - Homo | 1 . . . 530 | 372/534 (69%) | |
| | sapiens (Human), 550 aa. | | | |

PFam analysis indicates that the NOV19a protein contains the domains shown in the Table 19E.

TABLE 19E
Domain Analysis of NOV19a

| | NOV19a | Identities/ | | |
|---|---|---|---|---|
| | Match | Similarities for | | Expect |
| Pfam Domain | Region | the Matched Region | | Value |
| zf-RanBP: domain 1 of 1 | 26 . . . 55 | 7/32 | (22%) | 5.8 |
| | | 17/32 | (53%) | |
| sugar_tr: domain 1 of 1 | 106 . . . 530 | 89/493 | (18%) | 4.2e−07 |
| | | 277/493 | (56%) | |

### Example 20

The NOV20 clone was analyzed, and the nucleotide and polypeptide sequences are shown in Table 20A.

TABLE 20A
NOV20 Sequence Analysis

| | SEQ ID NO:87 | 1050 bp |
|---|---|---|
| NOV20a, | CTTGAAGTAATTTATATTCTATCTTTATCGCTTGTTCCTAGGAATCATGGATCACGTC | |
| CG59278-01 DNA | AGTCATAACTGGACTCAGAGTTTTATCCTTGCTGGTTTCACCACCACTGGGACCCTAC | |
| Sequence | AACCTCTTGCCTTCTTGGGGACCCTATGCATCTATCTCCTCACACTTGCAGGGAACAT | |
| | TCTCATCATTGTCCTGGTACAGTTAGATTCTGGACTGTTCACGCCCATGTACTTATTT | |
| | ATCAGTGTCCTCTCCTTTGTAGAGGTGTGGTATGTCAGCACCACAGTGCCCATGCTGC | |
| | TGCACACCTTGCTCCAAGGGTGTTCACCCGTCTCATCAGCTGTATGCTTTATTCAGCT | |
| | ATCCTTTCATTCCTTAGGGATGACTGAGTGCTACCTGCTGGGTGTCATGGCACTGGAT | |
| | AGCTACCTTATCATCTGCCACCCACTCCACTACCACGCACTCATGAGCAGACAGGTAC | |
| | AGTTACGACTAGCTGGGGCCAGTTGGGTGGCTGGCTTCTCAGCTGCACTTGTGCCAGC | |
| | CACCCTCACTGCCACTCTGCCCTTCTGCTTGAAAGAGGTGGCCCATTACTTTTGTGAC | |
| | TTGGCACCACTAATGCGGTTGGCATGTGTGGACACAAGCTGGCATGCTAGGGCCCATG | |
| | GCACAGTGATTGGTGTGGCCACTGGTTGCAACTTTGTGCTCATTTTGGGACTCTATGG | |
| | AGGTATCCTGAATGCTGTGCTGAAGCTACCCTCAGCTGCCAGTAGTGCCAAGGCCTTC | |
| | TCTACCTGTTCCTCCCACGTAACTGTGGTGGCACTATTCTATGCTTCTGCCTTCACAG | |
| | TATATGTGGGCTCACCTGGGAGTCGACCTGAGAGCACAGACAAGCTTGTTGCCTTGGT | |
| | TTATGCCCTTATTACCCCTTTCCTCAATCCTATCATCTATAGCCTTCGCAACAAGGAG | |
| | GTGAAGAGGCTTTAAGGAGAGTCATGGCTGGGCGCGGTGGCTCACGCCTGTAACCC | |
| | AGCACTCTGGGAGGCCGAGGCGGGTGGATCACGAGGTCAGGAGATCGAGACCACGGTG | |
| | AAACCC | |
| | ORE Start: ATG at 47 | ORF Stop: TAA at 980 |
| | SEQ ID NO:88 | 311 aa MW at 33602.3 kD |

| | |
|---|---|
| NOV20a, | MDHVSHNWTQSFILAGFTTTGTLQPLAFLGTLCIYLLTLAGNILIIVLVQLDSGLFTP |
| CG59278-01 Protein | MYLFISVLSFVEVWYVSTTVPMLLHTLLQGCSPVSSAVCFIQLCFHSLGMTECYLLGV |
| Sequence | MALDSYLIICHPLHYHALMSRQVQLRLAGASWVAGFSAALVPATLTATLPFCLKEVAH |
| | YFCDLAPLMRLACVDTSWHARAHGTVIGVATGCNFVLILGLYGGILNAVLKLPSAASS |
| | AKAFSTCSSHVTVVALFYASAFTVYVGSPGSRPESTDKLVALVYALITPFLNPIIYSL |
| | RNKEVKKALRRVMAGRGGSRL |

Further analysis of the NOV20a protein yielded the following properties shown in Table 20B.

TABLE 20B
Protein Sequence Properties NOV20a

| | |
|---|---|
| PSort | 0.6400 probability located in plasma membrane; 0.5000 |
| analysis: | probability located in microbody (peroxisome); 0.4600 |
| | probability located in Golgi body; 0.3700 |
| | probability located in endoplasmic reticulum (membrane) |
| SignalP | Cleavage site between residues 41 and 42 |
| analysis: | |

A search of the NOV20a protein against the Geneseq database, a proprietary database that contains sequences published in patents and patent publication, yielded several homologous proteins shown in Table 20C.

TABLE 20C
Geneseq Results for NOV20a

| | | NOV20a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAG71821 | Human olfactory receptor polypeptide, | 1 . . . 302 | 300/303 (99%) | e−172 |
| | SEQ ID NO: 1502 - Homo sapiens, | 1 . . . 303 | 301/303 (99%) | |
| | 303 aa. [WO200127158-A2, | | | |
| | 19-APR-2001] | | | |
| AAG72628 | Murine OR-like polypeptide query | 1 . . . 302 | 254/303 (83%) | e−148 |
| | sequence, SEQ ID NO: 2309 - Mus | 6 . . . 308 | 277/303 (90%) | |
| | musculus, 333 aa. [WO200127158-A2, | | | |
| | 19-APR-2001] | | | |
| AAG72627 | Murine OR-like polypeptide query | 1 . . . 266 | 204/267 (76%) | e−117 |
| | sequence, SEQ ID NO: 2308 - Mus | 14 . . . 280 | 227/267 (84%) | |
| | musculus, 282 aa. [WO200127158-A2, | | | |
| | 19-APR-2001] | | | |
| AAG71814 | Human olfactory receptor polypeptide, | 1 . . . 307 | 131/309 (42%) | 6e−69 |
| | SEQ ID NO: 1495 - Homo sapiens, | 1 . . . 309 | 188/309 (60%) | |
| | 317 aa. [WO200127158-A2, | | | |
| | 19-APR-2001] | | | |
| AAG72355 | Human OR-like polypeptide query | 12 . . . 303 | 138/294 (46%) | 5e−68 |
| | sequence, SEQ ID NO: 2036 - Homo | 11 . . . 304 | 181/294 (60%) | |
| | sapiens, 312 aa. [WO200127158-A2, | | | |
| | 19-APR-2001] | | | |

In a BLAST search of public sequence databases, the NOV20a protein was found to have homology to the proteins shown in the BLASTP data in Table 20D.

TABLE 20D
Public BLASTP Results for NOV20a

| | | Residues/ | Similarities for | |
|---|---|---|---|---|
| Accession | | Match | the Matched | |
| Number | Protein/Organism/Length | Residues | Portion | Value |
| Q96KK4 | DJ994E9.5 (OLFACTORY | 12 . . . 303 | 138/294 (46%) | 2e−67 |
| | RECEPTOR, FAMILY 10, | 5 . . . 298 | 181/294 (60%) | |
| | SUBFAMILY C, MEMBER 1 | | | |
| | (HS6M1-17)) - Homo sapiens (Human), | | | |
| | 306 aa. | | | |
| Q96KK4 | Olfactory receptor 10C1 | 12 . . . 303 | 138/294 (46%) | 2e−67 |
| | (Hs6M1-17) - Homo sapiens (Human), | 11 . . . 304 | 181/294 (60%) | |
| | 312 aa. | | | |
| Q63394 | OL1 RECEPTOR - Rattus norvegicus | 1 . . . 303 | 129/305 (42%) | 1e−62 |
| | (Rat), 313 aa. | 1 . . . 305 | 192/305 (62%) | |
| Q9JKA6 | OLFACTORY RECEPTOR P2 - Mus | 7 . . . 303 | 135/302 (44%) | 2e−61 |
| | musculus (Mouse), 315 aa. | 5 . . . 306 | 178/302 (58%) | |
| Q9H207 | Olfactory receptor 10A5 (HP3) | 7 . . . 301 | 135/302 (44%) | 3e−61 |
| | (Putative taste receptor JCG6) - | 5 . . . 304 | 181/302 (59%) | |
| | Homo sapiens (Human), 317 aa. | | | |

PFam analysis indicates that the NOV20a protein contains the domains shown in the Table 20E.

TABLE 20E
Domain Analysis of NOV20a

| | NOV20a | Identities/ | |
|---|---|---|---|
| | Match | Similarities for | Expect |
| Pfam domain | Region | the Matched Region | Value |
| 7tm_1: domain 1 of 1 | 41 . . . 288 | 54/268 (20%) | 2.7e−18 |
| | | 170/268 (63%) | |

### Example 21

The NOV21 clone was analyzed, and the nucleotide and polypeptide sequences are shown in Table 21A.

TABLE 21A
NOV21 Sequence Analysis

| | |
|---|---|
| SEQ ID NO:89 | 793 bp |

| | |
|---|---|
| NOV21a, | ACTTACTGCATTTGGTGCTGTGATCCAACTCATCTCCCCATCCCTGCAGAGAAACCTG |
| CG59274-01 DNA | TGACCATGAGGAGCAGCCTGACCATGGTGGGAACCCTCTCAGGCCTTCCTGTCCCTTGT |
| Sequence | TACTGCTGTGACCAGTTCTACCAGTTACTTCCTACCTTACTGGCTCTTTGGATCCCAG |
| | ATGGGGAAGCCAGTGTCATTCAGCACATTCCGGAGGTGCAACTACCCTGTGCGGGGAG |
| | AGGGACACAGTCTGATCATGGTGGAAGAATGTGGGCGCTATGCCAGCTTCAATGCCAT |
| | CCCAAGCCTGGCCTGGCAGATGTGCACAGTGGTGACAGGTGCCGGCTGTGCTCTCCTG |
| | CTCCTGGTGGCACTAGCTGCTGTCCTGGGTTGCTGCATGGAGGAGCTCATCTCCAGAA |
| | TGATGGGACGTTGCATGGGAGCAGCGCAGTTTGTTGGAGGGCTGCTGATAAGCTCAGG |
| | CTGTGCCTTATACCCTTTAGGATGGAATAGCCCGGAGATAATGCAAACATGTGGGAAT |
| | GTCTCCAATCAATTTCAGTTAGGTACCTGTCGGCTTGGCTGGGCCTATTACTGTGCTG |
| | GAGGTGGAGCAGCTGCAGCCATGTTGATCTGCACCTGGCTCTCTTCCTTTGCTGGAAG |
| | AAACCCCAAGCCTGTCATATTGGTGGAGAGCATCATGAGGAATACCAATTCTTATGCT |
| | ATGGAGCTTGACCATTGCCTCAAACCTTGAGCTTTGAAAGAAGATTGGAGAGGGTGGG |
| | AAAGGGGAGGAGGGAGCCCTGAAAAGAGGTACTAAGGAT |

| | ORF Start: ATG at 64 | ORF Stop: TGA at 724 |
|---|---|---|
| | SEQ ID NO:90 | 220 aa MW at 23776.7 kD |
| NOV21a, | MRSSLTMVGTLWAFLSLVTAVTSSTSYFLPYWLFGSQMGKPVSFSTFRRCNYPVRGEG | |
| C059274-01 Protein | HSLIMVEECGRYASFNAIPSLAWQMCTVVTGAGCALLLLVALAAVLGCCMEELISRMM | |
| Sequence | GRCMGAAQFVGGLLISSGCALYPLGWNSPEIMQTCGNVSNQFQLGTCRLGWAYYCAGG | |
| | GAAAAMLICTWLSCFAGRNPKPVILVESIMRNTNSYAMELDHCLKP | |
| | SEQ ID NO:91 | 793 bp |
| NOV21b, | ACTTACTGCATTTGGTGCTGTGATCCAACTCATCTCCCCATCCCTGCAGAGAAACCTG | |
| CG59274-02 DNA | TGACCATGAGGACCAGCCTGACCATGGTGGGAACCCTCTGGGCCTTCCTGTCCCTTGT | |
| Sequence | TACTGCTGTGACCAGTTCTACCAGTTACTTCCTACCTTACTGGCTCTTTGGATCCCAG | |
| | ATGGGGAAGCCAGTGTCATTCAGCACATTCCGGAGGTGCAACTACCCTGTGCGGGGAG | |
| | AGGGACACAGTCTGATCATGGTGGAAGAATGTGGGCGCTATGCCAGCTTCAATGCCAT | |
| | CCCAAGCCTGGCCTGGCAGATGTGCACAGTGGTGACAGGTGCCGGCTGTGCTCTGCTG | |
| | CTCCTGGTGGCACTAGCTGCTGTCCTGGGTTGCTGCATGGAGGAGCTCATCTCCAGAA | |
| | TGATGGGACGTTGCATGGGAGCAGCGCAGTTCGTTGGAGGGCTGCTGATAAGCTCAGG | |
| | CTGTGCCTTATACCCTTTAGCATGGAATAGCCCGGAGATAATGCAAACATGTGGGAAT | |
| | GTCTCCAATCAATTTCAGTTAGGTACCTGTCGGCTTGGCTGGGCCTATTACTGTCCTG | |
| | GAGGTGGAGCAGCTCCAGCCATGTTGATCTGCACCTCCCTCTCTTGCTTTGCTGGAAG | |
| | AAACCCCAAGCCTGTCATATTGGTGGAGAGCATCATGAGGAATACCAATTCTTATGCT | |
| | ATGGAGCTTGACCATTGCCTCAAACCTTGAGCTTTGAAAGAAGATTGGAGAGGGTGGG | |
| | AAAGGGGAGGAGGGAGCCCTGAAAAGAGGTACTAGGAT | |
| | ORF Start: ATG at 64 | ORF Stop: TGA at 724 |
| | SEQ ID NO:92 | 220 aa MW at 23776.7 kD |

| | |
|---|---|
| NOV21b, | MRSSLTMVGTLWAFLSLVTAVTSSTSYFLPYWLFGSQMGKPVSFSTFRRCNYPVRGEG |
| CG59274-02 Protein | HSLIMVEECCRYASFNAIPSLAWQMCTVVTGAGCALLLLVALAAVLGCCMEELISRMM |
| Sequence | GRCMGAAQFVGGLLISSGCALYPLGWNSPEIMQTCGNVSNQFQLGTCRLGWAYYCAGG |
| | GAAAAMLICTWLSCFAGRNPKPVILVESIMRNTNSYAMELDHCLKP |

Sequence comparison of the above protein sequences yields the following sequence relationships shown in Table 21B.

TABLE 21B
Comparison of NOV21a against NOV21b and NOV21c.

| | NOV21a Residues/Match | Identities/Similarities |
|---|---|---|
| Protein Sequence | Residues | for the Matched Region |
| NOV21b | 1 . . . 220 | 181/220 (82%) |
| | 1 . . . 220 | 181/220 (82%) |

Further analysis of the NOV21 a protein yielded the following properties shown in Table 21C.

TABLE 21C
Protein Sequence Properties NOV21a

| | |
|---|---|
| PSort | 0.4600 probability located in plasma membrane; 0.1000 |
| analysis: | probability located in endoplasmic |
| | reticulum (membrane); 0.1000 probability |
| | located in endoplasmic reticulum (lumen); |
| | 0.1000 probability located in outside |
| SignalP | Likely cleavage site between residues 21 and 22 |
| analysis: | |

A search of the NOV21 a protein against the Geneseq database, a proprietary database that contains sequences published in patents and patent publication, yielded several homologous proteins shown in Table 21D.

TABLE 21D
Geneseq Results for NOV21a

| | | NOV21a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU14297 | Human novel protein #168 - Homo | 1 . . . 220 | 216/261 (82%) | e−123 |
| | sapiens, 261 aa. [WO200155437-A2, | 1 . . . 261 | 219/261 (83%) | |
| | 02-AUG-2001] | | | |
| AAU14533 | Human novel protein #404 - Homo | 1 . . . 199 | 199/199 (100%) | e−117 |
| | sapiens, 239 aa. [WO200155437-A2, | 1 . . . 199 | 199/199 (100%) | |
| | 02-AUG-2001] | | | |
| AAU14532 | Human novel protein #403 - Homo | 1 . . . 199 | 199/199 (100%) | e−117 |
| | sapiens, 239 aa. [WO200155437-A2, | 1 . . . 199 | 199/199 (100%) | |
| | 02-AUG-2001] | | | |
| AAU14296 | Human novel protein #167 - Homo | 1 . . . 199 | 195/199 (97%) | e−115 |
| | sapiens, 269 aa. [WO200155437-A2, | 1 . . . 199 | 196/199 (97%) | |
| | 02-AUG-2001] | | | |
| AAB80378 | Secreted protein encoded by gene #8 - | 1 . . . 195 | 125/196 (63%) | 5e−77 |
| | Homo sapiens, 200 aa. | 1 . . . 196 | 151/196 (76%) | |
| | [WO200107459-A1, 01-FEB-2001] | | | |

In a BLAST search of public sequence databases, the NOV21a protein was found to have homology to the proteins shown in the BLASTP data in Table 21E.

TABLE 21E
Public BLASTP Results for NOV21a

| | | NOV21a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9Y693 | PARTNER - Homo sapiens (Human), | 1 . . . 196 | 151/196 (76%) | 2e−76 |
| | 200 aa. | | | |
| Q96SH5 | BA183L8.1 (LIPOMA HMGIC | 1 . . . 127 | 79/128 (61%) | 3e−43 |
| | FUSION PARTNER) - Homo sapiens | 1 . . . 128 | 97/128 (75%) | |
| | (Human), 128 aa (fragment). | | | |
| Q92605 | KIAA0206 PROTEIN - Homo sapiens | 67 . . . 190 | 43/125 (34%) | 3e−15 |
| | (Human), 193 aa (fragment). | 47 . . . 170 | 63/125 (50%) | |
| Q9W068 | CG12026 PROTEIN (LP10272P) - | 8 . . . 197 | 49/195 (25%) | 2e−12 |
| | Drosophila melanogaster (Fruit fly), | 25 . . . 208 | 85/195 (43%) | |
| | 265 aa. | | | |
| Q955W9 | SD01285P - Drosophila melanogaster | 5 . . . 171 | 41/175 (23%) | 1e−11 |
| | (Fruit fly), 219 aa. | 6 . . . 178 | 77/175 (43%) | |

PFam analysis indicates that the NOV21 a protein contains the domains shown in the Table 21F.

TABLE 21F
Domain Analysis of NOV21a

| | | | |
|---|---|---|---|
| | | Identities/ | |
| | | Similarities for | Expect |
| Pfam Domain | NOV21a Match Region | the Matched Region | Value |

No Significant Matches Found

### Example 22

The NOV22 clone was analyzed, and the nucleotide and polypeptide sequences are shown in Table 22A.

TABLE 22A
NOV22 Sequence Analysis

| | SEQ ID NO:93 | 1228 bp |
|---|---|---|
| NOV22a, | AGATCTGTGACCAGTTCTACCAGTTACTTCCTACCTTACTGGCTCTTTGGATCCCAGA | |
| 172885510 DNA | TGGGGAAGCCAGTGTCATTCAGCACATTCCGGAGGTGCAACTACCCTGTGCGGGGAGA | |
| Sequence | GGGACACAGTCTGATCATGGTGGAAGAATGTGGGCGCTATGCCAGCTTCAATGCCATC | |
| | CCAAGCCTGGCCTGGCAGATGTGCACAGTGGTGACAGGTGCCGGCTGTCTCGAG | |
| | ORF Start: ATC at 3 | ORF Stop: G r at 228 |
| | SEQ ID NO:94 | 75 aa MW at 8305.5 kD |

| | |
|---|---|
| NOV22a, | ICDQFYQLLPTLLALWIPDGEASVIQHIPEVQLPCAGRGTQSDHGCRMWALCQLQCHP |
| 172885510 Protein | KPGLADVHSGDRCRLSR |
| Sequence | |

Further analysis of the NOV22a protein yielded the following properties shown in Table 22B.

TABLE 22B
Protein Sequence Properties NOV22a

| | |
|---|---|
| PSort | 0.3000 probability located in microbody (peroxisome); |
| analysis: | 0.3000 probability located in nucleus; 0.1000 |
| | probability located in mitochondrial matrix space; 0.1000 |
| | probability located in lysosome (lumen) |
| SignalP | No Known Signal Sequence Indicated |
| analysis: | |

A search of the NOV22a protein against the Geneseq database, a proprietary database that contains sequences published in patents and patent publication, yielded several homologous proteins shown in Table 22C.

TABLE 22C
Geneseq Results for NOV22a

| | | | | |
|---|---|---|---|---|
| | | NOV22a | Identities/ | |
| | Protein/Organism/ | Residues/ | Similarities | |
| Geneseq | Length | Match | for the | Expect |
| Identifier | [Patent #, Date] | Residues | Matched Region | Value |

No Significant Matches Found

In a BLAST search of public sequence databases, the NOV22a protein was found to have homology to the proteins shown in the BLASTP data in Table 22D.

TABLE 22D
Public BLASTP Results for NOV22a

| | | | | |
|---|---|---|---|---|
| | | NOV22a | Identities/ | |
| Protein | | Residues/ | Similarities | |
| Accession | Protein/Organism/ | Match | for the | Expect |
| Number | Length | Residues | Matched Portion | Value |

No Significant Matches Found

PFam analysis indicates that the NOV22a protein contains the domains shown in the Table 22E.

TABLE 22E
Domain Analysis of NOV22a

| | | Identities/ | | |
|---|---|---|---|---|
| | NOV22a | Similarities | | |
| | Match | for the Matched | | Expect |
| Pfam Domain | Region | Region | | Value |
| Man-6-P_recep: | 156 . . . 168 | 9/13 | (69%) | 0.7 |
| domain 1 of 1 | | 9/13 | (69%) | |
| perilipin: domain 1 of 1 | 10 . . . 369 | 139/411 | (34%) | 1.4e−76 |
| | | 240/411 | (58%) | |

### Example 23

The NOV23 clone was analyzed, and the nucleotide and polypeptide sequences are shown in Table 23A.

TABLE 23A
NOV23 Sequence Analysis

| | SEQ ID NO:95 | 1916 bp |
|---|---|---|
| NOV23a, | GCCGCTGCCGTCGCCCCTAGCCCCAGCAGCCCTGGTCTCGCAGCCTCCTGCGGCTCTG | |
| CG57734-01 DNA | GTCGCCCGACCAGCCATGTCTCTCGGCGGAGCCTCCGAGCGCAGCGTCCCGGCCACCA | |
| Sequence | AGATTGAAATTACCGTGTCCTGCCGGACCTTGATACCTTCTCCAAGTCCGACCCCAGT | |
| | AGGCGGCTCCAGGACCGGGAGGGCGAACTTGGGGTCTGGGCGCGACTCAGGGGCGGGT | |
| | GGAGTCGGGGCCAGGGGTGGGAGCCGACCTGACGTCCTTCCCTCCCCGCCCCCACCTG | |
| | CAGTGGTGGTGCTTTACACGCAGAGCCGGGCCAGCCAGGAGTGGCGGGAGTTCGGACG | |
| | GACCGAGGTGATTGATAACACGCTGAACCCAGACTTCGTGCGCAAATTCGTCCTCGAC | |
| | TATTTCTTTGAGGAAAAGCAAAATCTGCGCTTCGATGTGTACAACGTGGACTCCAAAA | |
| | CCAACATCTCCAAACCGAAGGATTTCCTGGGACAAGCGTTCCTGGCCCTGGGAGAGGT | |
| | GATTGGAGGCCAGGGCAGCCGAGTAGAGCGAACCCTCACGGGTGTACCAGGCAAGAAG | |
| | TGTGGGACCATATTGCTGACTGCAGAAGAGCTTAGCAATTGTCGGGACATTGCCACCA | |
| | TGCAGCTGTGTGCAAACAAGCTGGACAAGAAGGACTTCTTTGGGAAATCAGACCCCTT | |
| | CCTTGTGTTCTACAGGAGCAATGAGGATGGCACGTTCACCATCTGCCACAAGACAGAG | |
| | GTTGTGAAAAACACGCTGAATCCTGTGTGGCAGCCCTTCAGCATCCCTGTGCGGGCTC | |
| | TGTGCAATGGACACTATGACAGAACGGTGAAGATTGATGTGTACGACTGGGACCGGGA | |
| | TGGAAGCCACGATTTCATTGGTGAGTTCACCACCAGCTACCGGGAGCTGAGCAAGGCC | |
| | CAGAACCAGTTCACAGTATATGAGGTGCTTAACCCTCGGAAGAAATGTAAGAAGAAGA | |
| | AATATGTCAACTCAGGAACTGTGACGCTGCTCTCCTTCTCTGTGGACTCTGAATTCAC | |
| | TTTTGTTGATTACATCAAGGGAGGGACACAGCTGAACTTCACAGTAGCCATTGACTTC | |
| | ACGGCTTCCAATGGTAATCCTCTGCAGCCTACCTCCCTGCACTACATGAGTCCCTACC | |
| | AGCTCAGCGCCTATGCCATGGCCCTCAAGGCAGTGGGAGAGATCATCCAGGACTATGA | |
| | CAGTGATAAGCTCTTCCCAGCTTATGGCTTTGGGGCCAAGCTGCCCCCAGAGGGACGG | |
| | ATCTCCCACCAGTTCCCCCTGAACAACAATGATGAGGACCCCAACTGTGCGCGCATCG | |
| | AGGGTGTGCTGGAGAGCTATTTCCAGAGCCTGCGCACAGTGCAGCTCTATGGGCCCAC | |
| | CTACTTTGCTCCTGTCATCAACCAAGTGGCCAGGGCTGCAGCCAAGATCTCTGATGGC | |
| | TCCCAGTACTATGTTCTGCTCATCATCACTGATGGGGTCATCTCTGACATGACGCAGA | |
| | CCAAGGAGGCCATCGTCAGCGCCTCCTCATTGCCCATGTCTATCATTATCGTCGGTGT | |
| | AGGACCAGCCATGTTTGAGGCAATGGAAGAGTTGGACGGTGATGATGTGCGCGTGTCC | |
| | TCTAGGGGACGCTACGCAGAGCGGGACATCGTTCAGTTCGTCCCATTCCGAGACTATG | |
| | TTGACCGGTCGGGGAACCAGGTGTTGAGCATGGCCCGACTGGCCAAGGATGTGCTGGC | |
| | CGAGATCCCGGACCAGCTGCTGTCCTATATGCGCACCAGAGACATCCAGCCTCCGCCC | |
| | CCACCCCCTGCCAACCCCAGCCCGATCCCAGCTCCAGAGCAGCCCTGAGCATTCCACA | |
| | TATCCAATGCCTCACAGTCTGCAAGCCTGCTCACCCACTGCTTCTGCTTTAAGCCAGA | |
| | GG | |
| | ORF Start: ATG at 74 | ORE Stop: TGA at 1844 |
| | SEQ ID NO:96 | 590 aa MW at 65041.9 kD |
| NOV23a, | MSLGGASERSVPATKIEITVSCRTLIPSPSPTPVGGSRTGRGNLGSGRDSGAGGVGAR | |
| CG57734-01 Protein | GGSRPDVLPSPPPPAVVVLYTQSRASQEWREFGRTEVIDNTLNPDFVRKFVLDYFFEE | |
| Sequence | KQNLRFDVYNVDSKTNISKPKDFLGQAFLALGEVIGGQGSRVERTLTGVPGKKCGTIL | |
| | LTAEELSNCRDIATMQLCANKLDKKDFFGKSDPFLVFYRSNEDGTFTICHKTEVVKNT | |
| | LNPVWQPFSIPVRALCNGDYDRTVKIDVYDWDRDGSHDFIGEFTTSYRELSKAQNQFT | |
| | VYEVLNPRKKCKKKKYVNSGTVTLLSFSVDSEFTFVDYIKGGTQLNFTVAIDFTASNG | |
| | NPLQPTSLHYMSPYQLSAYAMALKAVGEIIQDYDSDKLFPAYGFGAKLPPEGRISHQF | |
| | PLHNMDEDPNCAGIEGVLESYFQSLRTVQLYGPTYFAPVINQVARAAAKTSDGSQYYV | |
| | LLIITDGVTSDMTQTKEAIVSASSLPMSIIIVGVGPAMFEAMEELDGDDVRVSSRGRY | |
| | AERDIVQFVPFRDYVDRSGNQVLSMARLAKDVLAEIPEQLLSYMRTRDIQPRPPPPAN | |
| | PSPIPAPEQP | |
| | SEQ ID NO:97 | 11742 bp |
| NOV23b, | CCGACCAGCCATGTCTCTCGGCGGAGCCTCCGAGCGCAGCGTCCCGCCCACCAAGATT | |
| CG5 7734-02 DNA | GAAATTACCGTGTCCTGCCGGAACCTGCTAGACCTCGATACCTTCTCCAAGTCCGACC | |
| Sequence | CCATGGTGGTGCTTTACACGCAGAGCCGGGCCAGCCAGGAGTGGCGGGAGTTCGGACG | |
| | GACCGACGTGATTGATAACACGCTGAACCCAGACTTCGTGCGCAAATTCGTCCTCCAC | |
| | TATTTCTTTGAGGAAAAGCAAAATCTGCGCTTCGATGTGTACAACGTGGACTCCAAAA | |
| | CCAACATCTCCAAACCGAAGGATTTCCTGGGACAAGCGTTCCTGGCCCTGGGAGAGGT | |
| | GATTGGAGGCCAGGGCAGCCGAGTAGAGCGAACCCTCACGGGTGTACCAGGCAAGAAG | |
| | TGTCGCACCATATTGCTGACTGCAGAAGAGCTTAGCAATTGTCGGGACATTGCCACCA | |
| | TGCAGCTGTGTGCAAACAAGCTGGACAAGAAGGACTTCTTTGGGAAATCAGACCCCTT | |
| | CCTTGTGTTCTACAGGAGCAATGAGCATCGCACGTTCACCATCTGCCACAAGACAGAG | |
| | GTTGTGAAAAACACGCTGAATCCTGTGTGGCAGCCCTTCAGCATCCCTGTGCGGGCTC | |
| | TGTGCAATGGAGACTATGACAGAACGGTGAAGATTGATGTGTACGACTGGGACCGGGA | |
| | TGGAAGCCACGATTTCATTGGTGAGTTCACCACCAGCTACCGGGAGCTGAGCAAGGCC | |
| | CAGAACCAGTTCACAGTATATGAGGTTCTTAACCCTCGGAAGAAATGTAAGAAGAAGA | |
| | AATATGTCAACTCAGGAACTGTGACGCTGCTCTCCTTCTCTGTGGACTCTGAATTCAC | |
| | TTTTGTTGATTACATCAAGGGAGGGACACAGCTCAACTTCACACTAGCCATTCACTTC | |
| | ACGGCTTCCAATGGGAATCCTCTGCAGCCTACCTCCCTGCACTACATGAGTCCCTACC | |
| | AGCTCAGCGCCTATGCCATGGCCCTCAAGGCAGTGGGAGAGATCATCCAGGACTATGA | |
| | CAGTGATAAGCTCTTCCCAGCTTATGGCTTTGGGGCCAAGCTGCCCCCAGAGGGACGG | |
| | ATCTCCCACCAGTTCCCCCTGAACAACAATGATGAGGACCCCAACTGTGCGGGCATCG | |
| | AGGGTGTGCTGGAGAGCTATTTCCAGAGCCTGCGCACAGTGCAGCTCTATGGGCCCAC | |
| | CTACTTTGCTCCTGTCATCAACCAAGCGGCCAGGGCTGCAGCCAAGATCTCTGATGGC | |
| | TCCCAGTACTATGTTCTGCTCATCATCACTGATGGGGTCATCTCTGACATGACGCAGA | |
| | CCAAGGAGGCCATCGTCAGCGCCTCCTCATTGCCCATGTCTATCATTATCGTCGGTGT | |
| | AGGACCAGCCATGTTTGAGGCAATGGAAGAGTTGGACGGTGATGATGTGCGCGTGTCC | |
| | TCTAGCGCACGCTACGCAGAGCGGGACATCGTTCAGTTCGTCCCATTCCGAGACTATG | |
| | TTGACCGGTCGGGGAACCAGCTGTTGAGCATGGCCCGACTGGCCAAGGATGTGCTGGC | |
| | CGAGATCCCGGAGCAGCTGCTGTCCTATATGCGCACCAGAGACATCCACCCTCGGCCC | |
| | CCACCCCCTGCCAACCCCAGCCCGATCCCAGCTCCAGAGCAGCCCTGAGGATTCCACA | |
| | TATCCAATGCCTCACAGTCTGCAAGCCTGCTCACCCACTGCTTCTGCTTTAAGCCAGA | |
| | GG | |
| | ORF Start: ATG at 11 | ORF Stop: TGA at 1670 |
| | SEQ ID NO:98 | 553 aa MW at 61835.3 kD |
| NOV23b, | MSLGGASERSVPATKIEITVSCRNLLDLDTFSKSDPMVVLYTQSRASQEWREFGRTEV | |
| CG57734-02 Protein | IDNTLNPDFVRKFVLDYFFEEKQNLRFDVYNvDSKTNISKPKDFLGQAFLALGEVIGG | |
| Sequence | QGSRVERTLTGVPGKKCGTILLTAEELSNCRDIATMQLCANKLDKKDFFGKSDPFLVF | |
| | YRSNEDGTFTICHKTEVVKNTLNPVWQPFSIPVRALCNGDYDRTVKIDVYDWDRDGSH | |
| | DFIGEFTTSYRELSKAQNQFTVYEVLNPRKKCKKKKYVNSGTVTLLSFSVDSEFTFVD | |
| | YIKGGTQLNFTVAIDFTASNGNPLQPTSLHYMSPYQLSAYAMALKAVGEIIQDYDSDK | |
| | LFPAYGFGAKLPPEGRISHQFPLNNDEDPNCAGIEGVLESYFQSLRTVQLYGPTYYFA | |
| | PVINQAARAAAKISDGSQYYVLLIITDCVISDMTQTKEAIVSASSLPMSIIIVGVGPA | |
| | MFEAMEELDGDDVRVSSRGRYAERDIVQFVPFRDYVDRSGNQVLSMARLAKDVLAEIP | |
| | EQLLSYMRTRDIQPRPPPPANPSPIPAPEQP | |
| | SEQ ID NO:99 | 1368 bp |
| NOV23c, | GGATCCATGTCTGTCGGCCGAGCCTCCGAGCGCAGCGTCCCGGCCACCAAGATTGAAA | |
| 198363601 DNA | TTACCGTGTCCTGCCGGAACCTGCTAGACCTTGATACCTTCTCCAAGTCCGACCCCAT | |
| Sequence | GGTGGTGCTTTACGCGCAGAGCCGGGCCAGCCAGGAGTGGCGGGAGTTCGGACGGACC | |
| | GAGGTGATTGATAACACGCTGAACCCAGACTTCGTGCGCAAATTCGTCCTCGACTATT | |
| | TCTTTGAGGAAAAGCAAAATCTGCGCTTCGATGTGTACAACGTCGACTCCAAAACCAA | |
| | CATCTCCAAACCGAAGGATTTCCTGGGACAAGCGTTCCTGGCCCTGGGAGAGGTGATT | |
| | GGAGGCCAGGGCAGCCGAGTAGAGCGAACCCTCACGGGTGTACCAGGCAAGAAGTGTG | |
| | GGACCATATTGCTGACTGCAGAAGAGCTTAGCAATTGTCGGGACATTGCCACCATCCA | |
| | GCTGTGTGCAAACAAGCTGGACAAGAAGGACTTCTTTGGGAAATCAGACCCCTTCCTT | |
| | GTGTTCTACAGGAGTAATGAGGATGGCACGTTCACCATCTGCCACAAGACAGAGGTTG | |
| | TGAAAAACACGCTGAATCCTGTGTGGCAGCCCTTCAGCATCCCTGTGCGGGCTCTGTG | |
| | CAATGGAGACTATGACAGAACGGTGAAGATTGATGTGTACGACTGGGACCGCGATGGA | |
| | AGCCACGATTTCATTGGTGAGTTCACCACCAGCTACCGGGAGCTGAGCAAGGCCCAGA | |
| | ACCAGTTCACAGTATATGAGGTTCTTAACCCTCGGAAGAAATGTAAGAAGGAGAAATA | |
| | TGTCAACTCAGGAACTGTGACGCTGCTCTCCTTCTCTGTGGACTCTGAATTCACTTTT | |
| | GTTGATTACATCAAGGGACGGACACAGCTGAACTTCACAGTAGCCATTGACTTCACGG | |
| | CTTCCAATGGGAATCCTCTGCAGCCTACCTCCCTGCACTACATGAGTCCCTACCAGCT | |
| | CAGCGCCTATGCCATGGCCCTCAAGGCAGTGGGAGAGATCATCCAGGACTATGACAGT | |
| | GATAAGCTCTTCCCAGCTTATGGCTTTGGCGCCAAGCTGCCCCCAGAGGGACGGATCT | |
| | CCCACCAGTTCCCCCTGAACAACAATGATGAGGACCCCAACTGTGCGGGCATCGAGGG | |
| | TGTGCTGGAGAGCTATTTCCAGAGCCTGCGCACAGTGCAGCTCTATGGGCCCACCTAC | |
| | TTTGCTCCTGTCATCAACCAAGTGCCCAGGGCTGCAGCCAACATCTCTGATGGCTCCC | |
| | AGTACTATGTTCTGCTCATCATCACTdATGGGGTCATCTCTGACATGACGCAGACCAA | |
| | GGAGGCCATCGTCAGCGCCTCCTCATTGCTCGAG | |
| | ORF Start: GGA at 1 | ORF Stop: SD at 1369 |
| | SEQ ID NO:100 | 456 aa MW at 50948.9 kD |

| | | |
|---|---|---|
| NOV23c, | GSMSVGGASERSVPATKIEITVSCRNLLDLDTFSKSDPMVVLYAQSRASQEWREFGRT | |
| 198363601 | Protein | EVIDNTLNPDFVRKFVLDYFFEEKQNLRFDVYNVDSKTNISKPKDFLGQAFLALGEVI |
| Sequence | GGQGSRVERTLTGVPGKKCGTILLTAEELSNCRDIATMQLCANKLDKKDFFGKSDPFL | |
| | VFYRSNEDGTFTICHKTEVVKNTLNPVWQPFSIPVRALCNGDYDRTVKIDVYDWDRDG | |
| | SHDFIGEFTTSYRELSKAQNQFTVYEVLNPRKKCKKEKYVNSGTVTLLSFSVDSEFTF | |
| | VDYIKGGTQLNFTVAIDFTASNGNPLQPTSLHYMSPYQLSAYAMALKAVGEIIQDYDS | |
| | DKLFPAYGFGAKLPPEGRISHQFPLNNNDEDPNCAGIEGVLESYFQSLRTVQLYGPTY | |
| | FAPVINQVARAAAKISDGSQYYVLLIITDGVISDMTQTKEAIVSASSLLE | |

Sequence comparison of the above protein sequences yields the following sequence relationships shown in Table 23B.

TABLE 23B
Comparison of NOV23a against NOV23b through NOV23c.

| | NOV23a Residues/Match | Identities/Similarities |
|---|---|---|
| Protein Sequence | Residues | for the Matched Region |
| NOV23b | 1 . . . 572 | 523/572 (91%) |
| | 1 . . . 535 | 525/572 (91%) |
| NOV23c | 1 . . . 489 | 438/489 (89%) |
| | 3 . . . 454 | 442/489 (89%) |

Further analysis of the NOV23a protein yielded the following properties shown in Table 23C.

TABLE 23C
Protein Sequence Properties NOV23a

| | |
|---|---|
| PSort | 0.8500 probability located in endoplasmic reticulum |
| analysis: | (membrane); 0.4400 probability located in plasma membrane; |
| | 0.3388 probability located in microbody |
| | (peroxisome); 0.3000 probability located in nucleus |
| SignalP | No Known Signal Sequence Indicated |
| analysis: | |

A search of the NOV23a protein against the Geneseq database, a proprietary database that contains sequences published in patents and patent publication, yielded several homologous proteins shown in Table 23D.

TABLE 23D
Geneseq Results for NOV23a

| | | NOV23a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAY97293 | Lipid associated protin (LIPAP) | 10 . . . 583 | 412/575 (71%) | 0.0 |
| | 3335404CD1 - Homo sapiens, 564 aa. | 20 . . . 556 | 475/575 (81%) | |
| | [WO200049043-A2, 24-AUG-2000] | | | |
| AAM39997 | Human polypeptide SEQ ID NO 3142 - | 76 . . . 579 | 248/511 (48%) | e−138 |
| | Homo sapiens, 548 aa. | 49 . . . 548 | 353/511 (68%) | |
| | [WO2001533 12-A1, 26-JUL-2001] | | | |
| AAB24231 | Human vesicle associated protein 10 | 76 . . . 579 | 248/511 (48%) | e−138 |
| | SEQ ID NO: 10 - Homo sapiens, 532 | 33 . . . 532 | 353/511 (68%) | |
| | aa. [WO200060082-A2, 12-OCT-2000] | | | |
| AAU19736 | Human novel extracellular matrix | 64 . . . 582 | 257/527 (48%) | e−137 |
| | protein, Seq ID No 386 - Homo | 25 . . . 534 | 351/527 (65%) | |
| | sapiens, 540 aa. [WO200155368-A1, | | | |
| | 02-AUG-2001] | | | |
| AAU19664 | Human novel extracellular matrix | 286 . . . 590 | 234/308 (75%) | e−133 |
| | protein, Seq ID No 314 - Homo | 20 . . . 327 | 268/308 (86%) | |
| | sapiens, 335 aa. [WO200155368-A1, | | | |
| | 02-AUG-2001] | | | |

In a BLAST search of public sequence databases, the NOV23a protein was found to have homology to the proteins shown in the BLASTP data in Table 23E.

TABLE 23E
Public BLASTP Results for NOV23a

| | | NOV23a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9HCH3 | Copine-like protein KIAA 1599 - Homo | 10 . . . 590 | 422/604 (69%) | 0.0 |
| | sapiens (Human), 593 aa. | 19 . . . 585 | 484/604 (79%) | |
| Q9DC53 | 1200003E11RIK PROTEIN - Mus | 10 . . . 583 | 411/575 (71%) | 0.0 |
| | musculus (Mouse), 577 aa. | 33 . . . 569 | 477/575 (82%) | |
| O75131 | Copine III - Homo sapiens (Human), | 64 . . . 582 | 257/527 (48%) | e−137 |
| | 537 aa. | 22 . . . 531 | 352/527 (66%) | |
| Q96A23 | CDNA FLJ31613 FIS,CLONE | 55 . . . 574 | 258/528 (48%) | e−135 |
| | NT2R12002958, MODERATELY | 30 . . . 543 | 347/528 (64%) | |
| | SIMILAR TO HOMO SAPIENS | | | |
| | COPINE VI PROTEIN (SIMILAR | | | |
| | TO RIKEN | | | |
| | CDNA 3632411M23 GENE) - Homo | | | |
| | sapiens (Human), 557 aa. | | | |
| Q99829 | Copine I - Homo sapiens (Human), | 68 . . . 588 | 250/528 (47%) | e−133 |
| | 537 aa. | 24 . . . 536 | 352/528 (66%) | |

PFam analysis indicates that the NOV23a protein contains the domains shown in the Table 23F.

TABLE 23F
Domain Analysis of NOV23a

| | NOV23a | Identities/ | |
|---|---|---|---|
| | Match | Similarities | Expect |
| Pfam Domain | Region | for the Matched Region | Value |
| C2: domain 1 of 1 | 191 . . . 276 | 32/99 (32%) | 6.5e−07 |
| | | 59/99 (60%) | |

### Example 24

The NOV24 clone was analyzed, and the nucleotide and polypeptide sequences are shown in Table 24A.

TABLE 24A
NOV24 Sequence Analysis

| | |
|---|---|
| SEQ ID NO: 101 | 1904 bp |

| | |
|---|---|
| NOV24a, | TTTTTTTTTGACACTGTAAAAGAAGTTTATTTCTTGTTCACATAACTGTCCAAGGCTG |
| CG59389-01 DNA | GTGTTCCTGGGCAACTTCCAGTTCTCTTCCATGCAATGATTCAAGGACACACAGTTCT |
| Sequence | TCCATCCTGTGACTCTACCATCCCCTAGAGCCCTCTACTGTAAGAGTTACCTAAAGCA |
| | TCTGTGATGGTCCAGGAGGCTTCTCAGGTGATCGGGCAGTGTCAGTCTTCAGCCACTA |
| | AGCCCAGAAGATCTGGGAAGAAGTCAATCAGAGAGCCTTGGGCCAGAGTTCCAGGGGC |
| | TCTGGGAGTGGCTGCCAGGTGAGCTGGACAGTCTGATTTTCAGTGGGGTCCACACAGA |
| | TGGGACGCGGCTTAGGAGGAATCCTGGGCTGCAGGCATTCCTTGGCCTGGTAGTCAGA |
| | TTTCTGGCACTTGTAGCAAGCTCCTGGCGGAGAAGGTTCTGGAGTAACGCCTGGCCGC |
| | TGCGGTTCAGGCATTTGGAAGTTCTTGTGTGCTGGACATCTGCCTGGGGTTTGTCTCA |
| | CAGTGGAGGTTACCTAACCAAACTCCTGTAAAACCACACCACCTATGCCTGTGATGGG |
| | GACTATTTGAATCTACAGTGCCCTCGGCATTCTACGATAAGTGTCCAATCGGCATTTT |
| | ATGGGCAAGATTACCAAATGTGTAGTTCCCAGAAGCCTGCCTCCCAGAGGGAAGACAG |
| | CTTAACCTGTGTGGCAGCCACCACCTTCCAGAAGGTGCTGGACGAATGCCAGAACCAG |
| | CGGGCCTGCCACCTCCTGGTCAATAGCCGTGTTTTTGGACCTGACCTTTGTCCAGGAA |
| | GCAGTAAATACCTCCTGGTCTCCTTTAAATGCCAACCTAATGAATTAAAAAACAAAAC |
| | CGTGTGTGAAGACCAGGAGCTGAAACTGCACTGCCATGAATCCAAGTTCCTCAACATC |
| | TACTCTGCGACCTACGGCAGGAGGACCCAGGAAAGGCACATCTGCTCCTCCAACGCAG |
| | AGCGGCTCCCCCCTTTCGATTGCTTGTCTTACTCAGCTTTGCAAGTCCTATCCCGAAG |
| | GTGCTATGGGAAGCAGAGATGCAAAATCATCGTCAACAATCACCATTTTGGAAGCCCC |
| | TGTTTGCCAGGCATGAAAAAATACCTCACTGTGACCTACGCATGTGTTCCCAAGAACA |
| | TACTCACACCGATTGATCCAGCCATTGCTAATCTAAAACCTTCTTTGAAGCAGAAAGA |
| | TGGTGAATATGGTATAAACTTTGACCCAAGCGGATCGAAGGTTCTGAGGAAAGATGGA |
| | ATTCTTGTTAGCAACTCTCTGGCAGCCTTTGCTTACATTAGAGCCCACCCGGAGAGAG |
| | CTGCCCTGCTGTTCGTCTCCAGTGTCTGCATCGGCCTGGCCCTCACACTGTGCGCCCT |
| | GGTCATCAGAGAGTCCTGTGCCAAGGACTTCCCCGACTTGCAGCTGGGGAGGGAGCAG |
| | CTGGTGCCAGGAAGTGACAAGGTCGAGCAGGACAGCGAGGATGAAGAAGACGAGGAGG |
| | ACCCCTCTGAGTCTGATTTCCCAGGGGAACTGTCGGGGTTCTGTAGGACTTCATATCC |
| | TATATACAGTTCCATAGAAGCTGCAGAGCTCGCAGAAAGGATTGAGCGCAGGGAGCAA |
| | ATCATTCAGGAAATATGGATGAACAGTGGTTTGGACACCTCGCTCCCAAGAAACATGG |
| | GCCAGTTCTACTGAAAACCACATGCATCTTGATGCGATCGCACTTTCTGAAGAAGGAA |
| | GGATCCCAAATGCCCCTCCAGTTCTGGTTCACCTGTACCTTCTATGAAGGAGAATTCG |
| | TCATGTCATTCAACACTCGTGAGGCCAGGAAGCTATTAAAGGGATGTTTCAAGCTGTT |
| | TCTAGCACATTCCAAATAAATGAGGAGGGAAGAAAAAAAAAAAAAAA |

| ORF Start: ATG at 656 | ORF Stop: TGA at 1694 | |
|---|---|---|
| SEQ ID NO: 102 | 346 aa | MW at 38793.6kD |

| | |
|---|---|
| NOV24a, | MCSSQKPASQREDSLTCVAATTFQKVLDECQNQRACHLLVNSRVFGPDLCPGSSKYLL |
| CG59389-01 Protein | VSFKCQPNELKNKTVCEDQELKLHCHESKFLNIYSATYGRRTQERDICSSKAERLPPF |
| Sequence | DCLSYSALQVLSRRCYGKQRCKIIVNNHHFGSPCLPGMKKYLTVTYACVPKNILTAID |
| | PAIANLKPSLKQKDGEYGINFDPSGSKVLRKDGILVSNSLAAFAYIRAHPERAALLFV |
| | SSVCIGLALTLCALVIRESCAKDFRDLQLGREQLVPGSDKVEEDSEDEEEEEDPSESD |
| | FPGELSGFCRTSYPIYSSIEAAELAERIERREQIIQEIWMNSGLDTSLPRNMGQFY |

| | |
|---|---|
| SEQ ID NO: 103 | 1802 bp |

| | |
|---|---|
| NOV24b, | TTTTTTTTTGACACTCTAAAAGAAGTTTATTTCTTGTTCACATAACTGTCCAAGGCTG |
| CG59389-02 DNA | GTGTTCCTGGGCAACTTCCAGTTCTCTTCCATGCAATGATTCAAGGACACAGAGTTCT |
| Sequence | TCCATCCTGTGACTCTACCATCCCCTAGAGCCCTCTACTGTAAGAGTTACCTAAAGCA |
| | TCTGTGATGGTCCAGGAGGCTTCTCAGGTGATCGGGCAGTGTCAGTCTTCAGCCACTA |
| | AGCCCAGAAGATCTGGGAAGAAGTCAATCAGAGAGCCTTGGGCCAGAGTTCCAGGGGC |
| | TCTGGGAGTCGCTGCCAGGTGAGCTGGACAGTCTGATTTTCAGTGGGGTCCACACACA |
| | TGGGACGCGGCTTAGGAGGAATCCTGGGCTGCAGGCATTCCTTGCCCTGCTAGTCAGA |
| | TTTCTGGCACTTGTAGCAAGCTCCTGGGGGAGAAGGTTCTGGAGTAACGCCTGGCCGC |
| | TGCGCTTCAGGCATTTGGAAGTTCTTGTGTGCTGGAGATGTGGCTGGGGTTTGTCTCA |
| | CAGTGGAGGTTACCTAACCAAACTCCTGTAAAACCACACCACCTATGCCTGTGATGGG |
| | GACTATTTGAATCTACAGTGCCCTCGGCATTCTACGATAAGTGTCCAATCGGCATTTT |
| | ATGGGCAAGATTACCAAATGTGTAGTTCCCAGAAGCCTGCCTCCCAGAGGGAAGACAG |
| | CTTAACCTGTGTGGCAGCCACCACCTTCCAGAAGGTGCTGGACGAATGCCAGAACCAG |
| | CGGGCCTGCCACCTCCTGGTCAATAGCCGTGTTTTTGGACCTGACCTTTGTCCAGGAA |
| | GCAGTAAATACCTCCTGGTCTCCTTTAAATGCCAACCTAATGAATTAAAAAACAAAAC |
| | CGTGTGTGAAGACCAGGAGCTGAAACTGCACTGCCATGAATCCAAGTTCCTCAACATC |
| | TACTCTGCGACCTACGGCAGGAGGACCCAGGAAAGGGACATCTGCTCCTCCAAGGCAG |
| | AGCGGCTCCCCCCTTTCGATTGCTTGTCTTACTCAGCTTTGCAAGTCCTATCCCGAAG |
| | GTGCTATGGGAAGCAGAGATGCAAAATCATCGTCAACAATCACCATTTTGGAAGCCCC |
| | TGTTTGCCAGGCGTGAAAAAATACCTCACTCTGACCTACGCATGTCGTATAAACTTCG |
| | ACCCAAGCGGATCGAAGGTTCTGAGGAAAGATGGAATTCTTGTTAGCAACTCTCTGGC |
| | AGCCTTTGCTTACATTAGAGCCCACCCGGAGAGAGCTGCCCTGCTGTTCGTGTCCAGT |
| | GTCTGCATCGGCCTGGCCCTCACACTGTGCGCCCTGGTCATCAGAGAGTCCTGTGCCA |
| | AGGACTTCCGCGACTTGCAGCTGGGGAGGGAGCAGCTGGTGCCAGGAAGTGACAAGGT |
| | CGAGGAGGACACCGAGCATGAAGAAGAGGAGGAGGACCCCTCTGAGTCTGATTTCCCA |
| | GGGGAACTGTCGCGGTTCTGTAGGACTTCATATCCTATATACAGTTCCATAGAAGCTG |
| | CAGAGCTCGCAGAAAGGATTCAGCGCAGGGAGCAAATCATTCAGGAAATATGGATGAA |
| | CAGTGGTTTGGACACCTCGCTCCCAAGAAACATGGGCCAGTTCTACTGAAAACCACAT |
| | GCATCTTGATGCGATCGCACTTTCTGAAGAAGCAAGGATCCCAAATGCCCCTCCAGTT |
| | CTGGTTCACCTGTACCTTCTATGAAGGAGAATTCGTCATGTCATTCAACACTCGTGAG |
| | GCCAGGAAGCTATTAAAGGGATGTTTCAAGCTGTTTCTAGCACAGGGGCTTCCAGCAT |
| | CCTG |

| ORF Start: ATG at 656 | ORF Stop: TGA at 1613 | |
|---|---|---|
| SEQ ID NO: 104 | 319 aa | MW at 35842.2 kD |

| | |
|---|---|
| NOV24b, | MCSSQKPASQREDSLTCVAATTFQKVLDECQNQRACHLLVNSRVFGPDLCPGSSKYLL |
| CG59389-02 Protein | VSFKCQPNELKNKTVCEDQELKLHCHESKFLNIYSATYGRRTQERDICSSKAERLPPF |
| Sequence | DCLSYSALQVLSRRCYGKQRCKIIVNNHHFGSPCLPGVKKYLTVTYACGINFDPSGSK |
| | VLRKDGILVSNSLAAFAYIRAHPERAALLFVSSVCIGLALTLCALVIRESCAKDFRDL |
| | QLGREQLVPGSDKVEEDSEDEEEEEDPSESDFPGELSGFCRTSYPIYSSIEAAELAER |
| | IERREQIIQEIWMNSGLDTSLPRNMGQFY |

| | |
|---|---|
| SEQ ID NO: 105 | 1326 bp |

| | |
|---|---|
| NOV24c, | TCCCCGCCATGTGACGCCGTCCTTAGCCCTGCGACCCCCAGCGCGTCCCGGGCCTGCG |
| CG59389-04 DNA | CCTCCGCCCCGCCGCGCAGCACGATGCTTCTGCCGGGACGCGCACGCCAACCGCCGAC |
| Sequence | GCCCCAGCCCGTGCAGCATCCCGGCCTCCGCCGGCAGGTAGAGCCGCCGGGGCAGCTC |
| | CTGCGCCTCTTCTACTGCACTGTCCTGGTCTGCTCCAAAGAGATCTCAGCGCTCACCG |
| | ACTTCTCTGGTTACCTAACCAAACTCCTGCAAAACCACACCACCTATGCCTGTGATGG |
| | GGACTATTTGAATCTACAGTGCCCTCGGCATTCTACGATAAGTGTCCAATCGGCATTT |
| | TATGGGCAAGATTACCAAATGTGTAGTTCCCAGAAGCCTGCCTCCCAGAGGGAAGACA |
| | GCTTAACCTGTGTGGCAGCCACCACCTTCCAGAAGGTGCTGGACGAATGCCAGAACCA |
| | GCGGGCCTGCCACCTCCTGGTCAATAGCCGTGTTTTTGGACCTGACCTTTGTCCAGGA |
| | AGCAGTAAATACCTCCTGGTCTCCTTTAAATGCCAACCTAATGAATTAAAAAACAAAA |
| | CCGTGTGTGAAGACCAGGAGCTGAAACTGCACTGCCATGAATCCAAGTTCCTCAACAT |
| | CTACTCTGCGACCTACGGCAGGAGGACCCAGGAAAGGGACATCTGCTCCTCCAAGGCA |
| | GAGCCGCTCCCCCCTTTCGATTGCTTGTCTTACTCAGCTTTGCAAGTCCTATCCCGAA |
| | GGTGCTATGGGAAGCAGAGATGCAAAATCATCGTCAACAATCACCATTTTGGAAGCCC |
| | CTGTTTGCCAGGCGTGAAAAAATACCTCACTGTGACCTACGCATGTGGTATAAACTTC |
| | GACCCAAGCGGATCGAAGGTTCTGAGGAAAGATGGAATTCTTGTTAGCAACTCTCTGG |
| | CAGCCTTTGCTTACATTAGAGCCCACCCGGAGAGAGCTGCCCTGCTGTTCGTGTCCAG |
| | TGTCTGCATCGGCCTGGCCCTCACACTGTGCGCCCTGGTCATCAGAGAGTCCTGTGCC |
| | AAGGACTTCCGCGACTTGCAGCTGGGGAGGGAGCAGCTGGTGCCAGGAAGTGACAAGG |
| | TCGAGGAGGACAGCGAGGATGAAGAAGAGGAGGAGCACCCCTCTGAGTCTGATTTCCC |
| | AGGGGAACTGTCGGGGTTCTGTAGGACTTCATATCCTATATACAGTTCCATAGAAGCT |
| | GCAGACCTCGCAGAAAGGATTGAGCGCAGGGAGCAAATCATTCAGGAAATATGGATGA |
| | ACAGTGGTTTGGACACCTCGCTCCCAAGAAACATGGGCCAGTTCTACTGA |

| ORF Start: ATG at 82 | ORF Stop: TGA at 1324 | |
|---|---|---|
| SEQ ID NO: 106 | 414 aa | MW at 46563.4 kD |

| | |
|---|---|
| NOV24c, | MLLPGRARQPPTPQPVQHPGLRRQVEPPCQLLRLFYCTVLVCSKEISALTDFSGYLTK |
| CG59389-04 Protein | LLQNHTTYACDGDYLNLQCPRHSTISVQSAFYGQDYQMCSSQKPASQREDSLTCVAAT |
| Sequence | TFQKVLDECQNQRACHLLVNSRVFGPDLCPGSSKYLLVSFKCQPNELKNKTVCEDQEL |
| | KLHCHESKFLNIYSATYGRRTQERDICSSKAERLPPFDCLSYSALQVLSRRCYGKQRC |
| | KIIVNNHHFGSPCLPGVKKYLTVTYACGINFDPSGSKVLRKDGILVSNSLAAFAYIRA |
| | HPERAALLFVSSVCIGLALTLCALVIRESCAKDFRDLQLGREQLVPGSDKVEEDSEDE |
| | EEEEDPSESDFPGELSGFCRTSYPIYSSIEAAELAERIERREQIIQEIWMNSGLDTSL |
| | PRNMGQFY |

| | |
|---|---|
| SEQ ID NO: 107 | 693 bp |

| | |
|---|---|
| NOV24d, | AAGCTTACCATGTGTAGTTCCCAGAGCCTGCCTCCCAGAGGGAAGACAGCTTAACCT |
| 174308481 DNA | GTGTGGCAGCCACCACCTTCCAGAAGGTGCTGGACGAATGCCAGAACCAGCGGGCCTG |
| Sequence | CCACCTCCTGGTCAATAGCCGTGTTTTTGGACCTGACCTTTGTCCAGGAAGCAGTAAA |
| | TACCTCCTGGTCTCCTTTAAATGCCAACCTAATGAATTAAAAAACAAAACCGTGTGTG |
| | AAGACCAGGAGCTGAAACTGCACTGCCATGAATCCAAGTTCCTCAACATCTACTCTGC |
| | GACCTACGGCAGGAGGACCCAGGAAAGGGACATCTGCTCCTCCAAGGCAGGGCGGCTC |
| | CCCCCTTTCGATTGCTTGTCTTACTCAGCTTTGCAAGTCCTATCCCGAAGGTGCTATG |
| | GGAAGCAGAGATGCAAAATCATCGTCAACAATCACCATTTTGGAAGCCCCTGTTTGCC |
| | AGGCGTGAAAAAATACCTCACTGTGACCTACGCATGTGTTCCCAAGAACATACTCACA |
| | GCGATTGATCCAGCCATTGCTAATCTAAAACCTTCTTTGAAGCAGAAAGATGGTGAAT |
| | ATGGTATAAACTTCCACCCAAGCGGATCGAAGGTTCTGAGGAAAGATGGAATTCTTGT |
| | TAGCAACTCTCTGGCAGCCTTTGCTTACATTAGAGCCCACCCGGAGAGACTCGAG |

| ORF Start: AAG at 1 | ORF Stop: 37 at 694 | |
|---|---|---|
| SEQ ID NO: 108 | 231 aa | MW at 25807.5 kD |

| | |
|---|---|
| NOV24d, | KLTMCSSQKPASQREDSLTCVAATTFQKVLDECQNQRACHLLVNSRVFGPDLCPCSSK |
| 174308481 Protein | YLLVSFKCQPNELKNKTVCEDQELKLHCHESKFLNIYSATYGRRTQERDICSSKAGRL |
| Sequence | PPFDCLSYSALQVLSRRCYCKQRCKIIVNNHHFGSPCLPGVKKYLTVTYACVPKNILT |
| | AIDPAIANLKPSLKQKDGEYGINFDPSGSKVLRKDGILVSNSLAAFAYIRAHPERLE |

| | |
|---|---|
| SEQ ID NO: 109 | 693 bp |

| | |
|---|---|
| NOV24e, | AAGCTTACCATGTGTAGTTCCCAGAAGCCTGCCTCCCAGAGGCAAGACAGCTTAACCT |
| 174308497 DNA | GTGTGGCAGCCACCACCTTCCAGAAGGTGCTGGACGAATGCCTGAACCAGCGGGCCTG |
| Sequence | CCACCTCCTGGTCAATAGCCGTGTTTTTGGACCTGACCTTTGTCCAGGAACCAGTAAA |
| | TACCTCCTGGTCTCCTTTAAATGCCAACCTAATGAATTAAAAAACAAAACCGTGTGTG |
| | AAGACCAGGAGCTGAAACTCCACTGCCATGAATCCAAGTTCCTCAACATCTACTCTGC |
| | GACCTACGGCAGGAGGACCCAGGAAAGGGACATCTGCTCCTCCAAGGCAGAGCGGCTC |
| | CCCCCTTTCGATTGCTTGTCTTACTCAGCTTTGCAAGTCCTATCCCCAAGGTGCTATG |
| | GGAAGCAGAGATGCAAAATCATCGTCAACAATCACCATTTTGGAAGCCCCTGTTTGCC |
| | AGGCGTGAAAAAATACCTCACTGTCACCTACGCATGTGTTCCCAAGAACATACTCACA |
| | GCGATTGATCCAGCCATTGCTAATCTAAAACCTTCTTTGAAGCAGAAAGATGGTGAAT |
| | ATGGTATAAACTTCGACCCAAGCCGATCGAAGGTTCTGAGGAAAGATGGAATTCTTGT |
| | TAGCAACTCTCTGGCAGCCTTTGCTTACATTAGAGCCCACCCGGAGACACTCGAG |

| ORF Start: AAG at 1 | ORF Stop: 37 at 694 | |
|---|---|---|
| SEQ ID NO: 110 | 231 aa | MW at 25864.5 kD |

| | |
|---|---|
| NOV24e, | KLTMCSSQKPASQREDSLTCVAATTFQKVLDECLNQRACHLLVNSRVFGPDLCPGSSK |
| 174308497 Protein | YLLVSFKCQPNELKNKTVCEDQELKLHCHESKFLNIYSATYGRRTQERDICSSKAERL |
| Sequence | PPFDCLSYSALQVLSRRCYGKQRCKIIVNNHHFGSPCLPGVKKYLTVTYACVPKNILT |
| | AIDPAIANLKPSLKQKDCEYGINFDPSGSKVLRKDGILVSNSLAAFAYIRAHPERLE |

| | |
|---|---|
| SEQ ID NO: 111 | 693 bp |

| | |
|---|---|
| NOV24f, | AAGCTTACCATGTGTAGTTCCCAGAAGCCTGCCTCCCAGAGGGAAGACAGCTTAACCT |
| 174308507 DNA | GTCTGGCAGCCACCACCTTCCAGAAGGTGCTGGACGAATGCCAGAACCAGCGGGCCTG |
| Sequence | CCACCTCCTGGTCAATAGCCGTGTTTTTGGACCTGACCTTTGTCCAGGAAGCAGTAAA |
| | TACCTCCTGGTCTCCTTTAAATGCCAACCTAATGAATTAAAAAACAAAACCGTGTGTG |
| | AAGACCAGGAGCTGAAACTGCACTGCCATGAATCCAAGTTCCTCAACATCTACTCTGC |
| | GACCTACGGCAGGAGGACCCAGGAAAGGGACATCTGCTCCTCCAAGCCAGACCGGCTC |
| | CCCCCTTTCGATTGCTTGTCTTACTCAGCTTTGCAAGTCCTATCCCGAAGGTGCTATG |
| | GGAAGCAGAGATGCAAAATCATCGTCAACAATCACCATTTTGGAAGCCCCTGTTTGCC |
| | AGGCGTGAAAAAATACCTCACTGTGACCTACGCATGTGTTCCCAAGAACATACTCACA |
| | GCGATTGATCCAGCCATTGCTAATCTAAAACCTTCTTTGAAGCAGAAAGATGGTGAAT |
| | ATGGTATAAACTTCGACCCAAGCGGATCGAAGGTTCTGAGGAAAGATGGAATTCTTGT |
| | TAGCAACTCTCTGGCAGCCTTTGCTTACATTAGAGCCCACCCGGACAGACTCGAG |

| ORF Start: AAG at 1 | ORF Stop: 38 at 694 | |
|---|---|---|
| SEQ ID NO: 112 | 231 aa | MW at 25879.5 kD |

| | |
|---|---|
| NOV24f, | KLTMCSSQKPASQREDSLTCVAATTFQKVLDECQNQRACHLLVNSRVFGPDLCPGSSK |
| 174308507 Protein | YLLVSFKCQPNELKNKTVCEDQELKLHCHESKFLNIYSATYGRRTQERDICSSKAERL |
| Sequence | PPFDCLSYSALQVLSRRCYGKQRCKIIVNNHHFGSPCLPGVKKYLTVTYACVPKNILT |
| | AIDPAIANLKPSLKQKDGEYGINFDPSGSKVLRKDGILVSNSLAAFAYIRAHPERLE |

| | |
|---|---|
| SEQ ID NO: 113 | 690 bp |

| | |
|---|---|
| NOV24g, | AAGCTTACCATGTGTAGTTCCCAGAAGCCTGCCTCCCAGAGGGAAGACACCTTAACCT |
| 174308517 DNA | GTGTGGCAGCCACCACCTTCCAGAAGGTGCTGGACGAATGCCAGAACCAGCGGGCCTG |
| Sequence | CCACCTCCTGGTCAATAGCCGTGTTTTTGGACCTGACCTTTGTCCAGGAAGCAGTAAA |
| | TACCTCCTCGTCTCCTTTAAATGCCAACCTAAATTAAAAAACAAAACCGTGTGTGAAG |
| | ACCAGGAGCTGAAACTGCACTGCCATGAATCCAAGTTCCTCAACATCTACTCTGCGAC |
| | CTACGGCAGGAGGACCCAGGAAAGGCACATCTGCTCCTCCAAGGCAGAGCGGCTCCCC |
| | CCTTTCGATTGCTTGTCTTACTCAGCTTTGCAAGTCCTATCCCGAACGTGCTATGGGA |
| | AGCAGAGATGCAAAATCATCGTCAACAATCACCATTTTGGAAGCCCCTGTTTCCCAGG |
| | CGTGAAAAAATACCTCACTGTGACCTACGCATGTGTTCCCAAGAACATACTCACAGCG |
| | ATTGATCCAGCCATTGCTAATCTAAAACCTTCTTTGAAGCAGAAAGATGGTGAATATG |
| | GTATAAACTTCGACCCAAGCGGATCGAAGGTTCTGAGCAAAGATGGAATTCTTGTTAC |
| | CAACTCTCTGGCAGCCTTTGCTTACATTAGAGCCCACCCGGAGAGACTCGAG |

| ORF Start: AAG at 1 | ORF Stop: e− at 691 | |
|---|---|---|
| SEQ ID NO: 114 | 230 aa | MW at 25764.5 kD |

| | |
|---|---|
| NOV24g, | KLTMCSSQKPASQREDSLTCVAATTFQKVLDECQNQRACHLLVNSRVFCPDLCPGSSK |
| 174308517 Protein | YLLVSFKCQPKLKNKTVCEDQELKLHCRESKFLNIYSATYGRRTQERDICSSKAERLP |
| Sequence | PFDCLSYSALQVLSRRCYGKQRCKIIVNNHHFGSPCLPGVKKYLTVTYACVPKNILTA |
| | IDPAIANLKPSLKQKDGEYGINFDPSGSKVLRKDGILVSNSLAAFAYIRAHPERLE |

| | |
|---|---|
| SEQ ID NO: 115 | 693 bp |

| | |
|---|---|
| NOV24h, | AAGCTTACCATGTGTAGTTCCCAGAAGCCTGCCTCCCAGAGCGAAGACAGCTTAACCT |
| 174308525 DNA | GTGTGGCACCCACCACCCTCCAGAAGGTGCTGGACGAATGCCAGAACCAGCGGGCCTG |
| Sequence | CCACCTCCTGGTCAATAGCCGTGTTTTTGGACCTGACCTTTGTCCAGGAAGCAGTAAA |
| | TACCTCCTGGTCTCCTTTAAATGCCAACCTAATGAATTAAAAAACAAAACCGTGTGTG |
| | AAGACCAGGAGCTGAAACTGCACTGCCATGAATCCAAGTTCCTCAACATCTACTCTGC |
| | GACCTACGGCAGGAGGACCCAGGAAAGCGACGTCTGCTCCTCCAAGGCAGAGCGGCTC |
| | CCCCCTTTCGATTGCTTGTCTTACTCAGCTTTGCAAGTCCTATCCCGAAGGTGCTATG |
| | GGAAGCAGAGATGCAAAATCATCGTCAACAATCACCATTTTGGAAGCCCCTGTTTGCC |
| | AGGCGTGAAAAAATACCTCACTGTGACCTACGCATGTGTTCCCAAGAACATACTCACA |
| | GCCATTGATCCAGCCATTGCTAATCTAAAACCTTCTTTGAAGCAGAAAGATGGTGAAT |
| | ATGGTATAAACTTCGACCCAAGCGGATCGAAGGTTCTGAGGAAAGATGGAATTCTTGT |
| | TAGCAACTCTCTGGCAGCCTTTGCTTACATTAGAGCCCACCCGGAGAGACTCGAG |

| ORF Start: AAG at 1 | ORF Stop: 37 at 694 | |
|---|---|---|
| SEQ ID NO: 116 | 231 aa | MW at 25831.5 kD |

| | |
|---|---|
| NOV24h, | KLTMCSSQKPASQREDSLTCVAATTLQKVLDECQNQRACHLLVNSRVFGPDLCPGSSK |
| 174308525 Protein | YLLVSFKCQPNELKNKTVCEDQELKLHCHESKFLNIYSATYGRRTQERDVCSSKAERL |
| Sequence | PPFDCLSYSALQVLSRRCYGKQRCKIIVNNHHFGSPCLPGVKKYLTVTYACVPKNILT |
| | AIDPAIANLKPSLKQKDGEYGINFDPSGSKVLRKDGILVSNSLAAFAYIRAHPERLE |

Sequence comparison of the above protein sequences yields the following sequence relationships shown in Table 24B.

TABLE 24B
Comparison of NOV24a against NOV24b through NOV24h.

| | NOV24a Residues/ | Identities/Similarities |
|---|---|---|
| Protein Sequence | Match Residues | for the Matched Region |
| NOV24b | 1 . . . 346 | 281/346 (81%) |
| | 1 . . . 319 | 282/346 (81%) |
| NOV24c | 1 . . . 346 | 281/346 (81%) |
| | 96 . . . 414 | 282/346 (81%) |
| NOV24d | 1 . . . 226 | 224/226 (99%) |
| | 4 . . . 229 | 225/226 (99%) |
| NOV24e | 1 . . . 226 | 224/226 (99%) |
| | 4 . . . 229 | 225/226 (99%) |
| NOV24f | 1 . . . 226 | 225/226 (99%) |
| | 4 . . . 229 | 226/226 (99%) |
| NOV24g | 1 . . . 226 | 223/226 (98%) |
| | 4 . . . 228 | 225/226 (98%) |
| NOV24h | 1 . . . 226 | 223/226 (98%) |
| | 4 . . . 229 | 225/226 (98%) |

Further analysis of the NOV24a protein yielded the following properties shown in Table 24C.

TABLE 24C
Protein Sequence Properties NOV24a

| | |
|---|---|
| PSort | 0.7419 probability located in mitochondrial inner membrane; |
| analysis: | 0.4400 probability located in plasma membrane; 0.2000 |
| | probability located in endoplasmic reticulum |
| | (membrane); 0.1072 probability located in mitochondrial |
| | matrix space |
| SignalP | No Known Signal Sequence Indicated |
| analysis: | |

A search of the NOV24a protein against the Geneseq database, a proprietary database that contains sequences published in patents and patent publication, yielded several homologous proteins shown in Table 24D.

TABLE 24D
Geneseq Results for NOV24a

| | | NOV24a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| AAU12284 | Human PRO5993 polypeptide | 1 . . . 346 | 345/346 | (99%) | 0.0 |
| | sequence - Homo sapiens, 441 aa. | 96 . . . 441 | 346/346 | (99%) | |
| | [WO200140466-A2, 07-JUN-2001] | | | | |
| AAB68888 | Human RECAP polypeptide, SEQ ID | 1 . . . 346 | 345/346 | (99%) | 0.0 |
| | [WO200107612-A2, 01-FEB-2001] | 96 . . . 441 | 346/346 | (99%) | |
| AAB82854 | Human P2Y-like GPCR polypeptide - | 50 . . . 95 | 46/46 | (100%) | 2e−21 |
| | Homo sapiens, 158 aa. | 10 . . . 55 | 46/46 | (100%) | |
| | [WO200168842-A2, 20-SEP-2001] | | | | |
| AAB95410 | Human protein sequence SEQ ID | 209 . . . 346 | 64/144 | (44%) | 9e−20 |
| | NO: 17796 - Homo sapiens, 152 aa. | 16 . . . 152 | 89/144 | (61%) | |
| | [EP1074617-A2, 07-FEB-2001] | | | | |
| AAB93029 | Human protein sequence SEQ ID | 204 . . . 329 | 54/133 | (40%) | 7e−16 |
| | NO: 11799 - Homo sapiens, 165 aa. | 5 . . . 136 | 78/133 | (58%) | |
| | [EP1074617-A2, 07-FEB-2001] | | | | |

In a BLAST search of public sequence databases, the NOV24a protein was found to have homology to the proteins shown in the BLASTP data in Table 24E.

TABLE 24E
Public BLASTP Results for NOV24a

| | | NOV24a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P58658 | Protein C21 orf63 precursor (Protein | 1 . . . 346 | 345/346 (99%) | 0.0 |
| | PRED34) (SUE21) - Homo sapiens | 96 . . . 441 | 346/346 (99%) | |
| | (Human), 441 aa. | | | |
| Q9D4L3 | 4931408A02RIK PROTEIN - Mus | 1 . . . 346 | 294/346 (84%) | e−175 |
| | musculus (Mouse), 345 aa. | 1 . . . 345 | 322/346 (92%) | |
| P58659 | Protein C21 orf63 homolog precursor - | 1 . . . 346 | 294/346 (84%) | e−175 |
| | Mus musculus (Mouse), 440 aa. | 96 . . . 440 | 322/346 (92%) | |
| Q9H8M9 | CDNA FLJ13391 FIS, CLONE | 209 . . . 346 | 64/144 (44%) | 3e−19 |
| | PLACE1001241 (HYPOTHETICAL | 16 . . . 152 | 89/144 (61%) | |
| | 17.5 KDA PROTEIN) - Homo sapiens | | | |
| | (Human), 152 aa. | | | |
| Q9HC41 | HYPOTHETICAL 17.4 KDA | 209 . . . 346 | 64/144 (44%) | 3e−19 |
| | PROTEIN - Homo sapiens (Human), | 16 . . . 152 | 89/144 (61%) | |
| | 152 aa. | | | |

PFam analysis indicates that the NOV24a protein contains the domains shown in the Table 24F.

TABLE 24F
Domain Analysis of NOV24a

| | NOV24a | Identities/ | |
|---|---|---|---|
| | Match | for the Matched | Expect |
| Pfam Domain | Region | Region | Value |
| Gal_Lectin: domain 1 of 2 | 2 . . . 63 | 23/93 (25%) | 0.01 |
| | | 42/93 (45%) | |
| Gal_Lectin: domain 2 of 2 | 81 . . . 164 | 28/94 (30%) | 4.6e−13 |
| | | 52/94 (55%) | |

### Example 25

The NOV25 clone was analyzed, and the nucleotide and polypeptide sequences are shown in Table 25A.

TABLE 25A
NOV25 Sequence Analysis

| | |
|---|---|
| SEQ ID NO: 117 | 4517 bp |

| | |
|---|---|
| NOV25a, | GGCCCTCGCCGCCCGCGGCGCCCCGAGCGCTTTGTGAGCAGATGCGGAGCCGAGTGGA |
| CG59885-01 DNA | GGGCGCGAGCCAGATGCGGGGCGACAGCTGACTTGCTGAGAGGAGGCGGGGAGGCGCG |
| Sequence | GAGCGCGCGTGTGGTCCTTGCGCCGCTGACTTCTCCACTGGTTCCTGGGCACCGAAAG |
| | ATAAACCTCTCATAATGAAGGCCCCCGCTGTGCTTGCACCTGGCATCCTCCTGCTCCT |
| | GTTTACCTTGGTGCAGAGGAGCAATGGGGAGTGTAAAGAGGCACTAGCAAAGTCCGAG |
| | ATGAATGTGAATATGAAGTATCAGCTTCCCAACTTCACCGCGGAAACACCCATCCAGA |
| | ATGTCATTCTACATGAGCATCACATTTTCCTTGGTGCCACTAACTACATTTATGTTTT |
| | AAATGAGGAAGACCTTCAGAAGGTTGCTGAGTACAAGACTGGGCCTGTGCTGGAACAC |
| | CCAGATTGTTTCCCATGTCAGGACTGCAGCAGCAAAGCCAATTTATCAGGAGGTGTTT |
| | GGAAAGATAACATCAACATGGCTCTAGTTGTCGACACCTACTATGATGATCAACTCAT |
| | TAGCTGTGGCAGCGTCAACAGAGGGACCTGCCAGCGACATGTCTTTCCCCACAATCAT |
| | ACTGCTGACATACAGTCGGAGGTTCACTGCATATTCTCCCCACAGATAGAAGAGCCCA |
| | GCCAGTGTCCTGACTGTGTGGTGAGCGCCCTGGGAGCCAAAGTCCTTTCATCTGTAAA |
| | GGACCGGTTCATCAACTTCTTTGTAGGCAATACCATAAATTCTTCTTATTTCCCAGAT |
| | CATCCATTGCATTCGATATCAGTGAGAACGCTAAAGGAAACGAAAGATGGTTTTATGT |
| | TTTTGACGGACCAGTCCTACATTGATGTTTTACCTGAGTTCAGAGATTCTTACCCCAT |
| | TAAGTATGTCCATGCCTTTGAAAGCAACAATTTTATTTACTTCTTGACGGTCCAAAGG |
| | GAAACTCTAGATGCTCAGACTTTTCACACAAGAATAATCAGGTTCTGTTCCATAAACT |
| | CTGGATTGCATTCCTACATGGAAATGCCTCTGGAGTGTATTCTCACAGAAAAGAGAAA |
| | AAAGAGATCCACAAAGAAGGAAGTGTTTAATATACTTCAGGCTGCGTATGTCAGCAAG |
| | CCTGGGGCCCAGCTTGCTAGACAAATAGGAGCCAGCCTGAATGATGACATTCTTTTCG |
| | GGGTGTTCGCACAAAGCAAGCCAGATTCTGCCGAACCAATGGATCGATCTGCCATGTG |
| | TGCATTCCCTATCAAATATGTCAACGACTTCTTCAACAAGATCGTCAACAAAAACAAT |
| | GTGAGATGTCTCCAGCATTTTTACGGACCCAATCATGAGCACTGCTTTAATAGGACAC |
| | TTCTGAGAAATTCATCAGGCTGTGAAGCGCGCCGTGATGAATATCGAACAGAGTTTAC |
| | CACAGCTTTGCAGCGCGTTGACTTATTCATCGCTCAATTCAGCGAAGTCCTCTTAACA |
| | TCTATATCCACCTTCATTAAAGGAGACCTCACCATAGCTAATCTTGGGACATCAGAGG |
| | GTCGCTTCATGCAGGTTGTGGTTTCTCGATCAGGACCATCAACCCCTCATGTGAATTT |
| | TCTCCTGGACTCCCATCCAGTGTCTCCAGAAGTGATTGTGGAGCATACATTAAACCAA |
| | AATGGCTACACACTGGTTATCACTGGGAAGAAGATCACGAAGATCCCATTGAATGGCT |
| | TGGGCTGCAGACATTTCCAGTCCTGCAGTCAATGCCTCTCTGCCCCACCCTTTGTTCA |
| | GTGTGGCTGGTGCCACGACAAATGTGTOCGATCGCAGGAATGCCTGAGCGGGACATGG |
| | ACTCAACAGATCTGTCTGCCTGCAATCTACAAGGTTTTCCCAAATAGTGCACCCCTTG |
| | AAGGACGGACAAGGCTGACCATATGTGGCTGGGACTTTGGATTTCGGAGGAATAATAA |
| | ATTTGATTTAAAGAAAACTAGAGTTCTCCTTGGAAATGAGAGCTGCACCTTGACTTTA |
| | AGTGAGAGCACGATGAATACATTGAAATGCACAGTTGGTCCTGCCATGAATAACCATT |
| | TCAATATGTCCATAATTATTTCAAATGGCCACGGGACAACACAATACAGTACATTCTC |
| | CTATGTGGATCCTGTAATAACAAGTATTTCGCCGAAATACGGTCCTATGGCTGGTGGC |
| | ACTTTACTTACTTTAACTGGAAATTACCTAAACAGTGGGAATTCTAGACACATTTCAA |
| | TTGGTGGAAAAACATGTACTTTAAAAAGTGTGTCAAACAGTATTCTTGAATGTTATAC |
| | CCCAGCCCAAACCATTTCAACTGAGTTTGCTGTTAAATTGAAAATTGACTTAGCCAAC |
| | CGACAGACAAGCATCTTCAGTTACCGTGAAGATCCCATTGTCTATGAAATTCATCCAA |
| | CCAAATCTTTTATTAGTACTTGGTGGAAAGAACCTCTCAACATTGTCAGTTTTCTATT |
| | TTGCTTTGCCAGTGGTGGGAGCACAATAACAGGTGTTGGGAAAAACCTGAATTCAGTT |
| | AGTGTCCCGAGAATGGTCATAAATGTGCATGAAGCAGGAACGAACTTTACAGTGGCAT |
| | GTCAACATCGCTCTAATTCAGAGATAATCTGTTGTACCACTCCTTCCCTGCAACAGCT |
| | GAATCTGCAACTCCCCCTGAAAACCAAAGCCTTTTTCATGTTAGATGGGATCCTTTCC |
| | AAATACTTTGATCTCATTTATGTACATAATCCTGTGTTTAAGCCTTTTGAAAAGCCAG |
| | TGATGATCTCAATGGGCAATGAAAATGTACTGGAAATTAAGGGAAATGATATTGACCC |
| | TGAAGCAGTTAAAGGTGAAGTGTTAAAAGTTGGAAATAAGAGCTGTGAGAATATACAC |
| | TTACATTCTGAAGCCGTTTTATGCACGGTCCCCAATGACCTGCTGAAATTGAACAGCG |
| | AGCTAAATATAGAGTGGAAGCAAGCAATTTCTTCAACCGTCCTTGGAAAAGTAATAGT |
| | TCAACCAGATCAGAATTTCACAGGATTGATTGCTGGTGTTGTCTCAATATCAACAGCA |
| | CTGTTATTACTACTTGGGTTTTTCCTGTGGCTGAAAAAGAGAAAGCAAATTAAAGATC |
| | TGGGCAGTGAATTAGTTCGCTACGATGCAAGAGTACACACTCCTCATTTGGATAGGCT |
| | TGTAAGTGCCCGAAGTGTAAGCCCAACTACAGAAATGGTTTCAAATGAATCTGTAGAC |
| | TACCGAGCTACTTTTCCAGAAGATCAGTTTCCTAATTCATCTCAGAACGGTTCATGCC |
| | GACAAGTGCAGTATCCTCTGACAGACATGTCCCCCATCCTAACTAGTGGGGACTCTGA |
| | TATATCCAGTCCATTACTGCAAAATACTGTCCACATTGACCTCAGTGCTCTAAATCCA |
| | GAGCTGGTCCAGGCAGTGCAGCATGTAGTGATTGGGCCCAGTAGCCTGATTGTGCATT |
| | TCAATCAAGTCATAGGAAGAGGGCATTTTGGTTGTGTATATCATGGGACTTTGTTGGA |
| | CAATGATGGCAAGAAAATTCACTGTGCTGTGAAATCCTTGAACAGAATCACTGACATA |
| | GGAGAAGTTTCCCAATTTCTGACCGAGGGAATCATCATGAAAGATTTTAGTCATCCCA |
| | ATGTCCTCTCGCTCCTGGGAATCTGCCTGCGAAGTGAAGGGTCTCCGCTGGTGGTCCT |
| | ACCATACATGAAACATGGAGATCTTCGAAATTTCATTCGAAATGAGACTCATAATCCA |
| | ACTGTAAAAGATCTTATTGGCTTTGGTCTTCAAGTAGCCAAAGGCATGAAATATCTTG |
| | CAAGCAAAAAGTTTGTCCACAGAGACTTGGCTGCAAGAAACTGTATGCTGGATGAAAA |
| | ATTCACAGTCAAGGTTGCTGATTTTGGTCTTGCCAGAGACATGTATGATAAAGAATAC |
| | TATAGTGTACACAACAAAACAGGTGCAAAGCTGCCAGTGAAGTGGATGGCTTTGGAAA |
| | GTCTGCAAACTCAAAAGTTTACCACCAAGTCAGATGTGTGGTCCTTTGGCGTGCTCCT |
| | CTGGGAGCTGATGACAAGAGGAGCCCCACCTTATCCTGACGTAAACACCTTTGATATA |
| | ACTGTTTACTTGTTGCAAGGGAGAAGACTCCTACAACCCGAATACTGCCCAGACCCCT |
| | TATATGAAGTAATGCTAAAATGCTGGCACCCTAAAGCCGAAATGCGCCCATCCTTTTC |
| | TGAACTGGTGTCCCGGATATCAGCGATCTTCTCTACTTTCATTGGGGAGCACTATGTC |
| | CATGTGAACGCTACTTATGTGAACGTAAAATGTGTCGCTCCGTATCCTTCTCTGTTGT |
| | CATCAGAAGATAACGCTGATGATGAGGTGGACACACGACCAGCCTCCTTCTGGGAGAC |
| | ATCATAGTGCTAGTACTATGTCAAAGCAACAGTCCACACTTTGTCCAATGGTTTTTTC |
| | ACTGCCTGACCTTTAAAAGGCCATCGATATTCTTTGCTCTTGCCAAAATTG |

| ORF Start: ATG at 189 | ORF Stop: TAG at 4413 | |
|---|---|---|
| SEQ ID NO: 118 | 1408 aa | MW at 157710.2 kD |

| | |
|---|---|
| NOV25a, | MKAPAVLAPGILVLLFTLVQRSNGECKEALAKSEMNVNMKYQLPNFTAETPIQNVILH |
| CG59885-01 Protein | EHHIFLGATNYIYVLNEEDLQKVAEYKTGPVLEHPDCFPCQDCSSKANLSGGVWKDNI |
| Sequence | NMALVVDTYYDDQLISCGSVNRGTCQRHVFPHNHTADIQSEVHCIFSPQIEEPSQCPD |
| | CVVSALGAKVLSSVKDRFINFFVGNTINSSYFPDHPLHSISVRRLKETKDGFMFLTDQ |
| | SYIDVLPEFRDSYPIKYVHAFESNNFIYFLTVQRETLDAQTFHTRIIRFCSINSGLHS |
| | YMEMPLECILTEKRKKRSTKKEVFNILQAAYVSKPGAQLARQIGASLNDDILFGVFAQ |
| | SKPDSAEPMDRSAMCAFPIKYVNDFFNKIVNKNNVRCLQHFYGPNHEHCFNRTLLRNS |
| | SGCEARRDEYRTEFTTALQRVDLFMGQFSEVLLTSISTFIKGDLTIANLGTSEGRFMQ |
| | VVVSRSGPSTPHVNFLLDSHPVSPEVIVEHTLNQNGYTLVITGKKITKIPLNGLGCRH |
| | FQSCSQCLSAPPFVQCGWCHDKCVRSEECLSGTWTQQICLPAIYKVFPNSAPLBGGTR |
| | LTICGWDFGFRRNNKFDLKKTRVLLGNESCTLTLSESTMNTLKCTVGPAMNKHFNMSI |
| | IISNGHGTTQYSTFSYVDPVITSISPKYGPMAGGTLLTLTGNYLNSGNSRHISIGGKT |
| | CTLKSVSNSILECYTPAQTISTEFAVKLKIDLANRETSIFSYREDPIVYEIHPTKSFI |
| | STWWKEPLNIVSFLFCFASGGSTITGVGKNLNSVSVPRMVINVHEAGRNFTVACQHRS |
| | NSEIICCTTPSLQQLNLQLPLKTKAFFMLDGILSKYFDLIYVHNPVFKPFEKPVMISM |
| | GNENVLEIKGNDIDPEAVKGEVLKVGNKSCENIHLHSEAVLCTVPNDLLKLNSELNIE |
| | WKQAISSTVLGKVIVQPDQNFTGLIAGVVSISTALLLLLGFFLWLKKRKQIKDLGSEL |
| | VRYDAPVHTPHLDRLVSARSVSPTTEMVSNESVDYRATFPEDQFPNSSQNGSCRQVQY |
| | PLTDMSPILTSGDSDISSPLLQNTVHIDLSALNPELVQAVQHVVIGPSSLIVHFNEVI |
| | GRGHFGCVYHGTLLDNDGKKIHCAVKSLNRITDIGEVSQFLTEGIIMKDFSHPNVLSL |
| | LGICLRSEGSPLVVLPYMKHGDLRNFIRNETHNPTVKDLIGFGLQVAKGMKYLASKKF |
| | VHRDLAARNCMLDEKFTVKVADFGLARDMYDKEYYSVHNKTGAKLPVKWMALESLQTQ |
| | KFTTKSDVWSFGVLLWELMTRGAPPYPDVNTFDITVYLLQGRRLLQPEYCPDPLYEVM |
| | LKCWHPKAEMRPSFSELVSRISAIFSTFIGEHYVHVNATYVNVKCVAPYPSLLSSEDN |
| | ADDEVDTRPASFWETS |

Further analysis of the NOV25a protein yielded the following properties shown in Table 25B.

TABLE 25B
Protein Sequence Properties NOV25a

| | |
|---|---|
| PSort | 0.4600 probability located in plasma membrane; |
| analysis: | 0.1226 probability located in microbody (peroxisome); 0.1000 |
| | probability located in endoplasmic reticulum |
| | (membrane); 0.1000 probability located in endoplasmic |
| | reticulum (lumen) |
| SignalP | Cleavage site between residues 25 and 26 |
| analysis: | |

A search of the NOV25a protein against the Geneseq database, a proprietary database that contains sequences published in patents and patent publication, yielded several homologous proteins shown in Table 25C.

TABLE 25C
Geneseq Results for NOV25a

| | | NOV25a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| AAW82791 | Human RON receptor protein - Homo | 7 . . . 1377 | 483/1424 | (33%) | 0.0 |
| | sapiens, 1400 aa. [WO9855141-A1, | 7 . . . 1363 | 710/1424 | (48%) | |
| | 10-DEC-1998] | | | | |
| AAW94409 | Human Met proto-oncogene protein | 501 . . . 817 | 300/317 | (94%) | e−175 |
| | 501-850 - Homo sapiens, 300 aa. | 1 . . . 300 | 300/317 | (94%) | |
| | [US5871959-A, 16-FEB-1999] | | | | |
| AAY43976 | Human protein kinase #25 - Homo | 1094 . . . 1362 | 264/269 | (98%) | e−154 |
| | sapiens, 266 aa. [US5958784-A, | 1 . . . 266 | 265/269 | (98%) | |
| | 28-SEP-1999] | | | | |
| AAW94410 | Mouse Met proto-oncogene protein | 501 . . . 817 | 256/317 | (80%) | e−152 |
| | 501-850 - Mus sp, 299 aa. | 1 . . . 299 | 279/317 | (87%) | |
| | [US5871959-A, 16-FEB-1999] | | | | |
| AAR50089 | Sequence of human Met between the | 501 . . . 817 | 254/317 | (80%) | e−143 |
| | Ndel - PvuII sites, residues 501-850 - | 1 . . . 300 | 255/317 | (80%) | |
| | Homo sapiens, 300 aa. [WO9406909- | | | | |
| | A, 31-MAR-1994] | | | | |

In a BLAST search of public sequence databases, the NOV25a protein was found to have homology to the proteins shown in the BLASTP data in Table 25D.

TABLE 25D
Public BLASTP Results for NOV25a

| | | NOV25a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P08581 | Hepatocyte growth factor receptor | 1 . . . 1408 | 1389/1408 (98%) | 0.0 |
| | precursor (EC 2.7.1.112) (Met proto- | 1 . . . 1390 | 1390/1408 (98%) | |
| | oncogene tyrosine kinase) (c-met) (HGF | | | |
| | receptor) (HGF-SF receptor) - Homo | | | |
| | sapiens (Human), 1390 aa. | | | |
| P16056 | Hepatocyte growth factor receptor | 1 . . . 1399 | 1237/1399 (88%) | 0.0 |
| | precursor (EC 2.7.1.112) (Met proto- | 1 . . . 1379 | 1315/1399 (93%) | |
| | oncogene tyrosine kinase) (c-met) (HGF | | | |
| | receptor) (HGF-SF receptor) - Mus | | | |
| | musculus (Mouse), 1379 aa. | | | |
| P97523 | Hepatocyte growth factor receptor | 1 . . . 1399 | 1220/1401 (87%) | 0.0 |
| | precursor (EC 2.7.1.112) (Met proto- | 1 . . . 1382 | 1302/1401 (92%) | |
| | oncogene tyrosine kinase) (c-met) (HGF | | | |
| | receptor) (HGF-SF receptor) - Rattus | | | |
| | norvegicus (Rat), 1382 aa. | | | |
| Q90975 | TYROSINE KINASE - Gallus gallus | 1 . . . 1399 | 1010/1401 (72%) | 0.0 |
| | (Chicken), 1382 aa. | 1 . . . 1382 | 1172/1401 (83%) | |
| Q9W650 | C-MET/HEPATOCYTE GROWTH | 6 . . . 1399 | 869/1397 (62%) | 0.0 |
| | FACTOR RECEPTOR - Xenopus laevis | 2 . . . 1375 | 1083/1397 (77%) | |
| | (African clawed frog), 1375 aa. | | | |

PFam analysis indicates that the NOV25a protein contains the domains shown in the Table 25E.

TABLE 25E
Domain Analysis of NOV25a

| | | Identities/ | | |
|---|---|---|---|---|
| | | Similarities | | |
| | NOV25a Match | for the Matched | | Expect |
| Pfam Domain/ | Region | Region | | Value |
| Sema: domain 1 of 1 | 55 . . . 500 | 117/493 | (24%) | 5e−172 |
| | | 419/493 | (85%) | |
| integrin_B: | 525 . . . 543 | 7/19 | (37%) | 0.39 |
| domain 1 of 1 | | 14/19 | (74%) | |
| Plexin_repeat: | 519 . . . 562 | 22/67 | (33%) | 1.6e−14 |
| domain 1 of 1 | | 41/67 | (61%) | |
| TIG: domain 1 of 3 | 563 . . . 655 | 28/106 | (26%) | 2.7e−21 |
| | | 80/106 | (75%) | |
| TIG: domain 2 of 3 | 657 . . . 739 | 31/104 | (30%) | 8e−21 |
| | | 71/104 | (68%) | |
| TIG: domain 3 of 3 | 762 . . . 854 | 28/113 | (25%) | 5.8e−07 |
| | | 67/113 | (59%) | |
| pkinase: domain 1 of 1 | 1096 . . . 1355 | 85/297 | (29%) | 2.1e−91 |
| | | 218/297 | (73%) | |

### Example 26

The NOV26 clone was analyzed, and the nucleotide and polypeptide sequences are shown in Table 26A.

TABLE 26A
NOV26 Sequence Analysis

| | |
|---|---|
| SEQ ID NO: 119 | 2317 bp |

| | |
|---|---|
| NOV26a, | AAAACTCCAGGACGCGGAGGAGGCTAGTGGCAGTACCTGGGCACCCTGACCCTCCCCA |
| CG93443-01 DNA | CAGGCCAGAGCCCACCCTCCTGCTCATGAGGGCAGACAGGCCTTTCCAGGGACACAGT |
| Sequence | CCCTCTTCTCCCCAGGACCCCAGGGCCAACTCCCCCTGCCGGCCCTCTGCCATCAAAT |
| | TGGCAGTGGCTCCAGGGGAGTCCCCTGGGGATGGGGCACCACTGTTGGGGACCCCTCT |
| | GCGTGCACCCCTGTAGTTGGGGAAGCAGGACAGGGGCCTGGGGAGACGGAAGGGCGCC |
| | AGGGGTTGAGAGAGGATGGTGGACGTTGTTGGACTTCAAAGGGAAACAGGCCCTCGGG |
| | GAAGCCCCTGGCCAGGCCTGCCTCTCCCCTCCCTGGTGGGCCCAGCGCCCCTGCTCAC |
| | TTGTCTCTGCCCACAGTGCCTGTCTGTGGAGGACGCCCTGGGCCTGGGCGAGCCTGAG |
| | GGGTCAGGGCTGCCCCCGGCCCCGGTCCTGGAGGCCAGGTACGTCGCCCCCCTCAGTG |
| | CCGCCGCCGTCCTGTACCTCAGCAACCCCGAGGGCACCTGTGAGGACGCTCGGGCTGG |
| | CCTCTCGGCCTCTCATGCAGACCACCTCCTGGCCCTGCTCGAGAGCCCCAAGGCCCTG |
| | ACCCCGGGCCTGAGCTGGCTGCTGCAGAGGATGCAGGCCCGGGCTGCCGGCCAGACCC |
| | CCAAGACGGCCTGCGTAGATATCCCTCAGCTGCTGGAGGAGCCGGTGGGGGCGGGGGC |
| | TCCGGGCAGTGCTGGCGGCGTCCTGGCTGCCCTGCTGGACCATGTCAGGAGCGGGTCT |
| | TGCTTCCACGCCTTGCCGAGCCCTCAGTACTTCGTGGACTTTGTGTTCCAGCAGCACA |
| | GCAGCGAGGTCCCTATGACGCTGGCCGAGCTGTCAGCCTTGATGCAGCGCCTGGGGGT |
| | GGGCACCGAGGCCCACAGTGACCACAGTCATCGGCACAGGGGAGCCAGCAGCCGGGAC |
| | CCTGTGCCCCTCATCAGCTCCAGCAACAGCTCCAGTGTGTGGGACACGGTATGCCTGA |
| | GTGCCAGGGACGTGATGGCTGCATATGGACTGTCGGAACAGGCTCGGGTGACCCCGGA |
| | GGCCTGGGCCCAACTGAGCCCTGCCCTGCTCCAACAGCAGCTGAGTGGAGCCTACACC |
| | TCCCAGTCCAGGCCCCCCGTCCAGGACCAGCTCAGCCAGTCACAGAGATATCTGTACG |
| | GCTCCCTGGCCACGCTGCTCATCTGCCTCTGCGCGGTCTTTGGCCTCCTGCTGCTGAC |
| | CTGCACTGGCTGCAGGGGGGTCGCCCACTACATCCTGCAGACCTTCCTGAGCCTGGCA |
| | GTGGGTGCACTCACTGGGGACGCTGTCCTGCATCTGACGCCCAAGGTGCTGGGGCTGC |
| | ATACACACAGCGAACAGGGCCTCAGCCCACAGCCCACCTGGCGCCTCCTGGCTATGCT |
| | GGCCGGGCTCTACGCCTTCTTCCTGTTTGAGAACCTCTTCAATCTCCTCCTGCCCAGG |
| | GACCCGGAGGACCTGGAGGACGCGCCCTGCGGCCACAGCAGCCATAGCCACGGCGGCC |
| | ACAGCCACGGTGTGTCCCTGCAGCTGGCACCCAGCGAGCTCCGGCAGCCCAAGCCCCC |
| | CCACGAGGGCTCCCCCCCAGACCTGGTGGCGGAGGAGAGCCCGGAGCTGCTGAACCCT |
| | GAGCCCAGGAGACTCAGCCCAGAGTTGAGGCTACTGCCCTATATGATCACTCTGGGCG |
| | ACGCCGTGCACAACTTCGCCGACGGGCTGGCCGTGGGCGCCGCCTTCGCGTCCTCCTG |
| | GAAGACCGGGCTGGCCACCTCGCTGGCCGTGTTCTGCCACGAGTTGCCACACGAGCTG |
| | GGGGACTTCGCCGCCTTGCTGCACGCGGGGCTGTCCGTGCGCCAAGCACTGCTGCTGA |
| | ACCTGGCCTCCGCGCTCACGGCCTTCGCTGGTCTTACGTGGCACTCGCGGTTGGAGTC |
| | AGCGAGGAGAGCGAGGCCTGGATCCTGGCAGTGGCCACCGGCCTGTTCCTTACGTAGC |
| | ACTCTGCGACATGCTCCCGGCGATGTTGAAAGTACGGGACCCGCGGCCCCTGGCTCCT |
| | CTTCCTGCTGCACAACGTGGGCCTGCTGGGCGGCTGGACCGTCCTGCTGCTGCTGTCC |
| | CTGTACGAGGATGACATCACCTTCTGATACCCTGCCCTAGTCCCCCACCTTTGACTTA |
| | AGATCCCACACCTCACAAACCTACAGCCCAGAAACCCAGAAGCCCCTATAGAGCCCCC |
| | AGTCCCAACTCCAGTAAAGACACTCTTGTCCCTTGGAAAAAAAAAAAAAAAAAAA |

| ORF Start: ATG at 306 | ORF Stop: TAG at 2184 | |
|---|---|---|
| SEQ ID NO: 120 | 626 aa | MW at 66248.5 kD |

| | |
|---|---|
| NOV26a, | MVDVVGLERETGPRGSPWPGLPLPSLVGPAPLLTCLCPQCLSVEDALGLGEPEGSGLP |
| CG93443-01 Protein | PGPVLEARYVARLSAAAVLYLSNPEGTCEDARAGLWASHADHLLALLESPKALTPGLS |
| Sequence | WLLQRMQARAAGQTPKTACVDIPQLLEEAVGAGAPGSAGGVLAALLDHVRSGSCFHAL |
| | PSPQYFVDFVFQQHSSEVPMTLAELSALMQRLGVGREAHSDHSHRHRGASSRDPVPLI |
| | SSSNSSSVWDTVCLSARDVMAAYGLSEQAGVTPEAWAQLSPALLQQQLSGAYTSQSRP |
| | PVQDQLSQSERYLYGSLATLLICLCAVFGLLLLTCTGCRGVAHYILQTFLSLAVGALT |
| | GDAVLHLTPKVLGLHTHSEEGLSPQPTWRLLAMLAGLYAFFLFENLFNLLLPRDPEDL |
| | EDGPCGHSSHSHGGHSHGVSLQLAPSELRQPKPPHEGSRADLVAEESPELLNPEPRRL |
| | SPELRLLPYMITLGDAVHNFADGLAVGAAFASSWKTGLATSLAVFCHELPHELGDFAA |
| | LLHAGLSVRQALLLNLASALTAFAGLTWHSRLESARRARPGSWQWPPACSLRSTLRHA |
| | PGDVESTGPAAPGSSSCCTTWACWAAGPSCCCCPCTRMTSPSDTLP |

Further analysis of the NOV26a protein yielded the following properties shown in Table 26B.

TABLE 26B
Protein Sequence Properties NOV26a

| | |
|---|---|
| PSort | 0.7000 probability located in plasma membrane; |
| analysis: | 0.3048 probability located in microbody (peroxisome); 0.2000 |
| | probability located in endoplasmic reticulum |
| | (membrane); 0.1000 probability located in mitochondrial |
| | inner membrane |
| SignalP | Cleavage site between residues 43 and 44 |
| analysis: | |

A search of the NOV26a protein against the Geneseq database, a proprietary database that contains sequences published in patents and patent publication, yielded several homologous proteins shown in Table 26C.

TABLE 26C
Geneseq Results for NOV26a

| | | NOV26a | Identities/ | |
|---|---|---|---|---|
| Geneseq | Protein/Organism/Length | Residues/ | Similarities for | Expect |
| Identifier | [Patent #, Date] | Residues | Region | Value |
| AAE06574 | Human protein having hydrophobic | 37 . . . 548 | 508/512 (99%) | 0.0 |
| | domain, HP10755 - Homo sapiens, | 62 . . . 573 | 509/512 (99%) | |
| | 647 aa. [WO200149728-A2, | | | |
| | 12-JUL-2001] | | | |
| AAE01677 | Human gene 6 encoded secreted protein | 37 . . . 548 | 508/512 (99%) | 0.0 |
| | HWLHM66, SEQ ID NO: 89 - Homo | 62 . . . 573 | 509/512 (99%) | |
| | sapiens, 647 aa. [WO200134767-A2, | | | |
| | 17-MAY-2001] | | | |
| AAB74710 | Human membrane associated protein | 37 . . . 548 | 507/512 (99%) | 0.0 |
| | MEMAP-16 - Homo sapiens, 647 aa. | 62 . . . 573 | 508/512 (99%) | |
| | [WO200112662-A2, 22-FEB-2001] | | | |
| AAE01709 | Human gene 6 encoded secreted protein | 37 . . . 410 | 365/374 (97%) | 0.0 |
| | HWLHM66, SEQ ID NO: 121 - Homo | 62 . . . 435 | 366/374 (97%) | |
| | sapiens, 440 aa. [WO200134767-A2, | | | |
| | 17-MAY-2001] | | | |
| AAB59035 | Breast and ovarian cancer associated | 436 . . . 548 | 107/113 (94%) | 2e−54 |
| | antigen protein sequence SEQ ID 743 - | 18 . . . 130 | 108/113 (94%) | |
| | Homo sapiens, 204 aa. [WO200055173- | | | |
| | A1, 21-SEP-2000] | | | |

In a BLAST search of public sequence databases, the NOV26a protein was found to have homology to the proteins shown in the BLASTP data in Table 26D.

TABLE 26D
Public BLASTP Results for NOV26a

| | | NOV26a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NXC4 | CDNA FLJ20327 FIS, CLONE | 1 . . . 626 | 626/626 (100%) | 0.0 |
| | HEP10012 - Homo sapiens (Human), | 1 . . . 626 | 626/626 (100%) | |
| | 626 aa. | | | |
| Q9H6T8 | CDNA: FLJ21884 FIS, CLONE | 37 . . . 548 | 508/512 (99%) | 0.0 |
| | HEP02863 - Homo sapiens (Human), | 62 . . . 573 | 509/512 (99%) | |
| | 647 aa. | | | |
| Q9DAT9 | 1600025H15RIK PROTEIN - Mus | 37 . . . 548 | 365/531 (68%) | 0.0 |
| | musculus (Mouse), 660 aa. | 61 . . . 586 | 415/531 (77%) | |
| Q95KA5 | HYPOTHETICAL 72.8 KDA PROTEIN - | 130 . . . 548 | 141/444 (31%) | 2e−51 |
| | Macaca fascicularis (Crab eating | 177 . . . 580 | 217/444 (48%) | |
| | macaque) (Cynomolgus monkey), 654 aa. | | | |
| Q96NN4 | CDNA FLJ30499 FIS, CLONE | 174 . . . 548 | 127/393 (32%) | 7e−49 |
| | BRAWH2000443, WEAKLY SIMILAR | 227 . . . 580 | 197/393 (49%) | |
| | TO HUMAN BREAST CANCER, | | | |
| | ESTROGEN REGULATED LIV-1 | | | |
| | PROTEIN (LIV-1) MRNA - Homo | | | |
| | sapiens (Human), 654 aa. | | | |

PFam analysis indicates that the NOV26a protein contains the domains shown in the Table 26E.

TABLE 26E
Domain Analysis of NOV26a

| | NOV26a | Identities/ | |
|---|---|---|---|
| | Match | Similarities | Expect |
| Pfam Domain | Region | for the Matched Region | Value |
| Zip: domain 1 of 1 | 467 . . . 618 | 37/184 (20%) | 0.00025 |
| | | 108/184 (59%) | |

### Example 27

The NOV27 clone was analyzed, and the nucleotide and polypeptide sequences are shown in Table 27A.

TABLE 27A
NOV27 Sequence Analysis

| | |
|---|---|
| SEQ ID NO: 121 | 2852 bp |

| | |
|---|---|
| NOV27a, | GTGTGCAGTAAACTGGAATGCTCTCCCTCGCTCACTCCTCAGTGTAGCAGTGATCTGA |
| CG50838-01 DNA | AGCAGGACAAGCTCAGCCTGCAGCTGCCGTGGGCTTTGTGTGGACTGGACGCAGAGCT |
| Sequence | TGGGAGACGGGGGAGGGCTATTACTCCAATTCACTGTCAATGGAATTACAGCTATAGC |
| | GGCAGTGTATATAGGATTGCTTTTTCTCGTCTTCCTGGAGATGCTCAGTCCCAGTATA |
| | TTTTAAGGAAGAGAAATATAAAGGAAATTTAGTATGCCTCCTTTTCTTTAAATGAAGA |
| | ATTTAGTTTCCTTTACTTCTTAAAAGAGAATACCTGTTCTTGTATAACGTGACTGCAC |
| | CAGACATTCTGAAAAATCAGCAAGAAGCAAAAGCTGGAAATAGCTATTTCACAGCAGG |
| | GTTCTGAAGTAACGGAAGCTACCTTGTATAAAGACCTCAACACTGCTGACCATGATCA |
| | GCGCAGCCTGGAGCATCTTCCTCATCGGGACTAAAATTGGGCTGTTCCTTCAAGTAGC |
| | ACCTCTATCAGTTATGGCTAAATCCTGTCCATCTGTGTGTCGCTGCGATGCGGGTTTC |
| | ATTTACTGTAATGATCGCTTTCTGACATCCATTCCAACAGGAATACCAGAGGATGCTA |
| | CAACTCTCTACCTTCAGAACAACCAAATAAATAATGCTGGGATTCCTTCAGATTTGAA |
| | AAACTTGCTGAAAGTAGAAAGAATATACCTATACCACAACAGTTTACATGAATTTCCT |
| | ACCAACCTCCCAAAGTATGTAAAAGAGTTACATTTGCAAGAAAATAACATAAGGACTA |
| | TCACTTATGATTCACTTTCAAAAATTCCCTATCTGGAAGAATTACATTTAGATGACAA |
| | CTCTGTCTCTGCAGTTAGCATAGAAGAGGGAGCATTCCCAGACAGCAACTATCTCCGA |
| | CTGCTTTTCCTGTCCCGTAATCACCTTAGCACAATTCCCTGGGGTTTCCCCAGGACTA |
| | TAGAACAACTACGCTTGGATGATAATCGCATATCCACTATTTCATCACCATCTCTTCA |
| | AGGTCTCACTAGTCTAAAACGCCTGGTTCTAGATGGAAACCTGTTGAACAATCATGGT |
| | TTAGGTGACAAAGTTTTCTTCAACCTAGTTAATTTGACAGAGCTGTCCCTGGTGCGGA |
| | ATTCCCTGACTGCTGCACCAGTAAACCTTCCACGCACAAACCTGAGGAAGCTTTATCT |
| | TCAAGATAACCACATCAATCGGGTGCCCCCAAATGCTTTTTCTTATCTAAGGCAGCTC |
| | TATCGACTGGATATGTCCAATAATAACCTAAGTAATTTACCTCAGGGTATCTTTGATG |
| | ATTTGGACAATATAACACAACTGATTCTTCGCAACAATCCCTGGTATTGCGGGTGCAA |
| | GATGAAATGCGTACGTGACTGGTTACAATCACTACCTGTGAAGGTCAACGTGCGTGGG |
| | CTCATGTGCCAAGCCCCAGAAAAGGTTCGTGGGATGGCTATTAAGGATCTCAATGCAG |
| | AACTGTTTGATTGTAAGGACAGTGGGATTGTAAGCACCATTCAGATAACCACTGCAAT |
| | ACCCAACACAGTCTATCCTGCCCAAGGACAGTGGCCAGCTCCAGTGACCAAACAGCCA |
| | GATATTAAGAACCCCAAGCTCACTAAGGATCAACAAACCACAGGGAGTCCCTCAAGAA |
| | AAACAATTACAATTACTGTGAAGTCTGTCACCTCTGATACCATTCATATCTCTTGGAA |
| | ACTTGCTCTACCTATGACTGCTTTGAGACTCAGCTGGCTTAAACTGGGCCATAGCCCG |
| | GCATTTGGATCTATAACAGAAACAATTGTAACAGGCGAACGCAGTGAGTACTTGGTCA |
| | CAGCCCTGGAGCCTGATTCACCCTATAAAGTATGCATGGTTCCCATGGAAACCAGCAA |
| | CCTCTACCTATTTGATGAAACTCCTGTTTGTATTGAGACTGAAACTGCACCCCTTCGA |
| | ATGTACAACCCTACAACCACCCTCAATCGAGAGCAAGAGAAAGAACCTTACAAAAACC |
| | CCAATTTACCTTTGGCTGCCATCATTGGTGGGGCTGTGGCCCTGGTTACCATTGCCCT |
| | TCTTGCTTTAGTGTGTTGGTATGTTCATAGGAATGGATCGCTCTTCTCAAGGAACTGT |
| | GCATATAGCAAAGGGAGGAGAAGAAAGGATGACTATGCAGPAGCTGGCACTAAGAAGG |
| | ACAACTCTATCCTGGAAATCAGGGAAACTTCTTTTCAGATGTTACCPATAAGCAATGA |
| | ACCCATCTCGAAGGAGGAGTTTGTAATACACACCATATTTCCTCCTAATGGAATGAAT |
| | CTGTACAAAAACAATCACAGTGAAAGCAGTAGTAACCGAAGCTACAGAGACAGTGGTA |
| | TTCCAGACTCAGATCACTCACACTCATGATGCTGAAGGACTCACAGCAGACTTGTGTT |
| | TTGGGTTTTTTAAACCTAAGGGAGGTGATGGTAGGAACCCTGTTCTACTGCAAAACAC |
| | TGGAAAAAGAGACTGAAAAAAAGCAATGTACTGTACATTTGCCATATAATTTATATTT |
| | AAGAACTTTTTATTAAAAGTTTCAAATTTCAGGTTACTGCTGCGATTGATGTAGTGGA |
| | CATGCCTGAACACAATTCTATATTTTAGTATTTTTTAGTAATTTGTACTGTATTTTCC |
| | TTGCAAATATTGGAGTTATAAACCATTTACTTTGTGTTCTACTGAGTAAGATGACTTG |
| | TTGACTGTGAAAGTGAATTTTCTTGCTGTGTCGAACAATCAGGACTGCATTCATATGA |
| | GATCCTTGTAGTATAAGCACAGGCCATTTTTCACTTTGGTATTAATAAAATGTAAAAA |
| | AAAAATTGGT |

| ORF Start: ATG at 458 | ORF Stop: TGA at 2405 | |
|---|---|---|
| SEQ ID NO: 122 | 649 aa | MW at 72993.5 kD |

| | |
|---|---|
| NOV27a, | MISAAWSIFLIGTKIGLFLQVAPLSVMAKSCPSVCRCDAGFIYCNDRFLTSIPTGIPE |
| CG50838-01 Protein | DATTLYLQNNQINNAGIPSDLKNLLKVERIYLYHNSLDEFPTNLPKYVKELHLQENNI |
| Sequence | RTITYDSLSKIPYLEELHLDDNSVSAVSIEEGAFRDSNYLRLLFLSRNHLSTIPWGLP |
| | RTIEELRLDDNRISTTSSPSLQGLTSLKRLVLDGNLLNNHGLGDKVFFNLVNLTELSL |
| | VRNSLTAAPVNLPGTNLRKLYLQDNHINRVPPNAFSYLRQLYRLDMSNNNLSNLPQGI |
| | FDDLDNITQLILRNNPWYCGCKMKWVRDWLQSLPVKVNVRGLMCQAPEKVRGMAIKDL |
| | NAELFDCKDSGIVSTIQITTAIPNTVYPAQGQWPAPVTKQPDIKNPKLTKDQQTTGSP |
| | SRKTITITVKSVTSDTIHISWKLALPMTALRLSWLKLGHSPAFGSITETIVTGERSEY |
| | LVTALEPDSPYKVCMVPMETSNLYLFDETPVCIETETAPLRMYNPTTTLNREQEKEPY |
| | KNPNLPLAAIICGAVALVTIALLALVCWYVHRNGSLFSRNCAYSKGRRRKDDYAEAGT |
| | KKDNSILEIRETSFQMLPISNEPISKEEFVIHTIFPPNGMNLYKNNHSESSSNRSYRD |
| | SGIPDSDHSHS |

Further analysis of the NOV27a protein yielded the following properties shown in Table 27B.

TABLE 27B
Protein Sequence Properties NOV27a

| | |
|---|---|
| PSort | 0.6976 probability located in plasma membrane; 0.6400 |
| analysis: | probability located in endoplasmic reticulum (membrane); |
| | 0.1900 probability located in Golgi body; |
| | 0.1000 probability located in endoplasmic reticulum (lumen) |
| SignalP | Cleavage site between residues 29 and 30 |
| analysis: | |

A search of the NOV27a protein against the Geneseq database, a proprietary database that contains sequences published in patents and patent publication, yielded several homologous proteins shown in Table 27C.

TABLE 27C
Geneseq Results for NOV27a

| | | NOV27a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAU29215 | Human PRO polypeptide sequence | 1 . . . 649 | 649/649 (100%) | 0.0 |
| | #192 - Homo sapiens, 649 aa. | 1 . . . 649 | 649/649 (100%) | |
| | [WO200168848-A2, 20-SEP-2001] | | | |
| AAB87591 | Human PRO1865 - Homo sapiens, | 1 . . . 649 | 649/649 (100%) | 0.0 |
| | 649 aa. [WO200116318-A2, | 1 . . . 649 | 649/649 (100%) | |
| | 08-MAR-2001] | | | |
| AAB70533 | Human PRO3 protein sequence SEQ | 1 . . . 649 | 649/649 (100%) | 0.0 |
| | ID NO: 6 - Homo sapiens, 649 aa. | 1 . . . 649 | 649/649 (100%) | |
| | [WO200110902-A2, 15-FEB-2001] | | | |
| AAB93758 | Human protein sequence SEQ ID | 1 . . . 649 | 648/649 (99%) | 0.0 |
| | NO: 13435 - Homo sapiens, 649 aa. | 1 . . . 649 | 649/649 (99%) | |
| | [EP1074617-A2, 07-FEB-2001] | | | |
| AAM93408 | Human polypeptide, SEQ ID NO: | 1 . . . 649 | 648/649 (99%) | 0.0 |
| | 3016 - Homo sapiens, 649 aa. | 1 . . . 649 | 648/649 (99%) | |
| | [EP1130094-A2, 05-SEP-2001] | | | |

In a BLAST search of public sequence databases, the NOV27a protein was found to have homology to the proteins shown in the BLASTP data in Table 27D.

TABLE 27D
Public BLASTP Results for NOV27a

| | | NOV27a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAC33411 | SEQUENCE 5 FROM PATENT | 1 . . . 649 | 649/649 (100%) | 0.0 |
| | WO0110902 - Homo sapiens (Human), | 1 . . . 649 | 649/649 (100%) | |
| | 649 aa. | | | |
| Q96KB1 | CDNA FLJ14391 FIS, CLONE | 1 . . . 649 | 648/649 (99%) | 0.0 |
| | HEMBA1003077, WEAKLY | 1 . . . 649 | 649/649 (99%) | |
| | SIMILAR TO SLIT PROTEIN | | | |
| | (Human), 649 aa. | | | |
| Q9P259 | KIAA1469 PROTEIN - Homo sapiens | 1 . . . 649 | 648/649 (99%) | 0.0 |
| | (Human), 662 aa (fragment). | 14 . . . 662 | 648/649 (99%) | |
| Q9NZU0 | LEUCINE-RICH REPEAT | 1 . . . 649 | 648/649 (99%) | 0.0 |
| | TRANSMEMBRANE PROTEIN | 1 . . . 649 | 648/649 (99%) | |
| | FLRT3 (DJ581I13.1) - Homo sapiens | | | |
| | (Human), 649 aa. | | | |
| Q96K39 | CDNA FLJ14788 FIS, CLONE | 27 . . . 649 | 622/623 (99%) | 0.0 |
| | NT2RP4000925, WEAKLY SIMILAR | 1 . . . 623 | 622/623 (99%) | |
| | TO FIBROMODULIN PRECURSOR - | | | |
| | Homo sapiens (Human), 623 aa. | | | |

PFam analysis indicates that the NOV27a protein contains the domains shown in the Table 27E.

TABLE 27E
Domain Analysis of NOV27a

| | | Identities/ | |
|---|---|---|---|
| | | Similarities for | |
| | NOV27a | the | Expect |
| Pfam Domain | Match Region | Matched Region | Value |
| LRRNT: domain 1 of 1 | 30 . . . 57 | 10/31 (32%) | 0.00034 |
| | | 19/31 (61%) | |
| LRR: domain 1 of 10 | 59 . . . 83 | 7/26 (27%) | 44 |
| | | 20/26 (77%) | |
| LRR: domain 2 of 10 | 84 . . . 103 | 8/25 (32%) | 75 |
| | | 16/25 (64%) | |
| LRR: domain 3 of 10 | 105 . . . 128 | 7/25 (28%) | 12 |
| | | 18/25 (72%) | |
| LRR: domain 4 of 10 | 129 . . . 154 | 9/26 (35%) | 76 |
| | | 20/26 (77%) | |
| LRR: domain 5 of 10 | 155 . . . 174 | 10/25 (40%) | 5.4 |
| | | 16/25 (64%) | |
| LRR: domain 6 of 10 | 176 . . . 199 | 8/25 (32%) | 0.1 |
| | | 20/25 (80%) | |
| LRR: domain 7 of 10 | 200 . . . 225 | 9/26 (35%) | 62 |
| | | 18/26 (69%) | |
| LRR: domain 8 of 10 | 226 . . . 245 | 11/25 (44%) | 63 |
| | | 16/25 (64%) | |
| LRR: domain 9 of 10 | 248 . . . 271 | 9/25 (36%) | 0.00033 |
| | | 20/25 (80%) | |
| LRR: domain 10 of 10 | 272 . . . 295 | 13/25 (52%) | 0.011 |
| | | 19/25 (76%) | |
| LRRCT: domain 1 of 1 | 305 . . . 356 | 17/54 (31%) | 9.4e−13 |
| | | 42/54 (78%) | |
| fn3: domain 1 of 1 | 405 . . . 485 | 18/90 (20%) | 0.22 |
| | | 54/90 (60%) | |

### Example 28

The NOV28 clone was analyzed, and the nucleotide and polypeptide sequences are shown in Table 28A.

TABLE 28A
NOV28 Sequence Analysis

| | |
|---|---|
| SEQ ID NO: 123 | 3373 bp |

| | |
|---|---|
| NOV28a, | TTGGATTGTCCAAGCCACAGATAAAGGGATGCCCAGGCTTTCTAATACGACTGTAATCA |
| CG58567-01 DNA | AGGTACAGGTGACTGATATAAATGACAATGCCCCAGCTTTTCTCCCCTCTGAAGCAGT |
| Sequence | GGAAATTACAGAAGTCATGACTATTTCAGAAGATTCTTTGCCTGGTGTAATTGTGACT |
| | CATGTGTCAGTTCATGATGTGGATTTGAATTCAGCTTTCATATTCAGTTTTGCCAAAG |
| | AGAGTAATCCTGGAACCAAGTTTGCTATTGATCACAACACTGGAGTGGTGGTGTTCGT |
| | GAAAACATTGGATTTTGAAGAAATGACTGAATATGAGCTGCTCATCCAAATTTCTGAT |
| | TCAGTGCACTACACAGAGGGAGCACTTGTAGTCCGTGTGCTGGATGTCPATGATAATC |
| | CACCAGTGTTTTCTCAACATTTCTATCAGCTCACAGTTCCTGAATCAATACCTGTGGG |
| | GTATTCAGTGCTGACTCTGTCAGCCACAGACTTAGAAAGCAATGAGAACATTTCTTAC |
| | AGAATTCTATCCTCTTCTAAGGAATTCTCCATTGATCCTAAGAATGGCACAATATTTA |
| | CTATCAGTCCCGTATTACTTCTGGATACAATATCAACAACTCGATTTCTTGTGGAAGC |
| | CAGTGATGGTGGAAATCCTGACCCGAGAGCTCTTACTTTAGTGGAGATAGGAATAGAA |
| | GATATGAACAATTATGCCCCTGAATTCACAGTCAAATCCTATAATCTTAGCCTAAGTG |
| | AGGATGCTCTGGTTGGAAGCACGCTTGTTACATTTTCAAACATCGACCATGACTGGAC |
| | CCGTGAAAACACATATGTTGAATATTCCATCATCAGTGGTAATTCACAGAACAATTTT |
| | CATGTGGAAACTAAGTTCTTTCATTCAGAATATCCTTATAAGCAAGTCGGTTATCTTG |
| | TGTTGCTTCACAGTCTGGACAGAGAAGCAAGTGCTAGCCATGAGCTTGTCATTCTGGC |
| | ATCTGACAGTGGCTGCCCTCCATTGAGTTCCACAGCTGTCATATCAATACAAGTACTT |
| | GATGTCAATGACAATCCCCCAAACTTCAGCAGCCTGAGCTATCACACCCATGTCAAGG |
| | AAAGCACCCCTCTAGGGAGTCACATCACTGTGGTCTCAGCAAATGACCGTGACACAGG |
| | GTCACATGCAGAAATCATCTACAACATCATCTCTGGAAATGAGAAGGGACATTTTTAC |
| | TTAGAAGAAAACACTGGAGTTCTTTATTTGATTAAACCTCTGGATTATGAAAAAATGA |
| | CAAAATTCACCTTAACTGTCCAAGCTTCAGATGCAGAAAAGAAACATTTTTCTTTTGC |
| | AGTTGTGTTTGTCAGTGTCCTCGATGATAACGACCATGCACCTCAGTTTATCTTCTCA |
| | AGCTTCAGCTGTATTGTTCCAGAAAATCTGCCTATTTCCTCTACCATATGCTCTATAA |
| | ATGCTCTGGATTTTGATGCTGGTCCGTATGGAGAATTGACCTATTCTATTGTATCACC |
| | CTGTTTTCTCACTCATGGAATGTCTTATGATCATGATCTCTTCCTCATTGACCCTTTG |
| | ACAGGCGATATTCATGCTAAGCAAATCCTTGACTATGAAAATGGCAATAAATACTGCC |
| | TCACAGTCCAAGCCAAAGACAAAGGTGATGCAACTGCCTCCTTAGTGGTCTGGGTGGA |
| | TATTGAAGGGATAGATGAATTTGAGCCCATTTTCACTCAAGATCAGTATTTTTTCACC |
| | CTCCCAGAAAAGAATAAAGACAGACAGTTGATTGGCAGAGTGGAAGCCTCAGATGCAG |
| | ATGCTGGTATTGATGGAGTCATTCTTTACTCCCTTGGAACCTCATCTCCTTTCTTTTC |
| | AGTAAATAGAACCAATGGAAATATTTATTTCATTACAGCCCTTCCCCTAATAAAAAGT |
| | CAACTCAACAAAGAAGACACCTTGGAAATGAAAATAATCGCTCATAGTCCCAAATCAG |
| | ATTCCAAGTTTGCATCTTGCACTGTTTTTGTGAATGTGTCTTTCTCCTCTGAAGGAAC |
| | ACCCTTGGCAGTGTTCGCCAGCAGCTTTTCAATCAGCCTGGTGGTCTCCTTTTTAGTG |
| | TTTCTGATACTCATCTGCATTCTAATTGTAATGATTTTAAGACATAAACAAAAAGACA |
| | CAATAAACAATTATGAGGAGAAGAAAACCTCATCTTTAGATGCGGACTTGAGAGTGAC |
| | CCGGGATGCCAGTGTGCTCAAAGCCTTCCAGAAAACTGACGACTGCAGTAACGAGGTG |
| | GTCCCTGTGGATGCCACTCCGGAATCGTTGAGTTTAATAAGTATCATGGAGAAGGATA |
| | TTGTCAATCTGTACAGATACTCAAACTCCAGTGGCCACTGTTCTGTGGAAGGAGAAAC |
| | TGCAGAAGATAAGGAAATCCAGAGGATAAATGAGCATCCCTACAGAAAGTGCTCAGAC |
| | TCAGCTCTGAGTGACCACGAGTCCAGGGTGCCAGACTCGGGTATCCCGAGGGACTCAG |
| | ACCAGCTCTCCTGCCTATCTGGGGAAACTGATGTGATGCTGACTGCCGAAACAGCAGA |
| | AGCCAGCCAAACATTTGGGGAAGGAGATCAACGGGAAGGCTCCAGCACCACCTGTGCT |
| | CAAAATAATGTGTTACCCCAGACAGTTCAGAAGAGAGAGGCAAAAGAGAGCATCCTGG |
| | CTGACGTTAGAAAAGAGTCTGTCTTTATTTCAGGTGATCAGGAAGTAAGGTGTGCAGC |
| | TCTTTCAACTCAGACGACCTCTGATCATGATGGAAAAGACAACTATCACTGGAATTAT |
| | CTTCTTAGTTGGGAGCCCAAATTCCAACCTCTTGCCTCACTATTTAATGATATTGCAA |
| | AACTAAAGGATGAACATTTGCATATGCCTGGCATTCCAAAAGAGAAGAAATCTTTTGT |
| | TTTTCCACCCCCTTTGATAACAGCAGTAGCCCAGCCTGGGATTAAAGCAGTCCCACCA |
| | AGAATGCCGGCAGTAAACCTGGCGCAGGTGCCTCCGAAACACCCACGCTCTCCCATCC |
| | CCTACCATCTTGGTTCTCTGCCAGAAGGCATGACTCCCAATTTTTCTCCATCTCTTTC |
| | CCTATTGACGATGCAGCCTCCTGCCTTGTCTCCACTGTTGAGAGAAGGAGAATTATTA |
| | GGAACACACATCAGTGGTACATGCCATGAACTTAAAGCAGAAGATGAAGTTCAAATAT |
| | GAAACCACTGGGATGCCAAGTACCTGCTCACCATTGGTCATGAATGAATGAACAAAAT |
| | TTTAGTTAGAGTTTTTAAAACTTCCCCATTAAAGTTTCTCCAATTTCAAAAAAAAAAA |
| | AAAAAAAAA |

| ORF Start: TTG at 1 | ORF Stop: TGA at 3190 | |
|---|---|---|
| SEQ ID NO: 124 | 1063 aa | MW at 117200.4 kD |

| | |
|---|---|
| NOV28a, | LIVQATDKGMPRLSNTTVIKVQVTDINDNAPAFLPSEAVEITEVMTISEDSLPGVIVT |
| CG58567-01 Protein | HVSVHDVDLNSAFIFSFAKESNPGTKFAIDQNTGVVVLVKTLDFEEMTEYELLIQISD |
| Sequence | SVHYTEGALVVRVLDVNDNPPVFSQDFYQVTVPESIPVGYSVLTLSATDLESNENISY |
| | RILSSSKEFSIDPKNGTIFTISPVLLLDTISTTRFLVEASDGGNPDPRALTLVEIGIE |
| | DMNNYAPEFTVKSYNLSLSEDALVGSTLVTFSNIDHDWTRENTYVEYSIISGNSQNNF |
| | HVETKFFHSEYPYKQVGYLVLLHSLDREASASHELVILASDSGCPPLSSTAVISIQVL |
| | DVNDNPPNFSSLSYHTHVKESTPLGSHITVVSANDRDTGSHAEIIYNIISGNEKGHFY |
| | LEENTGVLYLIKPLDYEKNTKFTLTVQASDAEKKHFSFAVVFVSVLDDNDHAPQFMFS |
| | SFSCIVPENLPISSTICSINALDFDAGPYGELTYSIVSPCFLTHGMSYDHDLFLIDPL |
| | TGDIHAKQILDYENGNKYCLTVQAKDKGDATASLVVWVDIEGIDEFEPIFTQDQYFFT |
| | LPEKNKDRQLIGRVEASDADAGIDGVILYSLGTSSPFFSVNRTNGNIYLIRALPLIKS |
| | QLNKEDTLEMKIIAHSPKSDSKFASCTVFVNVSFSSEGTPLAVFASSFSISLVVSFLV |
| | FLILICILIVMILRHKQKDTINNYEEKKTSSLDADLRVTRDASVLKAFQKTDDCSNEV |
| | VPVDATPEWLSLISIMEKDIVNLYRYSNSSGHCSVEGETAEDKEIQRINEHPYRKCSD |
| | SALSDHESRVPDSGIPRDSDQLSCLSGETDVMVTAETAEASQTFGEGDQGEGCSTTCA |
| | QNNVLPQTVQKREAKESILADVRKESVFISGDQEVRCAALSTQTTSDHDGKDNYHWNY |
| | LLSWEPKFQPLASVFNDIAKLKDEHLHMPGIPKEKKSFVFPPPLITAVAQPGIKAVPP |
| | RMPAVNLGQVPPKHPRSPIPYHLGSLPECMTPNFSPSLSLLTMQPPALSPLLREGELL |
| | GTHISGTCHELKAEDEVQI |

| | |
|---|---|
| SEQ ID NO: 125 | 9193 bp |

| | |
|---|---|
| NOV28b, | ATGGAGAAATGTGGGCTTAAAGAGGAAGGCAGTTACGCGGACATAGATCCAGTTGCCC |
| CG58567-05 DNA | ACGATCCGGACGCCGGACTGTTCAGCACTCAGGGCTACACCCTGGTGCAACCGTCCGA |
| Sequence | CCTGCCCAAGGACCCCGCAGGCCCGTTCTTCCAGTTGCGCTACCGGACTCCGGGGCCA |
| | CTACCGTCACCGCTTTTGCCAGGCTCCTCGTCACCCCTGGAGCCTCTAGATCTGGTGC |
| | TGCTGCGGCGCTTGGACCGAGAGGAGGCGGCGGCGCACCGGCTGCAGATCGAGGCATG |
| | GGACGGCGGCCGACCCCGGCGCACCGGCCTCCTGAGCGTGGAGCTGCGCGTGCTGGAT |
| | GAGAACGACAACCCGCCGGTCTTTGAGCAGGACGAGTACCGCGCCGCGGTGCGCGAGC |
| | ACGCCCAGCCGGGCGCCGAGGTCTGTCGCGTGCGCGCCACCGACCGCGACCTGGGGCC |
| | CAATGGCTTCGTGCGCTACAGCGTCCGCGCCCGGCAAGTGCCTGGGGCGGGTAGCGGC |
| | GGCGGGGCACTGGGCGACGCGGCCTACTTCGCGGTGGAGGAGCTGAGCGGCGTGGTGC |
| | GAGTGTGGAGACCTCTGGACCGCGAGGCACAGGCCTGGCACCAGTTGGTGGTGGAGGC |
| | CCGCGATGGAGGCGCCGAGCCTGAGGTTGCCACGGTCCGCGTGTCCATCGCCGTGCTG |
| | GACGTGAATGACAACCGGCCAGCAATTCACGTGCTCTTTCTCACAGAGGGAGGCGTCG |
| | CCCGTGTCTCTGAAGCCGCCCGACCGGGCGACTACGTGGCTCGCGTCTCGGTGTCTGA |
| | CGCGGACGGGGTAATAGGCAAAATTACAGCTATTGACATGGACTCTGGAAAGAATGGA |
| | CAGCTATTATATTTCCTTTTGTCTGATGGAAAATTCTTCAAGATGAATCCTAATACAG |
| | GAGAGTTAATCAATTGGGTGGCACTGGATCGTGAGCACCGGGCGCACCATGAGATGAC |
| | TGTGCTAGTGACAGACCGCGGCTCCCCACCACGAAACGCCACCATGGCGGTTTACGTC |
| | TCAGTTACTGACATCAATGATAACAGGCCCTTCTTCCCCCAGTGTCTCCCTGGAAAGG |
| | AGTTACACGTGAAGGTTCTGGAACGTCAACCAGTAAATATGTTGGTTACAACTGTGTT |
| | TGCAAAGGATCCTGATGAAGGAAATAATGCAGAAGTTACATACTCAGTATCTTCAGAA |
| | GATAGTTCTGATCACTTTAAGATTGACGCCAACAATGGTGAAATAAGAACAACCACAA |
| | TACTTTCGTATGATTATAGACCTTCCTACAGAATGAGTGTCATTCCCACTGACCAGGG |
| | AGTGCCTCCTCTTCAAGGACAGGCAGTTGTTAATATTCAGGAACTTGATTATGAGACG |
| | ACATCTCATTATCTTTTCAGAGTGATTACTACAGACCATAGCAAAAACCTTTCCCTGA |
| | GTAGCACAGTCTTCCTTAGTATCGATGTGGAAGATCAGAATGACCATTCCCCATCTTT |
| | CCAGGATGAGCTCATTGTGATCAGTGTAGAGGAGAATGTTCCCATAGGAACCCTGGTG |
| | TATGTCTTCAATGCCAAAGATGATGACGGCAGTTTTTTGAACAGTAGAATACAATACT |
| | ACATTGAATCCCACAACCCTGGCACGAATCCATTTCTCATCCACCCCTCATTTGGCAC |
| | ACTAGTCACTGTGTCCCGTCTTGACAGAGAAAGCATTCCAACTGTCATCCTGACAGTA |
| | ACAGCATCTGATCAGGCTGTGAATGTGACAGACCGGCGACTGAGATCACTGACAGCAC |
| | AAATAGTGATTTTGGATGTAAATGACCACAACCCCACTTTTATTTCTTTCCCCAATGC |
| | CCATGTCAAAGAGGATGTCACAGTGCGCTCCTTGGTCCACCACATAACTGCTCACGAT |
| | CCAGACGAAGGAAGGAATGGAAAAGTAACATACACCATCCTCTCAGGAAATGAAAACA |
| | TGACGTTTATGCTAGATGAGTCATCAGGCTTACTAACCACAACCTGTCCTTTGGATTA |
| | TGAAATGAAAACTCAGCATATTCTCACTGTTCTGGCACTGGATGATGGCACACCAGCA |
| | CTTTCTTCATCCCAGACTTTGACAGTTACTGTTCTTCATGTAAATGATGAAGCTCCAG |
| | TATTTAAGCAGCACCTGTATGAAGCCTCAGTGAAAGAAAACCAAAATCCAGGGGAGTT |
| | TGTTACCAGGGTTGAAGCTCTGGACAGAGATTCAGTGTTCCTTAATACTAGAGAGCTT |
| | AACATGTGTTTTCTAGCATTCTACGATGCAGTTTTTAAAAATGGTGGGCTAAGTGCCC |
| | AAGCCTTTGTTCGTGTGGACCTGGAGGACGTGAATGATAATCATCCTGTGTTTAACCC |
| | ATCAACCTATGTGACGAGCATCAGTGATGAGACCCAGCCAGGCACCGAGATCATCPAT |
| | GTTCTTGCCACTGACCAGGACTCTGGGATATATGGGACAGTCGCTTATGAGCTTATTC |
| | CAGGAAACGTGTCGTCCCTTTTTACCATTGACTCCACCACAGGAATTATTTACTTAAC |
| | ATTACCTCTTAGTCATTTGGAATCTACCACACTTTCGTTGATGGTCTCTGCTCAAGAC |
| | GGTGGTGGGCTCACAGCTGTCATTAATGCCGATGTCACCATACACATTTTCCAGACAA |
| | CTCTGGCACCTGCTGAGTTTGAAAGGCCTAAGTACACTTTCTTACTTTATGAAGATGT |
| | GCCTGAAGATAGTCCCATTGGAACAGTGAAAGCAAGAGAGCCCTTGAATCCACCTAGG |
| | AGCTCTGTAATACACCTGCAAGTTAGAGTTTTGGATGCCPATGACCACAGTCCTTCTT |
| | TTCCCACACTTTATTACCAGTCCTCTGTGAGAGAAGATGCTGAAGTGGGAACAGTGGT |
| | TCTTGTGCTTTCAGCTCTGGACAAGGATGAAGGCCTGAATGGGCAAACTGAGTATTTT |
| | CTGACTGATGAGGCTTGTGGTGCATTCACCATTGATCCTATGTCAGCCACATTGAAAA |
| | CCAGCAACACCCTCGACCGTGAAGCCAGATCTCACCATACATTTAGTGCTGTGGCCAG |
| | AGACTGTAGCATCCAGGGTTCACGAAGCACCACTGTAATTATAAAAGTATATGTCACT |
| | GATGTTAATGACAATGATCCAGTTTTGGAACAGAACCCTTTTCATGTGTTTCTTTCCC |
| | CCGAGTCGCCTACAAACCAGACAACTGTCATTGTGAGAGCTGATCACCTGGACTTGGG |
| | CCCCAATGGAACTGTGGACTCCGATGACTCCCCGCTGCTGGACGACTTCCACGTGCAC |
| | CCGGACACCGGCATCATCCGCACTGCCCGGCGCCTGGACCGCGAGCGCCGGGACCACT |
| | ACAGCTTCGTCGCCGCCACGCTGCTGGGCGCTGTGGTGCAGGTGGAGATTCGCCTCPA |
| | CGACGTGAATGACCACTCGCCCCGCTTTCCCCTCGACTCCCTGCAACTCGACGTCTCC |
| | GAGCTCAGCCCGCCAGGGACCGCCTTCCGCCTGCCAGTTGCCCACGATCCGGACGCCG |
| | GACTGTTCAGCACTCAGGGCTACACCCTGGTGCAACCGTCCGACCTGCCCAAGGACCC |
| | CGCAGGCCCGTTCTTCCAGTTGCGCTACCGGACTCCGGGGCCACTACCGTCACCGCTT |
| | TTGCCAGGCTCCTCGTCACCCCTGGAGCCTCTAGATCTGGTGCTGCTGCGGCGCTTGG |
| | ACCGAGAGGAGGCGGCGGCGCACCGGCTGCAGATCGAGGCATGGGACGCCGGCCGACC |
| | CCGGCGCACCGGCCTCCTGAGCGTGGAGCTGCGCGTGCTGGATGAGAACGACPACCCG |
| | CCGGTCTTTGAGCAGGACGAGTCCCGCGCCGCGGTGCGAGAGCACGCCCAGCCGGGCG |
| | CCGAGGTCTGTCGCGTGCGCGCCACCCCCCGCGACGTGGGGCCCAATGGCTTCGTGCG |
| | CTACAGCGTCCGCGCCCGGCAAGTGCCTGGGGCGGGTAGCGGCGGCGGGGCACTGGCC |
| | GACGCGGCCTACTTCGCGTTGGTGGTGGAGGCCCGCGATGGAGGCGCCGAGCATGAGG |
| | TTGCCGCGGTGCGTGTGTCCATCGCCGTGCTGGACGTGAATCACAACCGGCCAGCAAT |
| | TCACGTGCTCTTTCTCACACAGGGAGGTGTCGCCCGTGTCTCTGAAGGCGCCCCCCCG |
| | GGCGACTACGTGGCTCGCGTCTCGGTGTCTCACGCGGGCGGTGACTGGGAGAAGGAAG |
| | ATGAGGCCACAGGGGAGCTTGGTGTGACAGCCTCTGATGCAGATTCAGGACTCTATGG |
| | CTTTATTGAATATTCTCTTTATGATGGATTCCTGAGCTATCAAGCACCTCAGGCATTC |
| | CGGATCGACCCTCATGATGGGCAAATCTGTGTTTCTCAAGATATCGACAGGGAAAGGG |
| | ATCCAGCTACCTATGATCTCCTGGTGGAAGCTAAGGATGGGTTTGAAATCATGCCAGG |
| | TGCTTCATTTGAATTATTCGAGATAAATTCTGACACTGGAGAGGTAGTGACAACCACC |
| | ATACTTGACAGAGAAATTCAAGAAGTCTTCACCCTTCGAGTACTAGTACGAGATGGGG |
| | GATTCCCTTCATTGTCCAGCACCACAACAATCCTCTGCACTGTTGAAGATGAAAACGA |
| | TCACGCACCAGAGTTTATTGTTTCCAGTTATGACATTGAGGTTCTGGAAAACCAGGAA |
| | CCAGAGGTTGTCTATACGGTTTTACCCTCTGATATGGATGCTGGCAATAACAGAGCTG |
| | TTGAATATCACATAATTGACTCCTCAGAACCAATCTTTTACAGGATTTCTTCTGGTGA |
| | TCTCGGCGGAAAGTTCTCCATTCACCCGCGGCTGGGCACTATTCGCACCCGGAAGCCC |
| | CTGGATCACGAGACGCAGCCCGTGGTTGTGCTCACGGTGCAGGCGCAGCTCGGCAGCG |
| | CCCCAGCCTGCAGCAGCACCGAGGTCAACATAACAGTCATCGATGTCAATGACAACCA |
| | CCCACCGTTCCTCAGGACCTCGGATGAGATTAGAATATCCCAGACCACGCCCCCTGGC |
| | ACAGCCTTGTACCTCGCACGTGCGGAAGACAGAGACAGTGGGCGGAACGGACTCATCC |
| | GGTACTCCATCGCCAGCCCGCAGCCAGGCGTCTTTGCCATCGACAGAGCCCTGGGGGT |
| | GCTGTTCCTCAACGGCAGCCTGGGCGCGGGCGAGCAGCGGGAGCTCACGCTGACTCTC |
| | AGGGCCGAGGACCAAGGCGTGCATCCTCAGGCAGCCCTGCTGGTGCTGACAGTCGTTA |
| | TCGAGAAACGCGAACACAGCCCATCCTGGACTTTCGAACATTTGGTCTATCAAGTGGA |
| | AGTCAGTACTTCTATTGTGACTGTTAAAGCTTTTGCTCCTGACTCAATTCAGGACAGC |
| | ATGAAATATTCAATTTTTAGTGGAAATGAAGATGGAGTTCTTTCCCTGTGCTCTAAGT |
| | CAGGTGTGGTGAACTGCCTTGCTTCTCTCAGTCACACAGACTTTCTCTCCCTGAAATT |
| | TGAATCTTCGGTGAAGGGACACCAAGACAGAGACAAATTACAGCCAATTCATCTTGAT |
| | GACAACAACTCAAAGAAGCTGTGCTTTACATTCCCTAGAGCCACTCAGGCTCTTGTAT |
| | TCACTGGGCACTGTCTTTCTGATACATCTCTCCCCGGTTGGGTTTTTCCTACCGACTT |
| | GGACAGTGGTTTGAACGGCCTGATTGAGTATTCTATTCTGTCTGGCAACCAAGAAGAA |
| | GCATTCCAGATTGATGCACTGAGTGGTGTGATAACAACAAAAGCGATTCTAGATTACG |
| | AGCTCACCAGCTCCTACAGCTTGATTGTCCAAGCCACAGATAAAGGGATGCCCAGGCT |
| | TTCTAATACGACTGTAATCAAGGTACAGGTGACTGATATAAATGACAATGCCCCAGCT |
| | TTTCTCCCCTCTGAAGCAGTGGAAATTACAGAAGTCATGACTATTTCAGAAGATTCTT |
| | TGCCTGGTGTAATTGTGACTCATGTGTCAGTTCATGATGTGGATTTGAATTCAGCTTT |
| | CATATTCAGTTTTGCCAAAGAGAGTAATCCTGGAACCAAGTTTGCTATTGATCAGAAC |
| | ACTGGAGTGGTGGTGTTGGTGAAAACATTGGATTTTGAAGAAATGACTGAATATGAGC |
| | TGCTCATCCAAATTTCTGATTCAGTGCACTACACAGAGGGAGCACTTGTAGTCCGTGT |
| | GCTGGATGTCAATGATAATCCACCAGTGTTTTCTCAAGATTTCTATCAGGTCACAGTT |
| | CCTGAATCAATACCTGTGGCGTATTCAGTGCTGACTCTGTCAGCCACAGACTTAGAAA |
| | GCAATGAGAACATTTCTTACAGAATTCTATCCTCTTCTAAGGAATTCTCCATTGATCC |
| | TAAGAATGGCACAATATTTACTATCAGTCCCGTATTACTTCTGGATACAATATCAACA |
| | ACTCGATTTCTTGTGCAAGCCAGTGATGGTGGAAATCCTGACCCGAGAGCTCTTACTT |
| | TAGTGGAGATAGGAATAGAAGATATGAACAATTATGCCCCTGAATTCACAGTCAAATC |
| | CTATAATCTTAGCCTAAGTGAGCATGCTCTGGTTGGAAGCACGCTTGTTACATTTTCA |
| | AACATCOACCATGACTGGACCCGTGAAAACACATATGTTGAATATTCCATCATCAGTG |
| | GTAATTCACAGAACAATTTTCATGTGGAAACTAAGTTCTTTCATTCAGAATATCCTTA |
| | TAAGCAAGTCCGTTATCTTGTGTTGCTTCACAGTCTGGACAGAGAAGCAAGTGCTAGC |
| | CATGAGCTTGTCATTCTGGCATCTGACAGTGGCTGCCCTCCATTGAGTTCCACAGCTG |
| | TCATATCAATACAAGTACTTGATGTCAATGACAATCCCCCAAACTTCAGCAGCCTGAG |
| | CTATCACACCCATGTCAAGGAAAGCACCCCTCTAGGGAGTCACATCACTGTGGTCTCA |
| | GCAAATGACCGTGACACAGGGTCACATGCAGAAATCATCTACAACATCATCTCTGGAA |
| | ATGAGAAGGGACATTTTTACTTAGAAGAAAACACTGGAGTTCTTTATTTGATTAAACC |
| | TCTGGATTATGAAAAAATGACAAAATTCACCTTAACTGTCCAAGCTTCAGATGCAGAA |
| | AAGAAACATTTTTCTTTTGCAGTTGTGTTTGTCAGTGTCCTGGATGATAACGACCATG |
| | CACCTCAGTTTATGTTCTCAAGCTTCAGCTGTATTGTTCCAGAAAATCTGCCTATTTC |
| | CTCTACCATATGCTCTATAAATGCTCTGGATTTTGATGCTGGTCCGTATGGAGAATTG |
| | ACCTATTCTATTGTATCACCCTGTTTTCTCACTCATGGAATGTCTTATGATCATGATC |
| | TCTTCCTCATTGACCCTTTGACAGGGGATATTCATGCTAAGCAAATCCTTGACTATGA |
| | AAATGGCAATAAATACTGCCTCACAGTCCAAGCCAAAGACAAAGGTGATGCAACTGCC |
| | TCCTTAGTGGTCTGGCTGGATATTGAAGGGATAGATGAATTTGAGCCCATTTTCACTC |
| | AAGATCAGTATTTTTTCACCCTCCCAGAAAAGAATAAAGACAGACAGTTGATTGGCAG |
| | AGTGGAAGCCTCAGATGCAGATGCTGGTATTGATGGAGTCATTCTTTACTCCCTTGGA |
| | ACCTCATCTCCTTTCTTTTCAGTAAATAGAACCAATGGAAATATTTATTTGATTAGAG |
| | CCCTTCCCCTAATAAAAAGTCAACTCAACAAAGAAGACACCTTGGAAATGAAAATAAT |
| | CGCTCATAGTCCCAAATCAGATTCCAAGTTTGCATCTTGCACTGTTTTTGTGAATGTG |
| | TCTTTCTCCTCTGAAGGAACACCCTTGGCAGTGTTCGCCAGCAGCTTTTCAATCAGCC |
| | TGGTGGTCTCCTTTTTAGTGTTTCTGATACTCATCTGCATTCTAATTGTAATGATTTT |
| | AAGACATAAACAAAAAGACACAATAAACAATTATGAGGAGAAGAAAACCTCATCTTTA |
| | GATGCGGACTTGAGAGTGACCCGGGATGCCAGTGTGCTCAAAGCCTTCCAGPAAACTG |
| | ACGACTGCAGTAACGAGGTGGTCCCTGTGGATGCCACTCCCCAATCCTTCACTTTAAT |
| | AAGTATCATGGAGAAGGATATTGTCAATCTGTACAGATACTCAAACTCCAGTGGCCAC |
| | TGTTCTGTGGAAGGAGAAACTGCAGAAGATAAGGAAATCCAGAGGATAAATGAGCATC |
| | CCTACAGAAAGTGCTCAGACTCAGCTCTGAGTGACCACGAGTCCAGGGTGCCAGACTC |
| | GGGTATCCCGAGGGACTCACACCAGCTCTCCTGCCTATCTGGGGAAACTGATGTGATG |
| | GTGACTGCCGAAACAGCAGAAGCCAGCCAAACATTTGGGGAAGCAGATCAAGGGGAAG |
| | GCTGCAGCACCACCTGTGCTCAAAATAATGTGTTACCCCAGACAGTTCAGAAGAGAGA |
| | GGCAAAAGAGAGCATCCTGGCTGACGTTAGAAAAGAGTCTGTCTTTATTTCAGGTGAT |
| | CAGGAAGTAAGGTGTGCAGCTCTTTCAACTCAGACGACCTCTCATCATGATGGAAAAG |
| | ACAACTATCACTGGAATTATCTTCTTAGTTCGGAGCCCAAATTCCAACCTCTTGCCTC |
| | AGTATTTAATGATATTGCAAAACTAAAGGATGAACATTTGCATATGCCTGGCATTCCA |
| | AAAGAGAAGAAATCTTTTGTTTTTCCACCCCCTTTGATAACAGCAGTAGCCCAGCCTG |
| | GGATTAAAGCAGTCCCACCAAGAATGCCGCCAGTAAACCTGGGGCAGGTGCCTCCGAA |
| | ACACCCACGCTCTCCCATCCCCTACCATCTTGGTTCTCTGCCAGAAGGCATGACTCCC |
| | AATTTTTCTCCATCTCTTTCCCTATTGACGATGCAGCCTCCTGCCTTGTCTCCACTGT |
| | TGAGAGAAGGAGAATTATTAGGAACACACATCAGTGGTACATGCCATGAACTTAAAGC |
| | AGAAGATGAAGTTCAAATATGAAACCACTGGGATGCCAAGTACCTGCTCACCATTGGT |
| | CATGAATGAATGAACAAAATGTTTTCAAGCCGGCAACTCGAGATTGGGCTCATTTTTA |
| | TCTAAAAGCAAGTGATGTAATTTAGTTAGAGTTTTTAAAACTTCCCCATTAAAGTTTC |
| | TCCAATTTCAAAAAAAAAAAAAAAAAAAA |

| ORF Start: ATG at 1 | ORF Stop: TGA at 9010 | |
|---|---|---|
| SEQ ID NO: 126 | 3003 aa | MW at 329184.0 kD |

| | |
|---|---|
| NOV28b, | MEKCGLKEEGSYADIDPVAHDPDAGLFSTQGYTLVQPSDLPKDPAGPFFQLRYRTPGP |
| CG58567-05 Protein | LPSPLLPGSSSPLEPLDLVLLRRLDREEAAAHRLQIEAWDGGRPRRTGLLSVELRVLD |
| Sequence | ENDNPPVFEQDEYRAAVREDAQPGAEVCRVRATDRDLGPNGFVRYSVRARQVPGAGSG |
| | GGALGDAAYFAVEELSGVVRVWRPLDREAQAWHQLVVEARDGGAEPEVATVRVSIAVL |
| | DVNDNRPAIHVLFLTEGGVARVSEGARPGDYVARVSVSDADGVIGKITAIDMDSGKNG |
| | QLLYFLLSDGKFFKMNPNTGELINWVALDREHRGHHEMTVLVTDRGSPPRNATMAVYV |
| | SVTDINDNRPFFPQCLPGKELHVKVLEGQPVNMLVTTVFAKDPDEGNNAEVTYSVSSE |
| | DSSDHFKIDANNGEIRTTTILSYDYRPSYRMSVIATDQGVPPLQGQAVVNIQELDYET |
| | TSHYLFRVITTDHSKNLSLSSTVFLSIDVEDQNDHSPSFQDELIVISVEENVPIGTLV |
| | YVFNAKDDDCSFLNSRIQYYIESHNPGTNPFLIHPSFGTLVTVSRLDRESIPTVILTV |
| | TASDQAVNVTDRRLRSLTAQIVILDVNDHNPTFISFPNAHVKEDVTVGSLVHHITAHD |
| | PDEGRNGKVTYSILSGNENMTFMLDESSGLLTTTCPLDYEMKTQHILTVLALDDGTPA |
| | LSSSQTLTVTVLDVNDEAPVFKQHLYEASVKENQNPGEFVTRVEALDRDSVFLNTREL |
| | NMCFLAFYDAVFKNGGLSAQAFVRVDLEDVNDNHPVFNPSTYVTSISDETQPGTEIIN |
| | VLATDQDSGIYGTVAYELIPGNVSSLFTIDSTTGIIYLTLPLSHLESTTLSLMVSAQD |
| | GGGLTAVINADVTIHIFQTTLAPAEFERPKYTFLVYEDVPEDSPIGTVKAREPLNPPR |
| | SSVIHLQVRVLDANDHSPSFPTLYYQSSVREDAEVGTVVLVLSAVDKDECLNGQTEYF |
| | LTDEACGAFTIDPMSGTLKTSNTLDREARSQHTFSAVARDCSIQGSRSTTVIIKVYVT |
| | DVNDNDPVLEQNPFDVFLSPESPTNQTTVIVRADDLDLGPNGTVDSDDSPLLDDFHVH |
| | PDTGIIRTARRLDRERRDHYSFVAATLLGAVVQVEIRVNDVNDHSPRFPLDSLQLDVS |
| | ELSPPGTAFRLPVAHDPDAGLFSTQGYTLVQPSDLPKDPAGPFFQLRYRTPGPLPSPL |
| | LPGSSSPLEPLDLVLLRRLDREEAAAHRLQIEAWDGGRPRRTGLLSVELRVLDENDNP |
| | PVFEQDESRAAVREHAQPGAEVCRVRATARDVGPNGFVRYSVRARQVPGAGSGGGALG |
| | DAAYFALVVEARDGGAEHEVAAVRVSIAVLDVNDNRPAIHVLFLTEGGVARVSEGARP |
| | GDYVARVSVSDAGGDWEKEDEATGELGVTASDADSGLYGFIEYSLYDGFLSYEAPQAF |
| | RIDPHDGQTCVSQDIDRERDPATYDLLVEAKDGFEIMPGASFELFEINSDTGEVVTTT |
| | ILDREIQEVFTLRVLVRDGGFPSLSSTTTILCTVEDENDHAPEFIVSSYDIEVLENQE |
| | PEVVYTVLASDMDAGNNRAVEYHIIDSSEPIFYRISSGDLCGKFSIHPRLGTIRTRKP |
| | LDHETQPVVVLTVQAQLGSAPACSSTEVNITVMDVNDNHPAFLRTSDEIRISQTTPPG |
| | TALYLAPAEDRDSGRNGLIRYSIASPQPGVFAIDRALGVLFLNGSLGAGEQRELTLTL |
| | RAEDQGVHPQAALLVLTVVIEKREHSPSWTFEHLVYQVEVSTSIVTVKAFAPDSIQDS |
| | MKYSIFSGNEDGVLSLCSKSGVVNCLASLSHTDFLSLKFESSvKGHQDRDKLQPIHLD |
| | DNNSKKLCFTFPRATQALVFTGHCLSDTSLPGWVFATDLDSGLNGLIEYSILSGNQEE |
| | AFQIDALSGVITTKAILDYELTSSYSLIVQATDKGMPRLSNTTVIKVQVTDINDNAPA |
| | FLPSEAVEITEVMTISEDSLPGVIVTHVSVHDVDLNSAFIFSFAKESNPGTKFAIDQN |
| | TGVVVLVKTLDFEEMTEYELLIQISDSVHYTEGALVVRVLDVNDNPPVFSQDFYQVTV |
| | PESIPVGYSVLTLSATDLESNENISYRILSSSKEFSIDPKNGTIFTISPVLLLDTIST |
| | TRFLVEASDGGNPDPRALTLVEIGIEDMNNYAPEFTVKSYNLSLSEDALVGSTLVTFS |
| | NIDHDWTRENTYVEYSIISGNSQNNFHVETKFFHSEYPYKQVGYLVLLHSLDREASAS |
| | HELVILASDSGCPPLSSTAVISIQVLDVNDNPPNFSSLSYHTHVKESTPLGSHITVVS |
| | ANDRDTGSHAEIIYNIISGNEKGHFYLEENTGVLYLIKPLDYEKMTKFTLTVQASDAE |
| | KKHFSFAVVFVSVLDDNDHAPQFMFSSFSCIVPENLPISSTICSINALDFDAGPYGEL |
| | TYSIVSPCFLTHGMSYDHDLFLIDPLTGDIHAKQILDYENGNKYCLTVQAKDKGDATA |
| | SLVVWVDIEGIDEFEPIFTQDQYFFTLPEKNKDRQLIGRVEASDADAGIDGVILYSLG |
| | TSSPFFSVNRTNGNIYLIRALPLIKSQLNKEDTLEMKIIAHSPKSDSKFASCTVFVNV |
| | SFSSEGTPLAVFASSFSISLVVSFLVFLILICILIVMILRHKQKDTINNYEEKKTSSL |
| | DADLRVTRDASVLKAFQKTDDCSNEVVPVDATPEWLSLISIMEKDIVNLYRYSNSSGH |
| | CSVEGETAEDKEIQRINEHPYRKCSDSALSDHESRVPDSGIPRDSDQLSCLSGETDVM |
| | VTAETAEASQTFGEGDQGEGCSTTCAQNNVLPQTVQKREAKESILADVRKESVFISGD |
| | QEVRCAALSTQTTSDHDGKDNYHWNYLLSWEPKPQPLASVFNDIAKLKDEHLHMPGIP |
| | KEKKSFVFPPPLITAVAQPGIKAVPPRMPAVNLGQVPPKHPRSPIPYHLGSLPEGMTP |
| | NFSPSLSLLTMQPPALSPLLREGELLGTHISGTCHELKAEDEVQI |

| | |
|---|---|
| SEQ ID NO: 127 | 10267 bp |

| | |
|---|---|
| NOV28c, | ATGGAGAAATGTGGGCTTAAAGAGGAAGGCAGTTACGCGGACATAGATCCAGTTGCCC |
| CG58567-06 DNA | ACGATCCGGACGCCCCACTCTTCAGCACTCAGGGCTACACCCTGGTGCAACCGTCCGA |
| Sequence | CCTGCCCAAGGACCCCGCAGGCCCGTTCTTCCAGTTGCGCTACCGGACTCCGGGGCCA |
| | CTACCGTCACCGCTTTTGCCAGGCTCCTCGTCACCCCTGGAGCCTCTAGATCTGGTGC |
| | TGCTGCGGCGCTTGGACCGAGAGGAGGCGGCGGCGCACCGGCTGCAGATCGAGGCATG |
| | GGACGGCGGCCGACCCCGGCGCACCGGCCTCCTGAGCGTGGAGCTGCGCGTGCTGGAT |
| | GAGAACGACAACCCGCCGGTCTTTGAGCAGGACGAGTACCGCGCCGCGGTGCGCGAGG |
| | ACGCCCAGCCGGGCGCCGAGGTCTGTCGCGTGCGCGCCACCGACCGCGACCTGGGGCC |
| | CAATGGCTTCGTGCGCTACAGCGTCCGCGCCCGGCAAGTGCCTGGGGCGGGTAGCGGC |
| | GGCGGGGCACTGGGCGACGCGGCCTACTTCGCGGTGGAGGAGCTGAGCGGCGTGGTGC |
| | GAGTGTGGAGACCTCTGGACCGCGAGGCACAGCCCTGGCACCAGTTGGTGGTGGAGGC |
| | CCGCGATGGAGGCGCCGAGCCTGAGGTTGCCACGGTGCGCGTGTCCATCGCCCTGCTG |
| | GACGTGAATGACAACCCGCCAGCAATTCACGTGCTCTTTCTCACAGAGGGAGGCGTCG |
| | CCCGTGTCTCTGAAGGCGCCCGACCGGGCGACTACGTGGCTCGCGTCTCGGTGTCTGA |
| | CGCGGACGGTGACTGGGAGAAGGAAGATGAGGCCACAGGGGAGCTTCGTGTGGGTCTT |
| | GGAGACGGGAGCATCTCTCTGTCCTTGCAAGGCGGAGAGGGAGACTTCGCGTTGCTAC |
| | CCGGCGGCCCCCCAGGGGTATTTTTCCTTTGCCTGGAGGGGCCCCTGGACAGAGAGAG |
| | CCGCGATCTGTATGAGTTACTACTGGTGGCCACGCACGCGGGGTCCCCGCCGCTGAGC |
| | ACGGAGGAGACGCTGCTACTCCGGGTCGCTGACCTCAATGACCAACCACCTCTCTTCA |
| | GCCAACAGCATTACAAGGCCTCAGTGTCCGAGGCCGCCGCCCCTGGCACTGTAGTCAT |
| | GTGGGTCAGCGCCTCCGATGCCGACGAGGCAGGCAGTGATCACGCCTGGCTCCGCTAC |
| | ACTGTAGTCCAACTCTCGGCTCCCTGCAATCTCGGCTCCCTGCAATCAAAGATGGTCC |
| | ACACCGCAGAGTGTGGACCATCTTTTGCCATTGATTCCGAAAGCGGTGCGATCAGCAC |
| | TATCCGGACTCTAGACCGAGAGGTCCAGGAGGCGGTGGAGCTGAAAGTGCTGGCCCAG |
| | GACCTCGGAGAGCCCCCACTCTCTGCCACCTGCCTGGTGAGCATCACCGTAGATGATG |
| | TGAATGACAATGAGCCCATCTTCTGGAGGCAGGTGTACAATGCCACCATTGCAGAGCA |
| | TGCCCCGGTTGGACACTGCTTTCTGCAGCTTATATCTGCTCAAGTTGCCTCTGTCAAA |
| | ATCAAACACAAACACAAGGAGATACACGAGAAACACAATCTTGCCTATATTTCCTGTC |
| | CAGCACGCACCATCTATGTCATAACCTGGGCAGATGGTGCTGCTGCCTTTACTGGGAC |
| | AGACTTTGCATTCAGTTCTGATGAACTTCAAGCCTTTGTTCTCAAGTCTCTGTTCTGT |
| | GAATTAGGAGAAGGAGAGTTAATCAATTGGGTGGCACTGGATCGTGAGCACCGGGGGC |
| | ACCATGAGATGACTGTGCTAGTGACAGACCGCGGCTCCCCACCACGAAACGCCACCAT |
| | CGCGCTTTACGTCTCAGTTACTCACATCAATGATAACAGGCCCTTCTTCCCCCAGTGT |
| | CTCCCTGGAAAGGAGTTACACGTGAAGGTTCTGGAAGGTCAACCAGTAAATATGTTGG |
| | TTACAACTGTGTTTGCAAAGGATCCTGATGAAGGAAATAATGCAGAAGTTACATACTC |
| | AGTATCTTCAGAAGATAGTTCTGATCACTTTAAGATTGACGCCAACAATGGTGAAATA |
| | AGAACAACCACAATACTTTCGTATGATTATAGACCTTCCTACAGAATGAGTGTCATTG |
| | CCACTGACCAGGGAGTGCCTCCTCTTCAAGGACAGGCAGTTGTTAATATTCAGGTGAT |
| | CCCACTATCCAAAGGGAGAGCAATCATGTCTCAGAATATTAGACATTTAATTATACCA |
| | GAAAATTTGAAGCCCACAAAAATAATGAGCTTGATAAAGTCATCTGATCACCTTCAAC |
| | AACATTATAATGGAAAGTTACATTTTAGTATTGTTGCAGATGATAAGGATGGACACTT |
| | TGAAATAGACAGCTCAACCGGAGACTTGTTTCTTTCTAACGAACTTCATTATGAGACG |
| | ACATCTCATTATCTTTTCAGAGTGATTACTACAGACCATAGCAAAAACCTTTCCCTGA |
| | GTAGCACAGTCTTCCTTAGTATCGATGTGGAAGATCAGAATGACCATTCCCCATCTTT |
| | CCAGGATGAGCTCATTGTGATCAGTGTAGAGGAGAATGTTCCCATAGGAACCCTGGTG |
| | TATGTCTTCAATGCCAAAGATGATGACGGCAGTTTTTTGAACAGTAGAATACAATACT |
| | ACATTGAATCCCACAACCCTCGCACGAATCCATTTCTCATCCACCCCTCATTTGGCAC |
| | ACTAGTCACTGTCTCCCGTCTTGACAGAGAAAGCATTCCAACTGTCATCCTGACAGTA |
| | ACAGCATCTGATCAGGCTGTGAATGTGACAGACCGGCGACTGAGATCACTGACAGCAC |
| | AAATAGTGATTTTGGATGTAAATGACCACAACCCCACTTTTATTTCTTTCCCCAATGC |
| | CCATGTCAAAGAGGATGTCACAGTGGGCTCCTTGGTCCACCACATAACTGCTCACGAT |
| | CCAGACGAAGGAAGGAATGGAAAAGTAACATACAGCATCCTCTCAGGAAATGAAAACA |
| | TGACGTTTATGCTACATGAGTCATCAGGCTTACTAACCACAACCTGTCCTTTGGATTA |
| | TGAAATGAAAACTCAGCATATTCTGACTGTTCTGGCACTGGATGATGGCACACCAGCA |
| | CTTTCTTCATCCCAGACTTTGACAGTTACTGTTCTTGATGTAAATGATGAAGCTCCAG |
| | TATTTAAGCAGCACCTGTATGAAGCCTCAGTGAAAGAAAACCAAAATCCAGGGGAGTT |
| | TGTTACCAGGGTTGAAGCTCTGGACAGAGATTCAGGTATGAGGCTGAATGGAGATCCA |
| | GACAGGGAGCTGTGTGCAGGAGGGAACCCTCTTGGAAGCAGGGCCCCTCCTGGAAGCA |
| | GGACCCCTCCTGAAGGTGGGCTAAGTGCCCAAGCCTTTGTTCGTGTGGACCTGGAGGA |
| | CGTGAATGATAATCATCCTGTGTTTAACCCATCAACCTATGTGACGAGCATCAGTGAT |
| | GAGACCCAGCCAGGCACCGAGATCATCAATGTTCTTGCCACTGACCAGGACTCTGGGA |
| | TATATGGGACAGTGGCTTATGAGCTTATTCCAGCAAACGTGTCGTCCCTTTTTACCAT |
| | TGACTCCACCACAGGAATTATTTACTTAACATTACCTCTTAGTCATTTGGAATCTACC |
| | ACACTTTCGTTGATGGTCTCTGCTCAAGACGGTGGTGGGCTCACAGCTGTCATTAATG |
| | CCGATCTCACCATACACATTTTCCAGACAACTCTGGCACCTGCTGAGTTTGAAAGGCC |
| | TAAGTACACTTTCTTAGTTTATGAAGATGTGCCTGAAGATAGTCCCATTGGAACAGTG |
| | AAAGCAAGAGAGCCCTTGAATCCACCTAGGAGCTCTGTAATACACCTCCAAGTTAGAG |
| | TTTTGGATGCCAATGACCACAGTCCTTCTTTTCCCACACTTTATTACCAGTCCTCTGT |
| | GAGAGAAGATGCTGAAGTGGGAACAGTGGTTCTTGTGCTTTCAGCTGTGGACAAGGAT |
| | GAAGGCCTGAATGGGCAAACTGAGTATTTTCTGACTGATGAGGCTTGTGGTGCATTCA |
| | CCATTGATCCTATGTCAGGCACATTGAAAACCAGCAACACCCTCGACCGTGAAGCCAG |
| | ATCTCAGCATACATTTAGTGCTGTGGCCAGAGACTGTAGCATCCAGGGTTCACGAAGC |
| | ACCACTGTAATTATAAAAGTATATGTCACTGATGTTAATGACAATGATCCAGTTTTGG |
| | AACAGAACCCTTTTGATGTGTTTCTTTCCCCCCAGTCGCCTACAAACCAGACAACTGT |
| | CATTGTGAGAGCTGATGACCTGGACTTGGGGCCCAATGGAACTGTGGACTCCGATGAC |
| | TCCCCGCTGCTGGACGACTTCCACGTGCACCCGGACACCGCCATCATCCGCACTGCGC |
| | GGCGCCTGGACCGCGAGCGGCGGGACCACTACAGCTTCGTCGCCGCCACGCTGCTGGG |
| | CGCTGTGCTGCAGGTGGAGATTCGCGTCAACGACGTGAATGACCACTCCCCCCGCTTT |
| | CCCCTCGACTCCCTGCAACTCGACGTCTCCGAGCTCAGCCCGCCAGGGACCCCCTTCC |
| | GCCTGCCAGTTGCCCACGATCCGGACGCCGGACTGTTCAGCACTCAGGGCTACACCCT |
| | GGTGCAACCGTCCGACCTGCCCAAGGACCCCGCAGGCCCGTTCTTCCAGTTGCGCTAC |
| | CGGACTCCGCGGCCACTACCGTCACCGCTTTTGCCAGGCTCCTCGTCACCCCTGGAGC |
| | CTCTAGATCTGGTGCTGCTGCGGCCCTTGGACCCAGAGGAGGCGGCGCCCCACCGGCT |
| | GCAGATCGAGGCATGGGACGGCGGCCGACCCCGGCCCACCGGCCTCCTGAGCGTGGAG |
| | CTGCGCGTGCTGGATGAGAACGACAACCCGCCGGTCTTTGAGCACGACGAGTCCCGCG |
| | CCGCGGTGCGAGAGCACGCCCAGCCGGGCGCCGAGGTCTGTCGCGTCCCCGCCACCGC |
| | CCGCGACGTGGGGCCCAATGGCTTCGTGCGCTACAGCGTCCGCGCCCGGCAAGTGCCT |
| | GGGGCGGGTAGCGGCGGCGGGGCACTGGGCGACGCGGCCTACTTCGCGTTGGTGGTGG |
| | AGGCCCGCGATGGAGGCGCCGAGCATGAGGTTGCCGCGGTGCGTGTGTCCATCGCCGT |
| | GCTGGACGTGAATGACAACCGGCCAGCAATTCACGTGCTCTTTCTCACAGAGGGAGGT |
| | GTCGCCCGTGTCTCTGAAGGCGCCCGCCCGGGCGACTACGTGGCTCGCGTCTCGGTGT |
| | CTGACGCGGGCGGTGACTGGGAGAAGGAAGATGAGGCCACAGGGGACCTTCCTCTCAC |
| | AGCCTCTGATGCAGATTCAGGACTCTATGGCTTTATTGAATATTCTCTTTATGATGGA |
| | TTCCTGAGCTATGAAGCACCTCAGGCATTCCGGATCGACCCTCATGATGGGCAAATCT |
| | GTGTTTCTCAACATATCGACAGGGAAAGGGATCCAGCTACCTATGATCTCCTGGTGGA |
| | AGCTAAGGATGGGTTTGAAATCATCCCACGTGCTTCATTTGAATTATTCGAGATAAAT |
| | TCTGACACTGGAGAGGTAGTGACAACCACCATACTTGACAGAGAAATTCAAGAAGTCT |
| | TCACCCTTCGAGTACTAGTACGAGATGGGGGATTCCCTTCATTGTCCAGCACCACAAC |
| | AATCCTCTGCACTCTTGAAGATGAAAACGATCACGCACCAGAGTTTATTGTTTCCAGT |
| | TATGACATTGAGGTTCTGGAAAACCAGGAACCAGAGGTTGTCTATACGGTTTTAGCCT |
| | CTGATATGGATGCTGGCAATAACAGAGCTGTTGAATATCACATAATTCACTCCTCAGA |
| | ACCAATCTTTTACAGGATTTCTTCTGGTGATCTCGGCGGAAAGTTCTCCATTCACCCG |
| | CGGCTGGGCACTATTCGCACCCGGAAGCGCCTCGATCACGAGACGCAGCCCGTGGTTG |
| | TGCTCACGGTGCAGGCGCAGCTCGGCAGCGCCCCAGCCTGCAGCAGCACCGAGGTCAA |
| | CATAACAGTCATGGATGTCAATGACAACCACCCAGCGTTCCTCAGGACCTCGGATGAG |
| | ATTAGAATATCCCAGACCACGCCCCCTGGCACAGCCTTGTACCTCGCACGTGCGGAAG |
| | ACAOAGACAGTGGGCGGAACGGACTCATCCGGTACTCCATCGCCAGCCCGCAGCCAGG |
| | CGTCTTTGCCATCGACAGAGCCCTGGGGGTGCTGTTCCTCAACGGCAGCCTGGCCGCG |
| | GGCGAGCAGCGGGAGCTCACGCTGACTCTCAGCGCCGAGGACCAAGGCGTGCATCCTC |
| | AGGCAGCCCTGCTGGTGCTGACAGTCGTTATCGAGAAACGCGAACACAGCCCATCCTG |
| | GACTTTCGAACATTTGGTCTATCAAGTGGAAGTCAGTACTTCTATTGTGACTGTTAAA |
| | GCTTTTGCTCCTGACTCAATTCAGCACAGCATGAAATATTCAATTTTTAGTGGAAATG |
| | AAGATGGAGTTCTTTCCCTGTGCTCTAAGTCAGGTGTGGTGAACTGCCTTGCTTCTCT |
| | CAGTCACACAGACTTTCTCTCCCTGAAATTTGAATCTTCGGTGAAGCGACACCAAGAC |
| | AGAGACAAATTACAGCCAATTCATCTTGATGACAACAACTCAAAGAAGCTGTGCTTTA |
| | CATTCCCTAGAGCCACTCAGGCTCTTGTATTCACTGCGCACTGTCTTTCTGATACATC |
| | TCTCCCCGGTTGGGTTTTTGCTACCGACTTGGACAGTGGTTTGAACGGCCTGATTGAG |
| | TATTCTATTCTGTCTOGCAACCAAGAAGAAGCATTCCAGATTGATGCACTGAGTGGTG |
| | TGATAACAACAAAACCGATTCTAGATTACGAGCTCACCAGCTCCTACAGCTTGATTGT |
| | CCAAGCCACAGATAAAGGGATGCCCAGGCTTTCTAATACGACTGTAATCAAGGTACAG |
| | GTGACTGATATAAATGACAATGCCCCAGCTTTTCTCCCCTCTGAAGCAGTGGAAATTA |
| | CAGAACTCATGACTATTTCAGAAGATTCTTTGCCTGGTGTAATTGTGACTCATCTGTC |
| | AGTTCATGATGTGGATTTGAATTCAGCTTTCATATTCAGTTTTGCCAAAGAGAGTAAT |
| | CCTGGAACCAAGTTTGCTATTGATCAGAACACTGGAGTGGTGGTGTTGGTGAAAACAT |
| | TGGATTTTGAAGAAATGACTGAATATGAGCTGCTCATCCAAATTTCTGATTCAGTGCA |
| | CTACACAGAGGGAGCACTTGTAGTCCGTGTGCTGGATGTCAATGATAATCCACCAGTG |
| | TTTTCTCAAGATTTCTATCAGGTCACAGTTCCTGAATCAATACCTGTGGGGTATTCAG |
| | TGCTGACTCTGTCAGCCACAGACTTAGAAAGCAATGAGAACATTTCTTACAGAATTCT |
| | ATCCTCTTCTAAGGAATTCTCCATTGATCCTAAGAATGGCACAATATTTACTATCAGT |
| | CCCGTATTACTTCTGGATACAATATCAACAACTCGATTTCTTGTGGAAGCCAGTGATG |
| | GTGGAAATCCTGACCCGAGAGCTCTTACTTTAGTGGAGATAGGAATAGAAGATATGAA |
| | CAATTATCCCCCTGAATTCACAGTCAAATCCTATAATCTTAGCCTAAGTGAGGATGCT |
| | CTGGTTGGAAGCACGCTTGTTACATTTTCAAACATCGACCATGACTGGACCCGTGAAA |
| | ACACATATGTTGAATATTCCATCATCAGTGGTAATTCACAGAACAATTTTCATGTGGA |
| | AACTAAGTTCTTTCATTCAGAATATCCTTATAAGCAAGTCGGTTATCTTGTGTTCCTT |
| | CACAGTCTGGACAGAGAAGCAAGTGCTAGCCATGAGCTTGTCATTCTGGCATCTGACA |
| | GTCGCTGCCCTCCATTGAGTTCCACAGCTGTCATATCAATACAAGTACTTGATGTCAA |
| | TGACAATCCCCCAAACTTCAGCAGCCTGAGCTATCACACCCATGTCAAGGAAAGCACC |
| | CCTCTAGGGAGTCACATCACTGTGGTCTCAGCAAATGACCGTGACACAGGGTCACATG |
| | CAGAAATCATCTACAACATCATCTCTGGAAATGAGAAGGGACATTTTTACTTAGAAGA |
| | AAACACTGGAGTTCTTTATTTGATTAAACCTCTGGATTATGAAAAAATGACAAAATTC |
| | ACCTTAACTGTCCAAGCTTCAGATGCAGAAAAGAAACATTTTTCTTTTGCAGTTGTGT |
| | TTGTCAGTGTCCTGGATGATAACGACCATGCACCTCAGTTTATGTTCTCAAGCTTCAG |
| | CTGTATTGTTCCAGAAAATCTGCCTATTTCCTCTACCATATGCTCTATAAATGCTCTG |
| | GATTTTGATGCTGGTCCGTATGGAGAATTGACCTATTCTATTGTATCACCCTGTTTTC |
| | TCACTCATGCAATGTCTTATGATCATGATCTCTTCCTCATTGACCCTTTGACAGGGGA |
| | TATTCATGCTAAGCAAATCCTTGACTATGAAAATGGCAATAAATACTGCCTCACAGTC |
| | CAAGCCAAAGACAAACCTGATCCAACTCCCTCCTTACTCCTCTCCCTCCATATTGAAG |
| | GGATAGATGAATTTGAGCCCATTTTCACTCAAGATCAGTATTTTTTCACCCTCCCAGA |
| | AAAGAATAAAGACAGACAGTTGATTGGCAGAGTGGAAGCCTCAGATGCAGATGCTGGT |
| | ATTGATGCAGTCATTCTTTACTCCCTTGGAACCTCATCTCCTTTCTTTTCAGTAAATA |
| | GAACCAATGGAAATATTTATTTGATTAGAGCCCTTCCCCTAATAAAAAGTCAACTCAA |
| | CAAAGAAGACACCTTGGAAATGAAAATAATCGCTCATACTCCCAAATCAGATTCCAAG |
| | TTTGCATCTTGCACTGTTTTTGTGAATGTGTCTTTCTCCTCTGAAGGAACACCCTTGG |
| | CAGTGTTCGCCAGCAGCTTTTCAATCAGCCTGGTGGTCTCCTTTTTAGTGTTTCTCAT |
| | ACTCATCTGCATTCTAATTGTAATGATTTTAAGACATAAACAAAAAGACACAATAAAC |
| | AATTATGAGGAGAAGAAAACCTCATCTTTAGATGCGGACTTGAGAGTGACCCGGGATG |
| | CCAGTGTGCTCAAAGCCTTCCAGAAAACTGACGACTGCAGTAACGAGGTGGTCCCTGT |
| | GGATGCCACTCCGGAATGGTTGAGTTTAATAAGTATCATGGAGAAGGATATTGTCAAT |
| | CTGTACAGATACTCAAACTCCAGTGGCCACTGTTCTGTGGAAGGAGAAACTGCAGAAG |
| | ATAAGGAAATCCAGAGGATAAATGAGCATCCCTACAGAAAGTGCTCAGACTCAGCTCT |
| | GAGTGACCACGAGTCCAGGGTGCCAGACTCGGGTATCCCGAGGCACTCAGACCAGCTC |
| | TCCTGCCTATCTGCGGAAACTGATGTGATGGTGACTGCCGAAACAGCAGAACCCAGCC |
| | AAACATTTGGGGAAGGAGATCAAGGGGAAGGCTGCAGCACCACCTGTGCTCAAAATAA |
| | TGTGTTACCCCAGACAGTTCAGAAGAGAGAGGCAAAAGAGAGCATCCTGGCTGACGTT |
| | AGAAAAGAGTCTGTCTTTATTTCAGGTGATCAGGAAGTAAGGTGTGCAGCTCTTTCAA |
| | CTCAGACGACCTCTGATCATGATGGAAAAGACAACTATCACTGGAATTATCTTCTTAG |
| | TTGGGAGCCCAAATTCCAACCTCTTGCCTCAGTATTTAATGATATTGCAAAACTAAAG |
| | GATGAACATTTGCATATGCCTGGCATTCCAAAAGAGAAGAAATCTTTTGTTTTTCCAC |
| | CCCCTTTGATAACAGCAGTAGCCCAGCCTGGGATTAAAGCAGTCCCACCAAGAATGCC |
| | GGCAGTAAACCTGGGGCAGGTGCCTCCGAAACACCCACGCTCTCCCATCCCCTACCAT |
| | CTTGGTTCTCTGCCAGAAGGCATGACTCCCAATTTTTCTCCATCTCTTTCCCTATTGA |
| | CCATGCAGCCTCCTGCCTTGTCTCCACTGTTGAGAGAAGGAGAATTATTAGGAACACA |
| | CATCAGTGGTACATGCCATGAACTTAAAGCAGAAGATGAAGTTCAAATATGAAACCAC |
| | TGGGATGCCAAGTACCTGCTCACCATTGGTCATGAATGAATGAACAAAATGTTTTCAA |
| | GCCGGCAACTCGAGATTGGGCTCATTTTTATCTAAAAGCAAGTGATGTAATTTAGTTA |
| | GAGTTTTTAAAACTTCCCCATTAAAGTTTCTCCAATTTCAAAAAAAAAAAAAAAAAAA |
| | A |

| | ORF Start: ATG at 1 | ORF Stop: TGA at 10084 | |
|---|---|---|---|
| | SEQ ID NO: 128 | 3361 aa | MW at 367309.1 kD |
| NOV28c, | MEKCGLKEEGSYADIDPVAHDPDAGLFSTQGYTLVQPSDLPKDPAGPFFQLRYRTPGP | | |
| CG58567-06 Protein | LPSPLLPGSSSPLEPLDLVLLRRLDREEAAAHRLQIEAWDGGRPRRTGLLSVELRVLD | | |
| Sequence | ENDNPPVFEQDEYRAAVREDAQPGAEVCRVRATDRDLGPNGFVRYSVRARQVPGACSG | | |
| | GGALGDAAYFAVEELSGVVRVWRPLDREAQAWHQLVVEARDGGAEPEVATVRVSIAVL | | |
| | DVNDNRPAIHVLFLTEGGVARVSEGARPGDYVARVSVSDADGDWEKEDEATGELGVGL | | |
| | GDGSISLSLEGGEGDFALLPGGPPGVFFLCVEGPLDRESRDLYELLLVATDAGSPPLS | | |
| | TEETLLLRVADLNDQPPLFSQQHYKASVSEAAAPGTVVMWVSASDADEAGSDHAWLRY | | |
| | TVVQLSAPCNLGSLQSKMVHTAECGPSFAIDSESGAISTIRTLDPEVQEAVELKVVAQ | | |
| | DLGEPPLSATCLVSITVDDVNDNEPIFWRQVYNATIAEHAPVGHCFLQLISAQVASVK | | |
| | IKHKHKEIHEKHNLAYISCPAGTIYVITWADGAAAFSGTDFAFSSDELQAFVLKSLFC | | |
| | ELGEGELINWVALDREHRGHHEMTVLVTDRGSPPRNATMAVYVSVTDINDNRPFFPQC | | |
| | LPGKELHVKVLEGQPVNMLVTTVFAKDPDEGNNAEVTYSVSSEDSSDHFKIDANNGEI | | |
| | RTTTILSYDYRPSYRMSVIATDQGVPPLQGQAVVNIQVIPLSKGRAIMSQNIRHLIIP | | |
| | ENLKPTKIMSLIKSSDHLQQHYNGKLHFSIVADDKDGHFEIDSSTGDLFLSKELDYET | | |
| | TSHYLFRVITTDHSKNLSLSSTVFLSIDVEDQNDHSPSFQDELIVISVEENVPIGTLV | | |
| | YVFNAKDDDGSFLNSRIQYYIESHNPGTNPFLIHPSFGTLVTVSRLDRESIPTVILTV | | |
| | TASDQAVNVTDRRLRSLTAQIVILDVNDHNPTFISFPNAHVKEDVTVGSLVHHITAHD | | |
| | PDEGRNGKVTYSILSGNENMTFMLDESSGLLTTTCPLDYEMKTQHILTVLALDDGTPA | | |
| | LSSSQTLTVTVLDVNDEAPVFKQHLYEASVKENQNPGEFVTRVEALDRDSGMRLNGDP | | |
| | DRELCAGGNPLGSRAPPGSRTPPEGGLSAQAFVRVDLEDVNDNHPVFNPSTYVTSISD | | |
| | ETQPGTEIINVLATDQDSGIYGTVAYELIPGNVSSLFTIDSTTGIIYLTLPLSHLEST | | |
| | TLSLMVSAQDGGGLTAVINADVTIHIFQTTLAPAEFERPKYTFLVYEDVPEDSPIGTV | | |
| | KAREPLNPPRSSVIHLQVRVLDANDHSPSFPTLYYQSSVREDAEVGTVVLVLSAVDKD | | |
| | EGLNGQTEYFLTDEACGAFTIDPMSGTLKTSNTLDREARSQHTFSAVARDCSIQGSRS | | |
| | TTVIIKVYVTDVNDNDPVLEQNPEDVELSPESPTNQTTVIVRADDLDLGPNGTVDSDD | | |
| | SPLLDDFHVHPDTGIIRTARRLDRERRDHYSFVAATLLGAVVQVEIRVNDVNDHSPRF | | |
| | PLDSLQLDVSELSPPGTAFRLPVAHDPDAGLFSTQGYTLVQPSDLPKDPAGPFFQLRY | | |
| | RTPGPLPSPLLPGSSSPLEPLDLVLLRRLDREEAAAHRLQIEAWDGGRPRRTGLLSVE | | |
| | LRVLDENDNPPVFEQDESRAAVREHAQPGAEVCRVRATARDVGPNGFVRYSVRARQVP | | |
| | GAGSGGGALGDAAYFALVVEARDGGAEHEVAAVRVSIAVLDVNDNRPAIHVLFLTEGG | | |
| | VARVSEGARPGDYVARVSVSDAGGDWEKEDEATGELGVTASDADSGLYGFIEYSLYDG | | |
| | FLSYEAPQAFRIDPHDGQICVSQDIDRERDPATYDLLVEAKDGFEIMPGASFELFEIN | | |
| | SDTGEVVTTTILDREIQEVFTLRVLVRDGGFPSLSSTTTILCTVEDENDHAPEFIVSS | | |
| | YDIEVLENQEPEVVYTVLASDMDAGNNRAVEYHIIDSSEPIFYRISSGDLGGKFSIHP | | |
| | RLGTIRTRKPLDHETQPVVVLTVQAQLGSAPACSSTEVNITVMDVNDNHPAFLRTSDE | | |
| | IRISQTTPPGTALYLARAEDRDSGRNGLIRYSIASPQPGVFAIDRALGVLFLNGSLGA | | |
| | GEQRELTLTLRAEDQGVHPQAALLVLTVVIEKREHSPSWTFEHLVYQVEVSTSIVTVK | | |
| | AFAPDSIQDSMKYSIFSGNEDGVLSLCSKSGVVNCLASLSHTDFLSLKFESSVKGHQD | | |
| | RDKLQPIHLDDNNSKKLCFTFPRATQALVFTGHCLSDTSLPGWVFATDLDSGLNGLIE | | |
| | YSILSGNQEEAFQIDALSGVITTKAILDYELTSSYSLIVQATDKGMPRLSNTTVIKVQ | | |
| | VTDINDNAPAFLPSEAVEITEVMTISEDSLPGVIVTHVSVHDVDLNSAFIFSFAKESN | | |
| | PGTKFAIDQNTGVVVLVKTLDFEEMTEYELLIQISDSVHYTEGALVVRVLDVNDNPPV | | |
| | FSQDFYQVTVPESIPVGYSVLTLSATDLESNENISYRILSSSKEFSIDPKNGTIFTIS | | |
| | PVLLLDTISTTRFLVEASDGGNPDPRALTLVEIGIEDMNNYAPEFTVKSYNLSLSEDA | | |
| | LVGSTLVTFSNIDHDWTRENTYVEYSIISGNSQNNFHVETKFFHSEYPYKQVGYLVLL | | |
| | HSLDREASASHELVILASDSGCPPLSSTAVISIQVLDVNDNPPNFSSLSYHTHVKEST | | |
| | PLGSHITVVSANDRDTGSHAEIIYNIISGNEKGHFYLEENTGVLYLIKPLDYEKMTKF | | |
| | TLTVQASDAEKKHFSFAVVFVSVLDDNDHAPQFMFSSFSCIVPENLPISSTICSINAL | | |
| | DFDAGPYGELTYSIVSPCFLTHGMSYDHDLFLIDPLTGDIHAKQILDYENGNKYCLTV | | |
| | QAKDKGDATASLVVNVDIEGIDEFEPIFTQDQYFFTLPEKNKDRQLIGRVEASDADAG | | |
| | IDGVILYSLGTSSPFFSVNRTNGNIYLIRALPLIKSQLNKEDTLEMKIIAHSPKSDSK | | |
| | FASCTVFVNVSFSSEGTPLAVFASSFSISLVVSFLVFLILICILIVMILRHKQKDTIN | | |
| | NYEEKKTSSLDADLRVTRDASVLKAFQKTDDCSNEVVPVDATPEWLSLISIMEKDIVN | | |
| | LYRYSNSSGHCSVEGETAEDKEIQRINEHPYRKCSDSALSDHESRVPDSGIPRDSDQL | | |
| | SCLSGETDVMVTAETAEASQTFGEGDQGEGCSTTCAQNNVLPQTVQKREAKESILADV | | |
| | RKESVFISGDQEVRCAALSTQTTSDHDGKDNYHWNYLLSWEPKFQPLASVFNDIAKLK | | |
| | DEHLHMPGIPKEKKSFVFPPPLITAVAQPGIKAVPPRMPAVNLGQVPPKHPRSPIPYH | | |
| | LGSLPEGMTPNFSPSLSLLTMQPPALSPLLREGELLGTHISGTCHELKAEDEVQI | | |

Sequence comparison of the above protein sequences yields the following sequence relationships shown in Table 28B.

TABLE 28B
Comparison of NOV28a against NOV28b through NOV28c.

| | | Identities/ |
|---|---|---|
| | NOV28a Residues/ | Similarities |
| Protein Sequence | Match Residues | for the Matched Region |
| NOV28b | 1 . . . 1063 | 1037/1063 (97%) |
| | 1941 . . . 3003 | 1037/1063 (97%) |
| NOV28c | 1 . . . 1063 | 1037/1063 (97%) |
| | 2299 . . . 3361 | 1037/1063 (97%) |

Further analysis of the NOV28a protein yielded the following properties shown in Table 28C.

TABLE 28C
Protein Sequence Properties NOV28a

| | |
|---|---|
| PSort: | 0.8000 probability located in mitochondrial inner membrane; |
| analysis: | 0.7000 probability-located in plasma membrane; |
| | 0.3000 probability located in microbody |
| | (peroxisome); 0.2000 probability located |
| | in endoplasmic reticulum (membrane) |
| SignalP | No Known Signal Sequence Indicated |
| analysis: | |

A search of the NOV28a protein against the Geneseq database, a proprietary database that contains sequences published in patents and patent publication, yielded several homologous proteins shown in Table 28D.

TABLE 28D
Geneseq Results for NOV28a

| | | NOV28a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| AAU68533 | Human novel cytokine encoded by | 1 . . . 646 | 212/680 | (31%) | 2e−76 |
| | cDNA 790CIP2C_4 #1 - | 425 . . . 1072 | 338/680 | (49%) | |
| | Homo sapiens, 3014 aa. | | | | |
| | [WO200175093-A1, | | | | |
| | 11-OCT-2001] | | | | |
| AAE08586 | Human NOV7 protein - Homo sapiens, | 1 . . . 646 | 212/680 | (31%) | 2e−76 |
| | 3028 aa. [WO200161009-A2, | 425 . . . 1072 | 338/680 | (49%) | |
| | 23-AUG-2001] | | | | |
| AAU02196 | Seven-pass transmembrane receptor- | 1 . . . 646 | 212/680 | (31%) | 2e−76 |
| | like protein, MEM1 - Homo sapiens, | 425 . . . 1072 | 338/680 | (49%) | |
| | 3014 aa. [WO200144473-A2, | | | | |
| | 21-JUN-2001] | | | | |
| AAU07054 | Human Flamingo protein encoded by | 1 . . . 611 | 198/627 | (31%) | 1e−73 |
| | 2923 aa. [WO20016003-A1, | 365 . . . 960 | 312/627 | (49%) | |
| | 23-AUG-2001] | | | | |
| AAU07053 | Human Flamingo polypeptide - Homo | 1 . . . 611 | 198/627 | (31%) | 1e−73 |
| | sapiens, 2956 aa. [WO200161003-A1, | 365 . . . 960 | 312/627 | (49%) | |
| | 23-AUG-2001] | | | | |

In a BLAST search of public sequence databases, the NOV28a protein was found to have homology to the proteins shown in the BLASTP data in Table 28E.

TABLE 28E
Public BLASTP Results for NOV28a

| | | NOV28a | Identities | | |
|---|---|---|---|---|---|
| Protein | | Residues/ | Similarities for | | |
| Accession | | Match | the Matched | | Expect |
| Number | Protein/Organism/Length | Residues | Portion | | Value |
| Q9NXU8 | CDNA FLJ20047 FIS, CLONE | 105 . . . 1063 | 959/959 | (100%) | 0.0 |
| | COL00577 - Homo sapiens (Human), | 1 . . . 959 | 959/959 | (100%) | |
| | 959 aa. | | | | |
| Q9NRU0 | CADHERIN-LIKE PROTEIN VR8 - | 189 . . . 582 | 391/394 | (99%) | 0.0 |
| | Homo sapiens (Human), 398 aa | 5 . . . 398 | 393/394 | (99%) | |
| | (fragment). | | | | |
| Q96JQ0 | KIAA1773 PROTEIN - Homo | 1 . . . 1036 | 366/1073 | (34%) | e−148 |
| | sapiens (Human), 3298 aa. | 2245 . . . 3270 | 543/1073 | (50%) | |
| Q24292 | DACHSOUS PROTEIN | 1 . . . 1037 | 327/1165 | (28%) | e−100 |
| | PRECURSOR (ADHERIN) - | 2343 . . . 3471 | 528/1165 | (45%) | |
| | Drosophila melanogaster (Fruit fly), | | | | |
| | 3503 aa. | | | | |
| Q9Y526 | DJ439F8.2 (SUPPORTED BY | 1 . . . 646 | 212/680 | (31%) | 7e−76 |
| | GENSCAN AND GENEWISE) - | 425 . . . 1072 | 338/680 | (49%) | |
| | Homo sapiens (Human), 1181 aa | | | | |
| | (fragment). | | | | |

PFam analysis indicates that the NOV28a protein contains the domains shown in the Table 28F.

TABLE 28F
Domain Analysis of NOV28a

| | NOV28a | Identities/ | |
|---|---|---|---|
| | Match | Similarities for | Expect |
| Pfam Domain | Region | the Matched Region | Value |
| cadherin: domain 1 of 6 | 43 . . . 130 | 26/108 (24%) | 4.5e−15 |
| | | 63/108 (58%) | |
| cadherin: domain 2 of 6 | 144 . . . 232 | 36/108 (33%) | 3.4e−12 |
| | | 65/108 (60%) | |
| cadherin: domain 3 of 6 | 246 . . . 348 | 35/113 (31%) | 5.1e−15 |
| | | 79/113 (70%) | |
| cadherin: domain 4 of 6 | 362 . . . 452 | 34/107 (32%) | 4e−23 |
| | | 73/107 (68%) | |
| cadherin: domain 5 of 6 | 466 . . . 563 | 33/115 (29%) | 1.3e−11 |
| | | 66/115 (57%) | |
| cadherin: domain 6 of 6 | 577 . . . 671 | 27/110 (25%) | 0.11 |
| | | 60/110 (55%) | |

### Example 29

The NOV29 clone was analyzed, and the nucleotide and polypeptide sequences are shown in Table 29A.

TABLE 29A
NOV29 Sequence Analysis

| | |
|---|---|
| SEQ ID NO: 129 | 924 bp |

| | |
|---|---|
| NOV29a, | ATGGGGGAGCAGAACCACTCTCCCGGGAAGGAGCTTCAGCACAGGACGCGAGCAGAGG |
| CG59243-01 DNA | CTCCAGGAAAGAAAAGCTGGCATTCCCAGGCCTATGCCCTTGGGGCCGTTTCCAACTT |
| Sequence | TATGTCTACTTTTCTGACCTTTCCTATCTATAAGGTTGTGTTCCGGCAACAGATCCAT |
| | GCCATGGCAGTGTCAGAGGCTGTGAGACAGCTTTGGCATGAAGGTCCTCAATACTTCT |
| | ACCGGGGAATCTACCCTCCTCTTCTCTCCAAGACGTTGCAAGGGACTCTTCTGTTTGG |
| | GACTTATGATAGCCTGCTGTGCTTTCTCTCTCCTGTTGGGCCACACACCCTGGGACAC |
| | CGCTGGGCTGCCGGGCTCATGTCTGGCGTGGTGGAGGCCGTGGCACTCACCCCCTTTG |
| | AAAGGGTGCAAAATGTGCTCCAGGATGGTCGCAAGCAAGCTCGCTTCCCCAGCACCTT |
| | CAGCATTCTCAAGGAATTCAATTCTTATGGGCTTTGGGGGCGGCTGTCACTGGGCTAC |
| | TATCGTGGTTTCTGGCCTGTCCTGGCCAGGAACAGCCTGGGGAGTGCTCTATATTTTT |
| | CTTTCAAGGACCCCATCCAGGATGGCCTGGCAGAGCAAGGCCTGCCCCACTGGGTTCC |
| | TGCCTTGGTGTCTGGTAGTGTCAATGGAACAATCACCTGCCTAGTTCTGTATCCTCTG |
| | ATTGTGCTGGTTGCTAATATGCAGTCCCATATTGGATGGCAGAACATGCCAAGCCTGT |
| | GGCCCTCTGCCCAGGATGTATGGAACACTCGGGGCCGAAAGCTGCTCCTGATCTACCG |
| | TGGAGGCTCCCTAGTCATCCTAAGGTCCAGTGTGACATGGGGCCTCACTACGGCAATC |
| | CATGACTTCCTGCAGACGAAGTCGCACTCCAGGAAAGAGCTCAAGACTGACTAG |

| ORF Start: ATG at 1 | ORF Stop: TAG at 922 | |
|---|---|---|
| SEQ ID NO: 130 | 307 aa | MW AT 34480.4 kD |

| | |
|---|---|
| NOV29a, | MGEQNHSPGKELQHRTRAEAPGKKSWHSQAYALGAVSNFMSTFLTFPIYKVVFRQQIH |
| CG59243-01 Protein | AMAVSEAVRQLWHEGPQYFYRGIYPPLLSKTLQGTLLFGTYDSLLCFLSPVGPHTLGH |
| Sequence | RWAAGLMSGVVEAVALSPFERVQNVLQDGRKQARFPSTFSILKEFNSYGLWGRLSLGY |
| | YRGFWPVLARNSLGSALYFSFKDPIQDGLAEQCLPHWVPALVSGSVNGTITCLVLYPL |
| | IVLVANNQSHIGWQNNPSLWASAQDVWNTRGRKLLLIYRGCSLVILRSSVTWGLTTAI |
| | HDFLQRKSHSRKELKTD |

| | |
|---|---|
| SEQ ID NO: 131 | 669 bp |

| | |
|---|---|
| NOV29b, | GGATCCATGGGGGAGCAGAACCACTCTCCCGGGAAGGAGCTTCAGCACAGGACGCGAG |
| 188822080 DNA | CAGAGGCTCCAGGAAAGAAAAGCTGGCATTCCCAGGCCCATGCCCTTGGGGCCGTTTC |
| Sequence | CAACTTTATGTCTACTTTTCTGACCTTTCCTATCTATAAGGTTGTGTTCCGGCAACAG |
| | ATCCATGCCATGGCAGTGTCAGAGGCTGTGAGACAGCTTTGGCATGAAGGTCCTCAAT |
| | ACTTCTACCGGGGAATCTACCCTCCTCTTCTCTCCAAGACGTTGCAAGGGACTCTTCT |
| | GTTTGCGACTTATGATAGCCTGCTGTGCTTTCTCTCTCCTGTTGGGCCACACACCCTG |
| | GGACACCGCTGGGCTGCCGGGCTCATGTCTGGCGTGGTGGAGGCCGTGGCACTCAGCC |
| | CCTTTGAAAGGGTGCAAAATGTGCTCCAGGATGGTCGCAAGCAAGCTCGCTTCCCCAG |
| | CACCTTCAGCATTCTCAAGGAATTCAATTCTTATGGGCTTTGGGGGCGGCTGTCACTG |
| | GGCTACTATCGTGGTTTCTGGCCTGTCCTGGCCAGGAACAGCCTGGGGAGTGCTCTAT |
| | ATTTTTCTTTCAAGGACCCCATCCAGGATGGCCTGGCAGAGCAAGGCCTGCCCCACTG |
| | GGTTCCTGCCTTGGTGTCTGGTAGTCTCGAG |

| ORF Start: GGA at 1 | ORF Stop: at 670 | |
|---|---|---|
| SEQ ID NO: 132 | 223 aa | MW at 24883.2 kD |

| | |
|---|---|
| NOV29b, | GSMGEQNHSPGKELQHRTRAEAPGKKSWHSQAHALGAVSNFMSTFLTFPIYKVVFRQQ |
| 188822080 Protein | IHAMAVSEAVRQLWHEGPQYFYRGIYPPLLSKTLQGTLLFGTYDSLLCFLSPVGPHTL |
| Sequence | CHRWAAGLMSGVVEAVALSPFERVQNVLQDGRKQARFPSTFSILKEFNSYGLWGRLSL |
| | GYYRGFWPVLARNSLGSALYFSFKDPIQDGLAEQGLPHWVPALVSGSLE |

| | |
|---|---|
| SEQ ID NO: 133 | 924 bp |

| | |
|---|---|
| NOV29c, | ATGGGGGAGCAGAACCACTCTCCCGGGAAGGAGCTTCAGCACAGGACGCCAGCAGAGG |
| CG59243-02 DNA | CTCCAGGAAAGAAAAGCTGGCATTCCCAGGCCTATGCCCTTGGGGCCGTTTCCAACTT |
| Sequence | TATGTCTACTTTTCTGACCTTTCCTATCTATAAGCTTGTGTTCCGGCAACAGATCCAT |
| | GCCATGGCAGTGTCACAGGCTGTCAGACAGCTTTGGCATGAAGGTCCTCAATACTTCT |
| | ACCGGGGAATCTACCCTCCTCTTCTCTCCAAGACGTTGCAAGGGACTCTTCTGTTTGG |
| | GACTTATGATAGCCTGCTGTGCTTTCTCTCTCCTGTTGGGCCACACACCCTGGGACAC |
| | CGCTGGGCTCCCGGGCTCATGTCTGGCGTGGTGGAGGCCGTGGCACTCAGCCCCTTTG |
| | AAAGGGTGCAAAATGTGCTCCAGGATGGTCGCAAGCAAGCTCGCTTCCCCAGCACCTT |
| | CACCATTCTCAAGGAATTCAATTCTTATGGGCTTTGGGGGCGGCTGTCACTGGGCTAC |
| | TATCGTGGTTTCTGGCCTGTCCTGGCCAGGAACAGCCTGGGCAGTGCTCTATATTTTT |
| | CTTTCAAGGACCCCATCCAGGATGGCCTGGCAGAGCAAGGCCTGCCCCACTGGGTTCC |
| | TGCCTTGGTGTCTGGTAGTGTCAATGGAACAATCACCTGCCTAGTTCTGTATCCTCTG |
| | ATTGTGCTGGTTGCTAATATGCAGTCCCATATTGGATGGCAGAACATGCCAAGCCTGT |
| | GGGCCTCTGCCCAGGATGTATGGAACACTCGGGGCCGAAAGCTGCTCCTGATCTACCG |
| | TGGAGGCTCCCTAGTCATCCTAAGGTCCAGTGTGACATGGGGCCTCACTACGGCAATC |
| | CATGACTTCCTGCAGAGGAAGTCGCACTCCAGGAAAGAGCTGAAGACTGACTAG |

| ORF Start: ATG at 1 | ORF Stop: TAG at 922 | |
|---|---|---|
| SEQ ID NO: 134 | 307 aa | MW at 34480.4 kD |

| | |
|---|---|
| NOV29c, | MGEQNHSPGKELQHRTRAEAPGKKSWHSQAYALGAVSNFMSTFLTFPIYKVVFRQQIH |
| CG59243-02 Protein | AMAVSEAVRQLWHEGPQYFYRGIYPPLLSKTLQGTLLFGTYDSLLCFLSPVGPHTLGH |
| Sequence | RWAAGLMSGVVEAVALSPFERVQNVLQDGRKQARFPSTFSILKEFNSYGLWGRLSLGY |
| | YRGFWPVLARNSLGSALYFSFKDPIQDGLAEQGLPHWVPALVSGSVNGTITCLVLYPL |
| | IVLVANMQSHIGWQNMPSLWASAQDVWNTRGRKLLLIYRGGSLVILRSSVTWGLTTAI |
| | HDFLQRKSHSRKELKTD |

Sequence comparison of the above protein sequences yields the following sequence relationships shown in Table 29B.

TABLE 29B
Comparison of NOV29a against NOV29b through NOV29c.

| | NOV29a Residues/ | Identities/Similarities |
|---|---|---|
| Protein Sequence | Match Residues | for the Matched Region |
| NOV29b | 3 . . . 222 | 220/220 (99%) |
| NOV29c | 1 . . . 307 | 307/307 (100%) |
| | 1 . . . 307 | 307/307 (100%) |

Further analysis of the NOV29a protein yielded the following properties shown in Table 29C.

TABLE 29C
Protein Sequence Properties NOV29a

| | |
|---|---|
| PSort | 0.7000 probability located in plasma membrane; |
| analysis: | 0.6400 probability located in microbody (peroxisome); |
| | 0.2000 probability located in endoplasmic reticulum |
| | (membrane); 0.1000 probability located in mitochondrial |
| | inner membrane |
| SignalP | No Known Signal Sequence Indicated |
| analysis: | |

A search of the NOV29a protein against the Geneseq database, a proprietary database that contains sequences published in patents and patent publication, yielded several homologous proteins shown in Table 29D.

TABLE 29D
Geneseq Results for NOV29a

| | | NOV29a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| AAM40554 | Human polypeptide SEQ ID NO 5485 - | 31 . . . 294 | 93/269 | (34%) | 3e−39 |
| | Homo sapiens, 304 aa. | 41 . . . 300 | 146/269 | (53%) | |
| | [WO200153312-A1, 26-JUL-2001] | | | | |
| AAM38768 | Human polypeptide SEQ ID NO 1913 - | 31 . . . 294 | 93/269 | (34%) | 3e−39 |
| | Homo sapiens, 297 aa. | 34 . . . 293 | 146/269 | (53%) | |
| | [WO200153312-A1, 26-JUL-2001] | | | | |
| AAB60090 | Human transport protein TPPT-10 - | 31 . . . 294 | 93/269 | (34%) | 3e−39 |
| | Homo sapiens, 297 aa. | 34 . . . 293 | 146/269 | (53%) | |
| | [WO200078953-A2, 28-DEC-2000] | | | | |
| AAY97294 | Lipid associated protein (LIPAP) | 31 . . . 294 | 93/269 | (34%) | 3e−39 |
| | 3735780CD1 - Homo sapiens, 297 aa. | 34 . . . 293 | 146/269 | (53%) | |
| | [WO200049043-A2, 24-AUG-2000] | | | | |
| AAG03236 | Human secreted protein, SEQ ID NO: | 46 . . . 106 | 26/61 | (42%) | 4e−08 |
| | 7317 - Homo sapiens, 95 aa. | 19 . . . 79 | 38/61 | (61%) | |
| | [EP1033401-A2, 06-SEP-2000] | | | | |

In a BLAST search of public sequence databases, the NOV29a protein was found to have homology to the proteins shown in the BLASTP data in Table 29E.

TABLE 29E
Public BLASTP Results for NOV29a

| | | NOV29a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9H1U9 | BA3J10.3 (CG7943 PROTEIN) | 31 . . . 294 | 93/269 (34%) | 1e−38 |
| | (UNKNOWN) (PROTEIN FOR | 34 . . . 293 | 146/269 (53%) | |
| | MGC: 14836) - Homo sapiens | | | |
| | (Human), 297 aa. | | | |
| Q960B8 | SD08549P -Drosophila melanogaster | 25 . . . 296 | 83/275 (30%) | 2e−31 |
| | (Fruit fly), 332 aa. | 51 . . . 319 | 146/275 (52%) | |
| Q9VAB1 | CG7943 PROTEIN - Drosophila | 25 . . . 296 | 83/275 (30%) | 2e−31 |
| | melanogaster (Fruit fly), 349 aa. | 68 . . . 336 | 146/275 (52%) | |
| O01256 | T20D3.5 PROTEIN - Caenorhabditis | 88 . . . 296 | 71/221 (32%) | 3e−19 |
| | elegans, 221 aa. | 2 . . . 215 | 103/221 (46%) | |
| Q9V3T2 | CG5254 PROTEIN - Drosophila | 56 . . . 240 | 55/195 (28%) | 5e−11 |
| | melanogaster (Fruit fly), 306 aa. | 57 . . . 242 | 99/195 (50%) | |

PFam analysis indicates that the NOV29a protein contains the domains shown in the Table 29F.

TABLE 29F
Domain Analysis of NOV29a

| | NOV29a | Identities/ | |
|---|---|---|---|
| | Match | Similarities for | Expect |
| Pfam Domain | Region | the Matched Region | Value |
| mito_carr: domain 1 of 3 | 26 . . . 112 | 19/125 (15%) | 0.0057 |
| | | 62/125 (50%) | |
| mito_carr: domain 2 of 3 | 113 . . . 209 | 23/130 (18%) | 5.6e−07 |
| | | 70/130 (54%) | |
| mito_carr: domain 3 of 3 | 210 . . . 304 | 18/126 (14%) | 0.037 |
| | | 61/126 (48%) | |

### Example 30

The NOV30 clone was analyzed, and the nucleotide and polypeptide sequences are shown in Table 30A.

TABLE 30A
NOV30 Sequence Analysis

| | |
|---|---|
| SEQ ID NO: 135 | 5101 bp |

| | |
|---|---|
| NOV3Oa, | ATCCTCCTACCTCAGTCTACTGAGTAGCTAGGACTACAGGCATGTGCCACCACACCTG |
| CG59534-01 DNA | GCTAGTTTTTATTTTTTGATGAGATGGGATCTTGCTGTGTTGCCCAGACTGGTCTTGA |
| Sequence | TCTCCTGGGCTCAAGTGATCCTCCTGCCTTCGCCTGCGGCCGCCTGCACTTGCGCTGG |
| | GGACTCGCTGGACTGCGGTGGGCGCCGGCTGGCTGCGTTGCCCGGGGACCTCCCCTCC |
| | TCGACGCGGAGCCTAAACCTGAGTTACAACAAACTCTCTGACATTGACCCTGCTCGTT |
| | TTGAGGACTTGCCGAACCTACAGGAAGTGTACCTCAATAATAATGAGTTGACAGCGGT |
| | ACCATCCCTGGGCGCTGCTTCATCACATGTCGTCTCTCTCTTTCTGCAGCACAACAAG |
| | ATTCGCAGCGTGGAGGGGAGCCAGCTGAAGGCCTACCTTTCCTTAGAAGTGTTAGATC |
| | TGAGTTTGAACAACATCACGGAAGTGCGGAACACCTGCTTTCCACACGGACCGCCTAT |
| | AAAGGAGCTGAACCTGGCAGCCAATCGGATTGGCACCCTGGAGTTGGGAGCATTTGAT |
| | GGTCTGTCACGGTCGCTGCTAACTCTTCGCCTGAGCAAAAACAGGATCACCCAGCTTC |
| | CTGTAAGAGCATTCAAGCTACCCAGGCTGACACAACTGGACCTCAATCGGAACAGGAT |
| | TCGGCTGATAGAGGGCCTCACCTTCCAGGGGCTCAACACCTTGGAGGTGCTGAAGCTT |
| | CAGCGAAACAACATCACCAAACTGACAGATGGGGCCTTCTGGGGACTGTCCAAGATGC |
| | ATGTGCTGCACCTGGAGTACAACAGCCTGGTAGAAGTGAACAGCGGCTCGCTCTACGC |
| | CCTCACGGCCCTGCATCAGCTCCACCTCAGCAACAATTCCATCGCTCGCATTCACCGC |
| | AAGGGCTGGAGCTTCTGCCAGAAGCTGCATGAGTTGGTCCTGTCCTTCAACAACCTGA |
| | CACGGCTGGACGAGGAGAGCCTGGCCGAGCTGAGCAGCCTGAGTGTCCTGCGTCTCAG |
| | CCACAATTCCATCAGCCACATTGCGGAGGGTGCCTTCAAGGCACTCACCACCCTGCGA |
| | GTCTTGGATCTGGACCATAACGAGATTTCGGGCACAATAGAGGACACGAGCGGCGCCT |
| | TCTCAGCGCTCGACAGCCTCAGCAAGCTGACTCTGTTTGGAAACAAGATCAAGTCTGT |
| | GGCTAAGAGAGCATTCTCGGGGCTGGAAGGCCTGGAGCACCTGAACCTTGGAGGGAAT |
| | GCGATCAGATCTGTCCAGTTTGATGCCTTTGTGAAGATGAAGAATCTTAAAGAGCTCC |
| | ATATCAGCAGCGACAGCTTCCTGTGTGACTGCCAGCTGAAGTGGCTGCCCCCGTGGCT |
| | AATTGGCAGGATGCTGCAGGCCTTTGTGACAGCCACCTGTGCCCACCCAGAATCACTG |
| | AAGGGTCAGAGCATTTTCTCTGTGCCACCAGAGAGTTTCGTGTGCGATGACTTCCTGA |
| | AGCCACAGATCATCACCCAGCCAGAAACCACCATGCCTATGGTGGGCAAGGACATCCG |
| | GTTTACATGCTCAGCAGCCAGCAGCAGCAGCTCCCCCATGACCTTTGCCTGGAAGAAA |
| | GACAATGAAGTCCTGACCAATGCAGACATGGAGAACTTTGTCCACGTCCACCCGCAGG |
| | ACGGGGAAGTGATGGAGTACACCACCATCCTGCACCTCCGTCAGGTCACTTTCGGGCA |
| | CGAGGGCCGCTACCAATGTGTCATCACCAACCACTTTGGCTCCACCTATTCACATAAG |
| | GCCAGGCTCACCGTGAATGTGTTGCCATCATTCACCAAAACGCCCCACGACATAACCA |
| | TCCGGACCACCACCATGGCCCGCCTCGAATGTGCTGCCACAGGTCACCCAAACCCTCA |
| | GATTGCCTGGCAGAAGGATGGAGGCACGGATTTCCCCGCTGCCCGTGAGCGACGCATG |
| | CATGTCATGCCGGATGACGACGTGTTTTTCATCACTGATGTGAAAATAGATGACGCAG |
| | GGGTTTACAGCTGTACTGCTCAGAACTCAGCCGGTTCTATTTCAGCTAATGCCACCCT |
| | GACTGTCCTAGAGACCCCATCCTTGGTGGTCCCCTTGGAAGACCGTGTCGTATCTGTG |
| | GGAGAAACAGTGGCCCTCCAATGCAAAGCCACGGGGAACCCTCCGCCCCGCATCACCT |
| | GGTTCAAGGGGGACCGCCCGCTGAGCCTCACTGAGCGGCACCACCTGACCCCTGACAA |
| | CCAGCTCCTGGTGGTTCAGAACGTGGTGGCAGAGGATGCCGGCCGATATACCTGTGAG |
| | ATGTCCAACACCCTGGGCACGGAGCGAGCTCACAGCCAGCTGAGCGTCCTGCCCGCAG |
| | CAGGCTGCAGGAAGGATGGCACCACGGTAGGCATCTTCACCATTGCTGTCGTGAGCAG |
| | CATCGTCCTGACGTCACTGGTCTGCGTGTGCATCATCTACCAGACCAGGAAGAAGAGT |
| | GAAGAGTACAGTGTCACCAACACAGATGAAACCGTCGTGCCACCAGATGTTCCAAGCT |
| | ACCTCTCTTCTCAGGGGACCCTTTCTGACCGACAAGAAACCGTGGTCAGGACCGAGGG |
| | TGGCCCTCAGGCCAATGGGCACATTGAGAGCAATGGTGTGTGTCCAAGAGATGCAAGC |
| | CACTTTCCAGAGCCCGACACTCACAGCGTTGCCTGCAGGCAGCCAAAGCTCTGTGCTG |
| | GGTCTGCGTATCACAAAGAGCCGTGGAAAGCGATGGAGAAAGCTGAACGGACACCTGG |
| | GCCACATAAGATGGAACACGGTGGCCGGGTCGTATGCAGTGACTGCAACACCGAAGTG |
| | GACTGTTACTCCAGGGGACAAGCCTTCCACCCCCAGCCTCTGTCCAGAGACAGCGCAC |
| | AGCCAAGTGCGCCAAATGGCCCGGAGCCGGGTGGGAGTGACCAAGAGCATTCTCCACA |
| | TCACCAGTGCAGCAGGACTGCCGCTGGCTCCTGCCCCGAGTGCCAAGGGTCGCTCTAC |
| | CCCAGTAACCACGATAGAATGCTGACGGCTGTGAAGAAAAAGCCAATGGCATCTCTAG |
| | ATGGGAAAGGGGATTCTTCCTGGACTTTAGCAAGGTTGTATCACCCGGACTCCACAGA |
| | GCTACAGCCTGCATCTTCATTAACTTCAGGCAGTCCAGAGCGCGCGGAAGCCCAGTAC |
| | TTGCTTGTTTCCAATGGCCACCTCCCCAAAGCATGTGACGCCAGTCCCGAGTCCACGC |
| | CACTGACAGGACAGCTCCCCGGGAAACAGAGGGTGCCACTGCTGTTGGCACCAAAAAG |
| | CTAGGTTTTGTCTACCTCAGTTCTTGTCATACCAATCTCTACGGGAAAGAGAGGTAGG |
| | AGAGGCTGCGAGGAAGCTTGGGTTCAAGCGTCACTCATCTGTACATAGTTGTAACTCC |
| | CATGTGGAGTATCAGTCGCTCACAGGACTTGGATCTGAAGCACAGTAAACGCAAGAGG |
| | GGATTTGTGTACAAAAGGCAAAAAAAAGTATTTGATATCATTGTACATAAGAGTTTTC |
| | AGAGATTTCATATATATCTTTTACAGAGGCTATTTTAATCTTTAGTGCATGGTTAACA |
| | GAAAAAAATTATACAATTTTGACAATATTATTTTTCGTATCAGGTTGCTGTTTAATTT |
| | TGGAGGGGGTGGGGAAATAGTTCTGGTGCCTTAACGCATGGCTGGAATTTATAGAGGC |
| | TACAACCACATTTGTTCACAGGAGTTTTTGGTGCGGGGTGGGAAGGATGGAAGGCCTT |
| | GGATTTATATTGCACTTCATAGACCCCTAGGCTGCTGTGCGGTGGGACTCCACATGCG |
| | CCGGAAGGAGCTTCAGGTGAGCACTGCTCATGTGTGGATGCCCCTGCAACAGGCTTCC |
| | CTGTCTGTAGAGCCAGGGGTGCAAGTGCCATCCACACTTGCAGTGAATGGCTTTTCCT |
| | TTTAGGTTTAAGTCCTGTCTGTCTGTAAGGCGTAGAATCTGTCCGTCTGTAAGGCGTA |
| | GAATGAGGGTTGTTAATCCATCACAAGCAAAAGGTCAGAACAGTTAAACACTGCCTTT |
| | CCTCCTCCTCTTATTTTATGATAAAAGCAAATGTGGCCTTCTCAGTATCATTCGATTG |
| | CTATTTGAGACTTTTAAATTAAGGTAAAGGCTGCTGGTGTTGGTACCTGTGGATTTTT |
| | CTATACTGATGTTTTCGTTTTGCCAATATAATGAGTATTACATTGGCCTTGGGGGACA |
| | GAAAGGAGGAAGTTCTGACTTTTCAGGGCTACCTTATTTCTACTAAGGACCCAGAGCA |
| | GGCCTGTCCATGCCATTCCTTCGCACAGATGAAACTGAGCTCCCACTCGAAAGGACAG |
| | CCCTTGACCTGGGTTCTGGGTATAATTTGCACTTTTGAGACTGGTAGCTAACCATCTT |
| | ATGAGTGCCAATGTGTCATTTAGTAAAACTTAAATAGAAACAAGGTCCTTCAAATGTT |
| | CCTTTGGCCAAAAGCTGAAGGGAGTTACTGAGAAAATAGTTAACAATTACTGTCAGGT |
| | GTCATCACTGTTCAAAAGGTAAGCACATTTAGAATTTTGTTCTTGACAGTTAACTGAC |
| | TAATCTTACTTCCACAAAATATGTGAATTTGCTGCTTCTGAGAGGCAATGTGAAAGAG |
| | GGAGTATTACTTTTATGTACAAAGTTATTTATTTATAGAAATTTTGGTACAGTGTACA |
| | TTGAAAACCATGTAAAATATTGAAGTGTCTAACAAATGGCATTGAAGTGTCTTTAATA |
| | AAGGTTCATTTATAAATGTCAGTATAGTTGGTGGTCCTTCTTTTACAAACGCAGTCAT |
| | TCTGCCTTTAATTATCTTCCCCCAAAAAAGAAAAAAAAAATAGGCGAAGCAAAATCAC |
| | ATACTGTTTGTTTGCTCCAGGGCAGACAACACTGCTAGATTCCTCACATTTTGTTTTG |
| | AATTTTTCTACACCTGGAGCTTGTTAGTCAAGGTCTAAAATCCCTAAGTGTGGTGACC |
| | TTTCCATTTCATCCTGCCTTTTCAAAGCTGGCCCAGGCCCTCCTTTCAGTCTGACATG |
| | AGAATGGCGAGAATGGCTCACCCACCGTGCCCTCCTGCACGAAGCCAGCTGGGCC |

| ORF Start: ATG at 77 | ORF Stop: TAG at 3308 | |
|---|---|---|
| SEQ ID NO: 136 | 1077 aa | MW at 117735.2 kD |

| | |
|---|---|
| NOV3Oa, | MRWDLAVLPRLVLISWAQVILLPWPAAACTCAGDSLDCGGRGLAALPCDLPSWTRSLN |
| CG59534-01 Protein | LSYNKLSEIDPAGFEDLPNLQEVYLNNNELTAVPSLGAASSHVVSLFLQHNKIRSVEG |
| Sequence | SQLKAYLSLEVLDLSLNNITEVRNTCFPHGPPIKELNLAGNRIGTLELGAFDGLSRSL |
| | LTLRLSKNRITQLPVRAFKLPRLTQLDLNRNRIRLIEGLTFQGLNSLEVLKLQRNNIS |
| | KLTDGAFWGLSKMHVLHLEYNSLVEVNSGSLYGLTALHQLHLSNNSIARIHRKGWSFC |
| | QKLHELVLSFNNLTRLDEESLAELSSLSVLRLSHNSISHIAEGAFKGLRSLRVLDLDH |
| | NEISGTIEDTSGAFSGLDSLSKLTLFGNKIKSVAKRAFSGLEGLEHLNLGGNAIRSVQ |
| | FDAFVKMKNLKELHISSDSFLCDCQLKWLPPWLIGRMLQAFVTATCAHPESLKGQSIF |
| | SVPPESFVCDDFLKPQIITQPETTMAMVGKDIRFTCSAASSSSSPMTFAWKKDNEVLT |
| | NADMENFVHVHAQDGEVMEYTTILHLRQVTFGHEGRYQCVITNHFGSTYSHKARLTVN |
| | VLPSFTKTPHDITIRTTTMARLECAATGHPNPQIAWQKDGGTDFPPARERRMHVMPDD |
| | DVFFITDVKIDDACVYSCTAQNSAGSISANATLTVLETPSLVVPLEDRVVSVGETVAL |
| | QCKATGNPPPRITWFKGDRPLSLTERHHLTPDNQLLVVQNVVAEDAGRYTCEMSNTLG |
| | TERHSQLSVLPAAGCRKDGTTVGIFTIAVXJSSIVLTSLVWVCIIYQTRKKSEEYSVT |
| | NTDETVVPPDVPSYLSSQGTLSDRQETVVRTEGGPQANGHIESNGVCPRDASHFPEPD |
| | THSVACRQPKLCAGSAYHKEPWKAMEKAEGTPGPHKMEHGGRVVCSDCNTEVDCYSRG |
| | QAFHPQPVSRDSAQPSAPNGPEPGGSDQEHSPHHQCSRTAAGSCPECQGSLYPSNHDR |
| | MLTAVKKKPMASLDGKGDSSWTLARLYHPDSTELQPASSLTSGSPERAEAQYLLVSNG |
| | HLPKACDASPESTPLTGQLPGKQRVPLLLAPKS |

Further analysis of the NOV30a protein yielded the following properties shown in Table 30B.

TABLE 30B
Protein Sequence Properties NOV30a

| | |
|---|---|
| PSort | 0.4600 probability located in plasma membrane; 0.1000 |
| analysis: | probability located in endoplasmic reticulum (membrane); |
| | 0.1000 probability located in endoplasmic reticulum (lumen); |
| | 0.1000 probability located in outside |
| SignalP | Cleavage site between residues 29 and 30 |
| analysis: | |

A search of the NOV30a protein against the Geneseq database, a proprietary database that contains sequences published in patents and patent publication, yielded several homologous proteins shown in Table 30C.

TABLE 30C
Geneseq Results for NOV30a

| | | | | |
|---|---|---|---|---|
| | | NOV30a | | |
| | | Residues/ | Identities/ | |
| Geneseq | Protein/Organism/Length [Patent #, | Match | Similarities for the | Expect |
| Identifier | Date] | Residues | Matched Region | Value |

| | | | | |
|---|---|---|---|---|
| AAY08008 | Human HLIG-1 protein - Homo sapiens, | 29 . . . 1077 | 1030/1050 (98%) | 0.0 |
| | 1101 aa. [EP913472-A2, | 52 . . . 1101 | 1040/1050 (98%) | |
| | 06-MAY-1999] | | | |
| AAY97833 | Murine LIG-1 protein - Mus sp, 1091 | 9 . . . 1077 | 890/1088 (81%) | 0.0 |
| | aa. [JP2000093043-A, 04-APR-2000] | 16 . . . 1091 | 950/1088 (86%) | |
| AAY08010 | Mouse LIG-1 protein - Mus sp, 1091 | 9 . . . 1077 | 890/1088 (81%) | 0.0 |
| | aa. [EP913472-A2, 06-MAY-1999] | 16 . . . 1091 | 950/1088 (86%) | |
| AAY08099 | Murine glial cell membrane | 9 . . . 1077 | 890/1088 (81%) | 0.0 |
| | glycoprotein LIG-1 - Mus sp, 1091 | 16 . . . 1091 | 950/1088 (86%) | |
| | aa. [WO9914241-A2, 25-MAR-1999] | | | |
| AAW41641 | Sequence used in detection method - | 9 . . . 1077 | 890/1088 (81%) | 0.0 |
| | Mus sp, 1091 aa. [JP09107971-A, | 16 . . . 1091 | 950/1088 (86%) | |
| | 28-APR-1997] | | | |

In a BLAST search of public sequence databases, the NOV30a protein was found to have homology to the proteins shown in the BLASTP data in Table 30D.

TABLE 30D
Public BLASTP Results for NOV30a

| | | NOV30a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q96JA1 | MEMBRANE GLYCOPROTEIN | 26 . . . 1077 | 1051/1052 (99%) | 0.0 |
| | LIG-1 - Homo sapiens (Human), | 42 . . . 1093 | 1052/1052 (99%) | |
| | 1093 aa. | | | |
| Q9BYB8 | MEMBRANE GLYCOPROTEIN | 26 . . . 1077 | 1051/1053 (99%) | 0.0 |
| | LIG-1 - Homo sapiens (Human), | 42 . . . 1094 | 1052/1053 (99%) | |
| | 1094 aa. | | | |
| P70193 | MEMBRANE GLYCOPROTEIN - | 9 . . . 1077 | 890/1088 (81%) | 0.0 |
| | Mus musculus (Mouse), 1091 aa. | 16 . . . 1091 | 950/1088 (86%) | |
| Q9UFI4 | HYPOTHETICAL 51.9 KDA | 595 . . . 1077 | 480/483 (99%) | 0.0 |
| | PROTEIN - Homo sapiens | 1 . . . 483 | 481/483 (99%) | |
| | (Human), 483 aa (fragment). | | | |
| O94898 | KIAA0806 PROTEIN - Homo | 7 . . . 1002 | 507/1026 (49%) | 0.0 |
| | sapiens (Human), 1065 aa. | 20 . . . 991 | 648/1026 (62%) | |

PFam analysis indicates that the NOV30a protein contains the domains shown in the Table 30E.

TABLE 30E
Domain Analysis of NOV30a

| | | Identities/ | |
|---|---|---|---|
| | | Similarities | |
| | NOV30a Match | for the Matched | Expect |
| Pfam Domain | Region | Region | Value |
| LRRNT: domain 1 of 1 | 26 . . . 51 | 14/31 (45%) | 0.34 |
| | | 19/31 (61%) | |
| LRR: domain 1 of 15 | 53 . . . 76 | 10/25 (40%) | 0.88 |
| | | 19/25 (76%) | |
| LRR: domain 2 of 15 | 77 . . . 98 | 9/25 (36%) | 0.71 |
| | | 17/25 (68%) | |
| LRR: domain 3 of 15 | 100 . . . 120 | 5/25 (20%) | 1.1e+02 |
| | | 17/25 (68%) | |
| LRR: domain 4 of 15 | 124 . . . 147 | 10/25 (40%) | 4.4 |
| | | 18/25 (72%) | |
| LRR: domain 5 of 15 | 148 . . . 171 | 7/25 (28%) | 3.9 |
| | | 18/25 (72%) | |
| LRR: domain 6 of 15 | 173 . . . 195 | 10/25 (40%) | 0.22 |
| | | 18/25 (72%) | |
| LRR: domain 7 of 15 | 196 . . . 219 | 7/25 (28%) | 0.6 |
| | | 19/25 (76%) | |
| LRR: domain 8 of 15 | 220 . . . 243 | 21/25 (84%) | 0.0044 |
| LRR: domain 9 of 15 | 244 . . . 267 | 6/25 (24%) | 1.8 |
| | | 18/25 (72%) | |
| LRR: domain 10 of 15 | 268 . . . 291 | 6/25 (24%) | 34 |
| | | 14/25 (56%) | |
| LRR: domain 11 of 15 | 292 . . . 315 | 12/25 (48%) | 0.01 |
| | | 19/25 (76%) | |
| LRR: domain 12 of 15 | 316 . . . 339 | 8/25 (32%) | 0.00037 |
| | | 21/25 (84%) | |
| IGPD: domain 1 of 1 | 329 . . . 342 | 10/15 (67%) | 1.1 |
| | | 12/15 (80%) | |
| LRR: domain 13 of 15 | 340 . . . 363 | 8/25 (32%) | 0.1 |
| | | 20/25 (80%) | |
| LRR: domain 14 of 15 | 367 . . . 390 | 7/25 (28%) | 2.5 |
| | | 16/25 (64%) | |
| LRR: domain 15 of 15 | 391 . . . 414 | 7/25 (28%) | 0.49 |
| | | 18/25 (72%) | |
| LRRCT: domain 1 of 1 | 424 . . . 474 | 22/54 (41%) | 2.1e−13 |
| | | 43/54 (80%) | |
| ig: domain 1 of 3 | 493 . . . 563 | 12/72 (17%) | 1.4e−06 |
| | | 49/72 (68%) | |
| ig: domain 2 of 3 | 597 . . . 658 | 13/65 (20%) | 1.3e−07 |
| | | 46/65 (71%) | |
| ig: domain 3 of 3 | 691 . . . 749 | 18/62 (29%) | 2.9e−11 |
| | | 47/62 (76%) | |
| Adeno_E3_CR1: | 687 . . . 764 | 23/89 (26%) | 1.3 |
| domain 1 of 1 | | 45/89 (51%) | |

### Example 31

The NOV31 clone was analyzed, and the nucleotide and polypeptide sequences are shown in Table 31A.

TABLE 31A
NOV31 Sequence Analysis

| | SEQ ID NO:137 | 4200 bp |
|---|---|---|
| NOV31a, | TAACGAGGTCTAATTTAATTCTCCCAACACCCTATGAAGTCAGCTCTGATACCCCTAG | |
| CG59289-01 DNA | TTTACAGAGGAGCTAAACGAGGCCCAGAGATGGCCAGTCCCAGAGCCAGGAAGCCCCA | |
| Sequence | GAGCCAGTGCTGGGATTTCACAGTACACTGTGCTACTTCCCTAGGCAAAGTCACGTCT | |
| | CTTCTCTGTCTCTACAAAACGAGGACAGACTGGCTTACTCCGAGCGCTGGTATAAGGA | |
| | TTGGAAGAGAATGCAGTGAGCGGTTCCCCACCCCAGGAGGTAACTCCCTGGAACTGTG | |
| | CTCTGAGCCCAAACTCTCAAGGGTTGGTCAGTGCCAGGCACAGGGTAGGTGCTCCGTC | |
| | CCTGACAGCTATTGTTGCAATCGAGGTAACTCCCTGGAACTGTGCTCTGAGCCCPAAC | |
| | TCTCAAGGGTTCGTCAGTGCCACCCACAGGGGACGCTGCCTTCAGAGCCCCCCAGTGC | |
| | CTGTGCCTCAGACCCGTGCGCTCCAGGGACCGAGTGCCAGGCTACCGAGAGTGGTGGC | |
| | TATACCTGTGGGCCCATGGAGCCCCGGGGCTGTGCCACCCAGCCATGCCACCACGGCG | |
| | CTCTGTGTGTGCCCCAGGGTCCAGATCCCACCGGCTTCCGCTGCTACTCCGTGCCGGG | |
| | TTTCCAGGGCCCACGCTGCGAGCTGGACATCGATGAGTGTGCATCCCGGCCGTGCCAC | |
| | CATGGGGCCACCTGCCGCAACCTGGCCGATCGCTACGAGTGCCATTGCCCCCTTGGCT | |
| | ATGCAGGCGTGACCTGCGAGATGGAGGTGGACGAGTGCGCCTCAGCGCCCTGCCTOCA | |
| | CCGGGGCTCGTGCCTGGACGGCGTGGGCTCCTTCCGCTCTGTGTGCGCGCCAGGCTAC | |
| | GGGGGCACCCGTTGCCAGCTGGACCTCGACGAGTGCCAGAGCCAGCCGTGCGCACATG | |
| | GGGGCACGTGCCACGACCTCGTCAACGGGTTCCGGTGCGACTGCGCGGGCACCGGCTA | |
| | CGAGGGCACGCACTGCGAGCGGGAGGTGCTGGAGTGCCCATCGGCGCCCTGCGAGCAC | |
| | AACGCGTCCTGCCTCGAGGGCCTCGGGAGCTTCCGCTGCCTCTGTTGGCCAGGCTACA | |
| | GCGGCCAGCTGTGCGAGGTGGACGAGGACGAGTGTGCATCGAGCCCCTGCCAGCATGG | |
| | GGGCCGATGCCTGCAGCGCTCTGACCCGGCCCTCTACGGGGGTGTCCAGGCCGCCTTC | |
| | CCTGGCGCCTTCAGCTTCCGCCATGCTGCGGGTTTCCTGTGCCACTGCCCTCCTGGCT | |
| | TTGAGGGAGCCGACTGCGGTGTGGAGGTGGACGAGTGTGCCTCACGGCCATGCCTCAA | |
| | CCCAGGCCACTGCCAGGACCTGCCCAATGGCTTCCAGTGTCACTGCCCAGATGGCTAC | |
| | GCAGGGCCGACATGTGAGGAAGATGTGGATGAATGCCTGTCGGATCCCTGCCTGCACG | |
| | GCGGAACCTGCAGTGACACTGTGGCAGGCTATATCTGCAGGTGCCCAGAGACCTGGGG | |
| | TGGGCGCGACTGTTCTGTGCAGCTCACTGGCTGCCAGGGCCACACCTGCCCGCTGGCT | |
| | GCCACCTGCATCCCTATCTTCGAGTCTGGGGTCCACAGTTACGTCTGCCACTGCCCAC | |
| | CTGGTACCCATGGACCGTTCTGTGGCCAGAATACCACCTTCTCTGTGATGGCTGCGAG | |
| | CCCCATTCAGGCATCAGTGCCAGCTGGTGGCCCCCTGGGTCTGGCACTGAGGTTTCGC | |
| | ACCACACTGCCCGCTGGGACCTTGGCCACTCGCAATGACACCAAGGAAAGCTTGGAGC | |
| | TGGCATTGGTGGCAGCCACACTTCAGGCCACACTCTGGAGCTACAGCACCACTGTGCT | |
| | TGTCCTGAGACTGCCGGACCTGGCCCTAAACCATGGCCATTCGCACCAGGTGGAGGTT | |
| | GTGCTCCATCTAGCGACCCTGGAGCTACGGCTCTGGCATGAGGGCTGCCCTGCCCGGC | |
| | TCTGTGTGGCCTCTGGTCCTGTCGCCCTGCCTTCCACGGCTTCGGCAACTCCGCTGCC | |
| | TGCCGGCATCTCCTCTGCCCAGCTGGGGGACGCGACCTTTGCAGGCTGCCTCCAGGAC | |
| | GTGCGTGTGGATGGCCACCTCCTGCTGCCTGAGGATCTCGGTGAGAACGTCCTCCTGG | |
| | GCTGTGAGCGCCGAGAGCAGTGCCGGCCTCTGCCTTGTGTCCACGGAGGGTCCTGTGT | |
| | GGATCTGTGGACTCATTTCCGTTGCGACTGTGCCCGGCCCCATAGAGGTCCCACGTGC | |
| | GCTGATGAGATTCCTGCTGCCACCTTTGGCTTGGGAGGCGCCCCAAGCTCTGCCTCCT | |
| | TTCTGCTCCAAGAGCTGCCAGGTCCCAACCTCACACTGTCTTTCCTTCTCCCCACTCG | |
| | GGAGTCCGCTGGCCTGTTGCTCCAGTTTGCCAATGACTCCGCAGCTGGCCTAACAGTA | |
| | TTCCTGAGTGAGGGTCGGATCCGGGCTGAGGTGCCGGGCAGTCCTCCTGTAGTGCTCC | |
| | CTGGGCGCTGGGATGATGGGCTCCGTCACCTGGTGATGCTCAGCTTCGGCCCTGACCA | |
| | GCTGCAGGACCTGGGGCAGCACGTGCACGTGGGTGGGAGGCTCCTTGCTGCCGACAGC | |
| | CAGCCCTGGGGTGGGCCCTTCCGAGGCTGCCTCCAGGACCTGCGACTCGATGGCTGCC | |
| | ACCTCCCCTTCTTTCCTCTGCCACTGGATAACTCAAGCCAGCCCAGCGAGCTCGGCGG | |
| | CAGGCAGTCCTGGAACCTCACTGCCGGCTGCGTCTCCGAGGACATGTGCAGTCCTGAC | |
| | CCCTGTTTCAATGGTGGGACTTGCCTCGTCACCTGGAATGACTTCCACTGTACCTGCC | |
| | CTGCCAATTTCACGGGGCCTACGTGTGCCCACCAGCTGTCGTGTCCCGGCCACCCCTG | |
| | TCTCCCACCTGCCACGTGTGAGGAGGTCCCTGATGCCTTTGTGTGTGTGGCGGAGGCC | |
| | ACGTTCCGCGAGGGTCCCCCCGCCGCGTTCAGCGGGCACAACGCGTCGTCAGGGCGCT | |
| | TGCTCGGCGGCCTGTCGCTGGCCTTTCGCACGCGCGACTCCGAGGCCTGGCTGCTGCG | |
| | TGCCGCGGCGGGCGCCCTGGAAGGCGTGTGGCTGGCGGTGCGCAATGGCTCGCTGGCG | |
| | GGGGGCGTGCGCGGAGGCCATGGCCTCCCCGGCGCTGTGCTGCCCATACCCGGCCCGC | |
| | GCGTGGCCGATGGTGCCTGGCACCGCGTGCGTCTCGCCATGGAGCGCCCGGCCGCCAC | |
| | CACCTCGCGCTGGCTGCTGTGGCTGGATGGTGCCGCCACCCCGGTGGCGCTGCGCGGC | |
| | CTGGCCAGTCACCTGGGCTTCCTGCAGGGCCCGGGTGCTGTGCGCATCCTGCTGGCTG | |
| | AGAACTTCACCGGCTGCTTGGGCCGCGTGGCGCTGGGCGGCCTGCCCCTGCCCTTGGC | |
| | GCGGCCCCGGCCCGGCGCGGCCCCTGGCGCCCGAGAGCACTTCGCGTCTTGGCCTGGG | |
| | ACGCCGGCCCCGATCCTCGGCTGCCGCGCCCCGCCCGTGTGTGCGCCCTCGCCCTGTC | |
| | TGCACGACGGTGCCTGCCGTGACCTCTTCGACGCCTTTGCCTGCGCCTGCGGCCCCGG | |
| | GTGGGAAGGCCCGCGCTGCGAAGCCCACGTCGACCCCTGTCACTCCGCCCCCTGCGCC | |
| | CGTGGCCGCTGTCACACGCACCCCGACGGCCGCTTCGAGTGCCGCTGCCCGCCTGGCT | |
| | TCGGGGGCCCGCGCTGCAGGTTGCCTGTCCCATCCAAGGAGTGCAGCCTGAATGTCAC | |
| | CTGCCTCGATGGCAGCCCATGTGAGCGTGGCTCTCCCGCTGCCAACTGCAGCTGCCTG | |
| | GAGGGTCTTGCTGGCCAGAGGTGTCAGGTCCCCACTCTCCCCTGTGAAGCCAACCCCT | |
| | GCTTGAATGGGGGCACCTGCCGGGCAGCTGGAGGGGTGTCTGAATGTATCTGCAATGC | |
| | CAGATTCTCCGGCCAGTTCTGTGAAGTGGCGAAGGGCCTGCCCCTGCCGCTGCCATTC | |
| | CCACTGCTGGAGGTGGCCGTACCTGCAGCCTGTGCCTGCCTCCTCCTCCTCCTCCTGG | |
| | GCCTCCTTTCAGGGATCCTGGCAGCCCGAAAGCGCCGCCAGTCTGAGGGCACCTACAG | |
| | CCCAAGCCAGCAGGAGGTGGCTGGGGCCCGGCTGGAGATCGACAGTGTCCTCAAGGTG | |
| | CCACCGGAGGAGAGACTCATCTAG | |
| | ORF Start: TTC at 19 | ORF Stop: GAG at 4183 |
| | SEQ ID NO:138 | 1388 aa MW at 145607.6 kD |
| NOV31a. | MKSALIPLVYRGGKRGPEMASPRARKPQSQCWDFTVHCATCLGKVTSLLCLYKTRTDW | |
| CG59289-01 Protein | LTPSAGIRIGRECSERFPTPGGNSLELCSEPKLSRVGQCQAQGRCSVPDSYCCNXGNS | |
| Sequence | LELCSEPKLSRVGQCQAQGTVPSEPPSACASDPCAPGTECQATESGGYTCGPMEPRGC | |
| | ATQPCHHGALCVPQGPDPTGFRCYCVPGFQGPRCELDIDECASRPCHHGATCRNLADR | |
| | YECHCPLGYAGVTCEMEVDECASAPCLHGGSCLDGVGSFRCVCAPGYGGTRCQLDLDE | |
| | CQSQPCAHGGTCHDLVNGFRCDCAGTGYEGTHCEREVLECASAPCEHNASCLEGLGSF | |
| | RCLCWPGYSGELCEVDEDECASSPCQHGGRCLQRSDPALYGGVQAAFPGAFSFRHAAG | |
| | FLCHCPPGFEGADCGVEVDECASRPCLNGGHCQDLPNGFQCHCPDGYAGPTCEEDVDE | |
| | CLSDPCLHGGTCSDTVAGYICRCPETwGGRDCSVQLTGCQGHTCPLAATCIPIFESGV | |
| | HSYVCHCPPGTHCPFCGQNTTFSVMAGSPIQASVPAGGPLGLALRFRTTLPAGTLATR | |
| | NDTKESLELALVAATLQATLWSYSTTVLVLRLPDLALNDGHWHQVEVVLHLATLELRL | |
| | WHEGCPARLCVASGPVALASTASATPLPAGISSAQLGDATFAGCLQDVRVDGHLLLPE | |
| | DLGENVLLGCERREQCRPLPCVHGGSCVDLWTHFRCDCARPHRGPTCADEIPAATFGL | |
| | GGAPSSASFLLQELPGPNLTVSFLLRTRESAGLLLQFANDSAAGLTVFLSEGRIRAEV | |
| | PGSPAVVLPGRWDDGLRHLVMLSFGPDQLQDLGQHVHVGGRLLAADSQPWGGPFRGCL | |
| | QDLRLDGCHLPFFPLPLDNSSQPSELGGRQSWNLTAGCVSEDMCSPDPCFNGGTCLVT | |
| | WNDFHCTCPANFTGPTCAQQLWCPGQPCLPPATCEEVPDGFVCVAEATFREGPPAAFS | |
| | GHNASSGRLLGGLSLAFRTRDSEAWLLRAAAGALEGVWLAVRNGSLAGGVRGGHGLPG | |
| | AVLPIPGPRVADGAWHRVRLANERPAATTSRWLLWLDGAATPVALRGLASDLGFLQGP | |
| | GAVRILLAENFTGCLGRVALGGLPLPLARPRPGAAPGAREHFASWPGTPAPILGCRGA | |
| | PVCAPSPCLHDGACRDLFDAFACACGPGWEGPRCEAHVDPCHSAPCARGRCHTHPDGR | |
| | FECRCPPGFGGPRCRLPVPSKECSLNVTCLDGSPCEGGSPAANCSCLEGLAGQRCQVP | |
| | TLPCEANPCLNGGTCRAAGGVSECICNARFSGQFCEVAKGLPLPLPFPLLEVAVPAAC | |
| | ACLLLLLLCLLSGILAARKRRQSEGTYSPSQQEVAGARLEMDSVLKVPPEERLI | |
| | SEQ ID NO:139 | 4200 bp |
| NOV31b, | TAACGAGGTCTAATTTAATTCTCCCAACAGCCTATGAAGTCAGCTCTGATACCCCTAG | |
| CG59289-02 DNA | TTTACAGAGGAGGTAAACGAGGCCCAGAGATGGCCAGTCCCAGAGCCAGGAAGCCCCA | |
| Sequence | GAGCCAGTGCTCGGATTTCACAGTACACTGTGCTACTTGCCTAGGCAAAGTCACGTCT | |
| | CTTCTCTGTCTCTACAAAACGAGGACAGACTGGCTTACTCCGAGCGCTGGTATAAGGA | |
| | TTGGAAGAGAATGCAGTGAGCGGTTCCCCACCCCAGGAGGTAACTCCCTGGAACTGTG | |
| | CTCTGAGCCCAAACTCTCAAGCGTTGGTCAGTGCCAGGCACAGGGTAGGTGCTCCGTC | |
| | CCTGACAGCTATTGTTGCAATCGAOCTAACTCCCTGGAACTGTGCTCTGAGCCCAAAC | |
| | TCTCAAGGGTTGGTCAGTGCCAGGCACAGCGGACGGTGCCTTCAGAGCCCCCCACTGC | |
| | CTGTGCCTCAGACCCGTGCGCTCCAGGGACCGAGTGCCAGGCTACCGAGAGTGGTGGC | |
| | TATACCTGTCCGCCCATGGAGCCCCGGGGCTGTGCCACCCAGCCATGCCACCACGGCG | |
| | CTCTGTGTGTGCCCCAGGGTCCAGATCCCACCGGCTTCCGCTGCTACTGCGTGCCGGG | |
| | TTTCCAGGGCCCACGCTGCGAGCTGGACATCGATGAGTGTGCATCCCGGCCGTGCCAC | |
| | CATGGGGCCACCTGCCGCAACCTGGCCGATCGCTACGAGTGCCATCGCCCCCTTGGCT | |
| | ATGCAGGCGTGACCTGCGAGACGGAGGTGGACGAGTGCGCCTCAGCGCCCTGCCTGCA | |
| | CGGGGCCTCGTGCCTGGACGGCGTGGGCTCCTTCCGCTGTGTGTGCGCGCCAGGCTAC | |
| | GGGGGCACCCGTTGCCAGCTCGACCTCGACCAGTGCCAGAGCCAGCCGTGCGCACATG | |
| | GGGGCACGTGCCACGACCTGGTCAACGGGTTCCCCTGCGACTGCGCGGGCACCGGCTA | |
| | CGAGGGCACGCACTGCGAGCGGGAGGTGCTGGAGTGCGCATCGGCGCCCTGCGAGCAC | |
| | AACGCGTCCTGCCTCGAGGGCCTCGGGAGCTTCCGCTGCCTCTGTTGGCCAGGCTACA | |
| | GCGGCGAGCTGTGCGAGGTGCACGAGGACGAGTGTGCATCGAGCCCCTGCCAGCATGG | |
| | GGGCCGATGCCTGCAGCGCTCTGACCCGGCCCTCTACGGGGGTGTCCAGGCCGCCTTC | |
| | CCTCGCGCCTTCAGCTTCCGCCATGCTGCGGGTTTCCTGTGCCACTGCCCTCCTGGCT | |
| | TTGAGGGAGCCGACTGCGGTGTGGAGGTGGACGAGTGTGCCTCACGGCCATGCCTCAA | |
| | CGGAGGCCACTGCCAGGACCTGCCCAATGGCTTCCAGTGTCACTGCCCAGATGGCTAC | |
| | GCAGGGCCGACATGTGACCAAGATGTGGATGAATGCCTGTCGGATCCCTGCCTGCACG | |
| | GCGGAACCTGCAGTGACACTGTGGCAGGCTATATCTGCAGGTGCCCAGAGACCTGGGG | |
| | TGGGCGCGACTGTTCTGTGCAGCTCACTGGCTGCCAGGGCCACACCTGCCCGCTGGCT | |
| | GCCACCTGCATCCCTATCTTCGAGTCTGGGGTCCACAGTTACGTCTGCCACTGCCCAC | |
| | CTGGTACCCATGGACCGTTCTGTGGCCAGAATACCACCTTCTCTGTGATGGCTGGGAG | |
| | CCCCATTCAGGCATCAGTGCCAGCTGGTGGCCCCCTGGGTCTGGCACTGAGGTTTCGC | |
| | ACCACACTGCCCGCTGGGACCTTGGCCACTCGCAATGACACCAAGGAAAGCTTGGAGC | |
| | TGGCATTGGTGGCAGCCACACTTCAGGCCACACTCTGGAGCTACAGCACCACTGTGCT | |
| | TGTCCTGAGACTGCCGGACCTGGCCCTAAACGATGGCCATTGGCACCAGGTGGAGGTT | |
| | GTGCTCCATCTAGCGACCCTGGAGCTACGGCTCTGGCATGAGGGTGCCCTGCCCGGC | |
| | TCTGTGTGGCCTCTGGTCCTGTGGCCCTGGCTTCCACGGCTTCGGCAACTCCGCTGCC | |
| | TGCCGGGATCTCCTCTGCCCAGCTGGGGGACGCGACCTTTGCAGGCTGCCTCCAGGAC | |
| | GTGCGTGTGGATGGCCACCTCCTGCTGCCTGAGGATCTCGGTGAGAACGTCCTCCTGG | |
| | GCTGTGAGCGCCGAGAGCAGTGCCGGCCTCTGCCTTGTGTCCACGGAGGGTCCTGTGT | |
| | GGATCTGTGGACTCATTTCCGTTGCGACTGTGCCCGGCCCCATAGAGGTCCCACGTGC | |
| | GCTGATGAGATTCCTGCTGCCACCTTTGGCTTGGGAGGCGCCCCAAGCTCTGCCTCCT | |
| | TTCTGCTCCAAGAGCTGCCAGGTCCCAACCTCACAGTGTCTTTCCTTCTCCGCACTCG | |
| | GGAGTCCGCTGGCCTGTTGCTCCAGTTTGCCAATGACTCCGCAGCTGGCCTIACAGTA | |
| | TTCCTGAGTCACCGTCGGATCCGGGCTGAGGTGCCGGGCAGTCCTGCTGTAGTGCTCC | |
| | CTGGGCGCTGGGATGATGGGCTCCGTCACCTGGTGATGCTCAGCTTCGGGCCTGACCA | |
| | GCTGCAGGACCTGGGGCAGCACGTGCACGTGGGTGGGAGGCTCCTTGCTGCCGACAGC | |
| | CAGCCCTGGGGTGGGCCCTTCCGAGGCTGCCTCCAGGACCTGCGACTCGATGGCTGCC | |
| | ACCTCCCCTTCTTTCCTCTGCCACTGGATAACTCAAGCCAGCCCAGCGAGCTCGGCGG | |
| | CACGCAGTCCTGGAACCTCACTGCGGGCTGCGTCTCCGAGCACATGTGCAGTCCTGAC | |
| | CCCTGTTTCAATGGTGGGACTTGCCTCGTCACCTGGAATGACTTCCACTGTACCTGCC | |
| | CTGCCAATTTCACGGGGCCTACGTGTGCCCAGCAGCTGTGGTGTCCCGCCCAGCCCTG | |
| | TCTCCCACCTGCCACGTGTGAGGAGGTCCCTGATGGCTTTGTGTGTGTGGCGGAGGCC | |
| | ACGTTCCGCGACCGTCCCCCCGCCGCGTTCAGCGGGCACAACGCGTCGTCAGGGCGCT | |
| | TGCTCGGCGGCCTGTCGCTGGCCTTTCGCACGCGCGACTCCGAGGCCTGGCTGCTGCG | |
| | TGCCGCGGCGGGCGCCCTGGAAGGCGTGTGGCTGGCGGTGCGCAATGGCTCGCTGGCG | |
| | GGGGGCGTGCGCGGAGGCCATGGCCTGCCCGGCGCTGTGCTGCCCATACCGGGGCCGC | |
| | GCGTGGCCGATGGTGCCTGGCACCGCGTGCGTCTGGCCATGGACCGCCCGGCGGCCAC | |
| | CACCTCGCGCTGGCTGCTGTGCCTCGATGGTGCCGCCACCCCGGTGGCGCTGCGCGGC | |
| | CTGGCCAGTGACCTGGGCTTCCTGCAGGGCCCGGCTGCTGTGCGCATCCTGCTGGCTG | |
| | AGAACTTCACCGGCTGCTTGGGCCGCGTGGCGCTGGGCGGCCTGCCCCTGCCCTTGGC | |
| | GCGGCCCCGGCCCGGCGCGGCCCCTGGCGCCCGAGAGCACTTCGCGTCTTGGCCTGGG | |
| | ACGCCGGCCCCGATCCTCGGCTCCCGCGGCGCGCCCGTGTGTGCGCCCTCGCCCTGTC | |
| | TGCACGACGGTGCCTGCCGTGACCTCTTCGACGCCTTTGCCTGCGCCTGCGGCCCGGG | |
| | GTGGGAAGGCCCGCGCTGCGAAGCCCACGTCGACCCCTGTCACTCCGCCCCCTGCGCC | |
| | CGTGGCCGCTGTCACACGCACCCCGACGGCCGCTTCGAGTGCCGCTGCCCGCCTGGCT | |
| | TCGCGGCCCCGCGCTGCAGGTTGCCTGTCCCATCCAAGGAGTGCAGCCTGAATGTCAC | |
| | CTCCCTCGATGGCAGCCCATGTGAGGGTGGCTCTCCCGCTGCCAACTGCAGCTGCCTG | |
| | GAGGGTCTTGCTGGCCAGAGGTGTCAGGTCCCCACTCTCCCCTGTGAAGCCAACCCCT | |
| | GCTTGAATGGGGGCACCTGCCGGGCAGCTGGAGGGGTGTCTGAATGTATCTGCAATGC | |
| | CAGATTCTCCGGCCAGTTCTGTGAAGTGGCGAAGGGCCTGCCCCTGCCGCTGCCATTC | |
| | CCACTGCTGGAGGTGGCCGTACCTGCAGCCTGTGCCTGCCTCCTCCTCCTCCTCCTGG | |
| | GCCTCCTTTCAGGGATCCTCGCAGCCCGAAAGCGCCGCCAGTCTGAGGGCACCTACAG | |
| | CCCAAGCCACCAGGACCTCGCTGGGGCCCGGCTGGAGATGGACAGTGTCCTCAAGGTG | |
| | CCACCGGAGGAGAGACTCATCTAG | |
| | ORF Start: ATG at 34 | ORF Stop: TAG at 4198 |
| | SEQ ID NO:140 | 1388 aa MW at 145800.8 kD |

| | |
|---|---|
| NOV31b, | MKSALIPLVYRGGKRGPEMASPRARKPQSQCWDFTVHCATCLGKVTSLLCLYKTRTDW |
| CG59289-02 Protein | LTPSAGIRIGRECSERFPTPGGNSLELCSEPKLSRVGQCQAQGRCSVPDSYCCNRGNS |
| Sequence | LELCSEPKLSRVGQCQAQGTVPSEPPSACASDPCAPGTECQATESGGYTCGPMEPRGC |
| | ATQPCHHGALCVPQGPDPTGFRCYCVPCFQGPRCELDIDECASRPCHHGATCRNLADR |
| | YECHRPLGYAGVTCETEVDECASAPCLHCASCLDGVGSFRCVCAPGYGGTRCQLDLDE |
| | CQSQPCAHGGTCHDLVNGFRCDCAGTGYEGTHCEREVLECASAPCEHNASCLEOLGSF |
| | RCLCWPGYSGELCEVDEDECASSPCQHGGRCLQRSDPALYGGVQAAFPGAFSFRHAAG |
| | FLCHCPPGFEGADCGVEVDECASRPCLNGGHCQDLPNGFQCHCPDGYAGPTCEEDVDE |
| | CLSDPCLHGGTCSDTVAGYICRCPETWGGRDCSVQLTGCQGHTCPLAATCIPIFESGV |
| | HSYVCHCPPGTHGPFCGQNTTFSVMAGSPIQASVPAGGPLGLALRFRTTLPAGTLATR |
| | NDTKESLELALVAATLQATLWSYSTTVLVLRLPDLALNDGHWHQVEVVLHLATLELRL |
| | WHEGCPARLCVASGPVALASTASATPLPAGISSAQLGDATFAGCLQDVRVDGHLLLPE |
| | DLGENVLLGCERREQCRPLPCVHGGSCVDLWTHFRCDCARPHRGPTCAEEIPAATFGL |
| | GGAPSSASFLLQELPGPNLTVSFLLRTRESAGLLLQFANDSAAGLTVFLSEGRIRAEV |
| | PGSPAVVLPGRWDDGLRHLVMLSFGPDQLQDLGQHVHVGGRLLAADSQPWGGPFRGCL |
| | QDLRLDGCHLPFFPLPLDNSSQPSELGGRQSWNLTAGCVSEDMCSPDPCFNGGTCLVT |
| | WNDFHCTCPANFTGPTCAQQLWCPGQPCLPPATCEEVPDGFVCVAEATFREGPPAAFS |
| | GHNASSGRLLGGLSLAFRTRDSEAWLLRAAAGALEGVWLAVRNGSLAGGVRGGHGLPG |
| | AVLPIPGPRVADGAWHRVRLAMERPAATTSRWLLWLDGAATPVALRGLASDLGFLQGP |
| | GAVRILLAENFTGCLGRVALGGLPLPLARPRPGAAPGAREHFASWPGTPAPILGCRGA |
| | PVCAPSPCLHDGACRDLFDAFACACGPGWEGPRCEAHVDPCHSAPCARGRCHTHPDGR |
| | FECRCPPGFCGPRCRLPVPSKECSLNVTCLDGSPCEGGSPAANCSCLEGLAGQRCQVP |
| | TLPCEANPCLNGGTCRAAGGVSECICNARFSGQFCEVAKGLPLPLPFPLLEVAVPAAC |
| | ACLLLLLLGLLSGILAARKRRQSEGTYSPSQQEVAGARLEMDSVLKVPPEERLI |

Sequence comparison of the above protein sequences yields the following sequence relationships shown in Table 31B.

TABLE 31B
Comparison of NOV31a against NOV31b and NOV31c.

| | NOV31a Residues/ | Identities/Similarities |
|---|---|---|
| Protein Sequence | Match Residues | for the Matched Region |
| NOV31b | 1 . . . 1388 | 1284/1388 (92%) |
| | 1 . . . 1388 | 1284/1388 (92%) |

Further analysis of the NOV31 a protein yielded the following properties shown in Table 31C.

TABLE 31C
Protein Sequence Properties NOV31a

| | |
|---|---|
| PSort | 0.7000 probability located in plasma membrane; 0.3000 |
| analysis: | probability located in microbody (peroxisome); 0.2000 |
| | probability located in endoplasmic reticulum (membrane); |
| | 0.1000 probability located in mitochondrial inner membrane |
| | No Known Signal Sequence Indicated |
| analysis: | |

A search of the NOV31a protein against the Geneseq database, a proprietary database that contains sequences published in patents and patent publication, yielded several homologous proteins shown in Table 31D.

TABLE 31D
Geneseq Results for NOV31a

| | | NOV31a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAB40530 | Human ORFX ORF294 polypeptide | 339 . . . 586 | 248/248 (100%) | e−160 |
| | sequence SEQ ID NO: 588 - Homo | 1 . . . 248 | 248/248 (100%) | |
| | sapiens, 248 aa. [WO200058473-A2, | | | |
| | 05-OCT-2000] | | | |
| AAU16970 | Human novel secreted protein, SEQ ID | 194 . . . 743 | 214/581 (36%) | e−118 |
| | 211 - Homo sapiens, 604 aa. | 19 . . . 557 | 308/581 (52%) | |
| | [WO200155441-A2, 02-AUG-2001] | | | |
| AAY06816 | Human Notch2 (humN2) protein | 145 . . . 541 | 166/434 (38%) | 2e−94 |
| | sequence - Homo sapiens, 2471 aa. | 799 . . . 1225 | 229/434 (52%) | |
| | [WO9904746-A2, 04-FEB-1999] | | | |
| AAB40789 | Human ORFX ORF553 polypeptide | 842 . . . 990 | 147/149 (98%) | 2e−92 |
| | sequence SEQ ID NO: 1106 - Homo | 1 . . . 149 | 148/149 (98%) | |
| | sapiens, 149 aa. [WO200058473-A2, | | | |
| | 05-OCT-2000] | | | |
| AAW49698 | Human Notch3 protein - Homo | 144 . . . 747 | 201/618 (32%) | 3e−92 |
| | sapiens, 2321 aa. [FR2751986-A1, | 354 . . . 888 | 260/618 (41%) | |
| | 06-FEB-1998] | | | |

In a BLAST search of public sequence databases, the NOV31a protein was found to have homology to the proteins shown in the BLASTP data in Table 31E.

TABLE 31E
Public BLASTP Results for NOV31a

| | | NOV31a | | |
|---|---|---|---|---|
| Protein | | Residues/ | Identities/ | |
| Accession | | Match | Similarities for the | Expect |
| Number | Protein/Organism/Length | Residues | Matched Portion | Value |
| AAL10682 | CRB1 ISOFORM II PRECURSOR - | 111 . . . 1388 | 448/1375 (32%) | 0.0 |
| | Homo sapiens (Human), 1406 aa. | 98 . . . 1406 | 664/1375 (47%) | |
| AAL65131 | PRECURSOR - Mus musculus | 37 . . . 1405 | 656/1434 (45%) | 0.0 |
| | (Mouse), 1405 aa. | | | |
| P82279 | Crumbs protein homolog 1 | 111 . . . 1313 | 421/1300 (32%) | 0.0 |
| | precursor - Homo sapiens (Human), | 98 . . . 1334 | 621/1300 (47%) | |
| | 1376 aa. | | | |
| Q9VC97 | CRB PROTEIN - Drosophila | 95 . . . 1312 | 430/1432 (30%) | e−174 |
| | melanogaster (Fruit fly), 2146 aa. | 552 . . . 1948 | 610/1432 (42%) | |
| P10040 | Crumbs protein precursor (95F) - | 95 . . . 1312 | 430/1444 (29%) | e−172 |
| | Drosophila melanogaster (Fruit fly), | 554 . . . 1951 | 613/1444 (41%) | |
| | 2139 aa. | | | |

PFam analysis indicates that the NOV31a protein contains the domains shown in the Table 31F.

TABLE 31F
Domain Analysis of NOV31a

| | | Identities/Similarities | |
|---|---|---|---|
| Pfam Domain | NOV31a Match Region | for the Matched Region | Expect Value |
| EGF: domain 1 of 16 | 86 . . . 120 | 8/50 (16%) | 52 |
| | | 20/50 (40%) | |
| EGF: domain 2 of 16 | 174 . . . 208 | 15/47 (32%) | 3.5e−05 |
| | | 27/47 (57%) | |
| EGF: domain 3 of 16 | 215 . . . 246 | 16/47 (34%) | 2.2e−09 |
| | | 27/47 (57%) | |
| EGF: domain 4 of 16 | 253 . . . 284 | 16/47 (34%) | 6.5e−08 |
| | | 27/47 (57%) | |
| EGF: domain 5 of 16 | 291 . . . 323 | 13/47 (28%) | 0.00014 |
| | | 27/47 (57%) | |
| EGF: domain 6 of 16 | 330 . . . 361 | 13/47 (28%) | 5.3e−07 |
| | | 25/47 (53%) | |
| EGF: domain 7 of 16 | 368 . . . 420 | 15/61 (25%) | 1.8 |
| | | 33/61 (54%) | |
| EGF: domain 8 of 16 | 427 . . . 458 | 17/47 (36%) | 5.1e−09 |
| | | 29/47 (62%) | |
| EGF: domain 9 of 16 | 465 . . . 496 | 15/47 (32%) | 2.9e−08 |
| | | 25/47 (53%) | |
| EGF: domain 10 of 16 | 503 . . . 538 | 12/47 (26%) | 0.19 |
| | | 25/47 (53%) | |
| laminin_G: domain 1 of 5 | 568 . . . 628 | 24/76 (32%) | 0.012 |
| | | 39/76 (51%) | |
| laminin_G: domain 2 of 5 | 679 . . . 694 | 7/16 (44%) | 0.2 |
| | | 15/16 (94%) | |
| EGF: domain 11 of 16 | 712 . . . 743 | 12/47 (26%) | 0.00015 |
| | | 25/47 (53%) | |
| laminin_G: domain 3 of 5 | 865 . . . 881 | 6/17 (35%) | 4.4 |
| | | 14/17 (82%) | |
| EGF: domain 12 of 16 | 914 . . . 945 | 16/47 (34%) | 6.1e−09 |
| | | 28/47 (60%) | |
| laminin_G: domain 4 of 5 | 1003 . . . 1068 | 21/79 (27%) | 0.00031 |
| | | 45/79 (57%) | |
| laminin_G: domain 5 of 5 | 1113 . . . 1128 | 7/16 (44%) | 0.86 |
| | | 12/16 (75%) | |
| EGF: domain 13 of 16 | 1163 . . . 1194 | 14/47 (30%) | 1.8e−07 |
| | | 24/47 (51%) | |
| EGF: domain 14 of 16 | 1201 . . . 1232 | 15/47 (32%) | 0.001 |
| | | 25/47 (53%) | |
| metalthio: domain 1 of 1 | 1198 . . . 1266 | 20/75 (27%) | 6.9 |
| | | 41/75 (55%) | |
| EGF: domain 15 of 16 | 1241 . . . 1273 | 11/47 (23%) | 4.5 |
| | | 17/47 (36%) | |
| EB: domain 1 of 1 | 1223 . . . 1273 | 15/60 (25%) | 4.8 |
| | | 33/60 (55%) | |
| EGF: domain 16 of 16 | 1280 . . . 1311 | 15/47 (32%) | 0.00045 |
| | | 23/47 (49%) | |

### Example 32

The NOV32 clone was analyzed, and the nucleotide and polypeptide sequences are shown in Table 32A.

TABLE 32A
NOV32 Sequence Analysis

| | SEQ ID NO:141 | 2855 bp |
|---|---|---|
| NOV32a | GAGGGAATGCGCGCACCTCACAGGCCCTGGGAGTGAGCTGGTGCCCGGCGACCTGGCA | |
| CG57111-01 DNA | CCCGCGCCTGGATATGGGGCGTCTACATCGTCCCAGGAGCAGCACCAGCTACACGAAC | |
| Sequence | CTGCCGCATCTGTTTCTGTTTTTCCTCTTCGTGGGACCCTTCAGCTGCCTCGGGAGTT | |
| | ACAGCCGGGCCACCGAGCTTCTGTACAGCCTAAACGAGGGACTACCCGCGGGGGTGCT | |
| | CATCGGCAGCCTGGCCGAGGACCTGCGGCTGCTGCCCAGGTCTGCAGGGAGGCCGGAC | |
| | CCGCAGTCGCAGCTGCCAGAGCGCACCGGTGCTCAGTGGAACCCCCCTCTCTCCTTCA | |
| | GCCTGGCCTCCCGGGGACTGAGTGGCCAGTACGTGACCCTAGACAACCGCTCTGGGGA | |
| | GCTGCACACTTCACCTCAGGAGATCGACAGGCAGGCCCTGTGTGTTCAAGGGGGTGGA | |
| | GGGACTGCGTGGAGCGGCAGCGTTTCCATCTCCTCCTCTCCTTCTGACTCTTGTCTTT | |
| | TGCTGCTGGATGTGCTTGTCCTGCCTCAGGAATACTTCAGGTTTGTGAAGGTGAAGAT | |
| | CGCCATCAGAGACATCAATGACAACGCCCCGCAGTTCCCTGTTTCCCAGATCTCGGTG | |
| | TCGGTCCCGGAAAATGCACCTGTAAACACCCGACTGGCCATAGAGCATCCTGCTGTGG | |
| | ACCCAGATGTTGGCATTAATGGCGTTCAGACCTATCGCTTACTGGACTACCATGGTAT | |
| | GTTCACCCTGGACGTGGAGGAGAATGAGAATGGGGAGCGCACCCCCTACCTAATTCTC | |
| | ATGCGTGCTTTGGACAGGGAAACCCAGGACCAGTATGTGAGCATCATCACAGCTGAGG | |
| | ATGGTGGCTCTCCACCACTTTTGGGCAGTGCCACTCTCACCATTGGCATCAGTGACAT | |
| | TAATGACAATTGCCCTCTCTTCACAGACTCACAAATCAATGTCACTGTGTATGGGAAT | |
| | GCTACAGTGGGCACCCCAATTGCAGCTGTCCAGGCTGTGGATAAAGACTTGGGGACCA | |
| | ATGCTCAAATTACTTATTCTTACAGTCAGAAAGTTCCACAAGCATCTAAGGATTTATT | |
| | TCACCTGGATGAAAACACTGGAGTCATTAAACTTTTCAGTAAGATTGGAGGAAGTGTT | |
| | CTGGAGTCCCACAAGCTCACCATCCTTGCTAATGGACCAGGCTGCATCCCTGCTGTPA | |
| | TCACTGCTCTTGTGTCCATTATTAAAGTTATTTTCAGACCCCCTGAAATTGTCCCTCG | |
| | TTACATAGCAAACGAGATAGATGGTGTTGTTTATCTGAAAGAACTGGAACCCGTTAAC | |
| | ACTCCCATTGCGTTTTTCACCATAAGAGATCCAGAAGGTAAATACAAGGTTAACTGCT | |
| | ACCTGGATGGTGAAGGGCCGTTTAGGTTATCACCTTACAAACCATACAATAATGAATA | |
| | TTTACTAGAGACCACAAAACCTATGGACTATGAGCTACAGCAGTTCTATGAAGTAGCT | |
| | GTGGTGGCTTGGAACTCTGAGGGATTTCATGTCAAAAGGGTCATTAAAGTGCAACTTT | |
| | TAGATGACAATGATAATGCTCCAATTTTCCTTCAACCCTTAATAGAACTAACCATCGA | |
| | AGAGAACAACTCACCCAATGCCTTTTTGACTAAGCTGTATGCTACAGATGCCGACAGC | |
| | GAGGAGAGAGGCCAAGTTTCATATTTTCTGGGACCTGATGCTCCATCATATTTTTCCT | |
| | TAGACAGTGTCACAGGAATTCTGACAGTTTCTACTCACCTGGACCGAGAAGAGAAAGA | |
| | AAAGTACAGATACACTGTCAGAGCTGTTGACTGTGGGAAGCCACCCAGAGAATCAGTA | |
| | GCCACTGTGGCCCTCACAGTGTTGGATAAAAATGACAACAGTCCTCGGTTTATCAACA | |
| | AGGACTTCAGCTTTTTTGTGCCTGAAAACTTTCCAGGCTATGGTGAGATTGGAGTAAT | |
| | TAGTGTAACAGATGCTGACGCTGGACGAAATGGATGGGTCGCCCTCTCTGTGGTGAAC | |
| | CAGAGTGATATTTTTGTCATAGATACAGGAAAGGGTATGCTGAGGGCTAAAGTCTCTT | |
| | TGGACAGAGAGCAGCAAAGCTCCTATACTTTGTGCGTTGAAGCTGTTGATGGGGGTGA | |
| | GCCTGCCCTCTCCTCTACAGCAAAAATCACAATTCTCCTTCTAGATATCAATGACAAC | |
| | CCTCCTCTTGTTTTGTTTCCTCAGTCTAATATGTCTTATCTGTTAGTACTGCCTTCTA | |
| | CTCTGCCAGGCTCCCCGGTTACAGAAGTCTATGCTGTCGACAAAGACACACGCATGAA | |
| | TCCTGTCATAGCTTACAGCATCATAGGGAGAAGAGGTCCTAGGCCTGAGTCCTTCAGG | |
| | ATTGACCCTAAAACTGGCAACATTACTTTGGAAGAGGCATTGCTGCAGACAGATTATG | |
| | GGCTCCATCGCTTACTGGTGAAAGTGAGTGATCATGGTTATCCCCAGCCTCTCCACTC | |
| | CACACTCATGGTGAACCTATTTGTCAATGACACTGTCAGTAATGAGAGTTACATTGAG | |
| | AGTCTTTTAAGAAAAGAACCAGAGATTAATATAGAGGAGAAAGAACCACAAATCTCAA | |
| | TAGAACCGACTCATAGGAAGGTACAATCTCTGTCTTGTATGCCCACCTTAGTAGCTCT | |
| | GTCTGTAATAAGCTTGGGTTCCATCACACTGGTCACAGGGATCGGCATATACATCTGT | |
| | TTAAGGAAAGGGGAAAAGCATCCCAGGGAAGATGAAAATTTGGAAGTACAGATTCCAC | |
| | TGAAAGGAAAAATTGACTTGCATATGCGAGAGAGAAAGCCAATGGATATTTCTAATAT | |
| | TTGATATTTCATG | |
| | ORF Start: GGG at 3 | ORF Stop: TGA at 2844 |
| | SEQ ID NO:142 | 947 aa MW at 104405.1 kD |

| | | |
|---|---|---|
| NOV32a, | GNARSSQALGVSWCPATWHPRLDMGRLHRPRSSTSYRNLPHLFLFFLPVGPFSCLGSY | |
| CG57111-01 | Protein | SRATELLYSLNEGLPAGVLIGSLAEDLRLLPRSAGRPDPQSQLPERTGAEWNPPLSFS |
| Sequence | LASRGLSGQYVTLDNRSGELHTSAQEIDREALCVEGGCGTAWSGSVSISSSPSDSCLL | |
| | LLDVLVLPQEYFRFVKVKIAIRDINDNAPQFPVSQISVWVPENAPVNTRLAIEHPAVD | |
| | PDVGILNGVQTYRLLDYHGMFTLDVEENENGERTPYLIVMGALDRETQDQYVSIITAED | |
| | GGSPPLLGSATLTIGISDINIJNCPLFTDSQINVTVYGNATVGTPIAAVQAVDKDLGTN | |
| | AQITYSYSQKVPQASKDLFHLDENTGVIKLFSKIGGSVLESHIKLTILANGPCCIPAVI | |
| | TALVSIIKVIFRPPEIVPRYIANEIDGVVYLKELEPVNTPIAFFTIRDPEGKYKVNCY | |
| | LDGEGPFRLSPYKPYNNEYLLETTKPMDYELQQFYEVAVVAWNSEGFHVKRVIKVQLL | |
| | DDNDNAPIFLQPLIELTIEENNSPNAFLTKLYATDADSEERGQVSYFLGPDAPSYFSL | |
| | DSVTGILTVSTQLDREEKEKYRYTVRAVDCGKPPRESVATVALTVLDKNDNSPRFINK | |
| | DFSFFVPENFPGYGEIGVISVTDADAGRNGWVALSVVNQSDIFVIDTGKGMLRAKVSL | |
| | DREQQSSYTLWVEAVDGGEPALSSTAKITILLLDINDNPPLVLFPQSNMSYLLVLPST | |
| | LPGSPVTEVYAVDKDTGMNAVIAYSIIGRRGPRPESFRIDPKTGNITLEEALLQTDYG | |
| | LHRLLVKVSDHGYPEPLHSTVMVNLFVNDTVSNESYIESLLRKEPEINIEEKEPQISI | |
| | EPTHRKVESVSCMPTLVALSVISLGSITLVTGMGIYICLRKGEKHPREDENLEVQIPL | |
| | KGKIDLHMRERKPMDISNI | |

Further analysis of the NOV32a protein yielded the following properties shown in Table 32B.

TABLE 32B
Protein Sequence Properties NOV32a

| | |
|---|---|
| PSort | 0.6400 probability located in plasma membrane; 0.4000 |
| analysis: | probability located in Golgi body; 0.3000 |
| | probability located in endoplasmic reticulum (membrane); |
| | 0.0300 probability located in mitochondrial inner membrane |
| SignalP | Cleavage site between residues 57 and 58 |
| analysis: | |

A search of the NOV32a protein against the Geneseq database, a proprietary database that contains sequences published in patents and patent publication, yielded several homologous proteins shown in Table 32C.

TABLE 32C
Geneseq Results for NOV32a

| | | NOV32a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAU19645 | Human novel extracellular matrix | 551 . . . 947 | 396/397 (99%) | 0.0 |
| | protein, Seq ID No 295 - Homo | 1 . . . 397 | 397/397 (99%) | |
| | sapiens, 397 aa. [WO200155368-A1, | | | |
| | 02-AUG-2001] | | | |
| AAM79103 | Human protein SEQ ID NO 1765 - | 46 . . . 858 | 347/820 (42%) | 0.0 |
| | Homo sapiens, 1021 aa. | 8 . . . 791 | 506/820 (61%) | |
| | [WO200157190-A2, 09-AUG-2001] | | | |
| AAM80087 | Human protein SEQ ID NO 3733 - | 127 . . . 868 | 325/747 (43%) | e−175 |
| | Homo sapiens, 1090 aa. | 5 . . . 728 | 475/747 (63%) | |
| | [WO200157190-A2, 09-AUG-2001] | | | |
| AAU19807 | Human novel extracellular matrix | 553 . . . 839 | 287/287 (100%) | e−164 |
| | protein, Seq ID No 457 - Homo | 1 . . . 287 | 287/287 (100%) | |
| | sapiens, 288 aa. [WO200155368-A1, | | | |
| | 02-AUG-2001] | | | |
| AAM31106 | measuring placental gene expression - | 4 . . . 611 | 398/612 (64%) | e−148 |
| | Homo sapiens, 831 aa. | | | |
| | [WO200157272-A2, 09-AUG-2001] | | | |

In a BLAST search of public sequence databases, the NOV32a protein was found to have homology to the proteins shown in the BLASTP data in Table 32D.

TABLE 32D
Public BLASTP Results for NOV32a

| | | NOV32a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NRT9 | PROTOCADHERIN 13 - Homo | 1 . . . 947 | 947/947 (100%) | 0.0 |
| | sapiens (Human), 947 aa (fragment). | 1 . . . 947 | 947/947 (100%) | |
| Q9HC56 | ROTOCADHERIN-9 - Homo sapiens | 45 . . . 862 | 364/823 (44%) | 0.0 |
| | (Human), 1203 aa. | 6 . . . 797 | 507/823 (61%) | |
| Q95KD8 | HYPOTHETICAL 113.7 KDA | 45 . . . 862 | 363/823 (44%) | 0.0 |
| | PROTEIN - Macaca fascicularis (Crab | 6 . . . 797 | 507/823 (61%) | |
| | eating macaque) (Cynomolgus | | | |
| | monkey), 1032 aa. | | | |
| Q9H4E0 | PROTOCADHERIN - Homo sapiens | 46 . . . 858 | 347/820 (42%) | 0.0 |
| | (Human), 1025 aa. | 8 . . . 791 | 506/820 (61%) | |
| Q9BZA6 | PROTOCADHERIN 11 - Homo | 46 . . . 858 | 347/820 (42%) | 0.0 |
| | sapiens (Human), 1337 aa. | 8 . . . 791 | 506/820 (61%) | |

PFam analysis indicates that the NOV32a protein contains the domains shown in Table 32E.

TABLE 32E
Domain Analysis of NOV32a

| | | ldentities/ | |
|---|---|---|---|
| | | Similarities | |
| Pfam Domain | NOV32a Match Region | for the Matched Region | Expect Value |
| cadherin: domain 1 of 5 | 210 . . . 307 | 38/111 (34%) | 1.5e−08 |
| | | 72/111 (65%) | |
| cadherin: domain 2 of 5 | 321 . . . 413 | 27/110 (25%) | 0.00028 |
| | | 67/110 (61%) | |
| cadherin: domain 3 of 5 | 536 . . . 626 | 32/107 (30%) | 1.1e−18 |
| | | 68/107 (64%) | |
| cadherin: domain 4 of 5 | 640 . . . 729 | 70/107 (65%) | 9.1e−20 |
| cadherin: domain 5 of 5 | 747 . . . 840 | 37/112 (33%) | 6.6e−11 |
| | | 69/112 (62%) | |

### Example 33

The NOV33 clone was analyzed, and the nucleotide and polypeptide sequences are shown in Table 33A.

TABLE 33A
NOV33 Sequence Analysis

| | SEQ ID NO:143 | 1289 bp |
|---|---|---|
| NOV33a, | TCCCAGCCTCGCAGCCGGCCTGAGCCGCCATGCGCGGGAAGTTGCTGCCGCTGGCCGG | |
| CG59363-01 DNA | CCTCTACCTGGTGCAGGGCCTGCCCTACGGGCTCCAGTCCGGCCTCCTGCCAGTCCTG | |
| Sequence | CTGCGTGCCGGCGGCCTCTCGCTGACGCGCGTGGGGCTGGCCAAGGTTCTGTACGCTC | |
| | CGTGCCTGCTCAAGCTGGCTTGGGCCCCGCTGGTGGACGCGCAGGGCTCGGCGAGGGC | |
| | CTGCGTCACCCGCAGCACCGCGGGCCTCGGCCTGGTCTGTGGCCTGCTTGCCGGGCTG | |
| | CCCCCTCCTGGAGCTGGCCAGGCCCGGCTGCCCGCCGCTGTGGCGGGGTTGCTGCTGT | |
| | TGTTGAACCTGGGTGCCGCCATGCAGGATGTGGCCCTGGACGCGCTGGCTGTGCAGCT | |
| | GCTGGAGCCGCCCGAACTGCGGCCGGGCAATACCGTGCAGGTGGTCGCGTACAAGCTG | |
| | GGGGCCGCGCTAGCTGGGGGCGCGCTCCTGGCGCTGCTGCCCACCTTCTCGTGGCCGC | |
| | AACTCTTTCTGCTCCTGGCTGCCACCTACTGGCTGGCCGCGGCCCTGGCCTGGGCTGC | |
| | ACCAGCCCTGCCGCGGCTCCCACAGCAGCCCCCTTCCGAGCAGCGTCCCCACACCGCG | |
| | CACCTTCTGCGGGACGTGCTAGCCGTGCCGGGCACCGTGTGGACGGCAGGCTTTGTGC | |
| | TCACCTACAAGCTGGGTGAGCAGGGTGCCAGCAGCCTGTTTCCTCTTCTCCTGCTGGA | |
| | CCACGGCGTTTCTGCTCCCGAGTTGGGACTGTGGAATGGTGTGGGTGCTGTGGTCTGC | |
| | TCCATCGCTGGCTCCTCCCTGGGTGGGACCTTGCTGGCCAAGCACTGGAAACTGCTGC | |
| | CTCTGTTGAGGTCGGTGCTGCGCTTCCGCCTCGGGGGCCTAGCCTGTCAGACTGCCTT | |
| | GGTCTTCCACCTGGACACCCTGGGGGCCAGCATGGACGCTGGCACAATCTTGAGAGGT | |
| | TCAGCCTTGCTGAGCCTATGTCTGCACCACTTCTTGGCAGGCCTGGTCACCACAGTCA | |
| | CCTTCACTGGGATGATGCGCTGCAGCCAGCTGGCCCCCAGGGCCCTGCAGGCCACACA | |
| | CTACAGCCTTCTCGCCACGCTGGAGCTGCTGGGGAAGCTGCTGCTGGGCACTCTGGCC | |
| | GGAGGCCTGGCTGATGGGTTGGGGCCACATCCCTGCTTCTTGCTCCTGCTCATCCTCT | |
| | CTGCCTTTCCCGTTCTCTACCTGGACCTAGCACCCAGCACCTTTCTCTGAGCTGAGTG | |
| | GCTGGAGTGGTCA | |
| | ORF Start: ATG at 30 | ORF Stop: TGA at 1266 |
| | SEQ ID NO:144 | 412 aa MW at 42695.2 kD |
| NOV33a, | MRGKLLPLAGLYLVQGLPYCLQSGLLPVLLRAGGLSLTRVGLAKVLYAPWLLKLAWAP | |
| CG59363-01 Protein | LVDAQGSARAWVTRSTAGLGLVCGLLAGLPPPGACQAGLPAAVAGLLLLLNLGAAMQD | |
| Sequence | VALDALAVQLLEPAELGPGNTVQVVAYKLGAALAGGALLALLPTFSWPQLFLLLAATY | |
| WLAAALAWAAPALRRLPQQPPSEQRPHTAHLLRDVLAVPGTVWTAGFVLTYKLGEQCA | | |
| | SSLFPLLLLDHGVSAPELGLWNGVGAVVCSIAGSSLGGTLLAKHWKLLPLLRSVLRFR | |
| | LGGLACQTALVFHLDTLGASMDAGTILRGSALLSLCLQHFLGGLVTTVTFTGMMRCSQ | |
| | LAPRALQATHYSLLATLELLGKLLLGTLAGGLADGLGPHPCFLLLLILSAFPVLYLDL | |
| | APSTFL | |
| | SEQ ID NO:145 | 1256 bp |
| NOV33b, | CATGCGCGGGAAGTTGCTGCCGCTGGCCGGCCTCTACCTGGTGCAGGGCCCGCCCTAC | |
| CG59363-02 DNA | GGGCTCCAGTCCGGCCTCCTGCCAGTGCTGCTGCGTGCCGGCGGCCTCTCGCTGACGC | |
| Sequence | CCGTGGGGCTGGCCAAGGTTCTGTACGCTCCGTGGCTGCTCAAGCTGGCTTGGGCCCC | |
| GCTGGTGGACGCGCAGGGCTCGGCGAGGGCCTGGGTGACGCGCAGCACGGCGGGCCTC | | |
| | GGCCTGGTGTGTGGGCTGCTTGCCGGGCTGCCCCCTCCTGGAGCTGGCCAGGCCGCGC | |
| | TGCCCGCCGCTGTGGCGGGGTTGCTGCTGTTGTTGAACCTGGGTGCCGCCATGCAGGA | |
| | TGTGGCCCTGGACGCGCTGGCTGTGCAGCTGCTGGAGCCGGCCGAACTGGGGCCGGGC | |
| | AATACCGTGCAGGTGGTCGCGTACAAGCTCGGGGCCGCGCTAGCTGGGGGCGCGCTGC | |
| | TGGCGCTGCTGCCCACCTTCTCGTGGCCGCAACTCTTTCTGCTCCTGGCTGCCACCTA | |
| | CTGGCTGGCCGCGGCCCTGGCCTGGGCTGCACCAGCCCTGCGGCGGCTCCCACAGCAG | |
| | CCCCCTTCCGAGCAGCGTCCCCACACCGCGCACCTTCTGCGGGACGTGCTAGCCGTGC | |
| | CGGGGACCGTGTGGACGGCAGGCTTTGTGCTCACCTACAAGCTGGGTGAGCAGGGTGC | |
| | CAGCAGCCTGTTTCCTCTTCTCCTGCTGCACCACGGCGTTTCTGCTCCCCAGTTGGGA | |
| | CTGTGGAATGGTGTGGGTGCTGTGGTCTCCTCCATCGCTGGCTCCTCCCTGGGTCGCA | |
| | CCTTGCTGGCCAAGCACTGGAAACTGCTGCCTCTGTTGAGGTCGGTGCTGCGCTTCCG | |
| | CCTCGGGCGCCTAGCCTGTCAGACTGCCTTGGTCTTCCACCTGGACACCCTGGGGGCC | |
| | AGCATGGACGCTGGCACAATCTTGAGAGGGTCAGCCTTGCTGAGCCTATGTCTGCAGC | |
| | ACTTCTTGGGAGCCCTGGTCACCACAGTCACCTTCACTGGGATGATGCGCTGCAGCCA | |
| | GCTGGCCCCCAGGGCCCTGCAGGCCACACACTACAGCCTTCTGGCCACGCTGGAGTTG | |
| | CTGGGGAAGCTGCTGCTGGGCACTCTGGCCGGAGGCCTGGCTGATGGGTTGGGGCCAC | |
| | ATCCCTGCTTCTTGCTCCTGCTCATCCTCTCTGCCTTTCCCGTTCTGTACCTGGACCT | |
| | AGCACCCAGCACCTTTCTCTGAGCTGAGTGGCTGGAGT | |
| | ORF Start: ATG at 2 | ORF Stop: TGA at 1238 |
| | SEQ ID NO:146 | 412 aa MW at 42679.2 kD |
| NOV33b, | MRGKLLPLAGLYLVQGPPYGLQSGLLPVLLRAGGLSLTRVGLAKVLYAPWLLKLAWAP | |
| CG59363-02 Protein | LVDAQGSARAWVTRSTAGLGLVCGLLAGLPPPGAGQAGLPAAVAGLLLLLNLGAAMQD | |
| Sequence | VALDALAVQLLEPAELGPGNTVQVVAYKLGAALAGGALLALLPTFSWPQLFLLLAATY | |
| | WLAAALAWAAPALRRLPQQPPSEQRPHTAHLLRDVLAVPGTVWTAGFVLTYKLGEQGA | |
| | SSLFPLLLLDHGVSAPELGLWNGVGAVVCSIAGSSLGGTLLAKHWKLLPLLRSVLRFR | |
| | LGGLACQTALVFHLDTLGASMDAGTILRGSALLSLCLQHFLGGLVTTVTFTGMMRCSQ | |
| | LAPRALQATHYSLLATLELLGKLLLGTLACGLADCLGPHPCFLLLLILSAFPVLYLDL | |
| | APSTFL | |
| | SEQ ID NO:147 | 1186 bp |
| NOV33C, | TCCCAGCCTCGCAGCCGGCCTGAGCCGCCATGCGCGGGAAGTTGCTGCCGCTGGCCGG | |
| CG59363-03 DNA | CCTCTACCTGGTGCAGGGCCTGCCCTACGGGCTCCAGTCCGGCCTCCTGCCAGTGCTG | |
| Sequence | CTCCGTGCCGGCGGCCTCTCGCTGACGCCCGTGGGGCTGGCCAAGGTTCTGTACGCTC | |
| | CGTGGCTGCTCAAGCTGGCTTGCGCCCCGCTGGTGGACGCGCAGGGCTCGGCGAGGGC | |
| | CTGGGTGACGCGCAGCACGGCGGGCCTGGGCCTGGTGTGTGGGCTGCTTGCCGGGCTG | |
| | CCCCCTCCTGGAGCTGCCCAGGCCGGGCTGCCCGCCGCTGTGGCGGGGTTGCTGCTGT | |
| | TGTTGAACCTGGGTGCCGCCATGCAGGATGTGGCCCTGCACGCCCTGGCTGTGCAGCT | |
| | GCTGGAGCCGGCCGAACTGGGGCCGGGCAATACCGTGCAGGTGGTCGCGTACAAGCTG | |
| | GGGGCCGCGCTAGCTGGGGGCGCGCTGCTGGCGCTGCTGCCCACCTTCTCGTGGCCGC | |
| | AACTCTTTCTGCTCCTGGCTGCCACCTACTGGCTGGCCGCGGCCCTGGCCTGGGCTGC | |
| | ACCAGCCCTGCGGCGGCTCCCACAGCAGCCCCCTTCCGAGCAGCGTCCCCACACCGCG | |
| | CACCTTCTGCGGGACGTGCTAGCCGTGCCGGGGACCGTGTGGACGGCAGGCTTTGTGC | |
| | TCACCTACAAGCTGGGTGAGCAGGGTGCCAGCAGCCTGTTTCCTCTTCTCCTGCTGGA | |
| | CCACGGCGTTTCTGCTCCCGAGTTGGGACTGTGGAATGGTGTGGGTGCTGTGGTCTGC | |
| | TCCATCGCTGCCTCCTCCCTGGGTCGGACCTTGCTGGCCAAGCACTGGAAACTGCTGC | |
| | CTCTGTTGAGGTCGGTGCTGCGCTTCCGCCTCGGGGGCCTAGCCTGTCAGACTGCCTT | |
| | GGTCTTCCCCAGGGCCCTGCAGGCCACACACTACAGCCTTCTGGCCACGCTCGAGCTG | |
| | CTGGGGAAGCTGCTGCTGGGCACTCTGCCCGGAGGCCTGGCTGATGGCTTGCGGCCAC | |
| | ATCCCTGCTTCTTGCTCCTGCTCATCCTCTCTGCCTTTCCCGTTCTGTACCTGGACCT | |
| | AGCACCCAGCACCTTTCTCTGAGCTGAGTGGCTGGAGTGGTCAATAAAGCCACATGTC | |
| | CTGTGGCCCAGATGTCTCTGTGCCTG | |
| | ORF Start: ATG at 30 | ORF Stop: TGA at 1122 |
| | SEQ ID NO:148 | 364 aa MW at 37703.4 kD |

| | |
|---|---|
| NOV33C, | MRGKLLPLAGLYLVQGLPYGLQSGLLPVLLRAGGLSLTRVGLAKVLYAPWLLKLAWAP |
| CG59363-03 Protein | LVDAQCSARAWVTRSTAGLGLVCGLLAGLPPPGAGQAGLPAAVAGLLLLLNLGAAMQD |
| Sequence | VALDALAVQLLEPAELGPGNTVQVVAYKLGAALAGGALLALLPTFSWPQLFLLLAATY |
| | WLAAALAWAAPALRRLPQQPPSEQRPHTAHLLRDVLAVPGTVWTAGFVLTYKLGEQGA |
| | SSLFPLLLLDHGVSAPELGLWNGVGAVVCSIAGSSLGGTLLAKHWKLLPLLRSVLRFR |
| | LGGLACQTALVFPRALQATHYSLLATLELLGKLLLGTLAGGLADGLGPHPCFLLLLIL |
| | SAFPVLYLDLAPSTFL |

Sequence comparison of the above protein sequences yields the following sequence relationships shown in Table 33B.

TABLE 33B
Comparison of NOV33a against NOV33b through NOV33c.

| | NOV33a Residues/ | Identities/Similarities |
|---|---|---|
| Protein Sequence | Match Residues | for the Matched Region |
| NOV33b | 1 . . . 412 | 253/412 (61%) |
| | 1 . . . 412 | 253/412 (61%) |
| NOV33c | 1 . . . 412 | 221/412 (53%) |
| | 1 . . . 364 | 221/412 (53%) |

Further analysis of the NOV33a protein yielded the following properties shown in Table 33C.

TABLE 33C
Protein Sequence Properties NOV33a

| | |
|---|---|
| PSort | 0.6400 probability located in plasma membrane; 0.4600 |
| analysis: | probability located in Golgi body; 0.3700 probability located |
| | in endoplasmic reticulum (membrane); 0.1000 |
| | probability located in endoplasmic reticulum (lumen) |
| SignalP | Cleavage site between residues 63 and 64 |
| analysis: | |

A search of the NOV33a protein against the Geneseq database, a proprietary database that contains sequences published in patents and patent publication, yielded several homologous proteins shown in Table 33D.

TABLE 33D
Geneseq Results for NOV33a

| | | NOV33a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAW70898 | Acetyl-coenzyme A transporter (AT) | 5 . . . 252 | 72/280 (25%) | 2e−10 |
| | protein - Homo sapiens, 549 aa. | 75 . . . 347 | 116/280 (40%) | |
| | [US5851788-A, 22-DEC-1998] | | | |
| AAW69948 | (AT-1) protein - Homo sapiens, 549 aa. | 75 . . . 347 | 116/280 (40%) | 2e−10 |
| | [WO9833816-A1, 06-AUG-1998] | | | |

In a BLAST search of public sequences databases, the NOV33a protein was found to have homology to the proteins shown in the BLASTP data in Table 33E.

TABLE 33E
Public BLASTP Results for NOV33a

| | | NOV33a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96ES6 | SIMILAR TO RIKEN CDNA | 1 . . . 412 | 412/412 (100%) | 0.0 |
| | 2310010G13 GENE - Homo sapiens | 1 . . . 412 | 412/412 (100%) | |
| | (Human), 412 aa. | | | |
| AAH19171 | SIMILAR TO RIKEN CDNA | 1 . . . 409 | 321/409 (78%) | 0.0 |
| | 2310010G13 GENE - Mus musculus | 1 . . . 409 | 351/409 (85%) | |
| | (Mouse), 412 aa. | | | |
| Q9D7F8 | 2310010G13RIK PROTEIN - Mus | 1 . . . 409 | 317/409 (77%) | e−180 |
| | musculus (Mouse), 412 aa. | 1 . . . 409 | 351/409 (85%) | |
| CAD13682 | TRANSPORT TRANSMEMBRANE | 11 . . . 404 | 120/403 (29%) | 8e−33 |
| | PROTEIN - Ralstonia solanacearum | 21 . . . 399 | 187/403 (45%) | |
| | (Pseudomonas solanacearum), 426 aa. | | | |
| Q92SY4 | PUTATIVE TRANSPORT PROTEIN - | 8 . . . 399 | 115/400 (28%) | 2e−25 |
| | Rhizobium meliloti (Sinorhizobium | 20 . . . 386 | 172/400 (42%) | |
| | meliloti), 421 aa. | | | |

PFam analysis indicates that the NOV33a protein contains the domains shown in the Table 33F.

TABLE 33F
Domain Analysis of NOV33a

| | | Identities/ | |
|---|---|---|---|
| | | Similarities | |
| | NOV33a Match | for the Matched | Expect |
| Pfam Domain | Region | Region | Value |
| Bac_Ubq_Cox: domain | 24 . . . 55 | 16/32 (50%) | 3.4 |
| 1 of 1 | | 26/32 (81%) | |
| BT1: domain 1 of 1 | 15 . . . 411 | 99/602 (16%) | 8.7 |
| | | 251/602 (42%) | |
| sugar_tr: domain 1 of 1 | 3 . . . 411 | 81/522 (16%) | 8.5 |
| | | 243/522 (47%) | |

### Example 34

The NOV34 clone was analyzed, and the nucleotide and polypeptide sequences are shown in Table 34A.

TABLE 34A
NOV34 Sequence Analysis

| | SEQ ID NO:149 | 1020 bp |
|---|---|---|
| NOV34a, | ATGGCGGGGCAGTGGGAGGCGGGGCCCAGTCGGAGAGCGGGGCGGATTTGGGCGGGTG | |
| CG59301-01 DNA | AGGGGAAATCGGCCTGCCCCTACGGAAATCCCGTTTCTTTATACACCCCGTCCTGGCT | |
| Sequence | GGGCGGTGTCTTCCCCGTGGAAGAGAGGGTCCGGGAGGTCCGTGGGGCGGAACAGTCC | |
| | GAGGGCTTCGGGAGTCAGCCCGGGTGGATTTGTTTCGTGGCTCTCGTTGCTGGCTGGG | |
| | ACGGCCGTGTCCCCGTGTTGTTCCGGAGATCGAGGCCAACGGATGCCGTACTCTGTCC | |
| | TCGCCGGGTGCTCAGTGAGGACCCACAGCTGGGCAGGGACCCTGGGAGGCCCCTGAGC | |
| | AAGACTTGGAGGGACGCCCTTGTGCCCCCTGAGCACGTGGGGAAGGCCCTGTCCTGGG | |
| | CCCTGCACCGCTCTGTGTGTTCATTGCACGAGTGTGGAAGCCTCAGAGACGCTGCATG | |
| | GTGTTCCCGGGCCATCCTGGCCAAGGTGGGAGAACGCAGCCTCCGTGAGGACCCGCGG | |
| | GATGCCCTCCCGCCCCCCCTCCTCCTTCTCCTCTCCTCTCAAGTGCAGACGACGCTGG | |
| | CCCACATCCTTGTCGGGCAGAGGGGCTGCCGACTGGCCCCTGGCCATGCCTCTTGCTG | |
| | GGAGCCGTGTGGCCTGGGCTTGGACCCACATCCACCTCTTGCTGGCTGCCTGCTCTTG | |
| | CCAGTGGCTGAGCCTGGCTTCTCCCATGACAGTGGCCGCAGACAGAGGGGACCCGTGC | |
| | CACCAGCCAAGGATGTGATCATTGGCAGGGATGAGATTGACGGGGACGGTGTCTGGGG | |
| | GCGAGGTGAGAGTTCTGCCAAAGCCGGAGCCAGGGTTGGACCCCCCGAGCTCGTTGGT | |
| | CCGCCCCTGCACGGAGAGTGGTGTGTTCACACCACGGCCCTGGTGGCCCAGCTCTGCC | |
| | CATCTTCATCCCAGGTGGCAGCTGCTGCCCAGCTCTCTGGCCAGTGCCTCTTAGATGG | |
| | TGTCACCACCTGCAGAAGAAAACAGAAGTGCTGA | |
| | ORF Start: ATG at 1 | ORF Stop: TGA at 1018 |
| | SEQ ID NO:150 | 339 aa MW at 36292.0 kD |

| | |
|---|---|
| NOV34a, | MAGQWEAGPSRRAGRIWAGEGKSACPYGNPVSLYTRSWLGGVFPVEERVREVRGAEQS |
| CG59301-01 Protein | EGFGSQPGWICFVALVAGWDGRVPVLFRRSRPTDAVLCPRRVLSEDPQLGRDPGRPLS |
| Sequence | KTWRDALVPPEHVGKALSWALHRSVCSLHECGSLRDAAWCSRGILAKVGERSLREDPR |
| | DALGGRLLVLVSSEVQTTLAHILVGQRCCRLAPGHGSCWEPCGLGLDPHPPLAGCLLL |
| | PVAEPGFSHDSGRRQRGPVPPGKDVIIGRDEIDGDGVWGRGESSAKACARVGPPELVG |
| | RPLEGEWCVHTTALVAQLCPSSSQVAAAAQLSGQCLLDGVTTCRRKQKC |

Further analysis of the NOV34a protein yielded the following properties shown in Table 34B.

TABLE 34B
Protein Sequence Properties NOV34a

| | |
|---|---|
| PSort | 0.4500 probability located in cytoplasm; 0.1626 probability |
| analysis: | located in lysosome (lumen); 0.1139 probability located in |
| | microbody (peroxisome); 0.1000 probability |
| | located in mitochondrial matrix space |
| SignalP | No Known Signal Sequence Indicated |
| analysis: | |

A search of the NOV34a protein against the Geneseq database, a proprietary database that contains sequences published in patents and patent publication, yielded several homologous proteins shown in Table 34C.

TABLE 34C
Geneseq Results for NOV34a

| | | | | |
|---|---|---|---|---|
| | | NOV34a | Identities/ | |
| | Protein/Organism/ | Residues/ | Similarities for | |
| Geneseq | Length [Patent #, | Match | the Matched | Expect |
| Identifier | Date] | Residues | Region | Value |

No Significant Matches Found

In a BLAST search of public sequence databases, the NOV34a protein was found to have homology to the proteins shown in the BLASTP data in Table 34D.

TABLE 34D
Public BLASTP Results for NOV34a

| | | | | |
|---|---|---|---|---|
| | | NOV34a | | |
| Protein | | Residues/ | Identities/ | |
| Accession | Protein/Organism/ | Match | Similarities for the | Expect |
| Number | Length | Residues | Matched Portion | Value |

No Significant Matches Found

PFam analysis indicates that the NOV34a protein contains the domains shown in the Table 34E.

TABLE 34E
Domain Analysis of NOV34a

| | NOV34a Match | Identities/Similarities | Expect |
|---|---|---|---|
| Pfam Domain | Region | for the Matched Region | Value |
| Androgen_recep: | 107 . . . 122 | 10/17 (59%) | 7.2 |
| domain 1 of 1 | | 11/17 (65%) | |
| EB: domain | 298 . . . 339 | 10/56 (18%) | 9.3 |
| 1 of 1 | | 26/56 (46%) | |

### Example 35

The NOV35 clone was analyzed, and the nucleotide and polypeptide sequences are shown in Table 35A.

TABLE 35A
NOV35 Sequence Analysis

| | SEQ ID NO:151 | 781 bp |
|---|---|---|
| NOV35a, | CAAACAGCCCCTACCCTCAATGATCTCCATACAAAGAGCTCCAAAGAGTGCCTCTCCC | |
| CG59525-01 DNA | CCTCCCTGCAGAGGGCGCATCCCCTGGCAGCGGTTCCTTCTCACAGCCTCAGTTTTAA | |
| Sequence | CTTTCTGGAACCCACCCACTACTGCCATGCTCACTGTTGAAGCCATGCTGTCCAATGC | |
| | CACAAAGGGGAAGGAGGTTCTTCTACTCACTCACAATCTGCCCCATGATCTTATTGGC | |
| | TATAACTGGTACAAAGGGGAAAGGGTGGAGGCCAACCACCATATTACAGGATATGTAA | |
| | TAGGAACTCTAATAACTACCCCAGGGCCTGCCCACAGCATTCAAGGGACAATATACCC | |
| | CAATCCATCCCTGCTGATTCAGAATGTCACCCAGGACACAGGATTCTACACCCTACAC | |
| | GCCATAAAGATAAACCCTGAGAAAGAAGAAGTATCTGGCCAGTTCCATGTATACGAAA | |
| | AAAATGCCCCAGGCCTTCCTGTGGGGGCCTTCACTGGCATCGTGACCAGGGTTCTGGT | |
| | CGGGGTGGCACCGGTGGCCACCCTGGCATGTTTCCTGCTCCTCGTCAGGACTGGAAGG | |
| | GCCAGTCGCCAGCATGACTTTAGAGAGCAGCTCCCAGGCCATGGTCCCTCAAACAACT | |
| | CCACCTACCCTACATCCCCTCTCTCCCCTGCCCAGGCCCCGCTACCCGACCCCAGGAC | |
| | AGCCGCTCCCATCTATGAGGAATTGCTAAACCATGACACAAACATTTACTGCTGGGTC | |
| | AACCACAAAGCAGATGTGCTTTCTTAG | |
| | ORF Start: ATG at 20 | ORF Stop: TAG at 779 |
| | SEQ ID NO:152 | 253 aa MW at 27528.2 kD |

| | |
|---|---|
| NOV35a, | MISIQRAPKSASPPPCRGRIPWQGFLLTASVLTFWNPPTTAMLTVEAMLSNATKGKEV |
| CG59525-01 Protein | LLLTHNLPHDLIGYNWYKGERVEANHHITGYVIGTLITTPGPAHSIQGTIYPNASLLI |
| Sequence | QNVTQDTGFYTLHAIKINPEKEEVSGQFHVYEKNAPGLPVGAFTGIVTRVLVGVAPVA |
| | TLACFLLLVRTGRASGQHDFREQLPGHGPSNNSTYPTSPLSPAQAPLPDPRTAAPIYE |
| | ELLNHDTNIYCWVNHKADVVS |

Further analysis of the NOV35a protein yielded the following properties shown in Table 35B.

TABLE 35B
Protein Sequence Properties NOV35a

| | |
|---|---|
| PSort | 0.8500 probability located in endoplasmic reticulum |
| analysis: | (membrane); 0.4400 probability located in plasma membrane; |
| | 0.1358 probability located in microbody (peroxisome); 0.1000 |
| | probability located in mitochondrial inner membrane |
| SignalP | Cleavage site between residues 42 and 43 |
| analysis: | |

A search of the NOV35a protein against the Geneseq database, a proprietary database that contains sequences published in patents and patent publication, yielded several homologous proteins shown in Table 35C.

TABLE 35C
Geneseq Results for NOV35a

| | | NOV35a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAR22042 | Carcino embryonic antigen-related | 8 . . . 253 | 166/252 (65%) | 7e−87 |
| | antigen encoded by clone W264 - | 3 . . . 252 | 188/252 (73%) | |
| | Homo sapiens, 252 aa. [JP04045788-A, | | | |
| | 14-FEB-1992] | | | |
| AAR22041 | Carcino embryonic antigen-related | 8 . . . 253 | 134/252 (53%) | 4e−68 |
| | antigen encoding clone W236 - Homo | 3 . . . 244 | 165/252 (65%) | |
| | sapiens, 244 aa. [JP04045788-A, | | | |
| | 14-FEB-1992] | | | |
| AAW83138 | FL-CEA protein - Homo sapiens, 526 | 12 . . . 155 | 97/145 (66%) | 2e−48 |
| | aa. [US5843761-A, 01-DEC-1998] | 5 . . . 145 | 107/145 (72%) | |
| AAW06873 | Carcinoembryonic antigen CEA-c - | 12 . . . 155 | 97/145 (66%) | 2e−48 |
| | Homo sapiens, 526 aa. [US5571710-A, | 5 . . . 145 | 107/145 (72%) | |
| | 05-NOV-1996] | | | |
| AAR65166 | Biliary glycoprotein - Homo sapiens, | 12 . . . 155 | 97/145 (66%) | 2e−48 |
| | 464 aa. [WO9506067-A1, | 5 . . . 145 | 107/145 (72%) | |
| | 02-MAR-1995] | | | |

In a BLAST search of public sequence databases, the NOV35a protein was found to have homology to the proteins shown in the BLASTP data in Table 35D.

TABLE 35D
Public BLASTP Results for NOV35a

| | | NOV35a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P40198 | Carcinoembryonic antigen-related cell | 8 . . . 253 | 167/252 (66%) | 7e−87 |
| | adhesion molecule 3 precursor | 3 . . . 252 | 189/252 (74%) | |
| | (Carcinoembryonic antigen CGM1) | | | |
| | (CD66d antigen) - Homo sapiens | | | |
| | (Human), 252 aa. | | | |
| O75871 | NONSPECIFIC CROSS-REACTING | 8 . . . 253 | 137/252 (54%) | 2e−69 |
| | ANTIGEN W236 - Homo sapiens | 3 . . . 244 | 168/252 (66%) | |
| | (Human), 244 aa. | | | |
| Q03715 | CGM7 NONSPECIFIC CROSS- | 8 . . . 253 | 134/252 (53%) | 2e−67 |
| | REACTING ANTIGEN (NCA) - Homo | 3 . . . 244 | 165/252 (65%) | |
| | sapiens (Human), 244 aa. | | | |
| S33324 | carcinoembryonic antigen - human, 212 | 12 . . . 187 | 121/177 (68%) | 7e−62 |
| | aa. | 5 . . . 181 | 135/177 (75%) | |
| Q13858 | BILIARY GLYCOPROTEIN - Homo | 12 . . . 199 | 120/193 (62%) | 1e−57 |
| | sapiens (Human), 252 aa. | 5 . . . 197 | 133/193 (68%) | |

PFam analysis indicates that the NOV35a protein contains the domains shown in the Table 35E.

TABLE 35E
Domain Analysis of NOV35a

| | | | |
|---|---|---|---|
| | NOV35a | Identities/Similarities | Expect |
| Pfam Domain | Match Region | for the Matched Region | Value |

No Significant Matches Found

### Example 36

The NOV36 clone was analyzed, and the nucleotide and polypeptide sequences are shown in Table 36A.

TABLE 36A
NOV36 Sequence Analysis

| | SEQ ID NO:153 | 4517 bp |
|---|---|---|
| NOV36a, | GTTTGAAAACACAAACACAATGGCAGGAAACAGCCTTGTTCTACCCATTGTTCTTTGG | |
| CG59484-01 DNA | GGTCGAAAAGCGCCCACACATTGCATCTCAGCCGTACTTTTAACAGATGATGGGCCCA | |
| Sequence | CGATCGTAACAGGATGTCACGACGGACAAATATGTCTCTGGGATCTTTCAGTAGAACT | |
| | GCAAGTGAATCCTCGAGCACTGTTGTTTGGTCATACAGCATCAATCACTTGTTTGTCT | |
| | AAAGCTTGTGCTTCCAGTGACAAACAGTATATTGTGAGTGCATCTGAAAGTGGGGAGA | |
| | TGTGCCTCTGGGATGTGAGTGATGGCAGATGTATTGAATTTACAAAATTAGCTTGCAC | |
| | ACATACTGGCATACAGTTCTACCAGTTCTCTGTTGGGAATCAGCGAGAAGGAAGGCTT | |
| | TTATGCCACGGACATTACCCTGAAATCCTTGTTGTGGATCCTACCAGCCTTGAAGTAT | |
| | TATACTCCTTAGTATCAAAGATATCACCAGACTGGATTAGCTCCATGAGTATTATTCG | |
| | ATCCCACCGAACACAAGAGGACACAGTGGTAGCACTCTCGGTGACTGGCATCCTGAAG | |
| | GTCTGGATTGTTACCTCGGAAATAAGTGACATGCAGGATACTGAGCCAATATTTCAGG | |
| | AGGAATCCAAACCAATTTATTGTCAGAATTGCCAAAGCATCTCTTTTTGTGCATTTAC | |
| | ACAAAGGTCACTTTTGGTTGTGTGTTCCAAATATTCGAGGGTATTCGATGCCGGAGAC | |
| | TATTCCTTGTTGTGTTCAGGTCCTAGTGAAAATGGACAGACATGGACCGGGGGGGACT | |
| | TTGTCTCATCAGATAAAGTCATCATTTGGACAGAAAATGGGCAAAGTTATATTTACAA | |
| | ACTACCTGCCAGTTGCCTTCCAGCTAGTGATTCATTCCGCAGTGATGTGGGGAAGGCA | |
| | GTTGAAAATTTAATTCCTCCTCTACAACATATCCTCTTGGATCGAAAAGATAAAGAGT | |
| | TGCTAATTTGTCCTCCTGTTACTCGGTTCTTCTATGGATGCAGAGAATATTTCCATAA | |
| | ACTGTTAATTCAGGGTGATTCTTCTGGAAGGTTGAATATTTGGAACATATCAGACACA | |
| | GCTGATAAACAGGGAAGTGAAGAAGGGCTGGCAATGACAACTTCTATTAGTTTGCAAG | |
| | AGGCATTTGATAAACTGAATCCTTGTCCTGCTGGAATTATAGATCAGCTGAGTGTGAT | |
| | TCCCAATAGTAATGAACCTCTTAAAGTAACTGCAAGTGTGTACATACCAGCACATGGA | |
| | CGACTTGTTTGTGGTCGTGAAGATGGAAGCATAGTTATTGTACCTGCCACACAGACGG | |
| | CCATAGTACAGCTGTTGCAAGGGGAACACATGCTCAGAAGAGGTTGGCCACCTCACAG | |
| | AACACTCCGTGGTCATCGGAACAAAGTCACATGTTTGCTATATCCTCATCAGGTCTCA | |
| | GCTCGGTATGATCAAAGATACCTGATATCTGGAGGTGTGGATTTTTCAGTCATAATTT | |
| | GGGACATATTTTCTGGAGAAATGAAACATATCTTCTGTGTTCATGGTGGTGAGATTAC | |
| | TCAACTTCTAGTTCCACCTGAAAACTGTAGTGCAAGAGTACAGCACTGCATCTGCTCT | |
| | GTAGCCAGTGACCACTCAGTAGGACTTCTAAGTTTGCGAGAGAAAAAATGCATAATGT | |
| | TGGCATCTCGTCACCTTTTTCCTATTCAAGTAATCAAATGGAGGCCTTCTGATGATTA | |
| | CCTGGTGGTGGGGTGTTCAGATGGTTCTGTGTACGTCTGGCAAATGGATACTGGTGCA | |
| | TTGGATCGTTGTGTGATGGGGATAACAGCAGTTGAGATTCTAAACGCTTGTGATGAAG | |
| | CTGTTCCTGCTGCTGTTGATTCACTTAGTCATCCAGCAGTCAACCTAAAACAAGCTAT | |
| | GACGAGACGTAGTCTTGCTGCTCTTAAAAATATGGCCCATCATAAGCTACAAACCCTT | |
| | GCAACTAACCTCTTGGCTTCTGAGGCATCTGACAAGGGAAATTTACCTAAATATTCTC | |
| | ATAACTCCCTGATGGTTCAAGCAATAAAGACAAACCTAACAGACCCGGACATACATGT | |
| | GCTATTCTTTGATGTGGAAGCGTTGATTATTCAACTCCTGACTGAAGAAGCCTCTAGG | |
| | CCGAATACTGCTCTTATTTCCCCAGAGAATTTGCAAAAAGCATCTGCCAGTTCAGACA | |
| | AAGGGGGCTCTTTTTTAACTGGAAAACGAGCAGCAGTTCTCTTCCAACAAGTGAAAGA | |
| | AACGATCAAAGAGAACATCAAGGAACACCTCCTTGATGATGAACAGGAGGATGAGGAG | |
| | ATAATGAGGCAGAGAAGGGAAGAAAGTGATCCTGAATATCGGTCCAGCAAATCAAAGC | |
| | CATTGACCCTATTAGAATATAATTTAACTATGGACACTGCAAAGCTGTTTATGTCCTG | |
| | CCTTCACGCCTGGGGTTTGAATGAAGTACTGGATGAAGTTTGCCTGGATCGCCTTGGA | |
| | ATGCTGAAACCCCACTGCACCGTATCGTTTGGCCTCTTGTCAAGAGGAGGCCATATGT | |
| | CACTGATGCTGCCGGGTTATAATCAGCCTGCTTGTAAACTGTCACATGGGAAAACAGA | |
| | AGTAGGAAGGAAGCTGCCAGCGTCTGAGGGAGTAGGAAAGGCAACTTACGCAGTGTCC | |
| | CGTGCCGTCACCACACAGCATCTCCTGTCTATCATTTCTTTGGCAAATACTTTAATGA | |
| | GTATGACCAATGCAACTTTTATTGGTGATCATATGAAGAAGGGTCCTACCAGGCCACC | |
| | TAGACCAAGCACCCCAGACCTTTCTAAGGCAAGGGGTTCCCCTCCAACTTCCAGTAAT | |
| | ATTGTGCAAGGACAGATTAAACAAGTTGCTGCACCTGTCGTTTCCGCTCGGTCTGATG | |
| | CTGATCACTCTGGCTCTGACCCTCCTTCTGCTCCTGCTTTACATACCTGTTTCTTAGT | |
| | AAATGAAGGTTCGAGTCAGTTAGCTGCTATGCACTGTGTTATGCTGCCAGACCTACTG | |
| | GGATTGGATAAATTTAGCCCTCCCCTTCTGCAGATGCTCGCCCGAAGATGGCAAGATC | |
| | GATGCTTGGAGGTAAGAGAAGCCGCGCAGGCCCTGCTTCTGGCGGAACTGAGAAGAAT | |
| | TGAGCAGGCAGGCAGGAAGGAAGCCATTGATGCCTCGGCTCCTTACTTACCTCAGTAC | |
| | ATAGACCACGTCATATCACGTGGAGTCACATCAGAAGCCGCGCAGACTATCACCACGG | |
| | CTCCTGATGCCTCAGGGCCTGAAGCAAAAGTCCAGGAGGAAGAGCATGACCTTGTTGA | |
| | CGATGACATCACCACTGGTTGCTTATCAAGTGTCCCACAAATGAAAAAAATTTCTACA | |
| | TCTTACGAGGAAAGACGGAAGCAAGCTACCGCTATTGTTTTACTTGCAGTAATAGGAG | |
| | CTGAATTTGGTGCTGAAATTGAACCTCCTAAACTATTGACCAGACCTCGAAGCTCTAG | |
| | CCAAATTCCTGAGGGATTCGGGTTGACTAGTGGTGGATCCAACTACTCGCTGGCCAGA | |
| | CATACTTCCAAGGCACTGACGTTTCTTCTGCTACAGCCTCCAAGCCCCAAACTTCCTC | |
| | CACACAGCACTATCCGAAGAACAGCCATTGATCTGATTGGACGTGGGTTCACTGTTTG | |
| | GGAGCCTTACATGGATGTGTCCGCTGTTCTGATGGGGCTTCTCGAACTTTCTGCCGAT | |
| | GCCGAGAAACAACTTGCCAACATCACAATGGGGTTGCCTCTGAGCCCAGCAGCTGACT | |
| | CGGCCCGCTCTGCGAGCCATGCCCTCTCGCTCATTGCCACCCCCAGACCACCCGCCTT | |
| | CATCACCACCATAGCCAAAGAGGTACACAGACATACGGCTCTTGCAGCAAATACCCAA | |
| | TCACAGCAGAATATGCACACAACAACTCTTGCACGAGCTAAAGGGGAAATTTTGAGAG | |
| | TCATTGAAATTCTTATTGAAAAGATGCCCACAGATGTTGTGGATCTTCTCGTGGAGGT | |
| | TATGGACATCATTATGTACTGCCTTGAACGATCTTTAGTTAAAAAGAAAGGTCTTCAA | |
| | GAATGTTTCCCAGCCATCTGCAGGTTCTACATGGTCAGCTATTATGAGCGGAATCACA | |
| | GAATAGCAGTTCGAGCTCGCCATGGTTCAGTGGCCCTGTACGACATCCGGACTGGAAA | |
| | ATGTCAGACAATCCATGGACACAAGGGACCAATCACTGCAGTGCCTTTTGCTCCTGAT | |
| | GGAAGATATCTTGCCACCTACTCAAACACTGACAGCCACATTTCTTTTTGGCAGATGA | |
| | ACACGTCACTGCTGGGAACCATCGGCATGCTGAACTCGGCACCTCAGCTGCGCTGCAT | |
| | TAAAACCTACCAGGTGCCCCCTGTGCAGCCCGCGTCCCCCGGCTCCCACAATGCCCTC | |
| | AAGCTGGCCCGGCTCATCTGGACTTCCAACCGCAACGTCATCCTCATGGCCCATGACG | |
| | GGAAGGAGCACCGCTTCATGGTCTAATGCTGCTGCCTGCCGCCGTGACTGC | |
| | ORF Start: ATG at 20 | ORF Stop: TAA at 4490 |
| | SEQ ID NO:154 | 1490 aa MW at 163853.2 kD |

| | |
|---|---|
| NOV36a, | MAGNSLVLPIVLWGRKAPTHCISAVLLTDDGATIVTCCHDGQICLWDLSVELQVNPRA |
| CG59484-01 Protein | LLFGHTASITCLSKACASSDKQYIVSASESGEMCLWDVSDGRCIEFTKLACTHTGIQF |
| Sequence | YQFSVGNQREGRLLCHGHYPEILVVDATSLEVLYSLVSKISPDWISSMSIIRSHRTQE |
| | DTVVALSVTGILKVWIVTSEISDMQDTEPIFEEESKPIYCQNCQSISFCAFTQRSLLV |
| | VCSKYWRVFDAGDYSLLCSGPSENGQTWTGGDFVSSDKVIIWTENGQSYIYKLPASCL |
| | PASDSFRSDVGKAVENLIPPVQHILLDRKDKELLICPPVTRFFYGCREYFHKLLIQGD |
| | SSGRLNIWNISDTADKQGSEEGLAMTTSISLQEAFDKLNPCPAGIIDQLSVIPNSNEP |
| | LKVTASVYIPAHGRLVCGREDGSIVIVPATQTAIVQLLQGEHMLRRGWPPHRTLRGHR |
| | NKVTCLLYPHQVSARYDQRYLISGGVDFSVIIWDIFSGEMKHIFCVHGGEITQLLVPP |
| | ENCSARVQHCICSVASDHSVGLLSLREKKCIMLASRHLFPIQVIKWRPSDDYLVVGCS |
| | DGSVYVWQMDTGALDRCVMGITAVEILNACDEAVPAAVDSLSHPAVNLKQAMTRRSLA |
| | ALKNMAHHKLQTLATNLLASEASDKGNLPKYSHNSLMVQAIKTNLTDPDIHVLFFDVE |
| | ALlIQLLTEEASRPNTALISPENLQKASGSSDKGGSFLTGKPAAVLFQQVKETIKENI |
| | KEHLLDDEEEDEEIMRQRREESDPEYRSSKSKPLTLLEYNLTMDTAKLFMSCLHAWGL |
| | NEVLDEVCLDRLGMLKPHCTVSFGLLSRGGHMSLMLPGYNQPACKLSHGKTEVGRKLP |
| | ASEGVGKGTYGVSRAVTTQHLLSIISLANTLMSMTNATFIGDHMKKGPTRPPRPSTPD |
| | LSKARGSPPTSSNIVQGQIKQVAAPVVSARSDADHSGSDPPSAPALHTCFLVNEGWSQ |
| | LAAMHCVMLPDLLGLDKFRPPLLEMLARRWQDRCLEVREAAQALLLAELRRIEQAGRK |
| | EAIDAWAPYLPQYIDHVISRGVTSEAAQTITTAPDASGPEAKVQEEEHDLVDDDITTG |
| | CLSSVPQMKKISTSYEERRKQATAIVLLGVIGAEFGAEIEPPKLLTRPRSSSQIPEGF |
| | GLTSGGSNYSLARHTCKALTFLLLQPPSPKLPPHSTIRRTAIDLIGRGFTVWEPYMDV |
| | SAVLMGLLELCADAEKQLANITMGLPLSPAALSARSARHALSLIATARPPAFITTIAK |
| | EVHRHTALAANTQSQQNMHTTTLARAKGEILRVIEILIEKMPTDVVDLLVEVMDIIMY |
| | CLEGSLVKKKGLQECFPAICRFYMVSYYERNHRIAVGARHGSVALYDIRTGKCQTIHG |
| | HKGPITAVAFAPDGRYLATYSNTDSHISFWQMNTSLLGSIGMLNSAPQLRCIKTYQVP |
| | PVQPASPGSHNALKLARLIWTSNRNVILMAHDGKEHRFMV |

Further analysis of the NOV36a protein yielded the following properties shown in Table 36B.

TABLE 36B
Protein Sequence Properties NOV36a

| | |
|---|---|
| PSort | 0.8110 probability located in plasma membrane; 0.6400 |
| analysis: | probability located in endoplasmic reticulum (membrane); |
| | 0.3700 probability located in Golgi body; |
| | 0.1000 probability located in endoplasmic reticulum (lumen) |
| SignalP | Cleavage site between residues 22 and 23 |
| analysis: | |

A search of the NOV36a protein against the Geneseq database, a proprietary database that contains sequences published in patents and patent publication, yielded several homologous proteins shown in Table 36C.

TABLE 36C
Geneseq Results for NOV36a

| | | NOV36a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAE10802 | Human TGF-beta Resistance | 1 . . . 1490 | 1489/1490 (99%) | 0.0 |
| | Associated Gene (TRAG) protein - | 1 . . . 1490 | 1489/1490 (99%) | |
| | Homo sapiens, 1490 aa. | | | |
| | [WO200166739-A1, 13-SEP-2001] | | | |
| AAE10803 | Mouse TGF-beta Resistance | 1 . . . 1490 | 1415/1490 (94%) | 0.0 |
| | Associated Gene (TRAG) protein - | 1 . . . 1489 | 1450/1490 (96%) | |
| | Mus sp, 1489 aa. [WO200166739-A1, | | | |
| | 13-SEP-2001] | | | |
| AAE10801 | Rat TGF-beta Resistance Associated | 1 . . . 1490 | 1408/1490 (94%) | 0.0 |
| | Gene (TRAG) protein - Rattus sp, | 1 . . . 1488 | 1446/1490 (96%) | |
| | 1488 aa. [WO200166739-A1, | | | |
| | 13-SEP-2001] | | | |
| AAM40548 | Human polypeptide SEQ ID NO 5479 - | 454 . . . 594 | 38/146 (26%) | 2e−07 |
| | Homo sapiens, 277 aa. | 48 . . . 181 | 63/146 (43%) | |
| | [WO200153312-A1, 26-JUL-2001] | | | |
| AAM40547 | Homo sapiens, 277 aa. | 48 . . . 181 | 63/146 (43%) | 2e−07 |
| | [WO200153312-A1, 26-JUL-2001] | | | |

In a BLAST search of public sequence databases, the NOV36a protein was found to have homology to the proteins shown in the BLASTP data in Table 36D.

TABLE 36D
Public BLASTP Results for NOV36a

| | | NOV36a | | |
|---|---|---|---|---|
| Protein | | Residues/ | Identities/ | |
| Accession | | Match | Similarities for the | Expect |
| Number | Protein/Organism/Length | Residues | Matched Portion | Value |
| CAC88575 | SEQUENCE 3 FROM PATENT | 1 . . . 1490 | 1489/1490 (99%) | 0.0 |
| | WO0166739 - Homo sapiens | 1 . . . 1490 | 1489/1490 (99%) | |
| | (Human), 1490 aa. | | | |
| Q96PS7 | TGF-BETA RESISTANCE- | 1 . . . 1490 | 1456/1490 (97%) | 0.0 |
| | ASSOCIATED PROTEIN TRAG - | 1 . . . 1457 | 1456/1490 (97%) | |
| | Homo sapiens (Human), 1457 aa | | | |
| | (fragment). | | | |
| CAC88576 | SEQUENCES 5 FROM PATENT | 1 . . . 1490 | 1415/1490 (94%) | 0.0 |
| | WO0166739 - unidentified, 1489 aa. | 1 . . . 1489 | 1450/1490 (96%) | |
| Q92019 | TGF-BETA RESISTANCE- | 1 . . . 1490 | 1414/1490 (94%) | 0.0 |
| | ASSOCIATED PROTEIN TRAG - | 1 . . . 1489 | 1449/1490 (96%) | |
| | Mus musculus (Mouse), 1489 aa | | | |
| | (fragment). | | | |
| Q9ERH3 | TGF-BETA RESISTANCE- | 1 . . . 1490 | 1408/1490 (94%) | 0.0 |
| | ASSOCIATED PROTEIN - Rattus | 1 . . . 1488 | 1446/1490 (96%) | |
| | norvegicus (Rat), 1488 aa. | | | |

PFam analysis indicates that the NOV36a protein contains the domains shown in Table 36E.

TABLE 36E
Domain Analysis of NOV36a

| | | Identities/ | |
|---|---|---|---|
| | | Similarities | |
| | NOV36a Match | for the Matched | Expect |
| Pfam Domain | Region | Region | Value |
| WD40: domain 1 of 8 | 11 . . . 47 | 9/37 (24%) | 0.022 |
| | | 29/37 (78%) | |
| WD40: domain 2 of 8 | 56 . . . 95 | 10/40 (25%) | 14 |
| | | 30/40 (75%) | |
| WD40: domain 3 of 8 | 151 . . . 190 | 5/41 (12%) | 1.5e+03 |
| | | 27/41 (66%) | |
| WD40: domain 4 of 8 | 209 . . . 242 | 6/37 (16%) | 5.7e+02 |
| | | 23/37 (62%) | |
| HSF_DNA-bind: | 267 . . . 282 | 7/16 (44%) | 3.1 |
| domain 1 of 1 | | 14/16 (88%) | |
| WD40: domain 5 of 8 | 456 . . . 498 | 15/43 (35%) | 0.0048 |
| | | 30/43 (70%) | |
| WD40: domain 6 of 8 | 504 . . . 546 | 6/43 (14%) | 2.7e+02 |
| | | 27/43 (63%) | |
| WD40: domain 7 of 8 | 552 . . . 588 | 8/37 (22%) | 0.22 |
| | | 26/37 (70%) | |
| WD40: domain 8 of 8 | 1386 . . . 1423 | 11/38 (29%) | 4 |
| | | 29/38 (76%) | |

### Example 37

The NOV37 clone was analyzed, and the nucleotide and polypeptide sequences are shown in Table 37A.

TABLE 37A
NOV37 Sequence Analysis

| | SEQ ID NO:155 | 848 bp |
|---|---|---|
| NOV37a, | GCCTCGCTCGGGCGCCCAGTGGTCCTGCCGCCTGGTCTCACCTCGCCATGGTTCGTCT | |
| CG57245-02 DNA | GCCTCTGCAGTGCGTCCTCTGGGGCTGCTTGCTGACCGCTGTCCATCCAGAACCACCC | |
| Sequence | ACTGCATGCAGAGAAAAACAGTACCTAATAAACAGTCAGTGCTGTTCTTTGTCCCAGC | |
| | CAGGACAGAAACTGGTGAGTGACTGCACAGAGTTCACTGAAACGGPATGCCTTCCTTG | |
| | CGGTGAAAGCGAATTCCTAGACACCTGGAACAGAGAGACACACTGCCACCAGCACAAA | |
| | TACTGCGACCCCAACCTAGGGCTTCGGGTCCAGCAGAAGGGCACCTCAGAAACAGACA | |
| | CCATCTGCACCTGTGAAGAAGGCTGGCACTGTACGAGTGAGGCCTGTGAGAGCTGTGT | |
| | CCTGCACCGCTCATGCTCGCCCGGCTTTGGGGTCAAGCAGATTGGTCCCCAGGATCGG | |
| | CTGAGAGCCCTGGTCGTGATCCCCATCATCTTCGGGATCCTGTTTGCCATCCTCTTGG | |
| | TGCTGGTCTTTATCAAAAAGGTGGCCAAGAAGCCAACCAATAAGGCCCCCCACCCCAA | |
| | GCAGGAACCCCAGGAGATCAATTTTCCCGACGATCTTCCTGGCTCCAACACTGCTGCT | |
| | CCAGTGCAGGACACTTTACATGGATGCCAACCGGTCACCCAGGAGGATGGCAAAGAGA | |
| | GTCGCATCTCAGTGCAGGAGAGACAGTGAGGCTGCACCCACCCAGGAGTGTGGCCACG | |
| | TGGCCAAACAGGCAGTTGGCCAGAGAGCCTGGTGCTGCTGCTGCAGGGGTGCAGGCAG | |
| | AAGCGGGGAGCTATGCCCAGTCAGTGCCAGCCCCTC | |
| | ORF Start: ATG at 48 | ORF Stop: TGA at 723 |
| | SEQ ID NO:156 | 225 aa MW at 25098.5 kD |
| NOV37a, | MVRLPLQCVLWGCLLTAVHPEPPTACREKQYLINSQCCSLCQPGQKLVSDCTEFTETE | |
| CG57245-02 Protein | CLPCCESEFLDTWNRETHCHQHKYCDPNLGLRVQQKGTSETDTICTCEEGWHCTSEAC | |
| Sequence | ESCVLHRSCSPGFGVKQIGPQDRLRALVVIPIIFGILFAILLVLVFIKKVAKKPTNKA | |
| | PHPKQEPQEINFPDDLPGSNTAAPVQETLHGCQPVTQEDCKESRISVQERQ | |
| | SEQ ID NO:157 | 1016 bp |
| NOV37b, | GCCTCGCTCGGGCGCCCAGTGGTCCTGCCGCCTGGTCTCACCTCGCCATGGTTCGTCT | |
| CG57245-04 DNA | GCCTCTGCAGTGCGTCCTCTGGGGCTGCTTGCTGACCGCTGTCCATCCAGAACCACCC | |
| Sequence | ACTGCATGCAGAGAAAAACAGTACCTAATAAACAGTCAGTGCTGTTCTTTGTGCCAGC | |
| | CAGGACACAAACTGGTGAGTGACTGCACAGAGTTCACTGAAGCGGAATGCCTTCCTTG | |
| | CGGTGAAACCGAATTCCTAGACACCTGGAACAGAGAGACACACTGCCACCAGCACAAA | |
| | TACTGCGACCCCAACCTAGGGCTTCGGGTCCAGCAGAAGGGCACCTCAGAAACAGACA | |
| | CCATCTGCACCTGTGAAGAAGGCTGGCACTGTACGAGTGAGGCCTGTGAGAGCTGTGT | |
| | CCTGCACCGCTCATGCTCGCCCGGCTTTGGGGTCAAGCAGATTGCTACAGGGGTTTCT | |
| | GATACCATCTGCGAGCCCTGCCCAGTCGGCTTCTTCTCCAATGTGTCATCTGCTTTCG | |
| | AAAAATGCCACCCTTGCACAAGCTGTGAGACCAAACACCTGGTTGTGCAACAGGCAGG | |
| | CACAAACAAGACTGATGTTGTCCGTGGTCCCCAGGATCGGCTGAGAGCCCTGGTGGTG | |
| | ATCCCCATCATCTTCGGGATCCTGTTTGCCATCCTCCTGGTGCTGGTCTTTATCAGTG | |
| | AGTCCTCAGAAAAGGTGGCCAAGAAGCCAACCAATAAGGCCCCCCACCCCAAGCAGGA | |
| | ACCCCAGGAGATCAATTTTCCCGACGATCTTCCTGGCTCCAACACTGCTGCTCCAGTG | |
| | CAGGAGACTTTACATGGATGCCAACCGGTCACCCAGGAGGATGGCAAAGAGAGTCGCA | |
| | TCTCAGTGCAGGAGAGACAGTGAGGCTGCACCCACCCAGGAGTGTGGCCACGTGGGCA | |
| | AACAGGCAGTTGGCCAGAGAGCCTGGTGCTGCTGCTGCAGGGGTGCAGGCAGAAGCCG | |
| | GGAGCTATGCCCAGTCAGTGCCAGCCCCTC | |
| | ORF Start: ATG at 48 | ORF Stop: TGA at 891 |
| | SEQ ID NO:158 | 1281 aa MW at 31033.0 kD |
| NOV37b, | MVRLPLQCVLWGCLLTAVHPEPPTACREKQYLINSQCCSLCQPGQKLVSDCTEFTEAE | |
| CG57245-04 Protein | CLPCGESEFLDTWNRETHCHQHKYCDPNLGLRVQQKGTSETDTICTCEEGWHCTSEAC | |
| Sequence | ESCVLHRSCSPGFGVKQIATGVSDTICEPCPVGFFSNVSSAFEKCHPWTSCETKDLVV | |
| | QQAGTNKTDVVRCPQDRLRALVVIPIIFGILFAILLVLVFISESSEKVAKKPTNKAPH | |
| | PKQEPQEINFPDDLPGSNTAAPVQETLHGCQPVTQEDGKESRISVQERQ | |
| | SEQ ID NO:159 | 375 bp |
| NOV37c, | AGATCTGAACCACCCACTGCATGCAGAGAAAAACAGTACCTAATAAACAGTCAGTCCT | |
| 174308232 DNA | GTTCTTTGTGCCAGCCAGGACAGAAACTGGTGAGTGACTGCACAGAGTTCACTGAAAC | |
| Sequence | GCAATGCCTTCCTTGCGGTGAAAGCGAATTCCTAGACACCTGGAACAGAGAGACACAC | |
| | TGCCACCAGCACAAATACTGCGACCCCAACCTAGGGCTTCGGGTCCAGCAGAAGGGCA | |
| | CCTCAGAAACAGACACCATCTGCACCTGTGAAGAAGGCTGGCACTGTACGAGTGAGGC | |
| | CTGTGAGAGCTGTGTCCTGCACCGCTCATGCTCGCCCGGCTTTGGGGTCAAGCAGATT | |
| | GGTCCCCAGGATCGGCTGAGACTCGAG | |
| | ORF Start: AGA at 1 | ORF Stop: at 376 |
| | SEQ ID NO:160 | 125 aa MW at 14132.6 kD |

| | |
|---|---|
| NOV37c, | RSEPPTACREKQYLINSQCCSLCQPGQKLVSDCTEFTETECLPCGESEFLDTWNRETH |
| 174308232 Protein | CHQHKYCDPNLGLRVQQKGTSETDTICTCEEGWHCTSEACESCVLHRSCSPGFGVKQI |
| Sequence | CPQDRLRLE |

Sequence comparison of the above protein sequences yields the following sequence relationships shown in Table 37B.

TABLE 37B
Comparison of NOV37a against NOV37b through NOV37c.

| | NOV37a Residues/ | Identities/Similarities |
|---|---|---|
| Protein Sequence | Match Residues | for the Matched Region |
| NOV37b | 1 . . . 225 | 204/281 (72%) |
| | 1 . . . 281 | 205/281 (72%) |
| NOV37c | 21 . . . 141 | 121/121 (100%) |
| | 3 . . . 123 | 121/121 (100%) |

Further analysis of the NOV37a protein yielded the following properties shown in Table 37C.

TABLE 37C
Protein Sequence Properties NOV37a

| | |
|---|---|
| PSort | 0.4600 probability located in plasma membrane; |
| analysis: | 0.1000 probability located in |
| | endoplasmic reticulum (membrane); |
| | 0.1000 probability located in endoplasmic |
| | reticulum (lumen); 0.1000 probability located in outside |
| SignalP | Cleavage site between residues 21 and 22 |
| analysis: | |

A search of the NOV37a protein against the Geneseq database, a proprietary database that contains sequences published in patents and patent publication, yielded several homologous proteins shown in Table 37D.

TABLE 37D
Geneseq Results for NOV37a

| | | NOV37a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAB20170 | Human CD40 receptor splice variant - | 1 . . . 225 | 225/246 (91%) | e−134 |
| | Homo sapiens, 246 aa. [WO200105967- | 1 . . . 246 | 225/246 (91%) | |
| | A1, 25-JAN-2001] | | | |
| AAB84892 | Human CD40 antigen - Homo sapiens, | 1 . . . 225 | 225/277 (81%) | e−130 |
| | 277 aa. [WO200130974-A2, 03-MAY-2001] | 1 . . . 277 | 225/277 (81%) | |
| AAB37807 | Human CD40 - Homo sapiens, 277 aa. | 1 . . . 225 | 225/277 (81%) | e−130 |
| | [WO200066155-A1, 09-NOV-2000] | 1 . . . 277 | 225/277 (81%) | |
| AAB50520 | Human tumour necrosis factor receptor | 1 . . . 225 | 225/277 (81%) | e−130 |
| | CD40 protein SEQ ID NO: 10 - Homo | 1 . . . 277 | 225/277 (81%) | |
| | sapiens, 277 aa. [WO200071150-A1, | | | |
| | 30-NOV-2000] | | | |
| AAY52701 | Human CD40 protein - Homo sapiens, | 1 . . . 225 | 225/277 (81%) | e−130 |
| | 277 aa. [WO9953101-A1, 21-OCT-1999] | 1 . . . 277 | 225/277 (81%) | |

In a BLAST search of public sequence databases, the NOV37a protein was found to have homology to the proteins shown in the BLASTP data in Table 37E.

TABLE 37E
Public BLASTP Results for NOV37a

| | | NOV37a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P25942 | Tumor necrosis factor receptor | 1 . . . 225 | 225/277 (81%) | e−130 |
| | superfamily member 5 precursor | 1 . . . 277 | 225/277 (81%) | |
| | (CD40L receptor) (B-cell surface | | | |
| | antigen CD40) (Bp50) (CDw40) - Homo | | | |
| | sapiens (Human), 277 aa. | | | |
| Q9BYU0 | CD40 TYPE II ISOFORM - Homo | 1 . . . 134 | 134/134 (100%) | 1e−82 |
| | sapiens (Human), 203 aa. | 1 . . . 134 | 134/134 (100%) | |
| A46476 | B cell-associated surface molecule | 1 . . . 225 | 137/281 (48%) | 3e−73 |
| | CD40, long splice form - mouse, 305 aa. | 1 . . . 278 | 168/281 (59%) | |
| P27512 | Tumor necrosis factor receptor | 1 . . . 225 | 137/281 (48%) | 3e−73 |
| | superfamily member 5 precursor | 1 . . . 278 | 168/281 (59%) | |
| | (CD40L receptor) (B-cell surface | | | |
| | antigen CD40) (BP50) (CDw40) - Mus | | | |
| | musculus (Mouse), 289 aa. | | | |
| Q99NE0 | CD40 TYPE V ISOFORM - Mus | 1 . . . 225 | 130/252 (51%) | 7e−67 |
| | musculus (Mouse), 260 aa. | 1 . . . 249 | 157/252 (61%) | |

PFam analysis indicates that the NOV37a protein contains the domains shown in the Table 37F.

TABLE 37F
Domain Analysis of NOV37a

| | | Identities/ | |
|---|---|---|---|
| | | Similarities | |
| | NOV37a | for the | Expect |
| Pfam Domain | Match Region | Matched Region | Value |
| TNFR_c6: domain | 26 . . . 59 | 19/42 (45%) | 2.7e−09 |
| 1 of 2 | | 30/42 (71%) | |
| EB: domain | 26 . . . 77 | 10/60 (17%) | 8.3 |
| 1 of 1 | | 33/60 (55%) | |
| TNFR_c6: domain | 62 . . . 103 | 11/44 (25%) | 0.9 |
| 2 of 2 | | 29/44 (66%) | |
| ATP-synt_B: domain | 148 . . . 174 | 5/27 (19%) | 6.8 |
| 1 of 1 | | 23/27 (85%) | |

### Example 38

The NOV38 clone was analyzed, and the nucleotide and polypeptide sequences are shown in Table 38A.

TABLE 38A
NOV38 Sequence Analysis

| | SEQ ID NO:161 | 1080 bp |
|---|---|---|
| NOV38a, | GATTTAATTGGCATTATTGTGTTCCCGACCATGACTGCAGAGACCATGGAGCTGAAGT | |
| CG59454-01 DNA | GGGTGAGTTTCCTGGCCTTCCTTCTGCTCAACTTTCGTGTCTGCCTCCTTTTGCTTCA | |
| Sequence | GCTGCTCATGCCTCACTCAGCTCAGTTTTCTCTCCTTGGACCCTCTGGGCCCATCCTG | |
| | GCCATGGTGGGTGAAGACGCTGATCTGCCCTGTCACCTGTTCCCGACCATGAGTGCAG | |
| | AGACCATGGAGCTGAAGTGGGTAAGTTCCAGCCTAAGGCAGGTGGTGAACGTGTATGC | |
| | AGATGGAAZGGAAGTGGAAGACAGGCAGAGTGCACCGTATCGAGGGAGAACTTCGATT | |
| | CTGCGGGATGGCATCACTGCGGGGAAGGCTGCTCTCCGAATACACAACGTCACAGCCT | |
| | CTGACAGTGGAAAGTACTTGTCTTATTTCCAAGATGGTGACTTCTATCAAAAACCCCT | |
| | GGTGGAGCTGAAGGTTGCAGCACTGGGTTCTAATCTTCACGTCGAAGTGAAGGGTTAT | |
| | GAAGATGGAGGCATCCATCTGCAGTGCAGGTCCACCCCCTGGTACCCCCAACCCCAAA | |
| | TACAGTGGGGCAACGCCAAGGGAGAGAACATCCCAGCTGTGGAAGCACCTGTGGTTGC | |
| | AGATGGAGTGGGCCTATATGAAGTAGCAGCATCTGTGATCATGAAAAGCCGCTCCGGG | |
| | GAAGGTGTATCCTGCATCATCAGAAATTCCCTCCTCGGCCTGGAAAAGACAGCCAGCA | |
| | TTTCCATCGCAGACCCCTTCTTCAGGAGCGCCCAGCCCTGGATCGCAGCCCTGGCAGG | |
| | GACCCTGCCTATCTTGCTGCTGCTTCTCGCCGGAGCCAGTTACTTCTTGTCGAGACAA | |
| | CAGAAGGAAATAACTGCTCTGTCCAGTGAGATAGAAAGTGAGCAAGAGATGAAAGAAA | |
| | TGGGATATGCTGCAACAGAGCGGGAAATAAGCCTAAGAGAGAGCCTCCAGGAGGAACT | |
| | CAAGAGGAAAAAAATCCAGTACTTGACTCGTGGAGAGGAGTCTTCGTCCGATACCAAT | |
| | AAGTCAGCCTGATGCTCTAATGGAAAAATGGCCCTC | |
| | ORF Start: ATG at 31 | ORF Stop: TGA at 1054 |
| | SEQ ID NO:162 | 341 aa MW at 37417.4 kD |
| NOV38a, | MSAETMELKWVSFLAFLLLNFRVCLLLLQLLMPHSAQFSVLGPSGPILAMVGEDADLP | |
| CG59454-01 Protein | CHLFPTMSAETMELKWVSSSLRQVVNVYADCKEVEDRQSAPYRGRTSILRDGITAGKA | |
| Sequence | ALRIHNVTASDSGKYLCYFQDGDFYEKALVELKVAALGSNLHVEVKGYEDGGIHLECR | |
| | STGWYPQPQIQWGNAKGENIPAVEAPVVADGVGLYEVAASVIMKSCSGEGVSCIIRNS | |
| | LLCLEKTASISIADPFFRSAQPWIAALAGTLPILLLLLAGASYFLWRQQKEITALSSE | |
| | IESEQEMKEMGYAATEREISLRESLQEELKRKKIQYLTRGEESSSDTNKSA | |
| | SEQ ID NO:163 | 935 bp |
| NOV38b, | ATTTGCTTTCTCTTTTTCCTTTCTTCCGGATGAGAGGCTAAGCCATAATAGAAAGAAT | |
| CG 59454-03 DNA | GGAGAATTATTGATTGACCGTCTTTATTCTGTGGGCTCTCATTCTCCAATGGGAATAC | |
| Sequence | CAAGCCATGGTTTTCCATACTGGAACCCAAAGGTAAAGACACTCAAGGACAGACATTT | |
| | TTGGCAGAGCATAGATGAAAATGGCAAGTTCCCTCGCTTTCCTTCTGCTCAACTTTCA | |
| | TGTCTCCCTCCTCTTGGTCCAGCTGCTCACTCCTTGCTCAGCTCAGTTTTCTGTGCTT | |
| | GGACCCTCTGGGCCCATCCTGGCCATGGTGGGTGAAGACGCTGATCTGCCCTGTCACC | |
| | TGTTCCCGACCATCAGTGCAGAGACCATCGAGCTGAAGTGGGTAAGTTCCAGCCTAAG | |
| | GCAGGTGGTGAATGTGTATGCAGATGGAAAGGAAGTGGAAGACAGGCAGACTGCACCG | |
| | TATCGAGGGAGAACTTCGATTCTGCGGGATGGCATCACTGCAGGGAAGGCTGCTCTCC | |
| | GAATACACAACGTCACAGCCTCTGACAGTGGAAAGTACTTGTGTTATTTCCAAGATGG | |
| | TGACTTCTATGAAAAAGCCCTGGTGGAGCTGAAGGTTGCAGACCCCTTCTTCAGGAGC | |
| | GCCCAGCCCTGGATCGCAGCCCTGGCAGGGACCCTGCCTATCTTGCTGCTGCTTCTCG | |
| | CCGGAGCCAGTTACTTCTTGTGGAGACAACACAAGGAAATAACTGCTCTGTCCAGTGA | |
| | GATAGAAAGTGAGCAAGAGATGAAAGAAATGGGATATGCTGCAACAGAGCGGGAAATA | |
| | AGCCTAAGAGAGACCCTCCAGGAGGAACTCAACAGGAAAAAAATCCAGTACTTGACTC | |
| | GTGGAGAGGAGTCTTCGTCCGATACCAATAAGTCAGCCTGATGCTCTAATGGAAAAAT | |
| | GGCCCTC | |
| | ORF Start: ATG at 189 | ORF Stop: TGA at 909 |
| | SEQ ID NO:164 | 240 aa MW at 26651.2 kD |
| NOV38b, | MKMASSLAFLLLNFHVSLLLVQLLTPCSAQFSVLGPSGPILAIVIVGEDADLPCHLFPTM | |
| CG59454-03 Protein | SAETMELKWVSSSLRQVVNVYADGKEVEDRQSAPYRGRTSILRDGITAGKAALRIHNV | |
| Sequence | TASDSGKYLCYFQDGDFYEKALVELKVADPFFRSAQPWIAALAGTLPILLLLLAGASY | |
| | FLWRQQKEITALSSEIESEQEMKEMGYAATEREISLRESLQEELKRKKIQYLTRGEES | |
| | SSDTNKSA | |
| | SEQ ID NO:165 | 1689 bp |
| NOV38c, | ATTTGCTTTCTCTTTTTCCTTTCTTCCGGATGAGAGGCTAAGCCATAATAGAAAGAAT | |
| CG59454-04 DNA | GGAGAATTATTCATTGACCGTCTTTATTCTGTGGGCTCTGATTCTCCAATGGGAATAC | |
| Sequence | CAAGCGATGGTTTTCCATACTGGAACCCAAAGGTAAAGACACTCAAGGACAGACATTT | |
| | TTGGCAGAGCATAGATGAAAATCGCAAGTTCCCTGGCTTTCCTTCTGCTCAACTTTCA | |
| | TGTCTCCCTCCTCTTGGTCCAGCTGCTCACTCCTTGCTCAGCTCAGTTTTCTGTGCTT | |
| | GGACCCTCTGGGCCCATCCTGGCCATGGTGGGTGAAGACGCTGATCTGCCCTGTCACC | |
| | CGTTCCCGACCATGAGTGCAGAGACCATGGAGCTGAAGTGGGTAAGTTCCAGCCTAAG | |
| | TATCGAGGGAGAACTTCGATTCTGCGGGATGCCATCACTGCACGGAAGGCTGCTCTCC | |
| | GAATACACAACGTCACAGCCTCTGACAGTGGAAAGTACTTGTGTTATTTCCAAGATGG | |
| | TGACTTCTATGAAAAAGCCCTGGTGGAGCTGAAGGTTGCAGCACTGGGTTCTAATCTT | |
| | CACGTCGAAGTGAAGGGTTATGAGGATGGAGGGATCCATCTGGAGTGCAGGTCCACCG | |
| | GCTGGTACCCCCAACCCCAAATACAGTGGAGCAACGCCAAGGGAGAGAACATCCCAGC | |
| | TGTGGAAGCACCTCTGGTTGCAGATGGAGTGGGCCTATATGAAGTAGCAGCATCTGTG | |
| | ATCATGAGAGGCGGCTCCGGGGAGGGTGTATCCTGCATCATCAGAAATTCCCTCCTCG | |
| | GCCTGGAAAAGACAGCCAGCATTTCCATCGCAGAGAGCCTCCAGGAGGAACTCAAGAG | |
| | GAAAAAATCCAGTACTTGACTCGTGGAGAGGAGTCTTCGTCCGATACCAATAAGTCAG | |
| | CCTGATGCTCTAATGGAAAAATGGCCCTCTTCAAGCCTGCAGATGTAATTCTGTATCC | |
| | AGACATGGCAAATGCCATCCTCCTTGTTTCTGAGGACCAGAGGAGTGTACAGCGTGCT | |
| | GAGGAGCCCCATGACCTACCAGACAACCCTGAGAGATTTGAATGGCGTTACTGTGTGC | |
| | TTGGCTGTGAAAGCTTCATGTCAGAGAGACACTACTCGGAGGTGGAAGTGGCGGACAG | |
| | AAAAGAGTGGCATATTGGGCTATGTAGTAAGAACGTGGAGAGGAAAAAAGTTTGGGTC | |
| | AAAATGACACCGGAGAACGGATACTGGACTATGGGCCTGACTGATGGGAATAAGTATC | |
| | GGGCTCTCACTGAGCCCAGAACCAACCTGAAACTTCCTGAGCCTCCTAGGAAAGTGGG | |
| | GGTCATCCTGGACTATGAGACTGGACATATCTCGTTCTACAATGCCACGGATGGATCT | |
| | CATATCTACACATTTCTGCACGCCTCTTCCTCTGAGCCTCTGTATCCTGTATTCAGAA | |
| | TTTTGACCTTGGAGCCCACTGCCCTGACCGTTTGCCCAATACCAAAAGTAGAGAGTTC | |
| | CCCCGATCCCGACCTAGTGCCTGATCATTCCCTGGAGATACCACTGACCCCAGGCTTA | |
| | GCTAATGAAAGTGGGCACCCTCAGGCTGAAGTAACATCTCTGCTTCTCCCTGCCCAGC | |
| | CTGGAGC | |
| | ORF Start: ATG at 189 | ORF Stop: TGA at 945 |
| | SEQ ID NO:166 | 252 aa MW at 27148.7 kD |

| | |
|---|---|
| NOV38c, | MKMASSLAFLLLNFHVSLLLVQLLTPCSAQFSVLGPSGPILANVGEDADLPCHPFPTM |
| CG59454-04 Protein | SAETMELKWVSSSLRQVVNVYADGKEVEDRQSAPYRGRTSILRDGITAGKAALRIHNV |
| Sequence | TASDSGKYLCYFQDGDFYEKALVELKVAALGSNLHVEVKGYEDGGIHLECRSTGWYPQ |
| | PQIQWSNAKGENIPAVEAPVVADGVGLYEVAASVIMRGGSGEGVSCIIRNSLLGLEKT |
| | ASISIAESLQEELKRKKSST |

Sequence comparison of the above protein sequences yields the following sequence relationships shown in Table 38B.

TABLE 38B
Comparison of NOV38a against NOV38b through NOV38c.

| | NOV38a Residues/ | Identities/Similarities for |
|---|---|---|
| Protein Sequence | Match Residues | the Matched Region |
| NOV38b | 33 . . . 151 | 118/119 (99%) |
| | 26 . . . 144 | 118/119 (99%) |
| NOV38c | 33 . . . 246 | 208/214 (97%) |
| | 26 . . . 239 | 210/214 (97%) |

Further analysis of the NOV38a protein yielded the following properties shown in Table 38C.

TABLE 38C
Protein Sequence Properties NOV38a

| | |
|---|---|
| PSort | 0.6850 probability located in endoplasmic reticulum |
| analysis: | (membrane); 0.6400 probability located in plasma membrane; |
| | 0.4600 probability located in Golgi body; |
| | 0.1000 probability located in endoplasmic reticulum (lumen) |
| SignalP | Cleavage site between residues 37 and 38 |
| analysis: | |

A search of the NOV38a protein against the Geneseq database, a proprietary database that contains sequences published in patents and patent publication, yielded several homologous proteins shown in Table 38D.

TABLE 38D
Geneseq Results for NOV38a

| | | NOV38a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAW99661 | Human secreted protein clone cc 130_1 | 8 . . . 341 | 323/334 (96%) | 0.0 |
| | protein - Homo sapiens, 334 aa. | 1 . . . 334 | 325/334 (96%) | |
| | [WO9907840-A1, 18-FEB-1999] | | | |
| AAW71592 | Human myelin oligodendrocyte | 8 . . . 334 | 315/327 (96%) | e−178 |
| | glycoprotein-like protein - Homo | 1 . . . 327 | 318/327 (96%) | |
| | sapiens, 331 aa. [WO9833912-A1, 06- | | | |
| | AUG-1998] | | | |
| AAW78917 | Bovine butyrophilin protein BTF4 - | 8 . . . 323 | 304/316 (96%) | e−172 |
| | Bos sp, 319 aa. [WO9814466-A1, 09- | 1 . . . 316 | 307/316 (96%) | |
| | APR-1998] | | | |
| AAW78916 | Bovine butyrophilin protein BTF3 - | 8 . . . 333 | 290/326 (88%) | e−165 |
| | Bos sp, 584 aa. [WO9814466-A1, 09- | 1 . . . 326 | 303/326 (91%) | |
| | APR-1998] | | | |
| AAW78918 | Bovine butyrophilin protein BTF5 - | 8 . . . 335 | 273/328 (83%) | e−156 |
| | Bos sp, 513 aa. [WO9814466-A1, 09- | 1 . . . 328 | 294/328 (89%) | |
| | APR-1998] | | | |

In a BLAST search of public sequence databases, the NOV38a protein was found to have homology to the proteins shown in the BLASTP data in Table 38E.

TABLE 38E
Public BLASTP Results for NOV38a

| | | NOV38a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NR44 | BUTYROPHILIN, SUBFAMILY 3, | 8 . . . 341 | 325/334 (97%) | 0.0 |
| | MEMBER A2 - Homo sapiens | 1 . . . 334 | 327/334 (97%) | |
| | (Human), 334 aa. | | | |
| Q9BU81 | SIMILAR TO BUTYROPHILIN, | 8 . . . 341 | 322/334 (96%) | 0.0 |
| | SUBFAMILY 3, MEMBER A2 | 1 . . . 334 | 325/334 (96%) | |
| | (DJ45P21.5) - Homo sapiens (Human), | | | |
| | 334 aa. | | | |
| P78410 | HYPOTHETICAL 39.4 KDA | 8 . . . 341 | 322/334 (96%) | 0.0 |
| | PROTEIN - Homo sapiens (Human), | 1 . . . 334 | 325/334 (96%) | |
| | 359 aa. | | | |
| O15338 | BUTYROPHILIN - Homo sapiens | 12 . . . 341 | 320/330 (96%) | e−180 |
| | (Human), 357 aa. | 3 . . . 332 | 322/330 (96%) | |
| O00477 | BUTYROPHILIN - Homo sapiens | 8 . . . 323 | 304/316 (96%) | e−171 |
| | (Human), 319 aa. | 1 . . . 316 | 307/316 (96%) | |

PFam analysis indicates that the NOV38a protein contains the domains shown in Table 38F.

TABLE 38F
Domain Analysis of NOV38a

| | | Identities/ | |
|---|---|---|---|
| | Nov38a | Similarities | Expect |
| Pfam Domain | Match Region | for the Matched Region | Value |
| ig: domain 1 of 1 | 52 . . . 135 | 14/85 (16%) | 0.00069 |
| | | 55/85 (65%) | |

### Example 39

The NOV39 clone was analyzed, and the nucleotide and polypeptide sequences are shown in Table 39A.

TABLE 39A
NOV39 Sequence Analysis

| | SEQ ID NO:167 | 9705 bp |
|---|---|---|
| NOV39a, | ATGGACCCTGCTCAGCGCCCGGCCCCTGGGGCTGCTATTGTCGAGGGCCTGCCCTCTC | |
| CG59307-01 DNA | CCTCAGACGCCTCCCTGGAGAGCCAAGCTGGTGTTCGAGGGTCCACCCTGCTGCCCAA | |
| Sequence | CAGCCCCGCGGCCACCAGAGGGCCGTCCCTGGCCCGGCTGTGTGCCCTGGTGGACCTG | |
| | TGTCTGGGCTGCTCCCGCTGCACCCAGCGGCTCAATGAAAGCACCTACGTCCTCCGTA | |
| | GGGTGGAGCATGACTGCTCCCGCGAGATCCTGCTGGCCCGCTTTAAGCAGGCCACCAA | |
| | GAGCAACGTCTGGCCCGTGGTGGGCTGCCGGCCCACCTTCCCAAGCCCCCTGTCCTAC | |
| | CAAGTCTGCCACTACTACAGCCCTGGGCTCGGCTGCCGGCGCCACCGAAACCGGTGCA | |
| | CCTTTGCCCGCAGTCGCCAGGAGGCCCTGGTCTGGACCTTCGAGCGTCAGCACAACCT | |
| | CCAGCGCCTATGGCTGAAGGCGGAGGTGCAGCGCAGCGGGGCCCAGGGAGGGGCAGGC | |
| | CGGGCGGCCGACGCCATCCTTACGGAGTTTGGCGGCCGCTTCGAGCTGCTTTGCTCCC | |
| | TCTGCTTCAGGCGCTGTCCCCCATGCATCTGTCGCGTGGACCCCCAGGGGCAGTGCCC | |
| | TGAGCACGGAGCCTGCCCCTCCCTCCTCCCCACGTGAGCCCCGAGGGCCGCCGCAAAG | |
| | CAACAGTTTGTGGTGGTGAGGCCGCGGCCCCGGGCCGGCCAGCCTCCTGCCTACTGCA | |
| | GGTTTGTGGGGCGTGGGCAGCCGTGCTGGCGTGGGGAGTCCCGCTGCCAGTTTGCACA | |
| | CAGCCCCGTGGAGATGGCTGTGTGGGAGGCCGAGCAGCTGGGTGGCCTCCAGCGCGGG | |
| | GACCTGCTCACACCCCCTGCCCCTGATGGCGACGGGCCCACGGCCCCCCTTGGCCAGC | |
| | CCCCTGGGGCCCAGCTGTACTGCCCGGCCTGCTTGGTCACCTGCCACTCTCAGGAGGC | |
| | CTTCGAGAACCACTGCGCATCCTCGGAGCACGCACAGATGGTGGCCTTCGACCAGCCC | |
| | CTGCCCTGGGACCACCGTTCCCCACCCCCGGGACTCTCCAAGTTCGAGCTCTGCCCAA | |
| | AGCCTGACCTCTGTGAGTATGGGGACGCCTGCACCAAGGCACACTCAGCACAGGAGCT | |
| | GCAGGAGTGGGTCCGGCGCACGCAGGCTGTGGAGCTGCGGGGGCAGGCGGCCTCGCAG | |
| | GACGGGCTGGTGCCCTACCAGGAGCGGCTGCTGGCCGAGTACCAGCGCAGCACCAGTG | |
| | AGGTCCTTGTGCTGGCAGAGACCCTTGATGGAGTGCGTGTCACCTGCAACCAGCCCCT | |
| | GATGTACCAGGCCCAGGAGAGGAAGACCCAGTACAGCTGGACGTTTGCCGTCCACTCT | |
| | GAGGAGCCCCTGCTACACGTGGCCCTGCTGAAGCAGGAGCCAGGAGCCGACTTCTCTC | |
| | TGGTGGCTCCCGGCCTCCCGCCAGGCCGGCTCTACGCACGGGGTGAGCGCTTCCGTGT | |
| | GCCCAGCTCCACTGCCCACTTCCAGGTGGGAGTGCGTGTGCAGGCTGCCTCGTTCGGC | |
| | ACCTTTGAGCAATGGGTGGTCTTCGACTTTGGCCGCCGGCCGGTGCTGCTACAAAAGC | |
| | TGGGGCTGCAGCTGGGCCAGGGGCGTCCCCCAGGACCCTGCAGGAATCTGGCGCTCGG | |
| | CCACCCTGAGGAGATGGAGCGCTGGCACACTGGCAACCGCCACGTGGTGCCTGGCGTC | |
| | GAGCGGACGGCCGAGCAGACGGCCCTGATGGCCAAGTACAAGGGCCCTGCCCTGGCCC | |
| | TGGAGTTCAACCGCAGCAGCGTGGCCTCGGGCCCCATCTCACCAACCAACTATCGGCA | |
| | GAGGATGCACCAGTTTCTCTATGAGGAGGAGGCGGCTCAGCAGCAGCTGGTGGCCAAG | |
| | CTGACCCTGCGGGCCCAGGTGTTCCTGAAGACGGCATTGCAGACGCCAGCGCTGAACA | |
| | TGCTCTTCGCGCCTCCGGGAGCACTGTACGCAGAGGTCCCCGTCCCCTCCTCCCTGAT | |
| | GCCAGACACAGACCAGGGCTTCCTGCTGGCCCGGGCGGTCAGCACAGCCCTGGTGGCC | |
| | CCTGTACCTGCACCCGACAATACGGTGTTCGAGGTGCGGCTGGAGAGGCGGGCCAGCT | |
| | CAGAGCAGGCGCTGTGGCTGCTGCTTCCGGCCCGCTGCTGCTTGGCCCTCGGGCTGCA | |
| | GCCTGAGGCCCGCCTGGTCCTGGAGGTGCAGTTCCAGATTGACCCGATGACCTTCCGC | |
| | CTCTGGCACCAGGCAGTGGACACACTGCCTGACGAGCAGCTGGTGGTGCCCGACTTGC | |
| | CCACCTGCGCCCTGCCCAGACCTTGGTCTGTCCCACCCTTGCGGCGTGGCAACCGCAA | |
| | GCAGGAGCTGGCCGTGGCGCTCATCGCGGGCTGGGGCCCTGGGGATGGGAGGCGTGTC | |
| | CCCCCGCTACTCATCTATGGCCCCTTTGGCACCGGCAAGACCTACACGCTGGCCATGG | |
| | CCTCCCTGGAGGTCATCCGGAGGCCTGAAACCAAGGTGCTCATCTGCACACACACCAA | |
| | CAGTCTACACCGGGCAGGCGGTCACCCCCTGGATGTGCTCCACTCCTCTGATTCTGCA | |
| | CTCCCTGTAGCCGACCAGCTTTGGGCTTTGGCTTTATCAGGCCCAGCTCAGGCCACCG | |
| | AGGGTGGCCGGGTCTGCTGTCAGGAGGGACACCAGTGTTTGCTGCTCACCTCTGACAG | |
| | CCAGACAAGGGCTGTGCTCAGGGGCAGCTCGGCTGGGCACACAGTAGGTGCTTTAGCG | |
| | GACAGCACTGAGGCCCCCAGCAAGAACCCCATGAGCTCCTCCCCCTCCCGCAGTGCCG | |
| | CCGACATCTACATCCGGGAGTATTTCCACAGCCACGTCAGCGGCGGCCACCCCGAGGC | |
| | CACTCCTCTCCGTGTGATGTACACGGACCCGCCGCTGAGCCAGACGGACCCAGTCACG | |
| | CTGCAGTACTGTTGCCTGACCCACGACCGCCAGGCTTTCCGCCCGCCCACACGGGCAG | |
| | AGCTGGCGCGGCACCGCGTGGTGGTCACCACCACCTCCCAGGCCCGTGAGCTCAGCCT | |
| | GCCGGTCGGCTTCTTCTCCCACATTCTCATCGATGAGGCGGCCCAGATGCTGGAGTGC | |
| | GAGGCCCTCACCCCGCTGGCCTATGCCTCGCACGGCACCCGCCTCGTGCTGGCGGGCG | |
| | ACCACATGCAGGTCACACCCCGGCTGTTCAGTGTGGCCAGGGCCCGGCCGGCCGAGCA | |
| | CACGCTGCTGCACCGCCTCTTCCTGTGCTACCAGCAGGAGACTCACGAGGTGGCGCGG | |
| | CAGAGCCGCCTGGTCTTCCACGAGAACTACCGCTGCACGGACGCCATTGTCAGCTTCA | |
| | TCTCGCGGCACTTCTACGTGGCCAAGGGCAACCCCATCCACGCCAGCGGCAAGGTTCC | |
| | GCCCCACCCCCGCCACTACCCGCTCATGTTCTGCCACGTGGCGGGCAGCCCAGACCGG | |
| | GACATGTCCATGGCGTCCTGGCTGAATCTGGCTGAGATTGCGCAGGTCGTCGAGAAGG | |
| | TGCAGGAGGCCTACAACACCTGGCCCAGCTGCTGGGGCGGCCGCGAGCAGAGGTGCAT | |
| | CTGTGTCGTTTCCCACGGTGCCCAGGTCAGTGCACTGAGGCAGGAGCTGAGGAGGCGG | |
| | GACCTAGGCCAGGTGTCTGTCGGCAGTTTTGAGATCCTGCCAGGGCGGCAGTTCCGGG | |
| | TCGTGGTGCTCAGCACTGTGCACACCTGCCAGAGCCTGCTCAGCCCTGGCGCACTCGC | |
| | CCCTGAGTTCTTCACCGACGCCCGCGTGCTCAACACCGTCCTGACCCGCGCCCAGTCC | |
| | CAGCTGGTGGTAGTGGGGGACGCCGTGGCCCTCTGCTCCTTCGGGGCCTGCGGCAAGC | |
| | TCTGGGAGAGCTTCATCCGTGAGTGCGTGGAGCGGCACAGTGTCTGCCCCGAGGGCCT | |
| | GTCCATGGAGCAGGTCGAGCAGGGTGTGGCGCAGAGACGGCGCTGGCCTCCCCGAGGC | |
| | ACACAGGCTGGGCCACCGGGCAACTGGGAGGCTGCCCCAGACCCAGTAGGGGACCTGG | |
| | CCGAGGAGCAGGCGGCTGTGGTGACGGCCATGGTGAAGGCAGAGCCGGGAGATGAGGC | |
| | TCTGAGCCCAGCATCCCGTGACATCACGGCAACCACAGCGCAGACGGAGGCTGCGGCA | |
| | GCACCAGCAGGAGACCCAGTGAAGGAAGACGTGGTGCCCGGGCCCTGTGCGGCAGGAG | |
| | CGGCTGCTGCAGCGGGCGTGGAGTCCACGGAGGCTGAGGATGCAGAGGCTGACTTCTG | |
| | GCCGTGGGACGGGGAGCTCAACGCTGACGACGCCATCCTACGGGAGCTTCTGGACGAG | |
| | AGCCAGAAGGTGATGGTGACCGTCGGGGAGGACGGGCTGCTGGACACTGTCGCCAGGC | |
| | CCGAGTCCCTGCAGCAGGCCCGGCTGTACGAGAACCTGCCCCCGGCTGCGCTACGGAA | |
| | GCTGCTGCACGCGGAGCCCGAGCGGTACCGCCACTGCTCTTTCGTGCCAGAGACCTTC | |
| | GACCGGGCGTCAGCCATCCCGCTGGACGATGCCTCCTCCGGCCCCATCCACGTCAGGG | |
| | GCCGCCTGGACTGTGGGATGGCCTTCGCCGGGGATGAGGTGCTGGTGCAGCTCCTTTC | |
| | GGGAGACAAGGCGCCCGAGGGGCGGCTTCGGGGCCGCGTGCTGGGCGTGCTGAAGAGG | |
| | AAGAGGCACGAGCTGGCGTTTGTGTGCCGCATGGACACGTGGGACCCGCGCATCATGC | |
| | TCCCCATCAATGGCTCCGTGACCAAGATCTTCGTGGCCGAGCTGAAGGACCCATCGCA | |
| | GGTCCCCATCTACAGCCTCCGGAAGGGCCGCCTGCAGCGTGTGGGGCTTGAGAGGCTC | |
| | ACCCCCGAGGCCCGGCACAGCCGGCTCTTCTGGGTCCAAATTGTCCTGTGGCGGCAAG | |
| | GCTTCTACTACCCGCTGGGCATCGTCCGGGAGGTGCTGCCTGAGGCCAGCACCTGGGA | |
| | GCAGGGCCTCCGCATCCTCGGCCTGCAGTACAGCTTGAGGGTGCCCCCGTCGGACCAG | |
| | GCCACCATCACCAAGGTGCTGCAGAAATACCACACGGAGCTTGGCCGCGTTGCCCGCC | |
| | GCCGAGAGGACTGCCGCGCCTTCTTGACCTTCACTGTGGACCCCCAGGGCGCCTGCAA | |
| | CCTCGATGATGCCCTCAGTGTCCGAGACCTGGGTCCCAGGTGCGAGGTGGCTGTGCAC | |
| | ATCACTGATGTGGCCAGCTTCGTGCCCAGGGACGGCGTGCTGGACGTGGAGGCGCGAA | |
| | GGCAGGGCGCTGCGTTCTATGCCCCCGGCAGGGAGCCAGTGCCCATGCTGCCGGCCAG | |
| | CCTCTGCCAGCACGTCCTCAGCCTCCTGCCTGGCCGGGACCGCCTGGCCATCTCCCTG | |
| | TTCCTCACCATGGAGAAGGCCAGTGGCCAGCTGAAGAGCCTGCGCTTTGCACCCTCCG | |
| | TGGTCCAGTCTGACCGCCAGCTGTCCTACGAGGAGGCGGAGGAGGTGATCAGGCAGCA | |
| | CCCGGGTGCCGGCCGTGAGCTGCCGGCCCGCCTGGACTCCGTGGACGCCTGCGTCGTG | |
| | GCCGCGTCCTACTTCTCTCCGCTGCTGCGCCGGCACCGCCTGCGGTCCGACTGCTTCT | |
| | ATGAGCAGCCGGACGAGGACGGCACCCTGGGCTTCCGCGCGGCCCACATCATGGTGAA | |
| | GGAGTACATGATTCAGTTTAATAGGCTCGTGGCTGAGTTCCTGGTGGGCAGCGAGTGC | |
| | ACGCGGACGGTCACGCCTCTGCGGTGGCAGCCAGCACCCCGCAGCCAGCAGCTCAAGG | |
| | CCCTGTGTGAGAAGCATGGGGACCGGGTGCCCCTGTCACTGCACCTCGGCCACCACCT | |
| | GCACGGCGGCGGGGGCAGTCCCCCCGACACGCGGCTGCACCTCCTGGCCTCCCTCTGG | |
| | AAGCAGGTCCAGTTTGCTGCCCGCACCCACCACTACCAACAGATGGTGGACTTGGTCA | |
| | CCACCGACGACATGCACCCATTCCTGGCTCCTGCAGGCCGCGACCTCCGCAAGGCCCT | |
| | GGAGCGCTCGGCGTTCGGCCGCTGCGCCCGGGGCCACCAGCAGCAGCGCGGCCACTAC | |
| | TCGCTGCAGGTGGACTGGTACACGTGGGCCACCTCGCCCATCCGCAGGTACCTGGACG | |
| | TGGTGTTGCAGCGGCAGATCCTGCTGGCGCTGGGCCATGCGGGCTCTGCCTACTCTGC | |
| | CAGGGACATCGATGGGCTCTGCCAGGCCTTCAGCCTCCAGCACGCACTTGCCCAGAGC | |
| | TATCAGCGGCGGGCGCGCAGCCTGCACCTGGCCGTGCAGCTCAAGGCCCAGCCTCTGG | |
| | ACAAGCTGGGCTTCGTGGTGGACCTGGAGGCGGGCTCCCGCTGCTTCCGGCTGCTCTT | |
| | CCCCAGCAACCGCGAGACGCTGCCTGACCCCTGCCCCGTCCCCTACGGCTCCCTGCAG | |
| | CTGGCCGAGCACCCCCACGCCCTGGCAGGCCGGCCGGGCCTGCGGCTCCTGTGGCGGC | |
| | GCCGTGTCTACTCAGCGCAGGGATCCAGCCCGCCCCTGCCACTGCCTGGCACTGTGCC | |
| | GGACCCACACACCCTGGCCGTGGAGACGGCCCTGTGGAAGCAGCTGCTGGAGCTGGTG | |
| | GAGCTGCAGCGCTGGCCGGAGGCGGCTGCTCTCATCCAGGAGAAGGGCGAGGCGTCCC | |
| | AGCGGCGGGAGCTGGTGCAGGTGCAGCGGAGCCACTGTGGCCATTTCCTGGAGGTGGC | |
| | CCGGGAGCTGGGCAGTGGGGACACCCTGCAGGTGCAGCTCGGCACCAGCCTCCAGCAC | |
| | GCTTCCTGGTACCGAGCCCTCAGCTCTGGACGGTGGCACCGGGCTTCAGCCTCTGCC | |
| | TGGAGCACGTGGAGCGGCCCGGAGACTGCTTCTCAGGCCGTGTGTACCGGGCCCCGAG | |
| | GGACCGGTACCGCGACCTGGATGAGTACGCCTGCGTGTGGGAACCATTCTGCGCCCTG | |
| | GAGTCGGCCACCGGCGCGGTTGCCGAGAATGACTCCGTCACACTTCAGCACCTGAGTG | |
| | TCTCCTGGGAGGCGTCACGGACGCCGCAGGGGCAGCTGCAGGGCGCCTTCCGCCTGGA | |
| | GGCCGCCTTCCTCGAGGAGAACTGTGCCGACATCAACTTCAGCTGCTGCTACCTCTGC | |
| | ATCCCCCTCGAGCCGCTGCCGGCTCCCACCGCCAGCCCACGCCCTGCGCCCAGCACCC | |
| | TCGGCCCTGGCCTGAATCTTGACCCCGGCACCTATACCTCGGTGGCCCACGGGCAGAC | |
| | CTCTTCGTCCACCACATGGGCATGGAGAAGGTTCCGGAAGAGGTGCTGAGGCCGGGCA | |
| | CCCTGTTCACCGTTGAGCTGCTGCCCAAGCAGCTTCCTGATCTCCGCAAGGAGGAAGC | |
| | CGTGCGTGGACTAGAGGAGGCGTCCCCGCTGGTCACCAGCATCGCACTGGGCCGGCCT | |
| | GTCCCGCAGCCCCTCTGCAGAGCCCCACCCTGCATGAGTGCTCAGGGAGGCTGCCCCC | |
| | TCTCAGTGATCCCCAGCAGGTTCCTGGAGCGGCAGACCTACAACATCCCCGGAGGCCG | |
| | CCACAAGCTGAACCCCAGCCACAACGTGGCGGTCAGGCACGCTCTGGAGAAGCCTTTC | |
| | ACGGTCATTCAGGGCCCACCAGGTACAGGGAAGACGATCGTGGGCCTCCACATCGTAT | |
| | TCTGGTTTCATAAATCAAACCAGGAGCAGGTGCAGCCCGGAGGCCCCCCCCGTGGGCA | |
| | GAAGCGGCTGGGGGGTCCCTCCATCTTGTACTGCGGCCCCTCCAACAAGTCGGTGGAT | |
| | GTCCTGGCAGGACTGCTCCTGAGAAGGATGGAGCTGAAGCCCCTCCGTGTGTACAGTG | |
| | AGCAGGCTGAGCCCAGCGACTTCCCAGTGCCGCGTGTGGGCAGCAGGAAGCTGCTCAG | |
| | GAAGAGCCCCCGGGAGGGGAGGCCGAACCAGAGCCTCAGGAGCATCACCCTGCACCAC | |
| | CGGATCCGCCAGGCCCCCAACCCTTACTCGTCGGAAATCAAGGCCTTTGACACCCGGC | |
| | TGCAGAGAGGGGAGCTCTTCTCCAGCGAGGACCTGGTCTGGTACAAGAAGGTCTTGTG | |
| | GGAGGCTCGGAAGTTCGAGCTGGACCGGCATGAGGTCATCCTCTGCACCTGCTCCTGT | |
| | GCAGCCTCTGCCAGCCTCAAAATCCTGGACGTGAGGCAGATCCTTCTTGACGAGGCAG | |
| | GCATGGCCACGCAACCTGAAACCCTCATCCCCCTGGTGCAGTTCCCACAGGCCGAGAA | |
| | GGTGGTTCTTCTCGGAGACCACAAGCAGCTGCGGCCTGTGGTCAAGAATGAGCGGCTG | |
| | CAAAACCTGGGTCTGGACCGGTCTCTGTTCGAGCGGTACCACGAGGACGCACATATGC | |
| | TGGACACTCAGTACCGCATGCATGAGGGCATCTGTGCCTTCCCCTCTGTCGCGTTCTA | |
| | CAAGAGCAACCTGAAGACGTGGCACGGCCTGAGGAGGCCGCCCAGTGTCCTGGGCCAC | |
| | GCTGGCAAGGAGAGCTGCCCTGTCATCTTTGGCCACGTGCAGCGCCACGAGCGGAGCC | |
| | TGCTGGTGTCCACGGACGAAGGGAATGAGAACTCCAAGGCCAACCTGGACGAGGTGGC | |
| | TGAGGTGGTCCGTATCACCAAGCAGCTGACCCTGGGGAGGACCGTAGAGCCCCAGGAC | |
| | ATCGCCGTCCTCACGCCCTACAACGCGCAGGCCTCTGAGATCAGCAAGGCCCTTCGGC | |
| | GAGAGGGCATCGCCCCGGTGGCCCTGTCCTCCATCACCAAGAGCCAGGGGAGCGAGTG | |
| | GCGCTATGTGCTGGTGAGCACCGTCCGCACCTGTCCCAAGAGCGACCTGGACCAGCGG | |
| | CCCACCAAGAGCTGGCTCAAGAAGTTTCTGGGCTTCGTTGTGGACCCCAACCAAGTGA | |
| | ATGTGGCTGTCACGCGGGCCCAGGAGGGGCTCTGCCTGATCGGTGAGGGCGGGGCTGG | |
| | GCTCTTCCAGGGTGGGAACACAGGAGACCACCTCCTTCTGCGCTGCTCCCCCCTCTGG | |
| | CGTAGCCTCCTGGACTTCTGCGAGGCTCAGCAGACCCTCGTGCCTGCCGGCCAGGCCC | |
| | GGTGTCTCCGGCTGCCTGTGTGGGCAGGGGAGGGCCGTGCCTGGTGTGGTGGGGACAA | |
| | GCCACAGCTCCAGAGCTGCTCCGGTGTCACCAGGCTGGCCAAGTCCAAAGTCCCTGAG | |
| | GCCACCAGCCTTGACTGTCCTGCTGGTCCCACTTTTAAAGCTGCTCCCCAGGACCCCC | |
| | TGGCCGCTGTGGACTGGGGTCCCTCCGCACCTGGCCCATTTGTGGCTGCGTCCACAGG | |
| | GGCTCCTGTGGCCTCCCAGAGCCAGCTCGGGGGTCAGATGGTCGCGGGGGCTATGGTC | |
| | ACTGTGGGAAAAGAGGTTCTGGGCATCTGTGGAGGGAGGGGTGGAGCATGGAGTCTCC | |
| | AGGACTGTGGCCCCGTTGGTGTGCTGGACGGGCCCTGCCTTGAAGACCATGTCTATTC | |
| | TTGGACCGTCATGAAATAA | |
| | ORF Start: ATG at 1 | ORF Stop: TAA at 9703 |
| | SEQ ID NO:168 | 3234 aa MW at 355870.4 kD |

| | |
|---|---|
| NOV39a, | MDPAQRPAPGAAIVEGLPSPRSSNVIGLRRGSLVVDGPPSRPPEVADTATAKASTWAS |
| CG59307-01 Protein | GLRTVANSSSGLRCPVSSGASDASLESQAGVRGSTLLPNSPAATRGPSLARLCALVDL |
| Sequence | CLGCSRCTQRLNESTYVLRRVEHDCSREILLARFKQATKSKVWRVVGCRPTFPRPLCY |
| | QVCHYYSPGLGCRRHRNRCTFARSREEALVWTFERQHNLQRLWLKAEVQGSGAQGGAG |
| | RAADAILTEFGGRFELLCSLCFRRCPPCICRVDPQGQCPEHGACPSLLAHVSAEGRRK |
| | QQFVVVRPRPRACQPPAYCRFVGRGQPCWRGESRCQFAHSAVEMAVWEAEQLGGLQRG |
| | DLLTPPAPDGDGRTAPLGQPPGAQLYCPACLVTCHSQEAFENHCASSEHAQMVAFDQA |
| | LPWEHRSPPPGLSKFELCPKPDLCEYGDACTKAHSAQELQEWVRRTQAVELRGQAAWQ |
| | DGLVPYQERLLAEYQRSSSEVLVLAETLDGVRVTCNQPLMYQAQERKTQYSWTFAVHS |
| | EEPLLHVALLKQEPGADFSLVAPGLPPGRLYARGERFRVPSSTADFQVGVRVQAASFG |
| | TFEQWVVFDFGRRPVLLQKLGLQLGQGRRPGPCRNLALGHPEEMERWHTCNRHVVPGV |
| | ERTAEQTALMAKYKGPALALEFNRSSVASGPISPTNYRQRMHQFLYEEEAAQQQLVAK |
| | LTLRGQVFLKTALQTPALNMLFAPPGALYAEVPVPSSLMPDTDQGFLLGRAVSTALVA |
| | PVPAPDNTVFEVRLERRASSEQALWLLLPARCCLALGLQPEARLVLEVQFQIDPMTFR |
| | LWHQAVDTLPEEQLVVPDLPTCALPRPWSVPPLRRGNRKQELAVALIAGWGPGDGRRV |
| | PPLLIYGPFGTGKTYTLAMASLEVIRRPETKVLICTHTNSLHRACGHPLDVLQSSDSA |
| | LPVADQLWALALSGPAQGTEGGRVCCQEGHQCLLLTSDSQTRAVLRGSSAGHTVGALA |
| | DSTEAPSKKPMSSSPSRSAADIYIREYFHSHVSGGHPEATPLRVMYTDRPLSQTDPVT |
| | LQYCCLTDDRQAFRPPTRAELARHRVVVTTTSQARELRVPVGFFSHILIDEAAQMLEC |
| | EALTPLAYASHGTRLVLAGDHMQVTPRLFSVARARAAEHTLLHRLFLCYQQETHEVAR |
| | QSRLVFHENYRCTDAIVSFISRHFYVAKGNPIHARGKVPPHPRHYPLMFCHVAGSPDR |
| | DMSMASWLNLAEIAQVVEKVQEAYNTWPSCWGGREQRCICVVSHGAQVSALRQELRRR |
| | DLGQVSVGSFEILPGRQFRVVVLSTVHTCQSLLSPGALAPEFFTDARVLNTVLTRAQS |
| | QLVVVGDAVALCSFGACGKLWESFIRECVERHSVCPEGLSMEQVEQGVAQRRRWPPRG |
| | TQAGAAGNWEAAPEPVGDLAEEQAAVVTAMVKAEPGDEALSPASRDITATTAQTEAAA |
| | APAGDAVKEDVVPGACAAGAAAAAGVESTEAEDAEADFWPWDGELNADDAILRELLDE |
| | SQKVMVTVGEDGLLDTVARPESLQQARLYENLPPAALRKLLHAEPERYRHCSFVPETF |
| | ERASAIPLDDASSGPIQVRGRLDCGMAFAGDEVLVQLLSGDKAPEGRLRGRVLGVLKR |
| | KRHELAFVCRMDTWDPRIMVPINGSVTKIFVAELKDPSQVPIYSLRKGRLQRVGLERL |
| | TAEARHSRLFWVQIVLWRQGFYYPLCIVREVLPEASTWEQGLRILGLEYSLRVPPSDQ |
| | ATITKVLQKYHTELGRVAGRREDCRAFLTFTVDPQGACNLDDALSVRDLGPRCEVAVH |
| | ITDVASFVPRDGVLDVEARRQGAAFYAPGREPVPMLPASLCQDVLSLLPGRDRLAISL |
| | FLTMEKASGQLKSLRFAPSVVQSDRQLSYEEAEEVIRQHPGAGRELPARLDSVDACVV |
| | AACYFSRLLRRHRLRSDCFYEQPDEDGTLGFRAAHIMVKEYMIQFNRLVAEFLVGSEC |
| | TRTVTPLRWQPAPRSQQLKALCEKHGDRVPLSLHLGHHLHGGGGSPPDTRLHLLASLW |
| | KQVQFAARTQDYEQMVDLVTTDDMHPFLAPAGRDLRKALERSAFGRCARGHQQQGGHY |
| | SLQVDWYTWATSPIRRYLDVVLQRQILLALGHGGSAYSARDIDGLCQAFSLQHALAQS |
| | YQRRARSLHLAVQLKAQPLDKLGFVVDVEAGSRCFRLLFPSNRETLPDPCPVPYGSLQ |
| | LAEHPHALAGRPGLRLLWRRRVYSAQGSSPPLPLPGTVPDPHTLAVETALWKQLLELV |
| | ELQRWPEAAALIQEKGEASQRRELVQVQRSHCGHFLEVARELGSGDTLQVQLGTSLQH |
| | GFLVPSPQLWTVAPGFSLCLEHVERPGDCFSGRVYRAPRDRYRDVDEYACVWEPFCAL |
| | ESATGAVAENDSVTLQHLSVSWEASRTPQGQLQGAFRLEAAFLEENCADINFSCCYLC |
| | IRLEGLPAPTASPRPGPSSLGPGLNVDPGTYTWVAHGQTQDWDQERRADRQEAPRRVH |
| | LFVHHMCMEKVPEEVLRPGTLFTVELLPKQLPDLRKEEAVRGLEEASPLVTSIALGRP |
| | VPQPLCRAPPCMSAQGGCPLSVIPSRFLERQTYNIPGGRHKLNPSQNVAVREALEKPF |
| | TVIQGPPGTGKTIVGLHIVFWFHKSNQEQVQPGGPPRGEKRLGGPCILYCGPSNKSVD |
| | VLAGLLLRRMELKPLRVYSEQAEASEFPVPRVGSRKLLRKSPREGRPNQSLRSITLHH |
| | RIRQAPNPYSSEIKAFDTRLQRGELFSREDLVWYKKVLWEARKFELDRHEVILCTCSC |
| | AASASLKILDVRQILVDEAGMATEPETLIPLVQFPQAEKVVLLGDHKQLRPVVKNERL |
| | QNLGLDRSLFERYHEDAHMLDTQYRMHEGICAFPSVAFYKSKLKTWQGLRRPPSVLGH |
| | AGKESCPVIFGHVQGHERSLLVSTDEGNENSKANLEEVAEVVRITKQLTLGRTVEPQD |
| | IAVLTPYNAQASEISKALRREGIAGVAVSSITKSQGSEWRYVLVSTVRTCAKSDLDQR |
| | PTKSWLKKFLGFVVDPNQVNVAVTRAQEGLCLIGEGGAGLFQGGNTGDHLLLRCCPLW |
| | RSLLDFCEAQQTLVPAGQGRCLRLPVWGGEGRAWCGGDKPQLQSCSGVTRLAKSKVPE |
| | ATSLDCPAGPTFKAAPQDPLAAVDWGPSAPGPFVAASTGAPVASQSQLGGQMVAGAMV |
| | TVGKEVLGICGGRGGAWSLQDCGPVGVLDGPCLEDHVYSWTVMK |

Further analysis of the NOV39a protein yielded the following properties shown in Table 39B.

TABLE 39B
Protein Sequence Properties NOV39a

| | |
|---|---|
| PSort | 0.7000 probability located in plasma membrane; |
| analysis: | 0.3500 probability located in nucleus; |
| | 0.3000 probability located in microbody (peroxisome); |
| | 0.2000 probability located in endoplasmic |
| | reticulum (membrane) |
| SignalP | No Known Signal Sequence Indicated |
| analysis: | |

A search of the NOV39a protein against the Geneseq database, a proprietary database that contains sequences published in patents and patent publication, yielded several homologous proteins shown in Table 39C.

TABLE 39C
Geneseq Results for NOV39a

| | | NOV39a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAB94712 | Human protein sequence SEQ ID | 334 . . . 523 | 190/190 (100%) | e−112 |
| | NO: 15712 - Homo sapiens, 293 aa. | 1 . . . 190 | 190/190 (100%) | |
| | [EP 1074617-A2, 07-FEB-2001] | | | |
| AAU23357 | Novel human enzyme polypeptide | 1759 . . . 1901 | 141/143 (98%) | 5e−75 |
| | #443 - Homo sapiens, 143 aa. | 1 . . . 143 | 141/143 (98%) | |
| | [WO200155301-A2, 02-AUG-2001] | | | |
| ABB25190 | Protein #7189 encoded by probe for | 817 . . . 909 | 93/93 (100%) | 4e−49 |
| | measuring heart cell gene expression - | 1 . . . 93 | 93/93 (100%) | |
| | Homo sapiens, 93 aa. [WO200157274- | | | |
| | A2, 09-AUG-2001] | | | |
| AAM74813 | Human bone marrow expressed probe | 817 . . . 909 | 93/93 (100%) | 4e−49 |
| | encoded protein SEQ ID NO: 35119 - | 1 . . . 93 | 93/93 (100%) | |
| | Homo sapiens, 93 aa. [WO200157276- | | | |
| | A2, 09-AUG-2001] | | | |
| AAM62011 | Human brain expressed single exon | 817 . . . 909 | 93/93 (100%) | 4e−49 |
| | 34116 - Homo sapiens, 93 aa. | 1 . . . 93 | 93/93 (100%) | |
| | [WO200157275-A2, 09-AUG-2001] | | | |

In a BLAST search of public sequence databases, the NOV39a protein was found to have homology to the proteins shown in the BLASTP data in Table 39D.

TABLE 39D
Public BLASTP Results for NOV39a

| | | NOV39a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9C094 | KIAA1769 PROTEIN - Homo sapiens | 963 . . . 3099 | 2102/2137 (98%) | 0.0 |
| | (Human), 2114 aa (fragment). | 1 . . . 2110 | 2104/2137 (98%) | |
| Q9BYK8 | DJ697K14.6 (NOVEL PROTEIN, | 992 . . . 3099 | 1985/2108 (94%) | 0.0 |
| | KIAA1769) - Homo sapiens (Human), | 565 . . . 2563 | 1988/2108 (94%) | |
| | 2567 aa. | | | |
| Q9BGV5 | HYPOTHETICAL 60.5 KDA | 2244 . . . 2754 | 460/511 (90%) | 0.0 |
| | PROTEIN - Macaca fascicularis | 3 . . . 499 | 475/511 (92%) | |
| | (Crab eating macaque) (Cynomolgus | | | |
| | monkey), 545 aa. | | | |
| Q922U3 | UNKNOWN (PROTEIN FOR | 2453 . . . 3090 | 463/694 (66%) | 0.0 |
| | IMAGE: 3589116) - Mus musculus | 1 . . . 681 | 529/694 (75%) | |
| | (Mouse), 717 aa (fragment). | | | |
| Q96ND3 | CDNA FLJ31049 FIS, CLONE | 2678 . . . 3099 | 405/422 (95%) | 0.0 |
| | HSYRA2000510, WEAKLY | 1 . . . 409 | 405/422 (95%) | |
| | SIMILAR TO NAM7 PROTEIN - | | | |
| | Homo sapiens (Human), 413 aa. | | | |

PFam analysis indicates that the NOV39a protein contains the domains shown in Table 39E.

TABLE 39E
Domain Analysis of NOV39a

| | | Identities/ | |
|---|---|---|---|
| | | Similarities | |
| | NOV39a Match | for the Matched | Expect |
| Pfam Domain | Region | Region | Value |
| zf-CCCH: domain | 304 . . . 331 | 11/28 (39%) | 1.1 |
| 1 of 2 | | 19/28 (68%) | |
| zf-CCCH: domain | 419 . . . 442 | 11/27 (41%) | 2.3 |
| 2 of 2 | | 16/27 (59%) | |
| Runt: domain | 1734 . . . 1756 | 6/23 (26%) | 0.7 |
| 1 of 1 | | 18/23 (78%) | |
| RNB: domain | 1682 . . . 2036 | 98/414 (24%) | 2.1e−29 |
| 1 of 1 | | 240/414 (58%) | |
| UvrD-helicase: domain | 2594 . . . 2622 | 14/29 (48%) | 2.4 |
| 1 of 2 | | 23/29 (79%) | |
| UPF0066: domain | 2591 . . . 2692 | 25/138 (18%) | 5.3 |
| 1 of 1 | | 62/138 (45%) | |
| UvrD-helicase: domain | 2951 . . . 2981 | 15/32 (47%) | 1.7 |
| 2 of 2 | | 25/32 (78%) | |

### Example 40

The NOV40 clone was analyzed, and the nucleotide and polypeptide sequences are shown in Table 40A.

TABLE 40A
NOV40 Sequence Analysis

| | SEQ ID NO:169 | 11603 bp |
|---|---|---|
| NOV40a, | CCCTCCATTGCTATGGATACCGAATCCACTTATTCTGGATATTCTTACTATTCAAGTC | |
| CG59713-01 DNA | ATTCGAAAAAATCTCACAGACAAAGGGAAAGAACTAGAGAGAGACACAAGTCACCCCG | |
| Sequence | GAATAAAGACGGCAGAGGGTCAGAAAAGTCTGTCACCATTCAACCTCCCACTGGAGAG | |
| | CCCCTGTTGGGAAATGATTCTACTCGGACAGAGGAACAGGATGACAACTGGGGAGAGA | |
| | CCACCACGGCCATCACAGGCACCTCGGAGCACAGCATATCCCAAGAGGACATTGCCAG | |
| | GATCAGCAAGGACATGGAGGACAGCGTGGGGCTGGATTGCAAACGCTACCTGGGCCTC | |
| | ACCGTCGCCTCTTTTCTTGGACTTCTAGTTTTCCTCACCCCTATTGCCTTCATCCTTT | |
| | TACCTCCCATCCTGTGGAGGGATGAGCTGGAGCCTTGTGGCACAATTTGTGAGGGGCT | |
| | CTTTATCTCCATGGCATTCAAACTCCTCATTCTGCTCATAGCGACCTGCGCACTTTTT | |
| | TTCCGCAAGCGGAGAGCTGACATGCCACGGGTGTTTGTGTTTCGTGCCCTTTTGTTGG | |
| | TCCTCATCTTTCTCTTTGTGGTTTCCTATTGGCTTTTTTACGGGGTCCGCATTTTGGA | |
| | CTCTCGGGACCGGAATTACCAGGGCATTGTGCAATATGCAGTCTCCCTTGTGGATGCC | |
| | CTCCTCTTCATCCATTACCTGGCCATCGTCCTGCTCGAGCTCAGGCAGCTGCAGCCCA | |
| | TGTTCACGCTGCAGGTGGTCCGCTCCACCGATGGCGAGTCCCGCTTCTACAGCCTGGG | |
| | ACACCTCAGTATCCAGCGAGCAGCATTGGTGGTCCTAGAAAATTACTACAAAGATTTC | |
| | ACCATCTATAACCCAAACCTCCTAACAGCCTCCAAATTCCGAGCAGCCAAGCATATGG | |
| | CCGGGCTGAAAGTCTACAATGTATTCCCCAAAGGCCCCAGTAACAATGCCACTGGCCA | |
| | GTCCCGGGCCATGATTGCTGCAGCTGCTCGGCGCAGGGACTCAAGCCACAACGAGTTG | |
| | TATTATCAAGAGGCCGAACATGAACGGCGAGTAAAGAAGCGGAAAGCAAGGCTGGTGG | |
| | TTGCAGTGGAAGAGGCCTTCATCCACATTCAGCGTCTCCAGGCTGAGCAGCAGCAGAA | |
| | AGCCCCAGGGGAGGTGATGGACCCTAGGGAGGCCGCCCACGCCATTTTCCCCTCCATG | |
| | GCCAGGGCTCTCCAGAAGTACCTGCGCATCACCCGGCAGCAGAACTACCACAGCATGG | |
| | AGAGCATCCTGCAGCACCTGGCCTTCTGCATCACCAACGCCATGACCCCCAAGGCCTT | |
| | CCTAGAACGGTACCTCAGTGCGGGCCCCACCCTGCAATATGACAAGGACCGCTGGCTC | |
| | TCTACACAGTGGAGGCTTGTCAGTGATGAGGCTGTGACTAATGGATTACGGGATGGAA | |
| | TTGTGTTCGTCCTTAAGTGCTTGGACTTCAGCCTCGTAGTCAATGTGAAGAAAATTCC | |
| | ATTCATCATACTCTCTGAAGAGTTCATAGACCCCAAATCTCACAAATTTGTCCTTCGC | |
| | TTACAGTCTGAGACATCCGTTTAAAAGTTCTATATTT | |
| | ORF Start: ATG at 13 | ORF Stop: TAA at 1588 |
| | SEQ ID NO:170 | 525aa MW at 60231.6 kD |
| NOV40a, | MDTESTYSGYSYYSSHSKKSHRQRERTRERHKSPRNKDGRGSEKSVTIQPPTGEPLLG | |
| CG59713-01 Protein | NDSTRTEEQDDNWGETTTAITGTSEHSISQEDIARISKDMEDSVGLDCKRYLGLTVAS | |
| Sequence | FLGLLVFLTPIAFILLPPILWRDELEPCGTICEGLFISMAFKLLILLIGTWALFFRKR | |
| | RADMPRVFVFRALLLVLIFLFVVSYWLFYGVRILDSRDRNYQGIVQYAVSLVDALLFI | |
| | HYLAIVLLELRQLQPMFTLQVVRSTDGESRFYSLGHLSIQRAALVVLENYYKDFTIYN | |
| | PNLLTASKFRAAKHMAGLKVYNVFPKGPSNNATGQSRAMIAAAARRRDSSHNELYYEE | |
| | AEHERRVKKRKARLVVAVEEAFIHIQRLQAEEQQKAPGEVMDPREAAQAIFPSMARAL | |
| | QKYLRITRQQNYHSMESILQHLAFCITNGMTPKAFLERYLSAGPTLQYDKDRWLSTQW | |
| | RLVSDEAVTNGLRDGIVFVLKCLDFSLVVNVKKIPFIILSEEFIDPKSHKFVLRLQSE | |
| | TSV | |
| | SEQ ID NO:171 | 342 bp |
| NOV40b, | GGATCCATGGATACCGAATCCACTTATTCTGGATATTCTTACTATTCAAGTCATTCGA | |
| 170645777 DNA | AAAAATCTCACAGACAAGGGGAAAGAACTAGAGAGAGACACAAGTCACCCCGGAATAA | |
| Sequence | AGACGGCAGAGGGTCAGAAAAGTCTGTCACCATTCAACCTCCCACTGGAGGGCCCCTG | |
| | TTGGGAAATGATTCTACTCGGACAGAGGAAGTTCAGGATGACAACTGGGGAGAGACCA | |
| | CCACGGCCATCACAGGCACCTCGGAGCACAGCATATCCCAAGAGGACATTGCCAGGAT | |
| | CAGCAAGGACATGGAGGACAGCGTGGGGCTGGATTGCAAACGCTACCTCGAG | |
| | ORF Start: GGA at 1 | ORF Stop: b at 343 |
| | SEQ ID NO:172 | 114aa MW at 12715.5 kD |

| | |
|---|---|
| NOV40b, | GSMDTESTYSGYSYYSSHSKKSHRQGERTRERHKSPRNKDGRGSEKSVTIQPPTGGPL |
| 170645777 Protein | LGNDSTRTEEVQDDNWGETTTAITGTSEHSISQEDIARISKDMEDSVGLDCKRYLE |
| Sequence | |

Sequence comparison of the above protein sequences yields the following sequence relationships shown in Table 40B.

TABLE 40B
Comparison of NOV40a against NOV40b and NOV40c.

| | NOV40a Residues/ | Identities/Similarities for the |
|---|---|---|
| Protein Sequence | Match Residues | Matched Region |
| NOV40b | 1 . . . 110 | 91/111 (81%) |
| | 3 . . . 113 | 91/111 (81%) |

Further analysis of the NOV40a protein yielded the following properties shown in Table 40C.

TABLE 40C
Protein Sequence Properties NOV40a

| | |
|---|---|
| PSort | 0.6000 probability located in plasma membrane; |
| analysis: | 0.4000 probability located in Golgi body; |
| | 0.3000 probability located in endoplasmic reticulum |
| | (membrane); 0.3000 probability located in |
| | microbody (peroxisome) |
| SignalP | No Known Signal Sequence Indicated |
| analysis: | |

A search of the NOV40a protein against the Geneseq database, a proprietary database that contains sequences published in patents and patent publication, yielded several homologous proteins shown in Table 40D.

TABLE 40D
Geneseq Results for NOV40a

| | | NOV40a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAY73493 | Human secreted protein clone yk224_1 | 98 . . . 525 | 425/428 (99%) | 0.0 |
| | protein sequence SEQ ID NO: 208 - | 1 . . . 426 | 425/428 (99%) | |
| | Homo sapiens, 426 aa. [WO9958642- | | | |
| | A2, 18-NOV-1999] | | | |
| AAG81268 | Human AFP protein sequence SEQ ID | 178 . . . 525 | 343/348 (98%) | 0.0 |
| | NO: 54 - Homo sapiens, 346 aa. | 1 . . . 346 | 343/348 (98%) | |
| | [WO200129221-A2, 26-APR-2001] | | | |
| AAY86275 | Human secreted protein HSRGW16, | 269 . . . 525 | 254/257 (98%) | e−141 |
| | SEQ ID NO: 190 - Homo sapiens, 295 | 41 . . . 295 | 254/257 (98%) | |
| | aa. [WO9966041-A1, 23-DEC-1999] | | | |
| AAB45189 | Human secreted protein sequence | 359 . . . 525 | 166/167 (99%) | 2e−90 |
| | encoded by gene 18 SEQ ID NO: 130 - | 1 . . . 167 | 166/167 (99%) | |
| | Homo sapiens, 167 aa. [WO200058467- | | | |
| | A1, 05-OCT-2000] | | | |
| AAY86492 | Human gene 61-encoded protein | 258 . . . 426 | 160/169 (94%) | 3e−83 |
| | fragment, SEQ ID NO: 407 - Homo | 42 . . . 208 | 161/169 (94%) | |
| | sapiens, 236 aa. [WO9966041-A1, 23- | | | |
| | DEC-1999] | | | |

In a BLAST search of public sequence databases, the NOV40a protein was found to have homology to the proteins shown in the BLASTP data in Table 40E.

TABLE 40E
Public BLASTP Results for NOV40a

| | | NOV40a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q923Z8 | LTAP - Mus musculus (Mouse), | 1 . . . 525 | 382/525 (72%) | 0.0 |
| | 521 aa. | 1 . . . 521 | 455/525 (85%) | |
| Q9ULK5 | KIAA1215 PROTEIN - Homo | 1 . . . 525 | 384/525 (73%) | 0.0 |
| | (fragment). | 55 . . . 575 | 453/525 (86%) | |
| AAK63188 | LPP1 - Mus musculus (Mouse), | 1 . . . 525 | 380/525 (72%) | 0.0 |
| | 521 aa. | 1 . . . 521 | 454/525 (86%) | |
| Q90X64 | STRABISMUS - Xenopus laevis | 1 . . . 525 | 383/525 (72%) | 0.0 |
| | (African clawed frog), 521 aa. | 1 . . . 521 | 448/525 (84%) | |
| Q91ZD4 | LPPI - Mus musculus (Mouse), | 1 . . . 525 | 380/526 (72%) | 0.0 |
| | 522 aa. | 1 . . . 522 | 454/526 (86%) | |

PFam analysis indicates that the NOV40a protein contains the domains shown in the Table 40F.

TABLE 40F
Domain Analysis of NOV40a

| | | | |
|---|---|---|---|
| | | Identities/ | |
| | NOV40a | Similarities for the | |
| Pfam Domain | Match Region | Matched Region | Expect Value |

No Significant Matches Found

### Example 41

The NOV41 clone was analyzed, and the nucleotide and polypeptide sequences are shown in Table 41A.

TABLE 41A
NOV41 Sequence Analysis

| | SEQ ID NO:173 | 1066 bp |
|---|---|---|
| NOV41a, | GACAAAACATGGTTCAAGCATCTGGGCACAGGCGGTCCACCCGTGGCTCCAAAATGGT | |
| CG59570-01 DNA | CTCCTGGTCCGTGATAGCAAAGATCCAGGAAATATCGTGCGAGGAAGATGAGAGGAAG | |
| Sequence | ATGGTGCGAGAGTTCTTGGCCGAGTTCATGAGCACATATGTCATGATGGTATTCGGCC | |
| | TTGGTTCTGTGGCCCATATGGTTCTAAATAAAACATATGGGAGCTACCTTGGTGTCAA | |
| | CTTGGGTTTTGGCTTCGGGGTCACCATGGGAGTCCACGTGGCAGGCCGCATCTCTGGA | |
| | GCCCACATGAATGCAGCTGTGACCTTCACTAACTGTGCGCTGGGCCGCGTGCCCTGGA | |
| | GGAAGTTTCCAGTCCATGTGCTGGGGCAGTTCCTGGGCTCCTTCCTGGCAGCTGCCAC | |
| | CATCTACAGTCTCTTCTACAGCGCCATTCTCCACTTTTCGGGTGGAGAGCTGATGGTG | |
| | ACCGGTCCCTTTGCTACAGCTGGCATTTTTGCCACCTACCTTCCTGATCACATGACAT | |
| | TGTGGCGGGGCTTCCTGAATGAGGAGTGGCTGACCAGGATGCTCCAGCTGTGTCTCTT | |
| | CACCATCACGGACCAGGAGAACAACCCAGCACTGCCAGGAACACACGCGCTGGTGATA | |
| | AGCATCCTCGTGGTCATCATCAGGGTGTCCCATGGCATAAACACAGGATATGCCATCA | |
| | ATCCATCCCGGGACCCGCCCCCCAGCATCTTCACCTTCATTGCTGGCTGGGGCAAACA | |
| | GGTCTTCAGCGATGGGGAGAACTGGTGGTGGGTGCCAGTGGTGGCACCACTTCTGGGT | |
| | GCCTCTCTAGGTGGCATCATCTACCTGGTCTTCATTGGCTCCACCATCCCACGGGAGC | |
| | CCCTGAAATTGGAGGACTCTGTGGCGTATGAAGACCACGGGATAACCGTATTGCCCAA | |
| | GATGGGATCTCATGAACCCATGATCTCTCCCCTCACCCTCATCTCCGTGAGCCTTGCC | |
| | AACAGATCTTCAGTCCACTCTGCCCCACCCTTACATGAATCCATGGCCCTAGAGCACT | |
| | TCTAAGCAGAGATTATTTGTGA | |
| | ORF Start: ATG at 9 | ORF Stop: TAA at 1047 |
| | SEQ ID NO:174 | 346 aa MW at38029.9 kD |

| | |
|---|---|
| NOV41a, | MVQASGHRRSTRGSKMVSWSVIAKIQEIWCEEDERKMVREFLAEFMSTYVMMVFGLGS |
| CG59570-01 Protein | VAHMVLNKTYGSYLGVNLGFGFGVTMGVHVAGRISGAHMNAAVTFTNCALGRVPWRKF |
| Sequence | PVHVLGQFLGSFLAAATIYSLFYSAILHFSGGELMVTGPFATAGIFATYLPDHMTLWR |
| | GFLNEEWLTRMLQLCLFTITDQENNPALPGTHALVISILVVIIRVSHGINTGYAINPS |
| | RDPPPSIFTFIAGWGKQVFSDGENWWWVPVVAPLLGASLGGIIYLVFIGSTIPREPLK |
| | LEDSVAYEDHGITVLPKNGSHEPMISPLTLISVSLANRSSVHSAPPLHESMALEHF |

Further analysis of the NOV41a protein yielded the following properties shown in Table 41B.

TABLE 41B
Protein Sequence Properties NOV41a

| | |
|---|---|
| PSort | 0.8586 probability located in mitochondrial inner membrane; |
| analysis: | 0.6000 probability located in plasma membrane; |
| | 0.4000 probability located in Golgi body; |
| | 0.3568 probability located in mitochondrial |
| | intermembrane space |
| SignalP | No Known Signal Sequence Indicated |
| analysis: | |

A search of the NOV41 a protein against the Geneseq database, a proprietary database that contains sequences published in patents and patent publication, yielded several homologous proteins shown in Table 41C.

TABLE 41C
Geneseq Results for NOV41a

| | | NOV41a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAW87644 | A protein with water channel activity - | 1 . . . 346 | 316/346 (91%) | 0.0 |
| | Homo sapiens, 342 aa. [WO9843997- | 1 . . . 342 | 323/346 (93%) | |
| | A1, 08-OCT-1998] | | | |
| AAY70455 | Human membrane channel protein-5 | 5 . . . 346 | 312/343 (90%) | e−179 |
| | (MECHP-5) - Homo sapiens, 341 aa. | 3 . . . 341 | 319/343 (92%) | |
| | [WO200012711-A2, 09-MAR-2000] | | | |
| AAY70468 | Human membrane channel protein-18 | 37 . . . 296 | 104/261 (39%) | 7e−56 |
| | (MECHP-18) - Homo sapiens, 301 aa. | 20 . . . 280 | 162/261 (61%) | |
| | [WO200012711-A2, 09-MAR-2000] | | | |
| AAB65991 | Human secreted protein BLAST search | 133 . . . 280 | 62/148 (41%) | 9e−33 |
| | protein SEQ ID NO: 131 - Homo | 1 . . . 148 | 99/148 (66%) | |
| | sapiens, 177 aa. [WO200077023-A1, | | | |
| | 21-DEC-2000] | | | |
| AAB65992 | Human secreted protein BLAST search | 133 . . . 280 | 62/148 (41%) | 4e−31 |
| | protein SEQ ID NO: 132 - Homo | 1 . . . 148 | 96/148 (63%) | |
| | 21-DEC-2000] | | | |

In a BLAST search of public sequence databases, the NOV41a protein was found to have homology to the proteins shown in the BLASTP data in Table 41D.

TABLE 41D
Public BLASTP Results for NOV41a

| | | NOV41a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD13298 | BA251O17.3 (SIMILAR TO | 1 . . . 346 | 345/346 (99%) | 0.0 |
| | AQUAPORIN 7) - Homo sapiens | 1 . . . 346 | 346/346 (99%) | |
| | (Human), 346 aa. | | | |
| O14520 | Aquaporin 7 (Aquaporin-7 like) | 1 . . . 346 | 317/346 (91%) | 0.0 |
| | (Aquaporin adipose) (AQPap) - | 1 . . . 342 | 324/346 (93%) | |
| | Homo sapiens (Human), 342 aa. | | | |
| O54794 | Aquaporin 7 - Mus musculus | 16 . . . 296 | 201/281 (71%) | e−114 |
| | (Mouse), 303 aa. | 1 . . . 277 | 233/281 (82%) | |
| P56403 | Aquaporin 7 - Rattus norvegicus | 20 . . . 285 | 188/266 (70%) | e−107 |
| | (Rat), 269 aa. | 4 . . . 265 | 220/266 (82%) | |
| P47862 | Aquaporin 3 (31.4 kDa water channel | 36 . . . 292 | 120/258 (46%) | 2e−68 |
| | protein) - Rattus norvegicus (Rat), | 20 . . . 277 | 175/258 (67%) | |
| | 292 aa. | | | |

PFam analysis indicates that the NOV41a protein contains the domains shown in the Table 41E.

TABLE 41E
Domain Analysis of NOV41a

| | | Identities/Similarities | |
|---|---|---|---|
| | NOV41a | for the | Expect |
| Pfam Domain | Match Region | Matched Region | Value |
| MIP: domain 1 of 1 | 31 . . . 276 | 78/268 (29%) | 5.2e−56 |
| | | 180/268 (67%) | |

### Example 42

The NOV42 clone was analyzed, and the nucleotide and polypeptide sequences are shown in Table 42A.

TABLE 42A
NOV42 Sequence Analysis

| | SEQ ID NO:175 | 895 bp |
|---|---|---|
| NOV42a, | CTGTCGTGGTTGTGGAATGCAAACGCCAGCACATAATGGAAACAGGACCTGAAGACCC | |
| CG56162-02 DNA | TTCCAGCATGCCAGAGGAAAGTTCCCCCAGGCGGACCCCGCAGAGCATTCCCTACCAG | |
| Sequence | GACCTCCCTCACCTGGTCAATGCAGACGGACAGTACCTCTTCTGCAGGTACTGGAAAC | |
| | CCACAGGCACACCCAAGGCCCTCATCTTTGTGTCCCATGGAGCCGGAGAGCACAGTGG | |
| | CCGCTATGAAGAGCTGGCTCGGATGCTGATGGGGCTGGACCTGCTGGTGTTCGCCCAC | |
| | GACCATGTTGGCCACGGACAGAGCGAAGGGGAGAGGATGGTAGTGTCTGACTTCCACG | |
| | TTTTCGTCAGGGATGTGTTGCAGCATGTGCATTCCATGCAGAAAGACTACCCTGGGCT | |
| | TCCTGTCTTCCTTCTGGGCCACTCCATGGGAGGCGCCATCGCCATCCTCACGGCCGCA | |
| | GAGAGGCCGGGCCACTTCGCCGGCATGGTACTCATTTCGCCTCTGGTTCTTGCCAATC | |
| | CTGAATCTGCAACAACTTTCAAGGTCGACATTTATAACTCAGACCCCCTGATCTGCCG | |
| | GGCAGGGCTGAAGGTGTGCTTCGGCATCCAACTGCTGAATGCCGTCTCACGGGTGGAG | |
| | CGCGCCCTCCCCAAGCTGACTGTGCCCTTCCTGCTGCTCCAGGGCTCCGCCGATCGCC | |
| | TATGTGACAGCAAAGGGGCCCACCTGCTCATGGAGTTAGCCAACAGCCAGGACAAGAC | |
| | TCTCAAGATTTATGAAGGTGCCTACCATGTTCTCCACAAGGAGCTTCCTGAAGTCACC | |
| | AACTCCGTCTTCCATGAAATAAACATGTGGGTCTCTCAAAGGACAGCCACGGCAGGAA | |
| | CTGCGTCCCCACCCTGAATGCATTG | |
| | ORF Start: ATG at 36 | ORF Stop: TGA at 885 |
| | SEQ ID NO:176 | 283 aa MW at 31122.4 kD |
| NOV42a, | METGPEDPSSMPEESSPRRTPQSIPYQDLPHLVNADGQYLFCRYWKPTGTPKALIFVS | |
| CG56162-02 Protein | HGAGEHSGRYEELARMLMGLDLLVFAHDHVGHGQSEGERMVVSDFHVFVRDVLQHVDS | |
| Sequence | MQKDYPGLPVFLLGHSMGGAIAILTAAERPGHFAGMVLISPLVLANPESATTFKVDIY | |
| | NSDPLICRAGLKVCFGIQLLNAVSRVERALPKLTVPFLLLQGSADRLCDSKGAHLLME | |
| | LAKSQDKTLKIYEGAYHVLHKELPEVTNSVFHEINMWVSQRTATAGTASPP | |
| | SEQ ID NO:177 | 861 bp |
| NOV42b, | AGATCTATGGAACAGGACCTGAAGACCCTTCCAGCATGCCAGAGGAAAGTTCCCCCA | |
| 174228465 DNA | GGCGGACCCCGCAGAGCATTCCCTACCAGGACCTCCCTCACCTGGTCAATGCAGACGG | |
| Sequence | ACAGTACCTCTTCTGCAGGTACTGGAAACCCACAGGCACACCCAAGGCCCTCATCTTT | |
| | GTGTCCCATGGAGCCGGAGAGCACAGTGGCCGCTATGAAGAGCTGGCTCGGATGCTGA | |
| | TGGGGCTGGACCTGCTGGTGTTCGCCCACGACCATGTTGGCCACGGACAGAGCGAAGG | |
| | GGAGAGGATGGTAGTGTCTGACTTCCACGTTTTCGTCAGGGATGTGTTGCAGCATGTG | |
| | GATTCCATGCAGAAAGACTACCCTGGGCTTCCTGTCTTCCTTCTGGGCCACTCCATGG | |
| | GAGGCGCCATCGCCATCCTCACGGCCGCAGAGAGGCCGGGCCACTTCGCCGGCATGGT | |
| | ACTCATTTCGCCTCTGGTTCTTGCCAATCCTGAATCTGCAACAACTTTCAAGGTCGAC | |
| | ATTTATAACTCAGACCCCCTGATCTGCCGGGCAGGGCTGAAGGTGTGCTTCGGCATCC | |
| | AACTGCTGAATGCCGTCTCACGGGTGGAGCGCGCCCTCCCCAAGCTGACTGTGCCCTT | |
| | CCTGCTGCTCCAGGGCTCTGCCGATCGCCTATGTGACAGCAAAGGGGCCTACCTGCTC | |
| | ATGGAGTTAGCCAAGAGCCAGGACAAGACTCTCAAGATTTATGAAGGTGCCTACCATG | |
| | TTCTCCACAAGGAGCTTCCTGAAGTCACCAACTCCGTCTTCCATGAAATAAACATGTG | |
| | GGTCTCTCAAAGGACAGCCACGGCAGGAACTGCGTCCCCACCCCTCGAG | |
| | ORF Start: AGA at 1 | ORF Stop: 8 at 862 |
| | SEQ ID NO:178 | 287 aa MW at 31634.0 kD |

| | |
|---|---|
| NOV42b, | RSMETGPEDPSSMPEESSPRRTPQSIPYQDLPHLVNADGQYLFCRYWKPTGTPKALIF |
| 174228465 Protein | VSHGAGEHSGRYEELARMLMGLDLLVFAHDHVGHGQSEGERMVVSDFHVFVRDVLQHV |
| Sequence | DSMQKDYPGLPVFLLGHSMGGAIAILTAAERPGHFAGMVLISPLVLANPESATTFKVD |
| | IYNSDPLICRAGLKVCFGIQLLNAVSRVERALPKLTVPFLLLQGSADRLCDSKGAYLL |
| | MELAKSQDKTLKIYEGAYHVLHKELPEVTNSVFHEINMWVSQRTATAGTASPPLE |

Sequence comparison of the above protein sequences yields the following sequence relationships shown in Table 42B.

TABLE 42B
Comparison of NOV42a against NOV42b and NOV42c.

| | NOV42a Residues/ | Identities/Similarities |
|---|---|---|
| Protein Sequence | Match Residues | for the Matched Region |
| NOV42b | 1 . . . 283 | 269/283 (95%) |
| | 3 . . . 285 | 270/283 (95%) |

Further analysis of the NOV42a protein yielded the following properties shown in Table 42C.

TABLE 42C
Protein Sequence Properties NOV42a

| | |
|---|---|
| PSort | 0.4500 probability located in cytoplasm; |
| analysis: | 0.4273 probability located in microbody (peroxisome); |
| | 0.2034 probability located in lysosome (lumen); |
| | 0.1000 probability located in mitochondrial matrix space |
| SignalP | No Known Signal Sequence Indicated |
| analysis: | |

A search of the NOV42a protein against the Geneseq database, a proprietary database that contains sequences published in patents and patent publication, yielded several homologous proteins shown in Table 42D.

TABLE 42D
Geneseq Results for NOV42a

| | | NOV42a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAG10770 | Arabidopsis thaliana protein fragment | 37 . . . 273 | 86/271 (31%) | 9e−27 |
| | SEQ ID NO: 9224 - Arabidopsis | 104 . . . 371 | 126/271 (45%) | |
| | thaliana, 383 aa. [EP1033405-A2, 06- | | | |
| | SEP-2000] | | | |
| AAG10769 | Arabidopsis thaliana protein fragment | 37 . . . 273 | 86/271 (31%) | 9e−27 |
| | SEQ ID NO: 9223 - Arabidopsis | 111 . . . 378 | 126/271 (45%) | |
| | thaliana, 390 aa. [EP1033405-A2, 06- | | | |
| | SEP-2000] | | | |
| AAG10768 | Arabidopsis thaliana protein fragment | 37 . . . 273 | 86/271 (31%) | 9e−27 |
| | SEQ ID NO: 9222 - Arabidopsis | 140 . . . 407 | 126/271 (45%) | |
| | thaliana, 419 aa. [EP1033405-A2, 06- | | | |
| | SEP-2000] | | | |
| AAG16436 | Arabidopsis thaliana protein fragment | 31 . . . 279 | 80/286 (27%) | 6e−24 |
| | SEQ ID NO: 17083 - Arabidopsis | 11 . . . 296 | 133/286 (45%) | |
| | thaliana, 301 aa. [EP1033405-A2, 06- | | | |
| | SEP-2000] | | | |
| AAG46122 | Arabidopsis thaliana protein fragment | 34 . . . 275 | 83/281 (29%) | 2e−23 |
| | SEQ ID NO: 57993 - Arabidopsis | 15 . . . 295 | 132/281 (46%) | |
| | thaliana, 351 aa. [EP1033405-A2, 06- | | | |
| | SEP-2000] | | | |

In a BLAST search of public sequence databases, the NOV42a protein was found to have homology to the proteins shown in the BLASTP data in Table 42E.

TABLE 42E
Public BLASTP Results for NOV42a

| | | NOV42a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q99685 | LYSOPHOSPHOLIPASE HOMOLOG | 1 . . . 283 | 282/313 (90%) | e−160 |
| | (LYSOPHOSPHOLIPASE-LIKE) - | 1 . . . 313 | 283/313 (90%) | |
| | Homo sapiens (Human), 313 aa. | | | |
| Q96AA5 | MONOGLYCERIDE LIPASE (EC | 11 . . . 283 | 272/303 (89%) | e−154 |
| | 3.1.1.23) - Homo sapiens (Human), | 1 . . . 303 | 273/303 (89%) | |
| | 303 aa. | | | |
| O35678 | MONOGLYCERIDE LIPASE (EC | 11 . . . 283 | 230/303 (75%) | e−133 |
| | 3.1.1.23) - Mus musculus (Mouse), 303 | 1 . . . 303 | 255/303 (83%) | |
| | aa. | | | |
| O11456 | H14-E - Ectromelia virus, 277 aa. | 32 . . . 273 | 122/270 (45%) | 6e−64 |
| | | 6 . . . 274 | 168/270 (62%) | |
| P87627 | 41KBP FRAGMENT FROM LEFT | 32 . . . 273 | 121/270 (44%) | 5e−63 |
| | END OF GENOME - Cowpox virus | 6 . . . 274 | 167/270 (61%) | |
| | (CPV), 276 aa. | | | |

PFam analysis indicates that the NOV42a protein contains the domains shown in the Table 42F.

TABLE 42F
Domain Analysis of NOV42a

| | | Identities/ | |
|---|---|---|---|
| | NOV42a Match | Similarities for | Expect |
| Pfam Domain | Region | the Matched Region | Value |
| abhydrolase: domain | 80 . . . 270 | 46/247 (19%) | 0.033 |
| 1 of 1 | | 134/247 (54%) | |
| Thioesterase: domain | 53 . . . 270 | 47/271 (17%) | 1.7 |
| 1 of 1 | | 139/271 (51%) | |

### Example 43

The NOV43 clone was analyzed, and the nucleotide and polypeptide sequences are shown in Table 43A.

TABLE 43A
NOV43 Sequence Analysis

| | SEQ ID NO:179 | 2018 bp |
|---|---|---|
| NOV43a, | GCGCCCCCCACCCTTCCGGCCCCCCAGAACCCGCGCCATCCCCCGGAGCCTCCCCAGA | |
| CG59681-01 DNA | GCTGGCCGCGCAGGATGGGGCGCCCTCAGGCCCACGCTGCTGCCGCCTTCGCTGCCGCT | |
| Sequence | GCTGCTGCTCCTAATGCTAGGAATGGGATGCTGGGCCCGGGAGGTGCTGGTCCCCGAG | |
| | GGGCCCTTGTACCGCGTGGCTGGCACAGCTGTCTCCATCTCCTGCAATGTGACCGGCT | |
| | ATGAGGGCCCTGCCCAGCAGAACTTCCAGTGGTTCCTCTATAGGCCCGAGGCCCCAGA | |
| | TACTGCACTGGGCATTGTCAGTACCAAGGATACCCAGTTCTCCTATGCTGTCTTCAAG | |
| | TCCCCAGTGGTGGCCCGTGAGGTGCAGGTGCAGCGCCTACAAGGTGATGCCGTGGTGC | |
| | TCAAGATTGCCCCCCTGCAGGCCCAGGATGCCGGCATTTATGAGTGCCACACCCCCTC | |
| | CACTGATACCCGCTACCTGGGCAGCTACAGCGGCAAGGTGGAGCTGAGAGTTCTTCCA | |
| | GATGTCCTCCAGGTGTCTGCTGCCCCCCCAGGGCCCCGAGGCCGCCAGGCCCCAACCT | |
| | CACCCCCACGCATGACGGTGCATCAGGGGCAGGAGCTGGCACTGGGCTGCCTGGCGAG | |
| | GACAAGCACACAGAAGCACACACACCTGGCAGTGTCCTTTGGGCGATCTGTGCCCGAG | |
| | GCACCAGTTGGGCGGTCAACTCTGCAGGAAGTGGTGGGAATCCGGTCAGACTTGGCCG | |
| | TGGAGGCTGGAGCTCCCTATGCTGAGCGATTCGCTGCAGGGGAGCTTCGTCTGGGCAA | |
| | GGAAGGGACCGATCGGTACCGCATGGTAGTAGGGGGTGCCCAGGCAGGGGACGCAGGC | |
| | ACCTACCACTGCACTGCCGCTGAGTGGATTCAGGATCCTGATGGCAGCTGGGCCCAGA | |
| | TTGCAGAGAAAAGGGCCGTCCTGGCCCACGTGGATGTGCAGGCACTGTCCAGCCAGCT | |
| | GGCAGTGACAGTGGGGCCTGGTGAACGTCGGATCGGCCCAGGGGAGCCCTTGGAACTG | |
| | CTGTGCAATGTGTCAGGGGCACTTCCCCCAGCAGGCCGTCATGCTGCATACTCTGTAG | |
| | GTTGGGAGATGGCACCTGCCGGGGCACCTGGGCCCGGCCGCCTGGTAGCCCAGCTGGA | |
| | CACAGAGGGTGTGGGCAGCCTGGGCCCTGGCTATGAGGGCCGACACATTGCCATGGAG | |
| | AAGGTGGCATCCAGAACATACCGGCTACGGCTAGAGGCTCCCACGCCTGGTGATGCGG | |
| | GCACCTACCGCTGCCTCGCCAAAGCCTATGTTCGAGGGTCTGGGACCCGGCTTCGTGA | |
| | AGCAGCCAGTGCCCGTTCCCGGCCTCTCCCTGTACATGTGCGGGAGGAAGGTGTGGTG | |
| | CTGGAGGCTGTGGCATGGCTAGCAGGAGGCACAGTGTACCGCGGGGAGACTGCCTCCC | |
| | TGCTGTGCAACATCTCTGTGCGGGGTGGCCCCCCAGGACTGCGGCTGGCCGCCAGCTG | |
| | GTGGGTGGAGCGACCAGAGGACGGAGAGCTCAGCTCTGTCCCTGCCCAGCTGGTGGGT | |
| | GGCGTAGGCCAGGATGGTGTGGCAGAGCTGGGAGTCCGGCCTGGAGGAGGCCCTGTCA | |
| | GCGTAGAGCTGGTGGGGCCCCGAAGCCATCGGCTGAGACTACACAGCTTGGGGCCCGA | |
| | GGATGAAGGCGTGTACCACTGTGCCCCCAGCGCCTGGGTGCAGCATGCCGACTACAGC | |
| | TGCTACCAGGCGGGCAGTGCCCGCTCAGGGCCTGTTACAGTCTACCCCTACATGCATG | |
| | GTGAGTGACACCCCCTCCACCCTCCTCACTCTGCCTTCCTCCTGGCCTCTGCCACTGG | |
| | CCTTCCCTTCCCATCTTCTGACCCTCCTGCTACTATCTCTCTCCTCCACATTATGTCA | |
| | CATCAACTCTCAAAAAATCCAACTTCCAGCCCTGCAGTGCCCACCTGCACGGGGTCCT | |
| | CTGTGGTTGATGCTGACTTGCATGCTGAGGGTGCATGGTGGGCAGC | |
| | ORF Start: ATG at 73 | ORF Stop: TGA at 1804 |
| | SEQ ID NO:180 | 577 aa MW at 61171.8 kD |
| NOV43a, | MCALRPTLLPPSLPLLLLLMLGMCCWAREVLVPEGPLYRVAGTAVSISCNVTGYEGPA | |
| CG59681-01 Protein | QQNFEWFLYRPEAPDTALGIVSTKDTQFSYAVFKSRVVAGEVQVQRLQGDAVVLKIAR | |
| Sequence | LQAQDAGIYECHTPSTDTRYLGSYSGKVELRVLPDVLQVSAAPPGPRGRQAPTSPPRM | |
| | TVHEGQELALGCLARTSTQKHTHLAVSFGRSVPEAPVGRSTLQEVVGIRSDLAVEAGA | |
| | PYAERLAAGELRLGKEGTDRYRMVVGGAQAGDAGTYHCTAAEWIQDPDGSWAQIAEKR | |
| | AVLAHVDVQALSSQLAVTVGPGERRIGPGEPLELLCNVSGALPPAGRHAAYSVGWEMA | |
| | PAGAPGPGRLVAQLDTEGVGSLGPGYEGRHIAMEKVASRTYRLRLEAARPGDAGTYRC | |
| | LAKAYVRGSGTRLREAASARSRPLPVHVREEGVVLEAVAWLAGGTVYRGETASLLCNI | |
| | SVRGGPPGLRLAASWWVERPEDGELSSVPAQLVGGVGQDGVAELGVRPGGGPVSVELV | |
| | GPRSHRLRLHSLCPEDEGVYHCAPSAWVQHADYSWYQACSARSGPVTVYPYMHGE | |
| | SEQ ID NO:181 | 1662 bp |
| NOV43b, | AGATCTCGGGAGGTGCTGGTCCCCGAGGGGCCCTTGTACCGCGTCGCTGGCACAGCTG | |
| 174308213 DNA | TCTCCATCTCCTGCAATGTGACCGGCTATGAGGGCCCTGCCCAGCAGAACTTCGAGTG | |
| Sequence | GTTCCTGTATAGGCCCGAGGCCCCAGATACTGCACTGGGCATTGTCAGTACCAAGGAT | |
| | ACCCAGTTCTCCTATGCTGTCTTCAAGTCCCGAGTGGTGGCGGGTGAGGTGCAGGTGC | |
| | AGCGCCTACAAGGTGATGCCGTGGTGCTCAAGATTGCCCGCCTGCAGGCCCAGGATGC | |
| | CGGCATTTATGAGTGCCACACCCCCTCCACTGATACCCGCTACCTGGGCAGCTACAGC | |
| | GGCAAGGTGCAGCTGAGAGTTCTTCCAGATCTCCTCCAGGTGTCTGCTCCCCCCCCAG | |
| | GGCCCCGAGGCCGCCAGGCCCCAACCTCACCCCCACGCATGACGGTGCATGAGGGGCA | |
| | GGAGCTGGCACTGGGCTGCCTGGCGAGGACAAGCACACAGAAGCACACACACCTGGCA | |
| | GTGTCCTTTGGGCGATCTCTGCCCGAGGCACCAGTTGGGCGGTCAACTCTGCAGGAAG | |
| | TGGTGGGAATCCGGTCAGACTTGGCCGTGGAGGCTGGAGCTCCCTATGCTGAGCGATT | |
| | GGCTGCAGGGGAGCTTCGTCTGGGCAAGGAAGGGACCGATCCGTACCCCATGGTAGTA | |
| | GGGGGTCCCCAGGCAGGGCACGCAGGCACCTACCACTGCACTGCCGCTGAGTGGATTC | |
| | AGGATCCTGATGGCAGCTGGGCCCAGATTGCAGAGAAAAGGGCCGTCCTGGCCCACGT | |
| | GGATGTGCAGACGCTGTCCAGCCAGCTGGCAGTGACAGTGGGGCCTGGTGAACGTCGG | |
| | CAGGCCGTCATGCTGCATACTCTGTAGGTTGGGAGATGGCACCTGCGGGGGCACCTGG | |
| | GCCCGGCCGCCTGGTAGCCCAGCTGGACACAGAGGGTGTGGGCAGCCTGGGCCCTGGC | |
| | TATGAGGGCCGACACATTGCCATGGAGAAGGTGGCATCCAGAACATACCGGCTACGGC | |
| | TAGAGGCTGCCAGGCCTGGTGATGCGGGCACCTACCGCTGCCTCCCCAAAGCCTATGT | |
| | TCGAGGGTCTGGGACCCGGCTTCGTGAAGCAGCCAGTGCCCGTTCCCGGCCTCTCCCT | |
| | GTACATGTGCGGGAGGAAGGTGTGGTGCTGGAGGCTGTGGCATGGCTAGCAGGAGGCA | |
| | CAGTGTACCGCGGGGAGACTGCCTCCCTGCTGTGCAACATCTCTGTGCGGGGTGGCCC | |
| | CCCAGGACTGCGGCTGGCCGCCAGCTGGTGGGTGGAGCGACCAGAGGACGGAGAGCTC | |
| | AGCTCTGTCCCTGCCCAGCTGGTGGGTGGCGTAGGCCAGGATGGTGTGGCAGAGCTGG | |
| | GAGTCCGGCCTGCAGGAGGCCCTGTCAGCGTAGAGCTGGTGGGGCCCCGAAGCCATCG | |
| | GCTGAGACTACACAGCTTGGGGCCCGAGGATGAAGGCGTGTACCACTGTGCCCCCAGC | |
| | GCCTGGGTGCAGCATGCCGACTACAGCTGGTACCAGGCGGGCAGTGCCCGCTCAGGGC | |
| | CTGTTACAGTCTACCCCTATATGCATGGTGAGCTCGAG | |
| | ORF Start: AGA at 1 | ORF Stop: A at 1663 |
| | SEQ ID NO:182 | 554 aa MW at 58826.7 kD |
| NOV43b, | RSREVLVPEGPLYRVAGTAVSISCNVTGYEGPAQQNFEWFLYRPEAPDTALGIVSTKD | |
| 174308213 Protein | TQFSYAVFKSRVVAGEVQVQRLQGDAVVLKIARLQAQDAGIYECHTPSTDTRYLGSYS | |
| Sequence | GKVELRVLPDVLQVSAAPPGPRGRQAPTSPPRMTVHEGQELALGCLARTSTQKHTHLA | |
| | VSFGRSVPEAPVGRSTLQEVVGIRSDLAVEAGAPYAERLAAGELRLGKEGTDRYRMVV | |
| | GGAQAGDAGTYHCTAAEWIQDPDGSWAQIAEKRAVLAHVDVQTLSSQLAVTVGPGERR | |
| | IGPGEPLELLCNVSGALPPAGRHAAYSVGWEMAPAGAPGPGRLVAQLDTEGVGSLGPG | |
| | YEGRHIAMEKVASRTYRLRLEAARPGDAGTYRCLAKAYVRGSGTRLREAASARSRPLP | |
| | VHVREEGVVLEAVAWLAGGTVYRGETASLLCNISVRGGPPCLRLAASWWVERPEDGEL | |
| | SSVPAQLVGGVGQDGVAELGVRPGGGPVSVELVGPRSHRLRLHSLGPEDEGVYHCAPS | |
| | AWVQHADYSWYQAGSARSGPVTVYPYMHGELE | |
| | SEQ ID NO:183 | 1662 bp |
| NOV43c, | AGATCTCCGGAGGTGCTGGTCCCCGAGGGGCCCTTGTACCGCGTGGCTGGCACAGCTG | |
| 174308218 DNA | TCTCCATCTCCTGCAATGTGACCGGCTATGAGGGCCCTGCCCAGCAGAACTTCGAGTG | |
| Sequence | CTTCCTGTATAGGCCCGAGGCCCCACATACTCCACTGCGCATTGTCACTACCAAGGAT | |
| | ACCCAGTTCTCCTATGCTGTCTTCAAGTCCCGAGTGGTGGCGGGTGAGGTGCAGGTGC | |
| | AGCGCCTACAAGGTGATGCCGTGGTGCTCAAGATTGCCCGCCTGCAGGCCCAGGATGC | |
| | CGGCATTTATGAGTGCCACACCCCCTCCACTGATACCCGCTACCTGGGCAGCTACAGC | |
| | GGCAAGGTGGAGCTGAGAGTTCTTCCAGATGTCCTCCAGGTGTCTGCTGCCCCCCCAG | |
| | GGCCCCGAGGCCGCCAGGCCCCAACCTCACCCCCACGCATGACGGTGCATGAGGGGCA | |
| | GGAGCTGGCACTGGGCTGCCTGGCGAGGACAAGCACACAGAAGCACACACACCTGGCA | |
| | GTGTCCTTTGGGCGATCTGTGCCCGAGGCACCAGTTGGGCGGTCAACTCTGCAGGAAG | |
| | TGGTGGGAATCCGGTCAGACTTGGCCGTGCAGGCTGGAGCTCCCTATGCTGAGCGATT | |
| | GGCTGCAGGGGAGCTTCGTCTGCGCAAGGAAGGCACCGATCGGTACCGCATGGTAGTA | |
| | GGGGGTGCCCAGGCAGGGGACGCAGGCACCTACCACTGCACTGCCGCTGAGTGGATTC | |
| | AGGATCCTGATGGCAGCTGGGCCCAGATTGCAGAGAAAAGGGCCGTCCTGGCCCACGT | |
| | GGATGTGCAGACGCTGTCCAGCCAGCTGGCAGTGACAGTGGGGCCTGGTGAACGTCGG | |
| | ATCGGCCCAGGGGACCCCTTGGAACTGCTGTGCAATGTGTCAGGGGCACTTCCCCCAG | |
| | CAGGCCGTCATGCTGCATACTCTGTAGGTTGGGAGATGGCACCTGCGGGGGCACCTGG | |
| | GCCCGGCCGCCTGGTAGCCCAGCTGGACACAGAGGGTGTGGGCAGCCTGGGCCCTGGC | |
| | TATGAGGCCCGACACATTCCCATGGAGAAGGTGGCATCCAGAACATACCGGCTACGGC | |
| | TAGAGGCTGCCAGGCCTGGTGATGCGGGCACCTACCGCTGCCTCGCCAAAGCCTATGT | |
| | TCGAGGGTCTGGGACCCGGCTTCGTGAAGCAGCCAGTGCCTGTTCCCGCCCTCTCCCT | |
| | GTACATGTGCGGGAGGAAGGTGTGGTGCTGGAGGCTGTGGCATGGCTAGCAGGAGGCA | |
| | CAGTGTACCGCGGGGAGACTGCCTCCCTGCTGTGCAACATCTCTGTGCGGGGTGCCCC | |
| | CCCAGGACTGCGGCTGGCCOCCAGCTGGTGGGTGGAGCGACCAGAGGACGGAGAGCTC | |
| | AGCTCTGTCCCTGCCCAGCTGGTGGGTGGCGTAGGCCAGGATGGTGTGGCAGAGCTGG | |
| | GAGTCCGGCCTGGAGGAGGCCCTGTCAGCGTAGAGCTGGTGGGGCCCCGAAGCCATCG | |
| | GCTGAGACTACACAGCTTGGGGCCCGAGGATGAAGGCGTGTACCACTGTCCCCCCAGC | |
| | GCCTGGGTGCAGCATGCCGACTACAGCTGGTACCAGGCGGGCAGTGCCCGCTCAGGGC | |
| | CTGTTACAGTCTACCCCTACATGCATCGTGAGCTCGAG | |
| | ORF Start: AGA at 1 | ORF Stop: A at 1663 |
| | SEQ ID NO:184 | 554 aa MW at 58773.7 kD |

| | | |
|---|---|---|
| NOV43c, | RSREVLVPEGPLYRVAGTAVSISCNVTGYEGPAQQNFEWFLYRPEAPDTALGIVSTKD | |
| 1743082 18 Protein | TQFSYAVFKSRVVAGEVQVQRLQGDAVVLKIARLQAQDAGIYECHTPSTDTRYLGSYS | |
| Sequence | | GKVELRVLPDVLQVSAAPPGPRGRQAPTSPPRMTVHEGQELALGCLARTSTQKHTHLA |
| | VSFGRSVPEAPVGRSTLQEVVGIRSDLAVEAGAPYAERLAAGELRLGKEGTDRYRMVV | |
| | GGAQAGDAGTYHCTAAEWIQDPDGSWAQIAEKRAVLAHVDVQTLSSQLAVTVGPGERR | |
| | IGPGEPLELLCNVSGALPPAGRHAAYSVGWEMAPAGAPGPGRLVAQLDTEGVGSLGPG | |
| | YEGRHIAMEKVASRTYRLRLEAARPGDAGTYRCLAKAYVRGSGTRLREAASACSRPLP | |
| | VHVREEGVVLEAVAWLAGGTVYRGETASLLCNISVRGGPPGLRLAASWWVERPEDGEL | |
| | SSVPAQLVGGVGQDGVAELGVRPGGGPVSVELVGPRSHRLRLHSLGPEDEGVYHCAPS | |
| | AWVQHADYSWYQAGSARSGPVTVYPYMHGELE | |

| | SEQ ID NO:185 | 11662 bp |
|---|---|---|
| NOV43d, | AGATCTCGGGAGGTGCTGGTCCCCGAGGGGCCCTTGTACCGCGTGGCTGGCACAGCTG | |
| 174308224 DNA | TCTCCATCTCCTGCAATGTGACCGGCTATGAGGGCCCTGCCCACCAGAACTTCGAGTG | |
| Sequence | GTTCCTGTATAGGCCCGAGGCCCCAGATACTGCACTGGGCATTGTCAGTACCAAGGAT | |
| | ACCCAGTTCTCCTATGCTGTCTTCAAGTCCCGAGTGGTGGCGGGTGAGGTGCAGGTGC | |
| | AGCGCCTACAAGGTGATGCCGTGGTCCTCAAGATTGCCCGCCTGCAGGCCCAGGATGC | |
| | CGGCATTTATGAGTGCCACACCCCCTCCACTGATACCCGCTACCTGGGCAGCTACAGC | |
| | GGCAAGGTGGAGCTGAGAGTTCTTCCAGATGTCCTCCAGGTGTCTGCTGCCCCCCCAG | |
| | GGCCCCGAGGCCGCCAGOCCCCAACCTCACCCCCACGCATGACGGTGCATGAGGCGCA | |
| | GGAGTTGGCACTGGGCTGCCTGCCGAGGACAAGCACACAGAAGCACACACACCTGGCA | |
| | GTGTCCTTTGGGCGATCTGTGCCCGAGCCACCAGTTGGGCGGTCAACTCTGCAGGAAG | |
| | TGGTGGGAATCCGGTCAGACTTGGCCGTGGAGGCTGGAGCTCCCTATGCTGAGCGATT | |
| | GGCTGCAGGGGAGCTTCGTCTGGGCAAGGAAGGGACCGATCGGTACCGCATGGTAGTA | |
| | GGGGGTGCCCAGGCAGGGGACGCAGGCACCTACCACTGCACTGCCGCTGAOTGGATTC | |
| | AGGATCCTGATGGCAGCTGGGCCCAGATTGCAGAGAAAAGGGCCGTCCTCGCCCACGT | |
| | GGATGTGCAGACGCTGTCCAGCCAGCTGGCAGTGACAGTGGGGCCTGGTGAACGTCGG | |
| | ATCGGCCCAGGGGAGCCCTTGGAACTGCTGTGCAATGTGTCAGGGGCACTTCCCCCAG | |
| | CAGGCCGTCATGCTGCATACTCTCTAGGTTGGGAGATGGCACCTGCGGGGGCACCTGG | |
| | GCCCGGCCGCCTGGTAGCCCAGCTGGACACAGAGGGTGTGCGCAGCCTGGGCCCTGGC | |
| | TATGAGGGCCGACACATTGCCATGGAGAAGGTGGCATCCAGAACATACCGGCTACGGC | |
| | TAGAGGCTGCCAGGCCTGGTGATGCGGGCACCTACCGCTGCCTCGCCAAACCCTATGT | |
| | TCGAGGGTCTGGGACCCGGCTTCGTGAAGCAGCCAGTGCCCGTTCCCGGCCTCTCCCT | |
| | GTACATGTGCGGGAGGAAGGTGTGGTGCTGGAGGCTGTGGCATGGCTAGCAGGAGGCA | |
| | CAGTGTACCGCGGGGAGACTGCCTCCCTGCTGTGCAACATCTCTGTGCGGGGTGGCCC | |
| | CCCAGGACTGCGGCTGGCCGCCAGCTGGTGGGTGGAGCGACCAGAGGACCGAGAGCTC | |
| | AGCTCTGTCCCTGCCCAGCTGGTGGGTGGCGTAGGCCAGGATGGTGTGGCAGAGCTGG | |
| | GAGTCCGGCCTGGAGGAGGCCCTGTCAGCGTAGAGCTGGTGGGGCCCCGAAGCCATCG | |
| | GCTGAGACTACACAGCTTGGGACCCGAGGATGAAGGCGTGTACCACTGTGCCCCCAGC | |
| | GCCTGGGTGCAGCATGCCGACTACAGCTGGTACCAGGCGGGCAGTGCCCGCTCAGGGC | |
| | CTGTTACAGTCTACCCCTACATGCATGGTGAGCTCGAG | |
| | ORF Start: AGA at 1 | ORF Stop: A at 1663 |
| | SEQ ID NO:186 | 554 aa MW at 58826.7 kD |

| | |
|---|---|
| NOV43d, | RSREVLVPEGPLYRVAGTAVSISCNVTGYEGPAQQNFEWFLYRPEAPDTALGIVSTKD |
| 174308224 Protein | TQFSYAVFKSRVVAGEVQVQRLQGDAVVLKIARLQAQDAGIYECHTPSTDTRYLGSYS |
| Sequence | GKVELRVLPDVLQVSAAPPGPRGRQAPTSPPRMTVHEGQELALGCLARTSTQKHTHLA |
| | VSFGRSVPEAPVGRSTLQEVVGIRSDLAVEAGAPYAERLAAGELRLCKEGTDRYRMVV |
| | GGAQAGDAGTYHCTAAEWIQDPDGSWAQIAEKRAVLAHVDVQTLSSQLAVTVGPGERR |
| | IGPGEPLELLCNVSGALPPAGRHAAYSVGWEMAPAGAPGPGRLVAQLDTEGVGSLGPG |
| | YEGRHIANEKVASRTYRLRLEAARPGDACTYRCLAKAYVRGSGTRLREAASARSRPLP |
| | VHVREEGVVLEAVAWLAGGTVYRGETASLLCNISVRGGPPGLRLAASWWVERPEDGEL |
| | SSVPAQLVGGVGQDGVAELGVRPGGGPVSVELVGPRSHRLRLHSLGPEDEGVYHCAPS |
| | AWVQHADYSWYQAGSARSGPVTVYPYMHGELE |

Sequence comparison of the above protein sequences yields the following sequence relationships shown in Table 43B.

TABLE 43B
Comparison of NOV43a against NOV43b through NOV43d.

| | NOV43a Residues/ | Identities/Similarities |
|---|---|---|
| Protein Sequence | Match Residues | for the Matched Region |
| NOV43b | 27 . . . 577 | 525/551 (95%) |
| | 2 . . . 552 | 526/551 (95%) |
| NOV43c | 27 . . . 577 | 524/551 (95%) |
| | 2 . . . 552 | 525/551 (95%) |
| NOV43d | 27 . . . 577 | 525/551 (95%) |
| | 2 . . . 552 | 526/551 (95%) |

Further analysis of the NOV43a protein yielded the following properties shown in Table 43C.

TABLE 43C
Protein Sequence Properties NOV43a

| | |
|---|---|
| PSort | 0.8497 probability located in lysosome (lumen); |
| analysis: | 0.5947 probability located in outside; |
| | 0.1197 probability located in microbody (peroxisome); |
| | 0.1000 probabilitylocated in endoplasmic |
| | reticulum (membrane) |
| SignalP | Cleavage site between residues 28 and 29 |
| analysis: | |

A search of the NOV43a protein against the Geneseq database, a proprietary database that contains sequences published in patents and patent publication, yielded several homologous proteins shown in Table 43D.

TABLE 43D
Geneseq Results for NOV43a

| | | NOV43a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAM24248 | Human EST encoded protein SEQ ID | 1 . . . 575 | 574/575 (99%) | 0.0 |
| | NO: 1773 - Homo sapiens, 613 aa. | 1 . . . 575 | 574/575 (99%) | |
| | [WO200154477-A2, 02-AUG-2001] | | | |
| AAB90544 | Human secreted protein, SEQ ID NO: | 1 . . . 575 | 574/575 (99%) | 0.0 |
| | 82 - Homo sapiens, 613 aa. | 1 . . . 575 | 574/575 (99%) | |
| | [WO200121658-A1, 29-MAR-2001] | | | |
| AAB15536 | Human immune system molecule from | 1 . . . 575 | 574/575 (99%) | 0.0 |
| | Incyte clone 2705028 - Homo sapiens, | 1 . . . 575 | 574/575 (99%) | |
| | 613 aa. [WO200060080-A2, | | | |
| | 12-OCT-2000] | | | |
| AAB90560 | Human secreted protein, SEQ ID NO: | 1 . . . 575 | 573/575 (99%) | 0.0 |
| | 98 - Homo sapiens, 613 aa. | 1 . . . 575 | 573/575 (99%) | |
| | [WO200121658-A1, 29-MAR-2001] | | | |
| AAB81411 | Partial human IgSF protein, SEQ ID | 136 . . . 575 | 437/440 (99%) | 0.0 |
| | NO: 5 - Homo sapiens, 478 aa. | 1 . . . 440 | 439/440 (99%) | |
| | [WO200127278-A2, 19-APR-2001] | | | |

In a BLAST search of public sequence databases, the NOV43a protein was found to have homology to the proteins shown in the BLASTP data in Table 43E.

TABLE 43E
Public BLASTP Results for NOV43a

| | | NOV43a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q969P0 | EWI2 (CD81 PARTNER 3) - | 1 . . . 575 | 574/575 (99%) | 0.0 |
| | Homo sapiens (Human), 613 aa. | 1 . . . 575 | 574/575 (99%) | |
| AAL02217 | IMMUNOGLOBULIN | 1 . . . 575 | 508/575 (88%) | 0.0 |
| | SUPERFAMILY RECEPTOR PGRL - | 1 . . . 573 | 542/575 (93%) | |
| | Mus musculus (Mouse), 611 aa. | | | |
| Q9BTG9 | PROTEIN - Homo sapiens (Human), | 4 . . . 449 | 445/446 (99%) | 0.0 |
| | 487 aa (fragment). | | | |
| CAC37841 | SEQUENCE 4 FROM PATENT | 136 . . . 575 | 437/440 (99%) | 0.0 |
| | WO0127278 - Homo sapiens | 1 . . . 440 | 439/440 (99%) | |
| | (Human), 478 aa (fragment). | | | |
| CAC37842 | SEQUENCE 6 FROM PATENT | 174 . . . 575 | 401/402 (99%) | 0.0 |
| | WO0127278 - Homo sapiens | 1 . . . 402 | 401/402 (99%) | |
| | (Human), 440 aa. | | | |

PFam analysis indicates that the NOV43a protein contains the domains shown in the Table 43F.

TABLE 43F
Domain Analysis of NOV43a

| | | Identities/ | |
|---|---|---|---|
| | | Similarities | |
| | NOV43a | for the | |
| Pfam Domain | Match Region | Matched Region | Expect Value |
| ig: domain 1 of 4 | 42 . . . 129 | 13/89 (15%) | 0.0011 |
| | | 55/89 (62%) | |
| ig: domain 2 of 4 | 179 . . . 272 | 8/97 (8%) | 0.74 |
| | | 58/97 (60%) | |
| ig: domain 3 of 4 | 319 . . . 408 | 15/91 (16%) | 0.00012 |
| | | 60/91 (66%) | |
| ig: domain 4 of 4 | 455 . . . 546 | 15/93 (16%) | 3.7e−05 |
| | | 61/93 (66%) | |

### Example 44

The NOV44 clone was analyzed, and the nucleotide and polypeptide sequences are shown in Table 44A.

TABLE 44A
NOV44 Sequence Analysis

| | SEQ ID NO:187 | 3201 bp |
|---|---|---|
| NOV44a, | AGAGTCCTCAGCAGGGTAGCCGAGCCCAGGCCACTTCTGCTGAGGATGGGGCAGGCTG | |
| CG59869-01 DNA | GGGTCTGGGTGTGGCCTGGGGTGGCTCAGGGCTGGAACTGCTGCCTGATTCCTGTGTG | |
| Sequence | GGGAGAAGCTCAGTGGCCGTTTCCTGCCACTGACAAGGATTTCACATGCAGAAGAGAA | |
| | AAGGCCCCCCTCCACCCCCCGCATTCCCTGCCGACTGAGAGCCAGTGTTTGCTGCCCT | |
| | TGCTGGGGGCGGGTAGGAAACCCTGAGCTTCCTGATGCGGAGTCATGAAGCAGAGTCC | |
| | TCGGGAAGGCATCTCCCCAACCTTCCCTCATCTCTGGCGGCCCTCTTGGGCCTCTGAC | |
| | CCAGCCCCTCCCCGGGCCAGGCTCACAGAAGCTGGCTTCTGGGACTGTCCTGGGCCCA | |
| | AGTGGGCACCTGCGCCAGCCCCACCTGTGCCTGGGCTGTGGCCCCTTCCTACAGGGCG | |
| | CTCACCATGGCCCCGCCGCTCCTGCTGCTGCTGCTGGCCAGTGGAGCGGCCGCCTGCC | |
| | CGCTGCCCTGCGTCTGCCAGAACCTGTCCGAGTCGCTCAGCACCCTCTGTGCCCACCG | |
| | AGGCCTGCTGTTTGTGCCGCCCAACGTGGACCGGCGCACAGTGGAGCTGCGGCTGGCT | |
| | GACAACTTCATCCAGGCCCTGGGGCCCCCTGACTTCCGCAACATGACGGGACTGGTGG | |
| | ACCTGACACTGTCTCGCAATGCCATCACCCGCATTGGGGCCCGCGCCTTTGGGGACCT | |
| | CGAGAGCCTGCGTTCCCTCCACCTTGACGGCAACAGGCTGGTGGAGCTGGGCACCGGG | |
| | ACCCTCCGGGGCCCCGTCAATCTGCAGCACCTCATCCTCAGCGGCAACCAGCTGGGCC | |
| | GCATCGCGCCGGGAGCCTTCCACGACTTCCTAGAGAGCCTGGAGGACCTGGACCTGTC | |
| | CTACAACAACCTCCGGCAGGTGCCCTGGGCCGGCATCGGCGCCATGCCTGCCCTGCAC | |
| | ACCCTCAACCTGGACCATAACCTTATTGACGCACTGCCCCCAGGCGCCTTCGCCCAGC | |
| | TCGGTCAGCTCTCCCGCCTGGACCTCACCTCCAACCGCCTGGCCACGCTGGCTCCGGA | |
| | CCCGCTTTTCTCTCGTGCGCGTCATGCAGAGGCCTCTCCCGCCCCCCTGGTGCTGAGC | |
| | TTTAGCGGGAACCCCCTGCACTGCAACTGTGAGCTGCTGTGGCTGCGGCGGCTGGCGC | |
| | GGCCGGACGACCTGGAAACGTGCGCCTCCCCGCCCCGCCTGGCCCGCCGCTACTTCTG | |
| | GGCAGTGCCCGAGGGCGAGTTCTCCTGTGAGCCGCCCCTCATTGCCCGCCACACGCAG | |
| | CGCCTCTGGGTGCTGGAAGGCCAGCGGGCCACGCTGCGGTGCCGGGCCCTGGGTGACC | |
| | CCGCGCCTACCATGCACTGGGTCGGTCCTGACGACCGGTTGGTTGGCAACTCCTCCCG | |
| | AGCCCGGGCTTTCCCCAACGCGACCTTAGAGATTGGGCTGACCGGCGCTCGGGACGCT | |
| | GGGGGCTACACCTGCATCGCCACCAACCCTGCTGGTGAGGCCACAGCCCGAGTAGAAC | |
| | TGCOGGTGCTGGCCTTGCCCCATGGTGGGAACAGCAGTGCCGAGGGGGGCCGCCCCGG | |
| | GCCCTCGGACATCGCCGCCTCCGCTCGCACTGCTCCCGAGGGTGAGGGGACGCTGGAG | |
| | TCTGAGCCAGCCGTGCAGGTGACGGAGGTGACCGCCACCTCAGGGCTGGTGAGCTGGG | |
| | GTCCCGGGCGGCCAGCCGACCCAGTGTGGATGTTCCAAATCCAGTACAACAGCACCGA | |
| | CACCTCGTCCCCGGCGCTGACTATGACCTCTGCCTGCTGGCCTTGTCACCGGCCGCTG | |
| | GGCCCTCTGACCTCACGGCCACCAGGCTGCTGGGCTGTGCCCATTTCTCCACGCTGCC | |
| | GGCCTCGCCCCTGTGCCACGCCCTGCAGGCCCACGTGCTGGGCGGGACCCTGACCGTG | |
| | GCCGTGGGGGGTGTGCTGGTGGCTGCCTTACTGGTCTTCACTGTGGCCTTGCTGGTTC | |
| | GGGGCCGGGGGGCCCGAAATGGCCGCCTCCCCCTCAAGCTCAGCCACGTCCAGTCCCA | |
| | GACCAATGGAGGCCCCAGCCCCACACCCAAGGCCCACCCGCCGCGGGAGCCCCCCGCC | |
| | CCGGCCGCAGCGCAGCTGCTCTCTGGACCTGGGAGATGCCGGGTGCTACGGTTATGCC | |
| | AGGCGCCTGGGAGGAGCTTCGGCCCGACCGAGCCACTCTGTGCATGGGGGGCTGCTCG | |
| | GGGCAGGGTGCCGGGGGGTAGGAGGCAGCGCCGAGCGGCTGGAAGAGAGTGTGGTGTG | |
| | ATGGACGGGCAGCTTCCTGTGTGCTCCAAGGGATGAGCCTCGTGGGGCAGAGGGCCCG | |
| | GGGCCGCCGCCTCGCCTGGGAGTCCCTCCCTGGTTTTTATTCTCAGTACCTCAGGCTC | |
| | CCCTGTGTACTTGGAGGGGCAGGGAGCCCTTTCCTCGGTTCTGGCCTCCAGACCAGGG | |
| | TAAGGGCAGGCCCCTCCAACAGGTGCTCACAGCCACCGAGGCAGGGGCTGCAGCCACC | |
| | CACTGGGAGTCTTGTTTTTATTTATAATAAAATTGTTGGGGACACCTCAAAAAAAAAA | |
| | AAGCCCACAAATTCCCCGAGGGACAATTACGGTACAGCGTCATGACAAAGGCCCACAG | |
| | AGTCAAAACCCAAACCGCGCCCTTACAAGCCAGACGGCACAGTCTCGGTCACGAGACA | |
| | CTCTGGATCATCCACACAAGTACCCAGACAAACGAGCGCACCATCGCTCTACCCCCCC | |
| | ACCGGGCCCGCGGCCCCACCCACCCACAGGACGCACCCGCGCAGCGCCAGCACCCACA | |
| | CCACGCGGCCACCAACGACGACACGCACCACCGACAAGCGACACGCACAGCACCGCCA | |
| | CGAACAAGACGCTGGAGCTCCTCCGGCACGCGCCCGCAGGCCCCCCGCCCACACGACC | |
| | GCCTCCAACACCCCCGCGCGCGCCCTCCACACGAGGACAACGAAACACAAACCAGGCG | |
| | GCACCCCGACGCGCAGCCACCCAGCGCCACCGCACGCACACCCCCCAGCGAGCGACGC | |
| | CCCAACGCGCGCAGCACGGCCACAGGAGCGCGCACCCCCACCGAACCTAGTTGGTAGA | |
| | TAGCAGGTTGC | |
| | ORF Start: ATG at 471 | ORF Stop: TGA at 2412 |
| | SEQ ID NO:188 | 647 aa MW at 68097.9 kD |
| NOV44a, | MAPPLLLLLLASGAAACPLPCVCQNLSESLSTLCAHRGLLFVPPNVDRRTVELRLADN | |
| CG59869-01 Protein | FIQALGPPDFRNNTGLVDLTLSRNAITRIGARAFGDLESLRSLHLDGNRLVELGTGSL | |
| Sequence | RGPVNLQHLILSGNQLGRIAPGAFDDFLESLEDLDLSYNNLRQVPWAGIGANPALHTL | |
| | NLDHNLIDALPPGAFAQLGQLSRLDLTSNRLATLAPDPLFSRGRDAEASPAPLVLSFS | |
| | GNPLHCNCELLWLRRLARPDDLETCASPPGLAGRYFWAVPEGEFSCEPPLIARHTQRL | |
| | WVLEGQRATLRCRALGDPAPTMHWVGPDDRLVGNSSRARAFPNGTLEIGVTGAGDAGG | |
| | YTCIATNPAGEATARVELRVLALPHGGNSSAEGGRPGPSDIAASARTAAEGEGTLESE | |
| | PAVQVTEVTATSGLVSWGPGRPADPVWMFQIQYNSSEDETLIYRIVPASSHHFLLKHL | |
| | VPGADYDLCLLALSPAAGPSDLTATRLLGCAHFSTLPASPLCHALQAHVLGGTLTVAV | |
| | GGVLVAALLVFTVALLVRGRGAGNGRLPLKLSHVQSQTNGGPSPTPKAHPPREPPAPA | |
| | AAQLLSCPGRCRVLRLCQAPGRSLGPTEPLCAWGAARGRVPGGRRQRRAAGRECGVMD | |
| | GQLPVCSKG | |
| | SEQ ID NO:189 | 2451 bp |
| NOV44b, | AGAGTCCTCAGCAGGGTAGCCGAGGCCAGGCCACTTCTGCTGAGGATCGGGCAGGCTG | |
| CG59869-02 DNA | GGGTGTGGGTGTGGCCTGGGGTGGCTCAGGGCTGGAACTGCTGCCTGATTCCTGTGTG | |
| Sequence | CGGAGAAGCTCAGTGGCCGTTTGCTGCCACTGACAAGGATTTCACATGCAGAAGAGAA | |
| | AAGGCCCCCCTCCACCCCCCGCATTCCCTGCCGAGTGAGAGCCAGTGTTTGCTGCCCT | |
| | TGCTGGGGGCGGGTAGGAAACCCTGACCTTCCTCATGCGGAGTCATGAAGCAGAGTCC | |
| | TCGGGAAGGCATCTCCCCAACCTTCCCTCATCTCTGGCGCCCCTCTTGGGCCTCTGAC | |
| | CCAGCCCCTCCCCGGGCCAGGCTCACAGAAGCTGGCTTCTGGGACTGTCCTGGGCCCA | |
| | AGTCGGCACCTGCGCCAGCCCCACCTGTGCCTGGGCTGTGGCCCCTTCCTACAGGGCG | |
| | CTCACCATGGCCCCGCCGCTCCTCCTGCTGCTGCTGGCCAGTGGAGCGGCCGCCTGCC | |
| | CGCTGCCCTGCGTCTGCCAGAACCTGTCCGAGTCGCTCAGCACCCTCTGTGCCCACCG | |
| | AGGCCTGCTGTTTGTGCCGCCCAACGTGGACCGGCGCACAGTGGAGCTCCGGCTGGCT | |
| | GACAACTTCATCCAGGCCCTGGGGCCCCCTGACTTCCGCAACATGACGGGACTGGTGG | |
| | ACCTGACACTGTCTCGCAATGCCATCACCCGCATTGGGGCCCGCGCCTTTGGGGACCT | |
| | CGAGAGCCTGCGTTCCCTCCACCTTGACGGCAACAGGCTGGTGGAGCTGGGCACCGGG | |
| | AGCCTCCGGGGCCCCGTCAATCTGCAGCACCTCATCCTCAGCCGCAACCAGCTGGGCC | |
| | GCATCGCGCCGGGAGCCTTCGACGACTTCCTAGAGAGCCTGGAGGACCTGGACCTGTC | |
| | CTACAACAACCTCCGGCAGGTGCCCTGGGCCGGCATCGGCGCCATGCCTGCCCTGCAC | |
| | ACCCTCAACCTGGACCATAACCTTATTGACGCACTGCCCCCAGGCGCCTTCGCCCAGC | |
| | TCGGTCAGCTCTCCCGCCTGGACCTCACCTCCAACCGCCTGGCCACGCTGGCTCCGGA | |
| | CCCGCTTTTCTCTCGTGGGCGTGATGCAGAGGCCTCTCCCGCCCCCCTGGTGCTGAGC | |
| | TTTAGCGGGAACCCCCTGCACTCCAACTGTGAGCTGCTGTGGCTGCGGCGGCTGGCGC | |
| | GGCCGGACGACCTGGAAACGTGCGCCTCCCCGCCCGGCCTGGCCGCCCGCTACTTCTG | |
| | GCCAGTCCCCGAGGGCGAGTTCTCCTGTGAGCCGCCCCTCATTGCCCGCCACACGCAG | |
| | CGCCTCTGGGTGCTGGAAGGCCAGCGGGCCACGCTGCGGTGCCGGGCCCTGGGTGACC | |
| | CCGCGCCTACCATGCACTGGGTCGGTCCTGACGACCGGTTGGTTGGCAACTCCTCCCG | |
| | AGCCCGGGCTTTCCCCAACGGGACCTTAGAGATTGGGGTGACCGGCGCTGGGGACGCT | |
| | GGGGGCTACACCTGCATCGCCACCAACCCTGCTGGTGAGGCCACAGCCCGAGTAGAAC | |
| | TGCGGGTGCTGGCCTTGCCCCATGGTGGGAACAGCAGTGCCGAGGGGGGCCGCCCCGG | |
| | GCCCTCCGACATCGCCGCCTCCGCTCGCACTGCTGCCGAGGGTGAGGGGACGCTGGAG | |
| | TCTGACCCAGCCGTGCAGGTGACGGAGGTGACCGCCACCTCAGGGCTGGTGAGCTGGG | |
| | GTCCCGGGCGGCCAGCCGACCCAGTGTGGATGTTCCAAATCCAGTACAACAGCAGCGA | |
| | AGATGAGACCCTCATCTACCGGATTGTCCCAGCCTCCAGCCACCACTTCCTGCTGAAG | |
| | CACCCCGTCCCCGGCGCTGACTATGACCTCTGCCTGCTGGCCTTGTCACCGGCCGCTG | |
| | GGCCCTCTGACCTCACGGCCACCAGGCTGCTGGGCTGTGCCCATTTCTCCACGCTGCC | |
| | GGCCTCGCCCCTGTGCCACGCCCTGCAGGCCCACGTGCTGGGCGGGACCCTGACCGTG | |
| | GCCGTGGGGCGTGTGCTGGTGGCTGCAGCCCCTGCCTCGGTGGCTGTGAGCACCTGTT | |
| | GGAGGGGCCTGCCCTTACCCTGGTCTGGAGGCCAGAACCGAGGAAAGGGCTCCCTGCC | |
| | CCTCCAAGTACACAGGGGAGCCTGAGGTACTGAGAATAAAAACCAGGCAGGGACTCCC | |
| | AGGCCAGGCGGCGGCCCCGGGCCCTCTGCCCCACGAGGCTCATCCCTTGGAGCACACA | |
| | GGAAGCTGCCCGTCCATCACACCACACTCTCTTCCAGCCGCTCGGCGCTGCCTCCTAC | |
| | CCCCCGGCACCCTGCCCCGAGCAGCCCCCCATGCACAGAGTGGCTCCGTCGGGCCTAC | |
| | GCTCCTCCCAGGCGCCTGGCATAACCGTAGCACCCGGCATCTCCCAGGTCCAGAGACC | |
| | AGCTGCGCTGCGGCC | |
| | ORF Start: ATG at 471 | ORF Stop: TGA at 2169 |
| | SEQ ID NO:190 | 566 aa MW at 59683.1 kD |
| NOV44b, | MAPPLLLLLLASGAAACPLPCVCQNLSESLSTLCAHRGLLFVPPNVDRRTVELRLADN | |
| CG59869-02 Protein | FIQALGPPDFRNMTGLVDLTLSRNAITRIGARAFGDLESLRSLHLDGNRLVELGTGSL | |
| Sequence | RGPVNLQHLILSGNQLGRIAPGAFDDFLESLEDLDLSYNNLRQVPWAGIGAMPALHTL | |
| | NLDHNLIDALPPGAFAQLGQLSRLDLTSNRLATLAPDPLFSRGRDAEASPAPLVLSFS | |
| | GNPLHCNCELLWLRRLARPDDLETCASPPGLAGRYFWAVPEGEFSCEPPLIARHTQRL | |
| | WVLEGQPATLRCRALGDPAPTMHWVGPDDRLVGNSSRARAFPNGTLEIGVTGAGDAGG | |
| | YTCIATNPAGEATARVELRVLALPHGGNSSAEGGRPGPSDIAASARTAAEGEGTLESE | |
| | PAVQVTEVTATSGLVSWGPGRPADPVWMFQIQYNSSEDETLIYRIVPASSHHFLLKHP | |
| | VPGADYDLCLLALSPAAGPSDLTATRLLGCAHFSTLPASPLCHALQAHVLGGTLTVAV | |
| | GGVLVAAAPASVAVSTCWRGLPLPWSGGQNRGKGSLPLQVHRGA | |
| | SEQ ID NO:191 | 2563 bp |
| NOV44c, | TCCCAATCTGGAGGGGAACGTTGCACCCCAGCCCCAGGAGGCCCTGCCCGTGTGAAGA | |
| CG59869-03 DNA | GCCAGCCAAGTGGGCACCTGCGCCAGCCCCACCTGTGCCTGCCTGTGGCCCCTTCCTA | |
| Sequence | CAGGGCGCTCACCATGCCCCCCGCGCTCCTGCTGCTGCTGCTGGCCAGTGGAGCGGCC | |
| | GCCTGCCCCCTGCCCTGCGTCTGCCAGAACCTGTCCGAGTCGCTCAGCACCCTCTGTG | |
| | CCCACCGACGCCTGCTGTTTCTGCCGCCCAACGTGGACCGGCGCACAGTGGAGCTGCG | |
| | GCTGGCTGACAACTTCATCCAGGCCCTCGGGCCCCCCGACTTCCGCAACATGACGGGA | |
| | CTGGTGGACCTGACACTGTCTCGCAATGCCATCACCCGCATTGCGGCCCGCGCCTTTG | |
| | GGGACCTCGAGAGCCTACGTTCCCTCCACCTTGACGGCAACAGGCTGGTGGAGCTGGG | |
| | CACCGCGAGCCTCCGGGGCCCCGTCAATCTGCAGCACCTCATCCTCAGCGGCAACCAG | |
| | CTGGGCCGCATCGCGCCGGGAGCCTTCGACGACTTCCTAGAGAGCCTGGAGGACCTGG | |
| | ACCTGTCCTACAACAACCTCCGGCAGGTGCCCTGGGCCGGCATCGGCGCCATGCCTGC | |
| | CCTGCACACCCTCAACCTGGACCATAACCTTATTGACGCACTGCCCCCAGGCGCCTTC | |
| | GCCCAGCTCGGTCAGCTCTCCCGCCTGGACCTCACCTCCAACCGCCTGGCCACGCTGG | |
| | CTCCGGACCCGCTTTTCTCTCGTGGGCGTGATGCAGAGGCCTCTCCCGCCCCCCTGGT | |
| | GCTGAGCTTTAGCGGGAACCCCCTGCACTGCAACTGTGAGCTGCTCTGGCTGCGGCGG | |
| | CTGGCGCGGCCGGACGACCTGGAAACGTGCGCCTCCCCGCCCGGCCTGGCCGGCCGCT | |
| | ACTTCTGGGCAGTGCCCGAGGGCGAGTTCTCCTGTGAGCCGCCCCTCATTGCCCGCCA | |
| | CACGCAGCGCCTCTGGGTGCTGGAAGGCCAGCGGGCCACGCTGCGGTGCCGGGCCCTG | |
| | GGTGACCCCGCGCCTACCATGCACTGGGTCGGTCCTGACGACCGGTTGGTTGGCAACT | |
| | CCTCCCGAGCCCGGGCTTTCCCCAACGGGACCTTAGAGATTGGGGTGACCGGCGCTGG | |
| | GGACGCTGGGGGCTACACCTGCATCGCCACCAACCCTGCTGGTGAGGCCACAGCCCGA | |
| | GTAGAACTGCGGGTGCTGGCCTTGCCCCATGGTGGGAACAGCAGTCCCGAGGGGGGCC | |
| | GCCCGGGCCCTCGGACATCGGCCCCATGGTGGGAACAGCAGTGCCGAGGGGGGCCGCC | |
| | CGGGCCCTCGGACATCGCCGCCTCCGCTCGCACTGCTGCCGAGGGTGAGGGGACGCTG | |
| | GAGTCTGAGCCAGCCGTGCAGGTGACGGAGGTGACCGCCACCTCAGGGCTGGTGAACT | |
| | GGGGTCCCCGGCAGCCAGCGACCCACGTGTGGATGTTCCAAATCCAGTACAACAGCAG | |
| | CGAAGATGAGACCCTCATCTGCCGGATTGTCCCAGCCTCCAGCCACCACTTCCTGCTG | |
| | AAGCACCTCGTCCCCGGCGCTGACTATGACCTCTGCCTGCTCGCCTTGTCACCGGCCG | |
| | CTGGGCCCTCTGACCTCACGGCCACCAGGCTGCTGGGCTGTGCCCATTTCTCCACGCT | |
| | GCCGGCCTCGCCCCTGTGCCACGCCCTGCAGGCCCACGTGCTGGGCGGGACCCTGACC | |
| | GTGGCCGTGGGGGGTGTGCTGGTGGCTGCCTTACTGGTCTTCACTGTGGCCTTGCTCG | |
| | TTCGGGGCCGGGGGGCCGGAAATGGCCGCCTCCCCCTCAAGCTCAGCCACGTCCAGTC | |
| | CCACACCAATGGAGGCCCCAGCCCCACACCCAAGGCCCACCCGCCGCGGAGCCCCCCG | |
| | CCCCGGCCGCAGCGCAGCTGCTCTCTGGACCTGGGAGATGCCGGGTGCTACGGTTATG | |
| | CCAGGCGCCTGGGAGGAGCTTGGGCCCGACGGAGCCACTCTGTCCATGGGGGGCTGCT | |
| | CGGGGCAGGGTGCCGGGGGGTAGGAAGGGAGCCCTCCTGGAGAAGGCGCGAGTCTTGC | |
| | TGTGCTGAGGAGCCTGCCGTGGACCGCCTCAGCGCCCCCTACACCACTCTCGCCCTGA | |
| | GGACCAGCACCCTGAGGAAGCTCCAGGGAGGCAGGTATCAGCTCGGCAGACACAAGAG | |
| | CTTGCATGGCCAGGGCCCCCACAGTGAAAATGACCCCGAGTTGGGGCAAGCTCCCCAT | |
| | CAAGGGAGACCGCCGCGGAGCCCCCCGCCCCGGCCGCAGCGCAGCTGCTCTCTGGACC | |
| | TGGGAGATGCCGGGTGCTACGGTTATGCCAGGCGCCTGGGAGGAGCTTGGGCCCGACG | |
| | GAGCCACTCTGTGCATGGGGGGCTGCTCGGGGCAGGGTGCCGGGGGGTAGGAGGCAGC | |
| | GCCGAGCGGCTGGAAGAGAGTGTGGTGTGATGGACGGGCAGCTTCCTGTGTGCTCCAA | |
| | GGGATGAGCCTCGTGCGGCAGAGGGCCCGGGGCCGCCGCCTGGCCTGGGAGTCCCTCC | |
| | CTGGTTTTTAT | |
| | ORF Start: ATG at 130 | ORF Stop: TGA at 2464 |
| | SEQ ID NO:192 | 778 aa MW at 82472.5 kD |

| | |
|---|---|
| NOV44c, | MAPALLLLLLASGAAACPLPCVCQNLSESLSTLCAHRGLLFVPPNVDRRTVELRLADN |
| CG59869-03 Protein | FIQALGPPDFRNMTGLVDLTLSRNAITRIGARAFGDLESLRSLHLDGNRLVELGTGSL |
| Sequence | RGPVNLQHLILSGNQLGRIAPGAFDDFLESLEDLDLSYNNLRQVPWAGIGAMPALHTL |
| | NLDHNLIDALPPGAFAQLGQLSRLDLTSNRLATLAPDPLFSRGRDAEASPAPLVLSFS |
| | GNPLHCNCELLWLRRLARPDDLETCASPPGLAGRYFWAVPEGEFSCEPPLIARHTQRL |
| | WVLEGQRATLRCRALGDPAPTMHWVGPDDRLVGNSSRARAFPNGTLEIGVTGAGDAGG |
| | YTCIATNPAGEATARVELRVLALPHGGNSSAEGGRPGPRTSAPWWEQQCRGGPPGPSD |
| | IAASARTAAEGEGTLESEPAVQVTEVTATSGLVNWGPRQPATHVWMFQIQYNSSEDET |
| | LICRIVPASSHHFLLKHLVPGADYDLCLLALSPAAGPSDLTATRLLGCAHFSTLPASP |
| | LCHALQAHVLGGTLTVAVGGVLVAALLVFTVALLVRGRGAGNGRLPLKLSHVQSQTNG |
| | GPSPTPKAHPPRSPPPRPQRSCSLDLGDAGCYGYARRLGGAWARRSHSVHCGLLGAGC |
| | RGVGREPSWRRRESCCAEEPAVDRLSAPYTTLALRTSTLRKLQGGRYQLGRHKSLHGQ |
| | GPHSENDPELGQAPHQGRPPRSPPPRPQRSCSLDLGDAGCYGYARRLGGAWARRSHSV |
| | HGGLLGAGCRGVGGSAERLEESVV |

Sequence comparison of the above protein sequences yields the following sequence relationships shown in Table 44B.

TABLE 44B
Comparison of NOV44a against NOV44b through NOV44c.

| | NOV44a Residues/ | Identities/Similarities |
|---|---|---|
| Protein Sequence | Match Residues | for the Matched Region |
| NOV44b | 21 . . . 512 | 477/492 (96%) |
| | 21 . . . 512 | 477/492 (96%) |
| NOV44c | 21 . . . 563 | 496/561 (88%) |
| | 21 . . . 581 | 498/561 (88%) |

Further analysis of the NOV44a protein yielded the following properties shown in Table 44C.

TABLE 44C
Protein Sequence Properties NOV44a

| | |
|---|---|
| PSort | 0.4600 probability located in plasma membrane; 0.1000 |
| analysis: | probability located in endoplasmic reticulum (membrane); |
| | 0.1000 probability located in endoplasmic reticulum (lumen); |
| | 0.1000 probability located in outside |
| SignalP | Cleavage site between residues 17 and 18 |
| analysis: | |

A search of the NOV44a protein against the Geneseq database, a proprietary database that contains sequences published in patents and patent publication, yielded several homologous proteins shown in Table 44D.

TABLE 44D
Geneseq Results for NOV44a

| | | NOV44a | Identities/ | |
|---|---|---|---|---|
| Geneseq | Protein/Organism/Length [Patent #, | Residues/ | Similarities for | Expect |
| Identifier | Date] | Residues | Region | Value |
| AAB70072 | Human secreted protein #11 - | 168 . . . 577 | 408/410 (99%) | 0.0 |
| | Homo sapiens, 468 aa. [WO200112776-A2, | 1 . . . 410 | 408/410 (99%) | |
| | 22-FEB-2001] | | | |
| AAB12448 | Human hh00149 protein SEQ ID NO: 4 - | 9 . . . 577 | 330/590 (55%) | e−180 |
| | Homo sapiens, 785 aa. | 4 . . . 592 | 408/590 (68%) | |
| | [WO200031255-A1, 02-JUN-2000] | | | |
| AAB09968 | Human brain-specific transmembrane | 9 . . . 577 | 330/590 (55%) | e−180 |
| | glycoprotein - Homo sapiens, 789 aa. | 8 . . . 596 | 408/590 (68%) | |
| | [WO200031256-A1, 02-JUN-2000] | | | |
| AAU28092 | Novel human secretory protein, Seq ID | 9 . . . 577 | 329/590 (55%) | e−179 |
| | No 261 - Homo sapiens, 789 aa. | 8 . . . 596 | 407/590 (68%) | |
| | [WO200166689-A2, 13-SEP-2001] | | | |
| AAM39059 | Human polypeptide SEQ ID NO 2204 - | 9 . . . 577 | 329/590 (55%) | e−179 |
| | Homo sapiens, 789 aa. | 8 . . . 596 | 407/590 (68%) | |
| | [WO200153312-a1, 26-JUL-2001] | | | |

In a BLAST search of public sequence databases, the NOV44a protein was found to have homology to the proteins shown in the BLASTP data in Table 44E.

TABLE 44E
Public BLASTP Results for NOV44a

| | | NOV44a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9ULH4 | KIAA1246 PROTEIN - Homo sapiens | 9 . . . 577 | 330/590 (55%) | e−179 |
| | (Human), 832 aa (fragment). | 51 . . . 639 | 408/590 (68%) | |
| Q9BE71 | HYPHOTHETICAL 84.7 KDA | 9 . . . 577 | 328/590 (55%) | e−178 |
| | PROTEIN - Macaca fascicularis (Crab | 8 . . . 596 | 406/590 (68%) | |
| | eating macaque) (Cynomolgus | | | |
| | monkey), 789 aa. | | | |
| Q9CYK3 | 5730420O05RIK PROTEIN - | 9 . . . 577 | 327/595 (54%) | e−174 |
| | Mus musculus (Mouse), 788 aa. | 8 . . . 601 | 407/595 (67%) | |
| CAD10239 | SEQUENCE 1 FROM PATENT | 4 . . . 582 | 329/608 (54%) | e−171 |
| | WO0172827 - Homo sapiens (Human), | 5 . . . 608 | 398/608 (65%) | |
| | 628 aa. | | | |
| Q9BTN0 | HYPOTHETICAL 66.3 KDA | 4 . . . 582 | 329/608 (54%) | e−171 |
| | PROTEIN - Homo sapiens (Human), | 5 . . . 608 | 398/608 (65%) | |
| | 628 aa. | | | |

PFam analysis indicates that the NOV44a protein contains the domains shown in the Table 44F.

TABLE 44F
Domain Analysis of NOV44a

| | | Identities/ | |
|---|---|---|---|
| | | Similarities | |
| Pfam Domain | NOV44a Match Region | for the Matched Region | Expect Value |
| LRRNT: domain 1 of 1 | 16 . . . 47 | 12/33 (36%) | 1 |
| | | 20/33 (61%) | |
| LRR: domain 1 of 7 | 49 . . . 72 | 8/25 (32%) | 75 |
| | | 15/25 (60%) | |
| LRR: domain 2 of 7 | 73 . . . 96 | 8/25 (32%) | 1.4 |
| | | 15/25 (60%) | |
| LRR: domain 3 of 7 | 97 . . . 120 | 7/25 (28%) | 0.82 |
| | | 19/25 (76%) | |
| LRR: domain 4 of 7 | 121 . . . 144 | 9/25 (36%) | 0.022 |
| | | 17/25 (68%) | |
| LRR: domain 5 of 7 | 146 . . . 169 | 12/25 (48%) | 0.049 |
| | | 19/25 (76%) | |
| LRR: domain 6 of 7 | 170 . . . 193 | 9/25 (36%) | 0.0096 |
| | | 18/25 (72%) | |
| LRR: domain 7 of 7 | 194 . . . 214 | 9/25 (36%) | 86 |
| | | 15/25 (60%) | |
| LRRCT: domain 1 of 1 | 234 . . . 279 | 21/54 (39%) | 1.1e−05 |
| | | 37/54 (69%) | |
| ig: domain 1 of 1 | 295 . . . 353 | 15/62 (24%) | 9e−10 |
| | | 43/62 (69%) | |
| fn3: domain 1 of 1 | 404 . . . 487 | 21/86 (24%) | 2.7e−08 |
| | | 58/86 (67%) | |

### Example 45

The NOV45 clone was analyzed, and the nucleotide and polypeptide sequences are shown in Table 45A.

TABLE 45A
NOV45 Sequence Analysis

| | SEQ ID NO:193 | 704 bp |
|---|---|---|
| NOV45a, | GAAGCGCQGAGGCCCGTCCCGGTGGCCGGGGAGCGGGCGGGTGGGGGCGCCATGTGGT | |
| CG59859-01 DNA | TCATGTACCTGCTGAGCTGGCTGTCGCTCTTCATCCAGGTGGCCTTCATCACGCTGGC | |
| Sequence | TGTCGCGGCTGGACTCTATTACCTGGCAGAACTGATAGAAGAATACACAGTGGCCACC | |
| | AGCAGGATCATAAAATACATGATCTGGTTCTCCACCGCTGTACTGATTGGCCTCTACG | |
| | TCTTTGAGCGCTTCCCCACCAGCATGATTGGAGTGGGCCTATTCACCAACCTCGTCTA | |
| | CTTTGGCCTCCTCCAGACCTTCCCCTTCATCATGCTCACCTCGCCTAACTTCATCCTG | |
| | TCGTGTGGACTAGTGGTGGTGAATCATTACCTAGCATTTCAGTTTTTTGCAGAAGAAT | |
| | ATTATCCCTTCTCAGAGGTCCTGGCCTATTTCACTTTCTGCCTGTGGATAATTCCGTT | |
| | TGCGTTTTTTGTGTCACTTTCGGCCGGGGAGAACGTCCTGCCCTCTACCATGCAGCCA | |
| | GGAGATGATGTCGTCTCCAATTATTTCACCAAAGGCAAGCGGGGCAAACGCTTAGGGA | |
| | TCCTGGTTGTCTTCTCCTTCATCAAAGAGGCCATTCTACCCAGTCGTCAGAAGATATA | |
| | CTGACCCCCATGCAGGCAGGATGTGGGGGGCAAGATCAGGAGAGTCAGGCCCCTGGGC | |
| | CTCTATGC | |
| | ORF Start: ATG at 52 | ORF Stop: TGA at 640 |
| | SEQ ID NO:194 | 196 aa MW at 22537.4 kD |
| NOV45a, | MWFMYLLSWLSLFIQVAFITLAVAAGLYYLAELIEEYTVATSRIIKYMIWFSTAVLIG | |
| CG59859-01 Protein | LYVFERFPTSMIGVGLFTNLVYFGLLQTFPFIMLTSPNFILSCGLVVVNHYLAFQFFA | |
| Sequence | EEYYPFSEVLAYFTFCLWIIPFAFFVSLSAGENVLPSTMQPGDDVVSNYFTKGKRGKR | |
| | LGILVVFSFIKEAILPSRQKIY | |
| | SEQ ID NO:195 | 619 bp |
| NOV45b, | CATGTGGTTCATGTACCTGCTGAGCTGGCTGTCGCTCTTCATCCAGGTGGCCTTCATC | |
| CG59859-02 DNA | ACGCTGGCTGTCGCGGCTGGACTCTATTACCTGGCAGAACTGATAGAAGAATACACAG | |
| Sequence | TGGCCACCAGCAGGATCATAAAATACATGATCTGGTTCTCCACCGCTGTACTGATTGG | |
| | CCTCTACGTCTTTGAGCGCTTCCCCACCAGCATGATTGGAGTGGGCCTATTCACCAAC | |
| | CTCGTCTACTTTGGCCTCCTCCAGACCTTCCCCTTCATCATGCTGACCTCGCCTAACT | |
| | TCATCCTGTCGTGTGGACTAGTGGTGGTGAATCATTACCTAGCATTTCAGTTTTTTGC | |
| | AGAAGAATATTATCCCTTCTCAGAGGTCCTGGCCTATTTCACTTTCTGCCTGTGGATA | |
| | ATTCCGTTTGCGTTTTTTGTGTCACTTTCGGCCGGGGAGAACGTCCTGCCCTCTACCA | |
| | TGCAGCCAGGAGATGATGTCGTCTCCAATTATTTCACCAAAGGCAAGCGGGGCAAACG | |
| | CTTAGGGATCCTGGTTGTCTTCTCCTTCATCAAAGAGGCCATTCTACCCAGTCGTCAG | |
| | AAGATATACTGACCCCCATGCAGGTACATGAACCACATG | |
| | ORF Start: ATG at 2 | ORF Stop: TGA at 590 |
| | SEQ ID NO:196 | 196 aa MW at 22537.4 kD |

| | |
|---|---|
| NOV45b, | MWFMYLLSWLSLFIQVAFITLAVAAGLYYLAELIEEYTVATSRIIKYMIWFSTAVLIG |
| CG59859-02 Protein | LYVFERFPTSMIGVGLFTNLVYFGLLQTFPFIMLTSPNFILSCGLVVVNHYLAFQFFA |
| Sequence | EEYYPFSEVLAYFTFCLWIIPFAFFVSLSAGENVLPSTMQPGDDVVSNYFTKGKRGKR |
| | LGILVVFSFIKEAILPSRQKIY |

Sequence comparison of the above protein sequences yields the following sequence relationships shown in Table 45B.

TABLE 45B
Comparison of NOV45a against NOV45b and NOV45c.

| | NOV45a Residues/ | Identities/Similarities |
|---|---|---|
| Protein Sequence | Match Residues | for the Matched Region |
| NOV45b | 1 . . . 196 | 196/196 (100%) |
| | 1 . . . 196 | 196/196 (100%) |

Further analysis of the NOV45a protein yielded the following properties shown in Table 45C.

TABLE 45C
Protein Sequence Properties NOV45a

| | |
|---|---|
| PSort | 0.6400 probability located in plasma membrane; 0.4600 |
| analysis: | probability located in Golgi body; 0.3700 probability located |
| | in endoplasmic reticulum (membrane); 0.1000 probability |
| | located in endoplasmic reticulum (lumen) |
| SignalP | Cleavage site between residues 25 and 26 |
| analysis: | |

A search of the NOV45a protein against the Geneseq database, a proprietary database that contains sequences published in patents and patent publication, yielded several homologous proteins shown in Table 45D.

TABLE 45D
Geneseq Results for NOV45a

| | | NOV45a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAU12255 | Human PRO4325 polypeptide | 1 . . . 196 | 196/196 (100%) | e−111 |
| | sequence - Homo sapiens, 196 aa. | 1 . . . 196 | 196/196 (100%) | |
| | [WO200140466-A2, 07-JUN-2001] | | | |
| AAM40045 | Human polypeptide SEQ ID NO 3190 - | 1 . . . 196 | 196/196 (100%) | e−111 |
| | Homo sapiens, 196 aa. | 1 . . . 196 | 196/196 (100%) | |
| | [WO200153312-A1, 26-JUL-2001] | | | |
| AAM41831 | Human polypeptide SEQ ID NO 6762 - | 1 . . . 173 | 173/173 (100%) | 5e−98 |
| | Homo sapiens, 203 aa. | 31 . . . 203 | 173/173 (100%) | |
| | [WO200153312-A1, 26-JUL-2001] | | | |
| AAM93368 | Human polypeptide, SEQ ID NO: 2935 - | 91 . . . 196 | 106/106 (100%) | 6e−57 |
| | Homo sapiens, 106 aa. [EP1130094- | 1 . . . 106 | 106/106 (100%) | |
| | A2, 05-SEP-2001] | | | |
| ABB18676 | Protein #675 encoded by probe for | 51 . . . 101 | 51/51 (100%) | 8e−23 |
| | measuring heart cell gene expression - | 1 . . . 51 | 51/51 (100%) | |
| | Homo sapiens, 51 aa. [WO200157274- | | | |
| | A2, 09-AUG-2001] | | | |

In a BLAST search of public sequence databases, the NOV45a protein was found to have homology to the proteins shown in the BLASTP data in Table 45E.

TABLE 45E
Public BLASTP Results for NOV45a

| Protein | | NOV45a | Identities/ | |
|---|---|---|---|---|
| Accession | | Residues/ | Similarities for | Expect |
| Number | Protein/Organism/Length | Residues | Portion | Value |
| Q62302 | TESTIS EXPRESSED PROTEIN 261 | 1 . . . 196 | 195/196 (99%) | e−110 |
| | (TEG-261) (TESTIS EXPRESSED | 1 . . . 196 | 196/196 (99%) | |
| | GENE 261) - Mus musculus (Mouse), | | | |
| | 196 aa. | | | |
| AAH20251 | HYPOTHETICAL 17.0 KDA | 48 . . . 196 | 149/149 (100%) | 1e−82 |
| | PROTEIN - Homo sapiens (Human), | 1 . . . 149 | 149/149 (100%) | |
| | 149 aa. | | | |
| Q96FM0 | UNKNOWN (PROTEIN FOR | 84 . . . 196 | 113/113 (100%) | 1e−60 |
| | IMAGE: 3855224) - Homo sapiens | 1 . . . 113 | 113/113 (100%) | |
| | (Human), 113 aa (fragment). | | | |
| Q9W1R8 | CG3500 PROTEIN - Drosophila | 1 . . . 194 | 99/198 (50%) | 9e−49 |
| | melanogaster (Fruit fly), 198 aa. | 1 . . . 195 | 135/198 (68%) | |
| JC5386 | steroidogenic acute regulatory protein - | 48 . . . 133 | 86/86 (100%) | 7e−45 |
| | rat, 362 aa. | 1 . . . 86 | 86/86 (100%) | |

PFam analysis predicts that the NOV45a protein contains the domains shown in the Table 45F.

TABLE 45F
Domain Analysis of NOV45a

| | | | |
|---|---|---|---|
| | | Identities/ | |
| | NOV45a | Similarities | Expect |
| Pfam Domain | Match Region | for the Matched Region | Value |

No Significant Matches Found

### Example 46

The NOV46 clone was analyzed, and the nucleotide and polypeptide sequences are shown in Table 46A.

TABLE 46A
NOV46 Sequence Analysis

| | SEQ ID NO:197 | 4798 bp |
|---|---|---|
| NOV46a, | GAAACAGGTCAAAATGAGCAAGAGACGCATGAGCGTGGGTCAGCAAACATGGGCTCTT | |
| CG59913-01 DNA | CTCTGCAAGAACTGTCTCAAAAAATGGAGAATGAAAAGACAGACCTTGTTGGAATGGC | |
| Sequence | TCTTTTCATTTCTTCTGGTACTGTTTCTGTACCTATTTTTCTCCAATTTACATCAAGT | |
| | TCATGACACTCCTCAAATGTCTTCAATGGATCTGGGACGTGTAGATAGTTTTAATGAT | |
| | ACTAATTATGTTATTGCATTTGCACCTGAATCCAAAACTACCCAAGAGATAATGAACA | |
| | AAGTGGCTTCAGCCCCATTCCTAGCAGGAAGAACAATCATGGGGTGGCCTGATGAAAA | |
| | AAGCATGGATGAATTGGATTTGAACTATTCAATAGACGCACTGAGAGTCATCTTTACT | |
| | GATACCTTCTCCTACCATTTGAAGTTTTCTTGGGGACATAGAATCCCCATGATGAAAG | |
| | AGCACAGAGACCATTCAGCTCACTGTCAAGCAGTGAATGAAAAAATGAAGTGTGAAGG | |
| | TTCAGAGTTCTGGGAGAAAGGCTTTGTACCTTTTCAAGCTGCCATTAATGCTGCTATC | |
| | ATAGAAGTGAGTACAAATCATTCAGTGATGGAACAGCTGATGTCAGTTACTGGTGTAC | |
| | ATATGAAGATATTACCTTTTGTTGCCCAAGGAGGAGTTGCAACTGATTTTTTCATTTT | |
| | CTTTTGCATTATTTCTTTTTCTACATTTATATACTATGTATCAGTCAATGTTACACAA | |
| | GAAAGACAATACATTACGTCATTGATGACAATGATGGGACTCCGAGAGTCAGCATTCT | |
| | GGCTTTCCTGGGGTTTGATGTATGCTGGCTTCATCCTTATCATGGCCACTTTAATGGC | |
| | TCTTATTGTAAAATCTGCACAAATTGTCGTCCTGACTGGTTTTGTGATGGTCTTCACC | |
| | CTCTTTCTCCTCTATGGCCTGTCTTTGGTGAGTTTAGCTTTCCTGATGAGTGTGTTGA | |
| | TAAAGAAACCTTTCCTTACGGGCTTGGTTGTGTTTCTCCTTATTGTCTTTTGGGGGAT | |
| | CCTGGGATTCCCAGCATTGTATACACGTCTTCCTGCATTTTTGGAATGGACTTTGTGT | |
| | CTTCTTAGCCCCTTTGCCTTCACTGTTGGGATGGCCCAGCTTATACATTTGGACTATG | |
| | ATGTGAATTCTAATGCCCACTTGCATTCTTCACAAAATCCATACCTCATAATAGCTAC | |
| | TCTTTTCATGTTGGTTTTTGACACCCTTCTGTATTTGGTATTGACATTATATTTTGAC | |
| | AAAATTTTGCCCGGTGAATATGGACATCGATGTTCTCCCTTGTTTTTCCTGAAATCCT | |
| | GTTTTTGGTTTCAACACGGAAGGGCTAATCATGTGGTCCTTGAGAATGAAACAGATTC | |
| | TGATCCTACACCTAATGACTGTTTTGAACCAGTGTCTCCAGAATTCTGTGGGAAAGAA | |
| | GCCATCAGAATCAAAAATCTTAAAAAAGAATATGCAGGGAAGTGTGAGAGAGTAGAAG | |
| | CTTTGAAAGGTGTGGTGTTTOACATATATGAAGGCCAGATCACTGCCCTCCTTGGTCA | |
| | CAGTGGAGCTGGAAAAACTACCCTGTTAAACATACTTAGTGGGTTGTCAGTTCCAACA | |
| | TCAGGTTCAGTCACTGTCTATAATCACACACTTTCAAGAATGGCTCATATAGAAAATA | |
| | TCAGCAAGTTCACTGGATTTTGTCCACAATCCAATGTGCAATTTGGATTTCTCACTGT | |
| | GAAAGAAAACCTCAGGCTGTTTGCTAAAATAAAAGGGATTTTGCCACATGAAGTGGAG | |
| | AAAGAGGTATTGCTATTGGATGAACCGACTCCTGCATTGCATCCTCTTTCAAGGCACC | |
| | GAATATGGAATCTCCTGAAAGAGGGGAAATCAGACAGAGTAATTCTCTTCAGCACCCA | |
| | GTTTATAGATGAGGCTGACATTCTGGCGGACAGGAAGGTGTTCATATCCAATGGGAAG | |
| | CTGAAGTGTGCAGGCTCTTCTCTGTTCCTTAAGAAGAAATGGGGCATAGGCTACCATT | |
| | TAAGTTTGCATCTGAATGAAAGGTGTGATCCAGAGAGTATAACATCACTGGTTAAGCA | |
| | GCACATCTCTGATGCCAAATTGACAGCACAAAGTGAAGAAAAACTTGTATATATTTTG | |
| | CCTTTGGAAACGACAAACAAATTTCCAGAACTTTACAGGGATCTTGATAGATGTTCTA | |
| | ACCAAGGCATTGAGGATTATGGTGTTTCCATAACAACTTTGAATGAGGTGTTTCTGAA | |
| | ATTAGAAGGAAAATCAACTATTGATGAATCAGATATTGGAATTTGGGGACAATTACAA | |
| | ACTGATGGGGCAAAAGATATAGGAAGCCTTGTTGAGCTGGAACAAGTTTTGTCTTCCT | |
| | TCCACGAAACAAGGAAAACAATCAGTGGCGTGGCGCTCTGGAGGCAGCAGGTCTGTGC | |
| | AATAGCAAAAGTTCGCTTCCTAAAGTTAAAGAAAGAAAGAAAAAGCCTGCTGCATAGA | |
| | TTATTGCTTTTTGGTATTAGCTTTATCCCTCAACTTTTGGAACATCTATTCTACGAGT | |
| | CATATCAGAAAAGTTACCCGTGGGAACTGTCTCCAAATACATACTTCCTCTCACCAGG | |
| | ACAACAACCACAGGATCCTCTGACCCATTTACTGGTCATCAATAAGACAGGTTCAACC | |
| | ATTGATAACTTTTTACATTCACTGAGGCGACAGAACATAGCTATAGAAGTGGATGCCT | |
| | TTGGAACTAGAAATGGCACAGATGACCCATCTTACAATGGTGCTATCATTGTGTCAGG | |
| | TGATGAAAAGGATCACAGATTTTCAATAGCATGTAATACAAAACGGCTGAATTGCTTT | |
| | CCTGTCCTCCTGGATGTCATTAGCAATGGACTACTTGGAATTTTTAATTCGTCAGAAC | |
| | ACATTCAGACTGACAGAAGCACATTTTTTGAAGAGCATATGGATTATGAGTATGGGTA | |
| | CCGAAGTAACACCTTCTTCTGGATACCGATGGCAGCCTCTTTCACTCCATACATTGCA | |
| | ATGAGCAGCATTGGTCACTACAAAGTAAGAGCTCATTCCCAGCTACGGATTTCAGGCC | |
| | TCTACCCTTCTGCATACTGGTTTGCCCAAGCACTGGTGGATGTTTCCCTGTACTTTTT | |
| | GATCCTCCTGCTAATGCAAATAATGGATTATATTTTTAGCCCAGAGGAGATTATATTT | |
| | ATAATTCAAAACCTGTTAATTCAAGTAAGTGGCAGCAATTTTGAAATGGTTTTATTAG | |
| | ACTATTTTTTCATGAATGCAGTAATATCATTAATCTTAAACCAAAGAACTTCAAATTA | |
| | CCTGTGCTATTGCATAGTTTTGGTGGTCATCTTCTCGATAGTTGCTACTGATCTAAAT | |
| | GAATATGGATTTCTAGGGCTATTTTTTGGCACCATGTTAATACCTCCCTTCACATTGA | |
| | TTGGCTCTCTATTCATTTTTTCTGAGGTAAGTAGTTCCACTTATAGCTCAAGAAACAA | |
| | AATTGTCCTTTTACCTTTTATTTGCAAAAGAGTGGGGTACCTTCATTTTCTCATTTTT | |
| | CTTTTCATTCTGCGATGCCTAGAAATGAACTGCAGGAAGAAACTAATGAGAAAGGATC | |
| | CTGTGTTCAGAATTTCTCCAAGAAGCAACGCTATTTTTCCAAACCCAGAAGAGCCTGA | |
| | AGGAGAGGAGGAAGATATCCAGATGGAAAGAATGAGAACAGTGAATGCTATGGCTGTG | |
| | CGAGACTTTGATGAGACACCCGTCATCATTGCCAGCTGTCTACGGAAGGAATATGCAG | |
| | GCAAAAAGAAAAATTGCTTTTCTAAAAGGAAGAAAAAAATTGCCACAAGAAATGTCTC | |
| | TTTTTGTGTTAAAAAAGGTGAAGTTATAGGACTGTTAGGACACAATGGAGCTGGTAAA | |
| | AGTACAACTATTAAGATGATAACTGGAGACACAAAACCAACTGCAGGACAGGTCATTT | |
| | TGAAAGGGAGCGGTGGAGGGGAACCCCTGGGCTTCCTGGGGTACTGCCCTCAGGAGAA | |
| | TGCGCTGTGGCCCAACCTGACAGTGAGGCAGCACCTGGAGGTGTACGCTGCCGTGAAA | |
| | GGTCTCAGGAAAGGGGACGCAATGATCGCCATCACACGGTTAGTGGATGCGCTCAAGC | |
| | TGCAGGACCAGCTGAAGGCTCCCGTGAAGACCTTGTCAGAGGGAATAAAGCGAAAGCT | |
| | GTGCTTTGTGCTGAGCATCCTGGGGAACCCGTCAGTGGTGCTTCTGGATGAGCCGTCG | |
| | ACCGGGATGGACCCCGAGGGGCAGCAGCAAATGTGGCAGGTGATTCGGGCCACCTTTA | |
| | GAAACACGGAGAGGGGCGCCCTCCTGACCACCCACTACATGGCAGAGGCTGAGGCGGT | |
| | GTGTGACCGAGTGGCCATCATGGTGTCAGGAAGCCTGAGGTGTATTGGTTCCATCCAA | |
| | CACCTGAAAAGCAAATTTGGCAAAGACTACCTGCTGGAGATGAAGCTGAAGAACCTGG | |
| | CACAAATGGAGCCCCTCCATGCAGAGATCCTGAGGCTTTTCCCCCAGGCTGCTCAGCA | |
| | GGAAAGGTTCTCCTCCCTGATGGTCTATAAGTTGCCTGTTGAGGATGTGCGACCTTTA | |
| | TCACAGGCTTTCTTCAAATTAGAGATAGTTAAACAGAGTTTCGACCTGGAGGAGTACA | |
| | GCCTCTCACAGTCTACCCTGGAGCAGGTTTTCCTGGAGCTCTCCAAGGAGCAGGAGCT | |
| | GGGTGATCTTGAAGAGGACTTTGATCCCTCGGTGAAGTGGAAACTCCTCCTGCAGGAA | |
| | GAGCCTTAAAGCTCCAAATACCCTATATCTTTCTTTAATCCT | |
| | ORF Start: ATG at 14 | ORF Stop: TAA at 4763 |
| | SEQ ID NO:198 | 1583 aa MW at 179792.1 kD |

| | |
|---|---|
| NOV46a, | MSKRRMSVGQQTWALLCKNCLKKWRMKRQTLLEWLFSFLLVLFLYLFFSNLHQVHDTP |
| CG59913-01 Protein | QMSSMDLGRVDSFNDTNYVIAFAPESKTTQEIMNKVASAPFLAGRTIMGWPDEKSMDE |
| Sequence | LDLNYSIDAVRVIFTDTFSYHLKFSWGHRIPMMKEHRDHSAHCQAVNEKMKCEGSEFW |
| EKGFVAFQAAINAAIIEVSTNHSVMEQLMSVTGVHMKILPFVAQGGVATDFFIFFCII | |
| | SFSTFIYYVSVNVTQERQYITSLMTMMGLRESAFWLSWGLMYAGFILIMATLMALIVK |
| | SAQIVVLTGFVMVFTLFLLYGLSLVSLAFLMSVLIKKPFLTGLVVFLLIVFWGILGFP |
| | ALYTRLPAFLEWTLCLLSPFAFTVGMAQLIHLDYDVNSNAHLDSSQNPYLIIATLFML |
| | VFDTLLYLVLTLYFDKILPGEYGHRCSPLFFLKSCPWFQHGRANHVVLENETDSDPTP |
| | NDCFEPVSPEFCGKEAIRIKNLKKEYAGKCERVEALKGVVFDIYEGQITALLGHSGAG |
| | KTTLLNILSGLSVPTSGSVTVYNHTLSRMADIENISKFTGFCPQSNVQFGFLTVKENL |
| | RLFAKIKGILPHEVEKEVLLLDEPTAGLDPLSRHRIWNLLKEGKSDRVILFSTQFIDE |
| | ADILADRKVFISNGKLKCAGSSLFLKKKWGIGYHLSLHLNERCDPESITSLVKQHISD |
| | AKLTAQSEEKLVYILPLERTNKFPELYRDLDRCSNQGIEDYGVSITTLNEVFLKLEGK |
| | STIDESDIGIWGQLQTDGAKDIGSLVELEQVLSSFHETRKTISGVALWRQQVCAIAKV |
| | RFLKLKKERKSLLHRLLLFGISFIPQLLEHLFYESYQKSYPWELSPNTYFLSPGQQPQ |
| | DPLTHLLVINKTGSTIDNFLHSLRRQNIAIEVDAFGTRNGTDDPSYNGAIIVSGDEKD |
| | HRFSIACNTKRLNCFPVLLDVISNGLLGIFNSSEHIQTDRSTFFEEHMDYEYGYRSNT |
| | FFWIPMAASFTPYIAMSSIGDYKVPAHSQLRISGLYPSAYWFGQALVDVSLYFLILLL |
| | MQIMDYIFSPEEIIFIIQNLLIQVSGSNFEMVLLDYFFMNAVISLILNQRTSNYLCYC |
| | IVLVVIFSIVATDLNEYGFLGLFFGTMLIPPFTLIGSLFIFSEVSSSTYSSRNKIVLL |
| | PFICKRVGYLHFLIFLFILRCLEMNCRKKLMRKDPVFRISPRSNAIFPNPEEPEGEEE |
| | DIQMERMRTVNAMAVRDFDETPVIIASCLRKEYAGKKKNCFSKRKKKIATRNVSFCVK |
| | KGEVIGLLGHNGAGKSTTIKMITGDTKPTAGQVILKGSGGOEPLGFLCYCPQENALWP |
| | NLTVRQHLEVYAAVKGLRKGDAMIAITRLVDALKLQDQLKAPVKTLSEGIKRKLCFVL |
| | SILGNPSVVLLDEPSTGMDPEGQQQMWQVIRATFRNTERGALLTTHYMAEAEAVCDRV |
| | AIMVSGRLRCIGSIQHLKSKFGKDYLLEMKLKNLAQMEPLHAEILRLFPQAAQQERFS |
| | SLMVYKLPVEDVRPLSQAFFKLEIVKQSFDLEEYSLSQSTLEQVFLELSKEQELGDLE |
| | EDFDPSVKWKLLLQEEP |

Further analysis of the NOV46a protein yielded the following properties shown in Table 46B.

TABLE 46B
Protein Sequence Properties NOV46a

| | |
|---|---|
| PSort | 0.8000 probability located in plasma membrane; 0.6281 |
| analysis: | probability located in mitochondrial inner membrane; |
| | 0.4410 probability located in mitochondrial intermembrane |
| | space; 0.4000 probability located in Golgi body |
| SignalP | Cleavage site between residues 54 and 55 |
| analysis: | |

A search of the NOV46a protein against the Geneseq database, a proprietary database that contains sequences published in patents and patent publication, yielded several homologous proteins shown in Table 46C.

TABLE 46C
Geneseq Results for NOV46a

| | | NOV46a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAM93187 | Human polypeptide, SEQ ID NO: 2559 - | 374 . . . 816 | 442/483 (91%) | 0.0 |
| | Homo sapiens, 483 aa. [EP1130094- | 1 . . . 483 | 442/483 (91%) | |
| | A2, 05-SEP-2001] | | | |
| AAB67309 | ABC transport related protein #3 - | 1222 . . . 1583 | 330/362 (91%) | 0.0 |
| | Homo sapiens, 362 aa. | 1 . . . 362 | 348/362 (95%) | |
| | [WO200107658-A1, 01-FEB-2001] | | | |
| AAU18484 | Human endocrine polypeptide SEQ ID | 1232 . . . 1534 | 216/303 (71%) | e−122 |
| | No 439 - Homo sapiens, 310 aa. | 9 . . . 309 | 253/303 (83%) | |
| | [WO200155364-A2, 02-AUG-2001] | | | |
| AAE09582 | Human gene 2 encoded ABC | 1232 . . . 1534 | 216/303 (71%) | e−122 |
| | transporter protein HDPVY34, SEQ ID | 9 . . . 309 | 253/303 (83%) | |
| | NO: 24 - Homo sapiens, 310 aa. | | | |
| | [WO200155208-A1, 02-AUG-2001] | | | |
| AAM42360 | Human polypeptide SEQ ID NO 93 - | 1232 . . . 1534 | 216/303 (71%) | e−122 |
| | Homo sapiens, 310 aa. | 9 . . . 309 | 253/303 (83%) | |
| | [WO200155449-A1, 02-AUG-2001] | | | |

In a BLAST search of public sequence databases, the NOV46a protein was found to have homology to the proteins shown in the BLASTP data in Table 46D.

TABLE 46D
Public BLASTP Results for NOV46a

| | | NOV46a | Identities/ | |
|---|---|---|---|---|
| Protein | | Residues/ | Similarities for | |
| Accession | | Match | the Matched | Expect |
| Number | Protein/Organism/Length | Residues | Portion | Value |
| AAK30024 | ATP-BINDING CASSETTE A9 - | 1 . . . 1583 | 1521/1629 (93%) | 0.0 |
| | Homo sapiens (Human), 1624 aa. | 1 . . . 1624 | 1541/1629 (94%) | |
| O94911 | (Human), 1581 aa. | 1 . . . 1581 | 1313/1588 (82%) | 0.0 |
| AAK30025 | ATP-BINDING CASSETTE A10 - | 91 . . . 1583 | 920/1557 (59%) | 0.0 |
| | Homo sapiens (Human), 1543 aa. | 1 . . . 1543 | 1159/1557 (74%) | |
| AAK30023 | ATP-BINDING CASSETTE A6 - | 1 . . . 1578 | 906/1630 (55%) | 0.0 |
| | Homo sapiens (Human), 1617 aa. | 1 . . . 1611 | 1153/1630 (70%) | |
| Q96MD8 | CDNA FLJ32506 FIS, CLONE | 1 . . . 816 | 809/856 (94%) | 0.0 |
| | SMINT1000042, WEAKLY SIMILAR | 1 . . . 856 | 813/856 (94%) | |
| | TO ATP-BINDING CASSETTE, SUB- | | | |
| | FAMILY A, MEMBER 3 - Homo | | | |
| | sapiens (Human), 856 aa (fragment). | | | |

PFam analysis indicates that the NOV46a protein contains the domains shown in the Table 46E.

TABLE 46E
Domain Analysis of NOV46a

| | | Identities/ | |
|---|---|---|---|
| | | Similarities | |
| Pfam Domain | NOV46a Match Region | for the Matched Region | Expect Value |
| Cyto_ox_2: domain 1 of 1 | 136 . . . 424 | 54/408 (13%) | 7 |
| | | 190/408 (47%) | |
| PRK: domain 1 of 2 | 512 . . . 525 | 8/14 (57%) | 14 |
| | | 11/14 (79%) | |
| ABC_tran: domain 1 of 2 | 510 . . . 652 | 56/199 (28%) | 3.2e−25 |
| | | 120/199 (60%) | |
| biopterin_H: domain 1 of 1 | 719 . . . 732 | 7/16 (44%) | 9.1 |
| | | 10/16 (62%) | |
| PRK: domain 2 of 2 | 1280 . . . 1299 | 10/20 (50%) | 2.1 |
| | | 15/20 (75%) | |
| ABC_tran: domain 2 of 2 | 1278 . . . 1456 | 60/200 (30%) | 1.6e−44 |
| | | 139/200 (70%) | |

### Example 47

The NOV47 clone was analyzed, and the nucleotide and polypeptide sequences are shown in Table 47A.

TABLE 47A
NOV47 Sequence Analysis

| | SEQ ID NO:199 | 4766 bp |
|---|---|---|
| NOV47a, | CGAATAGGCTAAATGAATATGAAACAGAAAAGCGTGTATCAGCAAACCAAAGCACTTC | |
| CG59909-01 DNA | TGTGCAAGAATTTTCTTAAGAAATGGAGGATGAAAAGAGAGAGCTTATTGGAATGGGG | |
| Sequence | CCTCTCAATACTTCTAGGACTGTGTATTGCTCTGTTTTCCAGTTCCATGAGAAATGTC | |
| | CAGTTTCCTGGAATGGCTCCTCAGAATCTGCGAAGGGTAGATAAATTTAATAGCTCTT | |
| | CTTTAATGGTTGTGTATACACCAATATCTAATTTAACCCAGCACATAATGAATAAAAC | |
| | AGCACTTGCTCCTCTTTTGAAAGGTAGGAGTGTCATTGGGGCACCAAATAAAACACAC | |
| | ATGGACGAAATACTTCTGGAAAATTTACCATATGCTATGGGAATCATCTTTAATGAAA | |
| | CTTTCTCTTATAAGTTAATATTTTTCCAGGGATATAACAGTCCACTTTGGAAAGAAGA | |
| | TTTCTCAGGTACAGCTCATTGCTGGGATGGATATGGTGAGTTTTCATGTACATTGACC | |
| | AAATACTGGAATAGAGGATTTGTGGCTTTACAAACAGCTATTAATACTGCCATTATAG | |
| | AAATCACAACCAATCACCCTGTGATGGAGGAGTTGATGTCAGTTACTGCTATAACTAT | |
| | GAAGACATTACCTTTCATAACTAAAAATCTTCTTCACAATGAGATGTTTATTTTATTC | |
| | TTCTTGCTTCATTTCTCCCCACTTGTATATTTTATATCACTCAATGTAACAAAAGAGA | |
| | GAAAAAAGTCTAAGAATTTGATGAAAATGATGGGTCTCCAAGATTCAGCATTCTGGCT | |
| | CTCCTGGGGTCTAATCTATGCTGGCTTCATCTTTATTATTTCCATATTCGTTACAATT | |
| | ATCATAACATTCACCCAAATTATAGTCATGACTGGCTTCATGGTCATATTTATACTCT | |
| | TTTTTTTATATGGCTTATCTTTGGTAGCTTTGGTGTTCCTGATGAGTGTGCTGTTAAA | |
| | GAAAGCTGTCCTCACCAATTTGGTTGTGTTTCTCCTTACCCTCTTTTGGGGATCTCTG | |
| | GGATTCACTGTATTTTATGAACAACTTCCTTCATCTCTGGAGTGGATTTTGAATATTT | |
| | GTAGCCCTTTTGCCTTTACTACTGGAATGATTCAGGTAATCAAACTGGATTATAACTT | |
| | GAATGGTGTAATTTTTCCTGACCCTTCAGGAGACTCATATACAATGATAGCAACTTTT | |
| | TCTATGTTGCTTTTGGATGGTCTCATCTACTTGCTATTGGCATTATACTTTGACAAAA | |
| | TTGTTTCCAACACCAAAGGACTAATGCTAAGGTTATTGAGAAAGAAATCGATGCTGAG | |
| | CATCCCTCTGATGATTATTTTGAACCAGTAGCTCCTGAATTCCAACGAAAAGPAGCCA | |
| | TCAGAATCAGAAATGTTAAGAAGGAATATAAAGGAAAATCTGGAAAAGTGGAAGCATT | |
| | GAAAGGTTTGCTCTTTGACATATATGAAGGTCAAATCACGGCAATCCTGGGTCACAGT | |
| | GGAGCTGGCAAATCTTCACTGCTAAATATTCTTAATGGATTGTCTGTTCCAACAGAAG | |
| | GTTCAGTTACCATCTATAATAAAAATCTCTCTGAAATGCAAGACTTGGAGGAAATCAG | |
| | AAAGATAACTGGCGTCTGTCCTCAATTCAATGTTCAATTTGACATACTCACCGTGAAG | |
| | GAAAACCTCAGCCTGTTTGCTAAAATAAAAGGGATTCATCTAAAGGAAGTGGAACAAG | |
| | AGATTTTGCTTTTAGATGAACCAACTACTGGATTGGATCCCTTTTCCAGAGATCAAGT | |
| | GTGGACCCTCCTGAGAGAGCGTAGAGCAGATCATGTGATCCTTTTCAGTACCCAGTCC | |
| | ATGGATGAGGCTGACATCCTGGCTGATAGAAAAGTGATCATGTCCAATGGGAGACTGA | |
| | AGTGTGCAGGTTCTTCTATGTTTTTGAAAAGAAGGTGGGGTCTTGGATATCACCTAAG | |
| | TTTACATAGGAATGAAATATGTAACCCAGAACAAATAACATCCTTCATTACTCATCAC | |
| | ATCCCCGATGCTAAATTAAAAACAGAAAACAAAGAAAAGCTTGTATATACTTTGCCAC | |
| | TGGAAAGGACAAATACATTTCCAGATCTTTTCAGTGATCTGGATAAGTGTTCTGACCA | |
| | GGGAGTGACAGGTTATGACATTTCCATGTCAACTCTAAATGAAGTCTTTATGAAACTG | |
| | GAAGGACAGTCAACTATCGAACAAGGTAAAGCCATTTGTATAATTCACAGACAAGTGG | |
| | AGATGATAAGAGACTCAGAAAGCCTCAATGAAATGGAGCTGGCTCACTCTTCCTTCTC | |
| | TGAAATGCAGACAGCTGTGAGTGACATGGGCCTCTGGAGAATGCAAGTCTTTGCCATG | |
| | GCACGGCTCCGTTTCTTAAAGTTAAAACGTCAAACTAAAGTGTTATTGACCCTGTTAT | |
| | TGGTATTTGGAATCGCAATATTCCCTTTGATTGTTGAAAATATAATGTATGCTATGTT | |
| | AAATGAAAAGATCGATTGGGAATTTAAAAACGAATTGTATTTTCTCTCTCCTGGACAA | |
| | CTTCCCCAGGAACCCCGTACCAGCCTGTTGATCATCAATAACACAGGTTCAAATATTG | |
| | AAGATTTTATAAAATCACTGAAGCATCAAAATATACTTTTGGAAGTAGATGACTTTGA | |
| | AAACAGAAATGGTACTGATGGCCTCTCATACAATGGAGCTATCATAGTTTGTTTCTGT | |
| | TTTAAGGATTATAGATTTTCAGTTGTGTGTAATACCAAGAGATTGCACTGTTTTCCAA | |
| | TTCTTATGAATATTATCAGCAATGGGCTACTTCAAATGTTTAATCACACACAACATAT | |
| | TCGAATTGAGTCAAGCCCATTTAGCCACATAGCACTCTGGACTGGGTTGCCGGATGGT | |
| | TCCTTTTTCTTATTTTTGGTTCTATGTAGCATTTCTCCTTATATCACCATGGGCAGCA | |
| | TCAGTCATTACAAGGTAAGAGCTAAGTCCCAGCTATGGATTTCAGGCCTCTACACTTC | |
| | TGCTTACTGGTGTGGGCAGGCACTAGTGGACGTCACCTTCTTCATTTTAATTCTCCTT | |
| | TTAATGTATTTAATTTTCTACATAGAAAACATGCAGTACCTTCTTATTACATATTTCT | |
| | TTTATGCTCACGTTATAGTTACTCCTGGTTATGCAGCTTCTCTTGTCTTCTTCATATA | |
| | TATGATATCATTTATTTTTCGCAAAAGGAGAAAAAACAGTGGCCTTTGGTCATTTTAC | |
| | TTCTTTTTTGCCTCCACCATCATGTTTTCCATCACTTTAATCAATCATTTTGACCTAA | |
| | GTATATTGATTACCACCATGGTATTGGTTCCTTCATATACCTTGCTTGGATTTAAAAC | |
| | TTTTTTGGAAGTGAGAGACCAGGAGCACTACAGAGAATTGAGTGCCACTGATTTTCTA | |
| | GTCTGCTTCATACCCTACTTTCAGACTTTGCTATTCGTTTTTGTTCTAAGATGCATGG | |
| | AACTAAAATGTGGAAAGAAAAGAATGCGAAAAGATCCTGTTTTCAGGATTTCCCCCCA | |
| | AAGTAGAGATGCTAAGCCAAATCCAGAAGAACCCATAGATGAAGATGAAGATATTCAA | |
| | ACAGAAAGAATAAGAACAGCCACTGCTCTGACCACTTCAATCTTAGATGAGAAACCTG | |
| | TTATAATTGCCAGCTGTCTACACAAAGAATATGCAGGCCAGAAGAAAAGTTGCTTTTC | |
| | AAAGAGGAAGAAGAAAATAGCAGCAAGAAATATCTCTTTCTGTGTTCAAGAAGGTGAA | |
| | ATTTTGGGATTGCTAGGACCCAATGGTGCTGGAAAAAGTTCATCTATTAGAATCATAT | |
| | CTGGGATCACAAAGCCAACTGCTGGAGAGGTAGTGCTGAAAGGCTGCAGTTCAGTTTT | |
| | GGGCCACCTGGGGTACTGCCCTCAAGAGAACGTGCTGTGGCCCATGCTGACGTTGAGG | |
| | GAACACCTGGAGGTGTATGCTGCCGTCAAGGGGCTCAGGAAAGCGCACGCGAGGCTCG | |
| | CCATCGCAAGGAGATTAGTGAGTGCTTTCAAACTGCATGAGCAGCTGAATGTTCCTGT | |
| | GCAGAAATTAACAGCAGGAATCACGAGAAAGTTGTGTTTTGTGCTGAGCCTCCTGGGA | |
| | AACTCACCTGTCTTGCTCCTGGATGAACCATCTACGGGCATAGACCCCACAGGGCAGC | |
| | AGCAAATGTGGCAGGCAATCCAGGCAGTCGTTAAAAACACAGAGACAGGTGTCCTCCT | |
| | GACCACCCATAACCTGGCTGAGGCGGAAGCCTTGTGTGACCGTGTGGCCATCATGGTG | |
| | TCTGGAAGGCTTAGATGCATTGGCTCCATCCAACACCTGAAAAACAAACTTGGCAAGG | |
| | ATTACATTCTAGAGCTAAAAGTGAAGGAAACGTCTCAAGTGACTTTGGTCCACACTGA | |
| | GATTCTGAAGCTTTTCCCACAGGCTGCAGGGCAGGAAAGGTATTCCTCTTTGTTAACC | |
| | TATAAGCTGCCCGTGGCAGACGTTTACCCTCTATCACAGACCTTTCACAAATTAGAAG | |
| | CAGTGAAGCATAACTTTAACCTGGAAGAATACAGCCTTTCTCAGTGCACACTGGAGAA | |
| | GGTATTCTTAGAGCTTTCTAAAGAACAGGAAGTAGGAAATTTTGATGAAGAAATTGAT | |
| | ACAACAATGAGATGGAAACTCCTCCCTCATTCAGATGAACCTTAAAACCTCAAACCTA | |
| | GTAATTTTTT | |
| | ORF Start: ATG at 13 | ORF Stop: TAA at 4741 |
| | SEQ ID NO:200 | 1576 aa MW at 179721.1 kD |

| | |
|---|---|
| NOV47a, | MNMKQKSVYQQTKALLCKNFLKKWRMKRESLLEWGLSILLGLCIALFSSSMRNVQFPG |
| CG59909-01 Protein | MAPQNLGRTDKFNSSSLMVVYTPISNLTQQIMNKTALAPLLKGRSVIGAPNKTHMDEI |
| Sequence | LLENLPYAMGIIFNETFSYKLIFFQGYNSPLWKEDFSGTAHCWDGYGEFSCTLTKYWN |
| | RGFVALQTAINTAIIEITTNHPVMEELMSVTAITMKTLPFITKNLLHNEMFILFFLLH |
| | FSPLVYFISLNVTKERKKSKNLMKMMGLQDSAFWLSWGLIYAGFIFIISIFVTIIITF |
| | TQIIVMTGFMVIFILFFLYGLSLVALVFLMSVLLKKAVLTNLVVFLLTLFWGCLGFTV |
| | FYEQLPSSLEWILNICSPFAFTTGMIQVIKLDYNLNGVIFPDPSGDSYTMIATFSMLL |
| | LDGLIYLLLALYFDKILPCKDERHYSPLFFLNSSSCFQHQRTNAKVIEKEIDAEHPSD |
| | DYFEPVAPEFQCKEAIRIRNVKKEYKGKSGKVEALKGLLFDIYEGQITAILGHSGAGK |
| | SSLLNILNGLSVPTEGSVTIYNKNLSEMQDLEEIRKITGVCPQFNVQFDILTVKENLS |
| | LFAKIKGIHLKEVEQEILLLDEPTTGLDPFSRDQVWSLLRERRADHVILFSTQSMDEA |
| | DILADRKVIMSNGRLKCAGSSMFLKRRWGLGYHLSLHRNEICNPEQITSFITHHIPDA |
| | KLKTENKEKLVYTLPLERTNTFPDLFSDLDKCSDQCVTGYDISMSTLNEVFMKLEGQS |
| | TIEQGKAICIIHRQVEMIRDSESLNEMELAHSSFSEMQTAVSDMGLWRMQVFAMARLR |
| | FLKLKRQTKVLLTLLLVFGIAIFPLIVENIMYAMLNEKIDWEFKNELYFLSPGQLPQE |
| | PRTSLLIINNTGSNIEDFIKSLKHQNILLEVDDFENRNGTDGLSYNGAIIVCFCFKDY |
| | RFSVVCNTKRLHCFPILMNIISNGLLQMFNHTQHIRIESSPFSHIGLWTGLPDGSFFL |
| | FLVLCSISPYITMCSISDYKVRAKSQLWISGLYTSAYWCGQALVDVSFFILILLLMYL |
| | IFYIENMQYLLITYFFYAQVIVTPGYAASLVFFIYMISFIFRKRRKNSGLWSFYFFFA |
| | STIMFSITLINHFDLSILITTMVLVPSYTLLGFKTFLEVRDQEHYRELSATDFLVCFI |
| | PYFQTLLFVFVLRCMELKCGKKRMRKDPVFRISPQSRDAKPNPEEPIDEDEDIQTERI |
| | RTATALTTSILDEKPVIIASCLHKEYAGQKKSCFSKRKKKIAARNISFCVQEGEILGL |
| | LGPNGAGKSSSIRMISGITKPTAGEVVLKGCSSVLGHLGYCPQENVLWPMLTLREHLE |
| | VYAAVKGLRKADARLAIARRLVSAFKLHEQLNVPVQKLTAGITRKLCFVLSLLGNSPV |
| | LLLDEPSTGIDPTCQQQMWQAIQAVVKNTERGVLLTTHNLAEAEALCDRVAIMVSGRL |
| | RCIGSIQHLKNKLGKDYILELKVKETSQVTLVHTEILKLFPQAAGQERYSSLLTYKLP |
| | VADVYPLSQTFHKLEAVKHNFNLEEYSLSQCTLEKVFLELSKEQEVGNFDEEIDTTMR |
| | WKLLPHSDEP |

Further analysis of the NOV47a protein yielded the following properties shown in Table 47B.

TABLE 47B
Protein Sequence Properties NOV47a

| | |
|---|---|
| PSort | 0.8000 probability located in plasma membrane; 0.4000 |
| analysis: | probability located in Golgi body; 0.3000 probability located |
| | in endoplasmic reticulum (membrane); 0.3000 probability |
| | located in microbody (peroxisome) |
| SignalP | Cleavage site between residues 51 and 52 |
| analysis: | |

A search of the NOV47a protein against the Geneseq database, a proprietary database that contains sequences published in patents and patent publication, yielded several homologous proteins shown in Table 47C.

TABLE 47C
Geneseq Results for NOV47a

| | | NOV47a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAU18484 | Human endocrine polypeptide SEQ ID | 1229 . . . 1526 | 294/298 (98%) | e−164 |
| | No 439 - Homo sapiens, 310 aa. | 13 . . . 309 | 294/298 (98%) | |
| | [WO200155364-A2, 02-AUG-2001] | | | |
| AAE09582 | Human gene 2 encoded ABC | 1229 . . . 1526 | 294/298 (98%) | e−164 |
| | transporter protein HDPVY34, SEQ ID | 13 . . . 309 | 294/298 (98%) | |
| | NO: 24 - Homo sapiens, 310 aa. | | | |
| | [WO200155208-A1, 02-AUG-2001] | | | |
| AAM42360 | Human polypeptide SEQ ID NO 93 - | 1229 . . . 1526 | 294/298 (98%) | e−164 |
| | Homo sapiens, 310 aa. | 13 . . . 309 | 294/298 (98%) | |
| | [WO200155449-A1, 02-AUG-2001) | | | |
| AAM93187 | Human polypeptide, SEQ ID NO: 2559 - | 373 . . . 815 | 273/483 (56%) | e−149 |
| | Homo sapiens, 483 aa. [EP1130094- | 1 . . . 483 | 339/483 (69%) | |
| | A2, 05-SEP-2001] | | | |
| AAB67309 | ABC transport related protein #3 - | 1216 . . . 1574 | 256/361 (70%) | e−146 |
| | Homo sapiens, 362 aa. | 2 . . . 361 | 303/361 (83%) | |
| | [WO200107658-A1, 01-FEB-2001] | | | |

In a BLAST search of public sequence databases, the NOV47a protein was found to have homology to the proteins shown in the BLASTP data in Table 47D.

TABLE 47D
Public BLASTP Results for NOV47a

| | | Residues/ | | |
|---|---|---|---|---|
| Accession | | Match | Similarities for the | |
| Number | Protein/Organism/Length | Residues | Matched Portion | Value |
| AAK30023 | ATP-BINDING CASSETTE A6 - | 1 . . . 1576 | 1544/1626 (94%) | 0.0 |
| | Homo sapiens (Human), 1617 aa. | 1 . . . 1617 | 1549/1626 (94%) | |
| O94911 | KIAA0822 PROTEIN - Homo | 1 . . . 1574 | 929/1581 (58%) | 0.0 |
| | sapiens (Human), 1581 aa. | 1 . . . 1580 | 1149/1581 (71%) | |
| AAK30025 | ATP-BINDING CASSETTE A10 - | 90 . . . 1574 | 922/1543 (59%) | 0.0 |
| | Homo sapiens (Human), 1543 | 1 . . . 1542 | 1126/1543 (72%) | |
| | aa. | | | |
| AAK30024 | ATP-BINDING CASSETTE A9 - | 1 . . . 1570 | 917/1620 (56%) | 0.0 |
| | Homo sapiens (Human), 1624 aa. | 1 . . . 1619 | 1162/1620 (71%) | |
| AAK30022 | ATP-BINDING CASSETTE A5 - | 3 . . . 1568 | 653/1651 (39%) | 0.0 |
| | Homo sapiens (Human), 1642 aa. | 5 . . . 1633 | 969/1651 (58%) | |

PFam analysis indicates that the NOV47a protein contains the domains shown in the Table 47E.

TABLE 47E
Domain Analysis of NOV47a

| | | Identities/ | |
|---|---|---|---|
| | NOV47a | Similarities | |
| | Match | for the Matched | Expect |
| Pfam Domain | Region | Region | Value |
| Peptidase_S26: domain 1 | 1 . . . 46 | 14/47 (30%) | 7.9 |
| of 1 | | 35/47 (74%) | |
| Virus_HS: domain 1 of 1 | 169 . . . 174 | 4/6 (67%) | 2.1 |
| | | 6/6 (100%) | |
| ABC_tran: domain 1 of 2 | 509 . . . 651 | 58/199 (29%) | 4.7e−26 |
| | | 117/199 (59%) | |
| 7tm_1: domain 1 of 1 | 1053 . . . 1085 | 6/34 (18%) | 0.46 |
| | | 25/34 (74%) | |
| ABC_tran: domain 2 of 2 | 1271 . . . 1448 | 59/199 (30%) | 8.7e−44 |
| | | 137/199 (69%) | |

### Example 48

The NOV48 clone was analyzed, and the nucleotide and polypeptide sequences are shown in Table 48A.

TABLE 48A
NOV48 Sequence Analysis

| | SEQ ID NO:201 | 1599 bp |
|---|---|---|
| NOV48a, | CGCAGCAGTTTGTGTTCCCTGATGGAAAATGAAGCCTCATCTACTGACTCTCCCATCC | |
| CG59945-01 DNA | AAAAAGACTTGAGGGCAAACGAGGACAAAAGTTCCCACGAGGTGGGCACTGCGGGAAG | |
| Sequence | GAGGCCCACGACCCCGAGATTCTCCGGCGACCCCGGCGGGCCACCCCGGGCCGGTGAG | |
| | GGTTCCCGCGGCACGTGTGCGGCGGCGCCCGCCGCTTCCCCCGCCGCCCGGGAAGGTC | |
| | ACGCCGCCCGGACGCCGAGGCTCTCCCCGTGCCCCGCTCCCTCCGCCCCGCACACTCC | |
| | CGTGACCAGCCCCATCTCCAGCCAGGTGGTGGGCAGTGAGCCACTACAGTACATCATG | |
| | GCAGAGCCGGCCAGGCCTGACAGTCCAAAGGGCTCCTCGGAGACAGAGACCGAGCCTC | |
| | CTGTGGCCCTGGCCCCTGGTCCAGCTCCCACCCACTGCCTCCCAGGCCACAAGGAAGA | |
| | GGAGGATGGGGAGGGGGCTGGGCCTGGCGAGCAGGGCGGTGGGAAGCTGGTGCTCAGC | |
| | TCCCTGTCCAAGCGCCTCTGCCTGGTCTGTGGGGACGTGGCCTCCGGCTACCACTGCG | |
| | GTGTGTCATCCTGTGAGGACTGCAAAGCCTTCTTCAAGAGGACCATCCAGCCCAGTAT | |
| | GGAGTACAGCTGTCTGGCCTCCAACGAGTGTGAGATCACCAAGCGGAGACGCAACCCC | |
| | TGTCAGGCCTGCCGCTTCACCAAGAGCCTGCGGCTTCACCAAGAGCCTGCGGGAGCCC | |
| | GCCTGGACCGCGTCCGGGGTGGGCGGGAGTACAAGCCGTGCCCAGAGGTGGACCCGCT | |
| | GCCCTTCCCGGGCGCCTTCCCTGCTGGGCCCCTGGCAGTCGCTGGAGGCCCCCAGACG | |
| | ACAGGCCCAGTGAATGCACTGGTGTCTCATCTAATGGTGGTTGAGCCTGAGAAGCTCT | |
| | ATGCCTTGCCCGACCCTGCTGGCCCTGATGGGCACCTTCCAGCCGTGGCTACCCTCTG | |
| | TGACCTCTTTGACCGAGAGATCGTGGTCACCATCAGCTGGGCCAAGAGCATCCCACGC | |
| | TTCTCATCGCTGTCGCTGTCTGACCAGATGTCAGTACTGCAGAGAGTATGGATGGAGG | |
| | TGCTGGTGCCGGGTGTGGCCCAGCGCTCACTGCCACTGCAGGATGAGTTGGCCTTCGC | |
| | TGAGGACTTAGTCCTGGATGAACAGGGGGCACGGGCAGCTGGCCTGGGGGAACTGGGG | |
| | GCTGCCCTGCTGCAACTGGTGCGGCGGCTGCAGTCCCTGCGGCTGGAGCGAGGGGAGT | |
| | ACGTTCTACTGAAGGCCCTGGCCCTTGCCAATTCAGACTCTGTGCCCATCGAAGATGC | |
| | CGAGGCTGTGGAGCAGCTGCCAGAAGCTCCGCACGAGGCCCTGCTGGAGTATGAAGCC | |
| | GGCCGAGCTGGCACCGGAGGGGGTGCTGAGCGGCGGCGGCCACGCAGGCTGCTGTTCA | |
| | CGCTACCGCTCCTCCACCAGACAGCGGGCAAAGTGCTGGCCCATTTCTATGGGGTGAA | |
| | GCTGGAGGGCAAGGTGCCCATGCACAAGCTGTTCTTGGAGATGCTCGAGGCCATGATG | |
| | GACTGA+E, UNS GGCGAGGGGTGGGACTGGTGGGGGTTC | |
| | ORF Start: ATG at 22 | ORF Stop: TGA at 1570 |
| | SEQ ID NO:202 | 516 aa MW at 54706.5 kD |

| | |
|---|---|
| NOV48a, | MENEASSTDSPIQKDLRAKEDKSSHEVGTAGRRPTTPRFSGDPGGPPRGGEGSRGTCA |
| CG59945-01 Protein | AAPAASPAAREGHAARTPRLSPCPAPSAPHTPVTSAMSSQVVGSEPLQYIMAEPARPD |
| Sequence | SPKGSSETETEPPVALAPGPAPTHCLPGHKEEEDGEGAGPGEQGGGKLVLSSLSKRLC |
| | LVCGDVASCYHCCVSSCEDCKAFFKRTIQGSMEYSCLASNECEITKRRRKACQACRFT |
| | KSLRLHQEPAGARLDRVRGGREYKRCPEVDPLPFPGAFPAGPLAVAGGPQTTGPVNAL |
| | VSHLMVVEPEKLYALPDPAGPDGHLPAVATLCDLFDREIVVTISWAKSIPGFSSLSLS |
| | DQMSVLQRVWMEVLVPGVAQRSLPLQDELAFAEDLVLDEEGARAAGLGELGAALLQLV |
| | RRLQSLRLERGEYVLLKALALANSDSVPTEDAEAVEQLPEAPHEALLEYEAGRAGTGG |
| | GAERRRPGRLLFTLPLLHQTAGKVLAHFYGVKLEGKVPMHKLFLEMLEAMMD |

Further analysis of the NOV48a protein yielded the following properties shown in Table 48B.

TABLE 48B
Protein Sequence Properties NOV48a

| | |
|---|---|
| PSort | 0.7000 probability located in nucleus; 0.3000 probability |
| analysis: | located in microbody (peroxisome); 0.1000 probability located |
| | in mitochondrial matrix space; 0.1000 probability located in |
| | lysosome (lumen) |
| SignalP | No Known Signal Sequence Indicated |
| analysis: | |

A search of the NOV48a protein against the Geneseq database, a proprietary database that contains sequences published in patents and patent publication, yielded several homologous proteins shown in Table 48C.

TABLE 48C
Geneseq Results for NOV48a

| | | NOV48a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAM39411 | Human polypeptide SEQ ID NO 2556 - | 4 . . . 516 | 418/520 (80%) | 0.0 |
| | Homo sapiens, 552 aa. | 36 . . . 552 | 432/520 (82%) | |
| | [WO200153312-A1, 26-JUL-2001] | | | |
| AAP80930 | Sequence of human estrogen-related | 4 . . . 516 | 409/521 (78%) | 0.0 |
| | receptor protein (hERR1) - Homo | 4 . . . 521 | 425/521 (81%) | |
| | sapiens, 521 aa. [WO8803168-A, 05- | | | |
| | MAY-1988] | | | |
| AAM41197 | Human polypeptide SEQ ID NO 6128 - | 4 . . . 516 | 406/521 (77%) | 0.0 |
| | Homo sapiens, 556 aa. | 39 . . . 556 | 422/521 (80%) | |
| | [WO200153312-A1, 26-JUL-2001] | | | |
| AAY83822 | Human nuclear receptor nNR1 - Homo | 100 . . . 513 | 227/438 (51%) | e−115 |
| | sapiens, 500 aa. [US6054295-A, 25- | 8 . . . 431 | 287/438 (64%) | |
| | APR-2000] | | | |
| AAB12970 | Human oestrogen related receptor 4 | 100 . . . 513 | 227/438 (51%) | e−115 |
| | (ERR4) protein sequence - Homo | 8 . . . 431 | 287/438 (64%) | |
| | sapiens, 433 aa. [WO200042180-A1, | | | |
| | 20-JUL-2000] | | | |

In a BLAST search of public sequence databases, the NOV48a protein was found to have homology to the proteins shown in the BLASTP data in Table 48D.

TABLE 48D
Public BLASTP Results for NOV48a

| | | NOV48a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P11474 | Steroid hormone receptor ERR1 | 4 . . . 516 | 418/519 (80%) | 0.0 |
| | (Estrogen-related receptor, alpha) (ERR- | 4 . . . 519 | 432/519 (82%) | |
| | alpha) (Estrogen receptor-like 1) - Homo | | | |
| | sapiens (Human), 519 aa. | | | |
| A29345 | precursor - human, 521 aa. | 4 . . . 521 | 425/521 (81%) | 0.0 |
| E1262508 | SEQUENCE 18 FROM PATENT | 4 . . . 513 | 404/518 (77%) | 0.0 |
| | WO9709348 - Homo sapiens (Human), | 4 . . . 518 | 421/518 (80%) | |
| | 518 aa. | | | |
| O08580 | Steroid hormone receptor ERR1 | 66 . . . 516 | 393/463 (84%) | 0.0 |
| | (Estrogen-related receptor, alpha) (ERR- | 2 . . . 462 | 404/463 (86%) | |
| | alpha) (Estrogen receptor-like 1) - Mus | | | |
| | musculus (Mouse), 462 aa (fragment). | | | |
| Q96102 | UNKNOWN (PROTEIN FOR | 199 . . . 516 | 289/320 (90%) | e−158 |
| | IMAGE: 4301880) - Homo sapiens | 4 . . . 323 | 296/320 (92%) | |
| | (Human), 323 aa (fragment). | | | |

PFam analysis indicates that the NOV48a protein contains the domains shown in the Table 48E.

TABLE 48E
Domain Analysis of NOV48a

| | | Identities/ | |
|---|---|---|---|
| | | Similarities | |
| | NOV48a Match | for the Matched | Expect |
| Pfam Domain | Region | Region | Value |
| zf-C4: domain 1 of 1 | 172 . . . 233 | 35/63 (56%) | 9.3e−36 |
| | | 58/63 (92%) | |
| hormone_rec: domain 1 | 326 . . . 511 | 58/209 (28%) | 2.2e−30 |
| of 1 | | 136/209 (65%) | |

### Example B

Identification of NOVX Clones

The novel NOVX target sequences identified in the present invention may have been subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus.

Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2. 1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

### Example C

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune diseases), Panel CNSD.01 (containing central nervous system samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl . sscDNA samples are then normalized to reference nucleic acids as described previously, using 1× TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1× TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff pl effusion=pleural effusion,

glio=glioma,

astro=astrocytoma, and

neuro=neuroblastoma.

General_Screening_Panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D)/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μnon essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethaniol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10volume of 3M sodium acetate and 2 volumes of 100% ethanol. The RNA was spun down and placed in RNAse free water. RNA was stored at −80° C.

AI_Comprehensive Panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22 /Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

| | |
|---|---|
| Patient 2 | Diabetic Hispanic, overweight, not on insulin |
| Patient 7-9 | Nondiabetic Caucasian and obese (BMI > 30) |
| Patient 10 | Diabetic Hispanic, overweight, on insulin |
| Patient 11 | Nondiabetic African American and overweight |
| Patient 12 | Diabetic Hispanic on insulin |

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 51 contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 51.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathlology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; patient not demented but showing sever AD-like pathology

SupTemporal Ctx=Superior Temporal Cortex

Inf Temporal Ctx=Inferior Temporal Cortex

### A. NOV1a (CC58546-01: ADILICAN)

Expression of gene CG58546-01 was assessed using the primer-probe sets Ag2933, Ag3370 and Ag3837, described in Tables AA, AB and AC.

TABLE AA
Probe Name Ag2933

| | | | | Start |
|---|---|---|---|---|
| Primers | Sequences | | Length | Position |
| Forward | 5′-caccaccactaagccagaac-3′ | (SEQ ID NO:203) | 20 | 4546 |
| Probe | TET-5′-ttctcagtccaagaacatctcaaatatgt-3′-TAMRA | (SEQ ID NO:204) | | |
| Reverse | 5′-ggattccccatgtaattcaag-3′ | (SEQ ID NO:205) | 21 | 4618 |

TABLE AB
Probe Name Ag3370

| | | | | Start |
|---|---|---|---|---|
| Primers | Sequences | | Length | Position |
| Forward | 5′-agctggattcttccaaacaga-3′ | (SEQ ID NO:206) | 21 | 1844 |
| Probe | TET-5′-tcacatgtatacatgctgccaaatgg-3′-TAMRA | (SEQ ID NO: 207) | 26 | 1892 |
| Reverse | 5′-acctttgggatggaaagagtt-3′ | (SEQ ID NO:208) | 21 | 1918 |

TABLE AC
Probe Name Ag3837

| | | | | Start |
|---|---|---|---|---|
| Primers | Sequences | | Length | Position |
| Forward | 5′-acgagcttgaggatgtggat-3′ | (SEQ ID N0:209) | 20 | 4260 |
| Probe | TET-5′-ttttgtcctctgtgacagtctccaca-3′-TAMRA | (SEQ ID NO:210) | 26 | 4293 |
| Reverse | 5′-gcttcttcctggtgaaatgg-3′ | (SEQ ID N0:211) | 20 | 4319 |

CNS_neurodegeneration_v1.0 Summary: Ag2933/Ag3370 Expression of the CG58546-01 gene is low/undetectable (CTs>35) across all of the samples on this panel.

General_screening_panel_v1.4 Summary: Ag3370/Ag3837 The amp plots suggest that there were experimental difficulties with these runs.

Panel 1.3D Summary: Ag2933 Expression of the CG58546-01 gene is low/undetectable (CTs>35) across all of the samples on this panel.

### B. NOV2a and NOV2b (CG58598-01 and CG58598-02: BRAIN-SPECIFIC TM PROTEIN)

Expression of genes CG58598-01 and CG58598-02 was assessed using the primer-probe set Ag3383, described in Table BA. Results of the RTQ-PCR runs are shown in Tables BB, BC, and BD. Please note that CG58598-02 represents a full-length physical clone of the CG58598-01 gene, validating the prediction of the gene sequence.

TABLE BA
Probe Name Ag3383

| | | | | Start |
|---|---|---|---|---|
| Primers | Sequences | | Length | Position |
| Forward | 5′-ttcatqggagtgaggatagttg-3′ | (SEQ ID NO:212) | 22 | 367 |
| Probe | TET-5′-ctgcaataatggcaattaccggcatt-3′-TAMRA | (SEQ ID NO:213) | 26 | 389 |
| Reverse | 5′-cgtggaaattatctgcatatgc-3′ | (SEQ ID NO:214) | 22 | 424 |

TABLE BB
CNS_neurodegeneration_v1.0

| | Rel. Exp.(%) Ag3383, | | Rel. Exp.(%) Ag3383, |
|---|---|---|---|
| Tissue Name | Run 210154822 | Tissue Name | Run 210154822 |
| AD 1 Hippo | 0.0 | Control (Path) 3 | 0.0 |
| | | Temporal Ctx | |
| AD 2 Hippo | 15.9 | Control (Path) 4 | 15.2 |
| | | Temporal Ctx | |
| AD 3 Hippo | 1.8 | AD 1 Occipital Ctx | 8.1 |
| AD 4 Hippo | 0.0 | AD 2 Occipital Ctx | 0.0 |
| | | (Missing) | |
| AD 5 Hippo | 80.7 | AD 3 Occipital Ctx | 2.2 |
| AD 6 Hippo | 3.0 | AD 4 Occipital Ctx | 11.9 |
| Control 2 Hippo | 26.2 | AD 5 Occipital Ctx | 40.9 |
| Control 4 Hippo | 4.4 | AD 6 Occipital Ctx | 11.9 |
| Control (Path) 3 | 0.0 | Control 1 Occipital | 0.0 |
| Hippo | | Ctx | |
| AD 1 Temporal Ctx | 0.0 | Control 2 Occipital | 100.0 |
| | | Ctx | |
| AD 2 Temporal Ctx | 8.4 | Control 3 Occipital | 7.2 |
| | | Ctx | |
| AD 3 Temporal Ctx | 0.0 | Control 4 Occipital | 0.0 |
| | | Ctx | |
| AD 4 Temporal Ctx | 3.3 | Control (Path) 1 | 45.4 |
| | | Occipital Ctx | |
| AD 5 Inf Temporal | 13.6 | Control (Path) 2 | 3.5 |
| Ctx | | Occipital Ctx | |
| AD 5 Sup Temporal | 6.5 | Control (Path) 3 | 0.0 |
| Ctx | | Occipital Ctx | |
| AD 6 Inf Temporal | 8.5 | Control (Path) 4 | 4.4 |
| Ctx | | Occipital Ctx | |
| AD 6 Sup Temporal | 0.0 | Control 1 Parietal | 0.0 |
| Ctx | | Ctx | |
| Control 1 Temporal | 0.0 | Control 2 Parietal | 7.5 |
| Ctx | | Ctx | |
| Control 2 Temporal | 24.1 | Control 3 Parietal | 12.9 |
| Ctx | | Ctx | |
| Control 3 Temporal | 9.3 | Control (Path) 1 | 28.1 |
| Ctx | | Parietal Ctx | |
| Control 3 Temporal | 0.0 | Control (Path) 2 | 9.2 |
| Ctx | | Parietal Ctx | |
| Control (Path) 1 | 21.6 | Control (Path) 3 | 0.0 |
| Temporal Ctx | | Parietal Ctx | |
| Control (Path) 2 | 8.5 | Control (Path) 4 | 19.8 |
| Temporal Ctx | | Parietal Ctx | |

TABLE BC
General_screening_panel_v1.4

| | Rel. Exp.(%) Ag3383, | | Rel. Exp.(%) Ag3383, |
|---|---|---|---|
| Tissue Name | Run 213496699 | Tissue Name | Run 213496699 |
| Adipose | 0.0 | Renal ca. TK-10 | 0.0 |
| Melanoma* | 0.0 | Bladder | 0.3 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.0 |
| MEL-5 | | | |
| Squamous cell | 0.2 | Colon ca.* (SW480 | 0.0 |
| carcinoma SCC-4 | | met) SW620 | |
| Testis Pool | 2.8 | Colon ca. HT29 | 0.0 |
| Prostate ca.* (bone | 0.3 | Colon ca. HCT-116 | 0.0 |
| met) PC-3 | | | |
| Prostate Pool | 5.7 | Colon ca. CaCo-2 | 0.0 |
| Placenta | 0.0 | Colon cancer tissue | 0.0 |
| Uterus Pool | 0.3 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 3.8 |
| Ovarian ca. OVCAR-5 | 0.0 | Small Intestine Pool | 0.0 |
| Ovarian ca. IGROV-1 | 0.6 | Stomach Pool | 0.0 |
| Ovarian ca. OVCAR-8 | 0.0 | Bone Marrow Pool | 1.8 |
| Ovary | 0.0 | Fetal Heart | 0.0 |
| Breast ca. MCF-7 | 0.0 | Heart Pool | 1.4 |
| Breast ca. MDA- | 0.0 | Lymph Node Pool | 2.7 |
| MB-231 | | | |
| Breast ca. BT 549 | 0.2 | Fetal Skeletal Muscle | 0.4 |
| Breast ca. T47D | 0.0 | Skeletal Muscle Pool | 0.0 |
| Breast ca. MDA-N | 0.0 | Spleen Pool | 0.0 |
| Breast Pool | 3.3 | Thymus Pool | 0.7 |
| Trachea | 0.6 | CNS cancer (glio/astro) | 53.6 |
| | | U87-MG | |
| Lung | 0.0 | CNS cancer (glio/astro) | 0.0 |
| | | U-118-MG | |
| Fetal Lung | 0.0 | CNS cancer (neuro; met) | 0.0 |
| | | SK-N-AS | |
| Lung ca. NCI-N417 | 0.0 | CNS cancer (astro) SF- | 0.0 |
| | | 539 | |
| Lung ca. LX-1 | 3.4 | CNS cancer (astro) | 0.0 |
| | | SNB-75 | |
| Lung ca. NCI-H146 | 0.0 | CNS cancer (glio) SNB- | 0.0 |
| | | 19 | |
| Lung ca. SHP-77 | 54.0 | CNS cancer (glio) SF- | 0.3 |
| | | 295 | |
| Lung ca. A549 | 0.0 | Brain (Amygdala) Pool | 1.6 |
| Lung ca. NCI-H526 | 79.0 | Brain (cerebellum) | 100.0 |
| Lung ca. NCI-H23 | 0.0 | Brain (fetal) | 9.9 |
| Lung ca. NCI-H460 | 0.0 | Brain (Hippocampus) | 6.3 |
| | | Pool | |
| Lung ca. HOP-62 | 0.0 | Cerebral Cortex Pool | 4.6 |
| Lung ca. NCI-H522 | 0.0 | Brain (Substantia nigra) | 3.5 |
| | | Pool | |
| Liver | 0.0 | Brain (Thalamus) Pool | 5.6 |
| Fetal Liver | 0.0 | Brain (whole) | 15.4 |
| Liver ca. HepG2 | 0.0 | Spinal Cord Pool | 6.8 |
| Kidney Pool | 6.9 | Adrenal Gland | 0.0 |
| Fetal Kidney | 7.7 | Pituitary gland Pool | 0.4 |
| Renal ca. 786-0 | 0.0 | Salivary Gland | 0.0 |
| Renal ca. A498 | 0.0 | Thyroid (female) | 11.4 |
| Renal ca. ACHN | 26.4 | Pancreatic ca. CAPAN2 | 0.4 |
| Renal ca. UO-31 | 0.6 | Pancreas Pool | 2.8 |

TABLE BD
Panel CNS_1

| | Rel. Exp.(%) Ag3383, | | Rel. Exp.(%) Ag3383, |
|---|---|---|---|
| Tissue Name | Run 171787529 | Tissue Name | Run 171787529 |
| BA4 Control | 0.0 | BA17 PSP | 2.7 |
| BA4 Control2 | 12.1 | BA17 PSP2 | 0.0 |
| BA4 | 0.0 | Sub Nigra Control | 0.0 |
| Alzheimer's2 | | | |
| BA4 Parkinson's | 0.0 | Sub Nigra Control2 | 16.5 |
| BA4 | 15.5 | Sub Nigra | 4.1 |
| Parkinson's2 | | Alzheimer's2 | |
| BA4 | 8.2 | Sub Nigra | 25.5 |
| Huntington's | | Parkinson's2 | |
| BA4 | 0.0 | Sub Nigra | 30.4 |
| Huntington's2 | | Huntington's | |
| BA4 PSP | 0.0 | Sub Nigra | 0.0 |
| | | Huntington's2 | |
| BA4 PSP2 | 5.5 | Sub Nigra PSP2 | 0.0 |
| BA4 Depression | 0.0 | Sub Nigra | 0.0 |
| | | Depression | |
| BA4 | 0.0 | Sub Nigra | 9.5 |
| Depression2 | | Depression2 | |
| BA7 Control | 4.1 | Glob Palladus | 0.0 |
| | | Control | |
| BA7 Control2 | 4.1 | Glob Palladus | 5.8 |
| | | Control2 | |
| BA7 | 0.0 | Glob Palladus | 5.3 |
| Alzheimer's2 | | Alzheimer's | |
| BA7 Parkinson's | 0.0 | Glob Palladus | 0.0 |
| | | Alzheimer's2 | |
| BA7 | 100.0 | Glob Palladus | 18.3 |
| Parkinson's2 | | Parkinson's | |
| BA7 | 0.0 | Glob Palladus | 0.0 |
| Huntington's | | Parkinson's2 | |
| BA7 | 0.0 | Glob Palladus PSP | 3.7 |
| Huntington's2 | | | |
| BA7 PSP | 0.0 | Glob Palladus PSP2 | 0.0 |
| BA7 PSP2 | 6.2 | Glob Palladus | 0.0 |
| | | Depression | |
| BA7 Depression | 0.0 | Temp Pole Control | 0.0 |
| BA9 Control | 0.0 | Temp Pole Control2 | 0.0 |
| BA9 Control2 | 18.4 | Temp Pole | 0.0 |
| | | Alzheimer's | |
| BA9 Alzheimer's | 0.0 | Temp Pole | 0.0 |
| | | Alzheimer's2 | |
| BA9 | 5.4 | Temp Pole | 0.0 |
| Alzheimer's2 | | Parkinson's | |
| BA9 Parkinson's | 0.0 | Temp Pole | 0.0 |
| | | Parkinson's2 | |
| BA9 | 14.2 | Temp Pole | 5.0 |
| Parkinson's2 | | Huntington's | |
| BA9 | 0.0 | Temp Pole PSP | 0.0 |
| Huntington's | | | |
| BA9 | 0.0 | Temp Pole PSP2 | 0.0 |
| Huntington's2 | | | |
| BA9 PSP | 0.0 | Temp Pole | 0.0 |
| | | Depression2 | |
| BA9 PSP2 | 0.0 | Cing Gyr Control | 0.0 |
| BA9 Depression | 0.0 | Cing Gyr Control2 | 0.0 |
| BA9 | 0.0 | Cing Gyr Alzheimer's | 0.0 |
| Depression2 | | | |
| BA17 Control | 8.4 | Cing Gyr | 0.0 |
| | | Alzheimer's2 | |
| BA17 Control2 | 9.0 | Cing Gyr Parkinson's | 0.0 |
| BA17 | 0.0 | Cing Gyr | 0.0 |
| Alzheimer's2 | | Parkinson's2 | |
| BA17 | 5.7 | Cing Gyr | 0.0 |
| Parkinson's | | Huntington's | |
| BA17 | 17.3 | Cing Gyr | 0.0 |
| Parkinson's2 | | Huntington's2 | |
| BA17 | 12.0 | Cing Gyr PSP | 4.5 |
| Huntington's | | | |
| BA17 | 0.0 | Cing Gyr PSP2 | 0.0 |
| Huntington's2 | | | |
| BA17 | 0.0 | Cing Gyr Depression | 0.0 |
| Depression | | | |
| BA17 | 4.1 | Cing Gyr | 0.0 |
| Depression2 | | Depression2 | |

CNS_neurodegeneration_v1.0 Summary: Ag3383 This panel confirms the expression of the CG58598-01 gene at low levels in the brains of an independent group of individuals. However, no differential expression of this gene was detected between Alzheimer's diseased postmortem brains and those of non-demented controls in this experiment. The brain preferential expression of this novel protein in both this panel and panel 1.4 suggests that this protein product may be a drug target for the treatment of neurologic disorders.

General_screening_panel_v1.4 Summary: Ag3383 Expression of the CG58598-01 gene appears to be preferential to the brain, with the highest level of expression in the cerebellum (CT=30.2). Please see Panel CNS_neurodegeneration_v1.0 for discussion of utility in the central nervous system. There is also expression in the CNS cancer cell line U87-MG (CT=3.1) but no expression in any other CNS cancer cell line. Therefore, expression of this gene can be used to distinguish between the U87-MG cell line and other CNS cancer cell lilies on this panel.

Additionally, there is low to moderate expression of this gene in renal and lung cancer cell lines. Thus, expression of this gene could be used to differentiate between these samples and other samples on this panel and as a marker to detect the presence of cancer. Furthermore, therapeutic modulation of the expression or function of this gene may be effective in the treatment of these cancers.

Panel 2.2 Summary: Ag3383—Expression of the CG58598-01 gene is low/undetectable (CTs>35) across all of the samples on this panel.

Panel 4D Summary: Ag3383—Expression of the CG58598-01 gene is low/undetectable (CTs>35) across all of the samples on this panel.

Panel CNS_{—}1 Summary: Ag3383 Expression in this panel confirms expression of the CG58598-01 gene in the brain. Please see Panel CNS_neurodegeneration_v1.0 for discussion of utility in the central nervous system.

### C. NOV3a (CG57833-01: AMINO ACID TRANSPORTER)

Expression of gene CG57833-01 was assessed using the primer-probe set Ag3341, described in Table CA. Results of the RTRQ-PCR runs are shown in Tables CB and CC.

TABLE CA
Probe Name Ag3341

| | | | | Start |
|---|---|---|---|---|
| Primers | Sequences | | Length | Position |
| Forward | 5′-aacactaactgtgcccattgtc-3′ | (SEQ ID NO:215) | 22 | 1284 |
| Probe | TET-5′-tcccaattcgtacatcagtgatcaca-3′-TAMRA | (SEQ ID NO:216) | 26 | 1310 |
| Reverse | 5′-gaaatgtcgtatccagctgaag-3′ | (SEQ ID NO:217) | 22 | 1356 |

TABLE CB
Panel 1.3D

| | Rel. Exp.(%) Ag3341, | | Rel. Exp.(%) Ag3341, |
|---|---|---|---|
| Tissue Name | Run 165524268 | Tissue Name | Run 165524268 |
| Liver adenocarcinoma | 0.2 | Kidney (fetal) | 6.9 |
| Pancreas | 0.8 | Renal ca. 786-0 | 0.1 |
| Pancreatic ca. CAPAN 2 | 1.6 | Renal ca. A498 | 0.2 |
| Adrenal gland | 0.5 | Renal ca. RXF 393 | 0.4 |
| Thyroid | 0.1 | Renal ca. ACHN | 0.0 |
| Salivary gland | 0.0 | Renal ca. UO-31 | 0.1 |
| Pituitary gland | 0.3 | Renal ca. TK-10 | 0.0 |
| Brain (fetal) | 0.4 | Liver | 100.0 |
| Brain (whole) | 0.2 | Liver (fetal) | 54.3 |
| Brain (amygdala) | 0.0 | Liver ca. | 0.7 |
| | | (hepatoblast) HepG2 | |
| Brain (cerebellum) | 0.0 | Lung | 0.2 |
| Brain (hippocampus) | 0.1 | Lung (fetal) | 0.2 |
| Brain (substantia nigra) | 0.0 | Lung ca. (small cell) | 0.1 |
| | | LX-1 | |
| Brain (thalamus) | 0.3 | Lung ca. (small cell) | 0.0 |
| | | NCI-H69 | |
| Cerebral Cortex | 0.0 | Lung ca. (s.cell var.) | 0.1 |
| | | SHP-77 | |
| Spinal cord | 0.2 | Lung ca. (large | 0.6 |
| | | cell)NCI-H460 | |
| glio/astro U87-MG | 0.0 | Lung ca. (non-sm. | 0.0 |
| | | cell) A549 | |
| glio/astro U-118-MG | 1.6 | Lung ca. (non-s.cell) | 0.0 |
| | | NCI-H23 | |
| astrocytoma SW1783 | 3.2 | Lung ca. (non-s.cell) | 0.8 |
| | | HOP-62 | |
| neuro*; met SK-N-AS | 0.1 | Lung ca. (non-s.cl) | 0.1 |
| | | NCI-H522 | |
| astrocytoma SF-539 | 0.0 | Lung ca. (squam.) | 0.0 |
| | | SW 900 | |
| astrocytoma SNB-75 | 0.1 | Lung ca. (squam.) | 0.2 |
| | | NCI-H596 | |
| glioma SNB-19 | 0.0 | Mammary gland | 0.0 |
| glioma U251 | 0.0 | Breast ca.* (pl.ef) | 0.0 |
| | | MCF-7 | |
| glioma SF-295 | 0.5 | Breast ca.* (pl.ef) | 5.3 |
| | | MDA-MB-231 | |
| Heart (fetal) | 0.0 | Breast ca.* (pl.ef) | 0.0 |
| | | T47D | |
| Heart | 0.0 | Breast ca. BT-549 | 0.0 |
| Skeletal muscle (fetal) | 0.3 | Breast ca. MDA-N | 0.0 |
| Skeletal muscle | 31.0 | Ovary | 0.2 |
| Bone marrow | 0.3 | Ovarian ca. OVCAR-3 | 0.5 |
| Thymus | 0.1 | Ovarian ca. OVCAR-4 | 0.0 |
| Spleen | 0.0 | Ovarian ca. OVCAR-5 | 3.0 |
| Lymph node | 0.5 | Ovarian ca. OVCAR-8 | 0.0 |
| Colorectal | 0.2 | Ovarian ca. IGROV-1 | 0.1 |
| Stomach | 0.0 | Ovarian ca.* (ascites) | 0.0 |
| | | SK-OV-3 | |
| Small intestine | 0.8 | Uterus | 2.0 |
| Colon ca. SW480 | 0.0 | Placenta | 0.0 |
| Colon ca.* | 0.1 | Prostate | 0.3 |
| SW620(SW480 met) | | | |
| Colon ca. HT29 | 0.0 | Prostate ca.* (bone | 0.0 |
| | | met)PC-3 | |
| Colon ca. HCT-116 | 0.0 | Testis | 0.0 |
| Colon ca. CaCo-2 | 10.2 | Melanoma | 1.1 |
| | | Hs688(A).T | |
| Colon ca. | 0.1 | Melanoma* (met) | 0.2 |
| tissue(ODO3866) | | Hs688(B).T | |
| Colon ca. HCC-2998 | 0.0 | Melanoma UACC-62 | 0.0 |
| Gastric ca.* (liver met) | 0.0 | Melanoma M14 | 0.0 |
| NCI-N87 | | | |
| Bladder | 10.2 | Melanoma LOX | 0.0 |
| | | IMVI | |
| Trachea | 0.3 | Melanoma* (met) | 0.0 |
| | | SK-MEL-5 | |
| Kidney | 0.7 | Adipose | 0.8 |

TABLE CC
Panel 4D

| | Rel. Exp.(%) | | Rel. Exp.(%) |
|---|---|---|---|
| | Ag3341, Run | | Ag3341, Run |
| Tissue Name | 165222714 | Tissue Name | 165222714 |
| Secondary Th1 act | 1.5 | HUVEC IL-1 beta | 0.0 |
| Secondary Th2 act | 0.0 | HUVEC IFN gamma | 0.8 |
| Secondary Tr1 act | 0.0 | HUVEC TNF alpha + IFN | 4.2 |
| | | gamma | |
| Secondary Th1 rest | 0.0 | HUVEC TNF alpha + IL4 | 0.8 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.6 | Lung Microvascular EC | 0.7 |
| | | TNFalpha + IL-1 beta | |
| Primary Th2 act | 1.4 | Microvascular Dermal EC | 9.7 |
| | | none | |
| Primary Tr1 act | 0.0 | Microsvasular Dermal EC | 0.5 |
| | | TNFalpha + IL-1 beta | |
| Primary Th1 rest | 0.0 | Bronchial epithelium | 14.5 |
| | | TNFalpha + IL1 beta | |
| Primary Th2 rest | 0.0 | Small airway epithelium | 1.0 |
| | | none | |
| Primary Tr1 rest | 0.0 | Small airway epithelium | 23.0 |
| | | TNFalpha + IL-1 beta | |
| CD45RA CD4 | 12.6 | Coronery artery SMC rest | 3.3 |
| lymphocyte act | | | |
| CD45RO CD4 | 0.9 | Coronery artery SMC | 0.0 |
| lymphocyte act | | TNFalpha + IL-1 beta | |
| CD8 lymphocyte act | 0.0 | Astrocytes rest | 0.0 |
| Secondary CD8 | 0.0 | Astrocytes TNFalpha + IL-1 | 0.0 |
| lymphocyte rest | | beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 3.0 |
| CD95 CH11 | | none | |
| LAK cells rest | 0.0 | CCD1106 (Keratinocytes) | 5.4 |
| | | TNFalpha + IL-1 beta | |
| LAK cells IL-2 | 0.9 | Liver cirrhosis | 100.0 |
| LAK cells IL-2 + IL-12 | 0.0 | Lupus kidney | 1.8 |
| LAK cells IL-2 + IFN | 0.0 | NCI-H292 none | 46.0 |
| gamma | | | |
| LAK cells IL-2 + IL-18 | 0.0 | NCI-H292 IL-4 | 72.7 |
| LAK cells | 0.0 | NCI-H292 IL-9 | 56.3 |
| PMA/ionomycin | | | |
| NK Cells IL-2 rest | 0.0 | NCI-H292 IL-13 | 39.2 |
| Two Way MLR 3 day | 0.0 | NCI-H292 IFN gamma | 87.1 |
| Two Way MLR 5 day | 0.0 | HPAEC none | 2.0 |
| Two Way MLR 7 day | 0.0 | HPAEC TNF alpha + IL-1 | 0.0 |
| | | beta | |
| PBMC rest | 0.0 | Lung fibroblast none | 14.4 |
| PBMC PWM | 0.8 | Lung fibroblast TNF | 2.5 |
| | | alpha + IL-1 beta | |
| PBMC PHA-L | 0.0 | Lung fibroblast IL-4 | 15.4 |
| Ramos (B cell) none | 0.0 | Lung fibroblast IL-9 | 11.3 |
| Ramos (B cell) | 0.0 | Lung fibroblast IL-13 | 12.0 |
| ionomycin | | | |
| B lymphocytes PWM | 0.0 | Lung fibroblast IFN | 21.2 |
| | | gamma | |
| B lymphocytes CD40L | 0.0 | Dermal fibroblast | 66.9 |
| and IL-4 | | CCD1070 rest | |
| EOL-1 dbcAMP | 0.0 | Dermal fibroblast | 28.3 |
| | | CCD1070 TNF alpha | |
| EOL-1 dbcAMP | 0.0 | Dermal fibroblast | 27.0 |
| PMA/ionomycin | | CCD1070 IL-1 beta | |
| Dendritic cells none | 0.0 | Dermal fibroblast IFN | 1.1 |
| | | gamma | |
| Dendritic cells LPS | 0.0 | Dermal fibroblast IL-4 | 3.2 |
| Dendritic cells anti- | 0.0 | IBD Colitis 2 | 0.0 |
| CD40 | | | |
| Monocytes rest | 0.0 | IBD Crohn's | 0.9 |
| Monocytes LPS | 0.0 | Colon | 6.9 |
| Macrophages rest | 0.0 | Lung | 1.4 |
| Macrophages LPS | 0.0 | Thymus | 40.9 |
| HUVEC none | 0.8 | Kidney | 6.9 |
| HUVEC starved | 3.9 | | |

Panel 1.3D Summary: Ag3341 The CG57833-01 gene codes for amino acid transporter HNAT3. Tissue distribution of this gene is similar to the results previously reported (Ref. 1) with highest expression of this gene in liver sample (CT=28.2). High expression of this gene is also detected in fetal liver (CT=29). Therefore, expression of this gene can be used to distinguish liver samples from other samples used in this panel. In addition, therapeutic modulation of the activity of this gene or its protein product, through the use of small molecule drugs, protein therapeutics or antibodies, might be beneficial in the treatment of liver related diseases such as liver cirrhosis.

Low expression of this gene is also seen in ovarian cancer OVCAR-5, colon cancer CaCo-2, astrocytoma SW1783, glio/astro U-118-MG, and pancreatic cancer CAPAN 2 cell lines (CTs=33-34). Therefore, therapeutic modulation of the activity of this gene or its protein product, through the use of small molecule drugs, protein therapeutics or antibodies, might be beneficial in the treatment of lung cancer or ovarian cancer.

Significant expression of this gene is also detected in skeletal muscle (CT=29.9). Interestingly, this gene is expressed at much higher levels in adult (CT=29.9) when compared to fetal skeletal Muscle (CT=36.6). This observation suggests that expression of this gene can be used to distinguish adult from fetal skeletal muscle. Furthermore, therapeutic modulation of the amino acid transporter encoded by this gene could be useful in treatment of muscle related diseases. More specifically, treatment of weak or dystrophic muscle with the protein encoded by this gene could restore muscle mass or function.

In addition, moderate expression of this gene is also detected in fetal kidney and bladder (CTs=31-32). Interestingly, this gene is expressed at much higher levels in fetal (CT=32) when compared to adult kidney (CT=35.3). Therefore, expression of this gene can be used to distinguish fetal from adult kidney. Furthermore, therapeutic modulation of the amino acid transporter encoded by this gene could be useful in treatment of renal diseases.

References.

1. Gu S, Adan-Rice D, Leach R J, Jaiang J X. (2001) A novel human amino acid transporter, hNAT3: cDNA cloning, chromosomal mapping, genomic structure, expression, and functional characterization. Genomics 74(3):262-72.

Panel 4D Summary: Ag3341 Highest expression of CG57833-01 is seen in liver cirrhosis samples (CT=31.3). The amino acid transporter encoded for by this gene could potentially allow cells within the liver to respond to specific microenvironmental signals. Therefore, therapies designed with the protein encoded for by this gene may potentially modulate liver function and play a role in the identification and treatment of inflammatory or autoimmune diseases which affect the liver including liver cirrhosis and fibrosis.

Significant expression of this gene is also seen in thymus, dermal fibroblast, lung fibroblast, NCI-H292 cell lines, and TNFalpha+IL-1 beta treated bronchial and small airway epithelium samples (CTs=32-34). Interestingly, this gene is expressed at much higher levels in TNFalpha+IL-1 beta treated small airway epithelium (CT=33.4) when compared to untreated cells (CT=37.9). This observation suggests that expression of this gene can be used to distinguish the cytokine treated from untreated small airway epithelium. Furthermore, modulation of the expression or activity of the protein encoded by this gene through the application of small molecule therapeutics may be useful in the treatment of asthma, COPD, psoriasis and emphysema.

### D. NOV4a, NOV4b, AND NOV4c (CG57853-01, CG57853-02, and CC57853-03: ILEAL SODIUM/BILE ACID COTRANSPORTER)

Expression of gene CG57853-01, CG57853-02, and CG57853-03 was assessed using the primer-probe sets Ag3350 and Ag2838, described in Tables DA and DB. Results of the RTQ-PCR runs are shown in Tables DC, DD and DE. Please note that CG57853-03 represents a full-length physical clone of the CG57853-01 gene, validating the prediction of the gene sequence.

TABLE DA
Probe Name Ag3350

| | | | | Start |
|---|---|---|---|---|
| Primer | Sequences | | Length | Position |
| Forward | 5′-cttagctcttgccgttattcag-3′ | (SEQ ID NO:218) | 22 | 938 |
| Probe | TET-5′-aggccaatttagcttctgtggctcct-3′-TAMRA | SEQ ID NO:219) | 26 | 979 |
| Reverse | 5′-catttcacatccagaacacatg-3′ | (SEQ ID NO:220) | 22 | 1016 |

TABLE DB
Probe Name Ag2838

| | | | | Start |
|---|---|---|---|---|
| Primers | Sequences | | Length | Position |
| Forward | 5′-tcttagctcttgccgttattca-3′ | (SEQ ID NO:221) | 22 | 937 |
| Probe | TET-5′-aggccaatttagcttctgtggctcct-3′-TAMRA | (SEQ ID NO:222) | 26 | 979 |
| Reverse | 5′-acatccagaacacatggctact-3′ | (SEQ ID NO:223) | 22 | 1010 |

TABLE DC
CNS_neurodegeneration_v1.0

| | Rel. Exp.(%) | | Rel. Exp.(%) |
|---|---|---|---|
| | Ag3350, Run | | Ag3350, Run |
| Tissue Name | 210141593 | Tissue Name | 210141593 |
| AD 1 Hippo | 5.7 | Control (Path) 3 | 0.4 |
| | | Temporal Ctx | |
| AD 2 Hippo | 12.1 | Control (Path) 4 | 57.8 |
| | | Temporal Ctx | |
| AD 3 Hippo | 3.2 | AD 1 Occipital Ctx | 18.7 |
| AD 4 Hippo | 0.9 | AD 2 Occipital Ctx | 0.0 |
| | | (Missing) | |
| AD 5 Hippo | 100.0 | AD 3 Occipital Ctx | 4.7 |
| AD 6 Hippo | 78.5 | AD 4 Occipital Ctx | 6.7 |
| Control 2 Hippo | 12.9 | AD 5 Occipital Ctx | 19.9 |
| Control 4 Hippo | 1.4 | AD 6 Occipital Ctx | 37.4 |
| Control (Path) 3 | 0.0 | Control 1 Occipital | 2.1 |
| Hippo | | Ctx | |
| AD 1 Temporal | 10.2 | Control 2 Occipital | 45.4 |
| Ctx | | Ctx | |
| AD 2 Temporal | 59.9 | Control 3 Occipital | 6.5 |
| Ctx | | Ctx | |
| AD 3 Temporal | 4.7 | Control 4 Occipital | 1.4 |
| Ctx | | Ctx | |
| AD 4 Temporal | 5.9 | Control (Path) 1 | 46.3 |
| Ctx | | Occipital Ctx | |
| AD 5 Inf | 95.3 | Control (Path) 2 | 8.3 |
| Temporal Ctx | | Occipital Ctx | |
| AD 5 Sup | 33.9 | Control (Path) 3 | 0.0 |
| Temporal Ctx | | Occipital Ctx | |
| AD 6 Inf | 66.0 | Control (Path) 4 | 6.6 |
| Temporal Ctx | | Occipital Ctx | |
| AD 6 Sup | 95.9 | Control 1 Parietal | 1.0 |
| Temporal Ctx | | Ctx | |
| Control 1 | 1.5 | Control 2 Parietal | 12.3 |
| Temporal Ctx | | Ctx | |
| Control 2 | 14.8 | Control 3 Parietal | 7.2 |
| Temporal Ctx | | Ctx | |
| Control 3 | 3.3 | Control (Path) 1 | 29.3 |
| Temporal Ctx | | Parietal Ctx | |
| Control 3 | 55.1 | Control (Path) 2 | 63.3 |
| Temporal Ctx | | Parietal Ctx | |
| Control (Path) 1 | 39.5 | Control (Path) 3 | 1.3 |
| Temporal Ctx | | Parietal Ctx | |
| Control (Path) 2 | 24.0 | Control (Path) 4 | 24.1 |
| Temporal Ctx | | Parietal Ctx | |

TABLE DD
General_screening_panel_v1.4

| | Rel. Exp.(%) | | Rel. Exp.(%) |
|---|---|---|---|
| | Ag3350, Run | | Ag3350, Run |
| Tissue Name | 216523465 | Tissue Name | 216523465 |
| Adipose | 1.8 | Renal ca. TK-10 | 28.7 |
| Melanoma* | 1.0 | Bladder | 4.2 |
| Hs688(A).T | | | |
| Melanoma* | 2.1 | Gastric ca. (liver | 100.0 |
| Hs688(B).T | | met.) NCI-N87 | |
| Melanoma* M14 | 3.8 | Gastric ca. | 11.7 |
| | | KATO III | |
| Melanoma* | 3.1 | Colon ca. SW-948 | 6.4 |
| LOXIMVI | | | |
| Melanoma* SK- | 3.3 | Colon ca. SW480 | 12.3 |
| MEL-5 | | | |
| Squamous cell | 4.2 | Colon ca.* (SW480 | 17.0 |
| carcinoma SCC-4 | | met) SW620 | |
| Testis Pool | 6.9 | Colon ca. HT29 | 13.7 |
| Prostate ca.* | 19.9 | Colon ca. HCT-116 | 10.4 |
| (bone met) PC-3 | | | |
| Prostate Pool | 3.4 | Colon ca. CaCo-2 | 48.0 |
| Placenta | 0.0 | Colon cancer tissue | 14.5 |
| Uterus Pool | 0.3 | Colon ca. SW1116 | 0.7 |
| Ovarian ca. | 14.3 | Colon ca. Colo-205 | 14.0 |
| OVCAR-3 | | | |
| Ovarian ca. | 12.4 | Colon ca. SW-48 | 16.0 |
| SK-OV-3 | | | |
| Ovarian ca. | 2.1 | Colon Pool | 2.6 |
| OVCAR-4 | | | |
| Ovarian ca. | 62.0 | Small Intestine Pool | 4.9 |
| OVCAR-5 | | | |
| Ovarian ca. | 1.8 | Stomach Pool | 3.2 |
| IGROV-1 | | | |
| Ovarian ca. | 2.7 | Bone Marrow Pool | 2.9 |
| OVCAR-8 | | | |
| Ovary | 3.4 | Fetal Heart | 2.5 |
| Breast ca. MCF-7 | 4.6 | Heart Pool | 0.9 |
| Breast ca. MDA- | 10.2 | Lymph Node Pool | 5.3 |
| MB-231 | | | |
| Breast ca. BT 549 | 14.1 | Fetal Skeletal | 4.3 |
| | | Muscle | |
| Breast ca. T47D | 62.4 | Skeletal Muscle | 2.0 |
| | | Pool | |
| Breast ca. | 1.6 | Spleen Pool | 1.4 |
| MDA-N | | | |
| Breast Pool | 3.7 | Thymus Pool | 1.9 |
| Trachea | 15.4 | CNS cancer | 2.0 |
| | | (glio/astro) U87-MG | |
| Lung | 3.4 | CNS cancer (glio/ | 3.6 |
| | | astro) U-118-MG | |
| Fetal Lung | 8.8 | CNS cancer (neuro; | 2.1 |
| | | met) SK-N-AS | |
| Lung ca. | 0.1 | CNS cancer (astro) | 3.8 |
| NCI-N417 | | SF-539 | |
| Lung ca. LX-1 | 17.3 | CNS cancer (astro) | 2.8 |
| | | SNB-75 | |
| Lung ca. | 2.9 | CNS cancer (glio) | 2.5 |
| NCI-H146 | | SNB-19 | |
| Lung ca. SHP-77 | 4.7 | CNS cancer (glio) | 6.0 |
| | | SF-295 | |
| Lung ca. A549 | 7.2 | Brain (Amygdala) | 0.8 |
| | | Pool | |
| Lung ca. | 8.2 | Brain (cerebellum) | 6.0 |
| NCI-H526 | | | |
| Lung ca. | 6.9 | Brain (fetal) | 8.0 |
| NCI-H23 | | | |
| Lung ca. | 7.6 | Brain | 3.5 |
| NCI-H460 | | (Hippocampus) Pool | |
| Lung ca. HOP-62 | 3.5 | Cerebral Cortex | 2.0 |
| | | Pool | |
| Lung ca. | 5.0 | Brain (Substantia | 0.8 |
| NCI-H522 | | nigra) Pool | |
| Liver | 0.8 | Brain (Thalamus) | 6.0 |
| | | Pool | |
| Fetal Liver | 45.1 | Brain (whole) | 2.5 |
| Liver ca. HepG2 | 20.0 | Spinal Cord Pool | 1.2 |
| Kidney Pool | 8.1 | Adrenal Gland | 0.6 |
| Fetal Kidney | 13.5 | Pituitary gland Pool | 1.8 |
| Renal ca. 786-0 | 7.2 | Salivary Gland | 1.0 |
| Renal ca. A498 | 4.2 | Thyroid (female) | 0.2 |
| Renal ca. ACHN | 3.8 | Pancreatic ca. | 6.3 |
| | | CAPAN2 | |
| Renal ca. UO-31 | 3.0 | Pancreas Pool | 3.5 |

TABLE DE
Panel 4D

| | Rel. Exp.(%) | | Rel. Exp.(%) |
|---|---|---|---|
| | Ag3350, Run | | Ag3350, Run |
| Tissue Name | 165222880 | Tissue Name | 165222880 |
| Secondary Th1 | 3.0 | HUVEC IL-1 beta | 0.0 |
| act | | | |
| Secondary Th2 | 4.1 | HUVEC IFN | 3.7 |
| act | | gamma | |
| Secondary Tr1 act | 3.5 | HUVEC TNF | 1.3 |
| | | alpha + IFN gamma | |
| Secondary Th1 | 0.0 | HUVEC TNF | 2.5 |
| rest | | alpha + IL4 | |
| Secondary Th2 | 2.2 | HUVEC IL-11 | 1.1 |
| rest | | | |
| Secondary Tr1 | 1.9 | Lung Microvascular | 4.6 |
| rest | | EC none | |
| Primary Th1 act | 4.0 | Lung Microvascular | 4.9 |
| | | EC TNF alpha + | |
| | | IL-1 beta | |
| Primary Th2 act | 3.2 | Microvascular | 3.2 |
| | | Dermal EC none | |
| Primary Tr1 act | 3.5 | Microvasular | 1.7 |
| | | Dermal EC | |
| | | TNF alpha + | |
| | | IL-1 beta | |
| Primary Th1 rest | 6.6 | Bronchial | 7.1 |
| | | epithelium TNF | |
| | | alpha + IL1 beta | |
| Primary Th2 rest | 3.8 | Small airway | 2.7 |
| | | epithelium none | |
| Primary Tr1 rest | 4.0 | Small airway | 25.7 |
| | | epithelium | |
| | | TNF alpha + | |
| | | IL-1 beta | |
| CD45RA CD4 | 4.0 | Coronery artery | 1.8 |
| lymphocyte act | | SMC rest | |
| CD45RO CD4 | 3.7 | Coronery artery | 1.1 |
| lymphocyte act | | SMC TNF alpha + | |
| | | IL-1 beta | |
| CD8 lymphocyte | 2.4 | Astrocytes rest | 3.3 |
| act | | | |
| Secondary CD8 | 2.9 | Astrocytes | 4.4 |
| lymphocyte rest | | TNF alpha + | |
| | | IL-1 beta | |
| Secondary CD8 | 2.1 | KU-812 (Basophil) | 12.3 |
| lymphocyte act | | rest | |
| CD4 lymphocyte | 0.6 | KU-812 (Basophil) | 26.1 |
| none | | PMA/ionomycin | |
| 2ry Th1/Th2/ | 3.7 | CCD1106 | 6.8 |
| Tr1_anti-CD95 | | (Keratinocytes) | |
| CH11 | | none | |
| LAK cells rest | 1.0 | CCD1106 | 2.9 |
| | | (Keratinocytes) | |
| | | TNF alpha + | |
| | | IL-1 beta | |
| LAK cells IL-2 | 3.4 | Liver cirrhosis | 7.4 |
| LAK cells IL-2 + | 3.3 | Lupus kidney | 1.9 |
| IL-12 | | | |
| LAK cells IL-2 + | 6.7 | NCI-H292 none | 7.7 |
| IFN gamma | | | |
| LAK cells IL-2 + | 4.5 | NCI-H292 IL-4 | 11.9 |
| IL-18 | | | |
| LAK cells | 1.4 | NCI-H292 IL-9 | 8.1 |
| PMA/ionomycin | | | |
| NK Cells IL-2 | 0.8 | NCI-H292 IL-13 | 6.6 |
| rest | | | |
| Two Way MLR 3 | 1.7 | NCI-H292 IFN | 8.7 |
| day | | gamma | |
| Two Way MLR 5 | 3.1 | HPAEC none | 3.5 |
| day | | | |
| Two Way MLR 7 | 1.0 | HPAEC TNF | 1.8 |
| day | | alpha + IL-1 beta | |
| PBMC rest | 0.3 | Lung fibroblast | 2.0 |
| | | none | |
| PBMC PWM | 14.5 | Lung fibroblast TNF | 1.1 |
| | | alpha + IL-1 beta | |
| PBMC PHA-L | 4.9 | Lung fibroblast IL-4 | 7.6 |
| Ramos (B cell) | 3.2 | Lung fibroblast IL-9 | 3.6 |
| none | | | |
| Ramos (B cell) | 13.2 | Lung fibroblast | 2.0 |
| ionomycin | | IL-13 | |
| B lymphocytes | 15.8 | Lung fibroblast | 2.9 |
| PWM | | IFN gamma | |
| B lymphocytes | 4.7 | Dermal fibroblast | 7.7 |
| CD40L and IL-4 | | CCD1070 rest | |
| EOL-1 dbcAMP | 4.6 | Dermal fibroblast | 6.6 |
| | | CCD1070 TNF | |
| | | alpha | |
| EOL-1 dbcAMP | 2.5 | Dermal fibroblast | 1.7 |
| PMA/ionomycin | | CCD1070 IL-1 beta | |
| Dendritic cells | 0.2 | Dermal fibroblast | 0.6 |
| none | | IFN gamma | |
| Dendritic cells | 0.3 | Dermal fibroblast | 6.7 |
| LPS | | IL-4 | |
| Dendritic cells | 0.7 | IBD Colitis 2 | 0.7 |
| anti-CD40 | | | |
| Monocytes rest | 0.0 | IBD Crohn's | 4.0 |
| Monocytes LPS | 0.0 | Colon | 86.5 |
| Macrophages rest | 0.0 | Lung | 3.6 |
| Macrophages LPS | 0.0 | Thymus | 100.0 |
| HUVEC none | 2.0 | Kidney | 7.7 |
| HUVEC starved | 5.7 | | |

CNS_neurodegeneration_v1.0 Summary: Ag3350 This panel does not show differential expression of the CG57853-01 gene in Alzheimer's disease. However, this expression profile confirms the presence of this gene in the brain. Please see Panel 1.4 for discussion of utility of this gene in the central nervous system.

General_screening_panel_v1.4 Summary: Ag3350—The CG157853-01 gene encodes a novel ileal sodium-bile acid cotransporter. Ileal sodium-dependent bile acid transporters play a role in the absorption of bile acids, regulating levels of bile acids in the large intestine and preventing accumulation of cytotoxic secondary bile acids in the colon (Ref. 1). The highest expression of this gene is seen in gastric cancer cell line NCI-N87 (CT=29.5). Significant expression of this gene is also associated with CNS, colon, renal, liver, lung, breast, ovarian and prostate cancers as well as melanomas. Thus, expression of this gene could be used as a diagnostic marker for the presence of these cancers. Furthermore, therapeutic inhibition using antibodies or small molecule drugs might be of use in the treatment of these cancers.

Among tissues with metabolic or endocrine function, this gene is expressed at low to moderate levels in pancreas, adipose, fetal skeletal muscle, heart, fetal liver and the gastrointestinal tract. Therefore, therapeutic modulation of the activity of this gene may prove useful in the treatment of endocrine/metabolically related diseases, such as intestinal bile acid malabsorption (IBAM), hypertriglyceridemia, obesity and diabetes.

Interestingly, this gene is expressed at much higher levels in fetal (CT=30.6) when compared to adult liver (CT=36.5). This observation suggests that expression of this gene can be used to distinguish fetal from adult liver. In addition, modulation of expression of this gene may be useful in the treatment of liver related disease such as liver cirrhosis, and cholestasis.

This gene is also widely expressed in tissues originating in the central nervous system. These tissues include the hippocampus, thalamus, and cerebellum. This transporter gene most likely plays a role in the uptake of nutrients. Blockade of this transporter may decrease the loss of neurons due to excitotoxicity during ischemic stroke. In addition, this gene may play a role in central nervous system disorders such as Alzheimer's disease, Parkinson's disease, epilepsy, multiple sclerosis, schizophrenia and depression.

References.

1. Small D M. (1997) Point mutations in the ileal bile salt transporter cause leaks in the enterohepatic circulation leading to severe chronic diarrhea and malabsorption. J Clin Invest 99(8):1807-8

2. Montagnani M, Love M W, Rossel P, Dawson P A, Qvist P. Absence of dysfunctional ileal sodium-bile acid cotransporter gene mutations in patients with adult-onset idiopathic bile acid malabsorption. Scand J Gastroenterol Octiber 2001;36(10):1077-80.

3. Wang W, Xue S, Ingles S A, Chen Q, Diep A T, Frankl H D, Stolz A, Haile R W. An association between genetic polymorphisms in the ileal sodium-dependent bile acid transporter gene and the risk of colorectal adenomas. Cancer Epidemiol Biomarkers Prev 2001 Sep;10(9):931-6.

4. Duane W C, Hartich L A, Bartman A E, Flo S B. Diminished gene expression of ileal apical sodium bile acid transporter explains impaired absorption of bile acid in patients with hypertriglyceridemia. J Lipid Res September 2000;41(9):1384-9.

Panel 1.3D Summary: Ag2838—Results using this primer pair are in very good agreement with the results obtained using Ag3350 on panel 1.4. Please see panel 1.4 for discussion.

Panel 4D Summary: Ag3350 Moderate to low expression of the CG57853-01 gene is seen in many samples on this panel, with the highest expression in thymus tissue (CT=29.3). This gene is expressed at low to moderate levels in a wide range of cell types of significance in the immune response in health and disease. These cells include members of the A-cell, B-cell, endothelial cell, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and ostearthritis.

Interestingly, high expression of this gene is detected in normal colon (CT=29.5), but expression of this gene is decreased in colon samples from patients with IBD colitis (CT=36.4) and Crohn's disease (CT=34.0). Therefore, therapeutic modulation of the activity of the transporter encoded by this gene may be useful in the treatment of inflammatory bowel disease.

In addition, expression of this gene is up regulated in PWM/PHA-L-stimulated PBMC (CTs=32-33.6), compared to resting PBMC cells (CT=37.6). Thus, expression of this gene may be used as a marker for activated PBMC cells.

### E. NOV5a AND NOV5b (CG57829-01 AND CC57829-05: A DISINTEGRIN AND METALLOPROTEINASE WITH THROMBOSPONDIN MOTIFS 1)

Expression of gene CG57829-01 and CG57829-05 was assessed using the primer-probe sets Ag3340 and Ag554, described in Tables EA and EB. Results of the RTQ-PCR runs are shown in Tables EC, ED, EE, EF, EG, EH, EI, EJ, EK, EL and EM. Please note that CG57829-05 represents a full-length physical clone.

TABLE EA
Probe Name Ag3340

| | | | | Start |
|---|---|---|---|---|
| Primers | Sequences | | Length | Positon |
| Forward | 5′-aatggcactggctacttctatg-3′ | (SEQ ID NO:224) | 22 | 1894 |
| Probe | TET-5′-acgctgtgctctcctgactccacct-3′-TAMRA | (SEQ ID NO: 225) | 25 | 1942 |
| Reverse | 5′-cacttgccttggacacaga-3′ | (SEQ ID NO:226) | 19 | 1970 |

TABLE EB
Probe Name Ag554

| | | | | Start |
|---|---|---|---|---|
| Primers | Sequences | | Length | Position |
| Forward | 5′-cctggcagaagaagctgaaca-3′ | (SEQ ID NO:227) | 21 | 905 |
| Probe | TET-5′-tgacaagcaccccgagtactgggac-3′-TAMRA | (SEQ ID NO:228) | 25 | 933 |
| Reverse | 5′-tgcctggtgaagaggatgg-3′ | (SEQ ID NO:229) | 19 | 962 |

TABLE EC
AI_comprehensive panel_v1.0

| | Rel. Exp.(%) | | Rel. Exp.(%) |
|---|---|---|---|
| | Ag3340, Run | | Ag3340, Run |
| Tissue Name | 211063355 | Tissue Name | 211063355 |
| 110967 COPD-F | 2.5 | 112427 Match | 1.3 |
| | | Control Psoriasis-F | |
| 110980 COPD-F | 1.3 | 112418 Psoriasis-M | 2.5 |
| 110968 COPD-M | 3.6 | 112723 Match | 0.0 |
| | | Control Psoriasis-M | |
| 110977 COPD-M | 2.4 | 112419 Psoriasis-M | 2.4 |
| 110989 | 6.2 | 112424 Match | 1.0 |
| Emphysema-F | | Control Psoriasis-M | |
| 110992 | 12.2 | 112420 Psoriasis-M | 11.3 |
| Emphysema-F | | | |
| 110993 | 5.0 | 112425 Match | 0.0 |
| Emphysema-F | | Control Psoriasis-M | |
| 110994 | 2.4 | 104689 (MF) OA | 6.2 |
| Emphysema-F | | Bone-Backus | |
| 110995 | 14.2 | 104690 (MF) Adj | 8.1 |
| Emphysema-F | | “Normal” Bone- | |
| | | Backus | |
| 110996 | 13.5 | 104691 (MF) OA | 12.5 |
| Emphysema-F | | Synovium-Backus | |
| 110997 | 0.0 | 104692 (BA) OA | 0.0 |
| Asthma-M | | Cartilage-Backus | |
| 111001 | 5.5 | 104694 (BA) OA | 6.7 |
| Asthma-M | | Bone-Backus | |
| 111002 | 4.1 | 104695 (BA) Adj | 8.0 |
| Asthma-M | | “Normal” Bone- | |
| | | Backus | |
| 111003 Atopic | 10.6 | 104696 (BA) OA | 25.2 |
| Asthma-F | | Synovium-Backus | |
| 111004 Atopic | 9.2 | 104700 (SS) OA | 1.9 |
| Asthma-F | | Bone-Backus | |
| 111005 Atopic | 6.6 | 104701 (SS) Adj | 11.9 |
| Asthma-F | | “Normal” Bone- | |
| | | Backus | |
| 111006 Atopic | 1.5 | 104702 (SS) OA | 32.3 |
| Asthma-F | | Synovium-Backus | |
| 111417 | 3.3 | 117093 OA | 6.9 |
| Allergy-M | | Cartilage Rep7 | |
| 112347 | 0.2 | 112672 OA Bone5 | 1.5 |
| Allergy-M | | | |
| 112349 Normal | 0.1 | 112673 OA | 0.9 |
| Lung-F | | Synovium5 | |
| 112357 Normal | 4.1 | 112674 OA | 0.8 |
| Lung-F | | Synovial | |
| | | Fluid cells5 | |
| 112354 Normal | 1.7 | 117100 OA | 1.9 |
| Lung-M | | Cartilage Rep14 | |
| 112374 Crohns-F | 5.2 | 112756 OA Bone9 | 2.7 |
| 112389 Match | 1.8 | 112757 OA | 2.1 |
| Control Crohns-F | | Synovium9 | |
| 112375 Crohns-F | 3.6 | 112758 OA | 4.6 |
| | | Synovial | |
| | | Fluid Cells9 | |
| 112732 Match | 0.4 | 117125 RA | 7.6 |
| Control Crohns-F | | Cartilage Rep2 | |
| 112725 Crohns-M | 0.1 | 113492 Bone2 RA | 2.5 |
| 112387 Match | 2.7 | 113493 Synovium2 | 1.1 |
| Control | | RA | |
| Crohns-M | | | |
| 112378 Crohns-M | 0.1 | 113494 Syn Fluid | 0.9 |
| | | Cells RA | |
| 112390 Match | 1.7 | 113499 Cartilage4 | 2.9 |
| Control | | RA | |
| Crohns-M | | | |
| 112726 Crohns-M | 10.2 | 113500 Bone4 RA | 1.6 |
| 112731 Match | 1.5 | 113501 Synovium4 | 2.0 |
| Control | | RA | |
| Crohns-M | | | |
| 112380 Ulcer | 0.6 | 113502 Syn Fluid | 0.4 |
| Col-F | | Cells4 RA | |
| 112734 Match | 0.6 | 113495 Cartilage3 | 1.7 |
| Control Ulcer | | RA | |
| Col-F | | | |
| 112384 Ulcer | 23.7 | 113496 Bone3 RA | 3.1 |
| Col-F | | | |
| 112737 Match | 2.9 | 113497 Synovium3 | 1.2 |
| Control Ulcer | | RA | |
| Col-F | | | |
| 112386 Ulcer | 2.6 | 113498 Syn Fluid | 2.6 |
| Col-F | | Cells3 RA | |
| 112738 Match | 4.9 | 117106 Normal | 7.1 |
| Control Ulcer | | Cartilage Rep20 | |
| Col-F | | | |
| 112381 Ulcer | 0.1 | 113663 Bone3 | 0.2 |
| Col-M | | Normal | |
| 112735 Match | 1.2 | 113664 Synovium3 | 0.1 |
| Control Ulcer | | Normal | |
| Col-M | | | |
| 112382 Ulcer | 1.5 | 113665 Syn Fluid | 0.1 |
| Col-M | | Cells3 Normal | |
| 112394 Match | 0.4 | 117107 Normal | 1.0 |
| Control Ulcer | | Cartilage Rep22 | |
| Col-M | | | |
| 112383 Ulcer | 100.0 | 113667 Bone4 | 1.1 |
| Col-M | | Normal | |
| 112736 Match | 0.8 | 113668 Synovium4 | 1.6 |
| Control Ulcer | | Normal | |
| Col-M | | | |
| 112423 | 0.0 | 113669 Syn Fluid | 2.8 |
| Psoriasis-F | | Cells4 Normal | |

TABLE ED
CNS_neurodegeneration_v1.0

| | Rel. Exp.(%) | | Rel. Exp.(%) |
|---|---|---|---|
| | Ag3340, Run | | Ag3340, Run |
| Tissue Name | 210139046 | Tissue Name | 210139046 |
| AD 1 Hippo | 0.0 | Control (Path) 3 | 22.5 |
| | | Temporal Ctx | |
| AD 2 Hippo | 51.4 | Control (Path) 4 | 32.1 |
| | | Temporal Ctx | |
| AD 3 Hippo | 12.7 | AD 1 Occipital Ctx | 17.8 |
| AD 4 Hippo | 14.3 | AD 2 Occipital Ctx | 0.0 |
| | | (Missing) | |
| AD 5 hippo | 12.9 | AD 3 Occipital Ctx | 0.0 |
| AD 6 Hippo | 40.3 | AD 4 Occipital Ctx | 23.8 |
| Control 2 Hippo | 36.3 | AD 5 Occipital Ctx | 9.0 |
| Conirol 4 Hippo | 40.3 | AD 6 Occipital Ctx | 100.0 |
| Control (Path) 3 | 4.3 | Control 1 Occipital | 15.1 |
| Hippo | | Ctx | |
| AD 1 Temporal | 0.0 | Control 2 Occipital | 25.0 |
| Ctx | | Ctx | |
| AD 2 Temporal | 69.7 | Control 3 Occipital | 38.4 |
| Ctx | | Ctx | |
| AD 3 Temporal | 16.4 | Control 4 Occipital | 8.1 |
| Ctx | | Ctx | |
| AD 4 Temporal | 47.6 | Control (Path) 1 | 35.6 |
| Ctx | | Occipital Ctx | |
| AD 5 Inf | 85.9 | Control (Path) 2 | 0.0 |
| Temporal Ctx | | Occipital Ctx | |
| AD 5 Sup | 44.4 | Control (Path) 3 | 0.0 |
| Temporal Ctx | | Occipital Ctx | |
| AD 6 Inf | 79.6 | Control (Path) 4 | 11.4 |
| Temporal Ctx | | Occipital Ctx | |
| AD 6 Sup | 57.0 | Control 1 Parietal | 29.3 |
| Temporal Ctx | | Ctx | |
| Control 1 | 9.3 | Control 2 Parietal | 53.6 |
| Temporal Ctx | | Ctx | |
| Control 2 | 32.3 | Control 3 Parietal | 37.4 |
| Temporal Ctx | | Ctx | |
| Control 3 | 19.6 | Control (Path) 1 | 18.7 |
| Temporal Ctx | | Parietal Ctx | |
| Control 4 | 13.2 | Control (Path) 2 | 29.5 |
| Temporal Ctx | | Parietal Ctx | |
| Control (Path) 1 | 77.4 | Control (Path) 3 | 7.0 |
| Temporal Ctx | | Parietal Ctx | |
| Control (Path) 2 | 39.0 | Control (Path) 4 | 38.7 |
| Temporal Ctx | | Parietal Ctx | |

TABLE EE
General_screening_panel_v1.4

| | Rel. Exp.(%) | Rel. Exp.(%) | | Rel. Exp.(%) | Rel. Exp.(%) |
|---|---|---|---|---|---|
| | Ag3340, Run | Ag3340, Run | | Ag3340, Run | Ag3340, Run |
| Tissue Name | 213333210 | 217049357 | Tissue Name | 213333210 | 217049357 |
| Adipose | 9.2 | 9.1 | Renal ca. TK-10 | 3.5 | 4.5 |
| Melanoma* | 0.0 | 0.0 | Bladder | 1.7 | 2.3 |
| Hs688(A).T | | | | | |
| Melanoma* | 1.7 | 1.9 | Gastric ca. (liver | 3.3 | 4.0 |
| Hs688(B).T | | | met.) NCI-N87 | | |
| Melanoma* | 0.0 | 0.0 | Gastric ca. KATO | 5.9 | 7.7 |
| M14 | | | III | | |
| Melanoma* | 0.0 | 0.0 | Colon ca. SW- | 3.9 | 4.6 |
| LOXIMVI | | | 948 | | |
| Melanoma* | 0.2 | 0.4 | Colon ca. SW480 | 21.0 | 24.3 |
| SK-MEL-5 | | | | | |
| Squamous cell | 0.1 | 0.2 | Colon ca.* | 1.8 | 1.6 |
| carcinoma | | | (SW480 met) | | |
| SCC-4 | | | SW620 | | |
| Testis Pool | 2.1 | 1.1 | Colon ca. HT29 | 0.0 | 0.0 |
| Prostate ca.* | 4.1 | 3.6 | Colon ca. HCT- | 1.1 | 1.0 |
| (bone met) | | | 116 | | |
| PC-3 | | | | | |
| Prostate Pool | 0.6 | 0.4 | Colon ca. CaCo-2 | 1.9 | 2.2 |
| Placenta | 6.6 | 6.7 | Colon cancer | 2.8 | 2.6 |
| | | | tissue | | |
| Uterus Pool | 0.5 | 0.5 | Colon ca. | 0.1 | 0.2 |
| | | | SW1116 | | |
| Ovarian ca. | 50.0 | 37.1 | Colon ca. Colo- | 0.1 | 0.2 |
| OVCAR-3 | | | 205 | | |
| Ovarian ca. | 43.2 | 29.9 | Colon ca. SW-48 | 0.0 | 0.0 |
| SK-OV-3 | | | | | |
| Ovarian ca. | 31.4 | 36.6 | Colon Pool | 2.5 | 3.1 |
| OVCAR-4 | | | | | |
| Ovarian ca. | 28.1 | 33.0 | Small Intestine | 0.4 | 0.8 |
| OVCAR-5 | | | Pool | | |
| Ovarian ca. | 8.5 | 8.5 | Stomach Pool | 1.8 | 1.5 |
| IGROV-1 | | | | | |
| Ovarian ca. | 3.6 | 5.7 | Bone Marrow | 0.7 | 0.7 |
| OVCAR-8 | | | Pool | | |
| Ovary | 1.1 | 1.2 | Fetal Heart | 6.7 | 7.6 |
| Breast ca. | 0.8 | 0.6 | Heart Pool | 2.5 | 2.8 |
| MCF-7 | | | | | |
| Breast ca. | 100.0 | 100.0 | Lymph Node | 4.0 | 5.0 |
| MDA-MB-231 | | | Pool | | |
| Breast ca. BT | 46.0 | 43.5 | Fetal Skeletal | 2.4 | 1.9 |
| 549 | | | Muscle | | |
| Breast ca. | 78.5 | 72.2 | Skeletal Muscle | 1.5 | 1.2 |
| T47D | | | Pool | | |
| Breast ca. | 0.0 | 0.0 | Spleen Pool | 0.9 | 1.0 |
| MDA-N | | | | | |
| Breast Pool | 2.1 | 1.7 | Thymus Pool | 2.2 | 2.1 |
| Trachea | 1.0 | 1.7 | CNS cancer | 0.0 | 0.0 |
| | | | (glio/astro) U87- | | |
| | | | MG | | |
| Lung | 0.4 | 0.3 | CNS cancer | 46.0 | 52.5 |
| | | | (glio/astro) U- | | |
| | | | 118-MG | | |
| Fetal Lung | 14.4 | 14.7 | CNS cancer | 1.4 | 1.6 |
| | | | (neuro;met) SK- | | |
| | | | N-AS | | |
| Lung ca. NCI- | 0.2 | 0.0 | CNS cancer | 3.0 | 3.1 |
| N417 | | | (astro) SF-539 | | |
| Lung ca. LX-1 | 4.5 | 4.4 | CNS cancer | 13.2 | 27.9 |
| | | | (astro) SNB-75 | | |
| Lung ca. NCI- | 0.0 | 0.0 | CNS cancer (glio) | 7.2 | 9.1 |
| H146 | | | SNB-19 | | |
| Lung ca. SHP- | 17.4 | 17.7 | CNS cancer (glio) | 1.8 | 1.7 |
| 77 | | | SF-295 | | |
| Lung ca. A549 | 8.4 | 6.6 | Brain (Amygdala) | 0.2 | 0.1 |
| | | | Pool | | |
| Lung ca. NCI- | 0.0 | 0.0 | Brain | 0.2 | 0.3 |
| H526 | | | (cerebellum) | | |
| Lung ca. NCI- | 0.4 | 0.3 | Brain (fetal) | 3.0 | 1.8 |
| H23 | | | | | |
| Lung ca. NCI- | 0.1 | 0.1 | Brain | 0.4 | 0.4 |
| H460 | | | (Hippocampus) | | |
| | | | Pool | | |
| Lung ca. | 0.7 | 0.5 | Cerebral Cortex | 0.5 | 0.2 |
| HOP-62 | | | Pool | | |
| Lung ca. NCI- | 0.2 | 0.1 | Brain (Substantia | 0.6 | 0.2 |
| H522 | | | nigra) Pool | | |
| Liver | 0.0 | 0.0 | Brain (Thalamus) | 0.2 | 0.2 |
| | | | Pool | | |
| Fetal Liver | 0.8 | 0.8 | Brain (whole) | 0.6 | 0.7 |
| Liver ca. | 0.0 | 0.0 | Spinal Cord Pool | 1.1 | 1.0 |
| HepG2 | | | | | |
| Kidney Pool | 2.6 | 2.5 | Adrenal Gland | 1.2 | 0.9 |
| Fetal Kidney | 12.4 | 15.1 | Pituitary gland | 0.3 | 0.4 |
| | | | Pool | | |
| Renal ca. 786-0 | 15.8 | 18.7 | Salivary Gland | 0.3 | 0.2 |
| Renal ca. | 3.5 | 4.1 | Thyroid (female) | 2.9 | 2.6 |
| A498 | | | | | |
| Renal ca. | 53.6 | 48.0 | Pancreatic ca. | 1.7 | 1.6 |
| ACHN | | | CAPAN2 | | |
| Renal ca. UO- | 13.1 | 13.0 | Pancreas Pool | 7.1 | 5.3 |
| 31 | | | | | |

TABLE EF
Panel 1.1

| | Rel. Exp.(%) | | Rel. Exp.(%) |
|---|---|---|---|
| | Ag554, Run | | Ag554, Run |
| Tissue Name | 109665128 | Tissue Name | 109665128 |
| Adrenal gland | 0.1 | Renal ca. UO-31 | 6.2 |
| Bladder | 4.2 | Renal ca. RXF 393 | 0.2 |
| Brain (amygdala) | 0.0 | Liver | 0.3 |
| Brain | 0.0 | Liver (fetal) | 0.7 |
| (cerebellum) | | | |
| Brain | 0.1 | Liver ca. | 0.0 |
| (hippocampus) | | (hepatoblast) HepG2 | |
| Brain (substantia | 0.0 | Lung | 4.3 |
| nigra) | | | |
| Brain (thalamus) | 0.0 | Lung (fetal) | 13.2 |
| Cerebral Cortex | 0.0 | Lung ca. (non-s. | 0.3 |
| | | cell) HOP-62 | |
| Brain (fetal) | 0.5 | Lung ca. (large | 0.0 |
| | | cell) NCI-H460 | |
| Brain (whole) | 0.0 | Lung ca. (non-s.) | 0.0 |
| | | cell) NCI-H23 | |
| glio/astro | 34.9 | Lung ca. (non-s.cl) | 0.0 |
| U-118-MG | | NCI-H522 | |
| astrocytoma | 1.7 | Lung ca. (non-sm. | 0.1 |
| SF-539 | | cell) A549 | |
| astrocytoma | 13.3 | Lung ca. (s.cell | 9.9 |
| SNB-75 | | var.) SHP-77 | |
| astrocytoma | 1.2 | Lung ca. (small | 3.2 |
| SW1783 | | cell) LX-1 | |
| glioma U251 | 24.0 | Lung ca. (small | 0.1 |
| | | cell) NCI-H69 | |
| glioma SF-295 | 0.0 | Lung ca. (squam.) | 0.0 |
| | | SW 900 | |
| glioma SNB-19 | 27.0 | Lung ca. (squam.) | 0.6 |
| | | NCI-H596 | |
| glio/astro | 0.0 | Lymph node | 1.9 |
| U87-MG | | | |
| neuro*; met | 0.1 | Spleen | 0.0 |
| SK-N-AS | | | |
| Mammary gland | 34.9 | Thymus | 0.1 |
| Breast ca. BT-549 | 8.4 | Ovary | 13.0 |
| Breast ca. | 0.0 | Ovarian ca. | 4.6 |
| MDA-N | | IGROV-1 | |
| Breast ca.* | 95.3 | Ovarian ca. | 100.0 |
| (pl.ef) T47D | | OVCAR-3 | |
| Breast ca.* | 0.0 | Ovarian ca. | 88.3 |
| (pl.ef) MCF-7 | | OVCAR-4 | |
| Breast ca.* | 50.0 | Ovarian ca. | 44.4 |
| (pl.ef) | | OVCAR-5 | |
| MDA-MB-231 | | | |
| Small intestine | 0.5 | Ovarian ca. | 0.0 |
| | | OVCAR-8 | |
| Colorectal | 1.1 | Ovarian ca.* | 57.4 |
| | | (ascites) SK-OV-3 | |
| Colon ca. HT29 | 0.0 | Pancreas | 0.0 |
| Colon ca. | 0.0 | Pancreatic ca. | 0.0 |
| CaCo-2 | | CAPAN2 | |
| Colon ca. | 0.0 | Pituitary gland | 0.8 |
| HCT-15 | | | |
| Colon ca. | 0.0 | Placenta | 21.6 |
| HCT-116 | | | |
| Colon ca. | 0.0 | Prostate | 0.0 |
| HCC-2998 | | | |
| Colon ca. SW480 | 7.5 | Prostate ca.* (bone | 3.0 |
| | | met) PC-3 | |
| Colon ca.* | 0.4 | Salivary gland | 0.0 |
| SW620 (SW480 | | | |
| met) | | | |
| Stomach | 5.4 | Trachea | 0.6 |
| Gastric ca. (liver | 1.1 | Spinal cord | 1.4 |
| met) NCI-N87 | | | |
| Heart | 54.7 | Testis | 0.0 |
| Skeletal muscle | 69.7 | Thyroid | 6.5 |
| (Fetal) | | | |
| Skeletal muscle | 0.9 | Uterus | 1.1 |
| Endothelial cells | 0.0 | Melanoma M14 | 0.0 |
| Heart (Fetal) | 55.9 | Melanoma LOX | 0.0 |
| | | IMVI | |
| Kidney | 35.6 | Melanoma | 0.0 |
| | | UACC-62 | |
| Kidney (fetal) | 59.9 | Melanoma | 0.0 |
| | | SK-MEL-28 | |
| Renal ca. 786-0 | 16.6 | Melanoma* (met) | 0.0 |
| | | SK-MEL-5 | |
| Renal ca. A498 | 7.7 | Melanoma | 0.0 |
| | | Hs688(A).T | |
| Renal ca. ACHN | 94.6 | Melanoma* (met) | 0.0 |
| | | Hs688(B).T | |
| Renal ca. TK-10 | 5.8 | | |

TABLE EG
Panel 1.2

| | Rel. Exp.(%) | Rel. Exp.(%) | | Rel. Exp.(%) | Rel. Exp.(%) |
|---|---|---|---|---|---|
| | Ag554, Run | Ag554, Run | | Ag554, Run | Ag554, Run |
| Tissue Name | 121754027 | 124088899 | Tissue Name | 121754027 | 124088899 |
| Endothelial cells | 0.0 | 0.0 | Renal ca. 786-0 | 5.8 | 0.0 |
| Heart (Fetal) | 69.3 | 56.6 | Renal ca. A498 | 2.3 | 0.0 |
| Pancreas | 0.0 | 0.0 | Renal ca. RXF | 0.0 | 0.0 |
| | | | 393 | | |
| Pancreatic ca. | 0.0 | 0.0 | Renal ca. | 44.1 | 9.6 |
| CAPAN 2 | | | ACHN | | |
| Adrenal Gland | 0.1 | 0.0 | Renal ca. UO- | 0.5 | 0.0 |
| | | | 31 | | |
| Thyroid | 0.2 | 0.0 | Renal ca. TK- | 0.1 | 0.0 |
| | | | 10 | | |
| Salivary gland | 0.0 | 0.0 | Liver | 0.1 | 0.0 |
| Pituitary gland | 0.0 | 0.0 | Liver (fetal) | 0.0 | 0.0 |
| Brain (fetal) | 0.0 | 0.0 | Liver ca. | 0.0 | 0.0 |
| | | | (hepatoblast) | | |
| | | | HepG2 | | |
| Brain (whole) | 0.0 | 0.0 | Lung | 3.2 | 0.0 |
| Brain (amygdala) | 0.0 | 0.0 | Lung (fetal) | 6.0 | 0.4 |
| Brain | 0.0 | 0.0 | Lung ca. (small | 0.0 | 0.0 |
| (cerebellum) | | | cell) LX-1 | | |
| Brain | 0.0 | 0.0 | Lung ca. (small | 0.0 | 0.0 |
| (hippocampus) | | | cell) NCI-H69 | | |
| Brain (thalamus) | 0.0 | 0.0 | Lung ca. (s.cell | 4.2 | 0.0 |
| | | | var.) SHP-77 | | |
| Cerebral Cortex | 0.0 | 0.0 | Lung ca. (large | 0.0 | 0.0 |
| | | | cell)NCI-H460 | | |
| Spinal cord | 0.0 | 0.0 | Lung ca. (non- | 0.0 | 0.0 |
| | | | sm. cell) A549 | | |
| glio/astro U87- | 0.0 | 0.0 | Lung ca. (non- | 0.0 | 0.0 |
| MG | | | s.cell) NCI- | | |
| | | | H23 | | |
| glio/astro U-118- | 18.0 | 0.2 | Lung ca. (non- | 0.0 | 0.0 |
| MG | | | s.cell) HOP-62 | | |
| astrocytoma | 0.0 | 0.0 | Lung ca. (non- | 0.0 | 0.0 |
| SW1783 | | | s.cl) NCI-H522 | | |
| neuro*; met SK- | 0.0 | 0.0 | Lung ca. | 0.0 | 0.0 |
| N-AS | | | (squam.) SW | | |
| | | | 900 | | |
| astrocytoma SF- | 0.0 | 0.0 | Lung ca. | 0.0 | 0.0 |
| 539 | | | (squam.) NCI- | | |
| | | | H596 | | |
| astrocytoma | 1.2 | 0.0 | Mammary | 24.1 | 18.0 |
| SNB-75 | | | gland | | |
| glioma SNB-19 | 14.7 | 2.8 | Breast ca.* | 0.0 | 0.0 |
| | | | (pl.ef) MCF-7 | | |
| glioma U251 | 6.2 | 0.0 | Breast ca.* | 31.0 | 12.7 |
| | | | (pl.ef) MDA- | | |
| | | | MB-231 | | |
| glioma SF-295 | 0.0 | 0.0 | Breast ca.* (pl. | 39.8 | 17.6 |
| | | | ef) T47D | | |
| Heart | 79.0 | 6.4 | Breast ca. BT- | 12.2 | 0.6 |
| | | | 549 | | |
| Skeletal Muscle | 0.0 | 0.0 | Breast ca. | 0.0 | 0.0 |
| | | | MDA-N | | |
| Bone marrow | 0.0 | 0.0 | Ovary | 7.4 | 0.0 |
| Thymus | 0.0 | 0.0 | Ovarian ca. | 37.1 | 24.8 |
| | | | OVCAR-3 | | |
| Spleen | 0.0 | 0.0 | Ovarian ca. | 54.3 | 16.7 |
| | | | OVCAR-4 | | |
| Lymph node | 0.0 | 0.0 | Ovarian ca. | 16.6 | 2.9 |
| | | | OVCAR-5 | | |
| Colorectal Tissue | 1.5 | 0.0 | Ovarian ca. | 0.0 | 0.0 |
| | | | OVCAR-8 | | |
| Stomach | 5.3 | 0.0 | Ovarian ca. | 2.1 | 0.0 |
| | | | IGROV-1 | | |
| Small intestine | 0.0 | 0.0 | Ovarian ca. | 21.2 | 4.7 |
| | | | (ascites) SK- | | |
| | | | OV-3 | | |
| Colon ca. | 0.0 | 0.0 | Uterus | 0.1 | 0.0 |
| SW480 | | | | | |
| Colon ca.* | 0.0 | 0.0 | Placenta | 38.7 | 2.2 |
| SW620 (SW480 | | | | | |
| met) | | | | | |
| Colon ca. HT29 | 0.0 | 0.0 | Prostate | 0.0 | 0.0 |
| Colon ca. HCT- | 0.0 | 0.0 | Prostate ca.* | 5.6 | 0.0 |
| 116 | | | (bone met) | | |
| | | | PC-3 | | |
| Colon ca. CaCo-2 | 0.0 | 0.0 | Testis | 0.0 | 0.0 |
| Colon ca. Tissue | 0.1 | 0.0 | Melanoma | 0.0 | 0.0 |
| (ODO3866) | | | Hs688(A).T | | |
| Colon ca. HCC- | 0.0 | 0.0 | Melanoma* | 0.0 | 0.0 |
| 2998 | | | (met) | | |
| | | | Hs688(B).T | | |
| Gastric ca.* | 0.7 | 0.0 | Melanoma | 0.0 | 0.0 |
| (liver met) NCI- | | | UACC-62 | | |
| N87 | | | | | |
| Bladder | 1.9 | 0.0 | Melanoma | 0.0 | 0.0 |
| | | | M14 | | |
| Trachea | 0.2 | 0.0 | Melanoma | 0.0 | 0.0 |
| | | | LOX IMVI | | |
| Kidney | 11.0 | 0.6 | Melanoma* | 0.0 | 0.0 |
| | | | (met) SK- | | |
| | | | MEL-5 | | |
| Kidney (fetal) | 100.0 | 100.0 | | | |

TABLE EH
Panel 1.3D

| | Rel. Exp.(%) Ag554, | | Rel. Exp.(%) Ag554, |
|---|---|---|---|
| Tissue Name | Run 165702013 | Tissue Name | Run 165702013 |
| Liver adenocarcinoma | 0.2 | Kidney (fetal) | 15.7 |
| Pancreas | 0.1 | Renal ca. 786-0 | 9.0 |
| Pancreatic ca. CAPAN2 | 0.6 | Renal ca. A498 | 7.3 |
| Adrenal gland | 0.8 | Renal ca. RXF 393 | 4.1 |
| Thyroid | 5.0 | Renal ca. ACHN | 42.9 |
| Salivary gland | 0.7 | Renal ca. UO-31 | 33.9 |
| Pituitary gland | 0.8 | Renal ca. TK-10 | 2.2 |
| Brain (fetal) | 0.3 | Liver | 0.3 |
| Brain (whole) | 0.2 | Liver (fetal) | 1.3 |
| Brain (amygdala) | 1.2 | Liver ca. | 0.0 |
| | | (hepatoblast) HepG2 | |
| Brain (cerebellum) | 0.2 | Lung | 5.4 |
| Brain (hippocampus) | 1.2 | Lung (fetal) | 10.7 |
| Brain (substantia nigra) | 0.5 | Lung ca. (small cell) | 2.7 |
| | | LX-1 | |
| Brain (thalamus) | 0.3 | Lung ca. (small cell) | 1.1 |
| | | NCI-H69 | |
| Cerebral Cortex | 0.7 | Lung ca. (s.cell var.) | 19.3 |
| | | SHP-77 | |
| Spinal cord | 1.9 | Lung ca. (large | 0.7 |
| | | cell) NCI-H460 | |
| glio/astro U87-MG | 0.0 | Lung ca. (non-sm. | 1 .2 |
| | | cell) A549 | |
| glio/astro U-118-MG | 75.8 | Lung ca. (non-s.cell) | 0.0 |
| | | NCI-H23 | |
| astrocytoma SW1783 | 5.1 | Lung ca. (non-s.cell) | 1.0 |
| | | HOP-62 | |
| neuro*; met SK-N-AS | 0.7 | Lung ca. (non-s.cl) | 0.0 |
| | | NCI-H522 | |
| astrocytoma SF-539 | 3.3 | Lung ca. (squam.) | 0.8 |
| | | SW 900 | |
| astrocytoma SNB-75 | 13.5 | Lung ca. (squam.) | 1.3 |
| | | NCI-H596 | |
| glioma SNB-19 | 24.0 | Mammary gland | 15.3 |
| glioma U251 | 48.0 | Breast ca.* (pl.ef) | 0.9 |
| | | MCF-7 | |
| glioma SF-295 | 0.3 | Breast ca.* (pl.ef) | 100.0 |
| | | MDA-MB-231 | |
| Heart (fetal) | 22.2 | Breast ca.* (pl.ef) | 34.6 |
| | | T47D | |
| Heart | 10.7 | Breast ca. BT-549 | 21.3 |
| Skeletal muscle (fetal) | 22.1 | Breast ca. MDA-N | 0.0 |
| Skeletal muscle | 2.4 | Ovary | 3.1 |
| Bone marrow | 0.0 | Ovarian ca. OVCAR-3 | 31.9 |
| Thymus | 0.8 | Ovarian ca. OVCAR-4 | 43.2 |
| Spleen | 1.2 | Ovarian ca. OVCAR-5 | 19.3 |
| Lymph node | 4.3 | Ovarian ca. OVCAR-8 | 0.0 |
| Colorectal | 3.0 | Ovarian ca. IGROV-1 | 2.2 |
| Stomach | 3.0 | Ovarian ca.* (ascites) | 22.7 |
| | | SK-OV-3 | |
| Small intestine | 2.9 | Uterus | 4.4 |
| Colon ca. SW480 | 5.0 | Placenta | 6.7 |
| Colon ca.* | 0.5 | Prostate | 0.9 |
| SW620(SW480 met) | | | |
| Colon ca. HT29 | 0.0 | Prostate ca.* (bone | 2.2 |
| | | met) PC-3 | |
| Colon ca. HCT-116 | 0.0 | Testis | 1.0 |
| Colon ca. CaCo-2 | 0.5 | Melanoma | 0.0 |
| | | Hs688(A).T | |
| Colon ca. | 2.3 | Melanoma* (met) | 1.4 |
| tissue (ODO3866) | | Hs688(B).T | |
| Colon ca. HCC-2998 | 0.0 | Melanoma UACC-62 | 1.0 |
| Gastric ca.* (liver met) | 3.5 | Melanoma M14 | 0.0 |
| NCI-N87 | | | |
| Bladder | 1.2 | Melanoma LOX | 0.0 |
| | | IMVI | |
| Trachea | 1.7 | Melanoma* (met) | 0.3 |
| | | SK-MEL-5 | |
| Kidney | 7.9 | Adipose | 23.2 |

TABLE EI
Panel 2.1

| | Rel. Exp.(%) Ag3340, | | Rel. Exp.(%) Ag3340, |
|---|---|---|---|
| Tissue Name | Run 170686167 | Tissue Name | Run 170686167 |
| Normal Colon | 6.0 | Kidney Cancer | 7.0 |
| | | 9010320 | |
| Colon cancer (OD06064) | 0.9 | Kidney margin | 14.7 |
| | | 9010321 | |
| Colon cancer margin | 1.4 | Kidney Cancer | 4.2 |
| (OD06064) | | 8120607 | |
| Colon cancer (OD06159) | 0.3 | Kidney margin | 7.8 |
| | | 8120608 | |
| Colon cancer margin | 0.3 | Normal Uterus | 11.1 |
| (OD06159) | | | |
| Colon cancer (OD06298- | 2.6 | Uterus Cancer | 6.1 |
| 08) | | | |
| Colon cancer margin | 1.4 | Normal Thyroid | 1.4 |
| (OD06298-018) | | | |
| Colon Cancer Gr.2 ascend | 3.0 | Thyroid Cancer | 18.2 |
| colon (ODO3921) | | | |
| Colon Cancer margin | 2.1 | Thyroid Cancer | 0.6 |
| (ODO3921) | | A302152 | |
| Colon cancer metastasis | 2.5 | Thyroid margin | 4.4 |
| (OD06104) | | A302153 | |
| Lung margin (OD06104) | 1.6 | Normal Breast | 22.1 |
| Colon mets to lung | 0.0 | Breast Cancer | 10.4 |
| (OD04451-01) | | | |
| Lung margin (OD04451- | 3.5 | Breast Cancer | 6.7 |
| 02) | | | |
| Normal Prostate | 0.8 | Breast Cancer | 24.8 |
| | | (OD04590-01) | |
| Prostate Cancer | 0.4 | Breast Cancer Mets | 29.3 |
| (OD04410) | | (OD04590-03) | |
| Prostate margin | 1.9 | Breast Cancer | 100.0 |
| (OD04410) | | Metastasis | |
| Normal Lung | 14.4 | Breast Cancer | 3.4 |
| Invasive poor diff. lung | 0.3 | Breast Cancer | 14.4 |
| adeno 1 (ODO4945-01) | | 9100266 | |
| Lung margin (ODO4945- | 11.1 | Breast margin | 6.8 |
| 03) | | 9100265 | |
| Lung Malignant Cancer | 2.6 | Breast Cancer | 4.7 |
| (OD03126) | | A209073 | |
| Lung margin (OD03126) | 1.2 | Breast margin | 16.8 |
| | | A2090734 | |
| Lung Cancer (OD05014A) | 1.0 | Normal Liver | 0.9 |
| Lung margin (OD05014B) | 4.2 | Liver Cancer 1026 | 3.0 |
| Lung Cancer (OD04237- | 1.8 | Liver Cancer 1025 | 0.4 |
| 01) | | | |
| Lung margin (OD04237- | 5.1 | Liver Cancer 6004-T | 0.1 |
| 02) | | | |
| Ocular Mel Met to Liver | 0.0 | Liver Tissue 6004-N | 0.0 |
| (ODO4310) | | | |
| Liver margin (ODO4310) | 0.7 | Liver Cancer 6005-T | 5.9 |
| Melanoma Mets to Lung | 1.0 | Liver Tissue 6005-N | 3.7 |
| (OD04321) | | | |
| Lung margin (OD04321) | 6.4 | Liver Cancer | 0.3 |
| Normal Kidney | 20.9 | Normal Bladder | 2.2 |
| Kidney Ca, Nuclear grade | 23.3 | Bladder Cancer | 1.7 |
| 2 (OD04338) | | | |
| Kidney margin (OD04338) | 1.6 | Bladder Cancer | 0.9 |
| Kidney Ca Nuclear grade | 4.5 | Normal Ovary | 5.6 |
| 1/2 (OD04339) | | | |
| Kidney margin (OD04339) | 5.0 | Ovarian Cancer | 4.9 |
| Kidney Ca, Clear cell type | 1.6 | Ovarian cancer | 2.7 |
| (OD04340) | | (OD06145) | |
| Kidney margin (OD04340) | 10.4 | Ovarian cancer | 3.1 |
| | | margin (OD06145) | |
| Kidney Ca, Nuclear grade | 0.4 | Normal Stomach | 24.8 |
| 3 (OD04348) | | | |
| Kidney margin (OD04348) | 8.5 | Gastric Cancer | 0.0 |
| | | 9060397 | |
| Kidney Cancer (OD04450- | 0.5 | Stomach margin | 16.5 |
| 01) | | 9060396 | |
| Kidney margin (OD04450- | 12.7 | Gastric Cancer | 4.9 |
| 03) | | 9060395 | |
| Kidney Cancer 8120613 | 0.0 | Stomach margin | 11.3 |
| | | 9060394 | |
| Kidney margin 8120614 | 6.2 | Gastric Cancer | 2.1 |
| | | 064005 | |

TABLE EJ
Panel 2D

| | Rel. Exp.(%) | Rel. Exp.(%) | | Rel. Exp.(%) | Rel. Exp.(%) |
|---|---|---|---|---|---|
| | Ag554, Run | Ag554, Run | | Ag554, Run | Ag554, Run |
| Tissue Name | 145968942 | 145968960 | Tissue Name | 145968942 | 145968960 |
| Normal Colon | 12.9 | 15.4 | Kidney Margin | 12.2 | 14.3 |
| | | | 8120608 | | |
| CC Well to Mod | 1.9 | 1.9 | Kidney Cancer | 0.2 | 0.2 |
| Diff (ODO3866) | | | 8120613 | | |
| CC Margin | 3.0 | 3.8 | Kidney Margin | 8.7 | 16.2 |
| (ODO3866) | | | 8120614 | | |
| CC Gr.2 | 1.4 | 1.8 | Kidney Cancer | 17.1 | 21.3 |
| rectosigmoid | | | 9010320 | | |
| (ODO3868) | | | | | |
| CC Margin | 1.2 | 1.4 | Kidney Margin | 16.6 | 18.9 |
| (ODO3868) | | | 9010321 | | |
| CC Mod Diff | 0.5 | 0.5 | Normal Uterus | 2.9 | 4.2 |
| (ODO3920) | | | | | |
| CC Margin | 0.8 | 1.5 | Uterus Cancer | 5.3 | 4.7 |
| (ODO3920) | | | 064011 | | |
| CC Gr.2 ascend | 7.1 | 9.9 | Normal | 5.6 | 7.0 |
| colon | | | Thyroid | | |
| (ODO3921) | | | | | |
| CC Margin | 1.2 | 1.9 | Thyroid | 20.2 | 28.7 |
| (ODO3921) | | | Cancer 064010 | | |
| CC from Partial | 1.6 | 2.7 | Thyroid | 1.4 | 1.4 |
| Hepatectomy | | | Cancer | | |
| (ODO4309) Mets | | | A302152 | | |
| Liver Margin | 0.7 | 1.0 | Thyroid | 7.3 | 9.0 |
| (ODO4309) | | | Margin | | |
| | | | A302153 | | |
| Colon mets to | 3.1 | 5.8 | Normal Breast | 23.0 | 29.9 |
| lung (OD04451- | | | | | |
| 01) | | | | | |
| Lung Margin | 2.7 | 1.8 | Breast Cancer | 28.3 | 34.6 |
| (OD04451-02) | | | (OD04566) | | |
| Normal Prostate | 0.7 | 0.5 | Breast Cancer | 87.7 | 80.7 |
| 6546-1 | | | (OD04590-01) | | |
| Prostate Cancer | 1.1 | 0.9 | Breast Cancer | 100.0 | 100.0 |
| (OD04410) | | | Mets | | |
| | | | (OD04590-03) | | |
| Prostate Margin | 2.5 | 2.2 | Breast Cancer | 76.8 | 81.2 |
| (OD04410) | | | Metastasis | | |
| | | | (OD04655-05) | | |
| Prostate Cancer | 1.7 | 1.4 | Breast Cancer | 10.2 | 11.1 |
| (OD04720-01) | | | 064006 | | |
| Prostate Margin | 2.1 | 3.6 | Breast Cancer | 19.9 | 20.0 |
| (OD04720-02) | | | 1024 | | |
| Normal Lung | 7.0 | 11.1 | Breast Cancer | 26.2 | 31.6 |
| 061010 | | | 9100266 | | |
| Lung Met to | 0.4 | 0.9 | Breast Margin | 10.1 | 13.2 |
| Muscle | | | 9100265 | | |
| (ODO4286) | | | | | |
| Muscle Margin | 2.9 | 6.9 | Breast Cancer | 13.6 | 21.6 |
| (ODO4286) | | | A209073 | | |
| Lung Malignant | 2.0 | 3.2 | Breast Margin | 10.4 | 12.3 |
| Cancer | | | A209073 | | |
| (OD03126) | | | | | |
| Lung Margin | 3.6 | 6.5 | Normal Liver | 0.5 | 0.4 |
| (OD03126) | | | | | |
| Lung Cancer | 4.1 | 7.8 | Liver Cancer | 0.5 | 0.3 |
| (OD04404) | | | 064003 | | |
| Lung Margin | 19.3 | 20.0 | Liver Cancer | 0.2 | 0.3 |
| (OD04404) | | | 1025 | | |
| Lung Cancer | 0.4 | 0.7 | Liver Cancer | 1.9 | 2.7 |
| (OD04565) | | | 1026 | | |
| Lung Margin | 3.9 | 3.3 | Liver Cancer | 0.2 | 0.5 |
| (OD04565) | | | 6004-T | | |
| Lung Cancer | 3.0 | 4.8 | Liver Tissue | 0.2 | 0.3 |
| (OD04237-01) | | | 6004-N | | |
| Lung Margin | 8.0 | 8.1 | Liver Cancer | 2.0 | 2.2 |
| (OD04237-02) | | | 6005-T | | |
| Ocular Mel Met | 0.0 | 0.2 | Liver Tissue | 1.2 | 1.3 |
| to Liver | | | 6005-N | | |
| (ODO4310) | | | | | |
| Liver Margin | 0.6 | 1.0 | Normal | 3.1 | 3.3 |
| (ODO4310) | | | Bladder | | |
| Melanoma Mets | 1.5 | 2.6 | Bladder Cancer | 1.7 | 1.5 |
| to Lung | | | 1023 | | |
| (OD04321) | | | | | |
| Lung Margin | 6.4 | 9.8 | Bladder Cancer | 0.6 | 0.8 |
| (OD04321) | | | A302173 | | |
| Normal Kidney | 27.0 | 54.0 | Bladder Cancer | 0.8 | 0.9 |
| | | | (OD04718-01) | | |
| Kidney Ca, | 2.3 | 3.1 | Bladder | 42.6 | 53.6 |
| Nuclear grade 2 | | | Normal | | |
| (OD04338) | | | Adjacent | | |
| | | | (OD04718-03) | | |
| Kidney Margin | 11.7 | 16.0 | Normal Ovary | 3.3 | 4.0 |
| (OD04338) | | | | | |
| Kidney Ca | 7.5 | 7.1 | Ovarian | 15.0 | 18.9 |
| Nuclear grade 1/2 | | | Cancer 064008 | | |
| (OD04339) | | | | | |
| Kidney Margin | 14.9 | 16.8 | Ovarian | 1.3 | 1.7 |
| (OD04339) | | | Cancer | | |
| | | | (OD04768-07) | | |
| Kidney Ca, Clear | 1.5 | 2.3 | Ovary Margin | 9.8 | 9.7 |
| cell type | | | (OD04768-08) | | |
| (OD04340) | | | | | |
| Kidney Margin | 19.8 | 18.3 | Normal | 14.8 | 15.1 |
| (OD04340) | | | Stomach | | |
| Kidney Ca, | 0.8 | 1.2 | Gastric Cancer | 3.0 | 3.1 |
| Nuclear grade 3 | | | 9060358 | | |
| (OD04348) | | | | | |
| Kidney Margin | 13.5 | 19.3 | Stomach | 8.0 | 7.7 |
| (OD04348) | | | Margin | | |
| | | | 9060359 | | |
| Kidney Cancer | 1.5 | 1.9 | Gastric Cancer | 2.8 | 3.1 |
| (OD04622-01) | | | 9060395 | | |
| Kidney Margin | 4.7 | 5.8 | Stomach | 8.1 | 8.7 |
| (OD04622-03) | | | Margin | | |
| | | | 9060394 | | |
| Kidney Cancer | 0.1 | 0.2 | Gastric Cancer | 0.5 | 0.9 |
| (OD04450-01) | | | 9060397 | | |
| Kidney Margin | 11.1 | 17.7 | Stomach | 9.8 | 9.7 |
| (OD04450-03) | | | Margin | | |
| | | | 9060396 | | |
| Kidney Cancer | 2.1 | 3.1 | Gastric Cancer | 1.7 | 2.6 |
| 8120607 | | | 064005 | | |

TABLE EK
Panel 3D

| | Rel. Exp.(%) Ag554, | | Rel. Exp.(%) Ag554, |
|---|---|---|---|
| Tissue Name | Run 164886640 | Tissue Name | Run 164886640 |
| Daoy-Medulloblastoma | 18.3 | Ca Ski-Cervical epidermoid | 6.6 |
| | | carcinoma (metastasis) | |
| TE671-Medulloblastoma | 1.0 | ES-2-Ovarian clear cell | 0.9 |
| | | carcinoma | |
| D283 Med- | 1.8 | Ramos-Stimulated with | 0.0 |
| Medulloblastoma | | PMA/ionomycin 6h | |
| PFSK-1-Primitive | 0.0 | Ramos-Stimulated with | 0.0 |
| Neuroectodermal | | PMA/ionomycin 14h | |
| XF-498-CNS | 46.7 | MEG-01-Chronic | 0.0 |
| | | myelogenous leukemia | |
| | | (megokaryoblast) | |
| SNB-78-Glioma | 1.2 | Raji-Burkitt's lymphoma | 0.0 |
| SF-268-Glioblastoma | 3.5 | Daudi-Burkitt's lymphoma | 0.0 |
| T98G-Glioblastoma | 3.5 | U266-B-cell plasmacytoma | 0.0 |
| SK-N-SH-Neuroblastoma | 2.1 | CA46-Burkitt's lymphoma | 0.0 |
| (metastasis) | | | |
| SF-295-Glioblastoma | 2.9 | RL-non-Hodgkin's B-cell | 0.0 |
| | | lymphoma | |
| Cerebellum | 0.0 | JM1-pre-B-cell lymphoma | 0.0 |
| Cerebellum | 0.1 | Jurkat-T cell leukemia | 0.0 |
| NCI-H292- | 13.8 | TF-1-Erythroleukemia | 0.0 |
| Mucoepidermoid lung | | | |
| carcinoma | | | |
| DMS-114-Small cell lung | 1.1 | HUT 78-T-cell lymphoma | 0.0 |
| cancer | | | |
| DMS-79-Small cell lung | 77.9 | U937-Histiocytic lymphoma | 0.0 |
| cancer | | | |
| NCI-H146-Small cell | 0.0 | KU-812-Myelogenous | 0.0 |
| lung cancer | | leukemia | |
| NCI-H526-Small cell | 0.6 | 769-P-Clear cell renal | 2.8 |
| lung cancer | | carcinoma | |
| NCI-N417-Small cell | 0.0 | Caki-2-Clear cell renal | 2.4 |
| lung cancer | | carcinoma | |
| NCI-H82-Small cell lung | 3.1 | SW 839-Clear cell renal | 4.8 |
| cancer | | carcinoma | |
| NCI-H157-Squamous | 18.9 | G401-Wilms' tumor | 0.0 |
| cell lung cancer | | | |
| (metastasis) | | | |
| NCI-H1155-Large cell | 3.5 | Hs766T-Pancreatic carcinoma | 13.5 |
| lung cancer | | (LN metastasis) | |
| NCI-H1299-Large cell | 0.0 | CAPAN-1-Pancreatic | 2.2 |
| lung cancer | | adenocarcinoma (liver | |
| | | metastasis) | |
| NCI-H727-Lung | 27.9 | SU86.86-Pancreatic | 8.7 |
| carcinoid | | carcinoma (liver metastasis) | |
| NCI-UMC-11-Lung | 0.2 | BxPC-3-Pancreatic | 5.3 |
| carcinoid | | adenocarcinoma | |
| LX-1-Small cell lung | 2.9 | HPAC-Pancreatic | 13.9 |
| cancer | | adenocarcinoma | |
| Colo-205-Colon cancer | 2.0 | MIA PaCa-2-Pancreatic | 13.6 |
| | | carcinoma | |
| KM12-Colon cancer | 7.0 | CFPAC-1-Pancreatic ductal | 23.0 |
| | | adenocarcinoma | |
| KM20L2-Colon cancer | 0.0 | PANC-1-Pancreatic | 100.0 |
| | | epithelioid ductal carcinoma | |
| NCI-H716-Colon cancer | 0.3 | T24-Bladder carcinoma | 7.5 |
| | | (transitional cell) | |
| SW-48-Colon | 0.0 | 5637-Bladder carcinoma | 2.3 |
| adenocarcinoma | | | |
| SW1116-Colon | 0.7 | HT-1197-Bladder carcinoma | 0.3 |
| adenocarcinoma | | | |
| LS 174T-Colon | 1.4 | UM-UC-3-Bladder carcinma | 0.0 |
| adenocarcinoma | | (transitional cell) | |
| SW-948-Colon | 2.7 | A204-Rhabdomyosarcoma | 0.0 |
| adenocarcinoma | | | |
| SW-480-Colon | 0.0 | HT-1080-Fibrosarcoma | 5.7 |
| adenocarcinoma | | | |
| NCI-SNU-5-Gastric | 17.4 | MG-63-Osteosarcoma | 7.4 |
| carcinoma | | | |
| KATO III-Gastric | 0.2 | SK-LMS-1-Leiomyosarcoma | 10.8 |
| carcinoma | | (vulva) | |
| NCI-SNU-16-Gastric | 6.9 | SJRH30-Rhabdomyosarcoma | 8.2 |
| carcinoma | | (met to bone marrow) | |
| NCI-SNU-1-Gastric | 0.0 | A431-Epidermoid carcinoma | 6.3 |
| carcinoma | | | |
| RF-1-Gastric | 0.0 | WM266-4-Melanoma | 0.0 |
| adenocarcinoma | | | |
| RF-48-Gastric | 0.0 | DU 145-Prostate carcinoma | 0.0 |
| adenocarcinoma | | (brain metastasis) | |
| MKN-45-Gastric | 9.3 | MDA-MB-468-Breast | 0.0 |
| carcinoma | | adenocarcinoma | |
| NCI-N87-Gastric | 2.2 | SCC-4-Squamous cell | 0.0 |
| carcinoma | | carcinoma of tongue | |
| OVCAR-5-Ovarian | 6.5 | SCC-9-Squamous cell | 0.0 |
| carcinoma | | carcinoma of tongue | |
| RL95-2-Uterine | 0.2 | SCC-15-Squamous cell | 0.7 |
| carcinoma | | carcinoma of tongue | |
| HelaS3-Cervical | 0.9 | CAL 27-Squamous cell | 0.6 |
| adenocarcinoma | | carcinoma of tongue | |

TABLE EL
Panel 4.1D

| | Rel. | Rel. | | Rel. | Rel. |
|---|---|---|---|---|---|
| | Exp.(%) | Exp.(%) | | Exp.(%) | Exp.(%) |
| | Ag3340, | Ag3340, | | Ag3340, | Ag3340, |
| | Run | Run | | Run | Run |
| Tissue Name | 170737060 | 170766524 | Tissue Name | 170737060 | 170766524 |
| Secondary Th1 act | 0.2 | 0.0 | HUVEC IL-1 beta | 0.0 | 0.0 |
| Secondary Th2 act | 0.0 | 0.0 | HUVEC IFN | 0.0 | 0.0 |
| | | | gamma | | |
| Secondary Tr1 act | 0.0 | 0.0 | HUVEC TNF | 0.0 | 0.0 |
| | | | alpha + IFN | | |
| | | | gamma | | |
| SecondaryTh1 rest | 0.0 | 0.0 | HUVEC TNF | 1.2 | 1.8 |
| | | | alpha + IL4 | | |
| Secondary Th2 rest | 0.0 | 0.0 | HUVEC IL-11 | 0.2 | 0.0 |
| Secondary Tr1 rest | 0.0 | 0.0 | Lung | 1.7 | 2.0 |
| | | | Microvascular EC | | |
| | | | none | | |
| Primary Th1 act | 0.0 | 0.0 | Lung | 0.0 | 0.0 |
| | | | Microvascular EC | | |
| | | | TNF alpha + IL- | | |
| | | | 1 beta | | |
| Primary Th2 act | 0.0 | 0.0 | Microvascular | 0.5 | 1.2 |
| | | | Dermal EC none | | |
| Primary Tr1 act | 0.0 | 1.5 | Microsvasular | 0.0 | 0.0 |
| | | | Dermal EC | | |
| | | | TNF alpha + IL- | | |
| | | | 1 beta | | |
| Primary Th1 rest | 0.0 | 0.0 | Bronchial | 0.6 | 0.4 |
| | | | epithelium | | |
| | | | TNF alpha + | | |
| | | | IL1 beta | | |
| Primary Th2 rest | 0.0 | 0.0 | Small airway | 0.0 | 1.9 |
| | | | epithelium none | | |
| Primary Tr1 rest | 0.2 | 0.0 | Small airway | 0.4 | 0.3 |
| | | | epithelium | | |
| | | | TNF alpha + IL- | | |
| | | | 1 beta | | |
| CD45RA CD4 | 1.3 | 0.4 | Coronery artery | 2.6 | 7.6 |
| lymphocyte act | | | SMC rest | | |
| CD45RO CD4 | 0.0 | 0.0 | Coronery artery | 3.1 | 2.9 |
| lymphocyte act | | | SMC TNF alpha + | | |
| | | | IL-1 beta | | |
| CD8 lymphocyte | 0.0 | 0.0 | Astrocytes rest | 18.4 | 35.4 |
| act | | | | | |
| Secondary CD8 | 0.0 | 0.0 | Astrocytes | 7.0 | 11.7 |
| lymphocyte rest | | | TNF alpha + IL- | | |
| | | | 1 beta | | |
| Secondary CD8 | 0.0 | 0.0 | KU-812 (Basophil) | 0.0 | 0.0 |
| lymphocyte act | | | rest | | |
| CD4 lymphocyte | 0.0 | 0.0 | KU-812 (Basophil) | 0.4 | 0.0 |
| none | | | PMA/ionomycin | | |
| 2ry | 0.0 | 0.0 | CCD1106 | 10.3 | 8.5 |
| Th1/Th2/Tr1_anti- | | | (Keratinocytes) | | |
| CD95 CH11 | | | none | | |
| LAK cells rest | 0.0 | 0.0 | CCD1106 | 1.2 | 3.3 |
| | | | (Keratinocytes) | | |
| | | | TNF alpha + IL- | | |
| | | | 1 beta | | |
| LAK cells IL-2 | 0.4 | 0.0 | Liver cirrhosis | 0.3 | 0.9 |
| LAK cells IL-2 + | 0.0 | 0.0 | NCI-H292 none | 1.1 | 5.6 |
| IL-12 | | | | | |
| LAK cells IL- | 0.0 | 0.0 | NCI-H292 IL-4 | 28.1 | 42.3 |
| 2 + IFN gamma | | | | | |
| LAK cells IL-2 + | 0.0 | 0.0 | NCI-H292 IL-9 | 4.3 | 10.1 |
| IL-18 | | | | | |
| LAK cells | 0.0 | 0.4 | NCI-H292 IL-13 | 46.7 | 52.1 |
| PMA/ionomycin | | | | | |
| NK Cells IL-2 rest | 0.2 | 0.8 | NCI-H292 IFN | 0.8 | 8.1 |
| | | | gamma | | |
| Two Way MLR 3 | 0.0 | 0.0 | HPAEC none | 0.0 | 1.5 |
| day | | | | | |
| Two Way MLR 5 | 0.0 | 0.0 | HPAEC TNF | 0.0 | 3.9 |
| day | | | alpha + IL-1 beta | | |
| Two Way MLR 7 | 0.0 | 0.0 | Lung fibroblast | 76.3 | 74.7 |
| day | | | none | | |
| PBMC rest | 0.0 | 0.0 | Lung fibroblast | 0.8 | 2.2 |
| | | | TNF alpha + IL-1 | | |
| | | | beta | | |
| PBMC PWM | 0.0 | 0.0 | Lung fibroblast IL-4 | 100.0 | 92.7 |
| PBMC PHA-L | 0.0 | 0.3 | Lung fibroblast IL-9 | 48.3 | 48.0 |
| Ramos (B cell) | 0.0 | 0.0 | Lung fibroblast IL- | 100.0 | 100.0 |
| none | | | 13 | | |
| Ramos (B cell) | 0.0 | 0.0 | Lung fibroblast | 32.1 | 51.4 |
| ionomycin | | | IFN gamma | | |
| B lymphocytes | 0.0 | 0.0 | Dermal fibroblast | 4.5 | 7.4 |
| PWM | | | CCD1070 rest | | |
| B lymphocytes | 0.0 | 0.0 | Dermal fibroblast | 3.0 | 3.1 |
| CD40L and IL-4 | | | CCD1070 TNF | | |
| | | | alpha | | |
| EOL-1 dbcAMP | 0.0 | 0.0 | Dermal fibroblast | 1.8 | 2.2 |
| | | | CCD1070 IL-1 | | |
| | | | beta | | |
| EOL-1 dbcAMP | 0.0 | 0.0 | Dermal fibroblast | 2.7 | 6.5 |
| PMA/ionomycin | | | IFN gamma | | |
| Dendritic cells none | 0.6 | 0.0 | Dermal fibroblast | 40.9 | 43.2 |
| | | | IL-4 | | |
| Dendritic cells LPS | 0.2 | 0.5 | Dermal Fibroblasts | 8.0 | 9.2 |
| | | | rest | | |
| Dendritic cells anti- | 4.5 | 3.4 | Neutrophils | 1.1 | 0.9 |
| CD40 | | | TNFa + LPS | | |
| Monocytes rest | 0.0 | 0.0 | Neutrophils rest | 2.2 | 0.0 |
| Monocytes LPS | 0.0 | 0.0 | Colon | 3.5 | 2.3 |
| Macrophages rest | 0.9 | 1.1 | Lung | 7.0 | 8.5 |
| Macrophages LPS | 0.0 | 0.0 | Thymus | 5.0 | 12.8 |
| HUVEC none | 0.1 | 0.0 | Kidney | 21.2 | 36.1 |
| HUVEC starved | 0.3 | 0.0 | | | |

TABLE EM
Panel 4D

| | Rel. | Rel. | | Rel. | Rel. |
|---|---|---|---|---|---|
| | Exp. (%) | Exp. (%) | | Exp. (%) | Exp. (%) |
| | Ag3340, | Ag554, | | Ag3340, | Ag554, |
| | Run | Run | | Run | Run |
| Tissue Name | 165222713 | 164886924 | Tissue Name | 165222713 | 164886924 |
| Secondary Th1 act | 0.0 | 0.1 | HUVEC IL-1 beta | 0.0 | 0.0 |
| Secondary Th2 act | 0.0 | 0.1 | HUVEC IFN | 0.0 | 0.0 |
| | | | gamma | | |
| Secondary Tr1 act | 0.0 | 0.1 | HUVEC TNF | 0.0 | 0.0 |
| | | | alpha + IFN | | |
| | | | gamma | | |
| Secondary Th1 rest | 0.0 | 0.0 | HUVEC TNF | 0.6 | 0.5 |
| | | | alpha + IL4 | | |
| Secondary Th2 rest | 0.0 | 0.0 | HUVEC IL-11 | 0.1 | 0.0 |
| Secondary Tr1 rest | 0.1 | 0.0 | Lung | 0.4 | 0.9 |
| | | | Microvascular EC | | |
| | | | none | | |
| Primary Th1 act | 0.0 | 0.0 | Lung | 0.1 | 0.2 |
| | | | Microvascular EC | | |
| | | | TNF alpha + IL- | | |
| | | | 1 beta | | |
| Primary Th2 act | 0.0 | 0.0 | Microvascular | 0.3 | 0.6 |
| | | | Dermal EC none | | |
| Primary Tr1 act | 0.0 | 0.0 | Microsvasular | 0.0 | 0.1 |
| | | | Dermal EC | | |
| | | | TNF alpha + IL- | | |
| | | | 1 beta | | |
| Primary Th1 rest | 0.0 | 0.0 | Bronchial | 0.0 | 0.4 |
| | | | epithelium | | |
| | | | TNF alpha + | | |
| | | | IL1 beta | | |
| Primary Th2 rest | 0.0 | 0.0 | Small airway | 0.5 | 0.2 |
| | | | epithelium none | | |
| Primary Tr1 rest | 0.3 | 0.0 | Small airway | 0.4 | 1.0 |
| | | | epithelium | | |
| | | | TNF alpha + IL- | | |
| | | | 1 beta | | |
| CD45RA CD4 | 0.1 | 0.4 | Coronery artery | 2.7 | 3.5 |
| lymphocyte act | | | SMC rest | | |
| CD45RO CD4 | 0.0 | 0.0 | Coronery artery | 1.2 | 0.8 |
| lymphocyte act | | | SMC TNF alpha + | | |
| | | | IL-1 beta | | |
| CD8 lymphocyte | 0.0 | 0.0 | Astrocytes rest | 19.5 | 21.8 |
| act | | | | | |
| Secondary CD8 | 0.0 | 0.0 | Astrocytes | 7.0 | 6.9 |
| lymphocyte rest | | | TNF alpha + IL- | | |
| | | | 1 beta | | |
| Secondary CD8 | 0.0 | 0.0 | KU-812 (Basophil) | 0.0 | 0.0 |
| lymphocyte act | | | rest | | |
| CD4 lymphocyte | 0.0 | 0.0 | KU-812 (Basophil) | 0.0 | 0.0 |
| none | | | PMA/ionomycin | | |
| 2ry | 0.1 | 0.1 | CCD1106 | 2.8 | 3.4 |
| Th1/Th2/Tr1_anti- | | | (Keratinocytes) | | |
| CD95 CH11 | | | none | | |
| LAK cells rest | 0.0 | 0.0 | CCD1106 | 2.3 | 1.3 |
| | | | (Keratinocytes) | | |
| | | | TNF alpha + IL- | | |
| | | | 1 beta | | |
| LAK cells IL-2 | 0.0 | 0.0 | Liver cirrhosis | 0.6 | 1.6 |
| LAK cells IL-2 + | 0.1 | 0.0 | Lupus kidney | 0.5 | 0.9 |
| IL-12 | | | | | |
| LAK cells IL- | 0.0 | 0.0 | NCI-H292 none | 2.7 | 2.7 |
| 2 + IFN gamma | | | | | |
| LAK cells IL-2 + | 0.1 | 0.1 | NCI-H292 IL-4 | 33.4 | 39.5 |
| IL-18 | | | | | |
| LAK-cells | 0.1 | 0.0 | NCI-H292 IL-9 | 4.6 | 3.5 |
| PMA/ionomycin | | | | | |
| NK Cells IL-2 rest | 0.1 | 0.2 | NCI-H292 IL-13 | 31.0 | 26.1 |
| Two Way MLR 3 | 0.0 | 0.0 | NCI-H292 IFN | 3.4 | 3.6 |
| day | | | gamma | | |
| Two Way MLR 5 | 0.0 | 0.2 | HPAEC none | 0.0 | 0.0 |
| day | | | | | |
| Two Way MLR 7 | 0.0 | 0.0 | HPAEC TNF | 0.0 | 0.0 |
| day | | | alpha + IL-1 beta | | |
| PBMC rest | 0.0 | 0.0 | Lung fibroblast | 36.9 | 53.6 |
| | | | none | | |
| PBMC PWM | 0.0 | 0.0 | Lung fibroblast | 0.6 | 0.8 |
| | | | TNF alpha + IL-1 | | |
| | | | beta | | |
| PBMC PHA-L | 0.0 | 0.0 | Lung fibroblast IL-4 | 100.0 | 100.0 |
| Ramos (B cell) | 0.0 | 0.0 | Lung fibroblast IL-9 | 26.8 | 29.7 |
| none | | | | | |
| Ramos (B cell) | 0.0 | 0.0 | Lung fibroblast IL- | 75.8 | 74.2 |
| ionomycin | | | 13 | | |
| B lymphocytes | 0.0 | 0.0 | Lung fibroblast | 32.8 | 36.6 |
| PWM | | | IFN gamma | | |
| B lymphocytes | 0.0 | 0.0 | Dermal fibroblast | 2.5 | 5.6 |
| CD40L and IL-4 | | | CCD1070 rest | | |
| EOL-1 dbcAMP | 0.0 | 0.0 | Dermal fibroblast | 0.9 | 2.8 |
| | | | CCD1070 TNF | | |
| | | | alpha | | |
| EOL-1 dbcAMP | 0.1 | 0.0 | Dermal fibroblast | 0.4 | 0.4 |
| PMA/ionomycin | | | CCD1070 IL-1 | | |
| | | | beta | | |
| Dendritic cells none | 0.2 | 0.6 | Dermal fibroblast | 0.6 | 2.8 |
| | | | IFN gamma | | |
| Dendritic cells LPS | 0.0 | 0.1 | Dermal fibroblast | 26.6 | 29.9 |
| | | | IL-4 | | |
| Dendritic cells anti- | 1.2 | 2.0 | IBD Colitis 2 | 0.4 | 0.7 |
| CD40 | | | | | |
| Monocytes rest | 0.0 | 0.0 | IBD Crohn's | 1.6 | 1.5 |
| Monocytes LPS | 0.0 | 0.0 | Colon | 1.8 | 2.5 |
| Macrophages rest | 0.3 | 0.9 | Lung | 4.8 | 8.5 |
| Macrophages LPS | 0.0 | 0.0 | Thymus | 16.3 | 24.7 |
| HUVEC none | 0.0 | 0.0 | Kidney | 3.4 | 3.8 |
| HUVEC starved | 0.0 | 0.4 | | | |

AI_comprehensive panel_v1.0 Summary: Ag3340 The transcript is highly expressed in 3 out of 6 ulcerative colitis samples, but not in the matched control samples. Similarly, it is expressed in OA tissue and not in normal control samples. Asthma and emphysema lung samples express the transcript at higher levels than in normal lung.

The protein encoded for by CG57829-01 has homology to ADAMTS family of molecules suggesting that it may function as an enzyme. Based on its homology, it may contribute to the tissue destruction and remodeling processes associated with asthma, ulcerative colitis, emphysema and osteoarthritis. Therefore, blocking the function of the protein encoded for by CG57829-01 with antagonistic antibody therapeutics or small molecule therapeutics could reduce or inhibit tissue destruction in the lungs, intestine, or joints due to emphysema, allergy, asthma, colitis, or osteoarthritis.

CNS_neurodegeneration_v1.0 Summary: Ag3340 This panel does not show differential expression of the CG57829-01 gene in Alzheimer's disease. However, this expression profile confirms the presence of this gene in the brain. Please see Panel 1.4 for discussion of utility of this gene in the central nervous system.

General_screening_panel_v1.4 Summary: Ag3340 Two experiments with the same probe and primer set are in excellent agreement. Highest expression of the CG57829-01 gene is seen in breast cancer MDA-MB-231 cell line (CTs=25). As seen in Panel 1.1, 1.2, and 1.3, this gene has high expression in ovarian and breast cancer cell lines which is also confirmed in Panels 2.1 and 2D. Therefore, expression of this gene could be used as a diagnostic marker for the presence of these cancers. Furhtermore, therapeutic modulation of the activity of this gene or its protein product, through the use of small molecule drugs, protein therapeutics or antibodies, might be beneficial in the treatment of breast or ovarian cancer.

Among tissues with metabolic or endocrine function, this gene is expressed at low to moderate levels in pancreas, adipose, adrenal gland, thyroid, pituitary gland, skeletal muscle, heart, fetal liver and the gastrointestinal tract. Therefore, therapeutic modulation of the activity of this gene may prove useful in the treatment of endocrine/metabolically related diseases, such as obesity and diabetes.

Interestingly, this gene is expressed at much higher levels in fetal (CT=32) when compared to adult liver (CT=36). This observation suggests that expression of this gene can be used to distinguish fetal from adult liver.

In addition, this gene is expressed at low to moderate levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

This gene encodes a homologue of rat ADAMTS-1. Members belonging to ADAM and ADAMTS family has been found to play a role in various inflammatory processes such as arthritic diseases, as well as, development of cancer cachexia and thrombotic thrombocytopenic purpura (Ref. 1, 2, 3). Therefore, therapeutic modulation of the activity of this gene or its protein product, through the use of small molecule drugs, protein therapeutics or antibodies, might be beneficial in the treatment of these diseases.

References.

1. Martel-Pelletier J, Welsch D J, Pelletier J P. (2001) Metalloproteases and inhibitors in arthritic diseases. Best Pract Res Clin Rheumatol 15(5):805-29

2. Kuno K, Kanada N, Nakashima E, Fujiki F, Ichimura F, Matsushima K. (1 997) Molecular cloning of a gene encoding a new type of metalloproteinase-disintegrin family protein with thrombosponidin motifs as an inflammation associated gene. J Biol Chem 272(1):556-62.

3. Levy G G, Nichols W C, Lian E C, Foroud T, McClintick J N, McGee B M, Yang A Y, Siemieniak D R, Stark K R, Gnippo R, Sarode R, Shurin S B, Chandrasekaran V, Stabler S P, Sabio H, Bouhassira E E, Upshaw J D Jr, Ginsburg D, Tsai H M. (2001) Mutations in a member of the ADAMTS gene family cause thrombotic thrombocytopenic purpura. Nature 413(6855):488-94

Panel 1.1 Summary: Ag554 Highest expression of the CG57829-01 gene is seen in ovarian cancer OVCAR-3 cell line (CT=22). Expression pattern of this gene in this panel is similar to that of panel 1.4. Please see panel 1.4 for discussion and potential utility of this gene.

Panel 1.2 Summary: Ag554 Highest expression of the CG57829-01 gene is seen in fetal kidney (CT=23.8). Interestingly, this gene is expressed at much higher levels in fetal (CT=23.8) when compared to adult kidney (CT=27-31). This observation suggests that expression of this gene can be used to distinguish fetal from adult kidney.

High expression of this gene is also seen in cluster of ovarian cancer, breat cancer, renal cancer, prostate cancer, CNS cancer cell lines, as well as in placenta, mammary glands, liver, trachea, stomach, colorectal, and heart. Please see panel 1.4 for discussion and potential utility of this gene.

Panel 1.3D Summary: Ag554 Highest expression of the CG57829-01 gene is seen in breast cancer MDA-MB-231 cell line (CT=27.8). Expression pattern of this gene in this panel is similar to that of panel 1.4. Please see panel 1.4 for discussion and potential utility of this gene.

Panel 2.1 Summary: Ag3340 Highest expression of the CG57829-01 gene is seen in breast cancer metastasis sample (CT=28). As seen in panel 1.4, expression of this gene is associated with most cancers. Interestingly, expression of this gene seems to be down-regulated in kidney cancers (OD04340, OD04348, OD04450-01, 8120614) and colon metastasis to lung (OD04451-01) (CTs=35-40) compared to the marginal control samples (CTs=31-33). Therefore, expression of this gene can be used as potential marker to distinguishing between these cancer and normal tissues.

Please see panel 1.4 for discussion and potential utility of this gene.

Panel 2D Summary: Ag554 Highest expression of the CG57829-01 gene is seen in breast cancer metastasis (OD04590-03) sample (CT=27). As seen in panel 1.4, expression of this gene is associated with most cancers, with high expression in breast cancer samples. Please see panel 1.4 for discussion and potential utility of this gene.

Panel 3D Summary: Ag554 Highest expression of the CG57829-01 gene is seen in PANC-1-Pancreatic epithelioid ductal carcinoma sample (CT=28). As seen in panel 1.4, expression of this gene is associated with most cancers, with high expression in XF-CNS, small cell lung and pancreatic cancers. Please see panel 1.4 for discussion and potential utility of this gene.

Panel 4.1D Summary: Ag3340 CG57829-01 is highly induced in the mucoepidermoid cell line NCI-H292 by IL-4 and IL-13 (CTs=29-30). Expression is also high in lung fibroblasts treated with IL-4 or IL-13 and dermal fibroblasts treated with IL-4 (CTs=28-29). These findings are consistent in 3 separate runs with two sets of primers and probes.

Potential Role(s) of CG57829-01 in Inflammation: The protein encoded for by CG57892-01 has homology to ADAMTS family of molecules suggesting that it may function as an enzyme (see references in panel 1.4). The expression of this transcript by lung fibroblasts, the goblet cell like cell line NCI-H292 and dermal fibroblasts, particularly after exposure to IL-4 and IL-13 is consistent with it participating in diseases such as asthma, psoriasis, emphysema and arthritis (Ref. 1, 2). Based on its homology, it may contribute to the tissue destruction and remodeling processes associated with asthma, psoriasis, emphysema and arthritis.

Blocking the function of the protein encoded for by CG57892-01 with antagonistic antibody therapeutics could reduce or inhibit tissue destruction in the lungs, skin, or joints due to emphysema, allergy, asthma, psoriasis, or arthritis.

References.

1. Laliberte R, Rouabhia M, Bosse M, Chakir J. Decreased capacity of asthmatic bronchial fibroblasts to degrade collagen. Matrix Biol January 2001;19(8):743-53

2. Vankemmelbeke M N, Holen I, Wilson A, Ilic M Z, Handley C J, Kelner G S, Clark M, Liu C, Maki R A, Burnett D, Buttle D J. Expression and activity of ADAMTS-5 in synovium. Eur J Biochem March 2001;268(5):1259-68

Panel 4D Summary: Ag554/Ag3340 Expression pattern for the CG57829-01 gene is same as seen in panel 4.1D with highest expression in IL-4 Stimulated lung fibroblast (CT=26.7). Please see the panel 4.1D for discussion and potential utility of this gene.

### F. NOV6a (CG59197-01: TULIP 2)

Expression of gene CG59197-01 was assessed using the primier-probe set Ag3391, described in Table FA. Results of the RTQ-PCR runs are shown in Tables FB, FC and FD.

TABLE FA
Probe Name Ag3391

| | | | | Start |
|---|---|---|---|---|
| Primers | Sequences | | Length | Position |
| Forward | 5′-cctgaactctttgagagtccaa-3′ | (SEQ ID NO:230) | 22 | 1146 |
| Probe | TET-5′-caaccttagtgtcctgtatccagatca-3′-TAMRA | (SEQ ID NO:231) | 27 | 1195 |
| Reverse | 5′-ctccaggcatattctcttctga-3′ | (SEQ ID NO:232) | 22 | 1224 |

TABLE FB
CNS_neurodegeneration_v1.0

| | Rel. Exp.(%) Ag3391, | | Rel. Exp.(%) Ag3391, |
|---|---|---|---|
| Tissue Name | Run 210349173 | Tissue Name | Run 210349173 |
| AD 1 Hippo | 9.5 | Control (Path) 3 | 5.7 |
| | | Temporal Ctx | |
| AD 2 Hippo | 24.5 | Control (Path) 4 | 33.0 |
| | | Temporal Ctx | |
| AD 3 Hippo | 5.4 | AD 1 Occipital Ctx | 15.0 |
| AD 4 Hippo | 4.3 | AD 2 Occipital Ctx | 0.0 |
| | | (Missing) | |
| AD 5 Hippo | 85.9 | AD 3 Occipital Ctx | 5.4 |
| AD 6 Hippo | 46.7 | AD 4 Occipital Ctx | 18.8 |
| Control 2 Hippo | 22.5 | AD 5 Occipital Ctx | 38.2 |
| Control 4 Hippo | 8.6 | AD 6 Occipital Ctx | 34.4 |
| Control (Path) 3 | 9.4 | Control 1 Occipital | 6.8 |
| Hippo | | Ctx | |
| AD 1 Temporal Ctx | 19.3 | Control 2 Occipital | 60.7 |
| | | Ctx | |
| AD 2 Temporal Ctx | 32.3 | Control 3 Occipital | 17.9 |
| | | Ctx | |
| AD 3 Temporal Ctx | 5.7 | Control 4 Occipital | 7.3 |
| | | Ctx | |
| AD 4 Temporal Ctx | 19.9 | Control (Path) 1 | 97.3 |
| | | Occipital Ctx | |
| AD 5 Inf Temporal | 100.0 | Control (Path) 2 | 12.2 |
| Ctx | | Occipital Ctx | |
| AD 5 Sup Temporal | 43.5 | Control (Path) 3 | 5.1 |
| Ctx | | Occipital Ctx | |
| AD 6 Inf Temporal | 53.2 | Control (Path) 4 | 16.6 |
| Ctx | | Occipital Ctx | |
| AD 6 Sup Temporal | 59.9 | Control 1 Parietal | 8.9 |
| Ctx | | Ctx | |
| Control 1 Temporal | 7.0 | Control 2 Parietal | 41.8 |
| Ctx | | Ctx | |
| Control 2 Temporal | 35.6 | Control 3 Parietal | 17.6 |
| Ctx | | Ctx | |
| Control 3 Temporal | 14.6 | Control (Path) 1 | 73.7 |
| Ctx | | Parietal Ctx | |
| Control 3 Temporal | 8.7 | Control (Path) 2 | 26.2 |
| Ctx | | Parietal Ctx | |
| Control (Path) 1 | 57.0 | Control (Path) 3 | 6.7 |
| Temporal Ctx | | Parietal Ctx | |
| Control (Path) 2 | 36.9 | Control (Path) 4 | 44.4 |
| Temporal Ctx | | Parietal Ctx | |

TABLE FC
General_screening_panel v1.4

| | Rel. Exp.(%) Ag3391, | | Rel. Exp.(%) Ag3391, |
|---|---|---|---|
| Tissue Name | Run 216822195 | Tissue Name | Run 216822195 |
| Adipose | 12.9 | Renal ca. TK-10 | 25.9 |
| Melanoma* | 14.7 | Bladder | 30.6 |
| Hs688(A).T | | | |
| Melanoma* | 14.3 | Gastric ca. (liver met.) | 30.6 |
| Hs688(B).T | | NCI-N87 | |
| Melanoma* M14 | 9.0 | Gastric ca. KATO III | 24.1 |
| Melanoma* | 12.0 | Colon ca. SW-948 | 5.9 |
| LOXIMVI | | | |
| Melanoma* SK- | 30.1 | Colon ca. SW480 | 24.1 |
| MEL-5 | | | |
| Squamous cell | 6.9 | Colon ca.* (SW480 | 20.9 |
| carcinoma SCC-4 | | met) SW620 | |
| Testis Pool | 16.0 | Colon ca. HT29 | 7.2 |
| Prostate ca.* (bone | 35.6 | Colon ca. HCT-116 | 39.8 |
| met) PC-3 | | | |
| Prostate Pool | 16.5 | Colon ca. CaCo-2 | 24.1 |
| Placenta | 3.0 | Colon cancer tissue | 12.4 |
| Uterus Pool | 16.8 | Colon ca. SW1116 | 7.6 |
| Ovarian ca. OVCAR-3 | 24.5 | Colon ca. Colo-205 | 2.2 |
| Ovarian ca. SK-OV-3 | 54.3 | Colon ca. SW-48 | 2.1 |
| Ovarian ca. OVCAR-4 | 2.2 | Colon Pool | 36.9 |
| Ovarian ca. OVCAR-5 | 49.0 | Small Intestine Pool | 44.4 |
| Ovarian ca. IGROV-1 | 21.9 | Stomach Pool | 21.5 |
| Ovarian ca. OVCAR-8 | 10.4 | Bone Marrow Pool | 18.8 |
| Ovary | 13.9 | Fetal Heart | 13.7 |
| Breast ca. MCF-7 | 46.3 | Heart Pool | 18.0 |
| Breast ca. MDA- | 22.4 | Lymph Node Pool | 48.6 |
| MB-231 | | | |
| Breast ca. BT 549 | 44.8 | Fetal Skeletal Muscle | 8.7 |
| Breast ca. T47D | 100.0 | Skeletal Muscle Pool | 21.2 |
| Breast ca. MDA-N | 9.3 | Spleen Pool | 17.9 |
| Breast Pool | 40.9 | Thymus Pool | 23.7 |
| Trachea | 17.4 | CNS cancer (glio/astro) | 18.6 |
| | | U87-MG | |
| Lung | 11.3 | CNS cancer (glio/astro) | 40.6 |
| | | U-118-MG | |
| Fetal Lung | 57.4 | CNS cancer (neuro;met) | 38.2 |
| | | SK-N-AS | |
| Lung ca. NCI-N417 | 6.2 | CNS cancer (astro) SF- | 7.5 |
| | | 539 | |
| Lung ca. LX-1 | 27.9 | CNS cancer (astro) | 35.6 |
| | | SNB-75 | |
| Lung ca. NCI-H146 | 8.7 | CNS cancer (glio) SNB- | 21.3 |
| | | 19 | |
| Lung ca. SHP-77 | 46.7 | CNS cancer (glio) SF- | 35.6 |
| | | 295 | |
| Lung ca. A549 | 30.4 | Brain (Amygdala) Pool | 28.1 |
| Lung ca. NCI-H526 | 5.8 | Brain (cerebellum) | 33.7 |
| Lung ca. NCI-H23 | 50.0 | Brain (fetal) | 65.5 |
| Lung ca. NCI-H460 | 25.0 | Brain (Hippocampus) | 29.3 |
| | | Pool | |
| Lung ca. HOP-62 | 10.8 | Cerebral Cortex Pool | 37.1 |
| Lung ca. NCI-H522 | 26.2 | Brain (Substantia nigra) | 33.0 |
| | | Pool | |
| Liver | 0.6 | Brain (Thalamus) Pool | 49.3 |
| Fetal Liver | 15.5 | Brain (whole) | 21.2 |
| Liver ca. HepG2 | 45.1 | Spinal Cord Pool | 23.5 |
| Kidney Pool | 70.2 | Adrenal Gland | 16.3 |
| Fetal Kidney | 28.3 | Pituitary gland Pool | 10.7 |
| Renal ca. 786-0 | 12.2 | Salivary Gland | 3.4 |
| Renal ca. A498 | 3.6 | Thyroid (female) | 16.2 |
| Renal ca. ACHN | 8.4 | Pancreatic ca. CAPAN2 | 18.8 |
| Renal ca. UO-31 | 14.4 | Pancreas Pool | 47.6 |

TABLE FD
Panel 4D

| | Rel. Exp.(%) Ag3391, | | Rel. Exp.(%) Ag3391, |
|---|---|---|---|
| Tissue Name | Run 165296469 | Tissue Name | Run 165296469 |
| Secondary Th1 act | 16.2 | HUVEC IL-1 beta | 8.4 |
| Secondary Th2 act | 20.3 | HUVEC IFN gamma | 17.3 |
| Secondary Tr1 act | 17.2 | HUVEC TNF alpha + IFN | 9.0 |
| | | gamma | |
| Secondary Th1 rest | 8.8 | HUVEC TNF alpha + IL4 | 8.2 |
| Secondary Th2 rest | 7.3 | HUVEC IL-11 | 4.4 |
| Secondary Tr1 rest | 9.6 | Lung Microvascular EC | 14.5 |
| | | none | |
| Primary Th1 act | 25.7 | Lung Microvascular EC | 10.6 |
| | | TNF alpha + IL-1 beta | |
| Primary Th2 act | 24.8 | Microvascular Dermal EC | 14.9 |
| | | none | |
| Primary Tr1 act | 39.2 | Microsvasular Dermal EC | 10.3 |
| | | TNF alpha + IL-1 beta | |
| Primary Th1 rest | 54.3 | Bronchial epithelium | 17.9 |
| | | TNF alpha + IL1 beta | |
| Primary Th2 rest | 31.6 | Small airway epithelium | 5.7 |
| | | none | |
| Primary Tr1 rest | 29.9 | Small airway epithelium | 43.5 |
| | | TNF alpha + IL-1 beta | |
| CD45RA CD4 | 8.7 | Coronery artery SMC rest | 11.0 |
| lymphocyte act | | | |
| CD45RO CD4 | 16.6 | Coronery artery SMC | 6.4 |
| lymphocyte act | | TNF alpha + IL-1 beta | |
| CD8 lymphocyte act | 12.7 | Astrocytes rest | 8.8 |
| Secondary CD8 | 13.6 | AstrocytesTNF alpha + IL- | 7.3 |
| lymphocyte rest | | 1 beta | |
| Secondary CD8 | 9.5 | KU-812 (Basophil) rest | 4.9 |
| lymphocyte act | | | |
| CD4 lymphocyte none | 7.6 | KU-812 (Basophil) | 26.8 |
| | | PMA/ionomycin | |
| 2ry Th1/Th2/Tr1_anti- | 12.9 | CCD1106 (Keratinocytes) | 8.0 |
| CD95 CH11 | | none | |
| LAK cells rest | 14.5 | CCD1106 (Keratinocytes) | 3.9 |
| | | TNF alpha + IL-1 beta | |
| LAK cells IL-2 | 16.4 | Liver cirrhosis | 4.0 |
| LAK cells IL-2 + IL-12 | 18.7 | Lupus kidney | 3.6 |
| LAK cells IL-2 + IFN | 36.9 | NCI-H292 none | 29.9 |
| gamma | | | |
| LAK cells IL-2 + IL-18 | 33.4 | NCI-H292 1L-4 | 50.3 |
| LAK cells | 18.9 | NCI-H292 IL-9 | 34.2 |
| PMA/ionomycin | | | |
| NK Cells IL-2 rest | 18.4 | NCI-H292 IL-13 | 17.0 |
| Two Way MLR 3 day | 24.3 | NCI-H292 IFN gamma | 17.3 |
| Two Way MLR 5 day | 10.3 | HPAEC none | 8.0 |
| Two Way MLR 7 day | 7.5 | HPAEC TNF alpha + IL-1 | 13.2 |
| | | beta | |
| PBMC rest | 8.7 | Lung fibroblast none | 7.0 |
| PBMC PWM | 61.1 | Lung fibroblast TNF | 5.3 |
| | | alpha + IL-1 beta | |
| PBMC PHA-L | 14.9 | Lung fibroblast IL-4 | 14.6 |
| Ramos (B cell) none | 19.1 | Lung fibroblast IL-9 | 15.8 |
| Ramos (B cell) | 100.0 | Lung fibroblast IL-13 | 9.7 |
| ionomycin | | | |
| B lymphocytes PWM | 65.1 | Lung fibroblast IFN | 17.4 |
| | | gamma | |
| B lymphocytes CD40L | 24.7 | Dermal fibroblast | 21.5 |
| and IL-4 | | CCD1070 rest | |
| EOL-1 dbcAMP | 7.7 | Dermal fibroblast | 63.7 |
| | | CCD1070 TNF alpha | |
| EOL-1 dbcAMP | 12.4 | Dermal fibroblast | 8.5 |
| PMA/ionomycin | | CCD1070 IL-1 beta | |
| Dendritic cells none | 13.1 | Dermal fibroblast IFN | 6.1 |
| | | gamma | |
| Dendritic cells LPS | 19.2 | Dermal fibroblast IL-4 | 14.8 |
| Dendritic cells anti- | 12.3 | IBD Colitis 2 | 2.1 |
| CD40 | | | |
| Monocytes rest | 14.8 | IBD Crohn's | 3.0 |
| Monocytes LPS | 29.9 | Colon | 23.2 |
| Macrophages rest | 18.8 | Lung | 11.0 |
| Macrophages LPS | 18.8 | Thymus | 34.9 |
| HUVEC none | 12.5 | Kidney | 25.2 |
| HUVEC starved | 28.9 | | |

CNS_neurodegeneration_v1.0 Summary: Ag3391 This panel confirms the expression of the CG59197-01 gene at significant levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3391 The CG59197-01 gene codes for a homologue of rat tuberin like protein, Tulip 2. Expression of this gene is ubiquitous in this panel, with highest expression in a breast cancer T47D cell line (CT=27). Expression of this gene appears to be higher in samples derived from breast, ovarian, prostate and melanoma cancer cell lines than in normal tissues. The widespread expression suggests that this gene product is involved in cell growth and proliferation. Thus, therapeutic modulation of the expression or function of this gene may be useful in the treatment of cancer.

Among tissues with metabolic or endocrine function, this gene is expressed at low to moderate levels in pancreas, adipose, adrenal gland, thyroid, pituitary gland, skeletal muscle, heart, liver and the gastrointestinal tract (CTs=28-34). Therefore, therapeutic modulation of the activity of this gene may prove useful in the treatment of endocrine/metabolically related diseases, such as obesity, and Types 1 and 2 diabetes.

Interestingly, this gene is expressed at much higher levels in fetal (CT=29.7) when compared to adult liver (CT=34.5). This observation suggests that expression of this gene can be used to distinguish fetal from adult liver.

In addition, this gene is expressed at high levels in all regions of the central nervous system examined, including amygdala, hippocampus, substantia nigra, thalamus, cerebellum, cerebral cortex, and spinal cord (CTs=27-29). Therefore, this gene may play a role in central nervous system disorders such as Alzheimer's disease, Parkinson's disease, epilepsy, multiple sclerosis, schizophrenia and depression.

Panel 4D Summary: Ag3391 Highest expression of the CG59197-01 gene is seen in ionomycin treated Ramos B cells (CT=27.6). This gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

In addition, expression of this gene is up-regulated in TNFalpha+IL-1beta-treated small airway epithelium, and PWM-treated PBMC (CTs=28) as compared to the untreated counterparts (CTs=31). Thus, expression of this gene may be used to distinguish between cytokine-treated small airway epithelium and PWM-treated PBMC cells from their untreated counterparts.

### G. NOV9a (CC58180-01: PROHIBITION)

Expression of gene CG58180-01 was assessed using the primer-probe set Ag3515, described in Table GA. Results of the RTQ-PCR runs are shown in Tables GB, GC and GD.

TABLE GA
Probe Name Ag3515

| | | | | Start |
|---|---|---|---|---|
| Primers | Sequences | | Length | Position |
| Forward | 5′-gcctttctccaccacgtaa-3′ | (SEQ ID NO:233) | 19 | 223 |
| Probe | TET-5′-tgtaccaatcatcactggtagcaaaga-3′-TAMRA | (SEQ ID NO:234) | 27 | 242 |
| Reverse | 5′-caacaggctggaagatgatg-3′ | (SEQ ID NO:235) | 20 | 296 |

TABLE GB
CNS_neurodegeneration_v1.0

| | Rel. Exp.(%) Ag3515, | | Rel. Exp.(%) Ag3515, |
|---|---|---|---|
| Tissue Name | Run 210499747 | Tissue Name | Run 210499747 |
| AD 1 Hippo | 12.4 | Control (Path) 3 | 6.5 |
| | | Temporal Ctx | |
| AD 2 Hippo | 26.8 | Control (Path) 4 | 40.1 |
| | | Temporal Ctx | |
| AD 3 Hippo | 8.2 | AD 1 Occipital Ctx | 18.7 |
| AD 4 Hippo | 11.0 | AD 2 Occipital Ctx | 0.0 |
| | | (Missing) | |
| AD 5 hippo | 71.7 | AD 3 Occipital Ctx | 19.3 |
| AD 6 Hippo | 100.0 | AD 4 Occipital Ctx | 36.6 |
| Control 2 Hippo | 15.3 | AD 5 Occipital Ctx | 15.1 |
| Control 4 Hippo | 21.9 | AD 6 Occipital Ctx | 31.4 |
| Control (Path) 3 | 12.9 | Control 1 Occipital | 6.4 |
| Hippo | | Ctx | |
| AD 1 Temporal Ctx | 26.4 | Control 2 Occipital | 33.0 |
| | | Ctx | |
| AD 2 Temporal Ctx | 42.6 | Control 3 Occipital | 45.7 |
| | | Ctx | |
| AD 3 Temporal Ctx | 14.9 | Control 4 Occipital | 5.0 |
| | | Ctx | |
| AD 4 Temporal Ctx | 48.3 | Control (Path) 1 | 95.3 |
| | | Occipital Ctx | |
| AD 5 Inf Temporal Ctx | 73.7 | Control (Path) 2 | 27.7 |
| Ctx | | Occipital Ctx | |
| AD 5 Sup Temporal | 55.5 | Control (Path) 3 | 3.5 |
| Ctx | | Occipital Ctx | |
| AD 6 Inf Temporal | 47.3 | Control (Path) 4 | 36.1 |
| Ctx | | Occipital Ctx | |
| AD 6 Sup Temporal | 69.7 | Control 1 Parietal | 19.8 |
| Ctx | | Ctx | |
| Control 1 Temporal | 12.2 | Control 2 Parietal | 50.0 |
| Ctx | | Ctx | |
| Control 2 Temporal | 11.0 | Control 3 Parietal | 18.4 |
| Ctx | | Ctx | |
| Control 3 Temporal | 13.8 | Control (Path) 1 | 47.0 |
| Ctx | | Parietal Ctx | |
| Control 4 Temporal | 11.7 | Control (Path) 2 | 48.0 |
| Ctx | | Parietal Ctx | |
| Control (Path) 1 | 51.4 | Control (Path) 3 | 5.6 |
| Temporal Ctx | | Parietal Ctx | |
| Control (Path) 2 | 55.9 | Control (Path) 4 | 56.3 |
| Temporal Ctx | | Parietal Ctx | |

TABLE GC
General_screening_panel_v1.4

| | Rel. Exp.(%) Ag3515, | | Rel. Exp.(%) Ag3515, |
|---|---|---|---|
| Tissue Name | Run 216709850 | Tissue Name | Run 216709850 |
| Adipose | 3.6 | Renal ca. TK-10 | 21.6 |
| Melanoma* | 12.5 | Bladder | 46.3 |
| Hs688(A).T | | | |
| Melanoma* | 12.0 | Gastric ca. (liver met.) | 57.4 |
| Hs688(B).T | | NCI-N87 | |
| Melanoma* M14 | 7.4 | Gastric ca. KATO III | 25.7 |
| Melanoma* | 7.3 | Colon ca. SW-948 | 3.9 |
| LOXIMVI | | | |
| Melanoma* SK- | 18.0 | Colon ca. SW480 | 34.6 |
| MEL-5 | | | |
| Squamous cell | 5.2 | Colon ca.* (SW480 | 37.4 |
| carcinoma SCC-4 | | met) SW620 | |
| Testis Pool | 6.5 | Colon ca. HT29 | 15.4 |
| Prostate ca.* (bone | 6.1 | Colon ca. HCT-116 | 49.7 |
| met) PC-3 | | | |
| Prostate Pool | 15.6 | Colon ca. CaCo-2 | 51.1 |
| Placenta | 8.8 | Colon cancer tissue | 20.6 |
| Uterus Pool | 6.7 | Colon ca. SW1116 | 6.0 |
| Ovarian ca. OVCAR-3 | 12.2 | Colon ca. Colo-205 | 3.0 |
| Ovarian ca. SK-OV-3 | 46.3 | Colon ca. SW-48 | 2.1 |
| Ovarian ca. OVCAR-4 | 6.0 | Colon Pool | 30.4 |
| Ovarian ca. OVCAR-5 | 27.0 | Small Intestine Pool | 35.1 |
| Ovarian ca. IGROV-1 | 6.3 | Stomach Pool | 20.4 |
| Ovarian ca. OVCAR-8 | 2.8 | Bone Marrow Pool | 15.4 |
| Ovary | 17.9 | Fetal Heart | 17.7 |
| Breast ca. MCF-7 | 0.0 | Heart Pool | 9.3 |
| Breast ca. MDA- | 10.3 | Lymph Node Pool | 40.9 |
| MB-231 | | | |
| Breast ca. BT 549 | 28.3 | Fetal Skeletal Muscle | 15.9 |
| Breast ca. T47D | 28.1 | Skeletal Muscle Pool | 13.8 |
| Breast ca. MDA-N | 4.9 | Spleen Pool | 20.9 |
| Breast Pool | 43.8 | Thymus Pool | 40.1 |
| Trachea | 25.3 | CNS cancer (glio/astro) | 29.3 |
| | | U87-MG | |
| Lung | 9.2 | CNS cancer (glio/astro) | 39.2 |
| | | U-118-MG | |
| Fetal Lung | 50.0 | CNS cancer (neuro;met) | 30.4 |
| | | SK-N-AS | |
| Lung ca. NCI-N417 | 2.0 | CNS cancer (astro) SF- | 5.1 |
| | | 539 | |
| Lung ca. LX-1 | 44.1 | CNS cancer (astro) | 3.4 |
| | | SNB-75 | |
| Lung ca. NCI-H146 | 4.6 | CNS cancer (glio) SNB- | 8.4 |
| | | 19 | |
| Lung ca. SHP-77 | 27.4 | CNS cancer (glio) SF- | 41.5 |
| | | 295 | |
| Lung ca. A549 | 26.8 | Brain (Amygdala) Pool | 5.8 |
| Lung ca. NCI-H526 | 1.7 | Brain (cerebellum) | 20.6 |
| Lung ca. NCI-H23 | 100.0 | Brain (fetal) | 23.7 |
| Lung ca. NCI-H460 | 27.5 | Brain (Hippocampus) | 9.1 |
| | | Pool | |
| Lung ca. HOP-62 | 16.5 | Cerebral Cortex Pool | 12.1 |
| Lung ca. NCI-H522 | 30.4 | Brain (Substantia nigra) | 8.1 |
| | | Pool | |
| Liver | 0.4 | Brain (Thalamus) Pool | 8.5 |
| Fetal Liver | 6.6 | Brain (whole) | 8.9 |
| Liver ca. HepG2 | 10.3 | Spinal Cord Pool | 7.2 |
| Kidney Pool | 40.1 | Adrenal Gland | 5.8 |
| Fetal Kidney | 60.3 | Pituitary gland Pool | 3.9 |
| Renal ca. 786-0 | 0.0 | Salivary Gland | 6.7 |
| Renal ca. A498 | 1.8 | Thyroid (female) | 3.3 |
| Renal ca. ACHN | 25.9 | Pancreatic ca. CAPAN2 | 52.5 |
| Renal ca. UO-31 | 12.5 | Pancreas Pool | 43.8 |

TABLE GD
Panel 4D

| | Rel. Exp.(%) Ag3515, | | Rel. Exp.(%) Ag3515, |
|---|---|---|---|
| Tissue Name | Run 166407127 | Tissue Name | Run 166407127 |
| Secondary Th1 act | 21.3 | HUVEC IL-1 beta | 4.8 |
| Secondary Th2 act | 32.3 | HUVEC IFN gamma | 20.2 |
| Secondary Tr1 act | 46.7 | HUVEC TNF alpha + IFN | 5.8 |
| | | gamma | |
| Secondary Th1 rest | 22.2 | HUVEC TNF alpha + IL4 | 9.5 |
| Secondary Th2 rest | 20.7 | HUVEC IL-11 | 14.7 |
| Secondary Tr1 rest | 28.3 | Lung Microvascular EC | 13.9 |
| | | none | |
| Primary Th1 act | 15.6 | Lung Microvascular EC | 15.4 |
| | | TNF alpha + IL-1 beta | |
| Primary Th2 act | 29.5 | Microvascular Dermal EC | 19.5 |
| | | none | |
| Primary Tr1 act | 38.2 | Microsvasular Dermal EC | 11.7 |
| | | TNF alpha + IL-1 beta | |
| Primary Th1 rest | 100.0 | Bronchial epithelium | 6.5 |
| | | TNF alpha + IL1 beta | |
| Primary Th2 rest | 46.7 | Small airway epithelium | 4.9 |
| | | none | |
| Primary Tr1 rest | 35.4 | Small airway epithelium | 24.3 |
| | | TNF alpha + IL-1 beta | |
| CD45RA CD4 | 13.0 | Coronery artery SMC rest | 4.4 |
| lymphocyte act | | | |
| CD45RO CD4 | 29.9 | Coronery artery SMC | 3.5 |
| lymphocyte act | | TNF alpha + IL-1 beta | |
| CD8 lymphocyte act | 24.3 | Astrocytes rest | 19.6 |
| Secondary CD8 | 23.0 | Astrocytes TNF alpha + IL- | 24.3 |
| lymphocyte rest | | 1 beta | |
| Secondary CD8 | 19.8 | KU-812 (Basophil) rest | 15.2 |
| lymphocyte act | | | |
| CD4 lymphocyte none | 14.7 | KU-812 (Basophil) | 32.8 |
| | | PMA/ionomycin | |
| 2ry Th1/Th2/Tr1_anti- | 41.5 | CCD1106 (Keratinocytes) | 16.4 |
| CD95 CH11 | | none | |
| LAK cells rest | 13.9 | CCD1106 (Keratinocytes) | 62.9 |
| | | TNF alpha + IL-1 beta | |
| LAK cells IL-2 | 32.8 | Liver cirrhosis | 6.4 |
| LAK cells IL-2 + IL-12 | 32.3 | Lupus kidney | 18.0 |
| LAK cells IL-2 + IFN | 49.0 | NCI-H292 none | 35.8 |
| gamma | | | |
| LAK cells IL-2 + IL-18 | 36.1 | NCI-H292 IL-4 | 29.7 |
| LAK cells | 11.7 | NCI-H292 IL-9 | 31.4 |
| PMA/ionomycin | | | |
| NK Cells IL-2 rest | 28.9 | NCI-H292 IL-13 | 26.8 |
| Two Way MLR 3 day | 38.2 | NCI-H292 IFN gamma | 17.2 |
| Two Way MLR 5 day | 12.1 | HPAEC none | 8.3 |
| Two Way MLR 7 day | 15.7 | HPAEC TNF alpha + IL-1 | 17.7 |
| | | beta | |
| PBMC rest | 8.5 | Lung fibroblast none | 22.4 |
| PBMC PWM | 25.2 | Lung fibroblast TNF | 9.9 |
| | | alpha + IL-1 beta | |
| PBMC PHA-L | 10.5 | Lung fibroblast IL-4 | 15.4 |
| Ramos (B cell) none | 51.4 | Lung fibroblast IL-9 | 12.0 |
| Ramos (B cell) | 17.3 | Lung fibroblast IL-13 | 16.7 |
| ionomycin | | | |
| B lymphocytes PWM | 18.6 | Lung fibroblast IFN | 11.3 |
| | | gamma | |
| B lymphocytes CD40L | 49.0 | Dermal fibroblast | 24.5 |
| and IL-4 | | CCD1070 rest | |
| EOL-1 dbcAMP | 28.3 | Dermal fibroblast | 50.3 |
| | | CCD1070 TNF alpha | |
| EOL-1 dbcAMP | 50.7 | Dermal fibroblast | 14.6 |
| PMA/ionomycin | | CCD1070 IL-1 beta | |
| Dendritic cells none | 19.6 | Dermal fibroblast IFN | 10.6 |
| | | gamma | |
| Dendritic cells LPS | 23.0 | Dermal fibroblast IL-4 | 19.8 |
| Dendritic cells anti- | 19.1 | IBD Colitis 2 | 5.8 |
| CD40 | | | |
| Monocytes rest | 47.6 | IBD Crohn's | 3.8 |
| Monocytes LPS | 16.8 | Colon | 61.6 |
| Macrophages rest | 20.7 | Lung | 9.3 |
| Macrophages LPS | 7.1 | Thymus | 32.1 |
| HUVEC none | 12.9 | Kidney | 54.0 |
| HUVEC starved | 22.8 | | |

CNS_neurodegeneration_v1.0 Summary: Ag3515 This panel does not show differential expression of the CG58180-01 gene in Alzheimer's disease. However, this expression profile confirms the presence of this gene in the brain. Please see Panel 1.4 for discussion of utility of this gene in the central nervous system.

General_screening_panel_v1.4 Summary: Ag3515 - Highest expression of the CG58180-01 gene is detected in lung cancer NCI-H23 cell line (CT=28.3). Expression of this gene is associated with gastric, pancreatic, brain, colon, renal, lung, breast, ovarian and prostate cancers, as well as, melanomas. This gene is a homologue of the human prohibitin (PHB), which has been found to be mutated in hereditary breast cancer (Ref. 1). Thus, expression of this gene could be used as a diagnostic marker for the presence of these cancers. Furthermore, therapeutic modulation of the activity of this gene using antibodies or small molecule drugs might be of use in the treatment of these cancers.

In addition, this gene is expressed at significant levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

This gene product is also expressed in adipose, pancreas, adrenal, thyroid, pituitary, skeletal muscle, heart, and liver. This widespread expression in tissues with metabolic function suggests that this gene product may be important for the pathogenesis, diagnosis, and/or treatment of metabolic and endocrine diseases, including obesity and Types 1 and 2 diabetes.

References

1. Sato T, Saito 1-1, Swenseni J, Olifant A, Wood C, Danner D, Sakamoto Tr, Takita K, Kasumi F, Miki Y, et al. The human prohibitin gene located on chromosome 17q21 is mutated in sporadic breast cancer.: Cancer Res Mar. 15, 1992;52(6):1643-6.

Panel 4D Summary: Ag3515 - The CG58180-01 gene is expressed at low to moderate levels in a wide range of cell types of significance in the immune response in health and disease. These cells include members of the T-cell, B-cell, endothelial cell, macrophage/monolytic, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

### H. NOV11a and NOV11b (CG59249-01 and CG59249-02: METALLAPROTEINASE-DISINTEGRIN BETA)

Expression of gene CG59249-01 and CG59249-02 was assessed using the primer-probe set Ag1930, described in Table HA. Results of the RTQ-PCR runs are shown in Table HB.

TABLE HA
Probe Name Ag1930

| | | | | Start |
|---|---|---|---|---|
| Primers | Sequences | | Length | Position |
| Forward | 5′-tatgcagtgtcaagccctttt-3′ | (SEQ ID NO:236) | 21 | 1558 |
| Probe | TET-5′-ctaccaagtgaaagacggttccccag-3′-TAMRA | (SEQ ID NO:237) | 26 | 1582 |
| Reverse | 5′-agtttccaaatcggttaccaat-3′ | (SEQ ID NO:238) | 22 | 1631 |

TABLE HB
Panel 4D

| | Rel. Exp.(%) Ag1930, | | Rel. Exp.(%) Ag1930, |
|---|---|---|---|
| Tissue Name | Run 160658070 | Tissue Name | Run 160658070 |
| Secondary Th1 act | 6.5 | HUVEC IL-1 beta | 1.1 |
| Secondary Th2 act | 4.4 | HUVEC IFN gamma | 11.6 |
| Secondary Tr1 act | 4.6 | HUVEC TNF alpha + IFN | 5.6 |
| | | gamma | |
| Secondary Th1 rest | 4.1 | HUVEC TNF alpha + IL4 | 12.8 |
| Secondary Th2 rest | 4.2 | HUVEC IL-11 | 14.7 |
| Secondary Tr1 rest | 2.0 | Lung Microvascular EC | 14.9 |
| | | none | |
| Primary Th1 act | 3.6 | Lung Microvascular EC | 12.0 |
| | | TNF alpha + IL-1 beta | |
| Primary Th2 act | 1.2 | Microvascular Dermal EC | 20.9 |
| | | none | |
| Primary Tr1 act | 3.5 | Microsvasular Dermal EC | 11.5 |
| | | TNF alpha + IL-1 beta | |
| Primary Th1 rest | 12.1 | Bronchial epithelium | 5.4 |
| | | TNF alpha + IL1 beta | |
| Primary Th2 rest | 13.9 | Small airway epithelium | 31.4 |
| | | none | |
| Primary Tr1 rest | 9.4 | Small airway epithelium | 100.0 |
| | | TNF alpha + IL-1 beta | |
| CD45RA CD4 | 3.6 | Coronery artery SMC rest | 6.1 |
| lymphocyte act | | | |
| CD45RO CD4 | 8.9 | Coronery artery SMC | 10.3 |
| lymphocyte act | | TNF alpha + IL-1 beta | |
| CD8 lymphocyte act | 1.7 | Astrocytes rest | 28.7 |
| Secondary CD8 | 13.5 | Astrocytes TNF alpha + IL- | 21.8 |
| lymphocyte rest | | 1 beta | |
| Secondary CD8 | 3.4 | KU-812 (Basophil) rest | 36.6 |
| lymphocyte act | | | |
| CD4 lymphocyte none | 16.6 | KU-812 (Basophil) | 84.7 |
| | | PMA/ionomycin | |
| 2ry Th1/Th2/Tr1_anti- | 3.6 | CCD1106 (Keratinocytes) | 10.3 |
| CD95 CH11 | | none | |
| LAK cells rest | 11.9 | CCD1106 (Keratinocytes) | 1.6 |
| | | TNF alpha + IL-1 beta | |
| LAK cells IL-2 | 14.4 | Liver cirrhosis | 32.5 |
| LAK cells IL-2 + IL-12 | 17.9 | Lupus kidney | 20.9 |
| LAK cells IL-2 + IFN | 32.3 | NCI-H292 none | 38.7 |
| gamma | | | |
| LAK cells IL-2 + IL-18 | 35.6 | NCI-H292 IL-4 | 16.4 |
| LAK cells | 1.4 | NCI-H292 IL-9 | 53.2 |
| PMA/ionomycin | | | |
| NK Cells IL-2 rest | 5.2 | NCI-H292 IL-13 | 8.2 |
| Two Way MLR 3 day | 27.9 | NCI-H292 IFN gamma | 21.3 |
| Two Way MLR 5 day | 7.7 | HPAEC none | 4.1 |
| Two Way MLR 7 day | 8.2 | HPAEC TNF alpha + IL-1 | 8.2 |
| | | beta | |
| PBMC rest | 7.0 | Lung fibroblast none | 21.3 |
| PBMC PWM | 47.0 | Lung fibroblast TNF | 10.7 |
| | | alpha + IL-1 beta | |
| PBMC PHA-L | 10.3 | Lung fibroblast IL-4 | 17.0 |
| Ramos (B cell) none | 3.1 | Lung fibroblast IL-9 | 24.0 |
| Ramos (B cell) | 20.2 | Lung fibroblast IL-13 | 4.5 |
| ionomycin | | | |
| B lymphocytes PWM | 11.1 | Lung fibroblast IFN | 11.8 |
| | | gamma | |
| B lymphocytes CD40L | 19.2 | Dermal fibroblast | 14.0 |
| and IL-4 | | CCD1070 rest | |
| EOL-1 dbcAMP | 36.9 | Dermal fibroblast | 37.4 |
| | | CCD1070 TNF alpha | |
| EOL-1 dbcAMP | 47.3 | Dermal fibroblast | 5.6 |
| PMA/ionomycin | | CCD1070 IL-1 beta | |
| Dendritic cells none | 11.0 | Dermal fibroblast IFN | 7.1 |
| | | gamma | |
| Dendritic cells LPS | 8.8 | Dermal fibroblast IL-4 | 21.5 |
| Dendritic cells anti- | 27.5 | IBD Colitis 2 | 7.9 |
| CD40 | | | |
| Monocytes rest | 4.0 | IBD Crohn's | 2.6 |
| Monocytes LPS | 3.3 | Colon | 25.5 |
| Macrophages rest | 6.3 | Lung | 4.4 |
| Macrophages LPS | 8.5 | Thymus | 29.7 |
| HUVEC none | 2.6 | Kidney | 100.0 |
| HUVEC starved | 12.2 | | |

Panel 4D Summary: Ag1930 The CG3CG59249-01 gene is widely expressed among the samples in this panel, with highest expression of this gene in the kidney and small airway epithelium treated with TNF-alpha and IL-1 beta (CTs=32). Low but significant levels of expression are also seen in both treated and untreated samples derived from the basophil cell line KU-812, the pulmonary mucoepidermoid cell line NCI-H292, LAK cells and eosinophils. This pattern of expression in a variety of cell types of significance in the immune response in health and disease suggests that this gene product may be involved in homeostatic processes for these and other cell types and tissues. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosis, psoriasis, rheumatoid arthritis, and osteoarthritis.

### I. NOV12a (CG58577-01: CASPR4)

Expression of gene CG58577-01 was assessed using the primer-probe set Ag3377, described in Table IA. Results of the RTQ-PCR runs are shown in Tables IB, IC, ID and IE.

TABLE IA
Probe Name Ag3377

| | | | | Start |
|---|---|---|---|---|
| Primer | Sequences | | Length | Position |
| Forward | 5′-cacagcattatccggagtttt-3′ | (SEQ ID NO:239) | 21 | 2372 |
| Probe | TET-5′-tggcattaaagacttcattcgactcga-3′-TAMRA | (SEQ ID NO:240) | 27 | 2435 |
| Reverse | 5′-ggtgatctctgaaggagagctt-3′ | (SEQ ID NO:241) | 22 | 2435 |

TABLE IB
CNS_neurodegeneration_v1.0

| | Rel. Exp.(%) Ag3377, | | Rel. Exp.(%) Ag3377, |
|---|---|---|---|
| Tissue Name | Run 210154572 | Tissue Name | Run 210154572 |
| AD 1 Hippo | 6.9 | Control (Path) 3 | 2.4 |
| | | Temporal Ctx | |
| AD 2 Hippo | 23.3 | Control (Path) 4 | 41.8 |
| | | Temporal Ctx | |
| AD 3 Hippo | 3.2 | AD 1 Occipital Ctx | 9.9 |
| AD 4 Hippo | 2.7 | AD 2 Occipital Ctx | 0.0 |
| | | (Missing) | |
| AD 5 Hippo | 79.0 | AD 3 Occipital Ctx | 3.7 |
| AD 6 Hippo | 31.0 | AD 4 Occipital Ctx | 11.2 |
| Control 2 Hippo | 28.7 | AD 5 Occipital Ctx | 47.3 |
| Control 4 Hippo | 2.8 | AD 6 Occipital Ctx | 20.3 |
| Control (Path) 3 | 2.7 | Control 1 Occipital | 1.2 |
| Hippo | | Ctx | |
| AD 1 Temporal Ctx | 7.4 | Control 2 Occipital | 92.0 |
| | | Ctx | |
| AD 2 Temporal Ctx | 40.6 | Control 3 Occipital | 14.9 |
| | | Ctx | |
| AD 3 Temporal Ctx | 2.4 | Control 4 Occipital | 2.4 |
| | | Ctx | |
| AD 4 Temporal Ctx | 11.5 | Control (Path) 1 | 100.0 |
| | | Occipital Ctx | |
| AD 5 Inf Temporal | 68.8 | Control (Path) 2 | 14.7 |
| Ctx | | Occipital Ctx | |
| AD 5 Sup Temporal | 20.6 | Control (Path) 3 | 1.2 |
| Ctx | | Occipital Ctx | |
| AD 6 Inf Temporal | 22.4 | Control (Path) 4 | 15.0 |
| Ctx | | Occipital Ctx | |
| AD 6 Sup Temporal | 21.8 | Control 1 Parietal | 3.7 |
| Ctx | | Ctx | |
| Control 1 Temporal | 4.9 | Control 2 Parietal | 18.3 |
| Ctx | | Ctx | |
| Control 2 Temporal | 33.9 | Control 3 Parietal | 11.6 |
| Ctx | | Ctx | |
| Control 3 Temporal | 9.8 | Control (Path) 1 | 87.1 |
| Ctx | | Parietal Ctx | |
| Control 3 Temporal | 26.2 | Control (Path) 2 | 37.4 |
| Ctx | | Parietal Ctx | |
| Control (Path) 1 | 61.1 | Control (Path) 3 | 1.8 |
| Temporal Ctx | | Parietal Ctx | |
| Control (Path) 2 | 26.4 | Control (Path) 4 | 24.8 |
| Temporal Ctx | | Parietal Ctx | |

TABLE IC
Panel 1.3D

| | Rel. Exp.(%) Ag3377, | | Rel. Exp.(%) Ag3377, |
|---|---|---|---|
| Tissue Name | Run 165674254 | Tissue Name | Run 165674254 |
| Liver adenocarcinoma | 0.0 | Kidney (fetal) | 0.0 |
| Pancreas | 0.0 | Renal ca. 786-0 | 0.0 |
| Pancreatic ca. CAPAN2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 2.8 | Renal ca. TK-10 | 0.0 |
| Brain (fetal) | 40.6 | Liver | 0.0 |
| Brain (whole) | 97.9 | Liver (fetal) | 0.0 |
| Brain (amygdala) | 64.2 | Liver ca. | 0.0 |
| | | (hepatoblast) HepG2 | |
| Brain (cerebellum) | 50.7 | Lung | 0.0 |
| Brain (hippocampus) | 72.2 | Lung (fetal) | 0.0 |
| Brain (substantia nigra) | 22.5 | Lung ca. (small cell) | 0.0 |
| | | LX-1 | |
| Brain (thalamus) | 100.0 | Lung ca. (small cell) | 0.0 |
| | | NCI-H69 | |
| Cerebral Cortex | 91.4 | Lung ca. (s.cell var.) | 39.8 |
| | | SHP-77 | |
| Spinal cord | 4.8 | Lung ca. (large | 0.0 |
| | | cell) NCI-H460 | |
| glio/astro U87-MG | 0.0 | Lung ca. (non-sm. | 0.0 |
| | | cell) A549 | |
| glio/astro U-118-MG | 0.0 | Lung ca. (non-s.cell) | 0.0 |
| | | NCI-H23 | |
| astrocytoma SW1783 | 0.0 | Lung ca. (non-s.cell) | 0.0 |
| | | HOP-62 | |
| neuro*; met SK-N-AS | 28.9 | Lung ca. (non-s.cl) | 0.0 |
| | | NCI-H522 | |
| astrocytoma SF-539 | 0.0 | Lung ca. (squam.) | 0.0 |
| | | SW 900 | |
| astrocytoma SNB-75 | 0.0 | Lung ca. (squam.) | 0.0 |
| | | NCI-H596 | |
| glioma SNB-19 | 0.0 | Mammary gland | 1.7 |
| glioma U251 | 0.0 | Breast ca.* (pl.ef) | 0.0 |
| | | MCF-7 | |
| glioma SF-295 | 0.0 | Breast ca.* (pl.ef) | 0.0 |
| | | MDA-MB-231 | |
| Heart (fetal) | 0.0 | Breast ca.* (pl.ef) | 0.0 |
| | | T47D | |
| Heart | 0.0 | Breast ca. BT-549 | 0.0 |
| Skeletal muscle (fetal) | 4.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 1.2 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 0.0 | Ovarian ca.* (ascites) | 0.0 |
| | | SK-OV-3 | |
| Small intestine | 2.0 | Uterus | 0.0 |
| Colon ca. SW480 | 0.0 | Placenta | 0.0 |
| Colon ca.* | 0.0 | Prostate | 0.0 |
| SW620(SW480 met) | | | |
| Colon ca. HT29 | 0.0 | Prostate ca.* (bone | 0.0 |
| | | met) PC-3 | |
| Colon ca. HCT-116 | 0.0 | Testis | 0.0 |
| Colon ca. CaCo-2 | 0.0 | Melanoma | 0.0 |
| | | Hs688(A).T | |
| Colon ca. | 0.0 | Melanoma* (met) | 0.0 |
| tissue (ODO3866) | | Hs688(B).T | |
| Colon ca. HCC-2998 | 0.0 | Melanoma UACC-62 | 0.0 |
| Gastric ca.* (liver met) | 1.8 | Melanoma M14 | 0.0 |
| NCI-N87 | | | |
| Bladder | 0.0 | Melanoma LOX | 0.0 |
| | | IMVI | |
| Trachea | 0.0 | Melanoma* (met) | 0.0 |
| | | SK-MEL-5 | |
| Kidney | 0.0 | Adipose | 0.0 |

TABLE ID
Panel 4D

| | Rel. Exp.(%) Ag3377, | | Rel. Exp.(%) Ag3377, |
|---|---|---|---|
| Tissue Name | Run 165296551 | Tissue Name | Run 165296551 |
| Secondary Th1 act | 0.0 | HUVEC IL-1 beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1 beta | |
| Primary Th2 act | 0.0 | Microvascular Dermal EC | 6.7 |
| | | none | |
| Primary Tr1 act | 0.0 | Microsvasular Dermal EC | 0.0 |
| | | TNF alpha + IL-1 beta | |
| Primary Th1 rest | 0.0 | Bronchial epithelium | 0.0 |
| | | TNF alpha + IL1 beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1 beta | |
| CD45RA CD4 | 0.0 | Coronery artery SMC rest | 100.0 |
| lymphocyte act | | | |
| CD45RO CD4 | 0.0 | Coronery artery SMC | 70.7 |
| lymphocyte act | | TNF alpha + IL-1 beta | |
| CD8 lymphocyte act | 0.0 | Astrocytes rest | 0.0 |
| Secondary CD8 | 0.0 | Astrocytes TNF alpha + IL- | 0.0 |
| lymphocyte rest | | 1 beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1 beta | |
| LAK cells IL-2 | 0.0 | Liver cirrhosis | 0.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF | 0.0 |
| | | alpha + IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1 beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 5.3 |
| CD40 | | | |
| Monocytes rest | 0.0 | IBD Crohn's | 0.0 |
| Monocytes LPS | 0.0 | Colon | 9.0 |
| Macrophages rest | 0.0 | Lung | 0.0 |
| Macrophages LPS | 0.0 | Thymus | 81.8 |
| HUVEC none | 0.0 | Kidney | 47.3 |
| HUVEC starved | 0.0 | | |

TABLE IE
Panel CNS_1

| | Rel. Exp.(%) Ag3377, | | Rel. Exp.(%) Ag3377, |
|---|---|---|---|
| Tissue Name | Run 171787512 | Tissue Name | Run 171787512 |
| BA4 Control | 16.7 | BA17 PSP | 40.3 |
| BA4 Control2 | 67.4 | BA17 PSP2 | 20.0 |
| BA4 | 3.4 | Sub Nigra Control | 38.2 |
| Alzheimer's2 | | | |
| BA4 Parkinson's | 29.1 | Sub Nigra Control2 | 54.7 |
| BA4 | 94.6 | Sub Nigra | 14.5 |
| Parkinson's2 | | Alzheimer's2 | |
| BA4 | 42.3 | Sub Nigra | 34.4 |
| Huntington's | | Parkinson's2 | |
| BA4 | 7.1 | Sub Nigra | 60.3 |
| Huntington's2 | | Huntington's | |
| BA4 PSP | 6.8 | Sub Nigra | 29.1 |
| | | Huntington's2 | |
| BA4 PSP2 | 39.8 | Sub Nigra PSP2 | 3.0 |
| BA4 Depression | 17.3 | Sub Nigra | 4.2 |
| | | Depression | |
| BA4 | 3.2 | Sub Nigra | 8.3 |
| Depression2 | | Depression2 | |
| BA7 Control | 60.3 | Glob Palladus | 1.4 |
| | | Control | |
| BA7 Control2 | 42.0 | Glob Palladus | 3.0 |
| | | Control2 | |
| BA7 | 9.4 | Glob Palladus | 15.5 |
| Alzheimer's2 | | Alzheimer's | |
| BA7 Parkinson's | 21.5 | Glob Palladus | 2.9 |
| | | Alzheimer's2 | |
| BA7 | 80.1 | Glob Palladus | 54.7 |
| Parkinson's2 | | Parkinson's | |
| BA7 | 74.7 | Glob Palladus | 1.7 |
| Huntington's | | Parkinson's2 | |
| BA7 | 42.0 | Glob Palladus PSP | 0.0 |
| Huntington's2 | | | |
| BA7 PSP | 40.1 | Glob Palladus PSP2 | 2.7 |
| BA7 PSP2 | 24.1 | Glob Palladus | 3.1 |
| | | Depression | |
| BA7 Depression | 15.7 | Temp Pole Control | 14.0 |
| BA9 Control | 29.5 | Temp Pole Control2 | 87.1 |
| BA9 Control2 | 100.0 | Temp Pole | 1.2 |
| | | Alzheimer's | |
| BA9 Alzheimer's | 3.0 | Temp Pole | 9.7 |
| | | Alzheimer's2 | |
| BA9 | 21.6 | Temp Pole | 25.7 |
| Alzheimer's2 | | Parkinson's | |
| BA9 Parkinson's | 39.5 | Temp Pole | 21.9 |
| | | Parkinson's2 | |
| BA9 | 74.7 | Temp Pole | 48.0 |
| Parkinson's2 | | Huntington's | |
| BA9 | 62.0 | Temp Pole PSP | 1.7 |
| Huntington's | | | |
| BA9 | 2.6 | Temp Pole PSP2 | 3.6 |
| Huntington's2 | | | |
| BA9 PSP | 10.8 | Temp Pole | 10.3 |
| | | Depression2 | |
| BA9 PSP2 | 1.6 | Cing Gyr Control | 99.3 |
| BA9 Depression | 8.2 | Cing Gyr Control2 | 57.4 |
| BA9 | 9.0 | Cing Gyr Alzheimer's | 18.6 |
| Depression2 | | | |
| BA17 Control | 88.9 | Cing Gyr | 4.3 |
| | | Alzheimer's2 | |
| BA17 Control2 | 84.1 | Cing Gyr Parkinson's | 11.0 |
| BA17 | 6.1 | Cing Gyr | 24.5 |
| Alzheimer's2 | | Parkinson's2 | |
| BA17 | 56.3 | Cing Gyr | 62.9 |
| Parkinson's | | Huntington's | |
| BA17 | 80.1 | Cing Gyr | 11.1 |
| Parkinson's2 | | Huntington's2 | |
| BA17 | 89.5 | Cing Gyr PSP | 17.1 |
| Huntington's | | | |
| BA17 | 25.2 | Cing Gyr PSP2 | 1.4 |
| Huntington's2 | | | |
| BA17 | 7.6 | Cing Gyr Depression | 4.3 |
| Depression | | | |
| BA17 | 18.7 | Cing Gyr | 9.8 |
| Depression2 | | Depression2 | |

CNS_neurodegeneration_v1.0 Summary: Ag3377 This panel confirms the expression of the CG58577-01 gene at low levels in the brain in an independent group of individuals. However, no differential expression of this gene was detected between Alzheimer's diseased postmortem brains and those of non-demented controls in this experiment. Please see Panel 1.3D for a discussion of the potential utility of this gene in treatment of central nervous system disorders.

Panel 1.3D Summary: Ag3377 Expression of the CG58577-01 gene is seen almost exclusively in the brain. The highest level of expression is in the thalamus (CT=3 1.0), with low to moderate expression in amygdala, substanitia nigra, cerebellum, hippocampus and cerebral cortex. This gene encodes a protein homologous to the mouse Caspr4 protein. The Caspr proteins are contactin-associated transmembrane receptors that may function with contactin, in the recruitment and activation of neural intracellular signaling pathways. Therefore, this gene may play a role in central nervous system disorders such as Alzheimer's disease, Parkinson's disease, epilepsy, multiple sclerosis, schizophrenia and depression.

There is also low levels of expression in lung cancer cell line SHP-77 and neuroblastoma cancer cell line SK-N-AS (CTs=32). Therefore, therapeutic modulation of the activity of this gene or its protein product, through the use of small molecule drugs, protein therapeutics or antibodies, might be beneficial in the treatment of lung cancer or neuroblastoma cancer.

References

1. Peles E, Nativ M, Lustig M, Grumet M, Schilling J, Martinez R, Plowman G D, Schlessinger J. (1997) Identification of a novel contactin-associated transmembrane receptor with multiple domains implicated in protein-protein interactions. EMBO J 16(5):978-88.

Panel 4D Summary: Ag3377 Significant expression of the CG58577-01 gene is seen only in coronary artery, thymus, and kidney (CTs=33-34.8). Therefore expression of this gene can be used to distinguish between these samples and others on this panel. In addition, therapeutic modulation of the activity of the CASPR protein encoded by this gene may be useful in the treatment of asthma, restenosis, arthritis, systemic lupus erythematosus and kidney disorders.

Panel CNS_{—}1 Summary: Ag3377—This panel confirms the expression of this gene at significant levels in the brain in an independent group of individuals. Please see Panel 1.4 for a discussion of the potential utility of this gene in treatment of central nervous system disorders.

### J. NOV14 (CG58575-01: PHOSPHATIDYLSERINE SYNTHASE-2)

Expression of gene CG58575-01 was assessed using the primer-probe set Ag3376, described in Table JA. Results of the RTQ-PCR runs are shown in Tables JB, JC, JD and JE.

TABLE JA
Probe Name Ag3376

| | | | | Start |
|---|---|---|---|---|
| Primer | Sequences | | Length | Position |
| Forward | 5′-atctacgacccagacaatgaga-3′ | (SEQ ID NO:242) | 22 | 592 |
| Probe | TET-5′-cccctttcacaacatctgggacaagt-3′-TAMRA | (SEQ ID NO:243) | 26 | 618 |
| Reverse | 5′-tcaggtaccagccaagaaagt-3′ | (SEQ ID NO:244) | 21 | 665 |

TABLE JB
CNS_neurodegeneration_v1.0

| | Rel. Exp.(%) Ag3376, | | Rel. Exp.(%) Ag3376, |
|---|---|---|---|
| Tissue Name | Run 210154240 | Tissue Name | Run 210154240 |
| AD 1 Hippo | 18.8 | Control (Path) 3 | 6.1 |
| | | Temporal Ctx | |
| AD 2 Hippo | 48.3 | Control (Path) 4 | 18.8 |
| | | Temporal Ctx | |
| AD 3 Hippo | 7.6 | AD 1 Occipital Ctx | 14.5 |
| AD 4 Hippo | 11.2 | AD 2 Occipital Ctx | 0.0 |
| | | (Missing) | |
| AD 5 hippo | 60.7 | AD 3 Occipital Ctx | 6.8 |
| AD 6 Hippo | 38.7 | AD 4 Occipital Ctx | 17.4 |
| Control 2 Hippo | 24.1 | AD 5 Occipital Ctx | 17.7 |
| Control 4 Hippo | 13.6 | AD 6 Occipital Ctx | 34.9 |
| Control (Path) 3 | 9.4 | Control 1 Occipital | 3.7 |
| Hippo | | Ctx | |
| AD 1 Temporal Ctx | 20.0 | Control 2 Occipital | 52.5 |
| | | Ctx | |
| AD 2 Temporal Ctx | 35.6 | Control 3 Occipital | 14.8 |
| | | Ctx | |
| AD 3 Temporal Ctx | 8.4 | Control 4 Occipital | 8.0 |
| | | Ctx | |
| AD 4 Temporal Ctx | 18.4 | Control (Path) 1 | 57.4 |
| | | Occipital Ctx | |
| AD 5 Inf Temporal | 100.0 | Control (Path) 2 | 9.0 |
| Ctx | | Occipital Ctx | |
| AD 5 Sup Temporal | 59.9 | Control (Path) 3 | 7.2 |
| Ctx | | Occipital Ctx | |
| AD 6 Inf Temporal | 39.2 | Control (Path) 4 | 17.4 |
| Ctx | | Occipital Ctx | |
| AD 6 Sup Temporal | 40.1 | Control 1 Parietal | 7.9 |
| Ctx | | Ctx | |
| Control 1 Temporal | 8.1 | Control 2 Parietal | 47.3 |
| Ctx | | Ctx | |
| Control 2 Temporal | 31.2 | Control 3 Parietal | 19.1 |
| Ctx | | Ctx | |
| Control 3 Temporal | 14.8 | Control (Path) 1 | 39.0 |
| Ctx | | Parietal Ctx | |
| Control 4 Temporal | 14.3 | Control (Path) 2 | 17.4 |
| Ctx | | Parietal Ctx | |
| Control (Path) 1 | 51.1 | Control (Path) 3 | 4.5 |
| Temporal Ctx | | Parietal Ctx | |
| Control (Path) 2 | 19.9 | Control (Path) 4 | 26.8 |
| Temporal Ctx | | Parietal Ctx | |

TABLE JC
Panel 1.3D

| | Rel. Exp.(%) Ag3376, | | Rel. Exp.(%) Ag3376, |
|---|---|---|---|
| Tissue Name | Run 165674234 | Tissue Name | Run 165674234 |
| Liver adenocarcinoma | 19.1 | Kidney (fetal) | 13.8 |
| Pancreas | 7.5 | Renal ca. 786-0 | 6.5 |
| Pancreatic ca. CAPAN2 | 6.5 | Renal ca. A498 | 12.4 |
| Adrenal gland | 13.5 | Renal ca. RXF 393 | 21.9 |
| Thyroid | 11.7 | Renal ca. ACHN | 1.7 |
| Salivary gland | 10.2 | Renal ca. UO-31 | 8.2 |
| Pituitary gland | 8.1 | Renal ca. TK-10 | 8.0 |
| Brain (fetal) | 31.9 | Liver | 2.0 |
| Brain (whole) | 82.9 | Liver (fetal) | 7.0 |
| Brain (amygdala) | 50.3 | Liver ca. | 11.3 |
| | | (hepatoblast) HepG2 | |
| Brain (cerebellum) | 57.0 | Lung | 8.4 |
| Brain (hippocampus) | 51.4 | Lung (fetal) | 3.6 |
| Brain (substantia nigra) | 30.4 | Lung ca. (small cell) | 8.4 |
| | | LX-1 | |
| Brain (thalamus) | 51.8 | Lung ca. (small cell) | 0.7 |
| | | NCI-H69 | |
| Cerebral Cortex | 27.5 | Lung ca. (s.cell var.) | 5.4 |
| | | SHP-77 | |
| Spinal cord | 31.6 | Lung ca. (large | 21.5 |
| | | cell) NCI-H460 | |
| glio/astro U87-MG | 6.3 | Lung ca. (non-sm. | 4.2 |
| | | cell) A549 | |
| glio/astro U-118-MG | 7.7 | Lung ca. (non-s.cell) | 5.8 |
| | | NCI-H23 | |
| astrocytoma SW1783 | 2.3 | Lung ca. (non-s.cell) | 9.2 |
| | | HOP-62 | |
| neuro*; met SK-N-AS | 6.6 | Lung ca. (non-s.cl) | 10.5 |
| | | NCI-H522 | |
| astrocytoma SF-539 | 1.7 | Lung ca. (squam.) | 1.8 |
| | | SW 900 | |
| astrocytoma SNB-75 | 4.7 | Lung ca. (squam.) | 9.7 |
| | | NCI-H596 | |
| glioma SNB-19 | 5.0 | Mammary gland | 15.7 |
| glioma U251 | 17.3 | Breast ca.* (pl.ef) | 2.0 |
| | | MCF-7 | |
| glioma SF-295 | 4.2 | Breast ca.* (pl.ef) | 53.2 |
| | | MDA-MB-231 | |
| Heart (fetal) | 29.1 | Breast ca.* (pl.ef) | 1.9 |
| | | T47D | |
| Heart | 11.6 | Breast ca. BT-549 | 34.2 |
| Skeletal muscle (fetal) | 13.1 | Breast ca. MDA-N | 1.1 |
| Skeletal muscle | 44.4 | Ovary | 10.8 |
| Bone marrow | 4.5 | Ovarian ca. OVCAR-3 | 6.5 |
| Thymus | 6.6 | Ovarian ca. OVCAR-4 | 7.9 |
| Spleen | 12.6 | Ovarian ca. OVCAR-5 | 5.2 |
| Lymph node | 14.5 | Ovarian ca. OVCAR-8 | 3.9 |
| Colorectal | 2.1 | Ovarian ca. IGROV-1 | 2.5 |
| Stomach | 13.7 | Ovarian ca.* (ascites) | 5.2 |
| | | SK-OV-3 | |
| Small intestine | 14.8 | Uterus | 35.4 |
| Colon ca. SW480 | 9.2 | Placenta | 11.6 |
| Colon ca.* | 4.9 | Prostate | 16.0 |
| SW620(SW480 met) | | | |
| Colon ca. HT29 | 3.4 | Prostate ca.* (bone | 11.0 |
| | | met) PC-3 | |
| Colon ca. HCT-116 | 8.3 | Testis | 100.0 |
| Colon ca. CaCo-2 | 5.1 | Melanoma | 3.6 |
| | | Hs688(A).T | |
| Colon ca. | 8.5 | Melanoma* (met) | 4.1 |
| tissue (ODO3866) | | Hs688(B).T | |
| Colon ca. HCC-2998 | 4.9 | Melanoma UACC-62 | 20.4 |
| Gastric ca.* (liver met) | 11.9 | Melanoma M14 | 10.2 |
| NCI-N87 | | | |
| Bladder | 1.9 | Melanoma LOX | 5.2 |
| | | IMVI | |
| Trachea | 7.2 | Melanoma* (met) | 4.0 |
| | | SK-MEL-5 | |
| Kidney | 14.3 | Adipose | 2.5 |

TABLE JD
Panel 4D

| | Rel. Exp.(%) Ag3376, | | Rel. Exp.(%) Ag3376, |
|---|---|---|---|
| Tissue Name | Run 165296548 | Tissue Name | Run 165296548 |
| Secondary Th1 act | 41.2 | HUVEC IL-1 beta | 6.5 |
| Secondary Th2 act | 26.2 | HUVEC IFN gamma | 38.2 |
| Secondary Tr1 act | 36.3 | HUVEC TNF alpha + IFN | 18.4 |
| | | gamma | |
| Secondary Th1 rest | 4.4 | HUVEC TNF alpha + IL4 | 22.5 |
| Secondary Th2 rest | 6.8 | HUVEC IL-11 | 30.6 |
| Secondary Tr1 rest | 11.6 | Lung Microvascular EC | 32.1 |
| | | none | |
| Primary Th1 act | 15.5 | Lung Microvascular EC | 32.8 |
| | | TNF alpha + IL-1 beta | |
| Primary Th2 act | 21.2 | Microvascular Dermal EC | 40.6 |
| | | none | |
| Primary Tr1 act | 45.1 | Microsvasular Dermal EC | 28.7 |
| | | TNF alpha + IL-1 beta | |
| Primary Th1 rest | 20.4 | Bronchial epithelium | 62.9 |
| | | TNF alpha + IL1 beta | |
| Primary Th2 rest | 7.9 | Small airway epithelium | 19.6 |
| | | none | |
| Primary Tr1 rest | 9.3 | Small airway epithelium | 55.9 |
| | | TNF alpha + IL-1 beta | |
| CD45RA CD4 | 13.4 | Coronery artery SMC rest | 34.4 |
| lymphocyte act | | | |
| CD45RO CD4 | 8.1 | Coronery artery SMC | 29.7 |
| lymphocyte act | | TNF alpha + IL-1 beta | |
| CD8 lymphocyte act | 12.5 | Astrocytes rest | 28.9 |
| Secondary CD8 | 12.9 | Astrocytes TNF alpha + IL- | 22.4 |
| lymphocyte rest | | 1 beta | |
| Secondary CD8 | 6.7 | KU-812 (Basophil) rest | 33.7 |
| lymphocyte act | | | |
| CD4 lymphocyte none | 2.5 | KU-812 (Basophil) | 55.1 |
| | | PMA/ionomycin | |
| 2ry Th1/Th2/Tr1_anti- | 6.8 | CCD1106 (Keratinocytes) | 31.6 |
| CD95 CH11 | | none | |
| LAK cells rest | 6.6 | CCD1106 (Keratinocytes) | 22.5 |
| | | TNF alpha + IL-1 beta | |
| LAK cells IL-2 | 9.6 | Liver cirrhosis | 1.7 |
| LAK cells IL-2 + IL-12 | 14.1 | Lupus kidney | 3.2 |
| LAK cells IL-2 + IFN | 12.9 | NCI-H292 none | 40.3 |
| gamma | | | |
| LAK cells IL-2 + IL-18 | 8.7 | NCI-H292 IL-4 | 67.8 |
| LAK cells | 3.4 | NCI-H292 IL-9 | 70.2 |
| PMA/ionomycin | | | |
| NK Cells IL-2 rest | 4.8 | NCI-H292 IL-13 | 48.3 |
| Two Way MLR 3 day | 9.1 | NCI-H292 IFN gamma | 42.3 |
| Two Way MLR 5 day | 14.5 | HPAEC none | 37.9 |
| Two Way MLR 7 day | 8.4 | HPAEC TNF alpha + IL-1 | 23.0 |
| | | beta | |
| PBMC rest | 3.3 | Lung fibroblast none | 32.1 |
| PBMC PWM | 46.3 | Lung fibroblast TNF alpha | 19.2 |
| | | + IL-1 beta | |
| PBMC PHA-L | 12.0 | Lung fibroblast IL-4 | 76.3 |
| Ramos (B cell) none | 24.5 | Lung fibroblast IL-9 | 49.7 |
| Ramos (B cell) | 100.0 | Lung fibroblast IL-13 | 33.2 |
| ionomycin | | | |
| B lymphocytes PWM | 57.8 | Lung fibroblast IFN | 79.6 |
| | | gamma | |
| B lymphocytes CD40L | 12.5 | Dermal fibroblast | 29.7 |
| and IL-4 | | CCD1070 rest | |
| EOL-1 dbcAMP | 17.1 | Dermal fibroblast | 39.5 |
| | | CCD1070 TNF alpha | |
| EOL-1 dbcAMP | 6.7 | Dermal fibroblast | 21.0 |
| PMA/ionomycin | | CCD1070 IL-1 beta | |
| Dendritic cells none | 7.5 | Dermal fibroblast IFN | 16.0 |
| | | gamma | |
| Dendritic cells LPS | 7.7 | Dermal fibroblast IL-4 | 28.7 |
| Dendritic cells anti- | 5.4 | IBD Colitis 2 | 0.3 |
| CD40 | | | |
| Monocytes rest | 12.5 | IBD Crohn's | 1.1 |
| Monocytes LPS | 3.4 | Colon | 11.2 |
| Macrophages rest | 23.0 | Lung | 14.6 |
| Macrophages LPS | 3.3 | Thymus | 59.9 |
| HUVEC none | 46.3 | Kidney | 26.8 |
| HUVEC starved | 34.9 | | |

TABLE JE
Panel CNS_1

| | Rel. Exp.(%) Ag3376, | | Rel. Exp.(%) Ag3376, |
|---|---|---|---|
| Tissue Name | Run 171787488 | Tissue Name | Run 171787488 |
| BA4 Control | 29.5 | BA17 PSP | 22.4 |
| BA4 Control2 | 49.0 | BA17 PSP2 | 11.3 |
| BA4 | 3.5 | Sub Nigra Control | 66.9 |
| Alzheimer's2 | | | |
| BA4 Parkinson's | 52.9 | Sub Nigra Control2 | 27.4 |
| BA4 | 71.2 | Sub Nigra | 11.0 |
| Parkinson's2 | | Alzheimer's2 | |
| BA4 | 44.4 | Sub Nigra | 100.0 |
| Huntington's | | Parkinson's2 | |
| BA4 | 24.3 | Sub Nigra | 77.4 |
| Huntington's2 | | Huntington's | |
| BA4 PSP | 11.0 | Sub Nigra | 53.2 |
| | | Huntington's2 | |
| BA4 PSP2 | 20.6 | Sub Nigra PSP2 | 8.7 |
| BA4 Depression | 15.6 | Sub Nigra | 9.9 |
| | | Depression | |
| BA4 | 6.7 | Sub Nigra | 9.1 |
| Depression2 | | Depression2 | |
| BA7 Control | 42.3 | Glob Palladus | 34.4 |
| | | Control | |
| BA7 Control2 | 44.8 | Glob Palladus | 29.9 |
| | | Control2 | |
| BA7 | 4.5 | Glob Palladus | 26.4 |
| Alzheimer's2 | | Alzheimer's | |
| BA7 Parkinson's | 33.9 | Glob Palladus | 1.9 |
| | | Alzheimer's2 | |
| BA7 | 22.1 | Glob Palladus | 95.3 |
| Parkinson's2 | | Parkinson's | |
| BA7 | 40.1 | Glob Palladus | 26.6 |
| Huntington's | | Parkinson's2 | |
| BA7 | 79.6 | Glob Palladus PSP | 5.8 |
| Huntington's2 | | | |
| BA7 PSP | 22.4 | Glob Palladus PSP2 | 6.4 |
| BA7 PSP2 | 25.5 | Glob Palladus | 0.0 |
| | | Depression | |
| BA7 Depression | 19.1 | Temp Pole Control | 15.6 |
| BA9 Control | 24.0 | Temp Pole Control2 | 35.8 |
| BA9 Control2 | 92.7 | Temp Pole | 6.7 |
| | | Alzheimer's | |
| BA9 Alzheimer's | 9.9 | Temp Pole | 5.1 |
| | | Alzheimer's2 | |
| BA9 | 13.7 | Temp Pole | 37.1 |
| Alzheimer's2 | | Parkinson's | |
| BA9 Parkinson's | 43.8 | Temp Pole | 36.9 |
| | | Parkinson's2 | |
| BA9 | 58.6 | Temp Pole | 53.6 |
| Parkinson's2 | | Huntington's | |
| BA9 | 49.7 | Temp Pole PSP | 1.4 |
| Huntington's | | | |
| BA9 | 19.5 | Temp Pole PSP2 | 3.5 |
| Huntington's2 | | | |
| BA9 PSP | 14.1 | Temp Pole | 7.5 |
| | | Depression2 | |
| BA9 PSP2 | 2.0 | Cing Gyr Control | 80.7 |
| BA9 Depression | 11.3 | Cing Gyr Control2 | 54.0 |
| BA9 | 22.5 | Cing Gyr Alzheimer's | 24.8 |
| Depression2 | | | |
| BA17 Control | 65.1 | Cing Gyr | 6.8 |
| | | Alzheimer's2 | |
| BA17 Control2 | 67.4 | Cing Gyr Parkinson's | 70.2 |
| BA17 | 8.2 | Cing Gyr | 47.3 |
| Alzheimer's2 | | Parkinson's2 | |
| BA17 | 57.0 | Cing Gyr | 72.2 |
| Parkinson's | | Huntington's | |
| BA17 | 46.7 | Cing Gyr | 23.5 |
| Parkinson's2 | | Huntington's2 | |
| BA17 | 86.5 | Cing Gyr PSP | 12.1 |
| Huntington's | | | |
| BA17 | 19.3 | Cing Gyr PSP2 | 3.8 |
| Huntington's2 | | | |
| BA17 | 26.2 | Cing Gyr Depression | 11.4 |
| Depression | | | |
| BA17 | 42.0 | Cing Gyr | 11.8 |
| Depression2 | | Depression2 | |

CNS_neurodegeneration_v1.0 Summary: Ag3376 This panel does not show differential expression of the CG58575-01 gene in Alzheimer's disease. However, this expression profile confirms the presence of this gene in the brain. Please see Panel 1.3D for discussion of utility of this gene in the central nervous system.

Panel 1.3D Summary: Ag3376 The CG58575-01 gene encodes a putative phosphatidylserine synthase-2. In agreement with published results on the mouse homologue of this gene, the highest expression of this gene is in testis (CT=29.5).

Expression of this gene is confirmed in all regions of the brain. Phosphatidylserine synthase activity was found to be reduced in the mnd/mnd mouse, a model for the human degenerative disease neuronal ceroid lipofuscinosis. Therefore, this gene may play a role in neurodegenerative disorders in which there is an abnormal accumulation of lipids and proteins in cellular storage bodies.

In addition, expression of this gene is higher in breast cancer cell lines when compared to expression in normal breast tissue. Thus, expression of this gene could be used to differentiate between breast cancer cell lines and other samples on this panel and as a marker for breast cancer. Furthermore, therapeutic modulation of the expression or function of this gene may be effective in the treatment of breast cancer.

Among tissues with metabolic or endocrine function, this gene is expressed at low to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

References

1. Vance J E, Stone S J, Faust J R. Abnormalities in mitochondria-associated membranes and phospholipid biosynthetic enzymes in the mnd/mnd mouse model of neuronal ceroid lipofuscinosis.Biochim Biophys Acta Feb. 18, 1997; 1344(3):286-99.

2. Sturbois-Balcerzak B, Stone S J, Sreenivas A, Vance J E. Structure and expression of the murine phosphatidylserine syntilase-1 gene. J Biol Chem Mar. 16, 2001; 276(11):8205-12.

Panel 4D Summary: Ag3376 Highest expression of the CG58575-01 gene is seen in the B cell line Ramos treated with ionomycin (CT=29.3). Significant levels of expression are seen in activated and untreated NCI-H292 cells, IL-4, 1L-9, IL-13 and IFN gamma activated lung fibroblasts, human pulmonary aortic endothelial cells (treated and untreated), small airway epithelium (treated and untreated), treated bronchial epithelium and lung microvascular endothelial cells (treated and untreated). The expression of this gene in cells derived from or within the lung suggests that this gene may be involved in normal conditions as well as pathological and inflammatory lung disorders that include chronic obstructive pulmonary disease, asthma, allergy and emphysema. Moderate/low expression of this gene is also detected in treated and untreated HUVECs (endothelial cells), coronary artery smooth muscle cells (treated and untreated), treated and untreated astrocytes, treated KU-812 basophils, treated and untreated CCD1106 keratinocytes, IL-4 treated dermal fibroblasts, and normal tissues that include lung, colon, thymus and kidney. Expression in the various immune cell types (as well as in diseased tissue samples) suggests that therapeutic modulation of this gene product may ameliorate symptoms associated with infectious conditions as well as inflammatory and autoimmune disorders that include psoriasis, allergy, asthma, inflammatory bowel disease, rheumatoid arthritis and osteoarthritis.

Panel CNS_{—}1 Summary: AG3376 This panel confirms expression of the CG58575-01 gene in the brain. Please see Panel 1.3D for discussion of utility of this gene in the central nervous system.

### K. NOV16a (CG59239-01: MHC CLASS I)

Expression of gene CG59239-01 was assessed using the primer-probe sets Ag3516 and Ag3517, described in Tables KA and KB. Results of the RTQ-PCR runs are shown in Tables KC, KD and KE.

TABLE KA
Probe Name Ag3516

| | | | | Start |
|---|---|---|---|---|
| Primer | Sequences | | Length | Position |
| Forward | 5′-cggagaggagcagagataca-3′ | (SEQ ID NO:245) | 20 | 845 |
| Probe | TET-5′-cagagcccctcaccctgagatgg-3′-TAMRA | (SEQ ID NO:246) | 23 | 892 |
| Reverse | 5′-gatggtgggctgagaagac-3′ | (SEQ ID NO:247) | 19 | 920 |

TABLE KB
Probe Name Ag3517

| | | | | Start |
|---|---|---|---|---|
| Primers | Sequenecs | | Length | Position |
| Forward | 5′-cggagaggagcagagataca-3′ | (SEQ ID NO:248) | 20 | 845 |
| Probe | TET-5′-cagagcccctcaccctgagatgg-3′-TAMRA | (SEQ ID NO:249) | 23 | 892 |
| Reverse | 5′-gatggtgggctgagaagac-3′ | (SEQ ID NO:250) | 19 | 920 |

TABLE KC
CNS_neurodegeneration_v1.0

| | Rel. Exp.(%) | Rel. Exp.(%) | | Rel. Exp.(%) | Rel. Exp.(%) |
|---|---|---|---|---|---|
| | Ag3516, Run | Ag3517, Run | | Ag3516, Run | Ag3517, Run |
| Tissue Name | 211005124 | 210629732 | Tissue Name | 211005124 | 210629732 |
| AD 1 Hippo | 48.6 | 34.2 | Control | 20.7 | 23.0 |
| | | | (Path) 3 | | |
| | | | Temporal | | |
| | | | Ctx | | |
| AD 2 Hippo | 87.7 | 88.3 | Control | 32.8 | 28.3 |
| | | | (Path) 4 | | |
| | | | Temporal | | |
| | | | Ctx | | |
| AD 3 Hippo | 10.9 | 10.7 | AD 1 | 25.3 | 25.3 |
| | | | Occipital Ctx | | |
| AD 4 Hippo | 13.4 | 11.3 | AD 2 | 0.0 | 0.0 |
| | | | Occipital Ctx | | |
| | | | (Missing) | | |
| AD 5 Hippo | 79.6 | 85.9 | AD 3 | 9.8 | 10.3 |
| | | | Occipital Ctx | | |
| AD 6 Hippo | 59.5 | 57.0 | AD 4 | 16.6 | 17.3 |
| | | | Occipital Ctx | | |
| Control 2 | 63.7 | 71.2 | AD 5 | 73.2 | 87.7 |
| Hippo | | | Occipital Ctx | | |
| Control 4 | 33.9 | 24.7 | AD 6 | 2.8 | 18.0 |
| Hippo | | | Occipital Ctx | | |
| Control | 11.1 | 11.3 | Control 1 | 6.3 | 6.0 |
| (Path) 3 | | | Occipital Ctx | | |
| Hippo | | | | | |
| AD 1 | 41.8 | 40.6 | Control 2 | 100.0 | 100.0 |
| Temporal | | | Occipital Ctx | | |
| Ctx | | | | | |
| AD 2 | 52.9 | 78.5 | Control 3 | 14.4 | 11.7 |
| Temporal | | | Occipital Ctx | | |
| Ctx | | | | | |
| AD 3 | 9.7 | 8.4 | Control 4 | 12.8 | 12.2 |
| Temporal | | | Occipital Ctx | | |
| Ctx | | | | | |
| AD 4 | 24.1 | 23.2 | Control | 85.3 | 92.7 |
| Temporal | | | (Path) 1 | | |
| Ctx | | | Occipital Ctx | | |
| AD 5 Inf | 87.1 | 87.7 | Control | 22.7 | 16.7 |
| Temporal | | | (Path) 2 | | |
| Ctx | | | Occipital Ctx | | |
| AD 5 Sup | 79.0 | 85.9 | Control | 21.8 | 23.3 |
| Temporal | | | (Path) 3 | | |
| Ctx | | | Occipital Ctx | | |
| AD 6 Inf | 48.3 | 52.5 | Control | 29.3 | 27.4 |
| Temporal | | | (Path) 4 | | |
| Ctx | | | Occipital Ctx | | |
| AD 6 Sup | 46.3 | 50.3 | Control 1 | 7.8 | 8.6 |
| Temporal | | | Parietal Ctx | | |
| Ctx | | | | | |
| Control 1 | 6.8 | 6.1 | Control 2 | 59.9 | 70.2 |
| Temporal | | | Parietal Ctx | | |
| Ctx | | | | | |
| Control 2 | 61.6 | 79.6 | Control 3 | 21.9 | 16.3 |
| Temporal | | | Parietal Ctx | | |
| Ctx | | | | | |
| Control 3 | 16.4 | 15.0 | Control | 69.3 | 84.1 |
| Temporal | | | (Path) 1 | | |
| Ctx | | | Parietal Ctx | | |
| Control 3 | 16.2 | 14.0 | Control | 47.3 | 51.1 |
| Temporal | | | (Path) 2 | | |
| Ctx | | | Parietal Ctx | | |
| Control | 75.8 | 82.4 | Control | 20.2 | 23.5 |
| (Path) 1 | | | (Path) 3 | | |
| Temporal | | | Parietal Ctx | | |
| Ctx | | | | | |
| Control | 49.7 | 45.7 | Control | 40.6 | 43.8 |
| (Path) 2 | | | (Path) 4 | | |
| Temporal | | | Parietal Ctx | | |
| Ctx | | | | | |

TABLE KD
General_screening_panel_v1.4

| | Rel. Exp.(%) | Rel. Exp.(%) | | Rel. Exp.(%) | Rel. Exp.(%) |
|---|---|---|---|---|---|
| | Ag3516, Run | Ag3517, Run | | Ag3516, Run | Ag3517, Run |
| Tissue Name | 216861071 | 216862935 | Tissue Name | 216861071 | 216862935 |
| Adipose | 5.2 | 5.7 | Renal ca. TK-10 | 6.4 | 9.5 |
| Melanoma* | 7.1 | 8.4 | Bladder | 15.4 | 18.2 |
| Hs688(A).T | | | | | |
| Melanoma* | 5.7 | 6.0 | Gastric ca. (liver | 13.2 | 15.4 |
| Hs688(B).T | | | met.) NCI-N87 | | |
| Melanoma* | 6.0 | 8.5 | Gastric ca. KATO | 62.9 | 52.9 |
| M14 | | | III | | |
| Melanoma* | 1.5 | 1.5 | Colon ca. SW- | 3.4 | 3.3 |
| LOXIMVI | | | 948 | | |
| Melanoma* | 7.3 | 8.7 | Colon ca. SW480 | 5.5 | 5.6 |
| SK-MEL-5 | | | | | |
| Squamous cell | 3.2 | 3.0 | Colon ca.* | 5.2 | 6.4 |
| carcinoma | | | (SW480 met) | | |
| SCC-4 | | | SW620 | | |
| Testis Pool | 1.4 | 1.3 | Colon ca. HT29 | 4.8 | 5.3 |
| Prostate ca.* | 3.5 | 3.0 | Colon ca. HCT- | 3.7 | 5.7 |
| (bone met) | | | 116 | | |
| PC-3 | | | | | |
| Prostate Pool | 3.5 | 2.7 | Colon ca. CaCo-2 | 7.2 | 9.7 |
| Placenta | 2.1 | 2.3 | Colon cancer | 30.6 | 54.0 |
| | | | tissue | | |
| Uterus Pool | 2.2 | 1.9 | Colon ca. | 0.0 | 0.0 |
| | | | SW1116 | | |
| Ovarian ca. | 1.6 | 1.7 | Colon ca. Colo- | 6.2 | 5.5 |
| OVCAR-3 | | | 205 | | |
| Ovarian ca. | 66.4 | 62.9 | Colon ca. SW-48 | 18.8 | 19.9 |
| SK-OV-3 | | | | | |
| Ovarian ca. | 1.7 | 2.0 | Colon Pool | 3.9 | 4.9 |
| OVCAR-4 | | | | | |
| Ovarian ca. | 18.7 | 20.2 | Small Intestine | 9.1 | 10.0 |
| OVCAR-5 | | | Pool | | |
| Ovarian ca. | 32.3 | 63.3 | Stomach Pool | 2.9 | 1.7 |
| IGROV-1 | | | | | |
| Ovarian ca. | 81.8 | 73.7 | Bone Marrow | 1.1 | 0.8 |
| OVCAR-8 | | | Pool | | |
| Ovary | 5.3 | 6.2 | Fetal Heart | 0.9 | 1.1 |
| Breast ca. | 2.0 | 1.5 | Heart Pool | 2.9 | 3.1 |
| MCF-7 | | | | | |
| Breast ca. | 32.3 | 34.6 | Lymph Node | 2.9 | 2.7 |
| MDA-MB- | | | Pool | | |
| 231 | | | | | |
| Breast ca. BT | 6.5 | 7.1 | Fetal Skeletal | 1.0 | 1.1 |
| 549 | | | Muscle | | |
| Breast ca. | 53.6 | 54.0 | Skeletal Muscle | 2.5 | 2.6 |
| T47D | | | Pool | | |
| Breast ca. | 5.8 | 5.6 | Spleen Pool | 16.2 | 15.2 |
| MDA-N | | | | | |
| Breast Pool | 4.6 | 4.9 | Thymus Pool | 5.4 | 5.7 |
| Trachea | 9.8 | 8.9 | CNS cancer | 11.3 | 13.2 |
| | | | (glio/astro) U87- | | |
| | | | MG | | |
| Lung | 2.0 | 2.1 | CNS cancer | 3.1 | 2.8 |
| | | | (glio/astro) U- | | |
| | | | 118-MG | | |
| Fetal Lung | 7.0 | 6.6 | CNS cancer | 2.1 | 2.7 |
| | | | (neuro;net)SK- | | |
| | | | N-AS | | |
| Lung ca. NCI- | 0.7 | 0.6 | CNS cancer | 27.4 | 24.1 |
| N417 | | | (astro) SF-539 | | |
| Lung ca. LX-1 | 3.0 | 4.2 | CNS cancer | 100.0 | 100.0 |
| | | | (astro) SNB-75 | | |
| Lung ca. NCI- | 1.2 | 1.3 | CNS cancer (glio) | 46.3 | 41.8 |
| H146 | | | SNB-19 | | |
| Lung ca. SHP- | 1.7 | 1.4 | CNS cancer (glio) | 62.4 | 66.4 |
| 77 | | | SF-295 | | |
| Lung ca. A549 | 1.6 | 1.1 | Brain (Amygdala) | 1.6 | 1.6 |
| | | | Pool | | |
| Lung ca. NCI- | 2.7 | 3.0 | Brain | 1.3 | 1.5 |
| H526 | | | (cerebellum) | | |
| Lung ca.NCI- | 0.2 | 0.3 | Brain (fetal) | 0.7 | 0.8 |
| H23 | | | | | |
| Lung ca. NCI- | 14.7 | 12.0 | Brain | 1.5 | 1.6 |
| H460 | | | (Hippocampus) | | |
| | | | Pool | | |
| Lung ca. | 19.9 | 21.3 | Cerebral Cortex | 1.2 | 1.2 |
| HOP-62 | | | Pool | | |
| Lung ca. NCI- | 3.8 | 4.3 | Brain (Substantia | 2.5 | 2.6 |
| H522 | | | nigra) Pool | | |
| Liver | 2.6 | 2.8 | Brain (Thalamus) | 1.4 | 1.5 |
| | | | Pool | | |
| Fetal Liver | 1.6 | 1.4 | Brain (whole) | 0.8 | 0.7 |
| Liver ca. | 4.9 | 6.4 | Spinal Cord Pool | 5.3 | 5.0 |
| HepG2 | | | | | |
| Kidney Pool | 12.0 | 11.9 | Adrenal Gland | 4.2 | 5.1 |
| Fetal Kidney | 0.9 | 1.1 | Pituitary gland | 2.8 | 2.5 |
| | | | Pool | | |
| Renal ca. 786-0 | 9.2 | 8.9 | Salivary Gland | 1.7 | 1.6 |
| Renal ca. | 5.2 | 5 0 | Thyroid (female) | 11.8 | 12.2 |
| A498 | | | | | |
| Renal ca. | 3.3 | 3.4 | Pancreatic ca. | 10.4 | 9.7 |
| ACHN | | | CAPAN2 | | |
| Renal ca. UO-31 | 6.7 | 7.8 | Pancreas Pool | 7.0 | 7.5 |

TABLE KE
Panel 4.1 D

| | Rel. | Rel. | | Rel. | Rel. |
|---|---|---|---|---|---|
| | Exp.(%) | Exp.(%) | | Exp.(%) | Exp.(%) |
| | Ag3516, | Ag3517, | | Ag3516, | Ag3517, |
| | Run | Run | | Run | Run |
| Tissue Name | 169840392 | 169840401 | Tissue Name | 169840392 | 169840401 |
| Secondary Th1 act | 24.5 | 24.0 | HUVEC IL-1 beta | 14.0 | 12.6 |
| Secondary Th2 act | 49.3 | 54.7 | HUVEC IFN | 2.9 | 2.8 |
| | | | gamma | | |
| Secondary Tr1 act | 50.7 | 51.1 | HUVEC TNF | 27.2 | 27.4 |
| | | | alpha + IFN | | |
| | | | gamma | | |
| Secondary Th1 rest | 10.5 | 10.1 | HUVEC TNF | 12.3 | 12.1 |
| | | | alpha + IL4 | | |
| Secondary Th2 rest | 13.6 | 12.5 | HUVEC IL-11 | 1.6 | 0.9 |
| Secondary Tr1 rest | 12.1 | 12.0 | Lung | 7.5 | 5.7 |
| | | | Microvascular EC | | |
| | | | none | | |
| Primary Th1 act | 11.2 | 12.2 | Lung | 11.3 | 8.0 |
| | | | Microvascular EC | | |
| | | | TNF alpha + IL- | | |
| | | | 1 beta | | |
| Primary Th2 act | 13.3 | 15.1 | Microvascular | 7.4 | 7.8 |
| | | | Dermal EC none | | |
| Primary Tr1 act | 14.1 | 14.3 | Microvascular | 19.3 | 22.5 |
| | | | Dermal EC | | |
| | | | TNF alpha + IL- | | |
| | | | 1 beta | | |
| Primary Th1 rest | 33.4 | 36.6 | Bronchial | 3.8 | 3.5 |
| | | | epithelium | | |
| | | | TNF alpha + | | |
| | | | IL1 beta | | |
| Primary Th2 rest | 26.8 | 26.2 | Small airway | 3.1 | 2.7 |
| | | | epithelium none | | |
| Primary Tr1 rest | 45.7 | 37.9 | Small airway | 5.8 | 5.2 |
| | | | epithelium | | |
| | | | TNF alpha + IL- | | |
| | | | 1 beta | | |
| CD45RA CD4 | 27.0 | 24.8 | Coronery artery | 7.0 | 6.1 |
| lymphocyte act | | | SMC rest | | |
| CD45RO CD4 | 38.7 | 37.9 | Coronery artery | 4.9 | 5.0 |
| lymphocyte act | | | SMC TNF alpha + | | |
| | | | IL_1 beta | | |
| CD8 lymphocyte | 47.3 | 42.3 | Astrocytes rest | 7.6 | 6.4 |
| act | | | | | |
| Secondary CD8 | 36.9 | 27.9 | Astrocytes | 9.2 | 7.6 |
| lymphocyte rest | | | TNF alpha + IL- | | |
| | | | 1 beta | | |
| Secondary CD8 | 25.7 | 25.0 | KU-812 (Basophil) | 0.9 | 0.6 |
| lymphocyte act | | | rest | | |
| CD4 lymphocyte | 14.0 | 13.1 | KU-812 (Basophil) | 2.4 | 2.2 |
| none | | | PMA/ionomycin | | |
| 2ry | 38.7 | 40.9 | CCD1106 | 2.4 | 2.3 |
| Th1/Th2/Tr1_anti- | | | (Keratinocytes) | | |
| CD95 CH11 | | | none | | |
| LAK cells rest | 43.8 | 37.1 | CCD1106 | 6.3 | 4.1 |
| | | | (Kerationcytes) | | |
| | | | TNF alpha + IL- | | |
| | | | 1 beta | | |
| LAK cells IL-2 | 27.9 | 28.5 | Liver cirrhosis | 6.5 | 6.6 |
| LAK cells IL-2 + | 51.4 | 48.0 | NCI-H292 none | 3.1 | 3.4 |
| IL-12 | | | | | |
| LAK cells IL- | 50.3 | 53.6 | NCI-H292 IL-4 | 3.8 | 3.4 |
| 2 + IFN gamma | | | | | |
| LAK cells IL-2 + | 53.6 | 46.7 | NCI-H292 IL-9 | 4.2 | 2.8 |
| IL-18 | | | | | |
| LAK cells | 48.3 | 41.5 | NCI-H292 IL-13 | 2.5 | 2.4 |
| PMA/ionomycin | | | | | |
| NK Cells IL-2 rest | 63.3 | 58.2 | NCI-H292 IFN | 10.0 | 9.4 |
| | | | gamma | | |
| Two Way MLR 3 | 65.5 | 61.6 | HPAEC none | 4.9 | 3.1 |
| day | | | | | |
| Two Way MLR 5 | 49.0 | 53.6 | HPAEC TNF | 24.7 | 21.0 |
| day | | | alpha + IL-1 beta | | |
| Two Way MLR 7 | 35.4 | 33.2 | Lung fibroblast | 8.8 | 7.1 |
| day | | | none | | |
| PBMC rest | 26.8 | 22.5 | Lung fibroblast | 42.3 | 33.9 |
| | | | TNF alpha + IL-1 | | |
| | | | beta | | |
| PBMC PWM | 42.0 | 29.1 | Lung fibroblast IL-4 | 8.7 | 7.2 |
| PBMC PHA-L | 29.3 | 36.9 | Lung fibroblast IL-9 | 9.9 | 10.7 |
| Ramos (B cell) | 6.9 | 8.0 | Lung fibroblast IL- | 8.8 | 8.2 |
| none | | | 13 | | |
| Ramos (B cell) | 7.1 | 7.7 | Lung fibroblast | 23.2 | 18.8 |
| ionomycin | | | IFN gamma | | |
| B lymphocytes | 27.4 | 20.9 | Dermal fibroblast | 10.7 | 11.3 |
| PWM | | | CCD1070 rest | | |
| B lymphocytes | 54.7 | 40.9 | Dermal fibroblast | 28.5 | 22.4 |
| CD40L and IL-4 | | | CCD1070 TNF | | |
| | | | alpha | | |
| EOL-1 dbcAMP | 2.6 | 3.0 | Dermal fibroblast | 6.7 | 6.3 |
| | | | CCD1070 IL-1 | | |
| | | | beta | | |
| EOL-1 dbcAMP | 2.7 | 2.8 | Dermal fibroblast | 25.0 | 23.2 |
| PMA/ionomycin | | | IFN gamma | | |
| Dendritic cells none | 29.7 | 33.9 | Dermal fibroblast | 13.4 | 13.8 |
| | | | IL-4 | | |
| Dendritic cell LPS | 57.4 | 53.2 | Dermal Fibroblasts | 5.0 | 3.5 |
| | | | rest | | |
| Dendritic cells anti- | 29.7 | 29.9 | Neutrophils | 51.8 | 45.4 |
| CD40 | | | TNFa + LPS | | |
| Monocytes rest | 23.7 | 23.8 | Neutrophils rest | 76.8 | 75.3 |
| Monocytes LPS | 100.0 | 100.0 | Colon | 26.8 | 22.8 |
| Macrophages rest | 25.5 | 23.5 | Lung | 23.2 | 27.0 |
| Macrophages LPS | 61.1 | 60.3 | Thymus | 9.3 | 11.6 |
| HUVEC none | 4.4 | 6.3 | Kidney | 3.7 | 4.2 |
| HUVEC starved | 8.0 | 9.5 | | | |

CNS_neurodegeneration_v1.0 Summary: Ag3516/Ag3517 This panel does not show differential expression of the CG59239-01 gene in Alzheimer's disease. However, this expression profile confirms the presence of this gene in the brain. Please see Panel 1.4 for discussion of utility of this gene in the central nervous system.

General_screening_panel_v1.4 Summary: Ag3516/Ag3517 Two experiments with the same probe and primer set produce results that are in excellent agreement, with highest expression of the CG59239-01 gene in a brain cancer cell line (CT=24). This gene encodes a major histocompatibility complex (MIJC) class I homologue. MHC Class I genes mediate the recognition of intracellular antigens by cytotoxic T cells and are typically ubiquitously expressed. Significant expression is also seen in clusters of cell lines derived from gastric, colon, lung, breast, ovarian and brain cancers. Thus, expression of this gene could be used to differentiate between these samples and other samples on this panel and as a marker to detect the presence of these cancers. Furthermore, therapeutic modulation of the expression or function of this gene may be effective in the treatment of gastric, colon, lung, breast, ovarian and brain cancer.

Among tissues with metabolic function, this gene is expressed at moderate levels in adipose, adrenal gland, pancreas, pituitary, thyroid, and adult and fetal skeletal muscle, heart, and liver. This widespread expression among these tissues suggests that this gene product may play a role in normal metabolic and neuroendocrine function and that disregulated expression of this gene may contribute to neuroendocrine disorders or metabolic diseases, such as obesity and diabetes.

In addition, this gene shows moderate expression in all regions of the CNS examined. Inflammation has been implicated in many CNS neurodegenerative disorders, including Alzheimer's disease. Therefore, therapeutic modulation of the expression or function of this gene may be useful in the treatment of neurodegenerative disorders such as Alzheillmer's disease, Parkinson's disease, and multiple sclerosis.

References:

1. Bauer J, Rauschka H, Lassmann H. Inflammation in the nervous system: the human perspective. Glia November 2001;36(2):235-43

Panel 4.1D Summary: Ag3516/Ag3517 Two experiments with the same probe and primer set produce results that are in excellent agreement, with highest expression of the CG59239-01 gene in LPS-stimulated inonocytes (CT=24). This gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

This gene encodes a major histocompatibility complex (MIHC) class I homolog. MHC class I-like molecules may have utility in forensics, such as in genotyping criminal suspects, and in settling paternity disputes. These antigens are ubiquitously expressed on all nucleated human cells except neurons and trophoblasts, and participate in antigen presentation of viral antigens in the adaptive immune response.

### L. NOV17a (CG59295-0 1: OTOGELIN)

Expression of gene CG59295-01 was assessed using the primer-probe set Ag3534, described in Table LA. Results of the RTQ-PCR runs are shown in Tables LB, and LC.

TABLE LA
Probe Name Ag3534

| | | | | Start |
|---|---|---|---|---|
| Primer | Sequences | | Length | Position |
| Forward | 5′-agctgagtgtccctgtgagtt-3′ | (SEQ ID NO:251) | 21 | 1422 |
| Probe | TET-5′-actctgtacccacctggctctgtggt-3′-TAMRA | (SEQ ID NO:252) | 26 | 1450 |
| Reverse | 5′-tgcaagtattgcagtcttcctt-3′ | (SEQ ID NO:253) | 22 | 1477 |

TABLE LB
General_screening_panel_v1.4

| | Rel. Exp.(%) Ag3534, | | Rel. Exp.(%) Ag3534, |
|---|---|---|---|
| Tissue Name | Run 217044594 | Tissue Name | Run 217044594 |
| Adipose | 0.0 | Renal ca. TK-10 | 0.0 |
| Melanoma* | 0.0 | Bladder | 10.9 |
| Hs688(A).T | | | |
| Melanoma* | 0.0 | Gastric ca. (liver met.) | 11.4 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 20.3 |
| MEL-5 | | | |
| Squamous cell | 0.0 | Colon ca.* (SW480 | 3.7 |
| carcinoma SCC-4 | | met) SW620 | |
| Testis Pool | 25.7 | Colon ca. HT29 | 0.0 |
| Prostate ca.* (bone | 0.0 | Colon ca. HCT-116 | 2.6 |
| met) PC-3 | | | |
| Prostate Pool | 0.0 | Colon ca. CaCo-2 | 100.0 |
| Placenta | 0.0 | Colon cancer tissue | 0.0 |
| Uterus Pool | 0.0 | Colon ca. SW1116 | 0.0 |
| Ovarian ca. OVCAR-3 | 9.2 | Colon ca. Colo-205 | 0.0 |
| Ovarian ca. SK-OV-3 | 0.0 | Colon ca. SW-48 | 2.7 |
| Ovarian ca. OVCAR-4 | 2.5 | Colon Pool | 6.0 |
| Ovarian ca. OVCAR-5 | 11.7 | Small Intestine Pool | 3.0 |
| Ovarian ca. IGROV-1 | 0.0 | Stomach Pool | 3.1 |
| Ovarian ca. OVCAR-8 | 0.0 | Bone Marrow Pool | 0.0 |
| Ovary | 0.0 | Fetal Heart | 2.9 |
| Breast ca. MCF-7 | 0.0 | Heart Pool | 2.8 |
| Breast ca. MDA- | 0.0 | Lymph Node Pool | 2.6 |
| MB-231 | | | |
| Breast ca. BT 549 | 0.0 | Fetal Skeletal Muscle | 0.0 |
| Breast ca. T47D | 23.5 | Skeletal Muscle Pool | 0.0 |
| Breast ca. MDA-N | 0.0 | Spleen Pool | 0.0 |
| Breast Pool | 2.8 | Thymus Pool | 2.6 |
| Trachea | 0.0 | CNS cancer (glio/astro) | 0.0 |
| | | U87-MG | |
| Lung | 0.0 | CNS cancer (glio/astro) | 0.0 |
| | | U-118-MG | |
| Fetal Lung | 0.0 | CNS cancer (neuro;met) | 0.0 |
| | | SK-N-AS | |
| Lung ca. NCI-N417 | 0.0 | CNS cancer (astro) SF- | 0.0 |
| | | 539 | |
| Lung ca. LX-1 | 66.9 | CNS cancer (astro) | 0.0 |
| | | SNB-75 | |
| Lung ca. NCI-H146 | 19.1 | CNS cancer (glio) SNB- | 0.0 |
| | | 19 | |
| Lung ca. SHP-77 | 0.0 | CNS cancer (glio) SF- | 2.8 |
| | | 295 | |
| Lung ca. A549 | 0.0 | Brain (Amygdala) Pool | 2.7 |
| Lung ca. NCI-H526 | 4.8 | Brain (cerebellum) | 0.0 |
| Lung ca. NCI-H23 | 3.9 | Brain (fetal) | 6.3 |
| Lung ca. NCI-H460 | 2.7 | Brain (Hippocampus) | 5.2 |
| | | Pool | |
| Lung ca. HOP-62 | 0.0 | Cerebral Cortex Pool | 0.0 |
| Lung ca. NCI-H522 | 0.0 | Brain (Substantia nigra) | 2.7 |
| | | Pool | |
| Liver | 0.0 | Brain (Thalamus) Pool | 0.0 |
| Fetal Liver | 0.0 | Brain (whole) | 0.0 |
| Liver ca. HepG2 | 1.5 | Spinal Cord Pool | 5.5 |
| Kidney Pool | 0.0 | Adrenal Gland | 0.0 |
| Fetal Kidney | 10.7 | Pituitary gland Pool | 16.7 |
| Renal ca. 786-0 | 0.0 | Salivary Gland | 0.0 |
| Renal ca. A498 | 6.9 | Thyroid (female) | 0.0 |
| Renal ca. ACHN | 0.0 | Pancreatic ca. CAPAN2 | 0.0 |
| Renal ca. UO-31 | 0.0 | Pancreas Pool | 4.3 |

TABLE LC
Panel 4D

| | Rel. Exp.(%) Ag3534, | | Rel. Exp.(%) Ag3534, |
|---|---|---|---|
| Tissue Name | Run 166444750 | Tissue Name | Run 166444750 |
| Secondary Th1 act | 2.4 | HUVEC IL-1 beta | 0.0 |
| Secondary Th2 act | 2.0 | HUVEC IFN gamma | 0.0 |
| Secondary Tr1 act | 2.5 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1 beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1 beta | |
| Primary Th1 rest | 0.0 | Bronchial epithelium | 0.0 |
| | | TNF alpha + IL1 beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1 beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1 beta | |
| CD8 lymphocyte act | 0.0 | Astrocytes rest | 0.0 |
| Secondary CD8 | 0.0 | Astrocytes TNF alpha + IL- | 0.0 |
| lymphocyte rest | | 1 beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1 beta | |
| LAK cells IL-2 | 0.0 | Liver cirrhosis | 100.0 |
| LAK cells IL-2 + IL-12 | 0.0 | Lupus kidney | 6.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 1.5 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF | 0.0 |
| | | alpha + IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1 beta | |
| Dendritic cells none | 2.7 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 4.4 |
| CD40 | | | |
| Monocytes rest | 0.0 | IBD Crohn's | 0.0 |
| Monocytes LPS | 0.0 | Colon | 44.4 |
| Macrophages rest | 0.0 | Lung | 15.9 |
| Macrophages LPS | 0.0 | Thymus | 10.0 |
| HUVEC none | 0.0 | Kidney | 0.0 |
| HUVEC starved | 0.0 | | |

CNS_neurodegeneration_v1.0 Summary: Ag3534 Expression of the CG59295-01 gene is low/undetectable in all samples on this panel (CTs>35).

General_screening_panel_v1.4 Summary: Ag3534 Highest expression of the CG59295-01 gene is seen in a colon cancer cell line (CT=3 1). Low but significant levels of expression are seen in lung and breast cancer cell lines. Therefore, expression of this gene could be used to differentiate between these samples and other samples on this panel and as a marker for colon cancer. This gene has been linked to deafness. Thus, therapeutic modulation of the expression or function of this gene may be effective in the treatment of deafness.

References:

Simmler M C, Cohen-Salmon M, El-Amraoui A, Guillaud L, Benichou J C, Petit C, Panthier J J.

Panel 4D Summary: Ag3534 Expression of the CG59295-01 gene is restricted to a few samples, with highest expression in liver cirrhosis (CT=32.2). Low but significant level of expression are also seen in lung and colon. In addition, expression is undetectable in colon from patients with IBD colitis and Crohn's and in liver on Panel 1.4. Thus, expression of this gene may be used to differentiate between normal and diseased liver and colon tissue. Also, therapeutic modulation of the activity of the otogelin protein encoded by this gene may be useful in the treatment of inflammatory bowel disease and inflammatory or autoimmune diseases which affect the liver including liver cirrhosis and fibrosis.

Panel CNS_{—}1 Summary: Ag3534 Expression of the CG59295-01 gene is low/undetectable in all samples on this panel (CTs>35).

### M. NOV18a (CG59293-01: Renal Organic Anion Transport Protein 1)

Expression of gene CG59293-01 was assessed using the primer-probe sets Ag3948, Ag3532 and Ag2874, described in Tables MA, MB and MC. Results of the RTQ-PCR runs are shown in Tables MD, ME, MF, MG, MH, MI, MJ and MK.

TABLE MA
Probe Name Ag3948

| | | | | Start |
|---|---|---|---|---|
| Primer | Sequences | | Length | Position |
| Forward | 5′-ctctattcttggtggcttcca-3′ | (SEQ ID NO:254) | 21 | 867 |
| Probe | TET-5′-ctcctgcatggcaagtcccagttag-3′-TAMRA | (SEQ ID NO:255) | 25 | 906 |
| Reverse | 5′-ccaccttctgcagattctgtac-3′ | (SEQ ID NO:256) | 22 | 993 |

TABLE MB
Probe Name Ag3532

| | | | | Start |
|---|---|---|---|---|
| Primer | Sequences | | Length | Position |
| Forward | 5′-ccaactcaatcttggacctctt-3′ | (SEQ ID NO:257) | 22 | 1048 |
| Probe | TET-5′-atccgcaaggtcacatgctgtctcat-3′-TAMRA | (SEQ ID NO:258) | 26 | 1083 |
| Reverse | 5′-cagagttggagaaccaaatcac-3′ | (SEQ ID NO:259) | 22 | 1110 |

TABLE MC

| | | | |
|---|---|---|---|
| Probe Name Ag2874 | | | |
| | | | Start |
| Primers | Sequences | Length | Position |

| | | | | | | |
|---|---|---|---|---|---|---|
| Forward | 5′-ccaactcaatcttggacctctt-3′ | (SEQ ID NO:260) | 22 | 1048 | | |
| Probe | TET-5′-atccgcaaggtcacatgctgtctcat-3′-TAMRA | (SEQ ID NO:261) | 26 | 1083 | JNO:261) | I |
| Reverse | 5′-cagagttggagaaccaaatcac-3′ | (SEQ ID NO:262) | 22 | 1110 | | |

TABLE MD
AI_comprehensive panel_v1.0

| | Rel. Exp.(%) Ag3948, | | Rel. Exp.(%) Ag3948, |
|---|---|---|---|
| Tissue Name | Run 247842315 | Tissue Name | Run 247842315 |
| 110967 COPD-F | 17.8 | 112427 Match Control | 12.1 |
| | | Psoviasis-F | |
| 110980 COPD-F | 6.7 | 112418 Psoriasis-M | 11.2 |
| 110968 COPD-M | 9.2 | 112723 Match Control | 9.6 |
| | | Psoriasis-M | |
| 110977 COPD-M | 20.0 | 112419 Psoriasis-M | 30.6 |
| 110989 Emphysema-F | 34.6 | 112424 Match Control | 18.4 |
| | | Psoriasis-M | |
| 110992 Emphysema-F | 0.0 | 112420 Psoriasis-M | 94.6 |
| 110993 Emphysema-F | 18.6 | 112425 Match Control | 42.6 |
| | | Psoriasis-M | |
| 110994 Emphysema-F | 9.2 | 104689 (MF) OA Bone- | 20.2 |
| | | Backus | |
| 110995 Emphysema-F | 100.0 | 104690 (MF) Adj | 7.2 |
| | | “Normal” Bone-Backus | |
| 110996 Emphysema-F | 5.2 | 104691 (MF) OA | 10.0 |
| | | Synovium-Backus | |
| 110997 Asthma-M | 18.0 | 104692 (BA) OA | 0.0 |
| | | Cartilage-Backus | |
| 111001 Asthma-F | 0.0 | 104694 (BA) OA Bone- | 14.2 |
| | | Backus. | |
| 111002 Asthma-F | 8.8 | 104695 (BA) Adj | 7.5 |
| | | “Normal” Bone-Backus | |
| 111003 Atopic | 5.6 | 104696 (BA) OA | 13.9 |
| Asthma-F | | Synovium-Backus | |
| 111004 Atopic | 41.5 | 104700 (SS) OA Bone- | 35.6 |
| Asthma-F | | Backus | |
| 111005 Atopic | 17.6 | 104701 (SS) Adj | 8.7 |
| Asthma-F | | “Normal” Bone-Backus | |
| 111006 Atopic | 0.0 | 104702 (SS) OA | 26.8 |
| Asthma-F | | Synovium-Backus | |
| 111417 Allergy-M | 2.4 | 117093 OA Cartilage | 18.9 |
| | | Rep7 | |
| 112347 Allergy-M | 0.0 | 112672 OA Bone5 | 13.4 |
| 112349 Normal Lung-F | 10.1 | 112673 OA Synovium5 | 0.0 |
| 112357 Normal Lung-F | 9.2 | 112674 OA Synovial | 5.8 |
| | | Fluid cells5 | |
| 112354 Normal Lung-M | 19.2 | 117100 OA Cartilage | 14.8 |
| | | Rep14 | |
| 112374 Crohns-F | 28.3 | 112756 OA Bone9 | 21.0 |
| 112389 Match Control | 15.2 | 112757 OA Synovium9 | 20.3 |
| Crohns-F | | | |
| 112375 Crohns-F | 14.3 | 112758 OA Synovial | 7.2 |
| | | Fluid Cells9 | |
| 112732 Match Control | 92.7 | 117125 RA Cartilage | 25.9 |
| Crohns-F | | Rep2 | |
| 112725 Crohns-M | 23.5 | 113492 Bone2 RA | 10.9 |
| 112387 Match Control | 13.6 | 113493 Synovium2 RA | 8.8 |
| Crohns-M | | | |
| 112378 Crohns-M | 14.4 | 113494 Syn Fluid Cells | 16.8 |
| | | RA | |
| 112390 Match Control | 72.7 | 113499 Cartilage4 RA | 12.9 |
| Crohns-M | | | |
| 112726 Crohns-M | 12.8 | 113500 Bone4 RA | 20.7 |
| 112731 Match Control | 65.5 | 113501 Synovium4 RA | 13.8 |
| Crohns-M | | | |
| 112380 Ulcer Col-F | 18.6 | 113502 Syn Fluid | 3.1 |
| | | Cells4 RA | |
| 112734 Match Control | 89.5 | 113495 Cartilage3 RA | 6.8 |
| Ulcer Col-F | | | |
| 112384 Ulcer Col-F | 57.8 | 113496 Bone3 RA | 20.7 |
| 112737 Match Control | 15.6 | 113497 Synovium3 RA | 8.7 |
| Ulcer Col-F | | | |
| 112386 Ulcer Col-F | 2.7 | 113498 Syn Fluid | 21.6 |
| | | Cells3 RA | |
| 112738 Match Control | 4.3 | 117106 Normal | 5.7 |
| Ulcer Col-F | | Cartilage Rep20 | |
| 112381 Ulcer Col-M | 0.0 | 113663 Bone3 Normal | 16.2 |
| 112735 Match Control | 58.2 | 113664 Synovium3 | 2.6 |
| Ulcer Col-M | | Normal | |
| 112382 Ulcer Col-M | 8.4 | 113665 Syn Fluid | 5.2 |
| | | Cells3 Normal | |
| 112394 Match Control | 17.1 | 117107 Normal | 11.0 |
| Ulcer Col-M | | Cartilage Rep22 | |
| 112383 Ulcer Col-M | 47.3 | 113667 Bone4 Normal | 15.4 |
| 112736 Match Control | 1.9 | 113668 Synovium4 | 20.2 |
| Ulcer Col-M | | Normal | |
| 112423 Psoriasis-F | 27.4 | 113669 Syn Fluid | 40.9 |
| | | Cells4 Normal | |

TABLE ME
CNS_neurodegeneration_v1.0

| | Rel. Exp.(%) Ag3948, | | Rel. Exp.(%) Ag3948, |
|---|---|---|---|
| Tissue Name | Run 212345604 | Tissue Name | Run 212345604 |
| AD 1 Hippo | 0.0 | Control (Path) 3 | 0.0 |
| | | Temporal Ctx | |
| AD 2 Hippo | 57.0 | Control (Path) 4 | 33.4 |
| | | Temporal Ctx | |
| AD 3 Hippo | 32.5 | AD 1 Occipital Ctx | 67.8 |
| AD 4 Hippo | 0.0 | AD 2 Occipital Ctx | 5.3 |
| | | (Missing) | |
| AD 5 Hippo | 47.0 | AD 3 Occipital Ctx | 0.0 |
| AD 6 Hippo | 20.3 | AD 4 Occipital Ctx | 0.0 |
| Control 2 Hippo | 0.0 | AD 5 Occipital Ctx | 18.4 |
| Control 4 Hippo | 0.0 | AD 6 Occipital Ctx | 39.2 |
| Control (Path) 3 | 0.0 | Control 1 Occipital | 0.0 |
| Hippo | | Ctx | |
| AD 1 Temporal Ctx | 0.0 | Control 2 Occipital | 79.6 |
| | | Ctx | |
| AD 2 Temporal Ctx | 16.7 | Control 3 Occipital | 42.0 |
| | | Ctx | |
| AD 3 Temporal Ctx | 14.9 | Control 4 Occipital | 0.0 |
| | | Ctx | |
| AD 4 Temporal Ctx | 0.0 | Control (Path) 1 | 46.0 |
| | | Occipital Ctx | |
| AD 5 Inf Temporal | 100.0 | Control (Path) 2 | 0.0 |
| Ctx | | Occipital Ctx | |
| AD 5 Sup Temporal | 0.0 | Control (Path) 3 | 0.0 |
| Ctx | | Occipital Ctx | |
| AD 6 Inf Temporal | 65.1 | Control (Path) 4 | 11.7 |
| Ctx | | Occipital Ctx | |
| AD 6 Sup Temporal | 91.4 | Control 1 Parietal | 10.3 |
| Ctx | | Ctx | |
| Control 1 Temporal | 0.0 | Control 2 Parietal | 49.3 |
| Ctx | | Ctx | |
| Control 2 Temporal | 0.0 | Control 3 Parietal | 0.0 |
| Ctx | | Ctx | |
| Control 3 Temporal | 0.0 | Control (Path) 1 | 67.8 |
| Ctx | | Parietal Ctx | |
| Control 3 Temporal | 18.0 | Control (Path) 2 | 14.4 |
| Ctx | | Parietal Ctx | |
| Control (Path) 1 | 32.1 | Control (Path) 3 | 0.0 |
| Temporal Ctx | | Parietal Ctx | |
| Control (Path) 2 | 9.7 | Control (Path) 4 | 20.4 |
| Temporal Ctx | | Parietal Ctx | |

TABLE MF
General_screening_panel_v1.4

| | Rel. Exp.(%) Ag3948, | | Rel. Exp.(%) Ag3948, |
|---|---|---|---|
| Tissue Name | Run 219279808 | Tissue Name | Run 219279808 |
| Adipose | 0.3 | Renal ca. TK-10 | 2.1 |
| Melanoma* | 0.5 | Bladder | 2.8 |
| Hs688(A).T | | | |
| Melanoma* | 0.2 | Gastric ca. (liver met.) | 22.2 |
| Hs688(B).T | | NCI-N87 | |
| Melanoma* M14 | 0.2 | Gastric ca. KATO III | 4.0 |
| Melanoma* | 0.0 | Colon ca. SW-948 | 0.1 |
| LOXIMVI | | | |
| Melanoma* SK- | 0.1 | Colon ca. SW480 | 10.5 |
| MEL-5 | | | |
| Squamous cell | 2.4 | Colon ca.* (SW480 | 1.6 |
| carcinoma SCC-4 | | met) SW620 | |
| Testis Pool | 0.3 | Colon ca. HT29 | 1.5 |
| Prostate ca.* (bone | 0.9 | Colon ca. HCT-116 | 2.3 |
| met) PC-3 | | | |
| Prostate Pool | 0.3 | Colon ca. CaCo-2 | 0.7 |
| Placenta | 0.2 | Colon cancer tissue | 0.2 |
| Uterus Pool | 0.1 | Colon ca. SW1116 | 0.1 |
| Ovarian ca. OVCAR-3 | 11.5 | Colon ca. Colo-205 | 0.6 |
| Ovarian ca. SK-OV-3 | 1.3 | Colon ca. SW-48 | 0.8 |
| Ovarian ca. OVCAR-4 | 0.4 | Colon Pool | 0.6 |
| Ovarian ca. OVCAR-5 | 8.0 | Small Intestine Pool | 0.4 |
| Ovarian ca. IGROV-1 | 1.9 | Stomach Pool | 0.1 |
| Ovarian ca. OVCAR-8 | 0.5 | Bone Marrow Pool | 0.4 |
| Ovary | 0.4 | Fetal Heart | 0.1 |
| Breast ca. MCF-7 | 3.2 | Heart Pool | 0.3 |
| Breast ca. MDA- | 2.3 | Lymph Node Pool | 0.7 |
| MB-231 | | | |
| Breast ca. BT 549 | 0.2 | Fetal Skeletal Muscle | 0.6 |
| Breast ca. T47D | 9.6 | Skeletal Muscle Pool | 0.4 |
| Breast ca. MDA-N | 0.2 | Spleen Pool | 0.2 |
| Breast Pool | 0.7 | Thymus Pool | 0.3 |
| Trachea | 0.6 | CNS cancer (glio/astro) | 0.5 |
| | | U87-MG | |
| Lung | 0.2 | CNS cancer (glio/astro) | 0.1 |
| | | U-118-MG | |
| Fetal Lung | 1.7 | CNS cancer (neuro;met) | 0.4 |
| | | SK-N-AS | |
| Lung ca. NCI-N417 | 0.1 | CNS cancer (astro) SF- | 0.1 |
| | | 539 | |
| Lung ca. LX-1 | 100.0 | CNS cancer (astro) | 0.1 |
| | | SNB-75 | |
| Lung ca. NCI-H146 | 1.0 | CNS cancer (glio) SNB- | 0.9 |
| | | 19 | |
| Lung ca. SHP-77 | 0.1 | CNS cancer (glio) SF- | 9.3 |
| | | 295 | |
| Lung ca. A549 | 2.2 | Brain (Amygdala) Pool | 0.1 |
| Lung ca. NCI-H526 | 0.1 | Brain (cerebellum) | 5.1 |
| Lung ca. NCI-H23 | 0.9 | Brain (fetal) | 2.3 |
| Lung ca. NCI-H460 | 0.2 | Brain (Hippocampus) | 0.0 |
| | | Pool | |
| Lung ca. HOP-62 | 0.5 | Cerebral Cortex Pool | 0.0 |
| Lung ca. NCI-H522 | 1.1 | Brain (Substantia nigra) | 0.2 |
| | | Pool | |
| Liver | 0.3 | Brain (Thalamus) Pool | 0.0 |
| Fetal Liver | 0.8 | Brain (whole) | 0.8 |
| Liver ca. HepG2 | 0.2 | Spinal Cord Pool | 0.0 |
| Kidney Pool | 0.7 | Adrenal Gland | 0.2 |
| Fetal Kidney | 0.7 | Pituitary gland Pool | 0.0 |
| Renal ca. 786-0 | 0.2 | Salivary Gland | 0.1 |
| Renal ca. A498 | 0.2 | Thyroid (female) | 0.7 |
| Renal ca. ACHN | 0.2 | Pancreatic ca. CAPAN2 | 3.8 |
| Renal ca. UO-31 | 1.6 | Pancreas Pool | 0.6 |

TABLE MG
Panel 1.3D

| | Rel. Exp.(%) | Rel. Exp.(%) | | Rel. Exp.(%) | Rel. Exp.(%) |
|---|---|---|---|---|---|
| | Ag2874, Run | Ag2874, Run | | Ag2874, Run | Ag2874, Run |
| Tissue Name | 161973724 | 165721687 | Tissue Name | 161973724 | 165721687 |
| Liver | 4.9 | 5.6 | Kidney (fetal) | 0.0 | 0.0 |
| adenocarcinoma | | | | | |
| Pancreas | 0.0 | 0.0 | Renal ca. 786-0 | 0.0 | 0.0 |
| Pancreatic ca. | 0.0 | 0.0 | Renal ca. A498 | 0.0 | 0.0 |
| CAPAN 2 | | | | | |
| Adrenal gland | 0.0 | 0.0 | Renal ca RXF | 0.0 | 0.0 |
| | | | 393 | | |
| Thyroid | 0.0 | 6.3 | Renal ca. | 0.0 | 0.0 |
| | | | ACHN | | |
| Salivary gland | 0.0 | 0.0 | Renal ca. UO- | 0.0 | 0.0 |
| | | | 31 | | |
| Pituitary gland | 0.0 | 0.0 | Renal ca. TK- | 0.0 | 0.0 |
| | | | 10 | | |
| Brain (fetal) | 0.0 | 3.8 | Liver | 0.0 | 3.1 |
| Brain (whole) | 3.1 | 6.1 | Liver (fetal) | 0.0 | 0.0 |
| Brain (amygdala) | 4.3 | 0.0 | Liver ca. | 0.0 | 0.0 |
| | | | (hepatoblast) | | |
| | | | HepG2 | | |
| Brain (cerebellum) | 1.8 | 7.6 | Lung | 0.0 | 0.0 |
| Brain | 0.0 | 0.0 | Lung (fetal) | 4.5 | 4.1 |
| (hippocampus) | | | | | |
| Brain (substantia | 0.0 | 3.5 | Lung ca. | 100.0 | 100.0 |
| nigra) | | | (small cell) | | |
| | | | LX-1 | | |
| Brain (thalamus) | 0.0 | 0.0 | Lung ca. | 0.0 | 3.2 |
| | | | (small cell) | | |
| | | | NCI-H69 | | |
| Cerebral Cortex | 2.3 | 0.0 | Lang ca. (s.cell | 0.0 | 0.0 |
| | | | var.) SHP-77 | | |
| Spinal cord | 0.0 | 0.0 | Lung ca. (large | 0.0 | 0.0 |
| | | | cell) NCI-H460 | | |
| glio/astro U87-MG | 0.0 | 0.0 | Lung ca. (non- | 0.0 | 0.0 |
| | | | sm. cell) A549 | | |
| glio/astro U-118- | 0.0 | 0.0 | Lung ca. (non- | 0.0 | 0.0 |
| MG | | | s.cell) NCI- | | |
| | | | H23 | | |
| astrocytoma | 0.0 | 0.0 | Lung ca. (non- | 0.0 | 0.0 |
| SW1783 | | | s.cell) HOP-62 | | |
| neuro*; met SK-N- | 0.0 | 0.0 | Lung ca. (non- | 1.7 | 0.0 |
| AS | | | s.cl) NCI-H522 | | |
| astrocytoma SF- | 3.8 | 0.0 | Lung ca. | 2.1 | 0.0 |
| 539 | | | (squam.) SW | | |
| | | | 900 | | |
| astrocytoma SNB- | 0.0 | 0.0 | Lung ca. | 0.0 | 0.0 |
| 75 | | | (squam.) NCI- | | |
| | | | H596 | | |
| glioma SNB-19 | 0.0 | 0.0 | Mammary | 0.0 | 0.0 |
| | | | gland | | |
| glioma U251 | 0.0 | 0.0 | Breast ca.* | 13.9 | 0.0 |
| | | | (pl.ef) MCF-7 | | |
| glioma SF-295 | 10.7 | 12.5 | Breast ca.* | 2.6 | 0.0 |
| | | | (pl.et) MDA- | | |
| | | | MB-231 | | |
| Heart (fetal) | 0.0 | 0.0 | Breast ca.* | 3.2 | 4.7 |
| | | | (pl.ef) T47D | | |
| Heart | 0.0 | 0.0 | Breast ca. BT-549 | 0.0 | 0.0 |
| Skeletal muscle | 0.0 | 5.3 | Breast ca. | 0.0 | 0.0 |
| (fetal) | | | MDA-N | | |
| Skeletal muscle | 0.0 | 0.0 | Ovary | 0.0 | 0.0 |
| Bone marrow | 17.9 | 6.1 | Ovarian ca. | 24.1 | 4.7 |
| | | | OVCAR-3 | | |
| Thymus | 18.7 | 0.0 | Ovarian ca. | 4.7 | 0.0 |
| | | | OVCAR-4 | | |
| Spleen | 0.0 | 0.0 | Ovarian ca. | 4.8 | 4.8 |
| | | | OVCAR-5 | | |
| Lymph node | 10.5 | 0.0 | Ovarian ca. | 0.0 | 0.0 |
| | | | OVCAR-8 | | |
| Colorectal | 4.9 | 3.1 | Ovarian ca. | 3.0 | 0.0 |
| | | | IGROV-1 | | |
| Stomach | 0.0 | 0.0 | Ovarian ca.* | 0.0 | 3.4 |
| | | | (ascites) SK- | | |
| | | | OV-3 | | |
| Small intestine | 0.0 | 0.0 | Uterus | 0.0 | 6.5 |
| Colon ca. SW480 | 2.7 | 0.0 | Placenta | 0.0 | 0.0 |
| Colon ca.* | 0.0 | 0.0 | Prostate | 10.8 | 2.7 |
| SW620(SW480 | | | | | |
| met) | | | | | |
| Colon ca. HT29 | 0.0 | 0.0 | Prostate ca.* | 0.0 | 0.0 |
| | | | (bone met)PC-3 | | |
| Colon Ca. HCT- | 0.0 | 0.0 | Testis | 0.0 | 0.0 |
| 116 | | | | | |
| Colon ca. CaCo-2 | 0.0 | 0.0 | Melanoma | 0.0 | 0.0 |
| | | | Hs688(A).T | | |
| Colon ca. | 8.8 | 0.0 | Melanoma* | 0.0 | 0.0 |
| tissue(ODO3866) | | | (met) | | |
| | | | Hs688(B).T | | |
| Colon Ca. HCC- | 17.1 | 2.7 | Melanoma | 0.0 | 0.0 |
| 2998 | | | UACC-62 | | |
| Gastric ca.* (liver | 28.9 | 13.4 | Melanoma | 0.0 | 0.0 |
| met) NCI-N87 | | | M14 | | |
| Bladder | 13.6 | 0.0 | Melanoma | 0.0 | 0.0 |
| | | | LOX IMVI | | |
| Trachea | 6.7 | 5.6 | Melanoma * | 0.0 | 0.0 |
| | | | (met) SK- | | |
| | | | MEL-5 | | |
| Kidney | 2.6 | 0.0 | Adipose | 0.0 | 0.0 |

TABLE MH
Panel 2D

| | Rel. Exp.(%) Ag2874, | | Rel. Exp.(%) Ag2874, |
|---|---|---|---|
| Tissue Name | Run 161973958 | Tissue Name | Run 161973958 |
| Normal Colon | 24.3 | Kidney Margin | 0.0 |
| | | 8120608 | |
| CC Well to Mod Diff | 0.0 | Kidney Cancer | 0.0 |
| (ODO3866) | | 8120613 | |
| CC Margin (ODO3866) | 26.2 | Kidney Margin | 21.3 |
| | | 8120614 | |
| CC Gr.2 rectosigmoid | 0.0 | Kidney Cancer | 0.0 |
| (ODO3868) | | 9010320 | |
| CC Margin (ODO3868) | 13.8 | Kidney Margin | 0.0 |
| | | 9010321 | |
| CC Mod Diff (ODO3920) | 11.7 | Normal Uterus | 0.0 |
| CC Margin (ODO3920) | 0.0 | Uterus Cancer 064011 | 10.8 |
| CC Gr.2 ascend colon | 65.1 | Normal Thyroid | 20.2 |
| (ODO3921) | | | |
| CC Margin (ODO3921) | 0.0 | Thyroid Cancer | 42.6 |
| | | 064010 | |
| CC from Partial | 27.9 | Thyroid Cancer | 24.1 |
| Hepatectomy (ODO4309) | | A302152 | |
| Mets | | | |
| Liver Margin (ODO4309) | 0.0 | Thyroid Margin | 0.0 |
| | | A302153 | |
| Colon mets to lung | 7.5 | Normal Breast | 0.0 |
| (OD04451-01) | | | |
| Lung Margin (OD04451- | 0.0 | Breast Cancer | 0.0 |
| 02) | | (OD04566) | |
| Normal Prostate 6546-1 | 13.0 | Breast Cancer | 49.7 |
| | | (OD04590-01) | |
| Prostate Cancer | 0.0 | Breast Cancer Mets | 18.8 |
| (OD04410) | | (OD04590-03) | |
| Prostate Margin | 0.0 | Breast Cancer | 58.2 |
| (OD04410) | | Metastasis (OD04655- | |
| | | 05) | |
| Prostate Cancer | 59.5 | Breast Cancer 064006 | 40.9 |
| (OD04720-01) | | | |
| Prostate Margin | 28.7 | Breast Cancer 1024 | 100.0 |
| (OD04720-02) | | | |
| Normal Lung 061010 | 0.0 | Breast Cancer | 9.7 |
| | | 9100266 | |
| Lung Met to Muscle | 0.0 | Breast Margin | 43.5 |
| (ODO4286) | | 9100265 | |
| Muscle Margin | 0.0 | Breast Cancer | 32.5 |
| (ODO4286) | | A209073 | |
| Lung Malignant Cancer | 0.0 | Breast Margin | 13.4 |
| (OD03126) | | A209073 | |
| Lung Margin (OD03126) | 11.3 | Normal Liver | 0.0 |
| Lung Cancer (OD04404) | 0.0 | Liver Cancer 064003 | 0.0 |
| Lung Margin (OD04404) | 0.0 | Liver Cancer 1025 | 0.0 |
| Lung Cancer (OD04565) | 16.3 | Liver Cancer 1026 | 0.0 |
| Lung Margin (OD04565) | 0.0 | Liver Cancer 6004-T | 0.0 |
| Lung Cancer (OD04237- | 0.0 | Liver Tissue 6004-N | 0.0 |
| 01) | | | |
| Lung Margin (OD04237- | 0.0 | Liver Cancer 6005-T | 0.0 |
| 02) | | | |
| Ocular Mel Met to Liver | 0.0 | Liver Tissue 6005-N | 0.0 |
| (ODO4310) | | | |
| Liver Margin (ODO4310) | 0.0 | Normal Bladder | 58.2 |
| Melanoma Mets to Lung | 0.0 | Bladder Cancer 1023 | 0.0 |
| (OD04321) | | | |
| Lung Margin (OD04321) | 0.0 | Bladder Cancer | 16.6 |
| | | A302173 | |
| Normal Kidney | 16.5 | Bladder Cancer | 66.0 |
| | | (OD04718-01) | |
| Kidney Ca, Nuclear grade | 16.0 | Bladder Normal | 0.0 |
| 2 (OD04338) | | Adjacent (OD04718- | |
| | | 03) | |
| Kidney Margin | 14.1 | Normal Ovary | 0.0 |
| (OD04338) | | | |
| Kidney Ca Nuclear grade | 13.9 | Ovarian Cancer | 85.9 |
| 1/2 (OD04339) | | 064008 | |
| Kidney Margin | 13.0 | Ovarian Cancer | 0.0 |
| (OD04339) | | (OD04768-07) | |
| Kidney Ca, Clear cell type | 0.0 | Ovary Margin | 0.0 |
| (OD04340) | | (OD04768-08) | |
| Kidney Margin | 0.0 | Normal Stomach | 14.7 |
| (OD04340) | | | |
| Kidney Ca, Nuclear grade | 14.8 | Gastric Cancer | 0.0 |
| 3 (OD04348) | | 9060358 | |
| Kidney Margin | 0.0 | Stomach Margin | 0.0 |
| (OD04348) | | 9060359 | |
| Kidney Cancer | 0.0 | Gastric Cancer | 0.0 |
| (OD04622-01) | | 9060395 | |
| Kidney Margin | 0.0 | Stomach Margin | 14.0 |
| (OD04622-03) | | 9060394 | |
| Kidney Cancer | 0.0 | Gastric Cancer | 17.2 |
| (OD04450-01) | | 9060397 | |
| Kidney Margin | 19.2 | Stomach Margin | 0.0 |
| (OD04450-03) | | 9060396 | |
| Kidney Cancer 8120607 | 23.5 | Gastric Cancer 064005 | 20.3 |

TABLE MI
Panel 3D

| | Rel. Exp.(%) | | Rel. Exp.(%) |
|---|---|---|---|
| | Ag2874, Run | | Ag2874, Run |
| Tissue Name | 164543575 | Tissue Name | 164543575 |
| Daoy-Medulloblastoma | 0.0 | Ca Ski-Cervical epidermoid | 0.0 |
| | | carcinoma (metastasis) | |
| TE671-Medulloblastoma | 0.0 | ES-2-Ovarian clear cell- | 0.0 |
| | | carcinoma | |
| D283 Med- | 0.0 | Ramos-Stimulated with | 0.0 |
| Medulloblastoma | | PMA/ionomycin 6 h | |
| PFSK-1-Primitive | 0.0 | Ramos-Stimulated with | 0.0 |
| Neuroectodermal | | PMA/ionomycin 14 h | |
| XF-498-CNS | 0.0 | MEG-01-Chronic | |
| | | myelogenous leukemia | 0.0 |
| | | (megokaryoblast) | |
| SNB-78-Glioma | 0.0 | Raji-Burkitt's lymphoma | 3.3 |
| SF-268-Glioblastoma | 0.0 | Daudi-Burkitt's lymphoma | 0.0 |
| T98G-Glioblastoma | 0.0 | U266-B-cell plasmacytoma | 0.0 |
| SK-N-SH- | 0.0 | CA46-Burkitt's lymphoma | 0.0 |
| Neuroblastoma | | | |
| (metastasis) | | | |
| SF-295-Glioblastoma | 0.0 | RL-non-Hodgkin's B-cell | 0.0 |
| | | lymphoma | |
| Cerebellum | 0.5 | JM1-pre-B-cell lymphoma | 0.0 |
| Cerebellum | 2.3 | Jurkat-T cell leukemia | 0.0 |
| NCI-H292- | 16.8 | TF-1-Erythroleukemia | 0.0 |
| Mucoepidermoid lung | | | |
| carcinoma | | | |
| DMS-114-Small cell | 0.0 | HUT 78-T-cell lymphoma | 8.2 |
| lung cancer | | | |
| DMS-79-Small cell lung | 4.2 | U937-Histiocytic lymphoma | 0.0 |
| cancer | | | |
| NCI-H146-Small cell | 0.0 | KU-812-Myelogenous | 0.0 |
| lung cancer | | leukemia | |
| NCI-H526-Small cell | 0.0 | 769-P-Clear cell renal | 0.0 |
| lung cancer | | carcinoma | |
| NCI-N417-Small cell | 0.0 | Caki-2-Clear cell renal | 0.0 |
| lung cancer | | carcinoma | |
| NCI-H82-Small cell lung | 0.0 | SW 839-Clear cell renal | 2.9 |
| cancer | | carcinoma | |
| NCI-H157-Squamous | 0.0 | G401-Wilm's tumor | 0.0 |
| cell lung cancer | | | |
| (metastasis) | | | |
| NCI-H1155-Large cell | 0.0 | Hs766GT-Pancreatic carcinoma | 3.6 |
| lung cancer | | (LN metastasis) | |
| NCI-H1299-Large cell | 16.6 | CAPAN-1-Pancreatic | 4.4 |
| lung cancer | | adenocarcinoma (liver | |
| | | metastasis) | |
| NCI-H727-Lung | 0.0 | SU86.86-Pancreatic | 100.0 |
| carcinoid | | carcinoma (liver metastasis) | |
| NCI-UMC-11-Lung | 0.0 | BxPC-3-Pancreatic | 0.0 |
| carcinoid | | adenocarcinoma | |
| LX-1-Small cell lung | 48.0 | HPAC-Pancreatic | 3.6 |
| cancer | | adenocarcinoma | |
| Colo-205-Colon cancer | 9.9 | MIA PaCa-2-Pancreatic | 0.0 |
| | | carcinoma | |
| KM12-Colon cancer | 0.0 | CFPAC-1-Pancreatic ductal | 4.4 |
| | | adenocarcinoma | |
| KM20L2-Colon cancer | 0.0 | PANC-1-Pancreatic | 0.0 |
| | | epithelioid ductal carcinoma | |
| NCI-H716-Colon cancer | 0.0 | T24-Bladder carcinoma | 3.9 |
| | | (transitional cell) | |
| SW-48-Colon | 0.0 | 5637-Bladder carcinoma | 0.0 |
| adenocarcinoma | | | |
| SW1116-Colon | 0.0 | HT-1197-Bladder carcinoma | 0.0 |
| adenocarcinoma | | | |
| LS 174T-Colon | 8.3 | UM-UC-3-Bladder carcinma | 0.0 |
| adenocarcinoma | | (transitional cell) | |
| SW-948-Colon | 5.1 | A204-Rhabdomyosarcoma | 0.0 |
| adenocarcinoma | | | |
| SW-480-Colon | 0.0 | HT-1080-Fibrosarcoma | 0.0 |
| adenocarcinoma | | | |
| NCI-SNU-5-Gastric | 13.5 | MG-63-Osteosarcoma | 0.0 |
| carcinoma | | | |
| KATO III-Gastric | 0.0 | SK-LMS-1-Leiomyosarcoma | 4.1 |
| carcinoma | | (vulva) | |
| NCI-SNU-16-Gastric | 0.0 | SJRH30-Rhabdomyosarcoma | 0.0 |
| carcinoma | | (met to bone marrow) | |
| NCI-SNU-1-Gastric | 0.0 | A431-Epidermoid carcinoma | 0.0 |
| carcinoma | | | |
| RF-1-Gastric | 0.0 | WM266-4-Melanoma | 0.0 |
| adenocarcinoma | | | |
| RF-48-Gastric | 0.0 | DU 145-Prostate carcinoma | 0.0 |
| adenocarcinoma | | (brain metastasis) | |
| MKN-45-Gastric | 0.0 | MDA-MB-468-Breast | 6.5 |
| carcinoma | | adenocarcinoma | |
| NCI-N87-Gastric | 0.0 | SCC-4-Squamous cell | 0.0 |
| carcinoma | | carcinoma of tongue | |
| OVCAR-5-Ovarian | 10.2 | SCC-9-Squamous cell | 0.0 |
| carcinoma | | carcinoma of tongue | |
| RL95-2-Uterine | 0.0 | SCC-15-Squamous cell | 0.0 |
| carcinoma | | carcinoma of tongue | |
| HelaS3-Cervical | 0.0 | CAL 27-Squamous cell | 21.6 |
| adenocarcinoma | | carcinoma of tongue | |

TABLE MJ
Panel 4.1D

| | Rel.Exp.(%) | | Rel.Exp.(%) |
|---|---|---|---|
| | Ag3948, Run | | Ag3948, Run |
| Tissue Name | 170684837 | Tissue Name | 170684837 |
| Secondary Th1 act | 1.6 | HUVEC IL-1 beta | 3.1 |
| Secondary Th2 act | 8.6 | HUVEC IFN gamma | 7.4 |
| Secondary Tr1 act | 4.4 | HUVEC TNF alpha + IFN | 2.7 |
| | | gamma | |
| Secondary Th1 rest | 0.3 | HUVEC TNF alpha + IL4 | 0.9 |
| Secondary Th2 rest | 3.9 | HUVEC IL-11 | 6.0 |
| Secondary Tr1 rest | 1.6 | Lung Microvascular EC | 7.3 |
| | | none | |
| Primary Th1 act | 0.3 | Lung Microvascular EC | 2.7 |
| | | TNF alpha + IL-1 beta | |
| Primary Th2 act | 1.6 | 1 Microvascular Dermal EC | 0.0 |
| | | none | |
| Primary Tr1 act | 0.0 | Microsvascular Dermal EC | 0.5 |
| | | TNF alpha + IL-1 beta | |
| Primary Th1 rest | 0.0 | Bronchial epithelium | 15.5 |
| | | TNF alpha + IL1 beta | |
| Primary Th2 rest | 1.6 | Small airway epitheliurn | 6.3 |
| | | none | |
| Primary Tr1 rest | 0.4 | Small airway epithelium | 20.4 |
| | | TNF alpha + IL-1 beta | |
| CD45RA CD4 | 0.0 | Coronery artery SMC rest | 6.0 |
| lymphocyte act | | | |
| CD45RO CD4 | 1.2 | Coronery artery SMC | 1.6 |
| lymphocyte act | | TNF alpha + IL-1 beta | |
| CD8 lymphocyte act | 2.8 | Astrocytes rest | 0.3 |
| Secondary CD8 | 2.0 | Astrocytes TNF alpha + IL- | 1.5 |
| lymphocyte rest | | 1 beta | |
| Secondary CD8 | 1.2 | KU-812 (Basophil) rest | 3.4 |
| lymphocyte act | | | |
| CD4 lymphocyte none | 1.6 | KU-812 (Basophil) | 6.3 |
| | | PMA/ionomycin | |
| 2ry Th1/Th2/Tr1_anti- | 4.6 | CCD1106 (Keratinocytes) | 14.6 |
| CD95 CH11 | | none | |
| LAK cells rest | 1.6 | CCD1106 (Keratinocytes) | 20.6 |
| | | TNF alpha + IL-1 beta | |
| LAK cells IL-2 | 1.7 | Liver cirrhosis | 0.0 |
| LAK cells IL-2 + IL-12 | 3.6 | NCI-H292 none | 49.7 |
| LAK cells IL-2 + IFN | 0.0 | NCI-H292 IL-4 | 79.6 |
| gamma | | | |
| LAK cells IL-2 + IL-18 | 1.7 | NCI-H292 IL-9 | 92.7 |
| LAK cells | 0.0 | NCI-H292 IL-13 | 58.2 |
| PMA/ionomycin | | | |
| NK Cells IL-2 rest | 4.9 | NCI-H292 IFN gamma | 50.0 |
| Two Way MLR 3 day | 3.1 | HPAEC none | 8.6 |
| Two Way MLR 5 day | 1.4 | HPAEC TNF alpha + IL-1 | 3.5 |
| | | beta | |
| Two Way MLR 7 day | 4.1 | Lung fibroblast none | 2.7 |
| PBMC rest | 2.5 | Lung fibroblast TNF | 2.9 |
| | | alpha + IL-1 beta | |
| PBMC PWM | 2.3 | Lung fibroblast IL-4 | 2.9 |
| PBMC PHA-L | 0.4 | Lung fibroblast IL-9 | 4.0 |
| Ramos (B cell) none | 0.0 | Lung fibroblast IL-13 | 5.5 |
| Ramos (B cell) | 0.0 | Lung fibroblast IFN | 5.8 |
| ionomycin | | gamma | |
| B lymphocytes PWM | 0.0 | Dermal fibroblast | 16.2 |
| | | CCD1070 rest | |
| B lymphocytes CD40L | 7.6 | Dermal fibroblast | 4.7 |
| and IL-4 | | CCD1070 TNF alpha | |
| EOL-1 dbcAMP | 1.9 | Dermal fibroblast | 1.8 |
| | | CCD1070 IL-1 beta | |
| EOL-1 dbcAMP | 5.5 | Dermal fibroblast IFN | 6.7 |
| PMA/ionomycin | | gamma | |
| Dendritic cells none | 1.6 | Dermal fibroblast IL-4 | 4.1 |
| Dendritic cells LPS | 0.0 | Dermal fibroblasts rest | 12.2 |
| Dendritic cells anti- | 0.7 | Neutrophils TNFa + LPS | 0.7 |
| CD40 | | | |
| Monocytes rest | 2.8 | Neutrophils rest | 1.5 |
| Monocytes LPS | 9.3 | Colon | 5.5 |
| Macrophages rest | 0.0 | Lung | 11.6 |
| Macrophages LPS | 0.8 | Thymus | 40.6 |
| HUVEC none | 0.3 | Kidney | 100.0 |
| HUVEC starved | 9.9 | | |

TABLE MK
Panel 4D

| | Rel. | Rel. | | Rel. | Rel. |
|---|---|---|---|---|---|
| | Exp.(%) | Exp.(%) | | Exp.(%) | Exp.(%) |
| | Ag2874, | Ag3532, | | Ag2874, | Ag3532, |
| | Run | Run | | Run | Run |
| Tissue Name | 159776813 | 166444749 | Tissue Name | 159776813 | 166444749 |
| Secondary Th1 act | 0.0 | 0.0 | HUVEC IL- 1 beta | 0.0 | 0.0 |
| Secondary Th2 act | 0.0 | 0.0 | HUVEC IFN | 4.4 | 5.5 |
| | | | gamma | | |
| Secondary Tr1 act | 0.0 | 7.2 | HUVEC TNF | 0.0 | 0.0 |
| | | | alpha + IFN | | |
| | | | gamma | | |
| Secondary Th1 rest | 0.0 | 0.0 | HUVEC TNF | 0.0 | 0.0 |
| | | | alpha + IL4 | | |
| Secondary Th2 rest | 0.0 | 0.0 | HUVEC IL-11 | 0.0 | 0.0 |
| Secondary Tr1 rest | 0.0 | 3.3 | Lung | 0.0 | 9.0 |
| | | | Microvascular EC | | |
| | | | none | | |
| Primary Th1 act | 0.0 | 0.0 | Lung | 0.0 | 0.0 |
| | | | Microvascular EC | | |
| | | | TNF alpha + IL- | | |
| | | | 1 beta | | |
| Primary Th2 act | 0.0 | 0.0 | Microvascular | 0.0 | 0.0 |
| | | | Dermal EC none | | |
| Primary Tr1 act | 0.0 | 0.0 | Microsvascular | 0.0 | 0.0 |
| | | | Dermal EC | | |
| | | | TNF alpha + IL- | | |
| | | | 1 beta | | |
| Primary Th1 rest | 0.0 | 6.4 | Bronchial | 0.0 | 0.0 |
| | | | epithelium | | |
| | | | TNF alpha + | | |
| | | | IL1 beta | | |
| Primary Th2 rest | 5.6 | 0.0 | Small airway | 5.2 | 21.2 |
| | | | epithelium none | | |
| Primary Tr1 rest | 8.6 | 0.0 | Small airway | 25.5 | 19.6 |
| | | | epithelium | | |
| | | | TNF alpha + IL- | | |
| | | | 1 beta | | |
| CD45RA CD4 | 0.0 | 0.0 | Coronery artery | 0.0 | 2.1 |
| lymphocyte act | | | SMC rest | | |
| CD45RO CD4 | 0.0 | 0.0 | Coronery artery | 0.0 | 0.0 |
| lymphocyte act | | | SMC TNF alpha + | | |
| | | | IL-1 beta | | |
| CD8 lymphocyte | 0.0 | 0.0 | Astrocytes rest | 0.0 | 0.0 |
| act | | | | | |
| Secondary CD8 | 0.0 | 0.0 | Astrocytes | 0.0 | 5.8 |
| lymphocyte rest | | | TNF alpha + IL- | | |
| | | | 1 beta | | |
| Secondary CD8 | 0.0 | 0.0 | KU-812 (Basophil) | 0.0 | 0.0 |
| lymphocyte act | | | rest | | |
| CD4 lymphocyte | 4.1 | 6.9 | KU-812 (Basophil) | 0.0 | 3.0 |
| none | | | PMA/ionomycin | | |
| 2ry | 0.0 | 0.0 | CCD1106 | 13.5 | 6.7 |
| Th1/Th2/Tr1_anti- | | | (Keratinocytes) | | |
| CD95 CH11 | | | none | | |
| LAK cells rest | 0.0 | 0.0 | CCD1106 | 0.0 | 69.3 |
| | | | (Keratinocytes) | | |
| | | | TNF alpha + IL- | | |
| | | | 1 beta | | |
| LAK cells IL-2 | 0.0 | 0.0 | Liver cirrhosis | 0.0 | 0.0 |
| LAK cells IL-2 + | 0.0 | 0.0 | Lupus kidney | 0.0 | 4.2 |
| IL-12 | | | | | |
| LAK cells IL- | 0.0 | 3.5 | NCI-H292 none | 71.7 | 85.9 |
| 2 + IFN gamma | | | | | |
| LAK cells IL-2 + | 0.0 | 0.0 | NCI-H292 IL-4 | 53.6 | 100.0 |
| IL-18 | | | | | |
| LAK cells | 0.0 | 0.0 | NCI-H292 IL-9 | 100.0 | 68.8 |
| PMA/ionomycin | | | | | |
| NK Cells IL-2 rest | 0.0 | 3.4 | NCI-H292 IL-13 | 36.9 | 60.3 |
| Two Way MLR 3 | 0.0 | 0.0 | NCI-H292 IFN | 8.1 | 64.2 |
| day | | | gamma | | |
| Two Way MLR 5 | 0.0 | 7.0 | HPAEC none | 0.0 | 0.0 |
| day | | | | | |
| Two Way MLR 7 | 0.0 | 0.9 | HPAEC TNF | 0.0 | 0.0 |
| day | | | alpha + IL-1 beta | | |
| PBMC rest | 0.0 | 0.0 | Lung fibroblast | 0.0 | 0.0 |
| | | | none | | |
| PBMC PWM | 8.2 | 6.9 | Lung fibroblast | 4.0 | 4.9 |
| | | | TNF alpha + IL-1 | | |
| | | | beta | | |
| PBMC PHA-L | 0.0 | 0.0 | Lung fibroblast IL-4 | 0.0 | 0.0 |
| Ramos (B cell) | 0.0 | 0.0 | Lung fibroblast IL-9 | 4.6 | 0.0 |
| none | | | | | |
| Ramos (B cell) | 0.0 | 0.0 | Lung fibroblast IL- | 0.0 | 0.0 |
| ionomycin | | | 13 | | |
| B lymphocytes | 0.0 | 3.4 | Lung fibroblast | 5.0 | 0.0 |
| PWM | | | IFN gamma | | |
| B lymphocytes | 0.0 | 5.0 | Dermal fibroblast | 4.6 | 3.0 |
| CD40L and IL-4 | | | CCD1070 rest | | |
| EOL-1 dbcAMP | 0.0 | 0.0 | Dermal fibroblast | 0.0 | 0.0 |
| | | | CCD1070 TNF | | |
| | | | alpha | | |
| EOL-1 dbcAMP | 3.7 | 0.0 | Dermal fibroblast | 0.0 | 0.0 |
| PMA/ionomycin | | | CCD1070 IL-1 | | |
| | | | beta | | |
| Dendritic cells none | 0.0 | 0.0 | Dermal fibroblast | 4.6 | 6.6 |
| | | | IFN gamma | | |
| Dendritic cells LPS | 0.0 | 0.0 | Dermal fibroblast | 0.0 | 6.5 |
| | | | IL-4 | | |
| Dendritic cells anti- | 0.0 | 2.1 | IBD Colitis 2 | 0.0 | 0.0 |
| CD40 | | | | | |
| Monocytes rest | 4.5 | 0.0 | IBD Crohn's | 0.0 | 0.0 |
| Monocytes LPS | 8.6 | 2.7 | Colon | 12.0 | 13.7 |
| Macrophages rest | 0.0 | 0.0 | Lung | 0.0 | 0.0 |
| Macrophages LPS | 0.0 | 0.0 | Thymus | 0.0 | 7.6 |
| HUVEC none | 0.0 | 0.0 | Kidney | 4.0 | 17.6 |
| HUVEC starved | 0.0 | 3.5 | | | |

AI_comprehensive panel_v1.0 Summary: Ag3948 This panel confirms expression of the CG59293-01 gene in tissue samples related to the immune and inflammatory response. Please see Panels 4 and 4.1D for discussion of utility of this gene in inflammation.

CNS_neurodegeneration_v1.0 Summary: Ag3948 This panel does not show differential expression of the CG59293-01 gene in Alzheimer's disease. However, this expression profile confirms the presence of this gene in the brain. Please see Panel General_screening_panel_v1.4 for discussion of utility of this gene in the central nervous system. Ag3532 Expression of the CG59293-01 gene is low/undetectable in all samples on this panel (CTs>35).

General_screening_panel_v1.4 Summary: Ag3948 The expression of the CG59293-01 gene, an organic anion transporter homolog, is highest in a small cell lung cancer line LX-1 (CT-28.2). This gene is also expressed in some ovarian, breast, CNS, gastric. pancreatic, renal and colon cancer cell lines. Therefore, expression of this gene may be associated with these forms of cancer and therapeutic modulation of this gene might be of use in the treatment or diagnosis of these cancers.

This gene is also expressed at low levels in the cerebellum and fetal brain. The organic anion transporters are involved in transport across the blood brain barrier. This gene may therefore be of use in drug delivery to the CNS, specifically for compounds such as nerve growth factors protein therapeutics which are believed to have numerous uses in the CNS, but lack a delivery system.

References:

Sugiyama D, Kusuhara H, Shitara Y, Abe T, Meier P J, Sekine T, Endou H, Suzuki H, Sugiyama Y. Characterization of the efflux transport of 17beta-estradiol-D-17beta-glucuronide from the brain across the blood-brain barrier.

Panel 1.3D Summary: Ag2874 The expression of the CG59293-01 gene was assessed in two independent runs on this panel with reasonable concordance between the runs. The highest expression is seen in a small cell lung cancer line LX-1 (CTs=3 1-32), consistent with expression in Panel 1.3D. This gene is also expressed in some ovarian, breast, CNS, gastric and colon cancer cell lines. Therefore, expression of this gene might be associated with these forms of cancer and therapeutic modulation of this gene might be of use in the treatment or diagnosis of these cancers.

Panel 2D Summary: Ag2874 The CG59293-01 gene is expressed at low levels in the tissues used for panel 2D. The highest expression is seen in a breast cancer sample (CT=34.2). Significant expression is also seen in single samples of ovarian, bladder, prostate and colon cancers compared with the normal adjacent tissue. This indicates that the expression of this gene might be associated with these forms of cancer and therapeutic modulation of this gene might be of use in the treatment or diagnosis of these cancers.

Panel 3D Summary: Ag2874 Highest expression of the CG59293-01 gene is seen in a pancreatic cancer cell line (CT=31.6). Thus, expression of this gene could be used to differentiate between this sample and other samples on this panel. In addition, significant expression of this gene is associated with samples derived from lung cancer cell lines (CTs=32-34), an ovarian cancer, gastric cancer and squamous cell carcinoma of tongue. Therefore, therapeutic modulation of the activity of this gene or its protein product, through the use of small molecule drugs, protein therapeutics or antibodies, might be beneficial in the treatment of these cancers.

Panel 4D Summary: Ag3498 The highest expression of the CG59293-01 gene is found in the kidney and in the pulmonary muco-epidermoid cell line NCI-H292 (CTs=31). The expression of this gene, although constitutive in the H292 cell line, is up regulated upon treatment with IL-4, II-9 and IL-13, cytokines that have been linked to the pathogenesis of asthma and/or COPD.

This gene is also found in small airway epithelium and keratinocytes treated with the inflammatory cytokines TNF-a and IL-1b. Therefore, modulation of the expression or activity of the protein encoded by this transcript through the application or small molecule therapeutics may be useful in the treatment of asthma, COPD, emphysema, psoriasis and wound healing.

Panel 4D Summary: Ag2874/Ag3532 The expression of the CG59293-01 transcript detected by two sets of primers was observed mostly in NCI-H292 (CFs=32-33). Up-regulation of this transcript in H292 was found upon treatment with IL-4 (Ag 3532) or IL-9 (Ag 2874), both cytokines are involved in the pathogenesis of asthma and/or COPD. This transcript was also detected in small airway epithelium and keratinocytes. Therefore, modulation of the expression or activity of the protein encoded by this transcript by small molecules could be useful for the treatment of asthma, COPD and or emphysema and also for the treatment of skin inflammatory diseases such as psoriasis or wound healing.

### N. NOV19a (CG59284-01: SOLUTE CARRIER FAMILY 22)

Expression of gene CG59284-01 was assessed using the primer-probe set Ag3528, described in Table NA.

TABLE NA
Probe Name Ag3528

| | | | | Start |
|---|---|---|---|---|
| Primers | Sequences | | Length | Position |
| Forward | 5′-gtgtggatggctggqtctat-3′ | (SEQ ID NO:263) | 20 | 437 |
| Probe | TET-5′-cagcatcttcacctccacaatcgtg-3′-TAMRA | (SEQ ID NO:264) | 25 | 462 |
| Revers | 5′-ttcagagcatgagagtcacaca-3′ | (SEQ ID NO:265) | 22 | 503 |

CNS_neurodegeneration_v1.0 Summary: Ag3528 Expression of the CG59284-01 gene is low/undetectable in all samples on this panel (CTs>35).

General_screen_panel_v1.4 Summary: Ag3528 Expression of the CG59284-01 gene is low/undetectable in all samples on this panel (CTs>35).

Panel 4.1D Summary: Ag3528 Expression of the CG59284-01 gene is low/undetectable in all samples on this panel (CTs>35).

### O. NOV20a (CG59278-01: OLFACTORY RECEPTOR P2)

Expression of gene CG59278-01 was assessed using the primer-probe set Ag3526, described in Table OA. Results of the RTQ-PCR runs are shown in Table OB.

TABLE OA
Probe Name Ag3526

| | | | | Start |
|---|---|---|---|---|
| Primers | Sequences | | Length | Position |
| Forward | 5′-gtagtgccaaggccttctctac-3′ | (SEQ ID NO:266) | 22 | 738 |
| Probe | TET-5′-ctcccacgtaactgtggtggcactat-3′-TAMRA | (SEQ ID NO:267) | 26 | 766 |
| Reverse | 5′-actgtgaaggcagaagcataga-3′ | (SEQ ID NO:268) | 22 | 792 |

TABLE OB
Panel 4.1D

| | Rel. Exp.(%) | | Rel. Exp.(%) |
|---|---|---|---|
| | Ag3526, Run | | Ag3526, Run |
| Tissue Name | 169840752 | Tissue Name | 169840752 |
| Secondary Th1 act | 0.0 | HUVEC IL-1 beta | 0.0 |
| Secondary Th2 act | 0.0 | HUVEC IFN gamma | 0.0 |
| Secondary Tr1 act | 0.5 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1 beta | |
| Primary Th2 act | 0.3 | Microvascular Dermal EC | 0.0 |
| | | none | |
| Primary Tr1 act | 0.0 | Microsvasular Dermal EC | 0.0 |
| | | TNF alpha + IL-1 beta | |
| Primary Th1 rest | 0.0 | Bronchial epithelium | 0.0 |
| | | TNF alpha + IL1 beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1 beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1 beta | |
| CD8 lymphocyte act | 0.0 | Astrocytes rest | 0.0 |
| Secondary CD8 | 0.0 | Astrocytes TNF alpha + IL- | 0.0 |
| lymphocyte rest | | 1 beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1 beta | |
| LAK cells IL-2 | 0.0 | Liver cirrhosis | 0.0 |
| LAK cells IL-2 + IL-12 | 0.0 | NCI-H292 none | 0.0 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 0.0 |
| LAK cells | 0.4 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL-1 | 0.0 |
| | | beta | |
| Two Way MLR 7 day | 0.0 | Lung fibroblast none | 0.0 |
| PBMC rest | 0.0 | Lung fibroblast TNF | 0.0 |
| | | alpha + IL-1 beta | |
| PBMC PWM | 0.0 | Lung fibroblast IL-4 | 0.8 |
| PBMC PHA-L | 0.0 | Lung fibroblast IL-9 | 0.0 |
| Ramos (B cell) none | 0.0 | Lung fibroblast IL-13 | 0.4 |
| Ramos (B cell) | 0.0 | Lung fibroblast IFN | 0.0 |
| ionomycin | | gamma | |
| B lymphocytes PWM | 0.0 | Dermal fibroblast | 0.0 |
| | | CCD1070 rest | |
| B lymphocytes CD40L | 0.0 | Dermal fibroblast | 0.0 |
| and IL-4 | | CCD1070 TNF alpha | |
| EOL-1 dbcAMP | 0.0 | Dermal fibroblast | 0.0 |
| | | CCD1070 IL-1 beta | |
| EOL-1 dbcAMP | 0.0 | Dermal fibroblast IFN | 0.0 |
| PMA/ionomycin | | gamma | |
| Dendritic cells none | 0.0 | Dermal fibroblast IL-4 | 0.0 |
| Dendritic cells LPS | 0.6 | Dermal Fibroblasts rest | 0.0 |
| Dendritic cells anti- | 0.3 | Neutrophils TNFa + LPS | 0.4 |
| CD40 | | | |
| Monocytes rest | 7.0 | Neutrophils rest | 100.0 |
| Monocytes LPS | 5.6 | Colon | 0.0 |
| Macrophages rest | 0.7 | Lung | 0.4 |
| Macrophages LPS | 0.0 | Thymus | 0.7 |
| HUVEC none | 0.0 | Kidney | 0.0 |
| HUVEC starved | 0.0 | | |

CNS_neurodegeneration_v1.0 Summary: Ag3526 Expression of the CG59278-01 gene is low/undetectable in all samples on this panel (CTs>35).

General_screening_panel_v1.4 Summary: Ag3526 Expression of the CG59278-01 gene is low/undetectable in all samples on this panel (CTs>35). (Data not shown). This gene encodes a G protein-coupled receptor (GPCR), a type of cell surface receptor involved in signal transduction. This gene product is most similar to members of the odorant receptor subfamily of GPCRs. Based on analogy to other odorant receptor genes, we predict that expression of this gene may be highest in nasal epithelium, a sample not represented on this panel.

Panel 4.1 D Summary: Ag3526 Expression of the CG59278-01 gene is restricted to a sample derived from resting neutrophils (CT=28.2). Thus, expression of this gene could be used as a marker of these cells. This expression is markedly reduced (CT=36.3) in neutrophils activated by TNF-alpha+LPS. This expression profile suggest that the protein encoded by this gene is produced by resting neutrophils but not by activated neutrophils. Therefore, the gene product may reduce activation of these inflammatory cells and be useful as a protein therapeutic to reduce or eliminate the symptoms in patients with Crohn's disease, ulcerative colitis, multiple sclerosis, chronic obstructive pulmonary disease, asthma, emphysema, rheumatoid arthritis, lupus erythematosus, or psoriasis. In addition, small molecule or antibody antagonists of this gene product may be effective in increasing the immune response in patients with AIDS or other immunodeficiencies.

### P. NOV21a and NOV21b (CG59274-01 and CG59274-02: LIPOMA HMGIC FUSION PARTNER)

Expression of gene CG59274-01 and CG59274-02 was assessed using the primer-probe set Ag3525, described in Table PA. Results of the RTQ-PCR runs are shown in Tables PB, PC and PD. Please note that CG59274-02 represents a full-length physical clone of the CG59274-01 gene, validating the prediction of the gene sequence.

TABLE PA
Probe Name Ag3525

| | | | | Start |
|---|---|---|---|---|
| Primer | Sequences | | Length | Position |
| Forward | 5′-gagagcatcatgaggaatacca-3′ | (SEQ ID NO:269) | 22 | 664 |
| Probe | TET-5′-accattgcctcaaaccttgagctttg-3′-TAMRA | (SEQ ID NO:270) | 26 | 707 |
| Reverse | 5′-ccaccctctccaatcttcttt-3′ | (SEQ ID NO:271) | 21 | 733 |

TABLE PB
CNS_neurodegeneration_v1.0

| | Rel. Exp.(%) Ag3525, | | Rel. Exp.(%) Ag3525, |
|---|---|---|---|
| Tissue Name | Run 210630172 | Tissue Name | Run 210630172 |
| AD 1 Hippo | 8.7 | Control (Path) 3 | 8.1 |
| | | Temporal Ctx | |
| AD 2 Hippo | 50.0 | Control (Path) 4 | 39.2 |
| | | Temporal Ctx | |
| AD 3 Hippo | 4.2 | AD 1 Occipital Ctx | 7.6 |
| AD 4 Hippo | 10.4 | AD 2 Occipital Ctx | 0.0 |
| | | (Missing) | |
| AD 5 hippo | 36.3 | AD 3 Occipital Ctx | 1.1 |
| AD 6 Hippo | 100.0 | AD 4 Occipital Ctx | 15.4 |
| Control 2 Hippo | 28.5 | AD 5 Occipital Ctx | 7.9 |
| Control 4 Hippo | 31.9 | AD 6 Occipital Ctx | 18.0 |
| Control (Path) 3 | 7.2 | Control 1 Occipital | 0.0 |
| Hippo | | Ctx | |
| AD 1 Temporal Ctx | 18.4 | Control 2 Occipital | 20.7 |
| | | Ctx | |
| AD 2 Temporal Ctx | 39.0 | Control 3 Occipital | 6.0 |
| | | Ctx | |
| AD 3 Temporal Ctx | 3.0 | Control 4 Occipital | 16.6 |
| | | Ctx | |
| AD 4 Temporal Ctx | 26.6 | Control (Path) 1 | 49.0 |
| | | Occipital Ctx | |
| AD 5 Inf Temporal | 40.9 | Control (Path) 2 | 6.4 |
| Ctx | | Occipital Ctx | |
| AD 5 SupTemporal | 60.7 | Control (Path) 3 | 3.1 |
| Ctx | | Occipital Ctx | |
| AD 6 Inf Temporal | 50.3 | Control (Path) 4 | 4.5 |
| Ctx | | Occipital Ctx | |
| AD 6 Sup Temporal | 49.0 | Control 1 Parietal | 0.6 |
| Ctx | | Ctx | |
| Control 1 Temporal | 0.6 | Control 2 Parietal | 30.6 |
| Ctx | | Ctx | |
| Control 2 Temporal | 22.1 | Control 3 Parietal | 5.3 |
| Ctx | | Ctx | |
| Control 3 Temporal | 12.2 | Control (Path) 1 | 30.6 |
| Ctx | | Parietal Ctx | |
| Control 4 Temporal | 28.9 | Control (Path) 2 | 23.8 |
| Ctx | | Parietal Ctx | |
| Control (Path) 1 | 30.6 | Control (Path) 3 | 2.6 |
| Temporal Ctx | | Parietal Ctx | |
| Control (Path) 2 | 23.7 | Control (Path) 4 | 19.8 |
| Temporal Ctx | | Parietal Ctx | |

TABLE PC
General_screening_panel_v1.4

| | Rel. Exp.(%) Ag3525, | | Rel. Exp.(%) Ag3525, |
|---|---|---|---|
| Tissue Name | Run 220356041 | Tissue Name | Run 220356041 |
| Adipose | 1.9 | Renal ca. TK-10 | 0.7 |
| Melanoma* | 0.0 | Bladder | 0.6 |
| Hs688(A).T | | | |
| Melanoma* | 0.0 | Gastric ca. (liver met.) | 1.9 |
| Hs688(B).T | | NCI-N87 | |
| Melanoma* M14 | 0.7 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.6 |
| MEL-5 | | | |
| Squamous cell | 0.7 | Colon ca.* (SW480 | 0.0 |
| carcinoma SCC-4 | | met) SW620 | |
| Testis Pool | 2.6 | Colon ca. HT29 | 0.0 |
| Prostate ca.* (bone | 0.0 | Colon ca. HCT-116 | 0.0 |
| met) PC-3 | | | |
| Prostate Pool | 5.3 | Colon ca. CaCo-2 | 0.7 |
| Placenta | 1.2 | Colon cancer tissue | 0.0 |
| Uterus Pool | 0.9 | Colon ca. SW1116 | 0.0 |
| Ovarian ca. OVCAR-3 | 11.9 | Colon ca.Colo-205 | 0.0 |
| Ovarian ca. SK-OV-3 | 0.0 | Colon ca. SW-48 | 0.0 |
| Ovarian ca. OVCAR-4 | 0.0 | Colon Pool | 3.6 |
| Ovarian ca. OVCAR-5 | 0.0 | Small Intestine Pool | 4.2 |
| Ovarian ca. IGROV-1 | 1.4 | Stomach Pool | 0.4 |
| Ovarian ca. OVCAR-8 | 2.0 | Bone Marrow Pool | 1.7 |
| Ovary | 8.5 | Fetal Heart | 4.2 |
| Breast ca. MCF-7 | 1.8 | Heart Pool | 3.1 |
| Breast ca. MDA- | 0.0 | Lymph Node Pool | 2.7 |
| MB-231 | | | |
| Breast ca. BT 549 | 1.9 | Fetal Skeletal Muscle | 0.5 |
| Breast ca. T47D | 1.1 | Skeletal Muscle Pool | 5.4 |
| Breast ca. MDA-N | 2.7 | Spleen Pool | 6.7 |
| Breast Pool | 0.7 | Thymus Pool | 9.9 |
| Trachea | 39.8 | CNS cancer (glio/astro) | 0.0 |
| | | U87-MG | |
| Lung | 5.0 | CNS cancer (glio/astro) | 0.0 |
| | | U-118-MG | |
| Fetal Lung | 13.7 | CNS cancer (neuro; met) | 0.6 |
| | | SK-N-AS | |
| Lung ca. NCI-N417 | 0.0 | CNS cancer (astro) SF- | 0.0 |
| | | 539 | |
| Lung ca. LX-1 | 0.0 | CNS cancer (astro) | 4.6 |
| | | SNB-75 | |
| Lung ca. NCI-H146 | 4.8 | CNS cancer (glio) SNB-19 | 0.7 |
| Lung ca. SHP-77 | 0.0 | CNS cancer (glio) SF-295 | 5.9 |
| Lung ca. A549 | 0.0 | Brain (Amygdala) Pool | 46.3 |
| Lung ca. NCI-H526 | 0.0 | Brain (cerebellum) | 4.6 |
| Lung ca. NCI-H23 | 1.1 | Brain (fetal) | 6.4 |
| Lung ca. NCI-H460 | 0.0 | Brain (Hippocampus) | 70.2 |
| | | Pool | |
| Lung ca.HOP-62 | 0.0 | Cebrebral Cortex Pool | 49.7 |
| Lung ca. NCI-H522 | 0.0 | Brain (Substantia nigra) | 23.5 |
| | | Pool | |
| Liver | 0.0 | Brain (Thalamus) Pool | 100.0 |
| Fetal Liver | 1.3 | Brain (whole) | 21.8 |
| Liver ca. HepG2 | 0.0 | Spinal Cord Pool | 18.4 |
| Kidney Pool | 8.4 | Adrenal Gland | 3.1 |
| Fetal Kidney | 8.5 | Pituitary gland Pool | 50.3 |
| Renal ca. 786-0 | 0.0 | Salivary Gland | 7.7 |
| Renal ca. A498 | 0.0 | Thyroid (female) | 17.0 |
| Renal ca. ACHN | 0.0 | Pancreatic ca. CAPAN2 | 0.0 |
| Renal ca. UO-31 | 0.4 | Pancreas Pool | 0.0 |

TABLE PD
Panel 4D

| | Rel. Exp.(%) | | Rel. Exp.(%) |
|---|---|---|---|
| | Ag3525, Run | | Ag3525, Run |
| Tissue Name | 166446262 | Tissue Name | 166446262 |
| Secondary Th1 act | 0.0 | HUVEC IL-1 beta | 0.0 |
| Secondary Th2 act | 6.6 | HUVEC IFN gamma | 0.0 |
| Secondary Tr1 act | 4.5 | HUVEC TNF alpha + IFN | 0.0 |
| | | gamma | |
| Secondary Th1 rest | 0.0 | HUVEC TNF alpha + IL4 | 0.0 |
| Secondary Th2 rest | 3.3 | HUVEC IL-11 | 0.0 |
| Secondary Tr1 rest | 6.7 | Lung Microvasular EC | 8.5 |
| | | none | |
| Primary Th1 act | 0.0 | Lung Microvascular EC | 20.4 |
| | | TNF alpha + IL-1 beta | |
| Primary Th2 act | 6.3 | Microvascular Dermal EC | 0.0 |
| | | none | |
| Primary Tr1 act | 6.7 | Microsvascular Dermal EC | 0.0 |
| | | TNF alpha + IL-1 beta | |
| Primary Th1 rest | 33.4 | Bronchial epithelium | 0.0 |
| | | TNF alpha + IL-1 beta | |
| Primary Th2 rest | 18.4 | Small airway epithelium | 0.0 |
| | | none | |
| Primary Tr1 rest | 14.4 | Small airway epithelium | 3.0 |
| | | TNF alpha + IL-1 beta | |
| CD45RA CD4 | 6.0 | Coronery artery SMC rest | 9.7 |
| lymphocyte act | | | |
| CD45RO CD4 | 5.1 | Coronery artery SMC | 10.8 |
| lymphocyte act | | TNF alpha + IL-1 beta | |
| CD8 lymphocyte act | 21.6 | Astrocytes rest | 3.6 |
| Secondary CD8 | 3.3 | Astrocytes TNF alpha + IL- | 30.4 |
| lymphocyte rest | | 1 beta | |
| Secondary CD8 | 5.1 | KU-812 (Basophil) rest | 0.0 |
| lymphocyte act | | | |
| CD4 lymphocyte none | 2.7 | KU-812 (Basophil) | 0.0 |
| | | PMA/ionomycin | |
| 2ry Th1/Th2/Tr1_anti- | 13.2 | CCD1106 (Keratinocytes) | 0.0 |
| CD95 CH11 | | none | |
| LAK cells rest | 3.6 | CCD1106 (Keratinocytes) | 3.3 |
| | | TNF alpha + IL-1 beta | |
| LAK cells IL-2 | 45.7 | Liver cirrhosis | 83.5 |
| LAK cells IL-2 + IL-12 | 46.7 | Lupus kidney | 4.6 |
| LAK cells IL-2 + IFN | 40.1 | NCI-H292 none | 0.0 |
| gamma | | | |
| LAK cells IL-2 + IL-18 | 20.4 | NCI-H292 IL-4 | 0.0 |
| LAK cells | 7.5 | NCI-H292 IL-9 | 0.0 |
| PMA/ionomycin | | | |
| NK Cells IL-2 rest | 8.0 | NCI-H292 IL-13 | 0.0 |
| Two Way MLR 3 day | 100.0 | NCI-H292 IFN gamma | 3.8 |
| Two Way MLR 5 day | 13.7 | HPAEC none | 35.4 |
| Two Way MLR 7 day | 10.7 | HPAEC TNF alpha + IL-1 | 3.0 |
| | | beta | |
| PBMC rest | 6.6 | Lung fibroblast none | 3.4 |
| PBMC PWM | 9.4 | Lung fibroblast TNF | 0.0 |
| | | alpha + IL-1 beta | |
| PBMC PHA-L | 0.0 | Lung fibroblast IL-4 | 6.8 |
| Ramos (B cell) none | 0.0 | Lung fibroblast IL-9 | 0.0 |
| Ramos (B cell) | 0.0 | Lung fibroblast IL-13 | 3.3 |
| ionomycin | | | |
| B lymphocytes PWM | 54.3 | Lung fibroblast IFN | 89.5 |
| | | gamma | |
| B lymphocytes CD40L | 9.7 | Dermal fibroblast | 6.0 |
| and IL-4 | | CCD1070 rest | |
| EOL-1 dbcAMP | 0.0 | Dermal fibrobaslat | 4.2 |
| | | CCD1070 TNF alpha | |
| EOL-1 dbcAMP | 0.0 | Dermal fibroblast | 0.0 |
| PMA/ionomycin | | CCD1070 IL-1 beta | |
| Dendritic cells none | 3.3 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 3.2 |
| CD40 | | | |
| Monocytes rest | 0.0 | IBD Crohn's | 18.6 |
| Monocytes LPS | 0.0 | Colon | 72.2 |
| Macrophages rest | 4.2 | Lung | 9.7 |
| Macrophages LPS | 0.0 | Thymus | 37.6 |
| HUVEC none | 0.0 | Kidney | 62.9 |
| HUVEC starved | 0.0 | | |

CNS_neurodegeneration_v1.0 Summary: Ag3525 This panel does not show differential expression of the CG59274-01 gene in Alzheimer's disease. However, this expression profile confirms the presence of this gene in the brain. Please see Panel 1.4 for discussion of utility of this gene in the central nervous system.

General_screening_panel_v1.4 Summary: Ag3525 The CG59274-01 gene exhibits highly brain preferential expression, with moderate to low levels of expression seen in all regions of the brain examined in this panel. Thus, therapeutic modulation of the expression or function of this gene may be effective in the treatment of neurologic disorders, including Alzheimer's disease, Parkinson's disease, epilepsy, stroke and schizophrenia.

Among metabolic tissues, expression of this gene is limited to the thyroid. Thus, this gene product may also be effective in treating thyroidopathies.

Panel 4D Summary: Ag3525 The CG59274-01 gene is expressed in normal kidney, thymus and colon as well as in activated LAK cells(CTs=-32-33). The gene is also expressed at lower but still significant levels in primary resting T cells, with highest expression seen in a sample derived from an MLR reaction (CT=32). Thus, the protein encoded by this transcript could be important in the function of LAK cells. LAK cells are important in immunosurveillance against bacterial and viral infected cells, as well as transformed cells. Therapeutics designed with this transcript or the protein encoded by it could be important in the treatment of viral and bacterial diseases and cancer.

### Q. NOV23a and NOV23b (CG57734-01 and CG57734-02: LIPID ASSOCIATED PROTEIN)

Expression of gene CG57734-01 and CG57734-01 was assessed using the primer-probe set Ag572, described in Table QA. Please note that CG57734-02 represents a full-length physical clone of the CG57734-01 gene, validating the prediction of the gene sequence.

TABLE QA
Probe Name Ag572

| | | | | Start |
|---|---|---|---|---|
| Primers | Sequences | | Length | Position |
| Forward | 5′-ctgcgcttcgatgtgtacaac-3′ | (SEQ ID NO:272) | 21 | 431 |
| Probe | TET-5′-actccaaaaccaacatctccaaaccgaag-3′-TAMRA | (SEQ ID NO:273) | 29 | 456 |
| Reverse | 5′-ggaacgcttgtcccaggaa-3′ | (SEQ ID NO:274) | 19 | 488 |

Panel 1.1 Summary: Ag5734 Expression of the CG57734-01 gene is low/undetectable in all samples on this panel (CTs>35).

### R. NOV25a (CG59885-01: HGFR)

Expression of gene CG59885-01 was assessed using the primer-probe set Ag1684, described in Table RA. Results of the RTQ-PCR runs are shown in Tables RB, RC, RD, RE, RF, and RG.

TABLE RA
Probe Name Ag1684

| | | | | Start |
|---|---|---|---|---|
| Primer | Sequences | | Length | Position |
| Forward | 5′-tgaagcaggaaggaactttaca-3′ | (SEQ ID NO:275) | 22 | 2582 |
| Probe | TET-5′-tggcatgtcaacatcgctctaattca-3′-TAMRA | (SEQ ID NO:276) | 26 | 2605 |
| Reverse | 5′-gggaaggagtggtacaacaga-3′ | (SEQ ID NO:277) | 21 | 2638 |

TABLE RB
CNS_neurodegeneration_v1.0

| | Rel. Exp.(%) Ag1684, | | Rel. Exp.(%), Ag1684, |
|---|---|---|---|
| Tissue Name | Run 207624860 | Tissue Name | Run 207624860 |
| AD 1 Hippo | 1.7 | Control (Path) 3 | 2.3 |
| | | Temporal Ctx | |
| AD 2 Hippo | 9.7 | Control (Path) 4 | 8.2 |
| | | Temporal Ctx | |
| AD 3 Hippo | 6.2 | AD 1 Occipital Ctx | 7.9 |
| AD 4 Hippo | 3.1 | AD 2 Occipital Ctx | 0.0 |
| | | (Missing) | |
| AD 5 hippo | 100.0 | AD 3 Occipital Ctx | 1.7 |
| AD 6 Hippo | 14.1 | AD 4 Occipital Ctx | 11.3 |
| Control 2 Hippo | 15.2 | AD 5 Occipital Ctx | 7.5 |
| Control 4 Hippo | 2.0 | AD 6 Occipital Ctx | 24.7 |
| Control (Path) 3 | 3.3 | Control 1 Occipital | 2.0 |
| Hippo | | Ctx | |
| AD 1 Temporal Ctx | 1.1 | Control 2 Occipital | 76.3 |
| | | Ctx | |
| AD 2 Temporal Ctx | 9.0 | Control 3 Occipital | 6.7 |
| | | Ctx | |
| AD 3 Temporal Ctx | 1.8 | Control 4 Occipital | 2.8 |
| | | Ctx | |
| AD 4 Temporal Ctx | 7.2 | Control (Path) 1 | 60.7 |
| | | Occipital Ctx | |
| AD 5 Inf Temporal | 53.6 | Control (Path) 2 | 6.7 |
| Ctx | | Occipital Ctx | |
| AD 5 SupTemporal | 18.4 | Control (Path) 3 | 1.7 |
| Ctx | | Occipital Ctx | |
| AD 6 Inf Temporal | 10.8 | Control (Path) 4 | 6.8 |
| Ctx | | Occipital Ctx | |
| AD 6 Sup Temporal | 10.5 | Control 1 Parietal | 1.5 |
| Ctx | | Ctx | |
| Control 1 Temporal | 1.2 | Control 2 Parietal | 11.0 |
| Ctx | | Ctx | |
| Control 2 Temporal | 29.9 | Control 3 Parietal | 3.3 |
| Ctx | | Ctx | |
| Control 3 Temporal | 3.3 | Control (Path) 1 | 37.9 |
| Ctx | | Parietal Ctx | |
| Control 4 Temporal | 2.0 | Control (Path) 2 | 6.4 |
| Ctx | | Parietal Ctx | |
| Control (Path) 1 | 28.1 | Control (Path) 3 | 0.4 |
| Temporal Ctx | | Parietal Ctx | |
| Control (Path) 2 | 12.2 | Control (Path) 4 | 10.0 |
| Temporal Ctx | | Parietal Ctx | |

TABLE RC
General_screening_panel_v1.4

| | Rel. Exp.(%) Ag1684, | | Rel. Exp.(%) Ag1684, |
|---|---|---|---|
| Tissue Name | Run 208022075 | Tissue Name | Run 208022075 |
| Adipose | 3.0 | Renal ca. TK-10 | 23.2 |
| Melanoma* | 5.4 | Bladder | 3.9 |
| Hs688(A).T | | | |
| Melanoma* | 5.8 | Gastric ca. (liver met.) | 27.9 |
| Hs688(B).T | | NCI-N87 | |
| Melanoma* M14 | 0.0 | Gastric ca. KATO III | 67.4 |
| Melanoma* | 10.2 | Colon ca. SW-948 | 8.9 |
| LOXIMVI | | | |
| Melanoma* SK- | 80.7 | Colon ca. SW480 | 0.6 |
| MEL-5 | | | |
| Squamous cell | 15.2 | Colon ca.* (SW480 | 9.6 |
| carcinoma SCC-4 | | met) SW620 | |
| Testis Pool | 0.2 | Colon ca. HT29 | 14.7 |
| Prostate ca.* (bone | 37.6 | Colon ca. HCT-116 | 36.6 |
| met) PC-3 | | | |
| Prostate Pool | 0.5 | Colon ca. CaCo-2 | 23.5 |
| Placenta | 1.2 | Colon cancer tissue | 9.1 |
| Uterus Pool | 0.5 | Colon ca. SW1116 | 1.7 |
| Ovarian ca. OVCAR-3 | 5.1 | Colon ca. Colo-205 | 2.0 |
| Ovarian ca. SK-OV-3 | 14.8 | Colon ca. SW-48 | 2.3 |
| Ovarian ca. OVCAR-4 | 7.6 | Colon Pool | 0.9 |
| Ovarian ca. OVCAR-5 | 29.9 | Small Intestine Pool | 0.5 |
| Ovarian ca. IGROV-1 | 9.1 | Stomach Pool | 0.9 |
| Ovarian ca. OVCAR-8 | 2.6 | Bone Marrow Pool | 0.4 |
| Ovary | 0.4 | Fetal Heart | 0.2 |
| Breast ca. MCF-7 | 2.5 | Heart Pool | 0.5 |
| Breast ca. MDA- | 14.5 | Lymph Node Pool | 1.1 |
| MB-231 | | | |
| Breast ca. BT 549 | 3.7 | Fetal Skeletal Muscle | 0.4 |
| Breast ca. T47D | 29.3 | Skeletal Muscle Pool | 1.9 |
| Breast ca. MDA-N | 15.4 | Spleen Pool | 1.7 |
| Breast Pool | 1.1 | Thymus Pool | 0.7 |
| Trachea | 1.7 | CNS cancer (glio/astro) | 25.7 |
| | | U87-MG | |
| Lung | 0.2 | CNS cancer (glio/astro) | 38.2 |
| | | U-118-MG | |
| Fetal Lung | 13.5 | CNS cancer (neuro; met) | 0.0 |
| | | SK-N-AS | |
| Lung ca. NCI-N417 | 1.1 | CNS cancer (astro) SF-539 | 2.2 |
| Lung ca. LX-1 | 16.6 | CNS cancer (astro) SNB-75 | 12.6 |
| Lung ca. NCI-H146 | 1.2 | CNS cancer (glio) SNB-19 | 7.8 |
| Lung ca. SHP-77 | 3.0 | CNS cancer (glio) SF-295 | 19.1 |
| Lung ca. A549 | 23.8 | Brain (Amygdala) Pool | 0.3 |
| Lung ca. NCI-H526 | 2.8 | Brain (cerebellum) | 0.0 |
| Lung ca. NCI-H23 | 7.7 | Brain (fetal) | 1.7 |
| Lung ca. NCI-H460 | 4.8 | Brain (Hippocampus) Pool | 0.5 |
| Lung ca. HOP-62 | 11.7 | Cerebral Cortex Pool | 1.0 |
| Lung ca. NCI-H522 | 0.1 | Brain (Substantia nigra) | 0.2 |
| | | Pool | |
| Liver | 1.5 | Brain (Thalamus) Pool | 1.6 |
| Fetal Liver | 8.8 | Brain (whole) | 2.1 |
| Liver ca. HepG2 | 7.7 | Spinal Cord Pool | 0.2 |
| Kidney Pool | 1.2 | Adrenal Gland | 0.1 |
| Fetal Kidney | 2.6 | Pituitary gland Pool | 0.6 |
| Renal ca. 786-0 | 26.1 | Salivary Gland | 0.3 |
| Renal ca. A498 | 19.1 | Thyroid (female) | 3.3 |
| Renal ca. ACHN | 20.4 | Pancreatic ca. CAPAN2 | 59.0 |
| Renal ca. UO-31 | 100.0 | Pancreas Pool | 5.0 |

TABLE RD
Panel 1.3D

| | Rel. Exp.(%) | Rel. Exp.(%) | | Rel. Exp.(%) | Rel. Exp.(%) |
|---|---|---|---|---|---|
| | Ag1684, Run | Ag1684, Run | | Ag1684, Run | Ag1684, Run |
| Tissue Name | 158401632 | 165526993 | Tissue Name | 158401632 | 165526993 |
| Liver | 26.4 | 31.4 | Kidney (fetal) | 4.8 | 5.3 |
| adenocarcinoma | | | | | |
| Pancreas | 0.4 | 0.9 | Renal ca. 786-0 | 34.2 | 35.8 |
| Pancreatic ca. | 26.6 | 70.2 | Renal ca. A498 | 95.3 | 73.7 |
| CAPAN 2 | | | | | |
| Adrenal gland | 0.3 | 0.3 | Renal ca. RXF 393 | 13.3 | 55.9 |
| Thyroid | 3.7 | 2.7 | Renal ca. | 14.0 | 25.2 |
| | | | ACHN | | |
| Salivary gland | 0.4 | 1.2 | Renal ca. UO-31 | 28.9 | 35.4 |
| Pituitary gland | 1.3 | 1.3 | Renal ca. TK-10 | 19.1 | 17.9 |
| Brain (fetal) | 1.5 | 2.5 | Liver | 1.1 | 1.1 |
| Brain (whole) | 2.6 | 0.0 | Liver (fetal) | 2.7 | 4.2 |
| Brain (amygdala) | 1.8 | 2.0 | Liver ca. | 9.5 | 7.1 |
| | | | (hepatoblast) | | |
| | | | HepG2 | | |
| Brain (cerebellum) | 0.0 | 0.3 | Lung | 2.5 | 1.0 |
| Brain | 26.1 | 4.9 | Lung (fetal) | 3.0 | 1.5 |
| (hippocampus) | | | | | |
| Brain (substantia | 0.1 | 0.2 | Lung ca. | 11.8 | 11.3 |
| nigra) | | | (small cell) | | |
| | | | LX-1 | | |
| Brain (thalamus) | 0.5 | 0.3 | Lung ca. | 13.6 | 3.4 |
| | | | (small cell) | | |
| | | | NCI-H69 | | |
| Cerebral Cortex | 4.1 | 0.8 | Lung ca. (s. cell | 7.4 | 6.4 |
| | | | var.) SHP-77 | | |
| Spinal cord | 0.9 | 2.0 | Lung ca. (large | 2.9 | 15.9 |
| | | | cell) NCI-H460 | | |
| glio/astro U87-MG | 26.6 | 22.2 | Lung ca. (non- | 13.9 | 11.8 |
| | | | sm. cell) A549 | | |
| glio/astro U-118- | 100.0 | 100.0 | Lung ca. (non- | 7.5 | 6.7 |
| MG | | | s. cell) NCI-H23 | | |
| astrocytoma | 53.2 | 62.4 | Lung ca. (non- | 11.3 | 17.0 |
| SW1783 | | | s. cell) HOP-62 | | |
| neuro*; met SK-N- | 0.2 | 0.1 | Lung ca. (non- | 0.2 | 0.1 |
| AS | | | s. cl) NCI-H522 | | |
| astrocytoma SF- | 2.8 | 2.7 | Lung ca. | 45.4 | 92.0 |
| 539 | | | (squam.) SW | | |
| | | | 900 | | |
| astrocytoma SNB- | 69.3 | 71.2 | Lung ca. | 1.6 | 5.3 |
| 75 | | | (squam.) NCI- | | |
| | | | H596 | | |
| glioma SNB-19 | 7.5 | 12.2 | Mammary | 1.5 | 3.2 |
| | | | gland | | |
| glioma U251 | 3.6 | 18.2 | Breast ca.* | 3.9 | 4.2 |
| | | | (pl. ef) MCF-7 | | |
| glioma SF-295 | 27.9 | 25.0 | Breast ca.* | 87.1 | 51.1 |
| | | | (pl. ef) MDA- | | |
| | | | MB-231 | | |
| Heart (fetal) | 0.1 | 0.0 | Breast ca.* | 0.4 | 0.6 |
| | | | (pl. ef) T47D | | |
| Heart | 0.4 | 1.9 | Breast ca. BT-549 | 54.3 | 35.4 |
| Skeletal muscle | 4.6 | 0.5 | Breast ca. | 29.1 | 11.1 |
| (fetal) | | | MDA-N | | |
| Skeletal muscle | 2.0 | 8.9 | Ovary | 0.2 | 0.0 |
| Bone marrow | 0.1 | 0.0 | Ovarian ca. | 1.7 | 4.2 |
| | | | OVCAR-3 | | |
| Thymus | 0.1 | 0.2 | Ovarian ca. | 3.4 | 10.9 |
| | | | OVCAR-4 | | |
| Spleen | 1.3 | 1.0 | Ovarian ca. | 24.1 | 17.1 |
| | | | OVCAR-5 | | |
| Lymph node | 0.4 | 2.4 | Ovarian ca. | 6.8 | 3.2 |
| | | | OVCAR-8 | | |
| Colorectal | 1.6 | 0.9 | Ovarian ca. | 9.2 | 11.3 |
| | | | IGROV-1 | | |
| Stomach | 1.8 | 3.2 | Ovarian ca.* | 12.0 | 11.5 |
| | | | (ascites) SK-OV-3 | | |
| Small intestine | 1.3 | 1.8 | Uterus | 0.8 | 3.0 |
| Colon ca. SW480 | 0.8 | 0.6 | Placenta | 6.8 | 5.8 |
| Colon ca.* | 15.2 | 12.9 | Prostate | 0.5 | 0.5 |
| SW620(SW480 | | | | | |
| met) | | | | | |
| Colon ca. HT29 | 18.9 | 10.1 | Prostate ca.* | 15.2 | 22.5 |
| | | | (bone met) PC-3 | | |
| Colon ca. HCT-116 | 25.2 | 20.7 | Testis | 0.1 | 0.1 |
| Colon ca. CaCo-2 | 15.0 | 11.3 | Melanoma | 3.6 | 3.7 |
| | | | Hs688(A).T | | |
| Colon ca. | 22.4 | 11.0 | Melanoma* | 1.2 | 2.4 |
| tissue(ODO3866) | | | (met) | | |
| | | | Hs688(B).T | | |
| Colon ca. HCC- | 44.4 | 14.4 | Melanoma | 0.3 | 2.5 |
| 2998 | | | UACC-62 | | |
| Gastric ca.* (liver | 52.9 | 52.5 | Melanoma | 0.0 | 0.5 |
| met) NCI-N87 | | | M14 | | |
| Bladder | 2.9 | 3.1 | Melanoma | 5.6 | 2.9 |
| | | | LOX IMVI | | |
| Trachea | 4.8 | 3.7 | Melanoma* | 55.1 | 44.1 |
| | | | (met) SK-MEL-5 | | |
| Kidney | 1.3 | 2.8 | Adipose | 4.1 | 1.9 |

TABLE RE
Panel 2D

| | Rel. Exp.(%) | Rel. Exp.(%) | | Rel. Exp.(%) | Rel. Exp.(%) |
|---|---|---|---|---|---|
| | Ag1684, Run | Ag1684, Run | | Ag1684, Run | Ag1684, Run |
| Tissue Name | 158403763 | 164981024 | Tissue Name | 158403763 | 164981024 |
| Normal Colon | 6.3 | 9.7 | Kidney Margin | 1.0 | 1.0 |
| | | | 8120608 | | |
| CC Well to Mod | 8.8 | 12.6 | Kidney Cancer | 11.3 | 10.2 |
| Diff (ODO3866) | | | 8120613 | | |
| CC Margin | 1.5 | 1.9 | Kidney Margin | 2.7 | 1.7 |
| (ODO3866) | | | 8120614 | | |
| CC Gr.2 | 0.7 | 0.8 | Kidney Cancer | 15.1 | 17.2 |
| rectosigmoid | | | 9010320 | | |
| (ODO3868) | | | | | |
| CC Margin | 0.3 | 0.7 | Kidney Margin | 7.1 | 9.1 |
| (ODO3868) | | | 9010321 | | |
| CC Mod Diff | 4.3 | 6.1 | Normal Uterus | 0.6 | 0.9 |
| (ODO3920) | | | | | |
| CC Margin | 1.1 | 1.0 | Uterus Cancer | 2.5 | 2.5 |
| (ODO3920) | | | 064011 | | |
| CC Gr.2 ascend | 21.8 | 17.3 | Normal | 3.6 | 2.9 |
| colon | | | Thyroid | | |
| (ODO3921) | | | | | |
| CC Margin | 2.2 | 2.1 | Thyroid | 37.1 | 46.0 |
| (ODO3921) | | | Cancer 064010 | | |
| CC from Partial | 32.3 | 22.5 | Thyroid | 31.6 | 26.6 |
| Hepatectomy | | | Cancer | | |
| (ODO4309) Mets | | | A302152 | | |
| Liver Margin | 8.0 | 5.8 | Thyroid | 6.1 | 5.4 |
| (ODO4309) | | | Margin | | |
| | | | A302153 | | |
| Colon mets to | 5.4 | 2.9 | Normal Breast | 4.9 | 3.0 |
| lung (OD04451- | | | | | |
| 01) | | | | | |
| Lung Margin | 2.7 | 3.5 | Breast Cancer | 0.3 | 0.2 |
| (OD04451-02) | | | (OD04566) | | |
| Normal Prostate | 0.5 | 3.0 | Breast Cancer | 1.4 | 1.3 |
| 6546-1 | | | (OD04590-01) | | |
| Prostate Cancer | 1.9 | 1.9 | Breast Cancer | 1.5 | 1.8 |
| (OD04410) | | | Mets | | |
| | | | (OD04590-03) | | |
| Prostate Margin | 1.6 | 1.3 | Breast Cancer | 0.5 | 0.7 |
| (OD04410) | | | Metastasis | | |
| | | | (OD04655-05) | | |
| Prostate Cancer | 3.7 | 2.7 | Breast Cancer | 1.0 | 1.1 |
| (OD04720-01) | | | 064006 | | |
| Prostate Margin | 6.7 | 5.7 | Breast Cancer | 3.3 | 3.0 |
| (OD04720-02) | | | 1024 | | |
| Normal Lung | 7.7 | 6.5 | Breast Cancer | 0.6 | 1.0 |
| 061010 | | | 9100266 | | |
| Lung Met to | 12.9 | 11.3 | Breast Margin | 0.7 | 0.8 |
| Muscle | | | 9100265 | | |
| (ODO4286) | | | | | |
| Muscle Margin | 2.2 | 2.6 | Breast Cancer | 2.0 | 2.5 |
| (ODO4286) | | | A209073 | | |
| Lung Malignant | 8.0 | 7.1 | Breast Margin | 1.8 | 1.6 |
| Cancer | | | A209073 | | |
| (OD03126) | | | | | |
| Lung Margin | 14.8 | 9.7 | Normal Liver | 4.3 | 5.1 |
| (OD03126) | | | | | |
| Lung Cancer | 15.0 | 15.0 | Liver Cancer | 10.9 | 11.0 |
| (OD04404) | | | 064003 | | |
| Lung Margin | 7.8 | 14.7 | Liver Cancer | 3.2 | 2.4 |
| (OD04404) | | | 1025 | | |
| Lung Cancer | 7.6 | 10.7 | Liver Cancer | 2.1 | 2.3 |
| (OD04565) | | | 1026 | | |
| Lung Margin | 3.7 | 5.5 | Liver Cancer | 4.2 | 5.0 |
| (OD04565) | | | 6004-T | | |
| Lung Cancer | 2.6 | 2.0 | Liver Tissue | 4.2 | 5.3 |
| (OD04237-01) | | | 6004-N | | |
| Lung Margin | 4.6 | 4.7 | Liver Cancer | 2.2 | 2.6 |
| (OD04237-02) | | | 6005-T | | |
| Ocular Mel Met | 24.3 | 21.0 | Liver Tissue | 1.1 | 1.2 |
| to Liver | | | 6005-N | | |
| (ODO4310) | | | | | |
| Liver Margin | 8.1 | 7.7 | Normal | 7.7 | 7.6 |
| (ODO4310) | | | Bladder | | |
| Melanoma Mets | 2.6 | 2.4 | Bladder | 0.7 | 0.9 |
| to Lung | | | Cancer 1023 | | |
| (OD04321) | | | | | |
| Lung Margin | 6.7 | 7.1 | Bladder | 4.0 | 4.1 |
| (OD04321) | | | Cancer | | |
| | | | A302173 | | |
| Normal Kidney | 15.5 | 15.2 | Bladder | 36.1 | 46.3 |
| | | | Cancer | | |
| | | | (OD04718-01) | | |
| Kidney Ca, | 100.0 | 100.0 | Bladder | 1.1 | 1.3 |
| Nuclear grade 2 | | | Normal | | |
| (OD04338) | | | Adjacent | | |
| | | | (OD04718-03) | | |
| Kidney Margin | 8.4 | 8.7 | Normal Ovary | 0.1 | 0.0 |
| (OD04338) | | | | | |
| Kidney Ca | 11.6 | 10.4 | Ovarian | 29.3 | 29.3 |
| Nuclear grade 1/2 | | | Cancer 064008 | | |
| (OD04339) | | | | | |
| Kidney Margin | 11.2 | 12.6 | Ovarian | 11.5 | 14.8 |
| (OD04339) | | | Cancer | | |
| | | | (OD04768-07) | | |
| Kidney Ca, Clear | 50.3 | 43.8 | Ovary Margin | 0.9 | 1.2 |
| cell type | | | (OD04768-08) | | |
| (OD04340) | | | | | |
| Kidney Margin | 8.5 | 8.9 | Normal | 3.2 | 3.9 |
| (OD04340) | | | Stomach | | |
| Kidney Ca, | 24.5 | 22.5 | Gastric Cancer | 0.5 | 1.1 |
| Nuclear grade 3 | | | 9060358 | | |
| (OD04348) | | | | | |
| Kidney Margin | 7.1 | 8.4 | Stomach | 2.2 | 3.3 |
| (OD04348) | | | Margin | | |
| | | | 9060359 | | |
| Kidney Cancer | 9.5 | 12.0 | Gastric Cancer | 8.9 | 8.6 |
| (OD04622-01) | | | 9060395 | | |
| Kidney Margin | 1.0 | 1.6 | Stomach | 3.8 | 2.6 |
| (OD04622-03) | | | Margin | | |
| | | | 9060394 | | |
| Kidney Cancer | 24.0 | 23.0 | Gastric Cancer | 17.0 | 33.7 |
| (OD04450-01) | | | 9060397 | | |
| Kidney Margin | 6.9 | 5.8 | Stomach | 1.2 | 1.4 |
| (OD04450-03) | | | Margin | | |
| | | | 9060396 | | |
| Kidney Cancer | 6.0 | 6.5 | Gastric Cancer | 8.8 | 6.8 |
| 8120607 | | | 064005 | | |

TABLE RF
Panel 4D

| | Rel. Exp.(%) | | Rel. Exp.(%) |
|---|---|---|---|
| | Ag1684, Run | | Ag1684, Run |
| Tissue Name | 158404018 | Tissue Name | 158404018 |
| Secondary Th1 act | 0.5 | HUVEC IL-1 beta | 9.3 |
| Secondary Th2 act | 0.1 | HUVEC IFN gamma | 22.5 |
| Secondary Tr1 act | 0.4 | HUVEC TNF alpha + IFN | 17.7 |
| | | gamma | |
| Secondary Th1 rest | 0.0 | HUVEC TNF alpha + IL4 | 9.7 |
| Secondary Th2 rest | 0.0 | HUVEC IL-11 | 5.9 |
| Secondary Tr1 rest | 0.0 | Lung Microvascular EC | 7.2 |
| | | none | |
| Primary Th1 act | 0.1 | Lung Microvascular EC | 5.2 |
| | | TNF alpha + IL-1 beta | |
| Primary Th2 act | 0.5 | Microvascular Dermal EC | 20.2 |
| | | none | |
| Primary Tr1 act | 0.2 | Microsvasular Dermal EC | 8.8 |
| | | TNF alpha + IL-1 beta | |
| Primary Th1 rest | 0.0 | Bronchial epithelium | 11.8 |
| | | TNF alpha + IL-1 beta | |
| Primary Th2 rest | 0.0 | Small airway epithelium | 10.6 |
| | | none | |
| Primary Tr1 rest | 0.0 | Small airway epithelium | 97.3 |
| | | TNF alpha + IL-1 beta | |
| CD45RA CD4 | 7.7 | Coronery artery SMC rest | 4.2 |
| lymphocyte act | | | |
| CD45RO CD4 | 0.2 | Coronery artery SMC | 2.6 |
| lymphocyte act | | TNF alpha + IL-1 beta | |
| CD8 lymphocyte act | 0.0 | Astrocytes rest | 12.3 |
| Secondary CD8 | 0.1 | Astrocytes TNF alpha + IL- | 11.2 |
| lymphocyte rest | | 1 beta | |
| Secondary CD8 | 0.3 | KU-812 (Basophil) rest | 0.1 |
| lymphocyte act | | | |
| CD4 lymphocyte none | 0.0 | KU-812 (Basophil) | 0.8 |
| | | PMA/ionomycin | |
| 2ry Th1/Th2/Tr1_anti- | 0.0 | CCD1106 (Keratinocytes) | 17.3 |
| CD95 CH11 | | none | |
| LAK cells rest | 0.1 | CCD1106 (Keratinocytes) | 7.5 |
| | | TNF alpha + IL-1 beta | |
| LAK cells IL-2 | 0.2 | Liver cirrhosis | 1.4 |
| LAK cells IL-2 + IL-12 | 0.1 | Lupus kidney | 1.5 |
| LAK cells IL-2 + IFN | 0.4 | NCI-H292 none | 62.9 |
| gamma | | | |
| LAK cells IL-2 + IL-18 | 0.3 | NCI-H292 IL-4 | 100.0 |
| LAK cells | 0.9 | NCI-H292 IL-9 | 40.9 |
| PMA/ionomycin | | | |
| NK Cells IL-2 rest | 0.0 | NCI-H292 IL-13 | 46.0 |
| Two Way MLR 3 day | 0.1 | NCI-H292 IFN gamma | 49.3 |
| Two Way MLR 5 day | 0.1 | HPAEC none | 8.0 |
| Two Way MLR 7 day | 0.1 | HPAEC TNF alpha + IL-1 | 11.8 |
| | | beta | |
| PBMC rest | 0.0 | Lung fibroblast none | 4.5 |
| PBMC PWM | 0.7 | Lung fibroblast TNF | 1.7 |
| | | alpha + IL-1 beta | |
| PBMC PHA-L | 0.5 | Lung fibroblast IL-4 | 13.0 |
| Ramos (B cell) none | 0.0 | Lung fibroblast IL-9 | 7.7 |
| Ramos (B cell) | 0.0 | Lung fibroblast IL-13 | 5.7 |
| ionomycin | | | |
| B lymphocytes PWM | 3.1 | Lung fibroblast IFN | 14.8 |
| | | gamma | |
| B lymphocytes CD40L | 1.1 | Dermal fibroblast | 26.4 |
| and IL-4 | | CCD1070 rest | |
| EOL-1 dbcAMP | 0.0 | Dermal fibroblast | 16.7 |
| | | CCD1070 TNF alpha | |
| EOL-1 dbcAMP | 0.0 | Dermal fibroblast | 5.7 |
| PMA/ionomycin | | CCD1070 IL-1 beta | |
| Dendritic cells none | 0.1 | Dermal fibroblast IFN | 3.6 |
| | | gamma | |
| Dendritic cells LPS | 0.2 | Dermal fibroblast IL-4 | 1.9 |
| Dendritic cells anti- | 0.0 | IBD Colitis 2 | 0.2 |
| CD40 | | | |
| Monocytes rest | 0.0 | IBD Crohn's | 0.0 |
| Monocytes LPS | 22.5 | Colon | 4.4 |
| Macrophages rest | 0.3 | Lung | 5.7 |
| Macrophages LPS | 1.2 | Thymus | 12.5 |
| HUVEC none | 11.7 | Kidney | 2.6 |
| HUVEC starved | 18.7 | | |

TABLE RG
Panel 5 Islet

| | Rel. Exp.(%) | | Rel. Exp.(%) |
|---|---|---|---|
| | Ag1684, Run | | Ag1684, Run |
| Tissue Name | 224084580 | Tissue Name | 224084580 |
| 97457_Patient- | 12.7 | 94709_Donor 2 AM - A_adipose | 3.1 |
| 02go_adipose | | | |
| 97476_Patient- | 2.8 | 94710_Donor 2 AM - B_adipose | 15.4 |
| 07sk_skeletal muscle | | | |
| 97477_Patient- | 1.2 | 94711_Donor 2 AM - C_adipose | 9.0 |
| 07ut_uterus | | | |
| 97478_Patient- | 15.4 | 94712_Donor 2 AD - A_adipose | 14.7 |
| 07pl_placenta | | | |
| 99167_Bayer | 30.6 | 94713_Donor 2 AD - B_adipose | 9.5 |
| Patient 1 | | | |
| 97482_Patient- | 2.1 | 94714_Donor 2 AD - C_adipose | 17.0 |
| 08ut_uterus | | | |
| 97483_Patient- | 13.5 | 94742_Donor 3 U - | 1.5 |
| 08pl_placenta | | A_Mesenchymal Stem Cells | |
| 97486_Patient- | 1.6 | 94743_Donor 3 U - | 38.4 |
| 09sk_skeletal muscle | | B_Mesenchymal Stem Cells | |
| 97487_Patient- | 2.8 | 94730_Donor 3 AM - A_adipose | 37.6 |
| 09ut_uterus | | | |
| 97488_Patient- | 7.3 | 94731_Donor 3 AM - B_adipose | 9.9 |
| 09pl_placenta | | | |
| 97492_Patient- | 9.2 | 94732_Donor 3 AM - C_adipose | 4.0 |
| 10ut_uterus | | | |
| 97493_Patient- | 22.5 | 94733_Donor 3 AD - A_adipose | 17.9 |
| 10pl_placenta | | | |
| 97495_Patient- | 15.5 | 94734_Donor 3 AD - B_adipose | 4.7 |
| 11go_adipose | | | |
| 97496_Patient- | 6.8 | 94735_Donor 3 AD - C_adipose | 39.0 |
| 11sk_skeletal muscle | | | |
| 97497_Patient- | 3.0 | 77138_Liver_HepG2untreated | 27.4 |
| 11ut_uterus | | | |
| 97498_Patient- | 13.2 | 73556_Heart_Cardiac stromal cells | 2.5 |
| 11pl_placenta | | (primary) | |
| 97500_Patient- | 22.5 | 81735_Small Intestine | 4.6 |
| 12go_adipose | | | |
| 97501_Patient- | 12.2 | 72409_Kidney_Proximal | 16.5 |
| 12sk_skeletal muscle | | Convoluted Tubule | |
| 97502_Patient- | 3.3 | 82685_Small intestine_Duodenum | 3.5 |
| 12ut_uterus | | | |
| 97503_Patient- | 16.0 | 90650_Adrenal_Adrenocortical | 0.0 |
| 12pl_placenta | | adenoma | |
| 94721_Donor 2 U - | 11.1 | 72410_Kidney_HRCE | 100.0 |
| A_Mesenchymal | | | |
| Stem Cells | | | |
| 94722_Donor 2 U - | 6.9 | 72411_Kidney_HRE | 50.7 |
| B_Mesenchymal Stem | | | |
| Cells | | | |
| 94723_Donor 2 U - | 2.2 | 73139_Uterus_Uterine smooth | 11.7 |
| C_Mesenchymal Stem | | muscle cells | |
| Cells | | | |

CNS_neurodegeneration_v1.0 Summary: Ag1684 This panel does not show differential expression of the CG59885-01 gene in Alzheimer's disease. However, this expression profile confirms the presence of this gene in the brain. Please see Panel 1.4 for discussion of utility of this gene in the central nervous system.

General_screening_panel_v1.4 Summary: Ag1684 Highest expression of the CG59885-01 gene is seen in a renal cancer cell line (CT=20). Overall, expression appears to be much higher in cancer cell lines than in samples from normal tissue. Significant levels of expression are also seen in cell lines derived from pancreatic, brain, colon, gastric, lung, breast, ovarian, and melanoma cancers. Thus, expression of this gene could be used to differentiate between these samples and other samples on this panel and as a marker to detect the presence of cancer. Furthermore, therapeutic modulation of the expression or function of this gene with a human monoclonal antibody is anticipated to limit or block the extent of metastasis and growth in most tumors.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

In addition, this gene is expressed at high levels in all regions of the central nervous system examined, including amygdala, hippocampus, substantia nigra, thalamus, cerebellum, cerebral cortex, and spinal cord. Therefore, this gene may play a role in central nervous system and may be a target of neurologic diseases such as Alzheimer's disease, Parkinson's disease, epilepsy, multiple sclerosis, schizophrenia and depression.

Panel 1.3D Summary: Ag1684 Expression of the CG59885-01 gene confirms the results of Panel 1.4. Please see Panel 1.4 for discussion of utility of this gene in cancer, metabolic disease and the central nervous system.

Panel 2D Summary: Ag1684 Two experiments with the same probe and primer produce results that are in excellent agreement, with highest expression of the CG59885-01 gene in kidney cancer (CTs=24-25). In addition, expression of this gene is higher in gastric, bladder, ovarian, thyroid, kidney and colon cancers when compared to expression in the corresponding normal adjacent tissue. Thus, expression of this gene could be used to differentiate between these samples and other samples on this panel and as a marker to detect the presence of cancer. Furthermore, therapeutic modulation of the expression or function of this gene may be effective in the treatment of these cancers.

Panel 3D Summary: Ag1684 Results from one experiment with the CG59885-01 gene are not included. The amp plot indicates that there were experimental difficulties with this run.

Panel 4D Summary: Ag1684 Highest expression of the CG59885-01 gene is seen in the NCI-H292 mucoepidermoid cell line treated with IL-4 (CT=25). Significant levels of expression are also seen in a cluster of treated and untreated NCI-H292 mucoepidermoid cells, IL-4, IL-9, IL-13 and IFN gamma activated lung fibroblasts, human pulmonary aortic endothelial cells (treated and untreated), small airway epithelium (treated and untreated), treated bronchial epithelium and lung microvascular endothelial cells (treated and untreated). The expression of this gene in cells derived from or within the lung suggests that this gene may be involved in normal conditions as well as pathological and inflammatory lung disorders that include chronic obstructive pulmonary disease, asthma, allergy and emphysema.

Moderate levels of expression are also detected in treated and untreated HUVECs (endothelial cells), coronary artery smooth muscle cells (treated and untreated), treated and untreated astrocytes, KU-812 basophils, keratinocytes, dermal fibroblasts, and normal tissues from lung, colon, thymus and kidney. Expression in these immune cell types and tissues suggests that therapeutic modulation of this gene product may ameliorate symptoms associated with infectious conditions as well as inflammatory and autoimmune disorders that include psoriasis, allergy, asthma, inflammatory bowel disease, rheumatoid arthritis and osteoarthritis.

Interestingly, expression of this gene is stimulated in TNFalpha+IL-1 beta treated small airway epithelium (CT=25) and LPS-treated monocytes (CT=27) as compared to their untreated counterparts (CTs=28-40). Thus, expression of this gene can be used to distinguish these treated cells from their untreated counterparts. This gene codes for a variant of hepatocyte growth factor receptor, HGFR-MET. HGFR-MET is a transmembrane tyrosine kinase proto-oncogene, required for the action of hepatocyte growth factor (HGF) (Ref. 1). Recently, it was shown that HGF modulates the function of monocytes in a paracrine/autocrine manner (Ref.2). The expression of the HGFR-MET gene in LPS-treated monocytes, suggests a role for this gene product in initiating inflammatory reactions. Therefore, modulation of the expression or activity of HGFR-MET through the application of monoclonal antibodies may reduce or prevent early stages of inflammation and reduce the severity of inflammatory diseases such as psoriasis, asthma, inflammatory bowel disease, rheumatoid arthritis, osteoarthritis and other lung inflammatory diseases.

References.

1. Stella M C, Comoglio P M. (1999) HGF: a multifunctional growth factor controlling cell scattering. Int J Biochem Cell Biol December 1999;31(12):1357-62

2. Galimi F, Cottone E, Vigna E, Arena N, Boccaccio C, Giordano S, Naldini L, Comoglio P M. (2001) Hepatocyte growth factor is a regulator of monocyte-macrophage function. J Immunol 166(2):1241-7.

Panel 5 Islet Summary: Ag1684 Highest expression of the CG59885-01 gene is seen in kidney-HRCE samples (CT=29.8). In addition, moderate to low expression is also detected in pancreatic islet cells (patient 1), adipose, mesenchymal stem cells, placenta, skeletal muscle from number of donor and patients. Please see panel 1.4 for the potential utility of this gene.

### S. NOV26a (CG93443-01: Novel LIV-1 Like Gene)

Expression of gene CG93443-01 was assessed using the primer-probe sets Ag2552 and Ag3855, described in Tables SA and SB. Results of the RTQ-PCR runs are shown in Tables SC, SD, SE, SF, SG, and SH.

TABLE SA
Probe Name Ag2552

| | | | | Start |
|---|---|---|---|---|
| Primers | Sequences | | Length | Position |
| Forward | 5′-cttctgataccctgccctagtc-3′ | (SEQ ID NO:278) | 22 | 2167 |
| Probe | TET-5′-ccccacctttgacttaagatcccaca-3′-TAMRA | (SEQ ID NO:282) | 26 | 2189 |
| Reverse | 5′-ctataggggcttctggtttctg-3′ | (SEQ ID NO:280) | 22 | 0 |

TABLE SB
Probe Name Ag3855

| | | | | Start |
|---|---|---|---|---|
| Primer | Sequences | | Length | Position |
| Forward | 5′-cagctgagtggagcctacac-3′ | (SEQ ID NO:281) | 20 | 1140 |
| Probe | TET-5′-caggaccagctcagccagtcagag-3′-TAMRA | (SEQ ID NO:282) | 24 | 1182 |
| Reverse | 5′-cagggagccgtacagatatct-3′ | (SEQ ID NO:283) | 21 | 1206 |

TABLE SC
AI_comprehensive panel_v1.0

| | Rel. Exp.(%) Ag2552, | | Rel. Exp.(%) Ag2552, |
|---|---|---|---|
| Tissue Name | Run 229393908 | Tissue Name | Run 229393908 |
| 110967 COPD-F | 8.3 | 112427 Match Control | 19.6 |
| | | Psoriasis-F | |
| 110980 COPD-F | 5.6 | 112418 Psoriasis-M | 14.8 |
| 110968 COPD-M | 14.7 | 112723 Match Control | 12.7 |
| | | Psoriasis-M | |
| 110977 COPD-M | 13.6 | 112419 Psoriasis-M | 14.0 |
| 110989 Emphysema-F | 23.3 | 112424 Match Control | 12.1 |
| | | Psoriasis-M | |
| 110992 Emphysema-F | 23.5 | 112420 Psoriasis-M | 29.1 |
| 110993 Emphysema-F | 7.3 | 112425 Match Control | 18.4 |
| | | Psoriasis-M | |
| 110994 EmPhysema-F | 2.5 | 104689 (MF) OA Bone- | 10.2 |
| | | Backus | |
| 110995 Emphysema-F | 40.1 | 104690 (MF) Adj | 4.8 |
| | | “Normal” Bone-Backus | |
| 110996 Emphysema-F | 20.6 | 104691 (MF) OA | 4.5 |
| | | Synovium-Backus | |
| 110997 Asthma-M | 11.5 | 104692 (BA) OA | 25.0 |
| | | Cartilage-Backus | |
| 111001 Asthma-F | 5.1 | 104694 (BA) OA Bone- | 9.3 |
| | | Backus | |
| 111002 Asthma-F | 25.0 | 104695 (BA) Adj | 4.8 |
| | | “Normal” Bone-Backus | |
| 111003 Atopic | 16.7 | 104696 (BA) OA | 5.8 |
| Asthma-F | | Synovium-Backus | |
| 111004 Atopic | 45.4 | 104700 (SS) OA Bone- | 6.7 |
| Asthma-F | | Backus | |
| 111005 Atopic | 22.5 | 104701 (SS) Adj | 6.2 |
| Asthma-F | | “Normal” Bone-Backus | |
| 111006 Atopic | 5.1 | 104702 (SS) OA | 9.9 |
| Asthma-F | | Synovium-Backus | |
| 111417 Allergy-M | 11.0 | 117093 OA Cartilage | 8.4 |
| | | Rep7 | |
| 112347 Allergy-M | 21.0 | 112672 OA Bone5 | 6.3 |
| 112349 Normal | 12.5 | 112673 OA Synovium5 | 3.6 |
| Lung-F | | | |
| 112357 Normal | 31.2 | 112674 OA Synovial | 3.0 |
| Lung-F | | Fluid cells5 | |
| 112354 Normal | 10.4 | 117100 OA Cartilage | 9.9 |
| Lung-M | | Rep14 | |
| 112374 Crohns-F | 19.9 | 112756 OA Bone9 | 100.0 |
| 112389 Match Control | 5.6 | 112757 OA Synovium9 | 3.0 |
| Crohns-F | | | |
| 112375 Crohns-F | 13.5 | 112758 OA Synovial | 4.1 |
| | | Fluid Cells9 | |
| 112732 Match Control | 10.4 | 117125 RA Cartilage | 6.1 |
| Crohns-F | | Rep2 | |
| 112725 Crohns-M | 9.3 | 113492 Bone2 RA | 6.8 |
| 112387 Match Control | 6.6 | 113493 Synovium2 RA | 4.0 |
| Crohns-M | | | |
| 112378 Crohns-M | 12.5 | 113494 Syn Fluid Cells | 7.3 |
| | | RA | |
| 112390 Match Control | 13.4 | 113499 Cartilage4 RA | 14.9 |
| Crohns-M | | | |
| 112726 Crohns-M | 15.6 | 113500 Bone4 RA | 16.8 |
| 112731 Match Control | 6.2 | 113501 Synovium4 RA | 13.2 |
| Crohns-M | | | |
| 112380 Ulcer Col-F | 7.0 | 113502 Syn Fluid | 12.2 |
| | | Cells4 RA | |
| 112734 Match Control | 23.5 | 113495 Cartilage3 RA | 7.7 |
| Ulcer Col-F | | | |
| 112384 Ulcer Col-F | 22.7 | 113496 Bone3 RA | 10.1 |
| 112737 Match Control | 17.3 | 113497 Synovium3 RA | 4.7 |
| Ulcer Col-F | | | |
| 112386 Ulcer Col-F | 0.5 | 113498 Syn Fluid | 8.6 |
| | | Cells3 RA | |
| 112738 Match Control | 17.6 | 117106 Normal | 5.7 |
| Ulcer Col-F | | Cartilage Rep20 | |
| 112381 Ulcer Col-M | 2.4 | 113663 Bone3 Normal | 2.6 |
| 112735 Match Control | 12.9 | 113664 Synovium3 | 4.3 |
| Ulcer Col-M | | Normal | |
| 112382 Ulcer Col-M | 7.2 | 113665 Syn Fluid | 4.0 |
| | | Cells3 Normal | |
| 112394 Match Control | 3.7 | 117107 Normal | 1.1 |
| Ulcer Col-M | | Cartilage Rep22 | |
| 112383 Ulcer Col-M | 11.7 | 113667 Bone4 Normal | 3.1 |
| 112736 Match Control | 6.8 | 113668 Synovium4 | 7.2 |
| Ulcer Col-M | | Normal | |
| 112423 Psoriasis-F | 8.1 | 113669 Syn Fluid | 6.8 |
| | | Cells4 Normal | |

TABLE SD
Panel 1.3D

| | Rel. Exp.(%) | Rel. Exp.(%) | | Rel. Exp.(%) | Rel. Exp.(%) |
|---|---|---|---|---|---|
| | Ag2552, Run | Ag2552, Run | | Ag2552, Run | Ag2552, Run |
| Tissue Name | 161905842 | 163728051 | Tissue Name | 161905842 | 163728051 |
| Liver | 0.8 | 0.7 | Kidney (fetal) | 16.3 | 17.0 |
| adenocarcinoma | | | | | |
| Pancreas | 0.7 | 1.4 | Renal ca. 786-0 | 6.9 | 4.7 |
| Pancreatic ca. | 26.4 | 18.6 | Renal ca. A498 | 14.0 | 15.7 |
| CAPAN 2 | | | | | |
| Adrenal gland | 1.5 | 1.1 | Renal ca. RXF | 12.2 | 16.8 |
| | | | 393 | | |
| Thyroid | 8.5 | 15.7 | Renal ca. | 5.0 | 4.0 |
| | | | ACHN | | |
| Salivary gland | 1.7 | 0.8 | Renal ca. UO- | 10.6 | 5.1 |
| | | | 31 | | |
| Pituitary gland | 0.8 | 0.2 | Renal ca. TK- | 16.4 | 10.4 |
| | | | 10 | | |
| Brain (fetal) | 0.4 | 1.1 | Liver | 0.7 | 0.7 |
| Brain (whole) | 1.0 | 2.0 | Liver (fetal) | 3.2 | 3.4 |
| Brain (amygdala) | 3.1 | 2.5 | Liver ca. | 19.9 | 18.8 |
| | | | (hepatoblast) | | |
| | | | HepG2 | | |
| Brain (cerebellum) | 0.6 | 0.5 | Lung | 3.8 | 4.6 |
| Brain | 5.4 | 3.2 | Lung (fetal) | 3.6 | 5.1 |
| (hippocampus) | | | | | |
| Brain (substantia | 0.7 | 0.7 | Lung ca. | 9.8 | 8.2 |
| nigra) | | | (small cell) | | |
| | | | LX-1 | | |
| Brain (thalamus) | 0.4 | 0.1 | Lung ca. | 1.8 | 1.2 |
| | | | (small cell) | | |
| | | | NCI-H69 | | |
| Cerebral Cortex | 10.6 | 12.1 | Lung ca. (s.cell | 13.7 | 16.3 |
| | | | var.) SHP-77 | | |
| Spinal cord | 2.1 | 3.3 | Lung ca. (large | 2.5 | 3.7 |
| | | | cell) NCI-H460 | | |
| glio/astro U87-MG | 14.8 | 18.0 | Lung ca. (non- | 6.0 | 7.3 |
| | | | sm. cell) A549 | | |
| glio/astro U-118- | 2.0 | 1.2 | Lung ca. (non- | 32.3 | 35.1 |
| MG | | | s.cell) NCI- | | |
| | | | H23 | | |
| astrocytoma | 9.5 | 10.2 | Lung ca. (non- | 8.8 | 10.0 |
| SW1783 | | | s.cell) HOP-62 | | |
| neuro*; met SK-N- | 11.9 | 11.4 | Lung ca. (non- | 7.5 | 6.5 |
| AS | | | s.cl) NCI-H522 | | |
| astrocytoma SF- | 5.8 | 6.9 | Lung ca. | 5.3 | 8.4 |
| 539 | | | (squam.) SW | | |
| | | | 900 | | |
| astrocytoma SNB- | 4.2 | 4.5 | Lung ca. | 6.1 | 4.4 |
| 75 | | | (squam.) NCI- | | |
| | | | H596 | | |
| glioma SNB-19 | 0.2 | 0.1 | Mammary | 5.9 | 2.8 |
| | | | gland | | |
| glioma U251 | 0.5 | 1.2 | Breast ca.* | 23.8 | 29.7 |
| | | | (pl.ef) MCF-7 | | |
| glioma SF-295 | 0.3 | 0.0 | Breast ca.* | 10.4 | 9.3 |
| | | | (pl.ef) MDA- | | |
| | | | MB-231 | | |
| Heart (fetal) | 16.8 | 18.8 | Breast ca.* | 7.8 | 10.2 |
| | | | (pl.ef) T47D | | |
| Heart | 1.6 | 1.9 | Breast ca. BT- | 5.3 | 6.6 |
| | | | 549 | | |
| Skeletal muscle | 24.1 | 28.5 | Breast ca. | 7.7 | 10.0 |
| (fetal) | | | MDA-N | | |
| Skeletal muscle | 4.8 | 4.7 | Ovary | 15.4 | 15.9 |
| Bone marrow | 1.3 | 1.2 | Ovarian ca. | 10.1 | 7.6 |
| | | | OVCAR-3 | | |
| Thymus | 11.4 | 9.5 | Ovarian ca. | 11.0 | 14.6 |
| | | | OVCAR-4 | | |
| Spleen | 3.0 | 3.2 | Ovarian ca. | 4.7 | 2.7 |
| | | | OVCAR-5 | | |
| Lymph node | 0.8 | 0.7 | Ovarian ca. | 10.4 | 10.4 |
| | | | OVCAR-8 | | |
| Colorectal | 16.8 | 16.7 | Ovarian ca. | 35.8 | 31.4 |
| | | | IGROV-1 | | |
| Stomach | 2.7 | 2.6 | Ovarian ca.* | 17.4 | 18.7 |
| | | | (ascites) SK- | | |
| | | | OV-3 | | |
| Small intestine | 20.3 | 15.8 | Uterus | 1.3 | 0.6 |
| Colon ca. SW480 | 11.9 | 10.8 | Placenta | 1.8 | 2.2 |
| Colon ca.* | 8.8 | 6.9 | Prostate | 2.3 | 3.9 |
| SW620(SW480 | | | | | |
| met) | | | | | |
| Colon ca. HT29 | 7.5 | 6.7 | Prostate ca.* | | |
| | | | (bone met)PC-3 | 27.7 | 37.9 |
| Colon ca. HCT- | 16.6 | 17.3 | Testis | 6.5 | 5.2 |
| 116 | | | | | |
| Colon ca. CaCo-2 | 8.9 | 8.1 | Melanoma | 1.9 | 2.5 |
| | | | Hs688(A).T | | |
| Colon ca. | 59.9 | 64.6 | Melanoma* | 1.5 | 2.5 |
| tissue(ODO3866) | | | (met) | | |
| | | | Hs688(B).T | | |
| Colon ca. HCC- | 9.9 | 11.8 | Melanoma | 3.3 | 4.7 |
| 2998 | | | UACC-62 | | |
| Gastric ca.* (liver | 100.0 | 100.0 | Melanoma | 3.1 | 3.3 |
| met) NCI-N87 | | | M14 | | |
| Bladder | 11.1 | 10.0 | Melanoma | 7.2 | 5.1 |
| | | | LOX IMVI | | |
| Trachea | 6.5 | 5.7 | Melanoma* | 7.3 | 6.8 |
| | | | (met) SK- | | |
| | | | MEL-5 | | |
| Kidney | 58.6 | 54.0 | Adipose | 4.0 | 1.5 |

TABLE SE
Panel 2D

| | Rel. Exp.(%) | Rel. Exp.(%) | | Rel. Exp.(%) | Rel. Exp.(%) |
|---|---|---|---|---|---|
| | Ag2552, Run | Ag2552, Run | | Ag2552, Run | Ag2552, Run |
| Tissue Name | 161905843 | 163578013 | Tissue Name | 161905843 | 163578013 |
| Normal Colon | 26.8 | 27.5 | Kidney Margin | 51.1 | 52.5 |
| | | | 8120608 | | |
| CC Well to Mod | 25.9 | 36.6 | Kidney Cancer | 33.0 | 38.4 |
| Diff(ODO3866) | | | 8120613 | | |
| CC Margin | 10.3 | 13.6 | Kidney Margin | 81.8 | 65.1 |
| (ODO3866) | | | 8120614 | | |
| CC Gr.2 | 20.9 | 27.9 | Kidney Cancer | 10.7 | 15.0 |
| rectosigmoid | | | 9010320 | | |
| (ODO3868) | | | | | |
| CC Margin | 1.8 | 1.7 | Kidney Margin | 45.7 | 50.3 |
| (ODO3868) | | | 9010321 | | |
| CC Mod Diff | 42.6 | 52.5 | Normal Uterus | 1.6 | 2.2 |
| (ODO3920) | | | | | |
| CC Margin | 7.5 | 9.0 | Uterus Cancer | 5.8 | 8.2 |
| (ODO3920) | | | 064011 | | |
| CC Gr.2 ascend | 100.0 | 94.6 | Normal | 2.8 | 7.5 |
| colon | | | Thyroid | | |
| (ODO3921) | | | | | |
| CC Margin | 11.7 | 11.3 | Thyroid | 9.5 | 11.7 |
| (ODO3921) | | | Cancer 064010 | | |
| CC from Partial | 81.8 | 95.9 | Thyroid | 3.9 | 6.3 |
| Hepatectomy | | | Cancer | | |
| (ODO4309) Mets | | | A302152 | | |
| Liver Margin | 3.7 | 3.5 | Thyroid | 17.0 | 19.8 |
| (ODO4309) | | | Margin | | |
| | | | A302153 | | |
| Colon mets to | 19.1 | 14.7 | Normal Breast | 13.2 | 5.6 |
| lung (OD04451- | | | | | |
| 01) | | | | | |
| Lung Margin | 5.0 | 5.9 | Breast Cancer | 11.7 | 12.9 |
| (OD04451-02) | | | (OD04566) | | |
| Normal Prostate | 2.0 | 20.2 | Breast Cancer | 11.8 | 13.5 |
| 6546-1 | | | (OD04590-01) | | |
| Prostate Cancer | 7.6 | 10.2 | Breast Cancer | 10.4 | 14.7 |
| (OD04410) | | | Mets | | |
| | | | (OD04590-03) | | |
| Prostate Margin | 12.6 | 12.1 | Breast Cancer | 43.2 | 49.0 |
| (OD04410) | | | Metastasis | | |
| | | | (OD04655-05) | | |
| Prostate Cancer | 6.1 | 6.2 | Breast Cancer | 12.6 | 15.8 |
| (OD04720-01) | | | 064006 | | |
| Prostate Margin | 8.4 | 10.3 | Breast Cancer | 17.0 | 17.0 |
| (OD04720-02) | | | 1024 | | |
| Normal Lung | 11.3 | 11.3 | Breast Cancer | 10.0 | 27.0 |
| 061010 | | | 9100265 | | |
| Lung Met to | 15.0 | 12.9 | Breast Margin | 7.4 | 11.2 |
| Muscle | | | 9100265 | | |
| (ODO4286) | | | | | |
| Muscle Margin | 2.6 | 4.6 | Breast Cancer | 21.5 | 25.3 |
| (ODO4286) | | | A209073 | | |
| Lung Malignant | 21.0 | 23.2 | Breast Margin | 8.2 | 8.8 |
| Cancer | | | A209073 | | |
| (OD03126) | | | | | |
| Lung Margin | 13.4 | 12.9 | Normal Liver | 1.5 | 3.0 |
| (OD03126) | | | | | |
| Lung Cancer | 20.0 | 17.6 | Liver Cancer | 0.4 | 0.1 |
| (OD04404) | | | 064003 | | |
| Lung Margin | 2.6 | 4.5 | Liver Cancer | 1.4 | 1.6 |
| (OD04404) | | | 1025 | | |
| Lung Cancer | 9.5 | 13.1 | Liver Cancer | 10.7 | 15.2 |
| (OD04565) | | | 1026 | | |
| Lung Margin | 4.2 | 5.8 | Liver Cancer | 2.3 | 2.0 |
| (OD04565) | | | 6004-T | | |
| Lung Cancer | 18.8 | 20.0 | Liver Tissue | 22.5 | 22.2 |
| (OD04237-01) | | | 6004-N | | |
| Lung Margin | 6.3 | 6.7 | Liver Cancer | 11.0 | 10.6 |
| (OD04237-02) | | | 6005-T | | |
| Ocular Mel Met | 20.2 | 28.1 | Liver Tissue | 1.4 | 1.9 |
| to Liver | | | 6005-N | | |
| (ODO4310) | | | | | |
| Liver Margin | 2.0 | 2.5 | Normal | 13.0 | 13.4 |
| (ODO4310) | | | Bladder | | |
| Melanoma Mets | 7.3 | 10.5 | Bladder | 8.1 | 6.3 |
| to Lung | | | Cancer 1023 | | |
| (OD04321) | | | | | |
| Lung Margin | 9.3 | 4.8 | Bladder | 4.8 | 5.6 |
| (OD04321) | | | Cancer | | |
| | | | A302173 | | |
| Normal Kidney | 26.6 | 19.9 | Bladder | 52.1 | 50.7 |
| | | | Cancer | | |
| | | | (OD04718-01) | | |
| Kidney Ca, | 2.1 | 2.4 | Bladder | 1.8 | 3.4 |
| Nuclear grade 2 | | | Normal | | |
| (OD04338) | | | Adjacent | | |
| | | | (OD04718-03) | | |
| Kidney Margin | 7.3 | 8.4 | Normal Ovary | 5.3 | 5.8 |
| (OD04338) | | | | | |
| Kidney Ca | 12.2 | 11.3 | Ovarian | 76.8 | 83.5 |
| Nuclear grade 1/2 | | | Cancer 064008 | | |
| (OD04339) | | | | | |
| Kidney Margin | 31.0 | 36.3 | Ovarian | 85.3 | 100.0 |
| (OD04339) | | | Cancer | | |
| | | | (OD04768-07) | | |
| Kidney Ca, Clear | 6.0 | 7.9 | Ovary Margin | 2.3 | 1.2 |
| cell type | | | (OD04768-08) | | |
| (OD04340) | | | | | |
| Kidney Margin | 24.5 | 22.1 | Normal | 4.3 | 2.9 |
| (OD04340) | | | Stomach | | |
| Kidney Ca, | 5.7 | 7.2 | Gastric Cancer | 3.6 | 5.5 |
| Nuclear grade 3 | | | 9060358 | | |
| (OD04348) | | | | | |
| Kidney Margin | 11.8 | 15.0 | Stomach | 9.0 | 8.3 |
| (OD04348) | | | Margin | | |
| | | | 9060359 | | |
| Kidney Cancer | 6.7 | 8.4 | Gastric Cancer | 18.6 | 30.1 |
| (OD04622-01) | | | 9060395 | | |
| Kidney Margin | 5.0 | 4.8 | Stomach | 21.6 | 20.7 |
| (OD04622-03) | | | Margin | | |
| | | | 9060394 | | |
| Kidney Cancer | 5.4 | 12.8 | Gastric Cancer | 84.7 | 81.8 |
| (OD04450-01) | | | 9060397 | | |
| Kidney Margin | 8.7 | 10.7 | Stomach | 9.2 | 10.1 |
| (OD04450-03) | | | Margin | | |
| | | | 9060396 | | |
| Kidney Cancer | 4.8 | 6.0 | Gastric Cancer | 14.5 | 12.9 |
| 8120607 | | | 064005 | | |

TABLE SF
Panel 3D

| | Rel. Exp.(%) | | Rel. Exp.(%) |
|---|---|---|---|
| | Ag2552, Run | | Ag2552, Run |
| Tissue Name | 163482707 | Tissue Name | 163482707 |
| Daoy-Medulloblastoma | 0.6 | Ca Ski-Cervical epidermoid | 47.3 |
| | | carcinoma (metastasis) | |
| TE671-Medulloblastoma | 0.0 | ES-2-Ovarian clear cell | 10.2 |
| | | carcinoma | |
| D283 Med- | 30.8 | Ramos-Stimulated with | 0.0 |
| Medulloblastoma | | PMA/ionomycin 6h | |
| PFSK-1-Primitive | 3.7 | Ramos-Stimulated with | 0.0 |
| Neuroectodermal | | PMA/ionomycin 14h | |
| XF-498-CNS | 1.8 | MEG-01-Chronic | |
| | | myelogenous leukemia | 17.3 |
| | | (megokaryoblast) | |
| SNB-78-Glioma | 0.4 | Raji-Burkitt's lymphoma | 9.3 |
| SF-268-Glioblastoma | 2.6 | Daudi-Burkitt's lymphoma | 0.0 |
| T98G-Glioblastoma | 0.2 | U266-B-cell plasmacytoma | 6.7 |
| SK-N-SH- | 7.6 | CA46-Burkitt's lymphoma | 12.2 |
| Neuroblastoma | | | |
| (metastasis) | | | |
| SF-295-Glioblastoma | 0.0 | RL-non-Hodgkin's B-cell | 6.6 |
| | | lymphoma | |
| Cerebellum | 0.3 | JM1-pre-B-cell lymphoma | 9.9 |
| Cerebellum | 0.5 | Jurkat-T cell leukemia | 9.2 |
| NCI-H292- | 0.0 | TF-1 -Erythroleukemia | 74.7 |
| Mucoepidermoid lung | | | |
| carcinoma | | | |
| DMS-114-Small cell | 4.2 | HUT 78-T-cell lymphoma | 11.3 |
| lung cancer | | | |
| DMS-79-Small cell lung | 54.0 | U937-Histiocytic lymphoma | 22.2 |
| cancer | | | |
| NCI-H146-Small cell | 16.7 | KU-812-Myelogenous | 56.3 |
| lung cancer | | leukemia | |
| NCI-H526-Small cell | 31.0 | 769-P-Clear cell renal | 16.2 |
| lung cancer | | carcinoma | |
| NCI-N417-Small cell | 7.9 | Caki-2-Clear cell renal | 23.2 |
| lung cancer | | carcinoma | |
| NCI-H82-Small cell lung | 10.5 | SW 839-Clear cell renal | 8.0 |
| cancer | | carcinoma | |
| NCI-H157-Squamous | 8.6 | G401-Wilms'tumor | 1.6 |
| cell lung cancer | | | |
| (metastasis) | | | |
| NCI-H1155-Large cell | 25.3 | Hs766T-Pancreatic carcinoma | 3.2 |
| lung cancer | | (LN metastasis) | |
| NCI-H1299-Large cell | 21.8 | CAPAN-1-Pancreatic | 19.1 |
| lung cancer | | adenocarcinoma (liver | |
| | | metastasis) | |
| NCI-H727-Lung | 12.7 | SU86.86-Pancreatic | 11.3 |
| carcinoid | | carcinoma (liver metastasis) | |
| NCI-UMC-11-Lung | 19.2 | BxPC-3-Pancreatic | 18.7 |
| carcinoid | | adenocarcinoma | |
| LX-1-Small cell lung | 10.7 | HPAC-Pancreatic | 33.9 |
| cancer | | adenocarcinoma | |
| Colo-205-Colon cancer | 17.4 | MIA PaCa-2-Pancreatic | 2.0 |
| | | carcinoma | |
| KM12-Colon cancer | 31.0 | CFPAC-1-Pancreatic ductal | 26.6 |
| | | adenocarcinoma | |
| KM20L2-Colon cancer | 15.5 | PANC-1-Pancreatic | 12.9 |
| | | epithelioid ductal carcinoma | |
| NCI-H716-Colon cancer | 17.9 | T24-Bladder carcinma | 7.7 |
| | | (transitional cell) | |
| SW-48-Colon | 44.8 | 5637-Bladder carcinoma | 15.0 |
| adenocarcinoma | | | |
| SW1116-Colon | 4.7 | HT-1197-Bladder carcinoma | 13.2 |
| adenocarcinoma | | | |
| LS 174T-Colon | 20.7 | UM-UC-3-Bladder carcinma | 0.8 |
| adenocarcinoma | | (transitional cell) | |
| SW-948-Colon | 4.1 | A204-Rhabdomyosarcoma | 1.6 |
| adenocarcinoma | | | |
| SW-480-Colon | 16.8 | HT-1080-Fibrosarcoma | 3.4 |
| adenocarcinoma | | | |
| NCI-SNU-5-Gastric | 13.8 | MG-63-Osteosarcoma | 3.9 |
| carcinoma | | | |
| KATO III-Gastric | 100.0 | SK-LMS-1-Leiomyosarcoma | 3.9 |
| carcinoma | | (vulva) | |
| NCI-SNU-16-Gastric | 0.2 | SJRH30-Rhabdomyosarcoma | 0.1 |
| carcinoma | | (met to bone marrow) | |
| NCI-SNU-1-Gastric | 33.7 | A431-Epidermoid carcinoma | 6.9 |
| carcinoma | | | |
| RF-1-Gastric | 0.0 | WM266-4-Melanoma | 5.0 |
| adenocarcinoma | | | |
| RF-48-Gastric | 0.0 | DU 145-Prostate carcinoma | 0.1 |
| adenocarcinoma | | (brain metastasis) | |
| MKN-45-Gastric | 8.4 | MDA-MB-468-Breast | 4.7 |
| carcinoma | | adenocarcinoma | |
| NCI-N87-Gastric | 29.9 | SCC-4-Squamous cell | 0.3 |
| carcinoma | | carcinoma of tongue | |
| OVCAR-5-Ovarian | 3.7 | SCC-9-Squamous cell | 0.4 |
| carcinoma | | carcinoma of tongue | |
| RL95-2-Uterine | 13.6 | SCC-15-Squamous cell | 0.1 |
| carcinoma | | carcinoma of tongue | |
| HelaS3-Cervical | 2.7 | CAL 27-Squamous cell | 15.4 |
| adenocarcinoma | | carcinoma of tongue | |

TABLE SG
Panel 4.1D

| | Rel. Exp.(%) | | Rel. Exp.(%) |
|---|---|---|---|
| | Ag3855, Run | | Ag3855, Run |
| Tissue Name | 170120695 | Tissue Name | 170120695 |
| Secondary Th1 act | 0.0 | HUVEC IL-1 beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1 beta | |
| Primary Th2 act | 0.0 | Microvascular Dermal EC | 0.0 |
| | | none | |
| Primary Tr1 act | 0.0 | Microvascular Dermal EC | 0.0 |
| | | TNF alpha + IL-1 beta | |
| primary Th1 rest | 0.0 | Bronchial epithelium | 0.0 |
| | | TNF alpha + IL 1 beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1 beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1 beta | |
| CD8 lymphocyte act | 0.0 | Astrocytes rest | 0.0 |
| Secondary CD8 | 0.0 | Astrocytes TNF alpha + IL- | 0.0 |
| lymphocyte rest | | 1 beta | |
| Secondary CD8 | 0.0 | KU-812 (Basophil) rest | 0.1 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1 beta | |
| LAK cells IL-2 | 0.0 | Liver cirrhosis | 0.0 |
| LAK cells IL-2 + IL-12 | 0.0 | NCI-H292 none | 0.0 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 0.0 |
| LAK cells | 0.0 | NCI-H292 IL-13 | 0.0 |
| PMA/ionomycin | | | |
| NK Cells IL-2 rest | 0.0 | NCI-H292 IFN gamrna | 0.0 |
| Two Way MLR 3 day | 0.0 | HPAEC none | 0.0 |
| Two Way MLR 5 day | 0.0 | HPAEC TNF alpha + IL-1 | 0.0 |
| | | beta | |
| Two Way MLR 7 day | 0.0 | Lung fibroblast none | 0.0 |
| PBMC rest | 0.0 | Lung fibroblast TNF | 0.0 |
| | | alpha + IL-1 beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) | 100.0 | Lung fibroblast IFN | 0.0 |
| ionomycin | | gamma | |
| B lymphocytes PWM | 0.0 | Dermal fibroblast | 0.0 |
| | | CCD1070 rest | |
| B lymphocytes CD40L | 0.0 | Dermal fibroblast | 0.0 |
| and IL-4 | | CCD1070 TNF alpha | |
| EOL-1 dbcAMP | 0.0 | Dermal fibroblast | 0.0 |
| | | CCD1070 IL-1 beta | |
| EOL-1 dbcAMP | 0.0 | Dermal fibroblast IFN | 0.0 |
| PMA/ionomycin | | gamma | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti- | 0.0 | Neutrophils TNFa + LPS | 0.0 |
| CD40 | | | |
| Monocytes rest | 0.0 | Neutrophils rest | 0.0 |
| Monocytes LPS | 0.0 | Colon | 1.8 |
| Macrophages rest | 0.0 | Lung | 0.0 |
| Macrophages LPS | 0.0 | Thymus | 0.0 |
| HUVEC none | 0.0 | Kidney | 1.5 |
| HUVEC starved | 0.0 | | |

TABLE SH
Panel 4D

| | Rel. | Rel. | Rel. | | Rel. | Rel. | Rel. |
|---|---|---|---|---|---|---|---|
| | Exp.(%) | Exp.(%) | Exp.(%) | | Exp.(%) | Exp.(%) | Exp.(%) |
| | Ag2552, | Ag2552, | Ag2552, | | Ag2552, | Ag2552, | Ag2552, |
| | Run | Run | Run | | Run | Run | Run |
| Tissue Name | 161905844 | 163724375 | 163727960 | Tissue Name | 161905844 | 163724375 | 163727960 |
| Secondary Th1 act | 5.9 | 9.7 | 9.9 | HUVEC IL- | 1.0 | 1.2 | 0.8 |
| | | | | 1 beta | | | |
| Secondary Th2 act | 8.8 | 7.9 | 9.7 | HUVEC IFN | 5.6 | 2.9 | 3.1 |
| | | | | gamma | | | |
| Secondary Tr1 act | 10.6 | 8.6 | 11.7 | HUVEC TNF | 2.9 | 3.5 | 4.0 |
| | | | | alpha + IFN | | | |
| | | | | gamma | | | |
| Secondary Th1 | 1.1 | 0.5 | 1.7 | HUVEC TNF | 3.6 | 5.0 | 4.2 |
| rest | | | | alpha + IL4 | | | |
| Secondary Th2 | 2.1 | 1.5 | 1.7 | HUVEC IL-11 | 2.3 | 3.4 | 3.0 |
| rest | | | | | | | |
| Secondary Tr1 | 1.0 | 1.3 | 1.5 | Lung | 4.3 | 6.7 | 6.6 |
| rest | | | | Microvascular | | | |
| | | | | EC none | | | |
| Primary Th1 act | 7.0 | 5.7 | 5.7 | Lung | 2.2 | 4.9 | 5.8 |
| | | | | Microvascular | | | |
| | | | | EC TNF alpha + | | | |
| | | | | IL-1 beta | | | |
| Primary Th2 act | 4.8 | 6.5 | 4.4 | Microvascular | 5.1 | 5.7 | 5.4 |
| | | | | Dermal EC none | | | |
| Primary Tr1 act | 8.7 | 6.6 | 7.4 | Microsvascular | 3.4 | 3.0 | 2.7 |
| | | | | Dermal EC | | | |
| | | | | TNF alpha + IL- | | | |
| | | | | 1 beta | | | |
| Primary Th1 rest | 8.7 | 9.9 | 12.7 | Bronchial | 2.6 | 14.6 | 14.6 |
| | | | | epithelium | | | |
| | | | | TNF alpha + | | | |
| | | | | IL1 beta | | | |
| Primary Th2 rest | 6.7 | 4.8 | 4.5 | Small airway | 7.1 | 6.9 | 7.3 |
| | | | | epithelium none | | | |
| Primary Tr1 rest | 12.4 | 6.7 | 10.8 | Small airway | 26.8 | 23.3 | 27.9 |
| | | | | epithelium | | | |
| | | | | TNF alpha + IL- | | | |
| | | | | 1 beta | | | |
| CD45RA CD4 | 4.3 | 5.7 | 6.6 | Coronery artery | 1.6 | 1.7 | 2.3 |
| lymphocyte act | | | | SMC rest | | | |
| CD45RO CD4 | 8.1 | 8.5 | 9.9 | Coronery artery | 1.1 | 1.6 | 1.3 |
| lymphocyte act | | | | SMC TNF alpha + | | | |
| | | | | IL-1 beta | | | |
| CD8 lymphocyte | 9.9 | 7.6 | 12.1 | Astrocytes rest | 6.1 | 5.6 | 5.8 |
| act | | | | | | | |
| Secondary CD8 | 15.9 | 11.7 | 14.9 | Astrocytes | 3.2 | 3.7 | 5.3 |
| lymphocyte rest | | | | TNF alpha + IL- | | | |
| | | | | 1 beta | | | |
| Secondary CD8 | 2.6 | 4.1 | 3.5 | KU-812 | 81.2 | 78.5 | 90.1 |
| lymphocyte act | | | | (Basophil) rest | | | |
| CD4 lymphocyte | 1.7 | 1.6 | 1.5 | KU-812 | 100.0 | 100.0 | 100.0 |
| none | | | | (Basophil) | | | |
| | | | | PMA/ionomycin | | | |
| 2ry | 2.9 | 3.8 | 5.1 | CCD1106 | 15.4 | 18.6 | 21.9 |
| Th1/Th2/Tr1_anti- | | | | (Keratinocytes) | | | |
| CD95 CH11 | | | | none | | | |
| LAK cells rest | 4.1 | 3.5 | 4.0 | CCD1106 | 2.6 | 10.6 | 15.1 |
| | | | | (Keratinocytes) | | | |
| | | | | TNF alpha + IL- | | | |
| | | | | 1 beta | | | |
| LAK cells IL-2 | 6.8 | 6.0 | 8.5 | Liver cirrhosis | 1.2 | 1.2 | 1.6 |
| LAK cells IL- | 6.2 | 7.5 | 5.8 | Lupus kidney | 1.7 | 1.6 | 2.7 |
| 2 + IL-12 | | | | | | | |
| LAK cells IL- | 10.2 | 6.6 | 7.5 | NCI-H292 none | 0.1 | 0.0 | 0.0 |
| 2 + IFN gamma | | | | | | | |
| LAK cells IL-2 + | 13.5 | 8.0 | 6.8 | NCI-H292 IL-4 | 0.1 | 0.1 | 0.1 |
| IL-18 | | | | | | | |
| LAK cells | 2.5 | 2.8 | 3.1 | NCI-H292 IL-9 | 0.0 | 0.1 | 0.2 |
| PMA/ionomycin | | | | | | | |
| NK Cells IL-2 rest | 3.2 | 3.4 | 3.3 | NCI-H292 IL- | 0.1 | 0.2 | 0.0 |
| | | | | 13 | | | |
| Two Way MLR 3 | 5.2 | 4.9 | 6.1 | NCI-H292 IFN | 0.1 | 0.0 | 0.2 |
| day | | | | gamma | | | |
| Two Way MLR 5 | 4.2 | 4.7 | 6.0 | HPAEC none | 3.1 | 4.8 | 6.1 |
| day | | | | | | | |
| Two Way MLR 7 | 4.3 | 5.6 | 5.6 | HPAEC TNF | 2.7 | 3.5 | 3.0 |
| day | | | | alpha + IL-1 | | | |
| | | | | beta | | | |
| PBMC rest | 2.3 | 2.4 | 2.5 | Lung fibroblast | 7.6 | 6.7 | 7.6 |
| | | | | none | | | |
| PBMC PWM | 14.4 | 16.7 | 18.7 | Lung fibroblast | 4.1 | 3.8 | 4.2 |
| | | | | TNF alpha + IL- | | | |
| | | | | 1 beta | | | |
| PBMC PHA-L | 10.2 | 10.5 | 12.3 | Lung fibroblast | 12.0 | 10.7 | 14.2 |
| | | | | IL-4 | | | |
| Ramos (B cell) | 0.0 | 0.0 | 0.0 | Lung fibroblast | 11.1 | 10.9 | 11.4 |
| none | | | | IL-9 | | | |
| Ramos (B cell) | 0.0 | 0.0 | 0.0 | Lung fibroblast | 8.9 | 9.8 | 9.2 |
| ionomycin | | | | IL-13 | | | |
| B lymphocytes | 73.2 | 42.0 | 48.6 | Lung fibroblast | 9.2 | 12.1 | 17.0 |
| PWM | | | | IFN gamma | | | |
| B lymphocytes | 31.9 | 13.4 | 15.5 | Dermal | 8.7 | 8.8 | 11.2 |
| CD40L and IL-4 | | | | fibroblast | | | |
| | | | | CCD1070 rest | | | |
| EOL-1 dbcAMP | 29.5 | 26.1 | 26.4 | Dermal | 15.4 | 11.0 | 13.6 |
| | | | | fibroblast | | | |
| | | | | CCD1070 TNF | | | |
| | | | | alpha | | | |
| EOL-1 dbcAMP | 14.0 | 13.1 | 11.4 | Dermal | 6.0 | 5.9 | 4.4 |
| PMA/ionomycin | | | | fibroblast | | | |
| | | | | CCD1070 IL-1 | | | |
| | | | | beta | | | |
| Dendritic cells | 5.1 | 4.2 | 3.9 | Dermal | 1.7 | 2.7 | 3.1 |
| none | | | | fibroblast IFN | | | |
| | | | | gamma | | | |
| Dendritic cells | 3.5 | 1.9 | 3.3 | Dermal | 3.3 | 3.3 | 3.9 |
| LPS | | | | fibroblast IL-4 | | | |
| Dendritic cells | 5.5 | 7.2 | 7.4 | IBD Colitis 2 | 0.5 | 0.3 | 0.4 |
| anti-CD40 | | | | | | | |
| Monocytes rest | 5.7 | 5.3 | 6.8 | IBD Crohn's | 6.4 | 6.6 | 10.9 |
| Monocytes LPS | 1.0 | 0.9 | 1.6 | Colon | 68.8 | 67.4 | 68.8 |
| Macrophages rest | 10.9 | 8.7 | 9.9 | Lung | 8.8 | 8.1 | 7.6 |
| Macrophages LPS | 1.9 | 1.3 | 2.8 | Thymus | 53.6 | 51.4 | 59.9 |
| HUVEC none | 4.3 | 3.1 | 3.2 | Kidney | 7.9 | 8.2 | 7.8 |
| HUVEC starved | 20.3 | 4.4 | 3.8 | | | | |

AI_comprehensive panel_v1.0 Summary: Ag2552 Highest expression of the CG93443-01 gene is detected in 112756 osteoarthritis bone9 sample (CT=28). Moderate expression of this gene is seen most of the samples used in this panel. Please see panel 4D for the potential utility of this gene. Results from a second experiment with this gene suggests that expression of this gene is low/undetectable (CTs>35) across all of the samples on this panel.

CNS_neurodegeneration_v1.0 Summary: Ag3855 Expression of the CG93443-01 gene is low/undetectable (CTs>35) across all of the samples on this panel.

General_screening_panel_v1.4 Summary: Ag3855 Results from one experiment with the CG93443-01 gene are not included. The amp plot indicates that there were experimental difficulties with this run.

Panel 1.3D Summary: Ag2552 Two experiments with the same probe and primer produce results that are in excellent agreement, with highest expression of the CG93443-01 gene in gastric cancer NCI-N87 cell line (CTs=27-28). In addition, significant expression of this gene is seen in renal, ovarian, breast, lung, liver, pancreatic and colon cancer cell lines, as well as in melanomas. Thus, therapeutic modulation of the expression or function of this gene may be effective in the treatment of these cancers.

Among tissues with metabolic or endocrine function, this gene is expressed at low to moderate levels in pancrease, adipose, adrenal gland, thyroid, skeletal muscle, heart, liver and the gastrointestinal tract. Therefore, therapeutic modulation of the activity of this gene may prove useful in the treatment of endocrine/metabolically related diseases, such as obesity and diabetes.

Interestingly, this gene is expressed at much higher levels in fetal (CTs=30) when compared to adult heart (CTs=33.4-34). This observation suggests that expression of this gene can be used to distinguish fetal from adult heart.

In addition, this gene is expressed at moderate levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 2D Summary: Ag2552 Two experiments with the same probe and primer produce results that are in excellent agreement, with high expression of the CG93443-01 gene in colon cancer grade 2 ascend colon (ODO3921), colon cancer from Partial hepatectomy (ODO4309) metastasis, and ovarian cancer (CTs=27). In addition, expression of this gene is higher in gastric, bladder, ovarian, and colon cancers when compared to expression in the corresponding normal adjacent tissue. Thus, expression of this gene could be used to differentiate between these samples and other samples on this panel and as a marker to detect the presence of cancer. Furthermore, therapeutic modulation of the expression or function of this gene may be effective in the treatment of these cancers.

Panel 3D Summary: Ag2552 Highest expression of the CG93443-01 gene is detected in KATO III-gastric carcinoma (CT=25.5). In addition, expression of this gene is higher in medulloblastoma, small cell lung cancer, gastric, cervical epidermoid carcinoma, B and T cell leukemia and lymphomas, erythroleukemia, KU-812 myelogenous leukemia, pancreatic, bladder and colon cancers. Thus, expression of this gene could be used to differentiate between these samples and other samples on this panel and as a marker to detect the presence of cancer. Furthermore, therapeutic modulation of the expression or function of this gene may be effective in the treatment of these cancers.

Panel 4.1D Summary: Ag3855 Highest expression of the CG93443-01 gene is detected exclusively in ionomycin treated Ramos B cells (CT=26.8). Furthermore, expression of this gene is low/undetectable in resting Ramos B cells (CT=40). Thus, expression of this gene can be used to distinguish ionomycin treated Ramos cells from the untreated cells and also from other samples used in this panel. Also, expression of this gene in stimulated Ramos B cells suggests that this gene may be involved in rheumatic disease including rheumatoid arthritis, lupus, osteoarthritis, and hyperproliferative B cell disorders.

In addition, low but significant expression of this gene is also seen in colon and kidney (CTs=32). Furthermore, expression of this gene is decreased in colon samples from patients with IBD colitis and Crohn's disease relative to normal colon. Therefore, therapeutic modulation of the activity of this gene product may be useful in the treatment of inflammatory bowel disease and kidney related disease such as lupus and glomerulonephritis.

Panel 4D Summary: Ag2552 Three experiments with the same probe and primer produce results that are in excellent agreement, with highest expression of the CG93443-01 gene in KU-812 (Basophil) cells (CTs=26-27). This gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

### T. NOV27a (CG50838-01: LEUCINE-RICH REPEAT TRANSMEMBRANE PROTEIN FLRT3)

Expression of gene CG50838-01 was assessed using the primer-probe sets Ag92, Ag2763 and Ag92b, described in Tables TA, TB and TC. Results of the RTQ-PCR runs are shown in Tables TD, TE, TF, TG, TH, TI and TJ.

TABLE TA
Probe Name Ag92

| | | | | Start |
|---|---|---|---|---|
| Primers | Sequences | | Length | Position |
| Forward | 5′-ggctaaatcctgtccatctgtgt-3′ | (SEQ ID NO:284) | 23 | 538 |
| Probe | TET-5′-atgaaacccgcatcgcagcga-3′-TAMRA | (SEQ ID NO:285) | 21 | 562 |
| Reverse | 5′-aatggatgtcagaaagcgatca-3′ | (SEQ ID NO:286) | 22 | 592 |

TABLE TB
Probe Name Ag2763

| | | | | Start |
|---|---|---|---|---|
| Primers | Sequences | | Length | Position |
| Forward | 5′-gcttggatgataatcgcatatc-3′ | (SEQ ID NO:287) | 22 | 999 |
| Probe | TET-5′-ttcatcaccatctcttcaaggtcatca-3′-TAMRA | (SEQ ID NO:288) | 26 | 1027 |
| Reverse | 5′-tgattgttcaacaggtttccat-3′ | (SEQ ID NO:289) | 22 | 1077 |

TABLE TC
Probe Name Ag92b

| | | | | Start |
|---|---|---|---|---|
| Primer | Sequences | | Length | Position |
| Forward | 5′-gcagttagcatagaagagggagca-3′ | (SEQ ID NO:290) | 24 | 881 |
| Probe | TET-5′-tccgagacagcaactatctccgactgc-3′-TAMRA | (SEQ ID NO:291) | 27 | 906 |
| Reverse | 5′-taagqtgattacgggacaggaaa-3′ | (SEQ ID NO:292) | 23 | 934 |

TABLE TD
AI_comprehensive panel_v1.0

| | Rel. Exp.(%) Ag92, | | Rel. Exp.(%) Ag92, |
|---|---|---|---|
| Tissue Name | Run 228157507 | Tissue Name | Run 228157507 |
| 110967 COPD-F | 6.3 | 112427 Match Control | 31.0 |
| | | Psoriasis-F | |
| 110980 COPD-F | 1.6 | 112418 Psoriasis-M | 4.5 |
| 110968 COPD-M | 3.9 | 112723 Match Control | 35.6 |
| | | Psoriasis-M | |
| 110977 COPD-M | 6.7 | 112419 Psoriasis-M | 8.0 |
| 110989 Emphysema-F | 10.1 | 112424 Match Control | 6.4 |
| | | Psoriasis-M | |
| 110992 Emphysema-F | 4.1 | 112420 Psoriasis-M | 14.6 |
| 110993 Emphysema-F | 4.7 | 112425 Match Control | 25.7 |
| | | Psoriasis-M | |
| 110994 Ernphysema-F | 2.2 | 104689 (MF) OA Bone- | 9.8 |
| | | Backus | |
| 110995 Emphysema-F | 9.0 | 104690 (MF) Adj | 2.8 |
| | | “Normal” Bone-Backus | |
| 110996 Emphysema-F | 2.5 | 104691 (MF) OA | 0.6 |
| | | Synovium-Backus | |
| 110997 Asthma-M | 3.6 | 104692 (BA) OA | 0.4 |
| | | Cartilage-Backus | |
| 111001 Asthma-F | 12.5 | 104694 (BA) OA Bone- | 11.0 |
| | | Backus | |
| 111002 Asthma-F | 12.7 | 104695 (BA) Adj | 2.7 |
| | | “Normal” Bone-Backus | |
| 111003 Atopic | 10.0 | 104696 (BA) OA | 0.4 |
| Asthma-F | | Synovium-Backus | |
| 111004 Atopic | 8.7 | 104700 (SS) OA Bone- | 8.0 |
| Asthma-F | | Backus | |
| 111005 Atopic | 8.1 | 104701 (SS) Adj | 5.3 |
| Asthma-F | | “Normal” Bone-Backus | |
| 111006 Atopic | 2.6 | 104702 (SS) OA | 1.1 |
| Asthma-F | | Synovium-Backus | |
| 111417 Allergy-M | 9.7 | 117093 OA Cartilage | 7.7 |
| | | Rep7 | |
| 1112347 Allergy-M | 5.2 | 112672 OA Bone5 | 7.2 |
| 112349 Normal Lung-F | 6.0 | 112673 OA Synovium5 | 2.7 |
| 112357 Normal Lung-F | 35.4 | 112674 OA Synovial | 2.4 |
| | | Fluid cells5 | |
| 112354 Normal Lung-M | 14.0 | 117100 OA Cartilage | 1.5 |
| | | Rep14 | |
| 112374 Crohns-F | 4.8 | 112756 OA Bone9 | 9.7 |
| 112389 Match Control | 3.5 | 112757 OA Synovium9 | 1.4 |
| Crohns-F | | | |
| 112375 Crohns-F | 3.7 | 112758 OA Synovial | 6.5 |
| | | Fluid Cells9 | |
| 112732 Match Control | 0.1 | 117125 RA Cartilage | 4.0 |
| Crohns-F | | Rep2 | |
| 112725 Crohns-M | 6.9 | 113492 Bone2 RA | 87.1 |
| 112387 Match Control | 2.0 | 113493 Synovium2 RA | 38.4 |
| Crohns-M | | | |
| 112378 Crohns-M | 11.4 | 113494 Syn | 64.6 |
| | | Fluid Cells RA | |
| 112390 Match Control | 16.7 | 113499 Cartilage4 RA | 88.9 |
| Crohns-M | | | |
| 112726 Crohns-M | 5.8 | 113500 Bone4 RA | 83.5 |
| 112731 Match Control | 6.3 | 113501 Synovium4 RA | 55.1 |
| Crohns-M | | | |
| 112380 Ulcer Col-F | 11.7 | 113502 Syn Fluid Cells4 | 51.4 |
| | | RA | |
| 112734 Match Control | 1.5 | 113495 Cartilage3 RA | 79.6 |
| Ulcer Col-F | | | |
| 112384 Ulcer Col-F | 10.1 | 113496 Bone3 RA | 94.6 |
| 112737 Match Control | 2.7 | 113497 Synovium3 RA | 47.0 |
| Ulcer Col-F | | | |
| 112386 Ulcer Col-F | 2.5 | 113498 Syn Fluid Cells3 | 100.0 |
| | | RA | |
| 112738 Match Control | 2.8 | 117106 Normal | 1.1 |
| Ulcer Col-F | | Cartilage Rep20 | |
| 112381 Ulcer Col-M | 2.4 | 113663 Bone3 Normal | 6.1 |
| 112735 Match Control | 14.6 | 113664 Synovium3 | 2.9 |
| Ulcer Col-M | | Normal | |
| 112382 Ulcer Col-M | 5.4 | 113665 Syn Fluid Cells3 | 4.5 |
| | | Normal | |
| 112394 Match Control | 1.6 | 117107 Normal | 2.8 |
| Ulcer Col-M | | Cartilage Rep22 | |
| 112383 Ulcer Col-M | 6.4 | 113667 Bone4 Normal | 2.9 |
| 112736 Match Control | 1.9 | 113668 Synovium4 | 3.4 |
| Ulcer Col-M | | Normal | |
| 112423 Psoriasis-F | 11.0 | 113669 Syn Fluid Cells4 | 2.9 |
| | | Normal | |

TABLE TE
CNS_neurodegeneration_v1.0

| | Rel. | | Rel. | | Rel. | | Rel. |
|---|---|---|---|---|---|---|---|
| | Exp.(%) | Rel. | Exp.(%) | | Exp.(%) | Rel. | Exp.(%) |
| | Ag2763, | Exp.(%) | Ag92b, | | Ag2763, | Exp.(%) | Ag92b, |
| Tissue | Run | Ag92, Run | Run | Tissue | Run | Ag92, Run | Run |
| Name | 206985755 | 206974451 | 225000617 | Name | 206985755 | 206974451 | 225000617 |
| AD 1 | 2.5 | 7.8 | 12.4 | Control | 0.5 | 2.4 | 1.4 |
| Hippo | | | | (Path) 3 | | | |
| | | | | Temporal | | | |
| | | | | Ctx | | | |
| AD 2 | 4.0 | 9.8 | 13.3 | Control | 8.4 | 26.2 | 26.8 |
| Hippo | | | | (Path) 4 | | | |
| | | | | Temporal | | | |
| | | | | Ctx | | | |
| AD 3 | 0.8 | 3.1 | 4.1 | AD 1 | 4.8 | 18.4 | 16.2 |
| Hippo | | | | Occipital | | | |
| | | | | Ctx | | | |
| AD 4 | 0.5 | 3.1 | 2.8 | AD 2 | 0.0 | 0.0 | 0.0 |
| Hippo | | | | Occipital | | | |
| | | | | Ctx | | | |
| | | | | (Missing) | | | |
| AD 5 | 37.6 | 100.0 | 100.0 | AD 3 | 0.7 | 2.4 | 3.0 |
| Hippo | | | | Occipital | | | |
| | | | | Ctx | | | |
| AD 6 | 15.2 | 33.9 | 28.1 | AD 4 | 3.3 | 13.5 | 11.5 |
| Hippo | | | | Occipital | | | |
| | | | | Ctx | | | |
| Control 2 | 7.4 | 23.2 | 15.0 | AD 5 | 18.6 | 46.0 | 12.9 |
| Hippo | | | | Occipital | | | |
| | | | | Ctx | | | |
| Control 4 | 0.9 | 2.1 | 4.5 | AD 6 | 6.2 | 15.4 | 35.8 |
| Hippo | | | | Occipital | | | |
| | | | | Ctx | | | |
| Control | 0.9 | 3.4 | 3.3 | Control 1 | 0.1 | 0.9 | 1.5 |
| (Path) 3 | | | | Occipital | | | |
| Hippo | | | | Ctx | | | |
| AD 1 | 1.9 | 8.7 | 7.6 | Control 2 | 40.1 | 68.3 | 65.1 |
| Temporal | | | | Occipital | | | |
| Ctx | | | | Ctx | | | |
| AD 2 | 7.9 | 23.3 | 26.8 | Control 3 | 5.4 | 13.4 | 21.9 |
| Temporal | | | | Occipital | | | |
| Ctx | | | | Ctx | | | |
| AD 3 | 100.0 | 2.4 | 1.7 | Control 4 | 0.7 | 2.1 | 1.2 |
| Temporal | | | | Occipital | | | |
| Ctx | | | | Ctx | | | |
| AD 4 | 3.2 | 12.9 | 14.2 | Control | 28.5 | 69.3 | 58.6 |
| Temporal | | | | (Path) 1 | | | |
| Ctx | | | | Occipital | | | |
| | | | | Ctx | | | |
| AD 5 Inf | 28.5 | 75.3 | 14.3 | Control | 3.3 | 10.8 | 12.9 |
| Temporal | | | | (Path) 2 | | | |
| Ctx | | | | Occipital | | | |
| | | | | Ctx | | | |
| AD 5 Sup | 8.9 | 21.2 | 29.5 | Control | 0.1 | 1.5 | 0.9 |
| Temporal | | | | (Path) 3 | | | |
| Ctx | | | | Occipital | | | |
| | | | | Ctx | | | |
| AD 6 Inf | 9.2 | 27.9 | 27.7 | Control | 5.3 | 17.4 | 13.1 |
| Temporal | | | | (Path) 4 | | | |
| Ctx | | | | Occipital | | | |
| | | | | Ctx | | | |
| AD 6 Sup | 12.0 | 31.4 | 14.1 | Control 1 | 0.5 | 1.6 | 1.6 |
| Temporal | | | | Parietal | | | |
| Ctx | | | | Ctx | | | |
| Control 1 | 0.3 | 2.0 | 1.9 | Control 2 | 9.9 | 23.3 | 27.5 |
| Temporal | | | | Parietal | | | |
| Ctx | | | | Ctx | | | |
| Control 2 | 16.5 | 25.7 | 28.3 | Control 3 | 3.8 | 11.9 | 14.0 |
| Temporal | | | | Parietal | | | |
| Ctx | | | | Ctx | | | |
| Control 3 | 2.8 | 11.3 | 13.6 | Control | 26.1 | 50.0 | 45.7 |
| Temporal | | | | (Path) 1 | | | |
| Ctx | | | | Parietal | | | |
| | | | | Ctx | | | |
| Control 3 | 1.0 | 4.3 | 3.8 | Control | 5.6 | 13.5 | 9.8 |
| Temporal | | | | (Path) 2 | | | |
| Ctx | | | | Parietal | | | |
| | | | | Ctx | | | |
| Control | 18.9 | 37.9 | 25.7 | Control | 0.2 | 1.7 | 1.5 |
| (Path) 1 | | | | (Path) 3 | | | |
| Temporal | | | | Parietal | | | |
| Ctx | | | | Ctx | | | |
| Control | 8.0 | 26.4 | 26.2 | Control | 13.2 | 39.5 | 14.5 |
| (Path) 2 | | | | (Path) 4 | | | |
| Temporal | | | | Parietal | | | |
| Ctx | | | | Ctx | | | |

TABLE 1F
Panel I

| | Rel. | | Rel. | | Rel. | | Rel. |
|---|---|---|---|---|---|---|---|
| | Exp.(%) | Rel. | Exp.(%) | | Exp.(%) | Rel. | Exp.(%) |
| | Ag92, | Exp.(%) | Ag92b, | | Ag92, | Exp.(%) | Ag92b, |
| | Run | Ag92, Run | Run | Tissue | Run | Ag92, Run | Run |
| Tissue Name | 87586687 | 124961889 | 87584661 | Name | 87586687 | 124961889 | 87584661 |
| Endothelial | 0.0 | 0.0 | 0.1 | Renal ca. | 0.5 | 0.0 | 1.0 |
| cells | | | | 786-0 | | | |
| Endothelial | 0.1 | 0.0 | 0.2 | Renal ca. | 0.4 | 0.0 | 0.9 |
| cells (treated) | | | | A498 | | | |
| Pancreas | 1.6 | 0.0 | 1.9 | Renal ca. | 0.4 | 0.0 | 0.9 |
| | | | | RXF 393 | | | |
| Pancreatic ca. | 3.3 | 0.0 | 4.2 | Renal ca. | 0.9 | 0.0 | 2.7 |
| CAPAN 2 | | | | ACHN | | | |
| Adrenal gland | 0.9 | 0.0 | 1.1 | Renal ca. | 0.8 | 0.0 | 1.8 |
| | | | | UO-31 | | | |
| Thyroid | 4.1 | 0.0 | 4.6 | Renal ca. | 7.8 | 0.7 | 22.1 |
| | | | | TK-10 | | | |
| Salivary gland | 0.2 | 0.0 | 0.8 | Liver | 2.6 | 0.0 | 3.9 |
| Pituitary gland | 0.6 | 0.0 | 0.5 | Liver (fetal) | 2.9 | 0.0 | 6.7 |
| Brain (fetal) | 2.6 | 0.0 | 3.0 | Liver ca. | 0.0 | 0.0 | 0.0 |
| | | | | (hepatoblast) | | | |
| | | | | HepG2 | | | |
| Brain (whole) | 16.3 | 27.7 | 21.5 | Lung | 3.1 | 19.9 | 5.5 |
| Brain | 0.4 | 0.0 | 4.6 | Lung (fetal) | 10.7 | 3.0 | 21.2 |
| (amygdala) | | | | | | | |
| Brain | 100.0 | 100.0 | 100.0 | Lung ca. | 0.9 | 0.0 | 3.8 |
| (cerebellum) | | | | (small cell) | | | |
| | | | | LX-1 | | | |
| Brain | 12.2 | 0.0 | 10.1 | Lung ca. | 5.3 | 0.0 | 8.2 |
| (hippocampus) | | | | (small cell) | | | |
| | | | | NCI-H69 | | | |
| Brain | 0.2 | 0.0 | 2.5 | Lung ca. | 0.0 | 0.0 | 2.9 |
| (substantia | | | | (s.cell var.) | | | |
| nigra) | | | | SHP-77 | | | |
| Brain | 2.9 | 0.0 | 6.2 | Lung ca. | 0.0 | 4.1 | 3.5 |
| (thalamus) | | | | (large | | | |
| | | | | cell)NCI- | | | |
| | | | | H460 | | | |
| Brain | 1.5 | 0.0 | 2.6 | Lung ca. | 5.8 | 0.4 | 13.7 |
| (hypothalamus) | | | | (non-sm. | | | |
| | | | | cell) A549 | | | |
| Spinal cord | 1.1 | 0.0 | 4.5 | Lung ca. | 0.3 | 0.0 | 0.2 |
| | | | | (non-s.cell) | | | |
| | | | | NCI-H23 | | | |
| glio/astro U87- | 0.1 | 0.0 | 0.1 | Lung ca. | 2.7 | 0.0 | 3.9 |
| MG | | | | (non-s.cell) | | | |
| | | | | HOP-62 | | | |
| glio/astro U- | 0.1 | 0.0 | 0.1 | Lung ca. | 1.6 | 0.0 | 4.2 |
| 118-MG | | | | (non-s.cl) | | | |
| | | | | NCI-H522 | | | |
| astrocytoma | 0.2 | 0.0 | 0.5 | Lung ca. | 4.3 | 0.0 | 7.0 |
| SW1783 | | | | (squam.) SW | | | |
| | | | | 900 | | | |
| neuro*; met | 1.1 | 0.0 | 2.7 | Lung ca. | 6.0 | 0.0 | 5.5 |
| SK-N-AS | | | | (squam.) | | | |
| | | | | NCI-H596 | | | |
| astrocytoma | 0.0 | 0.0 | 0.0 | Mammary | 2.5 | 0.0 | 6.7 |
| SF-539 | | | | gland | | | |
| astrocytoma | 9.5 | 5.0 | 23.2 | Breast ca.* | 4.5 | 0.0 | 3.7 |
| SNB-75 | | | | (pl.ef) | | | |
| | | | | MCF-7 | | | |
| glioma SNB-19 | 4.4 | 0.0 | 10.6 | Breast ca.* | 0.0 | 0.0 | 0.0 |
| | | | | (pl.ef) | | | |
| | | | | MDA-MB- | | | |
| | | | | 231 | | | |
| glioma U251 | 0.0 | 0.0 | 0.1 | Breast ca.* | 0.1 | 0.0 | 1.8 |
| | | | | (pl. ef) | | | |
| | | | | T47D | | | |
| glioma SF-295 | 0.0 | 0.0 | 0.0 | Breast ca. | 0.0 | 0.0 | 2.0 |
| | | | | BT-549 | | | |
| Heart | 0.5 | 0.0 | 0.6 | Breast ca. | 1.5 | 0.0 | 2.1 |
| | | | | MDA-N | | | |
| Skeletal muscle | 2.2 | 0.0 | 1.9 | Ovary | 0.9 | 0.0 | 3.0 |
| Bone marrow | 0.0 | 0.0 | 0.2 | Ovarian ca. | 0.8 | 0.0 | 0.9 |
| | | | | OVCAR-3 | | | |
| Thymus | 13.8 | 0.0 | 15.2 | Ovarian ca. | 0.6 | 0.0 | 0.7 |
| | | | | OVCAR-4 | | | |
| Spleen | 0.0 | 0.0 | 0.4 | Ovarian ca. | 16.3 | 9.6 | 28.3 |
| | | | | OVCAR-5 | | | |
| Lymph node | 0.2 | 0.0 | 0.6 | Ovarian ca. | 0.6 | 0.0 | 0.3 |
| | | | | OVCAR-8 | | | |
| Colon | 3.4 | 0.0 | 3.6 | Ovarian ca. | 6.9 | 0.1 | 15.2 |
| (ascending) | | | | IGROV-1 | | | |
| Stomach | 13.1 | 0.1 | 14.9 | Ovarian ca. | 2.8 | 0.0 | 7.0 |
| | | | | (ascites) SK- | | | |
| | | | | OV-3 | | | |
| Small intestine | 1.2 | 0.0 | 2.6 | Uterus | 10.2 | 18.8 | 19.5 |
| Colon ca. | 0.1 | 0.0 | 0.1 | Placenta | 1.6 | 0.0 | 2.5 |
| SW480 | | | | | | | |
| Colon ca.* | 0.0 | 0.0 | 0.1 | Prostate | 6.4 | 0.0 | 11.3 |
| SW620 | | | | | | | |
| (SW480 met) | | | | | | | |
| Colon ca. | 1.0 | 0.0 | 1.4 | Prostate ca.* | 0.0 | 0.2 | 1.4 |
| HT29 | | | | (bone met) | | | |
| | | | | PC-3 | | | |
| Colon ca. | 0.0 | 0.0 | 0.2 | Testis | 22.2 | 25.5 | 19.1 |
| HCT-116 | | | | | | | |
| Colon ca. | 20.9 | 25.0 | 45.7 | Melanoma | 0.2 | 0.0 | 0.3 |
| CaCo-2 | | | | Hs688(A).T | | | |
| Colon ca. | 0.8 | 0.0 | 0.4 | Melanoma* | 0.1 | 0.0 | 0.4 |
| HCT-15 | | | | (met) | | | |
| | | | | Hs688(B).T | | | |
| Colon ca. | 0.4 | 0.0 | 1.6 | Melanoma | 1.3 | 0.0 | 0.9 |
| HCC-2998 | | | | UACC-62 | | | |
| Gastric ca.* | 19.9 | 36.3 | 46.7 | Melanoma | 1.3 | 0.0 | 0.7 |
| (liver met) | | | | M14 | | | |
| NCI-N87 | | | | | | | |
| Bladder | 2.0 | 0.1 | 3.1 | Melanoma | 0.1 | 0.0 | 0.5 |
| | | | | LOX IMVI | | | |
| Trachea | 4.5 | 0.0 | 7.6 | Melanoma* | 0.6 | 0.0 | 0.3 |
| | | | | (met) SK- | | | |
| | | | | MEL-5 | | | |
| Kidney | 7.7 | 0.0 | 19.2 | Melanoma | 1.0 | 0.0 | 1.2 |
| | | | | SK-MEL-28 | | | |
| Kidney (fetal) | 20.7 | 3.9 | 31.2 | | | | |

TABLE TG
Panel 1.3D

| | Rel. Exp.(%) | Rel. Exp.(%) | | Rel. Exp.(%) | Rel. Exp.(%) |
|---|---|---|---|---|---|
| | Ag2763, Run | Ag92b, Run | | Ag2763, Run | Ag92b, Run |
| Tissue Name | 165527214 | 165974816 | Tissue Name | 165527214 | 165974816 |
| Liver | 4.1 | 3.8 | Kidney (fetal) | 35.1 | 51.8 |
| adenocarcinoma | | | | | |
| Pancreas | 1.5 | 1.7 | Renal ca. 786-0 | 2.0 | 2.6 |
| Pancreatic ca. | 14.0 | 25.9 | Renal ca. A498 | 3.7 | 3.1 |
| CAPAN 2 | | | | | |
| Adrenal gland | 0.9 | 4.3 | Renal ca. RXF | 4.0 | 4.1 |
| | | | 393 | | |
| Thyroid | 5.6 | 4.5 | Renal ca. | 3.2 | 9.3 |
| | | | ACHN | | |
| Salivary gland | 1.5 | 1.1 | Renal ca. UO- | 10.5 | 18.4 |
| | | | 31 | | |
| Pituitary gland | 4.3 | 6.3 | Renal ca. TK- | 22.1 | 33.9 |
| | | | 10 | | |
| Brain (fetal) | 20.4 | 20.6 | Liver | 3.1 | 4.8 |
| Brain (whole) | 47.3 | 95.3 | Liver (fetal) | 18.7 | 20.7 |
| Brain (amygdala) | 24.0 | 38.2 | Liver ca. | 0.0 | 0.0 |
| | | | (hepatoblast) | | |
| | | | HepG2 | | |
| Brain (cerebellum) | 49.0 | 64.6 | Lung | 23.7 | 44.1 |
| Brain | 28.3 | 36.1 | Lung (fetal) | 18.0 | 50.0 |
| (hippocampus) | | | | | |
| Brain (substantia | 3.8 | 6.8 | Lung ca. | 7.1 | 15.4 |
| nigra) | | | (small cell) | | |
| | | | LX-1 | | |
| Brain (thalamus) | 32.1 | 45.1 | Lung ca. | 6.1 | 43.8 |
| | | | (small cell) | | |
| | | | NCI-H69 | | |
| Cerebral Cortex | 15.7 | 26.2 | Lung ca. (s.cell | 4.7 | 3.7 |
| | | | var.) SHP-77 | | |
| Spinal cord | 6.2 | 22.4 | Lung ca. (large | 9.3 | 3.8 |
| | | | cell)NCI-H460 | | |
| glio/astro U87-MG | 0.6 | 0.0 | Lung ca. (non- | 14.1 | 19.8 |
| | | | sm. cell) A549 | | |
| glio/astro U-118- | 1.2 | 0.6 | Lung ca. (non- | 0.0 | 0.8 |
| MG | | | s.cell) NCI- | 0.0 | 0.8 |
| | | | H23 | | |
| astrocytoma | 0.9 | 4.7 | Lung ca. (non- | 10.5 | 16.2 |
| SW1783 | | | s.cell) HOP-62 | | |
| neuro*; met SK-N- | 6.6 | 5.6 | Lung ca. (non- | 2.0 | 2.9 |
| AS | | | s.cl) NCI-H522 | | |
| astrocytoma SF- | 0.0 | 1.7 | Lung ca. | 3.5 | 12.4 |
| 539 | | | (squam.) SW | | |
| | | | 900 | | |
| astrocytoma SNB- | 15.6 | 11.3 | Lung ca. | 9.0 | 15.9 |
| 75 | | | (squam.) NCI- | | |
| | | | H596 | | |
| glioma SNB-19 | 12.7 | 39.0 | Mammary | 2.8 | 1.2 |
| | | | gland | | |
| glioma U251 | 0.3 | 0.0 | Breast ca.* | 3.1 | 3.5 |
| | | | (pl.ef) MCF-7 | | |
| glioma SF-295 | 0.0 | 0.0 | Breast ca.* | 0.2 | 0.0 |
| | | | (pl.ef) MDA- | | |
| | | | MB-231 | | |
| Heart (fetal) | 0.3 | 0.3 | Breast ca.* | 1.1 | 2.9 |
| | | | (pl.ef) T47D | | |
| Heart | 1.6 | 0.6 | Breast ca. BT- | 6.7 | 2.9 |
| | | | 549 | | |
| Skeletal muscle | 3.4 | 1.0 | Breast ca. | 0.8 | 2.8 |
| (fetal) | | | MDA-N | | |
| Skeletal muscle | 8.2 | 9.9 | Ovary | 0.4 | 1.2 |
| Bone marrow | 0.0 | 2.4 | Ovarian ca. | 3.2 | 3.2 |
| | | | OVCAR-3 | | |
| Thymus | 2.6 | 6.9 | Ovarian ca. | 1.8 | 7.7 |
| | | | OVCAR-4 | | |
| Spleen | 0.4 | 0.6 | Ovarian ca. | 48.6 | 100.0 |
| | | | OVCAR-5 | | |
| Lymph node | 0.3 | 4.0 | Ovarian ca. | 1.5 | 0.5 |
| | | | OVCAR-8 | | |
| Colorectal | 1.7 | 2.4 | Ovarian ca. | 22.4 | 88.3 |
| | | | IGROV-1 | | |
| Stomach | 11.6 | 11.9 | Ovarian ca.* | 6.8 | 34.2 |
| | | | (ascites) SK- | | |
| | | | OV-3 | | |
| Small intestine | 9.6 | 2.5 | Uterus | 2.2 | 2.0 |
| Colon ca. SW480 | 0.3 | 1.4 | Placenta | 1.1 | 8.5 |
| Colon ca.* | 0.8 | 4.1 | Prostate | 12.2 | 4.7 |
| SW620(SW480 | | | | | |
| met) | | | | | |
| Colon ca. HT29 | 1.6 | 1.9 | Prostate ca.* | 1.6 | 0.0 |
| | | | (bone met)PC-3 | | |
| Colon ca. HCT- | 0.4 | 0.5 | Testis | 2.6 | 2.5 |
| 116 | | | | | |
| Colon ca. CaCo-2 | 34.6 | 92.7 | Melanoma | 0.5 | 0.9 |
| | | | Hs688(A).T | | |
| Colon ca. | 1.9 | 6.2 | Melanoma* | 0.3 | 0.7 |
| tissue(ODO3866) | | | (met) | | |
| | | | Hs688(B).T | | |
| Colon ca. HCC- | 4.2 | 1.4 | Melanoma | 7.2 | 9.9 |
| 2998 | | | UACC-62 | | |
| Gastric ca.* (liver | 100.0 | 79.0 | Melanoma | 2.6 | 1.7 |
| met) NCI-N87 | | | M14 | | |
| Bladder | 11.2 | 19.8 | Melanoma | 0.0 | 0.0 |
| | | | LOX IMVI | | |
| Trachea | 3.8 | 5.9 | Melanoma* | 0.4 | 0.0 |
| | | | (met) SK- | | |
| | | | MEL-5 | | |
| Kidney | 27.7 | 33.2 | Adipose | 9.0 | 19.6 |

TABLE TH
Panel 2D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag2763, | Ag92, | Ag92b, | | Ag2763, | Ag92, | Ag92b, |
| Tissue | Run | Run | Run | Tissue | Run | Run | Run |
| Name | 162555842 | 149912056 | 151275585 | Name | 162555842 | 149912056 | 151275585 |
| Normal | 9.7 | 7.0 | 7.5 | Kidney | 5.1 | 6.8 | 4.4 |
| Colon | | | | Margin | | | |
| | | | | 8120608 | | | |
| CC Well to | 0.7 | 0.5 | 0.7 | Kidney | 0.2 | 0.2 | 0.1 |
| Mod Diff | | | | Cancer | | | |
| (ODO3866) | | | | 8120613 | | | |
| CC Margin | 1.2 | 1.3 | 0.7 | Kidney | 3.4 | 3.1 | 3.3 |
| (ODO3866) | | | | Margin | | | |
| | | | | 8120614 | | | |
| CC Gr.2 | 1.0 | 0.5 | 0.9 | Kidney | 2.6 | 4.2 | 2.5 |
| rectosigmoid | | | | Cancer | | | |
| (ODO3868) | | | | 9010320 | | | |
| CC Margin | 0.6 | 0.8 | 0.6 | Kidney | 11.0 | 13.7 | 15.3 |
| (ODO3868) | | | | Margin | | | |
| | | | | 9010321 | | | |
| CC Mod | 1.0 | 0.5 | 1.0 | Normal | 2.2 | 1.2 | 0.9 |
| Diff | | | | Uterus | | | |
| (ODO3920) | | | | | | | |
| CC Margin | 2.1 | 1.5 | 2.2 | Uterus | 4.5 | 3.5 | 3.8 |
| (ODO3920) | | | | Cancer | | | |
| | | | | 064011 | | | |
| CC Gr.2 | 4.5 | 2.0 | 2.5 | Normal | 3.2 | 2.7 | 2.3 |
| ascend colon | | | | Thyroid | | | |
| (ODO3921) | | | | | | | |
| CC Margin | 2.2 | 1.6 | 1.3 | Thyroid | 7.9 | 21.9 | 16.5 |
| (ODO3921) | | | | Cancer | | | |
| | | | | 064010 | | | |
| CC from | 4.4 | 4.1 | 3.1 | Thyroid | 10.4 | 12.7 | 10.6 |
| Partial | | | | Cancer | | | |
| Hepatectomy | | | | A302152 | | | |
| (ODO4309) | | | | | | | |
| Mets | | | | | | | |
| Liver Margin | 4.9 | 3.0 | 4.1 | Thyroid | 8.5 | 6.0 | 4.8 |
| (ODO4309) | | | | Margin | | | |
| | | | | A302153 | | | |
| Colon mets | 3.1 | 2.2 | 3.1 | Normal | 5.2 | 4.1 | 3.2 |
| to lung | | | | Breast | | | |
| (OD04451- | | | | | | | |
| 01) | | | | | | | |
| Lung Margin | 12.3 | 11.3 | 9.7 | Breast | 0.9 | 0.7 | 0.7 |
| (OD04451- | | | | Cancer | | | |
| 02) | | | | (OD04566) | | | |
| Normal | 42.0 | 5.9 | 6.2 | Breast | 2.9 | 2.5 | 2.0 |
| Prostate | | | | Cancer | | | |
| 6546-1 | | | | (OD04590- | | | |
| | | | | 01) | | | |
| Prostate | 6.7 | 6.4 | 5.4 | Breast | 2.1 | 1.8 | 1.4 |
| Cancer | | | | Cancer | | | |
| (OD04410) | | | | Mets | | | |
| | | | | (OD04590- | | | |
| | | | | 03) | | | |
| Prostate | 10.4 | 7.7 | 8.2 | Breast | 44.8 | 50.3 | 30.4 |
| Margin | | | | Cancer | | | |
| (OD04410) | | | | Metastasis | | | |
| | | | | (OD04655- | | | |
| | | | | 05) | | | |
| Prostate | 15.9 | 13.3 | 12.6 | Breast | 1.5 | 1.2 | 1.2 |
| Cancer | | | | Cancer | | | |
| (OD04720- | | | | 064006 | | | |
| 01) | | | | | | | |
| Prostate | 35.8 | 27.7 | 30.6 | Breast | 2.0 | 2.1 | 1.8 |
| Margin | | | | Cancer | | | |
| (OD04720- | | | | 1024 | | | |
| 02) | | | | | | | |
| Normal | 30.6 | 21.5 | 19.9 | Breast | 3.9 | 2.1 | 2.1 |
| Lung 061010 | | | | Cancer | | | |
| | | | | 9100266 | | | |
| Lung Met to | 0.5 | 0.4 | 0.3 | Breast | 0.9 | 0.9 | 1.1 |
| Muscle | | | | Margin | | | |
| (ODO4286) | | | | 9100265 | | | |
| Muscle | 1.1 | 0.3 | 0.3 | Breast | 8.7 | 5.6 | 5.6 |
| Margin | | | | Cancer | | | |
| (ODO4286) | | | | A209073 | | | |
| Lung | 14.7 | 10.4 | 9.3 | Breast | 2.6 | 3.4 | 2.6 |
| Malignant | | | | Margin | | | |
| Cancer | | | | A209073 | | | |
| (OD03126) | | | | | | | |
| Lung Margin | 80.1 | 56.6 | 53.6 | Normal | 1.7 | 1.2 | 1.3 |
| (OD03126) | | | | | | | |
| Lung Cancer | 19.1 | 22.1 | 15.6 | Liver | 0.1 | 0.4 | 0.5 |
| (OD04404) | | | | Cancer | | | |
| | | | | 064003 | | | |
| Lung Margin | 26.8 | 36.6 | 22.1 | Liver | 1.5 | 1.7 | 1.3 |
| (OD04404) | | | | Cancer | | | |
| | | | | 1025 | | | |
| Lung Cancer | 6.1 | 4.9 | 5.0 | Liver | 0.9 | 0.5 | 0.6 |
| (OD04565) | | | | Cancer | | | |
| | | | | 1026 | | | |
| Lung Margin | 14.7 | 19.2 | 15.5 | Liver | 1.5 | 2.4 | 1.6 |
| (OD04565) | | | | Cancer | | | |
| | | | | 6004-T | | | |
| Lung Cancer | 5.6 | 5.3 | 4.3 | Liver | 0.4 | 0.3 | 0.5 |
| (OD04237- | | | | Tissue | | | |
| 01) | | | | 6004-N | | | |
| Lung Margin | 28.7 | 32.5 | 28.7 | Liver | 0.6 | 0.9 | 0.7 |
| (OD04237- | | | | Cancer | | | |
| 02) | | | | 6005-T | | | |
| Ocular Mel | 0.4 | 0.1 | 0.1 | Liver | 0.2 | 0.2 | 0.3 |
| Met to Liver | | | | Tissue | | | |
| (ODO4310) | | | | 6005-N | | | |
| Liver Margin | 5.8 | 3.5 | 3.6 | Normal | 9.7 | 11.0 | 10.4 |
| (ODO4310) | | | | Bladder | | | |
| Melanoma | 6.0 | 5.0 | 4.7 | Bladder | 0.2 | 0.2 | 0.5 |
| Mets to | | | | Cancer | | | |
| Lung | | | | 1023 | | | |
| (OD04321) | | | | | | | |
| Lung Margin | 62.9 | 57.8 | 38.7 | Bladder | 2.0 | 1.8 | 1.4 |
| (OD04321) | | | | Cancer | | | |
| | | | | A302173 | | | |
| Normal | 45.4 | 55.9 | 25.3 | Bladder | 1.2 | 0.7 | 1.2 |
| Kidney | | | | Cancer | | | |
| | | | | (OD04718- | | | |
| | | | | 01) | | | |
| Kidney Ca, | 69.7 | 55.1 | 49.3 | Bladder | 2.7 | 3.4 | 3.5 |
| Nuclear | | | | Normal | | | |
| grade 2 | | | | Adjacent | | | |
| (OD04338) | | | | (OD04718- | | | |
| | | | | 03) | | | |
| Kidney | 21.3 | 24.7 | 19.2 | Normal | 1.2 | 0.9 | 0.9 |
| Margin | | | | Ovary | | | |
| (OD04338) | | | | | | | |
| Kidney Ca | 16.2 | 19.9 | 27.9 | Ovarian | 3.3 | 3.7 | 2.5 |
| Nuclear | | | | Cancer | | | |
| grade 1/2 | | | | 064008 | | | |
| (OD04339) | | | | | | | |
| Kidney | 41.2 | 33.9 | 29.5 | Ovarian | 0.9 | 0.5 | 0.7 |
| Margin | | | | Cancer | | | |
| (OD04339) | | | | (OD04768- | | | |
| | | | | 07) | | | |
| Kidney Ca, | 29.3 | 34.9 | 25.0 | Ovary | 1.0 | 1.6 | 0.9 |
| Clear cell | | | | Margin | | | |
| type | | | | (OD04768- | | | |
| (OD04340) | | | | 08) | | | |
| Kidney | 20.0 | 26.6 | 19.6 | Normal | 6.9 | 7.2 | 7.0 |
| Margin | | | | Stomach | | | |
| (OD04340) | | | | | | | |
| Kidney Ca, | 0.6 | 0.3 | 0.4 | Gastric | 0.5 | 0.7 | 0.5 |
| Nuclear | | | | Cancer | | | |
| grade 3 | | | | 9060358 | | | |
| (OD04348) | | | | | | | |
| Kidney | 30.8 | 20.9 | 21.5 | Stomach | 3.7 | 2.8 | 2.5 |
| Margin | | | | Margin | | | |
| (OD04348) | | | | 9060359 | | | |
| Kidney | 0.7 | 0.3 | 0.6 | Gastric | 6.3 | 6.9 | 5.6 |
| Cancer | | | | Cancer | | | |
| (OD04622- | | | | 9060395 | | | |
| 01) | | | | | | | |
| Kidney | 2.9 | 2.9 | 4.5 | Stomach | 2.4 | 3.6 | 2.5 |
| Margin | | | | Margin | | | |
| (OD04622- | | | | 9060394 | | | |
| 03) | | | | | | | |
| Kidney | 100.0 | 100.0 | 100.0 | Gastric | 5.1 | 4.3 | 3.8 |
| Cancer | | | | Cancer | | | |
| (OD04450- | | | | 9060397 | | | |
| 01) | | | | | | | |
| Kidney | 36.1 | 29.3 | 24.1 | Stomach | 1.5 | 0.9 | 1.3 |
| Margin | | | | Margin | | | |
| (OD04450- | | | | 9060396 | | | |
| 03) | | | | | | | |
| Kidney | 1.8 | 1.5 | 1.3 | Gastric | 10.7 | 7.0 | 10.4 |
| Cancer | | | | Cancer | | | |
| 8120607 | | | | 064005 | | | |

TABLE TI
Panel 3D

| | Rel. Exp.(%) | | Rel. Exp.(%) |
|---|---|---|---|
| | Ag92b, Run | | Ag92b, Run |
| Tissue Name | 165919165 | Tissue Name | 165919165 |
| Daoy-Medulloblastoma | 0.4 | Ca Ski-Cervical epidermoid | 0.1 |
| | | carcinoma (metastasis) | |
| TE671-Medulloblastoma | 0.4 | ES-2-Ovarian clear cell | 0.2 |
| | | carcinoma | |
| D283 Med- | 3.3 | Ramos-Stimulated with | 0.0 |
| Medulloblastoma | | PMA/ionomycin 6h | |
| PFSK-1-Primitive | 15.1 | Ramos-Stimulated with | 0.0 |
| Neuroectodermal | | PMA/ionomycin 14h | |
| XF-498-CNS | 100.0 | MEG-01-Chronic | 0.5 |
| | | myelogenous leukemia | |
| | | (megokaryoblast) | |
| SNB-78-Glioma | 0.1 | Raji-Burkitt's lymphoma | 0.0 |
| SF-268-Glioblastoma | 0.0 | Daudi-Burkitt's lymphoma | 0.1 |
| T98G-Glioblastoma | 1.0 | U266-B-cell plasmacytoma | 0.0 |
| SK-N-SH-Neuroblastoma | 6.3 | CA46-Burkitt's lymphoma | 0.0 |
| (metastasis) | | | |
| SF-295-Glioblastoma | 0.0 | RL-non-Hodgkin's B-cell | 0.0 |
| | | lymphoma | |
| Cerebellum | 41.2 | JM1-pre-B-cell lymphoma | 0.0 |
| Cerebellum | 1.8 | Jurkat-T cell leukemia | 0.0 |
| NCI-H292- | 0.7 | TF-1-Erythroleukemia | 0.1 |
| Mucoepidermoid lung | | | |
| carcinoma | | | |
| DMS-114-Small cell | 0.2 | HUT 78-T-cell lymphoma | 0.0 |
| lung cancer | | | |
| DMS-79-Small cell lung | 0.9 | U937-Histiocytic lymphoma | 0.0 |
| cancer | | | |
| NCI-H146-Small cell | 5.5 | KU-812-Myelogenous | 0.3 |
| lung cancer | | leukemia | |
| NCI-H526-Small cell | 0.8 | 769-P-Clear cell renal | 0.8 |
| lung cancer | | carcinoma | |
| NCI-N417-Small cell | 1.7 | Caki-2-Clear cell renal | 18.8 |
| lung cancer | | carcinoma | |
| NCI-H82-Small cell lung | 3.8 | SW 839-Clear cell renal | 1.2 |
| cancer | | carcinoma | |
| NCI-H157-Squamous | 0.0 | G401-Wilms'tumor | 0.0 |
| cell lung cancer | | | |
| (metastasis) | | | |
| NCI-H1155-Large cell | 0.2 | Hs766T-Pancreatic carcinoma | 0.3 |
| lung cancer | | (LN metastasis) | |
| NCI-H1299-Large cell | 0.2 | CAPAN-1-Pancreatic | 2.0 |
| lung cancer | | adenocarcinoma (liver | |
| | | metastasis) | |
| NCI-H727-Lung | 84.1 | SU86.86-Pancreatic | 1.8 |
| carcinoid | | carcinoma (liver metastasis) | |
| NCI-UMC-11-Lung | 24.3 | BxPC-3-Pancreatic | 6.9 |
| carcinoid | | adenocarcinoma | |
| LX-1-Small cell lung | 5.8 | HPAC-Pancreatic | 6.9 |
| cancer | | adenocarcinoma | |
| Colo-205-Colon cancer | 0.4 | MIA PaCa-2-Pancreatic | 0.1 |
| | | carcinoma | |
| KM12-Colon cancer | 1.2 | CFPAC-1-Pancreatic ductal | 6.0 |
| | | adenocarcinoma | |
| KM20L2-Colon cancer | 1.9 | PANC-1-Pancreatic | 0.4 |
| | | epithelioid ductal carcinoma | |
| NCI-H716-Colon cancer | 40.9 | T24-Bladder carcinma | 4.6 |
| | | (transitional cell) | |
| SW-48-Colon | 0.0 | 5637-Bladder carcinoma | 2.6 |
| adenocarcinoma | | | |
| SW1116-Colon | 1.4 | HT-1197-Bladder carcinoma | 0.0 |
| adenocarcinoma | | | |
| LS 174T-Colon | 1.2 | UM-UC-3-Bladder carcinma | 0.0 |
| adenocarcinoma | | (transitional cell) | |
| SW-948-Colon | 0.2 | A204-Rhabdomyosarcoma | 0.0 |
| adenocarcinoma | | | |
| SW-480-Colon | 0.1 | HT-1080-Fibrosarcoma | 1.2 |
| adenocarcinoma | | | |
| NCI-SNU-5-Gastric | 0.1 | MG-63-Osteosarcoma | 1.4 |
| carcinoma | | | |
| KATO III-Gastric | 17.3 | SK-LMS-1-Leiomyosarcoma | 0.8 |
| carcinoma | | (vulva) | |
| NCI-SNU-16-Gastric | 0.9 | SJRH30-Rhabdomyosarcoma | 1.6 |
| carcinoma | | (met to bone marrow) | |
| NCI-SNU-1-Gastric | 6.0 | A431-Epidermoid carcinoma | 0.0 |
| carcinoma | | | |
| RF-1-Gastric | 0.1 | WM266-4-Melanoma | 7.0 |
| adenocarcinoma | | | |
| RF-48-Gastric | 0.5 | DU 145-Prostate carcinoma | 0.0 |
| adenocarcinoma | | (brain metastasis) | |
| MKN-45-Gastric | 7.2 | MDA-MB-468-Breast | 0.3 |
| carcinoma | | adenocarcinoma | |
| NCI-N87-Gastric | 6.7 | SCC-4-Squamous cell | 0.0 |
| carcinoma | | carcinoma of tongue | |
| OVCAR-5-Ovarian | 1.1 | SCC-9-Squamous cell | 0.0 |
| carcinoma | | carcinoma of tongue | |
| RL95-2-Uterine | 0.6 | SCC-15-Squamous cell | 0.1 |
| carcinoma | | carcinoma of tongue | |
| HelaS3-Cervical | 0.3 | CAL 27-Squamous cell | 0.5 |
| adenocarcinoma | | carcinoma of tongue | |

TABLE TJ
Panel 4D

| | Rel. | Rel. | Rel. | | Rel. | Rel. | Rel. |
|---|---|---|---|---|---|---|---|
| | Exp.(%) | Exp.(%) | Exp.(%) | | Exp.(%) | Exp.(%) | Exp.(%) |
| | Ag2763, | Ag92, | Ag92b, | | Ag2763, | Ag92, | Ag92b, |
| | Run | Run | Run | | Run | Run | Run |
| Tissue Name | 162015245 | 149912706 | 151965831 | Tissue Name | 162015245 | 149912706 | 151965831 |
| Secondary Th1 act | 0.0 | 0.0 | 0.1 | HUVEC IL- | 0.1 | 0.2 | 0.6 |
| | | | | 1 beta | | | |
| Secondary Th2 act | 0.0 | 0.0 | 0.2 | HUVEC IFN | 0.1 | 0.3 | 0.2 |
| | | | | gamma | | | |
| Secondary Tr1 act | 0.0 | 0.0 | 0.0 | HUVEC TNF | 0.1 | 0.1 | 0.1 |
| | | | | alpha + IFN | | | |
| | | | | gamma | | | |
| Secondary Th1 | 0.0 | 0.0 | 0.0 | HUVEC TNF | 1.4 | 0.9 | 1.1 |
| rest | | | | alpha + IL4 | | | |
| Secondary Th2 | 0.0 | 0.0 | 0.0 | HUVEC IL-11 | 0.1 | 0.1 | 0.2 |
| rest | | | | | | | |
| Secondary Tr1 | 0.0 | 0.0 | 0.0 | Lung | 0.0 | 0.0 | 0.2 |
| rest | | | | Microvascular | | | |
| | | | | EC none | | | |
| Primary Th1 act | 0.0 | 0.0 | 0.0 | Lung | 0.0 | 0.0 | 0.1 |
| | | | | Microvascular | | | |
| | | | | EC TNF alpha + | | | |
| | | | | IL-1 beta | | | |
| Primary Th2 act | 0.0 | 0.0 | 0.0 | Microvascular | 0.0 | 0.0 | 0.1 |
| | | | | Dermal EC none | | | |
| Primary Tr1 act | 0.0 | 0.0 | 0.1 | Microsvasular | 0.0 | 0.0 | 0.0 |
| | | | | Dermal EC | | | |
| | | | | TNF alpha + IL- | | | |
| | | | | 1 beta | | | |
| Primary Th1 rest | 0.0 | 0.0 | 0.0 | Bronchial | 34.9 | 15.0 | 4.0 |
| | | | | epithelium | | | |
| | | | | TNF alpha + | | | |
| | | | | IL1 beta | | | |
| Primary Th2 rest | 0.0 | 0.0 | 0.0 | Small airway | 11.5 | 11.2 | 10.1 |
| | | | | epithelium none | | | |
| Primary Tr1 rest | 0.0 | 0.0 | 0.0 | Small airway | 77.9 | 81.2 | 87.1 |
| | | | | epithelium | | | |
| | | | | TNF alpha + IL- | | | |
| | | | | 1 beta | | | |
| CD45RA CD4 | 0.6 | 1.4 | 1.3 | Coronery artery | 0.2 | 0.2 | 0.3 |
| lymphocyte act | | | | SMC rest | | | |
| CD45RO CD4 | 0.0 | 0.0 | 0.0 | Coronery artery | 0.1 | 0.1 | 0.2 |
| lymphocyte act | | | | SMC TNF alpha + | | | |
| | | | | IL-1 beta | | | |
| CD8 lymphocyte | 0.0 | 0.0 | 0.0 | Astrocytes rest | 2.6 | 3.1 | 2.8 |
| act | | | | | | | |
| Secondary CD8 | 0.0 | 0.0 | 0.0 | Astrocytes | 1.1 | 1.2 | 1.4 |
| lymphocyte rest | | | | TNF alpha + IL- | | | |
| | | | | 1 beta | | | |
| Secondary CD8 | 0.0 | 0.0 | 0.0 | KU-812 | 1.0 | 1.5 | 1.3 |
| lymphocyte act | | | | (Basophil) rest | | | |
| CD4 lymphocyte | 0.0 | 0.0 | 0.0 | KU-812 | 2.1 | 1.4 | 1.1 |
| none | | | | (Basophil) | | | |
| | | | | PMA/ionomycin | | | |
| 2ry | 0.0 | 0.0 | 0.0 | CCD1106 | 100.0 | 81.2 | 60.3 |
| Th1/Th2/Tr1_anti- | | | | (Keratinocytes) | | | |
| CD95 CH11 | | | | none | | | |
| LAK cells rest | 0.0 | 0.0 | 0.0 | CCD1106 | 14.7 | 5.1 | 2.9 |
| | | | | (Keratinocytes) | | | |
| | | | | TNF alpha + IL- | | | |
| | | | | 1 beta | | | |
| LAK cells IL-2 | 0.0 | 0.0 | 0.0 | Liver cirrhosis | 4.6 | 5.1 | 4.9 |
| LAK cells IL- | 0.0 | 0.0 | 0.0 | Lupus kidney | 5.9 | 6.3 | 8.0 |
| 2 + IL-12 | | | | | | | |
| LAK cells IL- | 0.0 | 0.0 | 0.0 | NCI-H292 none | 2.1 | 2.0 | 1.4 |
| 2 + IFN gamma | | | | | | | |
| LAK cells IL-2 + | 0.1 | 0.0 | 0.0 | NCI-H292 IL-4 | 1.9 | 1.4 | 1.3 |
| IL-18 | | | | | | | |
| LAK cells | 0.0 | 0.0 | 0.0 | NCI-H292 IL-9 | 1.7 | 3.4 | 1.5 |
| PMA/ionomycin | | | | | | | |
| NK Cells IL-2 rest | 0.0 | 0.0 | 0.0 | NCI-H292 IL- | 0.9 | 1.8 | 0.8 |
| | | | | 13 | | | |
| Two Way MLR 3 | 0.0 | 0.0 | 0.0 | NCI-H292 IFN | 0.5 | 0.4 | 0.6 |
| day | | | | gamma | | | |
| Two Way MLR 5 | 0.0 | 0.0 | 0.0 | HPAEC none | 0.4 | 0.4 | 1.1 |
| day | | | | | | | |
| Two Way MLR 7 | 0.0 | 0.0 | 0.0 | HPAEC TNF | 0.0 | 0.0 | 0.3 |
| day | | | | alpha + IL-1 | | | |
| | | | | beta | | | |
| PBMC rest | 0.0 | 0.0 | 0.0 | Lung fibroblast | 6.9 | 9.6 | 9.2 |
| | | | | none | | | |
| PBMC PWM | 0.0 | 0.0 | 0.2 | Lung fibroblast | 11.8 | 4.6 | 15.5 |
| | | | | TNF alpha + IL- | | | |
| | | | | 1 beta | | | |
| PBMC PHA-L | 0.1 | 0.0 | 0.0 | Lung fibroblast | 37.9 | 42.3 | 47.3 |
| | | | | IL-4 | | | |
| Ramos (B cell) | 0.0 | 0.0 | 0.2 | Lung fibroblast | 18.8 | 18.3 | 23.3 |
| none | | | | IL-9 | | | |
| Ramos (B cell) | 0.0 | 0.0 | 0.0 | Lung fibroblast | 17.3 | 20.0 | 23.8 |
| ionomycin | | | | IL-13 | | | |
| B lymphocytes | 0.0 | 0.0 | 0.2 | Lung fibroblast | 14.1 | 11.8 | 12.7 |
| PWM | | | | IFN gamma | | | |
| B lymphocytes | 0.1 | 0.1 | 0.1 | Dermal | 4.9 | 4.4 | 4.4 |
| CD40L and IL-4 | | | | fibroblast | | | |
| | | | | CCD1070 rest | | | |
| EOL-1 dbcAMP | 0.0 | 0.0 | 0.0 | Dermal | 1.3 | 1.9 | 2.9 |
| | | | | fibroblast | | | |
| | | | | CCD1070 TNF | | | |
| | | | | alpha | | | |
| EOL-1 dbcAMP | 0.0 | 0.0 | 0.0 | Dermal | 0.7 | 1.5 | 1.6 |
| PMA/ionomycin | | | | fibroblast | | | |
| | | | | CCD1070 IL-1 | | | |
| | | | | beta | | | |
| Dendritic cells | 0.0 | 0.0 | 0.0 | Dermal | 0.3 | 0.2 | 0.7 |
| none | | | | fibroblast IFN | | | |
| | | | | gamma | | | |
| Dendritic cells | 0.0 | 0.1 | 0.1 | Dermal | 3.6 | 6.6 | 6.6 |
| LPS | | | | fibroblast IL-4 | | | |
| Dendritic cells | 0.0 | 0.0 | 0.0 | IBD Colitis 2 | 0.4 | 0.2 | 0.0 |
| anti-CD40 | | | | | | | |
| Monocytes rest | 0.0 | 0.0 | 0.0 | IBD Crohn's | 0.6 | 0.3 | 0.4 |
| Monocytes LPS | 0.0 | 0.0 | 0.0 | Colon | 5.5 | 6.5 | 4.1 |
| Macrophages rest | 0.0 | 0.0 | 0.1 | Lung | 58.6 | 57.0 | 69.3 |
| Macrophages LPS | 0.0 | 0.0 | 0.0 | Thymus | 84.7 | 100.0 | 100.0 |
| HUVEC none | 0.5 | 0.5 | 0.5 | Kidney | 13.5 | 15.1 | 25.0 |
| HUVEC starved | 1.0 | 0.4 | 0.5 | | | | |

AI_comprehensive panel_v1.0 Summary: Ag92 Highest expression of the CG50838-01 gene is found in in rheumatoid arthritis (RA) synovium fluid cells (CT=28). This gene shows higher expression in synovium, cartilage and bone samples from rheumatide arthritis patient (CTs=28-29) as compared to orthoarthritis patients (CTs=31-35). Therefore, therapeutic modulation of the activity of the GPCR encoded by this gene may be useful in the treatment of patients suffering from rheumatoid arthritis.

In addition, low expression of this gene is seen in almost all the samples derived from normal and diseased patients. Please see panel 4D for potential utility of this gene.

CNS_neurodegeneration_v1.0 Summary: Ag92b This panel does not show differential expression of the CG50838-01 gene in Alzheimer's disease. However, this expression profile confirms the presence of this gene in the brain. Please see Panel 1 and 1.3D for discussion of utility of this gene in the central nervous system. Results from two additional experiments with the probe and primer sets Ag92 and Ag2763 are not included. The amp plot indicates that there were experimental difficulties with these runs.

Panel 1 Summary: Ag92/Ag92b Three experiments show highest expression of the CG50838-01 gene in the cerebellum (CTs=24-27). In addition, expression is seen in the hippocampus, thalamus and hypothalamus. This gene encodes a fibronectin leucine rich transmembrane protein 3 (FLRT3), a member of the leucine rich repeats (LRR) protein family. FLRT proteins may play a role in cell adhesion and/or receptor signaling (Ref. 1). Several of LRR proteins, such as connectin, slit, chaoptin, and Toll have pivotal roles in neuronal development in Drosophila and may play significant but distinct roles in neural development and in the adult nervous system of humans (Ref. 2). In Drosophilia, the LRR region of axon guidance proteins has been shown to be critical for their function (especially in axon repulsion). Since the leucine-rich-repeat protein encoded by this gene shows high expression in the cerebellum, it is an excellent candidate neuronal guidance protein for axons, dendrites and/or growth cones in general. Therefore, therapeutic modulation of the levels of this protein, or possible signaling via this protein, may be of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease).

Among tissues with metabolic function, this gene is expressed at moderate to low levels in pituitary, adrenal gland, pancreas, thyroid, skeletal muscle, heart, and adult and fetal liver. This widespread expression among these tissues suggests that this gene product may play a role in normal neuroendocrine and metabolic and that disregulated expression of this gene may contribute to neuroendocrine disorders or metabolic diseases, such as obesity and diabetes.

In addition, there is expression in cell lines derived from pancreatic, gastric, colon, ovarian and lung cancer cell lines. Thus, based upon this pattern of gene expression, the therapeutic modulation of the activity of the this gene product might be of use in the treatment of these cancers.

Reference.

1. Lacy S E, Bonnemann C G, Buzney E A, Kunkel L M. Identification of FLRT1, FLRT2, and FLRT3: a novel family of transmembrane leucine-rich repeat proteins. Genomics Dec. 15, 1999; 15;62(3):417-26

2. Battye R., Stevens A., Perry R. L., Jacobs J. R. (2001) Repellent signaling by Slit requires the leucine-rich repeats. J. Neurosci. 21: 4290-4298.

Panel 1.3D Summary: Ag2763/Ag92b Two experiments with the different probe and primer sets produce results that are in excellent agreement, with high expression of the CG50838-01 gene in gastric cancer NCI-N87, colon cancer CoCa2, ovarian cancer OVCAR-5 (CTs=29-31). Thus, expression of this gene can be used as a marker for detection these cancers. In addition, therapeutic modulation of the activity of this gene or its protein product, through the use of small molecule drugs, protein therapeutics or antibodies, might be beneficial in the treatment of these cancers.

Among tissues with metabolic or endocrine function, this gene is expressed at high to moderate levels in adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at high levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease epilepsy, multiple sclerosis, schizophrenia and depression.

Panel 2D Summary: Ag2763/Ag92/Ag92b Three experiments with the different probe and primer sets produce results that are in excellent agreement, with highest expression of the CG50838-01 gene in the kidney cancer (OD04450-01) sample (CTs=27). In addition, expression of this gene is much lower in the corresponding control margin sample (CT=29). Significant expression of this gene is also seen in nuclear grade 2 kidney cancer and breast cancers. Therefore, therapeutic modulation of the activity of this gene or its protein product, through the use of small molecule drugs, protein therapeutics or antibodies, might be beneficial in the treatment of kidney or breast cancer.

Panel 3D Summary: Ag92b Highest expression of the CG50838-01 gene is detected in a sample derived from a brain cancer cell line (XF-498) and NCI-H727-lung carcinoid (CTs=27). Therefore, expression of this gene can be used to distinguish these samples from other samples in this panel. In addition, substantial expression of this gene is also seen in primitive neuroectodermal, medulloblastoma, mucoepidermoid lung carcinoma, small cell lung cancer, gastric cancer, ovarian cancer, pancreatic cancer, osteosarcoma, fibrosarcoma and colon cancer cell lines. Therefore, therapeutic modulation of the activity of this gene or its protein product, through the use of small molecule drugs, protein therapeutics or antibodies, might be beneficial in the treatment of these cancers.

In addition this panel also confirms expression of this gene in CNS, especially cerebellum. See panel 1 for potential utility of this gene.

Panel 4D Summary: Ag2763, 92a, 92b Three experiments with the different probe and primer sets produce results that are in excellent agreement, with high expression of this gene in untreated CCD1106 (keratinocytes), thymus, TNFalpha+IL-1beta treated small airway epithelium, and lung (CTs=27-28). Therefore, expression of this gene can be used to distinguish this sample from other samples in the panel. Interestingly, expression of this gene considerably reduced in untreated small airway epithelium and TNFalpha+IL-1beta treated keratinocytes. Thus, expression of this gene can be used to distinguish between these treated versus untreated cells.

In addition, significant expression of this gene is also seen in activated CD45RA CD4 lymphocyte, TNFalpha+IL1beta treated bronchial epithelium, NCI-H292 cells, lung and dermal fibroblasts, liver cirrhosis, lupus kidney, lung and kidney. This gene codes for a fibronectin leucine rich transmembrane protein 3 (FLRT3) like molecule, a member of the fibronectin leucine rich transmembrane protein (FLRT) family. These proteins may play a role in cell adhesion and/or receptor signaling (See Ref. 1 in panel 1). This gene may play a role in maintaining normal integrity of lung, and kidney tissue. Therefore, therapeutic modulation of the activity of the FLRT3 encoded by this gene may be beneficial for the reduction or elimination of the symptoms caused by inflammation in lung epithelia in chronic obstructive pulmonary disease, and in asthma, allergy, emphysema, kidney related diseases such as lupus and glomerulonephritis and liver cirrhosis.

### U. NOV28a (CG58567-01: PROTOCADHERIN)

Expression of gene CG58567-01 was assessed using the primer-probe set Ag2897, described in Table UA. Results of the RTQ-PCR runs are shown in Tables UB, UC, UD, UE, UF, UG and UH.

TABLE UA
Probe Name Ag2897

| | | | | Start |
|---|---|---|---|---|
| Primers | Sequences | | Length | Position |
| Forward | 5′-tgacaggggatattcatgctaa-3′ | (SEQ ID NO:293) | 22 | 1565 |
| Probe | TET-5′-tggcaataaatactgcctcacagtcca-3′-TAMRA | (SEQ ID NO:294) | 27 | 1608 |
| Reverse | 5′-agttgcatcacctttgtctttg-3′ | (SEQ ID NO:295) | 22 | 1638 |

TABLE UB
AI_comprehensive panel_v1.0

| | Rel. Exp.(%) Ag2897, | | Rel. Exp.(%) Ag2897, |
|---|---|---|---|
| Tissue Name | Run 229662505 | Tissue Name | Run 229662505 |
| 110967 COPD-F | 4.7 | 112427 Match Control | 100.0 |
| | | Psoriasis-F | |
| 110980 COPD-F | 22.5 | 112418 Psoriasis-M | 2.8 |
| 110968 COPD-M | 8.5 | 112723 Match Control | 14.3 |
| | | Psoriasis-M | |
| 110977 COPD-M | 51.8 | 112419 Psoriasis-M | 10.7 |
| 110989 Emphysema-F | 51.8 | 112424 Match Control | 8.7 |
| | | Psoriasis-M | |
| 110992 Emphysema-F | 5.0 | 112420 Psoriasis-M | 40.1 |
| 110993 Emphysema-F | 5.6 | 112425 Match Control | 65.1 |
| | | Psoriasis-M | |
| 110994 Emphysema-F | 3.3 | 104689 (MF) OA Bone- | 1.7 |
| | | Backus | |
| 110995 Emphysema-F | 13.2 | 104690 (MF) Adj | 4.5 |
| | | “Normal” Bone-Backus | |
| 110996 Emphysema-F | 0.6 | 104691 (MF) OA | 9.5 |
| | | Synovium-Backus | |
| 110997 Asthma-M | 1.4 | 104692 (BA) OA | 3.1 |
| | | Cartilage-Backus | |
| 111001 Asthma-F | 8.1 | 104694 (BA) OA Bone- | 1.3 |
| | | Backus | |
| 111002 Asthma-F | 12.5 | 104695 (BA) Adj | 1.8 |
| | | “Normal” Bone-Backus | |
| 111003 Atopic | 12.8 | 104696 (BA) OA | 5.0 |
| Asthma-F | | Synovium-Backus | |
| 111004 Atopic | 12.3 | 104700 (SS) OA Bone- | 5.2 |
| Asthma-F | | Backus | |
| 111005 Atopic | 6.1 | 104701 (SS) Adj | 7.0 |
| Asthma-F | | “Normal” Bone-Backus | |
| 111006 Atopic | 3.7 | 104702 (SS) OA | 12.7 |
| Asthma-F | | Synovium-Backus | |
| 111417 Allergy-M | 7.7 | 117093 OA Cartilage | 15.1 |
| | | Rep7 | |
| 112347 Allergy-M | 0.8 | 112672 OA Bone5 | 15.9 |
| 112349 Normal | 0.8 | 112673 OA Synovium5 | 8.2 |
| Lung-F | | | |
| 112357 Normal | 20.2 | 112674 OA Synovial | 7.9 |
| Lung-F | | Fluid cells5 | |
| 112354 Normal | 2.1 | 117100 OA Cartilage | 1.3 |
| Lung-M | | Rep14 | |
| 112374 Crohns-F | 4.7 | 112756 OA Bone9 | 0.8 |
| 112389 Match Control | 2.1 | 112757 OA Synovium9 | 0.4 |
| Crohns-F | | | |
| 112375 Crohns-F | 6.7 | 112758 OA Synovial | 10.2 |
| | | Fluid Cells9 | |
| 112732 Match Control | 2.2 | 117125 RA Cartilage | 6.5 |
| Crohns-F | | Rep2 | |
| 112725 Crohns-M | 18.9 | 113492 Bone2 RA | 2.2 |
| 112387 Match Control | 11.2 | 113493 Synovium2 RA | 0.2 |
| Crohns-M | | | |
| 112378 Crohns-M | 1.3 | 113494 Syn Fluid Cells | 2.0 |
| | | RA | |
| 112390 Match Control | 62.4 | 113499 Cartilage4 RA | 1.6 |
| Crohns-M | | | |
| 112726 Crohns-M | 9.6 | 113500 Bone4 RA | 1.3 |
| 112731 Match Control | 16.0 | 113501 Synovium4 RA | 1.9 |
| Crohns-M | | | |
| 112380 Ulcer Col-F | 21.2 | 113502 Syn Fluid | 1.0 |
| | | Cells4 RA | |
| 112734 Match Control | 4.5 | 113495 Cartilage3 RA | 1.1 |
| Ulcer Col-F | | | |
| 112384 Ulcer Col-F | 17.3 | 113496 Bone3 RA | 1.4 |
| 112737 Match Control | 5.2 | 113497 Synovium3 RA | 0.2 |
| Ulcer Col-F | | | |
| 112386 Ulcer Col-F | 5.3 | 113498 Syn Fluid | 1.2 |
| | | Cells3 RA | |
| 112738 Match Control | 2.7 | 117106 Normal | 0.3 |
| Ulcer Col-F | | Cartilage Rep20 | |
| 112381 Ulcer Col-M | 0.3 | 113663 Bone3 Normal | 2.8 |
| 112735 Match Control | 23.7 | 113664 Synovium3 | 0.2 |
| Ulcer Col-M | | Normal | |
| 112382 Ulcer Col-M | 5.8 | 113665 Syn Fluid | 1.3 |
| | | Cells3 Normal | |
| 112394 Match Control | 6.5 | 117107 Normal | 9.2 |
| Ulcer Col-M | | Cartilage Rep22 | |
| 112383 Ulcer Col-M | 1.6 | 113667 Bone4 Normal | 9.5 |
| 112736 Match Control | 1.3 | 113668 Synovium4 | 6.1 |
| Ulcer Col-M | | Normal | |
| 112423 Psoriasis-F | 7.9 | 113669 Syn Fluid | 10.2 |
| | | Cells4 Normal | |

TABLE UC
CNS_neurodegeneration_v1.0

| | Rel. Exp.(%) Ag2897, | | Rel. Exp.(%) Ag2897, |
|---|---|---|---|
| Tissue Name | Run 209734745 | Tissue Name | Run 209734745 |
| AD 1 Hippo | 6.5 | Control (Path) 3 | 8.9 |
| | | Temporal Ctx | |
| AD 2 Hippo | 31.9 | Control (Path) 4 | 46.7 |
| | | Temporal Ctx | |
| AD 3 Hippo | 1.6 | AD 1 Occipital Ctx | 5.6 |
| AD 4 Hippo | 7.3 | AD 2 Occipital Ctx | 0.0 |
| | | (Missing) | |
| AD 5 hippo | 46.3 | AD 3 Occipital Ctx | 6.1 |
| AD 6 Hippo | 44.4 | AD 4 Occipital Ctx | 23.0 |
| Control 2 Hippo | 24.0 | AD 5 Occipital Ctx | 11.3 |
| Control 4 Hippo | 15.7 | AD 6 Occipital Ctx | 22.5 |
| Control (Path) 3 | 0.0 | Control 1 Occipital | 1.6 |
| Hippo | | Ctx | |
| AD 1 Temporal Ctx | 3.0 | Control 2 Occipital | 17.7 |
| | | Ctx | |
| AD 2 Temporal Ctx | 38.7 | Control 3 Occipital | 9.1 |
| | | Ctx | |
| AD 3 Temporal Ctx | 3.5 | Control 4 Occipital | 8.7 |
| | | Ctx | |
| AD 4 Temporal Ctx | 23.0 | Control (Path) 1 | 92.0 |
| | | Occipital Ctx | |
| AD 5 Inf Temporal | 79.0 | Control (Path) 2 | 0.8 |
| Ctx | | Occipital Ctx | |
| AD 5 Sup Temporal | 43.5 | Control (Path) 3 | 1.6 |
| Ctx | | Occipital Ctx | |
| AD 6 Inf Temporal | 41.8 | Control (Path) 4 | 7.6 |
| Ctx | | Occipital Ctx | |
| AD 6 Sup Temporal | 41.2 | Control 1 Parietal | 4.2 |
| Ctx | | Ctx | |
| Control 1 Temporal | 12.3 | Control 2 Parietal | 47.3 |
| Ctx | | Ctx | |
| Control 2 Temporal | 50.3 | Control 3 Parietal | 10.1 |
| Ctx | | Ctx | |
| Control 3 Temporal | 17.8 | Control (Path) 1 | 100.0 |
| Ctx | | Parietal Ctx | |
| Control 4 Temporal | 9.3 | Control (Path) 2 | 13.4 |
| Ctx | | Parietal Ctx | |
| Control (Path) 1 | 70.2 | Control (Path) 3 | 4.2 |
| Temporal Ctx | | Parietal Ctx | |
| Control (Path) 2 | 29.3 | Control (Path) 4 | 33.2 |
| Temporal Ctx | | Parietal Ctx | |

TABLE UD
Panel 1.3D

| | Rel. Exp.(%) | Rel. Exp.(%) | | Rel. Exp.(%) | Rel. Exp.(%) |
|---|---|---|---|---|---|
| | Ag2897, Run | Ag2897, Run | | Ag2897, Run | Ag2897, Run |
| Tissue Name | 161905860 | 165518166 | Tissue Name | 161905860 | 165518166 |
| Liver | 0.0 | 0.0 | Kidney (fetal) | 2.8 | 2.4 |
| adenocarcinoma | | | | | |
| Pancreas | 1.4 | 2.4 | Renal ca. 786-0 | 0.9 | 3.3 |
| Pancreatic ca. | 0.0 | 0.0 | Renal ca. A498 | 13.9 | 23.7 |
| CAPAN 2 | | | | | |
| Adrenal gland | 1.4 | 0.0 | Renal ca. RXF | 0.0 | 0.0 |
| | | | 393 | | |
| Thyroid | 0.0 | 0.0 | Renal ca. | 0.0 | 0.0 |
| | | | ACHN | | |
| Salivary gland | 0.0 | 0.0 | Renal ca. UO- | 0.0 | 0.0 |
| | | | 31 | | |
| Pituitary gland | 4.1 | 9.0 | Renal ca. TK- | 0.0 | 0.0 |
| | | | 10 | | |
| Brain (fetal) | 7.1 | 17.8 | Liver | 0.0 | 2.9 |
| Brain (whole) | 21.0 | 59.0 | Liver (fetal) | 0.4 | 0.0 |
| Brain (amygdala) | 24.7 | 37.9 | Liver ca. | 0.0 | 0.0 |
| | | | (hepatoblast) | | |
| | | | HepG2 | | |
| Brain (cerebellum) | 5.6 | 28.5 | Lung | 1.4 | 0.0 |
| Brain | 25.2 | 25.3 | Lung (fetal) | 6.8 | 11.0 |
| (hippocampus) | | | | | |
| Brain (substantia | 0.9 | 17.1 | Lung ca. | 0.0 | 0.0 |
| nigra) | | | (small cell) | | |
| | | | LX-1 | | |
| Brain (thalamus) | 4.1 | 23.0 | Lung ca. | 0.0 | 4.1 |
| | | | (small cell) | | |
| | | | NCI-H69 | | |
| Cerebral Cortex | 100.0 | 22.2 | Lung ca. (s.cell | 1.0 | 0.0 |
| | | | var.) SHP-77 | | |
| Spinal cord | 68.8 | 100.0 | Lung ca. (large | 0.0 | 0.0 |
| | | | cell) NCI-H460 | | |
| glio/astro U87-MG | 54.3 | 10.4 | Lung ca. (non- | 0.0 | 0.0 |
| | | | sm. cell) A549 | | |
| glio/astro U-118- | 1.5 | 20.4 | Lung ca. (non- | 0.0 | 3.0 |
| MG | | | s.cell) NCI- | | |
| | | | H23 | | |
| astrocytoma | 16.3 | 0.0 | Lung ca. (non- | 0.6 | 7.5 |
| SW1783 | | | s.cell) HOP-62 | | |
| neuro*; met SK-N- | 0.0 | 0.0 | Lung ca. (non- | 0.0 | 0.0 |
| AS | | | s.cl) NCI-H522 | | |
| astrocytoma SF- | 0.0 | 4.3 | Lung ca. | 4.6 | 8.6 |
| 539 | | | (squam.) SW | | |
| | | | 900 | | |
| astrocytoma SNB- | 18.4 | 28.7 | Lung ca. | 0.0 | 0.0 |
| 75 | | | (squam.) NCI- | | |
| | | | H596 | | |
| glioma SNB-19 | 0.0 | 2.6 | Mammary | 7.9 | 17.1 |
| | | | gland | | |
| glioma U251 | 3.1 | 27.0 | Breast ca.* | 0.0 | 0.0 |
| | | | (pl.ef) MCF-7 | | |
| glioma SF-295 | 4.8 | 2.2 | Breast ca.* | 0.0 | 14.5 |
| | | | (pl.ef) MDA- | | |
| | | | MB-231 | | |
| Heart (fetal) | 0.0 | 0.0 | Breast ca.* | 0.0 | 0.0 |
| | | | (pl.ef) T47D | | |
| Heart | 1.4 | 0.0 | Breast ca. BT- | 0.0 | 12.2 |
| | | | 549 | | |
| Skeletal muscle | 77.4 | 0.0 | Breast ca. | 0.0 | 0.0 |
| (fetal) | | | MDA-N | | |
| Skeletal muscle | 1.1 | 14.1 | Ovary | 12.1 | 6.1 |
| Bone marrow | 0.0 | 2.5 | Ovarian ca. | 3.8 | 12.1 |
| | | | OVCAR-3 | | |
| Thymus | 2.1 | 2.4 | Ovarian ca. | 0.0 | 0.0 |
| | | | OVCAR-4 | | |
| Spleen | 0.0 | 4.4 | Ovarian ca. | 32.5 | 25.5 |
| | | | OVCAR-5 | | |
| Lymph node | 0.0 | 4.8 | Ovarian ca. | 1.9 | 0.0 |
| | | | OVCAR-8 | | |
| Colorectal | 10.7 | 0.0 | Ovarian ca. | 0.0 | 0.0 |
| | | | IGROV-1 | | |
| Stomach | 8.7 | 64.6 | Ovarian ca.* | 0.0 | 0.0 |
| | | | (ascites) SK- | | |
| | | | OV-3 | | |
| Small intestine | 7.2 | 18.9 | Uterus | 12.4 | 66.4 |
| Colon ca. SW480 | 0.0 | 0.0 | Placenta | 0.0 | 2.3 |
| Colon ca.* | 0.0 | 0.0 | Prostate | 0.0 | 0.0 |
| SW620(SW480 | | | | | |
| met) | | | | | |
| Colon ca. HT29 | 0.0 | 0.0 | Prostate ca.* | 0.0 | 0.0 |
| | | | (bone met)PC-3 | | |
| Colon ca. HCT- | 0.0 | 0.0 | Testis | 56.3 | 68.8 |
| 116 | | | | | |
| Colon ca. CaCo-2 | 0.0 | 0.0 | Melanoma | 1.3 | 8.2 |
| | | | Hs688(A).T | | |
| Colon ca. | 1.7 | 8.1 | Melanoma* | 2.5 | 4.8 |
| tissue(ODO3866) | | | (met) | | |
| | | | Hs688(B).T | | |
| Colon ca. HCC- | 0.0 | 0.0 | Melanoma | 1.5 | 2.8 |
| 2998 | | | UACC-62 | | |
| Gastric ca.* (liver | 10.3 | 7.7 | Melanoma | 0.0 | 0.0 |
| met) NCI-N87 | | | M14 | | |
| Bladder | 14.2 | 29.3 | Melanoma | 0.0 | 0.0 |
| | | | LOX IMVI | | |
| Trachea | 4.1 | 0.0 | Melanoma* | 0.0 | 0.0 |
| | | | (met) SK- | | |
| | | | MEL-5 | | |
| Kidney | 0.0 | 0.0 | Adipose | 7.2 | 0.0 |

TABLE UE
Panel 2D

| | Rel. Exp.(%) | Rel. Exp.(%) | | Rel. Exp.(%) | Rel. Exp.(%) |
|---|---|---|---|---|---|
| | Ag2897, Run | Ag2897, Run | | Ag2897, Run | Ag2897, Run |
| Tissue Name | 161905861 | 164997312 | Tissue Name | 161905861 | 164997312 |
| Normal Colon | 83.5 | 90.1 | Kidney Margin | 1.3 | 0.9 |
| | | | 8120608 | | |
| CC Well to Mod | 0.0 | 7.2 | Kidney Cancer | 1.4 | 1.6 |
| Diff (ODO3866) | | | 8120613 | | |
| CC Margin | 16.0 | 16.6 | Kidney Margin | 0.9 | 1.5 |
| (ODO3866) | | | 8120614 | | |
| CC Gr.2 | 0.0 | 0.0 | Kidney Cancer | 21.6 | 16.0 |
| rectosigmoid | | | 9010320 | | |
| (ODO3868) | | | | | |
| CC Margin | 6.3 | 3.6 | Kidney Margin | 1.9 | 0.5 |
| (ODO3868) | | | 9010321 | | |
| CC Mod Diff | 0.9 | 2.1 | Normal Uterus | 9.5 | 10.9 |
| (ODO3920) | | | | | |
| CC Margin | 19.1 | 11.7 | Uterus Cancer | 68.3 | 71.2 |
| (ODO3920) | | | 064011 | | |
| CC Gr.2 ascend | 8.8 | 9.2 | Normal | 3.3 | 0.8 |
| colon | | | Thyroid | | |
| (ODO3921) | | | | | |
| CC Margin | 6.9 | 7.5 | Thyroid | 2.0 | 6.9 |
| (ODO3921) | | | Cancer 064010 | | |
| CC from Partial | 0.9 | 3.3 | Thyroid | 8.9 | 12.2 |
| Hepatectomy | | | Cancer | | |
| (ODO4309) Mets | | | A302152 | | |
| Liver Margin | 0.7 | 2.0 | Thyroid | 1.0 | 0.7 |
| (ODO4309) | | | Margin | | |
| | | | A302153 | | |
| Colon mets to | 0.0 | 0.0 | Normal Breast | 15.9 | 15.3 |
| lung (OD04451- | | | | | |
| 01) | | | | | |
| Lung Margin | 1.9 | 1.0 | Breast Cancer | 4.3 | 6.3 |
| (OD04451-02) | | | (OD04566) | | |
| Normal Prostate | 3.9 | 10.7 | Breast Cancer | 7.3 | 2.8 |
| 6546-1 | | | (OD04590-01) | | |
| Prostate Cancer | 17.3 | 11.6 | Breast Cancer | 9.4 | 4.8 |
| (OD04410) | | | Mets | | |
| | | | (OD04590-03) | | |
| Prostate Margin | 25.0 | 15.5 | Breast Cancer | 3.6 | 0.9 |
| (OD04410) | | | Metastasis | | |
| | | | (OD04655-05) | | |
| Prostate Cancer | 28.3 | 11.7 | Breast Cancer | 8.4 | 11.6 |
| (OD04720-01) | | | 064006 | | |
| Prostate Margin | 29.9 | 20.4 | Breast Cancer | 2.6 | 4.3 |
| (OD04720-02) | | | 1024 | | |
| Normal Lung | 16.2 | 15.1 | Breast Cancer | 1.9 | 2.0 |
| 061010 | | | 9100266 | | |
| Lung Met to | 1.2 | 0.0 | Breast Margin | 7.4 | 5.4 |
| Muscle | | | 9100265 | | |
| (ODO4286) | | | | | |
| Muscle Margin | 8.2 | 1.6 | Breast Cancer | 20.0 | 13.6 |
| (ODO4286) | | | A209073 | | |
| Lung Malignant | 7.0 | 2.2 | Breast Margin | 5.0 | 12.0 |
| Cancer | | | A209073 | | |
| (OD03126) | | | | | |
| Lung Margin | 2.3 | 3.0 | Normal Liver | 0.9 | 1.0 |
| (OD03126) | | | | | |
| Lung Cancer | 8.2 | 3.1 | Liver Cancer | 0.0 | 0.0 |
| (OD04404) | | | 064003 | | |
| Lung Margin | 11.1 | 10.2 | Liver Cancer | 2.4 | 0.0 |
| (OD04404) | | | 1025 | | |
| Lung Cancer | 0.0 | 0.9 | Liver Cancer | 0.0 | 0.0 |
| (OD04565) | | | 1026 | | |
| Lung Margin | 1.9 | 5.0 | Liver Cancer | 0.0 | 1.0 |
| (OD04565) | | | 6004-T | | |
| Lung Cancer | 95.3 | 55.9 | Liver Tissue | 0.0 | 0.0 |
| (OD04237-01) | | | 6004-N | | |
| Lung Margin | 4.0 | 2.3 | Liver Cancer | 0.0 | 0.0 |
| (OD04237-02) | | | 6005-T | | |
| Ocular Mel Met | 0.0 | 0.0 | Liver Tissue | 0.0 | 0.0 |
| to Liver | | | 6005-N | | |
| (ODO4310) | | | | | |
| Liver Margin | 2.7 | 0.8 | Normal | 18.0 | 13.8 |
| (ODO4310) | | | Bladder | | |
| Melanoma Mets | 0.0 | 1.7 | Bladder | 1.0 | 0.7 |
| to Lung | | | Cancer 1023 | | |
| (OD04321) | | | | | |
| Lung Margin | 10.1 | 6.6 | Bladder | 10.9 | 2.6 |
| (OD04321) | | | Cancer | | |
| | | | A302173 | | |
| Normal Kidney | 14.8 | 16.4 | Bladder | 0.0 | 1.9 |
| | | | Cancer | | |
| | | | (OD04718-01) | | |
| Kidney Ca, | 93.3 | 100.0 | Bladder | 33.2 | 31.0 |
| Nuclear grade 2 | | | Normal | | |
| (OD04338) | | | Adjacent | | |
| | | | (OD04718-03) | | |
| Kidney Margin | 14.3 | 2.0 | Normal Ovary | 0.0 | 4.8 |
| (OD04338) | | | | | |
| Kidney Ca | 5.0 | 2.0 | Ovarian | 100.0 | 62.0 |
| Nuclear grade 1/2 | | | Cancer 064008 | | |
| (OD04339) | | | | | |
| Kidney Margin | 4.1 | 3.0 | Ovarian | 49.7 | 38.7 |
| (OD04339) | | | Cancer | | |
| | | | (OD04768-07) | | |
| Kidney Ca, Clear | 27.0 | 17.4 | Ovary Margin | 6.3 | 2.5 |
| cell type | | | (OD04768-08) | | |
| (OD04340) | | | | | |
| Kidney Margin | 6.4 | 12.7 | Normal | 28.1 | 18.7 |
| (OD04340) | | | Stomach | | |
| Kidney Ca, | 1.9 | 2.1 | Gastric Cancer | 11.0 | 14.1 |
| Nuclear grade 3 | | | 9060358 | | |
| (OD04348) | | | | | |
| Kidney Margin | 4.7 | 6.2 | Stomach | 15.9 | 29.7 |
| (OD04348) | | | Margin | | |
| | | | 9060359 | | |
| Kidney Cancer | 50.0 | 38.4 | Gastric Cancer | 15.0 | 6.8 |
| (OD04622-01) | | | 9060395 | | |
| Kidney Margin | 4.2 | 0.0 | Stomach | 26.6 | 26.4 |
| (OD04622-03) | | | Margin | | |
| | | | 9060394 | | |
| Kidney Cancer | 2.6 | 3.6 | Gastric Cancer | 2.7 | 1.7 |
| (OD04450-01) | | | 9060397 | | |
| Kidney Margin | 14.2 | 10.5 | Stomach | 28.1 | 33.0 |
| (OD04450-03) | | | Margin | | |
| | | | 9060396 | | |
| Kidney Cancer | 1.9 | 0.0 | Gastric Cancer | 27.7 | 33.4 |
| 8120607 | | | 064005 | | |

TABLE UF
Panel 3D

| | Rel. Exp.(%) | | Rel. Exp.(%) |
|---|---|---|---|
| | Ag2897, Run | | Ag2897, Run |
| Tissue Name | 164629841 | Tissue Name | 164629841 |
| Daoy-Medulloblastoma | 0.0 | Ca Ski-Cervical epidermoid | 0.0 |
| | | carcinoma (metastasis) | |
| TE671-Medulloblastoma | 0.0 | ES-2-Ovarian clear cell | 0.8 |
| | | carcinoma | |
| D283 Med- | 0.0 | Ramos-Stimulated with | 0.0 |
| Medulloblastoma | | PMA/ionomycin 6h | |
| PFSK-1-Primitive | 0.0 | Ramos-Stimulated with | 0.0 |
| Neuroectodermal | | PMA/ionomycin 14h | |
| XF-498-CNS | 0.6 | MEG-01-Chronic | 0.0 |
| | | myelogenous leukemia | |
| | | (megokaryoblast) | |
| SNB-78-Glioma | 6.0 | Raji-Burkitt's lymphoma | 0.0 |
| SF-268-Glioblastoma | 0.0 | Daudi-Burkitt's lymphoma | 0.0 |
| T98G-Glioblastoma | 0.0 | U266-B-cell plasmacytoma | 0.0 |
| SK-N-SH- | 0.0 | CA46-Burkitt's lymphoma | 0.0 |
| Neuroblastoma | | | |
| (metastasis) | | | |
| SF-295-Glioblastoma | 0.0 | RL-non-Hodgkin's B-cell | 0.0 |
| | | lymphoma | |
| Cerebellum | 7.1 | JM1-pre-B-cell lymphoma | 0.0 |
| Cerebellum | 0.0 | Jurkat-T cell leukemia | 0.0 |
| NCI-H292- | 0.0 | TF-1-Erythroleukemia | 0.0 |
| Mucoepidermoid lung | | | |
| carcinoma | | | |
| DMS-114-Small cell | 0.0 | HUT 78-T-cell lymphoma | 0.0 |
| lung cancer | | | |
| DMS-79-Small cell lung | 0.8 | U937-Histiocytic lymphoma | 0.0 |
| cancer | | | |
| NCI-H146-Small cell | 1.3 | KU-812-Myelogenous | 0.0 |
| lung cancer | | leukemia | |
| NCI-H526-Small cell | 100.0 | 769-P-Clear cell renal | 0.0 |
| lung cancer | | carcinoma | |
| NCI-N417-Small cell | 0.0 | Caki-2-Clear cell renal | 0.9 |
| lung cancer | | carcinoma | |
| NCI-H82-Small cell lung | 0.0 | SW 839-Clear cell renal | 0.0 |
| cancer | | carcinoma | |
| NCI-H157-Squamous | 0.0 | G401-Wilms'tumor | 0.0 |
| cell lung cancer | | | |
| (metastasis) | | | |
| NCI-H1155-Large cell | 3.2 | Hs766T-Pancreatic carcinoma | 0.0 |
| lung cancer | | (LN metastasis) | |
| NCI-H1299-Large cell | 1.9 | CAPAN-1-Pancreatic | 0.0 |
| lung cancer | | adenocarcinoma (liver | |
| | | metastasis) | |
| NCI-H727-Lung | 0.0 | SU86.86-Pancreatic | 0.6 |
| carcinoid | | carcinoma (liver metastasis) | |
| NCI-UMC-11-Lung | 2.1 | BxPC-3-Pancreatic | 0.0 |
| carcinoid | | adenocarcinoma | |
| LX-1-Small cell lung | 0.0 | HPAC-Pancreatic | 0.0 |
| cancer | | adenocarcinoma | |
| Colo-205-Colon cancer | 0.0 | MIA PaCa-2-Pancreatic | 0.0 |
| | | carcinoma | |
| KM12-Colon cancer | 2.6 | CFPAC-1-Pancreatic ductal | 0.4 |
| | | adenocarcinoma | |
| KM20L2-Colon cancer | 0.0 | PANC-1-Pancreatic | 0.0 |
| | | epithelioid ductal carcinoma | |
| NCI-H716-Colon cancer | 31.0 | T24-Bladder carcinma | 0.0 |
| | | (transitional cell) | |
| SW-48-Colon | 0.0 | 5637-Bladder carcinoma | 0.0 |
| adenocarcinoma | | | |
| SW1116-Colon | 0.0 | HT-1197-Bladder carcinoma | 0.0 |
| adenocarcinoma | | | |
| LS 174T-Colon | 0.0 | UM-UC-3-Bladder carcinma | 0.0 |
| adenocarcinoma | | (transitional cell) | |
| SW-948-Colon | 0.0 | A204-Rhabdomyosarcoma | 0.0 |
| adenocarcinoma | | | |
| SW-480-Colon | 1.5 | HT-1080-Fibrosarcoma | 0.8 |
| adenocarcinoma | | | |
| NCI-SNU-5-Gastric | 0.0 | MG-63-Osteosarcoma | 0.0 |
| carcinoma | | | |
| KATO III-Gastric | 0.0 | SK-LMS-1-Leiomyosarcoma | 0.0 |
| carcinoma | | (vulva) | |
| NCI-SNU-16-Gastric | 0.8 | SJRH30-Rhabdomyosarcoma | 0.0 |
| carcinoma | | (met to bone marrow) | |
| NCI-SNU-1-Gastric | 0.0 | A431-Epidermoid carcinoma | 0.0 |
| carcinoma | | | |
| RF-1-Gastric | 0.0 | WM266-4-Melanoma | 1.2 |
| adenocarcinoma | | | |
| RF-48-Gastric | 0.8 | DU 145-Prostate carcinoma | 0.0 |
| adenocarcinoma | | (brain metastasis) | |
| MKN-45-Gastric | 0.0 | MDA-MB-468-Breast | 0.8 |
| carcinoma | | adenocarcinoma | |
| NCI-N87-Gastric | 0.3 | SCC-4-Squamous cell | 0.0 |
| carcinoma | | carcinoma of tongue | |
| OVCAR-5-Ovarian | 0.0 | SCC-9-Squamous cell | 0.0 |
| carcinoma | | carcinoma of tongue | |
| RL95-2-Uterine | 0.0 | SCC-15-Squamous cell | 0.0 |
| carcinoma | | carcinoma of tongue | |
| HelaS3-Cervical | 0.0 | CAL 27-Squamous cell | 0.0 |
| adenocarcinoma | | carcinoma of tongue | |

TABLE UG
Panel 4D

| | Rel. | Rel. | | Rel. | Rel. |
|---|---|---|---|---|---|
| | Exp.(%) | Exp.(%) | | Exp.(%) | Exp.(%) |
| | Ag2897, | Ag2897, | | Ag2897, | Ag2897, |
| | Run | Run | | Run | Run |
| Tissue Name | 161905862 | 163586314 | Tissue Name | 161905862 | 163586314 |
| Secondary Th1 act | 0.0 | 0.0 | HUVEC IL-1 beta | 0.0 | 0.0 |
| Secondary Th2 act | 0.0 | 0.0 | HUVEC IFN | 0.0 | 0.0 |
| | | | gamma | | |
| Secondary Tr1 act | 0.0 | 0.0 | HUVEC TNF | 0.0 | 0.0 |
| | | | alpha + IFN | | |
| | | | gamma | | |
| Secondary Th1 rest | 0.0 | 0.0 | HUVEC TNF | 0.0 | 0.0 |
| | | | alpha + IL4 | | |
| Secondary Th2 rest | 0.0 | 0.0 | HUVEC IL-11 | 0.0 | 0.0 |
| Secondary Tr1 rest | 0.0 | 0.0 | Lung | 0.0 | 0.0 |
| | | | Microvascular EC | | |
| | | | none | | |
| Primary Th1 act | 0.0 | 0.0 | Lung | 7.9 | 22.8 |
| | | | Microvascular EC | | |
| | | | TNF alpha + IL- | | |
| | | | 1 beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 16.2 | 0.0 |
| | | | Dermal EC | | |
| | | | TNF alpha + IL- | | |
| | | | 1 beta | | |
| Primary Th1 rest | 9.3 | 20.7 | Bronchial | 4.4 | 0.0 |
| | | | epithelium | | |
| | | | TNF alpha + | | |
| | | | IL1 beta | | |
| Primary Th2 rest | 0.0 | 9.0 | Small airway | 10.4 | 9.7 |
| | | | epithelium none | | |
| Primary Tr1 rest | 9.8 | 0.0 | Small airway | 100.0 | 75.8 |
| | | | epithelium | | |
| | | | TNF alpha + IL- | | |
| | | | 1 beta | | |
| CD45RA CD4 | 0.0 | 0.0 | Coronery artery | 0.0 | 9.9 |
| lymphocyte act | | | SMC rest | | |
| CD45RO CD4 | 0.0 | 0.0 | Coronery artery | 16.8 | 9.0 |
| lymphocyte act | | | SMC TNF alpha + | | |
| | | | IL-1 beta | | |
| CD8 lymphocyte | 0.0 | 0.0 | Astrocytes rest | 40.6 | 0.0 |
| act | | | | | |
| Secondary CD8 | 0.0 | 0.0 | Astrocytes | 56.6 | 100.0 |
| lymphocyte rest | | | TNF alpha + IL- | | |
| | | | 1 beta | | |
| Secondary CD8 | 0.0 | 0.0 | KU-812 (Basophil) | 0.0 | 0.0 |
| lymphocyte act | | | rest | | |
| CD4 lymphocyte | 0.0 | 0.0 | KU-812 (Basophil) | 0.0 | 0.0 |
| none | | | PMA/ionomycin | | |
| 2ry | 0.0 | 0.0 | CCD1106 | 0.0 | 0.0 |
| Th1/Th2/Tr1_anti- | | | (Keratinocytes) | | |
| CD95 CH11 | | | none | | |
| LAK cells rest | 10.9 | 0.0 | CCD1106 | 0.0 | 0.0 |
| | | | (Keratinocytes) | | |
| | | | TNF alpha + IL- | | |
| | | | 1 beta | | |
| LAK cells IL-2 | 0.0 | 0.0 | Liver cirrhosis | 17.2 | 8.2 |
| LAK cells IL-2 + | 0.0 | 0.0 | Lupus kidney | 8.4 | 9.6 |
| IL-12 | | | | | |
| LAK cells IL- | 0.0 | 0.0 | NCI-H292 none | 0.0 | 0.0 |
| 2 + IFN gamma | | | | | |
| LAK cells IL-2 + | 0.0 | 0.0 | NCI-H292 IL-4 | 0.0 | 0.0 |
| IL-18 | | | | | |
| LAK cells | 0.0 | 0.0 | NCI-H292 IL-9 | 0.0 | 0.0 |
| PMA/ionomycin | | | | | |
| NK Cells IL-2 rest | 0.0 | 0.0 | NCI-H292 IL-13 | 0.0 | 0.0 |
| Two Way MLR 3 | 0.0 | 9.3 | NCI-H292 IFN | 0.0 | 0.0 |
| day | | | gamma | | |
| Two Way MLR 5 | 0.0 | 0.0 | HPAEC none | 0.0 | 0.0 |
| day | | | | | |
| Two Way MLR 7 | 0.0 | 0.0 | HPAEC TNF | 0.0 | 0.0 |
| day | | | alpha + IL-1 beta | | |
| PBMC rest | 0.0 | 0.0 | Lung fibroblast | 0.0 | 0.0 |
| | | | none | | |
| PBMC PWM | 0.0 | 0.0 | Lung fibroblast | 0.0 | 18.8 |
| | | | TNF alpha + IL-1 | | |
| | | | beta | | |
| PBMC PHA-L | 8.9 | 0.0 | Lung fibroblast IL-4 | 0.0 | 17.7 |
| Ramos (B cell) | 0.0 | 0.0 | Lung fibroblast IL-9 | 0.0 | 0.0 |
| none | | | | | |
| Ramos (B cell) | 0.0 | 0.0 | Lung fibroblast IL- | 0.0 | 0.0 |
| ionomycin | | | 13 | | |
| B lymphocytes | 0.0 | 0.0 | Lung fibroblast | 0.0 | 0.0 |
| PWM | | | IFN gamma | | |
| B lymphocytes | 0.0 | 0.0 | Dermal fibroblast | 18.7 | 5.1 |
| CD40L and IL-4 | | | CCD1070 rest | | |
| EOL-1 dbcAMP | 0.0 | 0.0 | Dermal fibroblast | 45.4 | 29.5 |
| | | | CCD1070 TNF | | |
| | | | alpha | | |
| EOL-1 dbcAMP | 0.0 | 0.0 | Dermal fibroblast | 33.7 | 85.9 |
| PMA/ionomycin | | | CCD1070 IL-1 | | |
| | | | beta | | |
| Dendritic cells none | 0.0 | 0.0 | Dermal fibroblast | 0.0 | 0.0 |
| | | | IFN gamma | | |
| Dendritic cells LPS | 0.0 | 0.0 | Dermal fibroblast | 0.0 | 8.4 |
| | | | IL-4 | | |
| Dendritic cells anti- | 0.0 | 0.0 | IBD Colitis 2 | 0.0 | 9.9 |
| CD40 | | | | | |
| Monocytes rest | 0.0 | 0.0 | IBD Crohn's | 7.7 | 0.0 |
| Monocytes LPS | 0.0 | 0.0 | Colon | 62.4 | 39.2 |
| Macrophages rest | 0.0 | 0.0 | Lung | 9.9 | 22.1 |
| Macrophages LPS | 0.0 | 0.0 | Thymus | 27.7 | 35.8 |
| HUVEC none | 0.0 | 0.0 | Kidney | 18.9 | 20.6 |
| HUVEC starved | 0.0 | 0.0 | | | |

TABLE UH
Panel CNS_1

| | Rel. Exp.(%) | | Rel. Exp.(%) |
|---|---|---|---|
| | Ag2897, Run | | Ag2897, Run |
| Tissue Name | 171688457 | Tissue Name | 171688457 |
| BA4 Control | 29.7 | BA17 PSP | 24.3 |
| BA4 Control2 | 37.6 | BA17 PSP2 | 0.0 |
| BA4 | 0.0 | Sub Nigra Control | 25.9 |
| Alzheimer's2 | | | |
| BA4 Parkinson's | 23.8 | Sub Nigra Control2 | 33.4 |
| BA4 | 38.7 | Sub Nigra | 0.0 |
| Parkinson's2 | | Alzheimer's2 | |
| BA4 | 18.4 | Sub Nigra | 26.6 |
| Huntington's | | Parkinson's2 | |
| BA4 | 0.0 | Sub Nigra | 13.9 |
| Huntington's2 | | Huntington's | |
| BA4 PSP | 8.0 | Sub Nigra | 8.7 |
| | | Huntington's2 | |
| BA4 PSP2 | 37.6 | Sub Nigra PSP2 | 0.0 |
| BA4 Depression | 15.6 | Sub Nigra | 0.0 |
| | | Depression | |
| BA4 | 4.6 | Sub Nigra | 2.8 |
| Depression2 | | Depression2 | |
| BA7 Control | 56.6 | Glob Palladus | 4.1 |
| | | Control | |
| BA7 Control2 | 6.0 | Glob Palladus | 23.7 |
| | | Control2 | |
| BA7 | 7.3 | Glob Palladus | 3.8 |
| Alzheimer's2 | | Alzheimer's | |
| BA7 Parkinson's | 10.8 | Glob Palladus | 0.0 |
| | | Alzheimer's2 | |
| BA7 | 27.2 | Glob Palladus | 51.1 |
| Parkinson's2 | | Parkinson's | |
| BA7 | 56.3 | Glob Palladus | 3.5 |
| Huntington's | | Parkinson's2 | |
| BA7 | 10.8 | Glob Palladus PSP | 0.0 |
| Huntington's2 | | | |
| BA7 PSP | 21.5 | Glob Palladus PSP2 | 0.0 |
| BA7 PSP2 | 21.3 | Glob Palladus | 0.0 |
| | | Depression | |
| BA7 Depression | 5.4 | Temp Pole Control | 15.9 |
| BA9 Control | 31.6 | Temp Pole Control2 | 37.9 |
| BA9 Control2 | 100.0 | Temp Pole | 0.0 |
| | | Alzheimer's | |
| BA9 Alzheimer's | 3.8 | Temp Pole | 0.0 |
| | | Alzheimer's2 | |
| BA9 | 3.7 | Temp Pole | 34.2 |
| Alzheimer's2 | | Parkinson's | |
| BA9 Parkinson's | 19.2 | Temp Pole | 10.8 |
| | | Parkinson's2 | |
| BA9 | 30.6 | Temp Pole | 17.7 |
| Parkinson's2 | | Huntington's | |
| BA9 | 27.2 | Temp Pole PSP | 5.8 |
| Huntington's | | | |
| BA9 | 9.0 | Temp Pole PSP2 | 0.0 |
| Huntington's2 | | | |
| BA9 PSP | 0.0 | Temp Pole | 15.3 |
| | | Depression2 | |
| BA9 PSP2 | 16.5 | Cing Gyr Control | 76.3 |
| BA9 Depression | 2.7 | Cing Gyr Control2 | 54.0 |
| BA9 | 6.7 | Cing Gyr | 12.1 |
| Depression2 | | Alzheimer's | |
| BA17 Control | 25.9 | Cing Gyr | 2.9 |
| | | Alzheimer's2 | |
| BA17 Control2 | 14.8 | Cing Gyr | 26.6 |
| | | Parkinson's | |
| BA17 | 7.9 | Cing Gyr | 38.2 |
| Alzheimer's2 | | Parkinson's2 | |
| BA17 | 9.5 | Cing Gyr | 36.1 |
| Parkinson's | | Huntington's | |
| BA17 | 8.8 | Cing Gyr | 8.1 |
| Parkinson's2 | | Huntington's2 | |
| BA17 | 18.3 | Cing Gyr PSP | 8.3 |
| Huntington's | | | |
| BA17 | 0.0 | Cing Gry PSP2 | 4.0 |
| Huntington's2 | | | |
| BA17 | 7.6 | Cing Gyr | 0.0 |
| Depression | | Depression | |
| BA17 | 3.7 | Cing Gyr | 2.9 |
| Depression2 | | Depression2 | |

AI_comprehensive panel_v1.0 Summary: Ag2897 Highest expression of the CG58567-01 gene is detected in match control psoriasis sample (CT=30). Furthermore, this expression is down-regulated in the corresponding psoriasis sample (CT=33). Therefore expression of this gene can be used to distinguish between these samples. In addition, the expression of this gene is up-regulated in lung from emphysema and COPD patients, which is consistent with its expression in “stressed” small airway epithelium, lung fibroblasts and lung endothelium (treated with TNF-a and IL-1). Therapeutic modulation of the expression of this putative protein and/or signaling via this protein by antibodies, small molecules or protein therapeutics may inhibit inflammation in lung tissue clue to asthma, emphysema and other COPD type diseases

CNS_neurodegeneration_v1.0 Summary: Ag2897 This panel does not show differential expression of the CG58567-01 gene in Alzheimer's disease. However, this expression profile confirms the presence of this gene in the brain. Please see Panel 1.3D for discussion of utility of this gene in the central nervous system.

Panel 1.3D Summary: Ag2897 Two experiments with the same probe and primer show highest expression of this gene, a protocadherin homolog, in the spinal cord and the cerebral cortex (CTs=31.5). Low levels of expression are also seen in other regions of the brain including the amygdala and the hippocampus. The cadherins have been shown to be critical for CNS development, specifically for the guidance of axons, dendrites and/or growth cones in general. Therapeutic modulation of the levels of this protein, or possible signaling via this protein may be of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease). Since protocadherins play an important role in synaptogenesis this gene product may also be involved in depression, schizophrenia, which also involve synaptogeneisis. Because this cadherin shows highest expression in the cerebellum, this is also an excellent candidate for the spinocerebellar ataxias as well.

Significant levels of expression are also seen in cell lines derived from ovarian, renal and brain cancers. Thus, expression of this gene could be used to differentiate between these samples and other samples on this panel and as a marker to detect the presence of these cancers. Furthermore, therapeutic modulation of the expression or function of this gene may be effective in the treatment of ovarian, renal and brain cancers.

References:

Hilschmann N, Barnikol H U, Barnikol-Watanabe S, Gotz H, Kratzin H, Thinnes F P. The immunoglobulin-like genetic predetermination of the brain: the protocadherins, blueprint of the neuronal network. Naturwissenschaften January 2001;88(1):2-12

Panel 2D Summary: Ag2897 Two experiments with the same probe and primer produce results that are in reasonable agreement, with highest expression of the CG58567-01 gene in ovarian and kidney cancers (CTs=30.5). Significant levels of expression are also seen in lung and uterine cancers. In addition, higher levels of expression arc seen in these cancers than in the corresponding normal adjacent tissues. Thus, therapeutic targeting of this gene product with a human monoclonal antibody is anticipated to limit or block the extent of tumor cell migration and invasion, preferably in kidney, lung, uterine and ovarian tumor tumors.

Panel 3D Summary: Ag2897 Highest expression of the CG58567-01 gene is seen in a lung cancer cell line (CT=30.1). Thus, expression of this gene could be used to differentiate between this sample and other samples on this panel. Please see panel 2D for the utility of this gene.

Panel 4D Summary: Ag2897 Two experiments with the same probe and primer produce results that are in reasonable agreement, with the CG58567-01 gene highly up-regulated in small airway epithelium and astrocytes stimulated with TNF-alpha and IL-1 beta (CTs=33-34). Other tissues in the lung also up regulate the expression of this gene including lung microvascular endothelium and lung fibroblasts in response to TNF alpha or the Th2 elaborated cytokine IL-4. This suggests that this molecule could be expressed as a result of inflammation particularly during asthma since TNFalpha and IL-4 may play important roles in the pathology of this disease. Based on the expression profile of this transcript and the types of cytokines which induce it, antibodies to CG58567-01 may inhibit inflammation in lung tissue due to asthma, emphysema and other COPD type diseases.

Panel CNS_{—}1 Summary: Ag2897 The results of this panel confirm expression of the CG58567-01 gene in the brain. Please see Panel 1.3D for discussion of utility of this gene in the central nervous system.

### V. NOV28b and NOV28c (CG58567-05, CG58567-06: PROTOCADHERIN LIKE)

Expression of gene CG58567-05 and CG58567-06 was assessed using the primer-probe sets Ag2897 and Ag705, described in Tables VA and VB. Results of the RTQ-PCR runs are shown in Tables VC, VD, VE, VF, VG, VH, VI and VJ.

TABLE VA
Probe Name Ag2897

| | | | | Start |
|---|---|---|---|---|
| Primers | Sequences | | Length | Position |
| Forward | 5′-tgacaggggatattcatgctaa-3′ | (SEQ ID NO:293) | 22 | 7385 |
| Probe | TET-5′-tggcaataaatactgcctcacagtcca-3′-TAMRA | (SEQ ID NO:297) | 27 | 7428 |
| Reverse | 5′-agttgcatcacctttgtctttg-3′ | (SEQ ID NO:298) | 22 | 7458 |

TABLE VB
Probe Name Ag705

| | | | | Start |
|---|---|---|---|---|
| Primers | Sequences | | Length | Position |
| Forward | 5′-gcattcaccattgatcctatgt-3′ | (SEQ ID NO:299) | 22 | 2980 |
| Probe | TET-5′-acattgaaaaccagcaacaccctcg-3′-TAMRA | (SEQ ID NO:300) | 25 | 3007 |
| Reverse | 5′-gtatgctgagatctggcttcac-3′ | (SEQ ID NO:301) | 22 | 3035 |

TABLE VC
AI_comprehensive panel_v1.0

| | Rel. Exp.(%) Ag2897, | | Rel. Exp.(%) Ag2897, |
|---|---|---|---|
| Tissue Name | Run 229662505 | Tissue Name | Run 229662505 |
| 110967 COPD-F | 4.7 | 112427 Match Control | 100.0 |
| | | Psoriasis-F | |
| 110980 COPD-F | 22.5 | 112418 Psoriasis-M | 2.8 |
| 110968 COPD-M | 8.5 | 112723 Match Control | 14.3 |
| | | Psoriasis-M | |
| 110977 COPD-M | 51.8 | 112419 Psoriasis-M | 10.7 |
| 110989 Emphysema-F | 51.8 | 112424 Match Control | 8.7 |
| | | Psoriasis-M | |
| 110992 Emphysema-F | 5.0 | 112420 Psoriasis-M | 40.1 |
| 110993 Emphysema-F | 5.6 | 112425 Match Control | 65.1 |
| | | Psoriasis-M | |
| 110994 Emphysema-F | 3.3 | 104689 (MF) OA Bone- | 1.7 |
| | | Backus | |
| 110995 Emphysema-F | 13.2 | 104690 (MF) Adj | 4.5 |
| | | “Normal” Bone-Backus | |
| 110996 Emphysema-F | 0.6 | 104691 (MF) OA | 9.5 |
| | | Synovium-Backus | |
| 110997 Asthma-M | 1.4 | 104692 (BA) OA | 3.1 |
| | | Cartilage-Backus | |
| 111001 Asthma-F | 8.1 | 104694 (BA) OA Bone- | 1.3 |
| | | Backus | |
| 111002 Asthma-F | 12.5 | 104695 (BA) Adj | 1.8 |
| | | “Normal” Bone-Backus | |
| 111003 Atopic | 12.8 | 104696 (BA) OA | 5.0 |
| Asthma-F | | Synovium-Backus | |
| 111004 Atopic | 12.3 | 104700 (SS) OA Bone- | 5.2 |
| Asthma-F | | Backus | |
| 111005 Atopic | 6.1 | 104701 (SS) Adj | 7.0 |
| Asthma-F | | “Normal” Bone-Backus | |
| 111006 Atopic | 3.7 | 104702 (SS) OA | 12.7 |
| Asthma-F | | Synovium-Backus | |
| 111417 Allergy-M | 7.7 | 117093 OA Cartilage | 15.1 |
| | | Rep7 | |
| 112347 Allergy-M | 0.8 | 112672 OA Bone5 | 15.9 |
| 112349 Normal | 0.8 | 112673 OA Synovium5 | 8.2 |
| Lung-F | | | |
| 112357 Normal | 20.2 | 112674 OA Synovial | 7.9 |
| Lung-F | | Fluid cells5 | |
| 112354 Normal | 2.1 | 117100 OA Cartilage | 1.3 |
| Lung-M | | Rep14 | |
| 112374 Crohns-F | 4.7 | 112756 OA Bone9 | 0.8 |
| 112389 Match Control | 2.1 | 112757 OA Synovium9 | 0.4 |
| Crohns-F | | | |
| 112375 Crohns-F | 6.7 | 112758 OA Synovial | 10.2 |
| | | Fluid Cells9 | |
| 112732 Match Control | 2.2 | 117125 RA Cartilage | 6.5 |
| Crohns-F | | Rep2 | |
| 112725 Crohns-M | 18.9 | 113492 Bone2 RA | 2.2 |
| 112387 Match Control | 11.2 | 113493 Synovium2 RA | 0.2 |
| Crohns-M | | | |
| 112378 Crohns-M | 1.3 | 113494 Syn Fluid Cells | 2.0 |
| | | RA | |
| 112390 Match Control | 62.4 | 113499 Cartilage4 RA | 1.6 |
| Crohns-M | | | |
| 112726 Crohns-M | 9.6 | 113500 Bone4 RA | 1.3 |
| 112731 Match Control | 16.0 | 113501 Synovium4 RA | 1.9 |
| Crohns-M | | | |
| 112380 Ulcer Col-F | 21.2 | 113502 Syn Fluid | 1.0 |
| | | Cells4 RA | |
| 112734 Match Control | 4.5 | 113495 Cartilage3 RA | 1.1 |
| Ulcer Col-F | | | |
| 112384 Ulcer Col-F | 17.3 | 113496 Bone3 RA | 1.4 |
| 112737 Match Control | 5.2 | 113497 Synovium3 RA | 0.2 |
| Ulcer Col-F | | | |
| 112386 Ulcer Col-F | 5.3 | 113498 Syn Fluid | 1.2 |
| | | Cells3 RA | |
| 112738 Match Control | 2.7 | 117106 Normal | 0.3 |
| Ulcer Col-F | | Cartilage Rep20 | |
| 112381 Ulcer Col-M | 0.3 | 113663 Bone3 Normal | 2.8 |
| 112735 Match Control | 23.7 | 113664 Synovium3 | 0.2 |
| Ulcer Col-M | | Normal | |
| 112382 Ulcer Col-M | 5.8 | 113665 Syn Fluid | 1.3 |
| | | Cells3 Normal | |
| 112394 Match Control | 6.5 | 117107 Normal | 9.2 |
| Ulcer Col-M | | Cartilage Rep22 | |
| 112383 Ulcer Col-M | 1.6 | 113667 Bone4 Normal | 9.5 |
| 112736 Match Control | 1.3 | 113668 Synovium4 | 6.1 |
| Ulcer Col-M | | Normal | |
| 112423 Psoriasis-F | 7.9 | 113669 Syn Fluid | 10.2 |
| | | Cells4 Normal | |

TABLE VD
CNS_neurodegeneration_v1.0

| | Rel. Exp.(%) Ag2897, | | Rel. Exp.(%) Ag2897, |
|---|---|---|---|
| Tissue Name | Run 209734745 | Tissue Name | Run 209734745 |
| AD 1 Hippo | 6.5 | Control (Path) 3 | 8.9 |
| | | Temporal Ctx | |
| AD 2 Hippo | 31.9 | Control (Path) 4 | 46.7 |
| | | Temporal Ctx | |
| AD 3 Hippo | 1.6 | AD 1 Occipital Ctx | 5.6 |
| AD 4 Hippo | 7.3 | AD 2 Occipital Ctx | 0.0 |
| | | (Missing) | |
| AD 5 Hippo | 46.3 | AD 4 Occipital Ctx | 6.1 |
| AD 6 Hippo | 44.4 | AD 5 Occipital Ctx | 23.0 |
| Control 2 Hippo | 24.0 | AD 5 Occipital Ctx | 11.3 |
| Control 4 Hippo | 15.7 | AD 6 Occipital Ctx | 22.5 |
| Control (Path) 3 | 0.0 | Control 1 Occipital | 1.6 |
| Hippo | | Ctx | |
| AD 1 Temporal Ctx | 3.0 | Control 2 Occipital | 17.7 |
| | | Ctx | |
| AD 2 Temporal Ctx | 38.7 | Control 3 Occipital | 9.1 |
| | | Ctx | |
| AD 3 Temporal Ctx | 3.5 | Control 4 Occipital | 8.7 |
| | | Ctx | |
| AD 4 Temporal Ctx | 23.0 | Control (Path) 1 | 92.0 |
| | | Occipital Ctx | |
| AD 5 Inf Temporal | 79.0 | Control (Path) 2 | 0.8 |
| Ctx | | Occipital Ctx | |
| AD 5 Sup Temporal | 43.5 | Control (Path) 3 | 1.6 |
| Ctx | | Occipital Ctx | |
| AD 6 Inf Temporal | 41.8 | Control (Path) 4 | 7.6 |
| Ctx | | Occipital Ctx | |
| AD 6 Sup Temporal | 41.2 | Control 1 Parietal | 4.2 |
| Ctx | | Ctx | |
| Control 1 Temporal | 12.3 | Control 2 Parietal | 47.3 |
| Ctx | | Ctx | |
| Control 2 Temporal | 50.3 | Control 3 Parietal | 10.1 |
| Ctx | | Ctx | |
| Control 3 Temporal | 17.8 | Control (Path) 1 | 100.0 |
| Ctx | | Parietal Ctx | |
| Control 4 Temporal | 9.3 | Control (Path) 2 | 13.4 |
| Ctx | | Parietal Ctx | |
| Control (Path) 1 | 70.2 | Control (Path) 3 | 4.2 |
| Temporal Ctx | | Parietal Ctx | |
| Control (Path) 2 | 29.3 | Control (Path) 4 | 33.2 |
| Temporal Ctx | | Parietal Ctx | |

TABLE VE
Panel 1.2

| | Rel. Exp.(%) | Rel. Exp.(%) | | Rel. Exp.(%) | Rel. Exp.(%) |
|---|---|---|---|---|---|
| | Ag705, Run | Ag705, Run | | Ag705, Run | Ag705, Run |
| Tissue Name | 118753904 | 132960873 | Tissue Name | 118753904 | 132960873 |
| Endothelial cells | 0.0 | 0.0 | Renal ca. 786-0 | 0.1 | 0.0 |
| Heart (Fetal) | 0.2 | 0.0 | Renal ca. A498 | 2.7 | 0.0 |
| Pancreas | 5.0 | 0.0 | Renal ca. RXF | 0.0 | 0.0 |
| | | | 393 | | |
| Pancreatic ca. | 0.0 | 0.0 | Renal ca. | 0.0 | 0.0 |
| CAPAN 2 | | | ACHN | | |
| Adrenal Gland | 3.0 | 0.0 | Renal ca. UO- | 0.0 | 0.0 |
| | | | 31 | | |
| Thyroid | 1.2 | 0.0 | Renal ca. TK- | 0.0 | 0.0 |
| | | | 10 | | |
| Salivary gland | 0.8 | 0.0 | Liver | 0.4 | 0.0 |
| Pituitary gland | 19.8 | 0.3 | Liver(fetal) | 0.0 | 0.0 |
| Brain (fetal) | 4.9 | 0.0 | Liver ca. | 0.1 | 0.0 |
| | | | (hepatoblast) | | |
| | | | HepG2 | | |
| Brain (whole) | 27.2 | 18.4 | Lung | 0.5 | 0.0 |
| Brain (amygdala) | 5.8 | 0.8 | Lung (fetal) | 2.3 | 0.0 |
| Brain | 0.5 | 0.0 | Lung ca. (small | 0.0 | 0.0 |
| (cerebellum) | | | cell) LX-1 | | |
| Brain | 21.9 | 7.9 | Lung ca. (small | 0.0 | 0.0 |
| (hippocampus) | | | cell) NCI-H69 | | |
| Brain (thalamus) | 2.8 | 0.0 | Lung ca. (s.cell | 0.0 | 0.0 |
| | | | var.) SHP-77 | | |
| Cerebral Cortex | 56.6 | 100.0 | Lung ca. (large | 0.0 | 0.0 |
| | | | cell) NCI-H460 | | |
| Spinal cord | 32.8 | 11.4 | Lung ca. (non- | 0.0 | 0.0 |
| | | | sm. cell) A549 | | |
| glio/astro U87 | 62.4 | 19.1 | Lung ca. (non- | 0.2 | 0.0 |
| MG | | | s.cell) NCI- | | |
| | | | H23 | | |
| glio/astro U-118- | 0.2 | 0.0 | Lung ca. (non- | 1.6 | 0.0 |
| MG | | | s.cell) HOP-62 | | |
| astrocytoma | 0.1 | 0.0 | Lung ca. (non- | 0.0 | 0.0 |
| SW1783 | | | s.cl) NCI-H522 | | |
| neuro*; met SK- | 0.0 | 0.0 | Lung ca. | 0.5 | 0.0 |
| N-AS | | | (squam.) SW | | |
| | | | 900 | | |
| astrocytoma SF- | 0.1 | 0.0 | Lung ca. | 0.0 | 0.0 |
| 539 | | | (squam.) NCI- | | |
| | | | H596 | | |
| astrocytoma | 34.9 | 7.6 | Mammary | 14.2 | 5.4 |
| SNB-75 | | | gland | | |
| glioma SNB-19 | 0.4 | 0.0 | Breast ca.* | 0.0 | 0.0 |
| | | | (pl.ef) MCF-7 | | |
| glioma U251 | 0.1 | 0.0 | Breast ca.* | 0.0 | 0.0 |
| | | | (pl.ef) MDA- | | |
| | | | MB-231 | | |
| glioma SF-295 | 0.4 | 0.0 | Breast ca.* (pl. | 0.7 | 0.0 |
| | | | ef) T47D | | |
| Heart | 0.1 | 0.0 | Breast ca. BT- | 0.4 | 0.0 |
| | | | 549 | | |
| Skeletal Muscle | 1.0 | 0.0 | Breast ca. | 0.0 | 0.0 |
| | | | MDA-N | | |
| Bone marrow | 0.0 | 0.0 | Ovary | 2.0 | 0.1 |
| Thymus | 0.0 | 0.0 | Ovarian ca. | 14.5 | 0.9 |
| | | | OVCAR-3 | | |
| Spleen | 0.0 | 0.0 | Ovarian ca. | 0.0 | 0.0 |
| | | | OVCAR-4 | | |
| Lymph node | 0.6 | 0.0 | Ovarian ca. | 100.0 | 9.3 |
| | | | OVCAR-5 | | |
| Colorectal Tissue | 1.2 | 0.1 | Ovarian ca. | 0.4 | 0.0 |
| | | | OVCAR-8 | | |
| Stomach | 35.4 | 15.9 | Ovarian ca. | 0.0 | 0.0 |
| | | | IGROV-1 | | |
| Small intestine | 5.7 | 0.0 | Ovarian ca. | 0.0 | 0.0 |
| | | | (ascites) SK- | | |
| | | | OV-3 | | |
| Colon ca. | 0.0 | 0.0 | Uterus | 5.0 | 2.0 |
| SW480 | | | | | |
| Colon ca.* | 0.0 | 0.0 | Placenta | 0.2 | 0.0 |
| SW620 (SW480 | | | | | |
| met) | | | | | |
| Colon ca. HT29 | 0.0 | 0.0 | Prostate | 2.6 | 0.5 |
| Colon ca. HCT- | 0.0 | 0.0 | Prostate ca.* | 0.0 | 0.0 |
| 116 | | | (bone met) | | |
| | | | PC-3 | | |
| Colon ca. CaCo-2 | 0.1 | 0.0 | Testis | 96.6 | 9.5 |
| Colon ca. Tissue | 0.0 | 0.0 | Melanoma | 0.1 | 0.0 |
| (ODO3866) | | | Hs688(A).T | | |
| Colon ca. HCC- | 0.1 | 0.0 | Melanoma* | 4.2 | 0.0 |
| 2998 | | | (met) | | |
| | | | Hs688(B).T | | |
| Gastric ca.* | 7.2 | 0.0 | Melanoma | 0.1 | 0.0 |
| (liver met) NCI- | | | UACC-62 | | |
| N87 | | | | | |
| Bladder | 5.2 | 0.1 | Melanoma | 0.0 | 0.0 |
| | | | M14 | | |
| Trachea | 1.0 | 0.5 | Melanoma | 0.0 | 0.0 |
| | | | LOX IMVI | | |
| Kidney | 1.5 | 0.0 | Melanoma* | 0.0 | 0.0 |
| | | | (met) SK- | | |
| | | | MEL-5 | | |
| Kidney (fetal) | 6.4 | 0.2 | | | |

TABLE VF
Panel 1.3D

| | Rel. Exp.(%) | Rel. Exp.(%) | | Rel. Exp.(%) | Rel. Exp.(%) |
|---|---|---|---|---|---|
| | Ag2897, Run | Ag2897, Run | | Ag2897, Run | Ag2897, Run |
| Tissue Name | 161905860 | 165518166 | Tissue Name | 161905860 | 165518166 |
| Liver | 0.0 | 0.0 | Kidney (fetal) | 2.8 | 2.4 |
| adenocarcinoma | | | | | |
| Pancreas | 1.4 | 2.4 | Renal ca. 786-0 | 0.9 | 3.3 |
| Pancreatic ca. | 0.0 | 0.0 | Renal ca. A498 | 13.9 | 23.7 |
| CAPAN 2 | | | | | |
| Adrenal gland | 1.4 | 0.0 | Renal ca. RXF | 0.0 | 0.0 |
| | | | 393 | | |
| Thyroid | 0.0 | 0.0 | Renal ca. | 0.0 | 0.0 |
| | | | ACHN | | |
| Salivary gland | 0.0 | 0.0 | Renal ca. UO- | 0.0 | 0.0 |
| | | | 31 | | |
| Pituitary gland | 4.1 | 9.0 | Renal ca. TK- | 0.0 | 0.0 |
| | | | 10 | | |
| Brain (fetal) | 7.1 | 17.8 | Liver | 0.0 | 2.9 |
| Brain (whole) | 21.0 | 59.0 | Liver (fetal) | 0.4 | 0.0 |
| Brain (amygdala) | 24.7 | 37.9 | Liver ca. | 0.0 | 0.0 |
| | | | (hepatoblast) | | |
| | | | HepG2 | | |
| Brain (cerebellum) | 5.6 | 28.5 | Lung | 1.4 | 0.0 |
| Brain | 25.2 | 25.3 | Lung (fetal) | 6.8 | 11.0 |
| (hippocampus) | | | | | |
| Brain (substantia | 0.9 | 17.1 | Lung ca. | 0.0 | 0.0 |
| nigra) | | | (small cell) | | |
| | | | LX-1 | | |
| Brain (thalamus) | 4.1 | 23.0 | Lung ca. | 0.0 | 4.1 |
| | | | (small cell) | | |
| | | | NCI-H69 | | |
| Cerebral Cortex | 100.0 | 22.2 | Lung ca. (s.cell | 1.0 | 0.0 |
| | | | var:) SHP-77 | | |
| Spinal cord | 68.8 | 100.0 | Lung ca. (large | 0.0 | 0.0 |
| | | | cell) NCI-H460 | | |
| glio/astro U87-MG | 54.3 | 10.4 | Lung ca. (non- | 0.0 | 0.0 |
| | | | sm. cell) A549 | | |
| glio/astro U-118- | 1.5 | 20.4 | Lung ca. (non- | 0.0 | 3.0 |
| MG | | | s.cell) NCI- | | |
| | | | H23 | | |
| astrocytoma | 16.3 | 0.0 | Lung ca. (non- | 0.6 | 7.5 |
| SW1783 | | | s.cell) HOP-62 | | |
| neuro*; met SK-N- | 0.0 | 0.0 | Lung ca. (non- | 0.0 | 0.0 |
| AS | | | s.cl) NCI-H522 | | |
| astrocytoma SF- | 0.0 | 4.3 | Lung ca. | 4.6 | 8.6 |
| 539 | | | (squam.) SW | | |
| | | | 900 | | |
| astrocytoma SNB- | 18.4 | 28.7 | Lung ca. | 0.0 | 0.0 |
| 75 | | | (squam.) NCI- | | |
| | | | H596 | | |
| glioma SNB-19 | 0.0 | 2.6 | Mammary | 7.9 | 17.1 |
| | | | gland | | |
| glioma U251 | 3.1 | 27.0 | Breast ca.* | 0.0 | 0.0 |
| | | | (pl.ef) MCF-7 | | |
| glioma SF-295 | 4.8 | 2.2 | Breast ca.* | 0.0 | 14.5 |
| | | | (pl.ef) MDA- | | |
| | | | MB-231 | | |
| Heart (fetal) | 0.0 | 0.0 | Breast ca.* | 0.0 | 0.0 |
| | | | (pl.ef) T47D | | |
| Heart | 1.4 | 0.0 | Breast ca. BT- | 0.0 | 12.2 |
| | | | 549 | | |
| Skeletal muscle | 77.4 | 0.0 | Breast ca. | 0.0 | 0.0 |
| (fetal) | | | MDA-N | | |
| Skeletal muscle | 1.1 | 14.1 | Ovary | 12.1 | 6.1 |
| Bone marrow | 0.0 | 2.5 | Ovarian ca. | 3.8 | 12.1 |
| | | | OVCAR-3 | | |
| Thymus | 2.1 | 2.4 | Ovarian ca. | 0.0 | 0.0 |
| | | | OVCAR-4 | | |
| Spleen | 0.0 | 4.4 | Ovarian ca. | 32.5 | 25.5 |
| | | | OVCAR-5 | | |
| Lymph node | 0.0 | 4.8 | Ovarian ca. | 1.9 | 0.0 |
| | | | OVCAR-8 | | |
| Colorectal | 10.7 | 0.0 | Ovarian ca. | 0.0 | 0.0 |
| | | | IGROV-1 | | |
| Stomach | 8.7 | 64.6 | Ovarian ca.* | 0.0 | 0.0 |
| | | | (ascites) SK- | | |
| | | | OV-3 | | |
| Small intestine | 7.2 | 18.9 | Uterus | 12.4 | 66.4 |
| Colon ca. SW480 | 0.0 | 0.0 | Placenta | 0.0 | 2.3 |
| Colon ca.* | 0.0 | 0.0 | Prostate | 0.0 | 0.0 |
| SW620(SW480 | | | | | |
| met) | | | | | |
| Colon ca. HT29 | 0.0 | 0.0 | Prostate ca.* | 0.0 | 0.0 |
| | | | (bone met)PC-3 | | |
| Colon ca. HCT- | 0.0 | 0.0 | Testis | 56.3 | 68.8 |
| 116 | | | | | |
| Colon ca. CaCo-2 | 0.0 | 0.0 | Melanoma | 1.3 | 8.2 |
| | | | Hs688(A).T | | |
| Colon ca. | 1.7 | 8.1 | Melanoma* | 2.5 | 4.8 |
| tissue(ODO3866) | | | (met) | | |
| | | | Hs688(B).T | | |
| Colon ca. HCC- | 0.0 | 0.0 | Melanoma | 1.5 | 2.8 |
| 2998 | | | UACC-62 | | |
| Gastric ca.* (liver | 10.3 | 7.7 | Melanoma | 0.0 | 0.0 |
| met) NCI-N87 | | | M14 | | |
| Bladder | 14.2 | 29.3 | Melanoma | 0.0 | 0.0 |
| | | | LOX IMVI | | |
| Trachea | 4.1 | 0.0 | Melanoma* | 0.0 | 0.0 |
| | | | (met) SK- | | |
| | | | MEL-5 | | |
| Kidney | 0.0 | 0.0 | Adipose | 7.2 | 0.0 |

TABLE VG
Panel 2D

| | Rel. Exp.(%) | Rel. Exp.(%) | | Rel. Exp.(%) | Rel. Exp.(%) |
|---|---|---|---|---|---|
| | Ag2897, Run | Ag2897, Run | | Ag2897, Run | Ag2897, Run |
| Tissue Name | 161905861 | 164997312 | Tissue Name | 161905861 | 164997312 |
| Normal Colon | 83.5 | 90.1 | Kidney Margin | 1.3 | 0.9 |
| | | | 8120608 | | |
| CC Well to Mod | 0.0 | 7.2 | Kidney Cancer | 1.4 | 1.6 |
| Diff (ODO3866) | | | 8120613 | | |
| CC Margin | 16.0 | 16.6 | Kidney Margin | 0.9 | 1.5 |
| (ODO3866) | | | 8120614 | | |
| CC Gr.2 | 0.0 | 0.0 | Kidney Cancer | 21.6 | 16.0 |
| rectosigmoid | | | 9010320 | | |
| (ODO3868) | | | | | |
| CC Margin | 6.3 | 3.6 | Kidney Margin | 1.9 | 0.5 |
| (ODO3868) | | | 9010321 | | |
| CC Mod Diff | 0.9 | 2.1 | Normal Uterus | 9.5 | 10.9 |
| (ODO3920) | | | | | |
| CC Margin | 19.1 | 11.7 | Uterus Cancer | 68.3 | 71.2 |
| (ODO3920) | | | 064011 | | |
| CC Gr.2 ascend | 8.8 | 9.2 | Normal | 3.3 | 0.8 |
| colon | | | Thyroid | | |
| (ODO3921) | | | | | |
| CC Margin | 6.9 | 7.5 | Thyroid | 2.0 | 6.9 |
| (ODO3921) | | | Cancer 064010 | | |
| CC from Partial | 0.9 | 3.3 | Thyroid | 8.9 | 12.2 |
| Hepatectomy | | | Cancer | | |
| (ODO4309) Mets | | | A302152 | | |
| Liver Margin | 0.7 | 2.0 | Thyroid | 1.0 | 0.7 |
| (ODO4309) | | | Margin | | |
| | | | A302153 | | |
| Colon mets to | 0.0 | 0.0 | Normal Breast | 15.9 | 15.3 |
| lung (OD04451- | | | | | |
| 01) | | | | | |
| Lung Margin | 1.9 | 1.0 | Breast Cancer | 4.3 | 6.3 |
| (OD04451-02) | | | (OD04566) | | |
| Normal Prostate | 3.9 | 10.7 | Breast Cancer | 7.3 | 2.8 |
| 6546-1 | | | (OD04590-01) | | |
| Prostate Cancer | 17.3 | 11.6 | Breast Cancer | 9.4 | 4.8 |
| (OD04410) | | | Mets | | |
| | | | (OD04590-03) | | |
| Prostate Margin | 25.0 | 15.5 | Breast Cancer | 3.6 | 0.9 |
| (OD04410) | | | Metastasis | | |
| | | | (OD04655-05) | | |
| Prostate Cancer | 28.3 | 11.7 | Breast Cancer | 8.4 | 11.6 |
| (OD04720-01) | | | 064006 | | |
| Prostate Margin | 29.9 | 20.4 | Breast Cancer | 2.6 | 4.3 |
| (OD04720-02) | | | 1024 | | |
| Normal Lung | 16.2 | 15.1 | Breast Cancer | 1.9 | 2.0 |
| 061010 | | | 9100266 | | |
| Lung Met to | 1.2 | 0.0 | Breast Margin | 7.4 | 5.4 |
| Muscle | | | 9100265 | | |
| (ODO4286) | | | | | |
| Muscle Margin | 8.2 | 1.6 | Breast Cancer | 20.0 | 13.6 |
| (ODO4286) | | | A209073 | | |
| Lung Malignant | 7.0 | 2.2 | Breast Margin | 5.0 | 12.0 |
| Cancer | | | A209073 | | |
| (OD03126) | | | | | |
| Lung Margin | 2.3 | 3.0 | Normal Liver | 0.9 | 1.0 |
| (OD03126) | | | | | |
| Lung Cancer | 8.2 | 3.1 | Liver Cancer | 0.0 | 0.0 |
| (OD04404) | | | 064003 | | |
| Lung Margin | 11.1 | 10.2 | Liver Cancer | 2.4 | 0.0 |
| (OD04404) | | | 1025 | | |
| Lung Cancer | 0.0 | 0.9 | Liver Cancer | 0.0 | 0.0 |
| (OD04565) | | | 1026 | | |
| Lung Margin | 1.9 | 5.0 | Liver Cancer | 0.0 | 1.0 |
| (OD04565) | | | 6004-T | | |
| Lung Cancer | 95.3 | 55.9 | Liver Tissue | 0.0 | 0.0 |
| (OD04237-01) | | | 6004-N | | |
| Lung Margin | 4.0 | 2.3 | Liver Cancer | 0.0 | 0.0 |
| (OD04237-02) | | | 6005-T | | |
| Ocular Mel Met | 0.0 | 0.0 | Liver Tissue | 0.0 | 0.0 |
| to Liver | | | 6005-N | | |
| (ODO4310) | | | | | |
| Liver Margin | 2.7 | 0.8 | Normal | 18.0 | 13.8 |
| (ODO4310) | | | Bladder | | |
| Melanoma Mets | 0.0 | 1.7 | Bladder | 1.0 | 0.7 |
| to Lung | | | Cancer 1023 | | |
| (OD04321) | | | | | |
| Lung Margin | 10.1 | 6.6 | Bladder | 10.9 | 2.6 |
| (OD04321) | | | Cancer | | |
| | | | A302173 | | |
| Normal Kidney | 14.8 | 16.4 | Bladder | 0.0 | 1.9 |
| | | | Cancer | | |
| | | | (OD04718-01) | | |
| Kidney Ca, | 93.3 | 100.0 | Bladder | 33.2 | 31.0 |
| Nuclear grade 2 | | | Normal | | |
| (OD04338) | | | Adjacent | | |
| | | | (OD04718-03) | | |
| Kidney Margin | 14.3 | 2.0 | Normal Ovary | 0.0 | 4.8 |
| (OD04338) | | | | | |
| Kidney Ca | 5.0 | 2.0 | Ovarian | 100.0 | 62.0 |
| Nuclear grade 1/2 | | | Cancer 064008 | | |
| (OD04339) | | | | | |
| Kidney Margin | 4.1 | 3.0 | Ovarian | 49.7 | 38.7 |
| (OD04339) | | | Cancer | | |
| | | | (OD04768-07) | | |
| Kidney Ca, Clear | 27.0 | 17.4 | Ovary Margin | 6.3 | 2.5 |
| cell type | | | (OD04768-08) | | |
| (OD04340) | | | | | |
| Kidney Margin | 6.4 | 12.7 | Normal | 28.1 | 18.7 |
| (OD04340) | | | Stomach | | |
| Kidney Ca, | 1.9 | 2.1 | Gastric Cancer | 11.0 | 14.1 |
| Nuclear grade 3 | | | 9060358 | | |
| (OD04348) | | | | | |
| Kidney Margin | 4.7 | 6.2 | Stomach | 15.9 | 29.7 |
| (OD04348) | | | Margin | | |
| | | | 9060359 | | |
| Kidney Cancer | 50.0 | 38.4 | Gastric Cancer | 15.0 | 6.8 |
| (OD04622-01) | | | 9060395 | | |
| Kidney Margin | 4.2 | 0.0 | Stomach | 26.6 | 26.4 |
| (OD04622-03) | | | Margin | | |
| | | | 9060394 | | |
| Kidney Cancer | 2.6 | 3.6 | Gastric Cancer | 2.7 | 1.7 |
| (OD04450-01) | | | 9060397 | | |
| Kidney Margin | 14.2 | 10.5 | Stomach | 28.1 | 33.0 |
| (OD04450-03) | | | Margin | | |
| | | | 9060396 | | |
| Kidney Cancer | 1.9 | 0.0 | Gastric Cancer | 27.7 | 33.4 |
| 8120607 | | | 064005 | | |

TABLE VH
Panel 3D

| | Rel. Exp.(%) | | Rel. Exp.(%) |
|---|---|---|---|
| | Ag2897, Run | | Ag2897, Run |
| Tissue Name | 164629841 | Tissue Name | 164629841 |
| Daoy-Medulloblastoma | 0.0 | Ca Ski-Cervical epidermoid | 0.0 |
| | | carcinoma (metastasis) | |
| TE671-Medulloblastoma | 0.0 | ES-2-Ovarian clear cell | 0.8 |
| | | carcinoma | |
| D283 Med- | 0.0 | Ramos-Stimulated with | 0.0 |
| Medulloblastoma | | PMA/ionomycin 6h | |
| PFSK-1-Primitive | 0.0 | Ramos-Stimulated with | 0.0 |
| Neuroectodermal | | PMA/ionomycin 14h | |
| XF-498-CNS | 0.6 | MEG-01-Chronic | 0.0 |
| | | myelogenous leukemia | |
| | | (megokaryoblast) | |
| SNB-78-Glioma | 6.0 | Raji-Burkitt's lymphoma | 0.0 |
| SF-268-Glioblastoma | 0.0 | Daudi-Burkitt's lymphoma | 0.0 |
| T98G-Glioblastoma | 0.0 | U266-B-cell plasmacytoma | 0.0 |
| SK-N-SH- | 0.0 | CA46-Burkitt's lymphoma | 0.0 |
| Neuroblastoma | | | |
| (metastasis) | | | |
| SF-295-Glioblastoma | 0.0 | RL-non-Hodgkin's B-cell | 0.0 |
| | | lymphoma | |
| Cerebellum | 7.1 | JM1-pre-B-cell lymphoma | 0.0 |
| Cerebellum | 0.0 | Jurkat-T cell leukemia | 0.0 |
| NCI-H292- | 0.0 | TF-1-Erythroleukemia | 0.0 |
| Mucoepidermoid lung | | | |
| carcinoma | | | |
| DMS-114-Small cell | 0.0 | HUT 78-T-cell lymphoma | 0.0 |
| lung cancer | | | |
| DMS-79-Small cell lung | 0.8 | U937-Histiocytic lymphoma | 0.0 |
| cancer | | | |
| NCI-H146-Small cell | 1.3 | KU-812-Myelogenous | 0.0 |
| lung cancer | | leukemia | |
| NCI-H526-Small cell | 100.0 | 769-P-Clear cell renal | 0.0 |
| lung cancer | | carcinoma | |
| NCI-N417-Small cell | 0.0 | Caki-2-Clear cell renal | 0.9 |
| lung cancer | | carcinoma | |
| NCI-H82-Small cell lung | 0.0 | SW 839-Clear cell renal | 0.0 |
| cancer | | carcinoma | |
| NCI-H157-Squamous | 0.0 | G401-Wilms' tumor | 0.0 |
| cell lung cancer | | | |
| (metastasis) | | | |
| NCI-H1155-Large cell | 3.2 | Hs766T-Pancreatic carcinoma | 0.0 |
| lung cancer | | (LN metastasis) | |
| NCI-H1299-Large cell | 1.9 | CAPAN-1-Pancreatic | 0.0 |
| lung cancer | | adenocarcinoma (liver | |
| | | metastasis) | |
| NCI-H727-Lung | 0.0 | SU86.86-Pancreatic | 0.6 |
| carcinoid | | carcinoma (liver metastasis) | |
| NCI-UMC-11-Lung | 2.1 | BxPC-3-Pancreatic | 0.0 |
| carcinoid | | adenocarcinoma | |
| LX-1-Small cell lung | 0.0 | HPAC-Pancreatic | 0.0 |
| cancer | | adenocarcinoma | |
| Colo-205-Colon cancer | 0.0 | MIA PaCa-2-Pancreatic | 0.0 |
| | | carcinoma | |
| KM12-Colon cancer | 2.6 | CFPAC-1-Pancreatic ductal | 0.4 |
| | | adenocarcinoma | |
| KM20L2-Colon cancer | 0.0 | PANC-1-Pancreatic | 0.0 |
| | | epithelioid ductal carcinoma | |
| NCI-H716-Colon cancer | 31.0 | T24-Bladder carcinma | 0.0 |
| | | (transitional cell) | |
| SW-48-Colon | 0.0 | 5637-Bladder carcinoma | 0.0 |
| adenocarcinoma | | | |
| SW1116-Colon | 0.0 | HT-1197-Bladder carcinoma | 0.0 |
| adenocarcinoma | | | |
| LS 174T-Colon | 0.0 | UM-UC-3-Bladder carcinma | 0.0 |
| adenocarcinoma | | (transitional cell) | |
| SW-948-Colon | 0.0 | A204-Rhabdomyosarcoma | 0.0 |
| adenocarcinoma | | | |
| SW-480-Colon | 1.5 | HT-1080-Fibrosarcoma | 0.8 |
| adenocarcinoma | | | |
| NCI-SNU-5-Gastric | 0.0 | MG-63-Osteosarcoma | 0.0 |
| carcinoma | | | |
| KATO III-Gastric | 0.0 | SK-LMS-1-Leiomyosarcoma | 0.0 |
| carcinoma | | (vulva) | |
| NCI-SNU-16-Gastric | 0.8 | SJRH30-Rhabdomyosarcoma | 0.0 |
| carcinoma | | (met to bone marrow) | |
| NCI-SNU-1-Gastric | 0.0 | A431-Epidermoid carcinoma | 0.0 |
| carcinoma | | | |
| RF-1-Gastric | 0.0 | WM266-4-Melanoma | 1.2 |
| adenocarcinorna | | | |
| RF-48-Gastric | 0.8 | DU 145-Prostate carcinoma | 0.0 |
| adenocarcinoma | | (brain metastasis) | |
| MKN-45-Gastric | 0.0 | MDA-MB-468-Breast | 0.8 |
| carcinoma | | adenocarcinoma | |
| NCI-N87-Gastric | 0.3 | SCC-4-Squamous cell | 0.0 |
| carcinoma | | carcinoma of tongue | |
| OVCAR-5-Ovarian | 0.0 | SCC-9-Squamous cell | 0.0 |
| carcinoma | | carcinoma of tongue | |
| RL95-2-Uterine | 0.0 | SCC-15-Squamous cell | 0.0 |
| carcinoma | | carcinoma of tongue | |
| HelaS3-Cervical | 0.0 | CAL 27-Squamous cell | 0.0 |
| adenocarcinoma | | carcinoma of tongue | |

TABLE VI
Panel 4D

| | Rel. | Rel. | | Rel. | Rel. |
|---|---|---|---|---|---|
| | Exp.(%) | Exp.(%) | | Exp.(%) | Exp.(%) |
| | Ag2897, | Ag2897, | | Ag2897, | Ag2897, |
| | Run | Run | | Run | Run |
| Tissue Name | 161905862 | 163586314 | Tissue Name | 161905862 | 163586314 |
| Secondary Th1 act | 0.0 | 0.0 | HUVEC IL-1 beta | 0.0 | 0.0 |
| Secondary Th2 act | 0.0 | 0.0 | HUVEC IFN | 0.0 | 0.0 |
| | | | gamma | | |
| Secondary Tr1 act | 0.0 | 0.0 | HUVEC TNF | 0.0 | 0.0 |
| | | | alpha + IFN | | |
| | | | gamma | | |
| Secondary Th1 rest | 0.0 | 0.0 | HUVEC TNF | 0.0 | 0.0 |
| | | | alpha + IL4 | | |
| Secondary Th2 rest | 0.0 | 0.0 | HUVEC IL-11 | 0.0 | 0.0 |
| Secondary Tr1 rest | 0.0 | 0.0 | Lung | 0.0 | 0.0 |
| | | | Microvascular EC | | |
| | | | none | | |
| Primary Th1 act | 0.0 | 0.0 | Lung | 7.9 | 22.8 |
| | | | Microvascular EC | | |
| | | | TNF alpha + IL- | | |
| | | | 1 beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 16.2 | 0.0 |
| | | | Dermal EC | | |
| | | | TNF alpha + IL- | | |
| | | | 1 beta | | |
| Primary Th1 rest | 9.3 | 20.7 | Bronchial | 4.4 | 0.0 |
| | | | epithelium | | |
| | | | TNF alpha + | | |
| | | | IL1 beta | | |
| Primary Th2 rest | 0.0 | 9.0 | Small airway | 10.4 | 9.7 |
| | | | epithelium none | | |
| Primary Tr1 rest | 9.8 | 0.0 | Small airway | 100.0 | 75.8 |
| | | | epithelium | | |
| | | | TNF alpha + IL- | | |
| | | | 1 beta | | |
| CD45RA CD4 | 0.0 | 0.0 | Coronery artery | 0.0 | 9.9 |
| lymphocyte act | | | SMC rest | | |
| CD45RO CD4 | 0.0 | 0.0 | Coronery artery | 16.8 | 9.0 |
| lymphocyte act | | | SMC TNF alpha + | | |
| | | | IL-1 beta | | |
| CD8 lymphocyte | 0.0 | 0.0 | Astrocytes rest | 40.6 | 0.0 |
| act | | | | | |
| Secondary CD8 | 0.0 | 0.0 | Astrocytes | 56.6 | 100.0 |
| lymphocyte rest | | | TNF alpha + IL- | | |
| | | | 1 beta | | |
| Secondary CD8 | 0.0 | 0.0 | KU-812 (Basophil) | 0.0 | 0.0 |
| lymphocyte act | | | rest | | |
| CD4 lymphocyte | 0.0 | 0.0 | KU-812 (Basophil) | 0.0 | 0.0 |
| none | | | PMA/ionomycin | | |
| 2ry | 0.0 | 0.0 | CCD1106 | 0.0 | 0.0 |
| Th1/Th2/Tr1_anti- | | | (Keratinocytes) | | |
| CD95 Ch11 | | | none | | |
| LAK cells rest | 10.9 | 0.0 | CCD1106 | 0.0 | 0.0 |
| | | | (Keratinocytes) | | |
| | | | TNF alpha + IL- | | |
| | | | 1 beta | | |
| LAK cells IL-2 | 0.0 | 0.0 | Liver cirrhosis | 17.2 | 8.2 |
| LAK cells IL-2 + | 0.0 | 0.0 | Lupus kidney | 8.4 | 9.6 |
| IL-12 | | | | | |
| LAK cells IL- | 0.0 | 0.0 | NCI-H292 none | 0.0 | 0.0 |
| 2 + IFN gamma | | | | | |
| LAK cells IL-2 + | 0.0 | 0.0 | NCI-H292 IL-4 | 0.0 | 0.0 |
| IL-18 | | | | | |
| LAK cells | 0.0 | 0.0 | NCI-H292 IL-9 | 0.0 | 0.0 |
| PMA/ionomycin | | | | | |
| NK Cells IL-2 rest | 0.0 | 0.0 | NCI-H292 IL-13 | 0.0 | 0.0 |
| Two Way MLR 3 | 0.0 | 9.3 | NCI-H292 IFN | 0.0 | 0.0 |
| day | | | gamma | | |
| Two Way MLR 5 | 0.0 | 0.0 | HPAEC none | 0.0 | 0.0 |
| day | | | | | |
| Two Way MLR 7 | 0.0 | 0.0 | HPAEC TNF | 0.0 | 0.0 |
| day | | | alpha + IL-1 beta | | |
| PBMC rest | 0.0 | 0.0 | Lung fibroblast | 0.0 | 0.0 |
| | | | none | | |
| PBMC PWM | 0.0 | 0.0 | Lung fibroblast | 0.0 | 18.8 |
| | | | TNF alpha + IL-1 | | |
| | | | beta | | |
| PBMC PHA-L | 8.9 | 0.0 | Lung fibroblast IL-4 | 0.0 | 17.7 |
| Ramos (B cell) | 0.0 | 0.0 | Lung fibroblast IL-9 | 0.0 | 0.0 |
| none | | | | | |
| Ramos (B cell) | 0.0 | 0.0 | Lung fibroblast IL- | 0.0 | 0.0 |
| ionomycin | | | 13 | | |
| B lymphocytes | 0.0 | 0.0 | Lung fibroblast | 0.0 | 0.0 |
| PWM | | | IFN gamma | | |
| B lymphocytes | 0.0 | 0.0 | Dermal fibroblast | 18.7 | 5.1 |
| CD40L and IL-4 | | | CCD1070 rest | | |
| EOL-1 dbcAMP | 0.0 | 0.0 | Dermal fibroblast | 45.4 | 29.5 |
| | | | CCD1070 TNF | | |
| | | | alpha | | |
| EOL-1 dbcAMP | 0.0 | 0.0 | Dermal fibroblast | 33.7 | 85.9 |
| PMA/ionomycin | | | CCD1070 IL-1 | | |
| | | | beta | | |
| Dendritic cells none | 0.0 | 0.0 | Dermal fibroblast | 0.0 | 0.0 |
| | | | IFN gamma | | |
| Dendritic cells LPS | 0.0 | 0.0 | Dermal fibroblast | 0.0 | 8.4 |
| | | | IL-4 | | |
| Dendritic cells anti- | 0.0 | 0.0 | IBD Colitis 2 | 0.0 | 9.9 |
| CD40 | | | | | |
| Monocytes rest | 0.0 | 0.0 | IBD Crohn's | 7.7 | 0.0 |
| Monocytes LPS | 0.0 | 0.0 | Colon | 62.4 | 39.2 |
| Macrophages rest | 0.0 | 0.0 | Lung | 9.9 | 22.1 |
| Macrophages LPS | 0.0 | 0.0 | Thymus | 27.7 | 35.8 |
| HUVEC none | 0.0 | 0.0 | Kidney | 18.9 | 20.6 |
| HUVEC starved | 0.0 | 0.0 | | | |

TABLE VJ
Panel CNS_1

| | Rel. Exp.(%) | | Rel. Exp.(%) |
|---|---|---|---|
| | Ag2897, Run | | Ag2897, Run |
| Tissue Name | 171688457 | Tissue Name | 171688457 |
| BA4 Control | 29.7 | BA17 PSP | 24.3 |
| BA4 Control2 | 37.6 | BA17 PSP2 | 0.0 |
| BA4 | 0.0 | Sub Nigra Control | 25.9 |
| Alzheimer's2 | | | |
| BA4 Parkinson's | 23.8 | Sub Nigra Control2 | 33.4 |
| BA4 | 38.7 | Sub Nigra | 0.0 |
| Parkinson's2 | | Alzheimer's2 | |
| BA4 | 18.4 | Sub Nigra | 26.6 |
| Huntington's | | Parkinson's2 | |
| BA4 | 0.0 | Sub Nigra | 13.9 |
| Huntington's2 | | Huntington's | |
| BA4 PSP | 8.0 | Sub Nigra | 8.7 |
| | | Huntington's2 | |
| BA4 PSP2 | 37.6 | Sub Nigra PSP2 | 0.0 |
| BA4 Depression | 15.6 | Sub Nigra | 0.0 |
| | | Depression | |
| BA4 | 4.6 | Sub Nigra | 2.8 |
| Depression2 | | Depression2 | |
| BA7 Control | 56.6 | Glob Palladus | 4.1 |
| | | Control | |
| BA7 Control2 | 6.0 | Glob Palladus | 23.7 |
| | | Control2 | |
| BA7 | 7.3 | Glob Palladus | 3.8 |
| Alzheimer's2 | | Alzheimer's | |
| BA7 Parkinson's | 10.8 | Glob Palladus | 0.0 |
| | | Alzheimer's2 | |
| BA7 | 27.2 | Glob Palladus | 51.1 |
| Parkinson's2 | | Parkinson's | |
| BA7 | 56.3 | Glob Palladus | 3.5 |
| Huntington's | | Parkinson's2 | |
| BA7 | 10.8 | Glob Palladus PSP | 0.0 |
| Huntington's2 | | | |
| BA7 PSP | 21.5 | Glob Palladus PSP2 | 0.0 |
| BA7 PSP2 | 21.3 | Glob Palladus | 0.0 |
| | | Depression | |
| BA7 Depression | 5.4 | Temp Pole Control | 15.9 |
| BA9 Control | 31.6 | Temp Pole Control2 | 37.9 |
| BA9 Control2 | 100.0 | Temp Pole | 0.0 |
| | | Alzheimer's | |
| BA9 Alzheimer's | 3.8 | Temp Pole | 0.0 |
| | | Alzheimer's2 | |
| BA9 | 3.7 | Temp Pole | 34.2 |
| Alzheimer's2 | | Parkinson's | |
| BA9 Parkinson's | 19.2 | Temp Pole | 10.8 |
| | | Parkinson's2 | |
| BA9 | 30.6 | Temp Pole | 17.7 |
| Parkinson's2 | | Huntington's | |
| BA9 | 27.2 | Temp Pole PSP | 5.8 |
| Huntington's | | | |
| BA9 | 9.0 | Temp Pole PSP2 | 0.0 |
| Huntington's | | | |
| BA9 PSP | 0.0 | Temp Pole | 15.3 |
| | | Depression2 | |
| BA9 PSP2 | 16.5 | Cing Gyr Control | 76.3 |
| BA9 Depression | 2.7 | Cing Gyr Control2 | 54.0 |
| BA9 | 6.7 | Cing Gyr | 12.1 |
| Depression2 | | Alzheimer's | |
| BA17 Control | 25.9 | Cing Gyr | 2.9 |
| | | Alzheimer's2 | |
| BA17 Control2 | 14.8 | Cing Gyr | 26.6 |
| | | Parkinson's | |
| BA17 | 7.9 | Cing Gyr | 38.2 |
| Alzheimer's2 | | Parkinson's2 | |
| BA17 | 9.5 | Cing Gyr | 36.1 |
| Parkinson's | | Huntington's | |
| BA17 | 8.8 | Cing Gyr | 8.1 |
| Parkinson's2 | | Huntington's2 | |
| BA17 | 18.3 | Cing Gyr PSP | 8.3 |
| Huntington's | | | |
| BA17 | 0.0 | Cing Gyr PSP2 | 4.0 |
| Huntington's2 | | | |
| BA17 | 7.6 | Cing Gyr | 0.0 |
| Depression | | Depression | |
| BA17 | 3.7 | Cing Gyr | 2.9 |
| Depression2 | | Depression2 | |

AI_comprehensive panel_v1.0 Summary: Ag2897 Highest expression of the CG58567-01 gene is detected in 112427 match control psoriasis sample-F (CT=30). Furthermore, this expression is down-regulated in the corresponding psoriasis sample (CT=33). Therefore expression of this gene can be used to distinguish between these samples. In addition, the expression of this gene is up-regulated in lung from emphysema and COPD patients, which is consistent with its expression in “stressed” small airway epithelium, lung fibroblasts and lung endothelium (treated with TNF-a and IL-1). Therapeutic modulation of the expression of this putative protein and/or signaling via this protein by antibodies, small moleculesvor protein therapeutics may inhibit inflammation in lung tissue due to asthma, emphysema and other COPD type diseases

CNS_neurodegeneration_v1.0 Summary: Ag2897 This panel does not show differential expression of the CG58567-01 gene in Alzheimer's disease. However, this expression profile confirms the presence of this gene in the brain. Please see panel 1.3D for discussion of utility of this gene in the central nervous system.

Panel 1.2 Summary: Ag705 Two experiments with the same probe and primer show highest expression of the CG58567-05 gene in an ovarian cancer cell line and the cerebral cortex (CTs=25-26). This gene is also expressed in other parts of the central nervous system, including the spinal cord, amygdala, and hippocampus. Please see Panel 1.3D for further discussion of utility of this gene in the central nervous system.

In addition to expression in the ovarian cancer cell line, this gene is also expressed in a cluster of cell lines derived from ovarian and brain cancers. Thus, expression of this gene could be used to differentiate between these samples and other samples on this panel and as a marker to detect the presence of these cancers. Furthermore, therapeutic modulation of the expression or function of this gene may be effective in the treatment of ovarian and brain cancers.

Panel 1.3D Summary: Ag2897 Tow experiments with the same probe and primer show highest expression of this gene, a protocadherin homolog, in the spinal cord and the cerebral cortex (CTs=31.5). Low levels of expression are also seen in other regions of the brain including the amygdala and the hippocampus. The cadherins have been shown to be critical for CNS development, specifically for the guidance of axons, dendrites and/or growth cones in general. Therapeutic modulation of the levels of this protein, or possible signaling via this protein may be of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntingdon's, vascular dementia or any neurodegenerative disease). Since protocadherins play an important role in synaptogenesis this gene product may also be involved in depression, schizophrenia, which also involve synaptogenesis. Because this cadherin shows highest expression in the cerebellum, this is also an excellent candidate for the spinocerebellar ataxias as well.

Significant levels of expression are also seen in cell lines derived from ovarian, renal and brain cancers. Thus, expression of this gene could be used to differentiate between these samples and other samples on this panel and as a marker to detect the presence of these cancers. Furthermore, therapeutic modulation of the expression or function of this gene may be effective in the treatment of ovarian, renal and brain cancers.

References:

Hilschmann N, Barnikol H U, Barnikol-Watanabe S, Gotz H, Kratzin H, Thinnes F P. The immunoglobulin-like genetic predetermination of the brain; the protocadherins, blueprint of the neuronal network. Naturwissenschaften January 2001;88(1):2-12

Panel 2D Summary: Ag2897 Two experiments with the same probe and primer produce results that are in reasonable agreement, with highest expression of the CG58567-01 gene in ovarian and kidney cancers (CTs=30.5). Significant levels of expression are also seen in lung and uterine cancers. In addition, higher levels of expression are seen in these cancers than in the corresponding normal adjacent tissues. Thus, therapeutic targeting of this gene product with a human monoclonal antibody is anticipated to limit or block the extent of tumor cell migration and invasion, preferably in kidney, lung, uterine and ovarian tumor tumors.

Panel 3D Summary: Ag2897 Highest expression of the CG58567-01 gene is seen in a lung cancer cell line (CT=30.1). Thus, expression of this gene could be used to differentiate between this sample and other samples on this panel. Please see Panel 2D for further discussion of utility of this gene in cancer.

Panel 4D Summary: Ag2897 Two experiments with the same probe and primer produce results that are in reasonable agreement, with the CG58567-01 gene highly up-regulated in small airway epithelium and astrocytes stimulated with TNF-alpha and IL-1 beta (CTs=33-34). Other tissues in the lung also up regulate the expression of this gene including lung microvascular endothelium and lung fibroblasts in response to TNF alpha or the Th2 elaborated cytokine IL-4. This suggests that this molecule could be expressed as a result of inflammation particularly during asthma since TNFalpha and IL-4 may play important roles in the pathology of this disease. Based on the expression profile of this transcript and the types of cytokines which induce it, antibodies to CG58567-01 may inhibit inflammation in lung tissue due to asthma, emphysema and other COPD type diseases.

Panel CNS_{—}1 Summary: Ag2897 The results of this panel confirm expression of the CG58567-01 gene in the brain. Please see Panel 1.3D for discussion of utility of this gene in the central nervous system.

### W. NOV29and NOV29c (CG59243-01 and CG59243-02; MITOCHONDRIAL CARRIER PROTEIN)

Expression of gene CG59243-01 and CG59243-02 was assessed using the primer-probe set Ag3415, described in Table WA. Results of the RTQ-PCR runs are shown in Tables WB, WC, WD, and WE. Please note that CG59243-02 represents a full-length physical clone of the CG59243-01 gene, validating the prediction of the gene sequence.

TABLE WA
Probe Name Ag3415

| | | | | Start |
|---|---|---|---|---|
| Primers | Sequences | | Length | Position |
| Forward | 5′-tataaggttgtgttccggca-3′ | (SEQ ID NO:302) | 20 | 145 |
| Probe | TET-5′-gcagtgtcagaggctgtgagacagct-3′-TAMRA | (SEQ ID NO:303) | 26 | 181 |
| Reverse | 5′-tattgaggaccctcatgcca-3′ | (SEQ ID NO:304) | 20 | 208 |

TABLE WB
CNS_neurodegeneration_v1.0

| | Rel. Exp.(%) Ag3415, | | Rel. Exp.(%) Ag3415, |
|---|---|---|---|
| Tissue Name | Run 206533692 | Tissue Name | Run 206533692 |
| AD 1 Hippo | 40.6 | Control (Path) 3 | 15.5 |
| | | Temporal Ctx | |
| AD 2 Hippo | 43.2 | Control (Path) 4 | 28.3 |
| | | Temporal Ctx | |
| AD 3 Hippo | 13.2 | AD 1 Occipital Ctx | 17.2 |
| AD 4 Hippo | 13.9 | AD 2 Occipital Ctx | 0.0 |
| | | (Missing) | |
| AD 5 hippo | 79.0 | AD 3 Occipital Ctx | 7.2 |
| AD 6 Hippo | 40.6 | AD 4 Occipital Ctx | 21.9 |
| Control 2 Hippo | 18.0 | AD 5 Occipital Ctx | 4.7 |
| Control 4 Hippo | 36.6 | AD 6 Occipital Ctx | 25.3 |
| Control (Path) 3 | 8.4 | Control 1 Occipital | 8.0 |
| Hippo | | Ctx | |
| AD 1 Temporal Ctx | 28.9 | Control 2 Occipital | 48.6 |
| | | Ctx | |
| AD 2 Temporal Ctx | 42.0 | Control 3 Occipital | 11.0 |
| | | Ctx | |
| AD 3 Temporal Ctx | 13.8 | Control 4 Occipital | 16.5 |
| | | Ctx | |
| AD 4 Temporal Ctx | 22.5 | Control (Path) 1 | 100.0 |
| | | Occipital Ctx | |
| AD 5 Inf Temporal | 73.7 | Control (Path) 2 | 14.0 |
| Ctx | | Occipital Ctx | |
| AD 5 Sup Temporal | 65.5 | Control (Path) 3 | 5.8 |
| Ctx | | Occipital Ctx | |
| AD 6 Inf Temporal | 27.0 | Control (Path) 4 | 24.3 |
| Ctx | | Occipital Ctx | |
| AD 6 Sup Temporal | 20.0 | Control 1 Parietal | 21.0 |
| Ctx | | Ctx | |
| Control 1 Temporal | 18.7 | Control 2 Parietal | 49.0 |
| Ctx | | Ctx | |
| Control 2 Temporal | 27.9 | Control 3 Parietal | 9.0 |
| Ctx | | Ctx | |
| Control 3 Temporal | 9.3 | Control (Path) 1 | 55.9 |
| Ctx | | Parietal Ctx | |
| Control 4 Temporal | 17.4 | Control (Path) 2 | 31.4 |
| Ctx | | Parietal Ctx | |
| Control (Path) 1 | 34.9 | Control (Path) 3 | 4.6 |
| Temporal Ctx | | Parietal Ctx | |
| Control (Path) 2 | 33.2 | Control (Path) 4 | 44.4 |
| Temporal Ctx | | Parietal Ctx | |

TABLE WC
Panel 1.3D

| | Rel. Exp.(%) Ag3415, | | Rel. Exp.(%) Ag3415, |
|---|---|---|---|
| Tissue Name | Run 167617385 | Tissue Name | Run 167617385 |
| Liver adenocarcinoma | 0.0 | Kidney (fetal) | 19.8 |
| Pancreas | 1.1 | Renal ca. 786-0 | 13.7 |
| Pancreatic ca. CAPAN 2 | 0.0 | Renal ca. A498 | 2.6 |
| Adrenal gland | 0.9 | Renal ca. RXF 393 | 9.1 |
| Thyroid | 0.0 | Renal ca. ACHN | 13.5 |
| Salivary gland | 0.0 | Renal ca. UO-31 | 20.6 |
| Pituitary gland | 5.6 | Renal ca. TK-10 | 32.8 |
| Brain (fetal) | 55.1 | Liver | 4.5 |
| Brain (whole) | 20.0 | Liver (fetal) | 0.0 |
| Brain (amygdala) | 23.2 | Liver ca. | 2.3 |
| | | (hepatoblast) HepG2 | |
| Brain (cerebellum) | 17.3 | Lung | 5.9 |
| Brain (hippocampus) | 12.5 | Lung (fetal) | 4.8 |
| Brain (substantia nigra) | 26.2 | Lung ca. (small cell) | 2.8 |
| | | LX-1 | |
| Brain (thalamus) | 23.8 | Lung ca. (small cell) | 2.7 |
| | | NCI-H69 | |
| Cerebral Cortex | 32.5 | Lung ca. (s.cell var.) | 89.5 |
| | | SHP-77 | |
| Spinal cord | 20.2 | Lung ca. (large | 2.6 |
| | | cell) NCI-H460 | |
| glio/astro U87-MG | 0.0 | Lung ca. (non-sm. | 0.0 |
| | | cell) A549 | |
| glio/astro U-118-MG | 2.1 | Lung ca. (non-s.cell) | 3.7 |
| | | NCI-H23 | |
| astrocytoma SW1783 | 15.9 | Lung ca. (non-s.cell) | 8.2 |
| | | HOP-62 | |
| neuro*; met SK-N-AS | 8.3 | Lung ca. (non-s.cl) | 17.3 |
| | | NCI-H522 | |
| astrocytoma SF-539 | 2.3 | Lung ca. (squam.) | 6.4 |
| | | SW 900 | |
| astrocytoma SNB-75 | 6.7 | Lung ca. (squam.) | 18.8 |
| | | NCI-H596 | |
| glioma SNB-19 | 0.0 | Mammary gland | 1.1 |
| glioma U251 | 23.5 | Breast ca.* (pl.ef) | 2.2 |
| | | MCF-7 | |
| glioma SF-295 | 0.0 | Breast ca.* (pl.ef) | 2.6 |
| | | MDA-MB-231 | |
| Heart (fetal) | 10.0 | Breast ca.* (pl.ef) | 1.3 |
| | | T47D | |
| Heart | 4.6 | Breast ca. BT-549 | 7.7 |
| Skeletal muscle (fetal) | 2.0 | Breast ca. MDA-N | 12.2 |
| Skeletal muscle | 3.4 | Ovary | 4.2 |
| Bone marrow | 3.8 | Ovarian ca. OVCAR-3 | 17.7 |
| Thymus | 5.4 | Ovarian ca. OVCAR-4 | 0.0 |
| Spleen | 24.8 | Ovarian ca. OVCAR-5 | 0.0 |
| Lymph node | 43.5 | Ovarian ca. OVCAR-8 | 3.5 |
| Colorectal | 6.0 | Ovarian ca. IGROV-1 | 2.4 |
| Stomach | 6.8 | Ovarian ca.* (ascites) | 3.5 |
| | | SK-OV-3 | |
| Small intestine | 9.3 | Uterus | 4.4 |
| Colon ca. SW480 | 6.5 | Placenta | 1.9 |
| Colon ca.* | 100.0 | Prostate | 0.0 |
| SW620 (SW480 met) | | | |
| Colon ca. HT29 | 0.0 | Prostate ca.* (bone | 0.0 |
| | | met) PC-3 | |
| Colon ca. HCT-116 | 0.0 | Testis | 14.4 |
| Colon ca. CaCo-2 | 3.8 | Melanoma | 0.0 |
| | | Hs688(A).T | |
| Colon ca. | 0.0 | Melanoma* (met) | 0.0 |
| tissue(ODO3866) | | Hs688(B).T | |
| Colon ca. HCC-2998 | 0.0 | Melanoma UACC-62 | 5.1 |
| Gastric ca.* (liver met) | 0.0 | Melanoma M14 | 0.0 |
| NCI-N87 | | | |
| Bladder | 6.4 | Melanoma LOX | 0.0 |
| | | IMVI | |
| Trachea | 0.0 | Melanoma* (met) | 2.1 |
| | | SK-MEL-5 | |
| Kidney | 0.0 | Adipose | 3.3 |

TABLE WD
Panel 2D

| | Rel. Exp.(%) | | Rel. Exp.(%) |
|---|---|---|---|
| | Ag3415, Run | | Ag3415, Run |
| Tissue Name | 169596730 | Tissue Name | 169596730 |
| Normal Colon | 39.0 | Kidney Margin | 0.0 |
| | | 8120608 | |
| CC Well to Mod Diff | 8.7 | Kidney Cancer | 7.7 |
| (ODO3866) | | 8120613 | |
| CC Margin (ODO3866) | 0.0 | Kidney Margin | 0.0 |
| | | 8120614 | |
| CC Gr.2 rectosigmoid | 11.7 | Kidney Cancer | 7.7 |
| (ODO3868) | | 9010320 | |
| CC Margin (ODO3868) | 0.8 | Kidney Margin | 0.0 |
| | | 9010321 | |
| CC Mod Diff (ODO3920) | 7.6 | Normal Uterus | 2.0 |
| CC Margin (ODO3920) | 3.6 | Uterus Cancer 064011 | 23.3 |
| CC Gr.2 ascend colon | 19.9 | Normal Thyroid | 2.0 |
| (ODO3921) | | | |
| CC Margin (ODO3921) | 10.2 | Thyroid Cancer | 4.4 |
| | | 064010 | |
| CC from Partial | 1.9 | Thyroid Cancer | 2.3 |
| Hepatectomy (ODO4309) | | A302152 | |
| Mets | | | |
| Liver Margin (ODO4309) | 3.1 | Thyroid Margin | 5.9 |
| | | A302153 | |
| Colon mets to lung | 3.6 | Normal Breast | 19.6 |
| (OD04451-01) | | | |
| Lung Margin (OD04451- | 2.5 | Breast Cancer | 5.3 |
| 02) | | (OD04566) | |
| Normal Prostate 6546-1 | 1.1 | Breast Cancer | 16.5 |
| | | (OD04590-01) | |
| Prostate Cancer | 14.5 | Breast Cancer Mets | 29.5 |
| (OD04410) | | (OD04590-03) | |
| Prostate Margin | 17.0 | Breast Cancer | 12.9 |
| (OD04610) | | Metastasis (OD04655-05) | |
| Prostate Cancer | 10.6 | Breast Cancer 064006 | 13.6 |
| (OD04720-01) | | | |
| Prostate Margin | 16.3 | Breast Cancer 1024 | 7.4 |
| (OD04720-02) | | | |
| Normal Lung 061010 | 60.3 | Breast Cancer | 10.1 |
| | | 9100266 | |
| Lung Met to Muscle | 1.8 | Breast Margin | 5.0 |
| (ODO4286) | | 9100265 | |
| Muscle Margin | 2.0 | Breast Cancer | 31.9 |
| (ODO4286) | | A209073 | |
| Lung Malignant Cancer | 7.7 | Breast Margin | 10.5 |
| (OD03126) | | A209073 | |
| Lung Margin (OD03126) | 12.6 | Normal Liver | 4.3 |
| Lung Cancer (OD04404) | 15.5 | Liver Cancer 064003 | 0.0 |
| Lung Margin (OD04404) | 7.6 | Liver Cancer 1025 | 1.9 |
| Lung Cancer (OD04565) | 1.8 | Liver Cancer 1026 | 1.9 |
| Lung Margin (OD04565) | 22.5 | Liver Cancer 6004-T | 4.9 |
| Lung Cancer (OD04237- | 100.0 | Liver Tissue 6004-N | 6.6 |
| 01) | | | |
| Lung Margin (OD04237- | 7.2 | Liver Cancer 6005-T | 14.7 |
| 02) | | | |
| Ocular Mel Met to Liver | 0.0 | Liver Tissue 6005-N | 5.3 |
| (ODO4310) | | | |
| Liver Margin (ODO4310) | 3.6 | Normal Bladder | 34.2 |
| Melanoma Mets to Lung | 3.6 | Bladder Cancer 1023 | 2.1 |
| (OD04321) | | | |
| Lung Margin (OD04321) | 14.7 | Bladder Cancer | 23.5 |
| | | A302173 | |
| Normal Kidney | 4.7 | Bladder Cancer | 17.1 |
| | | (OD04718-01) | |
| Kidney Ca, Nuclear grade | 19.9 | Bladder Normal | 4.1 |
| 2 (OD04338) | | Adjacent (OD04718- | |
| | | 03) | |
| Kidney Margin | 3.2 | Normal Ovary | 3.4 |
| (OD04338) | | | |
| Kidney Ca Nuclear grade | 25.2 | Ovarian Cancer | 14.2 |
| 1/2 (OD04339) | | 064008 | |
| Kidney Margin | 2.2 | Ovarian Cancer | 4.1 |
| (OD04339) | | (OD04768-07) | |
| Kidney Ca, Clear cell type | 6.1 | Ovary Margin | 8.2 |
| (OD04340) | | (OD04768-08) | |
| Kidney Margin | 0.0 | Normal Stomach | 29.3 |
| (OD04340) | | | |
| Kidney Ca, Nuclear grade | 0.0 | Gastric Cancer | 9.9 |
| 3 (OD04348) | | 9060358 | |
| Kidney Margin | 11.4 | Stomach Margin | 10.7 |
| (OD04348) | | 9060359 | |
| Kidney Cancer | 23.8 | Gastric Cancer | 16.4 |
| (OD04622-01) | | 9060395 | |
| Kidney Margin | 7.0 | Stomach Margin | 17.0 |
| (OD04622-03) | | 9060394 | |
| Kidney Cancer | 15.9 | Gastric Cancer | 5.1 |
| (OD04450-01) | | 9060397 | |
| Kidney Margin | 1.7 | Stomach Margin | 7.7 |
| (OD04450-03) | | 9060396 | |
| Kidney Cancer 8120607 | 4.4 | Gastric Cancer 064005 | 34.6 |

TABLE WE
Panel 4D

| | Rel. Exp.(%) | | Rel. Exp.(%) |
|---|---|---|---|
| | Ag3415, Run | | Ag3415, Run |
| Tissue Name | 166385451 | Tissue Name | 166385451 |
| Secondary Th1 act | 1.4 | HUVEC IL-1 beta | 1.5 |
| Secondary Th2 act | 0.4 | HUVEC IFN gamma | 1.7 |
| Secondary Tr1 act | 3.1 | HUVEC TNF alpha + IFN | 1.3 |
| | | gamma | |
| Secondary Th1 rest | 1.6 | HUVEC TNF alpha + IL4 | 4.2 |
| Secondary Th2 rest | 0.8 | HUVEC IL-11 | 1.6 |
| Secondary Tr1 rest | 0.5 | Lung Microvascular EC | 3.3 |
| | | none | |
| Primary Th1 act | 3.6 | Lung Microvascular EC | 4.7 |
| | | TNF alpha + IL-1 beta | |
| Primary Th2 act | 2.0 | Microvascular Dermal EC | 1.3 |
| | | none | |
| Primary Tr1 act | 0.9 | Microvascular Dermal EC | 1.1 |
| | | TNF alpha + IL-1 beta | |
| Primary Th1 rest | 3.9 | Bronchial epithelium | 0.0 |
| | | TNF alpha + IL-1 beta | |
| Primary Th2 rest | 1.5 | Small airway epithelium | 0.0 |
| | | none | |
| Primary Tr1 rest | 0.9 | Small airway epithelium | 0.2 |
| | | TNF alpha + IL-1 beta | |
| CD45RA CD4 | 2.6 | Coronery artery SMC rest | 0.1 |
| lymphocyte act | | | |
| CD45RO CD4 | 8.5 | Coronery artery SMC | 0.0 |
| lymphocyte act | | TNF alpha + IL-1 beta | |
| CD8 lymphocyte act | 17.1 | Astrocytes rest | 3.1 |
| Secondary CD8 | 4.9 | Astrocytes TNF alpha + IL- | 0.5 |
| lymphocyte rest | | 1 beta | |
| Secondary CD8 | 5.9 | KU-812 (Basophil) rest | 1.2 |
| lymphocyte act | | | |
| CD4 lymphocyte none | 2.1 | KU-812 (Basophil) | 4.3 |
| | | PMA/ionomycin | |
| 2ry Th1/Th2/Tr1_anti- | 1.9 | CCD1106 (Keratinocytes) | 2.2 |
| CD95 CH11 | | none | |
| LAK cells rest | 7.9 | CCD1106 (Keratinocytes) | 0.8 |
| | | TNFa1pha + IL-1 beta | |
| LAK cells IL-2 | 30.8 | Liver cirrhosis | 2.7 |
| LAK cells IL-2 + IL-12 | 6.0 | Lupus kidney | 0.0 |
| LAK cells IL-2 + IFN | 7.4 | NCI-H292 none | 0.3 |
| gamma | | | |
| LAK cells IL-2 + IL-18 | 11.7 | NCI-H292 IL-4 | 0.0 |
| LAK cells | 1.1 | NCI-H292 IL-9 | 0.0 |
| PMA/ionomycin | | | |
| NK Cells IL-2 rest | 19.8 | NCI-H292 IL-13 | 0.0 |
| Two Way MLR 3 day | 9.5 | NCI-H292 IFN gamma | 0.7 |
| Two Way MLR 5 day | 4.1 | HPAEC none | 1.5 |
| Two Way MLR 7 day | 12.5 | HPAEC TNF alpha + IL-1 | 0.5 |
| | | beta | |
| PBMC rest | 4.1 | Lung fibroblast none | 0.0 |
| PBMC PWM | 28.3 | Lung fibroblast TNF | 0.2 |
| | | alpha + IL-1 beta | |
| PBMC PHA-L | 10.8 | Lung fibroblast IL-4 | 2.1 |
| Ramos (B cell) none | 34.6 | Lung fibroblast IL-9 | 0.5 |
| Ramos (B cell) | 100.0 | Lung fibroblast IL-13 | 0.8 |
| ionomycin | | | |
| B lymphocytes PWM | 32.3 | Lung fibroblast IFN | 0.8 |
| | | gamma | |
| B lymphocytes CD40L | 7.0 | Dermal fibroblast | 0.7 |
| and IL-4 | | CCD1070 rest | |
| EOL-1 dbcAMP | 0.0 | Dermal fibroblast | 28.7 |
| | | CCD1070 TNF alpha | |
| EOL-1 dbcAMP | 0.5 | Dermal fibroblast | 0.0 |
| PMA/ionomycin | | CCD1070 IL-1 beta | |
| Dendritic cells none | 0.4 | Dermal fibroblast IFN | 0.0 |
| | | gamma | |
| Dendritic cells LPS | 0.2 | Dermal fibroblast IL-4 | 0.3 |
| Dendritic cells anti- | 0.3 | IBD Colitis 2 | 0.7 |
| CD40 | | | |
| Monocytes rest | 0.2 | IBD Crohn's | 0.0 |
| Monocytes LPS | 1.6 | Colon | 4.0 |
| Macrophages rest | 0.5 | Lung | 1.1 |
| Macrophages LPS | 0.2 | Thymus | 0.7 |
| HUVEC none | 2.1 | Kidney | 3.0 |
| HUVEC starved | 3.1 | | |

AI_comprehensive panel_v1.0 Summary: Ag3415 Results from one experiment with the CG59243-01 gene are not included. The amp plot indicates that there were experimental difficulties with this run.

CNS_neurodegeneration_v1.0 Summary: Ag3415 This panel confirms the expression of the CG59243-01 gene at low levels in the brains of an independent group of individuals. However, no differential expression of this gene was detected between Alzheimer's disease postmortem brains and those of non-demented controls in this experiment. Please see Panel 1.3D for a discussion of the potential utility of this gene in treatment of central nervous system disorders.

Panel 1.3D Summary: Ag3415 Highest expression of the CG59243-01 gene is seen in a colon cancer cell line (CT=33.2), with significant expression also seen in a lung cancer cell line. Thus, expression of this gene could be used to differentiate between these samples and other samples on this panel and as a marker to detect the presence of colon or lung cancer. Furthermore, therapeutic modulation of the expression or function of this gene may be effective in the treatment of colon or lung cancer.

In addition, this gene is expressed at low levels in fetal brain and cerebral cortex. Therefore, this gene may play a role in central nervous system development and CNS disorders such as Alzheimer's disease, Parkinson's disease, seizures, epilepsy, multiple sclerosis, schizophrenia and depression.

Panel 2D Summary: Ag3415 Highest expression of the CG59243-01 gene is seen in a lung cancer (CT=32.5), consistent with expression in Panel 1.3D. Thus, expression of this gene could be used to differentiate between this sample and other samples on this panel. Please see Panel 1.3D for a discussion of the potential utility of this gene in treatment of cancer.

Panel 4D Summary: Ag3415 Highest expression of the CG59243-01 gene is seen in the B cell line Ramos, treated with ionomycin (CT=29.5). Low, but significant levels of expression of this transcript are also seen in activated B cells (B cells treated with PWM or CD40L+IL4). This transcript is also expressed in PBMC treated with the B cell mitogen, PWM, confirming the importance of this gene's expression in activated B cells. B cells represent a principle component of immunity and contribute to the immune response in a number of important functional roles, including antibody production. For example, production of antibodies against self-antigens is a major component in an autoimmune disorders such a systemic lupus erythematosus. Since B cells play an important role in autoimmunity, inflammatory processes and inflammatory cascades, therapeutic modulation of this gene product may reduce or eliminate the symptoms or patients suffering from asthma, allergies, chronic obstructive pulmonary disease, emphysema, Crohn's disease, ulcerative colitis, rheumatoid arthritis, psoriasis, osteoarthritis, and other autoimmune disorders including systemic lupus erythematosus.

### X. NOV30a (CG59534-01: MEMBRANE GLYCOPROTEIN)

Expression of gene CG59534-01 was assessed using the primer-probe sets Ag5041 and Ag5043, described in Tables XA and XB. Results of the RTQ-PCR runs are shown in Tables XC and XD.

TABLE XA
Probe Name Ag5041

| | | | | Start |
|---|---|---|---|---|
| Primers | Sequences | | Length | Position |
| Forward | 5′-ggtgttggtacctgtggattt-3′ | (SEQ ID NO:305) | 21 | 4154 |
| Probe | TET-5′-tctatactgatgttttcgttttgccaa-3′-TAMRA | (SEQ ID NO:306) | 27 | 4176 |
| Reverse | 5′-cccaaggccaatgtaatactc-3′ | (SEQ ID NO:307) | 21 | 4209 |

TABLE XB
Probe Name Ag5043

| | | | | Start |
|---|---|---|---|---|
| Primers | Sequences | | Length | Position |
| Forward | 5′-cacaagcaaaaggtcagaaca-3′ | (SEQ ID NO:308) | 21 | 4024 |
| Probe | TET-5′-aaacactgcctttcctcctcctctta-3′-TAMRA | (SEQ ID NO:309) | 26 | 4048 |
| Reverse | 5′-ggccacatttgcttttatcata-3′ | (SEQ ID NO:310) | 22 | 4077 |

TABLE XC
General_screening_panel_v1.5

| | Rel. Exp.(%) | Rel. Exp.(%) | | Rel. Exp.(%) | Rel. Exp.(%) |
|---|---|---|---|---|---|
| | Ag5041, Run | Ag5043, Run | | Ag5041, Run | Ag5043, Run |
| Tissue Name | 228967328 | 228969275 | Tissue Name | 228967328 | 228969275 |
| Adipose | 1.1 | 4.3 | Renal ca. TK-10 | 2.4 | 10.3 |
| Melanoma* | 0.8 | 4.2 | Bladder | 3.7 | 15.2 |
| Hs688(A).T | | | | | |
| Melanoma* | 1.1 | 2.7 | Gastric ca. (liver | 100.0 | 5.5 |
| Hs688(B).T | | | met.) NCI-N87 | | |
| Melanoma* | 0.6 | 2.6 | Gastric ca. KATO | 13.8 | 77.9 |
| M14 | | | III | | |
| Melanoma* | 2.4 | 7.7 | Colon ca. SW- | 1.4 | 4.0 |
| LOXIMVI | | | 948 | | |
| Melanoma* | 1.0 | 5.5 | Colon ca. SW480 | 3.0 | 12.2 |
| SK-MEL-5 | | | | | |
| Squamous cell | 0.7 | 3.0 | Colon ca.* | 5.3 | 23.0 |
| carcinoma | | | (SW480 met) | | |
| SCC-4 | | | SW620 | | |
| Testis Pool | 2.1 | 5.9 | Colon ca. HT29 | 1.3 | 5.5 |
| Prostate ca.* | 0.3 | 1.3 | Colon ca. HCT- | 0.9 | 4.0 |
| (bone met) | | | 116 | | |
| PC-3 | | | | | |
| Prostate Pool | 4.6 | 11.0 | Colon ca. CaCo-2 | 5.4 | 25.7 |
| Placenta | 0.2 | 1.0 | Colon cancer | 2.2 | 9.3 |
| | | | tissue | | |
| Uterus Pool | 2.7 | 3.5 | Colon ca. | 0.7 | 2.2 |
| | | | SW1116 | | |
| Ovarian ca. | 3.0 | 13.7 | Colon ca. Colo- | 1.3 | 3.6 |
| OVCAR-3 | | | 205 | | |
| Ovarian ca. | 1.6 | 7.3 | Colon ca. SW-48 | 1.3 | 3.5 |
| SK-OV-3 | | | | | |
| Ovarian ca. | 0.9 | 2.9 | Colon Pool | 3.4 | 0.6 |
| OVCAR-4 | | | | | |
| Ovarian ca. | 10.7 | 65.5 | Small Intestine | 3.4 | 15.6 |
| OVCAR-5 | | | Pool | | |
| Ovarian ca. | 11.8 | 71.7 | Stomach Pool | 3.6 | 11.7 |
| IGROV-1 | | | | | |
| Ovarian ca. | 5.0 | 25.3 | Bone Marrow | 2.4 | 5.2 |
| OVCAR-8 | | | Pool | | |
| Ovary | 1.1 | 6.2 | Fetal Heart | 2.6 | 4.4 |
| Breast ca. | 3.6 | 20.0 | Heart Pool | 1.7 | 3.0 |
| MCF-7 | | | | | |
| Breast ca. | 2.3 | 9.7 | Lymph Node | 6.9 | 24.1 |
| MDA-MB- | | | Pool | | |
| 231 | | | | | |
| Breast ca. BT | 0.8 | 2.0 | Fetal Skeletal | 2.1 | 10.3 |
| 549 | | | Muscle | | |
| Breast ca. | 10.3 | 24.8 | Skeletal Muscle | 8.3 | 28.3 |
| T47D | | | Pool | | |
| Breast ca. | 1.6 | 6.0 | Spleen Pool | 0.8 | 2.1 |
| MDA-N | | | | | |
| Breast Pool | 4.3 | 22.8 | Thymus Pool | 3.5 | 12.5 |
| Trachea | 2.5 | 11.3 | CNS cancer | 7.4 | 36.1 |
| | | | (glio/astro) U87- | | |
| | | | MG | | |
| Lung | 1.9 | 8.2 | CNS cancer | 17.0 | 100.0 |
| | | | (glio/astro) U- | | |
| | | | 118-MG | | |
| Fetal Lung | 5.6 | 29.1 | CNS cancer | 1.2 | 5.1 |
| | | | (neuro;met) SK- | | |
| | | | N-AS | | |
| Lung ca. NCI- | 0.8 | 3.8 | CNS cancer | 3.3 | 13.5 |
| N417 | | | (astro) SF-539 | | |
| Lung ca. LX-1 | 2.8 | 11.1 | CNS cancer | 7.1 | 34.4 |
| | | | (astro) SNB-75 | | |
| Lung ca. NCI- | 0.6 | 2.8 | CNS cancer (glio) | 14.0 | 80.1 |
| H146 | | | SNB-19 | | |
| Lung ca. SHP- | 3.6 | 20.9 | CNS cancer (glio) | 2.9 | 11.9 |
| 77 | | | SF-295 | | |
| Lung ca. A549 | 1.1 | 2.8 | Brain (Amygdala) | 2.7 | 10.6 |
| | | | Pool | | |
| Lung ca. NCI- | 0.6 | 2.4 | Brain | 7.8 | 50.0 |
| H526 | | | (cerebellum) | | |
| Lung ca. NCI- | 1.6 | 5.6 | Brain (fetal) | 2.8 | 16.8 |
| H23 | | | | | |
| Lung ca. NCI- | 1.2 | 2.8 | Brain | 4.9 | 20.2 |
| H460 | | | (Hippocampus) | | |
| | | | Pool | | |
| Lung ca. | 1.2 | 4.3 | Cerebral Cortex | 5.0 | 18.8 |
| HOP-62 | | | Pool | | |
| Lung ca. NCI- | 0.4 | 1.3 | Brain (Substantia | 3.0 | 11.0 |
| H522 | | | nigra) Pool | | |
| Liver | 0.1 | 0.4 | Brain (Thalamus) | 6.5 | 24.1 |
| | | | Pool | | |
| Fetal Liver | 0.7 | 3.1 | Brain (whole) | 2.4 | 11.3 |
| Liver ca. | 2.8 | 12.1 | Spinal Cord Pool | 4.2 | 14.1 |
| HepG2 | | | | | |
| Kidney Pool | 5.0 | 21.6 | Adrenal Gland | 0.6 | 2.2 |
| Fetal Kidney | 1.5 | 4.8 | Pituitary gland | 1.5 | 5.1 |
| | | | Pool | | |
| Renal ca. 786-0 | 0.4 | 1.9 | Salivary Gland | 0.7 | 2.1 |
| Renal ca. | 0.3 | 0.8 | Thyroid (female) | 0.9 | 3.3 |
| A498 | | | | | |
| Renal ca. | 0.4 | 1.8 | Pancreatic ca. | 0.5 | 1.5 |
| ACHN | | | CAPAN2 | | |
| Renal ca. UO- | 1.8 | 8.5 | Pancreas Pool | 5.8 | 28.9 |
| 31 | | | | | |

TABLE XD
Panel 4.1D

| | Rel. | Rel. | | Rel. | Rel. |
|---|---|---|---|---|---|
| | Exp.(%) | Exp.(%) | | Exp.(%) | Exp.(%) |
| | Ag5041, | Ag5043, | | Ag5041, | Ag5043, |
| | Run | Run | | Run | Run |
| Tissue Name | 223784808 | 223785123 | Tissue Name | 223784808 | 223785123 |
| Secondary Th1 act | 37.4 | 15.3 | HUVEC IL-1 beta | 37.6 | 28.5 |
| Secondary Th2 act | 25.3 | 9.6 | HUVEC IFN | 31.6 | 9.0 |
| | | | gamma | | |
| Secondary Tr1 act | 33.0 | 14.1 | HUVEC TNF | 19.6 | 10.2 |
| | | | alpha + IFN | | |
| | | | gamma | | |
| Secondary Th1 rest | 14.7 | 8.2 | HUVEC TNF | 29.3 | 19.2 |
| | | | alpha + IL4 | | |
| Secondary Th2 rest | 13.5 | 10.5 | HUVEC IL-11 | 13.5 | 7.5 |
| Secondary Tr1 rest | 10.8 | 8.1 | Lung | 28.5 | 11.7 |
| | | | Microvascular EC | | |
| | | | none | | |
| Primary Th1 act | 33.7 | 20.2 | Lung | 28.7 | 16.7 |
| | | | Microvascular EC | | |
| | | | TNF alpha + IL- | | |
| | | | 1 beta | | |
| Primary Th2 act | 47.6 | 19.3 | Microvascular | 9.6 | 6.2 |
| | | | Dermal EC none | | |
| Primary Tr1 act | 32.5 | 19.2 | Microsvasular | 23.8 | 11.4 |
| | | | Dermal EC | | |
| | | | TNF alpha + IL- | | |
| | | | 1 beta | | |
| Primary Th1 rest | 15.9 | 5.2 | Bronchial | 11.4 | 5.6 |
| | | | epithelium | | |
| | | | TNF alpha + | | |
| | | | IL1 beta | | |
| Primary Th2 rest | 13.0 | 6.6 | Small airway | 3.6 | 2.3 |
| | | | epithelium none | | |
| Primary Tr1 rest | 17.7 | 11.0 | Small airway | 4.1 | 3.7 |
| | | | epithelium | | |
| | | | TNF alpha + IL- | | |
| | | | 1 beta | | |
| CD45RA CD4 | 40.1 | 11.0 | Coronery artery | 12.8 | 6.0 |
| lymphocyte act | | | SMC rest | | |
| CD45RO CD4 | 30.6 | 15.4 | Coronery artery | 22.4 | 11.0 |
| lymphocyte act | | | SMC TNF alpha + | | |
| | | | IL-1 beta | | |
| CD8 lymphocyte | 28.9 | 16.3 | Astrocytes rest | 17.4 | 8.3 |
| act | | | | | |
| Secondary CD8 | 29.3 | 17.6 | Astrocytes | 37.1 | 38.7 |
| lymphocyte rest | | | TNF alpha + IL- | | |
| | | | 1 beta | | |
| Secondary CD8 | 18.7 | 9.9 | KU-812 (Basophil) | 9.0 | 4.5 |
| lymphocyte act | | | rest | | |
| CD4 lymphocyte | 33.0 | 12.3 | KU-812 (Basophil) | 9.5 | 6.6 |
| none | | | PMA/ionomycin | | |
| 2ry | 33.7 | 13.8 | CCD1106 | 16.7 | 10.6 |
| Th1/Th2/Tr1_anti- | | | (Keratinocytes) | | |
| CD95 CH11 | | | none | | |
| LAK cells rest | 24.7 | 10.1 | CCD1106 | 7.4 | 4.0 |
| | | | (Keratinocytes) | | |
| | | | TNF alpha + IL- | | |
| | | | 1 beta | | |
| LAK cells IL-2 | 26.1 | 8.4 | Liver cirrhosis | 10.4 | 3.9 |
| LAK cells IL-2 + | 28.1 | 11.3 | NCI-H292 none | 8.1 | 4.9 |
| IL-12 | | | | | |
| LAK cells IL- | 28.9 | 14.6 | NCI-H292 IL-4 | 6.7 | 4.0 |
| 2 + IFN gamma | | | | | |
| LAK cells IL-2 + | 30.4 | 14.9 | NCI-H292 IL-9 | 7.2 | 4.0 |
| IL-18 | | | | | |
| LAK cells | 36.6 | 21.9 | NCI-H292 IL-13 | 10.6 | 4.7 |
| PMA/ionomycin | | | | | |
| NK Cells IL-2 rest | 28.7 | 17.8 | NCI-H292 IFN | 7.1 | 2.1 |
| | | | gamma | | |
| Two Way MLR 3 | 26.4 | 10.5 | HPAEC none | 21.9 | 11.6 |
| day | | | | | |
| Two Way MLR 5 | 22.5 | 9.2 | HPAEC TNF | 81.2 | 37.1 |
| day | | | alpha + IL-1 beta | | |
| Two Way MLR 7 | 16.0 | 15.8 | Lung fibroblast | 46.3 | 24.3 |
| day | | | none | | |
| PBMC rest | 11.2 | 5.6 | Lung fibroblast | 73.7 | 29.5 |
| | | | TNF alpha + IL-1 | | |
| | | | beta | | |
| PBMC PWM | 31.6 | 14.0 | Lung fibroblast IL-4 | 49.0 | 22.1 |
| PBMC PHA-L | 20.2 | 9.8 | Lung fibroblast IL-9 | 39.5 | 100.0 |
| Ramos (B cell) | 78.5 | 49.7 | Lung fibroblast IL- | 54.7 | 28.1 |
| none | | | 13 | | |
| Ramos (B cell) | 100.0 | 53.6 | Lung fibroblast | 55.5 | 36.1 |
| ionomycin | | | IFN gamma | | |
| B lymphocytes | 20.6 | 6.8 | Dermal fibroblast | 15.3 | 8.6 |
| PWM | | | CCD1070 rest | | |
| B lymphocytes | 22.1 | 8.7 | Dermal fibroblast | 33.4 | 25.5 |
| CD40L and IL-4 | | | CCD1070 TNF | | |
| | | | alpha | | |
| EOL-1 dbcAMP | 3.8 | 1.3 | Dermal fibroblast | 57.4 | 27.4 |
| | | | CCD1070 IL-1 | | |
| | | | beta | | |
| EOL-1 dbcAMP | 3.2 | 1.1 | Dermal fibroblast | 25.9 | 14.3 |
| PMA/ionomycin | | | IFN gamma | | |
| Dendritic cells none | 4.5 | 1.9 | Dermal fibroblast | 29.3 | 9.0 |
| | | | IL-4 | | |
| Dendritic cells LPS | 5.5 | 2.8 | Dermal Fibroblasts | 10.4 | 4.9 |
| | | | rest | | |
| Dendritic cells anti- | 3.3 | 1.7 | Neutrophils | 3.5 | 1.2 |
| CD40 | | | TNFa + LPS | | |
| Monocytes rest | 6.3 | 3.5 | Neutrophils rest | 3.8 | 2.2 |
| Monocytes LPS | 6.4 | 4.3 | Colon | 12.2 | 8.5 |
| Macrophages rest | 4.8 | 2.8 | Lung | 11.2 | 8.2 |
| Macrophages LPS | 1.1 | 0.9 | Thymus | 14.6 | 8.4 |
| HUVEC none | 15.9 | 9.2 | Kidney | 33.4 | 25.5 |
| HUVEC starved | 28.5 | 19.1 | | | |

General_screening_panel_v1.5 Summary: Ag5041 l/Ag5043 Two experiments with two different probe and primer sets show highest expression of the CG59534-01 gene, a putative membrane glycoprotein, in cell lines derived from brain cancer and gastric cancer (CTs=24-29). Significant expression of this gene is also seen in cell lines derived from brain cancer, ovarian cancer, and gastric cancer. Thus, expression of this gene could be used to differentiate between these samples and other samples on this panel and as a marker to detect the presence of these cancers. Furthermore, therapeutic modulation of the expression or function of this gene may be effective in the treatment of colon, ovarian, and brain cancers.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

In addition, this molecule novel membrane glycoprotein is expressed at moderate to low levels in the CNS and may be a small molecule target for the treatment of neurologic diseases such as Alzheimer's disease, Parkinson's disease, epilepsy, multiple sclerosis, schizophrenia and depression.

Panel 4.1D Summary: Ag5041/Ag5043 Two experiments with two different probe and primer sets show highest expression of the CG59534-01 gene, a putative membrane glycoprotein, in IL-9 treated fibroblasts and the B cell line Ramos treated with ionomycin (CTs=29-30). This gene is also gene is expressed at moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.5 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

### Y. NOV31a and NOV31b (CG59289-01 and CG59289-02: CRUMBS LIKE)

Expression of gene CG59289-01 and CG59289-02 was assessed using the primer-probe sets Ag3530 and Ag1932, described in Tables YA and YB. Results of the RTQ-PCR runs are shown in Tables YC, and YD.

TABLE YA
Probe Name Ag3530

| | | | | Start |
|---|---|---|---|---|
| Primers | Sequences | | Length | Position |
| Forward | 5′-gtgctgggatttcacagtacac-3′ | (SEQ ID NO:311) | 22 | 123 |
| Probe | TET-5′-tgctacttgcctaggcaaagtcacgt-3′-TAMRA | (SEQ ID NO:312) | 26 | 147 |
| Reverse | 5′-ccagtctgtcctcgttttgtag-3′ | (SEQ ID NO: 313) | 22 | 186 |

TABLE YB
Probe Name Ag1932

| | | | | Start |
|---|---|---|---|---|
| Primers | Sequences | | Length | Position |
| Forward | 5′-gagtctggggtccacagttac-3′ | (SEQ ID NO:314) | 21 | 1588 |
| Probe | TET-5′-acctggtacccatggaccgttctgt-3′-TAMRA | (SEQ ID NO:315) | 25 | 1623 |
| Reverse | 5′-ccatcacagagaaggtggtatt-3′ | (SEQ ID NO:316) | 22 | 1654 |

TABLE YC
CNS_neurodegeneration_v1.0

| | Rel. Exp.(%) Ag3530, | | Rel. Exp.(%) Ag3530, |
|---|---|---|---|
| Tissue Name | Run 210631247 | Tissue Name | Run 210631247 |
| AD 1 Hippo | 23.7 | Control (Path) 3 | 6.3 |
| | | Temporal Ctx | |
| AD 2 Hippo | 52.1 | Control (Path) 4 | 53.2 |
| | | Temporal Ctx | |
| AD 3 Hippo | 10.2 | AD 1 Occipital Ctx | 25.3 |
| AD 4 Hippo | 24.7 | AD 2 Occipital Ctx | 0.0 |
| | | (Missing) | |
| AD 5 hippo | 46.0 | AD 3 Occipital Ctx | 10.2 |
| AD 6 Hippo | 51.8 | AD 4 Occipital Ctx | 12.4 |
| Control 2 Hippo | 23.2 | AD 5 Occipital Ctx | 2.4 |
| Control 4 Hippo | 25.9 | AD 6 Occipital Ctx | 18.3 |
| Control (Path) 3 | 8.5 | Control 1 Occipital | 8.7 |
| Hippo | | Ctx | |
| AD 1 Temporal Ctx | 13.8 | Control 2 Occipital | 31.2 |
| | | Ctx | |
| AD 2 Temporal Ctx | 20.7 | Control 3 Occipital | 14.5 |
| | | Ctx | |
| AD 3 Temporal Ctx | 14.4 | Control 4 Occipital | 18.9 |
| | | Ctx | |
| AD 4 Temporal Ctx | 37.6 | Control (Path) 1 | 100.0 |
| | | Occipital Ctx | |
| AD 5 Inf Temporal | 63.3 | Control (Path) 2 | 24.7 |
| Ctx | | Occipital Ctx | |
| AD 5 Sup Temporal | 72.2 | Control (Path) 3 | 7.2 |
| Ctx | | Occipital Ctx | |
| AD 6 Inf Temporal | 48.3 | Control (Path) 4 | 20.3 |
| Ctx | | Occipital Ctx | |
| AD 6 Sup Temporal | 61.6 | Control 1 Parietal | 24.5 |
| Ctx | | Ctx | |
| Control 1 Temporal | 7.8 | Control 2 Parietal | 70.2 |
| Ctx | | Ctx | |
| Control 2 Temporal | 19.9 | Control 3 Parietal | 23.0 |
| Ctx | | Ctx | |
| Control 3 Temporal | 9.0 | Control (Path) 1 | 79.6 |
| Ctx | | Parietal Ctx | |
| Control 4 Temporal | 6.4 | Control (Path) 2 | 37.6 |
| Ctx | | Parietal Ctx | |
| Control (Path) 1 | 39.8 | Control (Path) 3 | 0.0 |
| Temporal Ctx | | Parietal Ctx | |
| Control (Path) 2 | 35.1 | Control (Path) 4 | 37.1 |
| Temporal Ctx | | Parietal Ctx | |

TABLE YD
Panel 4D

| | Rel. | Rel. | | Rel. | Rel. |
|---|---|---|---|---|---|
| | Exp.(%) | Exp.(%) | | Exp.(%) | Exp.(%) |
| | Ag1932, | Ag3530, | | Ag1932, | Ag3530, |
| | Run | Run | | Run | Run |
| Tissue Name | 161561619 | 166446356 | Tissue Name | 161561619 | 166446356 |
| Secondary Th1 act | 0.0 | 0.0 | HUVEC IL-1 beta | 0.0 | 0.0 |
| Secondary Th2 act | 0.0 | 0.0 | HUVEC IFN | 0.0 | 0.0 |
| | | | gamma | | |
| Secondary Tr1 act | 0.0 | 0.0 | HUVEC TNF | 0.0 | 0.0 |
| | | | alpha + IFN | | |
| | | | gamma | | |
| Secondary Th1 rest | 0.0 | 0.0 | HUVEC TNF | 0.0 | 0.0 |
| | | | alpha + IL4 | | |
| Secondary Th2 rest | 0.0 | 0.0 | HUVEC IL-11 | 0.0 | 0.0 |
| Secondary Tr1 rest | 0.0 | 0.0 | Lung | 0.0 | 0.0 |
| | | | Microvascular EC | | |
| | | | none | | |
| Primary Th1 act | 1.7 | 0.0 | Lung | 0.0 | 0.0 |
| | | | Microvascular EC | | |
| | | | TNF alpha + IL- | | |
| | | | 1 beta | | |
| Primary Th2 act | 0.0 | 1.3 | Microvascular | 0.0 | 0.0 |
| | | | Dermal EC none | | |
| Primary Tr1 act | 0.0 | 0.0 | Microsvasular | 0.0 | 0.0 |
| | | | Dermal EC | | |
| | | | TNF alpha + IL- | | |
| | | | 1 beta | | |
| Primary Th1 rest | 0.0 | 0.0 | Bronchial | 0.0 | 0.0 |
| | | | epithelium | | |
| | | | TNF alpha + | | |
| | | | IL1 beta | | |
| Primary Th2 rest | 2.9 | 0.0 | Small airway | 2.3 | 0.0 |
| | | | epithelium none | | |
| Primary Tr1 rest | 3.0 | 2.0 | Small airway | 0.0 | 0.0 |
| | | | epithelium | | |
| | | | TNF alpha + IL- | | |
| | | | 1 beta | | |
| CD45RA CD4 | 1.5 | 3.5 | Coronery artery | 62.9 | 0.0 |
| lymphocyte act | | | SMC rest | | |
| CD45RO CD4 | 0.0 | 0.0 | Coronery artery | 0.0 | 0.0 |
| lymphocyte act | | | SMC TNF alpha + | | |
| | | | IL-1 beta | | |
| CD8 lymphocyte | 0.0 | 0.0 | Astrocytes rest | 12.9 | 1.2 |
| act | | | | | |
| Secondary CD8 | 0.0 | 0.0 | Astrocytes | 4.3 | 6.1 |
| lymphocyte rest | | | TNF alpha + IL- | 4.3 | 6.1 |
| | | | 1 beta | | |
| Secondary CD8 | 1.7 | 0.0 | KU-812 (Basophil) | 36.9 | 7.2 |
| lymphocyte act | | | rest | | |
| CD4 lymphocyte | 8.9 | 2.4 | KU-812 (Basophil) | 23.0 | 14.2 |
| none | | | PMA/ionomycin | | |
| 2ry | 0.0 | 1.0 | CCD1106 | 0.0 | 0.0 |
| Th1/Th2/Tr1_anti- | | | (Keratinocytes) | | |
| CD95 CH11 | | | none | | |
| LAK cells rest | 1.7 | 2.6 | CCD1106 | 0.0 | 0.0 |
| | | | (Keratinocytes) | | |
| | | | TNF alpha + IL- | | |
| | | | 1 beta | | |
| LAK cells IL-2 | 0.0 | 0.0 | Liver cirrhosis | 8.5 | 35.6 |
| LAK cells IL-2 + | 2.1 | 0.8 | Lupus kidney | 4.3 | 4.0 |
| IL-12 | | | | | |
| LAK cells IL- | 0.0 | 1.5 | NCI-H292 none | 0.0 | 0.0 |
| 2 + IFN gamma | | | | | |
| LAK cells IL-2 + | 0.7 | 5.7 | NCI-H292 IL-4 | 6.4 | 0.0 |
| IL-18 | | | | | |
| LAK cells | 0.0 | 0.0 | NCI-H292 IL-9 | 0.0 | 0.0 |
| PMA/ionomycin | | | | | |
| NK Cells IL-2 rest | 0.0 | 0.0 | NCI-H292 IL-13 | 0.0 | 0.0 |
| Two Way MLR 3 | 5.8 | 8.0 | NCI-H292 IFN | 11.7 | 0.0 |
| day | | | gamma | | |
| Two Way MLR 5 | 1.7 | 0.0 | HPAEC none | 0.0 | 0.0 |
| day | | | | | |
| Two Way MLR 7 | 0.0 | 0.0 | HPAEC TNF | 0.0 | 0.0 |
| day | | | alpha + IL-1 beta | | |
| PBMC rest | 19.5 | 4.5 | Lung fibroblast | 0.0 | 0.0 |
| | | | none | | |
| PBMC PWM | 0.5 | 0.0 | Lung fibroblast | 0.0 | 0.0 |
| | | | TNF alpha + IL-1 | | |
| | | | beta | | |
| PBMC PHA-L | 0.0 | 2.8 | Lung fibroblast IL-4 | 0.0 | 0.0 |
| Ramos (B cell) | 4.0 | 50.3 | Lung fibroblast IL-9 | 0.0 | 0.0 |
| none | | | | | |
| Ramos (B cell) | 16.2 | 9.6 | Lung fibroblast IL- | 0.0 | 0.0 |
| ionomycin | | | 13 | | |
| B lymphocytes | 4.7 | 7.0 | Lung fibroblast | 0.0 | 1.2 |
| PWM | | | IFN gamma | | |
| B lymphocytes | 26.6 | 90.1 | Dermal fibroblast | 0.0 | 0.0 |
| CD40L and IL-4 | | | CCD1070 rest | | |
| EOL-1 dbcAMP | 0.0 | 0.6 | Dermal fibroblast | 6.6 | 2.4 |
| | | | CCD1070 TNF | | |
| | | | alpha | | |
| EOL-1 dbcAMP | 3.2 | 0.0 | Dermal fibroblast | 0.0 | 0.0 |
| PMA/ionomycin | | | CCD1070 IL-1 | | |
| | | | beta | | |
| Dendritic cells none | 69.3 | 50.0 | Dermal fibroblast | 0.0 | 0.0 |
| | | | IFN gamma | | |
| Dendritic cells LPS | 4.8 | 10.0 | Dermal fibroblast | 2.4 | 0.0 |
| | | | IL-4 | | |
| Dendritic cells anti- | 95.9 | 99.3 | IBD Colitis 2 | 0.0 | 2.8 |
| CD40 | | | | | |
| Monocytes rest | 20.6 | 7.9 | IBD Crohn's | 0.0 | 0.0 |
| Monocytes LPS | 4.4 | 0.0 | Colon | 39.5 | 100.0 |
| Macrophages rest | 0.0 | 0.0 | Lung | 13.1 | 12.0 |
| Macrophages LPS | 0.0 | 0.0 | Thymus | 100.0 | 8.9 |
| HUVEC none | 0.0 | 0.0 | Kidney | 8.4 | 5.5 |
| HUVEC starved | 0.0 | 0.0 | | | |

CNS_neurodegeneration_v1.0 Summary: Ag3530 This panel confirms the expression of CG59289-01 gene at low levels in the brains of an independent group of individuals. However, no differential expression of this gene was detected between Alzheimer's diseased postmortem brains and those of non-demented controls in this experiment.

The CG59289-01 gene codes for Drosophila CRUMB1 (CRB1) homologue, a protein essential for establishing and maintaining epithelial polarity. In mouse, Crb1 was shown to be expressed exclusively in the eye, and the central nervous system (Ref. 1). Therefore, similar to the mouse orthologue, the CG59289-01 gene may be expressed in eye and central nervous system and may play a role in retinal and central nervous system disorders such as retinal dystrophies, Alzheimer's disease, Parkinson's disease, epilepsy, multiple sclerosis, schizophrenia and depression.

Reference.

1. den Hollander A I, Ghiani M, de Kok Y J, Wijnholds J, Ballabio A, Cremers F P, Broccoli V. (2002) Isolation of Crb1, a mouse homologue of Drosophila crumbs, and analysis of its expression pattern in eye and brain. Mech Dev 110(1-2):203-7

General_screening_panel_v1.4 Summary: Ag3530 Results from one experiment with the CG59289-01 gene are not included. The amp plot indicates that there were experimental difficulties with this run.

Panel 4D Summary: Ag3530 Highest expression of the CG59289-01 gene is seen in normal colon (CT=31). Therefore, expression of this gene may be used to distinguish colon from the other tissues on this panel. Furthermore, expression of this gene is decreased in colon samples from patients with IBD colitis and Crohn's disease relative to normal colon. Therefore, therapeutic modulation of the activity of the protein encoded by this gene may be useful in the treatment of inflammatory bowel disease.

Significant expression is also seen in dendritic cells (DC) and is upregulated in response to CD40. In addition, expression in colon may also result from dendritic cells present in these tissue. Therefore, therapeutic utilization of the protein encoded by this transcript may be important in the treatment of diseases where antigen presentation, a function of mature dendritic cells, plays an important role such as asthma, rheumatoid arthritis, IBD, and psoriasis.

Results from a second experiment with the probe primer set Ag1932 are not included. The amp plot indicates that there were experimental difficulties with this run.

### Z. NOV32a (CG57111-01: PROTOCADHERIN)

Expression of gene CG57111-01 was assessed using the primer-probe sets Ag3242, Ag1012, Ag1096 and Ag704, described in Tables ZA, ZB, ZC and ZD. Results of the RTQ-PCR runs are shown in Tables ZE, ZF, ZG, ZH, ZI, ZJ, and ZK.

TABLE ZA
Probe Name Ag3242

| | | | | Start |
|---|---|---|---|---|
| Primers | Sequences | | Length | Position |
| Forward | 5′-gggaccaatgctcaaattactt-3′ | (SEQ ID NO:317) | 22 | 1038 |
| Probe | TET-5′-tcagaaagttccacaagcatctaagga-3′-TAMRA | (SEQ ID NO:318) | 27 | 1070 |
| Reverse | 5′-atgactccagtgttttcatcca-3′ | (SEQ ID NO:319) | 22 | 1108 |

TABLE ZB
Probe Name Ag1012

| | | | | Start |
|---|---|---|---|---|
| Primers | Sequences | | Length | Position |
| Forward | 5′-catcatcatagctgaggatggt-3′ | (SEQ ID NO:320) | 22 | 0 |
| Probe | TET-5′-acttttgggcagtgcactctcaccat-3′-TAMRA | (SEQ ID NO:321) | 26 | 0 |
| Reverse | 5′-agggcaattgtcattaatgtca-3′ | (SEQ ID NO:322) | 22 | 923 |

TABLE ZC
Probe Name Ag1096

| | | | | Start |
|---|---|---|---|---|
| Primers | Sequences | | Length | Position |
| Forward | 5′-actttggaagaggcattgct-3′ | (SEQ ID NO:323) | 20 | 2403 |
| Probe | TET-5′-cagacagattatgggctccatcgctt3′-TAMRA | (SEQ ID NO:324) | 26 | 2424 |
| Reverse | 5′-ctcgggataaccatgatcact-3′ | (SEQ ID NO:325) | 21 | 2463 |

TABLE ZD
Probe Name Ag704

| | | | |
|---|---|---|---|
| | | | Start |
| Primers | Sequences | Length | Position |

| | | | | | | |
|---|---|---|---|---|---|---|
| Forward | 5′-agacttggggaccaatgct-3′ | (SEQ ID NO:326) | 19 | 1031 | | |
| Probe | TET-5′-tcagaaagttccacaagcatctaagga-3′-TAMRA | (SEQ ID NO:327) | 27 | 1070 | 271 | 1070 |
| Reverse | 5′-tgactccagtgtttcatcca-3′ | (SEQ ID NO:328) | 21 | 1108 | | |

TABLE ZE
AI_comprehensive panel_v1.0

| | Rel. Exp.(%) Ag3242, | | Rel. Exp.(%) Ag3242, |
|---|---|---|---|
| Tissue Name | Run 253058919 | Tissue Name | Run 253058919 |
| 110967 COPD-F | 28.9 | 112427 Match Control | 55.9 |
| | | Psoriasis-F | |
| 110980 COPD-F | 6.7 | 112418 Psoriasis-M | 25.7 |
| 110968 COPD-M | 19.9 | 112723 Match Control | 0.0 |
| | | Psoriasis-M | |
| 110977 COPD-M | 8.4 | 112419 Psoriasis-M | 66.0 |
| 110989 Emphysema-F | 49.3 | 112424 Match Control | 42.0 |
| | | Psoriasis-M | |
| 110992 Emphysema-F | 22.8 | 112420 Psoriasis-M | 44.4 |
| 110993 Emphysema-F | 31.0 | 112425 Match Control | 24.3 |
| | | Psoriasis-M | |
| 110994 Emphysema-F | 12.7 | 104689 (MF) OA Bone- | 81.2 |
| | | Backus | |
| 110995 Emphysema-F | 25.5 | 104690 (MF) Adj | 16.6 |
| | | “Normal” Bone-Backus | |
| 110996 Emphysema-F | 15.8 | 104691 (MF) OA | 1.5 |
| | | Synovium-Backus | |
| 110997 Asthma-M | 7.5 | 104692 (BA) OA | 7.4 |
| | | Cartilage-Backus | |
| 111001 Asthma-F | 30.4 | 104694 (BA) OA Bone- | 56.3 |
| | | Backus | |
| 111002 Asthma-F | 45.4 | 104695 (BA) Adj | 31.6 |
| | | “Normal” Bone-Backus | |
| 111003 Atopic | 45.1 | 104696 (BA) OA | 0.0 |
| Asthma-F | | Synovium-Backus | |
| 111004 Atopic | 22.4 | 104700 (SS) OA Bone- | 63.7 |
| Asthma-F | | Backus | |
| 111005 Atopic | 25.3 | 104701 (SS) Adj | 63.3 |
| Asthma-F | | “Normal” Bone-Backus | |
| 111006 Atopic | 13.9 | 104702 (SS) OA | 6.1 |
| Asthma-F | | Synovium-Backus | |
| 111417 Allergy-M | 15.1 | 117093 OA Cartilage | 16.3 |
| | | Rep7 | |
| 112347 Allergy-M | 5.0 | 112672 OA Bone5 | 13.2 |
| 112349 Normal | 1.9 | 112673 OA Synovium5 | 5.4 |
| Lung-F | | | |
| 112357 Normal | 5.1 | 112674 OA Synovial | 7.7 |
| Lung-F | | Fluid cells5 | |
| 112354 Normal | 11.6 | 117100 OA Cartilage | 4.5 |
| Lung-M | | Rep14 | |
| 112374 Crohns-F | 32.5 | 112756 OA Bone9 | 17.1 |
| 112389 Match Control | 14.4 | 112757 OA Synovium9 | 21.8 |
| Crohns-F | | | |
| 112375 Crohns-F | 36.1 | 112758 OA Synovial | 13.3 |
| | | Fluid Cells9 | |
| 112732 Match Control | 0.0 | 117125 RA Cartilage | 28.1 |
| Crohns-F | | Rep2 | |
| 112725 Crohns-M | 45.4 | 113492 Bone2 RA | 34.2 |
| 112387 Match Control | 18.3 | 113493 Synovium2 RA | 8.9 |
| Crohns-M | | | |
| 112378 Crohns-M | 3.8 | 113494 Syn Fluid Cells | 25.7 |
| | | RA | |
| 112390 Match Control | 22.4 | 113499 Cartilage4 RA | 45.1 |
| Crohns-M | | | |
| 112726 Crohns-M | 45.7 | 113500 Bone4 RA | 47.3 |
| 112731 Match Control | 12.6 | 113501 Synovium4 RA | 42.6 |
| Crohns-M | | | |
| 112380 Ulcer Col-F | 28.3 | 113502 Syn Fluid | 25.5 |
| | | Cells4 RA | |
| 112734 Match Control | 0.8 | 113495 Cartilage3 RA | 33.7 |
| Ulcer Col-F | | | |
| 112384 Ulcer Col-F | 100.0 | 113496 Bone3 RA | 38.7 |
| 112737 Match Control | 28.1 | 113497 Synovium3 RA | 18.2 |
| Ulcer Col-F | | | |
| 112386 Ulcer Col-F | 25.5 | 113498 Syn Fluid | 30.8 |
| | | Cells3 RA | |
| 112738 Match Control | 2.1 | 117106 Normal | 0.0 |
| Ulcer Col-F | | Cartilage Rep20 | |
| 112381 Ulcer Col-M | 2.3 | 113663 Bone3 Normal | 2.8 |
| 112735 Match Control | 53.6 | 113664 Synovium3 | 0.0 |
| Ulcer Col-M | | Normal | |
| 112382 Ulcer Col-M | 15.3 | 113665 Syn Fluid | 2.9 |
| | | Cells3 Normal | |
| 112394 Match Control | 10.2 | 117107 Normal | 10.5 |
| Ulcer Col-M | | Cartilage Rep22 | |
| 112383 Ulcer Col-M | 45.4 | 113667 Bone4 Normal | 17.2 |
| 112736 Match Control | 4.0 | 113668 Synovium4 | 17.0 |
| Ulcer Col-M | | Normal | |
| 112423 Psoriasis-F | 90.1 | 113669 Syn Fluid | 36.3 |
| | | Cells4 Normal | |

TABLE ZF
CNS_neurodegeneration_v1.0

| | Rel. | Rel. | Rel. | | Rel. | Rel. | Rel. |
|---|---|---|---|---|---|---|---|
| | Exp.(%) | Exp.(%) | Exp.(%) | | Exp.(%) | Exp.(%) | Exp.(%) |
| | Ag1012, | Ag1096, | Ag3242, | | Ag1012, | Ag1096, | Ag3242, |
| | Run | Run | Run | Tissue | Run | Run | Run |
| Tissue Name | 206989720 | 206231465 | 206533581 | Name | 206989720 | 206231465 | 206533581 |
| AD 1 Hippo | 34.6 | 27.0 | 16.5 | Control | 1.0 | 0.9 | 1.4 |
| | | | | (Path) 3 | | | |
| | | | | Temporal | | | |
| | | | | Ctx | | | |
| AD 2 Hippo | 18.6 | 11.5 | 16.8 | Control | 11.2 | 11.8 | 4.9 |
| | | | | (Path) 4 | | | |
| | | | | Temporal | | | |
| | | | | Ctx | | | |
| AD 3 Hippo | 6.0 | 8.8 | 5.3 | AD 1 | 5.0 | 6.2 | 2.9 |
| | | | | Occipital | | | |
| | | | | Ctx | | | |
| AD 4 Hippo | 2.6 | 2.0 | 1.9 | AD 2 | 0.0 | 0.0 | 0.0 |
| | | | | Occipital | | | |
| | | | | Ctx | | | |
| | | | | (Missing) | | | |
| AD 5 hippo | 44.8 | 46.7 | 36.6 | AD 3 | 4.1 | 5.6 | 2.8 |
| | | | | Occipital | | | |
| | | | | Ctx | | | |
| AD 6 Hippo | 100.0 | 100.0 | 100.0 | AD 4 | 3.8 | 6.3 | 4.1 |
| | | | | Occipital | | | |
| | | | | Ctx | | | |
| Control 2 | 46.3 | 51.1 | 35.1 | AD 5 | 5.7 | 25.0 | 16.8 |
| Hippo | | | | Occipital | | | |
| | | | | Ctx | | | |
| Control 4 | 5.6 | 5.3 | 4.0 | AD 6 | 24.3 | 5.3 | 1.0 |
| Hippo | | | | Occipital | | | |
| | | | | Ctx | | | |
| Control | 4.0 | 4.6 | 1.0 | Control 1 | 0.2 | 0.8 | 0.3 |
| (Path) 3 | | | | Occipital | | | |
| Hippo | | | | Ctx | | | |
| AD 1 | 4.7 | 3.6 | 2.7 | Control 2 | 22.1 | 17.1 | 19.5 |
| Temporal | | | | Occipital | | | |
| Ctx | | | | Ctx | | | |
| AD 2 | 13.3 | 12.5 | 7.5 | Control 3 | 3.9 | 3.1 | 3.5 |
| Temporal | | | | Occipital | | | |
| Ctx | | | | Ctx | | | |
| AD 3 | 0.9 | 2.5 | 1.0 | Control 4 | 1.0 | 1.4 | 0.8 |
| Temporal | | | | Occipital | | | |
| Ctx | | | | Ctx | | | |
| AD 4 | 5.4 | 6.3 | 3.3 | Control | 36.9 | 34.6 | 30.1 |
| Temporal | | | | (Path) 1 | | | |
| Ctx | | | | Occipital | | | |
| | | | | Ctx | | | |
| AD 5 Inf | 80.7 | 74.7 | 59.5 | Control | 2.5 | 2.2 | 0.6 |
| Temporal | | | | (Path) 2 | | | |
| Ctx | | | | Occipital | | | |
| | | | | Ctx | | | |
| AD 5 | 74.2 | 66.0 | 61.6 | Control | 1.0 | 0.7 | 0.4 |
| SupTemporal | | | | (Path) 3 | | | |
| Ctx | | | | Occipital | | | |
| | | | | Ctx | | | |
| AD 6 Inf | 12.1 | 12.2 | 12.2 | Control | 2.1 | 3.2 | 1.4 |
| Temporal | | | | (Path) 4 | | | |
| Ctx | | | | Occipital | | | |
| | | | | Ctx | | | |
| AD 6 Sup | 13.6 | 14.7 | 12.3 | Control 1 | 1.3 | 1.3 | 1.4 |
| Temporal | | | | Parietal | | | |
| Ctx | | | | Ctx | | | |
| Control 1 | 1.6 | 1.9 | 2.2 | Control 2 | 20.3 | 21.8 | 19.6 |
| Temporal | | | | Parietal | | | |
| Ctx | | | | Ctx | | | |
| Control 2 | 27.4 | 21.8 | 22.2 | Control 3 | 5.9 | 5.9 | 5.7 |
| Temporal | | | | Parietal | | | |
| Ctx | | | | Ctx | | | |
| Control 3 | 4.8 | 5.9 | 4.6 | Control | 40.3 | 38.7 | 38.7 |
| Temporal | | | | (Path) 1 | | | |
| Ctx | | | | Parietal | | | |
| | | | | Ctx | | | |
| Control 4 | 1.3 | 1.7 | 0.9 | Control | 8.4 | 9.5 | 4.3 |
| Temporal | | | | (Path) 2 | | | |
| Ctx | | | | Parietal | | | |
| | | | | Ctx | | | |
| Control | 35.1 | 33.9 | 28.7 | Control | 1.4 | 1.4 | 0.5 |
| (Path) 1 | | | | (Path) 3 | | | |
| Temporal | | | | Parietal | | | |
| Ctx | | | | Ctx | | | |
| Control | 15.1 | 17.3 | 12.3 | Control | 15.0 | 16.8 | 15.5 |
| (Path) 2 | | | | (Path) 4 | | | |
| Temporal | | | | Parietal | | | |
| Ctx | | | | Ctx | | | |

TABLE ZG
Panel 1.2

| | Rel. Exp.(%) | Rel. Exp.(%) | | Rel. Exp.(%) | Rel. Exp.(%) |
|---|---|---|---|---|---|
| | Ag1096, | Ag1096, | | Ag1096, | Ag1096, |
| | Run | Run | | Run | Run |
| Tissue Name | 125094875 | 134204525 | Tissue Name | 125094875 | 134204525 |
| Endothelial cells | 0.0 | 0.0 | Renal ca. 786-0 | 0.0 | 0.0 |
| Heart (Fetal) | 4.5 | 5.6 | Renal ca. A498 | 0.0 | 0.0 |
| Pancreas | 0.2 | 0.3 | Renal ca. RXF | 0.1 | 0.1 |
| | | | 393 | | |
| Pancreatic ca. | 0.0 | 0.0 | Renal ca. | 31.4 | 80.1 |
| CAPAN 2 | | | ACHN | | |
| Adrenal Gland | 1.6 | 2.8 | Renal ca. UO- | 0.0 | 0.0 |
| | | | 31 | | |
| Thyroid | 0.1 | 0.4 | Renal ca. TK- | 1.0 | 1.3 |
| | | | 10 | | |
| Salivary gland | 3.0 | 4.8 | Liver | 1.9 | 5.3 |
| Pituitary gland | 2.7 | 6.7 | Liver (fetal) | 0.8 | 1.7 |
| Brain (fetal) | 2.1 | 7.9 | Liver ca. | 0.0 | 0.0 |
| | | | (hepatoblast) | | |
| | | | HepG2 | | |
| Brain (whole) | 9.9 | 39.8 | Lung | 1.7 | 5.9 |
| (amugdala) | 15.0 | 34.2 | Lung (fetal) | 1.8 | 9.9 |
| Brain | 0.8 | 1.9 | Lung ca. (small | 0.4 | 1.2 |
| (cerebellum) | | | cell) LX-1 | | |
| Brain | 39.0 | 73.2 | Lung ca. (small | 0.2 | 0.5 |
| (hippocampus) | | | cell) NCI-H69 | | |
| Brain (thalamus) | 2.5 | 9.2 | Lung ca. (s.cell | 2.3 | 2.6 |
| | | | var.) SHP-77 | | |
| Cerebral Cortex | 27.0 | 56.3 | Lung ca. (large | 50.7 | 100.0 |
| | | | cell) NCI-H460 | | |
| Spinal cord | 2.2 | 4.8 | Lung ca. (non- | 0.1 | 0.0 |
| | | | sin. cell) A549 | | |
| glio/astro U87- | 0.1 | 0.2 | Lung ca. (non- | 5.4 | 4.0 |
| MG | | | s.cell) NCI- | | |
| | | | H23 | | |
| glio/astro U-118- | 0.1 | 0.2 | Lung ca. (non- | 15.3 | 33.2 |
| MG | | | s.cell) HOP-62 | | |
| astrocytoma | 0.0 | 0.0 | Lung ca. (non- | 0.0 | 0.0 |
| SW1783 | | | s.cl) NCI-H522 | | |
| neuro*; met SK- | 6.9 | 7.3 | Lung ca. | 5.4 | 12.5 |
| N-AS | | | (squam.) SW | | |
| | | | 900 | | |
| astrocytoma SF- | 0.0 | 0.0 | Lung ca. | 0.0 | 0.1 |
| 539 | | | (squam.) NCI- | | |
| | | | H596 | | |
| astrocytoma | 7.0 | 6.8 | Mammary | 0.6 | 2.7 |
| SNB-75 | | | gland | | |
| glioma SNB-19 | 2.4 | 2.2 | Breast ca.* | 0.0 | 0.0 |
| | | | (pl.ef) MCF-7 | | |
| glioma U251 | 5.5 | 11.6 | Breast ca.* | 0.0 | 0.0 |
| | | | (pl.ef) MDA- | | |
| | | | MB-231 | | |
| glioma SF-295 | 0.0 | 0.0 | Breast ca.* (pl. | 0.9 | 1.1 |
| | | | ef) T47D | | |
| Heart | 13.2 | 15.5 | Breast ca. BT- | 0.7 | 0.7 |
| | | | 549 | | |
| Skeletal Muscle | 1.1 | 1.2 | Breast ca. | 0.0 | 0.0 |
| | | | MDA-N | | |
| Bone marrow | 0.0 | 0.1 | Ovary | 0.6 | 0.4 |
| Thymus | 0.5 | 1.7 | Ovarian ca. | 0.5 | 0.3 |
| | | | OVCAR-3 | | |
| Spleen | 0.4 | 0.8 | Ovarian ca. | 0.5 | 0.3 |
| | | | OVCAR-4 | | |
| Lymph node | 0.2 | 0.2 | Ovarian ca. | 15.4 | 15.2 |
| | | | OVCAR-5 | | |
| Colorectal | 4.3 | 8.1 | Ovarian ca. | 0.4 | 1.2 |
| Tissue | | | OVCAR-8 | | |
| Stomach | 3.9 | 8.8 | Ovarian ca. | 0.1 | 0.0 |
| | | | IGROV-1 | | |
| Small intestine | 1.9 | 3.7 | Ovarian ca. | 1.9 | 1.3 |
| | | | (ascites) SK- | | |
| | | | OV-3 | | |
| Colon ca.* | 0.0 | 0.1 | Uterus | 2.2 | 10.6 |
| SW480 | | | | | |
| Colon ca.* | 0.1 | 0.2 | Placenta | 8.2 | 10.1 |
| SW620 (SW480 | | | | | |
| met) | | | | | |
| Colon ca. HT29 | 0.0 | 0.1 | Prostate | 3.4 | 3.5 |
| Colon ca. HCT- | 0.0 | 0.0 | Prostate | 2.6 | 3.5 |
| 116 | | | (bone met) | | |
| | | | PC-3 | | |
| Colon ca. CaCo-2 | 0.0 | 0.0 | Testis | 1.3 | 2.1 |
| Colon ca. Tissue | 0.3 | 1.1 | Melanoma | 0.0 | 0.0 |
| (ODO3866) | | | Hs688(A).T | | |
| Colon ca. HCC- | 2.9 | 4.3 | Melanoma* | 0.0 | 0.1 |
| 2998 | | | (met) | | |
| | | | Hs688(B).T | | |
| Gastric ca.* | 0.0 | 0.0 | Melanoma | 0.0 | 0.0 |
| (liver met) NCI- | | | UACC-62 | | |
| N87 | | | | | |
| Bladder | 0.4 | 0.7 | Melanoma | 0.5 | 0.1 |
| | | | M14 | | |
| Trachea | 3.6 | 16.5 | Melanoma | 0.1 | 0.0 |
| | | | LOX IMVI | | |
| Kidney | 13.2 | 2.7 | Melanoma* | 100.0 | 86.5 |
| | | | (met) SK- | | |
| | | | MEL-5 | | |
| Kidney (fetal) | 7.2 | 10.3 | | | |

TABLE ZH
Panel 1.3D

| | Rel. | Rel. | Rel. | | Rel. | Rel. | Rel. |
|---|---|---|---|---|---|---|---|
| | Exp.(%) | Exp.(%) | Exp.(%) | | Exp.(%) | Exp.(%) | Exp.(%) |
| | Ag1012, | Ag1012, | Ag3242, | | Ag1012, | Ag1012, | Ag3242, |
| | Run | Run | Run | Tissue | Run | Run | Run |
| Tissue Name | 157255773 | 165486783 | 165524415 | Name | 157255773 | 165486783 | 165524415 |
| Liver | 0.0 | 0.0 | 0.0 | Kidney | 3.1 | 3.0 | 2.7 |
| adenocarcinoma | | | | (fetal) | | | |
| Pancreas | 0.1 | 0.7 | 1.0 | Renal ca. | 0.0 | 0.0 | 0.0 |
| | | | | 786-0 | | | |
| Pancreatic ca. | 0.0 | 0.0 | 0.0 | Renal ca. | 0.5 | 1.3 | 0.0 |
| CAPAN 2 | | | | A498 | | | |
| Adrenal gland | 0.8 | 0.9 | 0.3 | Renal ca. | 0.3 | 1.2 | 0.4 |
| | | | | RXF 393 | | | |
| Thyroid | 0.2 | 0.0 | 0.0 | Renal ca. | 44.8 | 95.9 | 100.0 |
| | | | | ACHN | | | |
| Salivary gland | 0.4 | 2.4 | 0.6 | Renal ca. | 0.0 | 2.5 | 0.0 |
| | | | | UO-31 | | | |
| Pituitary gland | 2.6 | 3.9 | 4.8 | Renal ca. | 1.1 | 2.4 | 1.1 |
| | | | | TK-10 | | | |
| Brain (fetal) | 6.3 | 11.7 | 11.8 | Liver | 0.2 | 1.6 | 1.0 |
| Brain (whole) | 11.4 | 37.9 | 40.6 | Liver (fetal) | 0.3 | 1.3 | 0.0 |
| Brain (amygdala) | 19.5 | 38.4 | 31.6 | Liver ca. | 0.0 | 0.0 | 0.0 |
| | | | | (hepatoblast) | | | |
| | | | | HepG2 | | | |
| Brain | 0.3 | 3.1 | 3.0 | Lung | 4.2 | 3.9 | 4.8 |
| (cerebellum) | | | | | | | |
| Brain | 100.0 | 78.5 | 76.3 | Lung (fetal) | 3.8 | 2.9 | 2.2 |
| (hippocampus) | | | | | | | |
| Brain (substantia | 1.9 | 6.3 | 2.1 | Lung ca. | 0.9 | 0.7 | 3.7 |
| nigra) | | | | (small cell) | | | |
| | | | | LX-1 | | | |
| Brain (thalamus) | 4.7 | 17.0 | 6.3 | Lung ca. | 0.4 | 1.3 | 0.0 |
| | | | | (small cell) | | | |
| | | | | NCI-H69 | | | |
| Cerebral Cortex | 34.4 | 40.6 | 27.7 | Lung ca. | 16.7 | 17.3 | 16.0 |
| | | | | (s.cell var.) | | | |
| | | | | SHP-77 | | | |
| Spinal cord | 1.9 | 4.2 | 7.7 | Lung ca. | 8.2 | 51.1 | 46.0 |
| | | | | (large | | | |
| | | | | cell) NCI- | | | |
| | | | | H460 | | | |
| glio/astro U87- | 0.4 | 1.5 | 0.6 | Lung ca. | 0.0 | 0.0 | 0.0 |
| MG | | | | (non-sm. | | | |
| | | | | cell) A549 | | | |
| gilo/astro U-118- | 0.3 | 0.0 | 0.5 | Lung ca. | 3.8 | 6.3 | 8.0 |
| MG | | | | (non-s.cell) | | | |
| | | | | NCI-H23 | | | |
| astrocytoma | 0.0 | 0.0 | 0.0 | Lung ca. | 9.5 | 21.6 | 13.6 |
| SW1783 | | | | (non-s.cell) | | | |
| | | | | HOP-62 | | | |
| neuro*; met SK- | 27.4 | 11.9 | 7.5 | Lung ca. | 0.0 | 0.0 | 0.0 |
| N-AS | | | | (non-s.cl) | | | |
| | | | | NCI-H522 | | | |
| astrocytoma SF- | 0.2 | 0.0 | 0.0 | Lung ca. | 8.0 | 19.8 | 18.8 |
| 539 | | | | (squam.) | | | |
| | | | | SW 900 | | | |
| astrocytoma | 12.6 | 15.0 | 8.4 | Lung ca. | 0.0 | 0.6 | 0.0 |
| SNB-75 | | | | (squam.) | | | |
| | | | | NCI-H596 | | | |
| glioma SNB-19 | 2.8 | 3.8 | 3.0 | Mammary | 0.9 | 0.8 | 1.4 |
| | | | | gland | | | |
| glioma U251 | 14.0 | 100.0 | 66.4 | Breast ca.* | 0.0 | 0.0 | 0.0 |
| | | | | (pl.ef) MCF-7 | | | |
| glioma SF-295 | 0.0 | 0.0 | 0.6 | Breast ca.* | 0.0 | 0.0 | 0.0 |
| | | | | (pl.ef) | | | |
| | | | | MDA-MB- | | | |
| | | | | 231 | | | |
| Heart (fetal) | 8.3 | 4.9 | 3.7 | Breast ca.* | 1.1 | 2.0 | 1.4 |
| | | | | (pl.ef) T47D | | | |
| Heart | 1.3 | 2.8 | 1.7 | Breast ca. | 2.0 | 1.2 | 1.6 |
| | | | | BT-549 | | | |
| Skeletal muscle | 36.1 | 11.9 | 6.9 | Breast ca. | 0.0 | 0.0 | 0.0 |
| (fetal) | | | | MDA-N | | | |
| Skeletal muscle | 0.3 | 2.7 | 1.9 | Ovary | 1.1 | 0.0 | 1.5 |
| Bone marrow | 0.0 | 0.0 | 0.0 | Ovarian ca. | 0.4 | 2.1 | 1.4 |
| | | | | OVCAR-3 | | | |
| Thymus | 1.7 | 0.7 | 0.4 | Ovarian ca. | 0.0 | 0.0 | 0.0 |
| | | | | OVCAR-4 | | | |
| Spleen | 0.6 | 1.2 | 0.0 | Ovarian ca. | 17.7 | 20.9 | 24.8 |
| | | | | OVCAR-5 | | | |
| Lymph node | 0.2 | 0.0 | 0.0 | Ovarian ca. | 1.2 | 0.9 | 0.6 |
| | | | | OVCAR-8 | | | |
| Colorectal | 7.0 | 9.1 | 3.5 | Ovarian ca. | 0.0 | 0.0 | 0.0 |
| | | | | IGROV-1 | | | |
| Stomach | 1.4 | 5.6 | 2.5 | Ovarian ca.* | 2.0 | 2.2 | 5.4 |
| | | | | (ascites) SK- | | | |
| | | | | OV-3 | | | |
| Small intestine | 1.0 | 9.4 | 7.2 | Uterus | 3.8 | 12.4 | 15.5 |
| Colon ca. SW480 | 0.0 | 0.0 | 0.0 | Placenta | 3.5 | 4.6 | 1.1 |
| Colon ca.* | 0.5 | 0.0 | 0.0 | Prostate | 0.7 | 3.1 | 1.4 |
| SW620(SW480 | | | | | | | |
| met) | | | | | | | |
| Colon ca. HT29 | 0.0 | 0.0 | 0.0 | Prostate ca.* | 0.4 | 2.5 | 0.4 |
| | | | | (bone | | | |
| | | | | met)PC-3 | | | |
| Colon ca. HCT- | 0.0 | 0.0 | 0.0 | Testis | 2.1 | 1.5 | 2.3 |
| 116 | | | | | | | |
| Colon ca. CaCo-2 | 0.4 | 0.0 | 0.0 | Melanoma | 0.0 | 0.0 | 0.0 |
| | | | | Hs688(A).T | | | |
| Colon ca. | 0.3 | 0.7 | 0.0 | Melanoma* | 0.0 | 0.0 | 0.0 |
| tissue(ODO3866) | | | | (met) | | | |
| | | | | Hs688(B).T | | | |
| Colon ca. HCC- | 12.4 | 5.1 | 2.1 | Melanoma | 0.2 | 0.0 | 0.0 |
| 2998 | | | | UACC-62 | | | |
| Gastric ca.* | 0.0 | 0.0 | 0.0 | Melanoma | 0.0 | 1.5 | 1.6 |
| (liver met) NCI- | | | | M14 | | | |
| N87 | | | | | | | |
| Bladder | 0.3 | 0.7 | 0.0 | Melanoma | 1.6 | 0.0 | 0.0 |
| | | | | LOX IMVI | | | |
| Trachea | 9.2 | 7.5 | 9.0 | Melanoma* | 68.8 | 50.7 | 58.6 |
| | | | | (met) SK- | | | |
| | | | | MEL-5 | | | |
| Kidney | 1.3 | 5.5 | 1.5 | Adipose | 1.1 | 1.2 | 1.5 |

TABLE ZI
Panel 2.2

| | Rel. Exp.(%) | | Rel. Exp.(%) |
|---|---|---|---|
| | Ag3242, Run | | Ag3242, Run |
| Tissue Name | 174443348 | Tissue Name | 174443348 |
| Normal Colon | 4.4 | Kidney Margin | 1.0 |
| | | (OD04348) | |
| Colon cancer (OD06064) | 1.8 | Kidney malignant | 0.4 |
| | | cancer (OD06204B) | |
| Colon Margin | 8.9 | Kidney normal adjacent | 1.8 |
| (OD06064) | | tissue (OD06204E) | |
| Colon cancer (OD06159) | 2.0 | Kidney Cancer | 2.3 |
| | | (OD04450-01) | |
| Colon Margin | 7.1 | Kidney Margin | 0.9 |
| (OD06159) | | (OD04450-03) | |
| Colon cancer (OD06297- | 2.4 | Kidney Cancer 8120613 | 0.0 |
| 04) | | | |
| Colon Margin | 2.2 | Kidney Margin 8120614 | 0.4 |
| (OD06297-05) | | | |
| CC Gr.2 ascend colon | 0.9 | Kidney Cancer 9010320 | 1.4 |
| (ODO3921) | | | |
| CC Margin (ODO3921) | 3.8 | Kidney Margin 9010321 | 1.3 |
| Colon cancer metastasis | 0.7 | Kidney Cancer 8120607 | 0.8 |
| (OD06104) | | | |
| Lung Margin (OD06104) | 0.0 | Kidney Margin 8120608 | 0.2 |
| Colon mets to lung | 1.7 | Normal Uterus | 5.3 |
| (OD04451-01) | | | |
| Lung Margin (OD04451- | 10.1 | Uterine Cancer 064011 | 0.5 |
| 02) | | | |
| Normal Prostate | 0.4 | Normal Thyroid | 0.0 |
| Prostate Cancer | 0.3 | Thyroid Cancer 064010 | 0.7 |
| (OD04410) | | | |
| Prostate Margin | 0.0 | Thyroid Cancer | 0.9 |
| (OD04410) | | A302152 | |
| Normal Ovary | 0.0 | Thyroid Margin | 0.0 |
| | | A302153 | |
| Ovarian cancer | 0.0 | Normal Breast | 0.6 |
| (OD06283-03) | | | |
| Ovarian Margin | 0.0 | Breast Cancer | 0.0 |
| (OD06283-07) | | (OD04566) | |
| Ovarian Cancer 064008 | 1.9 | Breast Cancer 1024 | 2.1 |
| Ovarian cancer | 0.0 | Breast Cancer | 0.0 |
| (OD06145) | | (OD04590-01) | |
| Ovarian Margin | 0.0 | Breast Cancer Mets | 0.0 |
| (OD06145) | | (OD04590-03) | |
| Ovarian cancer | 0.0 | Breast Cancer | 0.4 |
| (OD06455-03) | | Metastasis (OD04655- | |
| | | 05) | |
| Ovarian Margin | 1.2 | Breast Cancer 064006 | 0.7 |
| (OD06455-07) | | | |
| Normal Lung | 2.3 | Breast Cancer 9100266 | 0.7 |
| Invasive poor diff. lung | 1.1 | Breast Margin 9100265 | 0.2 |
| adeno (ODO4945-01 | | | |
| Lung Margin | 1.6 | Breast Cancer A209073 | 0.0 |
| (ODO4945-03) | | | |
| Lung Malignant Cancer | 0.7 | Breast Margin | 0.0 |
| (OD03126) | | A2090734 | |
| Lung Margin (OD03126) | 1.2 | Breast cancer | 0.4 |
| | | (OD06083) | |
| Lung Cancer | 1.2 | Breast cancer node | 0.9 |
| (OD05014A) | | metastasis (OD06083) | |
| Lung Margin | 4.4 | Normal Liver | 1.3 |
| (OD05014B) | | | |
| Lung cancer (OD06081) | 0.8 | Liver Cancer 1026 | 0.3 |
| Lung Margin (OD06081) | 2.8 | Liver Cancer 1025 | 0.0 |
| Lung Cancer (OD04237- | 0.0 | Liver Cancer 6004-T | 0.0 |
| 01) | | | |
| Lung Margin (OD04237- | 6.0 | Liver Tissue 6004-N | 0.0 |
| 02) | | | |
| Ocular Melanoma | 100.0 | Liver Cancer 6005-T | 2.7 |
| Metastasis | | | |
| Ocular Melanoma | 0.7 | Liver Tissue 6005-N | 3.3 |
| Margin (Liver) | | | |
| Melanoma Metastasis | 1.2 | Liver Cancer 064003 | 7.1 |
| Melanoma Margin | 4.9 | Normal Bladder | 0.0 |
| (Lung) | | | |
| Normal Kidney | 0.0 | Bladder Cancer 1023 | 0.0 |
| Kidney Ca, Nuclear | 1.3 | Bladder Cancer | 0.3 |
| grade 2 (OD04338) | | A302173 | |
| Kidney Margin | 0.4 | Normal Stomach | 5.8 |
| (OD04338) | | | |
| Kidney Ca Nuclear grade | 0.6 | Gastric Cancer 9060397 | 0.0 |
| 1/2 (OD04339) | | | |
| Kidney Margin | 1.6 | Stomach Margin | 2.6 |
| (OD04339 | | 9060396 | |
| Kidney Ca, Clear cell | 0.0 | Gastric Cancer 9060395 | 2.2 |
| type (OD04340) | | | |
| Kidney Margin | 1.1 | Stomach Margin | 3.4 |
| (OD04340) | | 9060394 | |
| Kidney Ca, Nuclear | 0.0 | Gastric Cancer 064005 | 0.3 |
| grade 3 (OD04348) | | | |

TABLE ZJ
Panel 4D

| | Rel. Exp.(%) | Rel. Exp.(%) | Rel. Exp.(%) | Rel. Exp.(%) |
|---|---|---|---|---|
| | Ag1012, Run | Ag1012, Run | Ag1096, Run | Ag3242, Run |
| Tissue Name | 157263859 | 164036950 | 160353275 | 164390548 |
| Secondary Th1 act | 0.0 | 0.0 | 0.0 | 0.0 |
| Secondary Th2 act | 0.0 | 0.0 | 0.0 | 0.0 |
| Secondary Tr1 act | 0.0 | 0.0 | 0.0 | 0.0 |
| Secondary Th1 rest | 0.0 | 0.0 | 0.0 | 0.0 |
| Secondary Th2 rest | 0.0 | 0.0 | 0.0 | 0.0 |
| Secondary Tr1 rest | 0.0 | 0.0 | 0.0 | 0.0 |
| Primary Th1 act | 0.0 | 0.0 | 0.0 | 0.0 |
| Primary Th2 act | 0.0 | 0.7 | 0.0 | 0.0 |
| Primary Tr1 act | 0.0 | 0.0 | 0.0 | 0.0 |
| Primary Th1 rest | 0.0 | 0.0 | 0.0 | 0.0 |
| Primary Th2 rest | 0.0 | 0.0 | 0.0 | 0.0 |
| Primary Tr1 rest | 0.7 | 0.0 | 0.0 | 0.0 |
| CD45RA CD4 | 0.0 | 0.9 | 0.0 | 0.0 |
| lymphocyte act | | | | |
| CD45RO CD4 | 0.0 | 0.0 | 0.0 | 0.0 |
| lymphocyte act | | | | |
| CD8 lymphocyte act | 100.0 | 0.0 | 1.3 | 0.0 |
| Secondary CD8 | 0.0 | 0.0 | 0.7 | 0.0 |
| lymphocyte rest | | | | |
| Secondary CD8 | 0.0 | 0.0 | 0.0 | 0.0 |
| lymphocyte act | | | | |
| CD4 lymphocyte none | 0.0 | 0.0 | 0.0 | 0.0 |
| 2ry Th1/Th2/Tr1_anti- | 0.0 | 0.0 | 0.0 | 0.0 |
| CD95 CH11 | | | | |
| LAK cells rest | 0.0 | 0.0 | 0.0 | 0.0 |
| LAK cells IL-2 | 0.0 | 0.0 | 0.0 | 0.0 |
| LAK cells IL-2 + IL-12 | 0.0 | 2.3 | 0.0 | 0.0 |
| LAK cells IL-2 + IFN | 0.0 | 0.0 | 0.8 | 0.0 |
| gamma | | | | |
| LAK cells IL-2 + IL-18 | 0.3 | 0.0 | 0.0 | 0.0 |
| LAK cells | 0.0 | 0.0 | 0.0 | 0.0 |
| PMA/ionomycin | | | | |
| NK Cells IL-2 rest | 0.0 | 1.9 | 0.8 | 0.0 |
| Two Way MLR 3 day | 0.0 | 0.0 | 0.0 | 0.0 |
| Two Way MLR 5 day | 0.0 | 0.0 | 0.0 | 0.0 |
| Two Way MLR 7 day | 0.0 | 0.0 | 1.5 | 0.0 |
| PBMC rest | 0.0 | 0.0 | 0.0 | 0.0 |
| PBMC PWM | 0.0 | 0.0 | 0.0 | 0.0 |
| PBMC PHA-L | 0.0 | 0.0 | 0.0 | 0.0 |
| Ramos (B cell) none | 0.0 | 0.0 | 0.0 | 0.0 |
| Ramos (B cell) | 0.0 | 0.0 | 0.0 | 0.0 |
| ionomycin | | | | |
| B lymphocytes PWM | 0.0 | 0.0 | 0.0 | 0.0 |
| B lymphocytes CD40L | 0.0 | 0.4 | 0.0 | 0.0 |
| and IL-4 | | | | |
| EOL-1 dbcAMP | 0.0 | 0.0 | 0.0 | 0.0 |
| EOL-1 dbcAMP | 0.0 | 0.0 | 0.0 | 0.0 |
| PMA/ionomycin | | | | |
| Dendritic cells none | 0.0 | 0.0 | 0.0 | 0.0 |
| Dendritic cells LPS | 0.0 | 0.0 | 0.0 | 0.0 |
| Dendritic cells anti- | 0.0 | 0.0 | 0.0 | 0.0 |
| CD40 | | | | |
| Monocytes rest | 0.0 | 0.0 | 0.0 | 0.0 |
| Monocytes LPS | 0.0 | 0.0 | 1.7 | 0.0 |
| Macrophages rest | 0.0 | 0.0 | 0.0 | 0.0 |
| Macrophages LPS | 0.6 | 0.0 | 0.0 | 0.0 |
| HUVEC none | 0.7 | 0.0 | 0.0 | 0.0 |
| HUVEC starved | 0.0 | 0.0 | 0.0 | 0.0 |
| HUVEC IL-1 beta | 0.0 | 0.0 | 0.0 | 0.0 |
| HUVEC IFN gamma | 0.0 | 0.0 | 0.4 | 0.0 |
| HUVEC TNF alpha + | 0.0 | 0.0 | 0.0 | 0.0 |
| IFN gamma | | | | |
| HUVEC TNF alpha + | 0.0 | 0.0 | 2.0 | 0.0 |
| IL4 | | | | |
| HUVEC IL-11 | 0.0 | 0.0 | 0.0 | 0.0 |
| Lung Microvascular EC | 0.0 | 0.0 | 0.0 | 0.0 |
| none | | | | |
| Lung Microvascular EC | 0.0 | 0.0 | 0.0 | 0.0 |
| TNF alpha + IL-1 beta | | | | |
| Microvascular Dermal | 0.0 | 0.0 | 0.0 | 0.0 |
| EC none | | | | |
| Microsvasular Dermal | 0.0 | 0.0 | 0.0 | 0.0 |
| EC TNF alpha + IL- | | | | |
| 1 beta | | | | |
| Bronchial epithelium | 0.0 | 1.1 | 0.0 | 0.0 |
| TNF alpha + IL-1 beta | | | | |
| Small airway epithelium | 0.9 | 1.2 | 0.0 | 0.0 |
| none | | | | |
| Small airway epithelium | 12.6 | 9.4 | 6.5 | 11.7 |
| TNF alpha + IL-1 beta | | | | |
| Coronery artery SMC | 0.0 | 0.0 | 0.7 | 0.0 |
| rest | | | | |
| Coronery artery SMC | 0.0 | 0.0 | 0.0 | 0.0 |
| TNF alpha + IL-1 beta | | | | |
| Astrocytes rest | 10.7 | 15.4 | 13.9 | 16.8 |
| AstrocytesTNF alpha + | 3.8 | 9.1 | 3.6 | 5.3 |
| IL-1 beta | | | | |
| KU-812 (Basophil) rest | 1.5 | 0.0 | 0.9 | 1.0 |
| KU-812 (Basophil) | 90.8 | 100.0 | 100.0 | 100.0 |
| PMA/ionomycin | | | | |
| CCD1106 | 0.0 | 0.0 | 0.0 | 0.0 |
| (Keratinocytes) none | | | | |
| CCD1106 | 0.7 | 0.0 | 0.0 | 0.0 |
| (Keratinocytes) | | | | |
| TNF alpha + IL-1 beta | | | | |
| Liver cirrhosis | 8.1 | 5.4 | 1.9 | 3.8 |
| Lupus kidney | 0.0 | 1.1 | 1.3 | 1.4 |
| NCI-H292 none | 44.1 | 58.2 | 42.9 | 48.0 |
| NCI-H292 IL-4 | 21.8 | 19.3 | 14.7 | 14.8 |
| NCI-H292 IL-9 | 40.9 | 60.3 | 40.6 | 36.6 |
| NCI-H292 IL-13 | 5.9 | 7.4 | 12.2 | 7.4 |
| NCI-H292 IFN gamma | 11.3 | 11.8 | 16.8 | 10.9 |
| HPAEC none | 0.0 | 0.0 | 0.0 | 0.0 |
| HPAEC TNF alpha + | 0.0 | 0.0 | 0.0 | 0.0 |
| IL-1 beta | | | | |
| Lung fibroblast none | 0.0 | 0.0 | 0.0 | 1.7 |
| Lung fibroblast TNF | 0.0 | 0.0 | 0.0 | 0.0 |
| alpha + IL-1 beta | | | | |
| Lung fibroblast IL-4 | 0.0 | 0.0 | 1.7 | 0.0 |
| Lung fibroblast IL-9 | 0.0 | 0.0 | 0.0 | 0.0 |
| Lung fibroblast IL-13 | 0.0 | 0.0 | 0.7 | 0.0 |
| Lung fibroblast IFN | 0.0 | 0.0 | 0.0 | 0.0 |
| gamma | | | | |
| Dermal fibroblast | 0.0 | 0.0 | 0.0 | 0.0 |
| CCD1070 rest | | | | |
| Dermal fibroblast | 0.0 | 0.0 | 0.0 | 0.0 |
| CCD1070 TNF alpha | | | | |
| Dermal fibroblast | 0.0 | 0.0 | 0.9 | 0.0 |
| CCD1070 IL-1 beta | | | | |
| Dermal fibroblast IFN | 0.0 | 2.1 | 0.0 | 0.0 |
| gamma | | | | |
| Dermal fibroblast IL-4 | 0.0 | 0.0 | 0.0 | 0.0 |
| IBD Colitis 2 | 0.0 | 0.5 | 0.8 | 2.6 |
| IBD Crohn's | 0.9 | 2.1 | 0.0 | 2.0 |
| Colon | 22.1 | 14.6 | 28.1 | 23.2 |
| Lung | 74.7 | 38.7 | 34.9 | 51.4 |
| Thymus | 23.2 | 27.4 | 35.1 | 28.3 |
| Kidney | 8.0 | 11.7 | 8.2 | 7.4 |

TABLE ZK
Panel CNS_1

| | Rel. Exp.(%) | | Rel. Exp.(%) |
|---|---|---|---|
| | Ag1012, Run | | Ag1012, Run |
| Tissue Name | 171629312 | Tissue Name | 171629312 |
| BA4 Control | 29.1 | BA17 PSP | 1.9 |
| BA4 Control2 | 52.1 | BA17 PSP2 | 3.1 |
| BA4 | 1.4 | Sub Nigra Control | 21.2 |
| Alzheimer's2 | | | |
| BA4 Parkinson's | 24.0 | Sub Nigra Control2 | 5.3 |
| BA4 | 49.7 | Sub Nigra | 18.9 |
| Parkinson's2 | | Alzheimer's2 | |
| BA4 | 19.1 | Sub Nigra | 65.1 |
| Huntington's | | Parkinson's2 | |
| BA4 | 1.3 | Sub Nigra | 45.4 |
| Huntington's2 | | Huntington's | |
| BA4 PSP | 6.2 | Sub Nigra | 100.0 |
| | | Huntington's2 | |
| BA4 PSP2 | 9.7 | Sub Nigra PSP2 | 4.7 |
| BA4 Depression | 13.0 | Sub Nigra | 2.9 |
| | | Depression | |
| BA4 | 2.4 | Sub Nigra | 25.2 |
| Depression2 | | Depression2 | |
| BA7 Control | 36.1 | Glob Palladus | 7.5 |
| | | Control | |
| BA7 Control2 | 22.8 | Glob Palladus | 14.2 |
| | | Control2 | |
| BA7 | 2.4 | Glob Palladus | 7.1 |
| Alzheimer's2 | | Alzheimer's | |
| BA7 Parkinson's | 6.9 | Glob Palladus | 4.7 |
| | | Alzheimer's2 | |
| BA7 | 34.4 | Glob Palladus | 36.1 |
| Parkinson's2 | | Parkinson's | |
| BA7 | 36.6 | Globa Palladus | 10.5 |
| Huntington's | | Parkinson's2 | |
| BA7 | 32.5 | Glob Palladus PSP | 1.1 |
| Huntington's2 | | | |
| BA7 PSP | 26.4 | Glob Palladus PSP2 | 5.4 |
| BA7 PSP2 | 12.9 | Glob Palladus | 1.0 |
| | | Depression | |
| BA7 Depression | 1.1 | Temp Pole Control | 16.5 |
| BA9 Control | 17.0 | Temp Pole Control2 | 59.0 |
| BA9 Control2 | 90.1 | Temp Pole | 1.2 |
| | | Alzheimer's | |
| BA9 Alzheimer's | 0.0 | Temp Pole | 2.7 |
| | | Alzheimer's2 | |
| BA9 | 2.8 | Temp Pole | 8.1 |
| Alzheimer's2 | | Parkinson's | |
| BA9 Parkinson's | 11.8 | Temp Pole | 20.3 |
| | | Parkinson's2 | |
| BA9 | 43.2 | Temp Pole | 30.6 |
| Parkinson's2 | | Huntington's | |
| BA9 | 25.5 | Temp Pole PSP | 1.1 |
| Huntington's | | | |
| BA9 | 3.7 | Temp Pole PSP2 | 2.1 |
| Huntington's 2 | | | |
| BA9 PSP | 10.7 | Temp Pole | 1.6 |
| | | Depression2 | |
| BA9 PSP2 | 0.0 | Cing Gyr Control | 64.6 |
| BA9 Depression | 3.8 | Cing Gyr Control2 | 42.9 |
| BA9 | 8.0 | Cing Gyr | 16.3 |
| Depression2 | | Alzheimer's | |
| BA17 Control | 15.0 | Cing Gyr | 0.7 |
| | | Alzheimer's2 | |
| BA17 Control2 | 24.5 | Cing Gyr | 17.8 |
| | | Parkinson's | |
| BA17 | 2.8 | Cing Gyr | 28.5 |
| Alzheimer's2 | | Parkinson's2 | |
| BA17 | 13.2 | Cing Gyr | 68.8 |
| Parkinson's | | Huntington's | |
| BA17 | 23.3 | Cing Gyr | 18.4 |
| Parkinson's2 | | Huntington's2 | |
| BA17 | 15.3 | Cing Gyr PSP | 9.9 |
| Huntington's | | | |
| BA17 | 5.0 | Cing Gyr PSP2 | 3.2 |
| Huntington's2 | | | |
| BA17 | 1.7 | Cing Gyr | 3.5 |
| Depression | | Depression | |
| BA17 | 19.9 | Cing Gyr | 10.5 |
| Depression2 | | Depression2 | |

AI_comprehensive panel_v1.0 Summary: Ag3242 Expression of the CG59985-01 gene is ubiquitous in this panel, with high expression in samples derived from patients suffering from ulcerative colitis, Crohn's disease and psoriasis (CTs=33). In addition, significant expression is also seen in samples derived from synovium, cartilage and bone of rheumatoid arthritis. Therefore, antibody or small molecule therapies designed with the protein encoded for by this gene may be useful in the treatment of inflammatory bowel diseases and rheumatoid arthritis.

CNS_neurodegeneration_v1.0 Summary: AG1012/Ag1096/Ag3242. Three experiments with three different probe and primer sets produce results that are in excellent agreement, with highest expression of the CG57448-01 gene in the hippocampus of a patient with Alzheimer's disease. No change is detected in the expression of this gene in the postmortem Alzheimer'diseased brain when compared to controls; however this panel confirms the expression of this gene in the CNS in an independent group of patients. See panel 1.2 for a discussion of utility. A third experiment with the probe and primer set Ag704 shows low/undetectable levels of expression (CTs>35). (Data not shown) The data suggest that there is a probability of a probe failure.

Panel 1.2 Summary: Ag1096/Ag3242 Two experiments with two different probe and primer sets produce results that are in excellent agreement, with highest expression of the CG57448-01 gene in cancer cell lines derived from lung cancer and melanoma (CTs=24-25). Significant levels of expression are also seen in a renal cancer cell line, ovarian cancer cell lines and brain cancer cell lines. Thus, expression of this gene could be used to differentiate between these samples and other samples on this panel and as a diagnostic marker for the presence of these cancers. This gene encodes a protein that is homologous to cadherin, a cell-adhesion molecule. Therefore, therapeutic modulation of the expression or function of this gene may be effective in the treatment of lung, renal and melanoma cancers.

Expression of the this gene is also high in many regions of the brain, including the amygdala, thalamus, cerebellum, and cerebral cortex, with highest expression in the hippocampus. Expression is also detected in the spinal cord. Cadherins can act as axon guidance and cell adhesion proteins, specifically during development and in the response to injury (ref 1). Therefore, manipulation of levels of this protein may be of use in inducing a compensatory synaptogenic response to neuronal death in Alzheimer's disease, Parkinson's disease, Huntingdon's disease, spinocerebellar ataxia, progressive supranuclear palsy, ALS, head trauma, stroke, or any other disease/condition associated with neuronal loss.

Among tissues with metabolic function, this gene is moderately expressed in pituitary gland, adrenal gland, thyroid, pancrease, skeletal muscle, and liver, reflecting the widespread role of cadherins in cell-cell adhesion. This expression suggests that this gene product may play a role in normal metabolic and neuroendocrine function and that dysregulated expression of this gene may contribute to metabolic diseases (such as obesity and diabetes) or neuroendocrine disorders.

References:

Ranscht B. (2000) Cadherins: molecular codes for axon guidance and synapse formation. Int. J. Dev. Neurosci. 18: 643-651.

Panel 1.3D Summary: Ag3242 Highest expression of the CG57448-01 gene is seen in a renal cancer cell line (CT=31.1). Significant expression is also seen in cell lines derived from ovarian cancer, lung cancer, brain cancer and melanoma. This is in concordance with the results in the previous panel. Please see Panel 1.2 for discussion of utility of this gene in the treatment of cancer.

As in the previous panel, this gene is also highly expressed in the central nervous system. Please see Panel 1.2 for a fuller discussion of utility of this gene in the central nervous system.

Results from a second experiment with the probe primer set Ag704 are not included. The amp plot indicates that there is high probability of a probe failure.

Panel 2.2 Summary: Ag3242 Highest expression of the CG57448-01 gene is seen in a sample derived from an ocular melanoma metastasis (CT=29). Thus, expression of this gene could be used to differentiate between this sample and other samples on this pane.

Panel 2D Summary: Ag704 Results from one experiment with the CG57448-01 gene are not included. The amp plot indicates that there were experimental difficulties with this run.

Panel 3D Summary: Ag1012 Results from one experiment with the CG57448-01 gene are not included. The amp plot indicates that there were experimental difficulties with this run.

Panel 4D Summary: Ag1096/Ag3242 Two experiments with two different probe and primer sets produce results that are in excellent agreement, with highest expression of the CG57448-01 gene in the basophil cell line (KU-812) treated with PMA/ionomycin (CTs=30-32). Significant expression is also seen in a cluster of treated and untreated samples derived from the muco-epidermoid cell line NCI-H292. Thus, this gene, which encodes a cadherin homolog, is expressed in both a cell line that is often used as a model of airway epithelium (NCI-H292) and a cell line that is a reasonable model for the inflammatory cells that contribute to various inflammatory lung diseases. This suggests that therapeutic modulation of this gene product may reduce or eliminate the symptoms of patients suffering from pathological and inflammatory lung disorders, including chronic obstructive pulmonary disease, asthma, allergy and emphysema.

Low but significant levels of expression are also seen in the samples derived from normal colon, kidney, lung and thymus. This suggests that this gene product may play a role in the homeostasis of these tissues. Therefore, therapeutic modulation of the expression or function of the gene product may be important for maintaining or restoring normal function to this organs during inflammation.

Results from a third experiment with the probe primer set Ag704 are not included. The amp plot indicates that there is a high probability of a probe failure.

Panel CNS_{—}1 Summary: Ag1012 This panel confirms the expression of the CG57448-01 gene at low levels in the brains of an independent group of individuals. Please see Panel 1.2 for a discussion of the potential utility of this gene in treatment of central nervous system disorders.

### AA. NOV33c (CG59363-03: BAB26184 LIKE)

Expression of gene CG59363-03 was assessed using the primer-probe set Ag5254, described in Table AAA.

TABLE AAA
Probe Name Ag5254

| | | | | Start |
|---|---|---|---|---|
| Primers | Sequences | | Length | Position |
| Forward | 5′-gtcggtgctgcgctt-3′ | (SEQ ID NO:329) | 15 | 881 |
| Probe | TET-5′-cctagcctgtcagactgccttggtc-3′-TAMRA | (SEQ ID NO:330) | 25 | 908 |
| Reverse | 5′-agggccctggggaa-3′ | (SEQ ID NO:331) | 14 | 933 |

CNS_neurodegeneration_v.1.0 Summary: Ag5254 Expression of the CG55966-03 gene is low/undetectable in all samples on this panel (CTs>35).

General_screening_panel_v1.5 Summary: Ag5254 Expression of the CG55966-03 gene is low/undetectable in all samples on this panel (CTs>35).

Panel 4.1D Summary: Ag5254 Expression of the CG55966-03 gene is low/undetectable in all samples on this panel (CTs>35).

### AB. NOV34a 9CG59301-01: ANDROGEN RECEPTOR-LIKE)

Expression of gene CG59301-01 was assessed using the primer-probe set Ag3536, described in Table ABA. Results of the RTQ-PCR runs are shown in Tables ABB, ABC, ABD, and ABE.

TABLE ABA
Probe Name Ag3536

| | | | |
|---|---|---|---|
| | | | Start |
| Primers | Sequences | Length | Position |

| | | | | | | | | |
|---|---|---|---|---|---|---|---|---|
| Forward | 5′-cagctctgcccatcttcat-3′ | (SEQ ID NO:332) | 19 | 919 | | | | |
| Probe | TET-5′-ccagctctctggccagtgcctct-3′-TAMRA | (SEQ ID NO:333) | 23 | 957 | (SEQ ID | 23 | 1 | ~" |
| Reverse | 5′-gcaggtggtgacaccatcta-3′ | (SEQ ID NO:334) | 20 | 980 | | | | |

TABLE ABB
CNS_neurodegeneration_v1.0

| | Rel. Exp.(%) Ag3536, | | Rel. Exp.(%) Ag3536, |
|---|---|---|---|
| Tissue Name | Run 210631621 | Tissue Name | Run 210631621 |
| AD 1 Hippo | 51.1 | Control (Path) 3 | 7.5 |
| | | Temporal Ctx | |
| AD 2 Hippo | 48.3 | Control (Path) 4 | 13.4 |
| | | Temporal Ctx | |
| AD 3 Hippo | 37.4 | AD 1 Occipital Ctx | 4.0 |
| AD 4 Hippo | 11.5 | AD 2 Occipital Ctx | 0.0 |
| | | (Missing) | |
| AD 5 hippo | 44.1 | AD 3 Occipital Ctx | 44.4 |
| AD 6 Hippo | 15.3 | AD 4 Occipital Ctx | 26.1 |
| Control 2 Hippo | 54.7 | AD 5 Occipital Ctx | 15.2 |
| Control 4 Hippo | 21.0 | AD 6 Occipital Ctx | 19.6 |
| Control (Path) 3 | 0.0 | Control 1 Occipital | 18.9 |
| Hippo | | Ctx | |
| AD 1 Temporal Ctx | 62.9 | Control 2 Occipital | 11.5 |
| | | Ctx | |
| AD 2 Temporal Ctx | 20.4 | Control 3 Occipital | 70.2 |
| | | Ctx | |
| AD 3 Temporal Ctx | 42.0 | Control 4 Occipital | 21.3 |
| | | Ctx | |
| AD 4 Temporal Ctx | 38.4 | Control (Path) 1 | 37.4 |
| | | Occipital Ctx | |
| AD 5 Inf Temporal | 41.2 | Control (Path) 2 | 25.9 |
| Ctx | | Occipital Ctx | |
| AD 5 Sup Temporal | 67.8 | Control (Path) 3 | 10.1 |
| Ctx | | Occipital Ctx | |
| AD 6 Inf Temporal | 88.9 | Control (Path) 4 | 0.0 |
| Ctx | | Occipital Ctx | |
| AD 6 Sup Temporal | 88.9 | Control 1 Parietal | 9.0 |
| Ctx | | Ctx | |
| Control 1 Temporal | 19.8 | Control 2 Parietal | 100.0 |
| Ctx | | Ctx | |
| Control 2 Temporal | 11.0 | Control 3 Parietal | 13.4 |
| Ctx | | Ctx | |
| Control 3 Temporal | 46.0 | Control (Path) 1 | 46.0 |
| Ctx | | Parietal Ctx | |
| Control 4 Temporal | 23.7 | Control (Path) 2 | 43.5 |
| Ctx | | Parietal Ctx | |
| Control (Path) 1 | 73.7 | Control (Path) 3 | 0.0 |
| Temporal Ctx | | Parietal Ctx | |
| Control (Path) 2 | 27.2 | Control (Path) 4 | 86.5 |
| Temporal Ctx | | Parietal Ctx | |

TABLE ABC
General_screening_panel_v1.4

| | Rel. Exp.(%) Ag3536, | | Rel. Exp.(%) Ag3536, |
|---|---|---|---|
| Tissue Name | Run 213391032 | Tissue Name | Run 213391032 |
| Adipose | 0.0 | Renal ca. TK-10 | 13.9 |
| Melanoma* | 1.6 | Bladder | 9.5 |
| Hs688(A).T | | | |
| Melanoma* | 1.5 | Gastric ca. (liver met.) | 20.3 |
| Hs688(B).T | | NCI-N87 | |
| Melanoma* M14 | 0.0 | Gastric ca. KATO III | 3.6 |
| Melanoma* | 1.2 | Colon ca. SW-948 | 0.9 |
| LOXIMVI | | | |
| Melanoma* SK- | 5.5 | Colon ca. SW480 | 33.9 |
| MEL-5 | | | |
| Squamous cell | 2.0 | Colon ca.* (SW480 | 12.9 |
| carcinoma SCC-4 | | met) SW620 | |
| Testis Pool | 4.8 | Colon ca. HT29 | 6.9 |
| Prostate ca.* (bone | 12.6 | Colon ca. HCT-116 | 6.0 |
| met) PC-3 | | | |
| Prostate Pool | 15.9 | Colon ca. CaCo-2 | 32.3 |
| Placenta | 0.0 | Colon cancer tissue | 1.7 |
| Uterus Pool | 1.1 | Colon ca. SW1116 | 1.5 |
| Ovarian ca. OVCAR-3 | 4.7 | Colon ca. Colo-205 | 0.0 |
| Ovarian ca. SK-OV-3 | 82.4 | Colon ca. SW-48 | 1.3 |
| Ovarian ca. OVCAR-4 | 2.5 | Colon Pool | 1.5 |
| Ovarian ca. OVCAR-5 | 29.5 | Small Intestine Pool | 17.3 |
| Ovarian ca. IGROV-1 | 15.5 | Stomach Pool | 2.9 |
| Ovarian ca. OVCAR-8 | 4.9 | Bone Marrow Pool | 4.1 |
| Ovary | 0.0 | Fetal Heart | 4.1 |
| Breast ca. MCF-7 | 35.8 | Heart Pool | 4.1 |
| Breast ca. MDA- | 4.5 | Lymph Node Pool | 9.8 |
| MB-231 | | | |
| Breast ca. BT 549 | 12.1 | Fetal Skeletal Muscle | 3.0 |
| Breast ca. T47D | 87.1 | Skeletal Muscle Pool | 4.9 |
| Breast ca. MDA-N | 10.2 | Spleen Pool | 4.2 |
| Breast Pool | 2.9 | Thymus Pool | 4.3 |
| Trachea | 2.7 | CNS cancer (glio/astro) | 2.4 |
| | | U87-MG | |
| Lung | 3.2 | CNS cancer (glio/astro) | 14.2 |
| | | U-118-MG | |
| Fetal Lung | 1.4 | CNS cancer (neuro; met) | 0.0 |
| | | SK-N-AS | |
| Lung ca. NCI-N417 | 0.0 | CNS cancer (astro) SF- | 3.7 |
| | | 539 | |
| Lung ca. LX-1 | 28.3 | CNS cancer (astro) | 18.9 |
| | | SNB-75 | |
| Lung ca. NCI-H146 | 3.6 | CNS cancer (glio) SNB- | 11.7 |
| | | 19 | |
| Lung ca. SHP-77 | 4.3 | CNS cancer (glio) SF- | 15.0 |
| | | 295 | |
| Lung ca. A549 | 2.5 | Brain (Amygdala) Pool | 3.5 |
| Lung ca. NCI-H526 | 0.0 | Brain (cerebellum) | 33.7 |
| Lung ca. NCI-H23 | 100.0 | Brain (fetal) | 8.9 |
| Lung ca. NCI-H460 | 8.2 | Brain (Hippocampus) | 5.8 |
| | | Pool | |
| Lung ca. HOP-62 | 3.8 | Cerebral Cortex Pool | 4.1 |
| Lung ca. NCI-H522 | 26.2 | Brain (Substantia nigra) | 4.5 |
| | | Pool | |
| Liver | 0.0 | Brain (Thalamus) Pool | 5.0 |
| Fetal Liver | 3.4 | Brain (whole) | 4.8 |
| Liver ca. HepG2 | 0.9 | Spinal Cord Pool | 0.0 |
| Kidney Pool | 7.3 | Adrenal Gland | 1.2 |
| Fetal Kidney | 6.7 | Pituitary gland Pool | 1.3 |
| Renal ca. 786-0 | 9.6 | Salivary Gland | 0.9 |
| Renal ca. A498 | 4.6 | Thyroid (female) | 1.1 |
| Renal ca. ACHN | 4.3 | Pancreatic ca. CAPAN2 | 2.5 |
| Renal ca. UO-31 | 11.2 | Pancreas Pool | 3.6 |

TABLE ABD
Panel 2.2

| | Rel. Exp.(%) | | Rel. Exp.(%) |
|---|---|---|---|
| | Ag3536, Run | | Ag3536, Run |
| Tissue Name | 173761868 | Tissue Name | 173761868 |
| Normal Colon | 0.0 | Kidney Margin | 11.9 |
| | | (OD04348) | |
| Colon cancer (OD06064) | 0.0 | Kidney malignant | 36.6 |
| | | cancer (OD06204B) | |
| Colon Margin | 0.0 | Kidney normal adjacent | 0.0 |
| (OD06064) | | tissue (OD06204E) | |
| Colon cancer (OD06159) | 0.0 | Kidney Cancer | 10.5 |
| | | (OD04450-01) | |
| Colon Margin | 0.0 | Kidney Margin | 10.7 |
| (OD06159) | | (OD04450-03) | |
| Colon cancer (OD06297- | 0.0 | Kidney Cancer 8120613 | 10.7 |
| 04) | | | |
| Colon Margin | 0.0 | Kidney Margin 8120614 | 0.0 |
| (OD06297-05) | | | |
| CC Gr.2 ascend colon | 0.0 | Kidney Cancer 9010320 | 0.0 |
| (ODO3921) | | | |
| CC Margin (ODO3921) | 0.0 | Kidney Margin 9010321 | 0.0 |
| Colon cancer metastasis | 0.0 | Kidney Cancer 8120607 | 0.0 |
| (OD06104) | | | |
| Lung Margin (OD06104) | 0.0 | Kidney Margin 8120608 | 0.0 |
| Colon mets to lung | 0.0 | Normal Uterus | 0.0 |
| (OD04451-01) | | | |
| Lung Margin (OD04451- | 4.5 | Uterine Cancer 064011 | 9.7 |
| 02) | | | |
| Normal Prostate | 23.3 | Normal Thyroid | 0.0 |
| Prostate Cancer | 0.0 | Thyroid Cancer 064010 | 0.0 |
| (OD04410) | | | |
| Prostate Margin | 32.1 | Thyroid Cancer | 0.0 |
| (OD04410) | | A302152 | |
| Normal Ovary | 11.7 | Thyroid Margin | 0.0 |
| | | A302153 | |
| Ovarian cancer | 9.3 | Normal Breast | 0.0 |
| (OD06283-03) | | | |
| Ovarian Margin | 0.0 | Breast Cancer | 3.3 |
| (OD06283-07) | | (OD04566) | |
| Ovarian Cancer 064008 | 6.4 | Breast Cancer 1024 | 39.0 |
| Ovarian cancer | 0.0 | Breast Cancer | 50.3 |
| (OD06145) | | (OD04590-01) | |
| Ovarian Margin | 0.0 | Breast Cancer Mets | 42.9 |
| (OD06145) | | (OD04590-03) | |
| Ovarian cancer | 9.3 | Breast Cancer | 100.0 |
| (OD06455-03) | | Metastasis (OD04655- | |
| | | 05) | |
| Ovarian Margin | 5.8 | Breast Cancer 064006 | 11.8 |
| (OD06455-07) | | | |
| Normal Lung | 0.0 | Breast Cancer 9100266 | 3.9 |
| Invasive poor diff. lung | 0.0 | Breast Margin 9100265 | 0.0 |
| adeno (ODO4945-01 | | | |
| Lung Margin | 0.0 | Breast Cancer A209073 | 0.0 |
| (ODO4945-03) | | | |
| Lung Malignant Cancer | 0.0 | Breast Margin | 59.5 |
| (OD03126) | | A2090734 | |
| Lung Margin (OD03126) | 11.4 | Breast cancer | 25.3 |
| | | (OD06083) | |
| Lung Cancer | 8.4 | Breast cancer node | 76.8 |
| (OD05014A) | | metastasis (OD06083) | |
| Lung Margin | 0.0 | Normal Liver | 0.0 |
| (OD05014B) | | | |
| Lung cancer (OD06081) | 0.0 | Liver Cancer 1026 | 0.0 |
| Lung Margin (OD06081) | 0.0 | Liver Cancer 1025 | 11.0 |
| Lung Cancer (OD04237- | 0.0 | Liver Cancer 6004-T | 18.8 |
| 01) | | | |
| Lung Margin (OD04237- | 0.0 | Liver Tissue 6004-N | 0.0 |
| 02) | | | |
| Ocular Melanoma | 7.3 | Liver Cancer 6005-T | 12.8 |
| Metastasis | | | |
| Ocular Melanoma | 0.0 | Liver Tissue 6005-N | 0.0 |
| Margin (Liver) | | | |
| Melanoma Metastasis | 0.0 | Liver Cancer 064003 | 0.0 |
| Melanoma Margin | 0.0 | Normal Bladder | 0.0 |
| (Lung) | | | |
| Normal Kidney | 0.0 | Bladder Cancer 1023 | 0.0 |
| Kidney Ca, Nuclear | 0.0 | Bladder Cancer | 0.0 |
| grade 2 (OD04338) | | A302173 | |
| Kidney Margin | 7.9 | Normal Stomach | 0.0 |
| (OD04338) | | | |
| Kidney Ca Nuclear grade | 24.3 | Gastric Cancer 9060397 | 0.0 |
| 1/2 (OD04339) | | | |
| Kidney Margin | 7.1 | Stomach Margin | 0.0 |
| (OD04339) | | 9060396 | |
| Kidney Ca, Clear cell | 0.0 | Gastric Cancer 9060395 | 25.0 |
| type (OD04340) | | | |
| Kidney Margin | 0.0 | Stomach Margin | 0.0 |
| (OD04340) | | 9060394 | |
| Kidney Ca, Nuclear | 0.0 | Gastric Cancer 064005 | 11.5 |
| grade 3 (OD04348) | | | |

TABLE ABE
Panel 4D

| | Rel. Exp.(%) | | Rel. Exp.(%) |
|---|---|---|---|
| | Ag3536, Run | | Ag3536, Run |
| Tissue Name | 166444751 | Tissue Name | 166444751 |
| Secondary Th1 act | 2.2 | HUVEC IL-1 beta | 0.0 |
| Secondary Th2 act | 0.0 | HUVEC IFN gamma | 0.0 |
| Secondary Tr1 act | 0.0 | HUVEC TNF alpha + IFN | 2.3 |
| | | gamma | |
| Secondary Th1 rest | 0.5 | HUVEC TNF alpha + IL4 | 0.9 |
| Secondary Th2 rest | 0.0 | HUVEC IL-11 | 2.8 |
| Secondary Tr1 rest | 3.7 | Lung Microvascular EC | 7.6 |
| | | none | |
| Primary Th1 act | 0.0 | Lung Microvascular EC | 1.9 |
| | | TNF alpha + IL-1 beta | |
| Primary Th2 act | 5.8 | Microvascular Dermal EC | 4.8 |
| | | none | |
| Primary Tr1 act | 0.8 | Microvascular Dermal EC | 1.0 |
| | | TNF alpha + IL-1 beta | |
| Primary Th1 rest | 5.1 | Bronchial epithelium | 9.7 |
| | | TNF alpha + IL1 beta | |
| Primary Th2 rest | 6.3 | Small airway epithelium | 1.2 |
| | | none | |
| Primary Tr1 rest | 4.8 | Small airway epithelium | 6.3 |
| | | TNF alpha + IL-1 beta | |
| CD45RA CD4 | 0.0 | Coronery artery SMC rest | 1.1 |
| lymphocyte act | | | |
| CD45RO CD4 | 2.5 | Coronery artery SMC | 1.0 |
| lymphocyte act | | TNF alpha + IL-1 beta | |
| CD8 lymphocyte act | 1.0 | Astrocytes rest | 5.1 |
| Secondary CD8 | 0.0 | Astrocytes TNF alpha + IL- | 17.3 |
| lymphocyte rest | | 1 beta | |
| Secondary CD8 | 0.0 | KU-812 (Basophil) rest | 0.0 |
| lymphocyte act | | | |
| CD4 lymphocyte none | 0.7 | KU-812 (Basophil) | 0.0 |
| | | PMA/ionomycin | |
| 2ry Th1/Th2/Tr1_anti- | 1.2 | CCD1106 (Keratinocytes) | 4.0 |
| CD95 CH11 | | none | |
| LAK cells rest | 0.0 | CCD1106 (Keratinocytes) | 14.7 |
| | | TNF alpha + IL-1 beta | |
| LAK cells IL-2 | 5.0 | Liver cirrhosis | 33.0 |
| LAK cells IL-2 + IL-12 | 4.1 | Lupus kidney | 10.0 |
| LAK cells IL-2 + IFN | 8.4 | NCI-H292 none | 2.0 |
| gamma | | | |
| LAK cells IL-2 + IL-18 | 4.5 | NCI-H292 IL-4 | 3.8 |
| LAK cells | 1.3 | NCI-H292 IL-9 | 8.1 |
| PMA/ionomycin | | | |
| NK Cells IL-2 rest | 3.8 | NCI-H292 IL-13 | 10.3 |
| Two Way MLR 3 day | 2.0 | NCI-H292 IFN gamma | 5.0 |
| Two Way MLR 5 day | 1.2 | HPAEC none | 5.2 |
| Two Way MLR 7 day | 1.2 | HPAEC TNF alpha + IL-1 | 3.6 |
| | | beta | |
| PBMC rest | 0.0 | Lung fibroblast none | 8.8 |
| PBMC PWM | 2.4 | Lung fibroblast TNF | 2.6 |
| | | alpha + IL-1 beta | |
| PBMC PHA-L | 0.0 | Lung fibroblast IL-4 | 3.3 |
| Ramos (B cell) none | 0.0 | Lung fibroblast IL-9 | 1.1 |
| Ramos (B cell) | 0.0 | Lung fibroblast IL-13 | 0.0 |
| ionomycin | | | |
| B lymphocytes PWM | 1.1 | Lung fibroblast IFN | 0.0 |
| | | gamma | |
| B lymphocytes CD40L | 2.7 | Dermal fibroblast | 3.4 |
| and IL-4 | | CCD1070 rest | |
| EOL-1 dbcAMP | 0.0 | Dermal fibroblast | 9.7 |
| | | CCD1070 TNF alpha | |
| EOL-1 dbcAMP | 1.9 | Dermal fibroblast | 2.6 |
| PMA/ionomycin | | CCD1070 IL-1 beta | |
| Dendritic cells none | 0.0 | Dermal fibroblast IFN | 3.1 |
| | | gamma | |
| Dendritic cells LPS | 0.0 | Dermal fibroblast IL-4 | 0.0 |
| Dendritic cells anti- | 0.8 | IBD Colitis 2 | 1.0 |
| CD40 | | | |
| Monocytes rest | 0.0 | IBD Crohn's | 0.0 |
| Monocytes LPS | 1.8 | Colon | 100.0 |
| Macrophages rest | 1.9 | Lung | 23.8 |
| Macrophages LPS | 0.9 | Thymus | 4.6 |
| HUVEC none | 6.3 | Kidney | 12.7 |
| HUVEC starved | 4.5 | | |

CNS_neurodegeneration_v.1.0 Summary: Ag3536 This panel confirms the expression of the CG59301-01 gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v.1.4 Summary: Ag3536 Highest expression of the CG59301-01 gene is detected in one of the lung cancer cell line NCI-H23 (CT=30.8). In addition, high expression of this gene is also detected in a breast cancer cell line T47D and ovarian cancer cell line SK-OV-3 (CTs=31). Therefore, expression of this gene can be use to distinguish these samples from other samples used in this panel and as marker in detection of these cancer. Also, significant expression of this gene is seen in CNS cancer, colon cancer, gastric cancer, lung cancer, breast cancer, renal cancer and prostate cancer cell lines. Therefore, therapeutic modulation of the activity of this gene or its protein product, through the use of small molecule drugs, protein therapeutics or antibodies, might be beneficial in the treatment of these cancer.

Low expression of this gene is also observed in samples derived from prostate, kidney and bladder. Therefore, therapeutic modulation of the activity of this gene or its protein product, through the use of small molecule drugs, protein therapeutics or antibodies, might be beneficial in the treatment of disease associated with these tissues.

This gene is expressed at low to moderate levels in cerebellum, hippocampus, and fetal brain (CTs=32-35). Therefore, this gene may play a role in central nervous system disorders such as, Parkinson's disease, epilepsy, seizure, ataxia, autism, schizophrenia and depression.

Panel 2.2 Summary: Ag3536 Significant expression of the CG59301-01 gene is seen in two of the breast cancer metastasis samples in this panel (CTs=34-34.5). Therefore, expression of this gene may be used to distinguish breast cancers from the other samples on this panel. Furthermore, therapeutic modulation of the activity of this gene product may be beneficial in the treatment of breast cancer.

Panel 4D Summary: Ag3536 Highest expression of CG59301-01 is detected in colon (CT=30.83). Therefore, expression of this gene may be used to distinguish colon from the other tissues on this panel. Furthermore, expression of this gene is decreased in colon samples from patients with IBD colitis and Crohn's disease (CT>35) relative to normal colon. Therefore, therapeutic modulation of the this gene product may be useful in the treatment of inflammatory bowel disease.

Low to moderate expression of this gene is also seen samples derived from lung, activated and resting primary Th2, IL-2+IFN gamma treated LAK cells, HUVEC, lung microvascular EC, TNFalpha+IL1beta treated bronchial epithelium, TNFalpha+IL1beta treated small airway epithelium, TNFalpha+IL-1beta treated astrocytes, TNFalpha+IL-1beta treated CCF1106 (keratinocytes), lung fibroblast, TNF alpha treated dermal fibroblast CCD1070 cells and in liver cirrhosis samples. Therefore, therapeutic modulation of this gene or its protein product may be beneficial in the treatment of general autoimmunity, allergies, asthma, psoriasis and emphysema.

Panel CNS_{—}1 Summary: Ag3536 Expression of this gene is low/undetectable (CTs>35) across all of the samples on this panel.

### AC. NOV35a (CG59525-01: CARCINOEMBRYONIC ANTIGEN CGM1)

Expression of gene CG59525-01 was assessed using the primer-probe set Ag3457, described in Table ACA. Results of the RTQ-PCR runs are shown in Tables ACB, and ACC.

TABLE ACA
Probe Name Ag3457

| | | | | Start |
|---|---|---|---|---|
| Primers | Sequences | | Length | Position |
| Forward | 5′-tcccatctatgaggaattgcta-3′ | (SEQ ID NO:335) | 22 | 703 |
| Probe | TET-5′-ccatgacacaaacatttactgctggg-3′-TAMRA | (SEQ ID NO:336) | 26 | 727 |
| Reverse | 5′-aagaaaccacatctgctttgtg-3′ | (SEQ ID NO:337) | 22 | 758 |

TABLE ACB
General_screening_panel_v1.4

| | Rel. Exp.(%) Ag3457, | | Rel. Exp.(%) Ag3457, |
|---|---|---|---|
| Tissue Name | Run 217044328 | Tissue Name | Run 217044328 |
| Adipose | 0.0 | Renal ca. TK-10 | 0.0 |
| Melanoma* | 0.0 | Bladder | 0.0 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 4.3 |
| Melanoma* | 0.0 | Colon ca. SW-948 | 0.0 |
| LOXIMVI | | | |
| Melanoma* SK- | 0.0 | Colon ca. SW480 | 100.0 |
| MEL-5 | | | |
| Squamous cell | 0.0 | Colon ca.* (SW480 | 11.7 |
| carcinoma SCC-4 | | met) SW620 | |
| Testis Pool | 5.6 | Colon ca. HT29 | 0.0 |
| Prostate ca.* (bone | 0.0 | Colon ca. HCT-116 | 0.0 |
| met) PC-3 | | | |
| Prostate Pool | 0.0 | Colon ca. CaCo-2 | 0.0 |
| Placenta | 0.0 | Colon cancer tissue | 13.5 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 10.2 |
| Ovarian ca. OVCAR-5 | 0.0 | Small Intestine Pool | 0.0 |
| Ovarian ca. IGROV-1 | 0.0 | Stomach Pool | 0.0 |
| Ovarian ca. OVCAR-8 | 0.0 | Bone Marrow Pool | 4.8 |
| Ovary | 0.0 | Fetal Heart | 5.3 |
| Breast ca. MCF-7 | 0.0 | Heart Pool | 0.0 |
| Breast ca. MDA- | 0.0 | Lymph Node Pool | 8.4 |
| MB-231 | | | |
| Breast ca. BT 549 | 0.0 | Fetal Skeletal Muscle | 0.0 |
| Breast ca. T47D | 16.0 | Skeletal Muscle Pool | 0.0 |
| Breast ca. MDA-N | 5.3 | Spleen Pool | 20.7 |
| Breast Pool | 21.3 | Thymus Pool | 6.5 |
| Trachea | 0.0 | CNS cancer (glio/astro) | 0.0 |
| | | U87-MG | |
| Lung | 6.1 | CNS cancer (glio/astro) | 4.8 |
| | | U-118-MG | |
| Fetal Lung | 15.5 | CNS cancer (neuro;met) | 0.0 |
| | | SK-N-AS | |
| Lung ca. NCI-N417 | 0.0 | CNS cancer (astro) SF- | 0.0 |
| | | 539 | |
| Lung ca. LX-1 | 24.0 | CNS cancer (astro) | 0.0 |
| | | SNB-75 | |
| Lung ca. NCI-H146 | 0.0 | CNS cancer (glio) SNB- | 0.0 |
| | | 19 | |
| Lung ca. SHP-77 | 5.1 | CNS cancer (glio) SF- | 0.0 |
| | | 295 | |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.0 | Brain (fetal) | 3.4 |
| Lung ca. NCI-H460 | 0.0 | Brain (Hippocampus) | 4.0 |
| | | Pool | |
| Lung ca. HOP-62 | 0.0 | Cerebral Cortex Pool | 17.3 |
| Lung ca. NCI-H522 | 0.0 | Brain (Substantia nigra) | 0.0 |
| | | Pool | |
| Liver | 0.0 | Brain (Thalamus) Pool | 0.0 |
| Fetal Liver | 10.7 | Brain (whole) | 0.0 |
| Liver ca. HepG2 | 0.0 | Spinal Cord Pool | 10.6 |
| Kidney Pool | 6.7 | Adrenal Gland | 0.0 |
| Fetal Kidney | 4.6 | Pituitary gland Pool | 0.0 |
| Renal ca.786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 18.3 |

TABLE ACC
Panel 4D

| | Rel. Exp.(%) | | Rel. Exp.(%) |
|---|---|---|---|
| | Ag3457, Run | | Ag3457, Run |
| Tissue Name | 166417095 | Tissue Name | 166417095 |
| Secondary Th1 act | 100.0 | HUVEC IL-1 beta | 0.0 |
| Secondary Th2 act | 24.0 | HUVEC IFN gamma | 0.0 |
| Secondary Tr1 act | 60.3 | HUVEC TNF alpha + IFN | 0.0 |
| | | gamma | |
| Secondary Th1 rest | 76.8 | HUVEC TNF alpha + IL4 | 0.0 |
| Secondary Th2 rest | 55.1 | HUVEC IL-11 | 0.0 |
| Secondary Tr1 rest | 57.4 | Lung Microvascular EC | 8.7 |
| | | none | |
| Primary Th1 act | 11.3 | Lung Microvascular EC | 1.2 |
| | | TNF alpha + IL-1 beta | |
| Primary Th2 act | 28.1 | Microvascular Dermal EC | 6.0 |
| | | none | |
| Primary Tr1 act | 44.4 | Microsvasular Dermal EC | 1.4 |
| | | TNF alpha + IL-1 beta | |
| Primary Th1 rest | 96.6 | Bronchial epithelium | 0.0 |
| | | TNF alpha + IL1 beta | |
| Primary Th2 rest | 52.9 | Small airway epithelium | 0.0 |
| | | none | |
| Primary Tr1 rest | 26.6 | Small airway epithelium | 0.0 |
| | | TNF alpha + IL-1 beta | |
| CD45RA CD4 | 6.6 | Coronery artery SMC rest | 0.0 |
| lymphocyte act | | | |
| CD45RO CD4 | 36.6 | Coronery artery SMC | 0.0 |
| lymphocyte act | | TNF alpha + IL-1 beta | |
| CD8 lymphocyte act | 8.6 | Astrocytes rest | 0.0 |
| Secondary CD8 | 10.7 | Astrocytes TNF alpha + IL- | 0.0 |
| lymphocyte rest | | 1 beta | |
| Secondary CD8 | 27.0 | KU-812 (Basophil) rest | 0.0 |
| lymphocyte act | | | |
| CD4 lymphocyte none | 24.5 | KU-812 (Basophil) | 0.0 |
| | | PMA/ionomycin | |
| 2ry Th1/Th2/Tr1_anti- | 33.2 | CCD1106 (Keratinocytes) | 0.0 |
| CD95 CH11 | | none | |
| LAK cells rest | 7.6 | CCD1106 (Keratinocytes) | 0.0 |
| | | TNF alpha + IL-1 beta | |
| LAK cells IL-2 | 24.0 | Liver cirrhosis | 4.4 |
| LAK cells IL-2 + IL-12 | 18.6 | Lupus kidney | 0.0 |
| LAK cells IL-2 + IFN | 22.2 | NCI-H292 none | 0.0 |
| gamma | | | |
| LAK cells IL-2 + IL-18 | 12.3 | NCI-H292 IL-4 | 0.0 |
| LAK cells | 8.7 | NCI-H292 IL-9 | 0.0 |
| PMA/ionomycin | | | |
| NK Cells IL-2 rest | 12.8 | NCI-H292 IL-13 | 0.0 |
| Two Way MLR 3 day | 11.8 | NCI-H292 IFN gamma | 0.0 |
| Two Way MLR 5 day | 1.0 | HPAEC none | 0.0 |
| Two Way MLR 7 day | 10.7 | HPAEC TNF alpha + IL-1 | 0.0 |
| | | beta | |
| PBMC rest | 14.3 | Lung fibroblast none | 0.0 |
| PBMC PWM | 6.4 | Lung fibroblast TNF | 0.0 |
| | | alpha + IL-1 beta | |
| PBMC PHA-L | 1.0 | Lung fibroblast IL-4 | 0.0 |
| Ramos (B cell) none | 7.3 | Lung fibroblast IL-9 | 0.0 |
| Ramos (B cell) | 6.4 | Lung fibroblast IL-13 | 0.0 |
| ionomycin | | | |
| B lymphocytes PWM | 43.5 | Lung fibroblast IFN | 0.0 |
| | | gamma | |
| B lymphocytes CD40L | 52.9 | Dermal fibroblast | 0.0 |
| and IL-4 | | CCD1070 rest | |
| EOL-1 dbcAMP | 28.9 | Dermal fibroblast | 54.3 |
| | | CCD1070 TNF alpha | |
| EOL-1 dbcAMP | 21.5 | Dermal fibroblast | 0.0 |
| PMA/ionomycin | | CCD1070 IL-1 beta | |
| Dendritic cells none | 15.5 | Dermal fibroblast IFN | 0.0 |
| | | gamma | |
| Dendritic cells LPS | 4.0 | Dermal fibroblast IL-4 | 0.0 |
| Dendritic cells anti- | 31.2 | IBD Colitis 2 | 2.9 |
| CD40 | | | |
| Monocytes rest | 16.0 | IBD Crohn's | 0.0 |
| Monocytes LPS | 0.8 | Colon | 11.3 |
| Macrophages rest | 17.7 | Lung | 11.1 |
| Macrophages LPS | 8.5 | Thymus | 0.8 |
| HUVEC none | 0.0 | Kidney | 15.4 |
| HUVEC starved | 0.0 | | |

CNS_neurodegeneration_v1.0 Summary: Ag3457 Expression of CG59525-01 gene is low/undetectable (CTs>34.5) across all of the samples on this panel.

General_screening_panel_v1.4 Summary: Ag3457 Significant expression of CG59525-01 gene is seen exclusively in a colon cancer cell line (CT=34.02). Therefore, expression of this gene may be used to distinguish colon cancers from the other samples on this panel. Furthermore, therapeutic modulation of the activity of the protein encoded by this gene may be beneficial in the treatment of colon cancer.

Panel 4D Summary: Ag3457 Low to moderate expression of CG59525-01 gene is detected in both resting and activated, primary and secondary Th1, Th2, Tr1 samples, activated secondary CD8 lymphocyte and 2ry Th1/Th2/Tr1_anti-CD95 CH11. In addition, this gene is down-regulated in activated primary Th1 cells (CT=35) as compared to resting primary Th1 cells (CT=32). Therefore, this gene may be important in regulation of T cell activation or participate in the function(s) of these T cells. Therapeutics designed with the protein encoded for by this transcript could be important in regulating T cell function and treating T cell mediated diseases such as asthma, arthritis, psoriasis, IBD, and systemic lupus erythematosus.

CG59525-01 gene codes for a carcinoembryonic antigen-related cell adhesion molecule (CEACAM). CEACAM1, a related protein, has been shown to regulate early maturation and activation of dentritic cells, thereby facilitating priming and polarization of T cell responses (Ref. 1). Low to moderate expression of this gene is also detected in untreated dendritic cells (CT=34) and anti-CD40 treated cells (CT=33). Thus, the protein encoded by this transcript may be important in dendritic cell differentiation and activation and also, in priming and polarization of T cell responses. Therefore, therapeutics designed with the protein encoded by this transcript could be important for the treatment of asthma, emphysema, inflammatory bowel disease, arthritis and psoriasis.

Expression of this gene is stimulated in IL-2, IL-2+IL12, IL-2+IFN gamma treated LAK cells, PWM/CD40L and IL-4 treated B lymphocytes, anti-CD40 treated dendritic cells, and CCD1070 TNF alpha treated dermal fibroblast. Therefore, therapeutic modulation of this gene or its protein product may be beneficial in the treatment of general autoimmunity, psoriasis and emphysema.

References.

1. Kammerer R, Stober D, Singer B B, Obrink B, Reimann J. (2001) Carcinoembryonic antigen-related cell adhesion molecule 1 on murine dendritic cells is a potent regulator of T cell stimulation. J Immunol Jun. 1, 2001; 166(11 ):6537-44

### AD. NOV36a (CG59484-01: TGF-BETA RESISTANCE-ASSOCIATED PROTEIN)

Expression of gene CG59484-01 was assessed using the primer-probe set Ag3448, described in Table ADA. Results of the RTQ-PCR runs are shown in Tables ADB, ADC and ADD.

TABLE ADA
Probe Name Ag3448

| | | | | Start |
|---|---|---|---|---|
| Primers | Sequences | | Length | Position |
| Forward | 5′-cagaacactccgtggtcatc-3′ | (SEQ ID NO:338) | 20 | 1390 |
| Probe | TET-5′-tcacatgtttgctatatcctcatcagg-3′-TAMRA | (SEQ ID NO:339) | 21 | 1419 |
| Reverse | 5′-tttgatxataxxgagxtgaga-3′ | (SEQ ID NO:340) | 21 | 1446 |

TABLE ADB
CNS_neurodegeneration_v1.0

| | Rel. Exp.(%) Ag3448, | | Rel. Exp.(%) Ag3448, |
|---|---|---|---|
| Tissue Name | Run 210375137 | Tissue Name | Run 210375137 |
| AD 1 Hippo | 8.1 | Control (Path) 3 | 3.2 |
| | | Temporal Ctx | |
| AD 2 Hippo | 23.7 | Control (Path) 4 | 23.0 |
| | | Temporal Ctx | |
| AD 3 Hippo | 4.4 | AD 1 Occipital Ctx | 9.2 |
| AD 4 Hippo | 3.8 | AD 2 Occipital Ctx | 0.0 |
| | | (Missing) | |
| AD 5 Hippo | 96.6 | AD 3 Occipital Ctx | 3.0 |
| AD 6 Hippo | 52.1 | AD 4 Occipital Ctx | 13.3 |
| Control 2 Hippo | 19.8 | AD 5 Occipital Ctx | 38.2 |
| Control 4 Hippo | 5.8 | AD 6 Occipital Ctx | 13.1 |
| Control (Path) 3 | 4.3 | Control 1 Occipital | 1.5 |
| Hippo | | Ctx | |
| AD 1 Temporal Ctx | 9.9 | Control 2 Occipital | 66.0 |
| | | Ctx | |
| AD 2 Temporal Ctx | 25.2 | Control 3 Occipital | 9.2 |
| | | Ctx | |
| AD 3 Temporal Ctx | 3.8 | Control 4 Occipital | 4.6 |
| | | Ctx | |
| AD 4 Temporal Ctx | 1.3 | Control (Path) 1 | 79.6 |
| | | Occipital Ctx | |
| AD 5 Inf Temporal | 100.0 | Control (Path) 2 | 8.5 |
| Ctx | | Occipital Ctx | |
| AD 5 Sup Temporal | 41.8 | Control (Path) 3 | 1.9 |
| Ctx | | Occipital Ctx | |
| AD 6 Inf Temporal | 54.3 | Control (Path) 4 | 12.9 |
| Ctx | | Occipital Ctx | |
| AD 6 Sup Temporal | 50.0 | Control 1 Parietal | 3.6 |
| Ctx | | Ctx | |
| Control 1 Temporal | 3.3 | Control 2 Parietal | 36.3 |
| Ctx | | Ctx | |
| Control 2 Temporal | 45.1 | Control 3 Parietal | 14.9 |
| Ctx | | Ctx | |
| Control 3 Temporal | 7.6 | Control (Path) 1 | 78.5 |
| Ctx | | Parietal Ctx | |
| Control 3 Temporal | 6.3 | Control (Path) 2 | 18.3 |
| Ctx | | Parietal Ctx | |
| Control (Path) 1 | 59.5 | Control (Path) 3 | 2.1 |
| Temporal Ctx | | Parietal Ctx | |
| Control (Path) 2 | 32.3 | Control (Path) 4 | 35.1 |
| Temporal Ctx | | Parietal Ctx | |

TABLE ADC
General_screening_panel_v1.4

| | Rel. Exp.(%) Ag3448, | | Rel. Exp.(%) Ag3448, |
|---|---|---|---|
| Tissue Name | Run 217044206 | Tissue Name | Run 217044206 |
| Adipose | 2.2 | Renal ca.TK-10 | 3.1 |
| Melanoma* | 3.6 | Bladder | 5.2 |
| Hs688(A).T | | | |
| Melanoma* | 4.6 | Gastric ca. (liver met.) | 3.5 |
| Hs688(B).T | | NCI-N87 | |
| Melanoma* M14 | 2.8 | Gastric ca. KATO III | 6.7 |
| Melanoma* | 2.7 | Colon ca. SW-948 | 0.7 |
| LOXIMVI | | | |
| Melanoma* SK- | 6.1 | Colon ca. SW480 | 1.8 |
| MEL-5 | | | |
| Squamous cell | 1.3 | Colon ca.* (SW480 | 2.1 |
| carcinoma SCC-4 | | met) SW620 | |
| Testis Pool | 2.6 | Colon ca. HT29 | 1.4 |
| Prostate ca.* (bone | 9.0 | Colon ca. HCT-116 | 5.5 |
| met) PC-3 | | | |
| Prostate Pool | 2.5 | Colon ca. CaCo-2 | 2.0 |
| Placenta | 1.2 | Colon cancer tissue | 2.7 |
| Uterus Pool | 1.8 | Colon ca. SW1116 | 0.7 |
| Ovarian ca. OVCAR-3 | 4.9 | Colon ca. Colo-205 | 0.5 |
| Ovarian ca. SK-OV-3 | 5.8 | Colon ca. SW-48 | 0.6 |
| Ovarian ca. OVCAR-4 | 0.6 | Colon Pool | 100.0 |
| Ovarian ca. OVCAR-5 | 2.6 | Small Intestine Pool | 5.1 |
| Ovarian ca. IGROV-1 | 1.0 | Stomach Pool | 2.9 |
| Ovarian ca. OVCAR-8 | 0.9 | Bone Marrow Pool | 2.7 |
| Ovary | 2.5 | Fetal Heart | 2.8 |
| Breast ca. MCF-7 | 3.2 | Heart Pool | 2.3 |
| Breast ca. MDA- | 3.9 | Lymph Node Pool | 5.5 |
| MB-231 | | | |
| Breast ca. BT 549 | 7.1 | Fetal Skeletal Muscle | 1.3 |
| Breast ca. T47D | 5.6 | Skeletal Muscle Pool | 3.0 |
| Breast ca. MDA-N | 1.4 | Spleen Pool | 5.8 |
| Breast Pool | 5.0 | Thymus Pool | 6.5 |
| Trachea | 3.8 | CNS cancer (glio/astro) | 3.0 |
| | | U87-MG | |
| Lung | 1.0 | CNS cancer (glio/astro) | 17.7 |
| | | U-118-MG | |
| Fetal Lung | 7.0 | CNS cancer (neuro; met) | 4.8 |
| | | SK-N-AS | |
| Lung ca. NCI-N417 | 1.0 | CNS cancer (astro) SF- | 2.3 |
| | | 539 | |
| Lung ca. LX-1 | 2.0 | CNS cancer (astro) | 9.2 |
| | | SNB-75 | |
| Lung ca. NCI-H146 | 4.1 | CNS cancer (glio) SNB- | 1.1 |
| | | 19 | |
| Lung ca. SHP-77 | 7.8 | CNS cancer (glio) SF- | 10.4 |
| | | 295 | |
| Lung ca. A549 | 4.5 | Brain (Amygdala) Pool | 8.0 |
| Lung ca. NCI-H526 | 1.7 | Brain (cerebellum) | 5.4 |
| Lung ca. NCI-H23 | 5.6 | Brain (fetal) | 10.5 |
| Lung ca. NCI-H460 | 2.6 | Brain (Hippocampus) | 9.0 |
| | | Pool | |
| Lung ca. HOP-62 | 1.5 | Cerebral Cortex Pool | 11.4 |
| Lung ca. NCI-H522 | 4.7 | Brain (Substantia nigra) | 9.4 |
| | | Pool | |
| Liver | 0.5 | Brain (Thalamus) Pool | 15.4 |
| Fetal Liver | 5.6 | Brain (whole) | 14.5 |
| Liver ca. HepG2 | 0.9 | Spinal Cord Pool | 4.8 |
| Kidney Pool | 8.6 | Adrenal Gland | 4.7 |
| Fetal Kidney | 3.3 | Pituitary gland Pool | 2.4 |
| Renal ca. 786-0 | 3.2 | Salivary Gland | 1.4 |
| Renal ca. A498 | 0.7 | Thyroid (female) | 1.5 |
| Renal ca. ACHN | 1.8 | Pancreatic ca. CAPAN2 | 2.5 |
| Renal ca. UO-31 | 3.7 | Pancreas Pool | 5.1 |

TABLE ADD
Panel 4D

| | Rel. Exp.(%) | | Rel. Exp.(%) |
|---|---|---|---|
| | Ag3448, Run | | Ag3448, Run |
| Tissue Name | 166397206 | Tissue Name | 166397206 |
| Secondary Th1 act | 29.9 | HUVEC IL-1 beta | 8.7 |
| Secondary Th2 act | 30.4 | HUVEC IFN gamma | 13.2 |
| Secondary Tr1 act | 48.6 | HUVEC TNF alpha + IFN | 7.1 |
| | | gamma | |
| Secondary Th1 rest | 39.8 | HUVEC TNF alpha + IL4 | 8.1 |
| Secondary Th2 rest | 20.7 | HUVEC IL-11 | 5.3 |
| Secondary Tr1 rest | 31.0 | Lung Microvascular EC | 10.8 |
| | | none | |
| Primary Th1 act | 15.0 | Lung Microvascular EC | 8.7 |
| | | TNFalpha + IL-1 beta | |
| Primary Th2 act | 40.9 | Microvascular Dermal EC | 19.8 |
| | | none | |
| Primary Tr1 act | 54.7 | Microsvasular Dermal EC | 7.6 |
| | | TNFalpha + IL-1 beta | |
| Primary Th1 rest | 100.0 | Bronchial epithelium | 5.2 |
| | | TNFalpha + IL1 beta | |
| Primary Th2 rest | 49.0 | Small airway epithelium | 3.6 |
| | | none | |
| Primary Tr1 rest | 37.6 | Small airway epithelium | 20.7 |
| | | TNFalpha + IL-1 beta | |
| CD45RA CD4 | 15.3 | Coronery artery SMC rest | 6.8 |
| lymphocyte act | | | |
| CD45RO CD4 | 36.9 | Coronery artery SMC | 4.9 |
| lymphocyte act | | TNFalpha + IL-1 beta | |
| CD8 lymphocyte act | 21.3 | Astrocytes rest | 13.6 |
| Secondary CD8 | 24.7 | Astrocytes TNFalpha + IL- | 15.2 |
| lymphocyte rest | | 1 beta | |
| Secondary CD8 | 25.3 | KU-812 (Basophil) rest | 6.1 |
| lymphocyte act | | | |
| CD4 lymphocyte none | 18.8 | KU-812 (Basophil) | 23.2 |
| | | PMA/ionomycin | |
| 2ry Th1/Th2/Tr1_anti- | 48.6 | CCD1106 (Keratinocytes) | 4.8 |
| CD95 CH11 | | none | |
| LAK cells rest | 21.3 | CCD1106 (Keratinocytes) | 21.6 |
| | | TNFalpha + IL-1 beta | |
| LAK cells IL-2 | 61.1 | Liver cirrhosis | 8.5 |
| LAK cells IL-2 + IL-12 | 44.4 | Lupus kidney | 11.9 |
| LAK cells IL-2 + IFN | 48.0 | NCI-H292 none | 14.6 |
| gamma | | | |
| LAK cells IL-2 + IL-18 | 40.3 | NCI-H292 IL-4 | 18.2 |
| LAK cells | 11.8 | NCI-H292 IL-9 | 17.9 |
| PMA/ionomycin | | | |
| NK Cells IL-2 rest | 28.3 | NCI-H292 IL-13 | 10.1 |
| Two Way MLR 3 day | 28.1 | NCI-H292 IFN gamma | 13.3 |
| Two Way MLR 5 day | 22.7 | HPAEC none | 6.1 |
| Two Way MLR 7 day | 25.5 | HPAEC TNF alpha + IL-1 | 8.5 |
| | | beta | |
| PBMC rest | 12.5 | Lung fibroblast none | 13.1 |
| PBMC PWM | 28.7 | Lung fibroblast TNF | 14.9 |
| | | alpha + IL-1 beta | |
| PBMC PHA-L | 9.5 | Lung fibroblast IL-4 | 14.7 |
| Ramos (B cell) none | 24.7 | Lung fibroblast IL-9 | 7.6 |
| Ramos (B cell) | 18.0 | Lung fibroblast IL-13 | 9.8 |
| ionomycin | | | |
| B lymphocytes PWM | 18.0 | Lung fibroblast IFN | 15.4 |
| | | gamma | |
| B lymphocytes CD40L | 25.9 | Dermal fibroblast | 24.7 |
| and IL-4 | | CCD1070 rest | |
| EOL-1 dbcAMP | 12.2 | Dermal fibroblast | 54.3 |
| | | CCD1070 TNF alpha | |
| EOL-1 dbcAMP | 10.1 | Dermal fibroblast | 8.8 |
| PMA/ionomycin | | CCD1070 IL-1 beta | |
| Dendritic cells none | 40.1 | Dermal fibroblast IFN | 5.8 |
| | | gamma | |
| Dendritic cells LPS | 24.3 | Dermal fibroblast IL-4 | 13.6 |
| Dendritic cells anti- | 38.7 | IBD Colitis 2 | 3.4 |
| CD40 | | | |
| Monocytes rest | 28.3 | IBD Crohn's | 4.0 |
| Monocytes LPS | 11.8 | Colon | 46.0 |
| Macrophages rest | 40.1 | Lung | 12.2 |
| Macrophages LPS | 14.8 | Thymus | 19.2 |
| HUVEC none | 12.2 | Kidney | 41.5 |
| HUVEC starved | 16.6 | | |

CNS_neurodegeneration_v1.0 Summary: Ag3448 This panel confirms the expression of CG59484-01 gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3448 Highest expression of CG59484-01 gene is detected in colon (CT=24.9). Therefore, expression of this gene can used to distinguish this sample from other samples in the panel. Furthermore, expression of this gene is decreased in colon cancer tissue and the cell lines (Cts=30-32). Therefore, therapeutic modulation of the activity of the protein encoded by this gene may be useful in the treatment of colon related diseases such as colon cancers, Crohn's disease, and ulcerative colitis.

Among tissues with metabolic or endocrine function, this gene is expressed at moderate levels (CTs=29-30) in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

This gene is expressed at significant (CTs=27-29) levels throughout the CNS, including in amygdala, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression. However, moderate levels of expression of this gene are detected in areas outside of the central nervous system, suggesting the possibility of a wider role in intercellular signaling.

Panel 4D Summary: Ag3448 Highest expression of CG59484-01 gene is detected in resting primary Th1 cells. Thus expression of this gene can be used to distinguish this sample from other samples used in this panel. However, moderate levels of expression of this gene are detected in large number of samples used in this panel, suggesting the possibility of a wider role in intercellular signaling.

Interestingly, expression of this gene is decreased in colon samples from patients with IBD colitis (CT=31.22) and Crohn's disease (CT=30.99) relative to normal colon (CT=27.45). Therefore, therapeutic modulation of the activity of the protein encoded by this gene may be useful in the treatment of inflammatory bowel disease.

### AE. NOV38a (CG59454-01: BUTYROPHILIN)

Expression of gene CG59454-01 was assessed using the primer-probe set Ag3444, described in Table AEA. Results of the RTQ-PCR runs are shown in Tables AEB, and AEC.

TABLE ABA
Probe Name Ag3444

| | | | | Start |
|---|---|---|---|---|
| Primers | Sequences | | Length | Position |
| Forward | 5′-gccttccttctgctcaacttt-3′ | (SEQ ID NO:341) | 20 | 73 |
| Probe | TET-5′-cttcagctgctcatgcctcactcag-3′-TAMRA | (SEQ ID NO:342) | 25 | 112 |
| Reverse | 5′-agggtccaagcacagaaaac-3′ | (SEQ ID NO:343) | 20 | 141 |

TABLE AEB
CNS_neurodegeneration_v1.0

| | Rel. Exp.(%) Ag3444, | | Rel. Exp.(%) Ag3444, |
|---|---|---|---|
| Tissue Name | Run 210374886 | Tissue Name | Run 210374886 |
| AD 1 Hippo | 26.8 | Control (Path) 3 | 17.3 |
| | | Temporal Ctx | |
| AD 2 Hippo | 50.7 | Control (Path) 4 | 25.3 |
| | | Temporal Ctx | |
| AD 3 Hippo | 14.6 | AD 1 Occipital Ctx | 27.2 |
| AD 4 Hippo | 12.3 | AD 2 Occipital Ctx | 0.0 |
| | | (Missing) | |
| AD 5 hippo | 75.3 | AD 3 Occipital Ctx | 19.2 |
| AD 6 Hippo | 88.3 | AD 4 Occipital Ctx | 43.5 |
| Control 2 Hippo | 40.3 | AD 5 Occipital Ctx | 15.4 |
| Control 4 Hippo | 56.6 | AD 6 Occipital Ctx | 36.1 |
| Control (Path) 3 | 10.4 | Control 1 Occipital | 14.9 |
| Hippo | | Ctx | |
| AD 1 Temporal Ctx | 39.2 | Control 2 Occipital | 74.7 |
| | | Ctx | |
| AD 2 Temporal Ctx | 37.4 | Control 3 Occipital | 14.6 |
| | | Ctx | |
| AD 3 Temporal Ctx | 11.0 | Control 4 Occipital | 27.7 |
| | | Ctx | |
| AD 4 Temporal Ctx | 34.2 | Control (Path) 1 | 100.0 |
| | | Occipital Ctx | |
| AD 5 Inf Temporal | 72.2 | Control (Path) 2 | 28.3 |
| Ctx | | Occipital Ctx | |
| AD 5 Sup Temporal | 69.3 | Control (Path) 3 | 3.8 |
| Ctx | | Occipital Ctx | |
| AD 6 Inf Temporal | 33.2 | Control (Path) 4 | 22.8 |
| Ctx | | Occipital Ctx | |
| AD 6 Sup Temporal | 50.7 | Control 1 Parietal | 27.7 |
| Ctx | | Ctx | |
| Control 1 Temporal | 24.7 | Control 2 Parietal | 27.5 |
| Ctx | | Ctx | |
| Control 2 Temporal | 45.1 | Control 3 Parietal | 21.6 |
| Ctx | | Ctx | |
| Control 3 temporal | 20.3 | Control (Path) 1 | 66.0 |
| Ctx | | Parietal Ctx | |
| Control 4 Temporal | 12.2 | Control (Path) 2 | 46.0 |
| Ctx | | Parietal Ctx | |
| Control (Path) 1 | 60.3 | Control (Path) 3 | 12.2 |
| Temporal Ctx | | Parietal Ctx | |
| Control (Path) 2 | 58.6 | Control (Path) 4 | 62.9 |
| Temporal Ctx | | Parietal Ctx | |

TABLE AEC
Panel 4D

| | Rel. Exp.(%) | | Rel. Exp.(%) |
|---|---|---|---|
| | Ag3444, Run | | Ag3444, Run |
| Tissue Name | 166397205 | Tissue Name | 166397205 |
| Secondary Th1 act | 11.5 | HUVEC IL-1 beta | 4.1 |
| Secondary Th2 act | 26.4 | HUVEC IFN gamma | 13.0 |
| Secondary Tr1 act | 22.1 | HUVEC TNF alpha + IFN | 20.2 |
| | | gamma | |
| Secondary Th1 rest | 43.8 | HUVEC TNF alpha + IL4 | 9.4 |
| Secondary Th2 rest | 41.2 | HUVEC IL-11 | 2.4 |
| Secondary Tr1 rest | 49.0 | Lung Microvascular EC | 3.8 |
| | | none | |
| Primary Th1 act | 6.7 | Lung Microvascular EC | 20.2 |
| | | TNFalpha + IL-1 beta | |
| Primary Th2 act | 17.6 | Microvascular Dermal EC | 5.8 |
| | | none | |
| Primary Tr1 act | 18.3 | Microsvasular Dermal EC | 18.8 |
| | | TNFalpha + IL-1 beta | |
| Primary Th1 rest | 98.6 | Bronchial epithelium | 4.4 |
| | | TNFalpha + IL1 beta | |
| Primary Th2 rest | 53.2 | Small airway epithelium | 1.5 |
| | | none | |
| Primary Tr1 rest | 29.1 | Small airway epithelium | 5.1 |
| | | TNFalpha + IL-1 beta | |
| CD45RA CD4 | 12.5 | Coronery artery SMC rest | 6.2 |
| lymphocyte act | | | |
| CD45RO CD4 | 22.4 | Coronery artery SMC | 5.5 |
| lymphocyte act | | TNFalpha + IL-1 beta | |
| CD8 lymphocyte act | 18.0 | Astrocytes rest | 3.1 |
| Secondary CD8 | 20.0 | Astrocytes TNFalpha + IL- | 15.3 |
| lymphocyte rest | | 1 beta | |
| Secondary CD8 | 9.7 | KU-812 (Basophil) rest | 3.1 |
| lymphocyte act | | | |
| CD4 lymphocyte none | 36.3 | KU-812 (Basophil) | 8.2 |
| | | PMA/ionomycin | |
| 2ry Th1/Th2/Tr1_anti- | 43.2 | CCD1106 (Keratinocytes) | 1.6 |
| CD95 CH11 | | none | |
| LAK cells rest | 29.3 | CCD1106 (Keratinocytes) | 25.0 |
| | | TNFalpha + IL-1 beta | |
| LAK cells IL-2 | 76.8 | Liver cirrhosis | 10.2 |
| LAK cells IL-2 + IL-12 | 59.5 | Lupus kidney | 6.0 |
| LAK cells IL-2 + IFN | 77.4 | NCI-H292 none | 10.5 |
| gamma | | | |
| LAK cells IL-2 + IL-18 | 61.1 | NCI-H292 IL-4 | 13.3 |
| LAK cells | 8.0 | NCI-H292 IL-9 | 11.0 |
| PMA/ionomycin | | | |
| NK Cells IL-2 rest | 42.0 | NCI-H292 IL-13 | 7.1 |
| Two Way MLR 3 day | 100.0 | NCI-H292 IFN gamma | 18.3 |
| Two Way MLR 5 day | 33.7 | HPAEC none | 3.1 |
| Two Way MLR 7 day | 13.7 | HPAEC TNF alpha + IL-1 | 14.4 |
| | | beta | |
| PBMC rest | 35.8 | Lung fibroblast none | 10.7 |
| PBMC PWM | 20.9 | Lung fibroblast TNF | 60.7 |
| | | alpha + IL-1 beta | |
| PBMC PHA-L | 5.6 | Lung fibroblast IL-1 | 5.7 |
| Ramos (B cell) none | 18.8 | Lung fibroblast IL-9 | 4.3 |
| Ramos (B cell) | 10.4 | Lung fibroblast IL-13 | 4.2 |
| ionomycin | | | |
| B lymphocytes PWM | 16.7 | Lung fibroblast IFN | 18.2 |
| | | gamma | |
| B lymphocytes CD40L | 26.4 | Dermal fibroblast | 7.9 |
| and IL-4 | | CCD1070 rest | |
| EOL-1 dbcAMP | 2.0 | Dermal fibroblast | 36.6 |
| | | CCD1070 TNF alpha | |
| EOL-1 dbcAMP | 0.6 | Dermal fibroblast | 10.2 |
| PMA/ionomycin | | CCD1070 IL-1 beta | |
| Dendritic cells none | 19.2 | Dermal fibroblast IFN | 24.1 |
| | | gamma | |
| Dendritic cells LPS | 10.2 | Dermal fibroblast IL-4 | 7.0 |
| Dendritic cells anti- | 8.1 | IBD Colitis 2 | 3.9 |
| CD40 | | | |
| Monocytes rest | 30.8 | IBD Crohn's | 9.8 |
| Monocytes LPS | 16.7 | Colon | 92.7 |
| Macrophages rest | 22.7 | Lung | 16.5 |
| Macrophages LPS | 14.9 | Thymus | 8.9 |
| HUVEC none | 4.4 | Kidney | 30.1 |
| HUVEC starved | 8.5 | | |

CNS_neurodegeneration_v1.0 Summary: Ag3444 This panel confirms the expression of the CG59454-01 gene at low levels in the brain in an independent group of individuals. However, no differential expression of this gene was detected between Alzheimer's diseased postmortem brains and those of non-demented controls in this experiment.

The CG59454-01 gene codes for butyrophilin protein (BTN), a milk protein. Recently it was demonstrated that active immunization of the dark Agouti rat with native BFN triggers an inflammatory response in the CNS characterized by the formation of scattered meningeal and perivascular infiltrates of T cells and microphages (see Ref. 1 in panel 4D).

General_screening_panel_v1.4 Summary: Ag3444 Results from one experiment with the CG59454-01 gene are not included. The amp plot indicates that there were experimental difficulties with this run.

Panel 4D Summary: Ag3444 Highest expression of CG59454-01 is detected in samples derived from two way MLR 3 day and resting primary Th1 cells (CTs=25). Thus, expression of this gene can be used to distinguish these two samples from other samples in the panel. In addition, expression of this gene is down-regulated in activated primary Th1 cells (CT=29) as compared to resting primary Th1 cells (CT-=25). Thus, therapeutics designed with the protein encoded for by this transcript could be important in treating T-cell mediated diseases such as asthma, emphysema, psoriasis, IBD, systemic lupus erythematosus, autoimmune encephalomyelitis, and parasitic disease.

CG59454-01 gene codes for butyrophilin protein, a milk protein. Recently, butyrophilin has been shown to modulates the encephalitogenic T cell response to myelin oligodendrocytc glycoprotein in experimental autoimmune encephalomyelitis (Ref. 1).

References.

1. Stefferl A, Schubart A, Storch2 M, Amini A, Mather I, Lassmann H, Linington C. (2000) Butyrophilin, a milk protein, modulates the encephalitogenic T cell response to myelin oligodendrocyte glycoprotein in experimental autoimmune encephalomyelitis. J Immunol 165(5):2859-65

### AF. NOV39a (CG59307-01: KIAA1769)

Expression of gene CG59307-01 was assessed using the primer-probc set Ag3539, described in Table AFA. Results of the RTQ-PCR runs are shown in Tables AFB, AFC and AFD.

TABLE AFA
Probe Name Ag3539

| | | | | Start |
|---|---|---|---|---|
| Primers | Sequences | | Length | Position |
| Forward | 5′-ctactcatctatggcccctttg-3′ | (SEQ ID NO:344) | 22 | 2617 |
| Probe | TET-5′-caagacctacacgctggccatgg-3′-TAMRA | (SEQ ID NO:345) | 23 | 2646 |
| Reverse | 5′-agatgagcaccttggtttcag-3′ | (SEQ ID NO:345) | 21 | 2693 |

TABLE AFB
CNS_neurodegeneration_v1.0

| | Rel. Exp.(%) Ag3539, | | Rel. Exp.(%) Ag3539, |
|---|---|---|---|
| Tissue Name | Run 210638376 | Tissue Name | Run 210638376 |
| AD 1 Hippo | 24.7 | Control (Path) 3 | 55.5 |
| | | Temporal Ctx | |
| AD 2 Hippo | 34.9 | Control (Path) 4 | 34.6 |
| | | Temporal Ctx | |
| AD 3 Hippo | 39.5 | AD 1 Occipital Ctx | 27.0 |
| AD 4 Hippo | 48.6 | AD 2 Occipital Ctx | 0.0 |
| | | (Missing) | |
| AD 5 Hippo | 56.3 | AD 3 Occipital Ctx | 39.5 |
| AD 6 Hippo | 85.3 | AD 4 Occipital Ctx | 49.0 |
| Control 2 Hippo | 43.8 | AD 5 Occipital Ctx | 57.4 |
| Control 4 Hippo | 62.9 | AD 6 Occipital Ctx | 70.2 |
| Control (Path) 3 | 24.7 | Control 1 Occipital | 79.6 |
| Hippo | | Ctx | |
| AD 1 Temporal Ctx | 59.9 | Control 2 Occipital | 53.6 |
| | | Ctx | |
| AD 2 Temporal Ctx | 29.1 | Control 3 Occipital | 25.3 |
| | | Ctx | |
| AD 3 Temporal Ctx | 26.8 | Control 4 Occipital | 48.6 |
| | | Ctx | |
| AD 4 Temporal Ctx | 50.3 | Control (Path) 1 | 33.7 |
| | | Occipital Ctx | |
| AD 5 Inf Temporal | 82.9 | Control (Path) 2 | 17.1 |
| Ctx | | Occipital Ctx | |
| AD 5 Sup Temporal | 58.2 | Control (Path) 3 | 26.4 |
| Ctx | | Occipital Ctx | |
| AD 6 Inf Temporal | 98.6 | Control (Path) 4 | 36.9 |
| Ctx | | Occipital Ctx | |
| AD 6 Sup Temporal | 100.0 | Control 1 Parietal | 16.7 |
| Ctx | | Ctx | |
| Control 1 Temporal | 31.2 | Control 2 Parietal | 76.3 |
| Ctx | | Ctx | |
| Control 2 Temporal | 41.2 | Control 3 Parietal | 15.5 |
| Ctx | | Ctx | |
| Control 3 Temporal | 22.2 | Control (Path) 1 | 28.7 |
| Ctx | | Parietal Ctx | |
| Control 3 Temporal | 49.3 | Control (Path) 2 | 23.0 |
| Ctx | | Parietal Ctx | |
| Control (Path) 1 | 53.6 | Control (Path) 3 | 27.0 |
| Temporal Ctx | | Parietal Ctx | |
| Control (Path) 2 | 41.8 | Control (Path) 4 | 52.9 |
| Temporal Ctx | | Parietal Ctx | |

TABLE AFC
General_screening_panel_v1.4

| | Rel. Exp.(%) Ag3539, | | Rel. Exp.(%) Ag3539, |
|---|---|---|---|
| Tissue Name | Run 217049118 | Tissue Name | Run 217049118 |
| Adipose | 2.4 | Renal ca. TK-10 | 3.6 |
| Melanoma* | 1.2 | Bladder | 12.5 |
| Hs688(A).T | | | |
| Melanoma* | 0.9 | Gastric ca. (liver met.) | 100.0 |
| Hs688(B).T | | NCI-N87 | |
| Melanoma* M14 | 2.7 | Gastric ca: KATO III | 5.4 |
| Melanoma* | 2.2 | Colon ca. SW-948 | 1.1 |
| LOXIMVI | | | |
| Melanoma* SK- | 2.9 | Colon ca. SW480 | 8.3 |
| MEL-5 | | | |
| Squamous cell | 1.6 | Colon ca.* (SW480 | 1.8 |
| carcinoma SCC-4 | | met) SW620 | |
| Testis Pool | 0.4 | Colon ca. HT29 | 2.6 |
| Prostate ca.* (bone | 3.3 | Colon ca. HCT-116 | 3.4 |
| met) PC-3 | | | |
| Prostate Pool | 1.2 | Colon ca. CaCo-2 | 4.4 |
| Placenta | 0.7 | Colon cancer tissue | 5.5 |
| Uterus Pool | 0.2 | Colon ca. SW1116 | 0.6 |
| Ovarian ca. OVCAR-3 | 3.3 | Colon ca. Colo-205 | 0.4 |
| Ovarian ca. SK-OV-3 | 1.8 | Colon ca. SW-48 | 1.0 |
| Ovarian ca. OVCAR-4 | 1.5 | Colon Pool | 0.9 |
| Ovarian ca. OVCAR-5 | 9.3 | Small Intestine Pool | 0.8 |
| Ovarian ca. IGROV-1 | 6.3 | Stomach Pool | 0.8 |
| Ovarian ca. OVCAR-8 | 5.8 | Bone Marrow Pool | 0.4 |
| Ovary | 1.7 | Fetal Heart | 0.4 |
| Breast ca. MCF-7 | 7.0 | Heart Pool | 0.6 |
| Breast ca. MDA- | 5.4 | Lymph Node Pool | 0.8 |
| MB-231 | | | |
| Breast ca. BT 549 | 40.9 | Fetal Skeletal Muscle | 0.5 |
| Breast ca. T47D | 21.5 | Skeletal Muscle Pool | 1.7 |
| Breast ca. MDA-N | 2.8 | Spleen Pool | 5.9 |
| Breast Pool | 0.8 | Thymus Pool | 1.7 |
| Trachea | 1.2 | CNS cancer (glio/astro) | 4.1 |
| | | U87-MG | |
| Lung | 0.2 | CNS cancer (glio/astro) | 7.1 |
| | | U-118-MG | |
| Fetal Lung | 3.3 | CNS cancer (neuro;met) | 3.0 |
| | | SK-N-AS | |
| Lung ca. NCI-N417 | 0.0 | CNS cancer (astro) SF- | 1.3 |
| | | 539 | |
| Lung ca. LX-1 | 3.0 | CNS cancer (astro) | 6.8 |
| | | SNB-75 | |
| Lung ca. NCI-H146 | 0.2 | CNS cancer (gilo) SNB- | 5.7 |
| | | 19 | |
| Lung ca. SHP-77 | 1.9 | CNS cancer (glio) SF- | 11.3 |
| | | 295 | |
| Lung ca. A549 | 0.7 | Brain (Amygdala) Pool | 0.2 |
| Lung ca. NCI-H526 | 0.2 | Brain (cerebellum) | 0.3 |
| Lung ca. NCI-H23 | 6.7 | Brain (fetal) | 0.5 |
| Lung ca. NCI-H460 | 0.6 | Brain (Hippocampus) | 0.2 |
| | | Pool | |
| Lung ca. HOP-62 | 2.8 | Cerebral Cortex Pool | 0.3 |
| Lung ca. NCI-H522 | 23 | Brain (Substantia nigra) | 0.5 |
| | | Pool | |
| Liver | 0.4 | Brain (Thalamus) Pool | 0.2 |
| Fetal Liver | 0.6 | Brain (whole) | 0.5 |
| Liver ca. HepG2 | 0.7 | Spinal Cord Pool | 0.4 |
| Kidney Pool | 1.5 | Adrenal Gland | 1.4 |
| Fetal Kidney | 0.5 | Pituitary gland Pool | 0.3 |
| Renal ca. 786-0 | 2.1 | Salivary Gland | 0.5 |
| Renal ca. A498 | 1.4 | Thyroid (female) | 1.2 |
| Renal ca. ACHN | 2.4 | Pancreatic ca. CAPAN2 | 4.0 |
| Renal ca. UO-31 | 1.4 | Pancreas Pool | 1.3 |

TABLE AFD
Panel 4D

| | Rel Exp.(%) | | Rel. Exp.(%) |
|---|---|---|---|
| | Ag3539, Run | | Ag3539, Run |
| Tissue Name | 166447031 | Tissue Name | 166447031 |
| Secondary Th1 act | 6.2 | HUVEC IL-1 beta | 3.4 |
| Secondary Th2 act | 57.0 | HUVEC IFN gamma | 18.6 |
| Secondary Tr1 act | 18.0 | HUVEC TNF alpha + IFN | 44.4 |
| | | gamma | |
| Secondary Th1 rest | 23.0 | HUVEC TNF alpha + IL4 | 14.6 |
| Secondary Th2 rest | 7.3 | HUVEC IL-11 | 3.3 |
| Secondary Tr1 rest | 13.6 | Lung Microvascular EC | 6.8 |
| | | none | |
| Primary Th1 act | 0.5 | Lung Microvascular EC | 14.8 |
| | | TNFalpha + IL-1 beta | |
| Primary Th2 act | 2.4 | Microvascular Dermal EC | 5.9 |
| | | none | |
| Primary Tr1 act | 2.1 | Microsvasular Dermal EC | 17.2 |
| | | TNFalpha + IL-1 beta | |
| Primary Th1 rest | 16.2 | Bronchial epithelium | 7.0 |
| | | TNFalpha + IL1 beta | |
| Primary Th2 rest | 3.6 | Small airway epithelium | 2.4 |
| | | none | |
| Primary Tr1 rest | 2.5 | Small airway epithelium | 16.5 |
| | | TNFalpha + IL-1 beta | |
| CD45RA CD4 | 20.4 | Coronery artery SMC rest | 3.1 |
| lymphocyte act | | | |
| CD45RO CD4 | 12.2 | Coronery artery SMC | 3.3 |
| lymphocyte act | | TNFalpha + IL-1 beta | |
| CD8 lymphocyte act | 2.3 | Astrocytes rest | 3.5 |
| Secondary CD8 | 17.8 | Astrocytes TNFalpha + IL- | 30.6 |
| lymphocyte rest | | 1 beta | |
| Secondary CD8 | 8.1 | KU-812 (Basophil) rest | 0.6 |
| lymphocyte act | | | |
| CD4 lymphocyte none | 7.0 | KU-812 (Basophil) | 6.6 |
| | | PMA/ionomycin | |
| 2ry Th1/Th2/Tr1_anti- | 3.0 | CCD1106 (Keratinocytes) | 9.6 |
| CD95 CH11 | | none | |
| LAK cells rest | 6.8 | CCD1106 (Keratinocytes) | 100.0 |
| | | TNFalpha + IL-1 beta | |
| LAK cells IL-2 | 23.7 | Liver cirrhosis | 5.2 |
| LAK cells IL-2 + IL-12 | 33.4 | Lupus kidney | 4.0 |
| LAK cells IL-2 + IFN | 48.6 | NCI-H292 none | 8.2 |
| gamma | | | |
| LAK cells IL-2 + IL-18 | 35.8 | NCI-H292 IL-4 | 13.0 |
| LAK cells | 19.2 | NCI-H292 IL-9 | 9.4 |
| PMA/ionomycin | | | |
| NK Cells IL-2 rest | 17.0 | NCI-H292 IL-13 | 10.2 |
| Two Way MLR 3 day | 38.4 | NCI-H292 IFN gamma | 37.1 |
| Two Way MLR 5 day | 19.2 | HPAEC none | 2.6 |
| Two Way MLR 7 day | 6.1 | HPAEC TNF alpha + IL-1 | 29.9 |
| | | beta | |
| PBMC rest | 7.7 | Lung fibroblast none | 10.1 |
| PBMC PWM | 8.3 | Lung fibroblast TNF | 61.1 |
| | | alpha + IL-1 beta | |
| PBMC PHA-L | 1.8 | Lung fibroblast IL-4 | 10.3 |
| Ramos (B cell) none | 0.8 | Lung fibroblast IL-9 | 4.8 |
| Ramos (B cell) | 0.3 | Lung fibroblast IL-13 | 5.0 |
| ionomycin | | | |
| B lymphocytes PWM | 4.0 | Lung fibroblast IFN | 41.8 |
| | | gamma | |
| B lymphocytes CD40L | 7.8 | Dermal fibroblast | 4.2 |
| and IL-4 | | CCD1070 rest | |
| EOL-1 dbcAMP | 5.4 | Dermal fibroblast | 10.9 |
| | | CCD1070 TNF alpha | |
| EOL-1 dbcAMP | 10.4 | Dermal fibroblast | 8.4 |
| PMA/ionomycin | | CCD1070 IL-1 beta | |
| Dendritic cells none | 3.3 | Dermal fibroblast IFN | 23.7 |
| | | gamma | |
| Dendritic cells LPS | 41.8 | Dermal fibroblast IL-4 | 10.2 |
| Dendritic cells anti- | 1.9 | IBD Colitis 2 | 2.2 |
| CD40 | | | |
| Monocytes rest | 13.3 | IBD Crohn's | 2.7 |
| Monocytes LPS | 57.0 | Colon | 26.8 |
| Macrophages rest | 3.3 | Lung | 7.3 |
| Macrophages LPS | 60.7 | Thymus | 2.8 |
| HUVEC none | 3.6 | Kidney | 9.2 |
| HUVEC starved | 7.1 | | |

CNS_neurodegeneration_v1.0 Summary: Ag3539 This panel confirms the expression of the CG59307-01 gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3539 Highest expression of the CG59307-0l gene is detected in a sample derived from one of the gastric cancer cell line NCI-N87 (CT=23.9). Thus expression of this gene can be used to distinguish this sample from other samples in the panel. In addition low levels of expression of this gene is also associated with colon cancer, ovarian cancer, breast cancer, and CNS cancer cell lines. Therefore, therapeutic modulation of the activity of this gene or its protein product, through the use of small molecule drugs, protein therapeutics or antibodies, might be beneficial in the treatment of these cancers.

Among tissues with metabolic or endocrine function, this gene is expressed at moderate levels in pancreas, adipose, adrenal gland, thyroid, pituitary gland, skeletal muscle, heart, liver and the gastrointestinal tract (CTs=27-31). Therefore, therapeutic modulation of the activity of this gene may prove useful in the treatment of endocrine/metabolically related diseases, such as obesity and diabetes.

In addition, this gene is expressed at low to moderate levels (CTs=31-33) in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4D Summary: Ag3539 Highest expression of the CG59307-01 gene is detected in a sample derived from TNFalpha+IL-1beta treated CCD1106 (Keratinocytes) (CT=26.45). Furthermore, expression of this gene is stimulated in activated secondary Th1 cells, LAK cells treated with IL-2/IL-2+IFN gamma/IL-2+IL-12/IL-2+IL-18, LPS treated dendritic cells, monocytes, and macrophages, TNF alpha+IFN gamma treated HUVEC, FNFalpha+IL-1beta treated astrocytes, CCD1106 (Keratinocytes), and lung fibroblasts. Therefore, therapeutics designed with the protein encoded for by this transcript could be important in regulating T cell function and treating T cell mediated diseases such as asthma, arthritis, psoriasis, IBD, cancer cell killing, improvement of host immunity to microbial and viral infections and systemic lupus erythematosus.

### AG. NOV40a (CG59713-01: VAN GOGH)

Expression of gene CG59713-01 was assessed using the primer-probe set Ag3513, described in Table AGA. Results of the RTQ-PCR runs are shown in Tables AGB, AGC and AGD.

TABLE AGA
Probe Name Ag3513

| | | | | Start |
|---|---|---|---|---|
| Primers | Sequences | | Length | Position |
| Forward | 5′-gacttcagcctcgtagtcaatg-3′ | (SEQ ID NO:347) | 22 | 1474 |
| Probe | TET-5′-tgaagaaaattccattcattcatactcttctg-3′-TAMRA | (SEQ ID NO:348) | 30 | 1496 |
| Reverse | 5′-tttgtgagatttggggtctatg-3′ | (SEQ ID NO:349) | 22 | 1533 |

TABLE AGB
CNS_neurodegeneration_v1.0

| | Rel. Exp.(%) Ag3513, | | Rel. Exp.(%) Ag3513, |
|---|---|---|---|
| Tissue Name | Run 210499622 | Tissue Name | Run 210499622 |
| AD 1 Hippo | 32.3 | Control (Path) 3 | 6.9 |
| | | Temporal Ctx | |
| AD 2 Hippo | 46.0 | Control (Path) 4 | 73.2 |
| | | Temporal Ctx | |
| AD 3 Hippo | 12.3 | AD 1 Occipital Ctx | 12.1 |
| AD 4 Hippo | 12.1 | AD 2 Occipital Ctx | 0.0 |
| | | (Missing) | |
| AD 5 hippo | 82.4 | AD 3 Occipital Ctx | 19.6 |
| AD 6 Hippo | 92.7 | AD 4 Occipital Ctx | 22.1 |
| Control 2 Hippo | 20.6 | AD 5 Occipital Ctx | 10.0 |
| Control 4 Hippo | 28.1 | AD 6 Occipital Ctx | 25.5 |
| Control (Path) 3 | 23.2 | Control 1 Occipital | 18.0 |
| Hippo | | Ctx | |
| AD 1 Temporal Ctx | 26.4 | Control 2 Occipital | 44.8 |
| | | Ctx | |
| AD 2 Temporal Ctx | 25.9 | Control 3 Occipital | 21.0 |
| | | Ctx | |
| AD 3 Temporal Ctx | 11.4 | Control 4 Occipital | 21.3 |
| | | Ctx | |
| AD 4 Temporal Ctx | 33.7 | Control (Path) 1 | 100.0 |
| | | Occipital Ctx | |
| AD 5 Inf Temporal | 100.0 | Control (Path) 2 | 16.6 |
| Ctx | | Occipital Ctx | |
| AD 5 Sup Temporal | 100.0 | Control (Path) 3 | 5.9 |
| Ctx | | Occipital Ctx | |
| AD 6 Inf Temporal | 40.6 | Control (Path) 4 | 21.0 |
| Ctx | | Occipital Ctx | |
| AD 6 Sup Temporal | 72.2 | Control 1 Parietal | 13.5 |
| Ctx | | Ctx | |
| Control 1 Temporal | 12.8 | Control 2 Parietal | 49.3 |
| Ctx | | Ctx | |
| Control 2 Temporal | 16.8 | Control 3 Parietal | 11.8 |
| Ctx | | Ctx | |
| Control 3 Temporal | 16.3 | Control (Path) 1 | 50.0 |
| Ctx | | Parietal Ctx | |
| Control 4 Temporal | 10.6 | Control (Path) 2 | 48.0 |
| Ctx | | Parietal Ctx | |
| Control (Path) 1 | 78.5 | Control (Path) 3 | 12.9 |
| Temporal Ctx | | Parietal Ctx | |
| Control (Path) 2 | 22.2 | Control (Path) 4 | 71.2 |
| Temporal Ctx | | Parietal Ctx | |

TABLE AGC
General_screening_panel_v1.4

| | Rel. Exp.(%) Ag3513, | | Rel. Exp.(%) Ag3513, |
|---|---|---|---|
| Tissue Name | Run 217240777 | Tissue Name | Run 217240777 |
| Adipose | 5.6 | Renal ca. TK-10 | 37.4 |
| Melanoma* | 25.7 | Bladder | 17.7 |
| Hs688(A).T | | | |
| Melanoma* | 29.3 | Gastric ca. (liver met.) | 85.3 |
| Hs688(B).T | | NCI-N87 | |
| Melanoma* M14 | 68.8 | Gastric ca. KATO III | 78.5 |
| Melanoma* | 20.3 | Colon ca. SW-948 | 19.3 |
| LOXIMVI | | | |
| Melanoma* SK- | 31.0 | Colon ca. SW480 | 84.1 |
| MEL-5 | | | |
| Squamous cell | 36.1 | Colon ca.* (SW480 | 27.2 |
| carcinoma SCC-4 | | met) SW620 | |
| Testis Pool | 5.3 | Colon ca. HT29 | 26.6 |
| Prostate ca.* (bone | 37.9 | Colon ca. HCT-116 | 73.7 |
| met) PC-3 | | | |
| Prostate Pool | 8.4 | Colon CaCo-2 | 18.4 |
| Placenta | 4.4 | Colon cancer tissue | 13.4 |
| Uterus Pool | 4.7 | Colon ca. SW1116 | 7.9 |
| Ovarian ca. OVCAR-3 | 48.0 | Colon ca. Colo-205 | 17.0 |
| Ovarian ca. SK-OV-3 | 100.0 | Colon ca. SW-48 | 10.6 |
| Ovarian ca. OVCAR-4 | 26.1 | Colon Pool | 18.2 |
| Ovarian ca. OVCAR-5 | 37.1 | Small Intestine Pool | 8.8 |
| Ovarian ca. IGROV-1 | 24.0 | Stomach Pool | 8.9 |
| Ovarian ca. OVCAR-8 | 17.7 | Bone Marrow Pool | 9.2 |
| Ovary | 9.5 | Fetal Heart | 7.0 |
| Breast ca. MCF-7 | 67.4 | Heart Pool | 8.5 |
| Breast ca. MDA- | 67.4 | Lymph Node Pool | 22.1 |
| MB-231 | | | |
| Breast ca. BT 549 | 71.2 | Fetal Skeletal Muscle | 4.0 |
| Breast ca. T47D | 82.9 | Skeletal Muscle Pool | 2.7 |
| Breast ca. MDA-N | 29.7 | Spleen Pool | 5.2 |
| Breast Pool | 16.7 | Thymus Pool | 11.7 |
| Trachea | 14.1 | CNS cancer (glio/astro) | 49.3 |
| | | U87-MG | |
| Lung | 2.4 | CNS cancer (glio/astro) | 34.4 |
| | | U-118-MG | |
| Fetal Lung | 19.5 | CNS cancer (neuro; met) | 19.3 |
| | | SK-N-AS | |
| Lung ca. NCI-N417 | 3.2 | CNS cancer (astro) SF- | 54.7 |
| | | 539 | |
| Lung ca. LX-1 | 17.6 | CNS cancer (astro) | 62.9 |
| | | SNB-75 | |
| Lung ca. NCI-H146 | 9.5 | CNS cancer (glio) SNB-19 | 21.9 |
| | | 19 | |
| Lung ca. SHP-77 | 53.6 | CNS cancer (glio) SF- | 16.0 |
| | | 295 | |
| Lung ca. A549 | 35.6 | Brain (Amygdala) Pool | 1.2 |
| Lung ca. NCI-H526 | 6.8 | Brain (cerebellum) | 1.6 |
| Lung ca. NCI-H23 | 33.4 | Brain (fetal) | 7.2 |
| Lung ca. NCI-H460 | 41.2 | Brain (Hippocampus) | 1.3 |
| | | Pool | |
| Lung ca. HOP-62 | 11.5 | Cerebral Cortex Pool | 1.4 |
| Lung ca. NCI-H522 | 16.5 | Brain (Substantia nigra) | 1.4 |
| | | Pool | |
| Liver | 1.3 | Brain (Thalamus) Pool | 2.2 |
| Fetal Liver | 28.3 | Brain (whole) | 3.8 |
| Liver ca. HepG2 | 21.3 | Spinal Cord Pool | 1.8 |
| Kidney Pool | 24.1 | Adrenal Gland | 16.8 |
| Fetal Kidney | 8.7 | Pituitary gland Pool | 0.5 |
| Renal ca. 786-0 | 55.9 | Salivary Gland | 8.8 |
| Renal ca. A498 | 14.6 | Thyroid (female) | 11.2 |
| Renal ca. ACHN | 17.2 | Pancreatic ca. CAPAN2 | 35.8 |
| Renal ca. UO-31 | 24.0 | Pancreas Pool | 22.8 |

TABLE AGD
Panel 4D

| | Rel. Exp.(%) | | Rel. Exp.(%) |
|---|---|---|---|
| | Ag3513, Run | | Ag3513, Run |
| Tissue Name | 166407114 | Tissue Name | 166407114 |
| Secondary Th1 act | 21.3 | HUVEC IL-1 beta | 17.9 |
| Secondary Th2 act | 26.4 | HUVEC IFN gamma | 18.8 |
| Secondary Tr1 act | 33.7 | HUVEC TNF alpha + IFN | 11.6 |
| | | gamma | |
| Secondary Th1 rest | 11.6 | HUVEC TNF alpha + IL4 | 15.8 |
| Secondary Th2 rest | 8.7 | HUVEC IL-11 | 7.3 |
| Secondary Tr1 rest | 10.2 | Lung Microvascular EC | 18.7 |
| | | none | |
| Primary Th1 act | 6.8 | Lung Microvascular EC | 14.4 |
| | | TNFalpha + IL-1 beta | |
| Primary Th2 act | 20.2 | Microvascular Dermal EC | 26.6 |
| | | none | |
| Primary Tr1 act | 23.2 | Microsvasular Dermal EC | 21.9 |
| | | TNFalpha + IL-1 beta | |
| Primary Th1 rest | 47.0 | Bronchial epithelium | 21.2 |
| | | TNFalpha + IL 1 beta | |
| Primary Th2 rest | 18.3 | Small airway epithelium | 13.8 |
| | | none | |
| Primary Tr1 rest | 16.4 | Small airway epithelium | 89.5 |
| | | TNFalpha + IL-1 beta | |
| CD45RA CD4 | 10.2 | Coronery artery SMC rest | 10.5 |
| lymphocyte act | | | |
| CD45RO CD4 | 19.2 | Coroncry artery SMC | 8.4 |
| lymphocyte act | | TNFalpha + IL-1 beta | |
| CD8 lymphocyte act | 4.4 | Astrocytes rest | 15.3 |
| Secondary CD8 | 6.9 | Astrocytes TNFalpha + IL- | 23.3 |
| lympnocyte rest | | 1 beta | |
| Secondary CD8 | 11.0 | KU-812 (Basophil) rest | 21.2 |
| lymphocyte act | | | |
| CD4 lymphocyte none | 1.4 | KU-812 (Basophil) | 34.6 |
| | | PMA/ionomycin | |
| 2ry Th1/Th2/Tr1_anti- | 13.2 | CCD1106 (Keratinocytes) | 32.5 |
| CD95 CH11 | | none | |
| LAK cells rest | 2.1 | CCD1106 (Keratinocytes) | 100.0 |
| | | TNFalpha + IL-1 beta | |
| LAK cells IL-2 | 37.4 | Liver cirrhosis | 2.9 |
| LAK cells IL-2 + IL-12 | 12.4 | Lupus kidney | 5.6 |
| LAK cells IL-2 + IFN | 18.7 | NCI-H292 none | 36.6 |
| gamma | | | |
| LAK cells IL-2 + IL-18 | 17.7 | NCI-H292 IL-4 | 69.3 |
| LAK cells | 0.6 | NCI-H292 IL-9 | 56.6 |
| PMA/ionomycin | | | |
| NK Cells IL-2 rest | 21.5 | NCI-H292 IL-13 | 35.4 |
| Two Way MLR 3 day | 3.8 | NCI-H292 IFN gamma | 29.3 |
| Two Way MLR 5 day | 5.6 | HPAEC none | 12.0 |
| Two Way MLR 7 day | 5.1 | HPAEC TNF alpha + IL-1 | 10.8 |
| | | beta | |
| PBMC rest | 2.6 | Lung fibroblast none | 11.2 |
| PBMC PWM | 4.5 | Lung fibroblast TNF | 13.9 |
| | | alpha + IL-1 beta | |
| PBMC PHA-L | 4.1 | Lung fibroblast IL-4 | 16.5 |
| Ramos (B cell) none | 0.2 | Lung fibroblast IL-9 | 11.4 |
| Ramos (B cell) | 0.0 | Lung fibroblast IL-13 | 8.6 |
| ionomycin | | | |
| B lymphocytes PWM | 11.3 | Lung fibroblast IFN | 19.8 |
| | | gamma | |
| B lymphocytes CD40L | 4.9 | Dermal fibroblast | 44.8 |
| and IL-4 | | CCD 1070 rest | |
| EOL-1 dbcAMP | 5.5 | Dermal fibroblast | 49.7 |
| | | CCD1070 TNF alpha | |
| EOL-1 dbcAMP | 3.5 | Dermal fibroblast | 19.6 |
| PMA/ionomyein | | CCD1070 IL-1 beta | |
| Dendritic cells none | 2.9 | Dermal fibroblast IFN | 11.7 |
| | | gamma | |
| Dendritic cells LPS | 1.7 | Dermal fibroblast IL-4 | 21.5 |
| Dendritic cell anti- | 3.6 | IBD Colitis 2 | 1.2 |
| CD 40 | | | |
| Monocytes rest | 1.1 | IBD Crohn's | 1.8 |
| Monocytes LPS | 0.4 | Colon | 23.0 |
| Macrophages rest | 5.6 | Lung | 9.4 |
| Macrophages LPS | 1.6 | Thymus | 16.2 |
| HUVEC none | 35.6 | Kidney | 16.2 |
| HUVEC starved | 40.3 | | |

CNS_neurodegeneration_v1.0 Summary: Ag3513 This panel confirms the expression of the CG59713-01 gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3513 Highest expression of the CG59713-01 is detected in sample derived from one of the ovarian cancer SK-OV-3(CT=28). Furthermore, significant expression of this gene is associated with number of cancer samples (pancreatic cancer, CNS cancer, colon cancer, gastric cancer, renal cancer, lung cancer, breast cancer, prostate cancer, melanoma cell lines) used in this panel. Therefore, therapeutic modulation of the activity of this gene or its protein product, through the use of small molecule drugs, or antibodies, might be beneficial in the treatment of all these cancers.

Interestingly, this gene is expressed at much higher levels in fetal (CT=30) when compared to adult liver (CT=34). This observation suggests that expression of this gene can be used to distinguish fetal from adult liver. In addition, the relative overexpression of this gene in fetal liver suggests that the protein product may be required for growth and development of the liver in fetus and thus may also act in a regenerative capacity in the adult. Therefore, therapeutic modulation of the protein encoded by this gene could be useful in treatment of liver related diseases.

Among tissues with metabolic or endocrine function, this gene is expressed at high to moderate levels (CTs=30-32) in pancreas, adipose, adrenal gland, thyroid, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at low levels (CTs=32-34) in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4D Summary: Ag3513 Highest expression of the CG59713-01 is detected in TNFalpha+IL-1beta treated keratinocytes (CT=27.9). Therefore, expression of this gene can be used to distinguish TNFalpha+IL-1beta treated keratinocytes from other samples used in this panel.

In addition, expression of this gene is stimulated in activated secondary Th1, Th2, and Tr1 cells, LAK cells treated with IL-2, TNFalpha+IL-1beta treated small airway epithelium and NCI-H292 treated with IL-4. Therefore, therapeutic modulation of the activity of this gene or its protein product, through the use of small molecule drugs, or antibodies, might be beneficial in the treatment of psoriasis, asthma, emphysema, cancer, microbial and viral infections.

### AH. NOV41a (CG59570-01: AQUAPORIN)

Expression of gene CG59570-01 was assessed using the primer-probe set Ag3475, described in Table AHA. Results of the RTQ-PCR runs are shown in Tables AHB, and AHC.

TABLE AHA
Probe Name Ag3475

| | | | | Start |
|---|---|---|---|---|
| Primers | Sequences | | Length | Positon |
| Forward | 5′-acaaaacatggttcaagcatct-3′ | (SEQ ID NO:350) | 22 | 2 |
| Probe | TET-5′-ccaaaatggtctcctggtccgtgata-3′-TAMRA | (SEQ ID NO: 351) | 26 | 49 |
| Reverse | 5′-accatatttcctggatctttgc-3′ | (SEQ ID NO:352) | 22 | 75 |

TABLE AHB
General_screening_panel_v1.4

| | Rel. Exp.(%) Ag3475, | | Rel. Exp.(%) Ag3475, |
|---|---|---|---|
| Tissue Name | Run 222691300 | Tissue Name | Run 222691300 |
| Adipose | 100.0 | Renal ca. TK-10 | 1.1 |
| Melanoma* | 0.0 | Bladder | 3.4 |
| Hs688(A).T | | | |
| Melanoma* | 0.1 | Gastric ca. (liver met.) | 8.5 |
| Hs688(B).T | | NCI-N87 | |
| Melanoma* M14 | 0.2 | Gastric ca. KATO III | 0.2 |
| Melanoma* | 1.1 | Colon ca. SW-948 | 1.2 |
| LOXIMVI | | | |
| Melanoma* SK- | 0.0 | Colon ca. SW480 | 0.0 |
| MEL-5 | | | |
| Squamous cell | 0.0 | Colon ca.* (SW480 | 0.4 |
| carcinoma SCC-4 | | met) SW620 | |
| Testis Pool | 10.2 | Colon ca. HT29 | 0.2 |
| Prostate ca.* (bone | 2.2 | Colon HCT-116 | 0.6 |
| met) PC-3 | | | |
| Prostate Pool | 0.6 | Colon ca. CaCo-2 | 0.0 |
| Placenta | 0.3 | Colon cancer tissue | 9.6 |
| Uterus Pool | 0.8 | Colon ca. SW1116 | 0.0 |
| Ovarian ca. OVCAR-3 | 0.5 | Colon ca. Colo-205 | 0.0 |
| Ovarian ca. SK-OV-3 | 0.1 | Colon ca. SW-48 | 0.0 |
| Ovarian ca. OVCAR-4 | 0.3 | Colon Pool | 1.5 |
| Ovarian ca. OVCAR-5 | 0.3 | Small Intestine Pool | 0.6 |
| Ovarian ca. IGROV-1 | 5.0 | Stomach Pool | 2.2 |
| Ovarian ca. OVCAR-8 | 0.3 | Bone Marrow Pool | 0.8 |
| Ovary | 0.1 | Fetal Heart | 1.2 |
| Breast ca. MCF-7 | 0.1 | Heart Pool | 20.6 |
| Breast ca. MDA- | 0.1 | Lymph Node Pool | 1.3 |
| MB-231 | | | |
| Breast ca. BT 549 | 0.3 | Fetal Skeletal Muscle | 4.7 |
| Breast ca. T47D | 0.5 | Skeletal Muscle Pool | 14.3 |
| Breast ca. MDA-N | 0.5 | Spleen Pool | 1.9 |
| Breast Pool | 1.0 | Thymus Pool | 6.0 |
| Trachea | 4.0 | CNS cancer (glio/astro) | 0.7 |
| | | U87-MG | |
| Lung | 0.2 | CNS cancer (glio/astro) | 4.7 |
| | | U-118-MG | |
| Fetal Lung | 0.4 | CNS cancer (neuro; met) | 0.2 |
| | | SK-N-AS | |
| Lung ca. NCI-N417 | 0.0 | CNS cancer (astro) SF- | 0.0 |
| | | 539 | |
| Lung ca. LX-1 | 0.7 | CNS cancer (astro) | 2.8 |
| | | SNB-75 | |
| Lung ca. NCI-H146 | 0.0 | CNS cancer (glio) SNB- | 3.8 |
| | | 19 | |
| Lung ca. SHP-77 | 0.0 | CNS cancer (glio) SF- | 0.2 |
| | | 295 | |
| Lung ca. A549 | 0.2 | Brain (Amygdala) Pool | 0.7 |
| Lung ca. NCI-H526 | 0.0 | Brain (cerebellum) | 13.2 |
| Lung ca. NCI-H23 | 0.1 | Brain (fetal) | 0.1 |
| Lung ca. NCI-H460 | 0.3 | Brain (Hippocampus) | 1.3 |
| | | Pool | |
| Lung ca. HOP-62 | 0.3 | Cerebral Cortex Pool | 0.1 |
| Lung ca. NCI-H522 | 1.7 | Brain (Substantia nigra) | 0.8 |
| | | Pool | |
| Liver | 0.0 | Brain (Thalamus) Pool | 0.1 |
| Fetal Liver | 0.0 | Brain (whole) | 0.2 |
| Liver ca. HepG2 | 2.0 | Spinal Cord Pool | 1.3 |
| Kidney Pool | 3.6 | Adrenal Gland | 10.2 |
| Fetal Kidney | 0.9 | Pituitary gland Pool | 0.0 |
| Renal ca.786-0 | 1.3 | Salivary Gland | 2.5 |
| Renal ca. A498 | 0.5 | Thyroid (female) | 0.7 |
| Renal ca. ACHN | 0.0 | Pancreatic ca. CAPAN2 | 0.0 |
| Renal ca. UO-31 | 0.1 | Pancreas Pool | 1.1 |

TABLE AHC
Panel 4D

| | Rel. Exp. (%) | | Rel. Exp. (%) |
|---|---|---|---|
| | Ag3475, Run | | Ag3475, Run |
| Tissue Name | 166420470 | Tissue Name | 166420470 |
| Secondary Th1 act | 1.9 | HUVEC IL-1 beta | 1.6 |
| Secondary Th2 act | 0.7 | HUVEC IFN gamma | 3.5 |
| Secondary Tr1 act | 0.9 | HUVEC TNF alpha + IFN | 0.5 |
| | | gamma | |
| Secondary Th1 rest | 1.5 | HUVEC TNF alpha + IL4 | 2.0 |
| Secondary Th2 rest | 1.5 | HUVEC IL-11 | 3.1 |
| Secondary Tr1 rest | 0.6 | Lung Microvascular EC | 50.3 |
| | | none | |
| Primary Th1 act | 0.0 | Lung Microvascular EC | 22.1 |
| | | TNFalpha + IL-1beta | |
| Primary Th2 act | 1.2 | Microvascular Dermal EC | 0.8 |
| | | none | |
| Primary Tr1 act | 1.0 | Microvascular Dermal EC | 1.3 |
| | | THFalpha + IL-1beta | |
| Primary Th1 rest | 3.3 | Bronchial epithelium | 0.6 |
| | | TNFalpha + IL1beta | |
| Primary Th2 rest | 2.2 | Small airway epithelium | 2.7 |
| | | none | |
| Primary Tr1 rest | 1.1 | Small airway epithelium | 17.8 |
| | | TNFalpha + IL-1beta | |
| CD45RA CD4 | 0.0 | Coronery artery SMC rest | 1.7 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 2.6 |
| Secondary CD8 | 1.4 | Astrocytes TNFalpha + IL- | 6.6 |
| lymphocyte rest | | 1 beta | |
| Secondary CD8 | 0.0 | KU-812 (Basophil) rest | 0.0 |
| lymphocyte act | | | |
| CD4 lymphocyte none | 0.0 | KU-812 (Basophil) | 1.0 |
| | | PMA/ionomycin | |
| 2ry TH1/TH2/TR1_anti- | 5.1 | CCD1106 (Keratinocytes) | 1.2 |
| CD95 CH11 | | none | |
| LAK cells rest | 0.0 | CCD1106 (Keratinocytes) | 3.1 |
| | | TNFalpha + IL-1beta | |
| LAK cells IL-2 | 0.0 | Liver cirrhosis | 4.5 |
| LAK cells IL-2 + IL-12 | 0.5 | Lupus kidney | 13.4 |
| LAK cells IL-2 + IFN | 0.0 | NCI-H292 none | 3.0 |
| gamma | | | |
| LAK cells IL-2 + IL-18 | 0.3 | NCI-H292 IL-4 | 0.8 |
| LAK cells | 0.7 | NCI-H292 IL-9 | 4.3 |
| PMA/ionomycin | | | |
| NK Cells IL-2 rest | 1.0 | NCI-H292 IL-13 | 3.3 |
| Two Way MLR 3 day | 0.0 | NCI-H292 IFN gamma | 1.7 |
| Two Way MLR 5 day | 0.0 | HPAEC none | 100.0 |
| Two Way MLR 7 day | 0.6 | HPAEC TNF alpha + IL-1 | 78.5 |
| | | beta | |
| PBMC rest | 0.6 | Lung fibroblast none | 5.8 |
| PBMC PWM | 0.0 | Lung fibroblasts TNF | 2.7 |
| | | alpha + IL-1 beta | |
| PBMC PHA-L | 0.0 | Lung fibroblast IL-4 | 3.8 |
| Ramos (B cell) none | 0.2 | Lung fibroblast IL-9 | 1.3 |
| Ramos (B cell) | 0.0 | Lung fibroblast IL-13 | 0.3 |
| ionomycin | | | |
| B lymphocytes PWM | 0.0 | Lung fibroblast IFN | 3.5 |
| | | gamma | |
| B lymphocytes CD40L | 0.0 | Dermal fibroblast | 3.1 |
| and IL-4 | | CCD1070 rest | |
| EOL-1 dbcAMP | 0.0 | Dermal fibroblast | 2.0 |
| | | CCD1070 TNF alpha | |
| EOL-1 dbcAMP | 0.4 | Dermal fibroblast | 0.0 |
| PMA/ionomycin | | CCD1070 IL-1 beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 1.8 | IBD Colitis 2 | 2.5 |
| CD40 | | | |
| Monocytes rest | 0.0 | IBD Crohn's | 12.3 |
| Monocytes LPS | 0.0 | Colon | 25.7 |
| Macrophages rest | 0.0 | Lung | 1.3 |
| Macrophages LPS | 0.0 | Thymus | 38.4 |
| HUVEC none | 3.1 | Kidney | 20.9 |
| HUVEC starved | 4.5 | | |

CNS_neurodegeneration_v1.0 Summary: Ag3475 Expression of the CG59570-01 gene is low/undetectable (CTs>34.5) across all of the samples on this panel.

General_screening_panel_v1.4 Summary: Ag3475 High expression of the CG59570-01 gene is detected exclusively in adipose tissue sample (CT=27.73). Thus expression of this gene can be used to distinguish adipose sample from other samples in this panel. In addition to adipose tissue, low levels of expression of this gene is also seen in other tissues with metabolic or endocrine function such as pancreas, adrenal gland, thyroid, skeletal muscle, heart, and the gastrointestinal tract. Therefore, therapeutic modulation of the activity of this gene may prove useful in the treatment of endocrine/metabolically related diseases, such as obesity and diabetes.

CG59570-01 codes for protein similar to aquaporin 7. Aquaporins are water channels that are usually found in tissues where water movements are abundant and/or physiologically important. In a systematic analysis of genes expressed in human adipose tissue, Kuniyama et al. (Ref. 1) identified AQP7L, a predicted 342-amino acid protein. They showed that this aquaporin participates in glycerol transport in adipocytes. Thus, we predict that protein encoded by this transcript may also play a role in glycerol transport in adipocytes.

Interestingly, this gene is expressed at much higher levels in adult (CT=30) when compared to fetal heart (CT=34). This observation suggests that expression of this gene can be used to distinguish fetal from adult heart. Furthermore, therapeutic modulation of the protein encoded by this gene could be useful in treatment of heart related diseases.

In addition, this gene is expressed at significant levels in some regions of the central nervous system examined, including amygdala, hippocampus, substantia nigra, and cerebellum (CTs=30-34). Therefore, this gene may play a role in central nervous system disorders such as Alzheimer's disease, Parkinson's disease, epilepsy, seizure, ataxia, autism, schizophrenia and depression.

Reference.

1. Kuriyama, H.; Kawamoto, S.; Ishida, N.; Ohno, I.; Mita, S.; Matsuzawa, Y.; Matsubara, K.; Okubo, K. (1997) Molecular cloning and expression of a novel human aquaporin from adipose tissue with glycerol permeability. Biochem. Biophys. Res. Commun. 241: 53-58.

Panel 4D Summary: Ag3475 High expression of the CG59570-01 is detected untreated and TNF alpha+IL-1beta treated (CTs=30) HPAEC cells. Thus expression of this gene can be used to distinguish these two samples from other samples in this panel. In addition, low levels of expression of this gene is also detected in lung fibroblast, IFN-gamma treated lung fibroblast, IL-9 treated NCI-H292 cells, starved and IFN-gama treated HUVEC cells, anti-CD95 CH11 treated secondary Th1/Th2/Tr1 cells, and TNFalpha+IL-1beta treated small airway epithelium. Therefore, therapeutic modulation of the activity of this gene may prove useful in the treatment of lung related diseases such as asthma, allergies, emphysema, COPD.

Interestingly, this gene is expressed at much higher levels in TNFalpha+IL-1beta treated (CT=32) as compared to untreated small airway epithelium (CT=35). Thus, expression of this gene can be used to distinguish TNFalpha+IL-1beta treated from the untreated small airway epithelium cells.

Expression of this gene is detected at low levels (CT=34.5) in liver cirrhosis, but not in normal liver (no expression in normal liver is detected on Panel 1.4). The protein encoded for by this gene could potentially allow cells within the liver to respond to specific microenvironmental signals. Therefore, therapies designed with the protein encoded for by this gene may potentially modulate liver function and play a role in the identification and treatment of inflammatory or autoimmune diseases which effect the liver including liver cirrhosis and fibrosis.

In addition, moderate expression of this gene is also observed in kidney, thymus and colon. Therefore, antibody or small molecule therapies designed with the protein encoded for by this gene may be important in the treatment of inflammatory or autoimmune diseases that affect these tissues.

### AI. NOV42a (CG56162-02: LYSOPHOSPHOLIPASE-LIKE)

Expression of gene CG56162-02 was assessed using the primer-probe set Ag2042, described in Table AIA. Results of the RTQ-PCR runs are shown in Tables AIB and AIC. Please note that CG56162-02 represents a full-length physical clone.

TABLE AIA
Probe Name Ag2042

| | | | | Start |
|---|---|---|---|---|
| Primers | Sequences | | Length | Position |
| Forward | 5′-cataatggaaacaggacctgaa-3′ | (SEQ ID NO:353) | 22 | 32 |
| Probe | TET-5′-ccttccagcatgccagaggaaagtt-3′-TAMRA | (SEQ ID NO:354) | 25 | 57 |
| Reverse | 5′-aggtcctggtagggaatgct-3′ | (SEQ ID NO:355) | 20 | 102 |

TABLE AIB
Panel 1.3D

| | Rel. Exp. (%) Ag 2042, | | Rel. Exp. (%) Ag2042, |
|---|---|---|---|
| Tissue Name | Run 165627321 | Tissue Name | Run 165627321 |
| Liver adenocarcinoma | 5.8 | Kidney (fetal) | 6.4 |
| Pancreas | 0.8 | Renal ca. 786-0 | 25.7 |
| Pancreatic ca. CAPAN | 8.5 | Renal ca. A498 | 27.0 |
| 2 | | | |
| Adrenal gland | 1.6 | Renal ca. RXF 393 | 46.0 |
| Thyroid | 1.4 | Renal ca. ACHN | 14.7 |
| Salivary gland | 6.2 | Renal ca. UO-31 | 41.8 |
| Pituitary gland | 1.6 | Renal ca. TK-10 | 8.5 |
| Brain (fetal) | 33.7 | Liver | 3.5 |
| Brain (whole) | 92.0 | Liver (fetal) | 0.9 |
| Brain (amygdala) | 37.9 | Liver ca. | 0.0 |
| | | (hepatoblast) HepG2 | |
| Brain (cerebellum) | 94.6 | Lung | 8.8 |
| Brain (hippocampus) | 50.7 | Lung (fetal) | 12.1 |
| Brain (subtantia nigra) | 11.6 | Lung ca. (small cell) | 2.1 |
| | | LX-1 | |
| Brain (thalamus) | 51.1 | Lung ca. (small cell) | 6.1 |
| | | NCI-H69 | |
| Cerebral Cortex | 76.3 | Lung ca. (s.cell var.) | 0.0 |
| | | SHP-77 | |
| Spinal cord | 12.0 | Lung ca. (large | 1.6 |
| | | cell) NCI-H460 | |
| glio/astro U87-MG | 28.3 | Lung ca. (non-sm. | 6.4 |
| | | cell) A549 | |
| glio/astro U-118-MG | 100.0 | Lung ca. (non-s.cell) | 0.4 |
| | | NCI-H23 | |
| astrocytoma SW1783 | 3.2 | Lung ca. (non-s.cell) | 0.0 |
| | | HOP-62 | |
| neuro*; met SK-N-AS | 0.3 | Lung ca. (non-s.cl) | 0.0 |
| | | NCI-H522 | |
| astrocytoma SF-539 | 40.9 | Lung ca. (squam.) | 12.6 |
| | | SW 900 | |
| astrocytoma SNB-75 | 12.3 | Lung ca. (squam.) | 9.0 |
| | | NCI-H596 | |
| glioma SNB-19 | 4.1 | Mammary gland | 9.5 |
| glioma U251 | 1.5 | Breast ca.* (pl.ef) | 5.8 |
| | | MCF-7 | |
| glioma SF-295 | 1.4 | Breast ca.* (pl.ef) | 21.9 |
| | | MDA-MB-231 | |
| Heart (fetal) | 6.0 | Breast ca.* (pl.ef) | 0.4 |
| | | T47D | |
| Heart | 12.8 | Breast ca. BT-549 | 0.3 |
| Skeletal muscle (fetal) | 8.5 | Breast ca. MDA-N | 1.3 |
| Skeletal muscle | 6.2 | Ovary | 2.1 |
| Bone marrow | 2.3 | Overian ca. OVCAR- | 0.0 |
| | | 3 | |
| Thymus | 1.0 | Ovarian ca. OVCAR- | 7.6 |
| | | 4 | |
| Spleen | 13.3 | Ovarian ca. OVCAR- | 10.2 |
| | | 5 | |
| Lymph node | 5.7 | Ovarian ca. OVCAR- | 10.6 |
| | | 8 | |
| Colorectal | 10.5 | Ovarian ca. IGROV-1 | 3.2 |
| Stomach | 5.5 | Ovarian ca.* (ascites) | 45.7 |
| | | SK-OV-3 | |
| Small intestine | 10.2 | Uterus | 8.7 |
| Colon ca. SW480 | 0.3 | Placenta | 4.3 |
| Colon ca.* | 0.0 | Prostate | 1.3 |
| SW620 (SW480 met) | | | |
| Colon ca. HT29 | 8.1 | Prostate ca.* (bone | 22.2 |
| | | met) PC-3 | |
| Colon ca. HCT-116 | 2.4 | Testis | 0.8 |
| Colon ca. CaCo-2 | 0.8 | Melanoma | 1.4 |
| | | Hs688(A).T | |
| Colon ca. | 10.8 | Melanoma* (met) | 4.5 |
| tissue (ODO3866) | | Hs688(B).T | |
| Colon ca. HCC-2998 | 2.2 | Melanonma UACC-62 | 3.0 |
| Gastric ca.* (liver met) | 7.3 | Melanoma M14 | 0.0 |
| NCI-N87 | | | |
| Bladder | 1.5 | Melanoma LOX | 1.4 |
| | | IMVI | |
| Trachea | 11.6 | Melanoma* (met) | 0.1 |
| | | SK-MEL-5 | |
| Kidney | 5.1 | Adipose | 9.2 |

TABLE AIC
Panel 4D

| | Rel. Exp. (%) | | Rel. Exp. (%) |
|---|---|---|---|
| | Ag2042, Run | | Ag2042, Run |
| Tissue Name | 161383006 | Tissue Name | 161383006 |
| Secondary TH1 act | 0.3 | HUVEC IL-1beta | 22.8 |
| Secondary Th2 act | 0.7 | HUVEC IFN gamma | 17.4 |
| Secondary Tr1 act | 1.0 | HUVEC TNF alpha + IFN | 59.0 |
| | | gamma | |
| Secondary Th1 rest | 0.0 | HUVEC TNF alpha + IL4 | 54.0 |
| Secondary Th2 rest | 1.5 | HUVEC IL-11 | 14.1 |
| Secondary Tr1 rest | 0.9 | Lung Microvascular EC | 42.0 |
| | | none | |
| Primary Th1 act | 0.9 | Lung Microvascular EC | 79.6 |
| | | TNFalpha + IL-1beta | |
| Primary Th2 act | 1.9 | Microvascular Dermal EC | 54.3 |
| | | none | |
| Primary Tr1 act | 0.4 | Microvascular Dermal EC | 54.3 |
| | | TNFalpha + IL-1beta | |
| Primary Th1 rest | 1.2 | Bronchial epithelium | 7.8 |
| | | TNFalpha + IL1beta | |
| Primary Th2 rest | 1.5 | Small airway epithelium | 12.8 |
| | | none | |
| Primary Tr1 rest | 0.8 | Small airway epithelium | 41.5 |
| | | TNFalpha + IL-1beta | |
| CD45RA CD4 | 15.3 | Coronery artery SMC rest | 0.0 |
| lymphocyte act | | | |
| CD45RO CD4 | 0.4 | Coronery artery SMC | 7.3 |
| lymphocyte act | | TNFalpha + IL-1beta | |
| CD8 lymphocyte act | 1.2 | Astrocytes rest | 0.7 |
| Secondary CD8 | 0.2 | Astrocytes TNFalpha + IL- | 3.6 |
| lymphocyte rest | | 1beta | |
| Secondary CD8 | 3.0 | KU-812 (Basophil) rest | 0.0 |
| lymphocyte act | | | |
| CD4 lymphocyte none | 0.1 | KU-812 (Basophil) | 0.3 |
| | | PMA/ionomycin | |
| 2ry Th1/Th2/Tr1_anti- | 1.3 | CCD1106 (Keratinocytes) | 18.0 |
| CD95 CH11 | | none | |
| LAK cells rest | 3.3 | CCD1106 (Keratinocytes) | 1.0 |
| | | TNFalpha + IL-1beta | |
| LAK cells IL-2 | 0.2 | Liver cirrhosis | 3.5 |
| LAK cells IL-2 + IL-12 | 1.2 | Lupus kidney | 5.4 |
| LAK cells IL-2 + IFN | 3.2 | NCI-H292 none | 16.3 |
| gamma | | | |
| LAK cells IL-2 + IL-18 | 1.7 | NCI-H292 IL-4 | 24.1 |
| LAK cells | 2.1 | NCI-H292 IL-9 | 23.3 |
| PMA/ionomycin | | | |
| NK Cells IL-2 rest | 0.0 | NCI-H292 IL-13 | 11.9 |
| Two Way MLR 3 day | 7.3 | NCI-H292 IFN gamma | 21.9 |
| Two Way MLR 5 day | 2.5 | HPAEC none | 25.7 |
| Two Way MLR 7 day | 1.3 | HPAEC TNF alpha + IL-1 | 64.6 |
| | | beta | |
| PBMC rest | 0.4 | Lung fibroblast none | 18.8 |
| PBMC PWM | 9.2 | Lung fibroblast TNF | 33.7 |
| | | alpha + IL-1 beta | |
| PBMC PHA-L | 2.4 | Lung fibroblast IL-4 | 29.5 |
| Ramos (B cell) none | 0.0 | Lung fibroblast IL-9 | 43.2 |
| Ramos (B cell) | 0.0 | Lung fibroblast IL-13 | 18.7 |
| ionomycin | | | |
| B lymphocytes PWM | 10.7 | Lung fibroblast IFN | 29.1 |
| | | gamma | |
| B lymphocytes CD40L | 8.3 | Dermal fibroblast | 64.6 |
| and IL-4 | | CCD1070 rest | |
| EOL-1 dbcAMP | 0.0 | Dermal fibroblast | 100.0 |
| | | CCD1070 TNF alpha | |
| EOL-1 dbcAMP | 0.1 | Dermal fibroblast | 48.0 |
| PMA/ionomycin | | CCD1070 IL-1 beta | |
| Dendritic cells none | 7.3 | Dermal fibroblast IFN | 24.0 |
| | | gamma | |
| Dendritic cells LPS | 28.1 | Dermal fibroblast IL-4 | 41.5 |
| Dendritic cells anti- | 13.0 | IBD Colitis 2 | 0.4 |
| CD40 | | | |
| Monocytes rest | 0.1 | IBD Crohn's | 1.0 |
| Monocytes LPS | 16.5 | Colon | 18.4 |
| Macrophages rest | 51.4 | Lung | 44.1 |
| Macrophages LPS | 18.0 | Thymus | 17.2 |
| HUVEC none | 45.1 | Kidney | 5.4 |
| HUVEC starved | 54.7 | | |

Panel 1.3D Summary: Ag1906/Ag2042 The expression of CG56162-02 gene was assessed in two independent runs using 2 different probe/primer pairs, with good concordance between the runs. Highest expression in this panel is seen in brain derived tissue, including the cerebral cortex and a brain cancer (CTs=25-27). Thus, the expression of this gene could be used to distinguish these samples from other samples in the panel.

This gene encodes a lysophosphase homolog that also has high levels of expression in many of the endocrine/metabolic tissues found on this panel, including adipose, liver, pancreas, pituitary, skeletal muscle and small intestine. Lysophospholipids are detergent-like intermediates in phospholipid metabolism. Lysophospholipases are important enzymes in the regulation of hormone biosynthesis and metabolism, and have been shown to be important in the regulation of insulin secretion (see reference below). Increased lysophospholipids levels have been detected in a variety of diseases including atherosclerosis and hyperlipidemia. In some cases, increased levels of lysophospholipids are hypothesized to result from a dysfunction of lysophospholipids-regulating enzymes including lysophospholipases, which act on biologic membranes to regulate the level of lysophospholipids by hydrolysis. Thus, this gene product may be useful in the treatment of diseases associated with increased lysophospholipids.

This gene also shows high expression in the brain. Lysophospholipases are critical enzymes that regulate brain membrane phospholipids. Alterations in their activity have been associated with a host of neurological disorders, including schizophrenia, Parkinson's disease, and Alzheimer's disease. Thus, therapeutic modulation of the expression or function of this gene or gene product may be useful in the treatment of these diseases. Please note that results from a third experiment with the probe/primer set Ag1952 are not included. The amp plot indicates that there were experimental difficulties with this run.

References:

1. Capito K, Reinsmark R, Thams P. Mechanism of fat-induced attenuation of glucose-induced insulin secretion from mouse pancreatic islets. Acta Diabetol December 1999;36(3): 119-25

2. Ross B M, Turenne S, Moszczynska A, Warsh J J, Kish S J. Differential alteration of phospholipase A2 activities in brain of patients with schizophrenia. Brain Res Mar. 13, 1999; 821 (2):407-13

3. Ross B M, Moszczynska A, Erlich J, Kish S J. Low activity of key phospholipid catabolic and anabolic enzymes in human substantia nigra: possible implications for Parkinson's disease. Neuroscience April 1998;83(3):791-8

4. Ross B M, Moszczynska A, Erlich J, Kish S J. Phospholipid-metabolizing enzymes in Alzheimer's disease: increased lysophospholipid acyltransferase activity and decreased phospholipase A2 activity. J Neurochem February 1998;70(2):786-93

Panel 4D Summary: Ag1906/Ag1952/Ag2042 The expression ofCG56162-02 gene was assessed in three independent runs using three different probe/primer pairs, with good concordance between the runs. This gene is expressed at moderate levels in a wide variety of cells including resting macrophages, TNF-alpha-activated dermal fibroblasts, LPS-stimulated dendritic cells, TN-alpha+IL-1-beta-activated pulmonary artery endothelial cells, TNF-alpha+IL-1-beta-activated lung microvascular cells, and TNF-alpha+IFN-gamma-activated umbilical vein endothelial cells (CTs=27-28). Thus, antibodies and small molecules that antagonize the function of the CG120803-01 geen product may be useful to reduce or eliminate the symptoms in patients with inflammatory and autoimmune diseases, such as lupus erythematosus, asthma, emphysema, Crohn's disease, ulcerative colitis, multiple sclerosis, rheumatoid arthritis, osteoarthritis, and psoriasis.

### AJ. NOV45a and NOV45b (CG59859-01 and CG59859-02: TESTIS EXPRESSED PROTEIN 261 (TEG-261))

Expression of genes CG59859-01 and CG59859-02 was assessed using the primer-probe set Ag3623, described in Table AJA. Results of the RTQ-PCR runs arc shown in Tables AJB, AJC and AJD. Please note that CG59859-02 represents a full-length physical clone of the CG59859-01 gene, validating the prediction of the gene sequence.

TABLE AJA
Probe Name Ag3623

| | | | | Start |
|---|---|---|---|---|
| Primers | Sequences | | Length | Position |
| Forward | 5′-cgtctccaattatttcaccaaa-3′ | (SEQ ID NO:356) | 22 | 534 |
| Probe | TET-5′-agggatccttggttgtcttctccttca-3′-TAMRA | (SEQ ID NO:357) | 26 | 576 |
| Reverse | 5′-cttctgacgactgggtagaatg-3′ | (SEQ ID NO:358) | 22 | 612 |

TABLE AJB
CNS_neurodegeneration_v1.0

| | Rel. Exp. (%) Ag3623, | | Rel. Exp. (%) Ag2623, |
|---|---|---|---|
| Tissue Name | Run 211005294 | Tissue Name | Run 211005294 |
| AD 1 Hippo | 22.4 | Control (Path) 3 | 12.2 |
| | | Temporal Ctx | |
| AD 2 Hippo | 42.9 | Control (Path) 4 | 25.9 |
| | | Temporal Ctx | |
| AD 3 Hippo | 16.6 | AD 1 Occipital Ctx | 21.6 |
| AD 4 Hippo | 9.5 | AD 2 Occipital Ctx | 0.0 |
| | | (Missing) | |
| AD 5 hippo | 76.3 | AD 3 Occipital Ctx | 21.2 |
| AD 6 Hippo | 50.7 | AD 4 Occipital Ctx | 22.7 |
| Control 2 Hippo | 32.8 | AD 5 Occipital Ctx | 5.8 |
| Control 4 Hippo | 28.5 | AD 6 Occipital Ctx | 39.0 |
| Control (Path) 3 | 9.8 | Control 1 Occipital | 14.3 |
| Hippo | | Ctx | |
| AD 1 Temporal Ctx | 27.7 | Control 2 Occipital | 80.1 |
| | | Ctx | |
| AD 2 Temporal Ctx | 48.0 | Control 3 Occipital | 18.8 |
| | | Ctx | |
| AD 3 Temporal Ctx | 15.4 | Control 4 Occipital | 13.9 |
| | | Ctx | |
| AD 4 Temporal Ctx | 13.9 | Control (Path) 1 | 100.0 |
| | | Occipital Ctx | |
| AD 5 Inf Temporal | 86.5 | Control (Path) 2 | 15.3 |
| Ctx | | Occipital Ctx | |
| AD 5 Sup Temporal | 64.6 | Control (Path) 3 | 9.4 |
| Ctx | | Occipital Ctx | |
| AD 6 Inf Temporal | 62.4 | Control (Path) 4 | 18.9 |
| Ctx | | Occipital Ctx | |
| AD 6 Sup Temporal | 63.7 | Control 1 Parietal | 17.8 |
| Ctx | | Ctx | |
| Control 1 Temporal | 14.1 | Control 2 Parietal | 40.1 |
| Ctx | | Ctx | |
| Control 2 Temporal | 40.1 | Control 3 Parietal | 16.2 |
| Ctx | | Ctx | |
| Control 3 Temporal | 21.9 | Control (Path) 1 | 94.0 |
| Ctx | | Parietal Ctx | |
| Control 4 Temporal | 13.9 | Control (Path) 2 | 36.3 |
| Ctx | | Parietal Ctx | |
| Control (Path) 1 | 67.8 | Control (Path) 3 | 11.7 |
| Temporal Ctx | | Parietal Ctx | |
| Control (Path) 2 | 37.4 | Control (Path) 4 | 36.1 |
| Temporal Ctx | | Parietal Ctx | |

TABLE AJC
General_screening_panel_v1.4

| | Rel. Exp. (%) Ag3623, | | Rel. Exp. (%) Ag3623, |
|---|---|---|---|
| Tissue Name | Run 218211559 | Tissue Name | Run 218211559 |
| Adipose | 3.9 | Renal ca. TK-10 | 33.2 |
| Melanoma* | 22.8 | Bladder | 9.1 |
| Hs688(A).T | | | |
| Melanoma* | 27.7 | Gastric ca. (liver met.) | 29.7 |
| Hs688(B).T | | NCI-N87 | |
| Melanoma* M14 | 29.1 | Gastric ca. KATO III | 100.0 |
| Melanoma* | 27.4 | Colon ca. SW-948 | 15.0 |
| LOXIMVI | | | |
| Melanoma* SK- | 14.8 | Colon ca. SW480 | 45.4 |
| MEL-5 | | | |
| Squamous cell | 9.1 | Colon ca.* (SW480 | 23.7 |
| carcinoma SCC-4 | | met) SW620 | |
| Testis Pool | 8.4 | Colon ca. HT29 | 12.7 |
| Prostate ca.* (bone | 29.5 | Colon ca. HCT-116 | 28.7 |
| met) PC-3 | | | |
| Prostate Pool | 3.2 | Colon ca. CaCo-2 | 17.8 |
| Placenta | 16.0 | Colon cancer tissue | 13.8 |
| Uterus Pool | 1.8 | Colon ca. SW1116 | 7.4 |
| Ovarian ca. OVCAR- | 13.1 | Colon ca. Colo-205 | 11.5 |
| 3 | | | |
| Ovarian ca. SK-OV- | 39.8 | Colon ca. SW-48 | 13.5 |
| 3 | | | |
| Ovarian ca. OVCAR- | 25.0 | Colon Pool | 8.0 |
| 4 | | | |
| Ovarian ca. OVCAR- | 40.9 | Small Intestine Pool | 6.9 |
| 5 | | | |
| Ovarian ca. IGROV- | 19.9 | Stomach Pool | 3.4 |
| 1 | | | |
| Ovarian ca. OVCAR- | 14.5 | Bone Marrow Pool | 2.8 |
| 8 | | | |
| Ovary | 6.7 | Fetal Heart | 9.2 |
| Breast ca. MCF-7 | 23.3 | Heart Pool | 3.5 |
| Breast ca. MDA- | 40.3 | Lymph Node Pool | 8.7 |
| MB-231 | | | |
| Breast ca. BT 549 | 11.8 | Fetal Skeletal Muscle | 3.7 |
| Breast ca. T47D | 69.7 | Skeletal Muscle Pool | 7.8 |
| Breast ca. MDA-N | 15.4 | Spleen Pool | 3.4 |
| Breast Pool | 7.3 | Thymus Pool | 6.8 |
| Trachea | 8.8 | CNS cancer (glio/astro) | 28.9 |
| | | U87-MG | |
| Lung | 1.0 | CNS cancer (glio/astro) | 52.9 |
| | | U-118-MG | |
| Fetal Lung | 6.7 | CNS cancer (neuro; met) | 20.2 |
| | | SK-N-AS | |
| Lung ca. NCI-N417 | 7.2 | CNS cancer (astro) SF- | 12.8 |
| | | 539 | |
| Lung ca. LX-1 | 19.2 | CNS cancer (astro) | 54.3 |
| | | SNB-75 | |
| Lung ca. NCI-H146 | 12.1 | CNS cancer (glio) SNB- | 21.8 |
| | | 19 | |
| Lung ca. SHP-77 | 23.5 | CNS cancer (glio) SF- | 50.7 |
| | | 295 | |
| Lung ca. A549 | 28.7 | Brain (Amygdala) Pool | 3.7 |
| Lung ca. NCI-H526 | 13.8 | Brain (cerebellum) | 9.2 |
| Lung ca. NCI-H23 | 26.6 | Brain (fetal) | 11.3 |
| Lung ca. NCI-H460 | 7.6 | Brain (Hippocampus) | 3.6 |
| | | Pool | |
| Lung ca. HOP-62 | 14.4 | Cerebral Cortex Pool | 4.0 |
| Lung ca. MCI-H522 | 19.9 | Brain (Substantia nigra) | 5.6 |
| | | Pool | |
| Liver | 2.9 | Brain (Thalamus) Pool | 5.6 |
| Fetal Liver | 13.6 | Brain (whole) | 6.3 |
| Liver ca. HepG2 | 12.8 | Spinal Cord Pool | 5.2 |
| Kidney Pool | 18.4 | Adrenal Gland | 12.7 |
| Fetal Kidney | 5.2 | Pituitary gland Pool | 2.5 |
| Renal ca. 786-0 | 14.7 | Salivary Gland | 11.5 |
| Renal ca. A498 | 7.3 | Thyroid (female) | 13.0 |
| Renal ca. ACHN | 7.6 | Pancreatic ca. CAPAN2 | 20.9 |
| Renal ca. UO-31 | 15.9 | Pancreas Pool | 12.7 |

TABLE AJD
Panel 4.1D

| | Rel. Exp. (%) | | Rel. Exp. (%) |
|---|---|---|---|
| | Ag3623, Run | | Ag3623, Run |
| Tissue Name | 169944863 | Tissue Name | 169944863 |
| Secondary Th1 act | 73.7 | HUVEC IL-1 beta | 68.8 |
| Secondary Th2 act | 100.0 | HUVEC IFN gamma | 51.8 |
| Secondary Tr1 act | 84.7 | HUVEC TNF alpha + IFN | 49.0 |
| | | gamma | |
| Secondary Th1 rest | 16.4 | HUVEC TNF alpha + IL4 | 53.6 |
| Secondary Th2 rest | 30.4 | HUVEC IL-11 | 20.6 |
| Secondary Tr1 rest | 28.3 | Lung Microvascular EC | 79.0 |
| | | none | |
| Primary Th1 act | 88.3 | Lung Microvascular EC | 71.7 |
| | | TNFalpha + IL-1beta | |
| Primary Th2 act | 85.9 | Microvascular Dermal EC | 56.6 |
| | | none | |
| Primary Tr1 act | 97.9 | Microvascular Dermal EC | 36.6 |
| | | TNFalpha + IL-1beta | |
| Primary Th1 rest | 29.7 | Bronchial epithelium | 31.4 |
| | | TNFalpha + IL1beta | |
| Primary Th2 rest | 34.2 | Small airway epithelium | 32.3 |
| | | none | |
| Primary Tr1 rest | 39.5 | Small airway epithelium | 54.3 |
| | | TNFalpha + IL-1beta | |
| CD45RA CD4 | 76.3 | Coronery artery SMC rest | 43.2 |
| lymphocyte act | | | |
| CD45RO CD4 | 74.2 | Coronery artery SMC | 47.3 |
| lymphocyte act | | TNFalpha + IL-1beta | |
| CD8 lymphocyte act | 81.8 | Astrocytes rest | 37.1 |
| Secondary CD8 | 75.3 | Astrocytes TNFalpha + IL- | 37.4 |
| lymphocyte rest | | 1beta | |
| Secondary CD8 | 48.0 | KU-812 (Basophil) rest | 18.0 |
| lymphocyte act | | | |
| CD4 lymphocyte none | 13.0 | KU-812 (Basophil) | 47.6 |
| | | PMA/ionomycin | |
| 2ry Th1/Th2/Tr1_anti- | 36.1 | CCD1106 (Keratinocytes) | 59.9 |
| CD95 CH11 | | none | |
| LAK cells rest | 36.3 | CCD1106 (Keratinocytes) | 36.1 |
| | | TNFalpha + IL-1beta | |
| LAK cells IL-2 | 51.4 | Liver cirrhosis | 6.1 |
| LAK cells IL-2 + IL-12 | 62.4 | NCI-H292 none | 30.6 |
| LAK cells IL-2 + IFN | 67.8 | NCI-H292 IL-4 | 54.7 |
| gamma | | | |
| LAK cells IL-2 + IL-18 | 62.4 | NCI-H292 IL-9 | 74.7 |
| LAK cells | 31.6 | NCI-H292 IL-13 | 61.1 |
| PMA/ionomycin | | | |
| NK Cells IL-2 rest | 44.1 | NCI-H292 IFN gamma | 72.7 |
| Two Way MLR 3 day | 47.0 | HPAEC none | 42.3 |
| Two Way MLR 5 day | 49.3 | HPAEC TNF alpha + IL-1 | 63.3 |
| | | beta | |
| Two Way MLR 7 day | 33.4 | Lung fibroblast none | 40.6 |
| PBMC rest | 15.3 | Lung fibroblast TNF | 27.5 |
| | | alpha + IL-1 beta | |
| PBMC PWM | 79.6 | Lung fibroblast IL-4 | 67.4 |
| PBMC PHA-L | 62.4 | Lung fibroblast IL-9 | 86.5 |
| Ramos (B cell) none | 97.9 | Lung fibroblast IL-13 | 71.2 |
| Ramos (B cell) | 77.9 | Lung fibroblast IFN | 77.4 |
| ionomycin | | gamma | |
| B lymphocytes PWM | 67.8 | Dermal fibroblast | 77.9 |
| | | CCD1070 rest | |
| B lymphocytes CD40L | 39.8 | Dermal fibroblast | 87.1 |
| and IL-4 | | CCD1070 TNF alpha | |
| EOL-1 dbcAMP | 33.2 | Dermal fibroblast | 47.0 |
| | | CCD1070 IL-1 beta | |
| EOL-1 dbcAMP | 38.7 | Dermal fibroblast IFN | 40.6 |
| PMA/ionomycin | | gamma | |
| Dendritic cells none | 48.3 | Dermal fibroblast IL-4 | 75.3 |
| Dendritic cells LPS | 40.6 | Dermal Fibroblasts rest | 59.0 |
| Dendritic cells anti- | 59.5 | Neutrophils TNFa + LPS | 2.6 |
| CD40 | | | |
| Monocytes rest | 40.3 | Neutrophils rest | 9.1 |
| Monocytes LPS | 45.1 | Colon | 18.7 |
| Macrophages rest | 41.8 | Lung | 34.4 |
| Macrophages LPS | 24.3 | Thymus | 29.5 |
| HUVEC none | 41.2 | Kidney | 38.7 |
| HUVEC starved | 43.2 | | |

CNS_neurodegeneration_v1.0 Summary: Ag3623 This panel confirms the expression of the CG59859-01 gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3623 Highest expression of the CG59859-01 gene is detected in samples derived from gastric cancer KATO III cell line (CT=25). Furthermore, high expression of this gene is seen in samples derived from CNS cancer, colon cancer, gastric cancer, lung cancer, breast cancer, ovarian cancer, prostate cancer and melanoma cells. Therefore, expression of this gene can be used to distinguish these samples from other samples in the panel and also as a marker in detection of these cancers. In addition, therapeutic modulation of the activity of this gene or its protein product, through the use of small molecule drugs, protein therapeutics or antibodies, might be beneficial in the treatment of these cancers.

In addition, this gene is expressed at significant levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Among tissues with metabolic or endocrine function, this gene is expressed at low to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

Panel 4.1D Summary: Ag3623 Highest expression of the CG59859-01 gene is detected in activated secondary Th2 cells (CT=28.8). In high expression of this gene is seen in activated primary and secondary—Th1, Th2 and Tr1 cells (CTs=29) as compared to corresponding resting cells (CTs=30-31). Therefore expression of this gene can be used to distinguish between these activated versus resting cells.

Also, this gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Interestingly, expression of this gene is stimulated in PWM/PHA-L treated PBMC cells and PMA/ionomycin treated KU-812 cells (basophils) (CTs=29) as compared to the corresponding untreated/resting cells (CTs=31). Therefore, expression of this gene can be used to distinguish these treated versus untreated cells. In addition, antibody or small molecule therapies designed with the protein encoded for by this gene could block or inhibit inflammation or tissue damage due to basophil activation or PBMC activation in response to systemic lupus erythematosus, Crohn's disease, ulcerative colitis, multiple sclerosis, chronic obstructive pulmonary disease, asthma, emphysema, rheumatoid arthritis, allergies, hypersensitivity reactions, psoriasis, and viral infections.

### AK. NOV46a (CG59913-01: ATP-BINDING CASSETTE TRANSPORTER)

Expression of gene CG59913-01 was assessed using the primer-probe set Ag363 1, described in Table AKA. Results of the RTQ-PCR runs are shown in Tables AKB, AKC and AKD.

TABLE AKA
Probe Name Ag3631

| | | | | Start |
|---|---|---|---|---|
| Primers | Sequences | | Length | Position |
| Forward | 5′-cagttccaacatcaggttcagt-3′ | (SEQ ID NO:359) | 22 | 1614 |
| Probe | TET-5′-aatcacacactttcaagaatggctga-3′-TAMRA | (SEQ ID NO:360) | 26 | 1646 |
| Reverse | 5′-ttgtggacaaaatccagtgaa-3′ | (SEQ ID NO:361) | 21 | 1691 |

TABLE AKB
CNS_neurodegeneration_v1.0

| | Rel. Exp. (%) Ag3631, | | Rel. Exp. (%) Ag3631, |
|---|---|---|---|
| Tissue Name | Run 211020526 | Tissue Name | Run 211020526 |
| AD 1 Hippo | 6.8 | Control (Path) 3 | 3.3 |
| | | Temporal Ctx | |
| AD 2 Hippo | 16.7 | Control (Path) 4 | 37.6 |
| | | Temporal Ctx | |
| AD 3 Hippo | 4.2 | AD 1 Occipital Ctx | 11.9 |
| AD 4 Hippo | 3.3 | AD 2 Occipital Ctx | 0.0 |
| | | (Missing) | |
| AD 5 Hippo | 100.0 | AD 3 Occipital Ctx | 5.7 |
| AD 6 Hippo | 24.7 | AD 4 Occipital Ctx | 14.9 |
| Control 2 Hippo | 4.8 | AD 5 Occipital Ctx | 22.4 |
| Control 4 Hippo | 8.0 | AD 6 Occipital Ctx | 17.4 |
| Control (Path) 3 | 6.1 | Control 1 Occipital | 3.3 |
| Hippo | | Ctx | |
| AD 1 Temporal Ctx | 14.7 | Control 2 Occipital | 33.2 |
| | | Ctx | |
| AD 2 Temporal Ctx | 16.0 | Control 3 Occipital | 9.9 |
| | | Ctx | |
| AD 3 Temporal Ctx | 5.0 | Control 4 Occipital | 1.6 |
| | | Ctx | |
| AD 4 Temporal Ctx | 13.2 | Control (Path) 1 | 55.5 |
| | | Occipital Ctx | |
| AD 5 Inf Temporal | 61.1 | Control (Path) 2 | 10.2 |
| Ctx | | Occipital Ctx | |
| AD 5 Sup Temporal | 27.7 | Control (Path) 3 | 4.2 |
| Ctx | | Occipital Ctx | |
| AD 6 Inf Temporal | 35.6 | Control (Path) 4 | 28.9 |
| Ctx | | Occipital Ctx | |
| AD 6 Sup Temporal | 31.6 | Control 1 Parietal | 5.6 |
| Ctx | | Ctx | |
| Control 1 Temporal | 6.5 | Control 2 Parietal | 31.4 |
| Ctx | | Ctx | |
| Control 2 Temporal | 10.6 | Control 3 Parietal | 7.0 |
| Ctx | | Ctx | |
| Control 3 Temporal | 9.5 | Control (Path) 1 | 42.6 |
| Ctx | | Parietal Ctx | |
| Control 3 Temporal | 3.5 | Control (Path) 2 | 27.4 |
| Ctx | | Parietal Ctx | |
| Control (Path) 1 | 44.4 | Control (Path) 3 | 3.8 |
| Temporal Ctx | | Parietal Ctx | |
| Control (Path) 2 | 26.6 | Control (Path) 4 | 44.8 |
| Temporal Ctx | | Parietal Ctx | |

TABLE AKC
General_screening_panel_v1.4

| | Rel. Exp. (%) Ag3631, | | Rel. Exp. (%) Ag3631, |
|---|---|---|---|
| Tissue Name | Run 218233412 | Tissue Name | Run 218233412 |
| Adipose | 36.1 | Renal ca. TK-10 | 0.1 |
| Melanoma* | 3.0 | Bladder | 10.9 |
| Hs688 (A).T | | | |
| Melanoma* | 2.0 | Gastric ca. (liver met.) | 0.0 |
| Hs688 (B).T | | NCI-N87 | |
| Melanoma* M14 | 0.4 | Gastric ca. KATO III | 0.0 |
| Melanoma* | 0.0 | Colon ca. SW-948 | 0.1 |
| LOXIMVI | | | |
| Melanoma* SK- | 0.5 | Colon ca. SW480 | 0.0 |
| MEL-5 | | | |
| Squamous cell | 0.0 | Colon ca.* (SW480 | 0.0 |
| carcinoma SCC-4 | | met) SW620 | |
| Testis Pool | 14.0 | Colon ca. HT29 | 0.0 |
| Prostate ca.* (bone | 0.1 | Colon ca. HCT-116 | 0.0 |
| met) PC-3 | | | |
| Prostate Pool | 2.9 | Colon ca. CaCo-2 | 0.4 |
| Placenta | 0.2 | Colon cancer tissue | 2.1 |
| Uterus Pool | 20.4 | Colon ca. SW1116 | 0.0 |
| Ovarian ca. OVCAR- | 0.1 | Colon ca. Colo-205 | 0.0 |
| 3 | | | |
| Ovarian ca. SK-OV- | 0.7 | Colon ca. SW-48 | 0.0 |
| 3 | | | |
| Ovarian ca. OVCAR- | 0.0 | Colon Pool | 36.9 |
| 4 | | | |
| Ovarian ca. OVCAR- | 0.4 | Small Intestine Pool | 28.5 |
| 5 | | | |
| Ovarian ca. IGROV- | 0.3 | Stomach Pool | 14.5 |
| 1 | | | |
| Ovarian ca. OVCAR- | 0.0 | Bone Marrow Pool | 9.5 |
| 8 | | | |
| Ovary | 21.5 | Fetal Heart | 23.7 |
| Breast ca. MCF-7 | 0.2 | Heart Pool | 21.9 |
| Breast ca. MDA- | 0.0 | Lymph Node Pool | 27.7 |
| MB-231 | | | |
| Breast ca. BT 549 | 0.3 | Fetal Skeletal Muscle | 15.2 |
| Breast ca. T47D | 0.1 | Skeletal Muscle Pool | 21.2 |
| Breast ca. MDA-N | 3.4 | Spleen Pool | 5.8 |
| Breast Pool | 25.3 | Thymus Pool | 12.3 |
| Trachea | 16.5 | CNS cancer (glio/astro) | 0.0 |
| | | U87-MG | |
| Lung | 28.5 | CNS cancer (glio/astro) | 0.0 |
| | | U-118-MG | |
| Fetal Lung | 20.9 | CNS cancer (neuro; met) | 0.6 |
| | | SK-N-AS | |
| Lung ca. NCI-N417 | 0.0 | CNS cancer (astro) SF- | 0.0 |
| | | 539 | |
| Lung ca. LX-1 | 0.4 | CNS cancer (astro) | 0.0 |
| | | SNB-75 | |
| Lung ca. NCI-H146 | 0.2 | CNS cancer (glio) SNB- | 0.1 |
| | | 19 | |
| Lung ca. SHP-77 | 0.0 | CNS cancer (glio) SF- | 0.7 |
| | | 295 | |
| Lung ca. A549 | 0.0 | Brain (Amygdala) Pool | 4.4 |
| Lung ca. NCI-H526 | 0.0 | Brain (cerebellum) | 1.5 |
| Lung ca. NCI-H23 | 0.5 | Brain (fetal) | 3.3 |
| Lung ca. NCI-H460 | 0.4 | Brain (Hippocampus) | 4.3 |
| | | Pool | |
| Lung ca. HOP-62 | 0.3 | Cerebral Cortex Pool | 5.6 |
| Lung ca. NCI-H522 | 0.0 | Brain (Substansia nigra) | 4.4 |
| | | Pool | |
| Liver | 0.9 | Brain (Thalamus) Pool | 7.3 |
| Fetal Liver | 11.4 | Brain (whole) | 4.3 |
| Liver ca. HepG2 | 0.2 | Spinal Cord Pool | 6.7 |
| Kidney Pool | 100.0 | Adrenal Gland | 5.7 |
| Fetal Kidney | 3.6 | Pituitary gland Pool | 2.8 |
| Renal ca. 786-0 | 0.0 | Salivary Gland | 1.8 |
| Renal ca. A498 | 0.0 | Thyroid (female) | 4.2 |
| Renal ca. ACHN | 0.0 | Pancreatic ca. CAPAN2 | 0.0 |
| Renal ca. UO-31 | 1.4 | Pancreas Pool | 20.9 |

TABLE AKD
Panel 4.1D

| | Rel. Exp. (%) | | Rel. Exp. (%) |
|---|---|---|---|
| | Ag3631, Run | | Ag3631, Run |
| Tissue Name | 169960855 | Tissue Name | 169960855 |
| Secondary Th1 act | 0.0 | HUVEC IL-1 beta | 7.1 |
| Secondary Th2 act | 0.0 | HUVEC IFN gamma | 32.1 |
| Secondary Tr1 act | 0.0 | HUVEC TNF alpha + IFN | 1.2 |
| | | gamma | |
| Secondary Th1 rest | 0.8 | HUVEC TNF alpha + IL4 | 3.8 |
| Secondary Th2 rest | 2.7 | HUVEC IL-11 | 8.6 |
| Secondary Tr1 rest | 3.2 | Lung Microvascular EC | 14.8 |
| | | none | |
| Primary Th1 act | 0.0 | Lung Microvascular EC | 8.2 |
| | | TNFalpha + IL-1 beta | |
| Primary Th2 act | 0.5 | Microvascular Dermal EC | 14.7 |
| | | none | |
| Primary Tr1 act | 0.0 | Microvascular Dermal EC | 9.3 |
| | | TNFalpha + IL-1beta | |
| Primary Th1 rest | 0.9 | Bronchial epithelium | 9.6 |
| | | TNFalpha + IL1beta | |
| Primary Th2 rest | 9.3 | Small airway epithelium | 2.3 |
| | | none | |
| Primary Tr1 rest | 0.5 | Small airway epithelium | 3.0 |
| | | TNFalpha + IL-1beta | |
| CD45RA CD4 | 0.8 | Coronery artery SMC rest | 96.6 |
| lymphocyte act | | | |
| CD45RO CD4 | 1.4 | Coronery artery SMC | 81.2 |
| lymphocyte act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.6 |
| lymphocyte rest | | 1beta | |
| Secondary CD8 | 0.0 | KU-812 (Basophil) rest | 0.0 |
| lymphocyte act | | | |
| CD4 lymphocyte none | 0.4 | KU-812 (Basophil) | 0.0 |
| | | PMA/ionomycin | |
| 2ry Th1/Th2/Tr1_anti- | 3.8 | CCD1106 (Keratinocytes) | 0.0 |
| CD95 CH11 | | none | |
| LAK cells rest | 1.6 | CCD1106 (Keratinocytes) | 0.6 |
| | | TNF-alpha + IL-1beta | |
| LAK cells IL-2 | 0.0 | Liver cirrhosis | 33.0 |
| LAK cells IL-2 + IL-12 | 1.3 | NCI-H292 none | 0.0 |
| LAK cells IL-2 + IFN | 1.0 | NCI-H292 IL-4 | 0.0 |
| gamma | | | |
| LAK cells IL-2 + IL-18 | 0.3 | NCI-H292 IL-9 | 0.0 |
| LAK cells | 1.4 | NCI-H292 IL-13 | 0.0 |
| PMA/ionomycin | | | |
| NK Cells IL-2 rest | 0.6 | NCI-H292 IFN gamma | 0.0 |
| Two Way MLR 3 day | 1.9 | HPAEC none | 94.0 |
| Two Way MLR 5 day | 1.6 | HPAEC TNF alpha + IL-1 | 100.0 |
| | | beta | |
| Two Way MLR 7 day | 0.0 | Lung fibroblast none | 1.0 |
| PBMC rest | 0.0 | Lung fibroblast TNF | 0.6 |
| | | alpha + IL-1 beta | |
| PBMC PWM | 0.9 | Lung fibroblast IL-4 | 0.5 |
| PBMC PHA-L | 0.0 | Lung fibroblast IL-9 | 0.5 |
| Ramos (B cell) none | 0.0 | Lung fibroblast IL-13 | 0.0 |
| Ramos (B cell) | 0.0 | Lung fibroblast IFN | 2.2 |
| ionomycin | | gamma | |
| B lymphocytes PWM | 0.0 | Dermal fibroblast | 0.5 |
| | | CCD1070 rest | |
| B lymphocytes CD40L | 1.0 | Dermal fibroblast | 0.0 |
| and IL-4 | | CCD1070 TNF alpha | |
| EOL-1 dbcAMP | 0.9 | Dermal fibroblast | 0.4 |
| | | CCD1070 IL-1 beta | |
| EOL-1 dbcAMP | 0.5 | Dermal fibroblast IFN | 68.3 |
| PMA/ionomycin | | gamma | |
| Dendritic cells none | 0.9 | Dermal fibroblast IL-4 | 24.1 |
| Dendritic cells LPS | 2.4 | Dermal Fibroblasts rest | 27.5 |
| Dendritic cells anti- | 1.9 | Neutrophils TNFa + LPS | 0.0 |
| CD40 | | | |
| Monocytes rest | 3.0 | Neutrophils rest | 0.0 |
| Monocytes LPS | 5.9 | Colon | 17.1 |
| Macrophages rest | 1.2 | Lung | 16.7 |
| Macrophages LPS | 0.7 | Thymus | 23.7 |
| HUVEC none | 4.7 | Kidney | 13.2 |
| HUVEC starved | 4.6 | | |

CNS_neurodegeneration_v1.0 Summary: Ag3631 This panel confirms the expression of the CG59913-01 gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3631 The expression of the CG59913-01 is highest in sample derived from kidney (CT=28.23). Furhtermore, expression of this gene is very low in fetal kidney (CT=33). Thus, expression of this gene can be used in distinguishing the adult kidney from fetal kidney and also from other samples in this panel.

Among tissues with metabolic or endocrine function, this gene is expressed at low to moderate levels in pancreas, adipose, adrenal gland, thyroid, pituitary gland, skeletal muscle, heart, fetal liver and the gastrointestinal tract. Therefore, therapeutic modulation of the activity of this gene may prove useful in the treatment of endocrine/metabolically related diseases, such as obesity and diabetes.

In addition, this gene is expressed at significant levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

CG59913-01 gene codes for a variant of ATP-binding casette A9 protein, an ATP-binding cassette (ABC) transporter belonging to ABCA sub-family. The ABC superfamily comprises of myriad transmembrane proteins involved in the transport of vitamins, peptides, steroid hormones, ions, sugars, and amino acids (ref. 1). Known genetic diseases resulting from dysfunctional ABC transporters are cystic fibrosis, Zellweger syndrome, adrenoleukodystrophy, multidrug resistance, Stargardt macular dystrophy, Tangier disease (TD) and familial HDL deficiency (FHA) (ref. 2, 3). Recently, it has been shown that functional loss of ABCA1, a transporter belonging to ABCA subfamily, in mice causes severe placental malformation, aberrant lipid distribution, and kidney glomerulonephritis, as well as, high-density lipoprotein cholesterol deficiency (ref 3). CG59913-01 gene is expressed in large number of the normal tissue used in this panel. In analogy to ABCA1, this gene may also play a wider role in lipid metabolism, renal inflammation, and cardiovascular disease and CNS disorders.

References.

1. Higgins C F. (1992) ABC transporters: from microorganisms to man. Annu Rev Cell Biol 8:67-113

PMID: 1282354

2. Decottignies A, Goffeau A. (1997) Complete inventory of the yeast ABC proteins. Nat Genet 15(2):137-45.

The complete sequence of the yeast genome predicts the existence of 29 proteins belonging to the ubiquitous ATP-binding cassette (ABC) superfamily. Using binary comparison, phylogenetic classification and detection of conserved amino acid residues, the yeast ABC proteins have been classified in a total of six clusters, including ten subclusters of distinct predicted topology and presumed distinct function. Study of the yeast ABC proteins provides insight into the physiological function and biochemical mechanisms of their human homologues, such as those involved in cystic fibrosis, adrenoleukodystrophy, Zellweger syndrome, multidrug resistance and the antiviral activity of interferons.

PMID: 9020838

3. Christiansen-Weber T A, Voland J R, Wu Y, Ngo K, Roland B L, Nguyen S, Peterson P A, Fung-Leung W P. (2000) Functional loss of ABCA1 in mice causes severe placental malformation, aberrant lipid distribution, and kidney glomerulonephritis as well as high-density lipoprotein cholesterol deficiency. Am J Pathol September 2000;157(3):1017-29

Tangier disease (TD) and familial HDL deficiency (FHA) have recently been linked to mutations in the human ATP-binding cassette transporter 1 (hABCA1), a member of the ABC superfamily. Both diseases are characterized by the lowering or lack of high-density lipoprotein cholesterol (HDL-C) and low serum cholesterol. The murine ABCA1−/− phenotype corroborates the human TD linkage to ABCA1. Similar to TD in humans, HDL-C is virtually absent in ABCA1−/− mice accompanied by a reduction in serum cholesterol and lipid deposition in various tissues. In addition, the placenta of ABCA1−/− mice is malformed, resulting in severe embryo growth retardation, fetal loss, and neonatal death. The basis for these defects appears to be altered steroidogenesis, a direct result of the lack of HDL-C. By 6 months of age, ABCA1−/− animals develop membranoproliferative glomerulonephritis due to deposition of immunocomplexes followed by cardiomegaly with ventricular dilation and hypertrophy, ultimately succumbing to congestive heart failure. This murine model of TD will be very useful in the study of lipid metabolism, renal inflammation, and cardiovascular disease and may reveal previously unsuspected relationships between them.

PMID: 10980140

Panel 4.1D Summary: Ag3631 The expression of the CG59913-01 gene is high in sample derived from HPAEC and coronary artery SMC cell lines(CTs=30). Thus, expression of this gene can be used to distinguish these samples from other samples in this panel.

Interestingly, expression of this gene is stimulated on treatment of dermal fibroblast and HUVEC cells with IFN gama (CTs=31-32). Therefore, therapeutics designed with the protein encoded for by this transcript could be important treating diseases such as asthma, arthritis, psoriasis, IBD, and systemic lupus erythematosus.

Significant expression of this gene is also detected in a liver cirrhosis sample (CT=32). Furthermore, expression of this gene is low/undetectable in normal liver (CT=35)in Panel 1.4, suggesting that its expression is unique to liver cirrhosis. Therefore, antibodies or small molecule therapeutics could reduce or inhibit fibrosis that occurs in liver cirrhosis.

This gene is expressed at moderate levels in the normal colon, lung, thymus and kidney tissues (CTs=32-33). This ubiquitous pattern of expression in normal tissues suggests that this gene product may be involved in homeostatic processes for these tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

### AL. NOV47a (CG59909-01: ATP-BINDING CASSETTE TRANSPORTER)

Expression of gene CG59909-01 was assessed using the primer-probe set Ag3630, described in Table ALA. Results of the RTQ-PCR runs are shown in Tables ALB, ALC and ALD.

TABLE ALA
Probe Name Ag3630

| | | | | Start |
|---|---|---|---|---|
| Primers | Sequences | | Length | Position |
| Forward | 5′-ttccaacaccaaaggactaatg-3′ | (SEQ ID NO:362) | 22 | 1339 |
| Probe | TET-5′-aaatcgatgctgagcatccctctgat-3′-TAMRA | (SEQ ID NO:363) | 26 | 1379 |
| Reverse | 5′-ttcaggagctactggttcaaaa-3′ | (SEQ ID NO:364) | 22 | 1410 |

TABLE ALB
CNS_neurodegeneration_v1.0

| | Rel. Exp.(%) | Rel. Exp.(%) | | Rel. Exp.(%) | Rel. Exp.(%) |
|---|---|---|---|---|---|
| | Ag3630, Run | Ag3630, Run | Tissue | Ag3630, Run | Ag3630, Run |
| Tissue Name | 211020447 | 224079049 | Name | 211020447 | 224079049 |
| AD 1 Hippo | 10.7 | 10.7 | Control | 4.4 | 3.2 |
| | | | (Path) 3 | | |
| | | | Temporal | | |
| | | | Ctx | | |
| AD 2 Hippo | 26.1 | 25.5 | Control | 21.6 | 22.4 |
| | | | (Path) 4 | | |
| | | | Temporal | | |
| | | | Ctx | | |
| AD 3 Hippo | 6.5 | 8.4 | AD 1 | 19.9 | 26.8 |
| | | | Occipital | | |
| | | | Ctx | | |
| AD 4 Hippo | 9.3 | 8.1 | AD 2 | 0.0 | 0.0 |
| | | | Occipital | | |
| | | | Ctx | | |
| | | | (Missing) | | |
| AD 5 hippo | 77.9 | 79.0 | AD 3 | 8.8 | 12.6 |
| | | | Occipital | | |
| | | | Ctx | | |
| AD 6 Hippo | 64.2 | 51.4 | AD 4 | 20.4 | 24.3 |
| | | | Occipital | | |
| | | | Ctx | | |
| Control 2 Hippo | 10.8 | 11.4 | AD 5 | 29.7 | 35.1 |
| | | | Occipital | | |
| | | | Ctx | | |
| Control 4 Hippo | 6.8 | 5.5 | AD 6 | 18.7 | 22.1 |
| | | | Occipital | | |
| | | | Ctx | | |
| Control (Path) 3 | 9.1 | 9.9 | Control 1 | 1.9 | 3.3 |
| Hippo | | | Occipital | | |
| | | | Ctx | | |
| AD 1 Temporal | 26.1 | 29.7 | Control 2 | 30.8 | 35.6 |
| Ctx | | | Occipital | | |
| | | | Ctx | | |
| AD 2 Temporal | 21.0 | 31.2 | Control 3 | 19.5 | 23.5 |
| Ctx | | | Occipital | | |
| | | | Ctx | | |
| AD 3 Temporal | 8.0 | 8.7 | Control 4 | 2.4 | 3.3 |
| Ctx | | | Occipital | | |
| | | | Ctx | | |
| AD 4 Temporal | 20.6 | 25.5 | Control | 53.2 | 81.2 |
| Ctx | | | (Path) 1 | | |
| | | | Occipital | | |
| | | | Ctx | | |
| AD 5 Inf | 100.0 | 100.0 | Control | 10.9 | 14.0 |
| Temporal Ctx | | | (Path) 2 | | |
| | | | Occipital | | |
| | | | Ctx | | |
| AD 5 | 32.5 | 50.7 | Control | 5.4 | 5.4 |
| SupTemporal | | | (Path) 3 | | |
| Ctx | | | Occipital | | |
| | | | Ctx | | |
| AD 6 Inf | 56.3 | 69.3 | Control | 17.0 | 23.7 |
| Temporal Ctx | | | (Path) 4 | | |
| | | | Occipital | | |
| | | | Ctx | | |
| AD 6 Sup | 52.1 | 62.4 | Control 1 | 4.4 | 4.9 |
| Temporal Ctx | | | Parietal Ctx | | |
| Control 1 | 4.0 | 6.0 | Control 2 | 57.4 | 54.3 |
| Temporal Ctx | | | Parietal Ctx | | |
| Control 2 | 10.0 | 20.9 | Control 3 | 18.4 | 20.9 |
| Temporal Ctx | | | Parietal Ctx | | |
| Control 3 | 11.3 | 12.5 | Control | 29.3 | 48.0 |
| Temporal Ctx | | | (Path) 1 | | |
| | | | Parietal Ctx | | |
| Control 4 | 7.1 | 7.2 | Control | 19.8 | 24.0 |
| Temporal Ctx | | | (Path) 2 | | |
| | | | Parietal Ctx | | |
| Control (Path) 1 | 29.1 | 31.6 | Control | 4.8 | 5.5 |
| Temporal Ctx | | | (Path) 3 | | |
| | | | Parietal Ctx | | |
| Control (Path) 2 | 20.9 | 25.5 | Control | 38.2 | 45.4 |
| Temporal Ctx | | | (Path) 4 | | |
| | | | Parietal Ctx | | |

TABLE ALC
General_screening_panel_v1.4

| | Rel. Exp. (%) Ag3630, | | Rel. Exp. (%) Ag3630, |
|---|---|---|---|
| Tissue Name | Run 218212091 | Tissue Name | Run 218212091 |
| Adipose | 0.9 | Renal ca. TK-10 | 0.0 |
| Melanoma* | 3.6 | Bladder | 52.9 |
| Hs688 (A).T | | | |
| Melanoma* | 1.7 | Gastric ca. (liver met.) | 0.7 |
| Hs688 (B).T | | NCI-N87 | |
| Melanoma* M14 | 0.4 | Gastric ca. KATO III | 0.0 |
| Melanoma* | 0.1 | Colon ca. SW-948 | 0.0 |
| LOXIMVI | | | |
| Melanoma* SK- | 0.5 | Colon ca. SW480 | 0.0 |
| MEL-5 | | | |
| Squamous cell | 0.0 | Colon ca.* (SW480 | 0.1 |
| carcinoma SCC-4 | | met) SW620 | |
| Testis Pool | 11.8 | Colon ca. HT29 | 0.0 |
| Prostate ca.* (bone | 0.2 | Colon ca. HCT-116 | 0.2 |
| met) PC-3 | | | |
| Prostate Pool | 6.4 | Colon ca. CaCo-2 | 0.4 |
| Placenta | 0.2 | Colon cancer tissue | 5.7 |
| Uterus Pool | 20.7 | Colon ca. SW1116 | 0.0 |
| Ovarian ca. OVCAR- | 0.0 | Colon ca. Colo-205 | 0.8 |
| 3 | | | |
| Ovarian ca. SK-OV- | 0.9 | Colon ca. SW-48 | 0.0 |
| 3 | | | |
| Ovarian ca. OVCAR- | 0.0 | Colon Pool | 38.7 |
| 4 | | | |
| Ovarian ca. OVCAR- | 0.8 | Small Intestine Pool | 41.8 |
| 5 | | | |
| Ovarian ca. IGROV- | 0.2 | Stomach Pool | 31.4 |
| 1 | | | |
| Ovarian ca. OVCAR- | 0.1 | Bone Marrow Pool | 13.0 |
| 8 | | | |
| Ovary | 47.6 | Fetal Heart | 20.7 |
| Breast ca. MCF-7 | 0.7 | Heart Pool | 18.4 |
| Breast ca. MDA- | 0.2 | Lymph Node Pool | 24.1 |
| MB-231 | | | |
| Breast ca. BT 549 | 0.9 | Fetal Skeletal Muscle | 27.0 |
| Breast ca. T47D | 0.5 | Skeletal Muscle Pool | 29.5 |
| Breast ca. MDA-N | 0.0 | Spleen Pool | 21.5 |
| Breast Pool | 39.0 | Thymus Pool | 20.7 |
| Trachea | 24.1 | CNS cancer (glio/astro) | 0.1 |
| | | U87-MG | |
| Lung | 29.9 | CNS cancer (glio/astro) | 0.0 |
| | | U-118-MG | |
| Fetal Lung | 100.0 | CNS cancer (neuro; met) | 0.0 |
| | | SK-N-AS | |
| Lung ca. NCI-N417 | 0.0 | CNS cancer (astro) SF- | 0.1 |
| | | 539 | |
| Lung ca. LX-1 | 0.0 | CNS cancer (astro) | 0.1 |
| | | SNB-75 | |
| Lung ca. NCI-H146 | 0.0 | CNS cancer (glio) SNB- | 0.2 |
| | | 19 | |
| Lung ca. SHP-77 | 0.0 | CNS cancer (glio) SF- | 5.3 |
| | | 295 | |
| Lung ca. A549 | 0.0 | Brain (Amygdala) Pool | 7.6 |
| Lung ca. NCI-H526 | 0.0 | Brain (cerebellum) | 5.0 |
| Lung ca. NCI-H23 | 3.7 | Brain (fetal) | 2.5 |
| Lung ca. NCI-H460 | 3.0 | Brain (Hippocampus) | 7.8 |
| | | Pool | |
| Lung ca. HOP-62 | 2.5 | Cerebral Cortex Pool | 9.7 |
| Lung ca. NCI-H522 | 0.0 | Brain (Substantia nigra) | 6.2 |
| | | Pool | |
| Liver | 5.8 | Brain (Thalamus) Pool | 15.3 |
| Fetal Liver | 80.1 | Brain (whole) | 11.7 |
| Liver ca. HepG2 | 0.0 | Spinal Cord Pool | 21.2 |
| Kidney Pool | 79.0 | Adrenal Gland | 16.3 |
| Fetal Kidney | 5.2 | Pituitary gland Pool | 3.7 |
| Renal ca. 786-0 | 0.0 | Salivary Gland | 4.6 |
| Renal ca. A498 | 0.1 | Thyroid (female) | 3.2 |
| Renal ca. ACHN | 0.0 | Pancreatic ca. CAPAN2 | 0.0 |
| Renal ca. UO-31 | 0.2 | Pancreas Pool | 25.5 |

TABLE ALD
Panel 4.1D

| | Rel. Exp.(%) | | Rel. Exp.(%) |
|---|---|---|---|
| | Ag3630, Run | | Ag3630, Run |
| Tissue Name | 169960853 | Tissue Name | 169960853 |
| Secondary Th1 act | 0.4 | HUVEC IL-1 beta | 20.3 |
| Secondary Th2 act | 0.3 | HUVEC IFN gamma | 57.4 |
| Secondary Tr1 act | 0.0 | HUVEC TNF alpha + IFN | 8.5 |
| | | gamma | |
| Secondary Th1 rest | 0.3 | HUVEC TNF alpha + IL4 | 8.0 |
| Secondary Th2 rest | 0.7 | HUVEC IL-11 | 28.5 |
| Secondary Tr1 rest | 0.2 | Lung Microvascular EC | 10.4 |
| | | none | |
| Primary Th1 act | 0.2 | Lung Microvascular EC | 6.6 |
| | | TNF alpha + IL-1 beta | |
| Primary Th2 act | 0.0 | Microvascular Dermal EC | 24.3 |
| | | none | |
| Primary Tr1 act | 0.2 | Microsvasular Dermal EC | 16.7 |
| | | TNF alpha + IL-1 beta | |
| Primary Th1 rest | 0.5 | Bronchial epithelium | 3.0 |
| | | TNF alpha + IL1 beta | |
| Primary Th2 rest | 0.3 | Small airway epithelium | 3.5 |
| | | none | |
| Primary Tr1 rest | 0.7 | Small airway epithelium | 2.2 |
| | | TNF alpha + IL-1 beta | |
| CD45RA CD4 | 1.0 | Coronery artery SMC rest | 68.3 |
| lymphocyte act | | | |
| CD45RO CD4 | 0.1 | Coronery artery SMC | 44.1 |
| lymphocyte act | | TNF alpha + IL-1 beta | |
| CD8 lymphocyte act | 0.0 | Astrocytes rest | 0.0 |
| Secondary CD8 | 0.0 | Astrocytes TNF alpha + IL- | 0.1 |
| lymphocyte rest | | 1 beta | |
| Secondary CD8 | 0.0 | KU-812 (Basophil) rest | 0.0 |
| lymphocyte act | | | |
| CD4 lymphocyte none | 1.7 | KU-812 (Basophil) | 0.0 |
| | | PMA/ionomycin | |
| 2ry Th1/Th2/Tr1_anti- | 0.8 | CCD1106 (Keratinocytes) | 0.5 |
| CD95 CH11 | | none | |
| LAK cells rest | 1.9 | CCD1106 (Keratinocytes) | 0.6 |
| | | TNF alpha + IL-1 beta | |
| LAK cells IL-2 | 0.3 | Liver cirrhosis | 37.1 |
| LAK cells IL-2 + IL-12 | 0.4 | NCI-H292 none | 0.2 |
| LAK cells IL-2 + IFN | 1.7 | NCI-H292 IL-4 | 0.0 |
| gamma | | | |
| LAK cells IL-2 + IL-18 | 1.6 | NCI-H292 IL-9 | 0.0 |
| LAK cells | 0.6 | NCI-H292 IL-13 | 0.0 |
| PMA/ionomycin | | | |
| NK Cells IL-2 rest | 0.4 | NCI-H292 IFN gamma | 0.0 |
| Two Way MLR 3 day | 1.1 | HPAEC none | 73.2 |
| Two Way MLR 5 day | 0.4 | HPAEC TNF alpha + IL-1 | 100.0 |
| | | beta | |
| Two Way MLR 7 day | 0.0 | Lung fibroblast none | 1.8 |
| PBMC rest | 0.1 | Lung fibroblast TNF | 6.0 |
| | | alpha + IL-1 beta | |
| PBMC PWM | 0.2 | Lung fibroblast IL-4 | 1.3 |
| PBMC PHA-L | 0.2 | Lung fibroblast IL-9 | 1.6 |
| Ramos (B cell) none | 0.0 | Lung fibroblast IL-13 | 1.3 |
| Ramos (B cell) | 0.0 | Lung fibroblast IFN | 2.0 |
| ionomycin | | gamma | |
| B lymphocytes PWM | 0.0 | Dermal fibroblast | 0.3 |
| | | CCD1070 rest | |
| B lymphocytes CD40L | 0.9 | Dermal fibroblast | 0.3 |
| and IL-4 | | CCD1070 TNF alpha | |
| EOL-1 dbcAMP | 0.0 | Dermal fibroblast | 0.5 |
| | | CCD1070 IL-1 beta | |
| EOL-1 dbcAMP | 0.0 | Dermal fibroblast IFN | 53.6 |
| PMA/ionomycin | | gamma | |
| Dendritic cells none | 2.5 | Dermal fibroblast IL-4 | 37.6 |
| Dendritic cells LPS | 2.8 | Dermal Fibroblasts rest | 25.2 |
| Dendritic cells anti- | 4.9 | Neutrophils TNFa + LPS | 0.0 |
| CD40 | | | |
| Monocytes rest | 0.4 | Neutrophils rest | 0.3 |
| Monocytes LPS | 5.9 | Colon | 11.3 |
| Macrophages rest | 0.9 | Lung | 27.4 |
| Macrophages LPS | 8.7 | Thymus | 24.7 |
| HUVEC none | 5.8 | Kidney | 19.8 |
| HUVEC starved | 9.8 | | |

CNS_neurodegeneration_v1.0 Summary: Ag3630,Two experiments with the same probe and primer set produce results that are in excellent agreement. This panel confirms the expression of this gene at low to moderate levels in the brains of an independent group of individuals. Expression of this gene is found to be up-regulated in the temporal cortex of Alzheimer's disease patients. Therefore, blockade of this protein product may be of use in reversing the dementia/memory loss associated with Alzheimer's disease and neuronal death.

General_screening_panel_v1.4 Summary: Ag3630 The CG59909-01 gene is expressed primarily in normal tissue samples, with highest expression in the fetal lung (CT=26.3).

This gene has low-to-moderate expression in adipose, liver, heart, skeletal muscle, adrenal, pituitary and pancreas. By homology, this gene product is a lipid or cholesterol transporter and can be expected to play a critical role in metabolic processes. Therapeutic modulation of ag3630 may be a treatment for endocrine or metabolic disease, including Types 1 and 2 diabetes and obesity.

Interestingly, this gene is expressed at much higher levels in fetal (CT=26.3) when compared to adult liver (CT=30.5). This observation suggests that expression of this gene can be used to distinguish fetal from adult liver.

In addition, this gene is expressed at high levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4.1D Summary: Ag3630 Highest expression of the CG59909-01 gene is detected in TNF alpha+IL-1beta treated HPAEC (CT=28.5). In addition, this gene is expressed in endothelium, smooth muscle cells and fibroblasts. It is also expressed in normal kidney, lung, colon, and thymus. The transcript was particularly induced by gamma interferon in HUVEC cells. HUVEC cells have been used in many in vitro models to examine leukocyte extravsation. Gamma interferon treatment induces the expression many proteins in HUVEC cells that induce leukocyte rolling and binding, necessary steps in the movement of leukocytes from the blood into the periphery. The putative transporter encoded for by this transcript could be important in endothelium mediated leukocyte recruitment and thus be an important target for the treatment of inflammation associated with asthma, emphysema, psoriasis, and arthritis.

Reference.

1. Lidington E A, Moyes D L, McCormack A M, Rose M L. (1999) A comparison of primary endothelial cells and endothelial cell lines for studies of immune interactions. Transpl Immunol December 1999;7(4):239-46

### AM. NOV48a (CG59945-01: STEROID HORMONE RECEPTOR)

Expression of gene CG59945-01 was assessed using the primer-probe sets Ag3632 and ag3666, described in Tables AMA and AMB. Results of the RTQ-PCR runs are shown in Tables AMC, and AMD.

TABLE AMA
Probe Name Ag3632

| | | | | Start |
|---|---|---|---|---|
| Primers | Sequences | | Length | Position |
| Forward | 5′-atggtggttgagcctgaga-3′ | (SEQ ID NO:365) | 19 | 904 |
| Probe | TET-5′-agctctatgccttgcccgaccct-3′-TAMRA | (SEQ ID NO:366) | 23 | 923 |
| Reverse | 5′-tcggtcaaagaggtcacaga-3′ | (SEQ ID NO:367) | 20 | 983 |

TABLE AMB
Probe Name ag3666

| | | | | Start |
|---|---|---|---|---|
| Primers | Sequences | | Length | Position |
| Forward | 5′-gttcccttgatggaaaatgaag-3′ | (SEQ ID NO:368) | 21 | 14 |
| Probe | TET-5′-cctcatctactgactctcccatccaa-3′-TAMRA | (SEQ ID NO:369) | 26 | 35 |
| Reverse | 5′-gtgggaacttttgtcctcctt-3′ | (SEQ ID NO:370) | 21 | 76 |

TABLE AMC
CNS_neurodegeneration_v1.0

| | Rel. Exp.(%) Ag3632, | | Rel. Exp.(%) Ag3632, |
|---|---|---|---|
| Tissue Name | Run 211020527 | Tissue Name | Run 211020527 |
| AD 1 Hippo | 26.8 | Control (Path) 3 | 0.0 |
| | | Temporal Ctx | |
| AD 2 Hippo | 40.9 | Control (Path) 4 | 4.8 |
| | | Temporal Ctx | |
| AD 3 Hippo | 28.1 | AD 1 Occipital Ctx | 1.7 |
| AD 4 Hippo | 0.0 | AD 2 Occipital Ctx | 0.0 |
| | | (Missing) | |
| AD 5 hippo | 36.3 | AD 3 Occipital Ctx | 0.0 |
| AD 6 Hippo | 100.0 | AD 4 Occipital Ctx | 1.7 |
| Control 2 Hippo | 44.8 | AD 5 Occipital Ctx | 1.1 |
| Control 4 Hippo | 22.7 | AD 6 Occipital Ctx | 21.5 |
| Control (Path) 3 | 50.3 | Control 1 Occipital | 0.0 |
| Hippo | | Ctx | |
| AD 1 Temporal Ctx | 0.0 | Control 2 Occipital | 7.4 |
| | | Ctx | |
| AD 2 Temporal Ctx | 1.1 | Control 3 Occipital | 1.4 |
| | | Ctx | |
| AD 3 Temporal Ctx | 0.0 | Control 4 Occipital | 1.5 |
| | | Ctx | |
| AD 4 Temporal Ctx | 36.3 | Control (Path) 1 | 49.0 |
| | | Occipital Ctx | |
| AD 5 Inf Temporal | 13.2 | Control (Path) 2 | 0.0 |
| Ctx | | Occipital Ctx | |
| AD 5 SupTemporal | 41.8 | Control (Path) 3 | 1.2 |
| Ctx | | Occipital Ctx | |
| AD 6 Inf Temporal | 42.6 | Control (Path) 4 | 17.8 |
| Ctx | | Occipital Ctx | |
| AD 6 Sup Temporal | 77.4 | Control 1 Parietal | 0.0 |
| Ctx | | Ctx | |
| Control 1 Temporal | 0.0 | Control 2 Parietal | 16.4 |
| Ctx | | Ctx | |
| Control 2 Temporal | 7.5 | Control 3 Parietal | 0.0 |
| Ctx | | Ctx | |
| Control 3 Temporal | 1.1 | Control (Path) 1 | 14.6 |
| Ctx | | Parietal Ctx | |
| Control 4 Temporal | 1.9 | Control (Path) 2 | 7.9 |
| Ctx | | Parietal Ctx | |
| Control (Path) 1 | 6.3 | Control (Path) 3 | 0.0 |
| Temporal Ctx | | Parietal Ctx | |
| Control (Path) 2 | 7.6 | Control (Path) 4 | 6.7 |
| Temporal Ctx | | Parietal Ctx | |

TABLE AMD
General_screening_panel_v1.4

| | Rel. Exp.(%) Ag3632, | | Rel. Exp.(%) Ag3632, |
|---|---|---|---|
| Tissue Name | Run 218233723 | Tissue Name | Run 218233723 |
| Adipose | 0.0 | Renal ca. TK-10 | 0.0 |
| Melanoma* | 0.0 | Bladder | 0.0 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* | 0.0 | Colon ca. SW480 | 0.0 |
| SK-MEL-5 | | | |
| Squamous cell | 0.0 | Colon ca.* (SW480 | 0.0 |
| carcinoma SCC-4 | | met) SW620 | |
| Testis Pool | 100.0 | Colon ca. HT29 | 3.3 |
| Prostate ca.* (bone | 0.0 | Colon ca. HCT-116 | 0.0 |
| met) PC-3 | | | |
| Prostate Pool | 0.0 | Colon ca. CaCo-2 | 0.0 |
| Placenta | 1.5 | Colon cancer tissue | 0.0 |
| Uterus Pool | 4.6 | Colon ca. SW1116 | 0.0 |
| Ovarian ca. OVCAR-3 | 0.0 | Colon ca. Colo-205 | 0.0 |
| Ovarian ca. SK-OV-3 | 2.4 | Colon ca. SW-48 | 0.0 |
| Ovarian ca. OVCAR-4 | 0.0 | Colon Pool | 0.0 |
| Ovarian ca. OVCAR-5 | 0.0 | Small Intestine Pool | 0.0 |
| Ovarian ca. IGROV-1 | 0.0 | Stomach Pool | 0.0 |
| Ovarian ca. OVCAR-8 | 0.0 | Bone Marrow Pool | 0.0 |
| Ovary | 0.0 | Fetal Heart | 0.0 |
| Breast ca. MCF-7 | 0.0 | Heart Pool | 0.0 |
| Breast ca. MDA- | 0.0 | Lymph Node Pool | 0.0 |
| MB-231 | | | |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 0.0 | CNS cancer (glio/astro) | 0.0 |
| | | U87-MG | |
| Lung | 0.0 | CNS cancer (glio/astro) | 0.0 |
| | | U-118-MG | |
| Fetal Lung | 0.0 | CNS cancer (neuro; met) | 0.0 |
| | | SK-N-AS | |
| Lung ca. NCI-N417 | 0.0 | CNS cancer (astro) SF- | 0.0 |
| | | 539 | |
| Lung ca. LX-1 | 0.0 | CNS cancer (astro) | 0.0 |
| | | SNB-75 | |
| Lung ca. NCI-H146 | 0.0 | CNS cancer (glio) SNB- | 0.0 |
| | | 19 | |
| Lung ca. SHP-77 | 0.0 | CNS cancer (glio) SF- | 0.0 |
| | | 295 | |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) | 11.9 |
| | | Pool | |
| Lung ca. HOP-62 | 0.0 | Cerebral Cortex Pool | 0.0 |
| Lung ca. NCI-H522 | 0.0 | Brain (Substantia nigra) | 0.0 |
| | | Pool | |
| Liver | 0.0 | Brain (Thalamus) Pool | 2.9 |
| Fetal Liver | 0.0 | Brain (whole) | 0.0 |
| Liver ca. HepG2 | 0.0 | Spinal Cord Pool | 0.0 |
| Kidney Pool | 1.6 | Adrenal Gland | 0.0 |
| Fetal Kidney | 1.9 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 3.3 |
| Renal ca. UO-31 | 0.0 | Pancreas Pool | 0.0 |

CNS_neurodegeneration_v1.0 Summary: Ag3632 This panel confirms the expression of The CG59945-01 gene at significant levels in the brain in an independent group of individuals. This gene is found to be upregulated in the temporal cortex of Alzheimer's disease patients. Blockade of this receptor may be of use in the treatment of this disease and decrease neuronal death.

The CG59945-01 gene, a steroid hormone receptor homolog. Steroid hormones play a role in brain development and modulating emotion, among other functions. Based on the expression of this gene in the brain and its homology to a steroid hormone receptor, this gene product may play a role in normal neurologic development and function.

Ag3666 Results from one experiment with this gene are not included. The amp plot indicates that there were experimental difficulties with this run.

References:

1. Kawata M, Matsuda K, Nishi M, Ogawa H, Ochiai I. Intracellular dynamics of steroid hormone receptor. Neurosci Res July 2001;40(3):197-203

General_screening_panel_v1.4 Summary: Ag3632 Expression of the CG59945-01 gene is restricted to the testis (CT=33.9). Thus, expression of this gene could be used to differentiate between this sample and other samples on this panel and as a marker of testicular tissue. Furthermore, therapeutic modulation of the expression or function of this gene may be useful in the treatment of male infertility or hypogonadism.

General_screening_panel_v1.5 Summary: Ag3666 Expression of the CG59945-01 gene is low/undetectable in all samples on this panel (CTs>35).

Panel 4.1D Summary: Ag3632/Ag3666 Expression of the CG59945-01 gene is low/undetectable in all samples on this panel (CTs>35).

Panel CNS_{—}1 Summary: Ag3666 Expression of the CG59945-01 gene is low/undetectable in all samples on this panel (CTs>35).

### Example D

Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8) 1249-1265, 2000).

Variants are reported individually but any combination of all or a select subset of variants are also included as contemplated NOVX embodiments of the invention.

### RESULTS

### NOV2a SNP Data

One polymorphic variant of NOV2a has been identified and is shown in Table 3A.

TABLE 3A

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377213 | 69 | T | C | 15 | Thr | Thr |

### NOV4a SNP Data

Four polymorphic variants of NOV5a have been identified and are shown in Table 3B.

TABLE 3B

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377217 | 137 | A | T | 40 | Gln | His |
| 13377216 | 231 | A | C | 72 | Ile | Leu |
| 13377215 | 401 | G | A | 128 | Val | Val |
| 13377214 | 417 | C | T | 134 | Gln | End |

### NOV5a SNP Data

Four polymorphic variants of NOV5a have been identified and are shown in Table 3C.

TABLE 3C

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13375650 | 1871 | A | G | 616 | Lys | Arg |
| 13375649 | 1895 | A | G | 624 | Asn | Ser |
| 13375648 | 1900 | A | G | 626 | Thr | Ala |
| 13375647 | 1931 | T | C | 636 | Val | Ala |

### NOV7a SNP Data

Two polymorphic variants of NOV7a have been identified and arc shown in Table 3D.

TABLE 3D

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377218 | 90 | C | T | 0 | | |
| 13377219 | 185 | T | C | 29 | Leu | Pro |

### NOV10s SNP Data

Two polymorphic variants of NOV10a have been identified and are shown in Table 3E.

TABLE 3E

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377221 | 668 | T | C | 6 | Leu | Leu |
| 13377220 | 803 | A | T | 51 | Leu | Leu |

### NOV11a SNP Data

Seven polymorphic variants of NOV11a have been identified and are shown in Table 3F.

TABLE 3F

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13374741 | 856 | A | G | 271 | Pro | Pro |
| 13377222 | 994 | C | T | 317 | Cys | Cys |
| 13377223 | 1234 | A | G | 397 | Pro | Pro |
| 13377224 | 1417 | C | T | 458 | Cys | Cys |
| 13374755 | 1736 | G | A | 565 | Gly | Ser |
| 13377226 | 2012 | G | T | 657 | Glu | End |
| 13377227 | 2073 | T | C | 677 | Leu | Pro |

### NOV15a SNP Data

One polymorphic variant of NOV15a has been identified and is shown in Table 3G.

TABLE 3G

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377231 | 202 | C | T | 54 | Asp | Asp |

### NOV19a SNP Data

Two polymorphic variants of NOV19a has been identified and are shown in Table 3H.

TABLE 3H

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377233 | 348 | C | T | 86 | His | His |
| 13377232 | 516 | T | C | 142 | His | His |

### NOV21a SNP Data

One polymorphic variant of NOV21 a has been identified and is shown in Table 3I.

TABLE 3I

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377234 | 438 | T | C | 125 | Phe | Phe |

### NOV23a SNP Data

Eight polymorphic variants of NOV23a have been identified and are shown in Table 3J.

TABLE 3J

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13374631 | 498 | A | G | 142 | Gln | Arg |
| 13374630 | 643 | G | T | 190 | Gln | His |
| 13374633 | 672 | A | G | 200 | Asp | Gly |
| 13377237 | 715 | C | T | 214 | Ser | Ser |
| 13377238 | 903 | C | T | 277 | Thr | Ile |
| 13374632 | 983 | A | G | 304 | Lys | Glu |
| 13377240 | 1419 | T | C | 449 | Val | Ala |
| 13374629 | 1547 | T | C | 492 | Ser | Pro |

### NOV24a SNP Data

Six polymorphic variants of NOV24a have been identified and are shown in Table 3K.

TABLE 3K

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377245 | 596 | C | T | 0 | | |
| 13377246 | 779 | A | C | 42 | Ser | Arg |
| 13377244 | 1327 | G | A | 224 | Pro | Pro |
| 13377243 | 1586 | G | A | 311 | Ala | Thr |
| 13377242 | 1764 | C | T | 0 | | |
| 13377241 | 1825 | A | G | 0 | | |

### NOV25a SNP Data

Eight polymorphic variants of NOV25a have been identified and are shown in Table 3L.

TABLE 3L

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13375267 | 2963 | A | G | 925 | Leu | Leu |
| 13375266 | 3213 | C | T | 1009 | Pro | Ser |
| 13375265 | 3313 | T | C | 1042 | Val | Ala |
| 13375264 | 3981 | G | A | 1265 | Val | Met |
| 13375263 | 4023 | A | G | 1279 | Thr | Ala |
| 13375262 | 4051 | G | T | 1288 | Gly | Val |
| 13375261 | 4105 | A | G | 1306 | Asn | Ser |
| 13375260 | 4117 | T | C | 1310 | Ile | Thr |

### NOV27a SNP Data

Three polymorphic variants of NOV27a have been identified and are shown in Table 3M.

TABLE 3M

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377249 | 233 | T | C | 0 | | |
| 13377248 | 1714 | C | T | 419 | Thr | Thr |
| 13377247 | 2008 | A | G | 517 | Gln | Gln |

### NOV29a SNP Data

Three polymorphic variants of NOV29a have been identified and are shown in Table 3N.

TABLE 3N

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13375558 | 132 | G | A | 44 | Leu | Leu |
| 13375557 | 149 | A | C | 50 | Lys | Thr |
| 13375556 | 187 | T | C | 63 | Ser | Pro |

### NOV30a SNP Data

Six polymorphic variants of NOV30a have been identified and are shown in Table 3O.

TABLE XX

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13374752 | 1871 | A | G | 599 | Met | Val |
| 13374753 | 1970 | A | G | 632 | Met | Val |
| 13374754 | 2001 | T | C | 642 | Phe | Ser |
| 13377254 | 2249 | C | T | 725 | Leu | Leu |
| 13377253 | 3186 | A | C | 1037 | Gln | Pro |
| 13377252 | 3318 | C | T | 0 | | |

### NOV31 a SNP Data

Fifteen polymorphic variants of NOV31a have been identified and are shown in Table 3P.

TABLE 3P

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377256 | 1335 | C | T | 434 | Asn | Asn |
| 13377257 | 1415 | A | G | 461 | Asp | Gly |
| 13377258 | 1476 | A | G | 481 | Ala | Ala |
| 13377259 | 1526 | T | C | 498 | Val | Ala |
| 13377260 | 1613 | G | A | 527 | Cys | Tyr |
| 13377261 | 1630 | A | G | 533 | Thr | Ala |
| 13377262 | 1812 | A | T | 593 | Ala | Ala |
| 13377263 | 1929 | G | A | 632 | Ala | Ala |
| 13377264 | 1942 | C | T | 637 | Arg | Trp |
| 13377265 | 2343 | T | C | 770 | Gly | Gly |
| 13374745 | 2418 | C | T | 795 | Ser | Ser |
| 13374744 | 2535 | C | T | 834 | Leu | Leu |
| 13374743 | 2549 | A | G | 839 | Asp | Gly |
| 13377266 | 2642 | T | C | 870 | Leu | Pro |
| 13377267 | 2649 | C | T | 872 | Asp | Asp |

### NOV33a SNP Data

Two polymorphic variants of NOV33a have been identified and are shown in Table 3Q.

TABLE 3Q

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377270 | 79 | T | C | 17 | Leu | Pro |
| 13377269 | 400 | T | C | 124 | Val | Ala |

### NOV36a SNP Data

Three polymorphic variants of NOV36a have been identified and are reported in Table 3R.

TABLE 3R

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377273 | 91 | C | G | 24 | Ala | Ala |
| 13377272 | 230 | T | C | 71 | Ser | Pro |
| 13377271 | 4250 | A | G | 1411 | Thr | Ala |

### NOV37a SNP Data

Two polymorphic variants of NOV37a have been identified and are reported in Table 3S.

TABLE 3S

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377274 | 266 | A | G | 73 | Arg | Arg |
| 13377276 | 702 | A | G | 219 | Ile | Val |

### NOV39a SNP Data

Two polymorphic variants of NOV39a have been identified and are shown in Table 3T.

TABLE 3T

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377277 | 6369 | T | C | 2123 | Ser | Ser |
| 13377278 | 8094 | G | A | 2698 | Pro | Pro |

### NOV40a SNP Data

One polymorphic variant of NOV40a has been identified and is shown in Table 3U.

TABLE 3U

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377279 | 355 | G | A | 115 | Ala | Thr |

### NOV42a SNP Data

One polymorphic variant of NOV42a has been identified and is shown in Table 3V.

TABLE 3V

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377280 | 698 | A | G | 221 | Leu | Leu |

### NOV43a SNP Data

One polymorphic variant of NOV43a has been identified and is shown in Table 3W.

TABLE 3W

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377281 | 1371 | T | C | 433 | His | His |

### NOV44a SNP Data

Four polymorphic variants of NOV44a have been identified and are shown in Table 3X.

TABLE 3X

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377282 | 604 | A | G | 45 | Asn | Ser |
| 13377283 | 684 | A | G | 72 | Thr | Ala |
| 13377284 | 1123 | G | A | 218 | Arg | His |
| 13377285 | 1489 | T | C | 340 | Val | Ala |

### NOV45a SNP Data

One polymorphic variant of NOV45a has been identified and is shown in Table 3Y.

TABLE 3Y

| | Nucleotides | | | Amino Acids | | |
|---|---|---|---|---|---|---|
| | Base | | | Base | | |
| Variant | Position of | Wild- | | Position of | Wild- | |
| No. | SNP | type | Variant | SNP | type | Variant |
| 13377286 | 624 | T | C | 191 | Ser | Ser |

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. The claims presented are representative of the inventions disclosed herein. Other, unclaimed inventions are also contemplated. Applicants reserve the right to pursue such inventions in later claims.

We claim:

1. An isolated polypeptide comprising an amino acid sequence selected from the group consisting of:
a) a mature form of the amino acid sequence selected from the group consisting of SEQ ID NO:2n, wherein n is an integer between 1-101;
b) a variant of a mature form of the amino acid sequence selected from the group consisting of SEQ ID NO:2n, wherein n is an integer between 1-101, wherein any amino acid in the mature form is changed to a different amino acid, provided that no more than 15% of the amino acid residues in the sequence of the mature form are so changed;
c) the amino acid sequence selected from the group consisting of SEQ ID NO:2n, wherein n is an integer between 1-101;
d) a variant of the amino acid sequence selected from the group consisting of SEQ ID NO:2n, wherein n is an integer between 1-101, wherein any amino acid specified in the chosen sequence is changed to a different amino acid, provided that no more than 15% of the amino acid residues in the sequence are so changed; and
e) a fragment of any of a) through d).

2. The polypeptide of claim 1 that is a naturally occurring allelic variant of the sequence selected from the group consisting of SEQ ID NO:2n, wherein n is an integer between 1-101.

3. The polypeptide of claim 2, wherein said allelic variant comprises an amino acid sequence that is the translation of a nucleic acid sequence differing by a single nucleotide from a nucleic acid sequence selected from the group consisting of SEQ ID NO:2n−1, wherein n is an integer between 1-101.

4. The polypeptide of claim 1 that is a variant polypeptide described therein, wherein any amino acid specified in the chosen sequence is changed to provide a conservative substitution.

5. A pharmaceutical composition comprising the polypeptide of claim 1 and a pharmaceutically acceptable carrier.

6. A kit comprising in one or more containers, the pharmaceutical composition of claim 5.

7. The use of a therapeutic in the manufacture of a medicament for treating a syndrome associated with a human disease, the disease selected from a pathology associated with the polypeptide of claim 1, wherein said therapeutic is the polypeptide of claim 1.

8. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide, thereby determining the presence or amount of polypeptide in said sample.

9. A method for determining the presence of or predisposition to a disease associated with altered levels of the polypeptide of claim 1 in a first mammalian subject, the method comprising:
a) measuring the level of expression of the polypeptide in a sample from the first mammalian subject; and
b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

10. A method for modulating the activity of the polypeptide of claim 1, the method comprising introducing a cell sample expressing the polypeptide of said claim with an antibody that binds to said polypeptide in an amount sufficient to modulate the activity of the polypeptide.

11. The method of claim 10, wherein said subject is a human.

12. An isolated nucleic acid molecule comprising a nucleic acid sequence encoding a polypeptide comprising an amino acid sequence selected from the group consisting of:
a) a mature form of the amino acid sequence of SEQ ID NO:2n, wherein n is an integer between 1-101;
b) a variant of a mature form of the amino acid sequence selected from the group consisting of SEQ ID NO:2n, wherein n is an integer between 1-101, wherein any amino acid in the mature form of the chosen sequence is changed to a different amino acid, provided that no more than 15% of the amino acid residues in the sequence of the mature form are so changed;
c) the amino acid sequence selected from the group consisting of SEQ ID NO:2n, wherein n is an integer between 1-101;
d) a variant of the amino acid sequence selected from the group consisting of SEQ ID NO:2n, wherein n is an integer between 1-101, in which any amino acid specified in the chosen sequence is changed to a different amino acid, provided that no more than 15% of the amino acid residues in the sequence are so changed;
e) a nucleic acid fragment encoding at least a portion of a polypeptide comprising the amino acid sequence selected from the group consisting of SEQ ID NO:2n, wherein n is an integer between 1-101, or any variant of said polypeptide wherein any amino acid of the chosen sequence is changed to a different amino acid, provided that no more than 10% of the amino acid residues in the sequence are so changed; and
f) the complement of any of said nucleic acid molecules.

13. The nucleic acid molecule of claim 12, wherein the nucleic acid molecule comprises the nucleotide sequence of a naturally occurring allelic nucleic acid variant.

14. The nucleic acid molecule of claim 12 that encodes a variant polypeptide, wherein the variant polypeptide has the polypeptide sequence of a naturally occurring polypeptide variant.

15. The nucleic acid molecule of claim 12, wherein the nucleic acid molecule differs by a single nucleotide from a nucleic acid sequence selected from the group consisting of SEQ ID NO:2n−1, wherein n is an integer between 1-101.

16. The nucleic acid molecule of claim 12, wherein said nucleic acid molecule comprises a nucleotide sequence selected from the group consisting of
a) the nucleotide sequence selected from the group consisting of SEQ ID NO:2n−1, wherein n is an integer between 1-101;
b) a nucleotide sequence wherein one or more nucleotides in the nucleotide sequence selected from the group consisting of SEQ ID NO:2n−1, wherein n is an integer between 1-101, is changed from that selected from the group consisting of the chosen sequence to a different nucleotide provided that no more than 15% of the nucleotides are so changed;
c) a nucleic acid fragment of the sequence selected from the group consisting of SEQ ID NO:2n−1, wherein n is an integer between 1-101; and
d) a nucleic acid fragment wherein one or more nucleotides in the nucleotide sequence selected from the group consisting of SEQ ID NO:2n−1, wherein n is an integer between 1-101, is changed from that selected from the group consisting of the chosen sequence to a different nucleotide provided that no more than 15% of the nucleotides are so changed.

17. The nucleic acid molecule of claim 12, wherein said nucleic acid molecule hybridizes under stringent conditions to the nucleotide sequence selected from the group consisting of SEQ ID NO:2n−1, wherein n is an integer between 1-101, or a complement of said nucleotide sequence.

18. The nucleic acid molecule of claim 12, wherein the nucleic acid molecule comprises a nucleotide sequence in which any nucleotide specified in the coding sequence of the chosen nucleotide sequence is changed from that selected from the group consisting of the chosen sequence to a different nucleotide provided that no more than 15% of the nucleotides in the chosen coding sequence are so changed, an isolated second polynucleotide that is a complement of the first polynucleotide, or a fragment of any of them.

19. A vector comprising the nucleic acid molecule of claim 12.

20. The vector of claim 19, further comprising a promoter operably linked to said nucleic acid molecule.

21. A cell comprising the vector of claim 20.

22. A method for determining the presence or amount of the nucleic acid molecule of claim 12 in a sample, the method comprising:
(a) providing said sample;
(b) introducing said sample to a probe that binds to said nucleic acid molecule; and
(c) determining the presence or amount of said probe bound to said nucleic acid molecule,
thereby determining the presence or amount of the nucleic acid molecule in said sample.

23. The method of claim 22 wherein presence or amount of the nucleic acid molecule is used as a marker for cell or tissue type.

24. The method of claim 23 wherein the cell or tissue type is cancerous.

25. A method for determining the presence of or predisposition to a disease associated with altered levels of the nucleic acid molecule of claim 12 in a first mammalian subject, the method comprising:
a) measuring the amount of the nucleic acid in a sample from the first mammalian subject; and
b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

26. An antibody that binds immunospecifically to the polypeptide of claim 1.

27. The antibody of claim 26, wherein said antibody is a monoclonal antibody.

28. The antibody of claim 26, wherein the antibody is a humanized antibody.

29. The antibody of claim 26, wherein the antibody is a fully human antibody

30. The antibody of claim 26, wherein the dissociation constant for the binding of the polypeptide to the antibody is less than 1×10⁻⁹ M.

31. The antibody of claim 26, wherein the antibody neutralizes an activity of the polypeptide.

32. A pharmaceutical composition comprising the antibody of claim 26 and a pharmaceutically acceptable carrier.

33. A kit comprising in one or more containers, the pharmaceutical composition of claim 29.

34. The use of a therapeutic in the manufacture of a medicament for treating a syndrome associated with a human disease, the disease selected from a pathology associated with the polypeptide of claim 1, wherein said therapeutic is a NOVX antibody.

35. A method of treating or preventing a NOVX-associated disorder, said method comprising administering to a subject in which such treatment or prevention is desired the antibody of claim 26 in an amount sufficient to treat or prevent said NOVx-associated disorder in said subject.

36. A method of treating a pathological state in a mammal, the method comprising administering to the mammal the antibody of claim 26 in an amount sufficient to alleviate the pathological state.

37. A method of treating or preventing a pathology associated within the polypeptide of claim 1, said method comprising administering to a subject in which such treatment or prevention is desired a NOVX antibody in an amount sufficient to treat or prevent said pathology in said subject.

38. The method of claim 37, wherein the subject is a human.
